SEQUENCE LISTING (735 sequences)

SEQ ID NO: 1 - protein, 7 aa, synthetic construct
  REGION 1..7: note = Synthetic construct
VVVVVVD

SEQ ID NO: 2 - protein, 11 aa, synthetic construct
  REGION 1..11: note = Synthetic construct
QQKFQFQFEQQ

SEQ ID NO: 3 - protein, 6 aa, synthetic construct
  REGION 1..6: note = Synthetic construct
KAAAAK

SEQ ID NO: 4 - protein, 8 aa, synthetic construct
  REGION 1..8: note = Synthetic construct
KAAAAAAK

SEQ ID NO: 5 - protein, 8 aa, synthetic construct
  REGION 1..8: note = Synthetic construct
RAAAAAAR

SEQ ID NO: 6 - protein, 24 aa, synthetic construct
  REGION 1..24: note = Synthetic construct
KLEALYVLGFFGFFTLGIMLSYIR

SEQ ID NO: 7 - protein, 21 aa, synthetic construct
  REGION 1..21: note = Synthetic construct
QATNRNTDGSTDYGILQINSR

SEQ ID NO: 8 - protein, 16 aa, synthetic construct
  REGION 1..16: note = Synthetic construct
RERERKRKRERERKRK

SEQ ID NO: 9 - protein, 11 aa, synthetic construct
  REGION 1..11: note = Synthetic construct
QQRFQWQFEQQ

SEQ ID NO: 10 - protein, 11 aa, synthetic construct
  REGION 1..11: note = Synthetic construct
QQRFEWEFEQQ

SEQ ID NO: 11 - protein, 16 aa, synthetic construct
  REGION 1..16: note = Synthetic construct
RADARADARADARADA

SEQ ID NO: 12 - protein, 12 aa, synthetic construct
  REGION 1..12: note = Synthetic construct
KLDLKLDLKLDL

SEQ ID NO: 13 - protein, 18 aa, Carnobacterium maltaromaticum
MKTVKELSVKEMQLTTGG

SEQ ID NO: 14 - protein, 18 aa, Carnobacterium maltaromaticum
MNSVKELNVKEMKQLHGG

SEQ ID NO: 15 - protein, 18 aa, Carnobacterium maltaromaticum
MKSVKELNKKEMQQINGG

SEQ ID NO: 16 - protein, 18 aa, Enterococcus faecium
MKHLKILSIKETQLIYGG

SEQ ID NO: 17 - protein, 15 aa, Enterococcus faecium
MEKLTVKEMSQVVGG

SEQ ID NO: 18 - protein, 15 aa, Escherichia coli
MRTLTLNELDSVSGG

SEQ ID NO: 19 - protein, 15 aa, Escherichia coli
MREITLNEMNNVSGA

SEQ ID NO: 20 - protein, 15 aa, Escherichia coli
MRELDREELNCVGGA

SEQ ID NO: 21 - protein, 18 aa, Escherichia coli
MANIRELTLDEITLVSGG

SEQ ID NO: 22 - protein, 18 aa, Escherichia coli
MSNIRELSFDEIALVSGG

SEQ ID NO: 23 - protein, 15 aa, Escherichia coli
MREITESQLRYISGA

SEQ ID NO: 24 - protein, 15 aa, Escherichia coli
MREISDNMLDSVKGG

SEQ ID NO: 25 - protein, 15 aa, Escherichia coli
MRKLSENEIKQISGG

SEQ ID NO: 26 - protein, 15 aa, Klebsiella pneumoniae
MREISQKDLNLAFGA

SEQ ID NO: 27 - protein, 15 aa, Klebsiella pneumoniae
MRALTENDFFAVSGA

SEQ ID NO: 28 - protein, 18 aa, Lactobacillus curvatus
MNNVKELSMTELQTITGG

SEQ ID NO: 29 - protein, 18 aa, Lactobacillus sakei
MNNVKELSMTELQTITGG

SEQ ID NO: 30 - protein, 18 aa, Lactobacillus sakei
MEKFIELSLKEVTAITGG

SEQ ID NO: 31 - protein, 24 aa, Leuconostoc carnosum
MNNMKSADNYQQLDNNALEQVVGG

SEQ ID NO: 32 - protein, 24 aa, Leuconostoc gelidum
MMNMKPTESYEQLDNSALEQVVGG

SEQ ID NO: 33 - protein, 24 aa, Leuconostoc mesenteroides
MTNMKSVEAYQQLDNQNLKKVVGG

SEQ ID NO: 34 - protein, 15 aa, Mixta gaviniae
MRMLINNDIKLVTGA

SEQ ID NO: 35 - protein, 15 aa, Pantoea agglomerans
MRELNSEEIRNVAGA

SEQ ID NO: 36 - protein, 16 aa, Pantoea allii
MLRELTDSEINIVSGG

SEQ ID NO: 37 - protein, 18 aa, Pediococcus acidilactici
MKKIEKLTEKEMANIIGG

SEQ ID NO: 38 - protein, 21 aa, Streptococcus uberis
MNTIEKFENIKLFSLKKIIGG

SEQ ID NO: 39 - protein, 19 aa, unidentified
  source 1..19: note = Synechococcus
MSQFINLINDQNAQELLGG

SEQ ID NO: 40 - protein, 19 aa, unidentified
  source 1..19: note = Synechococcus
MSKLINTIDDNHAQSLVGG

SEQ ID NO: 41 - protein, 19 aa, unidentified
  source 1..19: note = Synechococcus
MNQWLESINDDTAQQLLGG

SEQ ID NO: 42 - protein, 22 aa, unidentified
  source 1..22: note = Synechococcus
MQVEFDLITDLSDGQAEMVTGA

SEQ ID NO: 43 - protein, 15 aa, Vibrio cholerae
MKELSIEQISLVTGG

SEQ ID NO: 44 - protein, 698 aa, Klebsiella pneumoniae
MSNGNVRRMINQLDMRWRRRVPVIHQTETSECGLACLAMICGHFGKNIDLISLRRKFNLSARGANLAGINSIAEQLGMVTRALSLELDELGALKTPCILHWDFSHFVVLVSVKRNRYVLHDPARGRRNVGLEEMTRYFTGVALEVWPGSEFSEETTQNRIQLRSLINSIYGIKSTLAKIFCLSVVIEAINLVMPVGTQLVMDHAIPAGDRGLLTLISAGLMFFILLRAATGMLRAWSSLVMGTLINVQWQSGLFNHLLRLPLAYFERRKLGDIQSRFGSLDTLRATFTTSVVGAIMDSIMVVGVFVMMLLYGGYLTWIVLGFTTVYVLIRLVTYGYYRQISEETLVRGARASSYFMETLYGIATVKIQGMAERRGTHWLNLKIDAINSGIKLTKMDLFFGGINTFVAACDQVAILWLGTSLVIDNQMTIGMFVAFGSFRGQFSDRVASLTNFLLQLRMMSLHNERIADIALHEKEEKKPELDIVADMTPVSLETTDLSYRYDSQSAPVFSGLYLSVTPGESVAITGTSGAGKTTLMKVLCGLFEPDSGKVLVNGTDIRQLGINNYHRMIACVMQDDRLFSGSIRENICGFSEEMDEAWMIECARASYIHDVIMKMPMGYETLIGELGEGLSGGQKQRIFIARALYRRPGILFMDEATSSLDTESERFVNVAIRNMNITRVIIAHRETTLRTVDRTISI

SEQ ID NO: 45 - protein, 702 aa, Chania multitudinisentens
MNKLSFKCLIKQLDIRLFQRIPLVHQTEASECGLACLAMICGHYGKNIDLIALRQQFNLAARGTTLASLTGIADQLGLATRPLSLDLNELSALKKPCILHWEFNHFVVLVSVGRHRVVLHDPARGRRTVSLSELSSSFTGVALEVWPGGAFTADTIRDRFSFGTLIGSVHGLKGTLGKIFCLSLVIETINLVMPVGTQLVMDHAIPAGDRGLLTLICAGLMLFILLRTAVSMVRAWSSLVMSTLINVQWQSGLFNHLLRLPLGYFERRKLGDIQSRFGSLDTLRQTFTTSIVGAIMDGIMIMGVLVMMVLYGGWLTWVVVAFTTLYVLLRLITYVYYRQLSEESLIRKARAGSYFMETLYGIATVKMQGMTERRSAHWLNLEIDAINTGIRVTKMDMLFGGINTFVAACDQIVILWLGTRLVIDTQMTIGMFVAFAAFRGQFSDRISSLTEFLLQLRLMSLHNERIADIALHPRENRQPAIVYEPQLRPATLTTQALSYSYDSQSPPIFNKLDIVVAPGESVAIVGPSGAGKTTLMKVLCGLFLPDHGKVEINGVDIQQLGINNYHKMIACVMQDDKLFSGSIRENISGFSDDVDEDWMQECARASYLHDVIMRMPMRYETLIGELGEGLSGGQKQRLFIARAIYKKPTILFLDEATSALDKESETTVNQSIKALRITRIIIAHRESTIASADRVITLNPQL

SEQ ID NO: 46 - protein, 699 aa, unidentified
  source 1..699: note = Kosakonia
MSNKSLFTALLGKLDLSWFRRVPMVHQTESSECGLASLAMICGHYGKNIDLISLRQQFNLSARGTTLIGLNEIAGQLGLATRALSLDLHELKLLKTPCVLHWDFNHFVVLVAVKGNKYVINDPARGRRTVGQAEMSQYFTGVALEAMPGSEFTQEKVKNRVRLRSLIGSVHGIKATLAKIFCLSIVIEAINLVLPVGTQLVMDHAIPAGDRGLLSLICIGLFFFILLRASVSMLRAWTSLVMSTLINVQWQSGLFRHLIKLPLGYFERRKMGDIQSRFGSLDTLRETFTSSVVGAIMDGIMVVGVLIMLVLYGGWLTWVVLGFTAVYVLIRLLTYNYYRQLSEERLVRGARASSYFMETLYGIATVKMQDMSERRGTHWLNLQVDTINTGIKLTRLDLFFNGLNTFIAACDQVAILWLGTSLVIDNQMTIGMFVAFGAFRGQFSDRIGSLVDFLLQLRMMSLHNERISDIALHEREPRKPDLPATTEMRPLGLETTGLSYRYDSQSAPIFNDLALRIEPGESVALVGASGAGKTTLMKVLCGLFEPDAGKVLVDGMDIKQLGINNYHKMIGCVLQDDKLFAGSLRENICGFAEHADEEWMVECAKASHIHDVILAMPMGYETLIGELGEGLSGGQKQRVFIARALYRKPGLLFMDEATSALDHESEACVNNAIKQLNITRVIIAHRATTIATADRVINL

SEQ ID NO: 47 - protein, 699 aa, Kosakonia oryzae
MSNKSLFSALLGKLDLSWFRRVPMVHQTESSECGLASLAMICGHYGKNIDLISLRQQFNLSARGTTLIGLNEIAGQLGLATRALSLDMHELKLLKTPCVLHWDFNHFVVLVAVKGNKYVINDPARGRRTVGQAEMSQYFTGVALEAMPGSEFTQEKVKNRVRLRSLVGSVHGIKATLAKIFCLSIVIEAINLVLPVGTQLVMDHAIPAGDRGLLSLICIGLFFFIMLRASVSMLRAWTSLVMSTLINVQWQSGLFRHLIKLPLGYFERRKMGDIQSRFGSLDTLRETFTSSVVGAIMDGIMVVGVLIMLVLYGGWLTWVVLGFTAVYVLIRLLTYNYYRQLSEERLVRGARASSYFMETLYGIATVKMQDMSERRGTHWLNLQVDTINTGIKLTRLDFFFNGLNTFIAACDQVAILWLGTSLVIDNQMTIGMFVAFGAFRGQFSDRVGSLVEFLLQLRMMSLHNERISDIALHEREPRKPDLPATTEMRPLGLETTGLSYRYDSQSAPIFNDLALRIEPGESVALVGASGAGKTTLMKVLCGLFEPDAGKVLVDGMDIKQLGINNYHKMIGCVLQDDKLFAGSLRENICGFAEHADEEWMVKCAKASHIHDVILAMPMGYETLIGELGEGLSGGQKQRVFIARALYRKPGLLFMDEATSALDHESEACVNNAIKQLNITRVIIAHRATTIATADRVINL

SEQ ID NO: 48 - protein, 710 aa, Limnobaculum parvum
MDKATFTHLLESLNFGMRKKVPQILQTEAAECGLACLAMISRYYGMNVDLFNLRRRFGISSHGATLGVVINTASEINLKSRALTLDLNEINQLKTPCILHWDLNHFVVLVEVKRNKFVLHDPAFGRRVVSEQEMSQHFTGVALEVWPGNEFVPYNETSRLSLWSMMSHIEGLKGFLVKVFALSIVIETVNLLIPVGTQLVMDHVILAEDHDLLGLICLGLLFFILFRTFVGMLRSWTSLVMEALVGVQWKAGLFDHLMKLPLSYFEKRKLGDIQSRFISLDTIRTTLTSNLVSSIIDSLMAIGVFIMMLLYGGWLVWVVLGFTVLYMILRLSTYQHYRQASEEQIIKGAKANSHFMETLYGISTLKALGLTATRGQYWLNLNIDTTNANIRLTKLDMVFGGMNTLLGMVDQIVILWLGASMVIDGHMTLGMFVAFNAYRGQFSERSANLINLALELKMLSLHSERLTDIVFSEPENTLPNNMVFPEDQPLGFEVRHVEFRYDNLSAPIFSNLNLKIEPGENVAIIGPSGIGKTTLMKVMAGLLLPSQGEVLANGIDINKIGVNNYRRYIACVLQDDKLFAGSIADNIASFEAQKDSAWVVECAKHCNIHEEIMRMPMGYETLITELGGSLSGGQKQRLLIARALYRRPSLLFLDEATSHLDTANEAHINAAISALKVTRIFIAHRLSTIESADRVINLDPVEEFVAVTPD

SEQ ID NO: 49 - protein, 699 aa, unidentified
  source 1..699: note = Rahnella
MNLDNVRELADRLHFSWRHRVPQIIQTEAAECGLASLAMVFGYHGKHVDLQSLRQRYGISSRGATLRTLMDIAAANEMKSRALKLDIDELNALKTPCILHWDLNHFVVLVGVKQGKVIIHDPAFGRRSLGMREVSEHFTGVALELWPDNGFTSEKPRVRLDLRKMMGNVSGLIPALTKIFCLSLMIESVNLLLPVGMQLVMDHVIPAKDPDLLTLICLGLLFFIIFRTGVSMLRAWTSLVMSTLIDVQWKARLFDHLMKLPLDYFEKRKLGDIQSRFTSLDTLRTTLTTNVVNSIIDGIMSVGLIVMMVLYGGWLIWVILGFTAIYVIFRLTTYNAYRQVSEEQIVKGARAGSHFMETLYGISTLKALGLSKARANFWLNLNIDNANATVRKTRFEMMFTGGNTLIATLDQVALLWLGATQVIDGHMTLGMFVAFNTYRGQFSERAASLLNMVLQLRMLSLHRDRIADIALTGTEKSLPERELLRAGEAASLEVRDLVFQYDSLSAPLINGLNLSVAAGESVAITGPSGQGKTTLMKIMTGLLTPTEGRILINGMDITTAGLNNYRSCIACVLQDDTLFAGSIAENIASFDEQKDEARIIDCARRCNIHDDIERMPMGYETLISELGGSLSGGQKQRLLIARALYRRPAILFLDEATSHLDLDNEMRINQAISELDITRIFIAHRPSTIASADREIKLG

SEQ ID NO: 50 - protein, 699 aa, unidentified
  source 1..699: note = Rahnella
MNLDNVRELADRLHFSWRHRVPQIIQTEAAECGLASLAMVFGYHGKHVDLQSLRQRYGISSRGATLRTLMDIAAANEMKSRALKLDIDELNALKTPCILHWDLNHFVVLVGVKQGKVIIHDPAFGRRSLGMREVSEHFTGVALELWPDNGFTSEKPRVRLDLRKMMGNVSGLIPALTKIFCLSLMIESVNLLLPVGMQLVMDHVIPAKDPDLLTLICLGLLFFIIFRTGVSMLRAWTSLVMSTLIDVQWKARLFDHLMKLPLDYFEKRKLGDIQSRFTSLDTLRTTLTTNVVNSIIDGIMSVGLIVMMVLYGGWLIWVILGFTAIYVIFRLTTYNAYRQVSEEQIVKGARAGSHFMETLYGISTLKALGLSKARANFWLNLNIDNANATVRKTRFEMMFTGGNTLIATLDQVALLWLGATQVIDGHMTLGMFVAFNTYRGQFSERAASLLNMVLQLRMLSLHRDRIADIALTDTEKSLPERELLRAGEAASLEVRDLVFQYDSLSAPLINGLNLSVAAGESVAITGPSGQGKTTLMKIMTGLLTPTEGRILINGMDITTAGLNNYRSCIACVLQDDTLFAGSIAENIASFDEQKDEARIIDCARRCNIHDDIERMPMGYETLISELGGSLSGGQKQRLLIARALYRRPAILFLDEATSHLDLDNEMRINQAISELDITRIFIAHRPSTIASADREIKLG

SEQ ID NO: 51 - protein, 703 aa, unidentified
  source 1..703: note = Raoultella
MNKDVLDSIFKRINFSMKNKVPVIIQSESTECGNACLSMICGFYGKDIDLFNFRNRYGSTSQGATLNVLAAIAQKAGLKTRALSLDIAEIKELRLPCILHWSLNHFVVLVAIKGKRFIIHDPALGRRVVHLQELSENFSGIALEAWPDSDFRQEKQRSRLKLLDLMHNMVGLKSALIKIFMLSVVIETVNLLLPMGTQIVTDHVITAHDENLLLVICVGLMFFTIFKTWVSMIRAWVSLKLNTLTDVQWKTSFFDHLMSLPLAFFEKRQLGDIQSRFASLDIIRATFTNSIVTGMIDSIMTIGLLIMLSLYGGWLVWVVLGFTVCYAIMRALTYKFYRTVSEELIVKRARSGSHFMESLYGIATIKSLNLKNRRSQHWLNTNIDVSNAGLKQTRFDMLFGGINTFINSADQVVILWLGAQMVMDNTMTIGMFMAFNAYRGQFTQRAASLIDLTMQLKMLSLHNERISEIVYSEPEVDSPLRNVFEENVGVSLEVRDLAYQYDLLSKPVFSNVNISVAAGESVALVGVSGIGKTTLLKVMSGLLTPERGEIFIGGFDINKIGINNFRSNIACVLQEDRLFSGSITDNISGFDDEVDEALVIECAMQCNIHEEILRMPMGYETIIGELGAGISGGQKQRLLIARALYQKPRILFMDEATSHLDINNEKIINAAIESLNITRIIIAHRPSTIACADRIIDLAKITP

SEQ ID NO: 52 - protein, 704 aa, Salmonella enterica
MQQGRSMSNGNVRRMINQLDMRWRRRVPVIHQTETSECGLACLAMICGHFGKNIDLISLRRKFNLSSRGANLAGINSIAEQLGMVTRALSLELDELGALKTPCILHWDFSHFVVLVSVKRNRYVLHDPARGRRNVGLEEMTRYFTGVALEVWPGSEFSAETTQNRIHLRSLINSIYGIKSTLAKIFCLSVVIEAINLIMPVGTQLVMDHAIPAGDRGLLTLISAGLMFFILLRAATGMLRAWSSLVMGTLINVQWQSGLFNHLLRLPLAYFERRKLGDIQSRFGSLDTLRATFTTSVVGAIMDSIMVVGVFVMMLLYGGYLTWIVLGFTTVYVLVRLVTYSYYRQISEETLVRGARASSYFMETLYGIATVKIQGMAERRGTHWLNLKIDAINSGIKLTKMDLFFGGINTFVAACDQVAILWLGTSLVIDNQMTIGMFVAFGSFRGQFSDRVASLTNFLLQLRMVSLHNERIADIALHEKEEKKPELDIVADMSPVSLETTDLSYRYDSQSAPVFSGLYLSVTPGESVAITGTSGAGKTTLMKVLCGLFEPDTGKVLVNGTDIRQLGINNYHRMIACVMQDDRLFSGSIRENICGFSEEMDETWMIECARASYIHDVIIKMPMGYETLIGELGEGLSGGQKQRIFIARALYRRPCILFMDEATSSLDTDSERFVNVAIKNMNITRIIIAHRETTLRTVDRVILI

SEQ ID NO: 53 - protein, 704 aa, Salmonella enterica
MQQGRSMSNGNIRRMINQLDMRWRGRVPVIHQTETSECGLACLAMICGHFGKNIDLISLRRKFNLSSRGANLAGINSIAEQLGMVTRALSLELDELGALKTPCILHWDFSHFVVLVSVKRNRYVLHDPARGRRNVGLEEMTRYFTGVALEVWPGSEFSAETTQNRIHLRSLINSIYGIKSTLAKIFCLSVVIEAINLIMPVGTQLVMDHAIPAGDRGLLTLISAGLMFFILLRAATGMLRAWSSLVMGTLINVQWQSGLFNHLLRLPLAYFERRKLGDIQSRFGSLDTLRATFTTCVVGAIMDSIMVVGVFVMMLLYGGYLTWIVLGFTTAYVLIRLVTYSYYRQISEETLVRGARASSYFMETLYGIATVKIQGMAERRGTHWLNLKIDAINSGIKLTKMDLFFGGINTFVAACDQVAILWLGTSLVIDNQMTIGMFVAFGSFRGQFSDRVASLTNNLLQLRMVSLHNERIADIALHEKEEKKPELDIVADMSPVSLETTDLSYRYDNQSAPVFSGLYLSVTPGESVAITGTSGAGKTTLMKVLCGLFEPDTGKVLVNGTDIRQLGINNYHRMIACVMQDDRLFSGSIRENICGFSEEMDETWMIECARASYIHDVIIKMPMGYETLIGELGEGLSGGQKQRIFIARALYRRPCILFMDEATSSLDTDSERFVNVAIKNMNITRIIIAHRETTLRTVDRVILI

SEQ ID NO: 54 - protein, 710 aa, unidentified
  source 1..710: note = Serratia
MKDHNLFERINEKLIFSLRKRVPSILQSESSECGLACLAMISSYYGFNVDMLSLRQRFGISTQGATLGTISQIASQIQLKTRALSLDIDEVNQLKTPCVLHWNMNHFVVLVKVRRASFVIHDPAFGRRVIGLQEMSNHFTGIALELWPDRAFQKETLKTRLRLLDLMKNIEGLPGTLLKIFALSIVIESVNLLLPVGTQLVTDHVIQAHDYSLLTVICLGLIFFTLFRAVVSIARAWISIVLGTLTDIQWKTTLFEHLLKLPLDFFEKRHLGDIQSRFSSLDAIRTTFTNNIVSGIIDGIMTVGLFVMMMVYGGWLVWVVSGFTLVYILIRMMTYRTYRQFSEEQIVKAAKANSHFMETLYGVSTVKALGIKDTRSSYWLNLNVDAANTNIKITRFNMMFGGINTFITTLDQVAILWLGAMMVIDNSMTLGMFMAFNAYRGQFSQRASNLIDLAIGLRMLSLHNERISDIVFTDAEPESAPRQIFQPGKGVAVEVKNLTYQYDALSRPIFKDLDMCIAAGESVAVVGVSGAGKTTLLKVMCGLLSPTSGQILADSMDIHKVGVNNYRNAIACVLQDDRLFSGSIAENISGFEVNADKALIVACAVHSNIHDEIMQMPMGYETLIGELGNGISGGQKQRLFIARALYRRPSVLFMDEATSHLDVENELAINRAISSLNITRVIVAHRKSTIDSADRVVVLGAASGDPASNG

SEQ ID NO: 55 - protein, 711 aa, unidentified
  source 1..711: note = Serratia
MKDNNLFERINEKLTFSLRKRVPSILQSESSECGLACLAMIASYYGFNVDMLSLRQRFGISTQGATLGTISQIASQIQLKTRALSLDIDEINQLKTPCILHWNMNHFVVLVKVQRAGFVIHDPAFGRRVIGLQEMSNHFTGIALELWPDRAFQKETLKTRLRLLDLMKNIEGLPGTLLKIFALSIVIESVNLLLPVGTQLVTDHVIQAHDYSLLTVICLGLIFFTLFRAVVSIARAWISIVLGTLTDIQWKTTLFEHLMKLPLDFFEKRHLGDIQSRFSSLDAIRTTFTNNIVSGIIDGIMTVGLFAMMMVYGGWLVWVVAGFTLIYILIRMMTYRTYRQFSEEQIVKAAKANSHFMETLYGISTVKALGIKETRSSYWLNLNVDAANTNIKITRFNMMFGGINTFITTLDQVAILWLGAMMVIDNSMTLGMFMAFNAYRGQFSQRASSLIDLAIGLRMLSLHNERISDIVFTDAETESAPRQVFPSGTGIAIEVKNLTYQYDALSRPIFKDLNMRIAAGESVAVVGASGAGKTTLLKVMCGLLSPTSGQVLADAMDIHKVGVNNYRNAIACVLQDDRLFSGSIAENISGFEVNANKELIMACAIHSNIHDEIMQMPMGYETLIGELGNGISGGQKQRLFIARALYRRPSVLFMDEATSHLDVENESAINRAISSLNITRVIVAHRKSTIDSADRVVVLGAGSGAPAGGGE

SEQ ID NO: 56 - protein, 699 aa, unidentified
  source 1..699: note = Serratia
MTESYLDALKGKLNLSLRRKVPQILQTEASECGLASLAMVCHYHGLQIDLFNLRSRYGFSSRGATLSALIDIASALKLQSRALSLNIDELKALKMPCILHWDMKHFVVLVSVSRGRAVIHDPAFGRKVLGLHELSRHFTGVALELWPDSEFQPIKQQSRLRFRKLMSNVRGLKGALLKIFCLSIVIEAVNLLLPVGTQLVMDHVILAGDHDLLALICIGLLFFILFRTGVSMLRSWISIVMGALIDVQWKAGVFDHLMKLPLSFFEKRKLGDIQSRFGSLDTIRATFTTSIVSSIIDGIMSVGVFIMMLLYGGWLVWVVLGFTAIYVILRLSTYQHYRQLSEEQLVKGARAGSHFMETLYSVSTLKALGLSETRSQYWLNLNVETINAGIKLTKLNMMFGGLGAFIATCDQVIILWLGAALVIDNQMTIGMFIAFNAYRGQFSERASSLIDMMLQLRMLSLHNERVADIVLSSPEPQMPARQLFTKGNAAELQVRNLRYQYDRLAKPIIADLNLSIAAGESVAIVGPSGVGKTTLMKLMCGLLSPDEGAVLVDGMDISNIGVNNYRQCIACVLQDDKLLAGSIAENISGFDAEMDLKRIEACAQRCNMHDDIQNMPMGYETLIGELGGSLSGGQKQRLLIARALYRRPSILFMDEATSHLDLDNETHINRAISQLNITRVIIAHRPSTIASADRVIRLG

SEQ ID NO: 57 - protein, 699 aa, Serratia marcescens
MSKPTFKQRISQLDLRLRHRVPMVHQTESSECGLACLAMICGFYGKNVDLIALRRQFSLSTRGATLTGLTGIAEQLGLATRPLSLDIDELSALKLPCILHWEFNHFVVLVSIKGHRAVLHDPARGRRAISLTELSHSFTGVALEAWPGSAFTADSVRHRIHLRTLIGSVHGLKGALGKIFCLSLVFETINLVMPIGTQLVMDHAIPAGDRGLLTLICVGLMLFILLRAAVGMVRAWSGLVMSTLINVQWQSGLFTHLLRLPLGYFERRKLGDIQSRFGSLDTLRSTFTTSIVGAIMDGIMVIGVLVMMVLYGGWLTWVVIAFTAIYVLMRLLTYGYYRQLSEEALVREARAGSYFMETLYGIATVKMQGMAERRIAHWLNLEIDTINTGIRVTRMDMLFGGINTFIAACDQVVILWLGASLVIDNQMTLGMFVAFGAFRGQFSDRIGSLTEFLLQLRMMSLHNERIADIALHPRENRKPDLPYAAGLQPLGLSTHALSYRYDSQSPAVFDALDISVAPGESVAIVGPSGAGKTTLMKVLCGLFPPDSGRVEVNGVDIRQLGINNYHKMIACVMQDDKLFSGSIRENICGFADEVDDAWMEACARASYLHDVLMRMPMGYETPIGELGEGLSGGQKQRLFIARAIYKKPAILFLDEATSALDKESEAVVNQAIKRLNITKVIIAHRETTIASADRVIHFG

SEQ ID NO: 58 - protein, 699 aa, Serratia marcescens
MSRSTFKQRVSQLDLRLRHRVPMMHQTESSECGLACLAMICGYYGKNIDLIALRRQFNLSSRGATLAGLTGIAEQLRLSTRPLSLDLDELGALKLPCILHWEFNHFVVLVSVKGHRAVLHDPARGRRAVSLTELSHSFTGVALEAWPGSEFTADSVRHRIHLRTLIGSVHGLKGALGKIFCLSLVFETINLVMPIGTQLVMDHAIPAGDRGLLSLICAGLMLFILLRAAIGMVRAWSGLVMATLINVQWQSGLFTHLLRLPLGYFERRKLGDIQSRFGSLDTLRSTFTTSIVGAIMDGIMVIGVLVMMALYGGWLTWVVLTFTALYVLMRLLTYGYYRQLSEEALVREARAGSYFMETLYGIATVKMQGMAERRIAHWLNLEIDTINTGIRVTRMDMLFGGINTFIAACDQIVILWLGTSLVIDNQMTLGMFVAFGAFRGQFSDRIGSLTEFLLQLRMMSLHNERIADIALHPREARKPDHPYTAGLQPLGLTTHALSYRYDSQSSAIFDGLDISVAPGESVAIVGPSGAGKTTLMKVLCGLFTPDSGRVEVNGVDIRQLGINNYHKMIACVMQDDKLFSGSIRENICGFADEVDDAWMQACARASYLHDVILRMPMGYETLIGELGEGLSGGQKQRLFIARAIYKKPAILFLDEATSALDKESEAVVNQAIKRLNITKLIIAHRETTIASADRVIHLG

SEQ ID NO: 59 - protein, 710 aa, Serratia marcescens
MCRGHFVNKVYENIVGKLNLSLRRKVPQVLQTEAAECGLASLAMVCGYYGMHIDMLSMRQKFDISARGATLSSLIAIAENLNLKTRALSLSLDEIHQVKRPCILHWDMNHFVVLVNIRGGRITLHDPAFGRRVIGLQEFSLHFTGIALEVWPSSDFTPVVQKNRLHFRKLLSNVTGLKSALVKIFALSLVIEAVNLLMPVGTQLVMDHVIQAGDHNLLVIICVGLLFFILFRTCVSMFRSWISIVMGALIDIQWKSGLFDHLMKLPLAYFEKRKLGDIQSRFGSLDAIRTTFTTSIVSSIIDGIMSVGVFIMMFMYGGWLVWVASGFTVLYVLLRLSTYRYYRQASEEQLVKSAKASSHFMETLYSIATLKSLGLAGTRSQFWLNLNIDTANANIRVTKLDMFFGGVNAFLAACDQIVILWLGASLVIDSQMTLGMFVAFNAYRGQFSDRASNLIDMVIRLRMLNLHNERLADIVLSTTEEEKPYRKICDPNEAVTFEVRDLLYQYDSLSRPVIPGLSLQIAAGESVAIVGPSGVGKTTLMKLMCGLLEPTEGRLFINGINIHDIGINNYRECIACVLQEDKLLAGSIAENICSFDAHPDTEFIVECAKHCNIHDDIMKMPMGYETLVGELGGSLSGGQKQRVLIARALYRRPSILFMDEATSHLDLDNEKRVNEAISSLKMTRVIIAHRPSTIASADRVITLQPVNAMA

SEQ ID NO: 60 - protein, 699 aa, Serratia plymuthica
MSKFSVKKLLSQLDMRFRHRVPLVHQTESSECGLACLAMICGHYGKNVDLIALRQQFHLSARGTTLSGLTDIAEQLGLSSRPLSLDIDELSALKMPCILHWEFNHFVVLVSIRHNCVVLHDPALGRRIISLVEVSHSFTGVALEVWPSSAFEAHAVHKRLSLATLIGSVHGLRGALGKIFCLSLVIETINLAMPVGTQLVMDHAIPAGDRGLLTLICVGLLLFILLRAAVSMVRAWSSLVMTTLINVQWQTGLFNHLLRLPLGYFERRKLGDIQSRFGSLNALQSTFTASIIGAIMDSIMVIGVLVMMVLYGGWLTWVVIAFTTVYVLLRLLTYGYYRQLSEEALVRNARASSYFMETLYGIATVKMQGMAERRSTHWLNLEIDTINTGIRVTKMDMLFGGINTLVAACDQVVILWLGTSLVIDNHMTIGMFVAFGAFRGQFSDRIGSLTNFLLQLRMISLHNERIADIALHPRENRKPDIHYEARFQPVSLATCALSYRYDSQSPAIFSGLDITIAPGESVAIVGPSGSGKTTLMKVLCGLFPPDSGRVEINSIDIQQLGINNYHKMIACVMQDDKLFSGSIRENICGFADNVDDTWMQECARASYLHEVIIRMPMGYETLIGELGEGLSGGQKQRLFIARAIYKKPAILFLDEATSALDKESEEVVNEAIKGLDITRIIIAHRETTIASADRVIYLD

SEQ ID NO: 61 - protein, 699 aa, Serratia surfactantfaciens
MNKPTFKQRVSQLDLRLRHRVPMVHQTESSECGLACLAMICGYYGKNIDLIALRRQFNLSTRGATLVGLIGIAEQLGLSTRPLSLDLDELGALKLPCILHWEFNHFVVLVSVKGHRAVLHDPARGRRAVSLAELSHSFTGVALEAWPGSAFTADSVRHRIHLRTLIGSVHGLKGALGKIFCLSLVFETINLVVPIGTQLVMDHAIPAGDRGLLTLICVGLMLFILLRAAIGMVRAWAGLVMSTLINVQWQSGLFTHLLRLPLGYFERRKLGDIQSRFGSLDTLRSTFTSSIVGAIMDGIMVIGVLVMMVLYGGWLTWVVIAFTALYVLMRLLTYGYYRQLSEEALVRNARAGSYFMETLYGIATVKMQGMAECRIAHWLNLEIDTINTGIRITKMDMLFGGINTFVAACDQVVILWLGTRLVIDNQMTLGMFVAFGAFRGQFSDRIGSLTEFLLQLRMMSLHNERIADIALHPREDRKPDLPYISKMQPMGLSTHALSYRYDSQSPAVFDALNISVAPGESVAIVGPSGAGKTTLMKVLCGLFPPDSGRVEVNGVDIRQLGINNYHKMIACVMQDDKLFSGSIRENICGFADEMDDAWMEVCARASYLHEVIMRMPMGYETLIGELGEGLSGGQKQRLFIARAIYKKPAILFLDEATSALDKDSEAKVNQAIKKLKITRIIIAHRETTIASADRVIRLD

SEQ ID NO: 62 - protein, 719 aa, unidentified
  source 1..719: note = Yersinia
MIASTMKGYFEALRQRLPVVMQTEATECGLACMAMVAGYYGLNIDLQALRKHYQVSLKGMSLRDLIVLADRLSLGSRPVRADLDAVPQLKTPCILHWSFNHFVVLKKFSRHGAIIHDPAKGERRISITELSQHFTGIALEIWPNQRFQRRTEKQVIRLLDMFKNVSGLSRALAHVLALSFAIELLTMAVPMAIQFTVDMALRSGDINLVTLIVWGIVALLIFRALLSLVRSWTLMSVKYSLGLQWSSGLFSHLLRLPASYFEKRHIGDVSSRFNSLSVVQDAFTAEMVASLLDIVVVIGLLFLMSVYNGYLTAAVVSLSCIYATIKFCLFRAYRSANLEAIAHEAKQHSHFLETVRGINGVKIFNLAERRRSDWLNLVVDEANAKIYLFKIDLATQTVGLLLIGISSAMVLWLGAKLIDVGTMTTGMLFAFLIYSDMYITRTISVVDSIIKLRLIDMHSERLSEVALTPPESDEGEMNLVMPTPLAGTIDVKELSYRYGDGEPAVFENISLSIKAGESIAIVGPSGCGKSTLLKTMGGLAPQESGTILLDGIDVRRLGLEAYRKHIAYVLQEDRLFAGSLLDNISSFDIHPDSEWVFECARLASIHSEIEAMPMKYETMVGDMGSALSGGQRQRISLARALYKRPRILFLDEATSDLDIDNEARINDSIRALKITRIFVAHRPSMIAMADRVFDLSTNTEKEKETPHALFTERKHHVIE

SEQ ID NO: 63 - protein, 704 aa, Yersinia pestis
MNDTHFNEIKNRLNFSFRRKVPQVLQTEAAECGLACLVMTCRYHGMDIDLFNLRQRFGISSHGATLALLINISAQLKFKTRALSLDLDELRQLKTPCILHWDMSHFVVLVAVKGTRFIIHDPAFGRRTVSLSEMSQHFTGVALELWPNSEFTRQKSRTRLSLLSLMRNISGLPGFLTKIFCLSLMVEAINLLLPVGTQLVMDHVIIAEDYDLLALICIGLLFFILFRTFLSMLRSWTSLVMGSLVDVQWKAGLFDHLLKLPLAYFEKRKLGDIQSRFGSLDIIRSTLTNNVVNGIIDGLMSIGVFIMMFLYGGWLVWVVLGFTAMYMILRLATYNQYRQASEEQIVKNAKASSHFMETLYGISTLKALGLAATRSQFWLNLNIDTTNANIRLTKLDMFFGGVNTLIGTIDQVVILWLGASMVIDGQMTLGMFVAFNAYRGQFSDRATNLINMVLQLRMLALHSERIADIVFTETEKEQTPRQLLSPNQPAVFEARNIAFQYDNLSKPIFSDLNIHVEAGESVAITGPSGIGKTTLMKVIAGLLTPSQGHILIDGLDITTVGLNNYRDCIACVLQDDKLFAGSIADNIASFDVNKDEQRILSCANHCNIHKEIMHMPMGYETLISELGGSLSGGQKQRLLIARALYRQPSLLFLDEATSHLDLANEAHINNAIASLKITRIFIAHRPSTIASAQRIINLEKQNVS

SEQ ID NO: 64 - protein, 719 aa, Mixta gaviniae
MKFLEALNFGWRRQLPVMQQTQATECGLTCVGMIANYFGHAIDMVTLRKRFPTSLKGATLADVMLIAHQLGMAGRALRLELDELGKLRRPCILHWEMNHFVVLKSVAKGKITIHDPARGKRDVPMDEVSRSFTGVALELLPAATFTQAQEKQSISMRKLIGNVTGIRSAFAQVMILSVALELFGILSPFYMQWVMDQVLVSANYDLLTLLGCGFIIVTLLNIAISALRSWVTTWFSSLLSVQWTANVCAHLLGLPMAYFESRHVGDVLSRFGSIGTIQNTLTGRFISSILDGVMAVVTLGMLFIYNIRLAWLVLGLLAAYVIIRWISFRPFRQANEDQIAASARAQSQLLESIRGVQAVKLNNKQEMRVAAYANALVESTNKGVAIQRLSIGFSSVQGAISGVGRIVLIWMAALQVLEGNFTSGMLVAFISFSDQFISRASGLVDAVIDFTMLRLHGERLADIVLTDREADMETLLPAPMHAAQSAPSLTIKDLSFRYAETEPYVLRHCDIAVAAGESVAIIGPSGQGKTTLAKLMMGLLKPESGCIELDGVDIKKLGMRHYRARLGCVMQDDILFAGSLSDNICFFDAQPDQEKIEDAAKVAQIHDDISAMPMGYHSLVGDMGSSLSGGQIQRVLLARALYRQPDLLILDEASSHLDVDRERMINQAIRHMPVTRIIIAHRPETICSADRIILLNQGKAVEIDKAHYARLSNTSPQSR

SEQ ID NO: 65 - protein, 705 aa, Acinetobacter baumannii
MRYLDRLSLGLGRKVPVILQTESSECGLACLAMIAGYYGYESDLLTLRQKYPISQKGATLTTLVKIANKLHLTTRPLKLELDELNQLRLPCILHWDLNHFVVLKSVSATKITIVDPAFGEKVFTYDQASSHFTGIALELWPDSNFEEKKDKTSIRIFKLFGEIRGLWKSLGQILILALVLEVFSLVSPFFMQWVIDHAIVSADLNLLTTLAIGFGLLMILSSLISLLQAWVVMHMATTLNVQWKANIFHHLVNLPTNFFQKRHLGDIISRFGSIDAIQRTLTTSFITAILDGLMTIFTLILMFLYSTKLAWIAVITMLLYAVIRWIWYYPLRRATEDQIVHAAKQNTHFMETMRGIKTIKQFEKQDYRQATWLSLFVDQINAGLTTQKLNLMFGFVNTLLFGLQNIIIVWLGATLVIEGEFTVGILMAFLAYKNQFGGRVSSLIDKYVEVKMLSLHGERLADIVLTDQEKTDSYSFVPELSEQQKTGIDVKSLRFRYSEDESWIIHGINFNIPEGQSVAIVGPTGCGKTTLMNLLLGNLTPEHGEIKIGGQSIKNIGSNKLRNFIAYVAQDDVLFAGSILENISFFDDGAQQDWVEQCAKMASVHDEIMAMPMGYQTLVGDMGNILSGGQKQRILLARALYRKPKILFLDEATSHLDIIKEKEINNMIKSLNITRIIIAHRPETISSVDRIIALNNGQIVSDQML

SEQ ID NO: 66 - protein, 708 aa, Acinetobacter baumannii
MRYLERLSLGLGRTVPVILQTESSECGLACLAMIAGYYGYESDLLTLRQKHPISQKGATLATLVKIANKLHLTTRPLKLELDELNQLRLPCILHWDLNHFVILKNVSPAKITIVDPAFGERVFTYDQASSHFTGIALELWPDSNFEEKKDKTSIRIFKLFGEIRGLWKSLGQILILALVLEVFSLVSPFFMQWVIDHAIVSADLNLLTTLALGFGLLMLLSSLISLLQAWVVMHMATTLNVQWKANIFHHLVNLPTNFFQKRHLGDVISRFSSIDAIQRTLTTSFITAILDGLMTIFTLILMFIYSVKLACIALISMLMYIIIRWIWYYPLRRATEDQIVHGAKQSTHFMETMRGIKTIKQFEKQDYRQATWLSLFVNQINAGLTTQKLNIMFGFVNTLLFGLQNIIIVWLGATLVIEGEFTVGILMAFIAYKNQFGDRVTSLIDKYVEVRMLSLHGERLADIVLTDQEKTDNYLFVPESSELQKTEIDVKSLRFRYSEEESWIIHGINFNIPEGQSVAIVGSTGCGKTTLMNLLLGNLNPEHGEIKIGGQSIKSLGSNRLRNFIAYVAQDDVLFAGFILENISFFDDSVQQDWVEQCAKMASVHDEIMAMPMGYQTLVGDMGNILSGGQKQRILLARALYRKPKILFLDEATSHLDIIKEKEINDMIKSLNITRIIIAHRPETISSVDRIIALNNGRIVSDQMYEPL

SEQ ID NO: 67 - protein, 719 aa, Enterobacter sp.
MDFIEKIRFSARKSLPLLRQTQAAECGLACIGMIAGYYGYKMDMLTLRKKYAMSLKGSTLTDIINLAGHLGLVTRALRLEVEELSQLRLPAILHWDMKHYIVLSEIHGDKVTIHDPARGRRILKIKDLAASFTGIALELVPQSDFEKREEKESLSMLKLLGNISGIKSVFAQLMILTLSLEIFGIITPFYMQWVLDQVLVSSDYNLMTLLGCAFIIVVLLQNGITALRTWVTTWFSSLLSVQWSSNICSHLLNLPQSWFEERHMGDIVSRFGSINNIQSTLTTRFISSVFDGVMSIITLGIIVVYSPSLSLIVVGLFLAYTTVRMVAFQPFRQASEDQMVANSQVQSQLIESIRGSQAIMLSNKQNMRVSTYTNELVEATNNGIKIQRLSIFFTTVQGILSGVGRIVLIWLAALQVLDGHFSSGMLMSFITFSDQFISRASGLIDAFIEFRMLRLHGERLSDIVLSERETDMLNLSAHPDPIHAVSVEFKNLSFRYSPTEPWILKQVNMLIHAGESVAIVGPSGQGKTTLARLLLGLLRAEEGAIIIDGVENVRLGMTWVRNNTGSVMQDDQLFAGSILDNICFFDADFDMSMVEEAAKIAQVHTDIIAMPMGYNSLVGDMGSALSGGQIQRLLLARALYKKPSILLLDEATSHLDIYREKAINEAIRKMSITRIIIAHRPETIMSADRIFVVANHKINEVDKASLFGIQNGEMKPALT

SEQ ID NO: 68 - protein, 707 aa, Escherichia albertii
MYMSKLSFSIKKNFPFFYQNADKDCALACIKMLASYYKKEHLLNCLSQQAHNKKTFWSVNDILSALRTLGMNPRPVRLELDEINSLSIPSVLHWNMNHFVLLVGKKKNKLIIHDPEKGIRHLSLKELSSSFTGVAVEVYNDKIISEKKTSIKDNKVMHYFFMAISLGRKNFIYLFILILLSEIISITLPQITQLVLDNVIINSDYQLLLTAVTFYLFLHVINTGILATRDWLIIWLNANINAQWGINFYNRLVLLKRNYFSSRSTGDILSRFSSLEYIRNVIISKTTTSLLDFFMAIGSLIIMLTYSLNLTFIVIFSTGVYFLLKILYFGSVKHFNLGTIRIKAQQQSSLIEAVKYNQTIKLYFDEYMSAGKYVGELIEGVNVKTQIDILNIIFSASNRILTALKNICILYFGGVFVIKTSFTIGMLVAFITYSEQFSRRVTSLIDFFVQIGITRLHVSRVSDISDADYETISADMPKRISSPLQVEFNNVSVKHPDKQINILNRINFKIMPGETVILKGISGCGKTTLLNAILGLIDVSSGHISITENGMKKSCIDARKMTGAVLQGDSLLNGTVIYNITFNDSSPSRDVIDLTKMIGVHDVIKSLPMGYHTQISDSDHILSAGQKQKILLARAMYRKPQLLILDEATSNLDIDSEYQISQAILSLNCTKIIVSHKEKSFFMADKVITIKNGMIVNIHEVIKNQHL

SEQ ID NO: 69 - protein, 706 aa, Halomonas elongata
MNLSELPKRLLGGIRRGTPVIIQSEAAECGLACMAMIASHHGHALDLPAMRHRFSTSLKGMALRDLLKVGGDLKLSTRAVRLELSDLGKLQLPCVLHWNHNHFVVLTRVGTNSVTIHDPATGSRKLPFSEVSKRFTGVALEAWPNELFTKKNERERIHVTEMIKRTSGIGRAALQIIAISVLLEIVTIAMPIGFQMVIDQVIVAQDYDLLTLITIAFVLLLILQVLASFVRSWSSMLIGSSLVLQWKAGLFDHLMRLSLSYFEKRHVGDIVSRFGSLDSIQKTLTTSAIMALLDGVMSIALIVMLWLYGGWLASVPLIAVLLYTIIRLCLYHPYRNLSEEAIVYAAQENTHFMESLRGMASLKVLNLEERRRSVWINYLVDRVNANLQVEKFDIVFQNASRFLFGVDRIIIIYFGARAIMGDALSVGMLIAFLSYKDQFTNRMDNFVSTMIKLRMLSLHGERVADIALIEPEKSDLSSGSTESEREEEGAGSIDVKNVSFRYADSEENVLKDVSFSIDAQECVGISGPSGSGKTTLLKILSGLMLPSEGHVCIDGKELSSIGAAAYRDRVGCVLQDDRLFAGSVAENISGFDPDPDTRRIHQCAMLAAIHDEIAKMPMGYETFVGDMGSTLSGGQMQRIILARALYRRPKVLLLDEATSHLDPENEAAINAAIQKLSITRVVIAHRPSTLAMTDRVIDMQALQGGA

SEQ ID NO: 70 - protein, 723 aa, Limnobaculum parvum
MSFVKKINFGLTSQRLPVLIQTESAECGLACLAMIAGYHNYNIDLATLRGKYSISQKGANLSQLLNISKQMNLSSRSLRLEMEDLPKLKLPCILHWEFSHFVVLKAIKGNKCIIHDPAVGMRQLAFTEVSKSFTGVAVELWPDDGFKPQEVRRQVRFRDLIGKITGLKSSFLQVLLLALGIELFSIISPLFTQWTIDYVIVSNDRNLLVTLIVGFGVMMLVQQLTSAVRAWVMMYISTSISVQWRDSVFHHLLRLPISYFEKRHLGDIVSRFGAVDAIQSTLSSSFFVAILDGLMTIVTLVMMYIYSPYLATICLITMVLYIIGRIFWYRPLRMATEEQIIHGAKQQSHFLETIRGVRTIKLFQRQNERMSDWMALLISQINSGVRVQKLQIYYQQLNGLLFGIENLVVLGVGATMVMDQTFTVGVLMAFSSYKGQFSSRISSLVDKFFELKMLSIQTERLADIVLTEPEDDIEQKQYVQQTQPPRIEFDNVSFRYSEHEPYVIKDTSFVIESGECVALAGASGCGKTTLSQLLCGSLVPVSGDILIDGVSLKQLTVANLRRFSATVLQDDVLFAGSIMDNITFFDHNINHEKAIKCAMMAAIHHDIEQMPMQYNTLVGDMGVALSGGQKQRILLARALYKQPQLLILDEATSHLDVQLEQLVNTAVSQLAITRLVIAHRPQTLAMVDRIIVIENGTVGHDLTPAQLFSSAAPSQGSAAEQGS

SEQ ID NO: 71 - protein, 707 aa, Methylomonas sp.
MQHILDFSGRRKTPLILQTEAAECGLACLAMVANFHGHRVDLASLRQKFSLSLKGSTLNHLIQIANQLHLGSRPVRVDLNELSKLSLPAILHWDFNHFVVLTQVKRGVATIHDPAKGTVNLPFTEVSKHFTGIALELQPTQAFTAKTERQQIKLSRLIGQLNGASSATLQIILLAAAIEVFAIVAPFFMQLVVDNAVVARDTNLLTVLGLGFLLLALIQIGITALRSWVVLVLSTSVNLQMMSNLFGHLLQLPMNFFEKRHLGDIVSRFESLNVIQRTLTTSFLEAIIDGVMALVTLGMMLVYSWKLAAIVSLAALLYGLLRLVSFAPLRAASEEQILRAAKQQTNLLETVRGMQSVKLFNRQLQRRHGYQNLMVDHFNAGIRVQKLNIIYRALNSLLFGVENIAVIWLGALFILDGGFSVGMLFAFMSFKDQFTHRVGSFIEKGIEFKMLGLHTERVADIALAEPEQEHCSGARQDQLPASIQIRNLDFSYAPSEPPVLKNLNLDFAEGESVAIIGPSGYGKTTLAKLILGLLQPSAGEILIGGVRLNRIDSHDYRNMVAAVMQEDQLFAGSIADNICFFDPEPDQAWIETCAQLAAVHQDVMTMPMAYNTLIGDMGTVLSGGQKQRVLLARALYKRPKILVLDEATSHLDVAREKLVNGAVQQLKLTRIIIAHRPETIASVDRVIDLQAIADAALGEQHYVSQAA

SEQ ID NO: 72 - protein, 727 aa, Pantoea vagans
MLLLEKLNFKWFNRLPMIRQSQAAECGLACLGMVANYHGHQIDMITLRRQFATSLKGATLADVIAIAQQLNMTSRALRVELEELSKLRMPCILHWELNHFVVLKKVRGNKITIHDPARGIRQLTFKEASTAFTGVALELVPSSTFEVKEEKESISMMKLVGSVTGVKSAFAQVLILSIALELFGVLGPFFMQWVMDMVLVSADYSLLSLLGVGFIMIALFQTIVTALRSWVMNWFSSQLSVQWTINVCHHMLKLPLEWFESRHVGDILSRYGSLNTIQSTLTSRFISTVLDGVMSIVTVVMLFIYNAQLAWLVIGLFLAYALLRFMAYDPVRRANEEQIISSARTQSSLLETLRGIQAVKTNNKQVPRLSAYMNFLVDTTNKGIVIQRLNILFGSAQGLLTSVGRVVLVWLAALQVLDGNFSAGMLTAFISFSDQFMSRGSGLINAIIDFRMLRMHGERLADIVLSETEASSEGNPGLVSKETDKETDVPQDIKLINIRFRYAPTEPWIVDGANLEIKAGESLAIVGPSGQGKTTMAKIILGLLHPEEGTITVGGMDITQTGLEHHRNRIGCVMQDDILFSGSISENISFFDNEPDHAKITRVARLAQIHGDIMKMPMNYQSLVGDMGSFLSGGQLQRILLARALYREPKILVLDEATSHLDIYNEAQINNAIKQMKITRIIIAHRPETIRSADKIVLLNNGTLSEVTAEQLFGQAATHNETEITYG

SEQ ID NO: 73 - protein, 714 aa, Pseudoalteromonas rubra
MHVEDESPVQQLQFWSRKSLPVILQSEAAECGLASLAMVAAYHGYHSDLTSLRQKFSISIEGATLLDIMHFAEKLEMSSRPLRIELEDLDALQTPCILHWDMNHFVVLKKANEKRIVIHDPASGEKTYTMAEASKHFTGVALELTPTKSFEKKEKKPSLQFSDFWSRITGLKRSLTLIFLLSILLQIFTLAAPYYIQLVIDDVVLTGDTNLLTVLATGFFLVLVFEIATNALRGFTLLHFGNQMNIQLGANLFHHLVRLPISYFEKRHMGDVVSRFGSLQQVKQILTTGVIEAIIDGLMAIITLAMIFFYSPTLSAVVLAAVVAYAIVRIAMYRPFRNISEQEIMARAEENSNFMETVRGIQTIKLFGSEVKREGQWQNRYANAINQSIRLGNFQIGYDAINRALFGIENILVVYLAAHLVLDGGFSTGMLFAFMSYKRQFMDKTANLIEKLIEFKMVGLHFDRIADIALTDKEILQPDQVKQHQVKGQIELRNISFSYSDATPNVLNDLNLTIEAGESVAITGPSGCGKSTLLKIMLGLNLPKSGEILIDGVPMEQIGARQYRQQIAAVMQDDELLSGSVADNIAFFDTPIDMERVVYCAQLAAIHDDISQMPMNYDSLIGDMGSSLSGGQKQRIILARALYKQPKILFMDEATSHLDTNLESDINEAVSRLDITRVIIAHRKETIASADREVRLKKPDIPDPYAQEEESVCE

SEQ ID NO: 74 - protein, 719 aa, Pseudomonas sp.
MAFLDALALRLGRRLPLVLQTEATECGLACLAMIAGYHGHHTGLMELRRRFSVSLKGISLKQLIQTAHRLGLGTRAVKLDLGDLGKLKLPCVLHWNFNHFVVLKAVDGRGAVLHDPAHGQRRLGLEEVSRSFTGVALELWPESGFEKQEAPPRIKLLGMLGKVTGLYRSLAQVLLLAGALEVFSLISPFFLQWTIDNVIVSEDRDLLSTLAIGFGLLLLMQQAVSGVRAWVMMHMSTLLGVQWQANVFSHLLRLPAQYFEKRHLGDVVSRFGAVNSIQQTLTAAFLSAVLDGLMTVATLGMMLLYSPPLAAIAIAAMSLYALGRWIWYRPLRNATEEQIVHAARQQSHFLETVRGIRPLKLFQRQDERRSVWLGLLVEQINAGLRTQKLQLFYQQLNGLLFGVENLLVIWLGATMVMDGQFSVGILMAFNAYKSQFDSRVGSLIDKFFELRMLQLQGERLADIVLQAPEVSHGDILPENLREREASIEIQGLRYRYAEQEPWVLDGLDLRIAGGESVAIVGPSGCGKSTLLNVLLGILPPVEGQIRMAGLDLAQLGLDGLRELVGTVLQDDVLFAGSLSDNISFFDPQPDMPWLLQCAQMAAIHDDIQAMPMGYNTLVGDMGTVLSGGQKQRVMLARALYKKPRILFLDEATSHLDVHCEQRVNAAIRALRITRIMVAHRPETIASADRVIVLGQGKVSLDESTARLAERQAAAAREQA

SEQ ID NO: 75 - protein, 719 aa, Pseudomonas sp.
MAFLDALALRLGRRLPLVLQTEATECGLACLAMIAGYHGHHTGLMELRRRFSVSLKGISLKQLIQTAHRLGLGTRAVKLDLGDLGKLKLPCVLHWNFNHFVVLKAVDGRGAVLHDPAHGQRRLGLEEVSRSFTGVALELWPESGFEKQEAPPRIKLLGMLGKVTGLYRSLAQVLLLAGALEVFSLISPFFLQWTIDNVIVSEDRDLLSTLAIGFGLLLLMQQAVSGVRAWVMMHMSTLLGVQWQANVFSHLLRLPAQYFEKRHLGDVVSRFGAVNSIQQTLTAAFLSAVLDGLMTVATLGMMLLYSPPLAAIAIAAMSLYALGRWIWYRPLRNATEEQIVHAARQQSHFLETVRGIRPLKLFQRQDERRSVWLGLLVEQINAGLRTQKLQLFYQQLNGLLFGVENLLVIWLGATMVMDGQFSVGILMAFNAYKSQFDSRVGSLIDKFFELRMLQLQGERLADIVLQAPEVSHGDILPENLREREASIEIQGLRYRYAEQEPWVLDGLDLRIAGGESVAIVGPSGCGKSTLFNVLLGILPPVEGQIRMAGLDLAQLGLDGLRELVGTVLQDDVLFAGSLSDNISFFDPQPDMPWLLQCAQMAAIHDDIQAMPMGYNTLVGDMGTVLSGGQKQRVMLARALYKKPRILFLDEATSHLDVHCEQRVNAAIRALRITRIMVAHRPETIASADRVIVLGQGKVSLDESTARLAERQAAAAREQA

SEQ ID NO: 76 - protein, 719 aa, Pseudomonas sp.
MAFLDALALRLGRRLPLVLQTEATECGLACLAMIAGYHGHHTGLMELRRRFSVSLKGISLKQLIQTAHRLGLGTRAVKLDLGDLGKLKLPCVLHWNFNHFVVLKAVDGRGAVLHDPAHGQRRLGLEEVSRSFTGVALEFWPESGFEKQEAPPRIKLLGMLGKVTGLYRSLAQVLLLAGALEVFSLISPFFLQWTIDNVIVSEDRDLLSTLAIGFGLLLLMQQAVSGVRAWVMMHMSTLLGVQWQANVFSHLLRLPAQYFEKRHLGDVVSRFGAVNSIQQTLTAAFLSAVLDGLMTVATLGMMLLYSPPLAAIAIAAMSLYALGRWIWYRPLRNATEEQIVHAARQQSHFLETVRGIRPLKLFQRQDERRSVWLGLLVEQINAGLRTQKLQLFYQQLNGLLFGVENLLVIWLGATMVMDGQFSVGILMAFNAYKSQFDSRVGSLIDKFFELRMLQLQGERLADIVLQAPEVSHGDILPENLRDREASIEIQGLRYRYAEQEPWVLDGLDLRIAGGESVAIVGPSGCGKSTLLNVLLGILPPVGGQIRMAGLDLAQLGLDGLRELVGTVLQDDVLFAGSLSDNISFFDPQPDMPWLLQCAQMAAIHDDIQAMPMGYNTLVGDMGTVLSGGQKQRVMLARALYKKPRILFLDEATSHLDVHCEQRVNAAIRALRITRIMVAHRPETIASADRVIVLGQGKVSLDESTARLAERQAAAAREQA

SEQ ID NO: 77 - protein, 719 aa, Pseudomonas sp.
MAFLDALALRLGRRLPLVLQTEATECGLACLAMIAGYHGHHTGLMELRRRFSVSLKGISLKQLIQTAHRLGLGTRAVKLDLGDLGKLKLPCVLHWNFNHFVVLKAVDGRGAVLHDPAHGQRRLGLEEVSRSFTGVALELWPESGFEKQEAPPRIKLLGMLGKVTGLYRSLAQVLLLAGALEVFSLISPFFLQWTIDNVIVSEDRDLLSTLAIGFGLLLLMQQAVSGVRAWVMMHMSTLLGVQWQANVFSHLLRLPAQYFEKRHLGDVVSRFGAVNSIQQTLTAAFLSAVLDGLMTVATLGMMLLYSPPLAAIAIAAMSLYALGRWIWYRPLRNATEEQIVHAARQQSHFLETVRGIRPLKLFQRQDERRSVWLGLLVEQINAGLRTQKLQLFYQQLNGLLFGVENLLVIWLGATMVMDGQFSVGILMAFNAYKSQFDSRVGSLIDKFFELRMLQLQGERLADIVLQAPEVSHGDILPENLREREASIEIQGLRYRYAEQEPWVLDGLDLRIAGGESVAIVGPSGCGKSTLLNVLLGILPPVEGQIRMAGLDLAQLGLDGLRELVGTVLQDDVLFAGSLSDNISFFDPQPDMPWLLQCAQMAAIHDDIQAMPMGYNTLVGDMGTVLSGGQKQRVMLARALYKKPRILFLDEATSHLDVHCEQRVNAAIRALRITRVMVAHRPETIASADRVIVLGQGKVSLDESTARLAERQAAAAREQA

SEQ ID NO: 78 - protein, 712 aa, Pseudomonas sp.
MNLRNTFAFGVGRKLPVVLQAEATECGLACMAMIASYHGQHSDLLSLRQRLSPSMKGVNLKQLTHMAARLGLGSRALRVELNALGQLQLPCVLHWNFNHFVVLKEVNARGVVVHDPGRGLCKLTLDEVSAAFTGVALELWPESDFQPAAAQPPIPLRKLLGRVQGFGRVLTQVLLLALALELCTILSPFFMQTVIDKVLVSADLDLLTVLAIGFGLLLIIQHTVALARAWALMYLGTMLGSQWQINVFTHLLRLPVAFFERRHLGDIVSRFGSLKAIQQTVTTSFIEALLDGLMTVVTLGLMIAYSAKLALVAVVAMIIYGLARWIWFGPLRRASENQLVHAARQQSHFLESARGVRTIKLFNRHEQRCSAWSALLVEEINAGLHPQKLQLAYRAFNGVLFGLVTIIVIWMGARLVLEGQFSAGMLIAFNSYKEQFNGRVAGLIDKIVDVIMLRLHGERLADIVLHDAEPIAGPDTFDDACNDRVPSLEVRQLRFRYSEHEPYVLDDVSIHIRAGESVAIVGPSGGGKSTLLNVMLGILAPCAGSVLLDGLPVGPENIGRLRRITGTVLQDDVLFAGSIGDNISFFETGADQKWIEQCAHLAAVHDDIAQMPMGYNTLVGDMGTVLSGGQKQRVLLARALYKRPQLLFLDEATSHLDINREAAVNQALQTLNITRIIVAHRPETIRTADRVVALAQGRVAYDGPVLNEAPSA

SEQ ID NO: 79 - protein, 719 aa, Pseudomonas aeruginosa
MAFLDALALRLGRRLPLVLQTEATECGLACLAMIAGYHGHHTGLMELRRRFSVSLKGISLKQLIQTAHRLGLGTRAVKLDLGDLGKLKLPCVLHWNFNHFVVLKAVDGRGAVLHDPAHGQRRLGLEEVSQSFTGVALELWPESGFEKQEAPPRIKLLGMLGKVTGLYRSLAQVLLLAGALEVFSLISPFFLQWTIDNVIVSEDRDLLSTLAIGFGLLLLMQQAVSGVRAWVMMHMSTLLGVQWQANVFSHLLRLPAQYFEKRHLGDVVSRFGAVNSIQQTLTAAFLSAVLDGLMTVATLGMMLLYSPPLAAIAIAAMSLYALGRWIWYRPLRNATEEQIVHAARQQSHFLETVRGIRPLKLFQRQDERRSVWLGLLVEQINAGLRTQKLQLFYQQLNGLLFGVENLLVIWLGATMVMDGQFSVGILMAFNAYKSQFDSRVGSLIDKFFELRMLQLQGERLADIVLQAPEVSHGDILPENLREREASIEIQGLRYRYAEQEPWVLDGLDLRIAGGESVAIVGPSGCGKSTLFNVLLGILPPVEGQIRMAGLDLAQLGLDGLRELVGTVLQDDVLFAGSLSDNISFFDPQPDMPWLLQCAQMAAIHDDIQAMPMGYNTLVGDMGTVLSGGQKQRVMLARALYKKPRILFLDEATSHLDVHCEQRVNAAIRALRITRIMVAHRPETIASADRVIVLGQGKVSLDESTARLAERQAAAAREQA

SEQ ID NO: 80 - protein, 719 aa, Pseudomonas aeruginosa
MAFLDALALRLGRRLPLVLQTEATECGLACLAMIAGYHGHHTGLMELRRRFSVSLKGISLKQLIQTAHRLGLGTRAVKLDLGDLGKLKLPCVLHWNFNHFVVLKAVDGRGAVLHDPAHGQRRLGLEEVSRSFTGVALELWPESGFEKQEAPPRIKLLGMLGKVTGLYRSLAQVLLLAGALEVFSLISPFFLQWTIDNVIVSEDRDLLSTLAIGFGLLLLMQQAVSGVRAWVMMHMSTLLGVQWQANVFSHLLRLPAQYFEKRHLGDVVSRFGAVNSIQQTLTAAFLSAVLDGLMTVATLGMMLLYSPPLAAIAIAAMSLYALGRWIWYRPLRNATEEQIVHAARQQSHFLETVRGIRPLKLFQRQDERRSVWLGLLVEQINAGLRTQKLQLFYQQLNGLLFGVENLLVIWLGATMVMDGQFSVGILMAFNAYKSQFDSRVGSLIDKFFELRMLQLQGERLADIVLQAPEVSHGDILPENLREREASIEIQGLRYRYAEQEPWVLDGLDLRIAGGESVAIVGPSGCGKSTLFNVLLGILPPVEGQIRMAGLDLTQLGLDGLRELVGTVLQDDVLFAGSLSDNISFFDPQPDMPWLLQCAQMAAIHDDIQAMPMGYNTLVGDMGTVLSGGQKQRVMLARALYKKPRILFLDEATSHLDVHCEQRVNAAIRALRITRIMVAHRPETIASADRVIVLGQGKVSLDESTARLAERQAAAAREQA

SEQ ID NO: 81 - protein, 719 aa, Pseudomonas aeruginosa
MAFLDTLALRLGRRLPLVLQTEATECGLACLAMIAGYHGHHTGLMELRRRFSVSLKGISLKQLIHTAHRLGLGSRAVKLELADLGKLKLPCVLHWNFNHFVVLKAVDGRGVVLHDPAHGLRRLGLDEVSRSFTGVALELWPESGFEKQEAPPRIRLLGMLGKVTGLYRSLAQVLLLAGALEVFSLVSPFFLQWTIDNVIVSEDRDLLSTLAIGFGLLLLMQQAVSGVRAWVMMHMSTLLGVQWQANVFSHLLRLPAQYFEKRHLGDVVSRFGAVNSIQQTLTAAFLSAVLDGLMTLATLAMMLLYSPPLAAIAIAAMSLYALGRWIWYRPLRNATEEQIVHAARQQSHFLETVRGIRPLKLFQRQDERRSVWLGLLVEQINAGLRTQKLQLFYQQLNGLLFGVENLLVIWLGATMVMDGQFSVGILMAFNAYKSQFDGRVGSLIDKFFELRMLQLQGERLADIVLQAPEANHGDTLPDNLREREASIEIQGLRYRYSDQEPWVLDGLDLRIAGGESVAIVGPSGCGKSTLFNVLLGILPPVEGQIRMAGLELSQLGLDGLRELVGTVLQDDVLFAGSLSDNISFFDPQPDLPWLLQCAQMAAIHDDIQAMPMGYNTLVGDMGTVLSGGQKQRVMLARALYKKPRILFLDEATSHLDVHCEQRVNAAIRALRITRIMVAHRPETIASADRVVVLGQGKVTLDESTARLAERQAAAAREQA

SEQ ID NO: 82 - protein, 712 aa, Pseudomonas amygdali
MNLRNTFAFGVGRKLPVVLQAEATECGLACMAMIASYHGQHSDLLSLRQRLSPSMKGVNLKQLTHMAARLGLGSRALRVELNALGQLQLPCVLHWNFNHFVVLKEVNARGVVVHDPGHGLCKLTLDEVSAAFTGVALELWPENDFQPAAAQPPIPLRKLLGRVQGFGRVLTQVLLLALALELCTILSPFFMQTVIDKVLVSADLDLLTVLAIGFGLLLIIQHTVALARAWALMYLGTMLGSQWQINVFTHLLRLPVAFFERRHLGDIVSRFGSLKAIQQTVTTSFIEALLDGLMTVVTLGLMIAYSPKLALVAVVAMIIYGLARWIWFGPLRRASENQLVHAARQQSHFLESARGVRTIKLFNRHEQRCSAWSALLVEEINAGLHPQKLQLAYRAFNGVLFGLVTIIVIWMGARLVLEGQFSAGMLIAFNSYKEQFNGRVAGLIDKIVDVIMLRLHGERLADIVLHDAEPIAGPDTFDDACNDRVPSLEVRQLRFRYSEHEPYVLDDVSIHIHAGESVAIVGPSGGGKSTLLNVMLGILAPCAGSVLLDGLPVGPENIGRLRRITGTVLQDDVLFAGSIGDNISFFETGADQKWIEQCAHLAAVHDDIAQMPMGYNTLVGDMGTVLSGGQKQRVLLARALYKRPQLLFLDEATSHLDINREAAVNQALQTLNITRIIVAHRPETIRTADRVVALAQGRVAYDGPVLNEAPSA

SEQ ID NO: 83 - protein, 727 aa, Pseudomonas donghuensis
MNIRQSLAFGIGRKLPMVLQAEAAECGQACLAMIAGYHGQHHDLFSLRQKLSPSMKGATLKQLMAMAAQLGLASRPLRLELQALGQLRLPCVLHWNFNHFVVLKEVGPRGVTVHDPGRGVCKLSFDEVSTAFTGVALELWPQGDFQPGPANPPLRLRQLLGRVQGFAGVLSHVLLLAAALELCIVLSPFFLQTVIDKVLVSADLDLLAVLAIGFGLLLLMQQTLALARSWALMYLGTLLSSQWQINVFSHLVRLPVAFFERRHLGDIVSRFGSLKSIQHTLTTSFVEALLDGLMTLVTLGLMFAYSPLLALIALLAMVIYALARWAWFGPLRRASEEQLVHAARQQSYFLESMRGVRTIKLFGHQEQRASTWGSLLVEEINAGLRPQKLELAYRAFNGLLFGLVTLLVIWLGARLVLEGQFSAGMLIAFNAYKEQFNSRVAGLIDKLVDVLMLRLHGERLADILLQVPEHRQFPAAGDSDADECVPSLEVRQLKFRYGEHEPYVLDEVSVRIEAGDSLAIVGPSGGGKTTLLNVMLGILTPSSGSVLLDGVAIDSHNLDRLRRISATVLQDDVLFAGSIADNISFFSAEANPRWVEQCARMAAVHDDIAQMPMAYNTLVGDMGTVLSGGQKQRILLARALYRRPKLLFLDEATSHLDIAREAAVNQALQALNITRIIVAHRPDTIRSARRVLALDNGRVVYDGPLIETDEHAPAESGEIIRGAASSS

SEQ ID NO: 84 - protein, 717 aa, Pseudomonas sp.
MNYIDAITPSLGRRLPLILQTEATECGLACLAMIAGMHGHHTTLMELRRQFSVSLKGITLRQLMQTAERLNLGTRAVRLELSDLNKLKLPCVLHWNFNHFVVLKAVHGQHLTLHDPGQGVRQLSFAQASECFTGVALEVWPEQGFAQQAAKPRLGLTKMLGRVSGLYRGLAQVLLLALVLEVFSLVSPFLLQWTLDNVVVTQDRDLLTTLMIGFGLLLLMQQLVSATRAWVMMHMSTLLSVQWRANLFTHLLRLPIQYFEKRHLGDVVSRFGAVDQIQHTLTAAFFSAILDGLMTVATLVLMFLYSPSLAMIAVLAMVAYILVRWAWYGPLRRASEEQIVHAARQQSHFLETLRGVRTLKLFQRQGERRSTWLGLLVAQINAGLRTQKLQLLYTQLNALIFGLENLCTIGLGASLVMDNHFTVGVLMAFMAYKGQFVSRVASLVDHLFELHMLRLQGERLADIVMHPPEEQRDAPQSVDWPARQMTIDIQGLSYRYSEHEPLVLDGIDLRIAPGESVAITGASGCGKSTLVNVLLGILPPTSGQIRMGGHELRQLDMASWRGRIGTVMQDDCLFAGSLADNITFFESSPDLEWAMECARMAAIHAEIMAMPMGYNTLVGDMGTVLSGGQKQRLLLARALYKRPDILILDEATSHLDAASEQRVNAAVRALAVTRIIVAHRAETIASADRVVRLEAGRMVLDQRVASVSSYLDEAVTP

SEQ ID NO: 85 - protein, 707 aa, Pseudomonas sp.
MAQLRFDSRSKLPIIMQQEKSECGFACIAMIASFYGHEISLREVRAMHRGSSRGATLLEIVKVAMQLGMQARPLKIENNEIDSLKLPALLHWKNSHYVVLRELRAGKFCIHDPASGVVNLSREEFSIFFTGAALELAPGHQFQLKNKIPGVAVRDILGKVHGLHEALVGILASALALEAFSFITPLFTQLVIDKAISSADLDLLLVFGMSFCVLIIFQSFISYLRDWLVCRTGSTLNQSWTGGVFAHLMSLPEDYFQRRSLGDIGSRFGSLKNIQQAITSTITTSFLDGFMSLVTLAAMVIYSPKLATVAFVFTSAYVLVRLALYSKFKSLNIDLISAQARQNSKFYEAVRASQTIKANNLTAQVTTTYLNLVTSYLNRNILASRLTIAFKAFADLIQGIEKIVLLWIGATLVIQQELTAGMLMSISMFSLLFGSKIAHLADNYVELKLLKIHAARISDIVHTEKELHYYPTFFGEIKDKTIEFASVYFRYSPSEPWILENCCFTIKPGEAVAITGSSGIGKSTILKLICGLLEPNSGKILIGGVDVTTIGKEQLRSWMAVILQDDTLFSGTIAENISLFNEQYSIEEISTAAQEACIHGEIIKMPMGYNTLIGDMGSSLSGGQRQRLLIARSLIRTPSFLLFDEATSHLDIENEFRISEILAKRNCTRIIVAHREATIKLCNRILHMEGRRLKDKTNKTITPHLVV

SEQ ID NO: 86 - protein, 716 aa, Pseudomonas syringae
MNLKNAFTFGVGTKLPVVLQTEATECGLACMAMIARYHGQHSDLFSLRKRLSPSMKGVNLKQLISMAAQLGLGSRALRVELSALGQLQLPCVLHWNFNHFVVLREVTAYGVVLHDPARGLCKLSLDEVSAAFTGVALEVWPESDFQPVAARPPISLRKLLGRVHGFGRVLTHVLLLALALELCTILSPFFMQTVIDKVLVSADLDLLAVLAIGFGILLIMQHTVALARGWALMYLSTMLGAQWQINVFTHLLRLPVAFFERRHLGDIVSRFGSLKSIQQTVTTSFIEAILDGLMTVITLGLMIAYSPKLALVAVVAMVIYALARWAWFGPLRRASENQLVHAARQQSHFLESVRGVRTIKLFNRPEQRCSAWSALLVEEINAGLHPQKLQLAYRTFNGVLFGLVTIIVIWLGATLVLEGQFSAGMLIAFNSYKEQFNGRVAALIDKIVDVIMLRLHGERLADIVLQDAEPVSGLDGSDDASSENVPSLEVRDVRFRYSEYEPYVLDGVSIHVRAGESVAIVGPSGGGKSTLLNVMLGIMAPSAGSVLLDGQPVGPDHVSRLRRLTGTVLQDDVLFAGSIGDNISFFETSADQQWIEQCARLAAVHDDIAQMPMGYNTLVGDMGTVLSGGQKQRVLLARALYKRPQLLFLDEATSHLDITREAAVNQALETLNITRIIVAHRPETIRSADRVVALANGRIAYDGPVLGEPSAQGPEP

SEQ ID NO: 87 - protein, 712 aa, Pseudomonas syringae
MNLRNTFAFGVGRKLPVVLQAEATECGLACMAMITSYHGQHSDLLSLRQRLSPSMKGVNLKQLTHMAARLGLGSRALRVELNALGQLQLPCVLHWNFNHFVVLKEVNARGVVVHDPGRGVCKLTLDEVSAAFTGVALELWPESDFQPAAAQPPIPLRKLLGRVQGFGRVLTQVLLLALALELCTILSPFFMQTVIDKVLVSADLDLLTVLAIGFGLLLIIQHTVALARAWALMYLGTMLGSQWQINVFTHLLRLPVAFFERRHLGDIVSRFGSLKAIQQTVTTSFIEALLDGLMTVVTLGLMIAYSPKLALVAVVAMIIYGLARWIWFGPLRRASENQLVHAARQQSHFLESARGVRTIKLFNRHEQRCSAWSALLVEEINAGLHPQKLQLAYRAFNGVLFGLVTIIVIWMGARLVLEGQFSAGMLIAFNSYKEQFNGRVAGLIDKIVDVIMLRLHGERLADIVLHDVEPIAGPDTFDDACNDRVPSLEVRQLRFRYSEHEPYVLDDVSIHIRAGESVAIVGPSGGGKSTLLNVMLGILAPCAGSVLLDGLPVGPENIGRLRRITGTVLQDDVLFAGSIGDNISFFETGADQKWIEQCAHLAAVHDDIAQMPMGYNTLVGDMGTVLSGGQRQRVLLARALYKRPQLLFLDEATSHLDINREAAVNQALQTLNITRIIVAHRPETIRTADRVVALAQGRVAYDGPVLNEAPSA

SEQ ID NO: 88 - protein, 712 aa, Pseudomonas syringae
MNLRNTFAFGVGRKLPVVLQAEATECGLACMVMIASYHGQHSDLLSLRQRLSPSMKGVNLKQLTHMAARLGLGSRALRVELNALGQLQLPCVLHWNFNHFVVLKEVNARGVVVHDPGRGLCKLTLDEVSAAFTGVALELWPESDFQPAAAQPPIPLRKLLGRVQGFGRVLTQVLLLALALELCTILSPFFMQTVIDKVLVSADLDLLTVLAIGFGLLLIIQHTVALARAWALMYLGTMLGSQWQINVFTHLLRLPVAFFERRHLGDIVSRFGSLKAIQQTVTTSFIEALLDGLMTVVTLGLMIAYSPKLALVAVVAMIIYGLARWIWFGPLRRASENQLVHAARQQSHFLESARGVRTIKLFNRHEQRCSAWSALLVEEINAGLHPQKLQLAYRAFNGVLFGLVTIIVIWMGARLVLEGQFSAGMLIAFNSYKEQFNGRVAGLIDKIVDVIMLRLHGERLADIVLHDAEPIAGPDTFDDACNDRVPSLEVRQLRFRYSEHEPYVLDDVSIHIRAGESVAIVGPSGGGKSTLLNVMLGILAPCAGSVLLDGLPVGPENIGRLRRITGTVLQDDVLFAGSIGDNISFFETGADQKWIEQCAHLAAVHDDIAQMPMGYNTLVGDMGTVLSGGQKQRVLLARALYKRPQLLFLDEATSHLDINREAAVNQALQTLNITRIIVAHRPETIRTADRVVALAQGRVAYDGPVLNEAPSA

SEQ ID NO: 89 - protein, 716 aa, Pseudomonas syringae
MNLKNAFTFGVGRKLPVVLQAEATECGLACMAMIAGYHGQHSDLFSLRERLSPSMKGVNLKQLTRMAAQLGLGSRALRVELSALGQLQLPCVLHWNFNHFVVLKEVTAQGVVVHDPARGLCKLSLDEVSAAFTGVALEVWPESDFQPAAARPPIPLRKLLGRVQGFGRVLTHVLLLALALELCAILSPFFMQTVIDKVLVSADLDLLAVLAIGFGILLIIQHTVALARGWALMYLGTMLGSQWQINVFTHLLRLPVAFFERRHLGDIVSRFGSLKSIQQTVTTSFIEAILDGLMTVITLGLMIAYSPKLALVAVVAMVIYALARWAWFGPLRRASENQLVHAARQQSHFLESVRGVRTIKLFNRHEQRCSAWSALLVEEINAGLHPQKLQLAYRTFNGVLFGLVTIIVIWLGARLVLEGQFSAGMLIAFNSYKEQFNSRVAGLIDKIVDVIMLRLHGERLADIVLQEAEPVSGLDAFDEASIEKVPSLEVRDLRFRYSEHEPYVLDGVSIHVRAGESVAIVGPSGGGKSTLLNVMLGILAPSAGSVLLDGLPVGPEHVSRLRRLTGTVLQDDVLFAGSIGDNISFFETDADQQWIEQCARLAAVHDDIAQMPMGYNTLVGDMGTVLSGGQKQRVLLARALYKKPQLLFLDEATSHLDIAREAAVNQALQALNITRIIVAHRPETIRSADRVVALANGRIAYDGPVLDEPSARGSEP

SEQ ID NO: 90 - protein, 719 aa, Raoultella planticola
MDLFHKLNLSFRHKLPVLRQTQAAECGLTCVGMIAGYYGYQIDMVSLRHRFPSSQKGSTLADVMSFTQKLDMGCRAVRLELDELNKLKLPCILHWDMNHFVVLKSINKNTLTIHDPARGARKVAMDEVSRSFTGVALELYPAATFERKDEKKSISMLSLIRNVSGIGSAFIQVALLSLALEFFGIITPFYMQWVIDQVLVSADRDLLTLLGVAFIFITLFQNLIGALRSWVTTWFSSMLSVQWSSNLCAHLLGLPLSYFEDRHVGDILSRFGSIANIQSTLTGRFISSIFDGIMALVTLGVIFAYNTSLTFIVIGLFLLYALIRWISFEPFRQASEDQLLASAQAQSQLLESIRGVQAIKLNNKQDLRVSTFTNETVQSTNKGITTQKLSIGFSTLQGTISGVGKIVLIWLAAAQVLDGNFTSGMLVAFISFSDQFISRSAGLINALIDFKMLRLHGERLADIVLSEKEPDLASSASLPDREGPVHLAIHQLSFRYSATDGEIFTGFNLEIEAGESIAIIGPSGQGKTTLAKLLLGLLKPENGSICINGIDHRKLGMTLYRDLIGSVMQTDQLFAGSIIDNISFFDASLDRLRVERVARIAQIHDDIMAMPMGYNSMVGDMGSSLSGGQIQRVILARALYRNPRLLILDEATSHLDIARESAINDAIRHMNVTRIIIAHRPETILSADRIIEIHSRGVIEVAKHDFIHAIKNSQSVLRV

SEQ ID NO: 91 - protein, 719 aa, Raoultella planticola
MDLFHKLNLSFRHKLPVLRQTQAAECGLTCVGMIAGYYGYQIDMVSLRHRFPSSQKGSTLADVMSFTQKLDMGCRAVRLELDELNKLKLPCILHWDMNHFVVLKSINKNTLTIHDPARGARKVAMDEVSRSFTGVALELYPAATFERKDEKKSISMLSLIRNVSGIGSAFIQVALLSLALEFFGIITPFYMQWVIDQVLVSADRDLLTLLGVAFIFITLFQNLIGALRSWVTTWFSSMLSVQWSSNLCAHLLGLPLSYFEDRHVGDILSRFGSIANIQSTLTGRFISSIFDGIMALVTLGVIFAYNTSLTFIVIGLFLLYALIRWISFEPFRQASEDQLLASAQAQSQLLESIRGVQAIKLNNKQDLRVSTFTNETVQSTNKGITTQKLSIGFSTLQGTISGVGKIVLIWLAAAQVLDGNFTSGMLVAFISFSDQFISRSAGLINALIDFKMLRLHGERLADIVLSEKEPDLASSASLPDREGPVHLAIHQLSFRYSATDGEIFTGFNLEIEAGESIAIIGPSGQGKTTLAKLLLGLLKPENGSICINGIDHRKLGMTLYRDLIGSVMQTDQLFAGSIIDNISFFDASLDRLRVERVARIAQIHDDIMAMPMGYNSMVGDMGSSLSGGQIQRVILARALYRNPRLLILDEATSHLDIARESAINDAIRHMNVTRIIIAHRPETILSADRIIEIHSRGVIEVAKHDFIHAIKNSQSVLQV

SEQ ID NO: 92 - protein, 707 aa, Serratia sp.
MVFSWQKTPLILQSEMNECGLACLAMMAGYFGKRIDLASARTLHGASSHGMTLRDLITAFERVGMTARASRVELDELRSLTRPAILHWSFNHFVVLVKVTRRGAVILDPAIGRRSISLRELSDKFTGILMEAWPAETFDKKPLEMNVTVSDLFRGVRGLRRIFTGVLMLSVLVELLSIAVPAASQFTIDTLVRSSDREGIFFVGIVVIAALLIKSAFSVVRAWILMNLRYTLGVKWAEMFFNRLIRLTLSFFEKRHTGDIASRFQSLTAIQEAFTADMVASLLDAIVIVISMAIIFTYSPVLAIGPLIAACAYAALKAGLFSTYRNRKIEHIAFEAVQSSHFLETVRAISAIKMLNLTPVRRREWVNHVVNSTHAGNQLFKLDLLTNTAAVLLVGFSGIYVLSVGAIGFDKGITTGALLAVMLYADMVITRTVKLVNAVSDFCLVSMHSQRLTDVAVSPVERDEGEHVSPQLNGHIVIRNLAFRHSQAERNIFEGINLEVMPGENVAIVGPSGCGKSTFLHVLAGLYESTEGDVFINNVGMSGMGKRDIREHVAFVMQDDKLLAGTIQQNITGFTASPDMERMAECANHASIDEEISAFPQGYDSMIGDIGSTLSGGQRQRISIARALYRQPHVLLLDEATSDLDIDNEKRITRAIGQLPITRIFVAHRPEMIKSADRVFNLHLNAWVKQESRGTATMLIADKVHSS

SEQ ID NO: 93 - protein, 721 aa, Serratia inhibens
MKFLESLNFGWRRRLPVIQQTQSAECGLACIGMIANFYGHSIDMVSLRRRFSTSLKGATLADIMLMANRLDMAGRALRLDLDELAKLRRPCILHWELNHFVVLKSVSKDKITIHDPARGKRDVPMDEVSRCFTGVALELLPSATFKKVSEKESISMLKLIGNVTGIGSAFAQIMILSVALEVFGILSPFYMQWVLDQVLVSADYDLLTLLGTGFIIIMILQNIIAALRSWVTTWFSSLLSVQWTTNVCSHLLGLPMSYFEERHIGDVVSRFGSISTIQSTLTGRFISSILDGVMAIVTLGMLFAYSSSLTWLVLGLFLAYMIIRWVAFRPFRQANEDQIVASARAQSQLLESIRGVQAVKLNNKQETRIAAYANALVEATNKGIAIERLSIGFSTLQGTISGVGRVALVWLAATQVLEGNFTSGMLVAYVSFSDQFMSRASGLVDAVIEFTMLRLHGERLADIVLTDTEADMENRVALPATLSDRSAPAVDIENLCFRYSETEPLVINHCNFSIRSGESVAIIGPSGQGKTTLAKLLLGLLKPESGSITIDGLDIRKMGMHHYRDRIGCVMQDDILFAGSIADNICFFDSVPNQALIEKSATIAQIHSDIMAMPMGYHSLVGDMGSSLSGGQIQRVLLARALYRQPDLLILDEASSHLDIERERFINESIKSMPMTRVIIAHRPETIRSADRVILLSQGVAQEIEKDDFMTVFESEDISNN

SEQ ID NO: 94 - protein, 720 aa, Serratia plymuthica
MDFSHKLNLSFRQKLPLLRQTQAAECGLSCVGMIAGYYGHSIDMVTLRQRFPTSLKGSTLSDVMSIAQNMGMSCRAVRLELSELQKLSLPCVLHWDMNHFVVLKSIAGNQVVIHDPARGIRKVPMEEVSRSFTGIALELYPVASFEIRDEKKSISMLSLIRNVSGIGSAFWQVALLSLVLEFFGIISPFYMQWVIDQVLVSADHELLTLLGVAFIGITLFQNIISALRAWVTTWFSSMLSVQWSSNLCTHLLGLPLSYFEERHVGDILSRFGSISNIQNTLTGSFISSIFDGIMSLVTLMVIFTYNATLSFVVIGLFLSYAVIRWVSFEPFRQANEDHLMAAALAQSQLLESIRGVQAIKLNNKQDLRVSAYANEMVEAANKGITIQKLSIGFNTLQSIISGIGKILLIWLAAVQVLEGNFTSGMLIAFITFSDQFLSRSSGLINALIEFKMLRLHGERIADIVLTEKEKNMESKVTLPEDNGPVRLDVRALSFRYSSTDTAIFSDFNLSIQAGESVAIIGPSGQGKTTLAKLLLGLLKPESGTICINDIDHTKLGMSLYRDLIGSVMQSDMLFAGSIMDNINFFDANLDRRHVERVARIAQIHDDIMAMPMGYNSMVGDMGSSLSGGQIQRVILARALYRNPRILILDEATSHLDVARESAINNAIKHMDMTRILIAHRPETILSADRIIQINRHGAMEIEKSDFIRLVNASQPILHAS

SEQ ID NO: 95 - protein, 714 aa, Stenotrophomonas maltophilia
MADGSASAERRRPDRHSRADVRAELRFGWRRDTPLILQAETAECGLACLAMVAGHHGHHVDMLGMRAKFGSSQKGVTLRQMMHLATALGMEGRPLKLDLGNLSRLKLPCILHWGMNHFVVLCKIGPRSAVIHDPARGVRKLDLLEVSSQFTGVALELIPGADFRPAHRSPSISLRRLAGTIQGLVPACLQVLGLALALEAFILIGPFYLQWTMDQVIVSADRDLLSLLAIGFAIVAVFQVVITALRAWLLSWISATTSAQWMSNMVGHLLRLPLAWFEKRQVGDIVSRLGSVQIIQSTLTVQFIGSLLDGLMSLFVLLIMTFYSPGLAALVAGLFLLYTMLRWVFFAPLWRANEDQIVHAARQQGEILESIRGILPIKLANQQNVRRARYANASVQVINRGIRIQQLNISFGIINGLIFGLGRVALIWIAASLVMDEKFSVGMMVAFVAFADMFIHRSTSLADKWVDFRMLRLHAERLSDIALSEQESPDGVDSCHVPDDSSVEFKNVSFRYSDGEPWVLRNCSFKVESGRSIAITGVSGSGKTTLAKIMLGLLKPSEGEVIFGGQKIDDMGHGRFRGLVGAVMQEDQLFAGTIGDNISFGDQDSTDERIREAATLAAVSDDIEAMPMGYRTLVGDMGSSLSGGQKQRVILARALYRRPRLLVLDEATSHLDLECERRVNAAVKMSSMTRVVIAHRLETIQSADERFEIKHREC

SEQ ID NO: 96 - protein, 738 aa, Xanthomonas sp.
MKDVLTPSADGDAEPDRSAEPSLVFSSKRRVPHIRQTESSECGLACIAMLLSYYGHETSLGELRNRFTVSTSGATLASLIEIADANGLTTRPLRLELYELPKLSLPCMVHLHHGHFVVLTAVRGGHVHISDPAAGVKKIKLDEFDELFSGIALEAHPGPAFKKIKSVPSVSLRDLAGSLRSLVPAFWGVGILALFLEGCALIAPQYLRLTMDQVLSVKDDGLVTTLAIGFSIVLLVQLVLTVARKWTLLWISSTTSLRWSSNLFRHLVSLSQSYFVKRHTGDISSRFQSIYSIQQSLTTKMMEAVIDGVMSFLMLLLLMSYDAPLAFALLAFTIAYVGSRYAYYAKLKEANLDQINIDARRQSLLYETVRSIQPIKLFNKSALWASRFTNYSAKATNATIGAERIRIGFDSVQLLIQGLCRIFVIWEGTRLVLSGDMTVGVLTVFLIYATQFSQRSINLADYLMQLRLLRLHTERISDITHSEPESFLHGNGALEDAAPTIAIANGYFRYSSTDKWIMSALQLRAPAGQVIAIVGNSGVGKTTLIRVLAGLEDLQVGDFLVNGEDLRKVGKSSYRSKVSIVMQGDNLLSGTLSENISMFDEHIDQERLVQAAKLACIHDDIQRMPMGFNTRVGDLGNTLSGGQKQRVFLARAFYRRTKLLLMDEPTTGIDEQMGIQLMKNIKSIGATTVIVTHDKNISRMCDMHYLFVGGALRPLIKEQPSKFERDEAGETAEKSSQQ

SEQ ID NO: 97 - protein, 700 aa, Xanthomonas sp.
MTMADGSPTLLWSQKKRVDPIMQSEAAECALVCLAMLAQFYGNDCGLPALRRRYSSTLKGMSLNQVIEVADDMGFDCRALRAELDYLREVQAPVILHWNMNHFVVLAGTKGSKLVIIDPAQGRRLMPMGEVSKYFTGVLLELEPSASFRRASKSPTVPLSALTGRISGLKRVLIQIFALALAIEALGLSIPLQAQWAVDQISESNGKVILTVAAIFSLVVIIQAGLNLARAWLISWLGANISTQWVINIFSHLMRLPLDFFEKRHLGDIMSRFGSIHWIQAILTGSFVTALLDGLTGSLALVLLFIYSTGMATVAVAVATLYALGRFLMFGALWRASEQSMVFDARQQSELMESVRGIQAIKLANRQLERRARLANATLEAAKRGITAERIKLGFGAASQGIFGLQRVFLLSAGAYLISRNAMTAGMMVAALSYADQFSIRVGSLIDNAVELKMLGLHLNRLADITTADEEPARGRLEGALRSAPEVQVKGLGYRYSEWEPWIFRNLSFTIAPGQSVALVGPSGCGKTTLAKIVLGLIAPQEGEVTVTDQPSPVCRSAIPAEGMAAVMQDDCLFSGTIADNVAFFDSSAELSKIEAAARAAGIHDDIVKMPMGYETLVGDMGSTLSGGQKQRILLARALYTQPKILVLDEATSHLDSKNEKVVNDAVMDLEITRIIIAHREQTIAMADRVFDLSRQTWIR

SEQ ID NO: 98 - protein, 718 aa, Xanthomonas sp.
MSRWQAQLRRASGVRGLPMILQGQVGECGLAAMAMIAHYHGCHIGLAELRRRFLLSRQGTNLANLVAIAQALGLQARALRLEMDALPELQLPCIVHWDLNNFVVLKRVGARRLQIHDPASGPRSLTPGEFARHFSGIALELSPAADFRPQAAAPAVALSSLIGRVHGLGRAVWQVLALAFALEVLSLGMPFQLQWIVDQAVPSADIGLIHVLGAGFLLLVVLQSCIALLRGWLIASVSAQLGFQWMGQVFAHLLALPLAYFEKRHLGGIQSRFASIVQVQRTLTTGFTQTLVDGVLVIGTLGLMLVYSVGLSAITLVAVALYAATRWLWLRRMREAAAEQLLWDARQHTHLLESVRGIQGVRLFGRQQVRRMDWTHLLAEQTNAQLRLAQGEVWQSSLQRLLFGCERVLVIWLAAFAILRAELSLGMLLAFMAYREQFAMRLSELIDRLVEFRLLRVHLERVADIVHQPREEADQRIDAPAWNDTTIELCGIGFRYADDTPAVLEDVSVRIASGECVAITGASGCGKTTLVKVILGLLQPSTGQVKIGGRPLSLAALAPYRAIVGTVMQDDLLFTGSVSENISFFDPEPDQAQIERCARIAGVHQEVEQMPLGYASLLSEAGTGLSGGQRQRVLLARALYRSPRILVLDEATSHLDVMNEQRVNRAIQAMQVTRIIVAHRSETVAMAARVITLEQGRVVSDQPIADWQRLQERSTAAD

SEQ ID NO: 99 - protein, 710 aa, Xanthomonas sp.
MKSIIQSEAAECGLVALAITAASHGLNIGLPEMRRRFPLSLKGAKLNHLIRIAQQLGFATRPLRLDMEHLSKLKLPCVLHWDLNHYVVLAKVGKLKATIFDPALGERTLLMDEVSKHFTGVALELTPTAEFKPKKATSSVSVRQLTGSIRGLGGAVLQILLLSFALQVFVILAPFYMQWVVDQVLVSGDHALLMVVGLGFGLALLLQIGIGLLRGWSVVYLSSTLGLQWMTNVFAHLLKLPLFFFEKRHLGDVTSRLSSVQTIQNTLTTSFVESIIDGLMTIVTLGLMLIYSWKLALVTLLAVGLYLAIRAIAFHPMRNCTELQLVAAAKQQTHLLESLRGIQSLKVAGEESQRRSTYEGLLNDTVNQNARLARMGLGFSTASQLVFGIERIAVIWIGATMTLDNVFSVGMLVAYLAYKDQFAKRVSALIDKWIEFRMLRLHGERLADIVLTPPECDYALPEVLPPTEPRIEVEGLSFRYAEGEPWVLKECSLSIHAGESVAIIGPSGCGKTTLLKLLLGLLQPTEGVIRIGGHDVHKIGPRNVRAIVGAVMQDDQLFAGSIADNISFFDQDFDLERIEAAARLAAVHEDIAAMPMGYHGLIGDMGSSLSGGQKQRIILARALYRQPKLLFLDEATSHLDVMCERLVNEAVKQLKVTKVLVAHRPETIASADRVIVLEQGRVVQEFHPHAALSDVLPARDIERAHGPAVV

SEQ ID NO: 100 - protein, 700 aa, Xanthomonas sp.
MTMADGSPTLLWSQKKRVDPIMQSEAAECALACLAMLAQFYGNHCGLPALRRRYSSTLKGMSLNQVIEVADDMGFDCRALRAELDYLREVQAPVILHWNMNHFVVLAGTKGSKLVIIDPAQGRRLMPMGEVSKYFTGVLLELEPSASFRRASKSPTVPLSALTGRISGLKRVLIQIFALALAIEALGLSIPLQAQWAVDQISESNGKVILTVAAIFSLVIIIQAGLNLARAWLISWLGANISTQWVINIFSHLMRLPLDFFEKRHLGDIMSRFGSIHWIQAILTGSFVTALLDGLTGSLALALLFIYSTGIATIAVVVATLYALGRFLMFGALWRASEQSMVLDARQQSELMESVRGIQAIKLANRQLERRARLANATLEAAKRGVTAERIKLGFGAASQGIFGLQRVFLLSAGAYLISRNAMTAGMMVAALSYADQFSTRVGSLIDNAVELKMLGLHLNRLADITTADEEPARGRLEGALRSAPEVQMKGLGYRYSEWEPWIFRNLSFTIAPGQSVALVGPSGCGKTTLAKIVLGLLAPQEGEVTVTDQPRPVCRSAIPAEGMAAVMQDDCLFSGTIADNVAFFDSSAELSEIEAAARAAGIHDEILKMPMGYETLVGDMGSTLSGGQKQRLLLARALYRQPKILVLDEATSHLDSDNEKVVNDAVMDLEITRIIIAHRKETIAMADRVFDLSTQTWIR

SEQ ID NO: 101 - protein, 700 aa, Xanthomonas citri
MTMADGSPTLLWSQKKRVDPIMQSEAAECALACLAMLAQFYGNHCGLPALRRRYSSTLKGMSLNQVIEVADDMGFDCRALRAELDYLREVQAPVILHWNMNHFVVLAGTKGSKLVIIDPAQGRRLMPMGEVSKYFTGVLLELEPSASFRRASKSPAVPLSALTGRISGLKRVLIQIFALALAIEALGLSIPLQAQWAVDQISESNGKVILTVAAIFSLVIIIQAGLNLARAWLISWLGANISTQWVINIFSHLMRLPLDFFEKRHLGDIMSRFGSIHWIQAILTGSFVTALLDGLTGSLALALLCIYSTGMATVAVVVATLYALGRFLMFGALWRASEQSMVLDARQQSELMESVRGIQAIKLANRQLERRARLANATLEAAKRGVTAERIKLGFGAASQGIFGLQRVFLLSAGAYLISRNAMTAGMMVAALSYADQFSTRVGSLIDNAVELKMLGLHLNRLADITTADEEPARGRLEGALRSAPEVQMKGLGYRYSEWEPWIFRNLSFTIAPGQSVALVGPSGCGKTTLAKIVLGLLAPQEGEVTVTDQPRPVCRSAIPAEGMAAVMQDDCLFSGTIADNVAFFDSSAELSEIEAAARAAGIHDEILKMPMSYETLVGDMGSTLSGGQKQRILLARALYRQPKILVLDEATSHLDSDNEKVVNDAVMDLEITRIIIAHRKETIAMADRVFDLSTQTWIR

SEQ ID NO: 102 - protein, 740 aa, Xanthomonas citri
MKDVLTPSANGDAETDRSAEPSLVFSSKRRVPHIRQTESSECGLACIAMLLSYYGHETSLGELRNRFTVSTSGATLASLIEIADANGLTTRPLRLELYELPKLSLPCMVHLHHGHFVVLTAVRGGHVHISDPATGVKKIKLDEFDELFSGIALEAHPGPAFKKIKSVPSVSLRDLAGSLRSLVPAFWVVGILALFLEGCALIAPQYLRLTMDQVLSVKDDGLVTTLAIGFSIVLLVQLVLTVARKWTLLWISSTTGLRWSSNLFRHLVSLPQSYFVKRHTGDISSRFQSIYSIQQSLTTKMLEAVIDGVMSFLMLLLLMSYDAPLSFALLAFTIAYVGSRYAYYAKLKEANLDQINIDARRQSLLYETVRSIQPIKLFNKSALWASRFTNYSAKATNATIGAERIRIGFDSVQLLIQGLCRIFVIWEGTRLVLSGDMTIGVLTVFLIYATQFSQRSINLADYLMQLRLLRLHTERISDITHSEPESFLHGNGALEDAAPAIAIANGYFRYSSTDKWIMSALQLRAPAGQVIAIVGNSGVGKTTLIRVLAGLEDLQVGDFLVNGEDLRKVGKSSYRSKVSIVMQGDNLLSGTLAENISMFDEHIDQERLSQAAQLACIHDDIQRMPMGFNTRVGDLGNTLSGGQKQRIFLARAFYRRTKLLLMDEPTTGVDERMGIQLMKNIKSIGATTVIVTHDKNISRMCDMHYLFVGGNLKPLIKEKSEQSEANEFAEQLKIQARNEE

SEQ ID NO: 103 - protein, 704 aa, Xanthomonas oryzae
MKAIVQAEASECGLASLAMVASAHGMQLDLPELRRRFHLSLKGIRLNQLIEIAQTLGFSTRPLRLEMEQLDQLSLPCILHWDLNHFVVLAKVGKSKATILDPAIGERRLSLGEVSQHFTGVALELTPTAEFKQHKAAPSISARQLTGPIRGLWSALSQIALLSLALQVFVILAPFYTQWVVDQVLVSADRDLLVVLGLGFGLALLLQVGIGLLRGWSVVSLSSRLGLQWMGNVFAHLLKLPLDFFEKRHLGDVTSRMSSVQTIQHTLTTSFVEAMIDGVMAMVTLVLMLVYSWKLALVTLLAVALYLGIRAIAYRPMRDRTEQQLVAAAKQQTHLLESLRGMQSLKVAGEESVRRSTYENLLNDTVNQDVKLARMSLGFNTASQLVFGLERIAVIWIGARLALDNVFSVGMLVAYLAYKDQFAMRVSGLIDKWIEFRMLRLHGERLADIVLTPPEKQHAQPHALPPAEPSIEVEGLSFRYADGEPWVVKDCSFTIAPGESVAIIGGSGCGKTTLVKLLLGLLTPSEGTIRIGGHDLHKLGPRTVRAMIGVVMQDDQLFAGSIADNIGFFDTDFDLERIKAAAQLAAVHEDIAAMPMGYHSLIGDMGSSLSGGQKQRIILARALYRQPKLLFLDEATSHLDVTRERLVNEAVKHLQLTKVIVAHRPETIASADRILVMEHGRIVQEVKLQQSPEIPHPEDHVLSA

SEQ ID NO: 104 - protein, 704 aa, Xanthomonas oryzae
MKAIVQAEASECGLASLAMVASAHGMQLDLPELRRRFHLSLKGIRLNQLIEIAQTLGFSTRPLRLEMEQLDQLSLPCILHWDLNHFVVLAKVGKSKATILDPAIGERRLSLGEVSQHFTGVALELTPTAEFKQHKAAPSISARQLTGPIRGLWSALSQIALLSLALQVFVILAPFYTQWVVDQVLVSADRDLLVVLGLGFGLALLLQVGIGLLRGWSVVSLSSRLGLQWMGNVFAHLLKLPLDFFEKRHLGDVTSRMSSVQTIQHTLTTSFVEAMIDGVMAMVTLVLMLVYSWKLALVTLLAVALYLGIRAIAYRPMRDRTEQQLVAAAKQQTHLLESLRGMQSLKVAGEESVRRSTYENLLNDTVNQDVKLARMSLGFNTASQLVFGLERIAVIWIGARLALDNVFSVGMLVAYLAYKDQFAMRVSGLIDKWIEFRMLRLHGERLADIVLTPPEKQHAQPHALPPAEPSIEVEGLSFRYADGEPWVVKDCSFTIAPGESVAIIGGSGCGKTTLVKLLLGLLTPSEGTIRIGGHDLHKLGPRNVRAMIGVVMQDDQLFAGSIADNIGFFDTDFDLERIKAAAQLAAVHEDIAAMPMGYHSLIGDMGSSLSGGQKQRIILARALYRQPKLLFLDEATSHLDVTRERLVNEAVKHLQLTKVIVAHRPETIASADRILVMEHGRIVQEVKLQQSPEIPRPEDHVLSA

SEQ ID NO: 105 - protein, 719 aa, Xanthomonas oryzae
MSRWLAQLRRASGERALPMILQGQVGECGLAAMAMIAHYHGCQIGLAELRRRFLLSRQGTNLANLVAIAQALGLQARALRLEMDGVPDLQLPCIVHWDLNHFVVLKRAGARRLQIHDPASGPRVLTPGEFARHFSGIALELSPTADFRAQAAAPPVALSSLIGRVHGLGRAVWQVLALAFALEVLSLGMPFQLQWIVDQAVPSADIGLIHVLGAGFLLVVVLQSCIGLLRGWLIASVSAQLGFQWMGQVFAHLLALPLAYFEKRHLGGIQSRFASITQVQRTLTTGFTQTLVDGVLVVGTLGLMLVYSAGLSAITLVAVALYAATRLLWLSRMREATAEQLLWDARQHTHLLESVRCIQGVRLFGRQQVRRMDWTHLLAEQTNAQLRLAQGEVWQSAIKLLLFGGERVLVIWLAAFAILRAELSLGMLLAFMAYREQFAMRLSALIDRLVEFRLLRVHLERVADIVHQPREDADQRVDAPDWSDTTIEVCGIGFRYADDAPAVLEEVNIRIGSGECVAITGPSGCGKTTLVKVILGLLQPSTGQVKIGGRPLTRAALAHYRGIVGTVMQDDLLFTGSVSENISFFDPEPDQGQIERCARIAGVHQEVEQMPLGYASLLSEAGTGLSGGQRQRVLLARALYRAPRILVLDEATSHLDVINEHRVNHAIQAMQVTRIIVAHRRETVSMAARVITLERGRVTSDQPIAAWQRLQEHSAAAAD

SEQ ID NO: 106 - protein, 719 aa, Xanthomonas oryzae
MSRWLAQLRRASGARALPMILQGQVGECGLAAMAMIAHYHGCQIGLAELRRRFLLSRQGTNLANLVAIAQALGLQARALRLEMDGVPDLQLPCIVHWDLNHFVVLKRVGTRRLQIHDPASGPRGLTPGEFARHFSGIALELSPTADFRAQSAAPPVALSSLIGRVHGLGRAVWQVLALAFALEVLSLGMPFQLQWIVDQAVPSADIGLIHVLGAGFLLLVVLQSCIGLLRGWLIASVSAQLGFQWMGQVFAHLLALPLAYFEKRHLGGIQSRFASITQVQRTLTTGFTQTLVDGVLVVGTLGLMLVYSAGLSAITLVAVALYAATRLLWLSRMREATAEQLLWDGRQHTHLLESVRCIQGVRLFGRQQVRRMDWTHLLAEQTNAQLRLAQGEVWQSAIKLLLFGGERVLVIWLAAFAILRAELSLGMLLAFMAYREQFAMRLSALIDRLVEFRLLRVHLERVADIVHQPREDADQRVDAPDWSDTTIEVCGIGFRYADDAPAVLEEVNIRIGSGECVAITGPSGCGKTTLVKVILGLLQPSTGQVKIGGRPLTRAALAHYRGIVGTVMQDDLLFTGSVSENISFFDPEPDQGQIERCARIAGVHQEVEQMPLGYASLLSEAGTGLSGGQRQRVLLARALYRAPRILVLDEATSHLDVINEHRVNHAIQAMQVTRIIVAHRRETVSMAARVITLERGRVTSDQPIAAWQRLQEHSAAAAD

SEQ ID NO: 107 - protein, 704 aa, Xanthomonas oryzae
MKPILQAEASECGLASLAMVASAHGMQLDLPELRRQFHLSLKGIRLNQLIEIAQTLGFSTRPLRLEMEQLDQLSLPCILHWDLNHFVVLAKVGKSKATILDPAIGERRLSLGEVSQHFTGVALELTPTAEFKQHKAAPSISARQLTGPIRGLWSALSQIALLSLALQVFVILAPFYTQWVVDQVLVSADRDLLVVLGLGFGLALLLQVGIGLLRGWSVVSLSSRLGLQWMGNVFAHLLKLPLDFFEKRHLGDVTSRMSSVQTIQHTLTTSFVEAMIDGVMAMVTLVLMLVYSWKLALVTLLAVALYLGIRAIAYRPMRDRTEQQLVAAAKQQTHLLESLRGMQSLKVAGEESVRRSTYENLLNDTVNQDVKLARMSLGFNTASQLVFGLERIAVIWIGARLALDNVFSVGMLVAYLAYKDQFAMRVSGLIDKWIEFRMLRLHGERLADIVLTPPEKQHAQPHALPPAEPSIEVEGLSFRYADGEPWVVKDCSFTIAPGESVAIIGGSGCGKTTLVKLLLGLLTPSEGTIRIGGHDLHKLGPRNVRAMIGVVMQDDQLFAGSIADNIGFFDTDFDLERIKAAAQLAAVHEDIAAMPMGYHSLIGDMGSSLSGGQKQRIILARALYRQPKLLFLDEATSHLDVTRERLVNEAVKHLQLTKVIVAHRPETIASADRILVMEHGRIVQEVKLQQSPEIPRPEDHVLSA

SEQ ID NO: 108 - protein, 700 aa, Xanthomonas oryzae
MTMADGSPTLLWSQKNRVDPIMQSEAAECALACLAMLAQFYGNDCGLPALRRRYSSTLKGMSLNQVIEVADDMGFDCRALRAELDYLREVQAPVILHWNMNHFVVLAGTKGSKLVIIDPAQGRRLMPMGEVSKYFTGVMLELEPSASFRRASKSPTVPLSALTGRISGLKRVLIQIFALALAIEALGLSIPLQAQWTVDQISESNGKVILTVAAIFSLVIIIQVGLNLARAWLISWLGANISTQWVINIFSHLMRLPLDFFEKRHLGDIMSRFGSIHWIQSILTGSFVTALLDGLTGSLALVLLFIYSTGMATVAVAVATLYALGRFLMFGALWRASEQSMVFDARQQSELMESVRGIQAIKLANRQLERRARLANATLEAAKRGVTAERIKLGFGAASQGIFGLQRVFLLSAGAYLISRNAMTAGMMVAALSYADQFSTRVGSLIDNAVELKMLGLHLNRLADITTADQEPARGRLEGALRSAPEVQVKGLGYRYSEWEPWIFRNLSFTIAPGQSVALVGPSGCGKTTLAKIVLGLLAPQEGEVTVTDQPSPVCRSAIPAKGMAAVMQDDCLFSGTIADNVAFFDSSAELSEIEAAARAAGIYDDIVKMPMGYETLVGDMGSTLSGGQKQRILLARALYTQPKILVLDEATSHLDSKNEKFVNDAVMDLKITRIIIAHREQTIAMADRVFDLSRQTWIR

SEQ ID NO: 109 - protein, 704 aa, Xanthomonas oryzae
MKPILQAEASECGLASLAMVASAHGMQLDLPELRRRFHLSLKGIRLNQLIEIAQTLGCSTRPLRLEMEQLDQLSLPCILHWDLNHFVVLAKVGKSKATILDPVIGERRLSLGEVSQHFTGVALELTPTAEFKQHKAAPSISARQLTGPIRGLWSALSQIALLSLALQVFVILAPFYTQWVVDQVLVSADRDLLVVLGLGFGLALLLQVGIGLLRGWSVVSLSSRLGLQWMGNVFAHLLKLPLDFFEKRHLGDVTSRMSSVQTIQHTLTTSFVEAMIDGVMAMVTLVLMLVYSWKLALVTLLAVALYLGIRAIAYRPMRDRTEQQLVAAAKQQTHLLESLRGMQSLKVAGEESVRRSTYENLLNDTVNQDVKLARMSLGFNTASQLVFGLERIAVIWIGARLALDNVFSVGMLVAYLAYKDQFAMRVSGLIDKWIEFRMLRLHGERLADIVLTPPEKQHAQPHALPPAEPSIEVEGLSFRYADGEPWVVKDCSFTIAPGESVAIIGGSGCGKTTLVKLLLGLLTPSEGTIRIGGHDLHKLGPRNVRAMIGVVMQDDQLFAGSIADNIGFFDTDFDLERIKAAAQLAAVHEDIAAMPMGYHSLIGDMGSSLSGGQKQRIILARALYRQPKLLFLDEATSHLDVTRERLVNEAVKHLQLTKVIVAHRPETIASADRILVMEHGRIVQEVKLQQSPEIPRPEDHVLSA

SEQ ID NO: 110 - protein, 719 aa, Xanthomonas oryzae
MSRWLAQLRRASGERALPMILQGQVGECGLAAMAMIAHYHGCQIGLAELRRRFLLSRQGTNLANLVAIAQALGLQARALRLEMDGVPDLQLPCIVHWDLNHFVVLKRAGARRLQIHDPASGPRVLTPGEFARHFSGIALELSPTADFRAQAAAPPVALSSLIGRVHGLGRAVWQVLALAFALEVLSLGMPFQLQWIVDQAVPSADIGLIHVLGAWFLLVVVLQSCIGLLRGWLIASVSAQLGFQWMGQVFAHLLALPLAYFEKRHLGGIQSRFASITQVQRTLTTGFTQTLVDGVLVVGTLGLMLVYSAGLSAITLVAVALYAATRLLWLSRMREATAEQLLWDARQHTHLLESVRCIQGVRLFGRQQVRRMDWTHLLAEQTNAQLRLAQGEVWQSAIKLLLFGGERVLVIWLAAFAILRAELSLGMLLAFMAYREQFAMRLSALIDRLVEFRLLRVHLERVADIVHQPREDADQRVDAPDWSDTTIEVCGIGFRYADDAPAVLEEVNIRIGSGECVAITGPSGCGKTTLVKVILGLLQPSTGQVKIGGRPLTRAALAHYRGIVGTVMQDDLLFTGSVSENISFFDPEPDQGQIERCARIAGVHQEVEQMPLGYASLLSEAGTGLSGGQRQRVLLARALYRAPRILVLDEATSHLDVINEHRVNHAIQAMQVTRIIVAHRRETVSMAARVITLERGRVTSDQPIAAWQRLQEHSAAAAD

SEQ ID NO: 111 - protein, 719 aa, Xanthomonas oryzae
MSRWLAQLRRASGARALPMILQGQVGECGLAAMAMIAHYHGCQIGLAELRRRFLLSRQGTNLANLVAIAQALGLQARALRLEMDGVPDLQLPCIVHWDLNHFVVLKRVGTRRLQIHDPASGPRGLTPSEFARHFSGIALELSPTADFRAQSAAPPVALSSLIGRVHGLGRAVWQVLALAFALEVLSLGMPFQLQWIVDQAVPSADIGLIHVLGAGFLLLVVLQSCIGLLRGWLIASVSAQLGFQWMGQVFAHLLALPLAYFEKRHLGGIQSRFASITQVQRTLTTGFTQTLVDGVLVVGTLGLMLVYSAGLSAITLVAVALYAATRLLWLSRMREATAEQLLWDARQHTHLLESVRCIQGVRLFGRQQVRRMDWTHLLAEQTNAQLRLAQGELWQSAIKLLLFGGERVLVIWLAAFAILRAELSLGMLLAFMAYREQFAMRLSALIDRLVEFRLLRVHLERVADIVHQPREDADQRVDAPDWSDTTIELCGIGFRYADDTPAVLEEVNIRIGSGECVAITGPSGCGKTTLVKVILGLLQPSTGQVKISGRPLTRAALAHYRGIVGTVMQDDLLFTGSVSENISFFDPEPDQAQIERCARIAGVHQEVEQMPLGYASLLSEAGTGLSGGQRQRVLLARALYRAPRILVLDEATSHLDVINEHRVNHAIQAMQMTRIIVAHRRETVSMAARVITLERGRVASDQPIAAWQRLQEHSAAAAD

SEQ ID NO: 112 - protein, 719 aa, Xanthomonas oryzae
MSRWLAQLRRASGARALPMILQGQVGECGLAAMAMIAHYHGCQIGLAELRRRFLLSRQGTNLANLVAIAQALGLQARALRLEMDGVPDLQLPCIVHWDLNHFVVLKRVGARRLQIHDPASGPRGLTPGEFARHFSGIALELSPTADFRAQSAAPPVALSSLIGRVHGLGRAVWQVLALAFALEVLSLGMPFQLQWIVDQAVPSADIGLIHVLGAGFLLLVVLQSCIGLLRGWLIASVSAQLGFQWMGQVFAHLLALPLAYFEKRHLGGIQSRFASITQVQRTLTTGFTQTLVDGVLVVGTLGLMLVYSAGLSAITLVAVALYAATRLLWLSRMREATAEQLLWDARQHTHLLESVRCIQGVRLFGRQQVRRMDWTHLLAEQTNAQLRLAQGEVWQSAIKLLLFGGERVLVIWLAAFAILRAELSLGMLLAFMAYREQFAMRLSALIDRLVEFRLLRVHLERVADIVHQPREDADQRVDAPDWSDTTIELCGIGFRYADDTPAVLEEVNIRIGSGECVAITGPSGCGKTTLVKVILGLLQPSTGQVKIGGRPLTRAALAHYRGIVGTVMQDDLLFTGSVSENISFFDPEPDQAQIERCARIAGVHQEVEQMPLGYASLLSEAGTGLSGGQRQRVLLARALYRAPRILVLDEATSHLDVINEHRVNHAIQAMQVTRIIVAHRRETVSMAARVITLERGRVASDQPIAAWQRLQEHSAAAAD

SEQ ID NO: 113 - protein, 704 aa, Xanthomonas oryzae
MKAIVQAEASECGLASLAMVASAHGMQLGLPELRRRFHLSLKGIRLNQLIEIAQTLGFSTRPLRLDMEQLDQLALPCILHWDLSHFVVLAKVGKSKATIFDPAIGERRLSLGEVSQHFTGVALELTPTAEFKQHKAAPSISARQLTGPIRGLWSALSQIALLSMALQVFVILAPFYTQWVVDQVLVSADRDLLVVLGLGFGLALLLQVGIGLLRGWSVVSLSSRLGLQWMGNVFAHLLKLPLDFFEKRHLGDVTSRMSSVQTIQHTLTTSFVEAMIDGVMAMVTLVLMLVYSWKLALVTLLAVALYLGIRAIAYRPMRDRTEQQLVAAAKQQTHLLESLRGMQSLKVAGEESVRRSTYENLLNDTVNQDVKLARMSLGFNTASQLVFGLERIAVIWIGARLALDNLFSVGMLVAYLAYKDQFAMRVSGLIDKWIEFRMLRLHGERLADIVLTPPEEHHAQPRALPPTEPSIEVEGLSFRYADGEPWVVKDCGFTIAPGESVAIIGGSGCGKTTLVKLLLGLLVPSEGTIRIGGHDLHKLGPRTVRAMIGVVMQDDQLFAGSIADNIGFFDTDFELERIEAAAQLAAVHEDIAAMPMGYHTLIGDMGSSLSGGQRQRIILARALYRQPKLLFLDEATSHLDVTRERLVNEAVRHLQLTKVIVAHRPETIASADRILVMAHGRIVQEVKSQQSPEISCPEDHVLSA

SEQ ID NO: 114 - protein, 704 aa, Xanthomonas oryzae
MKPILQAEASECGLASLAMVASAHGMQLDLPELRRRFHLSLKGIRLNQLIEIAQTLGCSTRPLRLEMEQLDQLSLPCILHWDLNHFVVLAKVGKSKATILDPAIGERRLSLGEVSQHFTGVALELTPTAEFKQHKAAPSISARQLTGPIRGLWSALSQIALLSLALQVFVILAPFYTQWVVDQVLVSADRDLLVVLGLGFGLALLLQVGIGLLRGWSVVSLSSRLGLQWMGNVFAHLLKLPLDFFEKRHLGDVTSRMSSVQTIQHTLTTSFVEAMIDGVMAMVTLVLMLVYSWKLALVTLLAVALYLGIRAIAYRPMRDRTEQQLVAAAKQQTHLLESLRGMQSLKVAGEESVRRSTYENLLNDTVNQDVKLARMSLGFNTASQLVFGLERIAVIWIGARLALDNVFSVGMLVAYLAYKDQFAMRVSGLIDKWIEFRMLRLHGERLADIVLTPPEKQHAQPHALPPAEPSIEVEGLSFRYADGEPWVVKDCSFTIAPGESVAIIGGSGCGKTTLVKLLLGLLTPSEGTIRIGGHDLHKLGPRNVRAMIGVVMQDDQLFAGSIADNIGFFDTDFDLERIKAAAQLAAVHEDIAAMPMGYHSLIGDMGSSLSGGQKQRIILARALYRQPKLLFLDEATSHLDVTRERLVNEAVKHLQLTKVIVAHRPETIASADRILVMEHGRIVQEVKLQQSPEIPRPEDHVLSA

SEQ ID NO: 115 - protein, 719 aa, Xanthomonas oryzae
MSRWLAQLRRASGARALPMILQGQVGECGLAAMAMIAHYHGCQIGLAELRRRFLLSRQGTNLANLVAIAQALGLQARALRLEMDGVPDLQLPCIVHWDLNHFVVLKRAGARRLQIHDPASGPRVLTPGEFARHFSGIALELSPTADFRAQAAAPPVALSSLIGRVHGLGRAVWQVLALAFALEVLSLGMPFQLQWIVDQAVPSADIGLIHVLGAGFLLVVVLQSCIGLLRGWLIASVSAQLGFQWMGQVFAHLLALPLAYFEKRHLGGIQSRFASITQVQRTLTTGFTQTLVDGVLVVGTLGLMLVYSAGLSAITLVAVALYAATRLLWLSRMREATAEQLLWDARQHTHLLESVRCIQGVRLFGRQQVRRMDWTHLLAEQTNAQLRLAQGEVWQSAIKLLLFGGERVLVIWLAAFAILRAELSLGMLLAFMAYREQFAMRLSALIDRLVEFRLLRVHLERVADIVHQPREDADQRVDAPDWSDTTIEVCGIGFRYADDAPAVLEEVNIRIGSGECVAITGPSGCGKTTLVKVILGLLQPSTGQVKIGGRPLTRAALAHYRGIVGTVMQDDLLFTGSVSENISFFDPEPDQGQIERCARIAGVHQEVEQMPLGYASLLSEAGTGLSGGQRQRVLLARALYRAPRILVLDEATSHLDVINEHRVNHAIQAMQVTRIIVAHRRETVSMAARVITLERGRVTSDQPIAAWQRLQEHSAAAAD

SEQ ID NO: 116 - protein, 704 aa, Xanthomonas vasicola
MKAIVQAEASECGLASLAMVASAHGMQLGLPELRRRFHLSLKGIRLNQLIEIAQTLGFSTRPLRLEMEQLDQLALPCILHWDLNHFVVLAKVGKTKATILDPAIGDRRLSLSEVSQHFTGVALELTPTTEFKQHKAAPSISARQLTGPIRGLWSALSQITLLSLALQVFVILAPFYTQWVVDQVLVSADRDLLVVLGLGFGLALLLQVGIGLLRGWSVVSLSSRLGLQWMGNVFAHLLKLPLDFFEKRHLGDVTSRMSSVQTIQHTLTTSFVEAMIDGVMAMVTLVLMLVYSWKLALVTLLAVALYLGIRAIAYRPMRDRTEQQLVAAAKQQTHLLESLRGMQSLKVAGKESVRRSTYENLLNDTVNQDVKLARMSLGFNTASQLVFGLERIAVIWIGARLALDNVFSVGMLVAYLAYKDQFAMRVSGLIDKWIEFRMLRLHGERLADIVLTPPEEHHAQPHALPPAEPSIAVEGLSFRYADGEPWVVKDCCFTIAPGESVAIIGGSGCGKTTLVKLLLGLLTPSEGTIRIGGHDLHKLGPRNVRAMIGVVMQDDQLFAGSIADNIGFFDTDFDLERIEAAAQLAAVHEDIAAMPMGYHSLIGDMGSSLSGGQKQRIILARALYRQPKLLFLDEATSHLDVTRERLVNEAVKHLQLTKVIVAHRPETIASADRILVMEHGRIVQEVKLQQSPEIPHPEDHVLSA

SEQ ID NO: 117 - protein, 718 aa, Xanthomonas vasicola
MSRWWAQLRRASGLRALPMILQGQVGECGLAAMAMIAHHHGCHIGLAELRRRFLLSRQGTNLANLVAIAQALGLQPRALRLEMDALPDLQLPCIVHWDLNHFVVLKRVGANRLQIHDPASGPRSLTPGEFARHFSGIALELSPTADFRPQSAAPAVALSSLIGRVHGLGRAVWQVLALAFALEVLSLGMPFQLQWIVDQAVPSADIGLIHVLGAGFLLLVVLQACIGLLRGWLIASVSAQLGFQWMGQVFAHLLSLPLAYFEKRHLGGIQSRFASITQVQRTLTTGFTQTLVDGVLVVGTLGLMLVYSVGLSAITLVAVALYAATRLLWLGRMREAAAEQLLWDARQHTHLLESVRGIQGVRLFGRQQVRRMDWTHLLAEQTNAQLRLAQGEVWQSSIKLLLFGCERVLVIWLAAFAILRAQLSLGMLLAFMAYREQFATRLSELIDRLVEFRLLRVHLERVADIVHQPREEAGQRIDAPAWNDTTIELCGIGFRYADDTPAVLEDVSVRIASGECVAITGPSGCGKTTLVKLILGLLKPSAGQVKIGGRPLADAALAHYRAIVGTVMQDDLLFTGSVSENISFFDPEPDQAQIERCARIAGVHQEVEQMPLGYASLLSEAGTGLSGGQRQRVLLARALYRSPRILVLDEATSHLDVMNEHRVNRAIQAMQVTRIIVAHRRETVGMAARVITLERGRVVSDQPIAAWQRLQERSAAAD

SEQ ID NO: 118 - protein, 718 aa, Xanthomonas vasicola
MSRWWAQLRRASGVRALPMILQGQVGECGLAAMAMIAHHHGCHIGLAELRRRFLLSRQGTNLANLVAIAQALGLQPRALRLEMDALPDLQLPCIVHWDLNHFVVLKRVGAHRLQIHDPASGPRSLTPGEFARHFSGIALELSPTADFRPQSAAPAVALSSLIGRVHGLGRAVWQVLALAFALEVLSLGMPFQLQWIIDQAVPSADIGLIHVLGAGFLLLVVLQACIGLLRGWLIASVSAQLGFQWMGQVFAHLLSLPLAYFEKRHLGGIQSRFASITQVQRTLTTGFTQTLVDGVLVVGTLGLMLVYSVGLSAITLVAVALYAATRLLWLGRMREAAAEQLLWDARQHTHLLESVRGIQGVRLFGRQQVRRMDWTHLLAEQTNAQLRLAQGEVWQSSIKLLLFGCERVLVIWLAAFAILRAQLSLGMLLAFMAYREQFATRLSELIDRLVEFRLLRVHLERVADIVHQPREEAGQRIDAPAWNDTTIELCGIGFRYADDTPAVLEDVSVRIASGECVAITGPSGCGKTTLVKLILGLLKPSAGQVKIGGRPLADAALAHYRAIVGTVMQDDLLFTGSVSENISFFDPEPDQAQIERCARIAGVHQEVEQMPLGYASLLSEAGTGLSGGQRQRVLLARALYRSPRILVLDEATSHLDVMNEHRVNRAIQAMQVTRIIVAHRRETVGMAARVITLERGRVVSDQPIAAWQRLQERSAAAD

SEQ ID NO: 119 - protein, 704 aa, Xanthomonas vasicola
MKAIVQAEASECGLASLAMVASAHGMQLGLPELRRRFHLSLKGIRLNQLVQIAQTLGFSTRPLRLEMEHLDQLALPCVLHWDLNHFVVLAKVGKTKATILDPAIGERRLSLSEVSQHFTGVALELTPTTEFKQHKAAPSISARQLTGPIRGLWSALSQIALLSLALQVFVILAPFYTQWVVDQVLVSADRDLLVVLGLGFGLALLLQVCIGLLRGWSVVSLSSRLGLQWMGNVFAHLLKLPLDFFEKRHLGDVTSRMSSVQTIQHTLTTSFVEAMIDGVMALVTLVLMLIYSWKLALVTLLAVALYLVIRAIAYRPMRDRTEQQLVAAAKQQTHLLESLRGMQSLKVAGEESVRRSTYENLLNDTVNQDVKLARMSLGFNTASQLVFGLERIAVIWIGARLALDNVFSVGMLVAYLAYKDQFAMRVSGLIDKWIEFRMLRLHGERLADIVLTPPEEHHAQPHALPPAEPSIAVEGLSFRYADGEPWVVKDCCFTIAPGESVAIIGGSGCGKTTLVKLLLGLLTPSEGTIRIGGHDLHKLGPRNVRAMIGVVMQDDQLFAGSIADNIGFFDTDFDLERIEAAAQLAAVHEDIAAMPMGYHSLIGDMGSSLSGGQKQRIILARALYRQPKLLFLDEATSHLDVTRERLVNEAVKHLQLTKVIVAHRPETIASADRILVMEHGRIVQEVKLQQSPEIPHPEDHVLSA

SEQ ID NO: 120 - protein, 704 aa, Xanthomonas vasicola
MKAIVQAEASECGLASLAMVASAHGMQLGLPELRRRFHLSLKGIRLNQLIEIAQTLGFSTRPLRLEMEQLDQLALPCFLHWDLNHFVVLAKVGKTKATILDPAIGERRLSLSEVSQHFTGVALELTPTTEFKQHKAAPSISARQLTGPIRGLWSALSQIALLSMALQVFVILAPFYTQWVVDQVLVSADRDLLVVLGLGFGLALLLQVGIGLLRGWSVVSLSSRLGLQWMGNVFAHLLKLPLDFFEKRHLGDVTSRMSSVQAIQHTLTTSFVEAIIDGVMTLVTLVLMLVYSWKLALVTLLAVALYLGIRAIAYRPMRDRTEQQLVAAAKQQTHLLESLRGMQSLKVAGEESVRRSTYENLLNDTVNQDVKLARMSLGFNTASQLVFGLERIAVIWIGARLALDNVFSVGMLVAYLAYKDQFAMRVSGLIDKWIEFRMLRLHGERLADIVLTPPEEHHAQPHALPPAEPSIAVEGLSFRYADGEPWVVKDCCFTIAPGESVAIIGGSGCGKTTLVKLLLGLLTPSEGTIRIGGHDLHKLGPRNVRAMIGVVMQDDQLFAGSIADNIGFFDTDFDLERIEAAAQLAAVHEDIAAMPMGYHSLIGDMGSSLSGGQKQRIILARALYRQPKLLFLDEATSHLDLTRERLVNEAVKHLQLTKVIVAHRPETIASADRILVMEHGRIVQEVKLQQSPEIPHPEDHVLSA

SEQ ID NO: 121 - protein, 707 aa, Xylella fastidiosa
MRSVLQSEVSECGLASLAMVANAHGKHLTLSQLRQRFPSSLKGAKLTHLVHIAQQLGFSTRPLRLDMEDLGKLKLPCILHWDLNHFVVLAKVGTTKVTIYDPAIGKRKLSLNEVSKYFTGIALELTTTTEFKQQKPPPSISARQLTGPIRGLWRALAQILLLSLALQVFVVLSPFYMQWVVDQVLVSADRDLLVVLGLGFGLVLLLQIAIGLFRSWSVIYLSSRLGLQWMGNVFAHLLKLPLEYFEKRHLGDLTSRMSSVQSIQKTLTTSFVEAIIDGLMTLVTLGLMLVYSWKLALVTLVAVVLYMVIRAVSYWPVRDCTEQQLVVGAKQQTHLLESLRGIQSLKVACEESLRRSTYENLLNDTVNQEVRLARMSLGFSTASQLVFGLERIAVIWYGATLALSNVFSVGMLIAYLSYKDQFASRISSLIDKWVEFRMLRLHGERLADIVLTPPEEDYELPEMQLPKKEPDIEVEGLSFRYGEDEPWVLKDCSFKVAAGESVAIIGASGCGKTTLMKLLLGLLKPTAGTIRISGRDLHKFGTRNVRSIVGAVMQDDQLFAGSVADNISFFDPDFDLERIEAAAKLAAIHEDIAAMPMGYHALIGDMGSALSGGQKQRVILARALYRQPKLLFLDEATSHLDVMRERLVNDAVHQLKLTKVIVAHRPETIASTDRVVILDHGRIVQTVAMPEDRDDSIQIVHNTIQST

SEQ ID NO: 122 - protein, 707 aa, Xylella fastidiosa
MRSVLQSEVSECGLASLAMVANAHGKHLTLSQLRQRFPSSLKGAKLTHLVHIAQQLGFSTRPLRLDMEDLGKLKLPCILHWDLNHFVVLAKVGTTKVTIYDPAIGKRKLSLNEVSKYFTGIALELTTTTEFKQQKPPPSISARQLTGPIRGLWRALAQILLLSLALQVFVVLSPFYMQWVVDQVLVSADRDLLVVLGLGFGLVLLLQIAIGLFRSWSVIYLSSRLGLQWMGNVFAHLLKLPLEYFEKRHLGDLTSRMSSVQSIQKTLTTSFVEAIIDGLMTLVTLGLMLVYSWKLALVTLVAVVLYMVIRAVSYWPVRDCTEQQLVVGAKQQTHLLESLRGIQSLKVACEESLRRSTYENLLNDTVNQEVRLARMSLGFSTASQLVFGLERIAVIWYGATLALSNVFSVGMLIAYLSYKDQFASRISSLIDKWVEFRMLRLHGERLADIVLTPPEEDYELPEMQLQKKEPDIEVEGLSFRYGEDEPWVLKDCSFKVAAGESVAIIGASGCGKTTLMKLLLGLLKPTAGTIRISGRDLHKFGTRNVRSIVGAVMQDDQLFAGSVADNISFFDPDFDLERIEAAAKLAAIHEDIAAMPMGYHALIGDMGSALSGGQKQRVILARALYRQPKLLFLDEATSHLDVMRERLVNDAVHQLKLTKVIVAHRPETIASTDRVVILDHGRIVQTVAMPEDRDDSIEIVHNTIQST

SEQ ID NO: 123 - protein, 707 aa, Xylella fastidiosa
MRPVLQSEVSECGLASLAMVANAHGKHLTLSQLRQRFPSSLKGAKLTHLVHIAQQLGFSTRPLRLDMEDLGKLKLPCILHWDLNHFVVLAKVGTTKVTVYDPAIGKRKLSLNEVSKYFTGIALELTTTTEFKQQKPPPSISARQLTGPIRGLWRALAQILLLSLALQVFVVLSPFYMQWVVDQVLVSADRDLLVVLGLGFGLVLLLQIAIGLFRSWSVIYLSSRLGLQWMGNVFAHLLKLPLEYFEKRHLGDLTSRMSSVQSIQKTLTTSFVEAIIDGLMTLVTLGLMLVYSWKLALVTLVAVALYMVIRAVSYWPVRDCTEQQLVVGAKQQTHLLESLRGIQSLKVACEESLRRSTYENLLNDTVNQEVRLARMSLGFSTASQLVFGLERIAVIWYGATLALSNVFSVGMLIAYLSYKDQFASRISSLIDKWVEFRMLRLHGERLADIVLTPPEEDYELPEMQLPKKEPDIEVEGLSFRYGEDEPWVLKDCSFKVAAGESVAIIGASGCGKTTLMKLLLGLLKPTAGTIRISGRDLHKFGTRNVRSIVGAVMQDDQLFAGSVADNISFFDPDFDLERIEAAAKLAAIHEDIAAMPMGYHALIGDMGSALSGGQKQRVILARALYRQPKLLFLDEATSHLDVMRERLVNDAVHQLKLTKVIVAHRPETIASTDRVVILDHGRIVQTVAMPEDRDDSIEIVHNTIQST

SEQ ID NO: 124 - protein, 698 aa, Escherichia coli
MTNGSFRQIINQLDMRWRRRVPVIHQTETAECGLACLAMICGHFGKNIDLISLRRKFNLSARGANLAGINGIAEQLGMVTRALSLELDELGALKMPCILHWDFSHFVVLVSVKRNRYVLHDPARGRRYLGREEMSRYFTGIALEVWPGSEFLAETQQIRISLRSLINSIYGIKRTLAKIFCLSVVIEAINLVMPVGTQLVMDHAIPAGDRGLLTLISAGLMFFILLRAAVSMLRAWSSLVMSTLINIQWQSGLFNHLLRLPLAFFERRKLGDIQSRFGSLDTLRATFTTCVVGAIMDSIMVVGVFVMMLLYGGYLTWIVLGFTMVYVLIRLVTYGYYRQISEETLVRGARASSYFMESLYGIATVKIQGMAGIRGTHWLNLKIDAINSGIKLTKMDLLFGGINTFVAACDQVAILWLGASLVIDNQMTIGMFVAFGSFRGQFSDRVASLTSFLLQLRIMSLHNERIADIALHEKEEKKPEIEIVADMSPVSLETTDLSYRYDSQSAQVFSGLNLSVAPGESVAITGASGAGKTTLMKVLCGLFEPDSGKVLVNGTDIRQLGINNYHRMIACVMQDDRLFSGSIRENICGFAEETDDEWMTECARASHIHDVIMKMPMGYETLIGELGEGLSGGQKQRIFIARALYRKPGILFMDEATSSLDTESERFVNAAIKKMNITRVIIAHRETTLRTVDRIISI

SEQ ID NO: 125 - protein, 706 aa, Escherichia coli
MQQDRSMTNGNFRQIINQLDMRWRRRVPVIHQTETAECGLACLAMICGHFGKNIDLISLRRKFNLSARGTNLAGINGIAEQLGMVTRALSLELDELGALKMPCILHWDFSHFVVLVSVKRNRYVLHDPARGRRYLGREEMSRYFTGIALEVWPGSEFLAETQQTRISLRSLINSIYGIKRTLAKIFCLSVVIEAINLLMPVGTQLVMDHAIPAGDRGLLTLISAGLMFFILLRAAVSMLRAWSSLVMSTLINIQWQSGLFNHLLRLPLAFFERRKLGDIQSRFGSLDTLRATFTTCVVGAIMDSIMVVGVFVMMLLYGGYLTWIVLGFTMVYVLIRLVTYGYYRQISEETLVRGARASSYFMESLYGIATVKIQGMAGIRGTHWLNLKIDAINSGIKLTRMDLLFGGINTFVAACDQVAILWLGASLVIDNQMTIGMFVAFGSFRGQFSDRVASLTSFLLQLRIMSLHNERIADIALHEKEEKKPEIEIVADMSPVSLETTDLSYRYDSQSAQVFSGLNLSVAPGESVAITGASGAGKTTLMKVLCGLFEPDSGKVLVNGTDIRQLGINNYHRMIACVMQDDRLFSGSIRENICGFAEETDDEWMTECAKASHIHDVIMKMPMGYETLIGELGEGLSGGQKQRIFIARALYRKPGILFMDEATSSLDTESERFVNAAIKKMNITRVIIAHRETTLRTVDRIFRITN

SEQ ID NO: 126 - protein, 707 aa, Klebsiella sp.
MTNELFEKIITKVNFSLLKKTPVILQSEAAECGIACLAMVCGHYGLDIDLFNFRQRFGSPSQGVTLMSLSKTAEHAGLKSRALSLDLDEIRQLKLPCVLHWGMNHYVVLTKVRKSSFVVHDPALGKRIIGNQEMSNYFTGIALELWPDQNFQQEKAKSRLRLLDLMRNIVGLKSALLKIFAFSVVVEAIGLLLPIGTQLVTDHVIMAHDQSLLSVICIGLVFFTLFRTFISMLRAWTSLTLNTLTNIQWKTTLFDHLTSLPLSFFEKRHLGDIQSRFSSLDTIRSTFTNSIVSGIIDSIMTIGLLIMLTLYGGWLTWVVVGFTLCYAIMRLATYHFYRRVAEEQVIKGARSSSHFMESLYGISTIKALNLKERRSQHWLNINIEACNAGIKQTRFDMMFGGVNTFITAIDQVAVLWLGAIMVIDNNMTLGMFMAFNSYRGQFSQRASSLVDLCMQLRMLSLHNERLSEIVFSEPEKELPAREVFSPDSGAKLEVKNLCYQYDPFSQPIFSNLNITVEPGESVALIGPSGVGKTTLLKVMCGLLSPTSGDVLADNLDITKIGLNNYRQGTACVLQEDRLFSGSLIDNISGFQDNADLDFVMECAKRCNIHDEIMKMPMGYETIVGELGLGISGGQKQRILIARALYRKPSILFMDEATSHLDLRNESVINQSISALSITRIIVAHRPSTIASADRIIDLSQSKTLVPA

SEQ ID NO: 127 - protein, 707 aa, Klebsiella sp.
MTNELFKTVVAKLNFSLINKTPVILQSESSECGIACLAMVCGYYGLNIDLFNFRQRFGSPSQGSSLMSLSKTAEHAGLKSRALALDLDEIKQLKLPCVIHWGMNHYVVLTKVRKNGFIIHDPSLGKRIIGISEMSNNFTGVALELWPDHNFQQEEAKSRLRLLDLMHKIVGLKSALIKIFAFSVVVEAIGLLLPIGTQLVTDHVIMAHDESLLSVICIGLIFFTLFRTFISMLRAWTSLTLNTLTNIQWKTTLFDHLASLPLSFFEKRHLGDIQSRFSSLDTIRSIFTNSIVTGIIDSIMTIGLLVMLTLYGGWLVWVVVGFTVCYAIMRFATYGFYRRVTEEQVVKGARANSHFMESLYGIATIKALNLKGRRSQHWLNLNVDASNAGIKQTRFDMMYGGINTFITSIDQVAVLWLGAIMVIDNEMTLGMFMAFNAYRGQFSQRASNLIDLVMQLRMLSLHNERLSEIAFSEPEKELPSRRVFTENTGVQLEVRNLSYQYDPFSQPIFTNLNITIAPGESVALIGPSGVGKTTLLKVMCGLLSPDSGEILADNLEIKKIGLNNYRLGIACVLQEDRLFSGSLIDNISGFDDNVDLDFVMECAKRCNIHDEIMKMAMGYETIVGELGLGISGGQKQRILIARALYRKPSILFMDEATSHLDLRNESVINQSISSLSITRIIVAHRPSTIASADRVIDLSQSKSMTTA

SEQ ID NO: 128 - protein, 705 aa, Klebsiella sp.
MINELFDKITSRVNFSLRRKAPIILQSEGSECGIACLAMICGYHGLEIDLFNFRHRFGSATQGATLLSLSKTAEQAQLKSRALSLDLDEIQQLKLPCILHWDLNHYVVLIKRKKNSFIIHDPAMGKRVIGIKEMSNHFTGVALELWPDNNFQQEKAKSRLRLIDLMKNISGLKNALLKIFAYSVVVEAINLLLPIGTQLVTDHVIIAHDESLLAVICIGLVMFTLFRTFISMLRAWTSLTLNTLTSIQWKTTLFDHLMSLPLSFFEKRHLGDIQSRFSSLDTIRSTFTNSIISGLIDLIMTVGLIIMLTLYGGWLVWVVIGFTLCYAIMRLATYQFYRRVAEEQVVKGARSSSHFMESLYGISTIKALDLKGRRSEHWLNINIEACNAGIKQTRFDMLFGGINTLITSIDQVVILWLGAVMVIDNEMTLGMFMAFNAFRGQFSQRASSLVDLSMQLRMLSLHNERLSEIVFTEPEKELPARRIFEENRGVALNVQNLSYQYDPFTKPIFSDLNINVLPGESIALVGPSGIGKTTLLKVMCGLLTPTHGEVVVEGLDISKIGLNNYRMSTACVLQEDRLFSGSIADNICSFEDNPDMEFVMHCAKLSNIHDEIMKMPMGYETLVGELGLGISGGQKQRLLIARALYRKPNILFMDEATSHLDLANESYINQSISSLSITRIIVAHRPSTIASADRIIDLSSKNIHK

SEQ ID NO: 129 - protein, 707 aa, Klebsiella sp.
MTNELFKTIVAKLNFSLVNKTPVILQSESSECGVACLAMVCGYYGLNIDLFNFRQRFGSPSQGSSLLSLSKTAEQAGLKNRALALDLDEIKQLKLPCIIHWGMNHYVVLTKVKKNGFVIHDPALGKRIIGISEMSNNFTGVALELWPDHNFQQEEAKSRLRLLDLMHKIVGLKSALIKIFAFSVVVEAIGLLLPIGTQLVTDHVIMAHDESLLSVICIGLIFFTLFRTFISMLRAWTSLTLNTLTNIQWKTTLFDHLASLPLSFFEKRHLGDIQSRFSSLDTIRSIFTNSIVTGIIDSIMTIGLLVMLTLYGGWLVWVVIGFTACYAIMRFATYSFYRRVTEEQVVKGARANSHFMESLYGISTIKALNLKERRSQHWLNLNVDASNAGIKQTRFDMMFGGINTFITSIDQVVVLWLGAIMVIDNEMTLGMFMAFNAYRGQFSQRASNLIDLVMQLRMLSLHNERLSEIAFSEPEKELPSRRVFTANTGVKLEVRNLSYQYDPFSQPIFTNLNITVAPGESVALIGPSGVGKTTLLKVMCGLLSPDSGEILADNLEIQKIGLNNYRLGIACVLQEDRLFSGSLIDNISGFDDNPDLEFVMECAKHCNIHDEIMKMAMGYETIVGELGLGISGGQKQRILIARALYRKPSILFMDEATSHLDLRNESVINQSISALSITRIIVAHRPSTIASADRVIDLSQSKSMATA

SEQ ID NO: 130 - protein, 707 aa, Klebsiella pneumoniae
MTNELFKTVVAKLNFSLVNKTPVILQSESSECGIACLAMVCGYYGLNIDLFNFRQRFGSPSQGSSLMSLSKTAEHAGLKSRALALDLDEIKQLKLPCIIHWGMNHYVVLTKVRKNGFIIHDPALGKRVIGISEMSNNFTGVALELWPDHNFQQEEAKSRLRLLDLMHKIVGLKPALIKIFAYSVVVEAIGLLLPIGTQLVTDHVIMAHDQSLLSVICIGLIFFTLFRMFISMLRAWTSLTLNTLTNIQWKTTLFDHLASLPLSFFEKRHLGDIQSRFSSLDTIRSIFTNSIVTGIIDSIMTIGLLVMLTLYGGWLVWVVVGFTVCYAIMRFATYGFYRRVTEEQVVKGARANSHFMESLYGISTIKALNLKERRSQHWLNLNVDASNAGIKQTRFDMMYGGINTFITSIDQVAVLWLGAIMVIDNEMTLGMFMAFNAYRGQFSQRASNLIDLVMQLRMLSLHNERLSEIAFSEPEKELPSRRVFTENTGVKLEVRNLSYQYDPFSQPIFTNLNITVDPGESVALIGPSGVGKTTLLKVMCGLLSPDSGEILADNLEIKKIGLNNYRFGIACVLQEDRLFSGSLIDNISGFDDNADLDFVMECARRCNIHDEIMKMSMGYETIVGELGLGISGGQKQRILIARALYRKPSILFMDEATSHLDLRNESVINQSISALSITRIIVAHRPSTIASADRVIDLAQSKPVTTA

SEQ ID NO: 131 - protein, 707 aa, Klebsiella pneumoniae
MTNELFKTVVAKLNFSLVNKTPVILQSESSECGIACLAMVCGYYGLNIDLFNFRQRFGSPSQGSSLMSLSKTAEHAGLKSRALALDLDEIKQLKLPCIIHWGMNHYVVLTKVRKNGFIIHDPALGKRVIGISEMSNNFTGVALELWPDHNFQQEEAKSRLRLLDLMHKIVGLKPALIKIFAYSVVVEAIGLLLPIGTQLVTDHVIMAHDQSLLSVICIGLIFFTLFRMFISMLRAWTSLTLNTLTNIQWKTTLFDHLASLPLSFFEKRHLGDIQSRFSSLDTIRSIFTNSIVTGIIDSIMTIGLLVMLTLYGGWLVWVVVGFTVCYAIMRFATYGFYRRVTEEQVVKGARANSHFMESLYGISTIKALNLKERRSQHWLNLNVDASNAGIKQTRFDMMYGGINTFITSIDQVAVLWLGAIMVIDNEMTLGMFMAFNAYRGQFSQRASNLIDLVMQLRMLSLHNERLSEIAFSEPEKELPSRRVFTENTGVKLEVRNLSYQYDPFSQPIFTNLNITVDPGESVALIGPSGVGKTTLLKVMCGLLSPDSGEILADNLEIKKIGLNNYRLGIACVLQEDRLFSGSLIDNISGFDDNADLDFVMECARRCNIHDEIMKMSMGYETIVGELGLGISGGQKQRILIARALYRKPSILFMDEATSHLDLRNESVINQSISALSITRIIVAHRPSTIASADRVIDLAQSKPVTTA

SEQ ID NO: 132 - protein, 707 aa, Klebsiella pneumoniae
MTNELFKTVVAKLNFSLVNKTPVILQSESSECGIACLAMVCGYYGLNIDLFNFRQRFGSPSQGSSLMSLSKTAEHAGLKSRALALDLDEIKQLKLPCIIHWGMNHYVVLTKVRKNGFIIHDPALGKRVIGISEMSNNFTGVALELWPDHNFQQEEAKSRLRLLDLMHKIVGLKPALIKIFAYSVVVEAIGLLLPIGTQLVTDHVIMAHDQSLLSVICIGLIFFTLFRMFISMLRAWTSLTLNTLTNIQWKTTLFDHLASLPLSFFEKRHLGDIQSRFSSLDTIRSIFTNSIVTGIIDSIMTIGLLVMLTLYGGWLVWVVVGFTVCYAIMRFATYGFYRRVTEEQVVKGARANSHFMESLYGISTIKALNLKERRSQHWLNLNVDASNAGIKQTRFDMMYGGINTFITSIDQVAVLWLGAIMVIDNEMTLGMFMAFNAYRGQFSQRASNLIDLIMQLRMLSLHNERLSEIAFSEPEKELPSRRVFTENTGVKLEVRNLSYQYDPFSQPIFTNLNITVDPGESVALIGPSGVGKTTLLKVMCGLLSPDSGEILADNLEIKKIGLNNYRLGIACVLQEDRLFSGSLIDNISGFDDNADLDFVMECARRCNIHDEIMKMSMGYETIVGELGLGISGGQKQRILIARALYRKPSILFMDEATSHLDLRNESVINQSISALSITRIIVAHRPSTIASADRVIDLAQSKPVTTA

SEQ ID NO: 133 - protein, 707 aa, Klebsiella variicola
MTNELFKTVVAKLNFSLINKTPVILQSESSECGIACLAMVCGYYGLNIDLFNFRQRFGSPSQGSSLMSLSKTAEHAGLKSRALALDLDEIKQLKLPCVIHWGMNHYVVLTKVRKNGFIIHDPSLGKRIIGISEMSNNFTGVALELWPDHNFQQEEAKSRLRLLDLMHKIVGLKSALIKIFAFSVVVEAIGLLLPIGTQLVTDHVIMAHDESLLSVICIGLIFFTLFRTFISMLRAWTSLTLNTLTNIQWKTTLFDHLASLPLSFFEKRHLGDIQSRFSSLDTIRSIFTNSIVTGIIDSIMTIGLLVMLTLYGGWLVWVVVGFTVCYAIMRFATYGFYRRVTEEQVVKGARANSHFMESLYGIATIKALNLKGRRSQHWLNLNVDASNAGIKQTRFDMMYGGINTFITSIDQVAVLWLGAIMVIDNEMTLGMFMAFNAYRGQFSQRASNLIDLVMQLRMLSLHNERLSEIAFSEPEKELPSRRVFTENTGVQLEVRNLSYQYDPFSQPIFTNLNITIAPGESVALIGPSGVGKTTLLKVMCGLLSPDSGEILADNLEIKKIGLNNYRLGIACVLQEDRLFSGSLIDNISGFDDNVDLDFVMECAKRCNIHDEIMKMAMGYETIVGELGLGISGGQKQRILIARALYRKPSILFMDEATSHLDLRNESVINQSISALSITRIIVAHRPSTIASADRVIDLSQSKSVTTA

SEQ ID NO: 134 - protein, 704 aa, Kosakonia oryzae
MNKDILDSIFKRINFSIKNKVPVIIQSEATECGNACLAMICGYYGKDIDLFNFRTHYGSPSQGATLTDLNNVALKTGLKTRALSLDIDEIKELRLPCVLHWSLNHFVVLVAVKGKRFVINDPAMGRRIVHQKEMSENFTGIALEAWPDSHFKQEKQRSRLKLLDLMRNIVGLKSALSKIFMLSVVIEAINLLMPMGTQLVTDHVITAHDSNLLLVICAGLLFFTIFKTAVSMIRAWVSLKLNTLTDIQWKTSFFDHLLSLPLAFFEKRNLGDIQSRFSSLDTIRATFTNNIVMGTIDSIMTVGVLVMMSLYGGWLVWVVLGFTVCYAAMRIITYKFYRTISEELIVKRARSGSHFMETLYGIATIKSLNLKNRRSQHWLNTNIDVSNASLRQTRFDMLFGGINTFINSVDQVVILWLGALMVMDNTMTIGMFMAFNAYRGQFAQRASSLIDLAMQFKMLSLHNERISEIVFSKPEAESPPRKVFENSAGVPLQVQNLSYQYDKLTKPVFSNVNISVAAGESVALIGASGIGKTTLLKVMSGLLTPDHGDIFVGDFDVKKIGVNNYRACTACVLQEDRLFSGSIADNISGFEDNADQQLIVECAIKCNIHEEIMRMPMGYETIIGELGSGISGGQKQRLLIARALYQKPNILFMDEATSHLDANNERMINNAIESLNITRIIVAHRPSTIACADRVIDLSKISSL

SEQ ID NO: 135 - protein, 704 aa, Kosakonia radicincitans
MNKAIIDTIFKRINFSIRNKVPVIIQSEAAECGNACLSMICGYYGKNIDLFNFRNRYGSPAQGATLTDLNQVAQTTGLKTRALSLDMDEIKELRLPCVLHWSLNHFVVLVAIKGKRFVINDPAMGRRVIHQKEMSENFSGIALEAWPDSHFTQEKQRSRLNLLDLMCNIVGLKSALSKIFMLSIVIEAVNLLMPMGTQLVTDHVITAHDNNLLLVICAGLLFFTLFKTAVSMIRAWVSLKLNTLTDIQWKTSFFDHLLSLPLAFFEKRNLGDIQSRFSSLDTIRATFTNNIVMGTIDSIMTVGVLIMMSLYGGWLVWVVLGFTVCYAAMRIITYKFYRTISEELIVKRARSGSHFMETLYGIATIKSLNLKNRRSQHWLNTNIDVSNASLRQTRFDMLFGGINTFINSVDQVVILWLGALMVMDNTMTIGMFMAFNAYRGQFAQRASSLIDLAMQFKMLSLHNERISEIIFSKPEAESPPRKVFEDSAGVPLQVQNLSYQYDKLTRPVFSNVNITVAAGESVALTGASGIGKTTLLKVMSGLLTPDQGDIFVGDFDVKKIGVNNYRACTACVLQEDRLFSGSIADNISGFEDNADRQLIIECAIKCNIHEEIMRMPMGYETIIGELGSGISGGQKQRLLIARALYQKPNILFMDEATSHLDANNERMINNAIESLNITRIIVAHRPSTIACADRVIDLSKISSL

SEQ ID NO: 136 - protein, 695 aa, Mannheimia haemolytica
MTGIKQLKFDFRRRIPVILQTETAECGLACLAMMLGYYDKNTNLFELRSQYGTSSRGVTLHTLISIANDCGLITRPLSLELDEVPQLRLPCILHWDFNHFVVLTHASEKQFIIHDPAFGKRKLSRAEFSNHFTGVALEVWSEVKFEKSNNENTISLYETLKHISGIKGALVKIFALSMLIELIALLMPIGTQLVMDHVMQAKDQSLLLIICIGLFLFTLFRTMVSMFRAWVSLKMNYLIDFQWTASFFSHLLKLPLDFFEKRQVGDIHSRFSSLKTIQKTLTSSIITTIIDTIMIISLIIMMVLYGGWLFWVVLAFSFIYFILRMVTYLVYRQLNEEQIIKGAKAHSHFMETLYGITTLKSLGLENKRQEQWMSLNADAFNTSIKVTKFDMLFSGIHTFISTIEQILILWIGATMVIENVMTLGMFVAFNAYRGSFSSRMGNLIDIIFSLKILSLHRERIADIALNEVEDESKYQELRFEHNQTDIEVKDLCFKYDPFSKNVLENVNLVIRSGESVAITAPSGYGKTTLLKLIAGLLKPTSGSVYFNGIDIYQLGLSQYRSQIACVLQEDKFFSGSILENIVSFESNYDREWAIECSKLANIHNDIMAMPMNYETLLGELGNNLSGGQRQRLFIARALYKKPKILLMDEATSHLDEENEKLVNQAISRMKITRIIIAHRQSTIQSADRIIALNDR

SEQ ID NO: 137 - protein, 704 aa, Morganella morganii
MRRLSLKNLLEKLDLSLRQRIPVIHQTESSECGLASLAMISAHYGKSIDLISLRQQFNLSARGATLAGLTGMAAELGMTTRALSLDMDDLPNLRLPCILHWDFNHFLVLVKISGNKFILHDPAYGRRVVGIEEMSRNFTGVALEAWPGSTFTRQEVVKKLSLRKLIGNIHGLKKALLKIFAFSVVIEAIGLIMPVGTQLVMDHAIPAGDQGLLSLICVGLLFFILLKAAVSMCRAWASVIMETLINVQWQSGLFTHLLRLPLSYFERRKLGDIQSRFGSLDILRTTFTTSVVGAIMDGIMLIGVFIMMILYGGHLTWIVLGFTAVYIGLRLATYRYYRQLSEESLIKEARAGSYFMESLYGITTVKTQGMSDRRGNHWLNLKVDSINTGIRLTKMNLMFGGVNIFIMACDQIAILWIGAGLVIDNAMTLGMFVAFSAYREQFSDRAASIIEFLLQLRIMSLHNERISDIALNEQENKKPDVPYEPQMQAAGLETRNLAYSYDSQSVPIFRDINLSVAPGESVAITGPSGSGKTTLMKVLCGLFEPSDGKVFIDNIEIRQLGVNNYHKMIACVMQDDKLFSGSIRENICGFTENPDEQLMIACAQASYIHNVIISMPMGYETLIGELGEGLSGGQKQRIFIARALYRKPRILFMDEATSALDKESESYVNQAIRQLPITRIIIAHRESTIESADRIISLGGKNNK

SEQ ID NO: 138 - protein, 704 aa, Morganella morganii
MRRLSLKNLLEKLDLSLRQRIPVIHQTESSECGLASLAMISAHYGKSIDLISLRQQFNLSARGAALAGLTGMAAELGMTTRALSLDMDDLPNLRLPCILHWDFNHFLVLVKISGNKFILHDPAYGRRVVGIEEMSRNFTGVALEAWPGSTFTRQEVVKKLSLRKLIGNIHGLKKALLKIFAFSVVIEAIGLIMPVGTQLVMDHAIPAGDQGLLSLICVGLLFFILLKAVVSMCRAWASVIMETLINVQWQSGLFTHLLRLPLSYFERRKLGDIQSRFGSLDILRTTFTTSVVGAIMDGIMLIGVFIMMILYGGHLTWIVLGFTAVYIGLRLATYRYYRQLSEESLIKEARAGSYFMESLYGITTVKTQGMSDRRGNHWLNLKVDSINTGIRLTKMNLMFGGVNTFIMACDQIAILWIGAGLVIDNAMTLGMFVAFSAYREQFSDRAASIIEFLLQLRIMSLHNERISDIALNEQENKKPDVPYEPQMQAAGLETRNLAYSYDSQSAPIFRDINLSVAPGESVAITGPSGSGKTTLMKVLCGLFEPSGGKVFIDNIEIRQLGVNNYHKMIACVMQDDKLFSGSIRENICGFTENPDEQLMIACAQASYIHNVIISMPMGYETLIGELGEGLSGGQKQRIFIARALYRKPRILFMDEATSALDKESESYVNQAIRQLPITRIIIAHRESTIESADRIISLGGKNNK

SEQ ID NO: 139 - protein, 685 aa, Pantoea sp.
MPVLLQTEAAECGLVSLAMIASWYQLPADLRFLRQQSGISSRGATLRTVIDTAAVAGLKSRALRLEMDNLRALKLPCILHWDLNHFVVLVAVKRNRVVIHDPAAGHRVLGMQDVSLHFTGVALELWPSGEMPKTPPARPRIPLWSLTGRIAGLGRTLTKLFCYSLLIESVSLLLPVGMQMVLDHAIPASDAGLLTLICLGLVFFVLFRTGASMLRGWTSLVMSTLITVQGKARLFDHLLALPTEWFEKRKLGDIQSRFTSLETLQTTLTSNVVSGIMDSIMTIGLLIMMLLYGGNLLWVVLAFTLLWVLFRLGTYQAWRQTSEEQIVRNARSGSHFMETLYGIHTIKALGLPALRAQHWFTLNVESANTTIRKTRFEMLFTGGNTLIAALQQITLLWLGANAVIAGHMTLGMFVAFSTYRGQFAERTAALLDNVLQLRMLSLHKERVADIILTEPETTPGEPVHPLLNQGEPAALAVKGLTFRHDPFSPLLFNALDLEIAARECVAITGPSGRGKTTLLKIMAGLLKPTAGEVYVNGIDIRASGIANWQACIACVLQEDTLLAGSVAENICSFDGQPDDVWIQECARLSQIHDDILAMPMGYQTLISELGGSLSGGQKQRLLIARALYRRPQVLFLDEATSHLDVENESRINAAIRGLNITRVMIAHRPSTIASADRVIDMAEWS

SEQ ID NO: 140 - protein, 704 aa, Serratia inhibens
MNKTYEKIVSKLNLAIKRNVPQILQTEAAECGLASLAMVCGYYGMHIDLFALRQKHDISSRGATLGSLITIAENLNLKTRALSLSLDEIRQVKTPCILHWDMNHFVVLVNVRGGRITLHDPAFGRRVIGLQEFSLHFTGIALEVWPSSDFSPVVQQSRLHFRKLLKNVTGLKSALGKIFALSLVIEAINLLMPVGTQLVMDHVIQAGDHNLLVLICVGLLFFIIFRTVVSMFRSWISIVMGALIDIQWKAGLFDHLMRLPLAYFEKRKLGDIQSRFGSLDALRTTFTTSVVSSIIDGIMSIGVFIMMFMYGGWLVWVVTGFTLLYILLRLSTYRYYRQASEEQLVKAAKASSHFMETLYSIATLKSLGLGGTRSQFWLNLNIDTTNANIRVTKLDMMFGGVNAFLTACDQIIILWLGASLVIDNQMTLGMFVAFNAYRGQFSDRASNLIDMVLRLRMLNLHNERLADIVLSTPEEDKPYRKICAPNEAVTFEVRDLYYQYDSLSRPVIPGLNLNIAAGESVAIVGPSGVGKTTLMKLMCGLLEPTGGRLLINGINISDIGINNYRECIACVLQEDKLLAGSIAENICSFDPKPDQDLIVECTKHCNIHNDIIRMPMGYETLVGELGGSLSGGQKQRILIARALYRRPSILFMDEATSHLDLDNETYINNAISALNITRVIIAHRPSTISSADRVITLKSSIQEE

SEQ ID NO: 141 - protein, 699 aa, Serratia marcescens
MTESYFDTLKGKLNLSLRRKVPQILQTEASECGLASLAMVCHYHGLQIDLFNLRSRYGFSSRGATLSALIDIASALKLQSRALSLNIDELKALKMPCILHWDMKHFVVLVSVSRGRAVIHDPAFGRKVLGLHELSRHFTGVALELWPDSEFQPIKQQSRLRFRKLMSNVRGLKGALLKIFCLSIVIETVNLLLPVGTQLVMDHVILAGDHDLLALICIGLLFFILFRTGVSMLRSWISIVMGALIDVQWKAGVFDHLMKLPLSFFEKRKLGDIQSRFGSLDTIRATFTTSIVSSIIDGIMSVGVFIMMLLYGGWLVWVVLGFTAIYVILRLSTYQYYRQLSEEQLVKGARAGSHFMETLYSVSTLKALGLSETRSQYWLNLNVETINAGIKLTKLNMMFGGLGAFIATCDQVIILWLGAALVIDNQMTIGMFIAFNAYRGQFSERASSLIDMMLQLRMLSLHNERVADIVLSSPESQMPARQLFTKGKAAELQVRNLRYQYDRLAKPIIADLNLSIAAGESVAIVGPSGAGKTTLMKLMCGLLSPDEGAVLVDGMDISNIGVNNYRQCIACVLQDDKLLAGSIAENISGFDAEMDLKRIEACARRCNMHDDIQNMPMGYETLIGELGGSLSGGQKQRLLIARALYRRPSILFMDEATSHLDLDNETHINRAISQLNITRVIIAHRPSTIASADRVIRLG

SEQ ID NO: 142 - protein, 699 aa, Serratia marcescens
MTESYFDALKGKLNLSLRRKVPQILQTEASECGLASLAMVCHYHGLQIDLFNLRSRYGFSSRGATLAALIDIASALKLQSRALSLNIDELKALKMPCILHWDMKHFVVLVSVSRGRAVIHDPAFGRKVLGLHELSRHFTGVALELWPDSEFQPIKQQSRLRFRKLMSNVRGLKGALLKIFCLSIVIEAVNLLLPVGTQLVMDHVILAGDHDLLALICIGLLFFILFRTGVSMLRSWISIVMGALIDVQWKAGVFDHLMKLPLSFFEKRKLGDIQSRFGSLDTIRATFTTSIVSSIIDGIMSVGVFIMMLLYGGWLVWVVLGFTAIYVILRLSTYQYYRQLSEEQLVKGARAGSHFMETLYSVSTLKALGLSETRSQYWLNLNVETINAGIKLTKLNMMFGGLGAFIATCDQVIILWLGAALVIDNQMTIGMFIAFNAYRGQFSERASSLIDMMLQLRMLSLHNERVADIVLSSPESQMPARQLFTKGNAAELQVRNLRYQYDRLAKPIIADLNLSIAAGESVAIVGPSGAGKTTLMKLMCGLLSPDEGAVLVDGMDISNIGVNNYRQCIACVLQDDKLLAGSIAENISGFDAEMDLKRIEACARRCNMHDDIQNMPMGYETLIGELGGSLSGGQKQRLLIARALYRRPSILFMDEATSHLDLDNETHINRAISQLNITRVIIAHRPSTIASADRVIRLG

SEQ ID NO: 143 - protein, 707 aa, Escherichia coli
MNNNATSPLNTLLNKLEIGLRRRIPVVHQTESSECGLACLSMICGHYGRHIDLSTLRRQFNLSALGTTLAGITEIGSQLGMETRAFSLDLNELSVLKLPCILHWEFSHFVVLVSVRKNHFVLHDPARGRRTVGLAEMSQCFTGVALEVWPGTEFVQETMKNRVVLRTLFRSIYGLRSTLTKIFCFSLVIEAVGLVIPVGTQLVMDHAIPAGDRGLLSLICVGLMFFILLRTAVSMIRSWSSLVMETLINVQWQSGLHRHLLQLPLAYFERRKMGDIQSRFSSLDTLRTTFTTSVVGAIMDSIMVSGVLAMLVLYGGWLTTIVLGFTIIYVLIRLLTYNYYRQLSEESLIREARASSYFMETLYGIATIKMQGMGERRGRHWLNLKIDAINTGIRLARMDMLFSGINTFVAACDQVVILWLGTSLVIDNQMTIGMFVAFGVFRGQFSDRVGSLTNFLLQLRMMSLHNERIADIAMNEREARKPDTAMKADMYPVALETQDLSFRYDSQSAPVFSNLNISIKPGESVAITGASGSGKTTLMKVLCGLLVPESGRVMIDGTDIRSLGVNNYHKIISCVMQDDRLFSGSIRENICGFTENIDEAWMVECARASFIHDVIIKMPMGYDTLIGELGEGLSGGQKQRIFIARALYRRPGILFMDEATSALDTESEYYVNQAIKQLNITRIIIAHRETTVKSADRIILLEAPARI

SEQ ID NO: 144 - protein, 702 aa, Acinetobacter calcoaceticus
MLNFSLKRKVPVILQHESAECGLACLAMIVNYYGKSISIITLRNKYKISLQGTSLNMLMIISDDLQLQTRPIKIDIENLENINLPCILHWEFNHFVILTKINHNNYIIHDPAIGVRKINSEDFSKSFTGIALEIWPKPNFNSQNNEYKIKKSNFNNFFNEIIGIKSVLVQIFFISLSLELVILLNPQLLQWIIDQIIPTQNESSLSILALSFVFILAFQVILDLARENLNIFMKFNISLKWQANIVSKILDLPVDFFEKRSIGDIASKFGSINTILNTITSSFMTAIFDGIMSILILSLLLFYSPLLTSIVFIFTILYLVIKIVFYYPQKYKNEERIIHTAKQNSHFLESIRGVKTIKTFNAQIERKSIWFSLLTNQINSEIQVEKINIMTKEFRIFLFGLENIVIIYLATLLVLKNELSVGSLVAFVVYKTLFTNRITALIDKLSEIGTIKLHNDRLRDISLTEPEIIYPQEFMEINSNKLSIKVKNISFSYSNYEKSILNNITFEIPYGQSVAITGASGSGKSTLLNILLGILAPTTGDVFYGEHKIKELGLYNIRSLVGTVLQEDVLFSGSIADNISFFDASSNLDWIQECAKMAAIHEEIINTSMGYNTLIGEMGTVLSGGQKQRILLARALYKRPKILFLDEATSNLDIYKEFEVNNLIKKLKITRFIIAHRQETINSADRVLIIEDGILRSDIMNS

SEQ ID NO: 145 - protein, 702 aa, Bacteria sp.
MKSEVVSVLKNKLDLGLKSRFPMIHQTESSECGLACLAMICGYYGKNIDIFTLRQKFNLSSRGTDLASLNDIAVKMHMSTRAVSVELDELHSVKLPCILHWDFNHFVVLTKIKNGKFTIHDPSVGIVKVAKSELSKKFTGIILEIWPDNNFLKENVKNKISLRAMLENTHGLYSALTKIFFLSIIIESINLVMPVATQLVMDHAIPANDSGLLTLICFGLVFFVALRTITGLIRSWTVLIMSSFINAQWQKGLLQHLLKLPLEYFERRKFGDIQSRFGSLNTLQETFTTSIVGAIIDSIMIIGLLIMLVLYGGWLTWFVILFTCLYVFIRVLTYNRYRQLSEESLIKDARANSFFMETLYGIATVKVQGLNKRREENWFNLEVDSINTGIKVSRLNLFFGGINTLIATMEQILILWLGASLVISGNMTIGMFVAFSSYRGQFSDRIASFIDFLLRLQIMSLHNERVSDIALTEVSPFKDDIPVKNDMCPVSLSAKNISYKYDSYASYTLRHLNIEIQPGEHVAITGASGTGKTTLLKVLCGLFPASEGNILVDGIDIKELGINNYQKLIACVLQDDRLFSGTLRDNICSFSTQIDDNWLIECARFAQIHDDIERMPMGYDTLIGELGEGLSGGQKQRIFIARAIYKKPSILFMDEATCSLDNTNEQYINDAIKSLNITRIVIAHRKSTINSADRIITLQENL

SEQ ID NO: 146 - protein, 706 aa, Enterobacteriaceae sp.
MNNKLFETIIAKLNFSLIKKTPVILQSESAECGIASLAMICGHYGLDIDLFNFRQRFGSPSQGASLLHLSKTAERAGLKNRALSLDLDEIKQLKLPCIIHWGMNHYVVLTKVRKSSYVVHDPALGKRIIGLQEMSNNFTGVALELWPDQNFQQEKAKARLRLLDLMRNIVGLKSALIKIFAFSVVIEAIGLLLPIGTQLVTDHVIMAHDQSLLSVICIGLVFFTLFRTFVSMLRAWTSLTLNTLTNIQWKTTLFDHLTSLPLSFFEKRHLGDIQSRFTSLDTIRSTFTNSIVSGIIDSIMTIGLLIMLTLYGGWLVWVVVGFTLCYAIMRLATYRFYRRVAEEQVIKGARSSSHFMESLYGISTIKALNLKERRSQHWLNINIDACNAGIKQTRFDMMFSGINTFISSIDQVAILWLGAIMVIDNNMTLGMFMAFNSYRGQFSQRASNLIDLFMQLRMLSLHNERLSEIVFSEPEKELPARRVFDENSGVKLEIKNLSYQYDPFSQPIFSNFNLQVEPGESIALVGPSGVGKTTLLKVMCGLLSPTSGEVIADNLDIYKIGLNNYRLGTACVLQEDRLFSGSISDNISGFEDNADEELIVECARRSNIHDEIMKMPMGYETMIGELGLGISGGQKQRLLIARALYRKPSILFMDEATSHLDLKNESAINQSIASLSITRIIVAHRPSTIASADRIIDLSQYGKQAS

SEQ ID NO: 147 - protein, 711 aa, Halomonas sp.
MNLAELPKRLLGSMKKGTPVIIQSEAAECGLACMAMVASYHGHALDLPAMRRHFATSLKGMALRDLLEIGNGLHLSTRAVRLELEDLKQLKLPCILHWDHNHFVVLTRVGSRGVTIHDPATGSRTLTMSEVSSHFTGVALEAWPTEFFAKKDERERIHVTEMIKRTAGIGRAALQILSISVLLELVTIAMPIGFQMIIDEVIVASDYDLLTLITIAFCFFLALQVLASFIRSWTSMLIGSGLVLQWKAGLFDHLMRLPLSYFEKRHVGDIVSRFGSVDSIQQTLTTTAITTLLDGGMSIALIIMMWLYGGWLAGIAMIAVVLYTILRVATYFRYRSLSEEAIVYAAKENTHFMESLRGMASLKVMNLKERRRSVWVNYLVDRINANLKVQKFDVVFQTASKLIFGIDRIIIIYLGAKAILGDTLSVGMLIAFLSYKDQFAARMDSFVTTILSLRMLSLHGERIADIALSEPEESQIDAAEQAYDTAGTEKLGRLEIENVSFRYSDNEPDVLKNINITIEQNECVGISGPSGTGKTTLMKIISGLMLPNEGEVRIDGKTLSTLGLNAYRDRIGCVLQDDRLFAGSIAENICSFDPNPDLRLMQQCAMLAAIHTEIAKMPMGYETFVGDMGSTLSGGQRQRIILARALYRRPSLLLLDEATSNLDPENEAAINAAIKQLSITRIVIAHRPSTLAMTDRVIDLKELQDQASNAQ

SEQ ID NO: 148 - protein, 706 aa, Klebsiella sp.
MNNKLFETIIAKLNFSLIKKTPVILQSESAECGIASLAMICGHYGLDIDLFNFRQRFGSPSQGASLLHLSKTAERAGLKNRALSLDLDEIKQLKLPCILHWGMNHYVVLTKVRKSSYVVHDPALGKRIIGLQEMSNNFTGVALELWPDQNFQQEKAKARLRLLDLMRNIVGLKSALIKIFAFSVVIEAIGLLLPIGTQLVTDHVIMAHDQSLLSVICIGLVFFTLFRTFVSMLRAWTSLTLNTLTNIQWKTTLFDHLTSLPLSFFEKRHLGDIQSRFTSLDTIRSTFTNSIVSGIIDSIMTIGLLIMLTLYGGWLVWVVVGFTLCYAIMRLATYRFYRRVAEEQVIKGARSSSHFMESLYGISTIKALNLKERRSQHWLNINIDACNAGIKQTRFDMMFSGINTFISSIDQVAILWLGAIMVIDNNMTLGMFMAFNSYRGQFSQRASNLIDLFMQLRMLSLHNERLSEIVFSEPEKELPARRVFDENSGVKLEIKNLSYQYDPFSQPIFSNFNLQVEPGESIALVGPSGVGKTTLLKVMCGLLSPTSGEVIADNLDIYKIGLNNYRLGTACVLQEDRLFSGSISDNISGFEDNADEELIVECARRSNIHDEIMKMPMGYETMIGELGLGISGGQKQRLLIARALYRKPSILFMDEATSHLDLKNESAINQSIASLSITRIIVAHRPSTIASADRIIDLSQYGKQAS

SEQ ID NO: 149 - protein, 707 aa, Klebsiella sp.
MNKELFKTITDKLNFSFRHKTPVILQSEAAECGIACLAMVCGFYGLNIDLFNFRHRYGSPSQGVTLMSLSKTAEHAGLKSRALSLDLDEIKQLKLPCVIHWGMNHYVVLTKVRKSSYIVHDPALGKRVIGIQEMSNNFTGVALELWPDQNFQQEQAKSRLRLLDLMRNIVGLKSVLLKIFAFSVVVEAIGLLLPIGTQLVTDHVIMAHDQSLLSVICIGLVFFTLFRTFISMLRAWISLTLNTLTNIQWKTTLFDHLTSLPLSFFEKRHLGDIQSRFSSLDTIRSTFTNSIVTGIIDSIMTVGLLIMLTLYGGWLVWVVVGFTLCYVIMRFATYSFYRRVAEEQVIKGARSSSHFMESLYGISTIKALNLKERRSQHWLNINIDACNAGIKQTRFDMMFGGINTFITSIDQVAVLWLGAIMVIDNNMTLGMFMAFNAYRGQFSQRASSLIDLIMQLRMLSLHNERLSEIVFSEPEKELPSRRVFAENTGVKLEVKNLSYQYDPFSQPIFSNLNITVEPGESIALVGPSGVGKTTLLKVMCGLLSPTSGDVIADNLDIQKIGLNNFRHGTACVLQEDRLFSGSLIDNISGFEDNADMDFIVECARRCNIHDEIMKMPMGYETIVGELGLGISGGQKQRLLIARALYRKPSILFMDEATSHLDLKNESMINQSISGLSITRIIVAHRPSTIASADRIIDLSQLRTLAPA

SEQ ID NO: 150 - protein, 702 aa, Klebsiella sp.
MKSEVISLLKNKLDLGLKSRFPMIHQTESSECGLACLAMICGYYGKNIDIFTLRQKFNLSSRGTDLASLNDIAVKMHMSTRAVSVELDELHSVKLPCILHWDFNHFVVLTKIKNGKFTVHDPSVGIVKVAKSELSKKFTGIVLEIWPGNSFLKENVKNKISLRAMLENTHGLYSALTKIFFLSIIIESINLVMPVATQLVMDHAIPANDSGLLTLICFGLVFFVALRTITGLIRSWTVLIMSSFINAQWQKGLLQHLLKLPLEYFERRKIGDIQSRFGSLNTLQETFTTSVVGAIIDSIMIIGLLIMLVLYGGWLTWFVILFTCLYVFIRVLTYNRYRQLSEESLIKDARANSFFMETLYGIATVKVQGLNKRREENWFNLEVDSINTGIKVSRLNLFFGGINTLIATMEQILILWLGASLVISGNMTIGMFVAFSSYRGQFSDRIASFIDFLLRLQIMSLHNERVSDIALTEVTPFKDDIPVKNDMCPVSLSAKNISYKYDSYASYTLRHLNIEIQPGEHVAITGASGTGKTTLLKVLCGLFPASEGNIFVDGIDIKELGVNNYQKLIACVLQDDRLFSGTLRDNICSFSTQIDDNWLIECARFAQIHDDIERMPMGYDTLIGELGEGLSGGQKQRIFIARAIYKKPSILFMDEATSSLDHTNEQYINDAIKSLNITRIVIAHRKSTINSADRIIALQENL

SEQ ID NO: 151 - protein, 706 aa, Klebsiella sp.
MNNKLFETIIAKLNFSLIKKTPVILQSESAECGIASLAMICGHYGLDIDLFNFRQRFGSPSQGASLLHLSKTAERAGLKNRALSLDLDEIKQLKLPCILHWGMNHYVVLTKVRKSSYVVHDPALGKRIIGLQEMSNNFTGVALELWPDQNFQQEKAKARLRLLDLMRNIVGLKSALIKIFAFSVIIEAIGLLLPIGTQLVTDHVIMAHDQSLLSVICIGLVFFTLFRTFVSMLRAWTSLTLNTLTNIQWKTTLFDHLTSLPLSFFEKRHLGDIQSRFTSLDTIRSTFTNSIVSGIIDSIMTIGLLIMLTLYGGWLVWVVVGFTLCYAIMRLATYRFYRRVAEEQVIKGARSSSHFMESLYGISTIKALNLKERRSQHWLNINIDACNAGIKQTRFDMMFSGINTFISSIDQVAILWLGAIMVIDNNMTLGMFMAFNSYRGQFSQRASNLIDLFMQLRMLSLHNERLSEIVFSEPEKELPARRVFDENSGVKLEIKNLSYQYDPFSQPIFSNFNLQVEPGESIALVGPSGVGKTTLLKVMCGLLSPTSGEVIADNLDIYKIGLNNYRLGTACVLQEDRLFSGSISDNISGFEDNADEELIVECARRSNIHDEIMKMPMGYETMIGELGLGISGGQKQRLLIARALYRKPSILFMDEATSHLDLKNESAINQSIASLSITRIIVAHRPSTIASADRIIDLSQYGKQAS

SEQ ID NO: 152 - protein, 704 aa, Klebsiella sp.
MNKNIFKTITDSLNFSLRKKIPVILQSESAECGIACITMISCYYGLNIDLFNFRKRYGSPSQGFTLLSLSNNAERAGLKSRALSLDLDEINQLKLPCIIHWGMNHYVVLTKVRRSSFIIHDPALGKRVIGLQEMSNNFTGIALELWPDQNFQQEKAKSRLRLLDLMRNIVGLKSTLIKIFAYSVVIEAIGLLIPIGTQMVTDHVIMAHDQSLLAVICIGLVSFTLFRTFISMLRAWTSLTLNTLTNIQWKTTLFDHLTSLPLSFFEKRHLGDIQSRFSSLDIIRSTFTNSIVTGIIDTIMTIGLLIMLTLYGGWLVWVVVGFTLCYAIMRLATYRFYRRVSEEQVIKGARSSSHFMESLYGIATIKALNLKERRSQHWLNLNIDACNAGIKQTRFDMMFSGINTFISSIDQVAVLWLGALAVINNDMTLGMFMAFNAYRGQFSQRASSLIDLFMQLRMLSLHNERLSEIAFSEPEKDMPTRRIFDKDEGVKLEVKNLSYQYDPFSQPIFSNLNIIIEQGESVALIGPSGIGKTTLLKVMCGLLSPSAGDVLVDNLNISKIGLNNYRLSTACVLQEDRLFSGSILENISGFEDNVDEEFVIECARHSNIHDEIMKMPMGYETLIGELGSGISGGQKQRLLIARALYRKPCILFMDEATSHLDLNNEASINNAISKLSITRIIVAHRPSTIASADRVIDLSESASK

SEQ ID NO: 153 - protein, 706 aa, Klebsiella sp.
MNKKLFETIIAKLNFSLIKKTPVILQSESAECGIASLAMICGHYGLDIDLFNFRQRFGSPSQGASLLHLSKTAERAGLKNRALSLDLDEIKQLKLPCILHWGMNHYVVLTKVRKSSYVVHDPALGKRIIGLQEMSNNFTGVALELWPDQNFQQEKAKARLRLLDLMRNIVGLKSALIKIFAFSVVIEAIGLLLPIGTQLVTDHVIMAHDQSLLSVICIGLVFFTLFRTFVSMLRAWTSLTLNTLTNIQWKTTLFDHLTSLPLSFFEKRHLGDIQSRFTSLDTIRSTFTNSIVSGIIDSIMTIGLLIMLTLYGGWLVWVVVGFTLCYAIMRLATYRFYRRVAEEQVIKGARSSSHFMESLYGISTIKALNLKERRSQHWLNINIDACNAGIKQTRFDMMFSGINTFISSIDQVAILWLGAIMVIDNNMTLGMFMAFNSYRGQFSQRASNLIDLFMQLRMLSLHNERLSEIVFSEPEKELPARRVFDENSGVKLEIKNLSYQYDPFSQPIFSNFNLQVEPGESIALVGPSGVGKTTLLKVMCGLLSPTSGEVIADNLDIYKIGLNNYRLGTACVLQEDRLFSGSISDNISGFEDNADEELIVECARRSNIHDEIMKMPMGYETMIGELGLGISGGQKQRLLIARALYRKPSILFMDEATSHLDLKNEAAINQSIASLSITRVIVAHRPSTIASADRIIDLSQYGKQAS

SEQ ID NO: 154 - protein, 707 aa, Klebsiella aerogenes
MTNELFEKITAKVNFSILKKTPVILQSEAAECGIACLAMVCGHYGLDIDLFNFRQRFGSPSQGVTLMSLSKTAEHAGLKSRALSLDLDEIRQLKLPCVIHWGMNHYVVLTKVRKSSFVVHDPALGKRIIGIQEMSNNFTGIALELWPDQNFQQEKAKSRLRLLDLMRNIVGLKSALLKIFAFSVVVEAIGLLLPIGTQLVTDHVIMAHDQSLLSVICIGLVFFTLFRTFISMLRAWTSLTLNTLTNIQWKTTLFDHLASLPLAFFEKRHLGDIQSRFSSLDTIRSTFTNSIVSGIIDSIMTIGLLIMLTLYGGWLTWVVVGFTLCYALMRLGTYRFYRRVAEEQVIKGARSSSHFMESLYGISTIKALNLKERRSQHWLNINIDACNAGIKQTRFDMMFSGINTFITAIDQVAVLWLGAIMVIDNEMTLGMFMAFNAYRGQFSQRASSLVDLTMQLRMLSLHNERLSEIVFSEPEQELPSREVFSKDAGARLEVRNLSYQYDPFSQPIFSNLNITVEPGESVALVGPSGVGKTTLLKVMCGLLSPTSGDVFADNLDIQKIGLNNYRHGTACVLQEDRLFSGSLIDNISGFEDNADVDFVVECAKRCNIHDEIMKMPMGYETIVGELGLGISGGQKQRILIARALYRKPSILFMDEATSHLDLRNESVINQSISGLSITRVIVAHRPSTIASADRVIDLSQSRIPMPA

SEQ ID NO: 155 - protein, 703 aa, Klebsiella aerogenes
MNRNHFISSIEEKLDLKFRRRVPVVHQTETSECGLACLAMICGHYGRNIDMIALRRQINLSARGASLASVQQTGIELGMESRALSLDLHELNALRMPCILHWNFNHFVVLVSVRKNRFVLHDPARGRRTVGSDEMSQCFTGVALEVWPGSTFVQETMKSRIGIRALISSVQGIRGVLSKIFCLSLIIEAIGLVMPVGTQLVMDHAIPANDRGLLMLICGVLMFFILGRTLVSVIRAWSSLAMESLINVQWQSGLFRHLLQLPLTYFERRKMGDIHSRFSSLDALRATFTTCVVGAMMDSIMVAGLLLMLILYGGKLTVFVLGFTAVYVFIRILTYSRYRQLSEEALIAGARAGSYFMETLYGIATVKMQGMAERRGSHWLNLKIDAINTGIRLSRLDMFFGGINTFVATCDQVTILWLGISLVMENQMTIGMFVAFGAFRAQFSDRVGSLVNFLLQLRMMSLHNERISDIALNEKEERKPDLGLKATMSPASLEIKALSFRYDSQSAPVFSGLSFRIRAGESVAITGPSGSGKTTLLKVLCGLSEPISGTVLMNGTDIRQLGINNYHRLIACVMQDDRLFSGSIRENICGFTENIDDAWMRECARTSRIHDVILALPMGYETLIGELGEGLSGGQKQRIFIARALYRRPDILFMDEATSALDAECEKDVSLAMKKLNITRIIIAHRETTLQTADRIITLGESN

SEQ ID NO: 156 - protein, 712 aa, Klebsiella huaxiensis
MTSELFNTIISRLNFSLCKKTPVIIQSEASECGIACLSMVCGYYGLEIDLFNFRQRFGSSSQGVTLLELSKVAEQANLKNRALTLDLDEIKQLKLPCILHWGMNHYVVLVAVKRSSFIVHDPALGKRVIGIQEMSNNFTGVAMELWPDNNFRQEKVKSRLRLMDLMKNVVGLKSALTKIFALSVVIEAINLLMPIGTQLVTDHVIIAHDHSLLSVICIGLVVFTLFRTFISMLRAWTSLTLNTLTDIQWKTTLFDHLFSLPLTFFEKRHLGDIQSRFSSLDVIRSTFTNSIVTSIIDIIMTVGLLIMMSLYGGWLVWVVIGFTLCYAVMRFGTYRFYRRIAEERVIKNARSGSHFMESLYGISTIKALNLKERRSKNWLNINVEACNAGIKQTRFDMMFGGINTFISSMDQVVILWLGAVMVIENNMTLGMFMAFNAYRGQFSQRASSLIDLVMQLRMLSLHNERLSEIVFSEPEDEQPSRRIFEINKGVSLAVKDLSYQYDPFSRPIFSELNLTINPGESVALVGPSGIGKTTLLKVMCGLLVPSNGQILADDLDVSKIGLNNYRLSTACVLQDDRLFSGSIADNICSFEDNPDQERIIACAQYCNIHEEIMRMSMGYESIVGELGLGISGGQKQRILIARALYRQPSILFMDEATSHLDLANESFINQSISNLKITRIIVAHRPSTIASADRVIDLSQYFKNASQTASLI

SEQ ID NO: 157 - protein, 707 aa, Klebsiella pneumoniae
MTNEIFTTIISRLNFSLRHKTPVILQSEATECGIACLAMVCGHYGLDIDLFNFRQRFGSASQGVTMMSLSKTADHAGLKSRALSLDLNEIRQLKLPCILHWGMNHYVVLTKVRKSSFIVHDPALGKRVIGNQEMSNYFTGIALELWPDQNFQQEKAKSRLRLLDLMRNIIGLKSTLLKIFAFSVVVEAIGLLLPIGTQLVTDHVIMAHDQSLLSVICIGLVFFTLFRTFISMLRAWTSLTLNTLTNIQWKTTLFDHLASLPLSFFEKRHLGDIQSRFSSLDTIRSTFTNSIVSGIIDSIMTIGLLIMLTLYGGWLTWVVVGFTLCYAIMRFATYRFYRRVAEEQVIKGARSSSHFMESLYGISTIKALNLKERRSQHWLNINIEACNAGIKQTRFDMMFGGINTFITTIDQVAVLWLGAIMVIDNNMTLGMFMAFNAYRGQFSQRASSLIDLCMQLRMLSLHNERLSEIVFSEPEQELPAREVFSADSGAKLEVKNLCYQYDPFSQPIFSNLNITVEPGESVALIGPSGVGKTTLLKVMCGLLSPTSGDVLADNLDITKIGLNNYRHGTACVLQEDRLFSGSLIDNISGFEDNADINFVMECARRCNIHDEIMKMPMGYETIVGELGLGISGGQKQRILIARALYRKPSILFMDEATSHLDLKNESVINQSISALSITRIIVAHRPSTIASADRVIDLSQTKNLAPA

SEQ ID NO: 158 - protein, 707 aa, Klebsiella pneumoniae
MTNELFKTVVAKLNFSLVNKTPVILQSESSECGIACLAMVCGFYGLNIDLFNFRQRFGSPSQGSSLMSLSKTAEHAGLKSRALALDLDEIKQLKLPCIIHWGMNHYVVLTKVRKNAFVIHDPALGKRIIGINEMSNNFTGVALELWPDHNFQQEEAKSRLRLLDLMHKIVGLKSALIKIFAYSVVVEAIGLLLPIGTQLVTDHVIMAHDQSLLSVICIGLIFFTLFRTFISMLRAWTSLTLNTLTNIQWKTTLFDHLTSLPLSFFEKRHLGDIQSRFSSLDTIRSIFTNSIVTGIIDSIMTIGLLVMLTLYGGWLVWVVIGFTICYAIMRFATYSFYRRVTEEQVVKGARANSHFMESLYGISTIKALNLKERRSQHWLNLNVDASNAGIKQTRFDMMFGGINTFITSIDQVVVLWLGAIMVIDNEMTLGMFMAFNAYRGQFSQRASNLIDLVMQLRMLSLHNERLSEIAFSEPEKELPSRRVFTENTGVKLEVRNLSYQYDPFSQPIFTNLNITVAPGESVALIGPSGVGKTTLLKVMCGLLSPDSGEILADNLEIKKIGLNNYRLGIACVLQEDRLFSGSLIDNISGFDDSADLDFVMECARRCNIHDEIMKMAMGYETIVGELGLGISGGQKQRILIARALYRKPSILFMDEATSHLDLRNESVINQSISALSITRIIVAHRPSTIASADRVIDLSQSKSVTTA

SEQ ID NO: 159 - protein, 707 aa, Klebsiella pneumoniae
MTNELFKTVVAKLNFSLVNKTPVILQSESSECGIACLAMVCGFYGLNIDLFNFRQRFGSPSQGSSLMSLSKTAKHAGLKSRALALDLDEIKQLKLPCIIHWGMNHYVVLTKVRKNGFVIHDPALGKRIIGINEMSNNFTGVALELWPDHNFQQEEAKSRLRLLDLMHKIVGLKSALIKIFAYSVVVEAIGLLLPIGTQLVTDHVIMAHDQSLLSVICIGLIFFTLFRTFISMLRAWTSLTLNTLTNIQWKTTLFDHLTSLPLSFFEKRHLGDIQSRFSSLDTIRSIFTNSIVTGIIDSIMTIGLLVMLTLYGGWLVWVVIGFTICYAIMRFATYSFYRRVTEEQVVKGARANSHFMESLYGISTIKALNLKERRSQHWLNLNVDASNAGIKQTRFDMMFGGINTFITSIDQVVVLWLGAIMVIDNEMTLGMFMAFNAYRGQFSQRASNLIDLVMQLRMLSLHNERLSEIAFSEPEKELPSRRVFTENTGVKLEVRNLSYQYDPFSQPIFTNLNITVAPGESVALIGPSGVGKTTLLKVMCGLLSPDSGEILADNLEIKKIGLNNYRLGIACVLQEDRLFSGSLIDNISGFDDSADLDFVMECARRCNIHDEIMKMAMGYETIVGELGLGISGGQKQRILIARALYRKPSILFMDEATSHLDLRNESVINQSISALSITRIIVAHRPSTIASADRVIDLSQSKSVTTA

SEQ ID NO: 160 - protein, 706 aa, Klebsiella pneumoniae
MNNKLFETIIAKLNFSLIKKTPVILQSESAECGIASLAMICGHYGLDIDLFNFRQRFGSPSQGASLLHLSKTAERAGLKNRALSLDLDEIKQLKLPCILHWGMNHYVVLTKVRKSSYVVHDPALGKRIIGLQEMSNNFTGVALELWPDQNFQQEKAKARLRLLDLMRNIVGLKSALIKIFAFSVIIEAIGLLLPIGTQLVTDHVIMAHDQSLLSVICIGLVFFTLFRTFVSMLRAWTSLTLNTLTNIQWKTTLFDHLTSLPLSFFEKRHLGDIQSRFTSLDTIRSTFTNSIVSGIIDSIMTIGLLIMLTLYGGWLVWVVVGFTLCYAIMRLATYRFYRRVAEEQVIKGARSSSHFMESLYGISTIKALNLKERRSQHWLNINIDACNAGIKQTRFDMMFSGINTFISSIDQVAILWLGAIMVIDNNMTLGMFMAFNSYRGQFSQRASNLIDLFMQLRMLSLHNERLSEIVFSEPEKELPERRVFDENSGVKLEIKNLSYQYDPFSQPIFSNFNLQVEPGESIALVGPSGVGKTTLLKVMCGLLSPTSGEVIADNLDIYKIGLNNYRLGTACVLQEDRLFSGSISDNISGFEDNADEELIVECARRSNIHDEIMKMPMGYETMIGELGLGISGGQKQRLLIARALYRKPSILFMDEATSHLDLKNESAINQSIASLSITRIIVAHRPSTIASADRIIDLSQYGKQAS

SEQ ID NO: 161 - protein, 707 aa, Klebsiella pneumoniae
MTNEIFTTIISRLNFSLRHKTPVILQSEATECGIACLAMVCGHYGLDIDLFNFRQRFGSASQGVTMMSLSKTADHAGLKSRALSLDLNEIRQLKLPCILHWGMNHYVVLTKVRKSSFIVHDPALGKRVIGNQEMSNYFTGIALELWPDQNFQQEKAKSRLRLLDLMRNIIGLKSTLLKIFAFSVVVEAIGLLLPIGTQLVTDHVIMAHDQSLLSVICIGLVFFTLFRTFISMLRAWTSLTLNTLTNIQWKTTLFDHLASLPLSFFEKRHLGDIQSRFSSLDTIRSTFTNSIVSGIIDSIMTIGLLIMLTLYGGWLTWVVVGFTLCYAIMRFATYRFYRRVAEEQVIKGARSSSHFMESLYGISTIKALNLKERRSQHWLNINIEACNAGIKQTRFDMMFGGINTFITTIDQVAVLWLGAIMVIDNNMTLGMFMAFNAYRGQFSQRASSLIDLCMQLRMLSLHNERLSEIVFSEPEQELPAREVFSADSGAKLEVKNLCYQYDPFSQPIFSNLNITVEPGESVALIGPSGVGKTTLLKVMCGLLSPTSGDVLADNLDITKIGLNNYRHGTACVLQEDRLFSGSLIDNISGFEDNADIDFVMECARRCNIHDEIMKMPMGYETIVGELGLGISGGQKQRILIARALYRKPSILFMDEATSHLDLKNESVINQSISALSITRIIVAHRPSTIASADRVIDLSQTKTLAPA

SEQ ID NO: 162 - protein, 707 aa, Klebsiella pneumoniae
MTNELFKTVVAKLNFSLVNKTPVILQSESSECGIACLAMVCGFYGLNIDLFNFRQRFGSPSRGSSLMSLSKTAKHAGLKSRALALDLDEIKQLKLPCIIHWGMNHYVVLTKVRKNGFVIHDPALGKRIIGINEMSNNFTGVALELWPDHNFQQEEAKSRLRLLDLMHKIVGLKSALIKIFAYSVVVEAIGLLLPIGTQLVTDHVIMAHDQSLLSVICIGLIFFTLFRTFISMLRAWTSLTLNTLTNIQWKTTLFDHLTSLPLSFFEKRHLGDIQSRFSSLDTIRSIFTNSIVTGIIDSIMTIGLLVMLTLYGGWLVWVVIGFTICYAIMRFATYSFYRRVTEEQVVKGARANSHFMESLYGISTIKALNLKERRSQHWLNLNVDASNAGIKQTRFDMMFGGINTFITSIDQVVVLWLGAIMVIDNEMTLGMFMAFNAYRGQFSQRASNLIDLVMQLRMLSLHNERLSEIAFSEPEKELPSRRVFTENTGVKLEVRNLSYQYDPFSQPIFTNLNITVAPGESVALIGPSGVGKTTLLKVMCGLLSPDSGEILADNLEIKKIGLNNYRLGIACVLQEDRLFSGSLIDNISGFDDSADLDFVMECARRCNIHDEIMKMAMGYETIVGELGLGISGGQKQRILIARALYRKPSILFMDEATSHLDLRNESVINQSISALSITRIIVAHRPSTIASADRVIDLSQSKSVTTA

SEQ ID NO: 163 - protein, 707 aa, Klebsiella pneumoniae
MTNELFEKIIAKVNFSLLKKTPVILQSEAAECGIACLAMVCGHYGLDIDLFNFRQRFGSPSQGVTLMSLSKTAEHAGLKSRALSLDLDEIRQLKLPCVLHWGMNHYVVLTKVRKSSFIVHDPALGKRIIGIQEMSNYFTGVALELWPDQNFQQEKAKSRLRLLDLMRNIVGLKSALLKIFAFSVVVEAIGLLLPIGTQLVTDHVIMAHDQSLLSVICIGLVFFTLFRTFISMLRAWTSLTLNTLTSIQWKTTLFDHLASLPLSFFEKRHLGDIQSRFSSLDTIRSTFTNSIVSGIIDSIMTIGLLIMLTLYGGWLTWVVVGFTLCYAIMRFATYRFYRRVAEEQVIKGARSSSHFMESLYGISTIKALNLKERRSQHWLNINIEACNAGIKQTRFDMLFSGINTFITAIDQVAVLWLGAIMVIDNNMTLGMFMAFNAYRGQFSQRASSLVDLCMQLRMLSLHNERLSEIVFSEPEQELPTREVFSADSGAKLEVKNLCYQYDPFSQPIFSNLNITVEPGESVALVGPSGVGKTTLLKVMCGLLSPTSGDVLADNLDITKIGLNNYRLGIACVLQEDRLFSGSLIDNISGFEDNADIDFVMECAKRCNIHDEIIKMPMGYETIVGELGLGISGGQKQRILIARALYRRPSILFMDEATSHLDLKNESVINQSISALSITRIIVAHRPSTIASADRVIDLSQTKTLSPA

SEQ ID NO: 164 - protein, 685 aa, Kosakonia oryzae
MPVLLQTETAECGLVSLAMIADWYNLPTDVRNLRQYGGFSIRGATLRMVMDTAASIGMKSRALQLDMDNLPALKLPCILHWDLNHFVVLAAIRRGKVVIHDPAAGRRIMGMKEVSLHFTGVALELWPAGEMKQEQQPRNRLRLSTLTGRIAGFAITLTKLFCYSILVESCGLLLPAGMQLIMDHAIPANDTSLLTLICLGLLFFVLFRTGISMLRGWTSLVASTLIDIQWKAGLFERMLSLPLAYFEKRRLGDIQSRFSSLGILRTTLTNNVVSAIMDGIMLVGLIVMMLLYGGWLLWIVLAFFLAWGGFRLMTYPAWRRASEELIVREARAGSHFMETLYSINTIKALGLDKQRAQHWINLNVESANATVRKTRYEMLFSGGNILIAALEQIALLWLGAHQVIDGQLTLGMFVAFNTYRGQFADRANNLLNMLLQLRMLSLHKERVADIAHAEAESPLSEHTRQLLAPGEPATLTLHNLSFQYDPFVPPLFSELHIEINAGESVAIIGPSGRGKTTLLKIMAGLLQPTTGEVHINGMNIRTTGLRNYRACIACVLQDDSLLAGSVADNISAFDPQPDEKWMQHCAQLSNIHEDILRMPMGYQTLISELGGSLSGGQKQRLLIARALYRRPSILFLDEATSHLDTVNEAQINTAIKSLRITRVIIAHRPSTIASADRVIDLDRGK

SEQ ID NO: 165 - protein, 729 aa, Escherichia coli
MESINWKVRKQLPVIRQTESAECGLACLAMIACWHGLKTDLSTLRERFNIGIQGMTLQRLIECAASIHLSSRAVRLEPEDLRCLNLPSILHWDMNHFVVLHKVRGNRLYIHDPDRGKITISLLDAGKHFTGVALELTPASDFTPRNERKKIHLRQLTGKTPGLLASMTKIIIFALALEILALGGPLLNQLVIDEVLVAADRSLLYVIIVALLLLSLIQLLLSLARQWATISLSVNFNMQWTARVFHHLVRLPLAWFDARSKGSINARFEAVDIIQQALTTQVLEGILDMLLIVTALCMMLLYSPGMTLIAVIAAIIYGALRALWYPALRQSVEDVWDAGTKESGHFLETLNGIQSLRINGVTIHREAAWLNLNVTRRNTQLRQNRLQMSYELTHTLTESVVSAIILWQGAVEVLDGTFTVGMLVAYLSYQMRFSSSISNLTDNFFSWRMLDVYNERLADIVLTPQEGHQNQHHWANHNETISASQYREHKYDNTHPPLLIEKITFSHKGADKPILDNASLMLFPGEILAITGKSGCGKSTLVKLILGIHTPSEGRINAFGIPHTHSDYFQVRQRIGTVLQDDYLFKGSIADNIMFFSEIRDHEHMRKCASLALIDSDIMAMPMGYQTLLGETGGGLSGGQKQRILLARALYKKPGLLLLDEATSHLDVESEIEISQTLRQLGIPVLLIAHRPETIASADRVLYLRDGHFSEITYRPARTHNINNHPNRR

SEQ ID NO: 166 - protein, 729 aa, Enterobacteriaceae sp.; identical to SEQ ID NO: 165

SEQ ID NO: 167 - protein, 729 aa, Enterobacteriaceae sp.
MESINWKVRKQLPVIRQTESAECGLACLAMIACWHGLKTDLSTLRERFNIGIQGMTLQRLIECAASIHLSSRAVRLEPEDLRCLNLPSILHWDMNHFVVLHKVRGNRLYIHDPDRGKITISLLDAGKHFTGVALELTPASDFTPRNERKKIHLRQLTGKTPGLLASMTKIIIFALALEILALGGPLLNQLVIDEVLVAADRSLLYVIIVALLLLSLIQLLLSLARQWATISLSVNFNMQWTARVFHHLVRLPLAWFDARSKGSINARFEAVDIIQQALTTQVLEGILDMLLIVTALCMMLLYSPGMTLIAVIAAIIYGALRALWYPALRQSVEDVWDAGTKESGHFLETLNGIQSLRINGVTIHREAAWLNLNVTRRNTQLRQNRLQMSYELTHTLTESVVSAIILWQGAVEVLDGTFTVGMLVAYLSYQMRFSSSISNLTDNFFSWRMLDVYNERLADIVLTPQEGHQNQHHWANHNETISASQYREHKYDNTHPPLLIEKITFSHKGADKPILDNASLMLFPGEILAITGKSGCGKSTLVKLILGIHTPSEGRINAFGIPHTHSDYFQVRQRIGTVLQDDYLFKGSIADNIMFFSEIRDHEHMRKCASLALIDSDIMAMPMGYQTLLGETGGGLSGGQKQRILLARALYKKPGLLLLDEATSHLDVESEIEISQTLRQLGIPVLLIAHRPETIASADRVLYLRDGHFSEIKYRPVRTHNINNHPNRR

SEQ ID NO: 168 - protein, 729 aa, Escherichia sp.
MESINWKVRKQLPVIRQTESAECGLACLAMIACWHGLKTDLSTLRERFNIGIQGMTLQRLIECAASIHLSSRAVRLEPEDLRCLNLPSILHWDMNHFVVLHKVRGNRLYIHDPDRGKITISLLDAGKHFTGVALELTPASDFTPRNERKKIHLRQLTGKTPGLLASMTKIIIFALALEILALGGPLLNQLVIDEVLVAADRSLLYVIIVALLLLSLIQLLLSLARQWATISLSVNFNMQWTARVFHHLVRLPLAWFDARSKGSINARFEAVDIIQQALTTQVLEGILDMLLIVTALCMMLLYSPGMTLIAVIAAIIYGALRALWYPALRQSVEDVWDAGTKESGHFLETLNGIQSLRINGVTIHREAAWLNLNVTRRNTQLRQNRLQMSYELTHTLTESVVSAIILWQGAVEVLDGTFTVGMLVAYLSYQMRFSSSISNLTDNFFSWRMLDVYNERLADIVLTPQEGHQNQHHWANHNETISASQYREHKYDNTHPPLLIEKITFSHKGADKPILDNASLMLFPGEILAITGKSGCGKSTLVKLILGIHTPSEGRINAFGIPHTHSDYFQVRQRIGTVLQDDYLFKGSIADNIMFFSEIRDHEHMRKCASLALIDSDIMAMPMGYQTLLGETGGGLSGGQKQRILLARALYKKPSLLLLDEATSHLDVESEIEISQTLRQLGIPVLLIAHRPETIASADRVLYLRDGHFSEIKYRPVRTHNINNHPNRR

SEQ ID NO: 169 - protein, 725 aa, Escherichia coli
MDTLKWTGRKQLPLIRQTESAECGLACLVMMACWHGLQTDLPTLRERFSISTQGMTLQRLIECAAGIRLSSRAVRLEPEDLKSLSLPCILHWNMNHFVVLYKVRGSRLVIHDPDKGKITLSLQDAGKHFTGVALELTPASDFTARDERKKIRLRQLTGRTPGLLAAMSRIIIFALALEILTLSSPLLNQLVIDEVLVAADRSLLTVIIIALLLLSMTQMLLSLARQWASITLSVNFNMQWTARVFHHLVRLPLGWFDARSKGSINARFEAVDTIQEALTTQVLEGILDVLLVITALCMMLLYSPEMTLIAVLAAIIYGVLRALWYPSLRQSAEDAWDAGARESGHFLETLNGILSLRINGVTAHREAAWLNLNIVRRNTQLRQSRQLMCYDIAHTLTGSVVSAIILWKGAGEVLNGTFTVGMLVAYLSYQMRFSSSISSLTDKYFSWRMLDIYNERLADIVLTPTEDYQQQPAQEDNDTSVFPSVFRGDVADGARVPLSLEHITFSHKGGNKPILRGVSLTLHPGEVMAITGQSGCGKSTLVKLILGIHIPDEGTIRAFGIPHTHPDYFQVRQRIGTVLQDDHLFRGSIADNIIFFSGDRNHERMIQCARLALIDSDIMSMPMGYQTLIGETGGGLSGGQKQRILLARALYKKPGFLLLDEATSHLDVESEIQISQTLRQLGLPVLLIAHRPETIASADRVLYLADGCFMELRHKRTIDDGLNKN

SEQ ID NO: 170 - protein, 735 aa, Enterobacter sp.
MDTLKWTGRKQLPLTRQTESAECGLACLVMMACWHGLQTDLPTLRERFSISSQGMTLQRLTECAAGIGLSSRAVRLELEDLRSLSLPCILHWNMNHFVVLHKVRGSRLVIHDPEKGKVTLSLQDAGKHFTGVALELMPASDFTARDERKKIHLRQLTGKTPGLLSAMSRIIVFAVALEILTLGSPLLNQLVIDEVLVAADQSLLTVIIIALLLMSLTQMLLSLARQWASITLSVNFNMQWTARVFHHLVHLPLAWFDARSKGSINARFEAVDTIQQALTTQVLEGILDVLLVVTALCMMLLYSPEMTLIAVLAAIFYGVLRALWYPSLRQSAEDAWDAGAKESGHFLETLNGILSLRINGVTAHREAAWLNLNVARRNTQLRQSRLLMCYDIAHTLTGSVVSAIILWKGAGEVLNGTFTVGMLVAYLSYQTRFSSSISSLTDKFFSWRMLDVYNERLADIVLTPTEGLQQQPFREHNNASHIPSVSRREMDEYAHAPLVLEHIIFSHKGGENPIIGGVSLTLNPGEVVAITGKSGCGKTTLVKLILGIHIPDEGRIRAFGILHTHPDYFQVRQRIGTVLQDDHLFKGSVADNIIFFSDDRNYERMIQCARLALIDSDVMAMPMGYQTLIGETGGGLSGGQKQRILLARALYKKPGFLLLDEATSHLDVESEIRISQTLRQLGLPVLLIAHRPETIASADRVLYLADGYLTELNLQHTVNNGESVCEYTNDVMAKN

SEQ ID NO: 171 - protein, 727 aa, Klebsiella variicola
MELMNWTGKKRLPLIRQTESAECGLACLAMMACWHGLQTDLTTLRERFSISTQGMTLQRLIECAAGIQLSSRAVRLEPEDLKSLSLPCILHWNMNHFVVLHKVRRSRLVIHDPDKGKITLSLQDAGKHFTGVALELMPSSDFTVRDERKKIHLRQLTGKTPGLLAAMSRIITFALSLEILTLGSPLLNQLVIDEVLVAADRSLLTVVIVALLLLSLTQMLLSLACQWASITLSVNFNMQWTARVFHHLVRLPLAWFDARSRGSVNARFDAIDTIQQALTTQVLEGILDVLLVVTALCMMLLYSPEMTLIAVLAAVIYGVMRTLWYPSLRQSAEDAWDAGARESGYFLETLSGILSLRINGVTTHREAAWLNLNVTRRNTQLRQSRLLMYYDIAHTLTGSVVSAIILWKGAGEVLNGTFTVGMLVAYLSYQMRFSSSISSLTDKFFAWRMLDVYNERLADIVLTPTEGNQQQTAQENNSTSTTPSIFQERVTEDTDLPLTLTHIAFSHKGSNKPILRGVSLTLHTGEVVAITGKSGCGKSTLVKLILGIHLPNEGTIRTFGIPHTHPDYYQIRRRIGTVLQEDHLFRGSIADNIIFFSEDRNHERMIQCARLALIDSDIMTMPMGYQTLIGETGGGLSGGQKQRILLARALYKKPGFLLLDEATSHLDVESEIQISQTLRQLKVPVLLIAHRPETIASADRVLYLTDGYFTDLTHQYRNGSEYLVRMD

SEQ ID NO: 172 - protein, 711 aa, Serratia inhibens
MDVTEMINWGWRKRLPLIRQTESAECGVACLAMIAGWHGHKVDMRTLRTECHVSQLGMTFSQLMTCAEGLNLSGRVIRLELDELHLLTVPCILYWDMNHFTVLRRVRGQTLELHDPARGQLKMSLQEANRHFTGIAMELTPNHRFETQDRREKIRLTTLIGKTHGLKPALARIFCFAVALEVLALLGPLINQLVIDEVLVALDASLLTLIVIAMLLMTGTQMLLELARQWATLTMAVNFNMQWTANVFHHLLRLPIGWFEARNMGDISAKFDAVDTIQDTLTTTLLEAFLDVLLVVGTLIMMFFYSVKLTLVALAAAAVYALLRLCWFSTLRKAAQDSWNAGTEESSHFLESLRGVLSLRVNGALMPRESAWRNLNVARRNAQLRESKLMMVYGIMQTVIGSVVAAAVLWLGAGSVLAGEFSVGMLVAYMSFQGRFSGSINGLIDKVVAYRMLDVYNERLADIVLTPREGAAASQAGNQGPVCPVSAWPADLPVLSLEQVSFKYAGTEREILSQVSLDIMPGEVVALVGASGSGKTTLAKLVLGLYPPSAGVIRTLGVEHRRADYQQTRQHIGAVLQEDQLFRGSIADNITFFSPKRDNDKLGECVRLAQLDQDIEQLPMGYQTLISEMGGTLSSGQKQRLLLARALYKEPKLLILDEATSHLDVTNEMLVSRTLRRLGLPILLIAHRPETIASADRVIELSAGRIRTQSL

SEQ ID NO: 173 - protein, 711 aa, Serratia liquefaciens
MDVTEGLNWGWRKRLPLIRQTESAECGVACLAMIAGWYGHRVDLPTLRAQFKVSQLGMTFSQLIACAERLHLSGRAVQLELEELHLLSLPCILYWDMNHFTVLKRVRGQTLELHDPARGLVKMSLQEANRHFTGVAMELTPTHQFVVKDQIKKIRLRELIGKTQGLKVALARIFCFALALEVFALIGPLINQLVIDEVLVALDASLLTLIVIAMLLMAATQTLLELARQWATLTMAVNFNMQWTANVFHHLLRLPINWFESRNMGDISAKFDAVDTIQDTLTTTLLEAFLDVLLVLGTLTMMFFYSVKLTLVALAAAMVYGLLRLFWFSTLRKAADDSWVAGTQESSHFLESLRGVLSLRVNGALTPRETVWRNLNVARRNAELRESKLMMVYGIMQTVIGSLVGAAVLWLGAGAVLAGQFSVGMLVAYMSFQGRFSASISGLIDKAVAYKMLDVYNQRLADIVLTAREYAAAQAAGNSTLLLSASAWPQDRPALSLEQVSFHYAGTESEILSQVSLDIMPGEVVALVGASGSGKTTLAKLVLGLYSPTAGVIRTLGIEHRQGDYSQVRQHIGVVLQEDQLFRGSIAENLSFFTPRVDQEKLVECARLAQLDQDIDNLPMGYQTLIGEMGGTLSAGQKQRLLLARALYKKPRLLILDEATSHLDVNNEMLISHTLRQLGLPILLIAHRPETIASADRIVELAAGRLHRRKG

SEQ ID NO: 174 - protein, 711 aa, Serratia liquefaciens
MDVTEGLNWGWRKRLPLIRQTESAECGVACLAMIAGWYGHRIDLPTLRAQFKVSQLGMTFSQLIACAERLHLSGRAVQLELEELHLLSLPCILYWDMNHFTVLKRVRGQTLELHDPARGLVKMSLQEANRHFTGVAMELTPTHQFVVKDQIKKIRLRELIGKTQGLKAALARIFCFALALEVFALIGPLINQLVIDEVLVALDASLLTLIVIAMLLMAATQTLLELARQWATLTMAVNFNMQWTANVFHHLLRLPINWFESRNMGDISAKFDAVDTIQDTLTTTLLEAFLDVLLVLGTLTMMFFYSVKLTLVALAAAMVYGLLRLFWFSTLRKAADDSWVAGTQESSHFLESLRGVLSLRVNGALTPRETVWRNLNVARRNAELRESKLMMIYGIMQTVIGSLVGAAVLWLGAGAVLAGQFSVGMLVAYMSFQGRFSASISGLIDKAVAYKMLDVYNQRLADIVLTAREDAAAQAAGNATLLLSASAWPQDRPALSLEQVSFHYAGTESEILSQVSLDIMPGEVVALVGASGSGKTTLAKLVLGLYSPTAGVIRTLGIEHRQGDYSQVRQHIGVVLQEDQLFRGSIAENLSFFTSRVDQERLVECARLAQLDLDIDNLPMGYQTLIGEMGGTLSAGQKQRLLLARALYKKPRLLILDEATSHLDVNNEMLISHTLRQLGLPILLIAHRPETIASADRVVELAAGRLHRRKG

SEQ ID NO: 175 - protein, 711 aa, Serratia plymuthica
MDVTEMINWGWRKRLPLIRQTESAECGVACLAMIAGWHGHRVDMRTLRAECHVSQLGMTFSQLMACAEGLNLSGRVIRLELDELHLLAVPCILYWDMNHFTVLRRVRGKTLELHDPARGLLKMTLQEANRHFTGIAMELTPTHRFETRDRREKIRLTTLIGKTHGLKPALARIFCFAVALEVLALLGPLINQLVIDEVLVALDASLLTLIVIAMLLMTGIQMLLELARQWATLTMAVNFNMQWTANVFHHLLRLPIGWFEARNMGDISAKFDAVDTIQDTLTTTLLEAFLDVLLVVGTLSMMFFYSAKLTLVALAAAAVYGLLRLCWFSTLRKAAQDSWNAGTEESSHFLESLRGVLSLRVNGALMPRESAWRNLNVARRNAQLRESKLMMVYGIMQTVIGSLVAAAVLWLGAGSVLAGEFSVGMLVAYMSFQGRFSASINGLIDKAVAYRMLDVYNERLADIVLTPREGATALQAGNKGPAYPAGDWPPDLPVLSLEQVSFKYAGTEREILSQVSLDIMPGEVVALVGASGSGKTTLAKLVLGLYPLSGGVIRTLGLEHRRTDYQQIRQHIGAVLQEDQLFRGSIADNITFFSPKMDSEKLGECARLAQLDRDIEQLPMGYQTLISEMGGTLSSGQKQRLLLARALYKEPKLLILDEATSHLDVANEMLVSRTLRQLGLPILLIAHRPETIASADRVIELTAGRLRTLGL

SEQ ID NO: 176 - protein, 711 aa, Vibrio alginolyticus
MRVVKDVYRKWEFGKKLSLVRQTEKAECGVACLAMIADWYGYKIDLRDLRAKFGITHHGMSFKRLIECGESLNLHAQAKGGVELHHLKELATPCILHWDSNHFVVLKAVKKDCVIVHDPARGECVFSFTELNKHFTGVVLELTPTDDFKQRDESKHIKLSRLIGKTHGLKRALGKILVLAIILEGLALLLPIMNQVVIDEVLVGYDEDLLVLVILGILLITAAQLVVSIVKEWATALMSVDFNMQWLANVFHHLFRLPIDWFEKREIGDIAAKFDAVGTIQHTLTTSVIQAFLDLILVLGTLSVMMFYSPLLSCIAMFAAILYIILRMVWYGAFKRAEENTWETSTKENSYFLETINGLLSLRVNGALNWRENIWRNLNIDRRNAQLHEMKLGMVYSVVSTTINSLVSAAVLWFGANLVISGDFSIGMLVAYLSYQGRFSGSVSSLIDKFFEYKMLSVYNERLADIVLTEREFDSISVDKVECFTQVVGQDDDILQFQNVSYSYAPDMPLILNSASFDIKGNEVVALVGESGCGKSTVSKLILKLYQPTTGNIIWFGNKGINPIEIRSRVGVVLQDDSLFGGSIIDNITLSAEAIDEEWMYECAQRARVHEDISRLNMGYHTLLGEQGGGLSGGQKQRILIARALYKKPNLLILDEATSHLDIDTERFVCSELRSLNIPTLMIAHRPDTIAAADRVLLLQDGLITELVRNQ

SEQ ID NO: 177 - protein, 716 aa, Vibrio casei
MHAEGLIDWGWGKKLPLIRQTEKAECGIACLAMVADWHGYKTDLRSLRVKCGITQHGMSFARLIECGALLKLSGRAVRLDLHELQQLATPCILHWNLNHFVVLKKVTGKKIEIHDPANGALALSHDEVNKHFTGIALELTPTHDFEEKVESKAIRLSTLIGKTVGLKGALGRIFVFALVLEVLALTLPIFNQIVIDEVLVGYDKNLLVLVIGAILMVTATQTLIGLAQQWATIKLSVNFNMQWAANVFHHLFRLPIDWFEKRDIGSISAKFLAVDVIQQTLTTSVIQALLDLVLVVGTLMVMLVYSPQLSLIAIAAALGYIALRLAWFGAFKRAEENTWEASTQEESYFIETVRGVLSLRVNGTLPWRESSWRNLNINRRNAQLHEQKLGMIYSTMNVAIVSLVSASVLWFGANLVLDGLFTIGMLMAFLSYQGRFSASISSLTDKYFEFKMLSVYNERLADIVLTEKETDSDSNVIGQTNVTNNPNDNVIEFVDVYFSYGKDQSPILNGASFSVKSNEIVALVGPSGCGKSTISKLLLGIYSATSGSIYYFGDKSMASKPLRTQVGAVLQEDQLFSGSIIENITFFAGDLDEEWLVECARRAGVHDDIERLNMGYHTLIGEMGSSLSGGQKQRVLIARALYKKPKFLVLDEATSSLDIYTESFVCQMFKEIKIPILMIAHRPETIAAADRVLLMDGGLVQEIPNDFRREVQDVSL

SEQ ID NO: 178 - protein, 698 aa, Escherichia coli
MTNRNFRQIINLLDLRWQRRVPVIHQTETAECGLACLAMICGHFGKNIDLIYLRRKFNLSARGATLAGINGIAEQLGMATRALSLELDELRVLKTPCILHWDFSHFVVLVSVKRNRYVLHDPARGIRYISREEMSRYFTGVALEVWPGSEFQSETLQTRISLRSLINSIYGIKRTLAKIFCLSVVIEAINLLMPVGTQLVMDHAIPAGDRGLLTLISAALMFFILLKAATSTLRAWSSLVMSTLINVQWQSGLFDHLLRLPLAFFERRKLGDIQSRFDSLDTLRATFTTSVIGFIMDSIMVVGVCVMMLLYGGYLTWIVLCFTTIYIFIRLVTYGNYRQISEECLVREARAASYFMETLYGIATVKIQGMVGIRGAHWLNMKIDAINSGIKLTRMDLLFGGINTFVTACDQIVILWLGAGLVIDNQMTIGMFVAFSSFRGQFSERVASLTSFLLQLRIMSLHNERIADIALHEKEEKKPEIEIVADMGPISLETNGLSYRYDSQSAPIFSALSLSVAPGESVAITGASGAGKTTLMKVLCGLFEPDSGRVLINGIDIRQIGINNYHRMIACVMQDDRLFSGSIRENICGFAEEMDEEWMVECARASHIHDVIMNMPMGYETLIGELGEGLSGGQKQRIFIARALYRKPGILFMDEATSALDSESEHFVNVAIKNMNITRVIIAHRETTLRTVDRVISI

SEQ ID NO: 179 - protein, 704 aa, Enterobacteriaceae sp.
MQQDRSMTNRNFRQIINLLDLRWQRRVPVIHQTETAECGLACLAMICGHFGKNIDLIYLRRKFNLSARGATLAGINGIAEQLGMATRALSLELDELRVLKTPCILHWDFSHFVVLVSVKRNRYVLHDPARGIRYISREEMSRYFTGVALEVWPGSEFQSETLQTRISLRSLINSIYGIKRTLAKIFCLSVVIEAINLLMPVGTQLVMDHAIPAGDRGLLTLISAALMFFILLKAATSTLRAWSSLVMSTLINVQWQSGLFDHLLRLPLAFFERRKLGDIQSRFDSLDTLRATFTTSVIGFIMDSIMVVGVCVMMLLYGGYLTWIVLCFTTIYIFIRLVTYGNYRQISEECLVREARAASYFMETLYGIATVKIQGMVGIRGAHWLNMKIDAINSGIKLTRMDLLFGGINTFVTACDQIVILWLGAGLVIDNQMTIGMFVAFSSFRGQFSERVASLTSFLLQLRIMSLHNERIADIALHEKEEKKPEIEIVADMGPISLETNGLSYRYDSQSAPIFSALSLSVAPGESVAITGASGAGKTTLMKVLCGLFEPDSGRVLINGIDIRQIGINNYHRMIACVMQDDRLFSGSIRENICGFAEEMDEEWMVECARASHIHDVIMNMPMGYETLIGELGEGLSGGQKQRIFIARALYRKPGILFMDEATSALDSESEHFVNVAIKNMNITRVIIAHRETTLRTVDRVISI

SEQ ID NO: 180 - protein, 703 aa, Vibrio cholerae
MNSSEFAINVSKLLEFGRIKRVPTILQSEVDECGISCLAMVSSYYGNKINLPPLRRDSNFSNEGMKLIEVMNVANSLNLTSRALQCSLDDTGKLKLPCILHWELNHYVVLTDIIKGNYYINDPAIGRRILTTKQFSDSFTGIALELTPNSEFKTSDSRVVMGIGQLWNKLLGIKRSLFSLFILTIILQFFALLSPYYMQWVIDHVLLSHDMPLLAILALGFSLLKVIQIIVSSFRTWLIIRISSSLNIQMGANLFNHLLRLPISFFEKRHVGDIISRFGSLNIIKQMISTGFVEGLIDGIMAIIVLIMMYIYSPILANLVVVIIFIYFLIQCFFYNPTRTIEEELINSEAKEDSIFLESVRGIQSIKLFTLEATRLNTWLNKYADVININIRKSKIGICQNILTSLLFGLESILIVYIGAVTVMDGKLTVGMLLAFIAYKSHFISSISSFIGKCIAFKMLSLHLERLSDIALETEETKISSINSDFFGKSNGFLRVENLGFRYSEKSDWVFNGVSFEISPGECLAITGVSGCGKTTLMKVILGLLKPTEGKIYLDGVESSQWPLDLYRKKFSAVMQKDTLFSGTLIENITMFEQDYDENRLVDCCRMACILDDIRALPMGFHSLVGDMGSSFSGGQLQRVYLARALYRNPLILCLDESTSHLDMSNESLINENLKDTFFTKIIIAHREETISTADKIYRLDKI

SEQ ID NO: 181 - protein, 713 aa, Agarilytica rhodophyticola
MYFFGRLENSIKKKLPLIMQNEASECLLACIAMILNYHGRKVDLIELRDSINISSRGSGFSHLLNISNKLNIKTRPVKVDIKSINKLSLPCILHWKKNHFVVLKSINKSGIYIHDPSIGKRFVIYKNLSEFFFGIAAEVTPTENFKKKDKKENKDIKKFFKDFSGIKRNILYIILLSFFLQLISLSTPFYIQTIIDKVIQSHNIKLLEILFLCFIFLIIIQSIISYSRSYITNLFSFKYGFHSSSRLFYHLIRLPTLYFSRRGLGDVITKFEAQGYLKNYLSNEVVSSFIDAIMLIIAIIAMLFYSVKLSIIVFAVSVFYVVLDFNNFKIQKVLLEDAVSFGAKGHTAFIESVRLIESVKILENEKNRHYKWLTYFSQETDKRYESAHLENKIEFLRQIIFGLENITIIYLGSLMVIKNSISLGMLIAFISFKVKYVDTVKSLMRQIMSLNTLNVYMNRLSDIVRAKTDINTSGSKSYFGDDNDDKTSCISKFQRRNEYIISGKIEVINLAFRYGDSENFVFRNINFTIKQGEKVVIVGGSGCGKTTLLKCLMGLYLPTEGTILIDGKPLAEVGNYRSQISGVLQNDSLFSGTIAENISGFDPLPDLVKIRNCAKLSSINIDIDSMPAGYNTYLGDMGGGLSGGQMQRIIIARALYSNPSILFMDEATSHLDVGNENIVNTNLNNLNITQVLVAHRMETVMSAERQINISNFN

SEQ ID NO: 182 - protein, 755 aa, Alteromonas macleodii
MMNITHPLSTQILYTTKRVPVVLQSEAAECGLACMTMIAQYYSDKRDLNALRQTVSVSLRGTTLKDVMRIASDLGFQTRAIKIEMEHLAQLSCPAILHWNMNHFVVLTKVKGKTLTIHDPALGERKLTYSEASKYITGIALEVTPSDTFSPKKSAPRLGLSQFFTRTTGFKRNLLTLFALSIVLQLFALASPYYMQTVVDDVLIYNNDALLKALAIGFALLLIIETFTSGIRKFVILSVSSRLQLQMSASVFKHLLSLPLDYFDKRHIGDVVSRFGSLASIREFLTTGLVTALLDGLMAVITLAVMILYSPKLTLVVVAIMLAYLAVRLGLLPFIKRLTTERIALAALEQSHFMESVRAILPIRVYSQEVQRHGQWQNKLVATLNKDITLGKINIGSALTNQLLFGAENLVVVYIGASLVMEGTLSIGMLLAFMAYKARFVSALDGVVNKLIELSMLGVHFNRLSDILLTPSQHKPLSLKAKDDTFEKQNVHFSNADVPNSSTSNSLAPNVLTPDGASTPSLLDAKTRPVALKATALSYRYSETNAWVFKNLMFSVHSGEIVAITGDSGCGKSTMLKCLMGLYPVSEGNIEHPSSTNPVIASVLQEDTCLSGTIAQNICCFEEAPDLKKMVYVAQLACIHEDIMQMPMQYHSLVGDMGSSLSGGQKQRLLLARALYQEPDILFLDEASSHLDMENEAQINHHLKSLNMTRIIVAHRPQSIAMADKVYRLENGNLHLCPPTEVTGSATSGKQGETL

SEQ ID NO: 183 - protein, 733 aa, Alteromonas pelagimontana
MTAIPLNLPPLSLSIIRRRAVPMVFQSEAAECGLACLCMIARYFKDGSDMTELRQKYGVSLRGATLKSIMDMAADMGLSARALKLETEHVKQLTLPCILHWDLQHFVVLTKVSQRGVVIHDPALGERALNWDDVGNHFTGIALEITPTSEFKPARQAPTLTLRHFWQRIVGLKRHLLILFALSALIQLFAIASPYYMQTVIDDVLLRQQRDLLLVLALGFGMLLLIESATSVLRRMAILALSSRLHQQMSANVFHHLIRLPVSYFSKRHMGDVVSRFGSLSQVRELLTVGVVTAVLDGLLALVTLAVMMVYSVTLSGIVAVVVLIYLAARIALISTMKRLNAERISIAAKEQSHFMESIRAIQTVKQFGQEMNRQSQWQNKLADTINTDIRLGKWDISVSCLNQLLFGFENILIIYFAATEVMDNAMTVGMLYAFIAYKTRFITAIDGLINKAIEFKMVAVHFERLADIVFTKQESFLQTAPSLPQLSDNIIDAPTRPLVIKDLCYRYSPADAPVFENINLTIEAGSTVAIVGPSGAGKTTLLKCLMGLLPPSQGDVLYGGDSVFSAGVFRQLTSSVLQDDNCLSGSIMDNITCFTDSPDFERMVQAALKACIHEEIMAMPMQYQTLVGDMGSTLSGGQLQRLLLARALYRQPKILFLDEASSHLDLNNEAAINQHLKALAITRVVVAHRSETIAMADRIYLLRNGTLCEVPHSAVLSSPSPTSKNPPGENYD

SEQ ID NO: 184 - protein, 697 aa, Photorhabdus sp.
MMKLEKIIFWHSHNLPSILQTEIAECGLACLASISSYHGYQVDLSSLRKKFRIPLTGTNLNDIAYYAKELKLSYRAVKLDINEINQLKLPCILHWELNHFVVLKKISKNTITIMDPSIGFRRLSYNEFSNCFTGIAVEFWPNNDFKKKIDKEKVDILSFFKNISGIKRSLVKILLLAFVLEFFALIGPFYMQLIIDHGIVSEDKNLILLLSIGFGVLLVFEQVINIIQSLLTTYISTNLNIQWKANVFTHLMNLPVSFFEKRHLGDIISKFSSIDSIQNTLTSSFFISICNSLVSATTIIIMTMYNFQLTLISIVTIFIYIIIRIIWYYPLKEATKENIVQSAKLSSNFMETMRGIRAIKIFCKENHRYNSWLTLSINTVNSALKSQRLSLGFSFSNRIIFGIQNILIVYIGTSFIFDNTFTVGILISFLAYKTQFESRTISLIDLFISFKMLGIHIERLSDIVLSETELKSNILLKTETIKSNKIEIKNLSFKYEGNNNYLIENLYFDIQPGDSVAIIGPSGCGKSTLLNLMIGNILPTGGEVRIGGVIVNKTHPKIIRDNIGYVAQDDSLFSGSIMENITFFDEHPDTDKAINCAKIAAIHHDIKKMPMEYETLIGDMGTILSGGQKQRLCLARALYKDPKILFLDEATSHLDTENEKIISSNLKMLSITKVMVAHRKETIESANKLIDLTKKCA

SEQ ID NO: 185 - protein, 725 aa, Shewanella oneidensis
MTIATDKHAALMASESNSPVDLLEFSGNKRVPLILQAEMAECGLACMAMIASFNGHKLDMAALRKRFTANLKGMNLQQLISLGDSIGLSSRALKCPLEEVGKLALPCILHWDMNHFVVLTGVTKKSISINDPAAGKRTLSLQEFAKHFTGIALELTPTKAFVKQDERQQMRLSQLWTKISGVNAALITLLLLSVLLQVFALVTPYYMQWVVDEVLVSQDQPLLIVLAIGFGLLVVINVFTTGVRSWLVLRVSSLLNMQMGVNLLRHLLRLPMNYFEKRHIGDLVSRFGSLAQVRERLTTGLVETVVDGVMSIAVLVMMLIYSVKLTLVVMAAVALYTLMRFALYRPLHRATEESIQAKAKEQSNFLENIRGIQTIKLFTCESARQGIWQNRYSEVINADIRLGRLKISFDAMNKLLFGVENIIVIYMAAMIVMSGGLTIGMVLAFIAYKNQMTERVASLIEQLIMFRMLRLHLDRISDIALHEQEAHQEGFTPLNVVKGRLSLENVSFRYGENEPEVVSNLSLDIQAGESVAIVGASGCGKTTLVKLMLGLLVPSSGRILLDGQAIQQIGLTQYRQQIAAVMQDDTLLSGSIADNITFFDPEPNYVKMQQCAQLAVIDMDIAHMPMGYNSLVGDMGNQFSGGQVQRLLLARALYQSPSILFMDEATSHLDIMNEAKISEQIKNLNMTRIIIAHRPETIKQADRVVVMHQGKIMTAEELQQAQSAS

SEQ ID NO: 186 - protein, 725 aa, Shewanella putrefaciens
MTIAAEKQAALAINEHNSPVDLLEFSGKKRVPLILQAEMAECGLACMAMIATFNGHKLDMAALRKRFTANLKGMNLQQLISLGDSIGLSSRALKCPLQDVGKLALPCVLHWDMNHFVVLTGVTKKSISVNDPAVGKRTLSLQEFAKHFTGIALELTPTKAFVKQDERQQMHLSQLWTKISGVNAALITLLLLSVLLQVFALVTPYYMQWVVDEVLVSQDQPLLIVLAIGFGLLVLINVFTTSVRSWLVLRVSSLLNMQMGVNLLRHLLRLPMSYFEKRHIGDLVSRFGSLAQVRERLTTGLVETVVDGVMSIAVLVMMLIYSIKLTLVVMAAVALYTLMRFALYRPLHRATEESIQAQAKEQSNFLENIRGIQTIKLFTCESARQGIWQNRYSEVINADIRLGRLKISFDAMNKLLFGVENIVVIYLAATIVMSGGLTIGMVLAFIAYKNQMTERVASLIEQLIMFRMLRLHLDRISDIALHEQEAHQESFTQLNIVKGRLSLENVSFSYGMNEPEVISNLSLDIQAGESVAIVGASGCGKTTLVKLMLGLLVPTDGRILLDGQAIQQIGLTQYRQQIAAVMQDDTLLSGSVADNITFFDPEPNYVKMQQCAQLAAIDMDIAHMPMGYNSLVGDMGNQFSGGQVQRLLLARALYQSPSILFMDEATSHLDIMNEAKISEQIKNLDMTRIIIAHRPETIKQADRVVVMHQGKIMTAEELQQAQSAS

SEQ ID NO: 187 - protein, 709 aa, Vibrio harveyi
MANSNVGHSSSSNFNAKTLLNFNGVKKVPLVLQSENTECGLACIAMISSFYGHKINLLPLRHHTNLGDQGVELLRLMDIANDLSLVPRALQCSLEEVTHLALPCILHWNLDHYVVLTKIKKGRYYINDPAGGEVIVNKKQFSDAFTGIALELTPNRNFKKEDSRVLMKIEQLWGRIIGIKRSLIALFSLSILIQILALLSPYYMQWIIDNVLLSHDKPLLLVLAIGFSFLKIIQVFISSFRSWFMIRLNCEISIQLASNIFDHLIRLPISFFEKRHVGDIVSRFGSFNAIKEMITTGLVEAIIDGLMAVIILFMMYIYSPILSSFVLVFISISLIVQYAFFYPSKRLEEESIVFEAKEDTMFLESVRSIQTLKLFGQETNRLNGWLNKYADVTNIGIRKSRLNLTENIITTLLNGFESILILYLGALIVIDGELTIGMLLAFIAYKSQFTSNISALVGKWLSFKMLSLHLERLSDITLEDKEKLNFSSEQIKTSGSTIRVEGLGFRYSESSEWIFRDVSFEVESGSSIAIVGPSGCGKSTLMKVILGLLEPTEGKIFFNDIDISQLSLSNYRQQFSAVMQNDSLLSGTIAENITMFDLKFEQSKLEECCKKACIFDEIINLPMRFNSLVGDMGSVFSGGQLQRIYLARALYRDPKVLCLDESSSHLDKANEKKVNQSLRELDITKIIIAHSAETINMADRVINLDGYSH

SEQ ID NO: 188 - protein, 688 aa, Pantoea agglomerans
MPFFHQAESSECGLACLAMISSYYGLHKDLFSLRSRYMTPASGARLADVIQIAEDSGMLTRALSLEVSELKLLKLPCILHWDFNHFIVLVKTTKKDFIVHDPASGRLKLTRKEIEENFTGVALEIFPCSDYQERADDKAERIEITKLTKNIKHLTSNLLKIFFLSVVIEAIILLIPIGTQLTMDYVLPSADQSLLTLICTGLFIFILLRSLLSSVRAWLTLKIGSTIDVQWQMNLYKHLINIPLSYFERRKTGDVQSRFSSIETIRSTIVSGITGAIIDTIMCMILLLLMFLYSGLLSVIATGITAVYVIIRIVSYRFYKRLQEESLVKSARASSHFMETLYGISAVKAHGLKNGRVSQWINLKIDSINANLQISKVDMVYSGVNTFVSAVDQVTLLWVGANLVIHNEISLGMFIAFGTFRSQFSDRIASLTGYAFNYKLLDLHSERLSDIAMQKQEGETAHDAIIRHTEPSDPAELVVECISYSYNQFSRPVLKDISFTVEPGESVAIVGSSGSGKTTLMKLISGLIRPEKGRILIDRTDIHKSSSGKVNARLGCILQDDKLFSGSIRENITCFSEVPDEEWMIDCARKALIHDVIMTFPMGYDTLLGEVGEGLSGGQKQRVYIARALYKRPNILVMDEATSALDNVSEKLVSNSINSLRITKVIIAHRESTIAGADRIIRLDELNT

SEQ ID NO: 189 - protein, 699 aa, Pantoea allii
MNEELHTILRGLLQFSGRGRVPLHLQTETSECGLACLAMVAGFHGSHTDLLTLRERSGLSSRGSTLTSLISVAGEMNLAARALLLDLDSLSELRKPCILHWDLNHFVVLVSVKGKRCIVHDPAAGRQSMSLEDLSSHFTGVALELWPDSDFKPDIRQKKLKVSTLLNSITGFQQSLVKIFCLSLMIEFITLLMPVATQMVMDNAVPAADTGLLTLICLSLLLLTLLQACLGLFRGWTMMVMGIYTDLQWKDGLYRHLLRLPLSWFEKRRIGDIQSRFASLDTLRNTFTQSINGAVIDGVMATGALILLILYGGWLAWVVTGFTLIFVLLRVFTWPRYRQAQEELLIKNARAASSFTETLYAAATVRAQGLSHRRRETWLNMMADATGSGVSLLRFDMLAGIAGTFIAASDSVIILWLGISAVINHTMTLGAFVAFSAFRGMFSERILSLTGVALQLRMLSLHNERIADIALSEPEGDKEDEKRLFPEGEALALKCEALMFRYDRFSPPVLQNLNISIGAGESVAITGASGRGKTTLMKILCGLTTPVKGRVLVNNVDIKAAGLGNYRRSVACILQDDRLLAGTLRDNITGFSHDVDEDWMVDCARLSHIHDDIMALPMGYETLTGELGEGLSGGQRQRLFIARALYRRPGIIFMDEATSHLDEKNEALINAAMQTLKMTRVLIAHRPSTISSADRIIEL

SEQ ID NO: 190 - protein, 698 aa, Escherichia coli
MTNGNFRQIINQLDMRWRRRVPVIHQTETAECGLACLAMICGHFGKNIDLISLRRKFNLSARGANLAGINGIAEQLGMVTRALSLELDELGALKMPCILHWDFSHFVVLVSVKRNRYVLHDPARGRRYLGREEMSRYFTGIALEVWPGSEFQAETQQTRISLRSLINSIYGIKRTLAKIFCLSVVIEAINLVMPVGTQLVMDHAIPAGDRGLLTLISAGLMFFILLRAAVSMLRAWSSLVMSTLINVQWQSGLFDHLLRLPLAFFERRKLGDIQSRFDSLDTLRATFTTSVIGLIMDSIMVVGVFVMMLLYGGYLTWIVLCFTTIYIFIRLVTYGNYRQISEECLVREARAASYFMETLYGIATVKIQGMVGIRGAYWLNMKIDAINSGIKLTRMDLLFGGINTFVTACDQVVILWLGAGLVIDNQMTIGMFVAFSSFRGQFSERVTSLTSFLLQLKIMSLHNERIADIALHEKEEKKHEIEIVAHMGPISLETNDLSYRYDSQSAPIFSALSLSVAPGESVAITGASGAGKTTLMKVLCGLFEPDSGRVLINGIDIRQMGINNYHRMIACVMQDDRLFSGSIRENICGFAEEMDEEWMVECARASHIHDVIMNMPMGYETLIGELGEGLSGGQKQRIFIARALYRKPGILFMDEATSALDSESEHFVNVAIKNMNITRVIIAHRETTLKTVDRVISI

SEQ ID NO: 191 - protein, 413 aa, Klebsiella pneumoniae
MKWRGRAILLPGIPLWLTMLGSIAFITAFLTFVIAGTYSRRVNVSGEVTTWPRAVNIYSGVQGFVVRQFVHEGQSIKKGDPIYQIDVSKSTRSGVVTDNQRRDIENQLVRVDNIISRLEESKKITLNTLEKQHLQYSDAFRRSSDIIRRAEEGIKIMKNNMENYRNYQLKGLINKDQLTNQVALYYQQQNNLLSLSGQNEQNALQITSLESQIQTQAADFDNRIYQMELQRYELQKELIDTDVGGEIIIRALSDGKVDSLSVTVGQMVNAGDSLLQVIPENIENYYLILWVPNDAVPYISAGDKVNIRYEAFPAEKFGQFSATVKTISRTPASTQEMLTYKGAPQNTLGTSVPWYKVIVKPEKQIISYGGKSLPLENGMKAESTLFLEKRRIYQWMLSPFYDMKHSATGPIDE

SEQ ID NO: 192 - protein, 424 aa, Chania multitudinisentens
MFRPEAILSHRMQWRGQALLLPGIPFWAMAGSCLFFIATFLTFIIAGTYTRRVNVVGEISTYPRAANVYSSVQGVVTKQFVTEGQMIKAEEPIYQIDVSKSTRSGVISDNQRRDIGNQLTRIAQIIIRLENSKKATLNMLEAQKAQYSTAFQHSTDILQRAQEGIRIMKENMENYRRYQSKGLINKDQLTNQIASYYQLQNNLLGLSSQNEQNALQINALGSQIQTQAAEFDNQIYRMEMQRYELQKELLNIDADGEIIVRALADGRVDSLSVTVGQMVNVGDSLLQLIPHNIDHYSLVLWVPNDAIPYVSIGNRVNIRYEAFPAEKFGQFVGTVAVISKTPASPQEMLTYQGAPRAAQTTAVPYYKVIVSPKKQTIVYNAQRLNLQNGMKAQSTLFLEKRKIYQWMLSPFYDMKHSVMGPVNE

SEQ ID NO: 193 - protein, 428 aa, Klebsiella sp.
MLKNIYRKEAIEYKKIHWKGKALLLAGIPAWLVTLLASLFLIALVLSLIFCTFTQRIDVRGEVITLPHSVNVFAPQQGFVVNQHVKVGDIVNKGQPLYELDVSRNTINGNVSAAQIEVINEKIANAEDIISKLMRNKSETLTALEKQIKTTSASLAETNRMLATTQVGLNKMFTNLSSYDKYLKDGLITKDQYNYQHSLYFQQQSAYQSLVTQKMQLESQLTQTNSDKITKAADFDNQISSQHNQINDYKNQLVESNANGNLIIKATTNGKIESLAVTKGQMVENGSSLAQIKPTGDIEYYLILWLPNNTIPYVKPGDTINIRYDAFPADKFGQFPGKVISISSVPASRQEMAEYTNVNNGTSQQELALYKAIIKIKDKTFSYNGKTLTLSNGLKAQAVVFLEERPLYMWMFTPFYKMTQSVSGPIDE

SEQ ID NO: 194 - protein, 416 aa, Klebsiella pneumoniae
MTCLFAPSKALLLAGLPAWLVTCLSLLFLCALICALIFCKFTQRIDVKGEVITLPHSVNVFSPQQGFVVNQYVQIGDVVKKGQTLYELDVSRNTTTGNVSAAQIEVINEKIANSEAIIKKLTHNKNETLIALDAQLKNARNSLNETVRMLANTQQGLSKMHENLSSYDKYLKEGLITKDQYNYQHSLYFQQQSAYQSLISQKMQLETQLTQLSSDKVTKAADFDNQISSQYNQTNDYKNQLVESNANGNIIIKATTEGRIESLAVTKGQMVDKGSSLAQIKPIGNIEYYLILWLPNNSIPYVKVGDTINIRYDAFPSDKFGQFPGEIISISSLPASRQEMSEYTNVNDGTNQQELALYKAIVKIRDKKFNYDGKELSLSNGLKAQAVVFLEERPLYMWMFTPVYKISQSVSGPVND

SEQ ID NO: 195 - protein, 305 aa, Mannheimia haemolytica
MKSQIQATEQAITALYKNKSETINSLEKQIKNSQKIYQDKQSYLVEIEKSMNDYAELVKRYEKLLKVGHSSHDEVNIQKSRYFQQKSLFNELKQELIQLKSTQLSLENEIETRKTEFNNQIIRYEMQKSDLNIRLMEFESVSDIIVNASLDGKIESTSVTVGQIIKENDPLAQILPQNKGNYQLVMWVPNSAISFIKQGDEVNIRYEAFPFEKFGQFKGKITSISTLPASLQELSFYKNLPVNLEQGIPLYKILIELADQNVKYNDTSLYFMSGMKAEATLFLEKRKLYEWIFFPMYQLRKNMEQ

SEQ ID NO: 196 - protein, 376 aa, Mannheimia haemolytica
MIFGSYTRRETVFGELVMQAHPIILSAPKSGYISENYIQPHQQVKKGDPLFKITLDRITHSGNINVNSILSLKSQIQATEQAITALNKNKLETINSLENQIKNNQKIYQDKQAYLVEVEKTMNDYEDLVKRYEKLLKVGHSSYDEVNIQKSRYFQQKSLFNELKQELIQLKSTQLSLENEIETRKTEFNNQIIRYEMQKSDLNIRLMEFESVSDIIVNASLDGKIESTSVTVGQIIKENDPLAQILPQNKGNYQLVMWVPNSAISFIKQGDEVNIRYEAFPFEKFGQFKGKITSISTLPASLQELSFYKNLPANLEQGIPLYKILIELADQNVKYNDTSLYFMSGMKAEATLFLEKRKLYEWIFFPMYQLRKNMEQ

SEQ ID NO: 197 - protein, 424 aa, Salmonella enterica
MFRQEALDNRKMKWRGRAILLPGMPLWLIMLGSIAFITAFLTFVIAGTYSRRVNVSGEVTTWPRAVNIYSGVQGFIVRQFVHEGQSIKKGDPIYQIDVSKSTRSGVVTDNQRRDIENQLVRVDNIISRLAESKKITLNTLEKQRLQYSDAFRLSSDIIRQAAEGIKIMKNNMENYRNYQSKGLITQDQLTNQVALYYQQQNNLLSLSGQNEQNALQITSLESQIQTQAADFDNRIYQMELQRYELQKELVDTDVGGEIIIRALSDGKVDSLSVTVGQMVNAGDSLLQVIPENIENYYLIIWVPNDAVPYISAGDKVNIRYEAFPAEKFGQFSATVKTISRTPASTQEMLTYKGAPQNTLGISVPWYKVIVKPEKQIISYDGKSLPLENGMKAESTLFLEKRRIYQWMLSPFYDMKHSATGPIDE

SEQ ID NO: 198 - protein, 424 aa, Salmonella enterica
MFRQEALDNRKMKWRGRAILLPGMPLWLTMLGSIAFITAFLTFVIAGTYSRRVNVSGEVTTWPRAVNIYSGVQGFIVRQFVHEGQSIKKGDPIYLIDVSKSTRSGVVTDNQRRDIENQLVRVDNIISRLEESKKITLNTLEKQRLQYSDAFRLSSDIIRQAAEGIKIMKNNMENYRNYQSKGLITKDQLTNQVALYYQQQNNLLSLSGQNEQNALQITSLESQIQTQAADFDNRIYQMELQRYELQKELVDTDVGGEIIIRALSDGKVDSLSVTVGQMVNAGDSLLQVIPENIENYYLILWVPNDAVPYISAGDKVNIRYEAFPAEKFGQFTATVKTISRTPASTQEMLTYKGAPQNTLGISVPWYKVIVKPEKQIISYDGKSLPLENGMKAESTLFLEKRRIYQWMLSPFYDMKHSATGPIDE

SEQ ID NO: 199 - protein, 433 aa, Serratia inhibens
MEQDDLFRREAVSHNRSKWAGKALLIAGLPAWATAVISLSFIAVLVIFVTYGTYTRRISVGGEVTTLPRTVSIFATQQGFIAQRFVNVGEQVKKGQHLYQIDVSRATDSGTVSANTRQAIENQLAQVDSIISKLEQNKKTTSDNLRAQIEQYKLSHDQSKKMVENSRNGVNFMRETMKNYNEYRNKGLINKDQLNNQVYLYYQQQNTFQSLYSQSIQEALQITNLSSDLVTRASDFDNQISQYQFQKNDLLRQLSEADASGSLIVNAPTNGRVESLSVTPGQMVNVGDSLTQLIPSDHAIYYLVLWLPNSSVPYVNSGDNINIRYDAFPFEKFGQFPGVIDNVAYVPVSNQEMSSYGSSPMRQNGEVVESYYKVLVAIDNTRFGYQGKELNLSSGMKAQATLFLEKRPIYQWMFAPFYDMKNSVSGPIREQNL

SEQ ID NO: 200 - protein, 419 aa, Mixta gaviniae
MSESLFRQEALEANKTSAIGNVALYCPPYRWLVISLVAAIAIAVAAFFTFGSYTKRETAIGQLMPAKGVMNVASMAAGTVIDIHVEEGQRVEKGEAIATVSSEVFTALGQTRETVAQQLRLQRERLRGDLDNQQKLFAEAMSGLQERERLLGSQLEQLDLQQTQRARQARLARRQLDKLNLMRKEGYASNSQVEQQEAAVIDADARLQDIARQRLDIHQQLAQTRQQLRELPMNTRNKQNEIERRLSELEQSMAENESRRSIVLRAPMEGMVGSVMVKAGQMVSAGQTLFSVLPDEGRLQARIMVSSRAIGFIRPGQKVVLRYQAFPFQKFGQQYGKVAEVSRVALSPQEVATLTGNNNVQEQHYRVVVALEKQHINVYGRQEKLRPGSALEADFLIDTRRLYEWVLEPLYALGRRAGN

SEQ ID NO: 201 - protein, 421 aa, Acinetobacter sp.
MSSNLFRKEALDAKQAKWTGTIILSRPFSFTYLTICALCIALVIIAFAIWGSYTKRSTVQGQLIPQSGLIQVYTTQSGTILKKNVHEGQTVKKGDILFTISTASYGEQGSIADALAKQTQLKEQSIRNEITRMRFIHQDEKRTINNQIGLLNGTLVKVENLIANQRERVNLAKKNQQRYGNILHENAISHEEFEARKIDYLDQLAQYESLQREKVSLEKQLTEQQISLSGLENRQNNQIEQLERLLSSNTQELIEMQSRQHIAIQANSSGVIGTINAEVGQFVDLSKPLLTVLPENTALIAQLYVPSRAIGFVKEGDQVLLRYQAYPYQKFGHAKAQVLSVAKTALASQDLQTIGIISPQEQLNNEPVYLVRAKLDKQMVKAYGNDMPLQVGMTLEGDIMHEQRKLYEWVLEPLFSITGKL

SEQ ID NO: 202 - protein, 421 aa, Acinetobacter baumannii
MSSNLFRKEALDAKQAKWTGTIILSRPFSFTFLTICALCIALVIIAFAIWGSYTKRSTVQGQLIPQSGLIQVYTTQSGTILKKNVYEGQSVKTGDILFTISTESYGEQGSITDALAKQTQLKEQSIRNEISRIRFIHQDEKRTINNQISLLNGTLVKVENLIYNQLERINLAKRNQQRYEIMLKQDAASYEEFEARKINYLDQLAQYESLQREKVSLEKQLTEQQISLSGLENRQNNQIEQLERLLSSNTQELIEMQSRQHIAIQANSSGLIGTINAEVGQYVDLSKPLLTVLPENTPLIAQLYVPSRAIGFVKEGDQVLLRYQAYPYQKFGHAKAQVLSVAKTALASQDLQTIGIISPQEQLNNEPVYLVRAKLDKQIVKAYGKDMPLQVGMTLEGDIMHEKRKLYEWVLEPLFSITGKL

SEQ ID NO: 203 - protein, 417 aa, Enterobacter sp.
MSETLFRQQAIDNNRVKSLGSVILFTPPYRWLLVVFVGMIGAAIVTLLIFGSYTKRESARGTLVPEQGILDIVPVGSGTLTDIFIHEGQRVKKGEKLAAISSDISTAMGATHKQIAQQLNEQREVLKSELVNLDAINSSTMQGIHDRIGLLKEQIAQLQLINNQRRSQIGIEQEKLKNLRSLLAEGYASNTQIDQQESVRLEAVVRMQDVGRQLLELKQQLAQLEQQYIEQPINYLKQKNEVNQKIAELNQTMLENESRRGTILTAPADSIVGSVLVKKGQIVTSGQTIASLIPSDGNLQARVMISSRAIGFIKPGQKVVLRYDSFPYQKFGQQYGSVLEVSESALTPKDVASVTGDNLAQEQFYQVKIVLKKQSISAYGIEHRLPPGSAVNADFIVDKRHLYQWVLEPLYALGKRF

SEQ ID NO: 204 - protein, 340 aa, Limnobaculum parvum
MTDKKVKEGEFVKKGDELYVISSERESQNSHGTQQVISAQIQSRLASLNEDLTKNQTQFTSEKSLLQSYIRQLNDQVNTLDKQIVNQRKILQLVQRRQLQYQEIYKKEYISLEQFERVKEELLQQQSALSSYEREKINAQKELSGRQSELDGLLLKFDKQQAQIDRNISTTRQELAESEAKREITIQAPQTGTITAAVAQAGQFVDSSKPIVSIVPENSLLMVHLYAPSNAVGFIQEGAEVWLRYQAYPYQKFGQYRGKVVSISKAALLQHELQSTMNVGNEASLYQIVVKPDKQRISVYGKDKALQPGMELEADITLDKRRLYEWVLEPLLSITKKATD

SEQ ID NO: 205 - protein, 419 aa, Pantoea vagans
MEQSLFRQEALDAANRGNLGIVALYCPPYRWLVISVVVFITAVTALFFIFGSYTKYESSTGELLPENGMLIVPPPVSATVVDIPVKEGQLVKKDDVLMVLSSEVSTQMGQTRQVIAENLVAQRERLQQDLQTLAKLHDVEMKGLSDTIASLKLQQEQLRLQLSHRRKQVALAKLQLDKLNAMHLEGYASNRQLEEQESNLLDSQARYQEYQRQLLDTSQKIVQAEQQLHEKPLDDEKKRNDIERQLADNRQSMAENEARRSIELRAPKSGYVGMIMVKNGQMLNAGQSAIAILPNNTNLVARIMVNTQSIGFIQPGQRVVLRYKAFPYQKFGQQYGKVIEVSRTALSPQEVTTLTGKNNVQEQQYRVLVSLDKQTISAYSQNEKLKPGMALDADFIVDKRRLYEWVLEPIFALGHKISL

SEQ ID NO: 206 - protein, 418 aa, Pseudomonas sp.
MFRQEALDAQHAGGLGEIVLIRPVSFTFLTLLAAAMALLVVGFFLFGSYTKRSTVSGQLVPASGQVKVHAPQAGIVLRKFVQEGQAVRRGERLMVLSSERYGSDAGPVQAGISRRLEQRRDSLRDELEKLRRLQDDERDSLTSKVASLQRELTTLAAQTDSQQRLLALASDAAARYQGLMDKGYISMDQLQQRQAELLGQRQTLQGLERERTSLRQQLTERRNELAGLSARQANQLAETRRQLSAVEQDLAESEAKRTLLVTAPESGIATAVLAEAGQTVDSSRPLLSIVPADTPLQAELYAPSKSIGFIRPGDAVLIRYQAYPYQKFGQYHGKVQSISRASVSYAELSSMVGGVPGLGQDGEQLYRLRVTLDDQAVTAYGQPRPLQSGMLLDADILQDTRRLYEWVLEPLYSLTGKL

SEQ ID NO: 207 - protein, 418 aa, Pseudomonas sp.
MFRQEALDAQHAGGLGEIVLIRPVSFTFLTLLAAAMALLVVGFFLFGSYTKRSTVSGQLVPANGQVKVHAPQAGIVLRKFVQEGQAVRRGERLMVLSSERYGSDAGPVQAGISRRLEQRRDSLRDELEKLRRLQDDERDSLTSKVASLQRELTTLAAQTDSQQRLLALASDAAARYQGLMDKGYISMDQLQQRQAELLGQRQTLQGLERERTSLRQQLTERRNELAGLSARQANQLAETRRQLSAVEQDLAESEAKRTLLVTAPESGIATAVLAEAGQTVDSSRPLLSIVPADTPLQAELYAPSKSIGFIRPGDAVLIRYQAYPYQKFGQYHGKVQSISRASVSYAELSSMVGGVPGLGQDGEQLYRLRVTLDDQAVTAYGQPRPLQSGMLLDADILQDTRRLYEWVLEPLYSLTGKL

SEQ ID NO: 208 - protein, 418 aa, Pseudomonas sp.
MFRQEALDAQHAGALGEIVLIRPVSFTFLTLLAAAMALLVVGFFLFGSYTKRSTVSGQLVPASGQVKVHAPQAGIVLRKFVQEGQAVRRGERLMVLSSERYGSDAGPVQAGISRRLEQRRDSLRDELEKLRRLQDDERDSLTSKVASLQRELTTLAAQTDSQQRLLALASDAAARYQGLMDKGYISMDQLQQRQAELLGQRQTLQGLERERTSLRQQLTERRNELAGLSARQANQLAETRRQLSAVEQDLAESEAKRTLLVTAPESGIATAVLAEAGQTVDSSRPLLSIVPADTPLQAELYAPSKSIGFIRPGDAVLIRYQAYPYQKFGQYHGKVQSISRASVSYAELSSMVGGVPGLGQDGEQLYRLRVTLDDQAVTAYGQPRPLQSGMLLDADILQDTRRLYEWVLEPLYSLTGKL

SEQ ID NO: 209 - protein, 418 aa, Pseudomonas sp.
MLFRPEALAAQKRSTLGTVLLISPVSMRLAAAVALLFCICMGLFLTFATYTKRTAASGVVLPEKGLIRIYAPQAGVVNNLGISEGQPVNADAVLMALSSDTQNGEAGGAQRAISLSIKRRQESLTQEIDKTLALHQQELEGKKRQVEALETEQQKILAQIDLARQRLALTKGIAERYANLQRQDYVSRDQLQEKEDAVLDMRLRGEELNRSLLTVRAESTRLRAELVELPFNHSKQLADLQRRLAENQDQLVESETKREVLITAPTAGEATAIAVSNGSRVDSARPLLSIVPANAKLHAELYLPSRSVGFVRAGDTVMLRYQAYPYQRFGLQPGRVSSISRTALPAEEVMTLGNVSEQMREQGPFYRVTVALASQVIEGQGKRERLRSGMQLDADIMQEKLPLYEWLLEPLRGIGKRL

SEQ ID NO: 210 - protein, 418 aa, Pseudomonas sp.
MFRQEALDAQHAGGLGEIVLIRPVSFTFLTLLAAAMALLVVGFFLFGSYTKRSTVSGQLVPASGQVKVHAPQAGIVLRKFVQEGQAVRRGERLMVLSSERYGSDAGPVQAGISRRLEQRRDSLRDELEKLRRLQDDERDSLTSKVASLQRELTTLAAQTDSQRRLLALASDAAARYQGLMDKGYISMDQLQQRQAELLGQRQTLQGLERERTSLRQQLTERRNELAGLSARQANQLAETRRQLSAVEQDLAESEAKRTLLVTAPESGIATAVLAEAGQTVDSSRPLLSIVPADTPLQAELYAPSKSIGFIRPGDAVLIRYQAYPYQKFGQYHGKVQSISRASVSYAELSSMVGGVPGLGQDGEQLYRLRVTLDDQAVTAYGQPRPLQSGMLLDADILQDTRRLYEWVLEPLYSLTGKL

SEQ ID NO: 211 - protein, 408 aa, Pseudomonas sp.
MYQALGKVTLTRPVSFTFLTLLAAAMALLVVGFFLFGSYTKRSTVSGQLVPASGQVKVHAPQAGIVLRKFVQEGQAVRRGERLMVLSSERYGSDAGPVQAGISRRLEQRRDSLRDELEKLRRLQDDERDSLTSKVASLQRELTTLAAQTDSQQRLLALASDAAARYQGLMDKGYISMDQLQQRQAELLGQRQTLQGLERERTSLRQQLTERRNELAGLSARQANQLAETRRQLSAVEQDLAESEAKRTLLVTAPESGIATAVLAEAGQTVDSSRPLLSIVPADTPLQAELYAPSKSIGFIRPGDAVLIRYQAYPYQKFGQYHGKVQSISRASVSYAELSSMVGGVPGLGQDGEQLYRLRVTLDDQAVTAYGQPRPLQSGMLLDADILQDTRRLYEWVLEPLYSLTGKL

SEQ ID NO: 212 - protein, 417 aa, Pseudomonas sp.
MLFRSEAVHARRDKELGSVLLVAPVPLRWAALAALLFSVLLIVFLTCASYTRRATVTGVLVPQGGLIKVFAPQAGVVQGLAAVEGQTVEAGATLMALGSDLYAEAGGAQVAISRLIKRRNDSLAEEIATTRALHLQELEGKRRRLAVLESEQHKVVTQMDISRQRLGLAQGIAARYADLQRQDYVSRDQLQEKQDAVLEVRLRSEELSRSLLSLRNDIATLRAELAELPFNQGKQLAELERRLSQSQGSLLESEVKRQVVITAPASGEVTAIGVVNGTRADSNRPLLSIVPKDAQLYAELYVPSRSVGFVRPGDAVLLRYQAYPYQHFGLARGTVASVSRSALPADEIMSVGGVSEQQREQGPLYRVRVTLEQQSMLGRGLNERLRSGMQLDADILQENMPLYEWLLEPLRGIGKRL

SEQ ID NO: 213 - protein, 418 aa, Pseudomonas sp.
MLFRPEALDAQKRSTLGTVLLVSPLSMRLAAAVALLFCISMGLFLTFATYTKRTAASGILLPETGLIRIYSPQAGVITRLDIVEGQHVTDGAVLMALSSDTQSGEAGGAQRAISLSIKRRQESLAQEIDETLALHLQELEGKKRQVAALETEQQKILAQIDLTRQRLALTKGLAERYANLQRQDYVSRDQLQEKQDAVLDMRLRGEELNRSLLTVRAESTRLRAELVELPFNQSKQLADLRRRLAENQDRLIESETKREIFITSPTLGEATAIAVSNGSRVDSTRPLLSIVPADAKLHAELYLPSRSVGFVRTGDTVMLRYQAYPYQRFGLQSGRVSSISRTALPADEVMTLGNVSEPMREQGPFYRVTVALASQTIDGQGMHERLRSGMQLDADIMQERLPLYEWMLEPLRGIGKRL

SEQ ID NO: 214 - protein, 418 aa, Pseudomonas aeruginosa
MFRQEALDAQHAGGLGEIVLIRPVSFTFLTLLAAAMALLVVGFFLFGSYTKRSTVSGQLVPASGQVKVHAPQAGIVLRKFVQEGQAVRRGERLMVLSSERYGSDAGPVQAGISRRLEQRRDSLRDELEKLRRLQDDERDSLTSKVASLQRELTTLAAQTDSQQRLLALASDAAARYQGLMDKGYISMDQLQQRQAELLGQRQTLQGLERERTSLRQQLTERRNELAGLSARQANQLAETRRQLSTVEQDLAESEAKRTLLVTAPESGIATAVLAEAGQTVDSSRPLLSIVPADTPLQAELYAPSKSIGFIRPGDAVLIRYQAYPYQKFGQYHGKVQSISRASVSYAELSSMVGGVPGLGQDGEQLYRLRVTLDDQAVTAYGQPRPLQSGMLLDADILQDTRRLYEWVLEPLYSLTGKL

SEQ ID NO: 215 - protein, 418 aa, Pseudomonas aeruginosa
MFRQEALDAQHAGGLGEIVLIRPVSFTFLTLLAAVMALLVVGFFLFGSYTKRSTVSGQLVPASGQVKVHAPQAGIVLRKFVQEGQAVRRGERLMVLSSERYGSDAGPVQAGISRRLEQRRDSLRDELEKLRRLQDDERDSLTSKVASLQRELTTLAAQTDSQRRLLALASDAAARYQGLMDKGYISMDQLQQRQAELLGQRQTLQGLERERTSLRQQLTERRNELAGLSARQANQLAETRRQLSAVEQDLAESEAKRTLLVTAPESGIATAVLAEAGQTVDSSRPLLSIVPADTPLQAELYAPSKSIGFIRPGDAVLIRYQAYPYQKFGQYHGKVQSISRASVSYAELSSMVGGVPGLGQDGEQLYRLRVTLDDQAVTAYGQPRPLQSGMLLDADILQDTRRLYEWVLEPLYSLTGKL

SEQ ID NO: 216 - protein, 418 aa, Pseudomonas aeruginosa
MFRQEALDAQHAGGLGEIVLIRPVSFTFLTLLAAAMALLVVGFFLFGSYTKRSTVSGQLVPASGQVKVHAPQAGIVLRKFVQEGQAVRRGERLMVLSSERYGSDAGPVQAGISRRLEQRRDSLRDELEKLRRLQDDERDSLTSKVASLQRELTTLAAQTDSQRHLLALASDAAARYQGLMDKGYISMDQLQQRQAELLGQRQTLQGLERERTSLRQQLTERRNELAGLSARQANQLAETRRQLSAVEQDLAESEAKRTLLVTAPESGIATAVLAEAGQTVDSSRPLLSIVPADTPLQAELYAPSKSIGFIRPGDAVLIRYQAYPYQKFGQYHGKVQSISRASVSYAELSSMVGGVPGLGQDGEQLYRLRVTLDDQAVTAYGQPRPLQSGMLLDADILQDTRRLYEWVLEPLYSLTGKL

SEQ ID NO: 217 - protein, 408 aa, Pseudomonas aeruginosa
MYQALGKVTLTRPVSFTFLTLLAAVMALLVVGFFLFGSYTKRSTVSGQLVPASGQVKVHAPQAGIVLRKFVQEGQAVRRGERLMVLSSERYGSDAGPVQAGISRRLEQRRDSLRDELEKLRRLQDDERDSLTSKVASLQRELTTLAAQTDSQRRLLALASDAAARYQGLMDKGYISMDQLQQRQAELLGQRQTLQGLERERTSLRQQLTERRNELAGLSARQANQLAETRRQLSAVEQDLAESEAKRTLLVTAPESGIATAVLAEAGQTVDSSRPLLSIVPADTPLQAELYAPSKSIGFIRPGDAVLIRYQAYPYQKFGQYHGKVQSISRASVSYAELSSMVGGVPGLGQDGEQLYRLRVTLDDQAVTAYGQPRPLQSGMLLDADILQDTRRLYEWVLEPLYSLTGKL

SEQ ID NO: 218 - protein, 418 aa, Pseudomonas aeruginosa
MFRQEALDAQHAGGLGEIVLIRPVSFTFLTLLAAAMALLVVGFFLFGSYTKRSTVSGQLVPASGQVKVHAPQAGIVLRKFVQEGQAVRRGERLMVLSSERYGSDAGPVQAGISRRLEQRRDSLRDELEKLRRLQDDERDSLTSKVASLQRELTTLAAQTDSQQRLLALASDAAARYQGLMDKGYISMDQLQQRQAELLGQRQTLQGLERERTSLRQQLTERRNELAGLSARQANQLAETRRQLSAVEQDLAESEAKRTLLVTAPESGIATAVLAEAGQTVDSSRPLLSIVPADTPLQAELYAPSKSIGFIRPGDAVLIRYQAYPYQKFGQYHGKVQSISRASVSYAELSSMVGGVPGLGQDGEQLYRLRVTLDDQAVTAYGQPRPLQSGMLLDADILQDTRRLYEWLLEPLYSLTGKL

SEQ ID NO: 219 - protein, 418 aa, Pseudomonas aeruginosa
MFRQEALDAQHAGSLGRIVLIRPVSFTFLTLLAAAMALLVVGFFLFGSYTKRSTVSGQLVPASGQVKVHAPQAGIVLRKFVEEGQAVRRGERLMVLSSERYGSDAGPVQAGISRQLEQRRDSLRDELEKLRRLQDDERASLASKVASLQRELATLAAQTDSQQRLLALASDAAARYQGLMDKGYISMDQLQQRQAELLGQRQTLQGLARERTSLEQQLTERRNELAGLSARQANQLAETRRQLSAVQQDLAESEAKRTLLVTAPESGIATAVLAEAGQTVDSSRPLLSIVPADTPLQAELYAPSKSIGFIRPGDAVLIRYQAYPYQKFGQYHGKVRSISRASVSYAELSSMVGGVPGLGQDGEQLYRLRVTLDDQAVTAYGQPRPLQSGMLLEADILQDTRRLYEWVLEPLYSLTGKL

SEQ ID NO: 220 - protein, 418 aa, Pseudomonas amygdali
MLFRPEALDAQKRSTLGTVLLVSPLSMRLAAAVALLFCISMGLFLTFATYTKRTAASGVLLPETGLIRIYSPQAGVITRLDIVEGQHVTDGAVLMALSSDTQSGEAGGAQRAISLSIKRRQESLAQEIDETLALHLQELEGKKRQVAALETEQQKILAQIDLTRQRLALTKGLAERYANLQRQDYVSRDQLQEKQDAVLDMRLRGEELNRSLLTVRAESTRLRAELVELPFNQSKQLADLRRRLAENQDRLIESETKREIFITAPTLGEATAIAVSNGSRVDSTRPLLSIVPADAKLHAELYLPSRSVGFVRTGDTVMLRYQAYPYQRFGLQPGRVSSISRTALPADEVMTLGNVSEPMREQGPFYRVTVALASQAIDGQGMHERLRSGMQLDADIMQERLPLYEWMLEPLRGIGKRL

SEQ ID NO: 221 - protein, 417 aa, Pseudomonas sp.
MFRSEALAARQSTWLGDIVLVRPVSFTVMTVVALLLALMVVSFFFYGSYTRRSTVPGQLVPSSGQLKIHSSQYGVVLERYVEEGQQVEQGGRLFLISSERSVDSGPVQAEVSDQLQGQRRSLEEELRKQQQLQVEARQSLDSKLRSLTQELDTLAQQIASQQRLVQLAGNAAERYQGLMDKGYISMDQLQQRQAELLGQRQSLQGLVRESTVLRQQLVERQHERAGLEALHGNQLASIRRSLSSVQQALIESEAKRSLVITAPQPGVATAILVGPGQVVDSSRALMSLVPADANLQAELYAPSKAIGFIQAGDAVLLRYQAYPYQKFGQHHGQVISVSRTTLSAAELANVVGSVPGLGGNGEQIYRIRVAIDRQSVQAYGESRALQAGMLVEADVLQETRRLYEWVLEPLYSLTGKL

SEQ ID NO: 222 - protein, 418 aa, Pseudomonas syringae
MLFRPEALAAQKRSTLGTVLLVSPVSMRLAAAVALLFCMCMGLFLSFATYTKRTAASGVLLPETGLIRIYAPQTGVVNNLSIAEGQPVNADAVLMALSSDTQNGETGGAQRAISLSIKRRQESLTQEIDKTLALHQQELEGKKRQVEALETEQQKILAQIDLARQRLALTKGIAERYANLQRQDYVSRDQLQEKEDAVLDMRLRGEELNRSLLTVRAESTRLRAELVELPFNQSKQLADLQRRLAENQDQLVESETKREILITAPTAGEATALSVSNGSRVDSARPLLSIVPADAKLHAELCLPSRSVGFVRAGDTVMLRYQAYPYQRFGLQPGTVSSISRTALPADEVMTLGNVSEQMREQGPFYRVTVALASQTIEGQGKRDRLRSGMQLDADIMQEKLPLYEWLLEPLRGIGKRL

SEQ ID NO: 223 - protein, 420 aa, Raoultella sp.
MNHDLFRQESLDANKTKVLGPVALYCPPFRWLIVAMVCALTIVLVGFCSLGSYTKRETAKGVLTPESGIMTITALTAGTVTALPVREGAGVKKGERIATVSSEISTARYGQTREAIARQLEIQSQGLTQQLTNLEQRNAEALKSLQERSSLLAQQTTELDTIYRQRQRQIALSQKQVDKMAAMRAEGYASNTQVEQQESDLLDAKVRLQDVARQRIEIRQQHAQTRQQLREQPLTYFQQKNDLQQKLSDITQSMMENESRRSVDLRAPEEGTVSAVLVKPGQIVSAGQTIAMLLPDNAHLQARILLSSRAIGFIHTGQRVVLRYESFPWQKFGQHSGAVSEISTSPLSPQEIAGITGNTQIQEPLYQVKVTLDSQSVQAYGKQIGLRPGSGLDADFIVDKRRIYEWVLEPLNALGKMTSL

SEQ ID NO: 224 - protein, 419 aa, Serratia inhibens
MSDTLFRQEVLDANKSKLIGSVALYCPPYRWLVISIAGFITLSIVIFFIFGSYTKREAAKGELLPLDGVMNIVPMVAGTVIGIPPREGQLVKKGDPLVTISSEVSTAMGQTREKIAEQLKLQQEALEMDLKSLEGLNREAIRGMKERENLLASQMKQLDLQYQQRVRQANLARRQMEKTQLMRAQGYASNVQVEQQETVMLDADAKLQDIARQRIDIQQQLAQTKQQLREQPLNMRNQHNDIERKLSEINQSLTENESRRSFVLRAPDNGMVGTVLVKLGQMVNAGQTAISLLPEDGRLQARIMVSSRAIGFIRPGQKVVLRYQAFPYQKFGQQYGKVTEVSRIALSPQEVSTLTGMGNVQEQHYRVLVELEKQDINVYGKYEKLRPGSAVEADFLIDRRRLYEWVLEPLYALGRNSSV

SEQ ID NO: 225 - protein, 419 aa, Serratia plymuthica
MNNDLFRKESLEANKTKVLGSVALYCPPFRWLIIAIICTLVLALVALFIFGSYTKRETANGMLVPEGGMMNITAINSGMVIALPVSEGDSLKKNDRIATVSSEISTRYGQTREAIARQLDLQQQGLNNQLLNLEQLNGETLKSLQEKERLLQQQSVELDTIYRQRTQQIALANKQLGKMKAMRTQGYASNTQVEQQENVLLDASVRLQDVARQRIDARQQLAQTRQQLREQPITYYQQKNDIQQKLSHITQSMVENESRRSVDLRAPEKGMVSAVLVKTGQIVSAGQTIALMLPDNAHLQARIMLSSRAIGFIHTGQRVVLRYQSFPWQKFGQQYGTVMEISKSTLSPQEVSAITGDNQVQEALYQVKVAIDHQTVQAYGKQIGLLPGSGLQADFIVDKRRIYEWVLEPLNALGKMTSL

SEQ ID NO: 226 - protein, 384 aa, Stenotrophomonas maltophilia
MGLFLVLGQYTRRETVTGQLVPASGLINITALSPGTVARLHVEDGQSIQADEVLMEISSDQDTVRLGATRASIDEQLAEKEGRYRADLVSQDSVARHQREALVARESLIRRQLDQVGEQVALQAEEVEGSQALLERIEPLGKGGYISAFEIQRQQAVVRAARSRKAELQRQGLELQQQLAAAQESLAKLPLEDATRRNEIVRELADVAQSQAQNDLQRGTLFRARQGGVVAALLVKTGQMVSAGQPMITILPGDAKLQAQLLIPSRAIGFIAPGNRVILRYEAFPYQKFGQQYGRVSIVSRSALTPAEHSALTGRLPPADQGPLYRVDVELDRQYVSAYGIKESIRPGMALQADVLIERRRLIEWLFEPLFGIQRRVFEENSNG

SEQ ID NO: 227 - protein, 379 aa, Xanthomonas sp.
MCLGSFTRHEAVNGALVPDRGLLTLTPLSAGIVSNAFVAEGASVRAGEPIVEISGEQDSTSLGDTQASVISQLEIKRGRLNADLQAQNQLYAAQQRDLGRRVALLQAQMDNTAEQIRLQRQRADSALGLYEQWVSAAEKGILTKVQLLQQQDIAIQNQAQLKELQKHALDLRVEHSQLQSQLEQTPATLEAKRNEIARQIADVAQSLSETEARRSVVLRAPTDGIVTNLLVHAGQPVGAQQPLITLLPKDIKLRAELWVPSKAVGFVTRGDNVLLRYQAFPYQKFGRHAGRVVDVSRSAISSKEVSSLLGQQIDDARYRVVVELESQHILANGRQESLRPGMALDADILLEKRRLIEWMFEPVYDMTSRLSDDSAGQRG

SEQ ID NO: 228 - protein, 447 aa, Xanthomonas sp.
MEPCWYEQARGLAAHGDAAASSRQAARAGVGGRQRMTTLFRREAVDYRQRSRLGPVVLRQHGALRWCVGLLMATVILLLAGFFRLGFARSETLYGAVVPAGGLIAVTTPQSGVVVQVGAVQGQRVAAGQVLFVLSAEHRDDRGRPTQRAAVVLAEQQRLAVEAMAQLRAQGRVQQQAAARALAGLRDRLQQIDVELDLLRHRQQLTQSIEQRYRTALTRGLVSQQFVDEKQADVLDQRAHTLELRRERMALADALAQAQAEVQQLPSNLRQQLAVAGAGLQEDRRAAIEQAAASRWEVRAPRAGRVALRPLQRGQAVAQGQRLADLLPTSMATEVVLYAPSRAAGLIGLGMPVQLRFDALPYQHYGQFAGQVVEIAVAPEPPRVDSTSVSEPLYRVRVRLAGDAALRAGHTAVLRPGMRVQGTLALEWRRFSQWAFEPLSSLHGTLR

SEQ ID NO: 229 - protein, 414 aa, Xanthomonas oryzae
MTLFREEVMQARRNSWLGGISLAQPVRAWVLASAAGIVAASVVVFLCMGTYAHRSTVTGQLVPIKGLATVMAPATGVVSRLDVSEGQPVKAGQMLAVVAVPRATLDSGDTQAALQHQLRKRGEGVKSSEQAQLQQLDAQEQGLRAQLANLRQELMQVDAEIATRRNQIALANEVLQRWRQLQDDKYVSVLQIKQQESTALEYTSQMQALQRQATEMRRSAAQIEQQLRVLPGQRGGVQADYRRDAAAVEQEQVQTQVNGALVVTAPVAGVVATQMAKPGQAIQLGQPLLSVVPGDGRLEAELLVPSRAIGFIAPGDTVLLRYQAFPYQKFGHQQGRLSRISRSALSSSELGALIGNVQQGEPYYRVTVGLGRQSVMAYGKPEMLKPGMLLQADIIGERRKLIEWVFEPLLSLGR

SEQ ID NO: 230 - protein, 412 aa, Xanthomonas oryzae
MTRLFRREVMDAQQRSRLGPVVLRQRGALGWCVGVLMATVILLLVGFFCLGFARRQTLYGAVVPADGMIAITTPQSGVVANVGVVQGQRVAAGQVLFVLAAEHRDDRGRPSQQAAAVLAEQQRLTAEAMVQLRAQGRLQQQAAARALAGLRNRLEQVDAELGVLRHRQQLTQSIEQRYRTALTRGLVSQQFVDEKQADVLDQRAHALELQRERLTLADALAQAQAELQQLPVSLRQQLALAGASLQADRRTAIEQAAASRWEVRAPRAGRVALRPLQRGQAVGQGQRLADLLPTSTATEVVLYAPSRAAGLIGPGIPVQLRFDALPYQHYGQFAGRVVEIAAVPEPPRADAPLASEPLYRMRVRLAGDAALRAGHAAVLRPGMRVQGTLALEWRRFSQWAFEPLSSLHGTLR

SEQ ID NO: 231 - protein, 412 aa, Xanthomonas oryzae
MTRLFRREVMDAQQRSRLGPVVLRQRGALGWCVGVLMATVILLLVGFFCLGFARRQTLYGAVVPADGMIAITTPQSGVVANVGVVQGQRVAAGQVLFVLAAEHRDDRGRPSQQAAAVLAEQQRLTAEAMVQLRAQGRLQQQAAARALAGLRNRLEQVDAELGVLRHRQQLTQFIEQRYRTALTRGLVSQQFVDEKQADVLDQRAHALELQRERLTLADALAQAQAELQQLPVSLRQQLALAGASLQADRRTAIEQAAASRWEVRAPRAGRVALRPLQRGQAVGQGQRLADLLPTSTATEVVLYAPSRAAGLIGPGIPVQLRFDALPYQHYGQFAGRVVEIAAAPEPPRADAALASEPLYRVRVRLAGDAALRAGHAAVLRPGMRVQGTLALEWRRFSQWAFEPLSSLHGTLR

SEQ ID NO: 232 - protein, 414 aa, Xanthomonas oryzae
MTLFRQEVLQARRNSWLGGISLAQPVRAWVLASAAGIVAASVVVFLCMGTYAHRSTVTGQLVPIKGLATVMAPATGVVSRLDVSEGQPVKAGQMLAVVAVPRATLDSGDTQAALQHQLRKRGEGLKSSEQAQLQQLDAQEQGLRAQLANLRQELMQVDAEIATRRNQIALANEVLQRWRQLQDDKYVSVLQIKQQESTALEYTSQMQALQRQATEMRRSAAQIEQQLRVLPGQRGGVQADYRRDAAAVEQEQVQTQVNGALVVTAPVAGVVATQMAKPGQAIQLGQPLLSVVPGDGRLEAELLVPSRAIGFIAPGDTVLLRYQAFPYQKFGHQQGLLSRISRSALSSSELGALIGNAQQGEPYYRVTVALARQSVMAYGKPEMLKPGMLLQADIIGERRKLIEWVFEPLLSLGR

SEQ ID NO: 233 - protein, 412 aa, Xanthomonas oryzae
MTRLFRREVMDAQQRSRLGPVVLRQRGALGWCVGVLMATVILLLVGFFCLGFARRQTLYGTVVPADGMIAITTPQSGVVANVGVVQGQRVAAGQVLFVLVAEHRDDRGRPSQQAAAVLAEQQRLTAEAMDQLRAQGRLQQQAAARALAGLRNRLEQVDAELGVLRHRQQLTQSIEQRYRTALTRGLVSQQFVDEKQADVLDQRAHALELQRERLTLADALAQAQAELQQLPVSLRQQLALAGASLQADRRTAIEQAAASRSEVRAPRAGRVALRPLQRGQAVGQGQRLADLLPTSTATEVVLYAPSRAAGLIGPGIPVQLRFDALPYQHYGQFAGRVVEIAAAPEPPRADAALASEPLYRVRVRLAGDAALRAGHAAVLRPGMRVQGTLALEWRRFSQWAFEPLSSLHGTLR

SEQ ID NO: 234 - protein, 403 aa, Xanthomonas oryzae
MDAQQRSRLGPVVLRQRGALGWCVGVLMATVILLLVGFFCLGFARRQTLYGAVVPADGMIAITTPQSGVVANVGVVQGQRVAAGQVLFVLAAEHRDDRGRPSQQAAAVLAEQQRLTAEAMVQLRAQGRLQQQAAARALAGLRNRLEQVDAELGVLRHRQQLTQSIEQRYRTALTRGLVSQQFVDEKQADVLDQRAHALELQRERLTLADALAQAQAELQQLPVSLRQQLALAGASLQADRRTAIEQAAASRWEVRAPRAGRVALRPLQRGQAVGQGQRLADLLPTSTATEVVLYAPSRAAGLIGPGIPVQLRFDALPYQHYGQFAGRVVEIAAVPEPPRADAPLASEPLYRMRVRLAGDAALRAGHAAVLRPGMRVQGTLALEWRRFSQWAFEPLSSLHGTLR

SEQ ID NO: 235 - protein, 414 aa, Xanthomonas oryzae
MTLFREEVMQARRNSWLGGISLAQPVRAWVLASAAGIVAASVVVFLCMGTYAHRSAVTGQLVPIKGLATVMAPATGVVSRLDVSEGQPVKAGQMLAVVAVPRATLDSGDTQAALQHQLRKRGEGVKSSEQAQLQQLDAQEQGLRAQLANLRQELMQVDAEIATRRNQIALANEVLQRWRQLQDDKYVSVLQIKQQESTALEYTSQMQALQRQATEMRRSAAQIEQQLRVLPGQRGGVQADYRRDAAAVEQEQVQTQVNGALVVTAPVAGVVATQMAKPGQAIQLGQPLLSVVPGDGRLEAELLVPSRAIGFIAPGDTVLLRYQAFPYQKFGHQQGRLSRISRSALSSSELGALIGNVQQGEPYYRVTVGLGRQSVMAYGKPEMLKPGMLLQADIIGERRKLIEWVFEPLLSLGR

SEQ ID NO: 236 - protein, 414 aa, Xanthomonas oryzae
MTLFRQEVLQARRNSWLGGISLAQPVRAWVLASAAGIVAGSVVVFLCMGTYAHRSTVTGQLVPIKGLATVMAPATGVVSRLDVSEGQPVKAGQMLAVVAVPRATLDSGDTQAALQHQLRKRGEGLKSSEQAQLQQLDAQEQGLRAQLANLRQELMQVDAEIATRRNQIALANEVLQRWRQLQDDKYVSVLQIKQQESSALEYTSQMQALQRQATEMRRSAAQIEQQLRVLPGQRGGVQADYRRDAAAVEQEQVQTQVNGALVVTAPVAGVVATQMAKPGQAIQLGQPLLSVVPGDGHLEAELLVPSRAIGFIAPGDTVLLRYQAFPYQKFGHQQGLLSRISRSALSSSELGALIGNAQQGEPYYRVTVALARQSVMAYGKPEMLKPGMLLQADIIGERRKLIEWVFEPLLSLGR

SEQ ID NO: 237 - protein, 414 aa, Xanthomonas oryzae
MTLFRQEVLQARRNSWLGGISLAQPVRAWVLASAAVIVAAFVVVFLCMGTYAHRSTVTGQLVPIKGLATVMAPATGVVSRLKVSEGQHVKAGQMLGVVTVPRATLGSGDTQAALQHQLRQRGEGVKSSEQAQLQQLDAQEQGLRAQLANLCQELVQVDAEVATRRNQIALANEVLQRWRQLQDDKYVSVLQIKQQESSALEYTSQMQALQRQATEMRRSAAQIEQQLRVLPGQRGSVQADYRRDAAAVEQEQVQTQVNGALVVTAPVAGVVATQMAKPGQAIQLGQPLLSVVPGDGRLEAELLVPSRAIGFIAPGDTVLLRYQAFPYQKFGHQQGRLSRISRSALSSSELGALIGNAQQGEPYYRVTVALGRQSVMAYGKPEMLKPGMLLQADIIGERRKLIEWVFEPLLSLGK

SEQ ID NO: 238 - protein, 414 aa, Xanthomonas oryzae
MTLFREEVMQARRNSWLGGISLAQPVRAWVLASAAGIVAASVVVFLCMGTYAHRSTVTGQLVPIKGLATVMAPATGVVSRLDVSEGQPVKAGQMLAVVAVPRATLDSGDTQAALQHQLRKRGEGLKSSEQAQLQQLDAQEQGLRAQLANLRQELMQVDAEIATRRNQIALANEVLQRWRQLQDDKYVSVLQIKQQESSALEYTSQMQVLQRQATEMRRSAAQIEQQLRVLPGQRGGVQADYRRDAAAVEQEQVQTQVNGALVVTAPVAGVVATQMAKPGQAIQLGQPLLSVVPGDGRLEAELLVPSRAIGFIAPGDTVLLRYQAFPYQKFGHQQGLLSRISRSALSSSELGALIGNAQQGEPYYRVTVALARQSVMAYGKPEMLKPGMLLQADIIGERRKLIEWVFEPLLSLGR

SEQ ID NO: 239 - protein, 414 aa, Xanthomonas oryzae
MTLFREEVMQARRNSWLGVISLAQPVRAWVLASAAGIVAASVVVFLCMGTYAHRSTVIGQLVPIKGLATVMAPATGVVSRLDVSEGQPVKAGQMLAVVAVPRATLDSGDTQAALQHQLRKRGEGLKSSEQAQLQQLDAQEQGLRAQLANLRQELMQVDAEIATRRNQIALANEVLQRWRQLQDDKYVSVLQIKQQESSALEYTSQMQALQRQATEMRRSAAQIEQQLRVLPGQRGGVQADYRRDAAAVEQEQVQTQVNGALVVTAPVAGVVATQMAKPGQAIQLGQPLLSVVPGDGHLEAELLVPSRAIGFIAPGDTVLLRYQAFPYQKFGHQQGLLSRISRSALSSSELGALIGNAQQGEPYYRVTVALARQSVMAYGKPEMLKPGMLLQADIIGERRKLIEWVFEPLLSLGR

SEQ ID NO: 240 - protein, 412 aa, Xanthomonas oryzae
MTRLFRREVMDAQQRSRLGPVVLRQRGALGWCVGVLMATVILLLVGFFCLGFARRQTLYGAVVPADGMIAITTPQSGVVANVGVVQGQRVAAGQVLFVLAAEHRDDRGRPSQQAAAVLAEQQRLTAEAMVQLRAQGRLQQQAAARALAGLRNRLEQVDAELGVLRHRQQLTQSIEQRYRTALTRGLVSQQFVDEKQADVLDQRAHALELQRERLTLADALAQAQAELQQLPVSLRQQLALAGASLQADRRTAIEQAAASRWEVRAPRAGRVALRPLQRGQAVGQGQRLADLLPTSTATEVVLYVPSRAAGLIGPGIPVQLRFDALPYQHYGQFAGRVVEIAAVPEPPRADAALASEPLYRMRVRLAGDAALRAGHAAVLRPGMRVQGTLALEWRRFSQWAFEPLSSLHGTLR

SEQ ID NO: 241 - protein, 412 aa, Xanthomonas oryzae
MTRLFRREVMDAQQRSRLGPVVLRQRGALGWCVGVLMATVILLLVGFFCLGFARRQTLYGTVVPADGMIAITTPQSGVVANVGVVQGQRVAAGQVLFVLAAEHRDDRGRPSQQAAAVLAEQQRLTAEAMVQLRAQGRLQQQAAARALAGLRNRLEQVDAELGVLRHRQQLTQSIEQRYRTALTRGLVSQQFVDEKQADVLDQRAHALELQRERLTLADALAQAQAELQQLPVSLRQQLALAGASLQADRRTAIEQAAASRWEVRAPRAGRVALRPLQRGQAVGQGQRLADLLPTSTATEVVLYAPSRAAGLIGPGIPVQLRFDALPYQHYGQFAGRVVEIAAAPEPPRADAALASEPLYRVRVRLAGDAALRAGHAAVLRPGMRVQGTLALEWRRFSQWAFEPLSSLHGTLR

SEQ ID NO: 242 - protein, 414 aa, Xanthomonas vasicola
MSLFRQEVLQARRNSWLGGISLAQPVRAWMLASAAVIVAASVVVFLCMGTYAHRSTVTGQLVPIKGLATVMAPATGVVSRLEVSEGQPVKAGQILGVVTVPRATLGSGDTQAALQHQLRQRGEGLKSSEQAQLQQLDAQEQGLHAQLANLRQELVQVDAEIATRRNQIALANEVLQRWRQLQDDKYVSVLQIKQQESSALEYTSQMQALQRQATEMRRSAAQIEQQLRVLPGQRGGVQADYRRDAAAVEQEQVQTQVNGALVVTAPVAGVVATQMAKPGQAIQLGQPLLSVVPGDGRLEAELLVPSRAIGFIAPGDTVLLRYQAFPYQKFGHQQGRLSRISRSALSSSELGALIGNAQQGEPYYRVTVALGRQSVMAYGKPEMLKPGMLLQADIIGERRKLIEWVFEPLLSLGR

SEQ ID NO: 243 - protein, 449 aa, Xanthomonas vasicola
MEPCWYEQTRGLATHGGAPTALPSRRAARPGVGGCGRMTTLFRREAMDYRQRSRLGPVVLGQHRALRWCVGLLMAAVILLLIGFFRLGFARSKTLHGTVVPADGMIALTTPQSGVVVQVGAVQGQVVAAGQMLFVLEAEHRDDRGRPTRRAAAVLAEQQRLAVEAMAQLRAQGRMQQQAAARALAGLRDRLQQVDAELDLLRHRQQLTQSIEQRYRTALTRGLVSQQFVDEKQADVLDQRTHTLEVERERMALADALAQAQVELQQLPSSLRQQLAIAGAGLQEDRRAAIEQAAASRWEVRAPRAGRMALRPLQRGQAVVQGQRLADLLPTSIATEVVLYAPSGAAGLIGPGMPVQLRFDALPYQHYGQFAGRVVEIAAAPEPARVDSASASEPLYRVRVRLAGDAALRTGRMAVLRPGMRVQGTLALEWRRFSQWAFEPLSNVYGTLR

SEQ ID NO: 244 - protein, 414 aa, Xanthomonas vasicola
MTLFRQEVLQARHNSWLGGISLAQPVRAWMLASAAVIVAASVVVFLCLGTYAHRSTVTGHLVPIKGLSTVMAPATGVVSRLEVSEGQPVKAGQILGVVTVPRATLGSGDTQAALQHQLRQRGEGLKSSEQAQLQQLDAQEQGLRAQLANLRQELVQVDAEIATRRNQIALANEVLQRWRQLQDDKYVSVLQIKQQESSALEYTSQMQALQRQATEMRRSAAQIEQQLRVLPGQRGGVQADYRRDAAAVEQEQVQTQVNGALVVTAPVAGVVATQMAKPGQAIQLGQPLLSVVPGDGRLEAELLVPSRAIGFIAPGDTVLLRYQAFPYQKFGHQQGRLSRISRSALSSSELGALIGNVQQGEPYYRVTVALGRQSVMAYGKPEMLKPGMLLQADIIGERRRLIEWVFEPLFSLDK

SEQ ID NO: 245 - protein, 449 aa, Xanthomonas vasicola
MEPCWYEQTRGLATHGGAPTALPSRRAARPGVGGCGRMTTLFRREAMDYRQRSRLGPVVLRQHRALRWCVGLLMAAVILLLIGFFRLGFARSKTLHGTVVPADGMIALTTPQSGVVVQVGAVQGQVVAAGQMLFVLEAEHRDDRGRPTRRAAAVLAEQQRLAVEAMAQLRAQGRMQQQAAARALAGLRDRLQQVDAELDLLRHRQQLTQSIEQRYRTALTRGLVSQQFVDEKQADVLDQRTHTLEVERERMALADALAQAQAELQQLPSSLRQQLAIAGAGLQEDRRAAIEQAAASRWEVRAPRAGRMALRPLQRGQAVVQGQRLADLLPTSIATEVVLYAPSGAAGLIGPGMPVQLRFDALPYQHYGQFAGRVVEIAAAPEPARVDSASASEPLYRVRVRLAGDAALRAGRMAVLRPGMRVQGTLALEWRRFSQWAFEPLSNVYGTLR

SEQ ID NO: 246 - protein, 449 aa, Xanthomonas vasicola
MEPCWYEQTRGLATHGGAPTALPSRRAARPGVGSCGRMTTLFRREAMDYRQRSRLGPVVLRQHRALRWCVGLLMAAVILLLIGFFRLGFARSKTLHGTVVPADGMIALTTPQSGVVVQVGAVQGQVVAAGQMLFVLEAEHRDDRGRPTRRAAAVLAEQQRLAVEAMAQLRAQGRMQQQAAARALAGLRDRLQQVDAELDLLRHRQQLTQSIEQRYRTALTRGLVSQQFVDEKQADVLDQRTHTLEVERERMALADALAQAQAELQQLPSSLRQQLAIAGAGLQEDRRAAIEQAAASRWEVRAPRAGRMALRPLQRGQAVAQGQRLADLLPTSIATEVVLYAPSGAAGLIGPGMPVQLRFDALPYQHYGQFAGRVVEIAEAPEPARVDSASASEPLYRVRVRLAGDAALRTGRMAVLRPGMRVQGTLALEWRRFSQWAFEPLSNVYGTLR

SEQ ID NO: 247 - protein, 420 aa, Xylella fastidiosa
MKDLFRKEVLEAKRTSWLGGISLIQPIKAWLLVFASSVMALLVVVFLCVGTYTRRSTVTGQLVPSKGLVTVVAPATGMVSHIKVSEGANALSGQILALVTVPRTTLASGDAIRALEQQLQERKQGLSMGQQAQAQVLSAQTSGIEAQLSAAQRELAQIEIEIATRQKQIQIANETLQRLKQLEDARYVGTLQIKQQESTVLDYTSQMQAMQRQAMMTRRSIAQLQQSLRELPGQYQNSQATYRRELAQLEQEKVETEARGALSVVSPIDGIVATQIVKSQQTVQAGQPMLSLLPVNGELEAELLLPSSAIGFIEQGDEVRLRYQAYPYQKFGHQRGQVSRISRSALTSSELAALIGNTQQTEPYYRVVVKLPQQSITVYGRHEPLKPGMLIDADILSEKRRLIEWIFEPLYSLRGKVSDN

SEQ ID NO: 248 - protein, 420 aa, Xylella fastidiosa
MKDLFRKEVLEAKRTSWLGGISLIQPIKAWLLVLASSVMALLVVVFLYVGTYTRRSSVTGQLVPSKGLVTVVAPATGMVSQIKVSEGANALSGQILALVTVPRTTLASGDATRALEQQLQERKQGLSMGQQAQAQVLSAQTSGIEAQLSAAQRELAQIEIEIATRQKQIQIANETLQRLKQLEDARYVGTLQIKQQESTVLDYTSQMQAMQRQAIMTRRSIAQLQQSLRELPGQYQNSQATYRRELAQLEQEKVETEARGALSVVSPIDGIVATQIVKSQQTVQAGQPMLSLLPVNGELEAELLLPSSAIGFIEQGDEVRLRYQAYPYQKFGHQRGQVSRITRSALTSNELGALIGNTQQTQPYYRVIVKLPQQSITVYGRHEPLKPGMLIDADILSEKRRLIEWIFEPLYSLRGKVSDN

SEQ ID NO: 249 - protein, 420 aa, Xylella fastidiosa
MKDLFRKEVLEAKRTSWLGGISLIQPIKAWLLVFASSVMALLVVVFLCVGTYTRRSSVTGQLVPSKGLVTVVAPATGMVSQIKVSEGANALSGQILALVTVPRTTLASGDAIRALEQQLQERKQGLSMGQQAQAQVLSAQTSGIEAQLSAAQRELAQIEIEIATRQKQIQIANETLQRLKQLEDARYVGTLQIKQQESTVLDYTSQMQAMQRQAIMTRRSIAQLQQSLRELPGQYQNSQATYRRELAQLEQEKVETEARGALSVVSPIDGIVATQIVKSQQTVQAGQPMLSLLPVNGELEAELLLPSSAIGFIEQGDEVRLRYQAYPYQKFGHQRGQVSRISRSALTSSELAALIGNTQQTEPYYRVIVKLPQQSITVYGRHEPLKPGMLIDADILSEKRRLIEWIFEPLYSLRGKVSDN

SEQ ID NO: 250 - protein, 424 aa, Escherichia coli
MFRQDALENRKMKWQGRAILLPGIPLWLIMLGSIVFITAFLMFIIVGTYSRRVNVSGEVTTWPRAVNIYSGVQGFVVRQFVHEGQLIKKGDPVYLIDISKSTRSGIVTDNHRRDIENQLVRVDNIISRLEESKKITLDTLEKQRLQYTDAFRRSSDIIQRAEEGIKIMKNNMENYRNYQAKGLINKDQLTNQVALYYQQQNNLLSLSGQNEQNALQITTLESQIQTQAADFDNRIYQMELQRYELQKELVNTDVEGEIIIRALTDGKVDSLSVTVGQMVNTGDSLLQVIPENIENYYLILWVPNDAVPYISAGDKVNIRYEAFPAEKFGQFSATVKTISRTPASTQEMLTYKGAPQNTPGASVPWYKVIAMPEKQIIRYDEKYLPLENGMKAESTLFLEKRRIYQWMLSPFYDMKHSATGPLND

SEQ ID NO: 251 - protein, 425 aa, Enterobacterales sp.
MFRQEALQHRNSVWKAKAILLPGIPLWCIVLSCFVFITFFIVFIISGSYTRRINVSAEVTTWPRPIVISTSQQGYVVRSYVKEGQKIRKGDRLYELDTSKTTRSGVVSDNEFNEITSQLHGIDEIIRNLEKGRDETLQSIKDQRNKYKEAYDISSSIVRTAEQGLQKMENNMRNYEQYRKNGLINNDQLTNQMSLYYQQENSVLSIKNQNIQNALQIKNLEREVQIQTSEYDSRIYQMKLQRNELKKELIKSGLNSSIIIRSPSDGIIDTLNVSQGQIVNAGDTLSQIIPERDRKLYLILWIPDMAVPYIKKGDHVRIRYDAFAYEKFGQFNGIIHSISKSPSSQQEMLSYHLLDNIKLFSGHAYYRVMVTPENDRIYYNKEEIHLENGMRAELTLFLENRKIYQWMISPLYDVVKSAQGNIHEK

SEQ ID NO: 252 - protein, 425 aa, Enterobacterales sp.
MFREEALSHSRSKWAGKALLIAGLPAWVIAGISLSFVSVLIIFVTYGGYTRRISVGGEVTTLPRSVNIFATQQGFIAQRFVNVGDQVKKGQHLYQIDVSRVTDSGTVSASTRQAIENQLAQVDSIIAKLEANKKTTLVNLQAQIDQYKLAHSQSKQMVENSRNGVNFMRETMKNYDEYRSKGLINRDQLNNQVYLYYQQQSTFHSLYNQSIQESLQITNLNSDLATRASDFDNQISQYQFQRNDLLRQLAEADASGSLIVNAPADGRVESLSVTPGQMVNTGDSLAQLIPSNHAIYYLVLWLPNSSVPYVSRGDHINVRYDAFPYEKFGQFPGVIESIAYVPASIQEMSTYSSSPTRQSAELVESYYKVLVAIDNTQFGYQGKKLNLSSGMKAQATLFLEKRPIYQWMFAPFYDMKNSVSGPLRE

SEQ ID NO: 253 - protein, 424 aa, Enterobacteriaceae sp.
MFRQDALENRKMKWQGRAILLPGIPLWLIMLGSIVFITAFLMFIIVGTYSRRVNVSGEVTTWPRAVNIYSGVQGFVVRQFVHEGQLIKKGDPVYLIDISKSTRNGIVTDNHRRDIENQLVRVDNIISRLEESKKITLDTLEKQRLQYTDAFRRSSDIIQRAEEGIKIMKNNMENYRYYQSKGLINKDQLTNQVALYYQQQNNLLSLSGQNEQNALQITTLESQIQTQAADFDNRIYQMELQRLELQKELVNTDVEGEIIIRALSDGKVDSLSVTVGQMVNTGDSLLQVIPENIENYYLILWVPNDAVPYISAGDKVNIRYEAFPSEKFGQFSATVKTISRTPASTQEMLTYKGAPQNTPGASVPWYKVIATPEKQIIRYDEKYLPLENGMKAESTLFLEKRRIYQWMLSPFYDMKHSATGPIND

SEQ ID NO: 254 - protein, 424 aa, Enterobacteriaceae sp.
MFRQDALENRKMKWQGRAILLPGIPLWLIMLGSIVFITAFLMFIIVGTYSRRVNVSGEVTTWPRAVNIYSGVQGFVVRQFVHEGQLIKKGDPVYLIDISKSTRNGIVTDNHRRDIENQLVRVNNIISRLEESKKITLDTLEKQRLQYTDAFRRSSDIIQRAEEGIKIMKNNMENYRYYQSKGLINKDQLTNQVALYYQQQNNLLSLSGQNEQNALQITTLESQIQTQAADFDNRIYQMELQRLELQKELVNTDVEGEIIIRALSDGKVDSLSVTVGQMVNTGDSLLQVIPENIENYYLILWVPNDAVPYISAGDKVNIRYEAFPSEKFGQFSATVKTISRTPASTQEMLTYKGAPQNTPGASVPWYKVIATPEKQIIRYDEKYLPLENGMKAESTLFLEKRRIYQWMLSPFYDMKHSATGPIND

SEQ ID NO: 255 - protein, 428 aa, Enterobacteriaceae sp.
MARKLYRKEAIEYKKLHWKGKALLLAGMPAWLIVILSSCFLIALISTLILCTFTQRIDVRGEVITLPHSVNVFAPQQGFVLNQYVKVGDIVKKGQKLYEIDISRNTTNGNVSLAQTAVINEKIINAESIITKLIRNKDETLNALDTQLNTIKKSLSETTSMLANTQAGLNKMHQNLSSYDKYLKEGLITKDQYNYQHSLYFQQQSAYQSLVSQKMQLESQITQFTSDKVTKAADFDNQISNQQNQINDYKNQLVESDAKGNVIIKATTDGRIESLAVTKGQMVDNGSSLAQIKPTDNVEYYLILWLPNNSIPYVKPGDTINIRYDAFPADKFGQFPGEVISISSMPASRQEMSEYTNVNNGTNQQELALYKTIVKIKQKSFSYNGKTLYLSNGLKAEAVVFLEERPLYMWMFTPFYKISQSVSGPINE

SEQ ID NO: 256 - protein, 424 aa, Escherichia coli
MFRQDALENRKMKWQGRAILLPGIPLWLIMLGSIVFITAFLMFIIVGTYSRRVNVSGEVTTWPRAVNIYSGVQGFVVRQFVHEGQLIKKGDPVYLIDISKSTRSGIVTDNHRRDIENQLVRVDNIISRLEESKKITLDTLEKQRLQYTDAFRRSSDIIQRAEEGIKIMKNNMENYRNYQAKGLINKDQLTNQVALYYQQQNNLLSLSGQNEQNALQITTLESQIQTQAADFDNRIYQMELQRYELQKELVNTDVEGEIIIRALTDGKVDSLSVTVGQMVNTGDSLLQVIPENIENYYLILWVPNDAVPYISAGDKVNIRYEAFPAEKFGQFSATVKTISKTPASTQEMLTYKGAPQNTPGDSVPWYKVIAMPEKQIIRYDEKYLPLENGMKAESTLFLEKRRIYQWMLSPFYDMKHSATGPLND

SEQ ID NO: 257 - protein, 428 aa, Klebsiella sp.
MLKKIYRKEAIEYKKHHWKGKALLLAGLPAWLVALLSFAFLAILIATTVFCSFTQRIDVQGEVITLPHSVNVYAPQQGFVISQYVKVGDIVTKGQPLYEIDISRNTTTGNVSAVQIEVINEKIANAEDIISKLNHNKEETTISLEKQLKTINDSLKETNRMLANAQAGLKKMHDNLSSYDKYLSDGLITKDQYNYQHSLYFQQQSTYQSLVSQKMQLESQVTQLNSDKITKIADFDNQISSQENQINDYKNQLVESNANGNIIIKATTEGRIESLTVTKGQMVDKGSSLAQIKPTGDIEYYLILWLPNNSIPYVKPGDEINIRYAAFPSDKFGQFPGKILSISSVPTSRQEMSEYTNVTNGTNQQELALYKTIVKIENKTFEYNGKTLSLSNGLKAQAVVFLEERPLYMWMFTPVYKITQSITGPIHD

SEQ ID NO: 258 - protein, 428 aa, Klebsiella sp.
MTRRLYRKEAIEYKRVYWKGKALLLAGMPTWLIIVLSLLFLGLLISAMIFCSFTQRIDVNGEVITLPHSVNVYAPQQGFIVSQYVKVGDLVKKGQPLYEIDISRDTTTGNVSSVQIEVNKEKIANAEEIINKLSQNKQETLSSIEKQLKANKESLKEINRLLANALAGLKKMHDNLSSYDSYLRDGLITKDQYNYQHSLYFQQQSTYQSLVSQKMQLESQITQYNSDKITKIADFDNQITAQQNQINDFKNQLVESNAKGNAIIKATTEGRIESLSVTKGQMVDNGSSLAQIKPTGNIEYYLILWLPNNSIPYVKPGDVINIRYDAFPSDKFGQFPGEVISISSVPAARQEMSEYTNVSNGTNQQELALYKTIVKIKNKTFSYNGKTLKLSNGLKARAVVFLEKRPLYMWMFTPFYKMSQSVSGPIHD

SEQ ID NO: 259 - protein, 428 aa, Klebsiella sp.
MARKLYRKEAIEYKKLHWKGKALLLAGMPAWLIVILSSCFLIALISTLILCTFTQRIDVRGEVITLPHSVNVFAPQQGFVLNQYVKVGDIVKKGQKLYEIDISRNTTNGNVSLAQTAVINEKIINAESIITKLIRNKDETLNALDTQLNTIKKSLSETTSMLANTQAGLNKMHQNLSSYDKYLKEGLITKDQYNYQHSLYFQQQSAYQSLVSQKMQLESQITQFTSDKVTKAADFDNQISNQQNQINDYKNQLVESDAKGNVIIKATTDGRIESLAVTKGQMVDNGSSLAQIKPTGNVEYYLILWLPNNSIPYVKPGDTINIRYDAFPADKFGQFPGEVISISSMPASRQEMSEYTNVNNGTNQQELALYKTIVKIKQKSFSYNGKTLYLSNGLKAEAVVFLEERPLYMWMFTPFYKISQSVSGPINE

SEQ ID NO: 260 - protein, 428 aa, Klebsiella sp.
MARKLYRKEAIEYKKLHWKGKALLLAGMPAWLIVTLSSCFLIALISTLILCTFTQRIDVRGEVITLPHSVNVFAPQQGFVLNQYVKVGDIVKKGQKLYEIDISRNTTNGNVSLAQTAVINEKIINAESIITKLIRNKDETLNALDTQLNTIKKSLSETTSMLANTQAGLNKMHQNLSSYDKYLKEGLITKDQYNYQHSLYFQQQSAYQSLVSQKMQLESQITQFTSDKVTKAADFDNQISNQQNQINDFKNQLVESDAKGNVIIKATTDGRIESLAVTKGQMVDNGSSLAQIKPTGNVEYYLILWLPNNSIPYVKPGDTINIRYDAFPADKFGQFPGEVISISSMPASRQEMSEYTNVNNGTNQQELALYKTIVKIKQKSFSYNGKTLYLSNGLKAEAVVFLEERPLYMWMFTPFYKISQSVSGPINE

SEQ ID NO: 261 - protein, 425 aa, Klebsiella sp.
MFRQEALQHRNSVWKAKAILLPGIPLWCIVLSCFVFITFFIVFIISGSYTRRINVYAEVTTWPRPIVISTSQQGYVVRSYVKEGQKIRKWDRLYELDTSKTTRSGVVSDNEFNEITSQLHGIDEIIRNLEKGRDETLQSIKDQRDKYKEAYDISSSIVRTAEQGLQKMENNMRNYEQYRKSGLINNDQLTNQMSLYYQQENSVLSIKNQNIQNALQIKNLEREVQIQTSEYDSRIYQMKLQRNELKKELIKSGLNSSIIIRSPSDGIIDTLNVSQGQIVNAGDTLSQIIPEEDRKLYLILWIPDMAIPYIKKGDRVKIRYDAFAYEKFGQFNGIIQSISKSPSSQQEMLSYHLLDNIKLFSGHAYYRVMVTPENDRIFYNKEEIHLENGMRAEITLFLENRKIYQWMISPLYDVVKSAQGNIHEK

SEQ ID NO: 262 - protein, 428 aa, Klebsiella sp.
MLRKIYRKEAIEYKKHHWKGKALLLAGIPAWLVALLSFAFLTILIATTILCSFTQRIDVRGEVITLPHSVNVYAPQQGFVISQYAKVGDIVVKGQPLYEIDISRNTTSGNVSASQIEVINEKIANAEDIITKLSHNKDETRLSLDKQLQTINDSLKETNRMLVNAQTGLKKMHDNLSSYDKYLSDGLITKDQYNYQHSLYFQQQSTYQSLVSQKMQLESQITQLNSDRITKIADFDNQISSQENQINDYKNQLVESNANGNVIIKATTEGRIESLAVTKGQMVDKGSSLAQIQPTGDIEYYLILWLPNNSIPYVKPGDIINIRYDAFPADKFGQFPGKILSISSVPTSRQELSEYTNVNNGTNQQELALYKTIVKIENKTFEYNGKTLSLSNGLKAQAVVFLEERPLYMWMFTPVYKITQSITGPIND

SEQ ID NO: 263 - protein, 428 aa, Klebsiella sp.
MLRKIYRKEAIEYKKHHWKGKALLLAGLPAWLIALLSFAFLAILITTTIFCTFTQRIDVRGEVITLPHSVNVYAPQQGFVISQYAKVGDLVAKGQSLYEIDISRNTTTGNVSTAQIEVINEKIANAEDIITKLSHNKEETRLSLDKQLKTINDSLKETNRMLVNAQTGLKKMHDNLSSYDKYLRDGLITKDQYNYQHSLYFQQQSTYQSLVSQKMQLESQVTQLNSDKITKTADFDNQISSQENQINDYKNQLVESNANGNVIIKATTEGRIESLAVTKGQMVDKGSSLAQIKPTGDIEYYLILWLPNNSIPYVKPGDVINIRYDAFPSDKFGQFPGKILSISSVPTSRQELSEYTNVNNGTNQQELALYKTIVKIENKTFNYNGKTLSLSNGLKAQAVVFLEERPLYMWMFTPVYKITQSITGPIHD

SEQ ID NO: 264 - protein, 428 aa, Klebsiella sp.
MARKLYRKEAIEYKKLHWKGKALLLAGMPAWLIVILSSCFLIALISTLILCTFTQRIDVRGEVITLPHSVNVFAPQQGFVLNQYVKVGDIVKKGQKLYEIDISRNTTNGNVSLAQTAVINEKIINAESIITKLIRNKDETLNALNTQLNTIKKSLSETTSMLANTQAGLNKMHQNLSSYDKYLKEGLITKDQYNYQHSLYFQQQSAYQSLVSQKMQLESQITQFTSDKVTKAADFDNQISNQQNQINDYKNQLVESDAKGNVIIKATTDGRIESLAVTKGQMVDNGSSLAQIKPTGNVEYYLILWLPNNSIPYVKPGDTINIRYDAFPADKFGQFPGEVISISSMPASRQEMSEYTNVNNGTNQQELALYKTIVKIKQKSFSYNGKTLYLSNGLKAEAVVFLEERPLYMWMFTPFYKISQSVSGPINE

SEQ ID NO: 265 - protein, 428 aa, Klebsiella sp.
MLKKLYRKEAVEYKKSHWRGKALLLNGLPAWLILLLSTLFLMALISALIFGKFTQRIDVKGEVITLPHSINVFSPQQGFIIKQYVKIGDIVKKDQALYEIDVSRNTSSGNVSAAQIASINDKIENSNDIIKKLIDNKSQTINALNEQLKTSTDSLKETNRMLQNTQAGLKKMHANLASYDKYLSDGLITKDQYNYQHSLYFQQQSAYQSLVSQKMQLESQITQLNSDRVTKAADFDNQISSQYNQANDYKNQLVESNANGNLIIKATSDGRVESLSATQGQTVENGSSLAQIKPTGNVEYYLILWLPNNAIPYLKVGDPINIRYDAFPSDKFGQFPGTILSISSMPASRQEMSEYTNVSDGTAQQELALYKAIVKIKDKEFKHNGKTLRLSNGLKAQAVVFLEERPLYMWMFTPVYKISQSVSGPVND

SEQ ID NO: 266 - protein, 428 aa, Klebsiella sp.
MAQNIYRKEAIEYKKYHWKGKALLLAGMPAWLITLLACTFLIVLIFMLIFFNYTQRINVYGEVVTLPHSINVFAPQQGFVVKQFVNIGDVVKKGTPLYELDVSRHTTSGNVSLAMTEVIDEKISNAKEIIQKITENKKETVDALNDQLTSVESSLHETVRMLANTQAGLKKMRTNLSSYDDYLKKGLITKDQYNYQHNLYFQQQSSYQSLTSQKMQLETQLTQLKSDMVTKSADFDNQISSQKNQINDYKNQRVESSANDNAIIKATADGKIESLAVTSGQMVDRGSSLAQIKPIGNVEYYLMLWLPNNSIPYIKVGDTINIRYEAFPSDKFGQFPGKVATISTVPASRQELQEYNNVGNAANQQDITLYKALVSIEDSSFRYNDKVLTLSNGLKAEATVFLEERPLYMWMFTPFYKISHSVNGPIND

SEQ ID NO: 267 - protein, 428 aa, Klebsiella sp.
MLKKIYRKEAIEYKKHHWKGKALLLAGLPAWLIVLLSFSFLAIFIATTIFCSFTQRIDVKGEVITLPHSVNVYAPQQGFVISQYVKVGDIVSKGQPLYEIDISRNTTTGNVSAAQIEVINEKIANAEDIIRKLSHNKEETSLSLDKQLKTINDSLKETNQMLANAQTGLKKMHDNLASYDKYLSDGLITKDQYNYQHSLYFQQQSTYQSLVSQKMQLESQVTQLNSDKITKTADFDNQISSQENQINDYKNQLVESSANGNIIIKATTEGRIESLAVTKGQMVDKGSSLAQIKPTGDIEYYLILWLPNNSIPYVKPGDIINIRYDAFPADKFGQFPGKILSISSVPTSRQEMSEYTNVTDGTNQQELALYKTIVKIENKSFEYNGKTLSLSNGLKAQAVVFLEERPLYMWMFTPVYKITQSITGPIND

SEQ ID NO: 268 - protein, 429 aa, Klebsiella aerogenes
MNKNLYRKEAIEYKRHHWKGKALLLAGLPAWLIALLSALFLIALICALIFCKFTQRVDVKGEVITLPHSINVFAPQQGFIVKQFVKIGDVVKKNQPLYEIDVSRSTTSGNVSAAQIEVINEKIANSEDIIKKLEDNKNQTLNALNAQLKTTTASLKETNRMLVNTQVGLKKMHDNLSSYDKYLSNGLITKDQYNYQHSLYFQQQSAYQSLVSQKMQLESQITQLNSDIITKAADFDNQISSQYNQANDYKNQLVESNANGNIIIKSTSDGRIESLSATQGQTVDNGSSLAQIKPTGNVEYYLILWLPNNTIPYLKTGDTINIRYDAFPSDKFGQFPGKIISISSMPASRQEMSEYTNVSNGTNQQQELALYKAIVKIKDKQFEYDGKKLSLSNGLKAQAVVFLEERPLYMWMFTPVYKITQSVSGPIHD

SEQ ID NO: 269 - protein, 431 aa, Klebsiella huaxiensis
MAKGIYRKEAIEYKKYHWKGKALLLAGMPAWLITSVSFIFLSILILTLFFCSYTQRIDVRGEVITLPHSINVFSPQQGFIVQQYVKTGDIVKKGTPLYELDISRHTANGNVSDAISEVITEKIINAEEIIKKIAQNKKETLDALNEQVRQFNNSLKETTKMLANTQSGLKKMKDNLASYDLYLRQGLITKDQYNYQHSLYFQQQSTYQSLVSQKMQQESQLTQLKSDIVTKAADFDNQISSQKNNINDFKNQMVETNANGNVIIKATNDGKIESMAVTNGQMVDKGSSLVQIKPTGNVEYYLVLWLPNNSIPYVKKGDIINIRYDAFPSDKFGQFPGIVETISSVPASRQELSEYTNVNISNEMNQQELALYKTLVSIKHKSFNYNGKNLSLSNGLKAQAIVFLEKRPLYMWMFTPFYKISQSVSGPLNDQ

SEQ ID NO: 270 - protein, 428 aa, Klebsiella pneumoniae
MLKKIYRKEAIEYKKHHWKGKALLLAGLPAWLVALLSFAFLAILIATTVFCSFTQRIDVQGEVITLPHSVNVYAPQQGFVISQYVKVGDIVTKGQPLYEIDISRNTTTGNVSAVQIEVINEKIANAEDIISKLNHNKEETTISLEKQLKTINDSLKETNRMLANAQAGLKKMHDNLSSYDKYLSDGLITKDQYNYQHSLYFQQQSTYQSLVSQKMQLESQVTQLNSDKITKIADFDNQISSQENQINDYKNQLVESNANGNIIIKATTEGRIESLTVTKGQMVDKGSSLAQIKPTGDIEYYLILWLPNNSIPYVKPGDVINIRYAAFPSDKFGQFPGKILSISSVPTSRQEMSEYTNVTNGTNQQELALYKTIVKIENKTFEYNGKTLSLSNGLKAQAVVFLEERPLYMWMFTPVYKITQSITGPIHD

SEQ ID NO: 271 - protein, 428 aa, Klebsiella pneumoniae
MARKLYRKEAIEYKKLHWKGKALLLAGMPAWLIVTLSSCFLIALISTLILCTFTQRIDVRGEVITLPHSVNVFAPQQGFVLNQYVKVGDIVKKGQKLYEIDISRNTTNGNVSLAQTAVINEKIINAESIITKLIRNKDETLNALDTQLNTIKKSLSETTSMLANTQAGLNKMHQNLSSYDKYLKEGLITKDQYNYQHSLYFQQQSAYQSLVSQKMQLESQITQFTSDKVTKAADFDNQISNQQNQINDYKNQLVESDAKGNVIIKATTDGRIESLAVTKGQMVDNGSSLAQIKPTGNVEYYLILWLPNNSIPYVKPGDTINIRYDAFPADKFGQFPGEVISISSMPASRQEMSEYTNVNNGTNQQELALYKTIVKIKQKSFSYNGKTLYLSNGLKAEAVVFLEERPLYMWMFTPFYKISQSVSGPINE

SEQ ID NO: 272 - protein, 428 aa, Klebsiella pneumoniae
MARKLYRKEAIEYKKLHWKGKALLLAGMPAWLIVILSSCFLIALISTLILCTFTQRIDVRGEVITLPHSVNVFAPQQGFVLNQYVKVGDIVKKGQKLYEIDISRNTTNGNVSLAQTAVINEKIINAESIITKLIRNKDETLNALNTQLNTIKKSLSETTSMLANTQAGLNKMHQNLSSYDKYLKEGLITKDQYNYQHSLYFQQQSVYQSLVSQKMQLESQITQFTSDKVTKAADFDNQISNQQNQINDYKNQLVESDAKGNVIIKATTDGRIESLAVTKGQMVDNGSSLAQIKPTGNVEYYLILWLPNNSIPYVKPGDTINIRYDAFPADKFGQFPGEVISISSMPASRQEMSEYTNVNNGTNQQELALYKTIVKIKQKSFSYNGKTLYLSNGLKAEAVVFLEERPLYMWMFTPFYKISQSVSGPINE

SEQ ID NO: 273 - protein, 428 aa, Klebsiella pneumoniae
MLKKIYRKEAIEYKKHHWKGKALLLAGLPAWLVALLSFAFLAILIATTVFCSFTQRIDVQGEVITLPHSVNVYAPQQGFVISQYVKVGDIVTKGQPLYEIDISRNTTTGNVSAVQIEVINEKIANAEDIISKLNHNKEETTISLEKQLKTINDSLKETNRMLANAQAGLKKMHDNLSSYDKYLSDGLITKDQYNYQHSLYFQQQSTYQSLVSQKMQLESQVTQLNSDKITKIADFDNQISPQENQINDYKNQLVESNANGNIIIKATTEGRIESLTVTKGQMVDKGSSLAQIKPTGDIEYYLILWLPNNSIPYVKPGDEINIRYAAFPSDKFGQFPGKILSISSVPTSRQEMSEYTNVTNGTNQQELALYKTIVKIENKTFEYNGKTLSLSNGLKAQAVVFLEERPLYMWMFTPVYKITQSITGPIHD

SEQ ID NO: 274 - protein, 428 aa, Klebsiella pneumoniae
MLRKIYRKEAIEYKKHHWKGKALLLAGIPAWLVALLSFAFLTILIATTILCSFTQRIDVRGEVITLPHSVNVYAPQQGFVISQYAKVGDIVVKGQPLYEIDISRNTTSGNVSASQIEVINEKIANAEDIITKLSHNKDETRLSLDKQLQTINDSLKETNRMLVNAQTGLKKMHDNLSSYDKYLSDGLITKDQYNYQHSLYFQQQSTYQSLVSQKMQLESQITQLNSDRITKIADFDNQISSQENQINDYKNQLVESNANGNVIIKATTEGRIESLAVTKGQMVDKGSSLAQIQPTGDIEYYLILWLPNNSIPYVKPGDDINIRYDAFPADKFGQFPGKILSISSVPTSRQELSEYTNVNNGTNQQELALYKTIVKIENKTFEYNGKTLSLSNGLKAQAVVFLEERPLYMWMFTPVYKITQSITGPIND

SEQ ID NO: 275 - protein, 428 aa, Klebsiella pneumoniae
MTNSIYRKEAIAYKKLHWKGKALLLAGLPAWLVTCLSLLFLCALICALIFCKFTQRIDVKGEVITLPHSVNVFSPQQGFVVNQYVQIGDVVKKGQTLYELDVSRNTTTGNVSAAQIEVINEKIANSEAIIKKLTHNKNETLIALDAQLKNARNSLNETVRMLANTQQGLSKMHENLSSYDKYLKEGLITKDQYNYQHSLYFQQQSAYQSLISQKMQLETQLTQLSSDKVTKAADFDNQISSQYNQTNDYKNQLVESNANGNIIIKATTEGRIESLAVTKGQMVDKGSSLAQIKPIGNIEYYLILWLPNNSIPYVKVGDTINIRYDAFPSDKFGQFPGEIISISSLPASRQEMSEYTNVNDGTNQQELALYKAIVKIRDKKFNYDGKELSLSNGLKAQAVVFLEERPLYMWMFTPVYKISQSVSGPVND

SEQ ID NO: 276 - protein, 428 aa, Klebsiella sp.
MARKLYRKEAIEYKKLHWKGKALLLAGMPVWLIVILSSCFLIALISTLILCTFTQRIDVRGEVITLPHSVNVFAPQQGFVLNQYVKVGDIVKKGLKLYEIDISRNTTNGNVSLAQTAVINEKITNAESIITKLIRNKDETLNALDTQLNTIKKSLSETTSMLANTQAGLNKMHQNLSSYDKYLKEGLITKDQYNYQHSLYFQQQSAYQSLVSQKMQLESQITQFTSDKVTKAADFDNQISNQQNQINDYKNQLVESDAKGNVIIKATTDGRIESLAVTKGQMVDNGSSLAQIKPTGNVEYYLILWLPNNSIPYVKPGDTINIRYDAFPADKFGQFPGEVISISSMPASRQEMSEYTNVNNGTNQQELALYKTIVKIKQKSFSYNGKTLYLSNGLKAEAVVFLEERPLYMWMFTPFYKISQSVSGPINE

SEQ ID NO: 277 - protein, 424 aa, Klebsiella variicola
MYRKEAIEYKKHHWKGKALLLAGLPAWLIVLLSFSFLAILIATTIFCSFTQRIDVKGEVITLPHSVNVYAPQQGFVISQYAKVGDIVNKGQPLYEIDISRNTTTGNVSAAQIEVINEKIANAEDIISKLSHNKEETSLSLDKQLKTINDSLKETNQMLANAQTGLKKMHDNLASYDKYLSDGLITKDQYNYQHSLYFQQQSTYQSLVSQKMQLESQVTQLNSDKITKTADFDNQISSQENQINDYKNQLVESSANGNIIIKATTEGRIESLAVTKGQMVDKGSSLAQIKPTGDIEYYLILWLPNNSIPYVKPGDVINIRYDAFPADKFGQFPGKILSISSVPTSRQEMSEYTNVTDGTNQQELALYKTIVKIENKTFEYNGKTLSLSNGLKAQAVVFLEERPLYMWMFTPVYKITQSITGPIND

SEQ ID NO: 278 - protein, 424 aa, Kosakonia oryzae
MLFRKEVAENIRSHQSGQVLLLTGWPVWLTIAVTLLFIAALLLFLIYGNYTRQVNVSGEINSWPHTTDLFAPEEGVIARVFITPGQTVRAGAALYELDVSKTSPSGNLSSTTLAVLENQSVQIDKALRNIDYNQHVTLESLQKQLDQLSIERQKTVSMVDISAEGVNAMRSSMKDYERYRSQGLVTTDQQNNQRYLFYQQMSVWNSLSSQIVQQDMQINSLRSDLVTRAAQFDEQKSRYLLQQADIKRQLAEANVSKTRLITAPAAGHVSSLNITQGQMVAAGDEVVQLVPSTGILFHLVLWLPDDSVPYIKPGEDINIRYAAYPAEKYGQFPGKIISVSSAPVTGSELRKRASIPQSPGKIWFKAVVAVDKEKLQWRGKRLPLVSGMQAQATLFLEKRPLYQWMFYPYYTLKNSISEPVSDKA

SEQ ID NO: 279 - protein, 424 aa, Kosakonia oryzae
MFRKEALENRKMRWRGRAILLPGVPLWLIIMFCVCFLAAFLTFVIDGTYMRRVNVSGEITTWPRPVNIYSNVQGFIVKSYVTEGQKIKKGDPVYLIDVSRSTHSGVVSDNQRNDIEQQITRVENIIAGLQESKNATLKTLEKQKQQYSEALKFSTDVIRRAEEGVKLMKENMENYRRYQARGLITKDQLTNHVTLYYQQQNDLLGLRSQSEQNALQVTSLESEIQTQAADFDNRIYQMELQRYDLQKERLNADISGDVIVRATSDGKIDSLSVTAGQMVNTGDSLLQIIPESIDHYLLVAWVPNDAAPYIAVGDKVNLRYEAFPAEKFGQFSATISQISKTPASTQEMMTYPGAPKNVQGTSIPYYKVVIKPDREVVEYDGKRISLENGMKAQSTLFLEKRRIYQWMLSPFYDMKHSATGPVNE

SEQ ID NO: 280 - protein, 424 aa, Kosakonia radicincitans
MFRKEALENRKMRWRGRAILLPGVPLWLIIMFCVCFLAAFLTFVIDGTYMRRVNVSGEITTWPRPVTIYSNAQGFIVKSYVTEGQKIKKGDPVYLIDVSRSTHSGVVSDNQRNDIEQQITRVENIIAGLQESKNATLKTLEKQKQQYSEALKFSTAVIRRAEEGVKLMKENMENYRRYQARGLITKDQLTNHVTLYYQQQNDLLGLRSQSEQNALQVTSLESEIQTQAADFDNRIYQMELQRYDLQKERLNADISGDVIVRATSDGKIDSLSVTAGQMVNAGDSLLQIIPESIDHHLLVAWVPNDATPYIAVGDKVNLRYEAFPAEKFGQFSATISQISKTPASTQEMMTYPGAPKNLQGTSIPYYKVVIKPDREVVEYDGKRISLENGMKAQSTLFLEKRRIYQWMLSPFYDMKHSATGPVNE

SEQ ID NO: 281 - protein, 430 aa, Limnobaculum parvum
MDKRRLFRQEAIEHQKAKWAGTALLITGYPAWIIACASAFFVIVLIISLTFGNYTRRINVNGEIVTQPRAINIFAPQQGFISKLLVDAGVEVTKGTALYQLDVSRIARSGNVTIHTTEAIEKQLVHVEGIIAKLEKNKQATQENIKQQIAQSEDANKKSQSLVNNAAKGVEEMRQSMKNYEEYQRQGLVNKEQFNNQRYLYYQQQSSYQALNTQIIQENLKISNLQSELTTSAADFDNQIAQYQYQLNELQRQLTEADAAGMLIINAPSDGKIESLSVTEGQMVNPGDSLAQLIPTSINAYYLVLWLPNNSLPYVSPGDRINIRYDAFPYQKFGQFPGTVQTVSAVPASIQEMSTYNSAPRAAGGGTDQSYYKVIVALDSHHFMLANKELRLTSGMKAQTTLFLEKRPLYQWMLSPFYDIKKSITGPVNG

SEQ ID NO: 282 - protein, 428 aa, Morganella morganii
MFRKAALDNRKMKWRGKALLLPGIPLWLIIVCSFLFLMVFLTFILSGSYTRRVSVTGEITTNPRAVTVYAGVQGFITGQYVNEGQIIRKGDPVYRIDTSRATASGVVSENQRREIENQLQHIRDMTARLRDNKQTTLALLEKQKSLYAQAQLRSAGIIERAQEGIRIMKQNMDNYHHYRESGLINKDQWLNQAALYYSQQNSLLGLSGQNEQNILQVTSLESQILTQAAEFDNRIYQLELQQSELMKEQVNNDAGGEIIVRALTDGRVDSLGVTVGQMVNPGDTLLQILPENIRQYWLALWVPNDALPYLNVGDAVNIRYEAFPAEKFGQFAATIQVISKTPASAQEMMTYAGAPKVSQMPQSIPYYKVIVRPEKQMIAYDGRELSLQNGMKANTTLFLEKRKIYQWMVSPFYDMKQSASGPTGADTP

SEQ ID NO: 283 - protein, 428 aa, Morganella morganii
MFRKAALDNRKMKWRGKALLLPGIPLWLIIVCSFLFLMVFLTLILSGSYTRRVSVTGEITTNPRAVTVYAGVQGFITGQYVNEGQIIRKGDPVYRIDTSRATASGVVSENQRREIENQLQHIRDMTARLRDNKQTTLASLEKQKSLYAQAQLRSAGIIERAQEGIRIMKQNMDNYHHYRERGLINKDQWLNQAALYYSQQNSLLGLSGQNEQNILQVTSLESQILTQAAEFDNRIYQLELQQSELMKEQVNNDAGGEIIVCALTDGRVDSLGVTVGQMVNPGDTLLQILPENIRQYWLALWVPNDALPYLNVGDAVNIRYEAFPAEKFGQFAATIQVISKTPASAQEMMTYAGAPKVSQMPQSIPYYKVIVRPEKQMIAYDGRELSLQNGMKANTTLFLEKRKIYQWMVSPFYDMKQSASGPTGADTP

SEQ ID NO: 284 - protein, 364 aa, Photobacterium damselae
MFRKEAKLSTNWTGEAILLKGINPIIILFFSIIFISALILFIIVFSYTHRMMVVGEISSSPRPTWIYSNFTGIISERFVQEGDSIKKGASIYKIDVSKKNNIGIVSDNRKRTILIQINKINEINENLKENYDRNTRIIQNKLKQYQESIKFLSLEIKNNKITINEFKNNMINYKSYQKKGLINKDQLISQILNYNQQQSTLTNLIQQENQYKLQVTDLNGELVSNKLKLDNELNNNELKIFDLNKEPINNSSEQSIIIRSPINGVVDSLSINHGQMISVRDSLVQIIPNDISDYYLVLWVSNKIIPYIKKGDPVYIKYDAFPAEKFGQFKGTIFDISHIPATINEMNSYQSSPKLGSTNDRAIF

SEQ ID NO: 285 - protein, 428 aa, Serratia sp.
MRDLFRQEATDHQRAKWAGKALLINGLPAWCFGLLSFLFILVFLSFLIFSHYTRRINVYGEITTFPRSVNVFAPQQGFISERFVEVGDAVKKGQRLYQIDVSRVTDNGKVSANTRLALENQLKHVDSIILKLQDNKRMTLENLRAQKKQYELAHMQSKQLLDNAREGVAFAQDNMRSYKEYQQRGLITKDQLSGQTYSFYQQQSLFQNLHSQHIQESLQITNLESDIVTRAADFDNQISQYEFQRNDLQRQLAEADASGALIVNAPSDGRIESLSVTPGQMVNSGDSLVQIIPRNGAIYQLVLWLPNTSVPYVSAGDGINIRYDAFPYEKFGQFPGRIESIAYVPASIQEMSTYSSSPVHQPSVQTASFYKVTVSLDETHISYQGKALQLTNGMRAQSTLFLEKRPLYQWMFSPFYDMKNSLMGPIND

SEQ ID NO: 286 - protein, 428 aa, Serratia sp.
MRDLFRQEATDHQRAKWAGKALLINGLPAWCFGLLSFLFILVFLSFLIFSHYTRRINVYGEITTFPRSVNVFAPQQGFISERFVEVGDVVKKGQRLYQIDVSRVTDNGKVSANTRLALENQLKHVDSIILKLQDNKRMTLENLRAQKKQYELAHMQSKQLLDNAREGVAFAQDNMRSYKEYQQRGLITKDQLSGQTYSFYQQQSLFQNLHSQHIQESLQITNLESDIVTRAADFDNQISQYQFQRNDLQRQLAEADASGALIVNAPSDGRIESLSVTPGQMVNSGDSLVQIIPRNGAIYQLVLWLPNASVPYVSAGDAINIRYDAFPYEKFGQFPGRIESIAYVPASMQEMSTYSSSPVHQPNAQTASYYKVTVSLDETHISYQGKALQLTNGMRAQSTLFLEKRPLYQWMFSPFYDMKNSLMGPIND

SEQ ID NO: 287 - protein, 424 aa, Serratia marcescens
MFRQEAIDNQKMKWRGRAILLPGIPFWLTAGLCLFFLIAFLTFAIAGTYTRRVNVTGEISTYPRAANVYSAVQGVVVKQFVTEGQVIAAGAPIYQIDVSKSTRSGVVSDNQQRDIDGQLARIAQIISRLESSKQATLTMLEQQKAQYTAAFTRSTDILKRAQEGIRIMKENMENYRHYQTKGLITKDQLTNQVALYYQQQNNLLGLSGQNEQNALQITALESQIHIQAANFDNQIYQMELQRHELQKERLNIDAGGAIIVRALADGRIDSLSVTVGQMVNVGDSLLQVIPHNIAHYALVLWVPNDAIPYIATGDKVNVRYDAFPAEKFGQFAATVSVISKAPASPQEMLTYQGAPKAALTAAVPYYKTIVIPEKQVIAYDGKRLSLENGMKAQSTLFLEKRQIYQWMLSPFYDMKHSATGPINE

SEQ ID NO: 288 - protein, 424 aa, Serratia marcescens
MFRQEAIDNQKMKWRGRALLLPGIPFWFTAGLCLFFLIAFLTFTIAGTYTRRVNVTGEISTYPRAANVYSTVQGIVVKQFVTEGQVIAVGAPIYQIDVSKSTRSGVLSDNQRRDIDGQLARIAQIINRLESSKQATLIMLEQQKAQYTAAFTRSTDILKRAQEGIRIMKENMENYRHYQTKGLINKDQLTNQVALYYQQQNNLLGLSGQNEQNALQITALESQIHIQAAEFDNQIYQMELQRYELQKERQNIDATGDIIVRALAGGRIDSLSVTVGQMVNIGDSLLQIIPQNIDRYALVLWVPNDAIPYIAAGDKVNVRYDAFPAEKFGQFAGTVSVISKAPASPQEMLTYQGAPKAALTAAVPYYKVIVIPEKQSIAYDGKRLSLENGMKAQSTLFLEKRKMYQWMLSPFYDMKHSATGPVNE

SEQ ID NO: 289 - protein, 424 aa, Serratia marcescens
MFRQEAIDNQKMKWRGRALLLPGIPFWLTAGLCLFFLIAFLTFAIAGTYTRRVNVTGEISTYPRAANVYSAVQGVVVKQFVTEGQVIAAGAPIYQIDVSKSTRSGVVSDNQQRNIDGQLARIAQIISRLESSKQATLTMLEKQKAQYIAAFTRSTDILKRAQEGIRIMKENMENYRHYQTKGLITKDQLTNQVALYYQQQNNLLGLSGQNEQNALQISALESQIHIQAANFDNQIYQMELQRHELQKERLNIDAGGAIIVRALADGRIDSLSVTVGQMVNVGDSLLQVIPHNIDHYALVLWVPNDAIPYIAAGDKVNVRYDAFPAEKFGQFAGTVAVISKAPASPQEMLTYQGAPKAALTAAVPYYKVIVIPEKQFIAYDGKRLSLENGMKAQSTLFLEKRKIYQWMLSPFYDMKHSATGPVNE

SEQ ID NO: 290 - protein, 424 aa, Serratia plymuthica
MFRQEAIDNQKMKWRGRAILLPGIPFWAMAGLCFFFIVAFLSFIIAGTYTRRVNVTGEISTYPRAANVYSGVQGVVVKQFVTEGQEIKSGDPIYQIDVSKSTRSGVVSDNKRQDIDNQLERITQIISRLESNKKATLDMLEKQRAQYTAAFQRSTDILRRAQEGIHIMKENMENYRRYQTRGLINKDQLSDQVALYYQQQNNLLGLSSQNEQNALQITALESQIHTQAAEFDNQIYQMELQRYDLQKEMLNIDADGTIIVRVLADGRVDSLSVTVGQMVNVGDSLLQIIPHKIDHYALVLWVPNDAIPYIATGDKVNVRYDAFPAEKFGQFAGTVSVISKAPASPQEMLTYQGAPKAALTTAVPYYKVIVRPEKQVIVYNRKRLSLENGMKAQSTLFLEKRSIYQWMLSPFYDMKHSATGPVNE

SEQ ID NO: 291 - protein, 424 aa, Serratia surfactantfaciens
MFRQEAIGNQKLKWRGRALLLPGIPAWFTAGLCLFFLIAFLTFVLAGTYTRRVNVTGEISTYPRAANVYSTVQGVVIKQFVTEGQRIAAGAAIYQIDVSKSTRSGVVSDNQQRDIDGQLARIAQIISRLESSKQATLTMLEKQKAQYTAAFTRSTDILQRAQEGIRIMKENMENYRHYQTKGLINKDQLTNQVALYYQQQNNLLGLSGQNEQNALQITALESQIHTQAAEFDNQIYQMELQRYELQKERQNIDATGDIIVRALAGGRIDSLSVTVGQMVNVGDSLLQVLPHDIDRYALVLWVPNDAIPYIAAGDRVNVRYDAFPAEKFGQFAGTVSVISKAPASPQEMLTYQGAPKAALTAAVPYYKVIVIPEKQTIAYDGKRLSLENGMKAQSTLFLEKRLVYQWMLSPFYDMKHSATGPVNE

SEQ ID NO: 292 - protein, 430 aa, Yersinia pseudotuberculosis
MSSNQIFRRDAVEYIRTKWLGKALLTSGYSTTFIAALCAIFLVLLITLIIYGTYTRRINVNGEVISQPHPINIFSPQQGFITKKWVEVGDIVRKGQHLYQIDVSRTTFSGNVSLNSLEAINNQLSQIDSIINNTQKNKELTLLNLRQQLAQYQKAHKKSQELVDNAGKGMDDMRRTMASYGTYQRQGLITKDQLTNQRSLFYQQQNAFQSLNTQLIQESLQIAKLESEISTRASDFDNDISQYLFQKGDLKRQLAEVDASGMLLINSPSDGKIENMSVTQGQMVNVNDSLVQLTPSDNPYYCLVLWVPNNSVPYINTGDKVNIRYDAFPFEKFGQFPGRIISISNVPVSQQEIASYNIAPRLPNGGLIEPYYKVIVALDDIHFRYQSKPLMLSNGLKANVTLFLEKRPLYQWMLSPFYDIKKSVTGPVNE

SEQ ID NO: 293 - protein, 425 aa, Escherichia coli
MFRQEATVYRKMKWCGRAVLLPGIPPWLVISVSVFFFISFIVFVMEGTYTRRINVEGEITTWPRPVNVYSGVQGVVIKQFVTEGQRIKKGDPIYLIDVSKSTSSGVVGDNKRRDIEKQLSRIGDIISRLEENKKTTLQTLEKQRIQYYSAFERSTEILRRAEEGVKIMKSNMDNYKQYQTKGLINKDQLTNQIALYYQQQNNILSLSTQNEQNLLQVTSLESQMQTLAAEFDNRIYQVELQRYELQKELVDTDAGRDIIIRALSDGKIDSLSVTPGQMVSVGDSLLQIIPEEIKNYHLIVWVPNNAIPYISVGDNVNVRYEAFPPEKFGQFTAKIMLISRTPASAQEMQTYPGAPRNNTGVSVPYYKIVLNPEQQTIEYGEKKMPLENGMKAQSTLFLEKRKIYQWMLSPFYNMKYSAVGPVNEQ

SEQ ID NO: 294 - protein, 428 aa, Klebsiella sp.
MKNELYRKEAIEYKRNYWKGKALLLAGMPSWLIGSLSFLFLICLILTLIFCNYTQRIDVKGEVITLPHSINVFAPQQGFVIKKHVAVGDIVEKGTPLYEIDVSRSTINGNVNETMTAVITEKIKNANEIINKILKNKTETLDALNIQIKQYELTLQETDKMLKNAGDGLKKMKDNLSSYDKYLKEGLITKDQYNYQHSLYFQQQSTYQSLVSQKMQIASQLTQLNSDKIIKGADFDNQISTQKNQINDYKNQRVESTANGNVVVKATAAGKIESVSVTAGQMVDKGSSLAQIKPTGNVEYYLILWLPNNSIPYINPGDTVNIRYDAFPSDKFGQFPGKVVSISSVPASRQEMSEYTNAITNPNQQELALYKATIKIKKTNFIYRNKVLKLSNGLKAQAIVFLEERPLYMWMFTPFYKINQSVSGPINE

SEQ ID NO: 295 - protein, 425 aa, Klebsiella aerogenes
MFRKEALENRDKTWCGKAVLLPGISSRLVIITSALFFVCFIVLISTGTYTRRISVQGEVTTWPRPVNVYSGVQGFVATQFVREGDPVKKGDPVYQIDVSKSTRRGVVNETQRQDIENQLKRIAEIIARLRESKETTLRMLEQQKIQYSASFERSAGIVRRAEEGIKLMKNNMDNYRRYQQQGLINKDQLTSLVALYYQQHNSLLSLSAQHEQNALQLTNLESQIQTQAAEFDNRVYQMELQRYELQKERINTDAGGDIIIRASSDGKIDSLSVTPGQMVSEGDSLLQIIPEKIQRYYLVLWVPNEAVPYISVGDRVNIRYEAFPAEKFGQFSAKVTLISRTPASYREMQTYTTAPINSTAGAAAYYKVVISPEKQAVSYAGKRLPLENGMKAQSTLFLEKRKIYQWMLSPFYDMKRSVTGPIHEP

SEQ ID NO: 296 - protein, 427 aa, Kosakonia oryzae
MGQKLFRQEAVDHQRSYWKGKVILFKGVSPFIISACCIAFMAVLIAMLCFFKFTQRIDVVGEVITLPHPLNISSPQPGFISQQFIQVGDTVKKGDVLFELDVSKSTNAGNVSDTNISMIKAKITNAEEIINKLKANKDDFIATTQRQIQRYEYSLTETDGLLATAKQGLTKMESALQNYNQYLKKGLINKDQYNNQHSLYVQQQNAFQSLSTQKMQLELQLTQSRSDITVKSAELDNQIAAQYNQLSDFQSQLIETNANGKILVKSTIGGRVESVAVTPGQMVDAGSSLAQIKQLTDVKYYLLLWLPDNSLPYVKIGDTVNIRYDAFPSDKFGQFSGKIKSISSMPATRRELAEYSIGQSNTNSAQTLYKAIIDIKQKEFIYKDKKLELSNGLKAKSLVFMEERPLYQWLFSPVYKVAQSLTGPNNE

SEQ ID NO: 297 - protein, 427 aa, Kosakonia radicincitans
MGQKLFRQEAVDHQRSYWKGKVILFKGVSPFIISACCIAFMAILIVMLCFFKFTQRIDVVGEVITLPHPLNISSPQMGFISQQFIQVGDTVKKGDMLFELDVSKSTNAGNVSDTNISMIKAKIANAEEIINKLKVNKDDFIATTKRQIQRYERSLAETDGLLTTAKQGLTKMEGALQNYNQYLKKGLINKDQYNNQHSLYVQQQNAFQSLSTQKMQLELQLTQSRSDITVKSAELDNQIASQYNQLSDFQSQLIETNANGKILVKSTIAGRVESIAVTPGQMVDAGSSLAQIKQLTDIKYYLLLWLPDNSLPYVKIGDTVNIRYDAFPSDKFGQFSGKIKSISSIPATRRELSEYSAGQTNTNSQLTLYKAIIDIKENEFIYKNKKLALSNGLKAKSIVFMEERPFYQWLFSPAYKVAQSLTGPKNE

SEQ ID NO: 298 - protein, 428 aa, Pantoea sp.
MLFRKEVSEHLQSRYAGPILLITGWPVWITLCITLLFLAALLLFLWQGSYTRRTSVSGEVITWPHTINLFAPEQGVISRLLVSPGQQVSAGTPLYALDISRVSASGNLSATTRALLKKQQQQMGEMLHQLEQNRRATLASIQQQLDQLRSAREKTQQMVDSATQGLDVMRSSMNDYNRFARRGLITSDQQNNQRYLFYQQLSVWHSLNSQRVQQDMQIAELLSQQVTRAADFDSQLAQYNIRQADIERQLAEADAGSERLIAAPSAGRISSLSVTAGEMVSDGDSLAQLVPAGDDIPSLVIWLPNDSIPYVKPGDVINVSYAAYPAEKYGQFPGKVLSISSAPVPLRELNNYGSAPRSPAGQITGAWFKANVALNHQGLRWQGKDLPLASGMQLQATVFLEKRPLWQWMLSPYYFLKNSITGPVSDQR

SEQ ID NO: 299 - protein, 448 aa, Rahnella sp.
MCCYLSKILFLQPVFLEHLNMLFRKEVNEHQQSHWAGKALLLAGWPLWIVTTLTALFLFALLVFLIFANYTRRINVSGEIITQPHSINLFSPEQGVVTKLYVDTGKPVKKGQPLYEIDVSRVTQAGNVSTTTLAAIKKQREQIDSIIAQLEHNKQETLQNLQQQLDQYQKAHEVSQSMVDSAQEGLDAMKKSMQNYGAYQKKGLINTDQLNNQRYLFYQQQTSFQSLNTQAIQEALQITNLRSELVTRAAEFDNQISQYGYQRNDLERQLAQADAKGSLLITAPTSGKISSLSVTPGQMVNAGDSLAQLVPASNSPFFLVAWLPNESVPYVKAGEHINIRYEAYPFEKYGQFPGKVESISSAPVSEQELNSYASAPRTANGTISGPYYKVIVSLDKKAMNWHGETLNLSSGMKAESTLFLEKRPLYQWMLSPYYSMKKSVVGPINESR

SEQ ID NO: 300 - protein, 448 aa, Rahnella sp.
MCCYLSKILFLQPVFLERLNMLFRKEVNEHQQSHWAGKALLLSGWPLWIVTTLTALFLFALLIFLIFANYTRRINVSGEIITQPHSINLFSPEQGVVTKLYVDTGKPVKKGQPLYEIDVSRVTQAGNVSTTTLAAIKKQREQIDSIIAQLEHNKQETLQNLQQQLDQYQKAHEVSQSMVDSAQEGLDAMKKSMQNYGAYQKKGLINTDQLNNQRYLFYQQQTSFQSLNTQAIQEALQITNLRSELVTRAAEFDNQISQYGYQRNDLERQLAQADAKGSLLITAPTSGKISSLSVTPGQMVNAGDSLAQLVPASNSPFFLVAWLPNESVPYVKAGEHINIRYEAYPFEKYGQFPGKVESISSAPVSEQELNSYASAPRTANGTISGPYYKVIVSLDKKAMNWHGETLNLSSGMKAESTLFLEKRPLYQWMLSPYYSMKKSVVGPINESR

SEQ ID NO: 301 - protein, 427 aa, Raoultella sp.
MGKRLFRREAIEHQRSYWKGRVILLRGVSPLIISACCVAFLVMIIAMLCLFKYTKRIDVVGEVITLPHPLNISSPQPGSVSRQSIQVGDVVKKGEVLFELDVSKSTDGGNVSDTNVAMVKAKIVNAEDIIAKLKVNKDGFIETTHRQILRYEHSLNETEKMLTTAQKGLTHIEKSLQNYSDYLKRGLINKDQYNNQHSLFVQQQNAFQSLSSQKMQLELQLTQTRSDLTVKTAELDNQITSQYSQLRDLQTQLIENSANGKIVVKSTIDGRVESIAVTRGQMVEAGSSLAQVKQLTGIEYYLLLWLPDNSLPYVKIGDIVNIRYDAFPSDKFGQFSGKVLSISSMPATRRELAEYSNNQMSNAPAPTLYKAIINIKDKEFIYKDRRLVLANGLKAKSIVFTEERPLYQWLFSPVYKVVQSLKGPGDE

SEQ ID NO: 302 - protein, 431 aa, Serratia sp.
MSDNKSRNIFRQEAVNYQRQRWTGRALLISGASSHVIAATCFTFILILAGALYFCEYTRRVDVEGEVISIPHSTNVFSSQYGYVSQAFHKAGDVVEKDEPLYVIDVSRSTTSGNVSHIAASEISKQISNLDSIISKLNKNKTAMLDNLRKQINSYENAYKETEKLNLSAKEGMDKMRDSLKNYEHYLTKGLITKDQLNYQRSLFQQQQSSYQALYSQKIQQNIQLSQLRSDLLIRAADYDNQISQSESQRSDLQRQLAESNAGGQVIIKAPLKGKIESLSVTAGQVVESGSSLAQIKPIDNIKYYLVLWLPNNSLPYVKIGDGINIRYEAFPSDKFGQFPGKIESISSVPASPQEMSEYSSGNKRERNAYYKVLASISDTQFSDKGKYLEISSGLQARVIVFLDKKPLYQWAVAPLYDIKNSVVGRVDEGQ

SEQ ID NO: 303 - protein, 431 aa, Serratia sp.
MSENKSRNIFRQEAVNYQRQRWTGRALLISGASSYVIAVTCFIFILILAGALYFCEYTRRVDVEGEVISIPHSTNVFSSQYGYVNRSFHQAGDVVEKDEPLYVIDVSRSTASGNVSHIAASEINKQIGNLDSIISKLNKNKDAMLDNLRKQINSYELAYKETEKLNLNARDGMEKMRESLKNYEHYLTKGLITKDQLNYQRSLFQQQQSSYQALNSQKIQQNIQLSQLRSDLLIRAADYDNQISQSESQRSELQRQLAESNAGDQVIIKAPLKGKIESLSVTAGQVVESGSSLAQIKPIENIKYYLVLWLPNNSLPYVKIGDGINIRYEAFPSDKFGQFPGRIESISSVPASPQEMSEYSSGNKRERNAYYKVLASISDTQFSDKGKYLEISSGLQARVIVFLDKKPLYQWAVAPLYDIKNSVVGRVDEGS

SEQ ID NO: 304 - protein, 417 aa, Escherichia coli
MNIFRSEAIEHHNDTEYGDIILPTSFSLSVCATVTLFIMLSLTVFIYYGSYTRKAHLTGIVMPSSGLVKIIPQYAGYVTQLTVSEGEHVTAGTQLYHISGEHYNGNGTGTLATMSISLKTQYIMLASQQSFESRDNSQQQEAIRQRMISLEPQIRSAEQRLQLAERQAELAISVMERYKKLAGTHYVSDIEFQQKQIDVSAAQQNVEDQRQGLLQLHTAMDTAKDELNHLIVQGKSRKAELDRQLQVLKQQQDELAGQEKFTLRAPVSGTIAAVLIKQGQSVKASEPVMTLIPDNAHLQIELYATSQKAGFIRPGQRVSLKFSAFPYQKFGIQYGTIRKISHTTLAPSDLLPVSPVTWKENEGHYRVIVEPENTFIFAYGKKEPLRPGMTLEGDVNLDTRHLWEWLTEPLWSMKGNL

SEQ ID NO: 305 - protein, 417 aa, Enterobacteriaceae sp.
MNIFRSEAIEHHNDTEYGDIILPTSFSLSVCATVTLFIMLSLTVFIYYGSYTRKAHLTGIVMPSSGLVKIIPQYAGYVTQLTVSEGEHVTAGTQLYHISGEHYNGNGTGTLATMSISLKTQSIMLASQQSFESRDNSQQQEAIRQRMVSLEPQIRSAEQRLQLAERQAELAISVMERYKKLAGTHYVSDIEFQQKQIDVSAAQQNVEDQRQGLLQLHTAMDTAKDELNHLIVQGKSRKAELDRQLQVLKQQQDELAGQEKFTLRAPVSGTIAAVLIKQGQSVKASEPVMTLIPDNAHLQIELYATSQKAGFIRPGQRVSLKFSAFPYQKFGIQYGTIREISHTTLAPSDLLPVSPVTWKENEGHYRVIVEPENTFIFAYGKKEPLRPGMTLEGDVNLDTRHLWEWLTEPLWSMKGNL

SEQ ID NO: 306 - protein, 417 aa, Enterobacteriaceae sp.; identical to SEQ ID NO: 304

SEQ ID NO: 307 - protein, 417 aa, Salmonella enterica
MNIFRSEAIEHHNDTEYGDIILPTSFSLSVCATVTLFIMLSLTVFIYYGSYTRKAHLTGIVMPSSGLVKIIPQYAGYVTQLTVSEGEHVTAGTQLYHISGEHYNGNGTGTLATMSISLKTQSIMLASQQSFESRDNSQQQEAIRQRMVSLEPQIRSAEQRLQLAERQAELAISVMERYKKLAGTHYVSDIEFQQKQIDVSAAQQNVEDQRQGLLQLHTAMDTAKDELNHLIVQGKSRKAELDRQLQVLKQQQDELAGQEKFTLRAPVSGTIAAVLIKQGQSVKASEPVMTLIPDNAHLQIELYATSQKAGFIRPGQRVSLKFSAFPYQKFGIQYGTIREISHTTLVPSDLLPVSPVTWKENEGHYRVIVEPENTFIFAYGKKEPLRPGMTLEGDVNLDTRHLWEWLTEPLWSMKGNL

SEQ ID NO: 308 - protein, 417 aa, Escherichia coli
MSIFREEALEHNSDTEYGDIVLPASFGMSVCAVATLLIFLSVSLFVYYGSYTRKAHLTGIVMPSSGLVKITPQYAGYVTRQTVSEGQHVAAGEPVYHISGEHYNGQGTGTLAAMSMSLKTQYAMLSSQQTLELRDNSQQQQAILQRIASLKPQIKSAELRLSLAGHQATLAVSVMERYKKLAGTHYVSDIEYQQKQIDVSTSLQNVEDQRQGLLQLHTAMEAAEEDLNHLIVQGESRKAELDRQLQGIRQQQDELAGQENFTLTSPVSGTVAAVLIRQGQSVRASEPVMTLVPDNARLQIELYATSQNAGFIQPGQRVALRFAAFPYQKFGVQYGTIREISRTTLTPSDLLSVSPVTWKENEGHYRVIVEPENTFILAYGKQEPLRPGMTLEGDVSLDTRHLWEWLTEPLWSLKGKL

SEQ ID NO: 309 - protein, 417 aa, Citrobacter amalonaticus
MKLFRKEVIENLNSTTYGGVIIPTSFSLLLSTIATIALIVLISIFLGFGEYTRKARLTGIVMPSSGLIKITPQYSGYVTKLTIQEGEHVQKGQALYHISGEHYGSKGTGTLAAMNLSLQTQYSMLSSQKMLEQRDNNQQQQSIQQHITLIQLQIKSGEQRLLLAKHQADLSSLVVRRYQKLINQKYVSEIEYQLKQIESASAKENVENQRQLLLQLRASLDNARDELAHLIVQGESRNAELSRQLQVIKQQQFELASQESFTLTAPVAGTVAAILIRQGQSVKAQEPVMSLVPENSDLQVELYATSQNAGFIQPGQKVALRYTAFPYQKFGVQYGTILEISRITLAPTDLLSISPMIWKVNEGHYRVIVKPELSHILAYGKYEPLRPGMTLEGDVNLDTRYLWEWLTEPLWSLKGKI

SEQ ID NO: 310 - protein, 417 aa, Enterobacter sp.
MNIFREEALNHQTDSDYGEIVLPASFGLSVSALTTLFIFLCLILFILYGSYTRKAHLSGIVMPSSGLVKITPQYAGYVTKLTASEGQHIIAGETLYHISGEHFNEQGAGTLAAIRLSLQTQFTMLTSQQTLELRDNSQQQHAIQQRIVSLKPQVKSAEKRLSLAVHQAALAVSVMERYKKLADTHYVSDIEYQQKQIDVSTSQQNVEDQRQGLLQLHTAMEAAKDDLDHLIVQGESRKAEMDRQLQVIRQQQDELAGKENFTLSSPVSGTVAAVLVRQGQSVRASEPVMTLIPDNARLQIELYATSQNAGFIQAGQRVALRFSAFPYQKFGVQYGTIREISHTTLTSSDLLSVSPVTWKENEGHYRVIVEPENTFILAYGKQEALRPGMVLEGDVSLDTRNLWEWLTEPLWSLKGKL

SEQ ID NO: 311 - protein, 417 aa, Enterobacter cloacae
MNIFREEALNHQTDSDYGEIVLPASFGLSVSALTTLFIFLCLILFILYGSYTRKAHLSGIVMPSSGLVKITPQYAGYVTKLTASEGQHIIAGETLYHISGEHFNEQGAGTLAAIRLSLQTQFTMLTSQQTLELRDNSQQQHAIQQRIVSLKPQVESAEKRLSLAVHQAALAVSVMERYKKLADTHYVSDIEYQQKQIDVSTSQQNVEDQRQGLLQLHTAMEAAKDDLDHLIVQGESRKAEMDRQLQVIRQQQDELAGKENFTLTSPVSGTVAAVLVRQGQSVRASEPVMTLIPDNARLQIELYATSQNAGFIQAGQRVALRFSAFPYQKFGVQYGTIREISHTTLTSSDLLSVSPVTWKENEGHYRVIVEPENTFILAYGKQEALRPGMVLEGDVSLDTRNLWEWLTEPLWSLKGKL

SEQ ID NO: 312 - protein, 417 aa, Klebsiella variicola
MGVFRSEALEYHSDTEYGDVVIPASLSMTACAVATLFIFICVVLFVYYGSYTRKAHLTGIVMPSSGLVKITPQYAGYITQLTVSEGQHVIAGEPLYHINGEHFNEQGAGTLAAMNLSLRTQYAMLSSQETLELRDNQQQQAAIKQRIASLQLQVKSAEQRLQMAERQVGLTTSVMGSYKKLAGTHYVSDIEYQQKQIEVSTAQQNVEDQRQGLLQLRTAIEAAKDDLNHLIILGGSRKAELDRQLQEIRQQQEELAGQENITLTAPVSGTVAAVLVRQGQSVRALEPVMTVVPDNASLQIELYAASQNAGFIRPGQRVALRFAAFPYQKFGVQYGTIREISRTTLTPSDLLSVSPVTWKENEGRYRVIVRPENPFILAYGKKEPLRPGMTLEGDVNLDTRPLWEWLTEPLWSLKGKL

SEQ ID NO: 313 - protein, 418 aa, Pseudomonas syringae
MLFRPEALDAQKRSTLGTVLLVSPLSMRLAAAVALLFCISMGLFLTFATYTKRTAASGVLLPETGLIRIYSPQAGVITRLDIVEGQHVTDGAVLMALSSDTQSGEAGGAQRAISLSIKRRQESLSQEIDETLALHLQELEGKKRQVAALETEQQKILAQIDLTRQRLALTKGLAERYANLQRQDYVSRDQLQEKQDAVLDMRLRGEELNRSLLTVRAESARLRAELVELPFNQSKQLADLRRRLAENQDRLIESETKREIFITAPTLGEATAIAVSNGSRVDSTRPLLSIVPADAKLHAELYLPSRSVGFVRTGDTVMLRYQAYPYQRFGLQPGTVSSISRTALPADEVMTLGNVSEPMREQGPFYRVTVALASQTIDGQGMHERLRSGMQLDADIMQERLPLYEWMLEPLRGIGKRL

SEQ ID NO: 314 - protein, 418 aa, Pseudomonas syringae
MLFRSEALDAQKRSTLGTVLLVSPLSMRLAAAVALLFCISMGLFLTFATYTKRTAASGVLLPETGLIRIYSPQAGVITRLDIVEGQHVTDGAVLMALSSDTQSGEAGGAQRAISLSIKRRQESLAQEIDETLALHLQELEGKKRQVAALETEQQKILAQIDLTRQRLALTKGLAERYANLQRQDYVSRDQLQEKQDAVLDMRLRSEELNRSLLTVRAESTRLRAELVELPFNQSKQLADLRRRLAENQDRLIESETKREIFITAPTLGEATAIAVSNGSRVDSTRPLLSIVPADAKLHAELYLPSRSVGFVRTGDTVMLRYQAYPYQRFGLQPGTVSSISRTALPADEVMTLGNVSEPMREQGPFYRVTVALASQTIDGQGMHERLRSGMQLDADIMQERLPLYEWMLEPLRGIGKRL

SEQ ID NO: 315 - protein, 422 aa, Serratia inhibens
MTGRPPQLYREGYFTAQATKAEGEVIIPPSLSLTAYAWASGALVLAILLFLMLGEYTRKTRLEGIVMPSSGLIKVVARNAGQVTERLVAEGEKVKAGQPLYRLSGEHFNGQGVGALASVAASLDQQYRMLEHQREQERATSALRQTGVRQRIAQLDDELTSLGSALTLVQQQAAFSHSVMARYRKLIAKGYVSEIEFQQKQMELSTMREKVEVQRQAQLRLKRELAAAGSELQTLQQQQQGRDAELERQLQGIDQQKIELGAQRDNTLTAPVDGDVAVVLARAGQTVKPNDPLLMMVADGAQLQVELFAPSKAVGFIKPSQRVGLRFASFPYEKFGVQYGSTRTITHASLSAGDALLQNPLVWKENEGHYRVIVALDKTTITAYGRQEPLRVGMAVSADIELDSRRLYEWLLEPLWSLQGRM

SEQ ID NO: 316 - protein, 422 aa, Serratia liquefaciens
MSERSPQIYREGYFAAQVNKAEGEVIIPASLSLTAYAWASILLVLAIGLFLILGEYTRKARLDGVVMPSSGLIKVVARSAGQVTERLVTEGATVKAGQALYRLSGEYFNGQGIATLASVSDSLKRQYHMLERQRAQELATRILQQTGVRQRMTQLQDELASAEAAMVEVKQQAELSHSLMARYRKLIGKGYVSELEFQQQKMTLSTAREKVEMQRQAQLRLKRELAAAASELRTLAQQQQGRDAELERQLQGIRQQEIELVAQRDHTLTAPVDGHVVVVLARAGQTVKQNDPLLMMVADGAELQVELFAPSKAVGFIKPHQRVGLRFASFPYEKFGVQYGATRAITDASLSANDVMQQNPMTWKENEGHYRVIVALEKTTITAYGRQEPLRVGMTVSADVELDRRRLYEWLLEPLWSLQGRI

SEQ ID NO: 317 - protein, 422 aa, Serratia plymuthica
MTGRPPQLYREGYFAAQATKAEGEVIIPPSLSLTAYAWASGALVLAILLFLMLGEYTRKARLEGVVMPSSGLIKVVARNAGQVTERLVAEGAAVKAGQPLYRLSGEHFNGKGVGALASVAASLDQQYRMLEHQREQEQVTSALRQTGVRQRIAQLDDELTSAGSALVLAQQQETLSHSVMARYRKLIAKGYVSEIEFQQKQMELSTAREKVESQRQAQLRLKREWAAAGSELQTLQQQQQGRDAELERQLQSIDQQKIELNAQRETTLTAPVDGHIAVVLARAGQTVKPNDPLLMMVADGAQLQVELFAPSKAVGFIKPSQRVGLRFASFPYEKFGVQYGSTRAITHASLSAGDAMLQNPLVWKENEGHYRVVVALDKTTITAYGRQEPLRVGMAVSADIELDSRRLYEWLLEPLWSLQGRM

SEQ ID NO: 318 - protein, 428 aa, Vibrio casei
MVARAKHLYRPEYFDAQKSDNQGSILIDASLNQHLFVAASMFVLMGIIFFVVFAEYTRRGTLNGVVSPTGGIVKVQANDEGYAEKVFVTEGQKILSGEPLYEIKTERYDEFGQGVKKRIVTAIENQITLLNERRNKEIEKSQHQLQVIDYDQVRLSEEASILEVVVDLSEQELKLAQSLVDKQQVLVNKQFISKIEFQRQRLELITLKTKVQTQRLSLQRLLREKAKLKETKETLQLELDIALQEIDRQLQQIDQSKIEYFYQTDTQVVSPVDGVVASIFVKEGHSVNKGQPLLVVIPKGEEPAIVELYASSRSIGFLQKGQGVRLRFDAFPYEKFGVQQGVISSISKSAVSAEMLPNSPLIQSQLSRANGGMGLYQVRVKLDKPTITVYGEEQLFVPGMTLTADIELDTRKVYEWLLEPLYTIQGRI

SEQ ID NO: 319 - protein, 418 aa, Vibrio harveyi
MRQFYREEYLTIKTKTPEARILIRSSYSQSAMILVCIIIFLAFFLLGFFGEYTRKASLQGVVNPYGGMVNVQSGSTGYIEELLVKEGEVVVAGQAIYKVNTERHNESGEPIKPLLNQSYNAQTKIIRDRIVQEQKRVGIELNSLNDDIDNLNVNIQNTQRIVSLSRQELELRQALFADKKKLLKLGHISEVEYANEQLKVNEVASRYESNLLNYNAKNRELAALKHAVKSTEINSAITIKDLDRQLEVVEQERSEFLYQTDTRVVAPITGVVASVLVKEGHSVIAGQTLLVIVPEGEVFVEVYAPSRDIGFINLGQKVKLRFEAFPYQKFGVQGGIVESVSRTAVSPDVITNQRLIKADKVEGLYQVRITLNKQTITAYGREELLKSGMAVTADVEVDTRKLYEWVLEPMYSIKGRLE

SEQ ID NO: 320 - protein, 413 aa, Vibrio cholerae
MNLFREEVFQAKKNRLFGDVVINQPAFLYVIITAVFVLFIVLSIYISQSSYSRKETVKGYLIPEKGLMKVFLNRSGVVGDLLVEEGSKVNAGDGLAKIINSQSLSSGVELSDILVDELEKQKVTLSEELRVIYKIQENDINQISLKLAQLEDSLNVIKKIKLTVQNKLELKERLFTNNKELYERGYISKINLFDSHEEYLNIRESMDGLDREILDLVAKIADFEVKKNSIPNEVELKRISIERRISEINMQILELKNKYEFIVKAPESGTVTAIQAYTGTNINNNVPLLSIIPEDSILEIEMLLPTRSAGFIQIGDPVRIRFDAFPYQKFGSIKGEVTKIDKALLLPTDQMLPIRLEEASYKVSAKLEKQEIVAYDKSFSLKVGMIAEADIILENRSLLDWLLDPIYALQGKL

SEQ ID NO: 321 - protein, 415 aa, Methylomonas sp.
MKHELFRQQALDFKKDKPLGEVIHVESLSFSFLTALAVSSALVLMAFAFWGEYTRKSHVNGYLSPSMGLIKVYAPQAGTLIEKHIREGQAVKAGDTLFVLSTESSSRETPQAQAAAIEQLKQRRESLETELVKQEQIDQIQYRSVLDRLHGMQRELQQVNSEIDTQQQRLAGAAATSQRYQQLLTTKFVSAVQAQQKQDEWLDHQAKLQALQRVQMTLQRDIRSAQLEADSSQMKFKNQRAAIERNISTLAQNITESESRRNIVITAPHDGTVTTILAEQGQNATTANPLLSILPLGAKLQAQLLVPSSAVGFVEQGQTVSVRYQAFPYQRFGSHQGRIVEIAKTLITPKEADLPVTLQEPVYRVTVEIDKQAVQAYRQDIPLQSGMLLDADIWLDHRRLIEWVFDPLYSILGRV

SEQ ID NO: 322 - protein, 415 aa, Shewanella oneidensis
MSDLFRQQVVNEQKQRLYGDISLAQPLSIYTISIAILLIVTAIILFLYFSHYARKETVRGYLVPDKGVIKTYANRIGNVDILHVKEGSDVNAGDPLVTVIIRSSMASGFELSETLISELKQQQSILNQELDNNIELNAAETLRLKKRLSDLSESMKVLSRQKQLLADKLNIQVAQKKQHDKLYKDGYLSELDYQLQLSKLIEVKQEVENLESNKISIDRELNQTHAELVSLPFQFNLKQSDVHKRQSDIQRQLNEAENSYTFVIRAQESGTVAAVSVVEGEFIASNRPLMSIIPKGSSLVAELLLPTRSAGFVKQGDEARLRFDAFPYQRFGFLHSEVLRVDKALLLDGEADLPVKLSEPVYRIKTTLSAQDMQAYGEAFPLKSGMLLEADIVLDRRTLLDWLLDPIYSLRGRVS

SEQ ID NO: 323 - protein, 415 aa, Shewanella putrefaciens
MATLFRQQVVDEQKQRLYGEISLAQPLSIYTVSVSIFFTVLVVALFLYFSHYARKETVRGYLVPDKGVIKTYANRNGNVEVLHVKEGANVNAGDPLVTVIIRSSMTSGIELSEVLINELKHQQQILNEEIKNNSALNLSETKRLEKRLSDLIESMAVVSRQKALLDDKLTIQINQNKQHDKLYKDGYLSELDYQFQLSKLIEVKQELENVESNRVSINSELNQTRSELASLPFQFNLKQSDVHKRQSDIQKQLNEAENSYRFVVRAQESGTVASVSVVEGEFIANNRPLMSIIPKGSSLVAELLLPTRSAGFVKQGDEARLRFDAFPYQRFGFLHSEVLRVDKALLLDGEADLPVKLTEPVYRIRTTLSAQDMQAYGEAFPLRSGMLLEADIVLDKRTLLDWLLDPIYSLRGRVS

SEQ ID NO: 324 - protein, 392 aa, Vibrio harveyi
MTQPFSFYFKVAFVFMSFFLIFIYLNQYSYTRKESVKGYLTPKKGLVKVYLNREGIIEELMVSEGKEVKIGDGLAKIKNSQSLSSGEELSESISNELKKQKIVLTNEISILKEIKYKDNLKLENQLIKLYKSYSSAKKLRETSLKKLKIKKDNYLINRDLKDKGFLSRNEFLVSLEEYLNAQENVDRLDQNLVSIELEISIKESEKNSLPENLQLKLTTIERQISSINTQLYELNNQYEFIKKAPESGIVTAIQSYVGSNVNLKTPLLSIIPKDSPLELEMLLPTRSSGFVQVGDHVNIRFDAFPYQKFGMVKGTIISVDKALLLPSDKALPFAVNEAMYQVRAELDNQTVLAYGKSYPLKVGMIADADIILEKRSLLDWLLEPLYSVKGRF

SEQ ID NO: 325 - protein, 413 aa, Escherichia coli
MKWQGRAILLPGIPLWLIMLGSIVFITAFLMFIIVGTYSRRVNVSGEVTTWPRAVNIYSGVQGFVVRQFVHEGQLIKKGDPVYLIDVSKSTRSGIVTDNHRRDIENQLVRVDNIISRLEESKKITLDTLEKQRLQYTDAFRRSSDIIQRAEEGIKIMKNNMENYRNYQAKGLINKDQLTNQVALYYQQQNNLLSLSGQNEQNALQITTLESQIQTQAADFDNRIYQMELQRYELQKELVNTDVEGEIIIRALTDGKVDSLSVTVGQMVNTGDSLLQVIPENIENYYLILWVPNDAVPYISAGDKVNIRYEAFPAEKFGQFSATVKTISRTPASTQEMLTYKGAPQNTPGASVPWYKVIAMPEKQIIRYDEKYLPLENGMKAESTLFLEKRRIYQWMLSPFYDMKHSATGPLND

SEQ ID NO: 326 - protein, 413 aa, Escherichia coli
MKWQGRAILLPGIPLWLIMLGSIVFITAFLMFIIVGTYSRRVNVSGEVTTWPRAVNIYSGVQGFVVRQFVHEGQLIKKGDPVYLIDISKSTRNGIVTDNHRRDIENQLVRVDNIISRLEESKKITLDTLEKQRLQYTDAFRRSSDIIQRAEEGIKIMKNNMENYRYYQSKGLINKDQLTNQVALYYQQQNNLLSLSGQNEQNALQITTLESQIQTQAADFDNRIYQMELQRLELQKELVNTDVEGEIIIRALSDGKVDSLSVTVGQMVNTGDSLLQVIPENIENYYLILWVPNDAVPYISAGDKVNIRYEAFPSEKFGQFSATVKTISRTPASTQEMLTYKGAPQNTPGASVPWYKVIATPEKQIIRYDEKYLPLENGMKAESTLFLEKRRIYQWMLSPFYDMKHSATGPIND

SEQ ID NO: 327 - protein, 430 aa, Pantoea agglomerans
MFRKEFIHSRSRKWKAKVILLPGISMWIYCFFSTVFLVILISFIVAGSYTRRISVSGEYMTWPQPVYIYSDVQGTVAEQKVKPGQSVKKNEILYTINVSRSTNGGVVSENRKAAIKGQIHRLEETITLLKSSKVVTLNTLASEEKQYQDALLKSSDIVNLAQDGILKLKKNVENYHDYQQRGLVNKDQVANQTAIYYQQQNNLLNLITQNQQNALQITTIESQILTQRSEYDSKIYQAEMQLFDLKKEIINENARTSVSVPSLTEGTVQSMNVSPGQNITQGESLSLIKPRHVESSQIIVFAPNNALPYLKTGSPVRISFAAFPYENFGFFSGKISSVSRVAAPSAEMKKYHFSGEDIKNSGSYYKITIAPEKSIIRYANSDIPLESGMEAKVTLYLEKRRLYQWVMGPLYALKNSLNEGISEEKNQIFY

SEQ ID NO: 328 - protein, 425 aa, Pantoea allii
MLYRKEVTEARRNYWSGKAVLISDQHGRYVMAFTLFFFTAFFILITSCSYTRRISVSGEVVSTPRAVTVFSPQQGFIISRAVKQGDHVDKGQPLYRIDVSRTTTSGVVSQRHHESIQKRITTLNQIAADISSNRALTLSMLAEEKSRYESALLRSSEIVTHAREGLKLMKENMDNYRQYQHQGLINRDQLINQTALYYQQQNDMLGIESQNEQNALQVMNLRSALQTQAANFDNQLNQIAIQKSALESDLTDADAGSDQIITSPVDGTVDTLSVTPGQMVTSGDSLLQIIPGDAHYRTLVLWVPDSAIPYLSAGEPVNIRYDAFPAEKFGQFPGKIASVAHSPASWQEMSTYPGAPLHSLTGPQTWYKILVNPASDHFTYKNKTIRAENGMKASVTLFLEERKLYQWILSPLYDIRDSATGAIHE

SEQ ID NO: 329 - protein, 506 aa, Escherichia coli
MIALNTASPQGMQMKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNTLLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 330 - protein, 439 aa, Aeromonas sp.
MKRTLLSVMVLLGVSAGAHAENLLDIYQQAQIKDPQLLQSKAVRDQAFEKINESRAPLLPQIDLGAGLNYLQNKNDTQSNTNAKGEVTLSQSLYRRSNWVNLDISEKQATQSDVAYNLEIQNLMVRTAKAYFDVLKAMDTLEFVRANKVAVERQLEQTQQRFEVGLTAITDVHEAEASRDQALADEINAENSLDNSYETLRELTGIDHRNLDVLNTDRFSPQKTAFGSDKWLELALDKNLQLHSARISKDIAKEQIDLAKTGHEPTLDLGAGLQTSNTDYKLDSMPGGNDNQANIGLTFKLPIYSGGATSSKVKQAQYNYVAASELLEKNFRSVQSTVRSSYNNVNASIGSVRAYSQSVVSADSALKATEAGYEVGTRTIVDVLDSTRKLYEAKQKLSEARYNYILNILSLKQAAGILEAQDLVEVNQGLMPATAKAKG

SEQ ID NO: 331 - protein, 441 aa, Aeromonas sp.
MKRTLLSVMVMLGVSGAAHAENLLDIYQQAQIKDTQLLESKAKRDQAFEKINESRAALLPQINLGAGLNYLQNKNDTMTNTGATGSVTLDQSIYRRSNWVNLDLTEKSATQSDVAYNLEYQNLMLRTAQAYFDVLKAMDTLEFVRANKTAVERQLEQTQQRFEVGLTAITDVHEAEAERDQALADEINAENTLDNSYESLRELTGIDHRQLDVLNTDRFSPQKTAFNSDKWLELALDKNLELHSARIGKDIAKEQIDLAKTGHEPTLDLGAGLNAGYNDYKDEIRNPESNSNQGNIGLNFKLPIYTGGATTSQVKQAQFNYVAASEQLERSFRSVQSTVRSSYNNVNASIGSVRAYSQSVISADSALKATEAGYEVGTRTIVDVLDSTRKLYQAKQKLSEARYNYILSILSLKQAAGILEQKDLEEVNQGLMPAAQVTKKS

SEQ ID NO: 332 - protein, 441 aa, Aeromonas caviae
MKRTLLSVMVMLGVSGAAHAENLLDIYQQAQIKDTQLLESKAKRDQAFEKINESRAALLPQINLGAGLNYLQNKNDTMTNTGATGSVTLDQSIYRRSNWVNLDLTEKSATQSDVTYNLEYQNLMLRTAQAYFDVLKAMDTLEFVRANKTAVERQLEQTQQRFEVGLTAITDVHEAEAERDQALADEINAENTLDNSYESLRELTGIDHRQLDVLNTDRFSPQKTAFNSDKWLELALDKNLELHSARIGKDIAKEQIDLAKTGHEPTLDLGAGLNAGYNDYKDEIRNPESNSNQGNIGLNFKLPIYTGGATTSQVKQAQFNYVAASEQLERSFRSVQSTVRSSYNNVNASIGSVRAYSQSVISADSALKATEAGYEVGTRTIVDVLDSTRKLYQAKQKLSEARYNYILSILSLKQAAGILEQKDLEEVNQGLMPAAQVTKKS

SEQ ID NO: 333 - protein, 430 aa, Aeromonas caviae
MFLPILALLSSYTHAEGLLDIYNLAKQKDPQLLESKAMRDQSFEKINEEQASFLPKINLGLSANYVDDNLDMQTGSATNSAINLEQSLYNSANWINLDLAAKNATLSDVSYVLEHQNLVLRTIQTYFNVLQAEDLLIYSQATKKAIQRQLAQTQQRFEVGLAAITDVHEAEAAFDQSFADEIYAENTLENSYEALRELTGLEHRKLNKLDTERFSPQKTLWKIDKWTDAAIEKNLALHQARIKKELAKMQIDLSKSGHKPLVDLTMGLSSTYNNYKDNTVNTDGYSNQGDIGIKFSLPLYTGGATASQVKQSSFDYVAASEQLEKSYRSVMSAVKSSYNNVNASIGAVRAYQQAVVSAESALKATEAGYEVGTRTIIDVLDANRKLYDAKTKLAISRYSYIVNVATLKLNSGSLGKKEIIDIDNGLVNLR

SEQ ID NO: 334 - protein, 441 aa, Aeromonas caviae
MKRTLLSVMVMLGVSGAAHAENLLDIYQQAQIKDTQLLESKAKRDQAFEKINESRAALLPQINLGAGLNYLQNKNDTMTNTGATGSVTLDQSIYRRSNWVNLDLTEKSATQSDVAYNLEYQNLMLRTAQAYFDVLKAMDTLEFVRANKTAVERQLEQTQQRFEVGLTAITDVHEAEAERDQALADEINAENTLDNNYESLRELTGIDHRQLDVLNTDRFSPQKTAFNSDKWLELALDKNLELHSARIGKDIAKEQIDLAKTGHEPTLDLGAGLNAGYNDYKDEIRNPESNSNQGNIGLNFKLPIYTGGATTSQVKQAQFNYVAASEQLERSFRSVQSTVRSSYNNVNASIGSVRAYSQSVISADSALKATEAGYEVGTRTIVDVLDSTRKLYQAKQKLSEARYNYILSILSLKQAAGILEQKDLEEVNQGLMPAAQVTKKS

SEQ ID NO: 335 - protein, 441 aa, Aeromonas hydrophila
MKRTLLSAMVLLGVSAGTHAENLLDIYQQAQIKDTQLLESKAKRDQAFEKINESRAALLPQINLGAGLNYLQNKGDTQTNSNATGSLSLDQSIYRRSNWVNLSLTEKSATQSDVAYNLEIQNLMLRTAQAYFNVLKAMDTLEFVRANKAAVERQLEQTQQRFEVGLTAITDVHEAEAERDQALADEINAENTLDNSYESLRELTGIDHRQLDVLNTDRFSPQKTPFNSDKWLELALDKNLQLHSARIGKDIAKEQIDLAKTGHEPTLDLGAGLSSTYSDYKDEIRNPESNANQANIGLNFKLPLYTGGATTSQVKQSQFNYVAASEQLERSFRNVQSTVRSSYNNVNASIGSVRAYGQSVISADSALKATEAGYEVGTRTIVDVLDSTRKLYQAKQKLSEARYNYILSILSLKQAAGTLEQKDLEDVNQGLMPASQAKSKS

SEQ ID NO: 336 - protein, 441 aa, Aeromonas salmonicida
MKRTLLSAMVLLGVSAGAHAENLLDIYQQAQIKDTQLQESKAKRDQAFEKINESRAALLPQINLGAGLNYLQNKGDTQTNSNATGSLSLDQSIYRRSNWVNLDLTEKSATQSDVAYNLEIQNLMLRTAQAYFNVLKAMDTLEFVRANKAAVERQLEQTQQRFEVGLTAITDVHEAEAERDQALADEINAENTLDNSYESLRELTGIDHRQLDVLNTERFSPQKTPFNSDKWLELALDKNLQLHSARIGKDIAKEQIDLAKTGHEPTLDLGAGLSSTYSDYKDEIRNPESNSNQGNIGLNFKLPLYTGGATTSQVKQSQFNYVAASEQLERSFRSVQSTVRSSYNNVNASIGSVRAYGQSVISADSALKATEAGYEVGTRTIVDVLDSTRKLYQAKQKLSEARYNYILSILSLKQAAGTLEQKDLEEVNQGLIPAAQVKNKS

SEQ ID NO: 337 - protein, 439 aa, Aeromonas veronii
MKRTLLSVMVLLGVSAGAHAENLLDIYQQAQIKDPQLLQSKAVRDQAFEKINESRAPLLPQIDLGAGLNYLQNKNDTQSNTNAKGEVTLSQSLYRRSNWVNLDISEKQATQSDVAYNLEIQNLMVRTAKAYFDVLKAMDTLEFVRANKVAVERQLEQTQQRFEVGLTAITDVHEAEASRDQALADEINAENSLDNSYETLRELTGIDHRNLDVLNTDRFSPQKTAFGSDKWLELALDKNLQLHSARISKDIAKEQIDLAKTGHEPTLDLGAGLQTSNTDYKLDSIPGGNDNQANIGLTFKLPIYSGGATSSKVKQAQYNYVAASELLEKNFRSVQSTVRSSYNNVNASIGSVRAYSQSVVSADSALKATEAGYEVGTRTIVDVLDSTRKLYEAKQKLSEARYNYILNILSLKQAAGILEAQDLVEVNQGLMPATAKAKG

SEQ ID NO: 338 - protein, 461 aa, Alcanivorax pacificus
MRTKRITYAAAALLLAATGVAQAATLADVLESAWQADSQWSGALRTWEAEQENVVQGRAGLLPSVNASYSWLDNDVEVTSSPGGETDFETQTLTLSLVQPLFRPGAWYAYKQADAATSVAAANFQQARQDFLQRVATQYFGVLQSWENLVSIRAEERAIGRQLEQTRERFDVGLVAITDVHEAEAAFDLTKVERILAEADFDIARDRLEALTGRTWESLAALQEELPLTGPEPANPQQWVELARQQNPTILAALYQSESSRHFASQQAWAHGPTVDLVAQHQRYKNDASGPTSTLNLPDTRTNAYGIEVNLPLFQGGGVNSRRKQAALQHEASQDLYQQTWRDVGQQTLGTYRVVSANALRIRARAQAIRSADSALEATQAGYEVGTRNIVDVLNAQRTLFAARRDYANARYEYILDSLGLKALAGVLTEDDLVQVNNWLAPADLVELDLVSAGTDDALDE

SEQ ID NO: 339 - protein, 462 aa, Alteromonas macleodii
MKRTLLSLVIGVSMTTGALADDLMQVYQQALANDPLVNQAKAQRDAAFEGISLSRASLLPQISGSVGYTHAESERFQQLGPDTSFTSDTTVDTTDYGISLTMSLYDHANWLNLNRAEKIAEQSDAQLAASMQDVIVRTVTAYFDVLRARDNVEFVGAEKRAIERQLEQTRQRFEVGLTAITDVHEAQANYDSTVAQEIQAKNQLEFALEALRVITGKYHDRLFGLNTENFSATMPVPETVDSWLETAQDQNLALLVDRLAMDVAKEDIAIARSGHLPTLALSGRYGRSKDDVEGEFITDDGQTAPVAYDTPYLDSKSIGVTLSVPIYQGGSVSAQTSQAKYNYVAASQAAEQTYRQTVQSVRSSFNNVKASISTIRALEQSVVSADSALKATEAGFDVGTRTIVDVLDSTRNLFDARRNLAGARYDFIQSVVTLKQAAGTLTGEDVAMINRGLKPAETKTNG

SEQ ID NO: 340 - protein, 457 aa, Alteromonas pelagimontana
MKRTLLSLIIGLGITSSALADDLLQVYQQALSSDPVVNRAQAERDAAYSGIPISRANLLPQIAGTVGYTFASRESTQFAGSDEQNLSLITLDSETESIDYGIDMSMSLYDHANWVGLNRAEKVAEQSDAQLAAAMQNLIVRTVTAYFDVLRAKDNLEFVRAEKRAIERQLEQTKQRFEVGLTAITDVHEAQANYDTTVAQEIQAENQVEVALEGLRVISGKYHDKLFTLDTDSFSASLPTPAKVEDWLSIAEDSNLSLLVNRLALDIAKEDIAAAQAGHYPSLGLSASAGRTKNDISSDNINYTTPYLNNYSVGVNLSVPIFQGLRVSSQTEQARYQYIAVSQDVELTYRQTVQSIRSSFNDLKAAISTIRALEQAVVSAKSALQATEAGFDVGTRTIVDVLDSTRNLFDARRNLASARYDFIQAVVDLKQAAGSLTSEDIVTINRGLEPQESKQPQ

SEQ ID NO: 341 - protein, 463 aa, Beggiatoa leptomitoformis
MMYKRLSYLLLTSCLVVPAIQAETLLDLYALAKENDAQLKVSGSDRLIALEAKPQAQASLLPQVVLGADATESWNTENWMSGSDTERTALGYSVSLSYPLYRRDRQILLEQADIGVKIAEESFASQQQALIQRLSSAYFTILNAEDNLRFTRSAREAFRRQLDQTQQRFDVGLIAITDVKEAQSGFDQAVSDEITAQNTVDQSYESLREITGSYHKLLATLKADTPLLPPDPQDIDAWTKTALEQNPQVIVQLATIENARQEVERQRAGNLPTVDLTAQHAYNDTVRGDESNGMRTNNSIGVSLSYSLYEGGAIRSKIREAQQRYVQELDRLEQVRRSIQNQTHTNYLNVLSGISRVKASRQAVESALTALEAIETGFDVGTRTSVDVLNARRDLLDAQRNYSQSRYQYVLSTVTLKQSAGLLRLEDLVAINNWLTTPSQVAEPATTEKKTEKETTKKSSAKK

SEQ ID NO: 342 - protein, 458 aa, Chania multitudinisentens
MKKLLPLLIGLSLGGFSAMSQAENLLQVYKQAKESNPDLRKTAADRDAAFEKISEARSPLLPQLGLSAGYNHTNGFRDANGVNSNTTNGSLSLTQTIFDMSAWRALTQQEKAAGISDVTFQTASQKLILDTATAYFNVLLAIDSLSYVQANKQAVYRTLDQTTQRFNVGLVAITDVQNARSNYDSVLASEVTARNNLDNSVEVLRQLTGTFYPELASLNTDKFSTQRPDAVNNLLKEAEKRNLSLLSARLNQDLAREQIKSAETGYMPTISASASTGLTNTRYNGSNSDSRTNSDNGQNQVGVTLSVPIYNGGVTNSRVQQAQYNFVGASEQLESTHRSVVQTVRSSFNNVSASISSISAYAQAVVSAQSSLDATEAGYEVGTRTIVDVLSATTTLYNAKQQLASARYSYLTNQLTIKSALGTLNENDLMLLNGVLGKTVPTSPDMVESATPQPARSN

SEQ ID NO: 343 - protein, 494 aa, Citrobacter sp.
MKKLLPILIGLSLSGFSTLSQAENLMQVYQQARLSNPELRKSAADRDAAFEKINEARSPLLPQLGLGADYTYSNGYRDANGVNSNATSASLQLTQSIFDMSKWRALTLQEKSAGIQDVTYQTDQQTLILNTATAYFNVLNAIDVLSYTQAQKDAVYRQLDQTTQRFNVGLVAITDVQNARSQYDTVLANEVTARNNLDNAVEQLRQVTGNYYPELASLNVDGFKTNKPQAVNALLKEAENRNLSLLQARLSQDLAREQIRLAQDGHLPTLDLTASTGVSDTSYSGSKTHGGATGNQYDDSNMGQNKIGLSFSLPLYQGGMVNSQVKQAQYNFVGASEQLESAHRSVVQTVRSSFNNINASISSINAYKQAVVSAQSSLDAMEAGYSVGTRTIVDVLDATTTLYNAKQQLANARYTYLINQLNVKSALGTLNEQDLVALNNTLGKPVSTTPDTIAPENAQQDAAADGYTSNSATPAAQPTAARSTSSNGNNPFRN

SEQ ID NO: 344 - protein, 493 aa, Citrobacter sp.
MKKLLPILIGLSLSGFSTLSQAENLMQVYQQARLSNPELRKSAADRDAAFEKINEARSPLLPQLGLGADYTYSNGYRDANGVNSNATSASLQLTQTLFDMSKWRTLTLQEKSAGIQDVTYQTDQQTLILNTATAYFKVLNAIDVLSYTQAQKDAVYRQLDQTTQRFNVGLVAITDVQNARSQYDTVLANEVTARNNLDNAVEELRQVTGNYYPELASLNVDGFKTNKPQAVNALLKEAENRNLTLLQARLSQDLAREQIRQAQDGHLPTLDLTASTGVSDTSYSGSKTRGATGSQYDDSNMGQNKIGLSFSLPLYQGGMVNSQVKQAQYNFVGASEQLESAHRSVVQTVRSSFNNINASISSINAYKQAVVSAQSSLDAMEAGYSVGTRTIVDVLDATTTLYNAKQQLANARYTYLINQLNVKSALGTLNEQDLVALNNTLGKPIPTSPDSVAPQNPQQDAAVNDFNSTGNMPAAQPTAARSTSSNGNNPFRN

SEQ ID NO: 345 - protein, 493 aa, Citrobacter sp.; identical to SEQ ID NO: 344

SEQ ID NO: 346 - protein, 493 aa, Citrobacter sp.
MQMKKLLPILIGLSLSGFSTLSQAENLMQVYQQARLSNPELRKSAADRDAAFEKINEARSPLLPQLGLGADYTYSNGFRDANGVNSNATSASLSLTQTLFDMSKWRALTLQEKSAGIQDVTYQTDQQTLILNTATAYFNVLNAIDVLSYTQAQKDAVYRQLDQTTQRFNVGLVAITDVQNARSQYDTVLANEVTARNNLDNAVEQLRQVTGNYYPELASLNVDGFKTNKPQAVNSLLKEAENRNLTLLQARLSQDLAREQIRLAQDGHLPTLGLTASTGVSDTSYSGSKTNTSQYDDSNQGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYNAKQQLANARYTYLINQLNIKSALGTLNEQDLVALNNTLGKPISTAPDAVAPENTQQDAAADGYTANSAAPAVKPATARTTSSSNGNNPFRN

SEQ ID NO: 347 - protein, 492 aa, Citrobacter sp.
MQMKKLLPILIGLSLSGFTTLSQAENLMQVYQQARLSNPELRKSAADRDAAFEKINEARSPLLPQLGLGADYTYSNGYRDANGVNSNATSASLQLTQTLFDMSKWRALTLQEKAAGIQDVTYQTDQQTLILNTATAYFNVLNAIDVLSYTQAQKDAVYRQLDQTTQRFNVGLVAITDVQNARSQYDTVLANEVTARNNLDNAVEQLRQVTGNYYPELASLNVDGFKTNKPQAVNALLKEAENRNLSLLQARLSQDLAREQIRLAQDGHLPTLGLTASTGVSDTSYNGSKTNTSQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYNAKQQLANARYTYLINQLNVKSALGTLNEQDLVALNNTLGKPIPTSPDNVAPQNPQQDAAVNDFNSNGNMPAAQPTAARSTSSNGNNPFRN

SEQ ID NO: 348 - protein, 493 aa, Citrobacter amalonaticus
MKKLLPILIGLSLSGFSTLSQAENLMQVYQQARLSNPELRKSAADRDAAFEKINEARSPLLPQLGLGADYTYSNGYRDANGVNSNATSASLQLTQTLFDMSKWRALTLQEKSAGIQDVTFQTDQQTLILNTASAYFKVLNAIDVLSYTQAQKEAIYRQLDQTTQRFNVGLVAITDVQNARSQYDTVLANEVTARNDLDNAVEELRQVTGNYYPELASLNVDSFKTDKPQAVNALLKEAENRNLTLLQARLSQDLAREQIRQAQDGHLPTLDLTASTGVSDTSYSGSKTRGATGSQYDDSNMGQNKIGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQQLANARYNYLINQLNIKSALGTLNEQDLVALNNTLGKPISTSPEHVAPETPQMDANADGYATNAAAPAAQPASARSSSSNGNNPFRN

SEQ ID NO: 349 - protein, 616 aa, Coxiella burnetii
MKKKFLVFVLILFSLVAAPLLYAEDLVQVYCQALASDPTFQKAHADWLSARQNLPIAMSGTGTPGSGLFPYVDITAGLDRTFQRIEAGSSSVSGYFNQHNYQVTVTQPIFNYATWKAISSASFSVKAATATYIEAAQDLIFRTAKAYFDVLDAYDQLQFTLAQKESFYHQLVTAQEKFKVGLIAITGVYDAQASYDQAIAQEIQDRNNLDNQLENLRAITGQEYRSLTSLKKSIPLVIPHPRNIDAWTAVAERQSFAIQSALYTMLAQRETVKETAAQRYPTLTGTFSYGAQQRGFPPSLSGPPTTGEFLDTTTTTATAGLNLNFPVFQGGFVTHSTRQEEYNYLSASDQLNFTHRDVVRQTRQAYLGVDSGISKIRADRQAIISAQNKLEATQAGYVVGTRTMVDVLDAVTSLYQAQQQWATDRYSYIINIITLKQQAGTLCPHDLAQINTWLGKAVRFDVEKPVNAKVFTPSTTLPHTVKHVHSPKGVQRATRSHRPLIKASRHHTSPVHSASHIETVHAHYTIQLFASRTLAEATAFKNKHRLHDLRIIHQNGWYKVLSGHYSTRQAATIALHRLPSSLQKLKPWVVRVPKVQSTSIKPLSLQKKATHLPPPR

SEQ ID NO: 350 - protein, 495 aa, Cronobacter malonaticus
MKKLLPILIGLSLTGFSAMSQAENLLQVYQQARLSNPDLRSSAADRDAAFEKINEARSPLLPQLGLGADYTYTNGFRDNDGVDNTVKSASLQLTQTIFDMSKWRALTLQEKTAGIQDVTYQTDQQTLMLNTATAYFQVLSAIDALSYTEAQKQAIYRQLDQTTQRFNVGLVAITDVQNARAQYDNVLANEVTARNNLDNALEQLRQVTGNYYPQLASLNVDNFKTTKPAAINALLKEAEQRNLTLLQARLSQDLAREQIRYAETGHMPTLGLTASSSVSDTDYSGSKTSGAAASRYADSKIGQNSIGLSFNLPLYSGGSVTSQVKQAQYSFVGASEKLESAHRNVVQTVRSSYNNVNASISSIKAYEQAVVSAQSSLDAMEAGYSVGTRTIVDVLDATTTLYNAKQQLSSARYNYLINQLNIKSALGTLNEQDLVALNNSLGKPVSTAPESVAPENPEQDAAVNNMANGGGSAPAMQPAAATRSGNSNSGNPFRQ

SEQ ID NO: 351 - protein, 470 aa, Dickeya sp.
MKKLLPLLISLGLVGFSAASQAEDLLQVYQQAKTTNPDLRSAAANRDAAFEKINEARSSLLPQLGLGADYTYNSGYRDRKDVNNNVTSASLQLTQSIFNMSLWRSLTLQEKQAGIQDVSYQTSQQTLLLNTATAYFNVLRTIDALSYVSAQKKAIYRELDQTTQRFNVGLVAITDVQNARAQYDSVLANEVTARNNLDNAMENLRQVTGQFYPQLAALNIEHFSQQKPDAVNGLLKEAENRNLSLLSARLSQDLAREQIRSAESGHLPTLNLTASTGVTNTNYSGSRVAASNLTDTYVGQNQVGLSLSVPIYSGGGTSSKVKQAQYSFVASSENLESAHRSVIQTLRSAHNNISASISSVNAYKQAVVSAQSSLDAMEAGYQVGTRTIVDVLDATSTLYNAKQQLSGARYDYLINQLNLKQALGTLNEDDLRTLNAMLGKPVPTSAAIGDDTPVPAATPVSVTTTTKARP

SEQ ID NO: 352 - protein, 470 aa, Dickeya solani
MKKLLPLLISLGLGGFSAVSQAEDLLQVYQQAKTTNPDLRSAAANRDAAFEKINEARSSLLPQLGLGADYTYNSGYRDRRDINSNATSASLQLTQSIFNMSLWRALTLQEKQAGIQDVSYQTSQQTLLLNTATAYFSVLRAIDSLSYVSAQKQSIYRELDQTTQRFNVGLVAITDVQNARAQYDSVLASEVTARNNLDNAMETLRQVTGQFYPQLAGLNIDRFSPQKPDAVNGLLKEAENRNLSLLSARLSQDLAREQIRSAESGHLPTLNLTASTGVTNTDYSGSRAVGDSFNNTYVGQNQVGLSFSLPIYSGGGTSSRVKQAQHSFVASSESMESAHRSVIQTLRSAHNNISASISSVNAYKQAVISAQSSLDAMEAGYQVGTRTIVDVLDATTTLYNAKQQLSGARYDYLINQLNLKQALGTLNEDDLRTLNAMLGKPVSTSAAIGDDTPAPATTPAAAKTAAKAKP

SEQ ID NO: 353 - protein, 475 aa, Edwardsiella sp.
MKKLLPLLIGLSLSGFSALSQAENLMQVYQQAKESNPDLRQAAANRDAAFEKINESRSPLLPQLGLGADYTYTNGFRDSSGLNSNNYGATLALTQTIFDMSKWRQLSLTEKQAGIQDVSYQSSEQTLMLNTATAYFNVLRAIDQLTYTEANKQAIYNQLDQTTQRFNVGLVAITDVQNARAQYDQVLAQEVSARNNLDNSLEALRQITGQYYPQLSALNIDRFSTKRPDDVKALLQEAEKRNLQLLSARLSQDLARQQIKYAETGYMPTLNLTASTGVSNTDYNSLSNAQKETLRGGNGSSYQGQNTVGLSFNLPLYSGGATNSQVKQAQYNFVGASEALDSAHRSVVQNVRSSFNNISAAISGVSAYKQAVVSAQSSLDATQAGYQVGTRTIVDVLNATTALYNAKQNLANARYDYLISQLNIKYALGTLNQNDLLALNGDLSKPVPTAADSIAPDDAAQAAKANGPIPGGGVR

SEQ ID NO: 354 - protein, 475 aa, Edwardsiella sp.
MKKLLPLLIGLSLSGFSALSQAENLMQVYQQAKESNPDLRQAAANRDAAFEKINESRSPLLPQLGLGADYTYTNGFRDSSGLNSNNYGATLALTQTIFDMSKWRQLSLTEKQAGIQDVSYQSSEQTLMLNTATAYFNVLRAIDQLTYTEANKQAIYNQLDQTTQRFNVGLVAITDVQNARAQYDQVLAQEVSARNNLDNSLEALRQITGQYYPQLSTLNIDRFSTKRPDDVKALLQEAEKRNLQLLSARLSQDLARQQIKYAETGYMPTLNLTASTGVSNTDYNSLSNAQKETLRGGNGSSYQGQNTVGLSFNLPLYSGGATNSQVKQAQYNFVGASEALDSAHRSVVQNVRSSFNNISAAISGVSAYKQAVVSAQSSLDATQAGYQVGTRTIVDVLNATTALYNAKQNLANARYDYLISQLNIKYALGTLNQNDLLALNGDLSKPVPTAADSIAPDDAAQAAKANGPIPGGSVR

SEQ ID NO: 355 - protein, 475 aa, Edwardsiella ictaluri
MKKLLPLLIGLSLSGFSALSQAENLMQVYQQAKESNPDLRQAAANRDAAFEKINESRSPLLPQLGLGADYTYTNGFRDSSGLNSNNYGATLALTQTIFDMSKWRQLSLTEKQAGIQDVSYQSSEQTLMLNTATAYFNVLRAIDQLTYTEANKQAIYNQLDQTTQRFKVGLVAITDVQNARAQYDQVLAQEVSARNNLDNSLEALRQITGQYYPQLSALNIDRFSTKRPDDVKALLQEAEKRNLQLLSARLSQDLARQQIKYAETGYMPTLNLTASTGVSNTDYNSLSNAQKETLRGGNGSSYQGQNTVGLSFNLPLYSGGATNSQVKQAQYNFVGASEALDSAHRGVVQNVRSSFNNISAAISSVSAYKQAVVSAQSSLDATQAGYQVGTRTIVDVLNATTALYNAKQNLANARYDYLISQLNIKYALGTLNQNDLLMLNGDLSKTVPTAADSIAPDDAAQAAKANGPIPGGGVR

SEQ ID NO: 356 - protein, 492 aa, Enterobacter sp.
MKKLLPILIGLSLTGFSAMSQAENLLQVYQQARLGNPDLRKSAADRDAAFEKINEARSPLLPQLGLGADYTYTNGFRDNNGINSNATSASLQLTQTLFDMSKWRELSLQEKSAGIQDVTYQTDQQTLILNTATAYFNVLSAIDALSYTEAQKQAIYRQLDQTTQRFNVGLVAITDVQNARSQYDTVLANEVTARNNLDNALEALRQVTGNYYPELASLNVDSFKTDKPQAVNALLKEAENRNLTLLQARLSQDLAREQIRLAQDGHLPTLSLSASTGVSDTSYSGSKTNTAQYDDNNMGQNKVGLSFSLPLYQGGMVNSQVKQAQYNFVGASEQLESAHRNVVQTVRSSFNNVNASISSINAYKQAVVSAQSSLDAMEAGYSVGTRTIVDVLDATTTLYNAKQQLSSARYQYLINQLNIKQALGTLNEQDLQMLNSTLGKPVSTSPDSVAPENPQQDAAVDNFTANSSTPVAQPAAARSTSPASSGTNPFRN

SEQ ID NO: 357 - protein, 492 aa, Enterobacter sp.
MKKLLPILIGLSLTGFSAMSQAENLLQVYQQARLGNPDLRKSAADRDAAFEKINEARSPLLPQLGLGADYTYSNGFRDANGVNSNATSASLQLTQTLFDMSKWRALTLQEKSAGIQDVTYQTDQQTLILNTATAYFNVLSAIDALSYTEAQKQAIYRQLDQTTQRFNVGLVAITDVQNARSQYDTVLANEVTARNNLDNALESLRQVTGNYYPELASLNVDSFKTDKPQAVNALLKEAENRNLSLLQARLSQDLAREQIRQAQDGHLPTLSLSASTGVSDTTYSGSKTNTSQYDDSNMGQNKVGLSFSLPLYQGGQVNSQVKQAQYNFVGASEQLESAHRNVVQTVRSSFNNVNASISSINAYKQAVVSAQSSLDAMEAGYSVGTRTIVDVLDATTTLYNAKQQLSSARYQYLINQLNIKQALGTLNEQDLQMLNSALGKPVSTSPDSVAPENPEQVAAVDNFNANSNTPAAQPAAARTTTSASKGNNPFRN

SEQ ID NO: 358 - protein, 492 aa, Enterobacter cloacae
MKKLLPILIGLSLTGFSAMSQAENLLQVYQQARLGNPDLRKSAADRDAAFEKINEARSPLLPQLGLGADYTYTNGFRDNNGIDSNATSASLQLTQTLFDMSKWRELSLQEKSAGIQDVTYQTDQQTLILNTATAYFQVLSAIDALSYTEAQKQAIYRQLDQTTQRFNVGLVAITDVQNARSQYDTVLANEVTARNNLDNALEALRQVTGNYYPELASLNVDSFKTDKPQAVNALLKEAENRNLTLLQARLSQDLAREQIRLAQDGHLPTLSLSASTGVSDTSYSGSKTTSQAYDDSNVGQNKVGLSFSLPLYQGGMVNSQVKQAQYNFVGASEQLESAHRNVVQTVRSSFNNVNASISSINAYKQAVVSAQSSLDAMEAGYSVGTRTIVDVLDATTTLYNAKQQLSSARYQYLINQLNIKQALGTLNEQDLQMLNSTLGKPVSTSPDSVAPENPEQDAAVDNFNANGSAPAAQPAAARSTSPASSGTNPFRN

SEQ ID NO: 359 - protein, 492 aa, Enterobacter cloacae
MKKLLPILIGLSLTGFSAMSQAENLLQVYQQARLGNPDLRKSAADRDAAFEKINEARSPLLPQLGLGADYTYSNGFRDANGVNSNATSASLQLTQTLFDMSKWRALTLQEKSAGIQDVTYQTDQQTLILNTATAYFNVLSAIDALSYTEAQKQAIYRQLDQTTQRFNVGLVAITDVQNARSQYDTVLANEVTARNNLDNALESLRQVTGNYYPELASLNVDSFKTDKPQAVNALLKEAENRNLSLLQARLSQDLAREQIRQAQDGHLPTLSLSASTGVSDTTYSGSKTNTSQYDDSNLGQNKVGLSFSLPLYQGGQVNSQVKQAQYNFVGASEQLESAHRNVVQTVRSSFNNVNASISSINAYKQAVVSAQSSLDAMEAGYSVGTRTIVDVLDATTTLYNAKQLLSSARYQYLINQLNIKQALGTLNEQDLQMLNSALGKPVSTSPDSVAPENPQQVAAVDNFNANGSAPAAQPAAARTTAPASKGNNPFRN

SEQ ID NO: 360 - protein, 492 aa, Enterobacter cloacae
MKKLLPILIGLSLTGFSAMSQAENLLQVYQQARLGNPDLRKSAADRDAAFEKINEARSPLLPQLGLGADYTYSNGFRDANGVNSNATSASLQLTQTLFDMSKWRALTLQEKSAGIQDVTYQTDQQTLILNTATAYFNVLSAIDALSYTEAQKQAIYRQLDQTTQRFNVGLVAITDVQNARSQYDTVLANEVTARNNLDNALESLRQVTGNYYPELASLNVDSFKTDKPQAVNALLKEAENRNLTLLQARLSQDLAREQIRQAQDGHLPTLSLSASTGVSDTTYSGSKTNSAQYDDSNMGQNKVGLSFSLPLYQGGQVNSQVKQAQYNFVGASEQLESAHRNVVQTVRSSFNNVNASISSINAYKQAVVSAQSSLDAMEAGYSVGTRTIVDVLDATTTLYNAKQQLSSARYQYLINQLNIKQALGTLNEQDLQMLNSALGKPVSTSPDSVAPENPEQVAAVDNFNANSTTPAAQPAAARTTAPASKGNNPFRN

SEQ ID NO: 361 - protein, 492 aa, Enterobacter sp.
MKKLLPILIGLSLTGFSAMSQAENLLQVYQQARLGNPDLRKSAADRDAAFEKINEARSPLLPQLGLGADYTYTNGFRDNNGIDSNATSASLQLTQTLFDMSKWRELSLQEKSAGIQDVTYQTDQQTLILNTATAYFQVLSAIDALSYTEAQKQAVYRQLDQTTQRFNVGLVAITDVQNARSQYDTVLANEVTARNNLDNALEALRQVTGNYYPELASLNVDSFKTDKPQAVNALLKEAENRNLTLLQARLSQDLAREQIRLAQDGHLPTLSLSASTGVSDTSYSGSKTTSQAYDDSNVGQNKVGLSFSLPLYQGGMVNSQVKQAQYNFVGASEQLESAHRNVVQTVRSSYNNVNASISSINAYKQAVVSAQSSLDANEAGYSVGTRTIVDVLDATTTLYNAKQQLSSARYQYLINQLNIKQALGTLNEQDLQMLNSTLGKPVPTSPDSVAPENPQQDAAVDNFTPNSSAPLAQPAAARSTATASSGTNPFRH

SEQ ID NO: 362 - protein, 491 aa, Enterobacter sp.
MKKLLPILIGLSLAGFSAMSQAENLLQVYKQARLSNPELRRSAADRDAAFEKINEARSPLLPQLGLGADYTYSNGFRDSNGVNSNATSASLQLTQTLFDMSKWRALTLQEKAAGIQDVTFQTDQQTLILNTATAYFNVLSAIDSLSYTEAQKQAIYRQLDQTTQRFNVGLVAITDVQNARSQYDTVLANEVTARNNLDNAVEQLRQVTGNYYPELASLNVDNFKTDKPQPVNALLKEAENRNLSLLQARLNQDLAREQIRQAQDGHLPTLDLTASTGVSDTSYSGSKTNNAQFDDSNVGQNKVGLNFSLPIFQGGMVNSQVKQAQYNFVGASEQLETAHRSVVQNVRSSFNNVNASISSIKAYEQAVVSAQSSLDASEAGYSVGTRTIVDVLDATTALYNAKQQLSSARYNYLINQLNIKSALGTLNEQDLLALNSTLGKPTSTTPEAVAPENPQQDARVQNIAADAQSATTADAPVATNSSRSGANPFSR

SEQ ID NO: 363 - protein, 492 aa, Enterobacter sp.
MKKLLPILIGLSLTGFSAMSQAENLLQVYQQARLGNPDLRKSAADRDAAFEKINEARSPLLPQLGLGADYTYSNGFRDANGVNSNATSASLQLTQTLFDMSKWRALTLQEKTAGIQDVTYQTDQQTLILNTATAYFNVLSAIDALSYTEAQKQAIYRQLDQTTQRFNVGLVAITDVQNARSQYDTVLANEVTARNNLDNALESLRQVTGNYYPELASLNVDSFKTDKPQAVNALLKEAENRNLTLLQARLSQDLAREQIRQAQDGHLPTLSLSASTGVSDTTYSGSKTNSAQYDDSNMGQNKVGLSFSLPLYQGGQVNSQVKQAQYNFVGASEQLESAHRNVVQTVRSSFNNVNASISSINAYKQAVVSAQSSLDAMEAGYSVGTRTIVDVLDATTTLYNAKQQLSSARYQYLINQLNIKQALGTLNEQDLQMLNSALGKPVSTSPDSVAPENPDQVAAVDNFNANGSTPAAQPAAERTTAPASKSNNPFRN

SEQ ID NO: 364 - protein, 492 aa, Enterobacterales sp.
MKKLLPILIGLSLTGFSAMSQAENLLQVYQQARLGNPDLRKSAADRDAAFEKINEARSPLLPQLGLGADYTYSNGFRDANGVNSNATSASLQLTQTLFDMSKWRALTLQEKSAGIQDVTYQTDQQTLILNTATAYFNVLSAIDALSYTEAQKQAIYRQLDQTTQRFNVGLVAITDVQNARSQYDTVLANEVTARNNLDNALESLRQVTGNYYPELASLNVDSFKTDKPQAVNALLKEAENRNLSLLQARLSQDLAREQIRQAQDGHLPTLSLSASTGVSDTSYSGSKTNTSQYDDSNMGQNKVGLSFSLPLYQGGQVNSQVKQAQYNFVGASEQLESAHRNVVQTVRSSFNNVNASISSINAYKQAVVSAQSSLDAMEAGYSVGTRTIVDVLDATTTLYNAKQQLSSARYQYLINQLNIKQALGTLNEQDLQMLNSALGKPVSTSPDSVAPENPEQVAAVDNFNANSSTPAAQPAAARTTTSASKGNNPFRN

SEQ ID NO: 365 - protein, 493 aa, Enterobacteriaceae sp.
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNTLLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 366 - protein, 493 aa, Enterobacteriaceae sp.
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTHQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 367 - protein, 494 aa, Enterobacteriaceae sp.
MKKLLPILIGLSLSGFSTLSQAENLMQVYQQARLSNPELRKSAADRDAAFEKINEARSPLLPQLGLGADYTYSNGYRDANGVNSNATSASLQLTQTLFDMSKWRALTLQEKSAGIQDVTYQTDQQTLILNTATAYFNVLNAIDVLSYTQAQKDAVYRQLDQTTQRFNVGLVAITDVQNARSQYDTVLANEVTARNNLDNAVEQLRQVTGNYYPELASLNVDGFKTNKPQAVNALLKEAENRNLTLLQARLSQDLAREQIRQAQDGHLPTLDLTASTGVSDTSYSGSKTHGGTTGNQYDDSNMGQNKIGLSFSLPLYQGGMVNSQVKQAQYNFVGASEQLESAHRSVVQTVRSSFNNINASISSINAYKQAVVSAQSSLDAMEAGYSVGTRTIVDVLDATTTLYNAKQQLANARYTYLINQLNVKSALGTLNEQDLVALNNTLGKPVSTTPDTVAPQNPQQDAAVNDFNGTGNLPAAQPTAARSTSSNGNNPFRN

SEQ ID NO: 368 - protein, 493 aa, Escherichia sp.
MKKLLPILIGLSLSGFSTLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQALILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNTLLKEAEKRNLTLLQARLSQDLAREQIRLAQDGHLPTLNLTASTGISDTSYSGSKTRGANSAQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYTPDSPAPVVQPASVRTNTSTGKNPFRN

SEQ ID NO: 369 - protein, 493 aa, Escherichia albertii
MKKLLPILIGLSLSGFSTLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQALILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNTLLKEAEKRNLTLLQARLSQDLAREQIRQAQDGHLPTLNLTASTGISDTSYSGSKTRGANSAQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYTPDSPAPVVQPASVRTNTSTGKNPFRN

SEQ ID NO: 370 - protein, 493 aa, Escherichia albertii
MKKLLPILIGLSLSGFSTLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQALILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNTLLKEAEKRNLTLLQARLSQDLAREQIRQAQDGHLPTLNLTASTGISDTSYSGSKTRGANSAQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYTPDSPVPVVQPASVRTNTSTGKNPFRN

SEQ ID NO: 371 - protein, 493 aa, Escherichia albertii
MKKLLPILIGLSLSGFSTLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQALILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNTLLKEAETRNLTLLQARLSQDLAREQIRQAQDGHLPTLNLTASTGISDTSYSGSKTRGANSAQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYTPDSPVPVVQPASVRTNTSTGKNPFRN

SEQ ID NO: 372 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTFARTTTSNGHNPFRN

SEQ ID NO: 373 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPVPVVQQTSARTTTSNGHNPFRN

SEQ ID NO: 374 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNTLLKEAEKRNLSLLQARLSQDLAREQIRQAQDGHLPTLDLTASTGISDTSYSGSKTRGATAGQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 375 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYTELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 376 - protein, 493 aa, Escherichia coli
MKKLLPILIGLSLYGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 377 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGSLNEQDLLALNNALSKPVSTNPENVAPQTPEQNAIADGYAPDSPAPVVQQTSARTTTSNGHNPFRN

SEQ ID NO: 378 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNAASASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 379 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASSGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 380 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 381 - protein, 491 aa, Escherichia fergusonii
MKKLLPILIGLSLSGFSALSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNIDNFKTDKPQPVNALLKEAEKRNLSLLQARLSQDLAREQIRQAQDGHLPTLDLTASTGISDTSYSGSKTRGANSAQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQQLANARYNYLINQLNIKSALGTLNEQDLIALNNALSKPVSTNPENVAPQTPEQNAIADGYAPDSPAPVVQQTSVRTSNGNNPFRN

SEQ ID NO: 382 - protein, 438 aa, Grimontia hollisae
MKKLLPFFIGVALGTAAPLASADDLIQVYEQAKQSDPQLLQAAATKDAAFAAVDTNRGTLLPQINLQAGYNLSRNSDNIVSDTNTLTAGVTLKQELFEQNSWINLDIAEITARQADAVYAAEQQGLILRVSQAYFNVLRAIDDLIFVRAEKAAVGRQLEQTKQRFEVGLSAITDVHDAQAQYDAVLAQEILSENEVSNSYEALREITGTRHSDLYILNTKTFSATRPQQTVEELVKRAEEGNLNLLAQRISRDAAKERISLAESGHLPSLTLDVGYGYTDQENKTDSSRDINNNELAGGINFNMPLYSGGSISAGVKEAQFRYVAASEQLEGTYRTTVKDVRAFYNNINASIGALRAYEQTVISQRSALEATEAGFEVGTRTIVDVLDATRRLYDANRNLSNARYDYILNVLQLRQAIGTLSEQDLVDINNGLLPPKK

SEQ ID NO: 383 - protein, 498 aa, Hafnia alvei
MKKLLPLLIGLSLGGFTTLSHAENLLQVYQQAKESNPDLRKAAADRDVAFEKINESRSPLLPQLGLGADYTYTNGMRDNDGLNSNNYGASLQLTQTIFDMSKWRQLSLTEKQAGIQDVSYQSSEQTLMLNSATAYFNVLRAIDALSYIEANKEAIYRQLDQTTQRFNVGLVAITDVQNARAQYDLVLANEVTARNDLDNSLEALRQITGQYYGQLDSLNVDRFTTTKPDNVNSLLQEAEKRNLSLLSARLSQDLAREQIKYAQTGYMPTLDLTASSGVSNTDYNSLSNSQKAQNQNGASSYQGQNTIGLQFNLPLYSGGATNSQVQQAQYNFVGASESLESAHRSVVQNVRSSFNNISASISGVEAYRQAVISAQSSLEATEAGYQVGTRTIVDVLNATTTLYSAKESLSNARYDYLISQLNIKYALGTLNQNDLMALNGGLTKPVSTTPDIIAPQGKTQAAQVNSDYNTTASAPVAQPASASTTTSSSSKGKNPFRH

SEQ ID NO: 384 - protein, 493 aa, Hafnia paralvei
MKKLLPLLIGLSLGGFTTLSHAENLMQVYQQAKESNPDLRKAAADRDVAFEKINESRSPLLPQLGLGADYTYTNGMRDNDGLNSNNYGASLQLTQTIFDMSKWRQLSLTEKQAGIQDVSYQSSEQTLMLNSATAYFNVLRAIDALSYIEANKEAIYRQLDQTTQRFNVGLVAITDVQNARAQYDLVLANEVTARNNLDNSLEALRQITGQYYGQLDSLNVERFSTSKPENVNSLLQEAEKRNLSLLSARLSQDLAREQIKYAQTGYMPTLDLTASSGVSNTDYNSLSNSQKAQNSNGASSYQGQNTIGLQFNLPLYSGGATNSQVQQAQYNFVGASESLESAHRSVIQNVRSSFNNISASISGVEAYRQAVVSAQSSLEATEAGYQVGTRTIVDVLNATTTLYSAKESLSNARYDYLISQLNIKYALGTLNQNDLMALNGGLTKPVSTTPDIIAPQGKTQADQVNSDYSAAPVAQATPVSSTSSSKGKNPFRN

SEQ ID NO: 385 - protein, 487 aa, Halomonas sp.
MRLAHWAVNAPLRPLLLAVFTASFVLPAQAADLITITRDALDNNAELASARSEYLGVEAGRDVARGGLLPQINASGTVAHNEQYESQGSSRAGTGAGTGNVSAGDDRYNTVALTLEATQALYDEVTRRQVTQAERQIDQQVYLLAATEQQLLIDVASAYFDILRAYEVLEARFAQERAIGRQLEQAGEQFEVGLIAITEVEEARATFDQSRADRIAAESNLQVAFEALEQLTGQRYASIEALGDSMPIALPEPSNRDYWVEQAIERNPQVLAQQAGIEVSRSGVEIARAGRLPTLSAFGNYQYGDSDIDNTTGNDSSSQVGIRANLPVYTGGSTSASIRQGTYQLESSQYDFESQRRSSIQQVRSLYTQVSNDVETVEAREQAIVSNQSALEATRAGYEVGTRNIVDVLNAEQNLYNAIANYAEARYDYVVNLLSLRQQAGRLDVDAIEEVNAWLTGDEVNFTLPESGGNDRYESALNIGAPPQPGT

SEQ ID NO: 386 - protein, 481 aa, Halomonas sp.
MKPYRPVSSGRRVLLPCLLAVSLAGQAQAADLLTITRDAIDNNASLASARAQYGSVEAGRDVARGDLLPQVSASGQVDHNRLYESVSSSRGSGAGNGDDSYNSASLTLEATQALFDAANSAEVDQAASRIDQQTYQLAATEQQVLIDVASAYFDILRAHEILEARRAQERAIGRQLEQAQEQFDVGLIAITEVEEARASFDQSRADRIAAENDLQVSFETLERLTGQRYDSIDALQEAMPVTPPQPSGRDAWVELAMDNSPLVLASQAGVEVSEDAVDIARAQRLPVVEAFANYSYSDTDQDQMDGHNSQSQVGARVSLPIYTGGRTSAGIRQSTYDLESSQYDFEDQRRQTVQQVRSLYTQVTNDVATVEARAQAIVSNQSALDATRAGYEVGTRNIVDVLNAEQSLYDAIADHADARFSYVIDLLTLRQQSGTLNVQAIEAINDWLSAEQGVSLALPEVGEASRYDEAMNIGAPPRPES

SEQ ID NO: 387 - protein, 481 aa, Halomonas elongata
MPSRPVTPPSRVARRALLPMLVASLMAGQVQAADLLTITRDALDNNADLAAARSDTDSVEAGRDVERGDLLPQVNASGQVAHNEQFESQSSAFSAGSTPSDDRYNSASLTLEASQVLYDAVNSAELEQADRQIDQQTYQLAATEQQVLIDVASAYFDILRAHEILEARRAQERAIGRQLEQAREQFDVGLIAVTEVEEAQASYDQSRADRISAESDLQVAFEALERLTGKRYDSIESLEDAMPVTPPEPSKRDAWIDLAMENNPLVLAQQAGVEVSRSGVDVSKAQRLPVVNAFANYQYADSDNDTLEGEDSSAQVGVKLSVPIYTGGRTSAGIRQSTYALESSQYNFEAQRRDTIQQVRSLYTQVTNDVATVEARAQAIVSNQSALDATRAGYEVGTRNIVDVLNAEQNLYDAIANHADARYDYVINLLTLRQQSGTLDVDAIEEVNGWLDENQSVSFTLPEEGRYEQALDIGEPPRPDA

SEQ ID NO: 388 - protein, 487 aa, Halomonas sp.
MRLAHWAVNAPLRPLLLAVFTASFVLPAQAADLLTITRDALDNNAALASARSEYLGVEAGRDVARSGLLPQVNATGNVAHNQQYESKTSSRAGTGTGTGNVSTGDDRYNTVSLTLEATQALYNEVTRREVTQAERQIDQQVYLLAATEQQLLIDTASAYFDILRAHEILEARFAQERAIGRQLEQAGEQFEVGLIAITEVEEARATFDQSRADRIAAESNLQVAFEALEQLTGQRYASIEALGDSMPVGLPEPSSRDYWVEQALELNPQVLAQQAGIEVARSGVEIARAGNLPTLQAFGNYQYSDSDVDNTTGEDSSSQVGLSANLPIYTGGRTSASIRQGTYQLESSQYDFESQRRSSIQQVRSLYTQVSNDVETVEARQQAIVSNQSALEATRAGYEVGTRNIVDVLNAEQNLYNAIANYAEARYDYVVNLLSLRQQAGRLDVEAIEEVNAWLTGDEVNFILPESDGNDRYDSALDIGAPPQPGM

SEQ ID NO: 389 - protein, 484 aa, Halomonas sp.
MTPSRLDSPAARPNMSRRLLGAMVAALLAGQVQAADLLTITRDALDNNSDLASARATTSGTEENRNVARADLLPQLTASGEVAYNSVFEQQSQKGFSGTVGDDRYTSYGLNLEATQAIYDARNSREVGVAEKQIDQQTYVLAATEQQVLIDTATAYFDILRANDILDARRAQERAIGRQLEQVQEQFDVGLVAITDVEEARATFDQSRADRISAESDLQVAFEALERLTGKRYDSIDKLQDELPIALPTPSSRDAWIDTAIESSPDLLAQKSGVDVAREQVSVSKADRLPVVSGFVNYSYADDDQDYLEDYNSTGQVGVNASLPLYTGGRTSASIRQNTYNLESSQYDFESQRRETIQQVRALYTQVSNDVSTVAARQQTIVSSQSALDATRAGYEVGTRNIVDVLSAEQNLYNAIANYADARYSYVTNLLTLRRQAGVLDEAAIETINQYLQSSGQVTFTLPENNGGTYDAALDIGERPQLEQ

SEQ ID NO: 390 - protein, 490 aa, Klebsiella sp.
MKKLLPILIGLSLTGFSAMSQAENLLQVYQQARISNPDLRKSAADRDAAFEKINEARSPLLPQLGLGADYTYNNGYRDSNGINSNVTSGSLQLTQVLFDMSKWRALTLQEKTAGIQDVTYQTDQQTLILNTATAYFKVLAAIDTLSYTEAQKQAIYRQLDQTTQRFNVGLVAITDVQNARSQYDAVLANEVTARNDLDNAVEELRQVTGNYYPELASLNVNGFKTNKPQAVNALLKEAENRNLSLLQARLNQDLAREQIRQAQDGHLPTLSLSASSGISNTSYSGSKTHNNPQQYQDNDAGQNQIGLNFSLPLYQGGAVTSQVKQAQYNFVGASEQLESAHRSVVQTVRSSFNNVNASISSINAYKQAVVSAQSSLDAMEAGYSVGTRTIVDVLDATTTLYNAKQQLSNARYNYLINELNIKSALGTLNEQDLVALNNTLGKPISTSADSVAPENPQQDATADGYGNTTAAVKPASARTTQSSGSNPFRQ

SEQ ID NO: 391 - protein, 486 aa, Klebsiella sp.
MKKLLPILIGLSLTGFSAMSQAENLLQVYQQARISNPDLRKSAADRDAAFEKINEARSPLLPQLGLGADYTYTSGFRDYKDQNSNVTSGSLQLTQVLFDMSKWRALTLQEKAAGIQDVTYQTDQQTLILNTATAYFKVLAAIDTLSYTEAQKQAIYRQLDQTTQRFNVGLVAITDVQNARSQYDAVLANEVTARNDLDNAVEELRQVTGNYYPELASLNVDGFKTSKPQAVNALLKEAENRNLSLLQARLNQDLAREQIRQAQDGHLPTLDLNASSGVSNNRYSGSKSISQDADIGQNKIGLSFSLPLYQGGMVNSQVKQAQYNFVGASEQLESAHRSVVQTVRSSFNNVNASISSINAYKQAVVSAQSSLDAMEAGYSVGTRTIVDVLDATTTLYNAKQQLSNARYNYLINELNIKSALGTLNEQDLIALNNTLGKPISTSADSVAPENPQQDATADGYGNTTAAMKPASARTTTHSSGSNPFRQ

SEQ ID NO: 392 - protein, 482 aa, Klebsiella sp.
MKKLFPILIGLGLTGFSAMSQAENLLQVYQQARVSNPDLRKSAADRDAAFEKINEARSPLLPQLGLGADYTYTSGFRDAKDQNSDVTSGSLQLTQVLFDMSKWRALTLQEKNAGIQDVTYQTDQQTLILNTATAYFKVLAAIDTLSYTEAQKQAIYRQLDQTTQRFNVGLVAITDVQNARSQYDAVLANEVTARNDLDNAVEGLRQVTGNYYPELASLNVDGFKTTKPSAVNALLKEAENRNLSLLQARLSQDLAREQIRQAQDGHLPTLNLNASSSVSNNSYSGSKNTTQDRDIGQNQIGLNFSLPLYQGGMVNSQVKQAQYNFVGASEQLESAHRSVVQTVRSSFNNVNASISSINAYKQAVVSAQSSLDAMEAGYSVGTRTIVDVLDATTTLYNAKQQLSNARYNYLINELNIKSALGTLNEQDLVALNNTLGKAIPTSPDSVAPENPQQDASADGYSNTAAAKPASARSTSGSNPFRQ

SEQ ID NO: 393 - protein, 490 aa, Klebsiella sp.
MKKLLPILIGLSLTGFSAMSQAENLLQVYQQARISNPDLRKSAADRDAAFEKINEARSPLLPQLGLGADYTYNNGYRDSNGINSNVTSGSLQLTQVLFDMSKWRALTLQEKTAGIQDVTYQTDQQTLILNTATAYFKVLAAIDTLSYTEAQKQAIYRQLDQTTQRFNVGLVAITDVQNARSQYDAVLANEVTARNDLDNAVEELRQVTGNYYPELASLNVNGFKTNKPQAVNALLKEAENRNLSLLQARLNQDLAREQIRQAQDGHLPTLSLSASSGVSNTSYSGSKTHNNPQQYQDNDAGQNQIGLNFSLPLYQGGAVTSQVKQAQYNFVGASEQLESAHRSVVQTVRSSFNNVNASISSINAYKQAVVSAQSSLDAMEAGYSVGTRTIVDVLDATTTLYNAKQQLSNARYNYLINELNIKSALGTLNEQDLVALNNTLGKPIPTSADSVAPENPQQDATADGYGNTTAAVKPASARTTQSSGSNPFRQ

SEQ ID NO: 394 - protein, 489 aa, Klebsiella sp.
MKKLLPILIGLSLTGFSAMSQAENLLQVYQQARISNPDLRKSAADRDAAFEKINEARSPLLPQLGLGADYTYTNGYRDSNGVNSNVTSGSLQLTQVLFDMSKWRALTLQEKTAGIQDVTYQTDQQTLILNTATAYFKVLAAIDTLSYTEAQKQAIYRQLDQTTQRFNVGLVAITDVQNARSQYDAVLANEVTARNDLDNAVEGLRQVTGNYYPELASLNVNGFKTNKPQAVNALLKEAENRNLSLLQARLSQDLAREQIRQAQDGHLPTLNLSASTGVSNTRYNGSKTNTPLAYNDSDNGQNQIGLNFSLPLYQGGAVTSQVKQAQYNFVGASEQLESAHRSVVQTVRSSFNNVNASISSINAYKQAVVSAQSSLDAMEAGYSVGTRTIVDVLDATTTLYNAKQQLSNARYNYLINELNIKSALGTLNEQDLVALNNTLGKPISTSADSVAPENPQQDATADGYGNTTAAVKPVSARTTQSSGSNPFRQ

SEQ ID NO: 395 - protein, 490 aa, Klebsiella sp.
MKKLLPILIGLSLTGFSAMSQAENLLQVYQQARISNPDLRKSAADRDAAFEKINEARSPLLPQLGLGADYTYNNGYRDSNGINSNVTSGSLQLTQVLFDMSKWRALTLQEKTAGIQDVTYQTDQQTLILNTATAYFKVLAAIDTLSYTEAQKQAIYRQLDQTTQRFNVGLVAITDVQNARSQYDAVLANEVTARNDLDNAVEELRQVTGNYYPELASLNVNGFKTNKPQAVNALLKEAENRNLSLLQARLNQDLAREQIRQAQDGHLPTLSLSASSGISNTSYSGSKTHNNPQQYQDNDAGQNQIGLNFSLPLYQGGAVTSQVKQAQYNFVGASEQLESAHRSVVQTVRSSFNNVNASISSINAYKQAVVSAQSSLDAMEAGYSVGTRTIVDVLDATTTLYNAKQQLSNARYNYLINELNIKSALGTLNEQDLVALNNTLGKPISTSADSVAPENPQQDATADGYGNTTAAVKPVSARTNQSSGSNPFRQ

SEQ ID NO: 396 - protein, 489 aa, Klebsiella sp.
MKKLLPILIGLSLTGFSAMSQAENLLQVYQQARISNPDLRKSAADRDAAFEKINEARSPLLPQLGLGADYTYTNGYRDSNGVNSNVTSGSLQLTQVLFDMSKWRALTLQEKTAGIQDVTYQTDQQTLILNTATAYFKVLAAIDTLSYTEAQKQAIYRQLDQTTQRFNVGLVAITDVQNARSQYDAVLANEVTARNDLDNAVEGLRQVTGNYYPELASLNVNGFKTNKPQAVNALLKEAENRNLSLLQARLSQDLAREQIRQAQDGHLPTLNLSASTGVSNTRYNGSKTNTPLAYNDSDVGQNQIGLNFSLPLYQGGAVTSQVKQAQYNFVGASEQLESAHRSVVQTVRSSFNNVNASISSINAYKQAVVSAQSSLDAMEAGYSVGTRTIVDVLDATTTLYNAKQQLSNARYNYLINELNIKSALGTLNEQDLVALNNTLGKPIPTSADSVAPENPQQDATADGYGNTTAAVKPASARTTQSSGSNPFRQ

SEQ ID NO: 397 - protein, 482 aa, Klebsiella sp.
MKKLFPILIGLGLTGFSAMSQAENLLQVYQQARISNPDLRKSAADRDAAFEKINEARSPLLPQLGLGADYTYTSGFRDYKDQNSNVTSGSLQLTQVLFDMSKWRALTLQEKNAGIQDVTYQTDQQTLILNTATAYFKVLAAIDTLSFTEAQKQAIYRQLDQTTQRFNVGLVAITDVQNARSQYDAVLANEVTARNSLDNAVEELRQVTGNYYPELASLNVDGFKTTKPSAVNALLKEAENRNLSLLQARLSQDLAREQIRQAQDGHLPTLNLNASSGVSNNRYSGSKSLAQDADVGQNKIGLSFSLPLYQGGMVNSQVKQAQYNFVGASEQLESAHRSVVQTVRSSFNNVNASISSINAYKQAVVSAQSSLDAMEAGYSVGTRTIVDVLNATTTLYEAKQQLSNTRYNYLINELNIKSALGTLNEQDLVALNNTLGKPVPTSPDSVAPENPQQDASADGYSNTAAAKPASARSTSGSNPFRQ

SEQ ID NO: 398 - protein, 484 aa, Klebsiella aerogenes
MKKLFPILIGLGLTGFSAMSQAENLLQVYQQARISNPDLRKSAADRDAAFEKINEARSPLLPQLGLGADYTYTSGFRDYKDRNSNVTSGSLQLTQTLFDMSKWRALTLQEKTAGIQDVTYQTDQQKLILNTATAYFKVLAAIDTLSYTEAQKQAIYRQLDQTTQRFNVGLVAITDVQNARSQYDAVLANEVTARNDLDNAVEELRQVTGNYYPELASLNVNGFKTSKPQAVNALLKEAENRNLSLLQARLNQDLAREQIRQAQDGHLPTLDLNASTSVSNNRYSGSKNISPDADIGQNTVGLNFTLPLYQGGMVNSQVKQAQYNFVGASEQLESAHRSVVQTVRSSFNNVNASISSINAYKQAVVSAQSSLDAMEAGYSVGTRTIVDVLDATTTLYNAKQQLSNARYNYLINELNIKSALGTLNEQDLIALNNTLGKAIPTSPDSVAPENPQQDAAADGYANTASAQPAAARTTKTSGSNPFSH

SEQ ID NO: 399 - protein, 484 aa, Klebsiella huaxiensis
MKKLFPILIGLGLTGFSAMSQAENLMQVYQQARISNPDLRKSAADRDAAFEKINEARSPLLPQLGLGADYTYTSGFRDSKDQNSNVTSGALQLTQVLFDMSKWHALTLQEKTAGIQDVTYQTDQQTLILNTATAYFKVLAAIDTLSYTEAQKQAIYRQLDQTTQRFNVGLVAITDVQNARSQYDAVLANEVTARNELDNAVEELRQVTGNYYPELASLNVDGFKTNKPSAVNALLKEAENRNLSLLQARLSQDLAREQIRQAQDGHMPTLNLNASSSVSNSSYSGSKSTTPDRDIGQNQIGLSFNLPLYQGGMVNSQVKQAQYNFVGASEQLESAHRSVVQTVRSSFNNVNASISSINAYKQAVVSAQSSLDAMEAGYSVGTRTIVDVLDATTTLYNAKQQLSNARYNYLINQLNIKSALGTLNEQDLVALNNTLGKPIPTSPDSVAPENPQQDASADGYSNTAAAKPASARTTSTSGSNPFRQ

SEQ ID NO: 400 - protein, 490 aa, Klebsiella pneumoniae
MKKLLPILIGLSLTGFSAMSQAENLLQVYQQARISNPDLRKSAADRDAAFEKINEARSPLLPQLGLGADYTYNNGYRDSNGINSNVTSGSLQLTQVLFDMSKWRALTLQEKTAGIQDVTYQTDQQTLILNTATAYFKVLAAIDTLSYTEAQKQAIYRQLDQTTQRFNVGLVAITDVQNARSQYDAVLANEVTARNDLDNAVEELRQVTGNYYPELASLNVNGFKTNKPQAVNALLKEAENRNLSLLQARLNQDLAREQIRQAQDGHLPTLSLSASSGISNTSYSGSKTHNNPQQYQDNDAGQNQIGLNFSLPLYQGGAVTSQVKQAQYNFVGASEQLESAHRSVVQTVRSSFNNVNASISSINAYKQAVVSAQSSLDAMEAGYSVGTRTIVDVLDATTTLYNAKQQLSNARYNYLINELNIKSALGTLNEQDLVALNNTLGKPISTSADSVAPENPQQDATADGYGNTTAAVKPASARTNQSSGSNPFRQ

SEQ ID NO: 401 - protein, 489 aa, Klebsiella pneumoniae
MKKLLPILIGLSLTGFSAMSQAENLLQVYQQARISNPDLRKSAADRDAAFEKINEARSPLLPQLGLGADYTYTNGYRDSNGVNSNVTSGSLQLTQVLFDMSKWRALTLQEKTAGIQDVTYQTDQQTLILNTATAYFKVLAAIDTLSYTEAQKQAIYRQLDQTTQRFNVGLVAITDVQNARSQYDAVLANEVTARNDLDNAVEGLRQVTGNYYPELASLNVNGFKTNKPQAVNALLKEAENRNLSLLQARLSQDLAREQIRQAQDGHLPTLNLSASTGVSNTRYNGSKTNTPLAYNDSDNGQNQIGLNFSLPLYQGGAVTSQVKQAQYNFVGASEQLESAHRSVVQTVRSSFNNVNASISSINAYKQAVVSAQSSLDAMEAGYSVGTRTIVDVLDATTTLYNAKQQLSNARYNYLINELNIKSALGTLNEQDLVALNNTLGKPISTSADSVAPENPQQDATADGYGNTTAAVKPASARTTQSSGSNPFRQ

SEQ ID NO: 402 - protein, 483 aa, Kosakonia sp.
MKKLLSLLIGLSLTGFSTMSQAENLLQVYQQARLSNPDLRKSAADRDAAFEKINEARSPLLPQLGLGADYTYTNGFRDSNGVDSNVTSGSLQLTQTIFDMSKWRALTLQEKTAGIQDVTWQTDQQTLILNTATAYFNVLSAIDALSYTEAQKQAIYRQLDQTTQRFNVGLVAITDVQNARSQYDSVLANEVTARNNLDNALESLRQVTGNYYPELASLNVDGFKTDKPKPVNALLKEAESRNLTLLQARLSQDLAREQIRQAQDGHLPTLNLTASTSVSNTKYSGSATRGNAVSSSSYADRDIGQNSVGLSFSLPLYSGGSVTSQVKQAQYNFVGASEQLESAHRSVVQNVRSSFNNVNASISSINAYKQAVVSAQSSLDAMEAGYSVGTRTIVDVLDATTTLYNAKQQLSSARYNYLINELNIKSALGTLNEQDLQALNNSLGKNVSTTPESVAPENPQQDASVDNFNGNVQPAAARTSVKR

SEQ ID NO: 403 - protein, 483 aa, Kosakonia oryzae
MKKLLSLLIGLSLTGFSTMSQAENLLQVYQQARLSNPDLRKSAADRDAAFEKINEARSPLLPQLGLGADYTYTNGFRDSNGVDSNVTSGSLQLTQTIFDMSKWRALTLQEKTAGIQDVTWQTDQQTLILNTATAYFNVLSAIDALSYTEAQKQAIYRQLDQTTQRFNVGLVAITDVQNARSQYDSVLANEVTARNNLDNALESLRQITGNYYPELASLNVDGFKTDKPKPVNALLKEAESRNLTLLQARLSQDLAREQIRQAQDGHLPTLNLTASTSVSNTKYSGSATRGNPVASSSYADRDIGQNSVGLSFSLPLYSGGSVTSQVKQAQYNFVGASEQLESAHRSVVQNVRSSFNNVNASISSINAYKQAVVSAQSSLDAMEAGYSVGTRTIVDVLDATTTLYNAKQQLSSARYNYLINELNIKSALGTLNEQDLQALNNSLGKNVSTAPESVAPENPQQDASVDNFRGNVQPAAARTSVKR

SEQ ID NO: 404 - protein, 447 aa, Legionella geestiana
MLSVVGLSMPLHAADLMDVYQQALESDPAFKQAYDTYLSTSEAIPQAISALRPQVAVGAQAGRSYEDASVGATAFDTTYFNNQWQINASQAVFNFQAWSQVLQARAAVKAAQATFNDAAQSLILRTASAYLDILLARDTLSFAEAKKRANKRQLDQAQQRFKVGLDAITSVYEAQAAYDQSVAEVISARNNQINQNENLRKLTNHVYEFLAPLRDSRIPLIRPEPDNVDNWIDTGIRQNYKLYSAKYSLEAARDNIRVQSGTGWPTLAIQGNAQQVHNQTTQSSFFVPSRATSANIALALNFPMFQGGLVASKTRQAQFDFQASSEQLEQIYRDVVVNSRIAFNTIIDGISKVKADRQTIISQTNSLESTEAQFQVGTRTMVDVVNAQQRLFDAQRQLASDQYNLIRAMLQLKYLAGTLNVNDLQEVNSWLDTTRVNGFPPAKPTKP

SEQ ID NO: 405 - protein, 457 aa, Legionella longbeachae
MKKFLFCTLMTLGLLQQAFATDLMDIYHQALENDPLFKNAYDTYMANAEVLPQARSALRPQVGITGQASRNFFHVNDGFFTIQDTYYNSRTWQISASQAIFNFQAWAKVQQAKASVKAAQAVFNDAAQDLILRTAKAYFDALYAQDTLNFAEAKKRANKRQYDQAQQRFQVGLDPITSVYEAKAAYDQSVATVIASRNNLINQNENLRKLTNHVYDSMAPLRNGEIPLIKPEPNYADQWVDTGLKQNYKLLSAKYNLEVARENVKALSAGNWPVISLQGNTAETSNSLNTTTDNNPLLPVNQKQSTVGLALNFPVYQGGLVRSQTRQAKHNFQAASEQVEQAYRNVVVNSRIAFNTIIDGISKVKADRQSVISQKNSLDSTEAQFEIGTRTMVDVVNAQQRLFEAQNLLANDQYNLINAVLTLKYQAGTLNANDLELINSWLQTTRVKGFPQTGNTK

SEQ ID NO: 406 - protein, 455 aa, Legionella pneumophila
MRKSLFCWILTLGVSTHVFATDLMDIYQQALENDTIFKEAYDTYMSSTEAIPQARAALYPQVGLGSQAGRNYQDAVAGAFSANQYYGSYLWQVNASQALFNYQAWAKVAQAKASVKAAQATFNDAAQNLILRTAKAYFDVLFAKDTLDFAEAKKRANKRQYDQATQRFQVGLDAITSVYEAKAAYDQSIATVIAARNNQINQSENLRKLTNHVYETLAPLKDSKIPLVKPEPNDVNQWIDTGLKQNYKLYAAKYNLEVAKDNVKAISAGNWPVFSLQSNASQVHNNASGNTVFIPSKQTQANIAIAMNFPVFQGGLVQAQTRQAQYGFQSTSEKLEQTYRDVIVNSRIAFNTITDGISKVKADRQTVISVQNSLQSTEAQFEVGTRTMVDVVNAQQRLFEAQEQLARDQYDLINSILTLKYLAGTLNVNDLEQINSWLATTRVNGFSPVDNKTSK

SEQ ID NO: 407 - protein, 455 aa, Legionella pneumophila
MRKSLFCWILTLGVSTHVFATDLMDIYQQALENDTIFKEAYDTYMSSTEAIPQARAALYPQVGLGSQAGRNYQDAVAGAFSANQYYGSYLWQVNASQALFNYQAWAKVAQAKASVKAAQATFNDAAQNLILRTAKAYFDVLFAKDTLDFAEAKKRANKRQYDQATQRFQVGLDAITSVYEAKAAYDQSIATVIAARNNQINQSENLRKLTNHVYETLAPLKDSKIPLVKPEPNDVNQWIDTGLKQNYKLYAAKYNLEVAKDNVKAISAGNWPVFSLQSNASQVHNNASGNTVFIPSKQTQANIAIAMNFPVFQGGLVQAQTRQAQYGFQSTSEKLEQTYRDVVVNSRIAFNTITDGISKVKADRQTVISVQNSLQSTEAQFEVGTRTMVDVVNAQQRLFEAQEQLARDQYDLINSILTLKYLAGTLNVNDLEQINSWLATTRVNGFSPVDNKTSK

SEQ ID NO: 408 - protein, 457 aa, Legionella sainthelensi
MKKLLFCSLMTLGLLPQAFATDLMDIYHQALENDPLFKNAYDTYMANAEALPQARSALLPQVGITGQATRNFFHVNDGFFTIQDTYYNSRTWQVSASQAIFNFQAWSKVQQAKASVKAAQAIFNNAAQDLILRTAKAYFDSLYAQDTLNFAEAKKRANKRQYDQAQQRFQVGLDPITSVYEAKAAYDQSVATVIASRNNLVNQNENLRKLTNHVYDSIAPIRDGKIPLIKPEPNYADQWVDTGLKQNYKLLSAKYNLEVARENVKALSAGNWPVISVQGNTTETSNSLNTTSDNNPLLPVNQKQSSVGLALNFPVYQGGLVRSQTRQAKHNFQAASEQVEQAYREVVVNSRIAFNTITDGISKVKADRQSVISQKNSLDSTEAQFEIGTRTMVDVVNAQQRLFEAQNLLANDQYNLINAVLTLKYQAGTLNANDLELINSWLETTRVNGFPTTGNTK

SEQ ID NO: 409 - protein, 496 aa, Lelliottia amnigena
MKKLLPILIGLSLTGFSAMSQAENLLQVYQQARLGNPELRKSAADRDAAFEKINEARSPLLPQLGLGADYTYSNGFRDANGVNSNATSASLQLTQTLFDMSKWRALTLQEKSAGIQDVTYQTDQQTLILNTATAYFNVLSAIDSLSYTEAQKQAIYRQLDQTTQRFNVGLVAITDVQNARSQYDSVLANEVTARNNLDNALESLRQVTGNYYPELASLNVDNFKTDKPQAVNALLKEAENRNLALLQARLSQDLAREQIRQAQDGHLPTLGLTASTGVSDTTYSGSKTNSAQYDDSNMGQNKVGLTFSMPLYQGGMVNSQVKQAQYNFVGASEQLETAHRNVVQTVRSSFNNVNASISSINAYKQAVVSAQSSLDAMEAGYSVGTRTIVDVLDATTTLYNAKQQLSSARYNYLINQLNIKSALGTLNEQDLQMLNNTLGKPVSTAPENVAPENPQQDAAVDNFNGNGNADMPAAQPAAARTTTAAPASSGTNPFRH

SEQ ID NO: 410 - protein, 450 aa, Limnobaculum parvum
MKKLLPLIIGLSLVGLSTSSYAENLIQVYQQAKSNNPDLRKSESDRDAAYEKISESRGALLPQLGLNAGYNINRGYRDNSGSKSDVTNGTLQLSQTIFDMSLWRQLNITEKQAGVQDVSYQAQQQTLILDTATAYFNVLRSLDILSTTEAQKDALYRQLDQARQRFNVGLVAVTDVQNAQAQYDQTLADEVTARNNLENSLEVLRQITGMFYPELSSLDTARFSTNRPNNVNTLLQEAESRNLNLLTARLAQDLSREQVKLQETGHMPTVAFTASSGLSNNDYRGTSTIGPEDSINGNTTAGFTLSFPLFSGGQTSSRVKQAQYNYIGASEALESAHRSVVQIVRSSYNNVSASISSVNAYKQTVVSAQSSLEATQAGYDVGTRTIVDVLNATTTLYNAKQQLSNARYDYLINQLNIKYALGTLNENDLDLLNKTLGKPNSTIPVINVKE

SEQ ID NO: 411 - protein, 459 aa, Luteimonas yindakuii
MLRRPLALALSLVLLPASAFAQDLVQTYELARAGDPQLSIAESNREISREGAVQARAALLPQINGSADLSRRYVAGVDGDQRSRSVGASLDQTVFDLGRISNLRAQRQLDTAAGFDLQAANQTLITRTSAAYFNVLVAIETLAAAEAAEQALQKQFDFASRRLEVGLAPITDVHEARAQYDAARAQVILSRNALEDAYQALAEITGVPVRNLRALPEDFQPEVPAGGDADSWVNAALGQNPSLRAAEYAVRAAEIGITGARAGHLPRLSLGAGYNNQAYWGDLAGVNDPGNPTGARRNEGHSVGLTLTVPIFSGGATQSGVREAIARRDIAEDQLTQTRRALERNARNAYQTVVAGVSEVEARRLAVLSARSAYDASQVGLEVGTRTVVDVLINQQTLFQAEQQFALARYNFLQNRLLLEQAVGTLDINDLQEINRLLTVEADSPSTAPTAPASSPQQQ

SEQ ID NO: 412 - protein, 474 aa, Lysobacter arenosi
MIRRPLVIALATAMGLSALMPATASAEDLLQTYELARDSDPQLSASESSRLAVKEGAVQTRAAMLPQIDGSASINRSENHNRGNLAVGSDGSISGGNSQIDTTTRSSEVRLSQMIYDRGNFTRHKSQKALSEASDFQLAAAGNDLITRTSAAYFNVLIALESLVAAEAAETALKKQFDYASKRLEVGLAPITDVHEARAQYDSARANTILARNVLEDAYQGLTELTGKDVRGVKGLPKDFQPVLPESRDAEGWVATAIENNPALRAKDLQVQSTEADVETARAGHWPRLYLNGSYGDSTGWGDNTLHDLGITRDIDNRSYGPAVGVTLSVPIFSGGAVQSGVRQALARRDVASDELESQKRALIRNTRNAYQTLVASVSEVEARRLALVSAKSAYDASQVGLEVGTRTVLDVLQNQNNLFRAQLEYARARYNHLQNRLLLEQAAGTLDIADVQDINRLLTLDTESYVAPGDVQG

SEQ ID NO: 413 - protein, 467 aa, Marinobacter sp.
MKKRLLPGLITLLAAQPAFSLDLMETYEKALSYDSGIASAMAQFQAQQATSDVSKSALLPKISAVGSASYTRFKPRNIPGGANADELTRQLFAGDSYRTYRYGVELTQPLFRAQDWFSYEASQFQTEAAQAQYNLAQQQLILDVATAYFNVLRAQDTVTTARATEAAIQRQYEQAQERFDVGLIAITEVYEARASYDDSKSQRIAAENQLNVAREQLARLTGEYAEDLENLRENFPLGRPEPMDPSAWENTALEQNWVIQSALYQLNANEANLKVAKSGHLPTLDLFASYGKSELDGAKNTQQEGTQGVIGLELNVPLYMGGGTQAGVRQQRSLVTAAQEDLNTTRRDVRVNTRSLYLTVNNNVETASALEQTIISRRSALDATRAGYDVGTRNIVEVLDAERAYYVALRDYANARYDYVINTLQLKQAAGTLSPQDLIELNNWLSANARGIEALAEEDLTLDDPTQ

SEQ ID NO: 414 - protein, 439 aa, Methylomonas sp.
MTKLLPTFFVALTWGSPAFCQDLLETYQLAKANDPEFKSSDINKLATAEIKSQSIAQMLPNISFNANSSRNRLESTSFLGTTLQHYWDHKLGFNLKQPVFHWDHWVQLDQADNKIAQAEAQFQAKQQSLIRRTAEAYFNILAAQDNLEFADSEKKSIEKQLEQAKQRFDVGIIAITDVYEAQAGYDRALASEIEAQNQLDNSKEALREIIGENAADLNSLQPQIPLSPPAPEDLSSWSNAAENNNFSIVAQLNQAEYVRKNVELQQSKHLPTLDIVAQYGDQDTGNRYGLRGDNESVGLQLNLPLFEGGGTSSRSRQAAYEYEAAKEDLTKVKRSINRGVKDAFRGVVSSISRVKALDATSKSAEMAVEAAEAGFEVGTRTMVDVLTEQRNLYKAKSDYARSRYDYLINGIKLKEAAGSLNEQDLEQINQYLQGTLSKN

SEQ ID NO: 415 - protein, 454 aa, Morganella sp.
MKKLLSLLIAVSVAGFSSVTQAEDLLQVYQKAKETNPTLRQTQAERNQAFEKINEARSPLLPQLGLSGGYDYNTGYRDNSNQSNNALNAQLKLTQTIFDMSKWRQLNLQEQTAGISDITFQSAQQQLIVDTAVAYFNVLRALDTLSFVQAQKEAVYRQLDQTTQRFNVGLVAITDVQNARANYDSVLAQEVAGRNDVENAIEALRQVSGIYYSELASLDVNRFKTNAPDAIEALLKEAQERNLNLLGATLSRNLAKEQISLAESGHYPTVNLSAGTGVTNTDYSGNGYPNGRPNNSYAGQSTIGVNVSIPLYTGGAVSSQVDQAQYGYTAASEKLEATLRQVVQTTRSSYNNINASISSVNAYKQVVTSAESSLDATEAGYQVGTRTIVDVLNATTTLYEAKKSLSNARYDYLINQLYIGQARGTLNEDDILRLNQVLGKTISTSPASVAPTTR

SEQ ID NO: 416 - protein, 447 aa, Moritella yayanosii
MKFNKILLACGLAFFAHSASADNLQQIFQQALTKDPLYLEAQANRDAALEKITEQEAANLPQIGLSADLGYGITSDYTTANTTSNGNALTGSVGIGLTQSLYEESNFINVSQAAKQAEQSELAVQAELQNLILRVANAYFSVLGANDTLEFSNRNKDAVERQLEQTTQRYNVGLTDKTDVLEAQSSFDLSVAEVINAENTLANSYESLTEITGLTHINIATLNTARFSPTAPDGQRDNWLATANNQNLAIQIQRIAKQIAEGNVELASSADNMSLNLVANAGAQYTGYGDDYNNGGAGLESTVSSANVGIEFSMPLYTGGAVTSAKKQAAYQVTASQEQLTRASREVQTKIRTFYNNVTAGLSSIKAYEQTVKSSQSALEATQAGFGVGTRTIIDVLDATKTLYQSKQSLSASRYNYIISMLQLKQAAGTLNAEDIDLVNAGLETSN

SEQ ID NO: 417 - protein, 489 aa, Pantoea sp.
MKKLLPLFIGLSLGGFSVASQAENLLQVYQQARLSNPDLRSSAATRDAAFEKINEARSPLLPQLGLGADYSYANGYRDSSGIHSNTTSGTLQLTQTIFDMSKWRALTLQEKTAGIQDVTYQVAQQDLILNTATAYFNVLKAIDTLSYVEAQKMSIYRELDQTTQRFNVGLVAITDVQNARAQYDSVLANEVTARNNLDNSVESLRQITGMDYLALASLNIDRFKTTKPDDVNALLKQAESRNLNLLSARLNQDLAREQIRSAESGHLPTLDLTASTGLSNSKYGGSRAEQSANSTDSITGNSKVGLSFTLPLYSGGSVTSQVKQAQYNFVSASEQLEAAHRSAVQTVRSSYNNVNASASSIAAYKQAVVSAQSSLDAMEAGYQVGTRTIVDVLNATSTLYNAKQQLSDARYSYLINQLNISYALGTLNEQSLGILNSQLGKEISTSPETVQPQNVQQVLGEDHIASSSETATAPVRPAAHKSSRNPFAN

SEQ ID NO: 418 - protein, 489 aa, Pantoea sp.
MKKLLPLFIGLSLGGFSVASQAENLLQVYQQARLSNPDLRSSAATRDAAFEKINEARSPLLPQLGLGADYSYANGYRDSSGIHSNTTSGTLQLTQTIFDMSKWRALTLQEKTAGIQDVTYQVAQQDLILNTATAYFNVLKAIDTLSYVEAQKMSIYRELDQTTQRFNVGLVAITDVQNARAQYDSVLANEVTARNNLDNSVESLRQITGMDYLALASLNIDRFKTTKPDNVNALLKQAESRNLNLLSARLNQDLAREQIRSAESGHLPTLDLTASTGLSNSKYGGSRAEQSANSTDSITGNSKVGLSFTLPLYSGGSVTSQVKQAQYNFVSASEQLEAAHRSAVQTVRSSYNNVNASASSIAAYKQAVVSAQSSLDAMEAGYQVGTRTIVDVLNATSTLYNAKQQLSDARYSYLINQLNISYALGTLNEQSLGILNSQLGKEISTSPETVQPQNVQQVLGEDHIASSSETATAPVRPAAHKSSRNPFAN

SEQ ID NO: 419 - protein, 489 aa, Pantoea ananatis
MKKLLPLFIGLSLGGFSVASQAENLLQVYQQARLSNPDLRSSAATRDAAFEKINEARSPLLPQLGLGADYSYANGYRDSSGIHSNTTSGTLQLTQTIFDMSKWRALTLQEKTAGIQDVTYQVAQQDLILNTATAYFNVLKAIDTLSYVEAQKMSIYRELDQTTQRFNVGLVAITDVQNARAQYDSVLANEVTARNNLDNSVESLRQITGMDYLALASLNIDRFKTTKPDDVNALLKQAESRNLNLLSARLNQDLAREQIRSAESGHLPTLDLTASTGLSNSKYGGSRAEQSANSTDSITGNSKVGLSFTLPLYSGGSVTSQVKQAQYNFVSASEQLEAAHRSAVQTVRSSYNNVNASASSIAAYKQAVVSAQSSLDAMEAGYQVGTRTIVDVLNATSTLYNAKQQLSDARYSYLINQLNISYALGTLNEQSLGILNSQLGKEISTSPETVQPQNVQQVLGEDHIASSSETATAPVRPSAHKSSRNPFAN

SEQ ID NO: 420 - protein, 490 aa, Pantoea sp.
MKRLLPLFIGLSLGGFSLASQAENLLQVYQQARLSNPTLRASAADRDAAFEKINEARSPLLPQLGLGADYTYNNGFRDSSGLHSNTTSGSLQLTQTIFDMSKWRALTLQEKTAGIQDVTYQIAQQDLILNTATAYFNVLNAIDTLSYTEAQKQSIYRELDQTTQRFNVGLVAITDVQNARAQYDSVLASEVTARNTLDNAVEALRQITGMDYLSLAELNIDRFKTTRPDAVSSLLKEAESRNLSLLSARLSQDLAREQIRSAETGHMPTLDLTASTGLSNSKYGGSRANQSSSTSDSITGSNQVGLSFSLPLYSGGSVTSQVKQAQYSFVSASEQLESAHRSAIQTVRSSFNNVNASISSIDAYKQAVVSSQSSLDAMEAGYQVGTRTIVDVLDATTTLYNAKQQLADARYNYMINQLNIKYALGTLNEQDLQLLNAQLGKETSTSPETVAPENTQQASRVDNGPKASSASAAAKARPAAARNTSNPFSN

SEQ ID NO: 421 - protein, 488 aa, Pantoea vagans
MKKLLPFFIGLSLGGFSFASQAEDLLQVYQQARLSNPDLRSAAADRDSAFEKINEARSPLLPQLGLGADYTYNNGYRDSSGLHSNTTSGTLQLTQTIFDMSKWRALTLQEKTAGIQDVTYQVAQQDLILNTATAYFNVLKAIDTLSYTEAQKQSIYRELDQTTQRFNVGLVAITDVQNARAQYDSVLANEVTARNNLDNNVETLRQITGMDYMSLASLSIDRFRTDKPEAVSALLKQAESRNLSLLSARLNQDLAREQIRSAETGHMPTLDLTASTGMSNSKYGGSRANQSTGSTDSITGSNQVGLSFSLPLYSGGSVTSQVKQAQYSFVSASEQLESAHRSAVQTVRSSFNNVNASISSINAYKQAVVSAQSSLDASEAGYQVGTRTIVDVLDATTVLYNAKQQLSDARYSYLINQLNISYALGTLNEQSLQKLNSQLGKEIPTSPETVAPENAQQTARVDSGPVATGSSEAARPAARHSSGNPFGN

SEQ ID NO: 422 - protein, 463 aa, Pectobacterium sp.
MKKLLPLLIGLSLGGFSAMSQAENLLQVYQQAKSTNPDLRSSAATRDAAFEKINESRSPLLPQLGINAGYTYNKGYRDSNGVNNNEKSATLQLTQTLFDMSKWRALTLQEKQAGIEDVTYQTAQQNLMLNTATAYFNVLRAIDSLSYINAQKQAIYRQLDQTTQRFNVGLVAITDVQNARAQYDSVLANEVLTRNTLDNALESLRQITGNFYPQLAGLNIERFSTQKPEAVNNLLKEAENRNLNLLSARLSQDLAREQIRSAETGYMPTLDLTASTGVSDTRYSGSRTNSGNFNDTDAGQHRVGINFTLPLYSGGATNSQVKQAQHSYVSASELLESAHRSVIQTVRSSFNNISASISSINAYKQAEVSAQSSLDAMEAGYQVGTRTIVDVLDATTTLYNAKQQLSSARYDYLINQLNIKSAQGTLNEADLQALNASLGQPVSTTPTVTDNTAPLATTASAQR

SEQ ID NO: 423 - protein, 463 aa, Pectobacterium parmentieri
MKKLLPLLIGLSLGGFSAMSQAENLLQVYQQAKSTNPDLRSTAATRDAAFEKINEARSTLLPQLGINAEYTYNKGYRDSNGVNNNEKSASLQLTQTLFDMSRWRALTLQEKQAGIEDVVYQTAQQNLMLNTATAYFNVLRAIDSLSYISAQKQAIYRQLDQTTQRFNVGLVAITDVQNARAQYDSVLANEVLTRNTLDNALESLRQITGNFYPQLAGLNITHFSTQKPEAVNNLLKEAENRNLNLLSARLSQDLAREQIRSSETGYMPTLGLTASTGVSDTRYSGSRTNSGNFNDTDAGQHRVGINFTLPLYNGGITNSQVKQAQHSYVSASEQLESAHRSVIQVVRSSFNNISASISSINAYKQAEVSAQSSLDAMEAGYQVGTRTIVDVLDATTTLYNAKQQLSSARYDYLINQLNIKSAQGTLNESDLQALNASLGQPVSTTPTITDDSAPQTTTASAQR

SEQ ID NO: 424 - protein, 464 aa, Pectobacterium versatile
MKKLLPLLIGLSLGGFSAMSQAENLLQVYQQAKSTNPDLRSSAATRDAAFEKINQARSPLLPQLGLGADYTYNRGYRDSKGVDSNVKGASLQLTQTLFDMSKWRALTLQEKQAGIEDVTYQTAQQNLMLNTATAYFNVLRAIDSLSYINAQKQAIYRQLDQTTQRFNVGLVAITDVQNARAQYDSVLANEVLTRNTLDNALESLRQITGNFYPQLAGLNIERFSTQKPEAVNNLLKEAENRNLNLLSARLSQDLAREQIRSAETGYMPTLDLTASTGVSDTRYSGSRTQNSNSFNDSDAGQHKVGINFTLPLYSGGATNSQVKQAQHSYVSSSELLESAHRSVIQTVRSSFNNISASISSINAYKQAEVSAQSSLDAMEAGYQVGTRTIVDVLDATTTLYNAKQQLSSARYDYLINQLNIKSAQGTLSETDLQALNASLGQPVSTTPAVTDNSAPLATTASAQR

SEQ ID NO: 425 - protein, 434 aa, Photobacterium damselae
MKKLLPLFISVAFGGLSQSALADNLAEIYQQAKQNDPQLLKAAADKDAAFEAINSARSSLLPQINLSAGYKGNFMDADTPAVDNHGFNTGLQLSQSIYNRSSWVNLDISEQQARQSDAAYAAAQQALIMRVSKAYFDVLKAQDDLTFVRAEKAAVGRQLEQMKQRFDVGLSAITDVYDAQAQYDSVLASEILKENALTNSYEELREITGQAHKNLDILDTKSFSASNPSKPVNDLVKGAETENLTLLTYRIAQDVARNKISLAESGHLPTLSFDTGYSYDQTRMGNDDGSLTAGVTMNLPVYTGGRTTSEVKQAQFAYVSASEQLEQQYRSVVKDVRAYYNNINSSIASLRAYQQSVVSAKSALEATEAGFDVGTRTIVDVLNSTQSLYNANSQLADARYNYILSQLQLKQAVGTLSEQDILDINQGLKAVAKK

SEQ ID NO: 426 - protein, 457 aa, Photorhabdus sp.
MKKLLSIFIAMSLTGLSSMSQAEDLLQVYKQAKEGNPELRKSMAERNKAFEKINESRSPLLPQLELNTGYTYNSGYRDQRDTESNTLNAKLGLTQTIFDMSKWRLLNLEEKTAGIADVTYQSSEQKLILDSATAYFKVLRTIDTLSYIEAQKAAIYRQLDQTTQRFNVGLVAITDVQNARANYDDVLAQEVASRNELDNALEALRQVTGIYYPRLASLNIDHFKTQKPEAVDILLKEAEKRNLSLLSARLGQDLAREQIKAAQTGHMPTIDLGASTNVSNSQNHAPGRSNNYSGENSVGLTLRLPIYSGGATSSRVEQAQYGFVSASEQLESTYRNVVQTVRSSFNNISASISSIDAYKQLVVSAQSSLDAMEAGYQVGTRTIVDVLNATTKLYDAKQKLSNARYDYLINQLNIQHALGTLNEKDLVALNNSLGKQLSTSANSIVQGTGTPVTSARN

SEQ ID NO: 427 - protein, 465 aa, Proteus sp.
MKKLLSLLVTMSLAGFSTASQAEDLLQVYQKAKDSNPELRKSLAERNQAFEKINEARSPLLPQLGLGASIDYKSGYRDAKNTESNSLGANLTLTQTIFDMSLWRQLTMQEKTAGMSDVTYQTSQQQLILDTATAYFNVLRAIDSLSFIEAQKEQVYRQLDQTTQRFNVGLVAITDVQNARANYDSVLAQEVAGRNELDNALEKLRQVSGVYYINLASLNIARFSTTPPDAIDKLLKDAEERNLSLLSARLGQDLARENIRLAQSGHLPTVNLNASTGVSNSHNHGSALPPETPANSRNSYNGQSSIGLSLSIPLYTGGRTSSQVEQAQYGFTSASEQLESVYRSIVQIARSSYNNISASISSIKAYQQVVVSAQSSLDATEAGYQVGTRTIVDVLNATTTLYDAKQKLSSARYDYLINQLNIQYARGTLNENDLIQLNNALGEEISTSPDNIIRPLTSPVLNTTN

SEQ ID NO: 428 - protein, 458 aa, Providencia sp.
MKKLLPLFIALSFVGLSTNSYAEDLLQVYQKSKESNPDLRKSLAERNQAFEKINESRSPLLPQLGLGAGITYGSGYRDTRNTESNGLNASLKLTQVVFDMSKWRQLSVQEKTAGITDVSYQTSQQQLILDTATAYFNVLKAIDALSYIEANKEAVYRQLDQTTQRFNVGLVAITDVQNARANYDSVLAQEVSGRNDLENAVEKLRQVSGVYYNQLASLNIDRFKTVTPDQVDAILKEAEERNLSLLSARLAQDVSRENIRLAETGHMPTINLDAATSVANTHNHGSNFNGLDRNSYTGQNSIGLTLSLPLYSGGAVSSQVEQAQYGFQGASEQLESVYRNVIQLVRSSYNNVSSSISSINAYKQVVVSAQSSLDAMEAGYQVGTRTIVDVLTATTALYQAKQNLSNARYDYMINQLNIEFARGTLNEEDIARLNASLGKEVSTSPSSIIRNIETPQIR

SEQ ID NO: 429 - protein, 450 aa, Providencia sp.
MNKIIKLFILISSIIIGYNCNAEDLLQVYKITKDNNPTLKKTESERYQAYKKIDEERSSLLPQLKLKANSNYKNNDGTDDNKNIKNTELSLQMTQSIIDISKWKSLSIQEKHAAISDINYQISQQDLLLETAISYFNILKSIDALTYIKAHKLAVYHQLDQTTQRFNVGLTTITDVQNARANYDSIIAQEVLGYNELKNSVEVLRKISGVRYTQLAKLNINKLQLTKPDNITFLLENARIKNLSLLKARLSQEISNDKISLAKSGHLPIVTLDASTAINNTRLDKNSIESQHKINNHSGKNNAALSLNLPIYQGNTVNSQVKQAQYGFEAATEQLESDNRNVIQQVNSSYNNLLSSISSINAYKQLIISTQSSLDATEAGYQVGTRTIVDVLSATAALYQSKKNLSNAQYDYLINQLKMEYLQGSLNENSIQRINKMLGNEISIYEPTMQ

SEQ ID NO: 430 - protein, 446 aa, Pseudoalteromonas sp.
MKKNILAALVSLSCALGTTAANAEDLLQVFEIATANDPTVLKAKAQADAQSYQADTAMAALLPQIGLTMGYTKSDSTSFESVDEKVESDSNQFTRGLSLSQTLFDLGAWNSLDIADKQALQANAQYDSAKQNLIVRVAEGYFNVLSAIDNLEFIKTEKRAIERQLEQTKQRYAVGLTAITDVHEAQAQFDNSVAQEIIANNSVETAREQLREITGKYHAKLDFLNTETFSTTKPARDSSDFLNIAKDKNISLQVAKVTVDIAQDQIKLARAGHYPKLTLSASYGDSLTDADRDGTSLTRQPRADSTSVGLNFSLPLYSGGATVAATDQARAFYVAASQDYEANYRAITRTVITSYNQVVSDIATYKALEQAVVSAQSALKATEAGFEVGTRTIVDVLVSTQNLYNAKRNLADVRYRYVLSTLRLKQAAGTLTSKDLVAINQGLKAV

SEQ ID NO: 431 - protein, 448 aa, Pseudoalteromonas sp.
MKKSILSLFIGVACALSSTASQAEDLLQVYEIATANDPTVLKAKAQADAQKYAQDSALGDLLPSLGFSMSYTDNDGESTLPGQNDASGYTVVDTWSDSITRSITLNQTIFSMATWQNLTIAEKQAMQALTSYEQQQQDLIVRVAQGYFDVLSALDNLEFVQAEKRAIERQLEQTKQRYEVGLTAITDVHEAQAQFDQSVANEIVAQNTVETARETLRTITGKYHAKLDKLNTDSFSTVKPNRQSNDFITLAKEQNLSLQVAKSALDIAQDQIDLAQAGHYPTLSLQASYSDSLGNTEGAELSPRTDGTSIGLSFNVPIYSGGKTVAATDQARALYVAASEDLEASMRNVTRSVITSYNQVMSDVATYRALEQAVVSAESALQATEAGFEVGTRTIVDVLVSTRNLYSAKRNLANLRYQYVLSTLKLKQAAGTLNRSDLEAINKGLVAG

SEQ ID NO: 432 - protein, 451 aa, Pseudoalteromonas sp.
MKKNILAALISLSCALGSTAANAEDLLNVFEIATANDPTVLKAKAQADAQSYESDRAMSVLLPQINLTMGYDKSDATSYQSVGDLASQVQVESDTDQFTRGVSLSQTLFDLGAWNSLGIADKQALQASGQYDAAKQNLIVRVAEGYFNVLSAVDNLEFIEAEKRAIERQLEQTKQRYAVGLTAITDVHEAQAQFDNSVAQEIIASNAVETAREQLREITGKYHAKLDFLNTDTFSTTKPAKLSSDFIKVAENKNISLQVSKVTVDIAKDQIELAKAGHYPKLTLSASYGDSLTDTQRNGVNFDDQPRSDSSSVGLNFSVPLYSGGATVAATNQARAFYVAASQDYETNYRAITRTVITSYNQVVSDIATYKALEQAVVSAQSALKATEAGFEVGTRTIVDVLVSTQNLYNAKRNLADVRYRYVLSTLRLKQAAGTLTSEDLVAINQGLKAA

SEQ ID NO: 433 - protein, 479 aa, Pseudomonas sp.
MLRKLSLVVAVSFASSGLTWAADVPLPTQTGLLNVYQQAVDNNADLAASRADYDARKEAVPQARAGLLPNISGSAQSNNTRTSIDQPRAVATRSGNVYQATLSQPIFRADRWFQLQAAEASNEQAALELSATEQNLILQAAQSYFSVLRAQDNLASTKAEEAAFKRQLDQANERFDVGLSDKTDVLQAQASYDTSRASRILAKRQVDDAFQALVTLTNREYNSIEGIVHTLPVLAPTPNDAKAWVDTAAQQNLNLLASNYAVNAAEETLRQRKAGHAPTLDAVATYQRGDNDALGFSNPNYTGRNYGGDVEQRSIGVQLNIPIYSGGLTSSQVREAYARLSQSEQRRESLRRQVVENTRNLHRAVNTDVEQVQARKQSIISNQSALEATEIGYQVGTRNIVDVLDAQRQLYASVRDYNNTRYDYILDNLRLKQAAGTLSPGDLQDLSRYLKADYNPDKDFLPPDLATAAQKNFERPAKR

SEQ ID NO: 434 - protein, 482 aa, Pseudomonas sp.
MLRRLSLAAAVAAATGVAWAAQPTPLPTKTDLISVYKEAVDNNADLAAAQADYLARKEVVPQARAGLLPQLGAGARVGDTRIAFDERPATVKRNSQVVQATLSQPLFRADRWFQWQAAKETSDQARLEFSATQQDLILRSAETYFTVLRAQDNLATSKAEEAAFKRQLDQANERFDVGLSDKTDVLEAQASYDTARANRLIAEQRVDDAFQALVTLTNRDYSAIEGMRHTLPVVPPAPNDAKAWVDTAVQQNLRLLASNYAVNAAEETLRQRKAGHLPTLDAVAQYQKGDNDALGFANSAANPLVHYGKYVDERSIGLELNIPIYSGGLTSSQVRESYQRLNQSEQSREGQRRQVVQDTRNLHRAVNTDVEQVQARRQAIISNQSSLEATEIGYQVGTRNIVDVLNAQRQLYAAVRDYNNSRYDYILDTLRLKQAAGTLSPADLEALSAYLKQDYDPDKDFLPPDLAKAAAEQLQSKPRQQY

SEQ ID NO: 435 - protein, 482 aa, Pseudomonas sp.
MLRRLSLAAAVAAATGVAWAAQPTPLPTKTDLISVYKEAVDNNADLAAAQADYLARKEVVPQARAGLLPQLGAGGRIGDSRIALDEPAATVKRSSHVVQASLSQPLFRADRWFQWQAAKETSDQAKLEFSATQQDLILRSAETYFTVLRAQDNLATSKAEEAAFKRQLDQANERFDVGLSDKTDVLEAQASYDTARANRLIAEQRVDDAFQALVTLTNRDYSAIEGMRHTLPVVPPAPNDAKAWVDTAVQQNLRLLASNYAVNAAEETLRQRKAGHLPTLDAVAQYQKGDNDALGFANSAANPLVHYGKDVEERTIGLELNIPIYSGGLTSSQVRESYQRLNQSEQSREGQRRQVVQDTRNLHRAVNTDVEQVQARRQAIISNQSSLEATEIGYQVGTRNIVDVLNAQRQLYAAVRDYNNSRYDYILDTLRLKQAAGTLSPADLEALSAYLKQDYDPDKDFLPPDLAKAAAEQLQSKPRQQY

SEQ ID NO: 436 - protein, 479 aa, Pseudomonas sp.
MLRKLSLVVAVSFASSGLTWAAELPMPTQTGLLSVYQQAVDNNADLAASRADYDARKEAVPQARAGLLPNISGSAQNSSTRTSIDQPRAVATRSGNLYQATLSQPIFRADRWFQLQAAEASNEQAALELSATEQNLILQSAQSYFSVLRAQDNLASTKAEEAAFKRQLDQANERFDVGLSDKTDVLQAQASYDTSRASRLIAQRQVDDAFQALVTLTNREYNSIEGIVHTLPVLAPTPNDAKAWVDTAAQQNLNLLASNYAVSAAEETLRQRKAGHAPTLDAVATYQRGDNDALGFSNPNYTGRNYGGDVEQRSIGVQLNIPIYSGGLTSSQVREAYARLSQSEQRRESLRRQVVENTRNLHRAVNTDVEQVQARKQSIISNQSALEATEIGYQVGTRNIVDVLDAQRQLYASVRDYNNTRYDYILDNLRLKQAAGTLNPGDLQDLSRYLKADYNPDKDFLPPDLATAAQKNFERPAKR

SEQ ID NO: 437 - protein, 478 aa, Pseudomonas sp.
MLRKLSLAIAVSWATNAMALDTQAPLSSQNGLVSVYHEAVDNNADLAAARADYDARKEVVPQARAGLLPNLSGGAESSNVRTSIDQPSLTTTRSAVVYQATLAQPIFRADRWFQLKAAESVNEQAALQLSATEQNLILQTAESYFSVLRAQDTLASTKAEEAAFKRQLDQSRERFDVGLSDKTDVLQSQASYDTARANRLLAQRQVADAFEALITLTNRQYNSIEGMRHTLPILPPTPNDAKAWVDTAAQQNLNLLASNFAVAAAEETLKQRKAGHAPTLDAVAQYKTGDNDGFGLSNPNALQQRYSGDVSQRSIGLQLNIPIYSGGLTSSQVRESYSRLNQTEQQRESMRRQVVENTRNLHRAVNTDVEQVQARKQSIISNQSALEATEIGYQVGTRNIVDVLDAQRQLYTSVRDYNNSRYDYILDNLRLKQAAGTLNPGDLEALSRYLKPDYNPDKDFLPPDLAQAAEANMRSPGN

SEQ ID NO: 438 - protein, 482 aa, Pseudomonas sp.
MLRRLSLAAAVAAATGVAWAAQPTPLPTKTDLISVYKEAVDNNADLAAAQADYLARKEVVPQARAGLLPQLGAGARVGDTRIAFDERPATVKRNSQVVQATLSQPLFRADRWFQWQAAKETSDQARLEFSATQQDLILRSAETYFTVLRAQDNLATSKAEEAAFKRQLDQANERFDVGLSDKTDVLEAQASYDTARANRLIAEQRVDDAFQALVTLTNRDYSAIEGMRHTLPVVPPAPNDAKAWVDTAVQQNLRLLASNYAVNAAEETLRQRKAGHLPTLDAVAQYQKGDNDALGFANSAANPLVHYGKYVDERSIGLELNIPIYSGGLTSSQVRESYQRLNQSEQSREGQRRQVVQDTRNLHRAVNTDVEQVQARRQAIISNQSSLEATEIGYQVGTRNIVDVLNAQRQLYAAVRDYNNSRYDYILDTLRLKQAAGTLSPADLEALSAYLKQDYDPDKDFLPPDLAKAASEQLQSKPRQQY

SEQ ID NO: 439 - protein, 479 aa, Pseudomonas sp.
MLRKLSLALAVSCATNGMVWAAEAPLSANTDLVSVYQEAVDNNADLAAARAQYGAQKEVVPQARAGLLPNLTGGADINNVRTQIDTPAATANRDAHSWRATLSQPLFRADRWFQLQAAEATNEQAALQLSATEQNLILQSAENYFAVLRAQDNLASTKAEENAFKRQLDQSNERFDVGLSDKTDVLQSQASYDTARANRILAQRQVEDAFEALITLTNRQYNSIQGIVHTLPVLPPLPNDAKAWVETAGRQNLNLLASNYAVTAAEETLKQRKAGHAPTLDAVAQYEKGDNDALGFSNPNAFGTPYRGDVEQRTIGLRLSIPIYSGGLTSSQVRESYSRLGQTEQQREGLRRQVVENTRNLHRAVNTDVEQVQARRQSIISNQSAVEATEIGYQVGTRNIVDVLDSQRQLYASVRNYNNSRYDYILDNLRLKQAAGTLNPGDLQDLARYLKADYNPDRDFLPPDLAKAAAEQLKARPGY

SEQ ID NO: 440 - protein, 477 aa, Pseudomonas sp.
MLRKLSLAIAVSCASNGVVWAADVPSTVKTDLVSVYQEAVDNNADLAAARADYGARREVVPQARAGLLPNLSAGAEMMNTRTKLDEPSITSNRSGNSWSATLAQPIFRADRWFQLQAAEAVNEQAALELSATEQNLILQTAQNYFAVLRAQDNLAATKAEEAAFKRQLDQSNERFDVGLSDKTDVLQSQASYDTARANRIIAERQVQDAFEALVTLTNREYTSIQGVVHSLPVQVPTPNDAKAWVETAGRQNLNLLATNYAVSAAEETLRQRKAGHAPTLDAVAKYQKGDNDSLGFTNPSQLGVRYSGDVEQTSVGLQLNIPIYSGGLTSSQVREAYQRLSQSEQQRESLRRQVVENTRNLHRAVNTDVEQVQARKQSIISNQSALEATEIGYQVGTRNIVDVLDAQRQLYTSVRDYNNSRYDYILDNLSLKQAAGTLSPQDLQDLKRYLKPDYNPDKDFLPPDLAAAAAKNFERRP

SEQ ID NO: 441 - protein, 476 aa, Pseudomonas sp.
MLRKLSLAVAVSCASNGMAWAAEAPLTTKTDLVSVYQEAVDNNADLAAARAQYGAQKEVVPQARAGLLPNLSAGADMNNTRTKFDEPSMTSTRSGNVYQATLAQPLFRADRWFQLQAAKDINEQASLQLSATEQNLILQSAENYFAVLRAQDNLASTKAEEAAFKRQLDQSNERFDVGLSDKTDVLQSQASYDTARANRILAQRQVDDAFEALITLTNREYNSIQGIVHTLPVLAPTPNDAKAWVDTAAKQNLNLLASNYAVSAAEETLKQRKAGHAPTLDAVAQYKKGDNDALGFSNPNPLTRYGSDVEQRSIGLQLNIPIYSGGLTSSQVRESYSRLSQTEQQRESLRRQVVENTRNLHRAVNTDVEQVQARRQSIISNQSAVEATEIGYQVGTRNIVDVLDAQRQLYTSVRNYNNSRYDYILDNLRLKQAAGTLNPGDLQDLSRYLKADYNPDRDFLPPDLAKAAQEQLRARP

SEQ ID NO: 442 - protein, 501 aa, Pseudomonas sp.
MSKPMNSRQRALAGWLLVVPLLVAPLAQAAPLHRQDLPQDSAQHPPAYVPQQRPQALPSDLWRVYLDARQNNSELAAAQADQAARAEAVPQARAGLLPTLSASADLNATSTSLQQPRQDTRRSGTSYQAVLNQPIFRADRWFTLKAAQAEDQQAQLELAAAEQKLMFDSAQAYFGLLKAQDALAAAKAEEAALKRQQQLAERGLQLGLSDRTDVLQAQAGHDTARANRIVAQKQTDDAFEALDTLTHQQYQAVQGLRHDMPVLLPEPNDARRWVDTAVRQNLTLQASQFALEATQQTLSARKAGHAPTLDAVLRYQTGDNDALGYGNSDIRGNGYGGNVEQRTVGLQLNIPLFSGGQTRSQVREAHQRMNQREYLNDGLRRQVVEQTRNLHRGLNSGVDQVQARRQSIISNQGAVLASQMGFQVGTRNIVDVLEAQRQLYNAVRQYNNSRYDYILDTLRLKQAVGTLSPQDLKALCDYLKADYDPDRDFLPPEFPRRLAKR

SEQ ID NO: 443 - protein, 468 aa, Pseudomonas sp.
MNPFSPLVLVILTCACACGETVFAREQAKQHADLLSVYQAASLNDAQLSAARHAYNAQREAVPQARAGLLPNLTAGATSEVTRLERDEPSLSRERSGTTFRANLSQPLFRIDRWFQLKAAQSSVDQAELELSAKEQNLLLTAAQAYFETLRQLDAFAAAQAEEAALLRQRDQAQGRLDDGASSITDVYDAQAAYDNARANRQLVQRKVDDAYEALERLTKHSYTSIAGMRHQMPIEAPIPNDARAWVDIAVRQNLELQASERAVSAAEQTLSQRKAGHAPTIDAVASYRKGDNDSFGYSTPTDFGANGYRDNVAQSSIGIELNIPLYSGGMTSSQIKESTERLYQSQDEQEDRRREVVLNTRNAFRGINAGIEQIAARRQSIVSGQKSVEANQVGVDVGSRNIADVLNAQRQLYAAVRDYNDARYDYILDNLKLKQAAGTLSPDDLRALAAYLKQDYDPARDFLPPGV

SEQ ID NO: 444 - protein, 477 aa, Pseudomonas sp.
MLRKLSLAVAVSCASTTTVWAAEAPLSTKTDLVSVYQEAVDNNADLAAARAQYGAQKEVVPQARAGLLPNLSGGAEVANVRTSIDQPSAIANRSAHSYQATLAQPLFRADRWFQYQAAKDVNEQAALQLSATEQNLILQTAESYFNVLRSQDNLASTKAEEAAFKRQLDQSNERFDVGLSDKTDVLQSQASYDTARANRIVAQRQVDDAFEALITLTNRQYNSIQGIVHTLPILPPAPNDAKAWVDTAAKQNLNLLASNYAVSSAEQTLKQRKAGHLPTLDAVAKYEKGDNDALGFSNPNAFGTPYSGNVEQSTVGLQLNIPIYSGGLTSSQVRESYSRLDQTEQQREGLRRQVVENTRNLHRAVNTDVEQVQARRQSIISNQSAVEATEIGYQVGTRNIVDVLDAQRQLYTSVRNYNNTRYDYILDNLRLKQAAGTLNPGDLQDLTRYLKADYNPDKDFLPPDLAKAAEAQLKARP

SEQ ID NO: 445 - protein, 477 aa, Pseudomonas sp.
MLRKLSLAIAVSCASNGVVWAADVPSTVKTDLVSVYQEAVDNNADLAAARADYGARREVVPQARAGLLPNLSAGAEMMNTRTKLDEPSMTSNRSGNAWSATLAQPIFRADRWFQLQAAEAVNEQAALELSATEQNLILQTAQNYFAVLRAQDNLAATKAEEAAFKRQLDQSNERFDVGLSDKTDVLQSQASYDTARANRIIAERQVQDAFEALVTLTNREYTAIQGVVHSLPVQVPTPNDAKAWVETAGRQNLNLLATNYAVSAAEETLRQRKAGHAPTLDAVAKYQKGDNDSLGFTNPSQLGVRYSGDVEQTSVGLQLNIPIYSGGLTSSQVREAYQRLSQSEQQRESLRRQVVENTRNLHRAVNTDVEQVQARKQSIISNQSALEATEIGYQVGTRNIVDVLDAQRQLYTSVRDYNNSRYDYILDNLSLKQAAGTLSPQDLQDLKRYLKPDYNPDKDFLPPDLAAAAAKNFERRP

SEQ ID NO: 446 - protein, 479 aa, Pseudomonas sp.
MLRKLSLAVAVSCATNGLVWAAEAPLSANTDLVSVYQEAAHNNADLAAARAQYGAQKEVVPQARAGLLPNLSAGADSNNVRTQIDQPAATANRDAHSWRATLSQPLFRADRWFQLQAAQAVNEQASLQLSASEQNLILQSAESYFAVLRAQDNLASTKAEENAFKRQLDQSNERFDVGLSDKTDVLQSQASYDTARANRILAQRQVDDAFEALITLTNRQYNSIQGIVHTLPVLPPAPNDAKAWVETAGRQNLNLLASNYAVTAAEETLRQRKAGHLPTLDAVAQYEKGDNDALGFSNPNQLPIPYGGDVSQRTVGLRLNIPIYSGGLTSSQVRESYSRLDQTEQQREGLRRQVVENTRNLHRAVNTDVEQVQARRQSIISNQSAVEATEIGYQVGTRNIVDVLDSQRQLYASVRNYNNSRYDYILDNLRLKQAAGTLNPGDLQDLARYLKADYNPDKDFLPPDLAKAAAEQLKARPGY

SEQ ID NO: 447 - protein, 476 aa, Pseudomonas sp.
MLRKLSLAVAVSCASNGMAWAAEAPLTTRTDLVSVYQEALDNNADLAAARAQYGAQKEVVPQARAGLLPNLSAGADINNTRTKFDEPSMASTRSGNVYQATLAQPLFRADRWFQLQAAKDINEQASLQLSATEQNLILQSAENYFAVLRAQDNLASTKAEEAAFKRQLDQSNERFDVGLSDKTDVLQAQASYDTARANRILAQRQVDDAFEALITLTNREYNSIQGIVHTLPVLAPTPNDAKAWVDTAARQNLNLLASNYAVSAAEETLKQRKAGHAPTLDAVAQYKKGDNDALGFSNPNPLTRYGSDVEQRSIGLQLNIPIYSGGLTSSQVRESYSRLSQTEQQRESLRRQVVENTRNLHRAVNTDVEQVQARRQSIISNQSAVEATEIGYQVGTRNIVDVLDAQRQLYTSVRNYNNSRYDYILDNLRLKQAAGTLNPGDLQDLSRYLKADYNPDRDFLPPDLAKAAQEQLKARP

SEQ ID NO: 448 - protein, 482 aa, Pseudomonas sp.
MLRRLSLAAAVAAATGVAWAAQPTPLPTKTDLISVYKEAVDNNADLAAAQADYLARKEVVPQARAGLLPQLGAGGRIGDSRIALDEPAATVKRSSHVVQASLSQPLFRADRWFQWQAAKETSDQAKLEFSATQQDLILRSAETYFTVLRAQDNLATSKAEEAAFKRQLDQANERFDVGLSDKTDVLEAQASYDTARANRLIAEQRVDDAFQALVTLTNRDYSAIEGMRHTLPVVPPAPNDAKAWVDTAVQQNLRLLASNYAVNAAEETLRQRKAGHLPTLDAVAQYQKGDNDALGFANSAANPLVHYGKDVEERTIGLELNIPIYSGGLTSSQVRESYQRLNQSEQSREGQRRQVVQDTRNLHRAVNTDVEQVQARRQAIISNQSSLEATEIGYQVGTRNIVDVLNAQRQLYAAVRDYNNSRYDYILDTLRLKQAAGTLSPADLEALSAYLKQDYDPDKDFLPPDLAKAASEQLQSKPRQQY

SEQ ID NO: 449 - protein, 480 aa, Pseudomonas sp.
MLRRLSLAIAVAAASVGLVQAAEAPLSSKTDLVTVYQEAAKNNADIAAARADYQARREVVPQARAGLLPNLSAGANYGDTRTEIDSPSATVSRSGLVYQANLSQPLFRADRWFQLQAAEATSEQAALELSATEQNLILQSAETYFAVLRAQDNLASTRAEEAAFKRQLDQANERFDVGLSDKTDVLEAQAGFDTARANRLLAERAVDDAFEALVTLTNRDYVAVEGIVHSLPVLAPTPNDAKSWVDTAAAQNLNLQASLYAVNAAEENLRQRKAGHAPTLDAVASYQKGDNDSLGFTNSGSPLSPPYSGDVSQRTIGLQLNIPLYSGGLTSSQVREAYQRLGQTEQLRESLRRQVVQNTRNLFRAVNTDVETVQARRQSIISNQSALEATEIGYQVGTRNIVDVLDAQRQLYSAVRNYNDARYDYILNNLRLKQAAGTLSPADLEALGSFLKPDYNPDKDFLPPDLASAAEERLQNNQDF

SEQ ID NO: 450 - protein, 482 aa, Pseudomonas sp.
MLRRLSLAAAVAAATGVAWAAQPTPLPTKTDLISVYKEAVDNNADLAAAQADYLARKEVVPQARAGLLPQLGAGARVGDTRIAFDERPATVKRNSQVVQATLSQPLFRADRWFQWQAAKETSDQARLEFSATQQDLILRSAETYFTVLRAQDNLATSKAEEAAFKRQLDQANERFDVGLSDKTDVLEAQASYDTARANRLIAEQRVDDAFQALVTLTNRDYSAIEGMRHTLPVVPPAPNDAKAWVDTAVQQNLRLLASNYAVNAAEETLRQRKAGHLPTLDAVAQYQKGDNDALGFANSAANPLVHYGKYVDERSIGLELNIPIYSGGLTSSQVRESYQRLNQSEQSREGQRRQVVQDTRNLHRAVNTDVEQVQARRQAIISNQSSLEATEIGYQVGTRNIVDVLNAQRQLYAAVRDYNNSRYDYILDTLRLKQAAGTLSPADLEALSAYLKQDYDPDKDFLPPDLAKAAAEQLQSRPRQQY

SEQ ID NO: 451 - protein, 477 aa, Pseudomonas sp.
MLRKLSLAIAVSCASNGVAWAVDTPTTVKTDLVSVYQEAVDNNADLAAARADYGARREVVPQARAGLLPNLSAGAEMMNTRTKLDEPSITSNRSGNAWSATLAQPIFRADRWFQLQAAEAVNEQAALELSATEQNLILQSAENYFAVLRAQDNLASTKAEEAAFKRQLDQSNERFDVGLSDKTDVLQSQASYDTARANRIIAERQVQDAFEALITLTNRQYSAIQGVVHTLPVQVPTPNDAKAWVETAGRQNLNLQATNHAVAAAEETVRQRKAGHAPTLDAVAKYQKGDNDNLGFTNPSQNGVRYGGDVEQTSVGLQLNIPIYSGGLTSSQVREAYARLTQSEQQRESLRRQVVENTRNLHRAVNTDVEQVQARKQSIISNQSALEATEIGYQVGTRNIVDVLDAQRQLYTSVRDYNNSRYDYILDNLRLKQAAGTLSPQDLQDLGRYLKADYNPDKDFLPPDLAAAAAKNFERRP

SEQ ID NO: 452 - protein, 477 aa, Pseudomonas sp.
MLRKLSLAIAVSCASHGVAWAVDTPTTVKTDLVSVYQEAVDNNADLAAARADYGARREVVPQARAGLLPNLSAGAEMMNTRTKLDEPSVTSNRSGNAWSATLAQPIFRADRWFQLQAAESVNEQAALELSATEQNLILQSAENYFAVLRAQDNLASTKAEEAAFKRQLDQSNERFDVGLSDKTDVLQSQASYDTARANRIIAERQVQDAFEALITLTNRQYTAIQGVVHTLPVQVPTPNDAKAWVETAGRQNLNLLATNHAVAAAEETVRQRKAGHAPTLDAVAKYQKGDNDNLGFTNPSQNGVRYGGDVEQTSVGLQLNIPIYSGGLTSSQVREAYARLTQSEQQRESLRRQVVENTRNLHRAVNTDVEQVQARKQSIISNQSALEATEIGYQVGTRNIVDVLDAQRQLYTSVRDYNNSRYDYILDNLRLKQAAGTLSPQDLQDLGRYLKADYNPDKDFLPPDLAAAAAKNFERRP

SEQ ID NO: 453 - protein, 479 aa, Pseudomonas sp.
MLRKLSLVVAVSFASSGLTWAADMPLPTQTGLLNVYQQAVDNNADLAASRADYDARKEAVPQARAGLLPNISGSAQSNNTRTSIDQPRTVATRSGNVYQATLSQPIFRADRWFQLQAAEASNEQAALELSATEQNLILQAAQSYFSVLRAQDNLASTKAEEAAFKRQLDQANERFDVGLSDKTDVLQAQASYDTSRASRILAKRQVDDAFQALVTLTNREYNSIEGIVHTLPVLAPTPNDAKAWVDTAAQQNLNLLASNYAVSAAEETLRQRKAGHAPTLDAVATYQRGDNDALGFSNPNYTGRNYGGDVEQRSIGVQLNIPIYSGGLTSSQVREAYARLSQSEQRRESLRRQVVENTRNLHRAVNTDVEQVQARKQSIISNQSALEATEIGYQVGTRNIVDVLDAQRQLYASVRDYNNTRYDYILDNLRLKQAAGTLNPGDLQDLSRYLKADYNPDKDFLPPDLATAAQKNFERPAKR

SEQ ID NO: 454 - protein, 484 aa, Pseudomonas sp.
MLRRLPLALAVATLTAGGAWAQETVPLASKTDLVSVYQQAVNNNADLAAARAQFRARQEVVPQARAGLLPNLSAGAELSDTETDVDTSMGSTSLSRSGTAYQATLSQPIFRADRWFQLKAAEAVSEQAAIEYSIAEQDLILQSAQAYFAVLRAQDNLAAVKAEEAAFKRQLDQANERFEVGLSDKTDVLEAQAGFDTARANRSIAQRQVEDAFQALFTLTNREYVALEGIRHTLPVLAPVPNEAKAWVDTATRQNLNLLAVNYAVTAAEETLRQRRSGHAPTLDAVASYRKGDNDALGFSNTQSIPGFDMPRYTGDVEQRSIGLQLNIPLYSGGLTTSQSREAYYQLSQSEQQRESLRRQVVEATRNLHRAVNTDIEQVQARRQSIISNQSALEATEIGYQVGTRNIVDVLDTQRRLYAAVRDYNNARYDYILDNLRLKQAAGTLNPDDLQQLAAYLKPDYNPDTDFLPPDLPQTIGQPVRVDY

SEQ ID NO: 455 - protein, 479 aa, Pseudomonas sp.
MLRKLSLAVAVSCATNGMVWAAEAPLSTNTDLVSVYQEAAHNNADLAAARAQYGAQKEVVPQARAGLLPNLSAGADSNNVRTQIDQPAATANRDAHSWRATLSQPLFRADRWFQLQAAEAVNEQASLQLSATEQNLILQSAESYFAVLRAQDNLASTKAEENAFKRQLDQSNERFDVGLSDKTDVLQSQASYDTARANRIVAQRQVDDAFQALITLTNRQYNSIQGIVHTLPVLPPAPNDAKAWVETAGRQNLNLLASNYAVTAAEETLRQRKAGHLPTLDAVAQYEKGDNDALGFSNPNQLPIPYGGDVSQRTIGLRLNIPIYSGGLTSSQVRESYSRLDQTEQQREGLRRQVVENTRNLHRAVNTDVEQVQARRQSIISNQSAVEATEIGYQVGTRNIVDVLDAQRQLYSSVRNYNNSRYDYILDNLRLKQAAGTLNPGDLQDLARYLKADYNPDKDFLPPDLAKAAAEQLKARPGY

SEQ ID NO: 456 - protein, 478 aa, Pseudomonas sp.
MLRKLSLAIAVSCATNVMAMETQAPLASQTGLVSVYHEAVDNNADLAAARADYAARKEVVPQARAGLLPNLSGGAESSSVRTSIDQPSLTTTRSAVVYQATLAQPIFRIDRWFQLKAAESVNEQAALQLSATEQNLILQTAESYFSVLRAQDTLAATKAEEAAFKRQLDQSRERFDVGLSDKTDVLQSQASYDTSRANRIVAQRQVDDAFEALITLTNRQYNSIEGMRHTLPILPPSPNNAKIWVETAAQQNLNLLASNFAVDAAQETLKQRKAGHAPTLDAVAQYKTGDNDGFGLSNPNALQQRYSGDVSQRSIGIQLNIPIYSGGLTSSQVRESYSRLDQTEQQREGLRRQVVENTRNLHRAVNTDVEQVQARKQSIISNQSALEATEIGYQVGTRNIVDVLDAQRQLYNSVRDYNNTRYDYILDNLRLKQVAGTLNPGDLDALSRYLKADYNPDKDFLPPDLAQAAEANMRSPGN

SEQ ID NO: 457 - protein, 479 aa, Pseudomonas sp.
MLRKLSLVVAVSFASSGLTWAAELPMPTQTGLLSVYQQAVDNNADLAASRADYDARKEAVPQARAGLLPNISGSAQNSSTRTSIDQPRAVASRSGNLYQATLSQPIFRADRWFQLQAAEASNEQAALELSATEQNLILQSAQSYFSVLRAQDNLASTKAEEAAFKRQLDQANERFDVGLSDKTDVLQAQASYDTSRASRLLAQRQVEDAFQALVTLTNREYNSIEGIVHTLPVLAPTPNDAKAWVDTAAQQNLNLLASNYAVNAAEETLRQRKAGHAPTLDAVATYQRGDNDALGFSNPNYTGRNYGGDVEQRSIGVQLNIPIYSGGLTSSQVREAYARLSQSEQRRESLRRQVVENTRNLHRAVNTDVEQVQARKQSIISNQSALEATEIGYQVGTRNIVDVLDAQRQLYASVRDYNNTRYDYILDNLRLKQAAGTLNPGDLQDLSRYLKADYNPDKDFLPPDLATAAQKNFERPAKR

SEQ ID NO: 458 - protein, 479 aa, Pseudomonas sp.
MLRKLSLALAVSCASNGMAWAAEAPLTTKTDLVSVYQEAVDNNADLAAARAQYGAQKEVVPQARAGLLPNLSAGADLNNTRTELDQPAMTANRSSTVYQATLSQPIFRADRWFQLQAAKSVDEQASLQLSATEQNMILQSAESYFNVLRSQDNLASTKAEEAAFKRQLDQSNERFDVGLSDKTDVLQSQASYDTARANRILAQRQVDDAFEALITLTNRQYNSLQGIVHTLPILPPAPNDAKAWVDTAAKQNLNLLASNYAVTAAEDTLRQRKAGHAPTLDAIAQYKKGDNDGLGFTNPSLTGQRYGGDAEQRTIGLQLSIPLYSGGLTSSQVRESYSQLTQTEQQREALRRQVVENTRNLHRAVNTDVEQVQARRQSIISNQSAVEATEIGYQVGTRNIVDVLDAQRQLYTSVRNYNNARYDYILDNLRLKQAAGTLSPADLQDLSRYLKPDYNPDQDFLPPDLAQAAAAQLRSRPAQ

SEQ ID NO: 459 - protein, 477 aa, Pseudomonas sp.
MLRKLSLALAVSCASQTMAWAADVPATVKTDLVSVYQEAVQNNADLAAARADYGARREIVPQARAGLLPNLSAGAEMQSTRTKLDEPSITSNRSGNAWSATLAQPIFRADRWFQLQAAEAVNEQAALELSATEQNLILQTAQSYFAVLRAQDNLAASKAEEAALKRQLDQSNERFDVGLSDKTDVLQSQASYDTARANRIIADRQVQDAFEALVTLTNREYTAIQGVVHSLPVQVPVPNDAKAWVETAGRQNLNLLATNYAVSAAEETLRQRKAGHAPTVDAVAKYQKGDNDNLGFSNPSPLGVRYGGDVEQTTVGLQLNIPIYSGGLTSSQVREAYQRLSQSEQQRESLRRQVVENTRNLHRAVNTDVEQVQARKQSIISNQSALEATEIGYQVGTRNIVDVLDAQRQLYTSVRDYNNSRYAYILDNLSLKQAAGTLSPQDLQDLKRYLKPDYNPDKDFLPPDLAAAAAKNFERRP

SEQ ID NO: 460 - protein, 477 aa, Pseudomonas sp.
MLRKLSLALAVSCASNGMAWAAEAPLSTKTDLVSVYQEAVDNNADIAAARAQYGAQKEVVPQARAGLLPNLSAGANMNDTRTALDQPSVTSNRSGTVYQATLSQPIFRADRWFQFQAAKDVNEQAALQLSATEQNLILQSAESYFNVLRSQDNLASTKAEEAAFKRQLDQSNERFDVGLSDKTDVLQSQASYDTARANRIVAQRQVDDAFEALITLTNRQYNSIQGIVHTLPILPPAPNDAKAWVDTAAKQNLNLLASNYAVSAAEETLKQRKAGHAPTLDAVAQYRKGDNDALGFTNPSATGVHYGGDVEQRTVGLQLSIPIYSGGLTSSQVRESYSRLDQTEQQREALRRQVVENTRNLHRAVNTDVEQVQARRQSIISNQSAVEATEIGYQVGTRNIVDVLDAQRQLYTSVRNYNNTRYDYILDNLRLKQAAGTLNPGDLQDLTRYLKADYNPDKDFLPPDLAKAAAEQLKARP

SEQ ID NO: 461 - protein, 483 aa, Pseudomonas sp.
MLRRLTLALAVASASVGSAWAEQAHVPLTTKTDLVSVYNEAANNNADLAAARADYQARREVVPQARAGLLPNLTGGATVGNTRTSLDQPSTTLSRSGTSYQANLSQPLFRADRWYQLQAAEATSEQAALDLSATEQNLILSSAETYFAVLRAQDTLASTKAEEAAFKRQLDQSNERFEVGLSDKTDVLEAQAGYDTARANRIIAQRQVDDAFQALMTLTNRDYTSIEGIKHTLPILAPTPNDAKAWVDTAAAQNLNLQASNFAVDAANETLRQRKAGHAPTLDAVASYEQGDNDSLGFTNTGIPTGAQPYGSDVSQTTLGLQLNIPIYSGGLTSSQVREAYQRLDQTEQLRESLRRKIVQDTRNFHRAVNTDVETVQARKQSIISNQSALEATQIGYQVGTRNIVDVLDAQRQLYSSVRNYNDARYDYILNNLRLKQAAGTLSPGDLEALGAYLKPDYNPDKDFLPPDLAKAAEAQMHGNPQY

SEQ ID NO: 462 - protein, 480 aa, Pseudomonas sp.
MLRRLSLAIAVAAASVGLVQAAEAPLSSKTDLVTVYQEAAQNNADIAAARADYQARREVVPQARAGLLPNLSAGANYGDTRTEIDSPSATISRSGLVYQANLSQPLFRADRWFQLQAAEATSEQAALELSATEQNLILQSAETYFAVLRAQDNLASTRAEEAAFKRQLDQANERFDVGLSDKTDVLEAQAGFDTARANRLLAERAVDDAFEALVTLTNRDYVAVEGIVHTLPVLAPTPNDAKSWVDTAAAQNLNLQASLYAVNAAEENLRQRKAGHAPTLDAVASYQQGDNDSLGFTNSGAFGAPRYSGDVSQRSIGLQLNIPLYSGGLTSSQVREAYQRLGQTEQLRESLRRQVVQNTRNLFRAVNTDVETVQARRQSIISNQSALEATEIGYQVGTRNIVDVLDAQRQLYSAVRNYNDARYDYILNNLRLKQAAGTLSPADLEALGSFLKPDYNPDKDFLPPDLASAAEERLKNNQDF

SEQ ID NO: 463 - protein, 482 aa, Pseudomonas aeruginosa
MLRRLSLAAAVAAATGVAWAAQPTPLPTKTDLISVYKEAVDNNADLAAAQADYLARKEVVPQARAGLLPQLGAGGRIGDSRIALDEPAATVKRSSHVVQASLSQPLFRADRWFQWQAARETSDQAKLEFSATQQDLILRSAETYFTVLRAQDNLATSKAEEAAFKRQLDQANERFDVGLSDKTDVLEAQASYDTARANRLIAEQRVDDAFQALVTLTNRDYSAIEGMRHTLPVVPPAPNDAKAWVDTAVQQNLRLLASNYAVNAAEETLRQRKAGHLPTLDAVAQYQKGDNDALGFANSAANPLVHYGKDVEERTIGLELNIPIYSGGLTSSQVRESYQRLNQSEQSREGQRRQVVQDTRNLHRAVNTDVEQVQARRQAIISNQSSLEATEIGYQVGTRNIVDVLNAQRQLYAAVRDYNNSRYDYILDTLRLKQAAGTLSPADLEALSAYLKQDYDPDKDFLPPDLAKAAAEQLQSKPRQQY

SEQ ID NO: 464 - protein, 482 aa, Pseudomonas aeruginosa
MLRRLSLAAAVAAATGVAWAAQPTPLPTKTDLISVYKEAVDNNADLAAAQADYLARKEVVPQARAGLLPQLGAGGRIGDSRIALDEPAATVKRSSHVVQASLSQPLFRADRWFQWQAAKETSDQAKLEFSATQQDLILRSAETYFTVLRAQDNLATSKAEEAAFKRQLDQANERFDVGLSDKTDVLEAQASYDTARANRLIAEQRVDDAFQALVTLTNRDYSAIEGMRHTLPVVPPAPNDAKAWVDTAVQQNLRLLASNYAVNAAEETLRQRKAGHLPTLDAVAQYQKGDNDALGFANSAANPLVHYGKDVEERTIGLELNIPIYTGGLTSSQVRESYQRLNQSEQSREGQRRQVVQDTRNLHRAVNTDVEQVQARRQAIISNQSSLEATEIGYQVGTRNIVDVLNAQRQLYAAVRDYNNSRYDYILDTLRLKQAAGTLSPADLEALSAYLKQDYDPDKDFLPPDLAKAAAEQLQSKPRQQY

SEQ ID NO: 465 - protein, 482 aa, Pseudomonas aeruginosa
MLRRLSLAAAVAAATGVAWAALPTPLPTKTDLISVYKEAVDNNADLAAAQADYLARKEVVPQARAGLLPQLGAGGRIGDSRIALDEPAATVKRSSHVVQASLSQPLFRADRWFQWQAAKETSDQAKLEFSATQQDLILRSAETYFTVLRAQDNLATSKAEEAAFKRQLDQANERFDVGLSDKTDVLEAQASYDTARANRLIAEQRVDDAFQALVTLTNRDYSAIEGMRHTLPVVPPAPNDAKAWVDTAVQQNLRLLASNYAVNAAEETLRQRKAGHLPTLDAVAQYQKGDNDALGFANSAANPLVHYGKDVEERTIGLELNIPIYSGGLTSSQVRESYQRLNQSEQSREGQRRQVVQDTRNLHRAVNTDVEQVQARRQAIISNQSSLEATEIGYQVGTRNIVDVLNAQRQLYAAVRDYNNSRYDYILDTLRLKQAAGTLSPADLEALSAYLKQDYDPDKDFLPPDLAKAASEQLQSKPRQQY

SEQ ID NO: 466 - protein, 464 aa, Pseudomonas brassicacearum
MSRAQRVLLFACLFPSFVFSAPTAERAELMAVYRQAVSHNSDLAAARADYAAQQELVPQARANLLPVISTGATFEATRLSRDEPALERGRSGNTIQANLKQPLVNAAFWYEFKAAKASTVQAALELSAKEQALILQTAQAYFETLRATDELAASEAEETALKQQMDQAQARLKGGLSSITDVLDAESAFDNAQANRQLAQRKVEDAFEQLVHLTNQQYTSIEGMQHQLPVLAPIPNDTKSWVEGAVEQNLMLLASNYAVEAAEERLRQRQSGHAPTVDAVASYRKGDNDSFGYSNPTDFGRDGYRGNVAQSSIAVEVTVPLYSGGRVSSQAHEAYQRLTQSEELRESQRREVVLDTRNYLRAVNSDIQQIKARRQTILSSQKSLKASKVGVDIGTRNTVDVLNAQRQLFKSVRDYNDARYDYILNNLRLKQAAGTLSADDLQQLAEYLKADYQPQRDFLPPELM

SEQ ID NO: 467 - protein, 479 aa, Pseudomonas coronafaciens
MLRKLSLVVAVSFASSGLTWAADLPMPTRTGLLNVYRQAVDNNADLAASRADYDARKEAVPQARAGLLPNISGSVQNTNTRTSIDRPSAVATRSGTVYQATLSQPIFRADRWFQLQAAEAVNEQAALELSATEQNLILQSAQSYFSVLRAQDNLASTKAEEAAFKRQLDQANERFDVGLSDKTDVLQAQASYDTSRASRLLAKRQVDDAFQALVTLTNREYNSIEGIVHTLPVLAPTPNDAKAWVDTAAQQNLNLLASNYAVSAAEETLRQRKAGHAPTLDAVATYQRGDNDSLGFNNPNYTGQNYGGDVEQRSIGVQLNIPIYSGGLTSSQVREAYSRLSQTEQRRESLRRQVVENTRNLHRAVNTDVEQVQARKQSIISNQSALEATEIGYQVGTRNIVDVLDAQRQLYASVRDYNNTRYDYILDNLRLKQAAGTLNPGDLQDLSRYLKPDYNPDKDFLPPDLATAAQKNFERPAQR

SEQ ID NO: 468 - protein, 491 aa, Pseudomonas coronafaciens
MLSQGRTVYVDAFKTTLLFLFSIVALPLLAAPAPADKSAVYGAGSQGRDMPRQRADLLGVYEAASHNDARLSAARHAYRAEIEAVPQARAGLLPSLTAGATTEVTGLKRDEPALSRERSGTTVRANLSQPLFRIDRWFQLKAAQSSVIQAELELAAQEQNLILTAAQTYFEILRQLDAFAAAQAEEAALLRQRDQAQGRLEDGASSITDVYDAQAAYDNARADRQQFQRKVDDAYEALERLTRRTYSAIAGMGHQLPVEAPVPNDARAWVDTAVRQNLELQASEYAVSAATHTLSQRKSGHAPTIDAVASYRKGDNDSFGYSNPTDFGSNGYRDRVAQSSVGIELNIPLYSGGMTNSQIKESTERLYQSQDEQEDRRREVVLNTRNAFRGINAGIEQIIARRQSIVSGQKSVEANQVGVDLGSRNIADVLNAQRQLYAAVRDYNDARYDYILDNLKLKQAAGTLSPDDLKALARYLKQDYDPARDFLPTGS

SEQ ID NO: 469 - protein, 477 aa, Pseudomonas donghuensis
MLRKLSLALAVSCATNGMAWAADVPLSAKTDLVSVYQEAVNNNADLAAARANYGAQKEVVPQARAGLLPNLSAGAEMMSNRTKIDSPSVTSNRSGNSWQATLAQPIFRADRWFQLQAAEAVNEQAALELSATEQNLILQSAENYFAVLRAQDTLASTKAEEAAFKRQLDQSNERFDVGLSDKTDVLQSQASYDTARANRIVAQRQVDDAFEALITLTNRDYNSVQGIVHTLPVQVPVPNDAKAWVETAGKQNLNLLATNYAVSAAEETLRQRKAGHAPTVDAVARYQKGDNDSLGFTNPNLTGQNYSGDVEQTSIGLQLNIPIYSGGLTSSQVREAYQRLSQSEQQRESLRRQVVENTRNLHRAVNTDVEQVQARKQSIISNQSALEATEIGYQVGTRNIVDVLDAQRQLYTSVRDYNNTRYDYILDNLRLKQAAGTLSPQDLTDLSRYLKSDYNPDKDFLPPDLAAAAKANFEALP

SEQ ID NO: 470 - protein, 477 aa, Pseudomonas putida
MLRKLSLAIAVSCASNGVVWAADVPSTVKTDLVSVYQEAVDNNADLAAARADYGARREVVPQARAGLLPNLSAGAEMMNTRTKLDEPSITSNRSGNSWSATLAQPIFRADRWFQLQAAEAVNEQAALELSATEQNLILQTAQNYFAVLRAQDNLAATKAEEAAFKRQLDQSNERFDVGLSDKTDVLQSQASYDTARANRIIAERQVQDAFEALVTLTNREYTAIQGVVHSLPVQVPTPNDAKAWVETAGRQNLNLLATNYAVSAAEETLRQRKAGHAPTLDAVAKYQKGDNDSLGFTNPSQLGVRYSGDVEQTSVGLQLNIPIYSGGLTSSQVREAYQRLSQSEQQRESLRRQVVENTRNLHRAVNTDVEQVQARKQSIISNQSALEATEIGYQVGTRNIVDVLDAQRQLYTSVRDYNNSRYDYILDNLSLKQAAGTLSPQDLQDLKRYLKPDYNPDKDFLPPDLAAAAAKNFERRP

SEQ ID NO: 471 - protein, 478 aa, Pseudomonas putida
MLRKLSLAIAVSCASNGVAWAVDVPSTVKTDLVSVYQEAVDNNADLAAARADYGARREVVPQARAGLLPNLSADAEMLNTRTKLDEPSITSNRSGNSWSATLAQPIFRADRWFQLQAAEAVNEQAALELSATEQNLILQTAQNYFAVLRAQDNLAATKAEEAAFKRQLDQSNERFDVGLSDKTDVLQSQASYDTARANRIIAERQVQDAFEALVTLTNREYTSIQGVVHTLPVQVPTPNDAKAWVETAGRQNLNLLATNYAVSAAEETLRQRKAGHAPTLDAVAKYQKGDNDNLGFTNPAPQQNVRYGGDVEQTSVGLQLNIPIYSGGLTSSQVREAYQRLSQSEQQRESLRRQVVENTRNLHRAVNTDVEQVQARKQSIISNQSALEATEIGYQVGTRNIVDVLDAQRQLYTSVRDYNNSRYDYILDNLSLKQAAGTLSPQDLQDLKRYLKPDYNPDKDFLPPDLAAAAAKNFERRP

SEQ ID NO: 472 - protein, 478 aa, Pseudomonas putida
MLRKLSLAIAVSCASNGVVWAADMPTTVKTDLVSVYQEAVDNNADLAAARADYGAQKEVVPQARAGLLPNLSAGAEMQNTRTKLDQPSATINRSGNSWSATLSQPIFRADRWFQLQAAEAVNEQAALQLSATEQNLILQTAQDYFTVLRAQDNLAATKAEEAAFKRQLDQSNERFDVGLSDKTDVLQSQASYDTARANRIVAERQVQDAFEALVTLTNREYSSIQGVVHTLPVKVPTPNDAKAWVETAGRQNLNLLATNHAVAAAEETLRQRKAGHAPTLDAVAQYQKGDNDNLGFSNSAALPNVHYSGDVEQTTVGLQLNIPIYSGGLTSSQVREAYQRLSQSEQQRESLRRQVVENTRNLHRAVNTDVEQVQARKQSIISNQSALEATEIGYQVGTRNIVDVLDAQRQLYTSVRDYNNSRYDYIIDNLSLKQAAGTLSPQDLQDLKQYLKPDYNPDKDFLPPDLAAAAAKNFERRP

SEQ ID NO: 473 - protein, 455 aa, Pseudomonas putida
MLRKTLLPLLFLAPLAQAVAAERATLMSVYAQVVEHNSDMAAARENYLARREAVPLARARLLPQVALDAEAGDVRSDRHRVEQRSGSLYRLSLDQPLFDLSRWFDFKAAQSENEQAELDFSAFQQQLILDTASRYFDTLLAEDNLATAKAELRAFDRQLQQTRLSYDAGLSDQNDMLSAQASFDRASANLIDSQRRSEDAYQALMRLTGQAQPALAGIRHSLPVQAPVPARAEAWVEQAMAQNLRLRASQAAVSQAGQTLRSSKADHLPTFNLSMGYAEGDSDLMDSSAHFGQRSGNQRDSSVMVQMKLPLFSGGGTSARVRQATHELARTEYGRTSLEREVLEGSRNAFRAVVSDVEQVRARRQSIISSQGSLRATEAGYEVGSRNIVDILDAQRDLYNAVRDYNVSRYAYIVDSLRLKQQAGSLSPDDLRELAGYLKLDYEPERDFLPSDLRG

SEQ ID NO: 474 - protein, 471 aa, Pseudomonas putida
MRRRTPLIHTTFLASLCMCTTLLAEPQRAELLQVYQHAVEQDARLSAARHEFQALSERVPQARAGLLPTLNAGATFESTRMQRDEPALTRTRSGMVYQANLNQPLLRLDRWYALKAAEAGTAQAGLDLAAKEQSLILQTAEAYFDTLRALDALAAAKAEAKALKRQQEQAQGRLANGASSITDVLDAQAAYDNASANRQLAQRKVDDAFEVLYRLTRQDYAQIAGIAHQLPVQPPVPNDASTWVDQAVQSNLSLLSSGHAVDAAVQASRQRKAGHAPTLDAVASYRKGDNDRFGYSNPTDFGRNGYRDDIAQGTLGLELNIPLYAGGLTSSQVREAGERLAQSEDEHEDRRREVVLQTRNLHRAVNADVEQVQARQQSIRSSQASLKANQVGRALGSRNTADVLNAERQLYRAVREYNDARYDYIIDSLKLKQAAGSLAPQDLLELSHYLSRDYDPDRDFLPPEARRPVAG

SEQ ID NO: 475 - protein, 455 aa, Pseudomonas soli
MLRKTLLPLLFLAPLAQAVAAERATLMSVYAQVVEHNSDMAAARENYLARREAVPLARARLLPQVALDAEAGDVRSDRRQVEQRSGSLYRLSLDQPLFDLSRWFDFKAAQSENEQAELDFSAFQQQLILDTASRYFDTLLAEDNLATAKAELRAFDRQLQQTRLSYDAGLSDQNDMLSAQASFDRASANLIDSQRRSEDAYQALMRLTGQAQPALAGIRHSLPVQAPVPARAEAWVEQAMAQNLRLRASQAAVSQAGQTLRSSKADHLPTFNLAMGYAEGDSDLMDSSAHFGQRSGNQRDSSVMVQMKLPLFSGGGTSARVRQATHELARTEYGRTSLEREVLEGSRNAFRAVVSDVEQVRARRQSIISSQGSLRATEAGYEVGSRNIVDILDAQRDLYNAVRDYNVSRYAYIVDSLRLKQQAGSLSPEDLRELAGYLKLDYEPERDFLPSDLRG

SEQ ID NO: 476 - protein, 480 aa, Pseudomonas sp.
MLRRLSLALAVASAASGMAWAADAPAGSKTGLVTVYNEAVKNNADLAAARANYGAISEVVPQARAGLLPNLSAGADMTNTDTQVDRSIGDLDSSRSGTSYRASLSQPLFRADRWFQYQAAKATNEQASLALSATEQNLILQSAETYFAVLRAQDTLASTKAEEAAFKRQLDQANERFDVGLSDKTDVLEAQAGYDTSRANRIIAERNVQDAFQALVTLTNNEYAAIEGIQHTLPILVPVPNDAKAWVDTAAQQNLNLQSSNFAVDAAEETLRQRKSGHAPTLDAVAQYQKGDNDSLGFNNSDALSVYDGDAEQTTIGLQLNIPIYSGGLTSSQVREAYQTLTQTEQERESLRRQIVENTRNLHRAVNTDVETVQARKQSIISNQSALEATEIGYQVGTRNIVDVLDAQRQLYSSVRNYNDARYDYIINNLRLKQAAGTLAPSDLDALSSFLKPDYNPDQDFLPPDLAKAAEAQLNGNPEY

SEQ ID NO: 477 - protein, 479 aa, Pseudomonas sp.
MLRRLSLAVAVASAFAGSAWAAEAPLSAKTDLVTVYKDAVDNNADLAAARANFEAVREAVPQARSGLLPQINGGATANDTRTKLDEPSATSSRSGIAYQASLSQPIFRADRWFQLQAAEASTEQASLEFSATQQNLILQTAEGYFAVLRAQDNLASTKAEEAAFKRQLDQANERFDVGLSDKTDVLEAQAGFDTARANRIIAERNVDDAFQALVTLTNREYASIEGIQHTLPVVVPAPNDAKAWVDTAAQQNLNLQASNYAVDAAEETLRQRKAGHAPTLDAVAQYQKGDNDSLGFTNSAGSPVRYGSDVEQRTIGLQLNIPIYSGGLTSSQVRESYQRLNQTEQQRESLRRQVVQNTRNYHRAVNTDVEQVKARKQSIISNQSALEATEIGYQVGTRNIVDVLDAQRQLYSSVRDYNNARYDYILDNLRLKQAAGTLSPSDLEALASFLKSDYDPDKDFLPPDLAKAAEASLKNKSNY

SEQ ID NO: 478 - protein, 479 aa, Pseudomonas syringae
MLRKLSLVVAVSFASSGLTWAAELPMPTQTGLLSVYQQAVDNNADLAASRADYDARKEAVPQARAGLLPNISGSAQNSSTRTSIDQPRAVATRSGNLYQATLSQPIFRADRWFQLQAAEASNEQAAMELSATEQNLILQSAQSYFSVLRAQDNLASTKAEEAAFKRQLDQANERFDVGLSDKTDVLQAQASYDTSRASRLIAQRQVDDAFQALVTLTNREYNSIEGIVHTLPVLAPTPNDAKAWVDTAAQQNLNLLASNYAVNAAEETLRQRKAGHAPTLDAVATYQRGDNDALGFSNPNYTGRNYGGDVEQRSIGVQLNIPIYSGGLTSSQVREAYARLSQSEQRRESLRRQVVENTRNLHRAVNTDVEQVQARKQSIISNQSALEATEIGYQVGTRNIVDVLDAQRQLYASVRDYNNTRYDYILDNLRLKQAAGTLNPGDLQDLSRYLKADYNPDKDFLPPDLATAAQKNFERPAKR

SEQ ID NO: 479 - protein, 479 aa, Pseudomonas syringae
MLRKLSLVVAVSLASSGLTWAADLPLPTKTGLLNVYQQAVDNNADLAASRADYDARKEAVPQARAGLLPNISGSVQNTNTRTSIDRPSAVATRSGTVYQATLSQPIFRADRWFQLQAAEAVNEQAALELSATEQNLILQSAQSYFSVLRAQDNLASTKAEEAAFKRQLDQANERFDVGLSDKTDVLQAQASYDTSRASRLIAKRQVDDAFQALVTLTNREYNSIEGIVHTLPVLAPTPNDAKAWVDTAAQQNLNLLASNYAVSAAEETLRQRKAGHAPTLDAVATYQRGDNDALGFNNPNYTGQNYGGDVEQRSIGVQLNIPIYSGGLTSSQVREAYSRLSQSEQRRESLRRQVVENTRNLHRAVNTDVEQVQARKQSIISNQSALEATEIGYQVGTRNIVDVLDAQRQLYASVRDYNNTRYDYILDNLRLKQAAGTLNPGDLQDLSRYLKPDYNPDKDFLPPDLATAAQKNFERPAQR

SEQ ID NO: 480 - protein, 479 aa, Pseudomonas syringae
MLRKLSLVVAVSFASSGLTWAADMPLPTQTGLLNVYQQAVDNNADLAASRADYDARKEAVPQARAGLLPNISGSAQSNNTRTSIDQPRAVATRSGNVYQATLSQPIFRADRWFQLQAAEASNEQAALELSATEQNLILQAAQSYFSVLRAQDNLASTKAEEAAFKRQLDQANERFDVGLSDKTDVLQAQASYDTSRASRILAKRQVDDAFQALVTLTNREYNSIEGIVHTLPVLAPTPNDAKAWVDTAAQQNLNLLASNYAVSAAEETLRQRKAGHAPTLDAVATYQRGDNDALGFSNPNYTGRNYGGDVEQRSIGVQLNIPIYSGGLTSSQVREAYARLSQSEQRRESLRRQVVENTRNLHRAVNTDVEQVQARKQSIISNQSALEATEIGYQVGTRNIVDVLDAQRQLYASVRDYNNTRYDYILDNLRLKQAAGTLSPGDLQDLSRYLKADYNPDKDFLPPDLATAAQKNFERPAKR

SEQ ID NO: 481 - protein, 468 aa, Pseudomonas syringae
MKHNFYLCALSLYLFALGAQEALAREPYSKRADLLAVYQAASLNDARLSAARHTYQAQLEATPQARSGLLPSLTAGATTEVTQLDRDEPALSRERSSTTFRANLSQPLFRIDRWYQLKAAEFTVSQAALELSAKEQELILTAAQAYFETLRQLDARAAAEAEEAALLRQRDQAQGRLEDGASSITDVYDAQAAYDNARANRQLIQRKVDDAYEALYRLTKNSYDSLDGMGHQLPVIPPNPNNPVLWVDAAIKQNLQLQAAEHAVSAAEQTLRQRKAGHAPTVDAVVSYRTGNNDGFGYSNPAESGTGGYRGNVAQSSIGVELSIPLYSGGMVNSQVRESTERLYQSQDEQEDRRREVVLNTRNAFRGINSDIEQIAARRQSIRSGRKSVEANKVGVDIGSRNVVDVLNAERQLYAAVRDYNDARYDYILDNLKLKQAAGSLSPDDLRSLSAYLNHDYDPSRDFLPPDI

SEQ ID NO: 482 - protein, 479 aa, Pseudomonas syringae
MLRKLSWVVAVSFASSGLTWAADLPMPIKTGLLNVYQQAVDNNADLAASRADYDARKEVVPQARAGLLPNISGSVQNTDTRTSIDQPRAVASRSGTVYQATLSQPIFRADRWFQLQAAEAVNEQAALELSATEQNLILQSAQSYFSVLRAQDNLASTKAEEAAFKRQLDQARERFDVGLSDKTDVLQAQASYDTSRASRLLAQRQVDDAFQALVTLTNREYNSIEGIVHTLPVLAPTPNDAKAWVDTAAQQNLNLLASNYAVSAAEETLRQRKAGHAPTLDAVATYQRGDNDALGFNNPNYTGRNYSGDVEQRSIGVQLNIPIYSGGLTSSQVREAYSRLSQSEQRRESLRRQVVENTRNLHRAVNTDVEQVQARKQSIISNQSALEATEIGYQVGTRNIVDVLDAQRQLYASVRDYNNTRYDYILDNLRLKQAAGTLNPGDLQDLSRYLKPDYNPDKDFLPPDLATAAQKNFERPAQR

SEQ ID NO: 483 - protein, 479 aa, Pseudomonas syringae
MLRKLSLVVAVSFASSGLTWAADMPLPTQTGLLNVYQQAVDNNADLAASRADYDARKEAVPQARAGLLPNISGSAQSNNTRTSIDQPRAVATRSGNVYQATLSQPIFRADRWFQLQAAEASNEQAALELSASEQNLILQAAQSYFSVLRAQDNLASTKAEEAAFKRQLDQANERFDVGLSDKTDVLQAQASYDTSRASRILAKRQVDDAFQALVTLTNREYNSIEGIVHTLPVLAPTPNDAKAWVDTAAQQNLNLLASNYAVSAAEETLRQRKAGHAPTLDAVATYQRGDNDALGFSNPNYTGRNYGGDVEQRSIGVQLNIPIYSGGLTSSQVREAYARLSQSEQRRESLRRQVVENTRNLHRAVNTDVEQVQARKQSIISNQSALEATEIGYQVGTRNIVDVLDAQRQLYASVRDYNNTRYDYILDNLRLKQAAGTLSPGDLQDLSRYLKADYNPDKDFLPPDLATAAQKNFERPAKR

SEQ ID NO: 484 - protein, 468 aa, Pseudomonas syringae
MKPFSSLCLVMLACTFACSETVFAREQPEQHADLLGVYQAAALNDAQLSAARHAYNAQREAVPQARAGLLPNLTAGATSEVTSLKRDEPSLTRERSGITFRANVSQPLFRIDRWFQLKAAQSSVSQAELELSAKEQNLLLTAAQAYFETLRQLDAYAAAQAEEAALLRQRDQAQGRLEDGASSITDVYDAQAAYDNARANRQLVQRKVDDAYEALERLTKHTYTSIAGMRHQMPVEAPIPNDARAWVDVTVRQNLQLQASEHAVSAAEQTLSQRKAGHAPTIDAVASYRKGDNDSFGYSNPTDFGSNGYRDNVAQSSIGIELNIPLYSGGMTNSQIKESTERLYQSQDEQEDRRREVVLTTRNAFRGINAGIEQIAARRQSIVSGQKSVEANQVGVDVGSRNIADVLNAQRQLYAAVRDYNNARYDYILDNLKLKQAAGTLSPDDLKALASYLKQDYDPARDFLPPGV

SEQ ID NO: 485 - protein, 479 aa, Pseudomonas yamanorum
MLRKLSLALAVSCATNGLVWAAEAPLTTKTDLVSVYQEAVDNNADLAAARAQYGAQKEVVPQARAGLLPNLSAGADINNVRTQLDQPAATVNRDAHSWRATLSQPLFRADRWFQLQAAEAVNEQAALQLSATEQNLILQSAENYFAVLRAQDNLASTKAEEAAFKRQLDQSNERFDVGLSDKTDVLQSQASYDTARANRILAQRQVDDAFEALITLTNRQYNSIQGIVHTLPVLPPSPNDAKAWVETAGRQNLNLLASNYAVTAAEETLKQRKAGHAPTLDAVAQYEKGDNDALGFSNPSAFGQPYHGDVSQRTVGLQLRIPLYSGGLTSSQVRESYSRLGQTEQQREGLRRQVVENTRNLHRAVNTDVEQVQARRQSIISNQSAVEATEIGYQVGTRNIVDVLDAQRQLYTSVRNYNNSRYDYILDNLRLKQAAGTLNPGDLQDLARYLKADYNPDKDFLPPDLAKAAAEQLKANPGY

SEQ ID NO: 486 - protein, 439 aa, Psychromonas sp.
MKIKQLTLATFIALTSFNSVVSADDLLQIYETAQLKDPIIQKSKAQYDLYLERVSASNASLLPQIGFGLNAGKSKDKDDINSNTNYGANLSLSQSLYNGTYWKELDLAEIQATQYAAIYGYAQQNLILRTSSIYFNVLRAHQSVQSVQANKRAVARQLEQTKQRFDVGLIAITDVHEAQAEFDRTNADLIKAKNQLINSYYALRELTGEDIRNIDLLNTDIFSPAPLEGNVTLWHNNALEHNLLLHEKRIAKDLAQMQIKLARTGHQPTLDLSARIGYNNTDYDALRRSDIDMSDASIGIEFNLPIYTGGKTNSLVNQATYSYVMASEDLIQSFRSIDADIKSGYNNVRASISSITAYEQTVISSKSALEAIEAGFEVGTRTIVDVLDSTRRLFESEDLLANARYDYILSRLQLKYSAGTLSIQDIKDISAGLIKASDK

SEQ ID NO: 487 - protein, 497 aa, Rahnella sp.
MKKLLPLLIGLSLGGFSTISQAENLMQVYQQARTSNPDLRKSAADRDAAFEKINESRSPLLPQLGLGADYNYTNGYRDANGVNSDVGSASLQLTQTIFDMSKWRALTLQEKTAGIQDVSYQTDQQSLILNSATAYFNVLSAIDSLSYTEAQKQAIYRQLDQTTQRFNVGLVAITDVQNARSQYDTVLANEVTARNNLDNALESLRQVTGMYYPQLSSLNVDNFNTTRPQPVATLLKEAESRNLSLLSARLSQDLAREQIRYAETGHMPTVDLTASTGISNTKYHGDKTGGSSSYQDADAGQNKIGLSFSLPLYSGGSVTSQVKQAQYNFVGASEQLESAHRSVVQTVRSSFNNISASISSINAYKQAVVSAQSSLDAMEAGYQVGTRTIVDVLDATTTLYNAKQQLSSARYTYLINQLNIKSALGTLNESDLLALNGALGKPVPTAPDQVAPETPDQDAAADGYGNKTAAAPAPRVQQASASTATTTPKKSGNPFQN

SEQ ID NO: 488 - protein, 490 aa, Raoultella sp.
MKKLFPILIGLGLTGFSAMSQAENLMQVYQQARVSNPDLRKSAADRDAAFEKINEARSPLLPQLGLGADYTYTNGYRDSNGVNSNVTSGSLQLTQTLFDMSKWRALTLQEKAAGIQDVTYQTDQQTLILNTATAYFNVLSAIDTLSYTEAQKQAIYRQLDQTTQRFNVGLVAITDVQNARSQYDSVLANEVTARNNLDNAVEALRQVTGNYYPELASLNVDGFKTNKPQTVNALLKEAENRNLSLLQARLNQDLAREQIRQAQDGHLPTLDLTASTGVSNTSYSGSKTHNSTQYNDNDAGQNKIGLSFSLPLYQGGMVNSQVKQAQYNFVGASEQLESSHRSVVQTVRSSFNNINASISSINAYKQAVVSAQSSLDAMEAGYSVGTRTIVDVLDATTTLYNAKQQLSSARYNYLINQLNIKSALGTLNEQDLVALNNTLGKPVSTSPDSVAPETPQQDANADGYSSNAAPAATPASTRTTKTSGANPFRQ

SEQ ID NO: 489 - protein, 490 aa, Raoultella ornithinolytica
MKKLFPILIGLGLTGFSAMSQAENLMQVYQQARVSNPDLRKSAADRDAAFEKINEARSPLLPQLGLGADYTYTNGYRDSNGVNSNVTSGSLQLTQTLFDMSKWRALTLQEKAAGIQDVTYQTDQQTLILNTATAYFNVLSAIDTLSYTEAQKQAIYRQLDQTTQRFNVGLVAITDVQNARSQYDSVLANEVTARNNLDNAVEALRQVTGNYYPELASLNVDGFKTNKPQTVNALLKEAENRNLSLLQARLNQDLAREQIRQAQDGHLPTLDLTASTGISNTTYNGSKTNDPTRYGDTDAGQNKIGLSFSLPLYQGGMVNSQVKQAQYNFVGASEQLESSHRSVVQTVRSSFNNINASISSINAYKQAVVSAQSSLDAMEAGYSVGTRTIVDVLDATTTLYNAKQQLSSARYNYLINQLNIKSALGTLNEQDLLALNNTLGKPVSTSPDSVAPETPQQDANADGYSSNAAPAATPASTRTTKTSGANPFRQ

SEQ ID NO: 490 - protein, 491 aa, Salmonella sp.
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNAAQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPDQDAAADGYNAHSAAPAVQPTAARANSNNGNPFRH

SEQ ID NO: 491 - protein, 491 aa, Salmonella sp.
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNSAQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPEQDAAADGYNAHSAAPAVQPTAARANSNNGNPFRH

SEQ ID NO: 492 - protein, 491 aa, Salmonella sp.
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNSAQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPDQDAAADGYNAHSAAPAVQPTAARANSNNGNPFRH

SEQ ID NO: 493 - protein, 491 aa, Salmonella sp.
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNSTQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPEQDAAADGYNAHSAAPAVQPTAARANSNNGNPFRH

SEQ ID NO: 494 - protein, 492 aa, Salmonella sp.
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNSAQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPDQDAAADGYNAHSAAPAVQPTAARANSNNGNPFRHR

SEQ ID NO: 495 - protein, 486 aa, Salmonella sp.
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNSAQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPETPEQDAAADGYNAHSAAPAVQPTAARANSNNGNPFRH

SEQ ID NO: 496 - protein, 491 aa, Salmonella sp.
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAINALLKEAENRNLSLLQARLSQDLAREQIRQAQDGHLPTLNLTASTGISDTSYSGSKTNSTQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPEQDAAADGYNAHSAAPAVQPTAARANSNNGNPFRH

SEQ ID NO: 497 - protein, 494 aa, Salmonella bongori
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRALTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGVSDTSYSGSNTHGPGSQYDDSNMGQNKIGLNFSLPIYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPEQDAAADGYSANTAAPAVQPTAARTTSSKSGNPFHN

SEQ ID NO: 498 - protein, 491 aa, Salmonella enterica
MQMKKLLPILIGLSLSGFSTLSQAENLMQVYQQARLSNPELRKSAADRDAAFEKINEARSPLLPQLGLGADYTYSNGYSDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNSTQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPDQDAAADGYNAHSAAPAVQPTAARANSNNGNPFRH

SEQ ID NO: 499 - protein, 491 aa, Salmonella enterica
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNAAQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPEQDAAADGYNAHSAAPAVQPTAARANSNNGNPFRH

SEQ ID NO: 500 - protein, 491 aa, Salmonella enterica
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDITYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNSTQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPDQDAAADGYNAHSAAPAVQPTAARANSNNGNPFRH

SEQ ID NO: 501 - protein, 493 aa, Salmonella enterica
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDSVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSNTHGPGSQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPENVAPETPEQDAAADGYNAHSAAPAVQPTAARANSNNGNPFRH

SEQ ID NO: 502 - protein, 491 aa, Salmonella enterica
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNAAQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSKLGKPIPTSPESVAPETPDQDAAADGYNAHSAAPAVQPTAARANSNNGNPFRH

SEQ ID NO: 503 - protein, 491 aa, Salmonella enterica
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNSAQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESIAPETPDQDAAADGYNAHSAAPAVQPTAARANSNNGNPFRH

SEQ ID NO: 504 - protein, 493 aa, Salmonella enterica
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSNTHGPGSQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPDQDAAADGYNAHSAAPAVQPTAARANSNNGNPFRH

SEQ ID NO: 505 - protein, 491 aa, Salmonella enterica
MQMKKLLPILIGLSLSGFSTLSQAENLMQVYQQARLSNPELRKSAADRDAAFEKINEARSPLLPQLGLGADYTYSNGYRDANGINSNETSASLQLTQTLFDMSKWRGLTLQEKAAGIQDVTYQTDQQTLILNTANAYFKVLNAIDVLSYTQAQKEAIYRQLDQTTQRFNVGLVAITDVQNARAQYDTVLANEVTARNNLDNAVEELRQVTGNYYPELASLNVEHFKTDKPKAVNALLKEAENRNLSLLQARLSQDLAREQIRQAQDGHLPTLNLTASTGISDTSYSGSKTNSTQYDDSNMGQNKIGLNFSLPLYQGGMVNSQVKQAQYNFVGASEQLESAHRSVVQTVRSSFNNINASISSINAYKQAVVSAQSSLDAMEAGYSVGTRTIVDVLDATTTLYDAKQQLANARYTYLINQLNIKYALGTLNEQDLLALNSTLGKPIPTSPESVAPETPDQDAAADGYNAHSAAPAVQPTAARANSNNGNPFRH

SEQ ID NO: 506 - protein, 492 aa, Scandinavium goeteborgense
MKKLLPILIGLSLTGFSAVSHAENLLQVYQQARLSNPDLRKSAADRDAAFEKINEARSPLLPQLGLGADYTYTNGFRDNNGIDSNETSAALTLTQTIFDMSKWRALTLQEKSAGIQDVTYQTDQQTLILNTATAYFNVLSAIDSLSFTEANKQAVYRQLDQTTQRFNVGLVAITDVQNARSQYDTVLANEVTARNNLDNAVEQLRQVTGNYYPELSSLNVSTLKMDKPQPVNALLKEAEGRNLALLQARLSQDLAREQIRYAETGHMPTVGLTASTGVSDTSYDGSNATSSQYTDRNIGQNQIGLNFNLPLYSGGSVTSQVKQAQYNFVGASEQLESAHRNVVQTVRSSFNNINASISSINAYKQAVVSAQSSLDAMEAGYQVGTRTIVDVLDATTTLYNAKQQLSSARYSYLINQLNVKSALGTLNENDLLALNGTLGKAVSTSPEAVAPENAQQDANADGYNGASAAPAAQPAAQRTNTTSSNSSNPFKN

SEQ ID NO: 507 - protein, 505 aa, Serratia sp.
MKKLLPLLIGLSLGGFSAMSQAENLLQVYKQARESNPDLRKSAADRDAAFEKINEARSPLLPQLGLTAGYDYTNGYRDSNGVNSNVTSGSLALTQTIFDMSKWRQLTLQEKTAGISDVTFQTAEQQLILNTATAYFNVLKAIDTLSYTQAQKDAVYRTLDQTTQRFNVGLVAITDVQNARSNYDTVLAAEVSARNDLDNALETLRQVTGTFYPELASLNTDRFSTQRPEAVNNLLKEAEARNLSLLSARLSQDLAREQIRAAQTGYMPTIDVSASTGISNTKYNGSNTGGANAARYSDSDAGQNKVGISFNLPLYSGGATNSQVKQAQYGFVGASEQLESSHRSVVQTVRSSFNNVNASISSINAYKQAVISAQSSLDAMEAGYQVGTRTIVDVLDATTTLYNAKRQLSDARYTYLINQLNIKSALGTLNQNDLLLLNGALGKPVSTAPDAVAPQNRAQDAYADGYQDNAPMQQTAAPVPAATRASAPAVTTSQPARNSGNPFRN

SEQ ID NO: 508 - protein, 504 aa, Serratia sp.
MKKLLPLLIGLSLGGFSAMSQAENLLQVYKQARESNPDLRKSAADRDAAFEKINEARSPLLPQLGLSAGYNYNNGYRDSNGVNSNVTSASLGLTQTIFDMSKWRALTLQEKTAGISDVTFQTAEQQLILNTATAYFNVLSAIDTLSYTQANKQAVYRTLDQTTQRFNVGLVAITDVQNARSSYDTVLAAEVTARNNLDNALEALRQITGTFYPELASLNTDRFSTQRPEAVNNLLKEAEARNLSLLSARLSQDLAREQIRSAQTGYMPTVNLNASTGITNTRYNGSATHDTLSSSRYSNSDAGQNQVGVSISLPLYSGGATNSQVEQAQYGFVGASEQLESAHRGVVQTVRSSFNNVNASISSINAYKQAVISAQSSLDAMEAGYQVGTRTIVDVLNATSTLYNAKQQLSSARYTYLINQLNIKSALGTLNQNDLLTLNGALGKPVSTAPDAVAPQNRAQDAYADGYQDNAPMQQVAAPAPTATRAAAPAVSQPVRNSGNPFRN

SEQ ID NO: 509 - protein, 505 aa, Serratia sp.
MKKLLPLLIGLSLGGFSAMSQAENLLQVYKQARESNPDLRKSAADRDAAFEKINEARSPLLPQLGLTAGYDYTNGYRDSNGVNSNVTSGSLALTQTIFDMSKWRQLTLQEKTAGISDVTFQTAEQSLILNTATAYFNVLKAIDTLSYTQAQKDAVYRTLDQTTQRFNVGLVAITDVQNARSNYDTVLAAEVSARNDLDNALETLRQVTGAFYPELASLNTDRFSTQRPEAVNNLLKEAEARNLSLLSARLSQDLAREQIRAAQTGYMPTIDVSASTGISNTKYNGSNTGGANAARYSDSDAGQNKVGISFNLPLYSGGATNSQVKQAQYGFVGASEQLESSHRSVVQTVRSSFNNVNASISSINAYKQAVISAQSSLDAMEAGYQVGTRTIVDVLDATTTLYNAKRQLSDARYTYLINQLNIKSALGTLNQNDLLLLNGALGKPVSTAPDAVAPQNRAQDAYADGYQDNAPMQQTAAPVPAATRASAPAVTTSQPARNSGNPFRN

SEQ ID NO: 510 - protein, 500 aa, Serratia inhibens
MKKLLPLLIGLSLSGFSAMSQAENLLQVYKQARESNPDLRKTAADRDAAFEKINEARSPLLPQLGLSAGYNRSYGYRDSRDMNSNTTSGSLALTQTIFDMSKWRALTLQEKTAGISDVTFQTSSQQLILDTATAYFNVLSAIDSLSYTQAQKQAVYRTLDQTTQRFNVGLVAITDVQNARSNYDTVLASEVTARNTLDNALEKLRQVTGTFYPELASLNTDSFNTKRPEAVNNLLKEAEARNLSLLSARLSQDLAREQIKSAQTGYMPTVDFSASTAVSNNRYSGSNNLSQDNDAGENKVGLSFNLPLYSGGQTNSQVQQAQYNFVGASEQLESAHRSVVQTVRSSFNNVNASISSINAYKQAVVSAQSSLDAMEAGYQVGTRTIVDVLDATTTLYNAKQQLSSARYTYLINQLNIKSALGTLNENDLLLLNGALGKPVSTSQDVVAPPTSAQDAYADGYNDNAPASQTAAPAPVATRASAPAITTTQPARSDSGNPFRN

SEQ ID NO: 511 - protein, 500 aa, Serratia liquefaciens
MKKLLPLLIGLSLGGFSAMSQAENLLQVYKQARESNPDLRKTAADRDAAFEKINEARSPLLPQLGLSAGYTHTNGYRDSRDQNSDATSGSLALTQTIFDMSKWRALTLQEKTAGISDVTFQTSSQQLILDTATAYFNVLSAIDSLSYTQAQKQAVYRTLDQTTQRFNVGLVAITDVQNARSNYDTVLAAEVTARNTLDNALEKLRQVTGTFYPELASLNTDRFSTQRPEAVNNLLKEAESRNLSLLSARLSQDLAREQIRSAQTGYMPTIDFNASTAVSNTKNNGSRNSTPDADIGQNKVGLSFNMPLYSGGQTNSQVQQAQYNFVGASEQLESAHRNVVQTVRSSFNNVNASISSINAYKQAVVSAQSSLDAMEAGYQVGTRTIVDVLDATTTLYNAKQQLSSARYTYLINQLNIKFALGTLNENDLMLLNGALGKPISTSQDVVAPPTTAQDAYADGYQDNAPAQQSAAPAPVAATRTSAPVSTTTPPVRNSGNPFRN

SEQ ID NO: 512 - protein, 500 aa, Serratia plymuthica
MKKLLPLLIGLSLSGFSAMSQAENLLQVYKQARESNPDLRKTAADRDAAFEKINEARSPLLPQLGLSAGYNRSYGYRDNRDQNGNTTSGSLALTQTIFDMSKWRALTLQEKTAGISDVTFQTSSQQLILDTATAYFNVLSAIDSLSYTQAQKQAVYRTLDQTTQRFNVGLVAITDVQNARSNYDTVLASEVTARNTLDNALEKLRQVTGTFYPELASLNTDRFSTQRPEAVNNLLKEAEARNLSLLSARLSQDLAREQIKSAQTGYMPTVDFSASTAVSNNRYSGSRSISTDNDAGENKVGLSFNLPLYSGGQTNSQVQQAQYNFVGASEQLESAHRSVVQTVRSSFNNVNASISSINAYKQAVISAQSSLDAMEAGYQVGTRTIVDVLDATTTLYNAKQQLSSARYTYLINQLNIKSALGTLNENDLLLLNGALGKPISTSQDVVAPPTTAQDAYAEGYNGNAPAPQTAAPAPVATRASAPAITTTQPARSNSGNPFRN

SEQ ID NO: 513 - protein, 483 aa, Serratia symbiotica
MKKLLPFLIGLSLGGFSAMSQAENLLQVYKQARESNPELRKTAADRDAAFEKINEARSPLLPQLGLTAGYAYGRGYRDANNINSTMTNGSLALTQTIFDMSKWRVLTLQEKTAGISDVTFQTSSQRLILDTATAYFSVLSAIDALSYTQAQKQAVYRTLDQTTQRFNVGLVAITDVQNARSNYDTVLASEVTARNTLDNALEKLRQVTGTFYPELASLDTDRFNTQRPEAVNNLLKEAEAHNLSLLSARLSQDLAREQIRLAQTGYMPTINMSASTGITDTKYSGSSSSGDTDSNTGQNKIGLSINLPLYSGGATNSQVQQAQYSFVGASESLESAHRSLVQTVRSSFNNVNASISSINAYKQAVVSAQSSMDAMEAGYQVGTRTIVDVLNATTTLYNAKQQLSSARYTYLINQLNIKSSLGTLNENDLLALNGALGKPVSTSPELLAPATTEQNAYANGYNGDGLATDTTQPARNSGDPFRH

SEQ ID NO: 514 - protein, 435 aa, Shewanella sp.
MKFKIHNLCAALTLAASASAVQADDLLQIYQQALTSDPLVLQAQAQRNGLFEQIEQNRAPLLPTISANVGYDKAWNDPKDDSSGLVGSLKLNQVIYDHSAWVGLSLAEKAASQADSAYASALQNLITRITTAYFNVLTAKDNYEFQGAEKRAIERQLEQTKQRFAVGLTAITDVHEAQAQYDLASATEILAENTLANSYEALREITGIDHKTINVLDTNRFSAVTPAPTSSNEWLKIAETNSVDLMTQRIGKDIAQETIKLYQAGHMPSLSLNAGYNKGLEQKTGNINEPDFDNVSVGVNLSIPIFEGFKVSSQVNQAQFQYVEASEKLEQVYRGVVKNVRNNYNNVGASISSIKAYEQSVISSESALKATQAGFEVGTRTIVDVLNRTRDLYDSKRKLSDARYSYINSVIALKQAAGTLNEDDVIAINNGLIAE

SEQ ID NO: 515 - protein, 435 aa, Shewanella sp.
MKFKIRSLCAALTLAASAQAVQADDLLQIYQQALTNDPLVLQAQAQRDALFEKIEQNRAPLLPTLSANVGYDKAWNDPRDNTSGLTGGLKLNQVIYDHSAWVGLSLAEKAASQADSNYASTLQTLITRVTKAYFDVLTAKDNYEFQGAEKRAIERQLEQTKQRFAVGLTAITDVHEAQAQYDLASASEILAENDLANSYEALREITGIDHKTLNVLDTNRFSAVTPAPSSPSEWLKIAESNSVDLMTQRIGKDIAQETISLYKAGHMPSLSLSAGYNKGLEQKTGSVNDPDFDNVNVGLNLSIPIFEGFKVTSQVNEAQYQYVEASEKLEQTYRSVVKNVRNNYNNVGASISSIRAYEQSVISSESALKATQAGFEVGTRTIVDVLNRTRDLYDSKRKLSDARYSYINSIVALKKAAGTLNEDDVIAINSGLKAE

SEQ ID NO: 516 - protein, 435 aa, Shewanella sp.
MKFKIRSLCVALTLAASAPAVQADDLLQIYQQALTSDPLVLQAQAQRNALFEKIEQNRAPLLPTISANVGYDKAWNDPRDDSSGLTGNLKLNQVIYDHSAWVGLSLAEKAASQADSTYASALQNLITRVTKAYFDVLTAKDNYEFQGAEKRAIERQLEQTKQRFAVGLTAITDVHEAQAQYDLASASEILAENTLANSYEALREITGIDHKALNVLDTNRFSAVTPAPSSSSDWLKIAETNSVDLMTQRIGKDIAQETISLYKAGHMPSLSLNAGYSKGLEQKLGNSNEPDFDNMSAGINLSIPIFEGFKVSSQVKEAQFQYVEASEKLEQTYRSVVKNVRNNYNNVGASISSIRAYEQSVISSESALKATQAGFEVGTRTIVDVLNRTRDLYDSKRKLSDARYSYINSIVALKQAAGTLNEDDVIAINNGLKAE

SEQ ID NO: 517 - protein, 438 aa, Shewanella algae
MKFKIRTLCAALTLAVSAPAVQADDLLQIYQQALTNDPVVLQAKAQRDQLYEKIEENRAPLLPTISANVGYDKAWNDPSADTSGLTGAIKLNQVIYDHSAWVGLSLAEKAASQADAVYASALQTLITRVTTAYFDVLAAKDNYEFKGAEKRAIERQLEQTKQRFAVGLTAITDVHEAQAQYDLATADEILAENQLINSYEALREITGIDHKSIDILDTNRFSAASPTPAVPGDWLKMAETNSVDLLTQRIGKDIAQETISLYKAGHMPSLSLSAGYTKGLEQEIGSQSQPDFDNGTVGVNLSVPIFEGFKVSSQVKQAQYAYVEASQKLEQTYRQVVKNVRNNFNNVGASISSIKAYEQSVLSSESALKATQAGFEVGTRTIVDVLNRTRDLYDSKRQLSNARYGYIKSILALKQATGTLNEDDVISINNGLTAPAQQ

SEQ ID NO: 518 - protein, 448 aa, Shewanella benthica
MKFKIRTICAALTLAFSTSALQADDLLQIYQQALTSDPIALQAQARRDALYEQIEENRAPLLPTISASVGYNKSWHNEDTMSNTDSSGINAGVTLNQVIYDHSAWVGLSLAELAASQADAAYASSLQTLIIRVTSAYFAVLSAKDTFEFQGSEKRAIERQLKQTKQRFAVGLTAITDVHEAQAQYDLARAQEILAENELTNSYEALREITGIEHRTINILDTDRFSAVSPSPTRAIDWLKIAETNSVDLLTTRIGKDIAKETISLYKAGHMPTLSLNAGYTTNIQQENNDNDANDFDNGTVGLTLSIPIFEGFKISSKVNQAQYQYVEASEKMEQVHRKVVKDIRNNFNNVGASISSIRAYEQSVISSESALKATQAGFEVGTRTIVDVLNRTRDLYDSKRKLSDARYGYINSILALKQAAGTLNEDDVITINQGLIAQAQATQNTTQ

SEQ ID NO: 519 - protein, 435 aa, Shewanella oneidensis
MKFKIRSLCVALTLAASAHAVQADDLLQIYQQALTNDPLVLQAQAQRNALFEKIEQNRAPLLPTISANVGYDKAWNDPRDDSSGLTGSLKLNQVIYDHSAWVGLSLAEKAASQADSTYASALQNLISRVTKAYFDVLTAKDNYEFQGAEKRAIERQLEQTKQRFAVGLTAITDVHEAQAQYDLASASEILAENTLANSYEALREITGIDHKALNVLDTNRFSAVTPAPSSSTDWLKIAETNSVDLMTQRIGKDIAQETISLYKAGHMPSLSLNAGYAKGLEQKLGDASEPDFDNMSVGINLSVPIFEGFKVSSQVKEAQFQYVQASERLEQTYRGVVKNVRNNYNNVGASISSIRAYEQSVISSESALKATQAGFEVGTRTIVDVLNRTRDLYDSKRKLSDARYSYINSIVALKQAAGTLNEDDVIAINNGLKAE

SEQ ID NO: 520 - protein, 442 aa, Shewanella sp.
MKFKIRTICAALTLAVTTSAVQADDLLQIYQQALTSDPIALQAQAQRDTLYQQIEENRAPLLPTISASVGYGKSWHNESSADTDSTGLNAGVTLNQVIYDHSAWVGLSLAELAASQADAAYASSLQNLIIRVTSAYFEVLSAKDTFEFQGSEKRAIERQLEQTKQRFAVGLTAITDVHEAQAQYDLARASEILAENQLINSYEALREITGIDHKSINILDTNRFSAVTPSPAKSNDWLKMAESNSVDLLTTRIGKDIAQETISLYKAGHMPSLNLNAGYKTNIQQENQNGSQPDFDNANIGITLSVPIFEGFKVSSKVNQAQYQYVEASEKMEQAYRKVVKDVRNNFNNVGASISSIRAYEQSVISSESALKATQAGFEVGTRTIVDVLNRTRDLYDSKRQLSDARYGYINSIMALKQAAGTLNEDDVIAINNGLIAEAVTP

SEQ ID NO: 521 - protein, 493 aa, Shigella flexneri
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLEREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 522 - protein, 457 aa, Simiduia agarivorans
MFSRTPLAASLRWICAAGLLSAAPLSMADTLNQIYELAVKNDHQIKADEAAYRSAREAKTIARANLLPLISANASYSQSDQDITSSGVGPVPTKTNVQSWGATLAQPLLNMPAWFGYKQGEELTNKAEADFSAAQQDLIVRTATAYFNVLRAIDTLEASIAEENANSHQLAQTKQRFEVGLTAITDVHEAQASYDIATAQTLTDRGNLGIAFEALEVLTGKPHDKVAPLKEEFPVLKPQPEARSEWVEFALQNNYSLKSAKFASEASRRGAQASQAAHYPTLTASLSYNDSDRDIQDTILGDNVTTGSSAAISLNVPIYEGGGTSARSRQAAENAMQSQELFNKTQRDTIQAARSLHLQVTTDVARVAARKQAITSSLSAVQATQAGYEVGTRNLVDVLQVQRTLYQARRDYSNSLYDYVINLFRLKAVAGNLTPADVVQINQWLDEAAAQNRAKYE

SEQ ID NO: 523 - protein, 450 aa, Spiribacter salinus
MRHNIRLHRPLALLITIGLASAPLGVSAEDTLIEIYDRARQSDPQFQQAIADRRAREEALPQARAGLRPDVSLSSRYTTRDTDSDIGTTTGSEEEDYRQLSYGVELTQPLYRFSQARSVDRADALVEQARADFASAEQALIIRTAERYFSVLDARESVDAAAANLEAIERQLEQAEQRFEVGVIARTDVEEARAQADLARAELLQAEDDFETERERLRELTDRAPARLRQVREGVELNAPSPNDPDAWRGRAEDENRDLAAARFAAEAAMEGVDVERGGRFPQIDLIAGYDGLEQYDRDGQDRSSEEYSAGIQLNLPLYQGGGVSSSIREAQFRYTEAREALEEARRTVTRNAADAYRGVETALERVRALEQARISTQAALDATEAGFEVGTRTIVDVLNAQREVFNAERDYQQARHAYLVNTLRLQQAAGALSAEDLRGVDTLLGNGNG

SEQ ID NO: 524 - protein, 452 aa, Stenotrophomonas sp.
MIRRSLAVALATALLPLSAHAADLLQVYEMARNGDPQLSAAESTRLVDKEGAVQARAALLPQINGQATLNRTRSEPNADINSGSFTNKRRNYTIDGSQTLFNWTQINNLRSQRELSKAADFTLESANDSLIVRTSAAYFNVLVAIESLNAAQTNEAAAKKQFDFADKRLEVGLAPITDVHEARAQYDQARANTIVAQNTLADNYQALTELTGQPVVNLRGLPADFRPEVPANRGNIDELVHQATTQNPALKAQELKVSAAEAGVQAARGGHYPTLSLGGSWGKTATWGDSTGSGSLSPDARTNSIGLTLNVPIFAGGATQSGVRQALAQRDIAQDGYEQQKRALDRNTRNAYQTLVQGISEVEARRLAVVSAQSAYDASQVGLEVGTRTVLDVIQNQRILFSAQLDYAQARYTFLQNRLLLSQSLGALDVAELQDVNRLLTQDAGNPATTTH

SEQ ID NO: 525 - protein, 452 aa, Stenotrophomonas sp.
MIRRSLAVALATALLPLSAHAADLLQVYEMARNGDPQLSAAESTRLYDKEGAVQARAALLPQINGQAQLNRTRSEPNADANSGSFTNKRRTYTIDGSQTLFNWTQINNLRSQRELSKAADFTLDSANDSLIVRTSAAYFNVLVAIESLNAAQTNEAAAKKQFDFADKRLEVGLAPITDVHEARAQYDQARANTIVAQNTLADNYQALTELTGQPVTNLRGLPADFRPEVPANRGNIDELVQQATTQNPALKAQELKVSAAEAGVQAARGGHYPTLSLGGSWGKSATWGDSTGSGSLSPDARTNSIGLTLSVPIFSGGATQSGVRQALAQRDIAQDGYEQQKRALDRNTRNAYQTLVQGISEVEARRLAVVSAQSAYDASQVGLEVGTRTVLDVIQNQRILFSAQLDYAQARYTFLQNRLLLSQSLGALDVAELQDVNRLLTQDAGNPATTTH

SEQ ID NO: 526 - protein, 439 aa, Thioalkalivibrio nitratireducens
MKMLRALCATAVFAATWALPAQAEDLLQVFEHAQMEDAQLRAAEAQYRAVLEARPIARSTLLPQLSADAELGAFYSDPDGSSSIDGSSHSIGLNLNQSLFDQRNRIGVRQADLQISSAAAELDAARQDLILRVAEAYFGVLVARETLEFRRAEREAISRQLEQTQRRFEVGLIAITDVKEAQAQFDIASAEQIAAENALNLAREQLAVITNRYYDQLAALGEHLDMPSPDPMDPQDWVSAALENNQELNAQRLSAEVAREQIGRQRAEGLPTLGLGASVSDTGYSGVNSVPGGQFNDRTDAQIGLRLDVPLYTGGRVSAITREAREQFEAAQETVVFTQRQTVQNTRNSYLSVIANASRARALAQALQSTQAAFESAQAGFEVGTRTQVDVLLALREVFRAERDYAEARYGYLLETLRLQRAVGSLSLADLQRINAFLE

SEQ ID NO: 527 - protein, 480 aa, unclassified Pseudomonas
MLRKLSLAIAVSCASNGMAWAAEAPLTTTKTDLVSVYQEAVDNNADLAAARAQYGAQKEVVPQARAGLLPNLSAGADLNNTRTKLDEPAMTANRSGNVYRATLAQPLFRADRWFQLQAAKEVNEQAALQLSATEQNLILQSAENYFAVLRAQDNLASTKAEEAAFKRQLDQSNERFDVGLSDKTDVLQSQASYDTARANRIVAQRQVDDAFEALITLTNREYNSIQGIVHTLPVLAPVPNDAKAWVDTAAKQNLNLLASNYAVSAAEETLKQRKAGHAPTLDAVVQYKKGDNDALGFSNPNPNPIAPPYHGDVEQRSIGLQLNIPIYSGGLTSSQVRESYSRLSQSEQQRESLRRQVVENTRNLHRAVNTDVEQVQARRQSIISNQSAVEATEIGYQVGTRNIVDVLDAQRQLYNSVRNYNNSRYDYILDNLRLKQAAGTLNPGDLQDLARYLKADYNPDRDFLPPDLAKAAAEQLKARP

SEQ ID NO: 528 - protein, 477 aa, unclassified Pseudomonas
MLRRHSVIVAVSYIFSSAACAANTPTTVNNDLVSVYEEAVDNNADLAVARADYSARREVVPQARAGLLPNLSAGAEMMNTRTKLDEPSVTSNRSGNSWSATLAQPIFRADRWFQLQAAEAVNEQAALQLSATEQSLIRQTAETYFAVLRAQDNLASTKAEESAFKHQLEQSNERFDVGLSDKTDVLQSQASYDTARANRIVAERQVQDAFEALVTLTNRQYTAIQGVVHTLPVQVPTPNDAKAWVEAAGRQNLNLLASNHAVTAAEETVRQRKAGHAPTLDAVVKYQKGDNDSLGFTNPSQTGVRYGGDVEQTSVGLQLNIPIYSGGLTSSQVREAYSRLTQSEQQRESLRRQVVESTRNLHRAVNTDVEQIQARKQSIVSNQSALEATEVGYQVGTRNIVDVLDAQRQLYASVRDYNNSRYDYILDNLRLKQAAGTLSPQDLRDLGRYLKVNYNPDKDFLPPDLAVEAKKNFGSRP

SEQ ID NO: 529 - protein, 475 aa, unclassified Pseudomonas
MHLSPRALAGCLLILLSPFAGLVRATEPQAMPSDLWRVYQDARHNNSELAAARADQAARAEAVPQARAGLLPTLSASAEHNGTSTSLQQPRQATRRSGTSYQAVLNQPVFRAERWFGLKAAEAEDQQAMLELAAAEQKLMLDSAQAYFGLLKAQDALAAATAEEAALKRQLALAQRGLQLGLSDRTDVLQAEAGHDTAQANRIVARKRTDDAFEALDTLTHQQYPALQGLRHDMPVLLPEPNDARRWVDSAVRQNLTLQASQYALEATQQTLKARKAGHAPTLDAVLRYQVGDNDNLGYGNSDIRGSGYGGDVQQRSVGLQLNIPLFSGGQTSSQVREAYQRMSQRESLNDNLRRQVVEQTRNLHRGLNSGADQVRARRQSIISNQGAVLASQLGLQVGTRNIVDVLEAQRQLYNAVRQYNDSRYDYILDTLRLKQAAGTLSPEDLKALCDYLKADYDPDRDFLPPEFPRRLAAR

SEQ ID NO: 530 - protein, 480 aa, unclassified Pseudomonas
MLRRLSLAIAVAAASVGLVQAAEAPLSSKTDLVTVYQEAAKNNADIAAARADYQARREVVPQARAGLLPNLSAGANYGDTRTEIDSPSATISRSGLVYQANLSQPLFRADRWFQLQAAEATSEQAALELSATEQNLILQSAETYFAVLRAQDNLASTRAEESAFKRQLDQANERFDVGLSDKTDVLEAQAGFDTARANRLLAERAVDDAFEALVTLTNRDYVAVEGIVHTLPVLAPTPNDAKSWVDTAAAQNLNLQASLYAVNAAEENLRQRKAGHAPTLDAVASYQKGDNDSLGFTNSGSPLSPSYSGDVSQRTIGLQLNIPLYSGGLTSSQVREAYQRLGQTEQLRESLRRQVVQNTRNLFRAVNTDVETVQARRQSIISNQSALEATEIGYQVGTRNIVDVLDAQRQLYSAVRNYNDARYDYILNNLRLKQAAGTLSPADLEALGSFLKPDYNPDKDFLPPDLASAAEERLKNNQDF

SEQ ID NO: 531 - protein, 509 aa, unclassified Sodalis
MKKLLPLLIGLSLGGFSTLSQAENLLQVYQQARLSNPDLRASAADRDAAFEKINEARSPLLPQLGLGVDYTYNNGFRDQRDINSNETSGSLQLTQTIFDMSKWRALTVQEKTAGVQDVTYQTQQQTLILNTATAYFNVLSAIDALSYTEAQKQSIYRELDQTTQRFNVGLVAITDVQNARSNYDTVLADEVNARNTVDNALESLRQISGNFYPQLASLNIDRFKAAQPQPINALLKEAEMRNLTLLSARLSQDVAREQIRLEETGHYPTLALTASSSLSNTDYNGSRTNGNANYNNTNIGQNAIGLSLSVPIYSGGAVTSQVKQAQYSFVSASEQLESAHRSVVQTVRSSFNNISASISSIDAYRQAVISAQSSLDASEAGYEVGTRTIVDVLDATTTLYDAKRNLSTARYNYVINQLTLKNALGTLNEDDIRTLNASLGQPIATSPESVAPVNPNQDANAEGVNPGQTSRAPRSNPSDAAQSARQSATPVSATVPASSSAKKRNPFQH

SEQ ID NO: 532 - protein, 442 aa, Vibrio sp.
MKKLLPLFISAALGSMSTSAFADSLAEIYDLAKQNDPQLLSVAAQRDQAFEAITSSRSALLPQINLTAGYNLTRGDTEYDSNLISDVSNDGNALTAGVSFSQALYNRASWISLDTAEKSARQADATYAATQQALILRVSQAYFEVLRAQDNLVFVRAEKAAVGRQLEQTKQRFEVGLSAITDVHDAQAQYDQVLADEVLAENALTNSYESLREITGQEHKDLNILDTGRFTASPSTVPAESLIDEAKTKNLTLLSSRILQDIARDNISLASSGHLPTLSLDGGYNYGDTSHSSRDNTTDNFNIGVNLAVPLYTGGNVTSQTKQAEYAYVAASEDLEAQYRSVVKDVRAQNNNINASIGALRAYEQAVISARSALEATEAGFDVGTRTIVDVLDATRRLYDANKNLSNARYDYILSVLQLRQAVGTLSEQDILDVDAGLKAAK

SEQ ID NO: 533 - protein, 440 aa, Vibrio sp.
MKKLLPLFISAALGSMSTSAWADSLAEIYDLAKQNDPQLLSVAAQRDQAFEAINSSRSALLPQINLTAGYDITRGDRDLDAGGKTSTDENVLSAGVGFTQELYNRASWITLDTAEKTARRADANYAATQQGLILRVSQAYFEVLRAQDNLVFVRAEKAAVGRQLEQTKQRFEVGLSAITDVHDAQAQYDAVLADEVLAENSLINSYESLREITGQEHKNLNVLDTARFSASRSSEAAETLIEEAKTTNLSLLSARIGQDIARDNISLASSGHLPTLSLDGGYNYADTSESVTDNTTDGFKVGVNLAVPLYTGGRTTSEVKQAEYGYVSASEELEKEYRSVVKEVRAQNNNINASIGALKAYEQSVVSAQSALEATEAGFDVGTRTIVDVLDATRRLYDANKNLSDARYNYILAGLQLRQAVGTLSEQDILDVDAGLKPAK

SEQ ID NO: 534 - protein, 442 aa, Vibrio sp.
MKKLLPLFISAALGGISSSAWADSLAEIYDLAKQNDPQLLSVAAQRDRAFEAITSSRSALLPQINLTAGYNLTRGDTEYDSNLISDVSNDSNALTAGVNFSQELYNRASWITLDTAEKSARQADATYAAAQQGLILRVSQAYFEVLRAQDNLVFVRAEKAAVGRQLEQTKQRFEVGLSAITDVHDAQAQYDAVLADEVLAENDLINSYESLREITGQEHKNLNVLDTNRFSATRTNSPAETLIDEAKTKNLSLLSARISQDIARDNISLASSGHLPTLSLDGGYNYGDTSNSARDNTTDNFNIGVNLAVPLYTGGNVTSQTKQAEFAYVAASEDLEAQYRSVVKDVRAQNNNINASIGALKAYEQSVVSARSALEATEAGFDVGTRTIVDVLDATRRLYDANKNLSDARYNYILSVLQLRQAVGTLSEQDILDVDAGLKPAK

SEQ ID NO: 535 - protein, 454 aa, Vibrio sp.
MKKLLPLFISAALGGISSSAWADSLAEIYDLAKQNDPQLLSVAAQRDRAFEAITSSRSALLPQINLTAGYNLTRGDTDYDDSALGKSSNDQDALTAGVSFSQTLYNRASWISLDTAEKSARQADATYAATQQGLILRVSQAYFEVLRAQDNLVFVRAEKAAVGRQLEQTKQRFEVGLSAITDVHDAQAQYDAVLADEVLAENDLINSYESLREITGQEHKNLNVLDTNRFSATRTSSPAETLIEEAKTKNLSLLSARISQDIARDNISLASSGHLPTLSLDGGYNYGDYSGTESNYNSGLGRNANSDRTSDGFNIGVNLAVPLYTGGNVTSQTKQAEFAYVAASEDLEAQYRSVVKDVRAQNNNINASIGALKAYEQSVVSARSALEATEAGFDVGTRTIVDVLDATRRLYDANKNLSDARYNYILSVLQLRQAVGTLSEQDILDVDAGLKPAK

SEQ ID NO: 536 - protein, 435 aa, Vibrio sp.
MKKLLPLFISAALGSLSSSVWADTLTEIYNQAKENDPQLLSSAAQRDAAFEAVTSSRGDLLPQINLTAGYNINRSDIDARESDKLTAGISFSQQLYQRSSWVSLDTAEKNARKADSAYAATQQGLILRVAQAYFEVLRAKDNLAFVRAEKAAVARQLEQTKQRFEVGLSAITDVHDAQAQYDGVLADEVLAKNSLTNSYETLREITGQEHSDLSVLDTNRFSASKTTQPIDALLEEAQQKNLSLLTARIAQDVAKDNISLASSGHLPSLTLDGGYKYGDESNNNSGSKGDYNDFNVGINLAVPLYTGGKTTSQTKQAEFAYVAASQDLEKTYRSVVKDVRAFNNNINASIGALRAYEQSVISAKSALEATEAGFDVGTRTIVDVLDSTRRLYDANKNLSNARYDYVLSVLQLRQAVGTLSEQDILDINAGLKADS

SEQ ID NO: 537 - protein, 454 aa, Vibrio alginolyticus
MKKLLPLFISAALGSMSTSAFADSLAEIYDLAKQNDPQLLSVAAQRDQAFEAITSSRSALLPQINLTAGYNLTRGDNDYNDGALGKSSNDQDALTAGVSFSQTLYNRASWISLDTAEKSARQADATYAATQQALILRVSQAYFEVLRAQDNLVFVRAEKAAVGRQLEQTKQRFEVGLSAITDVHDAQAQYDQVLADEVLAENALTNSYESLREITGQEHKDLNILDTGRFTASPSTVPAESLIDEAKTKNLTLLSSRILQDIARDNISLASSGHLPTLSLDGGYNYGDYSGTDNTFNSGLNRNANSDRTSDNFNIGVNLAVPLYTGGNVTSQTKQAEYAYVAASEDLEAQYRSVVKDVRAQNNNINASIGALRAYEQAVISARSALEATEAGFDVGTRTIVDVLDATRRLYDANKNLSNARYDYILSVLQLRQAVGTLSEQDILDVDAGLKAAK

SEQ ID NO: 538 - protein, 449 aa, Vibrio aphrogenes
MKKLLPLFISLSLGGIHLAHADDLAQIYNQAKENDPQLLSSKADRDAAFEAIESSRGDLLPQINLTAGYNINRNINSHNNDPVESYGASTSEQNVFTAGLDFSQQLYSRDSWVNLDIAEQNARQQDSAYAAAQQEMILRVSQAYFDVLQAQDNLTFIQAEKKAVGRQLEQTKQRFEVGLSAITDVHDAQAQYDSVLADEIIAQNDLTNSYEALREITGQDNKDLRVLDTKRFTASTPQTTQASLVDEAKQKNLSLLTARIAQDIAKSNISLAQSGYSPSLTLDGGYQYSDIDEHNSHSPYSNYTSNDYNIGVNLVIPLYQGGSTSADVKTAQYQYVSTSQQLEETYRSVVKDVRAYYNNINASIGTIRAYQQSVISAKSALEATEAGYEVGTRTIVDVLDSTRSLYDANRNLSDARYNYILSVLQLKQAVGTLSEQDVLDINAGLIAAK

SEQ ID NO: 539 - protein, 446 aa, Vibrio casei
MKKLLPLLISLSLGGTHLAHADDLAEIYNQAKENDPLLLSSKADRDAAFEAINSSRGVLLPQINLTAGYNITRNIDSHDSHRPSAYTDENNNFTAGLDFSQQLYQRDSWVNLDIAEQNARQQDSSYASQQQSLILRVSQAYFGVLQAQDNLSFVSAEKKAVGRQLEQTKQRFDVGLSAITDVHDAQAQYDSVLADEILAQNDLTNSYEELREITGQDNKDLRILDTKRFSASTPQTTQNSLVDEAQTKNLDLLTARIAQDIAKSNISLANSGYTPSLTLDAGYQYSDVDAHNDHSSLNNYKSNDYNVGINLVVPLYQGGSTSADVKTAQYQYVSASQQLEATYRGVVKNVRAYYNNINASIGTIRAYQQSVVSAKSALEATEAGYEVGTRTIVDVLDSTRSLYDANRNLSDARYNYILSVLQLKQAVGTLSEQDILDINTGLVAAK

SEQ ID NO: 540 - protein, 438 aa, Vibrio cholerae
MKKLLPLFVSAALGTLSSAVWAENLAEIYNQAKENDPQLLSVAAQRDAAFEAVTSSRSALLPQINLTAGYNINRSDQAPRESDLLSAGINFSQELYQRSSWVSLDTAEKKARQADSQYAATQQGLILRVAKAYFEVLRAQDNLEFVRAEKAAVGRQLEQTKQRFEVGLSAITDVHDAQAQFDGVLADEVLAENSLTNSYEALREITGQEYSKLAVLDTKRFAASRTTESSEALIEKAQQQNLSLLAARISQDVARDNISLASSGHLPSLTLDGGYNYGNNSNDNAKNTSGEEYNDFKIGVNLKVPLYTGGNTTSLTKQAEFAYVAASQDLEAAYRSVVKDVRAYNNNINASIGALRAYEQAVISAKSALEATEAGFDVGTRTIVDVLDATRRLYDANKNLSNARYDYILSVLQLRQAIGTLSEQDVMDVNAGLKVAKK

SEQ ID NO: 541 - protein, 438 aa, Vibrio cholerae
MKKLLPLFVSAALGTLSSAVWAENLAEIYNQAKENDPQLLSVAAQRDAAFEAVTSSRSALLPQINLTAGYNINRSDQDPRESDLLSAGINFSQELYQRSSWVSLDTAEKKARQADSQYAATQQGLILRVAKAYFEVLRAQDNLEFVRAEKAAVGRQLEQTKQRFEVGLSAITDVHDAQAQFDGVLADEVLAENSLTNSYEALREITGQEYSKLAVLDTKRFAASRTTESSEALIEKAQQQNLSLLAARISQDVARDNISLASSGHLPSLTLDGGYNYGNNSNDNAKNTSSEEYNDFKIGVNLKVPLYTGGNTTSLTKQAEFAYVAASQDLEAAYRSVVKDVRAYNNNINASIGALRAYEQAVISAKSALEATEAGFDVGTRTIVDVLDATRRLYDANKNLSNARYDYILSVLQLRQAIGTLSEQDVMDVNAGLKVAKK

SEQ ID NO: 542 - protein, 444 aa, Vibrio coralliilyticus
MKKLLPLFISAALGSLSTNAFADTLTEIYNQAKENDPTLLSAAATRDSAFEAVTSSRATLLPQISLTAAYDINRGERNSETNDSDAWNAAVGFTQELYKRSSWITLDTAEKTARRADATYAAAQQALILRVATAYFDVLKAQDNLVFVQAEKAAVGRQLEQTKQRFEVGLSAITDVHDAQAQYDSVLADEVLAQNDLINSYEGLREITGLEHTNLNILDIARFSASKTDAPVDALVEEAQQKNLSLLSSRIAQDIAKDNISLQSSGHLPSLTLDGNYTLAEQMNSTPSSGDFDSDGFNVGLNLSVPLYTGGSTTSLTKQAEFDYVAASQDLEATYRSVVKDVRAFNNNISASIGALRAYEQTVVSAKSALEATEAGFDVGTRTIVDVLDSTRNLYDANKNLSNARYDYILSVLQLRQAVGTLSEQDILDINAGLKEAPANTANS

SEQ ID NO: 543 - protein, 438 aa, Vibrio fluvialis
MKKLLPLLISATLGGLSTTASADDLAQIYDQAKQNDPQLLSAAAQRDAAFEAINSSRSSLLPQINLTAGYNINRSDVDLRDSDKLSAGINFSQELYDRSSWVSLDTAEKQARQADAQYANSQQSLMLRVAQAYFDVLSAQDNLEFVRAEKAAVGRQLEQTKQRFEVGLSAITDVHDAQAQYDTVLADEVLAENSLINSYESLREITGQEHTNLSILDTNRFSTSRTTESMEALIEQAQEKNLSLLSARISQDVAKDNISLASSGHLPSLTLDGGYNYGREYNDNYSSYDTYHENNDFNIGLNLTVPLYSGGNVSSQTKQAEYAYVAASQDLEASYRSVVKNVRAYNNNINASIGSVRAYEQSVISAQSALDATEAGFDVGTRTIVDVLDATRTLYSVKKNLSDARYSYIINVLQLRQAVGTLSEQDIIDVNAGLKAIK

SEQ ID NO: 544 - protein, 441 aa, Vibrio kanaloae
MKKLLPLFISAAIGSLSSSAFADTLAEVYDQAKQNDPQLLRSAAQRDAAFEAVTSSRSDLLPQINLTANYDINRGDRDTSGTGSSSIDNNTWGAAIGFTQELYQRSSWITLDTAEKTARQFDSAYAAEQQALILRVSTAYFEVLRAQDNLEFVRAEKAAVARQLEQTKQRFEVGLSAITDVHDAQAQYDGVLADEVLAENDLTNSYEGLREITGQEHANLSILDTDRFSASKSSDSAVALVEQAEQKNLSLLAARISQDVAKDNISLASSGHLPSLTLDGSYSLSDQSNSSQNYDQDNLNLGLNLVVPLYTGGNTTSLTKQAEYNYVAASEDLEATYRSVVKDVRAFNNNISASIGALRAYEQSVVSAKSALEATEAGFDVGTRTIVDVLDSTRRLYDANKNLSDARYNYILSVLQLRQAVGTLSEQDIVDVNAGLKVASK

SEQ ID NO: 545 - protein, 438 aa, Vibrio mimicus
MKKLLPLFVSAALGTLSSAVWAENLAEIYNQAKDNDPQLLSVAAQRDAAFEAVTSSRSTLLPQINLTAGYNVNRSDQDPRESDLFSAGINFSQELYQRSSWVTLDTAEKKARQADSEYAATQQSLILRVSKAYFEVLRAQDNLEFVRAEKAAVGRQLEQTKQRFEVGLSAITDVHDAQAQYDGVLADEVLAENNLTNSYETLREITGQEYSKLSVLDTKRFAASRTTESTDALIEQAQQKNLSLLSARISQDVARDNISLASSGHLPSLTLDGGYNYGNNSNDSAKGTSGEEYNDFKIGVNLSVPLYSGGNTTSLTKQAEFAYVAASQDLEAAYRSVVKDVRAYNNNINASIGALRAYEQAVISAKSALEATEAGFDVGTRTIVDVLDATRRLYDANKNLSNARYDYILSVLQLRQAIGTLSEQDIMDVNAGLKVAKK

SEQ ID NO: 546 - protein, 442 aa, Vibrio owensii
MKKLLPLFISAALGSMSTSAWADSLAEIYDLAKQNDPQLLSVAAQRDQAFEAINSSRSALLPQINLTAGYDIARGDTDFDSKLRSKQSNDQNVLSAGVGFSQELYNRASWITLDTAEKTARRADATYAATQQGLILRVSQAYFEVLRAQDNLVFVRAEKAAVGRQLEQTKQRFEVGLSAITDVHDAQAQYDAVLADEVLAENDLINSYESLREITGQEHKNLNVLDTARFSASRSSEPAEALIEEAKTTNLSLLSARIAQDIARDNISLASSGHLPTLSLDGGYNYSDTAESTADNTTDGFNVGVNLAVPLYTGGRTTSEVKQAEFGYVSASEDLEAEYRSVVKEVRAQNNNINASIGALKAYEQSVVSAQSALEATEAGFDVGTRTIVDVLDATRRLYDANKNLSDARYNYILAGLQLRQAVGTLSEQDILDVDAGLKPVK

SEQ ID NO: 547 - protein, 448 aa, Vibrio rumoiensis
MKKLLPLIISLTLGGIHLAHADDLAEIYNQAKENDPQLLRSKADRDAAFEAIESSRGNLLPQINLTAGYNINRNINAHNSDPISISHNATEQNAFTAGINVSQELYRRDSWVNLDIAEQNARQQDSAYAAEQQAMILRVSQAYFDVLQAQDNLTFIQAEKKAVGRQLEQTKQRFEVGLSAITDVHDAQAQYDSVLADEILAKNDVTNSYEALREITGQPHKDLSVLDTQRFSANVPRTDQNALVDEAQQKNLDLLTARISQDIAKSNISLAQSGYTPSLTLDGGYQYSDIDEHDDHSATRNYTSNDYNIGVNLVVPLYQGGTTNADVKTAQYQYVSASQQLEATYRNVVKNVRAYYNNINASIGSIRAYEQSVVSAKSALEATEAGYEVGTRTIVDVLDSTRRLYDANRQLSDARYSYVISVLQLKQAVGTLTEQDILDINAGLKRPS

SEQ ID NO: 548 - protein, 451 aa, Vibrio sp.
MKKLLPLIISLALGGVTTAHADDLAQIYNQAKENDPQLLRSKADRDSAFAAIEASRGDLLPQINLTAGYNINRNINAHNSDPLNISSLANEDNAFTAGVNFSQELYRRDSWVNLDIAEQNARQQDSAYAAEQQNLILRTSQAYFGVLQAQDSLTFIRAEKKAVARQLEQTKQRFDVGLSAITDVHDAQAQYDSVLADEIIGENNVTNSYEELREITGQMNKDLALLDTKRFSANSPQTNSNALVDEAQQKNLSLLTARIGQDIAKSNISLANSGYSPSLTLDGGYQYSDIDSHDDHSATGNYTSNDYHIGVNLIVPLYQGGTTSANVKTAEYQYVSASEQLESTYRGVVKNVRAYYNNINASIGTIRAYEQSVISAKSALEATEAGYDVGTRTIVDVLDATRRLYDANSQLSDARYNYILSVLQLKGAVGTLSEQDVLDINAGLIKTAATK

SEQ ID NO: 549 - protein, 441 aa, Vibrio vulnificus
MKKLLPLLIGAALGSLSSSVWADSLAEIYDLAKQNDPQLLSVQAKRDAAFEAVTSSRSTLLPQINLTAGYNLKRGDTDLDAGATIDNDQNALTAGINFSQELYQRSSWITLDNAEKTARQADAAYAATQQGLILRTAQAYFEVLKAQDNLEFVRAEKAAVARQLEQTKQRFEVGLSAITDVHDAQAQYDGVLADEVLAENSLTNSYEALREITGQEHKNLNVLDTKRFSASRSNASAETLIEEAQEKNLSLLSARITKDIAKDNISLASSGHLPSLTLDGGYNYADVSNSAQSDGTTNNFNVGVNLVVPLYTGGNTTSQTKQAEFNYVSASQDLEATYRGVVKEVRAQNNNINASIGALRAYEQSVVSARSALEATEAGFDVGTRTIVDVLDATRRLYDANKNLSNARYNYILSVLQLRQAVGTLSEQDVLDVDAGLIAKK

SEQ ID NO: 550 - protein, 460 aa, Xanthomonas sp.
MIRRSLVLALAAALSPMAAHATDLLQVYEMARNGDPQLAVAESTRLVNREGQVQARAALLPQLDGSAGYTQSHRELEGVDGRSTTKQRQYAIQGSQTIFNWAQFSNLRAQREVAKAADFTLASANNDLITRTSAAYFQVLVGIESLAAAETNEAAAKKQFDYADKRLEVGLAPITDVHEARAQYDQARADTITARNTLKDYYQALTELTGQPVVGLRALPDEFRPEVPAAYSNVDQLVATAIADNPALKAQQLQVSAAEAQVSAARAGHLPTLSLGANVGRSNSWGGQGTVDEAAGNFTTAGRNIDTDSVGITLTIPIFAGGATQSAVRQALSQRDIQQDTYEQQKRALDRNTRNAYQTVVAGISEVEARRLAVVSAQAAYDASQVGLEVGTRTVLDVVQNQRTLFQAQLNYAQSRYNFLQNRLLLGQAIGKLDITDLQDVNRLLSQDAESKLQGSGSLQ

SEQ ID NO: 551 - protein, 460 aa, Xanthomonas sp.
MIRRSLVLALAAALSPMAVHATDLLQVYEMARNGDPQLAVAESTRLVNREGQVQARAALLPQLDGSAGYTQSHRELEGVDGRSTTKQRQYAIQGSQTIFNWAQFSNLRAQREVAKAADFTLASANNDLITRTSAAYFQVLVGIESLAAAETNEAAAKKQFDYADKRLEVGLAPITDVHEARAQYDQARADTITARNTLKDYYQALTELTGQPVVGLRALPDEFRPEVPAAYSNVDQLVATAIADNPALKAQQLQVSAAEAQVSAARAGHLPTLSLGANVGRSNSWGGQGTVDEAAGNFTTAGRNIDTDSVGITLTIPIFAGGATQSAVRQALSQRDIQQDTYEQQKRALDRNTRNAYQTVVAGISEVEARRLAVVSAQAAYDASQVGLEVGTRTVLDVVQNQRTLFQAQLNYAQSRYNFLQNRLLLGQAIGKLDITDLQDVNRLLSQDAESKLQGSGSLQ

SEQ ID NO: 552 - protein, 460 aa, Xanthomonas sp.
MIRRSLVLALAAALSPMAAHATDLLQVYEMARNGDPQLAVAESTRLVNREGQVQARAALLPQLDGSAGYTQSHRELEGVDGRSTTKQRQYAIQGSQTIFNWAQFSNLRAQREVAKAADFTLASANNDLITRTSAAYFQVLVGIESLAAAETNEAAAKKQFDYADKRLEVGLAPITDVHEARAQYDQARADTITARNTLKDYYQALTELTGQPVVGLRALPDEFRPEVPAAYSNVDQLVATAIADNPALKAQQLQVSAAEAQVSAARAGHLPTLSLGANVGRSNSWGGQGTVDEAAGNFTTAGRNIDTDSVGITLTIPIFAGGATQSAVRQALSQRDIQQDTYEQQKRALDRNTRNAYQTVVAGISEVEARRLAVVSAQAAYDASQVGLEVGTRTVLDVVQNQRTLFQAQLNYAQSRYNFLQNRLLLGQAIGKLDITDLQDVNRMLSQDAESKLQGSGSLQ

SEQ ID NO: 553 - protein, 460 aa, Xanthomonas sp.
MIRRSLVLALAAALSPMAAHATDLLQVYEMARNGDPQLAVAESTRLVNREGQVQARAALLPQLDGSASYTQSHRELEGVDGRSTTKQRQYVIQGSQTIFNWAQFSNLRAQREVAKAADFTLASANNDLITRTSAAYFQVLVGIESLAAAETNEAAAKKQFDYADKRLEVGLAPITDVHEARAQYDQARADTITARNTLKDYYQALTELTGQPVVGLRALPDEFRPEVPAAYSNVDQLVATAIADNPALKAQQLQVSAAEAQVSAARAGHLPTLSLGANVGRSNSWGGQGTVDEAAGNFTTAGRDIDTDSVGITLTIPIFAGGATQSAVRQALSQRDIQQDTYEQQKRALDRNTRNAYQTVVAGISEVEARRLAVVSAQAAYDASQVGLEVGTRTVLDVVQNQRTLFQAQLNYAQSRYNFLQNRLLLGQAIGKLDITDLQDVNRLLSQDAESKLQGSGSLQ

SEQ ID NO: 554 - protein, 457 aa, Xanthomonas campestris
MIRRSLVLALAAALSPMAAHATDLLQVYEMARNGDPQLAVAESTRLVNREGQVQARAALLPQLNGAFDYSKAHREIEGQDGRITTSSRSSQIQGSQTIFNWSQFSTLRAQREVAKAADFTLESANNDLITRTSAAYFQVLVGIESLAAAETNEAAAKKQFDYADKRLEVGLAPITDVHEARAQYDQARADTINARNTLKDYYQALTELTGQPVVGLRALPENFRPEVPAAYSNVDQLVASAIADNPALKAQQLQVSAAEASINAARAGHLPTVNLTGSVGRSNSWGTGAAESGVFTTQGRDIDTDSIGISVSIPIFAGGATQSAVRQAISQRDIQQDTYEQQKRALDRNTRNAYQTVVAGISEVEARRLSVVSAQAAYDASQVGLEVGTRTVLDVVQNQRTLFQAQQLYAQARYTFLQNRLLLAQAIGKIDIAELQDVNRLLSVEAEAKLQGSGSLQ

SEQ ID NO: 555 - protein, 459 aa, Xanthomonas oryzae
MIHRSLVLALAAALSPMAANATDLLQVYEMARNGDPQLAVAESTRLVNREGQVQARAALLPQLDGSAGYTQSHRELEGVGRPTTKQRQYAIQGSQTIFNWAQFSNLRAQREVTRAADFTLASANNDLMTRTSAAYFQVLVGIESLAAAETNEAAAKKQFDYADKRLEVGLAPITDVHEARAQYDQARADTINARNTLKDYYQALTELTGQPVVGLRALPDEFRPEVPAAYSNVDQLVATAIADNPALRAQQLQVSAAEAHVSAARAGHLPTLNLSGSMGRTNSWGGGSDVDTAAGNFMTNNGNIDTNSIGITLSIPIFSGGATQSAVRQALSQRDIQQDIYEQQKRALDRNTRNAYQTVVAGISEVEARRLAVVSAQAAYDASQVGLEVGTRTVLDVVQNQRTLFQAQLNYAQSRYTFLQNRLLLGQAIGKLDITDLQDVNRLLSQDAESKLQGSGSLQ

SEQ ID NO: 556 - protein, 459 aa, Xanthomonas oryzae
MIRRSLVLALAAALSPMAANATDLLQVYEMARNGDPQLAVAESTRLVNREGQVQARAALLPQLDGSAGYTQSHRELEGVGRPTTKQRQYAIQGSQTIFNWAQFSNLRAQREVARAADFTLASANNDLMTRTSAAYFQVLVGIESLAAAETNEAAAKKQFDYADKRLEVGLAPITDVHEARAQYDQARADTINARNTLKDYYQALTELTGQPVVGLRALPDAFRPEVPAAYSNVDQLVATAIADNPALRAQQLQVSAAEAQVSAARAGHLPTLNLSGSMGRTNSWGGGSGVDTAAGNFMTNNGNIDTNSIGVTLSIPIFSGGATQSAVRQALSQRDIQQDTYEQQKRALDRNTRNAYQTVVAGISEVEARRLAVVSAQAAYDASQVGLEVGTRTVLDVVQNQRTLFQAQLNYAQSRYTFLQNRLLLGQAIGKLDITDLQDVNRLLSQDAESKLQGSGSLR

SEQ ID NO: 557 - protein, 459 aa, Xanthomonas oryzae
MIHRSLVLALAAALSPMAANATDLLQVYEMARNGDPQLAVAESTRLVNREGQVQARAALLPQLDGSAGYTQSHRELEGVGRPTTKQRQYAIQGSQTIFNWAQFSNLRAQREVTRAADFTLASANNDLMTRTSAAYFQVLVGIESLAAAETNEAAAKKQFDYADKRLEVGLAPITDVHEARAQYDQARADTINARNTLKDYYQALTELTGQPVVGLRALPDEFRPEVPAAYSNVDQLVATAIADNPALRAQQLQVSAAEAHVSAARASHLPTLNLSGSMGRTNSWGGGSDVDTAAGNFMTNNGNIDTNSIGITLSIPIFSGGATQSAVRQALSQRDIQQDIYEQQKRALDRNTRNAYQTVVAGISEVEARRLAVVSAQAAYDASQVGLEVGTRTVLDVVQNQRTLFQAQLNYAQSRYTFLQNRLLLGQAIGKLDITDLQDVNRLLSQDAESKLQGSGSLQ

SEQ ID NO: 558 - protein, 459 aa, Xanthomonas oryzae
MIRRSLVLALAAALSPMAANATDLLQVYEMARNGDPQLAVAESTRLVNREAQVQARAALLPQLDGSAGYTQSHRELEGVGRSTTKQRQYAIQGSQTIFNWAQFSNLRAQREVARAADFTLASANNDLMTRTSAAYFQVLVGIESLAAAETNEAAAKKQFDYADKRLEVGLAPITDVHEARAQYDQARADTINARNTLKDYYQALTELTGQPVVGLRALPDAFRPEVPAAYSNVDQLVATAIADNPALRAQQLQVSAAEAQVSAARAGHLPTLNLSGSMGRTNSWGGGSGVDTAAGNFMTNNGNIDTNSIGVTLSIPIFSGGASQSAVRQALSQRDIQQDTYEQQKRALDRNTRNAYQTVVAGISEVEARRLAVVSAQAAYDASQVGLEVGTRTVLDVVQNQRTLFQAQLNYAQSRYTFLQNRLLLGQAIGKLDITDLQDVNRLLSQDAESKLQGSGSLQ

SEQ ID NO: 559 - protein, 460 aa, Xanthomonas vasicola
MIRRSLVLALAAALSPMAAHATDLLQVYEMARNGDPQLAVAESTRLVNREGQVQARAALLPQLDGSAAYTQSHRELEGIDGRSTTKQRQYAIQGSQTIFNWAQFSNLRAQREVAKAADFTLASANNDLITRTSAAYFQVLVGIESLAAAETNEAAAKKQFDYADKRLEVGLAPITDVHEARAQYDQARADTINARNTLKDYYQALTELTGQPVVGLRALPEEFRPEVPAAYSNVDQLVATAIADNPALKAQQLQVSAAEAQVSAARAGHLPTLNLSGSLGRTNSWGGGTDVDTAAGNFTTNNRNIDTDSIGITLSIPIFAGGATQSAVRQALSQRDIQQDTYEQQKRALDRNTRNAYQTVVAGISEVEARRLAVVSAQAAYDASQVGLEVGTRTVLDVVQNQRTLFQAQLNYAQSRYNFLQNRLLLGQAIGKLDITDLQDVNRLLSQDAESKLQGSGSLQ

SEQ ID NO: 560 - protein, 460 aa, Xanthomonas vasicola
MIRRSLVLALAAALSPMAAHATDLLQVYEMARNGDPQLAVAESTRLVNREGQVQARAALLPQLDGSAAYTQSHRELEGIDGRSTTKQRQYAIQGSQTIFNWAQFSNLRAQREVAKAADFTLASANNDLITRTSAAYFQVLVGIESLAAAETNEAAAKKQFDYADKRLEVGLAPITDVHEARAQYDQARADTINARNTLKDYYQALTELTGQPVVGLRALPEEFRPEVPAAYSNVDQLVATAIADNPALKAQQLQVSAAEAQVSAARAGHLPTLNLSGSLGRTNSWGGGTDVDTAAGNFTTNNRNIDTDSIGITLSIPIFAGGATQLAVRQALSQRDIQQDTYEQQKRALDRNTRNAYQTVVAGISEVEARRLAVVSAQAAYDASQVGLEVGTRTVLDVVQNQRTLFQAQLNYAQSRYNFLQNRLLLGQAIGKLDITDLQDVNRLLSQDAESKLQGSGSLQ

SEQ ID NO: 561 - protein, 460 aa, Xanthomonas vasicola
MIRRSLVLALAAALSPMAAHATDLLQVYEMARNGDPQLAVAESTRLVNREGQVQARAALLPQLDGSAAYTQSHRELEGIDGRSTTKQRQYAIQGSQTIFNWAQFSNLRAQREVAKAADFTLASANNDLITRTSAAYFQVLVGIESLAAAETNEAAAKKQFDYADKRLEVGLAPITDVHEARAQYDQARADTINARNTLKDYYQALTELTGQPVVGLRALPEEFRPEVPAAYSNVDQLVATAIADNPALKAQQLQVSAAEAQVSAARAGHLPTLSLGANVGRSNSWGGQGTVDEAAGNFTTAGRDIDTDSVGITLTIPIFAGGATQSAVRQALSQRDIQQDTYEQQKRALDRNTRNAYQTVVAGISEVEARRLAVVSAQAAYDASQVGLEVGTRTVLDVVQNQRTLFQAQLNYAQSRYNFLQNRLLLGQAIGKLDITDLQDVNRLLSQDAESKLQGSGSLQ

SEQ ID NO: 562 - protein, 452 aa, Xylella fastidiosa
MIRRCLALAVTTALFPVVSQATNLLQVYEMARAADPQLSAAESTRLYSVEGQVQARAALLPQMSGAATLARSRTEYDGISGSAISKNRQYSINGSQTLFNWNQFSNLRSQRAIAKAADFTLESAKQNLIVRSSAAYFSVLIGIESLIAAEANEASAKRQFDYAQKRLEVGLSPITDLHEARASYDQARADTITARNTLQDYYQALAEITGQPVTDLRGLPEDFRPEVPAKFSNIDQLVTEAISNNPSLKAQQLQVNAAESSISAARGEHYPTLSLSGSLGKSKAWGSSSGAMSALIPDRDQPISDTNNLQLTLSIPLFAGGATQSRVRQAIAQRDIQQDNYEQNKRTLNRNTRNAYQNMIAGISEVEARRLAVVSAQAALDASQVGLEVGTRTLLDVVQNQRILFSVRQAYVQARYNFLQNRLLLSQNSGTLSIEDVQEINQLLTADSERKL

SEQ ID NO: 563 - protein, 452 aa, Xylella fastidiosa
MIRRCLALAVTTALFPVVSQATNLLQVYEMARAADPQLSAAESTRLYSVEGQVQARAALLPQMSGTATLARSRTEYDGISGSAISKNRQYSINGSQTLFNWSQFSNLRSQREIAKAADFTLESAKQNLIVRSSAAYFSVLIGIESLIAAEANEASAKRQFDYAQKRLEVGLSPITDLHEARASYDQARADTITARNTLQDYYQALAEITGQPVTDLRGLPEDFRPEVPAKFSNIDQLVTEAISNNPSLKAQQLQVNAAESSISAARGEHYPTLSLSGSLGKSKAWGSSSGAMSALIPDRNQPISDTNNLQLTLSIPLFAGGATQSRVRQAIAQRDIQQDNYEQNKRTLNRNTRNAYQNMIAGISEVEARRLAVVSAQAALDASQVGLEVGTRTLLDVVQNQRILFSVRQAYVQARYNFLQNRLLLSQNSGTLSIEDVQEINQLLTADSERKL

SEQ ID NO: 564 - protein, 452 aa, Xylella fastidiosa
MIRRCLALAVTTALFPVVSQATNLLQVYEMARAADPQLSAAESTRLYSVEGQVQARAALLPQMSGTATLARSRTEYDGISGSAISKTRQYSINGTQTLFNWSQFSNLRSQRAIAKAADFTLESAKQNLIVRSSAAYFSVLIGIESLIAAEANEASAKRQFDYAQKRLEVGLSPITDLHEARASYDQARADTITARNTLQDYYQALTEITGQPVTDLRGLPEDFRPEVPAKFSHIDQLVTEAISNNPSLKAQQLQVNAAESSISAARGEHYPTLSLSGSLGKSKAWGTSSGAMSALIPDRDQPISDTNNLQLTLSIPLFAGGATQSRVRQAIAQRDIQQDTYEQNKRTLNRNTRNAYQNMIAGISEVEARRLAVVSAQAALDASQVGLEVGTRTLLDVVQNQRILFSVRQAYVQARYNFLQNRLLLSQNSGTLSIEDVQEINQLLTADSERKL

SEQ ID NO: 565 - protein, 493 aa, Yersinia sp.
MKKLLPLLIGLSLAGFSTMSQAENLLQVYKQARDSNPDLRKSAADRDAAYEKINQARSPLLPQLGLGAGYTHTNGFRDASDNPDSNATSGSLQLTQTIFDMSKWRALTLQEKAAGIQDVTFQTNEQALILNTATAYFNVLRAIDSLSYTEAQKQSVYRQLDQTTQRFNVGLVAITDVQNARASYDTVLASEVTARNNLDNALENLRQITGVYYPELASLNVSRLKTDRPQAVANLLKEAEKRNLSLLSARLSQDLAREQIKSAETGYMPTIDLTASSSVTNTRYSGGSPTSQQVNNDAGQNQIGVQLSLPLYSGGATNSAVKQAQYNFVGASEQLESAHRSVVQTVRSSFNNISASISSIKAYEQVVISNQSSLDAMEAGYQVGTRTILDVLTATTNLYQSKQQLADARYNYLINQLNIQSALGTLNMDDLMQLNGVLDKPVPTSAASLAPDNANQNAYANGESAQSAPRATSVSQPATTAPAAARSGNPFRN

SEQ ID NO: 566 - protein, 493 aa, Yersinia sp.
MKKLLPLLIGLSLAGFSSMSQAENLLQVYKQARDSNPDLRKSAADRDAAYEKINEARSPLLPQLGLGAGYTHTNGFRDASDNPDSNATSGSLQLTQTIFDMSKWRALTLQEKTAGIQDVTFQTSEQKLILDTATAYFAVLNAIDTLSYTEAQKQSVYRQLDQTTQRFNVGLVAITDVQNARASYDTVLADEVTARNNLDNALENLRLITGVYYPELASLNVDRLKTERPQAVNNLLKDAEKRNLSLLSARLSQDLAREQIKSAETGYMPTIDLTASSSVTNTRYSGGTPSTQQVNTDAGQNKIGVQLSLPLYSGGATNSAVKQAQYNFVGASEQLESAHRSVVQTVRSSFNNISASISSIKAYEQVVISNQSSLDAMEAGYQVGTRTILDVLTATTNLYQSKQQLANARYTYLINQLNIKSALGTLNMNDLMALNGVLDKPVPTSATSLAPDNANQNAYANGTSAQAAPRATAVSQPTTATPAATRNGNPFRN

SEQ ID NO: 567 - protein, 491 aa, Yersinia sp.
MKKLLPLLIGLSLAGFSTMSQAENLLQVYKQARDSNPDLRKSAADRDAAYEKINEARSPLLPQLGLGAGYSHTNGFRDASSNPDSNATSGSLQLTQTIFDMSKWRALTLQEKAAGIQDVTFQTNEQALILNTATAYFNVLRAIDSLSYTEAQKQSVYRQLDQTTQRFNVGLVAITDVQNARASYDTVLADEVTARNNLDNALENLRQITGVYYPELASLNVDRLKTKRPEAVANLLKDAEKRNLSLLSARLSQDLAREQIKSAETGYMPTIDLTASSSVTNTRYSGGTPSTQQVNNDAGQNQIGVQLSLPLYSGGATNSAVKQAQYNFVGASEQLESAHRSVVQTVRSSFNNISASISSIKAYEQVVISNQSSLDAMEAGYQVGTRTILDVLTATTNLYQSKQQLADARYNYLINQLNIQSALGTLNMNDLMELNGVLDKPVPTSATALAPDNANQNAYADGTNTQAAPRVTPVSQPSTTAPARNGNPFRN

SEQ ID NO: 568 - protein, 465 aa, Yersinia pestis
MKKLLPLLIGLSLAGFSTMSQAENLLQVYKQARDSNPDLRKAAADRDAAYEKINEVRSPLLPQLGLSAGYTHANGFRDASNSPDSNATSGSLKLTQTIFDMSKWRALTLQEKAAGIQDVTFQTSEQQLILNTATAYFNVLRAIDSLSYTEAQKQSVYRQLDQTTQRFNVGLVAITDVQNARASYDTVLAAEVAARNNLDNALESLRQITGVYYPELASLNVERLKTQRPDAVNNLLKEAEKRNLSLLSARLSQDLAREQIKSAETGYMPTVDLTASSSITNTRYSGGTPSSQQVNNDSGQNQIGVQFSLPLYSGGATNSAVKQAQYNFVGASELLESAHRNMVQTLRSSFNNISASISSINAYQQVVISNQSSLDAMEAGYQVGTRTILDVLTATTNLYQSKQQLADARYNYLINQLNIKSALGTLNMNDLMALNAVLDKPVPTSAAALAPENTTRQTVTTPRAQ

SEQ ID NO: 569 - protein, 488 aa, Yersinia ruckeri
MKKLLPLLIGLSLAGFSTMSQAENLLQVYKQARESNPDLRKSAADRDAAFEKINESRSPLLPQLGLGADYAYSQGFRDSNGVNSNVTAGSLTLTQTIFDMSKWRALTLQEKTAGIQDVTFQTSEQQLILNTAIAYFNVLRAIDTLSYTEAQKEAVYRQLDQTTQRFNVGLVAITDVQNARATYDSILAQEVTAQNGVDNALENLRQITGVYYPELASLNVDRLKTKRPDAVNNLLSEAEKRNLTLLSARLSQDLAREQIKLAETGYMPTINATASSKISNNRYSGNAVSDQQRSNDAGQNSIGLSFNLPLYSGGATNSQVKQAQYGFVGASEQLESAHRSVVQTVRSSYNNISASISSINAFKQAVISAQSSLDASEAGYQVGTRTILDVLNATTALYNAKQQLANARYDYLINELTIKSSLGTLNQNDLMELNGALDKPISTSATKVTSESSQSASASSYAQTSAQTAPVSQPTATSTRSAGNPFRN

SEQ ID NO: 570 - DNA, 35 bp, synthetic construct
  misc_feature 1..35: note = Synthetic construct
  source 1..35: mol_type = other DNA
tttacggctagctcagtcctaggtattatgctagc

SEQ ID NO: 571 - protein, 16 aa, synthetic construct
  REGION 1..16: note = Synthetic construct
GGGKPIPNPLLGLDST

SEQ ID NO: 572 - protein, 35 aa, synthetic construct
  REGION 1..35: note = Synthetic construct
SFRNGVGSGAKKTSFRRAKQGGKPIPNPLLGLDST

SEQ ID NO: 573 - protein, 24 aa, synthetic construct
  REGION 1..24: note = Synthetic construct
YRWRCKNQGGGKPIPNPLLGLDST

SEQ ID NO: 574 - protein, 26 aa, synthetic construct
  REGION 1..26: note = Synthetic construct
GIGWMLSRARGGGKPIPNPLLGLDST

SEQ ID NO: 575 - protein, 26 aa, synthetic construct
  REGION 1..26: note = Synthetic construct
IVHRYPRICYGGGKPIPNPLLGLDST

SEQ ID NO: 576 - protein, 26 aa, synthetic construct
  REGION 1..26: note = Synthetic construct
SHSMVSVLVRGGGKPIPNPLLGLDST

SEQ ID NO: 577 - protein, 26 aa, synthetic construct
  REGION 1..26: note = Synthetic construct
GIYGHIVVYWGGGKPIPNPLLGLDST

SEQ ID NO: 578 - protein, 26 aa, synthetic construct
  REGION 1..26: note = Synthetic construct
SGLRPWMASVGGGKPIPNPLLGLDST

SEQ ID NO: 579 - protein, 26 aa, synthetic construct
  REGION 1..26: note = Synthetic construct
LSMSICMRPKGGGKPIPNPLLGLDST

SEQ ID NO: 580 - protein, 26 aa, synthetic construct
  REGION 1..26: note = Synthetic construct
VNDRLKLQWVGGGKPIPNPLLGLDST

SEQ ID NO: 581 - protein, 26 aa, synthetic construct
  REGION 1..26: note = Synthetic construct
PGLIDVSYWHGGGKPIPNPLLGLDST

SEQ ID NO: 582 - protein, 26 aa, synthetic construct
  REGION 1..26: note = Synthetic construct
SHSVAPWSLQGGGKPIPNPLLGLDST

SEQ ID NO: 583 - protein, 26 aa, synthetic construct
  REGION 1..26: note = Synthetic construct
RYFTLNNFGWGGGKPIPNPLLGLDST

SEQ ID NO: 584 - protein, 36 aa, synthetic construct
  REGION 1..36: note = Synthetic construct
GSVTRFISFWHMMLLCGMLVGGGKPIPNPLLGLDST

SEQ ID NO: 585 - protein, 36 aa, synthetic construct
  REGION 1..36: note = Synthetic construct
SVIRINCLVLVGRLVLGTQVGGGKPIPNPLLGLDST

SEQ ID NO: 586 - protein, 36 aa, synthetic construct
  REGION 1..36: note = Synthetic construct
GLWRAFMPWWSFFGVVRDSGGGGKPIPNPLLGLDST

SEQ ID NO: 587 - protein, 36 aa, synthetic construct
  REGION 1..36: note = Synthetic construct
ALLRRDVSFRFWHRSVLYVRGGGKPIPNPLLGLDST

SEQ ID NO: 588 - protein, 36 aa, synthetic construct
  REGION 1..36: note = Synthetic construct
NCDRWRGRWVRFILWFGRGKGGGKPIPNPLLGLDST

SEQ ID NO: 589 - protein, 36 aa, synthetic construct
  REGION 1..36: note = Synthetic construct
HNPRRHWMGLITLKLMSCDLGGGKPIPNPLLGLDST

SEQ ID NO: 590 - protein, 36 aa, synthetic construct
  REGION 1..36: note = Synthetic construct
SVCPAFRVDTFRSTGDKYSNGGGKPIPNPLLGLDST

SEQ ID NO: 591 - protein, 36 aa, synthetic construct
  REGION 1..36: note = Synthetic construct
RQRDRCNPWMPIRAKRSLLVGGGKPIPNPLLGLDST

SEQ ID NO: 592 - protein, 36 aa, synthetic construct
  REGION 1..36: note = Synthetic construct
GNARCSMCEIRPLMVWTVSTGGGKPIPNPLLGLDST

SEQ ID NO: 593 - protein, 36 aa, synthetic construct
  REGION 1..36: note = Synthetic construct
GVSYNTMVQRRGGDPARALMGGGKPIPNPLLGLDST

SEQ ID NO: 594 - protein, 66 aa, synthetic construct
  REGION 1..66: note = Synthetic construct
LSNGQCNMHCCPCLEYQDYHKHYSNTESFKQLVWMTHICDNYALSHRAKWGGGKPIPNPLLGLDST

SEQ ID NO: 595 - protein, 66 aa, synthetic construct
  REGION 1..66: note = Synthetic construct
HGVQGINIEKQPKRNNPENEQTRMKMRQERDWSCFAMFHAITRDDSYEQNGGGKPIPNPLLGLDST

SEQ ID NO: 596 - protein, 66 aa, synthetic construct
  REGION 1..66: note = Synthetic construct
ETCHWMELHIPLFETDSFKPYDPKSLDSGHCLYYGFFFKYIGGLHAMCMYGGGKPIPNPLLGLDST

SEQ ID NO: 597 - protein, 66 aa, synthetic construct
  REGION 1..66: note = Synthetic construct
VQMIWGFCSGWPMITWYAMMFAHIQWAFWNTKISSRGWEFMASAWPEYFVGGGKPIPNPLLGLDST

SEQ ID NO: 598 - protein, 66 aa, synthetic construct
  REGION 1..66: note = Synthetic construct
IMECSTLICTTLDCFICVQGQWRYCMWNQCWCVCMNWTKTNCAQYSVAKHGGGKPIPNPLLGLDST

SEQ ID NO: 599 - protein, 66 aa, synthetic construct
  REGION 1..66: note = Synthetic construct
IICWWPHPQCCWNFEYCFRKNYLTCFQCSEQYSTTFAPVNAFPTIWQIIYGGGKPIPNPLLGLDST

SEQ ID NO: 600 - protein, 66 aa, synthetic construct
  REGION 1..66: note = Synthetic construct
GSVIVLDWVQGTNLVHKQHNTINRRHHHSHQEMYPWPTVFEMHGNRIIEEGGGKPIPNPLLGLDST

SEQ ID NO: 601 - protein, 66 aa, synthetic construct
  REGION 1..66: note = Synthetic construct
HVPKWWYKGFDWTTQVWPYAAMLGFINAHHLDTVMIKIHLGAFHNCDWVEGGGKPIPNPLLGLDST

SEQ ID NO: 602 - protein, 66 aa, synthetic construct
  REGION 1..66: note = Synthetic construct
VTTFKLWAKALVAFMYDADHHVNDFLPTLYRVYTTMNIWHFKHKCPCTSYGGGKPIPNPLLGLDST

SEQ ID NO: 603 - protein, 66 aa, synthetic construct
  REGION 1..66: note = Synthetic construct
DDDDELLRLCDNITFFMMCIHEFTMKPWFKTIWFLMCWNAFLNQGSNSTHGGGKPIPNPLLGLDST

SEQ ID NO: 604 - protein, 116 aa, synthetic construct
  REGION 1..116: note = Synthetic construct
YMCWHQAINVMEPCAQFYQDIVLSSRVQWQDMDMSMPRLYKMQYVAKSHFSIMYFIHREDIQSSGCCDCNCNPVRKIFCTRYVEMVDCGYWRHFWLPPEEGGGKPIPNPLLGLDST

SEQ ID NO: 605 - protein, 116 aa, synthetic construct
  REGION 1..116: note = Synthetic construct
FIIYIPKVYDWYAASMGCTYPDGLGFRMVSMRVIWYAWYVYSCYTAVETNKFGDCGCGNYNHPDQEQIRYKTSEQESTKMFFRWAPEWQNNHRHHLMVEMGGGKPIPNPLLGLDST

SEQ ID NO: 606 - protein, 116 aa, synthetic construct
  REGION 1..116: note = Synthetic construct
HSYCEWLMAKILSMGEQWWHKYYFGLRHQFNVSKGYHFNTSFDFHRCGEPKNNYYARTHCEKEYSNDDVHPRQNSMGRWGAEWPALIFKPLFGINKWGNFGGGKPIPNPLLGLDST

SEQ ID NO: 607 - protein, 116 aa, synthetic construct
  REGION 1..116: note = Synthetic construct
SGIYCQITRWVHPFSESTQNMDNMANTKRKPQWHYPRRHQHKEAQFVFFGIARGWPFQFFEQGWVTIDVHEEHLWIFCFGERNMEHGNVERAPTISSRIKGGGKPIPNPLLGLDST

SEQ ID NO: 608 - protein, 116 aa, synthetic construct
  REGION 1..116: note = Synthetic construct
KHWKQRHVYYRQAVYQQQKCQMYNVPYSPASGCFNCQPNCHRKDFDWRDHTGYCFKMLQFHNFITPKRCTESALFAGQECRPDFAQESGAQKDFSGMHPGGGGKPIPNPLLGLDST

SEQ ID NO: 609 - protein, 116 aa, synthetic construct
  REGION 1..116: note = Synthetic construct
FNPESSHQPSTIPKSHFRIICHFVRDWHFPCGSWTFSVVDIYFCEMYTTLGNPHGFVICCTYGSQYSGDNRCADKLERHPAMMENTYGWHGHTSAGLAQPGGGKPIPNPLLGLDST

SEQ ID NO: 610 - protein, 116 aa, synthetic construct
  REGION 1..116: note = Synthetic construct
DKMEPQWNHSPRCFASLCCGGSHTMMAWNHVISWKGRDLVIGVNRHCATPPHSQFWHNAWWQGFIKHVIETPRLGNMAKNMQCFAACALAVAFPVDISQLGGGKPIPNPLLGLDST

SEQ ID NO: 611 - protein, 116 aa, synthetic construct
  REGION 1..116: note = Synthetic construct
RGKWMMCNTHMAHWNTYFWDGSAVHLTDDFYRNGPAKSYNLFMVQNHKESRHNYKVCFFCYLITTYITIAKHKRMNENYWWMAQVYLKFVRWHARNCCYAGGGKPIPNPLLGLDST

SEQ ID NO: 612 - protein, 116 aa, synthetic construct
  REGION 1..116: note = Synthetic construct
VMQGDTWWECEPSEAEIQMLYWPWGSQKDPIDWAYLCDTWKYTGDLCSGGPEQPDEHRIHDAIGRAFYRPCPSLNMYYLSQRWAIFDTHNSLAAGSYCFMGGGKPIPNPLLGLDST

SEQ ID NO: 613 - protein, 116 aa, synthetic construct
  REGION 1..116: note = Synthetic construct
TGRQVTEVTVWHALTTICGISELEFTYGACPMWENMELEKFSGNVCYELQDHCFCDWWQYTERCLENLPMIELPIQWKPFTLHEWWIIGRCPLTIMNSWAGGGKPIPNPLLGLDST

SEQ ID NO: 614 - DNA, 200 bp, synthetic construct
  misc_feature 1..200: note = Synthetic construct
  misc_feature 72..73: note = n is a, c, g, or t
  misc_feature 75..76: note = n is a, c, g, or t
  misc_feature 78..79: note = n is a, c, g, or t
  misc_feature 81..82: note = n is a, c, g, or t
  misc_feature 84..85: note = n is a, c, g, or t
  misc_feature 87..88: note = n is a, c, g, or t
  misc_feature 90..91: note = n is a, c, g, or t
  misc_feature 93..94: note = n is a, c, g, or t
  misc_feature 96..97: note = n is a, c, g, or t
  misc_feature 99..100: note = n is a, c, g, or t
  misc_feature 102..103: note = n is a, c, g, or t
  misc_feature 105..106: note = n is a, c, g, or t
  misc_feature 108..109: note = n is a, c, g, or t
  misc_feature 111..112: note = n is a, c, g, or t
  misc_feature 114..115: note = n is a, c, g, or t
  misc_feature 117..118: note = n is a, c, g, or t
  misc_feature 120..121: note = n is a, c, g, or t
  misc_feature 123..124: note = n is a, c, g, or t
  misc_feature 126..127: note = n is a, c, g, or t
  misc_feature 129..130: note = n is a, c, g, or t
  source 1..200: mol_type = other DNA
atagagctcgaattcaggaggaaacgatgagaactctgactctaaatgaattagattctgtttctggtggtnnknnknnknnknnknnknnknnknnknnknnknnknnknnknnknnknnknnknnknnkggaggaggtaaacctattcctaatcctctcctaggtttagattctacttaagtcgacaggaggaaacga

SEQ ID NO: 615 - DNA, 48 bp, synthetic construct
  misc_feature 1..48: note = Synthetic construct
  source 1..48: mol_type = other DNA
atagagctcgaattcaggaggaaacgatgagaactctgactctaaatg

SEQ ID NO: 616 - DNA, 36 bp, synthetic construct
  misc_feature 1..36: note = Synthetic construct
  source 1..36: mol_type = other DNA
tatgtcgacttaagtagaatctaaacctaggagagg

SEQ ID NO: 617 - DNA, 30 bp, synthetic construct
  misc_feature 1..30: note = Synthetic construct
  source 1..30: mol_type = other DNA
catcagacggagaccgctgaatctggactg

SEQ ID NO: 618 - DNA, 18 bp, synthetic construct
  misc_feature 1..18: note = Synthetic construct
  source 1..18: mol_type = other DNA
gagcccgggtggccatcc

SEQ ID NO: 619 - DNA, 32 bp, synthetic construct
  misc_feature 1..32: note = Synthetic construct
  source 1..32: mol_type = other DNA
gtctgcttacataaacagtaatacaaggggtg

SEQ ID NO: 620 - DNA, 39 bp, synthetic construct
  misc_feature 1..39: note = Synthetic construct
  source 1..39: mol_type = other DNA
gcgccacaggtgcggttgctatctccttgctgcctcgcg

SEQ ID NO: 621 - DNA, 37 bp, synthetic construct
  misc_feature 1..37: note = Synthetic construct
  source 1..37: mol_type = other DNA
gcgcgaggcagcaaggagatagcaaccgcacctgtgg

SEQ ID NO: 622 - DNA, 57 bp, synthetic construct
  misc_feature 1..57: note = Synthetic construct
  source 1..57: mol_type = other DNA
gctcataacaccccttgtattactgtttatgtaagcagacagttttattgttcatga

SEQ ID NO: 623 - DNA, 304 bp, synthetic construct
  misc_feature 1..304: note = Synthetic construct
  source 1..304: mol_type = other DNA
atagagctcgaattcaggaggaaacgatgagaactctgactctaaatgaattagattctgtttctggcgccggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggactagtggtctccccatgcata

SEQ ID NO: 624 - DNA, 29 bp, synthetic construct
  misc_feature 1..29: note = Synthetic construct
  source 1..29: mol_type = other DNA
tatgcatggggagaccactagtccacact

SEQ ID NO: 625 - DNA, 337 bp, synthetic construct
  misc_feature 1..337: note = Synthetic construct
  source 1..337: mol_type = other DNA
gagctcgaattcaggaggaaacgatgagaactctgactctaaatgaattagattctgtttctggtggtaccgaatttggcagcgaactgaaaagctttccggaagtggtgggcaaaaccgtggatcaggcgcgcgaatattttaccctgcattatccgcagtatgatgtgtattttctgccggaaggcagcccggtgaccctggatctgcgctataaccgcgtgcgcgtgttttataacccgggcaccaacgtggtgaaccatgtgccgcatgtgggcggaggaggtaaacctattcctaatcctctcctaggtttagattctacttaaaagtcgac

SEQ ID NO: 626 - DNA, 32 bp, synthetic construct
  misc_feature 1..32: note = Synthetic construct
  source 1..32: mol_type = other DNA
atagtcgacttagcccacatgcggcacatggt

SEQ ID NO: 627 - DNA, 74 bp, synthetic construct
  misc_feature 1..74: note = Synthetic construct
  misc_feature 9..10: note = n is a, c, g, or t
  misc_feature 12..13: note = n is a, c, g, or t
  misc_feature 15..16: note = n is a, c, g, or t
  misc_feature 18..19: note = n is a, c, g, or t
  misc_feature 21..22: note = n is a, c, g, or t
  misc_feature 24..25: note = n is a, c, g, or t
  misc_feature 27..28: note = n is a, c, g, or t
  misc_feature 30..31: note = n is a, c, g, or t
  misc_feature 33..34: note = n is a, c, g, or t
  misc_feature 36..37: note = n is a, c, g, or t
  source 1..74: mol_type = other DNA
atcccgggnnknnknnknnknnknnknnknnknnknnkggaggaggtaaacctattcctaatcctctcctaggt

SEQ ID NO: 628 - DNA, 30 bp, synthetic construct
  misc_feature 1..30: note = Synthetic construct
  source 1..30: mol_type = other DNA
atagagctcgaagaccatggcgtgcagggc

SEQ ID NO: 629 - DNA, 41 bp, synthetic construct
  misc_feature 1..41: note = Synthetic construct
  source 1..41: mol_type = other DNA
atgaagacaagccatggttctgttcatagctatcatcgcgg

SEQ ID NO: 630 - DNA, 132 bp, synthetic construct
  misc_feature 1..132: note = Synthetic construct
  source 1..132: mol_type = other DNA
aaatactacggcaatggggtgacctgtgggaaacattcctgctccgttgactgggggaaagcgaccacctgtatcatcaataacggagcgatggcctgggctacgggcggtcaccagggcaatcacaagtgt

SEQ ID NO: 631 - DNA, 89 bp, synthetic construct
  misc_feature 1..89: note = Synthetic construct
  source 1..89: mol_type = other DNA
atagagctcaggaggaaacgatgagaactctgactctaaatgaattagattctgtttctggtggtaaatactacggcaatggtgtaacg

SEQ ID NO: 632 - DNA, 35 bp, synthetic construct
  misc_feature 1..35: note = Synthetic construct
  source 1..35: mol_type = other DNA
atatctagattagcacttatgatttccctggtggc

SEQ ID NO: 633 - DNA, 72 bp, synthetic construct
  misc_feature 1..72: note = Synthetic construct
  source 1..72: mol_type = other DNA
atcccgggtggcactggctgcagctgaaaccgggtcagccgatgtactaaggtacctggggatcctctagag

SEQ ID NO: 634 - DNA, 180 bp, synthetic construct
  misc_feature 1..180: note = Synthetic construct
  source 1..180: mol_type = other DNA
atacccgggaacagcgatagcgaatgcccgctgagccatgatggctattgcctgcatgatggcgtgtgcatgtatattgaagcgctggataaatatgcgtgcaactgcgtggtgggctatattggcgaacgctgccagtatcgcgatctgaaatggtgggaactgcgctaagtcgacata

SEQ ID NO: 635 - DNA, 20 bp, synthetic construct
  misc_feature 1..20: note = Synthetic construct
  source 1..20: mol_type = other DNA
atacccgggaacagcgatag

SEQ ID NO: 636 - DNA, 23 bp, synthetic construct
  misc_feature 1..23: note = Synthetic construct
  source 1..23: mol_type = other DNA
tatgtcgacttagcgcagttccc

SEQ ID NO: 637 - DNA, 517 bp, synthetic construct
  misc_feature 1..517: note = Synthetic construct
  source 1..517: mol_type = other DNA
acactttgctatgcccgggtatatgtgctggcatcaggcgattaacgtgatggaaccgtgcgcgcagttttatcaggatattgtgctgagcagccgcgtgcagtggcaggatatggatatgagcatgccgcgcctgtataaaatgcagtatgtggcgaaaagccattttagcattatgtattttattcatcgcgaagatattcagagcagcggctgctgcgattgcaactgcaacccggtgcgcaaaattttttgcacccgctatgtggaaatggtggattgcggctattggcgccatttttggctgccgccggaagaaggaggaggtaaacctattcctaatcccgggctgagcaacggccagtgcaacatgcattgctgcccgtgcctggaatatcaggattatcataaacattatagcaacaccgaaagctttaaacagctggtgtggatgacccatatttgcgataactatgcgctgagccatcgcgcgaaatggggaggacgtgatgtcttc

SEQ ID NO: 638 - DNA, 499 bp, synthetic construct
  misc_feature 1..499: note = Synthetic construct
  source 1..499: mol_type = other DNA
acactttgctatgcccgggtttattatttatattccgaaagtgtatgattggtatgcggcgagcatgggctgcacctatccggatggcctgggctttcgcatggtgagcatgcgcgtgatttggtatgcgtggtatgtgtatagctgctataccgcggtggaaaccaacaaatttggcgattgcggctgcggcaactataaccatccggatcaggaacagattcgctataaaaccagcgaacaggaaagcaccaaaatgttttttcgctgggcgccggaatggcagaacaaccacaggcaccacctgatggtggaaatgggaggaggtaaacctattcctaatcccgggcatggcgtgcagggcattaacattgaaaaacagccgaaacgcaacaacccggaaaacgaacagacccgcatgaaaatgcgccaggaacgcgattggagctgctttgcgatgtttcatgcgattacccgcgatgatagctatgaacagaac

SEQ ID NO: 639 - DNA, 517 bp, synthetic construct
  misc_feature 1..517: note = Synthetic construct
  source 1..517: mol_type = other DNA
acactttgctatgcccgggcacagttattgcgaatggctgatggcgaaaattctgagcatgggcgaacagtggtggcataaatattattttggcctgcgccatcagtttaacgtgagcaaaggctatcattttaacaccagctttgattttcatcgctgcggcgaaccgaaaaacaactattatgcgcgcacccattgcgaaaaagaatatagcaacgatgatgtgcatccgcgccagaacagcatgggccgctggggcgcggaatggccggcgctgatttttaaaccgctgtttggcattaacaaatggggcaactttggaggaggtaaacctattcctaatcccggggaaacctgccattggatggaactgcatattccgctgtttgaaaccgatagctttaaaccgtatgatccgaaaagcctggatagcggccattgcctgtattatggctttttttttaaatatattggcggcctgcatgcgatgtgcatgtatggaggacgtgatgtcttc

SEQ ID NO: 640 - DNA, 517 bp, synthetic construct
  misc_feature 1..517: note = Synthetic construct
  source 1..517: mol_type = other DNA
acactttgctatgcccgggagcggcatttattgccagattacccgctgggtgcatccgtttagcgaaagcacccagaacatggataacatggcgaacaccaaacgcaaaccgcagtggcattatccgcgccgccatcagcataaagaagcgcagtttgtgttttttggcattgcgcgcggctggccgtttcagttttttgaacagggctgggtgaccattgatgtgcatgaagaacatctgtggattttttgctttggcgaacgcaacatggaacatggcaacgtggaacgcgcgccgaccattagcagccgcattaaaggaggaggtaaacctattcctaatcccggggtgcagatgatttggggcttttgcagcggctggccgatgattacctggtatgcgatgatgtttgcgcatattcagtgggcgttttggaacaccaaaattagcagccgcggctgggaatttatggcgagcgcgtggccggaatattttgtgggaggacgtgatgtcttc

SEQ ID NO: 641 - DNA, 517 bp, synthetic construct
  misc_feature 1..517: note = Synthetic construct
  source 1..517: mol_type = other DNA
acactttgctatgcccgggaaacattggaaacagcgccatgtgtattatcgccaggcggtgtatcagcagcagaaatgccagatgtataacgtgccgtatagcccggcgagcggctgctttaactgccagccgaactgccatcgcaaagattttgattggcgcgatcataccggctattgctttaaaatgctgcagtttcataactttattaccccgaaacgctgcaccgaaagcgcgctgtttgcgggccaggaatgccgcccggattttgcgcaggaaagcggcgcgcagaaagattttagcggcatgcatccgggcggaggaggtaaacctattcctaatcccgggattatggaatgcagcaccctgatttgcaccaccctggattgctttatttgcgtgcagggccagtggcgctattgcatgtggaaccagtgctggtgcgtgtgcatgaactggaccaaaaccaactgcgcgcagtatagcgtggcgaaacatggaggacgtgatgtcttc

SEQ ID NO: 642 - DNA, 517 bp, synthetic construct
  misc_feature 1..517: note = Synthetic construct
  source 1..517: mol_type = other DNA
acactttgctatgcccgggtttaacccggaaagcagccatcagccgagcaccattccgaaaagccattttcgcattatttgccattttgtgcgcgattggcattttccgtgcggcagctggacctttagcgtggtggatatttatttttgcgaaatgtataccaccctgggcaacccgcatggctttgtgatttgctgcacctatggcagccagtatagcggcgataaccgctgcgcggataaactggaacgccatccggcgatgatggaaaacacctatggctggcatggccataccagcgcgggcctggcgcagccgggaggaggtaaacctattcctaatcccgggattatttgctggtggccgcatccgcagtgctgctggaactttgaatattgctttcgcaaaaactatctgacctgctttcagtgcagcgaacagtatagcaccacctttgcgccggtgaacgcgtttccgaccatttggcagattatttatggaggacgtgatgtcttc

SEQ ID NO: 643 - DNA, 517 bp, synthetic construct
  misc_feature 1..517: note = Synthetic construct
  source 1..517: mol_type = other DNA
acactttgctatgcccggggataaaatggaaccgcagtggaaccatagcccgcgctgctttgcgagcctgtgctgcggcggcagccataccatgatggcgtggaaccatgtgattagctggaaaggccgcgatctggtgattggcgtgaaccgccattgcgcgaccccgccgcatagccagttttggcataacgcgtggtggcagggctttattaaacatgtgattgaaaccccgcgcctgggcaacatggcgaaaaacatgcagtgctttgcggcgtgcgcgctggcggtggcgtttccggtggatattagccagctgggaggaggtaaacctattcctaatcccgggggcagcgtgattgtgctggattgggtgcagggcaccaacctggtgcataaacagcataacaccattaaccgccgccatcatcatagccatcaggaaatgtatccgtggccgaccgtgtttgaaatgcatgggaaccgcattattgaagaaggaggacgtgatgtcttc

SEQ ID NO: 644 - DNA, 517 bp, synthetic construct
  misc_feature 1..517: note = Synthetic construct
  source 1..517: mol_type = other DNA
acactttgctatgcccgggcgcggcaaatggatgatgtgcaacacccatatggcgcattggaacacctatttttgggatggcagcgcggtgcatctgaccgatgatttttatcgcaacggcccggcgaaaagctataacctgtttatggtgcagaaccataaagaaagccgccataactataaagtgtgctttttttgctatctgattaccacctatattaccattgcgaaacataaacgcatgaacgaaaactattggtggatggcgcaggtgtatctgaaatttgtgcgctggcatgcgcgcaactgctgctatgcgggaggaggtaaacctattcctaatcccgggcatgtgccgaaatggtggtataaaggctttgattggaccacccaggtgtggccgtatgcggcgatgctgggctttattaacgcgcatcatctggataccgtgatgattaaaattcatctgggcgcgtttcataactgcgattgggtggaaggaggacgtgatgtcttc

SEQ ID NO: 645 - DNA, 517 bp, synthetic construct
  misc_feature 1..517: note = Synthetic construct
  source 1..517: mol_type = other DNA
acactttgctatgcccggggtgatgcagggcgatacctggtgggaatgcgaaccgagcgaagcggaaattcagatgctgtattggccgtggggcagccagaaagatccgattgattgggcgtatctgtgcgatacctggaaatataccggcgatctgtgcagcggcggcccggaacagccggatgaacatcgcattcatgatgcgattggccgcgcgttttatcgcccgtgcccgagcctgaacatgtattatctgagccagcgctgggcgatttttgatacccataacagcctggcggcgggcagctattgctttatgggaggaggtaaacctattcctaatcccggggtgaccacctttaaactgtgggcgaaagcgctggtggcgtttatgtatgatgcggatcatcatgtgaacgattttctgccgaccctgtatcgcgtgtataccaccatgaacatttggcattttaaacataaatgcccgtgcaccagctatggaggacgtgatgtcttc

SEQ ID NO: 646 - DNA, 517 bp, synthetic construct
  misc_feature 1..517: note = Synthetic construct
  source 1..517: mol_type = other DNA
acactttgctatgcccgggaccggccgccaggtgaccgaagtgaccgtgtggcatgcgctgaccaccatttgcggcattagcgaactggaatttacctatggcgcgtgcccgatgtgggaaaacatggaactggaaaaatttagcggcaacgtgtgctatgaactgcaggatcattgcttttgcgattggtggcagtataccgaacgctgcctggaaaacctgccgatgattgaactgccgattcagtggaaaccgtttaccctgcatgaatggtggattattggccgctgcccgctgaccattatgaacagctgggcgggaggaggtaaacctattcctaatcccggggatgatgatgatgaactgctgcgcctgtgcgataacattaccttttttatgatgtgcattcatgaatttaccatgaaaccgtggtttaaaaccatttggtttctgatgtgctggaacgcgtttctgaaccagggcagcaatagcacccatggaggacgtgatgtcttc

SEQ ID NO: 647 - DNA, 19 bp, synthetic construct
  misc_feature 1..19: note = Synthetic construct
  source 1..19: mol_type = other DNA
acactttgctatgcccggg

SEQ ID NO: 648 - DNA, 27 bp, synthetic construct
  misc_feature 1..27: note = Synthetic construct
  source 1..27: mol_type = other DNA
gggattaggaataggtttacctcctcc

SEQ ID NO: 649 - DNA, 26 bp, synthetic construct
  misc_feature 1..26: note = Synthetic construct
  source 1..26: mol_type = other DNA
gaggtaaacctattcctaatcccggg

SEQ ID NO: 650 - DNA, 38 bp, synthetic construct
  misc_feature 1..38: note = Synthetic construct
  source 1..38: mol_type = other DNA
aggaataggtttacctcctccccatttcgcgcgatggc

SEQ ID NO: 651 - DNA, 46 bp, synthetic construct
  misc_feature 1..46: note = Synthetic construct
  source 1..46: mol_type = other DNA
aggaataggtttacctcctccgttctgttcatagctatcatcgcgg

SEQ ID NO: 652 - DNA, 42 bp, synthetic construct
  misc_feature 1..42: note = Synthetic construct
  source 1..42: mol_type = other DNA
aggaataggtttacctcctccatacatgcacatcgcatgcag

SEQ ID NO: 653 - DNA, 42 bp, synthetic construct
  misc_feature 1..42: note = Synthetic construct
  source 1..42: mol_type = other DNA
aggaataggtttacctcctcccacaaaatattccggccacgc

SEQ ID NO: 654 - DNA, 43 bp, synthetic construct
  misc_feature 1..43: note = Synthetic construct
  source 1..43: mol_type = other DNA
aggaataggtttacctcctccatgtttcgccacgctatactgc

SEQ ID NO: 655 - DNA, 50 bp, synthetic construct
  misc_feature 1..50: note = Synthetic construct
  source 1..50: mol_type = other DNA
aggaataggtttacctcctccataaataatctgccaaatggtcggaaacg

SEQ ID NO: 656 - DNA, 46 bp, synthetic construct
  misc_feature 1..46: note = Synthetic construct
  source 1..46: mol_type = other DNA
aggaataggtttacctcctccttcttcaataatgcggttcccatgc

SEQ ID NO: 657 - DNA, 45 bp, synthetic construct
  misc_feature 1..45: note = Synthetic construct
  source 1..45: mol_type = other DNA
aggaataggtttacctcctccttccacccaatcgcagttatgaaa

SEQ ID NO: 658 - DNA, 40 bp, synthetic construct
  misc_feature 1..40: note = Synthetic construct
  source 1..40: mol_type = other DNA
aggaataggtttacctcctccatagctggtgcacgggcat

SEQ ID NO: 659 - DNA, 41 bp, synthetic construct
  misc_feature 1..41: note = Synthetic construct
  source 1..41: mol_type = other DNA
aggaataggtttacctcctccatgggtgctattgctgccct

SEQ ID NO: 660 - DNA, 60 bp, synthetic construct
  misc_feature 1..60: note = Synthetic construct
  source 1..60: mol_type = other DNA
atcccgggctgtctatgtctatctgcatgcgtccgaaaggaggaggtaaacctattccta

SEQ ID NO: 661 - DNA, 90 bp, synthetic construct
  misc_feature 1..90: note = Synthetic construct
  source 1..90: mol_type = other DNA
atcccgggctgtctatgtctatctgcatgcgtccgaaactgtctatgtctatctgcatgcgtccgaaaggaggaggtaaacctattccta

SEQ ID NO: 662 - DNA, 55 bp, synthetic construct
  misc_feature 1..55: note = Synthetic construct
  source 1..55: mol_type = other DNA
ggtaccaggaggaaacgatggatagaaaaagaacaaaattagagttgttatttgc

SEQ ID NO: 663 - DNA, 34 bp, synthetic construct
  misc_feature 1..34: note = Synthetic construct
  source 1..34: mol_type = other DNA
ggtggtgaattcaggaggaaacgatggatagaaa

SEQ ID NO: 664 - DNA, 29 bp, synthetic construct
  misc_feature 1..29: note = Synthetic construct
  source 1..29: mol_type = other DNA
gcgtggtacctcatttagagtcagagttc

SEQ ID NO: 665 - DNA, 33 bp, synthetic construct
  misc_feature 1..33: note = Synthetic construct
  source 1..33: mol_type = other DNA
ggtggtggtaccatgagaactctgactctaaat

SEQ ID NO: 666 - DNA, 36 bp, synthetic construct
  misc_feature 1..36: note = Synthetic construct
  source 1..36: mol_type = other DNA
ggcggcgtcgactctagattataaacaaacatcact

SEQ ID NO: 667 - DNA, 32 bp, synthetic construct
  misc_feature 1..32: note = Synthetic construct
  source 1..32: mol_type = other DNA
atgaggtaccatggcttcagggcgtgatattg

SEQ ID NO: 668 - DNA, 90 bp, synthetic construct
  misc_feature 1..90: note = Synthetic construct
  source 1..90: mol_type = other DNA
caggtcgacttaagtagaatctaaacctaggagaggattaggaataggtttacctcctcctaaacaaacatcactaagattatttggact

SEQ ID NO: 669 - DNA, 197 bp, synthetic construct
  misc_feature 1..197: note = Synthetic construct
  source 1..197: mol_type = other DNA
atatctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtaggaggaaacgatgtttcgccaggatgctttagaaaac

SEQ ID NO: 670 - DNA, 52 bp, synthetic construct
  misc_feature 1..52: note = Synthetic construct
  source 1..52: mol_type = other DNA
atacctgagggtattatttaatataagaaagaacagttattggacaatccac

SEQ ID NO: 671 - DNA, 52 bp, synthetic construct
  misc_feature 1..52: note = Synthetic construct
  source 1..52: mol_type = other DNA
atagagctctgggtacccggggatcctctagagtcgacaggaggaaacgatg

SEQ ID NO: 672 - DNA, 32 bp, synthetic construct
  misc_feature 1..32: note = Synthetic construct
  source 1..32: mol_type = other DNA
atagcatgcctgcagttaaatagaaataactc

SEQ ID NO: 673 - protein, 15 aa, synthetic construct
MKELNLIEVEQVSGA

SEQ ID NO: 674 - protein, 15 aa, synthetic construct
MKELNKVEVEQVSGA

SEQ ID NO: 675 - protein, 15 aa, synthetic construct
MRELTSVEMQNVSGA

SEQ ID NO: 676 - protein, 15 aa, synthetic construct
MRELKTNEIDGVSGG

SEQ ID NO: 677 - protein, 15 aa, synthetic construct
MRELTSYELQAVSGG

SEQ ID NO: 678 - protein, 15 aa, synthetic construct
MRELNVMEVEAVSGA

SEQ ID NO: 679 - protein, 714 aa, synthetic construct
MIFRKVLQDQSMNRNQFQHILQSLHFGWKRKIPQILQTEAAECGLACLTMICRYYGMNVDLFSLRNQIGVSSHGVTLATLINMSTSVHLISRPLSLDLNEIKQLKTPCILHWDLNHFVVLVAVKRSKFILHDPAFGRREVGLNEMSKHFSGIALELWPDKEFKPRTERSQLSLIKMLFQIDGLKGFLVKILSLSLIIESINLLLPVGTQLVMDHVIMAKDHSLLGLICVGLLFFILFRTFIAMLRSWSSLIMGSLVDIQWKAGLFGHLMKLPLAYFEKRKLGDIQSRFSSLETICKTLTNNIVKSIINILMSIGVFIMMFLYGGWLVWIVCGFTLIYIILRVATYQRYRQASEEKIVKEAKANSHFMESLYGIDTLKSLKLTESRAQYWLNMNIDTTNANIRLTKLEMIFSGVNTLIMMIEQTSILWIGAALVMDDKMTLGMFIAFTAYRAQFSEQAANLINMVLDLKMLNLHTERLTDIVLTDTENDIESKYSFPKDIPVEFELRDIAYQYDNLSKPIFTNINMKIQAGESVAIIGPSGAGKTTLMKVMNGLLTPTHGEVLINGIDIYKFGVNNYRDIIGSVLQNDKLFAGSISDNISSFDTEKNYEWLVECAKYCNIHNEIMSMPMGYETLVSELGNSLSGGQKQRLLIARALYRKPCLLFLDEATSHLDEDNEFTINKAISSLKITRIIIAHRKSTIESADRIVEIGSVNH

SEQ ID NO: 680 - protein, 714 aa, synthetic construct
MIFRKVLQDQSMARNQFQHILQSLHFGWRRKIPQILQTEAAECGLACLTMICRYYGMNVDLFSLRNQIGVSSHGVTLATLINMSACVQLQSRPLSLDLNEINQLKTPCILHWDLNHFVVLVAVKRNKFILHDPAFGRREVGINEMSKHFSGIALELWPDKEFQPRTQRSQLSLLKMLFQIDGLKGFLVKILSLSLIIESINLLLPIGTQLVMDHVIIAKDHSLLGLICIGLLFFILFRTFIAMLRSWSSLVMESLVDIQWKAGLFGHLMKLPLAYFEKRKLGDIQSRFVSLEVIRSTLTKSIVSSIINILMTIGVFIMMLLYGGWLVWIVCGFTLIYIILRVVTYQRYRQASEEKIVKEAKANSHFMESLYGIDTLKSLKLTESRAQYWLNMNIDTTNANIRLTKLDMIFNGVNTLIMLVEQTLILWVGASLVMNDKMTLGMFIAFTAYRSQFSEQAANLINMVLDLKMLNLHTERLTDIVLTNTENDIESKNQLPKDIPVEFELNNIAYQYDNLSQPIFSNINMKIKAGESVAIIGPSGAGKTTLMKVMNGLLEPTQGEVLINGIDIYKFGVNNYRDIVGSVLQNDKLFAGSISDNIASFDNEKNHEWLVECAKHCNIHNEIMKMPMGYETLVSELGNSLSGGQKQRLLIARALYRKPCLLFLDEATSHLDEDNEAKINNAISSLKITRIIIAHRTSTIASADRVIDITSVNH

SEQ ID NO: 681 - protein, 727 aa, synthetic construct
MLLLEKLNFKWFNRLPMIRQSQAAECGLACLGMIANYHGHQIDMITLRRQFATSLKGATLADVIAMAQQLNMTSRALRVEMEELSKLRMPCILHWELNHFVVLKKVRGNKITIHDPARGIRELTFKEASTAFTGVALELVPSSTFEVKEEKESISMMKLVGSVTGVKSAFAQVLILSIALELFGVLGPFFMQWVMDMVLVSADYSLLSLLGVGFIMIALFQTIVTALRSWVMSWFSSQLSVQWTINVCHHMLKLPLEWFESRHVGDILSRYGSLNTIQSTLTSRFISTVLDGVMSIVTVVMLFIYNAQLAWLVIGLFLAYALLRFMAYDPVRRANEEQIISSARTQSSLLETLRGIQAVKTNNKQVPRLSAYMNFLVDTTNKGIVIQKLNILFGSAQGLLTSVGRVVLVWLAALQVLDGNFSAGMLTAFISFSDQFMSRGSGLINAIIDFRMLRMHGERLADIVLSETEVSSEGNPGLVSKETDKETDVPQDIKLINLRFRYAPTEPWVVEGANLEIKAGESLAIVGPSGQGKTTMAKLILGLLHPEEGTMTVGGMDITQTGLEHHRNRIGCVMQDDILFSGSISENISFFDNEPDHAKITRVARLAQIHGDIMKMPMNYQSLVGDMGSFLSGGQLQRILLARALYREPKILVLDEATSHLDIYNEAQINNAIKQMKITRIIIAHRPETIRSADKIVLLNNGKLSEVTAEQLFGQTTTHNKTEITHG

SEQ ID NO: 682 - protein, 727 aa, synthetic construct
MRFGWKRRLPMMLQTQAAECGLVCVGMIANYFGHDMDLASLRRRFTTSLKGATLNDIMQMSNQLGLTPRALRLDLNDLAKLNRPCILHWEMNHFVVLKEVSRDKITVHDPARGIRDIPMDEVSRGFTGVALELMPSASFQPVAEKQSISMLKLIGGVIGIRSAFVQVLLISAALEVCGIISPFYMQWVMDQVLVSADVDLLTLLGIGFLMLVVFQNAIAALRSWVVTWFSSLLSVQWTASVCSHLLKLPMSYFEQRHMGDIVSRFGSVGTIQSMLTSRFISTLLDGVMASITLVMLFVYSPMLTWLVLGLFAVYALIRWIAYRPLRRANEDQIAYSARAQSQLLESIRGVQTIKLGNKQEARVATYTNSVVDMTNRGIAIQRLSISFSTLQGVISGIGRVVLIWLAARQVLQNEFSAGMLVAFISFADQFTSRASGLIDTVIDIRMLRLHGERLADIVLTEPESNMEGSVTRANLVEEAKDRAPGIEVSNLRFRYADTEPWVIKDCSFTVEPGESVALIGPSGQGKTTMAKLLLGLLMPQKGEIRVDGVDIKKLGMLGYRDMIGSVMQDDILFAGSIADNISFFDTHPDYAQVERVARIAQIHDEITAMPMGYQSLVGDMGSSLSGGQAQRVLLARALYRNPSILILDEATSHLDVDRERLINDAIRNMEVTRIIIAHRPETIRSADRIIAMQQGVAHELRAQQAGEPSTTFAAGRLDSGNVKITYP

SEQ ID NO: 683 - protein, 53 aa, synthetic construct
  REGION 1..53: note = Synthetic construct
NSDSECPLSHDGYCLHDGVCMYIEALDKYACNCVVGYIGERCQYRDLKWWELR

SEQ ID NO: 684 - protein, 70 aa, synthetic construct [sequence omitted]

SEQ ID NO: 685 - protein, 31 aa, synthetic construct
  REGION 1..31: note = Synthetic construct
YGGFMTSEKSQTPLVTLFKNAIIKNAHKKGQ

SEQ ID NO: 686 - protein, 20 aa, synthetic construct
  REGION 1..20: note = Synthetic construct
SFRNGVGSGAKKTSFRRAKQ

SEQ ID NO: 687 - protein, 36 aa, synthetic construct
  REGION 1..36: note = Synthetic construct
YPSKPDNPGEDAPAEDMARYYSALRHYINLITRQRY

SEQ ID NO: 688 - protein, 5 aa, synthetic construct
  REGION 1..5: note = Synthetic construct
YGGFL

SEQ ID NO: 689 - protein, 5 aa, synthetic construct
  REGION 1..5: note = Synthetic construct
YGGFM

SEQ ID NO: 690 - protein, 17 aa, synthetic construct
  REGION 1..17: note = Synthetic construct
YGGFLRRIRPKLKWDNQ

SEQ ID NO: 691 - protein, 33 aa, synthetic construct
  REGION 1..33: note = Synthetic construct
QPLPDCCRQKTCSCRLYELLHGAGNHAAGILTL

SEQ ID NO: 692 - protein, 58 aa, synthetic construct
  REGION 1..58: note = Synthetic construct
FVNQHLCGSHLVEALYLVCGERGFFYTPKTGGGPGKRGIVEQCCTSICSLYQLENYCN

SEQ ID NO: 693 - protein, 36 aa, synthetic construct
  REGION 1..36: note = Synthetic construct
SLEEEWAQVECEVYGRGCPSGSLDESFYDWFERQLG

SEQ ID NO: 694 - protein, 26 aa, synthetic construct
  REGION 1..26: note = Synthetic construct
CKGKGAKCSRLMYDCCTGSCRSGKCG

SEQ ID NO: 695 - protein, 70 aa, synthetic construct
  REGION 1..70: note = Synthetic construct
ARATNVGRECCLDYFKGAIPIRKLVSWYKTSVECSRDAIVFLTVQGKLICADPKDKHVKKAIRLVKNPRP

SEQ ID NO: 696 - protein, 88 aa, Burkholderia cepacia
MNYVDSGFACEAVGVCRELTEEELMIVAGGGNVGGAILGGLKGSVVGGIGGALGAAAVGAPVGAGGISGMVSGGIGGAVAGFSGGGGG

SEQ ID NO: 697 - protein, 89 aa, Salmonella enterica
MRELDSKELNIVGGAGDPLTDPNSQIMRQIMAGAAWGATFGVRGGLPGMAVGMVGGVTQTVVQGAAAQMPVNVPIPKVPMGPTWNGSKG

SEQ ID NO: 698 - protein, 112 aa, Kosakonia quasisacchari
MLSIRELNLEEIAMVSGGNANSNYEGGSSRGGSSNRSGSSHSSGKDIYSGVDSCGAGIFGGLVAGSLGGPAGMALGIVGGMIGGQCTKDSFSSKGGNGNKDKSNDFAGQCHW

SEQ ID NO: 699 - protein, 81 aa, Klebsiella indica
MRELVHDEIKSIAGAECTFNGLGQAAVSVGVAGAIGGTIVGNATLPVIGTVPGWVAGGLMGTIGGTAGYGATCWWSDTSTN

SEQ ID NO: 700 - protein, 110 aa, Enterobacter sp.
MSSIRELSFFEVAQVSGGGNAGDHDRSSSSNSGSKNSGSKNSNIYNGVDSCGAGIIGGLVAGSPGGPLGMAAGVIGGAIAGQCTKDSFSSKADCKGSNQKSSDFTGQCRW

SEQ ID NO: 701 - protein, 85 aa, Salmonella enterica
MRPISDTELSMISGAGEFSYGELAGVVAGGAVAGAMGGAVIGGLGAGPGAIAGATTAGAAYVAGSLVQSFFGNNSSATSSSGAGS

SEQ ID NO: 702 - protein, 79 aa, Raoultella ornithinolytica
MRELTQDQIENVAGAGGAPATVANATGVGIIIGSLGAIGAGIAAGTPVGPAGMIIGGILGGLGVAVGSATGSSGAGGGS

SEQ ID NO: 703 - protein, 122 aa, Escherichia sp.
MRELSLEEIGYVSGGNANSNYDSGRNTCSETNNNSNQSAGRSSSGGYYSGLGQDVTNCNNGIIGGMIAGSMGGFGGMVAGLVGGAVAGGCFSTSSSGGRNNNGGGTGNNSNCNSGGFGGVCR

SEQ ID NO: 704 - protein, 124 aa, Klebsiella pneumoniae
MSKVRELTIAEMNLVSGGGHFGDIHDAQGVGTGNKNNGGARNSLGRNAPTHIYSDPNTVNCANSVFSGMVGGAIKGGVVGMARGTIGGGVLGQCLSGGGNGNGGGNKAGSSNCSGSNVGGTCSR

SEQ ID NO: 705 - protein, 121 aa, Citrobacter sp.
MSIRELSLMEISCVSGGNANSNYESGRNAGSSSSRNNNKNSSSGNRSGFYGSLGQDVTNCNNGIIGGAIAGMMGGPAGMALGIMGGGLAGGCFSNGGNNSNGGGTGTNSNCNSGGMGGVCR

SEQ ID NO: 706 - protein, 70 aa, Escherichia coli
MRPLTENELKNVSGALYESVNTNCVLASGAMIVSAFGGPIGLISTGLAAIGACATSSSSSSASASAGNGS

SEQ ID NO: 707 - protein, 94 aa, Enterobacter cloacae
MRELNESELSNVSGAGMWGSIGSAIGGMFGGGSAGCNGSYSHTSTTNKDGSHSESTSFSGGCSVNVGGKSGGSSKGNGGSKSGGGRQGNDHGRH

SEQ ID NO: 708 - protein, 95 aa, Raoultella terrigena
MRALTEVELSFIYGADGNDSYNPISLIVGSLSTIAKLPTPVSLAVGIGTEIAIQASPHMPVHVPVPKIPMGPTWNGSGGGRPSPSASLSTPDSSN

SEQ ID NO: 709 - protein, 85 aa, Salmonella enterica
MRALTDNEVFSVYGADRGDSAVTGAVAGGTAGGAAGGWAGAELGAELGSLAGPIGTFVGFLGGAAIGAFGGAYIYDSFSSPSNGS

SEQ ID NO: 710 - protein, 104 aa, Escherichia coli
MRTLTLNELDSVSGGASGRDIAMAIGTLSGQFVAGGIGAAAGGVAGGAIYDYASTHKPNPAMSPSGLGGTIKQKPEGIPSEAWNYAAGRLCMDLPLYFQTPVIT

SEQ ID NO: 711 - protein, 104 aa, Salmonella enterica
MRELNEHEIKCAAGGQDYGREIAQGAGAAAGTFVAGGGGAIVGPIVAGMVYDWASNKTPDSALSPSGLGSTLKKKPEGLPSEAWNYAEGRMCNWSPNNLNDVCK

SEQ ID NO: 712 - protein, 89 aa, Salmonella enterica
MRELDNKELDFVGGAGDPLADPNSQILRQVMANAAWGAAFGIRGGLPGMAVGMAGGVAQTVIQGAAAQMPVNVPIPQVPMGPTWNGSKG

SEQ ID NO: 713 - protein, 90 aa, Salmonella enterica
MRNLTLNELKSISGAGDGDGVGRDIATALGGLAGGFVAGAVGAAAGGVAGGAVYDFASTHTVNPAMSPSGLGGYLGGYYGSSSSAVNSGS

SEQ ID NO: 714 - protein, 102 aa, Enterobacter sp.
MRELSQQDLVLAFGAGDANSQLISDMGNNAAWGAGLGAITGGGPGAAIGAVGGVVQTIGQGLIDHGPVSVPVPVMIGPSWNGSGGWSGAGFGSSSGGAGSAS

SEQ ID NO: 715 - protein, 88 aa, Leclercia adecarboxylata
MRKLNSKEMKLMGGAGDPLTDQNSQLIRQIMIGAGWGMMWGVPMGFPGMAAGAITATTQTVLQGAMSRMPVNVLIPRVPMGPTWNGSK

SEQ ID NO: 716 - protein, 107 aa, Escherichia coli
MRNITTDELALIHGAGLDKDASVAIGTAAGGFLGKATKIPSADIAGSAIGGYIGGVIADGSNRTITIPSNGINYNPSIGLGSFNPNYNGGLLNSGFNSSTSSSSSGS

SEQ ID NO: 717 - protein, 114 aa, Yokenella regensburgei
MSNIRELSFDEIASVSGGEGHGSEVAQDRRDARNSLGRNAPNKIYGGTEKCVNNILIGAGGGAVTGGLPGAAVGLVGGAYAGECLSGGGNGNGNGCNAGSNNCSSGNAASTCNR

SEQ ID NO: 718 - protein, 105 aa, Escherichia coli
MREITLNEMNNVSGAGDVNWVDVGKTVATNGAGVVGGAFGAGLCGPVCAGVFAVGSSAAVGALYDAAGNSKPTKQKPEGLPPEAWNYAEGRMCNWSPNNLNDVCL

SEQ ID NO: 719 - protein, 104 aa, Salmonella enterica
MRELNEHEIKCAAGGQDYGREIAQGAGAAAGTFVAGGGGAIVGPIVAGMVYDWASNKTPDSALSPSGLGSTLKQKPEGLPSEAWNYTEGRMCNWSPNNLNDVCK

SEQ ID NO: 720 - protein, 116 aa, Citrobacter amalonaticus
MRELSISEINFVSGGNANSNYENGRNTSSSRGSNYNNQGRNNNSGFYGGLGQDVTNCNNGIIGGAIAGSLGGLAGVAVGITGGAFAGGCFSHGNGNGGGSGANSNCSSSGIGGTCK

SEQ ID NO: 721 - protein, 91 aa, Pantoea agglomerans
MRELNSEEIRNVAGAGTDNPYDNPILLMNQIAENAAWGAALGAYGGPEGVILGAGGGVVQTVVQGAAAQMPVNVPIPQVPLGPTWNGSGSN

SEQ ID NO: 722 - protein, 114 aa, Mixta gaviniae
MRMLINNDIKLVTGAGANTATEYVGHMKNADKYAGGSGSGNKGGGFYTSQGVVDCNNGIIGGVIMGAYGLGWGMALGVVGGALAGGCFKTEGGNGAGGGSGSNGSNCNDGGCSW

SEQ ID NO: 723 - protein, 119 aa, Pantoea allii
MLRELTDSEINIVSGGNANSGYEGPNCTNAAIKGGVTGATGGAVAGAIRGGVTGLAGGPAGVVAGAGTGAAEGAVYGGVSGAVGGYVGCQNSGSANNGGNNGNLGGDSNANSANGQCHW

SEQ ID NO: 724 - protein, 104 aa, Erwinia mallotivora
MENINTVLMEKVSGAGNYADKHEIGNSSGRGGAPDSCANRAGLGAIIGTLGALATGATGGLAGVVVAGVTGGLSSVIGCENPSYNDNNSGNSSRSGNYGAQCTW

SEQ ID NO: 725 - protein, 106 aa, Vibrio sp.
MRELNNNEILIVDGGGERGGGDHSGEWSSWSGSYSNNSNSSNNSNYPASEQAVQQQANSPTQDCAQAVAGNAIGGAIAGSGGGLAGAGLGALGGGLAGALGGSCNQ

SEQ ID NO: 726 - protein, 90 aa, Vibrio cholerae
MKELSIEQISLVTGGERGDGRDRHSLLHELKDIAQYLITRNPLGLITHSEPLNAGEEAWLKEREREGARHGYNERQGGYHRGDGGRHIEN

SEQ ID NO: 727 - protein, 91 aa, Vibrio cholerae
MKTLTLDEMKIVSGGRHAEGAYNANGPDRTREQARHENYPGREWSSADLRNAGLALSAVGAVTPGVPGRVIGALGAGAALIGGWERNGGNH

SEQ ID NO: 728 - protein, 89 aa, Vibrio splendidus
MRSLNASEVNMVSGGREARGAYNSNGPDRTRAQAMRENNPGRDWSNSDLNNVSLALGLAGTLVPGIWGRGISALGAAGAAVSGWKRKDK

SEQ ID NO: 729 - protein, 70 aa, Vibrio thalassae
MYNLSADEISMVDGGGDGNSSGYPASPSAVAQQANSPSRACGQAIATGAVVNGMAGGYMGGLKGAVAGAV

SEQ ID NO: 730 - protein, 58 aa, synthetic construct
  REGION 1..58: note = Synthetic construct
VDNKFNKETQEASWEIFTLPNLNGRQVAAFISSLLDDPSQSANLLAEAKKLNDAQAPK

SEQ ID NO: 731 - protein, 58 aa, synthetic construct
  REGION 1..58: note = Synthetic construct
VDNKFNKEQQNAFYEILHLPNLNEVQVKAFIDSLRDDPSQSANLLAEAKKLNDAQAPK

SEQ ID NO: 732 - protein, 58 aa, synthetic construct
  REGION 1..58: note = Synthetic construct
VDNKFNKENIAAMTEITRLPNLNPYQRAAFIWSLKDDPSQSANLLAEAKKLNDAQAPK

SEQ ID NO: 733 - protein, 58 aa, synthetic construct; identical to SEQ ID NO: 732
  REGION 1..58: note = Synthetic construct

SEQ ID NO: 734 - protein, 58 aa, synthetic construct
  REGION 1..58: note = Synthetic construct
VDAKYAKEWMRAAHEIRWLPNLTFDQRVAFIHKLEDDPSQSSELLSEAKKLNDSQAPK

SEQ ID NO: 735 - protein, 58 aa, synthetic construct
  REGION 1..58: note = Synthetic construct
VDNKFNKELGWAIGEIGTLPNLNHQQFRAFILSLWDDPSQSANLLAEAKKLNDAQAPK

(19) United States
(12) Patent Application Publication
(10) Pub. No.: US 2025/0340892 A1
(43) Pub. Date: Nov. 6, 2025

(54) GRAM-NEGATIVE BACTERIA CONTAINING PEPTIDE SECRETION SYSTEM

(71) Applicant: Board of Regents, the University of Texas System, Austin, TX (US)

(72) Inventors: Bryan DAVIES, Austin, TX (US); Sun-Young KIM, Austin, TX (US); Timothy Jeffrey COLE, Austin, TX (US); Jennifer PARKER, Austin, TX (US); Mady TELFORD, Austin, TX (US)

(73) Assignee: Board of Regents, the University of Texas System, Austin, TX (US)

(21) Appl. No.: 18/290,591

(22) PCT Filed: Jul. 22, 2022
(86) PCT No.: PCT/US2022/038064

§ 371 Date: Jan. 19, 2024

Related U.S. Application Data
(60) Provisional application No. 63/225,311, filed on Jul. 23, 2021.
(60) Provisional application No. 63/348,904, filed on Jun. 3, 2022.

(51) Int. Cl. C12N 15/74; A01N 63/20; C07K 14/245

(52) U.S. Cl. CPC C12N 15/74; A01N 63/20; C07K 14/245; C07K 2319/10; C07K 2319/50

(57) ABSTRACT

Gram-negative bacterial cells for expression and secretion of heterologous peptides are disclosed. The bacterial cells include a first nucleic acid encoding a secretion signal sequence, e.g., a microcin secretion signal sequence, fused to a heterologous peptide, a second nucleic acid encoding a C39 peptidase-containing ATP-binding cassette transporter (PCAT), and a third nucleic acid encoding a membrane fusion protein. The gram-negative bacterial cells secrete the heterologous peptide from the bacterial cytosol to an external environment outside of the bacterial outer membrane. Methods for preparation and delivery of peptides to external environments are also described.

### PRIOR RELATED APPLICATIONS

This application claims the benefit of U.S. Provisional Application No. 63/225,311, filed on Jul. 23, 2021, and U.S. Provisional Application No. 63/348,904, filed on Jun. 3, 2022, which are hereby incorporated by reference in their entireties.

### STATEMENT AS TO RIGHTS TO INVENTIONS MADE UNDER FEDERALLY SPONSORED RESEARCH AND DEVELOPMENT

This invention was made with government support under Grant nos. R01 AI125337 and R01 AI148419 awarded by the National Institutes of Health, Grant no. W911NF-16-1-0146 awarded by the Army Research Office, and Grant no. HR0011-19-2-0011 awarded by the Defense Advanced Research Projects Agency (DARPA). The government has certain rights in the invention.

### BACKGROUND OF THE INVENTION

Engineered microorganisms for secretion of recombinant peptides have been successfully utilized in cost-effective peptide production, drug discovery, and delivery of therapeutic peptides [see, Yaginuma, et al. Scientific Reports 9, 1-11 (2019); Chen, et al. Scientific Reports 7, (2017); Xu, et al. Asian-Australas J Anim Sci 30, 576-584 (2017); Geldart, et al. Bioeng Transl Med 3, 197-208 (2018)]. However, engineering gram-negative bacteria to secrete recombinant peptides is difficult. Gram-negative bacteria have an additional outer membrane (OM), which acts as a barrier for extracellular secretion of a target peptides via general secretory pathways [see, Wegmüller, et al. Current Organic Chemistry 18, (2014); Burdette, et al. Microbial Cell Factories 17, 196 (2018)]. Previous studies have developed specific ways to secrete target peptides from gram-negative E. coli. These methods include conjugation of target peptides with a type three secretion system (T3SS) signal peptide, a super folding GFP (green fluorescence protein), or YebF [see, Yu, et al. JCI Insight 4, (2019); Seo, E., et al. Int. J. Med. Microbiol. 302, 276-287 (2012). Zhang, Z. et al. Sci Rep 7, 6990 (2017)]. Such strategies are of limited utility as a general method for secretion of heterologous peptides in gram-negative bacteria. For example, the secreted peptides will frequently include signal peptides or fusion partners, which may alter structure or activity of the target peptide. In addition, the strict substrate specificity of many transport systems precludes the secretion of heterologous peptides and/or use in heterologous microbes.

### BRIEF SUMMARY OF THE INVENTION

Provided herein are gram-negative bacterial cells comprising: a first nucleic acid encoding a secretion signal sequence fused to a heterologous peptide, a second nucleic acid encoding a C39 peptidase-containing ATP-binding cassette transporter (PCAT), and a third nucleic acid encoding a membrane fusion protein; and an inner membrane surrounding cytosol, an outer membrane surrounding the inner membrane, and periplasmic space between the inner membrane and the outer membrane; wherein the gram-negative bacterial cell secretes the heterologous peptide from the bacterial cytosol to an external environment outside of the outer membrane.

Also provided herein are methods for preparing peptides. The methods include: culturing a gram-negative bacterial cell as described herein such that the heterologous peptide is expressed and secreted from the cytosol to the external environment; and isolating the secreted heterologous peptide, thereby preparing the peptide.

Also provided herein are methods for delivering a peptide from gram-negative bacterial cytosol to an external environment. The methods include introducing a gram-negative cell as described herein to the external environment, such that the gram-negative bacterial cell expresses and secretes the heterologous peptide from the cytosol to the external environment, thereby delivering the peptide. The external environment may be, for example, an agricultural environment or an organ or tissue in a human subject or animal subject.

Also provided are gram-negative epiphytic or soil bacterial cells comprising: a first nucleic acid encoding a secretion signal sequence fused to a heterologous agricultural peptide, a second nucleic acid encoding a C39 peptidase-containing ATP-binding cassette transporter (PCAT), and a third nucleic acid encoding a membrane fusion protein; and an inner membrane surrounding cytosol, an outer membrane surrounding the inner membrane, and periplasmic space between the inner membrane and the outer membrane; wherein the gram-negative bacterial cell secretes the heterologous agricultural peptide from the bacterial cytosol to an external environment outside of the outer membrane.

Also provided herein are methods for preparing agricultural peptides. The methods include: culturing a gram-negative gram-negative epiphytic or soil bacterial cell as described herein such that the heterologous agricultural peptide is expressed and secreted from the cytosol to the external environment; and isolating the secreted heterologous agricultural peptide, thereby preparing the peptide.

Further provided herein are methods for preparing an agricultural peptide. The methods include: culturing a gram-negative epiphytic bacterial cell or soil bacterial cell described herein, such that the heterologous agricultural peptide is expressed and secreted from the cytosol to the external environment; and isolating the secreted heterologous agricultural peptide, thereby preparing the peptide.

Also provided are methods for delivering a heterologous agricultural peptide from a gram-negative epiphytic or soil bacteria cytosol to a plant, the method comprising contacting the plant with any of the gram-negative epiphytic bacterial cells described herein, such that the gram-negative epiphytic bacterial cell expresses and secretes the heterologous peptide from the cytosol to the plant, thereby delivering the peptide

### BRIEF DESCRIPTION OF THE DRAWINGS

FIG. 1 shows the construction of an exemplary secretion system according to the present disclosure. (A) The secretion machinery complex embedded in the cytoplasm, inner membrane (IM), periplasm and outer membrane (OM) consist of three proteins (CvaB, CvaA and TolC) is depicted. The 15-amino-acid signal peptide (SP) sequences of the MccV is cleaved by peptidase domain (PEP) of CvaB during export. (B) Plasmids for positive and negative secretion are shown. Positive secretion comprised of a plasmid (pBAD18-Km derived) expressing MccV's SP conjugated peptide of interest (POI) and a plasmid (pACYC184 derived) expressing CvaA and CvaB. Negative secretion expresses POI same as positive secretion, but does not express CvaA/CvaB or express CvaA/CvaB C32S, named no AB and C32S, respectively. (C) The result of agar diffusion assay is shown. E. coli W3110 cultures containing positive and negative secretion of MccV spotted on the E. coli W3110 WT lawn plate. The picture is a representative image of a biological duplicate, and cropped from the same plate image. MccV: Microcin V, pTc: promoter region for tetracycline resistant gene, WT: Wild-type. +/−: presence and absence, respectively.

FIG. 2 shows assessment of recombinant peptide secretion via the MccV system. (A) Western blot for detecting secreted and intracellular expressed MccV_V5 in E. coli W3110. Supernatant or pellet of culture directly suspended in sample buffer and loaded to each well. Details of sample in each lane are described as a presence/absence of components table. Antibody targets are described in the left side. (B) The result of dot blot against V5 tag is shown. Supernatant or whole cell lysate samples were directly loaded into wells of dot blot apparatus containing nitrocellulose membrane. (C) Agar diffusion assay was performed as described in FIG. 1C. All results are a representative image of biological duplicate. WT: Wild-type. +/−: presence and absence, respectively.

FIG. 3 shows the properties of random synthetic peptides. (A) Theoretical charge (at pH=7.0) and hydrophobicity of peptides are shown as a scatter plot. Different group represented as a different color; Group 1 as red, Group 2 as blue, Group 3 as green and Group4 as magenta. (B) Distances between each plotted peptide and a point (charge=0, hydrophobicity=0) were calculated and represented. the Line represents median value. Unpaired t-test result are shown as ns (not significant) or the number of asterisks (*=P<0.05, **=P<0.005) (C) The composition of amino acids belonging a particular class was calculated per group. Details of each class are available at cran.r-project.org/web/packages/Peptides/Peptides.pdf. The representing color for each group is the same as (A).

FIG. 4 shows secretion levels of random peptides. (A) Dot blot result for measuring V5 tag signal intensities supernatant or cell lysate samples of random peptides is shown. The group of random peptide is shown in left side, and the number of top side represents each peptide member in the group. Supernatant samples contained both positive and negative secretion (no AB). Cellular expression was measured using cultures of negative secretion (no AB). V5 tag signal of empty vector (EV) culture lysate sample is also shown. Detail is described in method and material section. (B) Calculated secretion level was graphed. Different group represented as a different color; Group 1 as red, Group 2 as blue, Group 3 as green and Group 4 as magenta. Mean of biological triplicate with standard deviation (SD) was shown. (C) Secretion level per group is shown as a scatter plot. Line represents median value. Unpaired t-test results between group 4 and other groups are shown as the number of asterisks (*=P<0.05, **=P<0.005). (D,E,F) Linear regression analysis between secretion level and charge, hydrophobicity and expression level was performed. R square values are shown in each graph.

FIG. 5 shows an assessment the MccV system's secretion capacity. (A) Dot blot result for measuring V5 tag signal intensities of standard peptides (NPS_V5 and ECP_V5), positive and negative secretion of selected peptides (MccV_V5, G1P9, G2P9, G3P2 and G4P7) is shown. Both positive and negative supernatants samples (data not shown) were diluted 1:25 into fresh LB medium and loaded into dot blot apparatus wells. Triplicate supernatant samples and duplicate each standard peptide with serial 2-fold dilution were loaded. Total amounts (ng) of standard peptides diluents in each well are shown. (B) Linear Standard curve of NPS_V5 and ECP_V5. The concentration of two standard peptides (ng/ml) was converted to μM, and the mean of two peptides' signal intensity was plotted. R square value is shown. (C) Absolute concentrations of selected peptides were calculated by standard equation (y=572678*x+1863). Mean of biological triplicate with standard deviation (SD) is shown. rep: replication.

FIG. 6 shows the effects of peptide size on secretion via the MccV system. (A) Dot blot result for measuring V5 tag signal intensities of G1P6, G1P6_2X, G3P2, G3P2_2X supernatant and whole cell lysate samples is shown. Details of sample are described as a table of presence/absence of CvaA/CvaB proteins. V5 tag signal of empty vector (EV) culture lysate sample is also shown. (B,C,D) Secretion or expression levels of samples are graphed. Mean of three replicates is shown with standard deviation (SD). Unpaired t-test results are shown as ns (not significant=P>0.05), or the number of asterisks (*=P<0.05, **=P<0.005, ***, ****=P<0.0005). Expression levels below zero are shown as not available (N/A). Rep: replication, +/−: presence and absence, respectively.

FIG. 7 shows the secretion of bioactive peptides via the MccV system. (A) The results of two agar diffusion assays are shown. In upper panel, empty vector, Pediocin PA-1 positive and negative secretion E. coli cultures are spotted onto an agar plate containing L. monocytogenes and 0.2% (w/v) of arabinose. In lower panel, empty vector, «-factor positive and negative secretion E. coli cultures are spotted onto an agar plate containing S. cerevisiae and 0.2% (w/v) of arabinose. Each picture is the representative image of biological triplicate and cropped from the same plate image. (B) The result of neutrophil elastase (NE) activity assay is shown. Fluorescence levels (excited at 400 nm and emits at 505 nm) of samples at each time point are measured. The sample names represent what is included. NE represents the mixture of NE and its substrate. NE+Eglin C PS represents the mixture of NE, substrate, and Eglin C positive secretion sample. NE+Eglin C NS represents the mixture of NE, substrate and Eglin C negative secretion sample. NE+Eglin C (1.25 μM) represent the mixture of NE, substrate and 1.25 μM of purified recombinant Eglin C. (C) The result of Colorimetric ELISA result against EGF. A450 values indicates relative amount of EGF in samples. EGF (1 ng/ml) represents a sample containing purified EGF, Empty well represent a sample containing nothing other than assay buffer, EGF PS 1:100 represents a sample containing 100-fold diluted EGF positive secretion (PS) supernatant in fresh Ham's F-12 media. EGF NS represents a sample containing EGF negative secretion (NS) supernatant. G3P2 PS represents a sample containing G3P2 positive secretion (PS) supernatant. Mean of biological duplicate with standard deviation is shown. (D) Either EGFR (Epidermal growth factor receptor) transfected or non-transfected CHO cells were treated with each sample (Con: control (100 ng/ml of purified EGF), EGF PS: EGF positive secretion, NSP PS: non-specific peptide (G3P2) positive secretion) and the cell lysates were subjected to western blot against HA tag, pY1068 and β-Actin. pEGFR expression levels based on pY1068 antibody signal were normalized per EGF expression level and the relative expression levels are shown in the image. The blots are representative images of a biological duplicate. *RFU: Relative fluorescence level, OD: Optical density, min: minutes, +/−: presence and absence, respectively.

FIG. 8 shows the compatibility of the MccV system with various gram-negative bacteria. (A) Plasmids for broad-host-range positive and negative secretion are shown. (B) The result of agar diffusion assay is shown. The culture of Pediocin PA-1 positive or negative secretion samples from three different bacteria were spotted on a agar plate containing L. monocytogenes and 1 mM of IPTG. Each picture is a representative image of a biological duplicate. *SP: signal peptide, POI: peptide of interest, +/−: presence and absence, respectively.

FIG. 9 shows recombinant peptide secretion via the MccV system. (A) Zone of inhibition assays were performed as described in FIG. 1C. Empty vector (EV) strain carries empty plasmids, pBAD18 and pACYC184. Positive secretion (PS) strain encodes MccV_V5, Cvi and CvaAB. Negative secretion (NS) strain encodes MccV_V5, Cvi and empty pACYC184. Protease-deficient (PD) secretion strain encodes MccV_V5, Cvi and CvaA/CvaB C32S. All samples were spotted on the same agar plate. The result is representative of biological triplicate experiments. (B) Western blot result detecting secreted MccV_V5 from E. coli W3110 is shown. Culture supernatant or pellet (total cell lysate) were directly suspended in sample buffer and loaded to each well. Antibody targets are described in the right side. (C) The result of dot blot against V5 tag is shown. Supernatants from respective strains (EV, PS, NS and PD) were directly loaded into wells of dot blot apparatus containing nitrocellulose membrane. The result is representative of biological triplicates and all western or dot blot images were prepared from the same membrane.

FIG. 10 shows that bacteria secrete microcin HUW04 in the presence of its immunity protein or a defective immunity protein (S/A). In the presence of defective immunity protein, microcin HUW04 kills the bacteria observed by no change in growth. In the presence of the immunity protein protecting against HUW04 activity, the bacteria grows exponentially.

FIG. 11 shows that microcin HUW04 kills bacteria when immunity protein is off, as evident by the zone of clearance. The control peptide has not effect in either case.

FIG. 12 is a Coomassie stained gel showing purification of an exemplary affibody, ZpA from bacteria supernatant after secretion using the MccV system described herein.

FIG. 13 is a Western blot showing mCCL21a after secretion from bacteria. Chemokine was detected with an anti-CCL21a primary antibody and visualized with an HRP-conjugated secondary antibody.

FIG. 14 shows that application of culture supernatant from E. coli expressing an exemplary agricultural peptide (e.g., miPEP858a), enhances root growth in Arabidopsis thaliana compared to E. coli culture supernatant alone.

### DETAILED DESCRIPTION OF THE INVENTION

The present invention is based, in part, on the discovery that the secretion system for Microcin V (MccV; formerly known as Colicin V) can be used for secretion of a variety of heterologous peptides by E. coli and other gram-negative bacteria. As described in more detailed below, host cells engineered to contain the MccV system were used for recombinant expression and secretion of peptides including, but not limited to, MccV, Pediocin-PA1, α-factor, and Eglin C. The secretion efficiencies of various synthetic peptides were profiled to understand the effects of peptide properties on secretion. To the best of the inventors' knowledge, this work is the first comprehensive study of the MccV system in heterologous peptide secretion, and the first application of secretion systems having C39 peptidase-containing ATP-binding cassette transporters (PCATs) as a general platform for secretion of peptides, including non-membrane proteins exhibiting diverse functionality beyond bacteriocin or microcin activity, in gram-negative bacteria.

### I. Definitions

The terms “peptide,” “polypeptide,” and “protein” are used interchangeably herein to refer to a polymer of amino acid residues. All three terms apply to naturally occurring amino acid polymers and non-natural amino acid polymers, as well as to amino acid polymers in which one (or more) amino acid residue is an artificial chemical mimetic of a corresponding naturally occurring amino acid. As used herein, the terms encompass amino acid chains of any length, including full-length proteins, wherein the amino acid residues are linked by covalent peptide bonds. As used herein the term “agricultural peptide” refers to a peptide that modulates one or more plant properties. For example, and not to be limiting, an agricultural peptide can improve plant growth, plant development, plant disease resistance (for example, fungal or bacterial disease resistance), pigmentation, flower development, and/or stress tolerance. Examples of plant stress include, but are not limited to, environmental stress, mechanical stress, drought stress, salinity stress, hypoxia, light stress, temperature (e.g., for example, heat or cold) stress, chemical stress, pollution, and toxicity).

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either the commonly known three letter symbols or by the one-letter symbols recommended by the IUPAC-IUB Biochemical Nomenclature Commission. Nucleotides, as described herein, may also be referred to by their commonly accepted single-letter codes.

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

The terms “amino acid modification” and “amino acid alteration” refer to a substitution, a deletion, or an insertion of one or more amino acids. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

The terms “nucleic acid,” “nucleotide,” and “polynucleotide” refer to deoxyribonucleic acids (DNA) or ribonucleic acids (RNA) and polymers. The term includes, but is not limited to, single-, double-, or multi-stranded DNA or RNA, genomic DNA, cDNA, and DNA-RNA hybrids, as well as other polymers comprising purine and/or pyrimidine bases or other natural, chemically modified, biochemically modified, non-natural, synthetic, or derivatized nucleotide bases. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), orthologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “nucleotide sequence encoding a peptide” and “gene” refer to the segment of DNA involved in producing a peptide chain. In addition, a gene will generally include regions preceding and following the coding region (leader and trailer) involved in the transcription/translation of the gene product and the regulation of the transcription/translation. A gene can also include intervening sequences (introns) between individual coding segments (exons). Leaders, trailers, and introns can include regulatory elements that are necessary during the transcription and the translation of a gene (e.g., promoters, terminators, translational regulatory sequences such as ribosome binding sites and internal ribosome entry sites, enhancers, silencers, insulators, boundary elements, replication origins, matrix attachment sites and locus control regions, etc.). A “gene product” can refer to either the mRNA or protein expressed from a particular gene.

“Percentage of sequence identity” is determined by comparing two optimally aligned sequences over a comparison window, wherein the portion of the sequence (e.g., a peptide of the invention) in the comparison window may comprise additions or deletions (i.e., gaps) as compared to the reference sequence which does not comprise additions or deletions, for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

“Identical” and “identity,” in the context of two or more nucleic acids or polypeptide sequences, refer to two or more sequences or subsequences that are the same. Sequences are “substantially identical” to each other if they have a specified percentage of nucleotides or amino acid residues that are the same (e.g., at least 75%, at least 80%, at least 85%, at least 90%, at least 95%, or at least 99% identical over a specified region), when compared and aligned for maximum correspondence over a comparison window, or designated region as measured using one of the following sequence comparison algorithms or by manual alignment and visual inspection. These definitions also refer to the complement of a nucleic acid test sequence.

“Similarity” and “percent similarity,” in the context of two or more polypeptide sequences, refer to two or more sequences or subsequences that have a specified percentage of amino acid residues that are either the same or similar as defined by a conservative amino acid substitutions (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% similar over a specified region), when compared and aligned for maximum correspondence over a comparison window, or designated region as measured using one of the following sequence comparison algorithms or by manual alignment and visual inspection. Sequences are “substantially similar” to each other if, for example, they are at least 20%, at least 25%, at least 30%, at least 35%, at least 40%, at least 45%, at least 50%, or at least 55% similar to each other.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

Additional examples of algorithms that are suitable for determining percent sequence identity and sequence similarity are the BLAST and BLAST 2.0 algorithms, which are described in Altschul et al., (1990) J. Mol. Biol. 215:403-410 and Altschul et al. (1977) Nucleic Acids Res. 25:3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or peptides are substantially identical is that the peptide encoded by the first nucleic acid is immunologically cross reactive with the antibodies raised against the peptide encoded by the second nucleic acid. Thus, a peptide is typically substantially identical to a second peptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “expression” and “expressed” in the context of a gene refer to the transcriptional and/or translational product of the gene. The level of expression of a DNA molecule in a cell may be determined on the basis of either the amount of corresponding mRNA that is present within the cell or the amount of protein encoded by that DNA produced by the cell.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “constitutive promoter” is one that is capable of initiating transcription under most environmental conditions suitable for cell growth/propagation. An “inducible promoter” is one that initiates transcription only under particular environmental conditions or developmental conditions.

A polynucleotide/polypeptide sequence is “heterologous” to an organism or a second polynucleotide/polypeptide sequence if it originates from a different species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety). A heterologous promoter may also be a fully synthetic promoter, having a non-naturally occurring nucleotide sequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. For example, recombinant cells express genes that are not found within the native (non-recombinant) form of the cell or express native genes that are otherwise abnormally expressed, under-expressed, or not expressed at all.

An “expression cassette” refers to a nucleic acid construct, which when introduced into a host cell, results in transcription and/or translation of a RNA or polypeptide, respectively. Antisense constructs or sense constructs that are not or cannot be translated are expressly included by this definition. One of skill will recognize that the inserted polynucleotide sequence need not be identical, but may be only substantially similar to a sequence of the gene from which it was derived.

The terms “vector” and “recombinant expression vector” refer to a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain.

### II. Peptide Secretion Systems

Peptide excretion systems according to the present disclosure include components of export machinery present in various species of bacteria. The MccV system, for example, is present in various E. coli strains and contains a peptidase-containing ATP-binding cassette protein CvaB, a membrane fusion protein CvaA, and an outer membrane protein TolC [see, Vassiliadis, et al. “Class II Microcins” in Prokaryotic Antimicrobial Peptides: From Genes to Applications (eds. Drider, D. & Rebuffat, S.) 309-332 (Springer New York, 2011); Zhang, et al. Genetics 141, 25-32 (1995)]. The system secretes its cognitive substrate, MccV, an anti-bacterial polypeptide, directly from cytoplasm to extracellular space in a signal peptide-mediated way; MccV is synthesized as a 103-amino-acid precursor product containing an N-terminal 15-amino-acid signal peptide sequence (CvaC15), and the peptidase domain of CvaB cleaves the signal peptide sequence in an ATP binding-hydrolysis manner to release the substrate from the complex to extracellular space [see, Smith, et al. Journal of Bacteriology 200, e00168-18 (2018)].

Accordingly, some embodiments of the present disclosure provide gram-negative bacterial cells comprising:
a first nucleic acid encoding a secretion signal sequence fused to a heterologous peptide, a second nucleic acid encoding a C39 peptidase-containing ATP-binding cassette transporter (PCAT), and a third nucleic acid encoding a membrane fusion protein; and
an inner membrane surrounding cytosol, an outer membrane surrounding the inner membrane, and periplasmic space between the inner membrane and the outer membrane;
wherein the gram-negative bacterial cell secretes the heterologous peptide from the bacterial cytosol to an external environment outside of the outer membrane.

A variety of heterologous peptides can be expressed and secreted by the gram-negative bacterial cells of the present disclosure. Typically, the heterologous peptide will contain from about 5 amino acid residues to about 150 amino acid residues. The heterologous peptide may contain, for example, 5-15 amino acid residues, or 15-25 amino acid residues, or 25-35 amino acid residues, or 35-45 amino acid residues, or 45-55 amino acid residues, or 55-65 amino acid residues, or 65-75 amino acid residues, or 75-85 amino acid residues, or 85-95 amino acid residues, or 95-105 amino acid residues, or 105-115 amino acid residues, or 115-125 amino acid residues, or 125-135 amino acid residues, or 135-145 amino acid residues, or 145-150 amino acid residues.

In some embodiments, the heterologous peptide is a non-membrane protein (e.g., a cytosolic protein or an extracellular protein). In some embodiments, the heterologous peptide is a growth factor, a pheromone, a hormone, a neuropeptide, a protease inhibitor, a self-assembling peptide, or a cell-signaling peptide. In some embodiments, the heterologous peptide is a heterologous peptide set forth in Table 1.

TABLE 1

| | | SEQ | |
|---|---|---|---|
| | | ID | |
| Peptide | Origin | NO: | Sequence |
| EGF | Human | 683 | NSDSECPLSHDGYCLHDGVCM |
| | | | YIEALDKYACNCVVGYIGERCQ |
| | | | YRDLKWWELR |
| Eglin C | Medical Leech | 684 | TEFGSELKSFPEVVGKTVDQAR |
| | | | EYFTLHYPQYDVYFLPEGSPVT |
| | | | LDLRYNRVRVFYNPGTNVVNH |
| | | | VPHVG |
| β-Endorphin | Mouse | 685 | YGGFMTSEKSQTPLVTLFKNAII |
| | | | KNAHKKGQ |
| Neuropeptide S | Mouse | 686 | SFRNGVGSGAKKTSFRRAKQ |
| Neuropeptide Y | Mouse | 687 | YPSKPDNPGEDAPAEDMARYYS |
| | | | ALRHYINLITRQRY |
| Leu-enkephalin | Mouse | 688 | YGGFL |
| Met-enkephalin | Mouse | 689 | YGGFM |
| Dynorphin A 1-17 | Mouse | 690 | YGGFLRRIRPKLKWDNQ |
| Orexin A | Mouse | 691 | QPLPDCCRQKTCSCRLYELLHG |
| | | | AGNHAAGILTL |
| Single chain | Synthetic | 692 | FVNQHLCGSHLVEALYLVCGER |
| Insulin(HsC13R) | | | GFFYTPKTGGGPGKRGIVEQCC |
| | | | TSICSLYQLENYCN |
| S519 | Synthetic | 693 | SLEEEWAQVECEVYGRGCPSGS |
| | | | LDESFYDWFERQLG |
| conotoxin | Snail | 694 | CKGKGAKCSRLMYDCCTGSCR |
| | | | SGKCG |
| Chemokine CCL17 | Mouse | 695 | ARATNVGRECCLDYFKGAIPIR |
| | | | KLVSWYKTSVECSRDAIVFLTV |
| | | | QGKLICADPKDKHVKKAIRLVK |
| | | | NPRP |

In some embodiments, the heterologous peptide is an antimicrobial peptide, for example, a microcin set forth in Table 2, which have been validated in the systems described herein. In some embodiments, the heterologous peptide is not an antimicrobial peptide (e.g., not a bacteriocin or microcin naturally expressed by a bacterium or other microbe).

TABLE 2
Microcins

| | | Microcin | | |
|---|---|---|---|---|
| | | Protein | | |
| | | Accession | SEQ ID | |
| Microcin | Source Strain | (Uniprot) | NO: | Pre-Microcin Sequence |
| BK12 | Burkholderia | | 696. | MNYVDSGFACEAVGVCRELTEEELMIVAGG |
| | cepacia | | | GNVGGAILGGLKGSVVGGIGGALGAAAVGA |
| | MSMB1906 | | | PVGAGGISGMVSGGIGGAVAGFSGGGGG |
| EN101 | Salmonella | A0A5U4CZP4 | 697. | MRELDSKELNIVGGAGDPLTDPNSQIMRQIM |
| | enterica | | | AGAAWGATFGVRGGLPGMAVGMVGGVTQ |
| | (Salmonella | | | TVVQGAAAQMPVNVPIPKVPMGPTWNGSK |
| | choleraesuis) | | | G |
| | 2015K-0757 | | | |
| EN112 | Kosakonia | A0A4V2LWI9 | 698. | MLSIRELNLEEIAMVSGGNANSNYEGGSSRG |
| | quasisacchari | | | GSSNRSGSSHSSGKDIYSGVDSCGAGIFGGLV |
| | WCHEs120001 | | | AGSLGGPAGMALGIVGGMIGGQCTKDSFSS |
| | | | | KGGNGNKDKSNDFAGQCHW |
| EN123 | Klebsiella | A0A5R9L901 | 699. | MRELVHDEIKSIAGAECTFNGLGQAAVSVGV |
| | indica | | | AGAIGGTIVGNATLPVIGTVPGWVAGGLMG |
| | TOUT106 | | | TIGGTAGYGATCWWSDTSTN |
| EN124 | Enterobacter sp. | A0A7W2W022 | 700. | MSSIRELSFFEVAQVSGGGNAGDHDRSSSSN |
| | RHBSTW- | | | SGSKNSGSKNSNIYNGVDSCGAGIIGGLVAG |
| | 00974 | | | SPGGPLGMAAGVIGGAIAGQCTKDSFSSKAD |
| | | | | CKGSNQKSSDFTGQCRW |
| EN126 | Salmonella | A0A5Y3MYM1 | 701. | MRPISDTELSMISGAGEFSYGELAGVVAGGA |
| | enterica subsp. | | | VAGAMGGAVIGGLGAGPGAIAGATTAGAA |
| | salamae 275803 | | | YVAGSLVQSFFGNNSSATSSSGAGS |
| EN134 | Raoultella | A0A225U078 | 702. | MRELTQDQIENVAGAGGAPATVANATGVGII |
| | ornithinolytica | | | IGSLGAIGAGIAAGTPVGPAGMIIGGILGGLG |
| | (Klebsiella | | | VAVGSATGSSGAGGGS |
| | ornithinolytica) | | | |
| | TET32 | | | |
| EN138 | Escherichia sp. | A0A659L988 | 703. | MRELSLEEIGYVSGGNANSNYDSGRNTCSET |
| | E2586 | | | NNNSNQSAGRSSSGGYYSGLGQDVTNCNNG |
| | | | | IIGGMIAGSMGGFGGMVAGLVGGAVAGGCF |
| | | | | STSSSGGRNNNGGGTGNNSNCNSGGFGGVC |
| | | | | R |
| EN143 | Klebsiella | A0A2L0KUU8 | 704. | MSKVRELTIAEMNLVSGGGHFGDIHDAQGV |
| | pneumoniae | | | GTGNKNNGGARNSLGRNAPTHIYSDPNTVN |
| | CPE7 | | | CANSVFSGMVGGAIKGGVVGMARGTIGGGV |
| | | | | LGQCLSGGGNGNGGGNKAGSSNCSGSNVGG |
| | | | | TCSR |
| EN153 | Citrobacter sp. | A0A0J1N4I2 | 705. | MSIRELSLMEISCVSGGNANSNYESGRNAGS |
| | MGH109 | | | SSSRNNNKNSSSGNRSGFYGSLGQDVTNCNN |
| | | | | GIIGGAIAGMMGGPAGMALGIMGGGLAGGC |
| | | | | FSNGGNNSNGGGTGTNSNCNSGGMGGVCR |
| EN16 | Escherichia coli | A0A4T9HPU8 | 706. | MRPLTENELKNVSGALYESVNTNCVLASGA |
| | FHI_NMBU_10 | | | MIVSAFGGPIGLISTGLAAIGACATSSSSSSAS |
| | | | | ASAGNGS |
| EN34 | Enterobacter | A0A145H764 | 707. | MRELNESELSNVSGAGMWGSIGSAIGGMFG |
| | cloacae | | | GGSAGCNGSYSHTSTTNKDGSHSESTSFSGG |
| | EN3600 | | | CSVNVGGKSGGSSKGNGGSKSGGGRQGND |
| | | | | HGRH |
| EN359 | Raoultella | A0A3P8JDH9 | 708. | MRALTEVELSFIYGADGNDSYNPISLIVGSLS |
| | terrigena | | | TIAKLPTPVSLAVGIGTEIAIQASPHMPVHVP |
| | (Klebsiella | | | VPKIPMGPTWNGSGGGRPSPSASLSTPDSSN |
| | terrigena) | | | |
| | NCTC13098 | | | |
| EN42 | Salmonella | A0A619I0P2 | 709. | MRALTDNEVESVYGADRGDSAVTGAVAGG |
| | enterica | | | TAGGAAGGWAGAELGAELGSLAGPIGTFVG |
| | (Salmonella | | | FLGGAAIGAFGGAYIYDSFSSPSNGS |
| | choleraesuis) | | | |
| | PNUSAS101199 | | | |
| EN421 | Escherichia coli | A0A5R1MAD6 | 710. | MRTLTLNELDSVSGGASGRDIAMAIGTLSGQ |
| | 2274-3 | | | FVAGGIGAAAGGVAGGAIYDYASTHKPNPA |
| | | | | MSPSGLGGTIKQKPEGIPSEAWNYAAGRLCM |
| | | | | DLPLYFQTPVIT |
| EN43 | Salmonella | A0A1J6ZK55 | 711. | MRELNEHEIKCAAGGQDYGREIAQGAGAAA |
| | enterica subsp. | | | GTFVAGGGGAIVGPIVAGMVYDWASNKTPD |
| | houtenae | | | SALSPSGLGSTLKKKPEGLPSEAWNYAEGRM |
| | serovar | | | CNWSPNNLNDVCK |
| | 50:g,z51:-, | | | |
| | FDAARGOS | | | |
| | 55 | | | |
| EN44 | Salmonella | A0A379QD49 | 712. | MRELDNKELDFVGGAGDPLADPNSQILRQV |
| | enterica | | | MANAAWGAAFGIRGGLPGMAVGMAGGVA |
| | (Salmonella | | | QTVIQGAAAQMPVNVPIPQVPMGPTWNGSK |
| | choleraesuis) | | | G |
| | NCTC10252 | | | |
| EN49 | Salmonella | A0A3Z6QTT4 | 713. | MRNLTLNELKSISGAGDGDGVGRDIATALG |
| | enterica subsp. | | | GLAGGFVAGAVGAAAGGVAGGAVYDFAST |
| | enterica serovar | | | HTVNPAMSPSGLGGYLGGYYGSSSSAVNSG |
| | Java 412099 | | | S |
| EN51 | Enterobacter sp. | A0A3N1J5S6 | 714. | MRELSQQDLVLAFGAGDANSQLISDMGNNA |
| | BIGb0383 | | | AWGAGLGAITGGGPGAAIGAVGGVVQTIGQ |
| | | | | GLIDHGPVSVPVPVMIGPSWNGSGGWSGAG |
| | | | | FGSSSGGAGSAS |
| EN511 | Leclercia | A0A5B7XGC6 | 715. | MRKLNSKEMKLMGGAGDPLTDQNSQLIRQI |
| | adecarboxylata | | | MIGAGWGMMWGVPMGFPGMAAGAITATT |
| | Z96-1 | | | QTVLQGAMSRMPVNVLIPRVPMGPTWNGSK |
| EN52 | Escherichia coli | A0A3T3ZPP2 | 716. | MRNITTDELALIHGAGLDKDASVAIGTAAGG |
| | 857A | | | FLGKATKIPSADIAGSAIGGYIGGVIADGSNR |
| | | | | TITIPSNGINYNPSIGLGSFNPNYNGGLLNSGF |
| | | | | NSSTSSSSSGS |
| EN64 | Yokenella | A0A6H0K645 | 717. | MSNIRELSFDEIASVSGGEGHGSEVAQDRRD |
| | regensburgei | | | ARNSLGRNAPNKIYGGTEKCVNNILIGAGGG |
| | W13 | | | AVTGGLPGAAVGLVGGAYAGECLSGGGNG |
| | | | | NGNGCNAGSNNCSSGNAASTCNR |
| EN76 | Escherichia coli | A0A4T9HR51 | 718. | MREITLNEMNNVSGAGDVNWVDVGKTVAT |
| | FHI_NMBU_10 | | | NGAGVVGGAFGAGLCGPVCAGVFAVGSSA |
| | | | | AVGALYDAAGNSKPTKQKPEGLPPEAWNYA |
| | | | | EGRMCNWSPNNLNDVCL |
| EN77 | Salmonella | A0A702KVZ3 | 719. | MRELNEHEIKCAAGGQDYGREIAQGAGAAA |
| | enterica subsp. | | | GTFVAGGGGAIVGPIVAGMVYDWASNKTPD |
| | salamae 12- | | | SALSPSGLGSTLKQKPEGLPSEAWNYTEGRM |
| | 2127 | | | CNWSPNNLNDVCK |
| EN82 | Citrobacter | A0A2U4EKK8 | 720. | MRELSISEINFVSGGNANSNYENGRNTSSSRG |
| | amalonaticus | | | SNYNNQGRNNNSGFYGGLGQDVTNCNNGII |
| | FDAARGOS_1 | | | GGAIAGSLGGLAGVAVGITGGAFAGGCFSH |
| | 22 | | | GNGNGGGSGANSNCSSSGIGGTCK |
| ER14 | Pantoea | | 721. | MRELNSEEIRNVAGAGTDNPYDNPILLMNQI |
| | agglomerans | | | AENAAWGAALGAYGGPEGVILGAGGGVVQ |
| | FDAARGOS | | | TVVQGAAAQMPVNVPIPQVPLGPTWNGSGS |
| | 407 | | | N |
| ER18 | Mixta gaviniae | | 722. | MRMLINNDIKLVTGAGANTATEYVGHMKN |
| | DSM 22758 | | | ADKYAGGSGSGNKGGGFYTSQGVVDCNNGI |
| | | | | IGGVIMGAYGLGWGMALGVVGGALAGGCF |
| | | | | KTEGGNGAGGGSGSNGSNCNDGGCSW |
| ER31 | Pantoea allii | | 723. | MLRELTDSEINIVSGGNANSGYEGPNCTNAA |
| | PNA 200-10 | | | IKGGVTGATGGAVAGAIRGGVTGLAGGPAG |
| | | | | VVAGAGTGAAEGAVYGGVSGAVGGYVGCQ |
| | | | | NSGSANNGGNNGNLGGDSNANSANGQCHW |
| ER58 | Erwinia | | 724. | MENINTVLMEKVSGAGNYADKHEIGNSSGR |
| | mallotivora BT- | | | GGAPDSCANRAGLGAIIGTLGALATGATGGL |
| | MARDI | | | AGVVVAGVTGGLSSVIGCENPSYNDNNSGN |
| | | | | SSRSGNYGAQCTW |
| FF371 | Vibrio sp. | | 725. | MRELNNNEILIVDGGGERGGGDHSGEWSSW |
| | strain FF_371 | | | SGSYSNNSNSSNNSNYPASEQAVQQQANSPT |
| | | | | QDCAQAVAGNAIGGAIAGSGGGLAGAGLGA |
| | | | | LGGGLAGALGGSCNQ |
| HUW04 | Vibrio cholerae | | 726. | MKELSIEQISLVTGGERGDGRDRHSLLHELK |
| | 4772STDY6941189 | | | DIAQYLITRNPLGLITHSEPLNAGEEAWLKER |
| | | | | EREGARHGYNERQGGYHRGDGGRHIEN |
| RFB05 | Vibrio cholerae | | 727. | MKTLTLDEMKIVSGGRHAEGAYNANGPDRT |
| | strain RFB05 | | | REQARHENYPGREWSSADLRNAGLALSAVG |
| | | | | AVTPGVPGRVIGALGAGAALIGGWERNGGN |
| | | | | H |
| Splendid | Vibrio | | 728. | MRSLNASEVNMVSGGREARGAYNSNGPDRT |
| | splendidus | | | RAQAMRENNPGRDWSNSDLNNVSLALGLA |
| | 5S/ZS | | | GTLVPGIWGRGISALGAAGAAVSGWKRKDK |
| Vthal | Vibrio thalassae | | 729. | MYNLSADEISMVDGGGDGNSSGYPASPSAV |
| | strain | | | AQQANSPSRACGQAIATGAVVNGMAGGYM |
| | CECT8203 | | | GGLKGAVAGAV |

Examples of growth factors include, but are not limited to, erythropoietin, epidermal growth factor, platelet-derived growth factor, tumor necrosis factor, interleukins (e.g., IL-1, IL-2), insulin (including single-chain insulin), and the like.

Examples of pheromones include, but are not limited to, bacterial, fungal, arthropod, annelid, mollusk, and vertebrate pheromone peptides as described, for example, by Altstein (“Chapter 210—Pheromone Peptides” in Handbook of Biologically Active Peptides, Editor: Abba J. Kastin, Academic Press, 2006, pages 1505-1513), which is incorporated herein by reference in its entirety. Examples of peptide hormones include, but are not limited to, adrenocorticotropic hormone, amylin, angiotensin, atrial natriuretic peptide, calcitonin, cholecystokinin, gastrin, ghrelin, glucagon, growth hormone, follicle-stimulating hormone, insulin (including single-chain insulin), leptin, melanocyte-stimulating hormone, oxytocin, parathyroid hormone, prolactin, renin, somatostatin, thyroid-stimulating hormone, thyrotropin-releasing hormone, vasopressin, vasoactive intestinal peptide, and others as described, for example, by Clapp et al. (Physiol. Rev. 2009, 89:1177-1215).

Examples of neuropeptides include, but are not limited to, N-acetylaspartylglutamic acid, cholecycstokinin, conotoxins, dynorphin, α-endorphin, β-endorphin, γ-endorphin, enkephalin, galanin, grehlin, neuropeptide S, neuropeptide Y, neurotensin, orexin A, and the like. The heterologous peptide may be a cell-signaling peptide such as a chemokine, a quorum sensing peptide, or a fungal mating factor. It will be appreciated that the heterologous peptide may be considered to belong to more than one category (e.g., a heterologous peptide may be considered to be a pheromone, a neuropeptide, and/or cell-signaling peptide).

Examples of protease inhibitors include, but are not limited to, Kunitz-type protease inhibitors, Bowman-Birk protease inhibitors, aprotinin, cystatins, hirudin, eglin C, serpins, and others as described, for example, by Rawlings et al. (Biochem. J. (2004) 378, 705-716).

Examples of self-assembling peptides include oligo- and polypeptides that form hierarchical structures including α-helix coiled coils, β-sheets, β-hairpins, micellar cylinders, cyclic peptide nanotubes, and the like. Self-assembling peptide sequences include, but are not limited to those set forth in Table 3.

TABLE 3

| Shorthand | | SEQ ID |
|---|---|---|
| Name | Sequence | NO: |
| V6D | VVVVVVD | 1 |
| Q11 | QQKFQFQFEQQ | 2 |
| KA4K | KAAAAK | 3 |
| KA6K | KAAAAAAK | 4 |
| RA6R | RAAAAAAR | 5 |
| K24 | NH2-KLEALYVLGFFGFFTLGIMLSYIR- | 6 |
| | COOH | |
| Lysb-21 | AcNH-QATNRNTDGSTDYGILQINSR-NH2 | 7 |
| EAK16 | AcNH-RERERKRKRERERKRK-COONH2 | 8 |
| DN1 | AcNH-QQRFQWQFEQQ-NH2 | 9 |
| P11-4 | AcNH-QQRFEWEFEQQ-NH2 | 10 |
| RAD16-1 | AcNH-RADARADARADARADA-COONH2 | 11 |
| KLD12 | AcNH-KLDLKLDLKLDL-COONH2 | 12 |

Other self-assembling peptides are described, for example by Hosseinkhani, et al. (Chemical Reviews, 2013, 113, 4837-4861) and Lee, et al. (Int. J. Mol. Sci. 2019, 20, 5850), which are incorporated herein by reference in their entirety.

In some embodiments, the heterologous peptide is H. sapiens epidermal growth factor, an H. sapiens endorphin, S. cerevisiae α-factor, or H. medicinalis eglin C.

In some embodiments, the heterologous peptide is an antibody or fragment thereof. For example, and not to be limiting, in some embodiments, the antibody or fragment there is selected from the group consisting of a Fab fragment, a Fab′ fragment, a F(ab′) fragment, a Fv fragment, a diabody, a ScFv, a small modular immunopharmaceutical (SMIP), an affibody, an avimer, a nanobody, a domain antibody and/or single chains. In some embodiments, the antibody or fragment thereof is humanized. In some embodiments, the affibody is an anffibody set forth in Table 4.

TABLE 4
Exemplary Affibodies

| Names | SEQ ID NO: | Amino Acid Sequence |
|---|---|---|
| Anti-protein A | 730 | VDNKFNKETQEASWEIF |
| affibody ZpA | | TLPNLNGRQVAAFISSLL |
| | | DDPSQSANLLAEAKKLN |
| | | DAQAPK |
| Anti-taq | 731 | VDNKFNKEQQNAFYEIL |
| affibody ZTaq | | HLPNLNEVQVKAFIDSLR |
| | | DDPSQSANLLAEAKKLN |
| | | DAQAPK |
| Parental | 732 | VDNKFNKENIAAMTEIT |
| affibody | | RLPNLNPYQRAAFIWSL |
| sequence ZWT | | KDDPSQSANLLAEAKKL |
| | | NDAQAPK |
| Anti-TNF | 733 | VDNKFNKENIAAMTEIT |
| affibody | | RLPNLNPYQRAAFIWSL |
| ZTNFa | | KDDPSQSANLLAEAKKL |
| | | NDAQAPK |
| Anti-FcRN | 734 | VDAKYAKEWMRAAHEI |
| affibody | | RWLPNLTFDQRVAFIHK |
| ZFcRn | | LEDDPSQSSELLSEAKKL |
| | | NDSQAPK |
| Anti-TNF | 735 | VDNKFNKELGWAIGEIG |
| affibody 2 | | TLPNLNHQQFRAFILSLW |
| ZTNFa185 | | DDPSQSANLLAEAKKLN |
| | | DAQAPK |

### A. Secretion Signal Sequences

Microcin secretion signal sequences are particularly useful for directing secretion of peptides using the gram-negative bacterial cells of the present disclosure. For example, the signal sequence may be associated with secretion machinery used by microbes in the production of microcins including, but not limited to, MccL, MccV, MccS, MccE492, MccM, MccH47, MccPDI, MccN, MccI47, and MccG492.

In some embodiments, the secretion signal sequence is an N-terminal sequence:

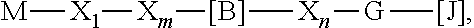

wherein:
M is methionine,
G is glycine,
each residue “X” is independently any amino acid,
subscript 1 is an integer ranging from 0 to 9, subscript m is 2, and subscript n is 9,
residue “B” is isoleucine or leucine, and
residue “J” is alanine or glycine.
   Residue “J” is fused to the heterologous peptide to be secreted directly or via a linker sequence (e.g., one or more glycine residues).

In some embodiments, the residue at position-14 with respect to the cleavage site is arginine (R), lysine (K), or glutamic acid (E). In the residues at −4 and −7 with respect to the cleavage site are independently alanine (A), isoleucine (I), leucine (L), methionine (M), phenylalanine (F), or valine (V).

In some embodiments, the secretion signal sequence is an N-terminal sequence:

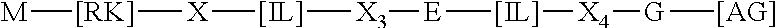

wherein:
M is methionine,
G is glycine,
[RK] is arginine or lysine,
[IL] is isoleucine or leucine,
[E] is glutamic acid,
[AG] is alanine or glycine, and
each residue “X” is independently any amino acid.

In some embodiments, the N-terminal sequence comprises an amino acid sequence as set forth in Table5.

TABLE 5

| | SEQ | |
|---|---|---|
| | ID | |
| Taxon | NO | Sequence |
| Carnobacterium | 13 | MKTVKELSVKEMQLTTGG |
| maltaromaticum | | |
| Carnobacterium | 14 | MNSVKELNVKEMKQLHGG |
| maltaromaticum | | |
| Carnobacterium | 15 | MKSVKELNKKEMQQINGG |
| maltaromaticum | | |
| Enterococcus faecium | 16 | MKHLKILSIKETQLIYGG |
| Enterococcus faecium | 17 | MEKLTVKEMSQVVGG |
| Escherichia coli | 18 | MRTLTLNELDSVSGG |
| Escherichia coli | 19 | MREITLNEMNNVSGA |
| Escherichia coli | 20 | MRELDREELNCVGGA |
| Escherichia coli | 21 | MANIRELTLDEITLVSGG |
| Escherichia coli | 22 | MSNIRELSFDEIALVSGG |
| Escherichia coli | 23 | MREITESQLRYISGA |
| Escherichia coli | 24 | MREISDNMLDSVKGG |
| Escherichia coli | 25 | MRKLSENEIKQISGG |
| Klebsiella | 26 | MREISQKDLNLAFGA |
| pneumoniae | | |
| Klebsiella | 27 | MRALTENDFFAVSGA |
| pneumoniae | | |
| Lactobacillus | 28 | MNNVKELSMTELQTITGG |
| curvatus | | |
| Lactobacillus sakei | 29 | MNNVKELSMTELQTITGG |
| Lactobacillus sakei | 30 | MEKFIELSLKEVTAITGG |
| Leuconostoc carnosum | 31 | MNNMKSADNYQQLDNNALEQVVGG |
| Leuconostoc gelidum | 32 | MMNMKPTESYEQLDNSALEQVVGG |
| Leuconostoc | 33 | MTNMKSVEAYQQLDNQNLKKVVGG |
| mesenteroides | | |
| Mixta gaviniae | 34 | MRMLINNDIKLVTGA |
| Pantoea agglomerans | 35 | MRELNSEEIRNVAGA |
| Pantoea allii | 36 | MLRELTDSEINIVSGG |
| Pediococcus | 37 | MKKIEKLTEKEMANIIGG |
| acidilactici | | |
| Streptococcus uberis | 38 | MNTIEKFENIKLFSLKKIIGG |
| Synechococcus | 39 | MSQFINLINDQNAQELLGG |
| Synechococcus | 40 | MSKLINTIDDNHAQSLVGG |
| Synechococcus | 41 | MNQWLESINDDTAQQLLGG |
| Synechococcus | 42 | MQVEFDLITDLSDGQAEMVTGA |
| Vibrio cholerae | 43 | MKELSIEQISLVTGG |

### B. PCATs

Gram-negative bacterial cells for peptide secretion according to the present disclosure contain nucleic acids encoding C39 peptidase-containing ATP-binding cassette (ABC) transporters (PCATS), such as Escherichia coli CvaB (UniProt Accession #P22520). PCATs are characterized by three domains: a C39 peptidase domain, a transmembrane domain (TMD) and a nucleotide binding domain (NBD). PCATs typically form a homodimer to function, cleaving leader peptides from various bacteriocin precursor peptides. The C39 peptidase domain, which is at N-terminus, is characterized by protease activity and catalytic residues Cys32, His105, Asp121 (CvaB numbering). The PCAT TMD generally consists of six hydrophobic alpha-helices, anchoring the PCAT in the bacterial inner membrane. The cytoplasmic NBD, which is at C-terminus, is the site of ATP binding and hydrolysis.

In some embodiments, the PCAT is E coli CvaB, a Streptococcus ComC, or C. thermocellum PCAT1. In some embodiments, the PCAT comprises an amino acid sequence as set forth in Table 6.

TABLE 6

| | | | | SEQ | |
|---|---|---|---|---|---|
| | ID/RefSeq | | | ID | |
| Group | Accession | Description | Taxon | NO | Sequence |
| 1 | E492_PCAT_mceG | peptidase | Klebsiella | 44 | MSNGNVRRMINQLDMRWRRRVPVIH |
| | | domain- | pneumoniae | | QTETSECGLACLAMICGHFGKNIDL |
| | | containing | | | ISLRRKFNLSARGANLAGINSIAEQ |
| | | ABC | | | LGMVTRALSLELDELGALKTPCILH |
| | | transporter | | | WDFSHFVVLVSVKRNRYVLHDPARG |
| | | | | | RRNVGLEEMTRYFTGVALEVWPGSE |
| | | | | | FSEETTQNRIQLRSLINSIYGIKST |
| | | | | | LAKIFCLSVVIEAINLVMPVGTQLV |
| | | | | | MDHAIPAGDRGLLTLISAGLMFFIL |
| | | | | | LRAATGMLRAWSSLVMGTLINVQWQ |
| | | | | | SGLFNHLLRLPLAYFERRKLGDIQS |
| | | | | | RFGSLDTLRATFTTSVVGAIMDSIM |
| | | | | | VVGVFVMMLLYGGYLTWIVLGFTTV |
| | | | | | YVLIRLVTYGYYRQISEETLVRGAR |
| | | | | | ASSYFMETLYGIATVKIQGMAERRG |
| | | | | | THWLNLKIDAINSGIKLTKMDLFFG |
| | | | | | GINTFVAACDQVAILWLGTSLVIDN |
| | | | | | QMTIGMFVAFGSFRGQFSDRVASLT |
| | | | | | NFLLQLRMMSLHNERIADIALHEKE |
| | | | | | EKKPELDIVADMTPVSLETTDLSYR |
| | | | | | YDSQSAPVFSGLYLSVTPGESVAIT |
| | | | | | GTSGAGKTTLMKVLCGLFEPDSGKV |
| | | | | | LVNGTDIRQLGINNYHRMIACVMQD |
| | | | | | DRLFSGSIRENICGFSEEMDEAWMI |
| | | | | | ECARASYIHDVIMKMPMGYETLIGE |
| | | | | | LGEGLSGGQKQRIFIARALYRRPGI |
| | | | | | LFMDEATSSLDTESERFVNVAIRNM |
| | | | | | NITRVIIAHRETTLRTVDRTISI |
| 1 | WP_024913114.1 | peptidase | Chania | 45 | MNKLSFKCLIKQLDIRLFQRIPLVH |
| | | domain- | multitudinisentens | | QTEASECGLACLAMICGHYGKNIDL |
| | | containing | | | IALRQQFNLAARGTTLASLTGIADQ |
| | | ABC | | | LGLATRPLSLDLNELSALKKPCILH |
| | | transporter | | | WEFNHFVVLVSVGRHRVVLHDPARG |
| | | | | | RRTVSLSELSSSFTGVALEVWPGGA |
| | | | | | FTADTIRDRFSFGTLIGSVHGLKGT |
| | | | | | LGKIFCLSLVIETINLVMPVGTQLV |
| | | | | | MDHAIPAGDRGLLTLICAGLMLFIL |
| | | | | | LRTAVSMVRAWSSLVMSTLINVQWQ |
| | | | | | SGLFNHLLRLPLGYFERRKLGDIQS |
| | | | | | RFGSLDTLRQTFTTSIVGAIMDGIM |
| | | | | | IMGVLVMMVLYGGWLTWVVVAFTTL |
| | | | | | YVLLRLITYVYYRQLSEESLIRKAR |
| | | | | | AGSYFMETLYGIATVKMQGMTERRS |
| | | | | | AHWLNLEIDAINTGIRVTKMDMLFG |
| | | | | | GINTFVAACDQIVILWLGTRLVIDT |
| | | | | | QMTIGMFVAFAAFRGQFSDRISSLT |
| | | | | | EFLLQLRLMSLHNERIADIALHPRE |
| | | | | | NRQPAIVYEPQLRPATLTTQALSYS |
| | | | | | YDSQSPPIFNKLDIVVAPGESVAIV |
| | | | | | GPSGAGKTTLMKVLCGLFLPDHGKV |
| | | | | | EINGVDIQQLGINNYHKMIACVMQD |
| | | | | | DKLFSGSIRENISGFSDDVDEDWMQ |
| | | | | | ECARASYLHDVIMRMPMRYETLIGE |
| | | | | | LGEGLSGGQKQRLFIARAIYKKPTI |
| | | | | | LFLDEATSALDKESETTVNQSIKAL |
| | | | | | RITRIIIAHRESTIASADRVITLNP |
| | | | | | QL |
| 1 | WP_007375113.1 | MULTISPECIES: | Kosakonia | 46 | MSNKSLFTALLGKLDLSWFRRVPMV |
| | | peptidase | | | HQTESSECGLASLAMICGHYGKNID |
| | | domain- | | | LISLRQQFNLSARGTTLIGLNEIAG |
| | | containing | | | QLGLATRALSLDLHELKLLKTPCVL |
| | | ABC | | | HWDFNHFVVLVAVKGNKYVINDPAR |
| | | transporter | | | GRRTVGQAEMSQYFTGVALEAMPGS |
| | | | | | EFTQEKVKNRVRLRSLIGSVHGIKA |
| | | | | | TLAKIFCLSIVIEAINLVLPVGTQL |
| | | | | | VMDHAIPAGDRGLLSLICIGLFFFI |
| | | | | | LLRASVSMLRAWTSLVMSTLINVQW |
| | | | | | QSGLFRHLIKLPLGYFERRKMGDIQ |
| | | | | | SRFGSLDTLRETFTSSVVGAIMDGI |
| | | | | | MVVGVLIMLVLYGGWLTWVVLGFTA |
| | | | | | VYVLIRLLTYNYYRQLSEERLVRGA |
| | | | | | RASSYFMETLYGIATVKMQDMSERR |
| | | | | | GTHWLNLQVDTINTGIKLTRLDLFF |
| | | | | | NGLNTFIAACDQVAILWLGTSLVID |
| | | | | | NQMTIGMFVAFGAFRGQFSDRIGSL |
| | | | | | VDFLLQLRMMSLHNERISDIALHER |
| | | | | | EPRKPDLPATTEMRPLGLETTGLSY |
| | | | | | RYDSQSAPIFNDLALRIEPGESVAL |
| | | | | | VGASGAGKTTLMKVLCGLFEPDAGK |
| | | | | | VLVDGMDIKQLGINNYHKMIGCVLQ |
| | | | | | DDKLFAGSLRENICGFAEHADEEWM |
| | | | | | VECAKASHIHDVILAMPMGYETLIG |
| | | | | | ELGEGLSGGQKQRVFIARALYRKPG |
| | | | | | LLFMDEATSALDHESEACVNNAIKQ |
| | | | | | LNITRVIIAHRATTIATADRVINL |
| 1 | WP_064566347.1 | peptidase | Kosakonia | 47 | MSNKSLFSALLGKLDLSWFRRVPMV |
| | | domain- | oryzae | | HQTESSECGLASLAMICGHYGKNID |
| | | containing | | | LISLRQQFNLSARGTTLIGLNEIAG |
| | | ABC | | | QLGLATRALSLDMHELKLLKTPCVL |
| | | transporter | | | HWDFNHFVVLVAVKGNKYVINDPAR |
| | | | | | GRRTVGQAEMSQYFTGVALEAMPGS |
| | | | | | EFTQEKVKNRVRLRSLVGSVHGIKA |
| | | | | | TLAKIFCLSIVIEAINLVLPVGTQL |
| | | | | | VMDHAIPAGDRGLLSLICIGLFFFI |
| | | | | | MLRASVSMLRAWTSLVMSTLINVQW |
| | | | | | QSGLFRHLIKLPLGYFERRKMGDIQ |
| | | | | | SRFGSLDTLRETFTSSVVGAIMDGI |
| | | | | | MVVGVLIMLVLYGGWLTWVVLGFTA |
| | | | | | VYVLIRLLTYNYYRQLSEERLVRGA |
| | | | | | RASSYFMETLYGIATVKMQDMSERR |
| | | | | | GTHWLNLQVDTINTGIKLTRLDFFF |
| | | | | | NGLNTFIAACDQVAILWLGTSLVID |
| | | | | | NQMTIGMFVAFGAFRGQFSDRVGSL |
| | | | | | VEFLLQLRMMSLHNERISDIALHER |
| | | | | | EPRKPDLPATTEMRPLGLETTGLSY |
| | | | | | RYDSQSAPIFNDLALRIEPGESVAL |
| | | | | | VGASGAGKTTLMKVLCGLFEPDAGK |
| | | | | | VLVDGMDIKQLGINNYHKMIGCVLQ |
| | | | | | DDKLFAGSLRENICGFAEHADEEWM |
| | | | | | VKCAKASHIHDVILAMPMGYETLIG |
| | | | | | ELGEGLSGGQKQRVFIARALYRKPG |
| | | | | | LLFMDEATSALDHESEACVNNAIKQ |
| | | | | | LNITRVIIAHRATTIATADRVINL |
| 1 | WP_108901370.1 | peptidase | Limnobaculum | 48 | MDKATFTHLLESLNFGMRKKVPQIL |
| | | domain- | parvum | | QTEAAECGLACLAMISRYYGMNVDL |
| | | containing | | | FNLRRRFGISSHGATLGVVINTASE |
| | | ABC | | | INLKSRALTLDLNEINQLKTPCILH |
| | | transporter | | | WDLNHFVVLVEVKRNKFVLHDPAFG |
| | | | | | RRVVSEQEMSQHFTGVALEVWPGNE |
| | | | | | FVPYNETSRLSLWSMMSHIEGLKGF |
| | | | | | LVKVFALSIVIETVNLLIPVGTQLV |
| | | | | | MDHVILAEDHDLLGLICLGLLFFIL |
| | | | | | FRTFVGMLRSWTSLVMEALVGVQWK |
| | | | | | AGLFDHLMKLPLSYFEKRKLGDIQS |
| | | | | | RFISLDTIRTTLTSNLVSSIIDSLM |
| | | | | | AIGVFIMMLLYGGWLVWVVLGFTVL |
| | | | | | YMILRLSTYQHYRQASEEQIIKGAK |
| | | | | | ANSHFMETLYGISTLKALGLTATRG |
| | | | | | QYWLNLNIDTTNANIRLTKLDMVFG |
| | | | | | GMNTLLGMVDQIVILWLGASMVIDG |
| | | | | | HMTLGMFVAFNAYRGQFSERSANLI |
| | | | | | NLALELKMLSLHSERLTDIVFSEPE |
| | | | | | NTLPNNMVFPEDQPLGFEVRHVEFR |
| | | | | | YDNLSAPIFSNLNLKIEPGENVAII |
| | | | | | GPSGIGKTTLMKVMAGLLLPSQGEV |
| | | | | | LANGIDINKIGVNNYRRYIACVLQD |
| | | | | | DKLFAGSIADNIASFEAQKDSAWVV |
| | | | | | ECAKHCNIHEEIMRMPMGYETLITE |
| | | | | | LGGSLSGGQKQRLLIARALYRRPSL |
| | | | | | LFLDEATSHLDTANEAHINAAISAL |
| | | | | | KVTRIFIAHRLSTIESADRVINLDP |
| | | | | | VEEFVAVTPD |
| 1 | WP_013576039.1 | MULTISPECIES: | Rahnella | 49 | MNLDNVRELADRLHFSWRHRVPQII |
| | | peptidase | | | QTEAAECGLASLAMVFGYHGKHVDL |
| | | domain- | | | QSLRQRYGISSRGATLRTLMDIAAA |
| | | containing | | | NEMKSRALKLDIDELNALKTPCILH |
| | | ABC | | | WDLNHFVVLVGVKQGKVIIHDPAFG |
| | | transporter | | | RRSLGMREVSEHFTGVALELWPDNG |
| | | | | | FTSEKPRVRLDLRKMMGNVSGLIPA |
| | | | | | LTKIFCLSLMIESVNLLLPVGMQLV |
| | | | | | MDHVIPAKDPDLLTLICLGLLFFII |
| | | | | | FRTGVSMLRAWTSLVMSTLIDVQWK |
| | | | | | ARLFDHLMKLPLDYFEKRKLGDIQS |
| | | | | | RFTSLDTLRTTLTTNVVNSIIDGIM |
| | | | | | SVGLIVMMVLYGGWLIWVILGFTAI |
| | | | | | YVIFRLTTYNAYRQVSEEQIVKGAR |
| | | | | | AGSHFMETLYGISTLKALGLSKARA |
| | | | | | NFWLNLNIDNANATVRKTRFEMMFT |
| | | | | | GGNTLIATLDQVALLWLGATQVIDG |
| | | | | | HMTLGMFVAFNTYRGQFSERAASLL |
| | | | | | NMVLQLRMLSLHRDRIADIALTGTE |
| | | | | | KSLPERELLRAGEAASLEVRDLVFQ |
| | | | | | YDSLSAPLINGLNLSVAAGESVAIT |
| | | | | | GPSGQGKTTLMKIMTGLLTPTEGRI |
| | | | | | LINGMDITTAGLNNYRSCIACVLQD |
| | | | | | DTLFAGSIAENIASFDEQKDEARII |
| | | | | | DCARRCNIHDDIERMPMGYETLISE |
| | | | | | LGGSLSGGQKQRLLIARALYRRPAI |
| | | | | | LFLDEATSHLDLDNEMRINQAISEL |
| | | | | | DITRIFIAHRPSTIASADREIKLG |
| 1 | WP_112152189.1 | MULTISPECIES: | Rahnella | 50 | MNLDNVRELADRLHFSWRHRVPQII |
| | | peptidase | | | QTEAAECGLASLAMVFGYHGKHVDL |
| | | domain- | | | QSLRQRYGISSRGATLRTLMDIAAA |
| | | containing | | | NEMKSRALKLDIDELNALKTPCILH |
| | | ABC | | | WDLNHFVVLVGVKQGKVIIHDPAFG |
| | | transporter | | | RRSLGMREVSEHFTGVALELWPDNG |
| | | | | | FTSEKPRVRLDLRKMMGNVSGLIPA |
| | | | | | LTKIFCLSLMIESVNLLLPVGMQLV |
| | | | | | MDHVIPAKDPDLLTLICLGLLFFII |
| | | | | | FRTGVSMLRAWTSLVMSTLIDVQWK |
| | | | | | ARLFDHLMKLPLDYFEKRKLGDIQS |
| | | | | | RFTSLDTLRTTLTTNVVNSIIDGIM |
| | | | | | SVGLIVMMVLYGGWLIWVILGFTAI |
| | | | | | YVIFRLTTYNAYRQVSEEQIVKGAR |
| | | | | | AGSHFMETLYGISTLKALGLSKARA |
| | | | | | NFWLNLNIDNANATVRKTRFEMMFT |
| | | | | | GGNTLIATLDQVALLWLGATQVIDG |
| | | | | | HMTLGMFVAFNTYRGQFSERAASLL |
| | | | | | NMVLQLRMLSLHRDRIADIALTDTE |
| | | | | | KSLPERELLRAGEAASLEVRDLVFQ |
| | | | | | YDSLSAPLINGLNLSVAAGESVAIT |
| | | | | | GPSGQGKTTLMKIMTGLLTPTEGRI |
| | | | | | LINGMDITTAGLNNYRSCIACVLQD |
| | | | | | DTLFAGSIAENIASFDEQKDEARII |
| | | | | | DCARRCNIHDDIERMPMGYETLISE |
| | | | | | LGGSLSGGQKQRLLIARALYRRPAI |
| | | | | | LFLDEATSHLDLDNEMRINQAISEL |
| | | | | | DITRIFIAHRPSTIASADREIKLG |
| 1 | WP_032716442.1 | MULTISPECIES: | Raoultella | 51 | MNKDVLDSIFKRINFSMKNKVPVII |
| | | peptidase | | | QSESTECGNACLSMICGFYGKDIDL |
| | | domain- | | | FNFRNRYGSTSQGATLNVLAAIAQK |
| | | containing | | | AGLKTRALSLDIAEIKELRLPCILH |
| | | ABC | | | WSLNHFVVLVAIKGKRFIIHDPALG |
| | | transporter | | | RRVVHLQELSENFSGIALEAWPDSD |
| | | | | | FRQEKQRSRLKLLDLMHNMVGLKSA |
| | | | | | LIKIFMLSVVIETVNLLLPMGTQIV |
| | | | | | TDHVITAHDENLLLVICVGLMFFTI |
| | | | | | FKTWVSMIRAWVSLKLNTLTDVQWK |
| | | | | | TSFFDHLMSLPLAFFEKRQLGDIQS |
| | | | | | RFASLDIIRATFTNSIVTGMIDSIM |
| | | | | | TIGLLIMLSLYGGWLVWVVLGFTVC |
| | | | | | YAIMRALTYKFYRTVSEELIVKRAR |
| | | | | | SGSHFMESLYGIATIKSLNLKNRRS |
| | | | | | QHWLNTNIDVSNAGLKQTRFDMLFG |
| | | | | | GINTFINSADQVVILWLGAQMVMDN |
| | | | | | TMTIGMFMAFNAYRGQFTQRAASLI |
| | | | | | DLTMQLKMLSLHNERISEIVYSEPE |
| | | | | | VDSPLRNVFEENVGVSLEVRDLAYQ |
| | | | | | YDLLSKPVFSNVNISVAAGESVALV |
| | | | | | GVSGIGKTTLLKVMSGLLTPERGEI |
| | | | | | FIGGFDINKIGINNFRSNIACVLQE |
| | | | | | DRLFSGSITDNISGFDDEVDEALVI |
| | | | | | ECAMQCNIHEEILRMPMGYETIIGE |
| | | | | | LGAGISGGQKQRLLIARALYQKPRI |
| | | | | | LFMDEATSHLDINNEKIINAAIESL |
| | | | | | NITRIIIAHRPSTIACADRIIDLAK |
| | | | | | ITP |
| 1 | WP_001838495.1 | peptidase | Salmonella | 52 | MQQGRSMSNGNVRRMINQLDMRWRR |
| | | domain- | enterica | | RVPVIHQTETSECGLACLAMICGHF |
| | | containing | | | GKNIDLISLRRKFNLSSRGANLAGI |
| | | ABC | | | NSIAEQLGMVTRALSLELDELGALK |
| | | transporter | | | TPCILHWDFSHFVVLVSVKRNRYVL |
| | | | | | HDPARGRRNVGLEEMTRYFTGVALE |
| | | | | | VWPGSEFSAETTQNRIHLRSLINSI |
| | | | | | YGIKSTLAKIFCLSVVIEAINLIMP |
| | | | | | VGTQLVMDHAIPAGDRGLLTLISAG |
| | | | | | LMFFILLRAATGMLRAWSSLVMGTL |
| | | | | | INVQWQSGLFNHLLRLPLAYFERRK |
| | | | | | LGDIQSRFGSLDTLRATFTTSVVGA |
| | | | | | IMDSIMVVGVFVMMLLYGGYLTWIV |
| | | | | | LGFTTVYVLVRLVTYSYYRQISEET |
| | | | | | LVRGARASSYFMETLYGIATVKIQG |
| | | | | | MAERRGTHWLNLKIDAINSGIKLTK |
| | | | | | MDLFFGGINTFVAACDQVAILWLGT |
| | | | | | SLVIDNQMTIGMFVAFGSFRGQFSD |
| | | | | | RVASLTNFLLQLRMVSLHNERIADI |
| | | | | | ALHEKEEKKPELDIVADMSPVSLET |
| | | | | | TDLSYRYDSQSAPVFSGLYLSVTPG |
| | | | | | ESVAITGTSGAGKTTLMKVLCGLFE |
| | | | | | PDTGKVLVNGTDIRQLGINNYHRMI |
| | | | | | ACVMQDDRLFSGSIRENICGFSEEM |
| | | | | | DETWMIECARASYIHDVIIKMPMGY |
| | | | | | ETLIGELGEGLSGGQKQRIFIARAL |
| | | | | | YRRPCILFMDEATSSLDTDSERFVN |
| | | | | | VAIKNMNITRIIIAHRETTLRTVDR |
| | | | | | VILI |
| 1 | WP_114159175.1 | peptidase | Salmonella | 53 | MQQGRSMSNGNIRRMINQLDMRWRG |
| | | domain- | enterica | | RVPVIHQTETSECGLACLAMICGHF |
| | | containing | | | GKNIDLISLRRKFNLSSRGANLAGI |
| | | ABC | | | NSIAEQLGMVTRALSLELDELGALK |
| | | transporter | | | TPCILHWDFSHFVVLVSVKRNRYVL |
| | | | | | HDPARGRRNVGLEEMTRYFTGVALE |
| | | | | | VWPGSEFSAETTQNRIHLRSLINSI |
| | | | | | YGIKSTLAKIFCLSVVIEAINLIMP |
| | | | | | VGTQLVMDHAIPAGDRGLLTLISAG |
| | | | | | LMFFILLRAATGMLRAWSSLVMGTL |
| | | | | | INVQWQSGLFNHLLRLPLAYFERRK |
| | | | | | LGDIQSRFGSLDTLRATFTTCVVGA |
| | | | | | IMDSIMVVGVFVMMLLYGGYLTWIV |
| | | | | | LGFTTAYVLIRLVTYSYYRQISEET |
| | | | | | LVRGARASSYFMETLYGIATVKIQG |
| | | | | | MAERRGTHWLNLKIDAINSGIKLTK |
| | | | | | MDLFFGGINTFVAACDQVAILWLGT |
| | | | | | SLVIDNQMTIGMFVAFGSFRGQFSD |
| | | | | | RVASLTNNLLQLRMVSLHNERIADI |
| | | | | | ALHEKEEKKPELDIVADMSPVSLET |
| | | | | | TDLSYRYDNQSAPVFSGLYLSVTPG |
| | | | | | ESVAITGTSGAGKTTLMKVLCGLFE |
| | | | | | PDTGKVLVNGTDIRQLGINNYHRMI |
| | | | | | ACVMQDDRLFSGSIRENICGFSEEM |
| | | | | | DETWMIECARASYIHDVIIKMPMGY |
| | | | | | ETLIGELGEGLSGGQKQRIFIARAL |
| | | | | | YRRPCILFMDEATSSLDTDSERFVN |
| | | | | | VAIKNMNITRIIIAHRETTLRTVDR |
| | | | | | VILI |
| 1 | WP_019453450.1 | MULTISPECIES: | Serratia | 54 | MKDHNLFERINEKLIFSLRKRVPSI |
| | | peptidase | | | LQSESSECGLACLAMISSYYGFNVD |
| | | domain- | | | MLSLRQRFGISTQGATLGTISQIAS |
| | | containing | | | QIQLKTRALSLDIDEVNQLKTPCVL |
| | | ABC | | | HWNMNHFVVLVKVRRASFVIHDPAF |
| | | transporter | | | GRRVIGLQEMSNHFTGIALELWPDR |
| | | | | | AFQKETLKTRLRLLDLMKNIEGLPG |
| | | | | | TLLKIFALSIVIESVNLLLPVGTQL |
| | | | | | VTDHVIQAHDYSLLTVICLGLIFFT |
| | | | | | LFRAVVSIARAWISIVLGTLTDIQW |
| | | | | | KTTLFEHLLKLPLDFFEKRHLGDIQ |
| | | | | | SRFSSLDAIRTTFTNNIVSGIIDGI |
| | | | | | MTVGLFVMMMVYGGWLVWVVSGFTL |
| | | | | | VYILIRMMTYRTYRQFSEEQIVKAA |
| | | | | | KANSHFMETLYGVSTVKALGIKDTR |
| | | | | | SSYWLNLNVDAANTNIKITRFNMMF |
| | | | | | GGINTFITTLDQVAILWLGAMMVID |
| | | | | | NSMTLGMFMAFNAYRGQFSQRASNL |
| | | | | | IDLAIGLRMLSLHNERISDIVFTDA |
| | | | | | EPESAPRQIFQPGKGVAVEVKNLTY |
| | | | | | QYDALSRPIFKDLDMCIAAGESVAV |
| | | | | | VGVSGAGKTTLLKVMCGLLSPTSGQ |
| | | | | | ILADSMDIHKVGVNNYRNAIACVLQ |
| | | | | | DDRLFSGSIAENISGFEVNADKALI |
| | | | | | VACAVHSNIHDEIMQMPMGYETLIG |
| | | | | | ELGNGISGGQKQRLFIARALYRRPS |
| | | | | | VLFMDEATSHLDVENELAINRAISS |
| | | | | | LNITRVIVAHRKSTIDSADRVVVLG |
| | | | | | AASGDPASNG |
| 1 | WP_021505808.1 | MULTISPECIES: | Serratia | 55 | MKDNNLFERINEKLTFSLRKRVPSI |
| | | peptidase | | | LQSESSECGLACLAMIASYYGFNVD |
| | | domain- | | | MLSLRQRFGISTQGATLGTISQIAS |
| | | containing | | | QIQLKTRALSLDIDEINQLKTPCIL |
| | | ABC | | | HWNMNHFVVLVKVQRAGFVIHDPAF |
| | | transporter | | | GRRVIGLQEMSNHFTGIALELWPDR |
| | | | | | AFQKETLKTRLRLLDLMKNIEGLPG |
| | | | | | TLLKIFALSIVIESVNLLLPVGTQL |
| | | | | | VTDHVIQAHDYSLLTVICLGLIFFT |
| | | | | | LFRAVVSIARAWISIVLGTLTDIQW |
| | | | | | KTTLFEHLMKLPLDFFEKRHLGDIQ |
| | | | | | SRFSSLDAIRTTFTNNIVSGIIDGI |
| | | | | | MTVGLFAMMMVYGGWLVWVVAGFTL |
| | | | | | IYILIRMMTYRTYRQFSEEQIVKAA |
| | | | | | KANSHFMETLYGISTVKALGIKETR |
| | | | | | SSYWLNLNVDAANTNIKITRFNMMF |
| | | | | | GGINTFITTLDQVAILWLGAMMVID |
| | | | | | NSMTLGMFMAFNAYRGQFSQRASSL |
| | | | | | IDLAIGLRMLSLHNERISDIVFTDA |
| | | | | | ETESAPRQVFPSGTGIAIEVKNLTY |
| | | | | | QYDALSRPIFKDLNMRIAAGESVAV |
| | | | | | VGASGAGKTTLLKVMCGLLSPTSGQ |
| | | | | | VLADAMDIHKVGVNNYRNAIACVLQ |
| | | | | | DDRLFSGSIAENISGFEVNANKELI |
| | | | | | MACAIHSNIHDEIMQMPMGYETLIG |
| | | | | | ELGNGISGGQKQRLFIARALYRRPS |
| | | | | | VLFMDEATSHLDVENESAINRAISS |
| | | | | | LNITRVIVAHRKSTIDSADRVVVLG |
| | | | | | AGSGAPAGGGE |
| 1 | WP_033633128.1 | MULTISPECIES: | Serratia | 56 | MTESYLDALKGKLNLSLRRKVPQIL |
| | | peptidase | | | QTEASECGLASLAMVCHYHGLQIDL |
| | | domain- | | | FNLRSRYGFSSRGATLSALIDIASA |
| | | containing | | | LKLQSRALSLNIDELKALKMPCILH |
| | | ABC | | | WDMKHFVVLVSVSRGRAVIHDPAFG |
| | | transporter | | | RKVLGLHELSRHFTGVALELWPDSE |
| | | | | | FQPIKQQSRLRFRKLMSNVRGLKGA |
| | | | | | LLKIFCLSIVIEAVNLLLPVGTQLV |
| | | | | | MDHVILAGDHDLLALICIGLLFFIL |
| | | | | | FRTGVSMLRSWISIVMGALIDVQWK |
| | | | | | AGVFDHLMKLPLSFFEKRKLGDIQS |
| | | | | | RFGSLDTIRATFTTSIVSSIIDGIM |
| | | | | | SVGVFIMMLLYGGWLVWVVLGFTAI |
| | | | | | YVILRLSTYQHYRQLSEEQLVKGAR |
| | | | | | AGSHFMETLYSVSTLKALGLSETRS |
| | | | | | QYWLNLNVETINAGIKLTKLNMMFG |
| | | | | | GLGAFIATCDQVIILWLGAALVIDN |
| | | | | | QMTIGMFIAFNAYRGQFSERASSLI |
| | | | | | DMMLQLRMLSLHNERVADIVLSSPE |
| | | | | | PQMPARQLFTKGNAAELQVRNLRYQ |
| | | | | | YDRLAKPIIADLNLSIAAGESVAIV |
| | | | | | GPSGVGKTTLMKLMCGLLSPDEGAV |
| | | | | | LVDGMDISNIGVNNYRQCIACVLQD |
| | | | | | DKLLAGSIAENISGFDAEMDLKRIE |
| | | | | | ACAQRCNMHDDIQNMPMGYETLIGE |
| | | | | | LGGSLSGGQKQRLLIARALYRRPSI |
| | | | | | LFMDEATSHLDLDNETHINRAISQL |
| | | | | | NITRVIIAHRPSTIASADRVIRLG |
| 1 | WP_047567592.1 | peptidase | Serratia | 57 | MSKPTFKQRISQLDLRLRHRVPMVH |
| | | domain- | marcescens | | QTESSECGLACLAMICGFYGKNVDL |
| | | containing | | | IALRRQFSLSTRGATLTGLTGIAEQ |
| | | ABC | | | LGLATRPLSLDIDELSALKLPCILH |
| | | transporter | | | WEFNHFVVLVSIKGHRAVLHDPARG |
| | | | | | RRAISLTELSHSFTGVALEAWPGSA |
| | | | | | FTADSVRHRIHLRTLIGSVHGLKGA |
| | | | | | LGKIFCLSLVFETINLVMPIGTQLV |
| | | | | | MDHAIPAGDRGLLTLICVGLMLFIL |
| | | | | | LRAAVGMVRAWSGLVMSTLINVQWQ |
| | | | | | SGLFTHLLRLPLGYFERRKLGDIQS |
| | | | | | RFGSLDTLRSTFTTSIVGAIMDGIM |
| | | | | | VIGVLVMMVLYGGWLTWVVIAFTAI |
| | | | | | YVLMRLLTYGYYRQLSEEALVREAR |
| | | | | | AGSYFMETLYGIATVKMQGMAERRI |
| | | | | | AHWLNLEIDTINTGIRVTRMDMLFG |
| | | | | | GINTFIAACDQVVILWLGASLVIDN |
| | | | | | QMTLGMFVAFGAFRGQFSDRIGSLT |
| | | | | | EFLLQLRMMSLHNERIADIALHPRE |
| | | | | | NRKPDLPYAAGLQPLGLSTHALSYR |
| | | | | | YDSQSPAVFDALDISVAPGESVAIV |
| | | | | | GPSGAGKTTLMKVLCGLFPPDSGRV |
| | | | | | EVNGVDIRQLGINNYHKMIACVMQD |
| | | | | | DKLFSGSIRENICGFADEVDDAWME |
| | | | | | ACARASYLHDVLMRMPMGYETPIGE |
| | | | | | LGEGLSGGQKQRLFIARAIYKKPAI |
| | | | | | LFLDEATSALDKESEAVVNQAIKRL |
| | | | | | NITKVIIAHRETTIASADRVIHFG |
| 1 | WP_060559630.1 | peptidase | Serratia | 58 | MSRSTFKQRVSQLDLRLRHRVPMMH |
| | | domain- | marcescens | | QTESSECGLACLAMICGYYGKNIDL |
| | | containing | | | IALRRQFNLSSRGATLAGLTGIAEQ |
| | | ABC | | | LRLSTRPLSLDLDELGALKLPCILH |
| | | transporter | | | WEFNHFVVLVSVKGHRAVLHDPARG |
| | | | | | RRAVSLTELSHSFTGVALEAWPGSE |
| | | | | | FTADSVRHRIHLRTLIGSVHGLKGA |
| | | | | | LGKIFCLSLVFETINLVMPIGTQLV |
| | | | | | MDHAIPAGDRGLLSLICAGLMLFIL |
| | | | | | LRAAIGMVRAWSGLVMATLINVQWQ |
| | | | | | SGLFTHLLRLPLGYFERRKLGDIQS |
| | | | | | RFGSLDTLRSTFTTSIVGAIMDGIM |
| | | | | | VIGVLVMMALYGGWLTWVVLTFTAL |
| | | | | | YVLMRLLTYGYYRQLSEEALVREAR |
| | | | | | AGSYFMETLYGIATVKMQGMAERRI |
| | | | | | AHWLNLEIDTINTGIRVTRMDMLFG |
| | | | | | GINTFIAACDQIVILWLGTSLVIDN |
| | | | | | QMTLGMFVAFGAFRGQFSDRIGSLT |
| | | | | | EFLLQLRMMSLHNERIADIALHPRE |
| | | | | | ARKPDHPYTAGLQPLGLTTHALSYR |
| | | | | | YDSQSSAIFDGLDISVAPGESVAIV |
| | | | | | GPSGAGKTTLMKVLCGLFTPDSGRV |
| | | | | | EVNGVDIRQLGINNYHKMIACVMQD |
| | | | | | DKLFSGSIRENICGFADEVDDAWMQ |
| | | | | | ACARASYLHDVILRMPMGYETLIGE |
| | | | | | LGEGLSGGQKQRLFIARAIYKKPAI |
| | | | | | LFLDEATSALDKESEAVVNQAIKRL |
| | | | | | NITKLIIAHRETTIASADRVIHLG |
| 1 | WP_099032728.1 | peptidase | Serratia | 59 | MCRGHFVNKVYENIVGKLNLSLRRK |
| | | domain- | marcescens | | VPQVLQTEAAECGLASLAMVCGYYG |
| | | containing | | | MHIDMLSMRQKFDISARGATLSSLI |
| | | ABC | | | AIAENLNLKTRALSLSLDEIHQVKR |
| | | transporter | | | PCILHWDMNHFVVLVNIRGGRITLH |
| | | | | | DPAFGRRVIGLQEFSLHFTGIALEV |
| | | | | | WPSSDFTPVVQKNRLHFRKLLSNVT |
| | | | | | GLKSALVKIFALSLVIEAVNLLMPV |
| | | | | | GTQLVMDHVIQAGDHNLLVIICVGL |
| | | | | | LFFILFRTCVSMFRSWISIVMGALI |
| | | | | | DIQWKSGLFDHLMKLPLAYFEKRKL |
| | | | | | GDIQSRFGSLDAIRTTFTTSIVSSI |
| | | | | | IDGIMSVGVFIMMFMYGGWLVWVAS |
| | | | | | GFTVLYVLLRLSTYRYYRQASEEQL |
| | | | | | VKSAKASSHFMETLYSIATLKSLGL |
| | | | | | AGTRSQFWLNLNIDTANANIRVTKL |
| | | | | | DMFFGGVNAFLAACDQIVILWLGAS |
| | | | | | LVIDSQMTLGMFVAFNAYRGQFSDR |
| | | | | | ASNLIDMVIRLRMLNLHNERLADIV |
| | | | | | LSTTEEEKPYRKICDPNEAVTFEVR |
| | | | | | DLLYQYDSLSRPVIPGLSLQIAAGE |
| | | | | | SVAIVGPSGVGKTTLMKLMCGLLEP |
| | | | | | TEGRLFINGINIHDIGINNYRECIA |
| | | | | | CVLQEDKLLAGSIAENICSFDAHPD |
| | | | | | TEFIVECAKHCNIHDDIMKMPMGYE |
| | | | | | TLVGELGGSLSGGQKQRVLIARALY |
| | | | | | RRPSILFMDEATSHLDLDNEKRVNE |
| | | | | | AISSLKMTRVIIAHRPSTIASADRV |
| | | | | | ITLQPVNAMA |
| 1 | WP_004946921.1 | peptidase | Serratia | 60 | MSKFSVKKLLSQLDMRFRHRVPLVH |
| | | domain- | plymuthica | | QTESSECGLACLAMICGHYGKNVDL |
| | | containing | | | IALRQQFHLSARGTTLSGLTDIAEQ |
| | | ABC | | | LGLSSRPLSLDIDELSALKMPCILH |
| | | transporter | | | WEFNHFVVLVSIRHNCVVLHDPALG |
| | | | | | RRIISLVEVSHSFTGVALEVWPSSA |
| | | | | | FEAHAVHKRLSLATLIGSVHGLRGA |
| | | | | | LGKIFCLSLVIETINLAMPVGTQLV |
| | | | | | MDHAIPAGDRGLLTLICVGLLLFIL |
| | | | | | LRAAVSMVRAWSSLVMTTLINVQWQ |
| | | | | | TGLFNHLLRLPLGYFERRKLGDIQS |
| | | | | | RFGSLNALQSTFTASIIGAIMDSIM |
| | | | | | VIGVLVMMVLYGGWLTWVVIAFTTV |
| | | | | | YVLLRLLTYGYYRQLSEEALVRNAR |
| | | | | | ASSYFMETLYGIATVKMQGMAERRS |
| | | | | | THWLNLEIDTINTGIRVTKMDMLFG |
| | | | | | GINTLVAACDQVVILWLGTSLVIDN |
| | | | | | HMTIGMFVAFGAFRGQFSDRIGSLT |
| | | | | | NFLLQLRMISLHNERIADIALHPRE |
| | | | | | NRKPDIHYEARFQPVSLATCALSYR |
| | | | | | YDSQSPAIFSGLDITIAPGESVAIV |
| | | | | | GPSGSGKTTLMKVLCGLFPPDSGRV |
| | | | | | EINSIDIQQLGINNYHKMIACVMQD |
| | | | | | DKLFSGSIRENICGFADNVDDTWMQ |
| | | | | | ECARASYLHEVIIRMPMGYETLIGE |
| | | | | | LGEGLSGGQKQRLFIARAIYKKPAI |
| | | | | | LFLDEATSALDKESEEVVNEAIKGL |
| | | | | | DITRIIIAHRETTIASADRVIYLD |
| 1 | WP_063919748.1 | peptidase | Serratia | 61 | MNKPTFKQRVSQLDLRLRHRVPMVH |
| | | domain- | surfactantfaciens | | QTESSECGLACLAMICGYYGKNIDL |
| | | containing | | | IALRRQFNLSTRGATLVGLIGIAEQ |
| | | ABC | | | LGLSTRPLSLDLDELGALKLPCILH |
| | | transporter | | | WEFNHFVVLVSVKGHRAVLHDPARG |
| | | | | | RRAVSLAELSHSFTGVALEAWPGSA |
| | | | | | FTADSVRHRIHLRTLIGSVHGLKGA |
| | | | | | LGKIFCLSLVFETINLVVPIGTQLV |
| | | | | | MDHAIPAGDRGLLTLICVGLMLFIL |
| | | | | | LRAAIGMVRAWAGLVMSTLINVQWQ |
| | | | | | SGLFTHLLRLPLGYFERRKLGDIQS |
| | | | | | RFGSLDTLRSTFTSSIVGAIMDGIM |
| | | | | | VIGVLVMMVLYGGWLTWVVIAFTAL |
| | | | | | YVLMRLLTYGYYRQLSEEALVRNAR |
| | | | | | AGSYFMETLYGIATVKMQGMAECRI |
| | | | | | AHWLNLEIDTINTGIRITKMDMLFG |
| | | | | | GINTFVAACDQVVILWLGTRLVIDN |
| | | | | | QMTLGMFVAFGAFRGQFSDRIGSLT |
| | | | | | EFLLQLRMMSLHNERIADIALHPRE |
| | | | | | DRKPDLPYISKMQPMGLSTHALSYR |
| | | | | | YDSQSPAVFDALNISVAPGESVAIV |
| | | | | | GPSGAGKTTLMKVLCGLFPPDSGRV |
| | | | | | EVNGVDIRQLGINNYHKMIACVMQD |
| | | | | | DKLFSGSIRENICGFADEMDDAWME |
| | | | | | VCARASYLHEVIMRMPMGYETLIGE |
| | | | | | LGEGLSGGQKQRLFIARAIYKKPAI |
| | | | | | LFLDEATSALDKDSEAKVNQAIKKL |
| | | | | | KITRIIIAHRETTIASADRVIRLD |
| 1 1 | WP_032897386.1 | MULTISPECIES: | Yersinia | 62 | MIASTMKGYFEALRQRLPVVMQTEA |
| | | peptidase | | | TECGLACMAMVAGYYGLNIDLQALR |
| | | domain- | | | KHYQVSLKGMSLRDLIVLADRLSLG |
| | | containing | | | SRPVRADLDAVPQLKTPCILHWSFN |
| | | ABC | | | HFVVLKKFSRHGAIIHDPAKGERRI |
| | | transporter | | | SITELSQHFTGIALEIWPNQRFQRR |
| | | | | | TEKQVIRLLDMFKNVSGLSRALAHV |
| | | | | | LALSFAIELLTMAVPMAIQFTVDMA |
| | | | | | LRSGDINLVTLIVWGIVALLIFRAL |
| | | | | | LSLVRSWTLMSVKYSLGLQWSSGLF |
| | | | | | SHLLRLPASYFEKRHIGDVSSRFNS |
| | | | | | LSVVQDAFTAEMVASLLDIVVVIGL |
| | | | | | LFLMSVYNGYLTAAVVSLSCIYATI |
| | | | | | KFCLFRAYRSANLEAIAHEAKQHSH |
| | | | | | FLETVRGINGVKIFNLAERRRSDWL |
| | | | | | NLVVDEANAKIYLFKIDLATQTVGL |
| | | | | | LLIGISSAMVLWLGAKLIDVGTMTT |
| | | | | | GMLFAFLIYSDMYITRTISVVDSII |
| | | | | | KLRLIDMHSERLSEVALTPPESDEG |
| | | | | | EMNLVMPTPLAGTIDVKELSYRYGD |
| | | | | | GEPAVFENISLSIKAGESIAIVGPS |
| | | | | | GCGKSTLLKTMGGLAPQESGTILLD |
| | | | | | GIDVRRLGLEAYRKHIAYVLQEDRL |
| | | | | | FAGSLLDNISSFDIHPDSEWVFECA |
| | | | | | RLASIHSEIEAMPMKYETMVGDMGS |
| | | | | | ALSGGQRQRISLARALYKRPRILFL |
| | | | | | DEATSDLDIDNEARINDSIRALKIT |
| | | | | | RIFVAHRPSMIAMADRVFDLSTNTE |
| | | | | | KEKETPHALFTERKHHVIE |
| 1 | WP_002223915.1 | peptidase | Yersinia | 63 | MNDTHFNEIKNRLNFSFRRKVPQVL |
| | | domain- | pestis | | QTEAAECGLACLVMTCRYHGMDIDL |
| | | containing | | | FNLRQRFGISSHGATLALLINISAQ |
| | | ABC | | | LKFKTRALSLDLDELRQLKTPCILH |
| | | transporter | | | WDMSHFVVLVAVKGTRFIIHDPAFG |
| | | | | | RRTVSLSEMSQHFTGVALELWPNSE |
| | | | | | FTRQKSRTRLSLLSLMRNISGLPGF |
| | | | | | LTKIFCLSLMVEAINLLLPVGTQLV |
| | | | | | MDHVIIAEDYDLLALICIGLLFFIL |
| | | | | | FRTFLSMLRSWTSLVMGSLVDVQWK |
| | | | | | AGLFDHLLKLPLAYFEKRKLGDIQS |
| | | | | | RFGSLDIIRSTLTNNVVNGIIDGLM |
| | | | | | SIGVFIMMFLYGGWLVWVVLGFTAM |
| | | | | | YMILRLATYNQYRQASEEQIVKNAK |
| | | | | | ASSHFMETLYGISTLKALGLAATRS |
| | | | | | QFWLNLNIDTTNANIRLTKLDMFFG |
| | | | | | GVNTLIGTIDQVVILWLGASMVIDG |
| | | | | | QMTLGMFVAFNAYRGQFSDRATNLI |
| | | | | | NMVLQLRMLALHSERIADIVFTETE |
| | | | | | KEQTPRQLLSPNQPAVFEARNIAFQ |
| | | | | | YDNLSKPIFSDLNIHVEAGESVAIT |
| | | | | | GPSGIGKTTLMKVIAGLLTPSQGHI |
| | | | | | LIDGLDITTVGLNNYRDCIACVLQD |
| | | | | | DKLFAGSIADNIASFDVNKDEQRIL |
| | | | | | SCANHCNIHKEIMHMPMGYETLISE |
| | | | | | LGGSLSGGQKQRLLIARALYRQPSL |
| | | | | | LFLDEATSHLDLANEAHINNAIASL |
| | | | | | KITRIFIAHRPSTIASAQRIINLEK |
| | | | | | QNVS |
| 2 | ER18_PCAT_cvaB | peptidase | Mixta | 64 | MKFLEALNFGWRRQLPVMQQTQATE |
| | | domain- | gaviniae | | CGLTCVGMIANYFGHAIDMVTLRKR |
| | | containing | | | FPTSLKGATLADVMLIAHQLGMAGR |
| | | ABC | | | ALRLELDELGKLRRPCILHWEMNHF |
| | | transporter | | | VVLKSVAKGKITIHDPARGKRDVPM |
| | | | | | DEVSRSFTGVALELLPAATFTQAQE |
| | | | | | KQSISMRKLIGNVTGIRSAFAQVMI |
| | | | | | LSVALELFGILSPFYMQWVMDQVLV |
| | | | | | SANYDLLTLLGCGFIIVTLLNIAIS |
| | | | | | ALRSWVTTWFSSLLSVQWTANVCAH |
| | | | | | LLGLPMAYFESRHVGDVLSRFGSIG |
| | | | | | TIQNTLTGRFISSILDGVMAVVTLG |
| | | | | | MLFIYNIRLAWLVLGLLAAYVIIRW |
| | | | | | ISFRPFRQANEDQIAASARAQSQLL |
| | | | | | ESIRGVQAVKLNNKQEMRVAAYANA |
| | | | | | LVESTNKGVAIQRLSIGFSSVQGAI |
| | | | | | SGVGRIVLIWMAALQVLEGNFTSGM |
| | | | | | LVAFISFSDQFISRASGLVDAVIDF |
| | | | | | TMLRLHGERLADIVLTDREADMETL |
| | | | | | LPAPMHAAQSAPSLTIKDLSFRYAE |
| | | | | | TEPYVLRHCDIAVAAGESVAIIGPS |
| | | | | | GQGKTTLAKLMMGLLKPESGCIELD |
| | | | | | GVDIKKLGMRHYRARLGCVMQDDIL |
| | | | | | FAGSLSDNICFFDAQPDQEKIEDAA |
| | | | | | KVAQIHDDISAMPMGYHSLVGDMGS |
| | | | | | SLSGGQIQRVLLARALYRQPDLLIL |
| | | | | | DEASSHLDVDRERMINQAIRHMPVT |
| | | | | | RIIIAHRPETICSADRIILLNQGKA |
| | | | | | VEIDKAHYARLSNTSPQSR |
| 2 | WP_001269710.1 | peptidase | Acinetobacter | 65 | MRYLDRLSLGLGRKVPVILQTESSE |
| | | domain- | baumannii | | CGLACLAMIAGYYGYESDLLTLRQK |
| | | containing | | | YPISQKGATLTTLVKIANKLHLTTR |
| | | ABC | | | PLKLELDELNQLRLPCILHWDLNHF |
| | | transporter | | | VVLKSVSATKITIVDPAFGEKVFTY |
| | | | | | DQASSHFTGIALELWPDSNFEEKKD |
| | | | | | KTSIRIFKLFGEIRGLWKSLGQILI |
| | | | | | LALVLEVFSLVSPFFMQWVIDHAIV |
| | | | | | SADLNLLTTLAIGFGLLMILSSLIS |
| | | | | | LLQAWVVMHMATTLNVQWKANIFHH |
| | | | | | LVNLPTNFFQKRHLGDIISRFGSID |
| | | | | | AIQRTLTTSFITAILDGLMTIFTLI |
| | | | | | LMFLYSTKLAWIAVITMLLYAVIRW |
| | | | | | IWYYPLRRATEDQIVHAAKQNTHFM |
| | | | | | ETMRGIKTIKQFEKQDYRQATWLSL |
| | | | | | FVDQINAGLTTQKLNLMFGFVNTLL |
| | | | | | FGLQNIIIVWLGATLVIEGEFTVGI |
| | | | | | LMAFLAYKNQFGGRVSSLIDKYVEV |
| | | | | | KMLSLHGERLADIVLTDQEKTDSYS |
| | | | | | FVPELSEQQKTGIDVKSLRFRYSED |
| | | | | | ESWIIHGINFNIPEGQSVAIVGPTG |
| | | | | | CGKTTLMNLLLGNLTPEHGEIKIGG |
| | | | | | QSIKNIGSNKLRNFIAYVAQDDVLF |
| | | | | | AGSILENISFFDDGAQQDWVEQCAK |
| | | | | | MASVHDEIMAMPMGYQTLVGDMGNI |
| | | | | | LSGGQKQRILLARALYRKPKILFLD |
| | | | | | EATSHLDIIKEKEINNMIKSLNITR |
| | | | | | IIIAHRPETISSVDRIIALNNGQIV |
| | | | | | SDQML |
| 2 | WP_075703216.1 | peptidase | Acinetobacter | 66 | MRYLERLSLGLGRTVPVILQTESSE |
| | | domain- | baumannii | | CGLACLAMIAGYYGYESDLLTLRQK |
| | | containing | | | HPISQKGATLATLVKIANKLHLTTR |
| | | ABC | | | PLKLELDELNQLRLPCILHWDLNHF |
| | | transporter | | | VILKNVSPAKITIVDPAFGERVFTY |
| | | | | | DQASSHFTGIALELWPDSNFEEKKD |
| | | | | | KTSIRIFKLFGEIRGLWKSLGQILI |
| | | | | | LALVLEVFSLVSPFFMQWVIDHAIV |
| | | | | | SADLNLLTTLALGFGLLMLLSSLIS |
| | | | | | LLQAWVVMHMATTLNVQWKANIFHH |
| | | | | | LVNLPTNFFQKRHLGDVISRFSSID |
| | | | | | AIQRTLTTSFITAILDGLMTIFTLI |
| | | | | | LMFIYSVKLACIALISMLMYIIIRW |
| | | | | | IWYYPLRRATEDQIVHGAKQSTHFM |
| | | | | | ETMRGIKTIKQFEKQDYRQATWLSL |
| | | | | | FVNQINAGLTTQKLNIMFGFVNTLL |
| | | | | | FGLQNIIIVWLGATLVIEGEFTVGI |
| | | | | | LMAFIAYKNQFGDRVTSLIDKYVEV |
| | | | | | RMLSLHGERLADIVLTDQEKTDNYL |
| | | | | | FVPESSELQKTEIDVKSLRFRYSEE |
| | | | | | ESWIIHGINFNIPEGQSVAIVGSTG |
| | | | | | CGKTTLMNLLLGNLNPEHGEIKIGG |
| | | | | | QSIKSLGSNRLRNFIAYVAQDDVLF |
| | | | | | AGFILENISFFDDSVQQDWVEQCAK |
| | | | | | MASVHDEIMAMPMGYQTLVGDMGNI |
| | | | | | LSGGQKQRILLARALYRKPKILFLD |
| | | | | | EATSHLDIIKEKEINDMIKSLNITR |
| | | | | | IIIAHRPETISSVDRIIALNNGRIV |
| | | | | | SDQMYEPL |
| 2 | WP_104949566.1 | peptidase | Enterobacter | 67 | MDFIEKIRFSARKSLPLLRQTQAAE |
| | | domain- | sp. | | CGLACIGMIAGYYGYKMDMLTLRKK |
| | | containing | SGAir0187 | | YAMSLKGSTLTDIINLAGHLGLVTR |
| | | ABC | | | ALRLEVEELSQLRLPAILHWDMKHY |
| | | transporter | | | IVLSEIHGDKVTIHDPARGRRILKI |
| | | | | | KDLAASFTGIALELVPQSDFEKREE |
| | | | | | KESLSMLKLLGNISGIKSVFAQLMI |
| | | | | | LTLSLEIFGIITPFYMQWVLDQVLV |
| | | | | | SSDYNLMTLLGCAFIIVVLLQNGIT |
| | | | | | ALRTWVTTWFSSLLSVQWSSNICSH |
| | | | | | LLNLPQSWFEERHMGDIVSRFGSIN |
| | | | | | NIQSTLTTRFISSVFDGVMSIITLG |
| | | | | | IIVVYSPSLSLIVVGLFLAYTTVRM |
| | | | | | VAFQPFRQASEDQMVANSQVQSQLI |
| | | | | | ESIRGSQAIMLSNKQNMRVSTYTNE |
| | | | | | LVEATNNGIKIQRLSIFFTTVQGIL |
| | | | | | SGVGRIVLIWLAALQVLDGHFSSGM |
| | | | | | LMSFITFSDQFISRASGLIDAFIEF |
| | | | | | RMLRLHGERLSDIVLSERETDMLNL |
| | | | | | SAHPDPIHAVSVEFKNLSFRYSPTE |
| | | | | | PWILKQVNMLIHAGESVAIVGPSGQ |
| | | | | | GKTTLARLLLGLLRAEEGAIIIDGV |
| | | | | | ENVRLGMTWVRNNTGSVMQDDQLFA |
| | | | | | GSILDNICFFDADFDMSMVEEAAKI |
| | | | | | AQVHTDIIAMPMGYNSLVGDMGSAL |
| | | | | | SGGQIQRLLLARALYKKPSILLLDE |
| | | | | | ATSHLDIYREKAINEAIRKMSITRI |
| | | | | | IIAHRPETIMSADRIFVVANHKINE |
| | | | | | VDKASLFGIQNGEMKPALT |
| 2 | WP_113650898.1 | peptidase | Escherichia | 68 | MYMSKLSFSIKKNFPFFYQNADKDC |
| | | domain- | albertii | | ALACIKMLASYYKKEHLLNCLSQQA |
| | | containing | | | HNKKTFWSVNDILSALRTLGMNPRP |
| | | ABC | | | VRLELDEINSLSIPSVLHWNMNHFV |
| | | transporter | | | LLVGKKKNKLIIHDPEKGIRHLSLK |
| | | | | | ELSSSFTGVAVEVYNDKIISEKKTS |
| | | | | | IKDNKVMHYFFMAISLGRKNFIYLF |
| | | | | | ILILLSEIISITLPQITQLVLDNVI |
| | | | | | INSDYQLLLTAVTFYLFLHVINTGI |
| | | | | | LATRDWLIIWLNANINAQWGINFYN |
| | | | | | RLVLLKRNYFSSRSTGDILSRFSSL |
| | | | | | EYIRNVIISKTTTSLLDFFMAIGSL |
| | | | | | IIMLTYSLNLTFIVIFSTGVYFLLK |
| | | | | | ILYFGSVKHFNLGTIRIKAQQQSSL |
| | | | | | IEAVKYNQTIKLYFDEYMSAGKYVG |
| | | | | | ELIEGVNVKTQIDILNIIFSASNRI |
| | | | | | LTALKNICILYFGGVFVIKTSFTIG |
| | | | | | MLVAFITYSEQFSRRVTSLIDFFVQ |
| | | | | | IGITRLHVSRVSDISDADYETISAD |
| | | | | | MPKRISSPLQVEFNNVSVKHPDKQI |
| | | | | | NILNRINFKIMPGETVILKGISGCG |
| | | | | | KTTLLNAILGLIDVSSGHISITENG |
| | | | | | MKKSCIDARKMTGAVLQGDSLLNGT |
| | | | | | VIYNITFNDSSPSRDVIDLTKMIGV |
| | | | | | HDVIKSLPMGYHTQISDSDHILSAG |
| | | | | | QKQKILLARAMYRKPQLLILDEATS |
| | | | | | NLDIDSEYQISQAILSLNCTKIIVS |
| | | | | | HKEKSFFMADKVITIKNGMIVNIHE |
| | | | | | VIKNQHL |
| 2 | WP_013331803.1 | peptidase | Halomonas | 69 | MNLSELPKRLLGGIRRGTPVIIQSE |
| | | domain- | elongata | | AAECGLACMAMIASHHGHALDLPAM |
| | | containing | | | RHRFSTSLKGMALRDLLKVGGDLKL |
| | | ABC | | | STRAVRLELSDLGKLQLPCVLHWNH |
| | | transporter | | | NHFVVLTRVGTNSVTIHDPATGSRK |
| | | | | | LPFSEVSKRFTGVALEAWPNELFTK |
| | | | | | KNERERIHVTEMIKRTSGIGRAALQ |
| | | | | | IIAISVLLEIVTIAMPIGFQMVIDQ |
| | | | | | VIVAQDYDLLTLITIAFVLLLILQV |
| | | | | | LASFVRSWSSMLIGSSLVLQWKAGL |
| | | | | | FDHLMRLSLSYFEKRHVGDIVSRFG |
| | | | | | SLDSIQKTLTTSAIMALLDGVMSIA |
| | | | | | LIVMLWLYGGWLASVPLIAVLLYTI |
| | | | | | IRLCLYHPYRNLSEEAIVYAAQENT |
| | | | | | HEMESLRGMASLKVLNLEERRRSVW |
| | | | | | INYLVDRVNANLQVEKFDIVFQNAS |
| | | | | | RFLFGVDRIIIIYFGARAIMGDALS |
| | | | | | VGMLIAFLSYKDQFTNRMDNFVSTM |
| | | | | | IKLRMLSLHGERVADIALIEPEKSD |
| | | | | | LSSGSTE |
| 2 | WP_108899385.1 | peptidase | Limnobaculum | 70 | SEREEEGAGSIDVKNVSFRYADSEE |
| | | domain- | parvum | | NVLKDVSFSIDAQECVGISGPSGSG |
| | | containing | | | KTTLLKILSGLMLPSEGHVCIDGKE |
| | | ABC | | | LSSIGAAAYRDRVGCVLQDDRLFAG |
| | | transporter | | | SVAENISGFDPDPDTRRIHQCAMLA |
| | | | | | AIHDEIAKMPMGYETFVGDMGSTLS |
| | | | | | GGQMQRIILARALYRRPKVLLLDEA |
| | | | | | TSHLDPENEAAINAAIQKLSITRVV |
| | | | | | IAHRPSTLAMTDRVIDMQALQGGAM |
| | | | | | SFVKKINFGLTSQRLPVLIQTESAE |
| | | | | | CGLACLAMIAGYHNYNIDLATLRGK |
| | | | | | YSISQKGANLSQLLNISKQMNLSSR |
| | | | | | SLRLEMEDLPKLKLPCILHWEFSHF |
| | | | | | VVLKAIKGNKCIIHDPAVGMRQLAF |
| | | | | | TEVSKSFTGVAVELWPDDGFKPQEV |
| | | | | | RRQVRFRDLIGKITGLKSSFLQVLL |
| | | | | | LALGIELFSIISPLFTQWTIDYVIV |
| | | | | | SNDRNLLVTLIVGFGVMMLVQQLTS |
| | | | | | AVRAWVMMYISTSISVQWRDSVFHH |
| | | | | | LLRLPISYFEKRHLGDIVSRFGAVD |
| | | | | | AIQSTLSSSFFVAILDGLMTIVTLV |
| | | | | | MMYIYSPYLATICLITMVLYIIGRI |
| | | | | | FWYRPLRMATEEQIIHGAKQQSHFL |
| | | | | | ETIRGVRTIKLFQRQNERMSDWMAL |
| | | | | | LISQINSGVRVQKLQIYYQQLNGLL |
| | | | | | FGIENLVVLGVGATMVMDQTFTVGV |
| | | | | | LMAFSSYKGQFSSRISSLVDKFFEL |
| | | | | | KMLSIQTERLADIVLTEPEDDIEQK |
| | | | | | QYVQQTQPPRIEFDNVSFRYSEHEP |
| | | | | | YVIKDTSFVIESGECVALAGASGCG |
| | | | | | KTTLSQLLCGSLVPVSGDILIDGVS |
| | | | | | LKQLTVANLRRFSATVLQDDVLFAG |
| | | | | | SIMDNITFFDHNINHEKAIKCAMMA |
| | | | | | AIHHDIEQMPMQYNTLVGDMGVALS |
| | | | | | GGQKQRILLARALYKQPQLLILDEA |
| | | | | | TSHLDVQLEQLVNTAVSQLAITRLV |
| | | | | | IAHRPQTLAMVDRIIVIENGTVGHD |
| | | | | | LTPAQLFSSAAPSQGSAAEQGS |
| 2 | WP_052142241.1 | MULTISPECIES: | Methylomonas | 71 | MQHILDFSGRRKTPLILQTEAAECG |
| | | peptidase | | | LACLAMVANFHGHRVDLASLRQKFS |
| | | domain- | | | LSLKGSTLNHLIQIANQLHLGSRPV |
| | | containing | | | RVDLNELSKLSLPAILHWDFNHFVV |
| | | ABC | | | LTQVKRGVATIHDPAKGTVNLPFTE |
| | | transporter | | | VSKHFTGIALELQPTQAFTAKTERQ |
| | | | | | QIKLSRLIGQLNGASSATLQIILLA |
| | | | | | AAIEVFAIVAPFFMQLVVDNAVVAR |
| | | | | | DTNLLTVLGLGFLLLALIQIGITAL |
| | | | | | RSWVVLVLSTSVNLQMMSNLFGHLL |
| | | | | | QLPMNFFEKRHLGDIVSRFESLNVI |
| | | | | | QRTLTTSFLEAIIDGVMALVTLGMM |
| | | | | | LVYSWKLAAIVSLAALLYGLLRLVS |
| | | | | | FAPLRAASEEQILRAAKQQTNLLET |
| | | | | | VRGMQSVKLFNRQLQRRHGYQNLMV |
| | | | | | DHFNAGIRVQKLNIIYRALNSLLFG |
| | | | | | VENIAVIWLGALFILDGGFSVGMLF |
| | | | | | AFMSFKDQFTHRVGSFIEKGIEFKM |
| | | | | | LGLHTERVADIALAEPEQEHCSGAR |
| | | | | | QDQLPASIQIRNLDFSYAPSEPPVL |
| | | | | | KNLNLDFAEGESVAIIGPSGYGKTT |
| | | | | | LAKLILGLLQPSAGEILIGGVRLNR |
| | | | | | IDSHDYRNMVAAVMQEDQLFAGSIA |
| | | | | | DNICFFDPEPDQAWIETCAQLAAVH |
| | | | | | QDVMTMPMAYNTLIGDMGTVLSGGQ |
| | | | | | KQRVLLARALYKRPKILVLDEATSH |
| | | | | | LDVAREKLVNGAVQQLKLTRIIIAH |
| | | | | | RPETIASVDRVIDLQAIADAALGEQ |
| | | | | | HYVSQAA |
| 2 | WP_071888632.1 | peptidase | Pantoea | 72 | MLLLEKLNFKWFNRLPMIRQSQAAE |
| | | domain- | vagans | | CGLACLGMVANYHGHQIDMITLRRQ |
| | | containing | | | FATSLKGATLADVIAIAQQLNMTSR |
| | | ABC | | | ALRVELEELSKLRMPCILHWELNHF |
| | | transporter | | | VVLKKVRGNKITIHDPARGIRQLTF |
| | | | | | KEASTAFTGVALELVPSSTFEVKEE |
| | | | | | KESISMMKLVGSVTGVKSAFAQVLI |
| | | | | | LSIALELFGVLGPFFMQWVMDMVLV |
| | | | | | SADYSLLSLLGVGFIMIALFQTIVT |
| | | | | | ALRSWVMNWFSSQLSVQWTINVCHH |
| | | | | | MLKLPLEWFESRHVGDILSRYGSLN |
| | | | | | TIQSTLTSRFISTVLDGVMSIVTVV |
| | | | | | MLFIYNAQLAWLVIGLFLAYALLRF |
| | | | | | MAYDPVRRANEEQIISSARTQSSLL |
| | | | | | ETLRGIQAVKTNNKQVPRLSAYMNF |
| | | | | | LVDTTNKGIVIQRLNILFGSAQGLL |
| | | | | | TSVGRVVLVWLAALQVLDGNFSAGM |
| | | | | | LTAFISFSDQFMSRGSGLINAIIDF |
| | | | | | RMLRMHGERLADIVLSETEASSEGN |
| | | | | | PGLVSKETDKETDVPQDIKLINIRF |
| | | | | | RYAPTEPWIVDGANLEIKAGESLAI |
| | | | | | VGPSGQGKTTMAKIILGLLHPEEGT |
| | | | | | ITVGGMDITQTGLEHHRNRIGCVMQ |
| | | | | | DDILFSGSISENISFFDNEPDHAKI |
| | | | | | TRVARLAQIHGDIMKMPMNYQSLVG |
| | | | | | DMGSFLSGGQLQRILLARALYREPK |
| | | | | | ILVLDEATSHLDIYNEAQINNAIKQ |
| | | | | | MKITRIIIAHRPETIRSADKIVLLN |
| | | | | | NGTLSEVTAEQLFGQAATHNETEIT |
| | | | | | YG |
| 2 | WP_125561120.1 | peptidase | Pseudoalteromonas | 73 | MHVEDESPVQQLQFWSRKSLPVILQ |
| | | domain- | rubra | | SEAAECGLASLAMVAAYHGYHSDLT |
| | | containing | | | SLRQKFSISIEGATLLDIMHFAEKL |
| | | ABC | | | EMSSRPLRIELEDLDALQTPCILHW |
| | | transporter | | | DMNHFVVLKKANEKRIVIHDPASGE |
| | | | | | KTYTMAEASKHFTGVALELTPTKSF |
| | | | | | EKKEKKPSLQFSDFWSRITGLKRSL |
| | | | | | TLIFLLSILLQIFTLAAPYYIQLVI |
| | | | | | DDVVLTGDTNLLTVLATGFFLVLVF |
| | | | | | EIATNALRGFTLLHFGNQMNIQLGA |
| | | | | | NLFHHLVRLPISYFEKRHMGDVVSR |
| | | | | | FGSLQQVKQILTTGVIEAIIDGLMA |
| | | | | | IITLAMIFFYSPTLSAVVLAAVVAY |
| | | | | | AIVRIAMYRPFRNISEQEIMARAEE |
| | | | | | NSNFMETVRGIQTIKLFGSEVKREG |
| | | | | | QWQNRYANAINQSIRLGNFQIGYDA |
| | | | | | INRALFGIENILVVYLAAHLVLDGG |
| | | | | | FSTGMLFAFMSYKRQFMDKTANLIE |
| | | | | | KLIEFKMVGLHFDRIADIALTDKEI |
| | | | | | LQPDQVKQHQVKGQIELRNISFSYS |
| | | | | | DATPNVLNDLNLTIEAGESVAITGP |
| | | | | | SGCGKSTLLKIMLGLNLPKSGEILI |
| | | | | | DGVPMEQIGARQYRQQIAAVMQDDE |
| | | | | | LLSGSVADNIAFFDTPIDMERVVYC |
| | | | | | AQLAAIHDDISQMPMNYDSLIGDMG |
| | | | | | SSLSGGQKQRIILARALYKQPKILF |
| | | | | | MDEATSHLDTNLESDINEAVSRLDI |
| | | | | | TRVIIAHRKETIASADREVRLKKPD |
| | | | | | IPDPYAQEEESVCE |
| 2 | WP_003093498.1 | MULTISPECIES: | Pseudomonas | 74 | MAFLDALALRLGRRLPLVLQTEATE |
| | | peptidase | | | CGLACLAMIAGYHGHHTGLMELRRR |
| | | domain- | | | FSVSLKGISLKQLIQTAHRLGLGTR |
| | | containing | | | AVKLDLGDLGKLKLPCVLHWNFNHF |
| | | ABC | | | VVLKAVDGRGAVLHDPAHGQRRLGL |
| | | transporter | | | EEVSRSFTGVALELWPESGFEKQEA |
| | | | | | PPRIKLLGMLGKVTGLYRSLAQVLL |
| | | | | | LAGALEVFSLISPFFLQWTIDNVIV |
| | | | | | SEDRDLLSTLAIGFGLLLLMQQAVS |
| | | | | | GVRAWVMMHMSTLLGVQWQANVFSH |
| | | | | | LLRLPAQYFEKRHLGDVVSRFGAVN |
| | | | | | SIQQTLTAAFLSAVLDGLMTVATLG |
| | | | | | MMLLYSPPLAAIAIAAMSLYALGRW |
| | | | | | IWYRPLRNATEEQIVHAARQQSHFL |
| | | | | | ETVRGIRPLKLFQRQDERRSVWLGL |
| | | | | | LVEQINAGLRTQKLQLFYQQLNGLL |
| | | | | | FGVENLLVIWLGATMVMDGQFSVGI |
| | | | | | LMAFNAYKSQFDSRVGSLIDKFFEL |
| | | | | | RMLQLQGERLADIVLQAPEVSHGDI |
| | | | | | LPENLREREASIEIQGLRYRYAEQE |
| | | | | | PWVLDGLDLRIAGGESVAIVGPSGC |
| | | | | | GKSTLLNVLLGILPPVEGQIRMAGL |
| | | | | | DLAQLGLDGLRELVGTVLQDDVLFA |
| | | | | | GSLSDNISFFDPQPDMPWLLQCAQM |
| | | | | | AAIHDDIQAMPMGYNTLVGDMGTVL |
| | | | | | SGGQKQRVMLARALYKKPRILFLDE |
| | | | | | ATSHLDVHCEQRVNAAIRALRITRI |
| | | | | | MVAHRPETIASADRVIVLGQGKVSL |
| | | | | | DESTARLAERQAAAAREQA |
| 2 | WP_003105688.1 | MULTISPECIES: | Pseudomonas | 75 | MAFLDALALRLGRRLPLVLQTEATE |
| | | peptidase | | | CGLACLAMIAGYHGHHTGLMELRRR |
| | | domain- | | | FSVSLKGISLKQLIQTAHRLGLGTR |
| | | containing | | | AVKLDLGDLGKLKLPCVLHWNFNHF |
| | | ABC | | | VVLKAVDGRGAVLHDPAHGQRRLGL |
| | | transporter | | | EEVSRSFTGVALELWPESGFEKQEA |
| | | | | | PPRIKLLGMLGKVTGLYRSLAQVLL |
| | | | | | LAGALEVFSLISPFFLQWTIDNVIV |
| | | | | | SEDRDLLSTLAIGFGLLLLMQQAVS |
| | | | | | GVRAWVMMHMSTLLGVQWQANVFSH |
| | | | | | LLRLPAQYFEKRHLGDVVSRFGAVN |
| | | | | | SIQQTLTAAFLSAVLDGLMTVATLG |
| | | | | | MMLLYSPPLAAIAIAAMSLYALGRW |
| | | | | | IWYRPLRNATEEQIVHAARQQSHFL |
| | | | | | ETVRGIRPLKLFQRQDERRSVWLGL |
| | | | | | LVEQINAGLRTQKLQLFYQQLNGLL |
| | | | | | FGVENLLVIWLGATMVMDGQFSVGI |
| | | | | | LMAFNAYKSQFDSRVGSLIDKFFEL |
| | | | | | RMLQLQGERLADIVLQAPEVSHGDI |
| | | | | | LPENLREREASIEIQGLRYRYAEQE |
| | | | | | PWVLDGLDLRIAGGESVAIVGPSGC |
| | | | | | GKSTLFNVLLGILPPVEGQIRMAGL |
| | | | | | DLAQLGLDGLRELVGTVLQDDVLFA |
| | | | | | GSLSDNISFFDPQPDMPWLLQCAQM |
| | | | | | AAIHDDIQAMPMGYNTLVGDMGTVL |
| | | | | | SGGQKQRVMLARALYKKPRILFLDE |
| | | | | | ATSHLDVHCEQRVNAAIRALRITRI |
| | | | | | MVAHRPETIASADRVIVLGQGKVSL |
| | | | | | DESTARLAERQAAAAREQA |
| 2 | WP_003137546.1 | MULTISPECIES: | Pseudomonas | 76 | MAFLDALALRLGRRLPLVLQTEATE |
| | | peptidase | | | CGLACLAMIAGYHGHHTGLMELRRR |
| | | domain- | | | FSVSLKGISLKQLIQTAHRLGLGTR |
| | | containing | | | AVKLDLGDLGKLKLPCVLHWNFNHF |
| | | ABC | | | VVLKAVDGRGAVLHDPAHGQRRLGL |
| | | transporter | | | EEVSRSFTGVALEFWPESGFEKQEA |
| | | | | | PPRIKLLGMLGKVTGLYRSLAQVLL |
| | | | | | LAGALEVFSLISPFFLQWTIDNVIV |
| | | | | | SEDRDLLSTLAIGFGLLLLMQQAVS |
| | | | | | GVRAWVMMHMSTLLGVQWQANVFSH |
| | | | | | LLRLPAQYFEKRHLGDVVSRFGAVN |
| | | | | | SIQQTLTAAFLSAVLDGLMTVATLG |
| | | | | | MMLLYSPPLAAIAIAAMSLYALGRW |
| | | | | | IWYRPLRNATEEQIVHAARQQSHFL |
| | | | | | ETVRGIRPLKLFQRQDERRSVWLGL |
| | | | | | LVEQINAGLRTQKLQLFYQQLNGLL |
| | | | | | FGVENLLVIWLGATMVMDGQFSVGI |
| | | | | | LMAFNAYKSQFDSRVGSLIDKFFEL |
| | | | | | RMLQLQGERLADIVLQAPEVSHGDI |
| | | | | | LPENLRDREASIEIQGLRYRYAEQE |
| | | | | | PWVLDGLDLRIAGGESVAIVGPSGC |
| | | | | | GKSTLLNVLLGILPPVGGQIRMAGL |
| | | | | | DLAQLGLDGLRELVGTVLQDDVLFA |
| | | | | | GSLSDNISFFDPQPDMPWLLQCAQM |
| | | | | | AAIHDDIQAMPMGYNTLVGDMGTVL |
| | | | | | SGGQKQRVMLARALYKKPRILFLDE |
| | | | | | ATSHLDVHCEQRVNAAIRALRITRI |
| | | | | | MVAHRPETIASADRVIVLGQGKVSL |
| | | | | | DESTARLAERQAAAAREQA |
| 2 | WP_015502334.1 | MULTISPECIES: | Pseudomonas | 77 | MAFLDALALRLGRRLPLVLQTEATE |
| | | peptidase | | | CGLACLAMIAGYHGHHTGLMELRRR |
| | | domain- | | | FSVSLKGISLKQLIQTAHRLGLGTR |
| | | containing | | | AVKLDLGDLGKLKLPCVLHWNFNHF |
| | | ABC | | | VVLKAVDGRGAVLHDPAHGQRRLGL |
| | | transporter | | | EEVSRSFTGVALELWPESGFEKQEA |
| | | | | | PPRIKLLGMLGKVTGLYRSLAQVLL |
| | | | | | LAGALEVFSLISPFFLQWTIDNVIV |
| | | | | | SEDRDLLSTLAIGFGLLLLMQQAVS |
| | | | | | GVRAWVMMHMSTLLGVQWQANVFSH |
| | | | | | LLRLPAQYFEKRHLGDVVSRFGAVN |
| | | | | | SIQQTLTAAFLSAVLDGLMTVATLG |
| | | | | | MMLLYSPPLAAIAIAAMSLYALGRW |
| | | | | | IWYRPLRNATEEQIVHAARQQSHFL |
| | | | | | ETVRGIRPLKLFQRQDERRSVWLGL |
| | | | | | LVEQINAGLRTQKLQLFYQQLNGLL |
| | | | | | FGVENLLVIWLGATMVMDGQFSVGI |
| | | | | | LMAFNAYKSQFDSRVGSLIDKFFEL |
| | | | | | RMLQLQGERLADIVLQAPEVSHGDI |
| | | | | | LPENLREREASIEIQGLRYRYAEQE |
| | | | | | PWVLDGLDLRIAGGESVAIVGPSGC |
| | | | | | GKSTLLNVLLGILPPVEGQIRMAGL |
| | | | | | DLAQLGLDGLRELVGTVLQDDVLFA |
| | | | | | GSLSDNISFFDPQPDMPWLLQCAQM |
| | | | | | AAIHDDIQAMPMGYNTLVGDMGTVL |
| | | | | | SGGQKQRVMLARALYKKPRILFLDE |
| | | | | | ATSHLDVHCEQRVNAAIRALRITRV |
| | | | | | MVAHRPETIASADRVIVLGQGKVSL |
| | | | | | DESTARLAERQAAAAREQA |
| 2 | WP_041924441.1 | MULTISPECIES: | Pseudomonas | 78 | MNLRNTFAFGVGRKLPVVLQAEATE |
| | | peptidase | | | CGLACMAMIASYHGQHSDLLSLRQR |
| | | domain- | | | LSPSMKGVNLKQLTHMAARLGLGSR |
| | | containing | | | ALRVELNALGQLQLPCVLHWNFNHF |
| | | ABC | | | VVLKEVNARGVVVHDPGRGLCKLTL |
| | | transporter | | | DEVSAAFTGVALELWPESDFQPAAA |
| | | | | | QPPIPLRKLLGRVQGFGRVLTQVLL |
| | | | | | LALALELCTILSPFFMQTVIDKVLV |
| | | | | | SADLDLLTVLAIGFGLLLIIQHTVA |
| | | | | | LARAWALMYLGTMLGSQWQINVFTH |
| | | | | | LLRLPVAFFERRHLGDIVSRFGSLK |
| | | | | | AIQQTVTTSFIEALLDGLMTVVTLG |
| | | | | | LMIAYSAKLALVAVVAMIIYGLARW |
| | | | | | IWFGPLRRASENQLVHAARQQSHFL |
| | | | | | ESARGVRTIKLFNRHEQRCSAWSAL |
| | | | | | LVEEINAGLHPQKLQLAYRAFNGVL |
| | | | | | FGLVTIIVIWMGARLVLEGQFSAGM |
| | | | | | LIAFNSYKEQFNGRVAGLIDKIVDV |
| | | | | | IMLRLHGERLADIVLHDAEPIAGPD |
| | | | | | TFDDACNDRVPSLEVRQLRFRYSEH |
| | | | | | EPYVLDDVSIHIRAGESVAIVGPSG |
| | | | | | GGKSTLLNVMLGILAPCAGSVLLDG |
| | | | | | LPVGPENIGRLRRITGTVLQDDVLF |
| | | | | | AGSIGDNISFFETGADQKWIEQCAH |
| | | | | | LAAVHDDIAQMPMGYNTLVGDMGTV |
| | | | | | LSGGQKQRVLLARALYKRPQLLFLD |
| | | | | | EATSHLDINREAAVNQALQTLNITR |
| | | | | | IIVAHRPETIRTADRVVALAQGRVA |
| | | | | | YDGPVLNEAPSA |
| 2 | WP_023094005.1 | peptidase | Pseudomonas | 79 | MAFLDALALRLGRRLPLVLQTEATE |
| | | domain- | aeruginosa | | CGLACLAMIAGYHGHHTGLMELRRR |
| | | containing | | | FSVSLKGISLKQLIQTAHRLGLGTR |
| | | ABC | | | AVKLDLGDLGKLKLPCVLHWNFNHF |
| | | transporter | | | VVLKAVDGRGAVLHDPAHGQRRLGL |
| | | | | | EEVSQSFTGVALELWPESGFEKQEA |
| | | | | | PPRIKLLGMLGKVTGLYRSLAQVLL |
| | | | | | LAGALEVFSLISPFFLQWTIDNVIV |
| | | | | | SEDRDLLSTLAIGFGLLLLMQQAVS |
| | | | | | GVRAWVMMHMSTLLGVQWQANVFSH |
| | | | | | LLRLPAQYFEKRHLGDVVSRFGAVN |
| | | | | | SIQQTLTAAFLSAVLDGLMTVATLG |
| | | | | | MMLLYSPPLAAIAIAAMSLYALGRW |
| | | | | | IWYRPLRNATEEQIVHAARQQSHFL |
| | | | | | ETVRGIRPLKLFQRQDERRSVWLGL |
| | | | | | LVEQINAGLRTQKLQLFYQQLNGLL |
| | | | | | FGVENLLVIWLGATMVMDGQFSVGI |
| | | | | | LMAFNAYKSQFDSRVGSLIDKFFEL |
| | | | | | RMLQLQGERLADIVLQAPEVSHGDI |
| | | | | | LPENLREREASIEIQGLRYRYAEQE |
| | | | | | PWVLDGLDLRIAGGESVAIVGPSGC |
| | | | | | GKSTLFNVLLGILPPVEGQIRMAGL |
| | | | | | DLAQLGLDGLRELVGTVLQDDVLFA |
| | | | | | GSLSDNISFFDPQPDMPWLLQCAQM |
| | | | | | AAIHDDIQAMPMGYNTLVGDMGTVL |
| | | | | | SGGQKQRVMLARALYKKPRILFLDE |
| | | | | | ATSHLDVHCEQRVNAAIRALRITRI |
| | | | | | MVAHRPETIASADRVIVLGQGKVSL |
| | | | | | DESTARLAERQAAAAREQA |
| 2 | WP_023095183.1 | peptidase | Pseudomonas | 80 | MAFLDALALRLGRRLPLVLQTEATE |
| | | domain- | aeruginosa | | CGLACLAMIAGYHGHHTGLMELRRR |
| | | containing | | | FSVSLKGISLKQLIQTAHRLGLGTR |
| | | ABC | | | AVKLDLGDLGKLKLPCVLHWNFNHF |
| | | transporter | | | VVLKAVDGRGAVLHDPAHGQRRLGL |
| | | | | | EEVSRSFTGVALELWPESGFEKQEA |
| | | | | | PPRIKLLGMLGKVTGLYRSLAQVLL |
| | | | | | LAGALEVFSLISPFFLQWTIDNVIV |
| | | | | | SEDRDLLSTLAIGFGLLLLMQQAVS |
| | | | | | GVRAWVMMHMSTLLGVQWQANVFSH |
| | | | | | LLRLPAQYFEKRHLGDVVSRFGAVN |
| | | | | | SIQQTLTAAFLSAVLDGLMTVATLG |
| | | | | | MMLLYSPPLAAIAIAAMSLYALGRW |
| | | | | | IWYRPLRNATEEQIVHAARQQSHFL |
| | | | | | ETVRGIRPLKLFQRQDERRSVWLGL |
| | | | | | LVEQINAGLRTQKLQLFYQQLNGLL |
| | | | | | FGVENLLVIWLGATMVMDGQFSVGI |
| | | | | | LMAFNAYKSQFDSRVGSLIDKFFEL |
| | | | | | RMLQLQGERLADIVLQAPEVSHGDI |
| | | | | | LPENLREREASIEIQGLRYRYAEQE |
| | | | | | PWVLDGLDLRIAGGESVAIVGPSGC |
| | | | | | GKSTLFNVLLGILPPVEGQIRMAGL |
| | | | | | DLTQLGLDGLRELVGTVLQDDVLFA |
| | | | | | GSLSDNISFFDPQPDMPWLLQCAQM |
| | | | | | AAIHDDIQAMPMGYNTLVGDMGTVL |
| | | | | | SGGQKQRVMLARALYKKPRILFLDE |
| | | | | | ATSHLDVHCEQRVNAAIRALRITRI |
| | | | | | MVAHRPETIASADRVIVLGQGKVSL |
| | | | | | DESTARLAERQAAAAREQA |
| 2 | WP_034081631.1 | peptidase | Pseudomonas | 81 | MAFLDTLALRLGRRLPLVLQTEATE |
| | | domain- | aeruginosa | | CGLACLAMIAGYHGHHTGLMELRRR |
| | | containing | | | FSVSLKGISLKQLIHTAHRLGLGSR |
| | | ABC | | | AVKLELADLGKLKLPCVLHWNFNHF |
| | | transporter | | | VVLKAVDGRGVVLHDPAHGLRRLGL |
| | | | | | DEVSRSFTGVALELWPESGFEKQEA |
| | | | | | PPRIRLLGMLGKVTGLYRSLAQVLL |
| | | | | | LAGALEVFSLVSPFFLQWTIDNVIV |
| | | | | | SEDRDLLSTLAIGFGLLLLMQQAVS |
| | | | | | GVRAWVMMHMSTLLGVQWQANVFSH |
| | | | | | LLRLPAQYFEKRHLGDVVSRFGAVN |
| | | | | | SIQQTLTAAFLSAVLDGLMTLATLA |
| | | | | | MMLLYSPPLAAIAIAAMSLYALGRW |
| | | | | | IWYRPLRNATEEQIVHAARQQSHFL |
| | | | | | ETVRGIRPLKLFQRQDERRSVWLGL |
| | | | | | LVEQINAGLRTQKLQLFYQQLNGLL |
| | | | | | FGVENLLVIWLGATMVMDGQFSVGI |
| | | | | | LMAFNAYKSQFDGRVGSLIDKFFEL |
| | | | | | RMLQLQGERLADIVLQAPEANHGDT |
| | | | | | LPDNLREREASIEIQGLRYRYSDQE |
| | | | | | PWVLDGLDLRIAGGESVAIVGPSGC |
| | | | | | GKSTLFNVLLGILPPVEGQIRMAGL |
| | | | | | ELSQLGLDGLRELVGTVLQDDVLFA |
| | | | | | GSLSDNISFFDPQPDLPWLLQCAQM |
| | | | | | AAIHDDIQAMPMGYNTLVGDMGTVL |
| | | | | | SGGQKQRVMLARALYKKPRILFLDE |
| | | | | | ATSHLDVHCEQRVNAAIRALRITRI |
| | | | | | MVAHRPETIASADRVVVLGQGKVTL |
| | | | | | DESTARLAERQAAAAREQA |
| 2 | WP_005745446.1 | peptidase | Pseudomonas | 82 | MNLRNTFAFGVGRKLPVVLQAEATE |
| | | domain- | amygdali | | CGLACMAMIASYHGQHSDLLSLRQR |
| | | containing | | | LSPSMKGVNLKQLTHMAARLGLGSR |
| | | ABC | | | ALRVELNALGQLQLPCVLHWNFNHF |
| | | transporter | | | VVLKEVNARGVVVHDPGHGLCKLTL |
| | | | | | DEVSAAFTGVALELWPENDFQPAAA |
| | | | | | QPPIPLRKLLGRVQGFGRVLTQVLL |
| | | | | | LALALELCTILSPFFMQTVIDKVLV |
| | | | | | SADLDLLTVLAIGFGLLLIIQHTVA |
| | | | | | LARAWALMYLGTMLGSQWQINVFTH |
| | | | | | LLRLPVAFFERRHLGDIVSRFGSLK |
| | | | | | AIQQTVTTSFIEALLDGLMTVVTLG |
| | | | | | LMIAYSPKLALVAVVAMIIYGLARW |
| | | | | | IWFGPLRRASENQLVHAARQQSHFL |
| | | | | | ESARGVRTIKLFNRHEQRCSAWSAL |
| | | | | | LVEEINAGLHPQKLQLAYRAFNGVL |
| | | | | | FGLVTIIVIWMGARLVLEGQFSAGM |
| | | | | | LIAFNSYKEQFNGRVAGLIDKIVDV |
| | | | | | IMLRLHGERLADIVLHDAEPIAGPD |
| | | | | | TFDDACNDRVPSLEVRQLRFRYSEH |
| | | | | | EPYVLDDVSIHIHAGESVAIVGPSG |
| | | | | | GGKSTLLNVMLGILAPCAGSVLLDG |
| | | | | | LPVGPENIGRLRRITGTVLQDDVLF |
| | | | | | AGSIGDNISFFETGADQKWIEQCAH |
| | | | | | LAAVHDDIAQMPMGYNTLVGDMGTV |
| | | | | | LSGGQKQRVLLARALYKRPQLLFLD |
| | | | | | EATSHLDINREAAVNQALQTLNITR |
| | | | | | IIVAHRPETIRTADRVVALAQGRVA |
| | | | | | YDGPVLNEAPSA |
| 2 | WP_036996087.1 | peptidase | Pseudomonas | 83 | MNIRQSLAFGIGRKLPMVLQAEAAE |
| | | domain- | donghuensis | | CGQACLAMIAGYHGQHHDLFSLRQK |
| | | containing | | | LSPSMKGATLKQLMAMAAQLGLASR |
| | | ABC | | | PLRLELQALGQLRLPCVLHWNFNHF |
| | | transporter | | | VVLKEVGPRGVTVHDPGRGVCKLSF |
| | | | | | DEVSTAFTGVALELWPQGDFQPGPA |
| | | | | | NPPLRLRQLLGRVQGFAGVLSHVLL |
| | | | | | LAAALELCIVLSPFFLQTVIDKVLV |
| | | | | | SADLDLLAVLAIGFGLLLLMQQTLA |
| | | | | | LARSWALMYLGTLLSSQWQINVFSH |
| | | | | | LVRLPVAFFERRHLGDIVSRFGSLK |
| | | | | | SIQHTLTTSFVEALLDGLMTLVTLG |
| | | | | | LMFAYSPLLALIALLAMVIYALARW |
| | | | | | AWFGPLRRASEEQLVHAARQQSYFL |
| | | | | | ESMRGVRTIKLFGHQEQRASTWGSL |
| | | | | | LVEEINAGLRPQKLELAYRAFNGLL |
| | | | | | FGLVTLLVIWLGARLVLEGQFSAGM |
| | | | | | LIAFNAYKEQFNSRVAGLIDKLVDV |
| | | | | | LMLRLHGERLADILLQVPEHRQFPA |
| | | | | | AGDSDADECVPSLEVRQLKFRYGEH |
| | | | | | EPYVLDEVSVRIEAGDSLAIVGPSG |
| | | | | | GGKTTLLNVMLGILTPSSGSVLLDG |
| | | | | | VAIDSHNLDRLRRISATVLQDDVLF |
| | | | | | AGSIADNISFFSAEANPRWVEQCAR |
| | | | | | MAAVHDDIAQMPMAYNTLVGDMGTV |
| | | | | | LSGGQKQRILLARALYRRPKLLFLD |
| | | | | | EATSHLDIAREAAVNQALQALNITR |
| | | | | | IIVAHRPDTIRSARRVLALDNGRVV |
| | | | | | YDGPLIETDEHAPAESGEIIRGAAS |
| | | | | | SS |
| 2 | WP_062574732.1 | MULTISPECIES: | Pseudomonas | 84 | MNYIDAITPSLGRRLPLILQTEATE |
| | | peptidase | putida | | CGLACLAMIAGMHGHHTTLMELRRQ |
| | | domain- | group | | FSVSLKGITLRQLMQTAERLNLGTR |
| | | containing | | | AVRLELSDLNKLKLPCVLHWNFNHF |
| | | ABC | | | VVLKAVHGQHLTLHDPGQGVRQLSF |
| | | transporter | | | AQASECFTGVALEVWPEQGFAQQAA |
| | | | | | KPRLGLTKMLGRVSGLYRGLAQVLL |
| | | | | | LALVLEVFSLVSPFLLQWTLDNVVV |
| | | | | | TQDRDLLTTLMIGFGLLLLMQQLVS |
| | | | | | ATRAWVMMHMSTLLSVQWRANLFTH |
| | | | | | LLRLPIQYFEKRHLGDVVSRFGAVD |
| | | | | | QIQHTLTAAFFSAILDGLMTVATLV |
| | | | | | LMFLYSPSLAMIAVLAMVAYILVRW |
| | | | | | AWYGPLRRASEEQIVHAARQQSHFL |
| | | | | | ETLRGVRTLKLFQRQGERRSTWLGL |
| | | | | | LVAQINAGLRTQKLQLLYTQLNALI |
| | | | | | FGLENLCTIGLGASLVMDNHFTVGV |
| | | | | | LMAFMAYKGQFVSRVASLVDHLFEL |
| | | | | | HMLRLQGERLADIVMHPPEEQRDAP |
| | | | | | QSVDWPARQMTIDIQGLSYRYSEHE |
| | | | | | PLVLDGIDLRIAPGESVAITGASGC |
| | | | | | GKSTLVNVLLGILPPTSGQIRMGGH |
| | | | | | ELRQLDMASWRGRIGTVMQDDCLFA |
| | | | | | GSLADNITFFESSPDLEWAMECARM |
| | | | | | AAIHAEIMAMPMGYNTLVGDMGTVL |
| | | | | | SGGQKQRLLLARALYKRPDILILDE |
| | | | | | ATSHLDAASEQRVNAAVRALAVTRI |
| | | | | | IVAHRAETIASADRVVRLEAGRMVL |
| | | | | | DQRVASVSSYLDEAVTP |
| 2 | WP_173179306.1 | ATP- | Pseudomonas | 85 | MAQLRFDSRSKLPIIMQQEKSECGF |
| | | binding | sp. | | ACIAMIASFYGHEISLREVRAMHRG |
| | | cassette | TUM18999 | | SSRGATLLEIVKVAMQLGMQARPLK |
| | | domain- | | | IENNEIDSLKLPALLHWKNSHYVVL |
| | | containing | | | RELRAGKFCIHDPASGVVNLSREEF |
| | | protein | | | SIFFTGAALELAPGHQFQLKNKIPG |
| | | | | | VAVRDILGKVHGLHEALVGILASAL |
| | | | | | ALEAFSFITPLFTQLVIDKAISSAD |
| | | | | | LDLLLVFGMSFCVLIIFQSFISYLR |
| | | | | | DWLVCRTGSTLNQSWTGGVFAHLMS |
| | | | | | LPEDYFQRRSLGDIGSRFGSLKNIQ |
| | | | | | QAITSTITTSFLDGFMSLVTLAAMV |
| | | | | | IYSPKLATVAFVFTSAYVLVRLALY |
| | | | | | SKFKSLNIDLISAQARQNSKFYEAV |
| | | | | | RASQTIKANNLTAQVTTTYLNLVTS |
| | | | | | YLNRNILASRLTIAFKAFADLIQGI |
| | | | | | EKIVLLWIGATLVIQQELTAGMLMS |
| | | | | | ISMFSLLFGSKIAHLADNYVELKLL |
| | | | | | KIHAARISDIVHTEKELHYYPTFFG |
| | | | | | EIKDKTIEFASVYFRYSPSEPWILE |
| | | | | | NCCFTIKPGEAVAITGSSGIGKSTI |
| | | | | | LKLICGLLEPNSGKILIGGVDVTTI |
| | | | | | GKEQLRSWMAVILQDDTLFSGTIAE |
| | | | | | NISLFNEQYSIEEISTAAQEACIHG |
| | | | | | EIIKMPMGYNTLIGDMGSSLSGGQR |
| | | | | | QRLLIARSLIRTPSFLLFDEATSHL |
| | | | | | DIENEFRISEILAKRNCTRIIVAHR |
| | | | | | EATIKLCNRILHMEGRRLKDKTNKT |
| | | | | | ITPHLVV |
| 2 | WP_080480033.1 | peptidase | Pseudomonas | 86 | MNLKNAFTFGVGTKLPVVLQTEATE |
| | | domain- | syringae | | CGLACMAMIARYHGQHSDLFSLRKR |
| | | containing | | | LSPSMKGVNLKQLISMAAQLGLGSR |
| | | ABC | | | ALRVELSALGQLQLPCVLHWNFNHF |
| | | transporter | | | VVLREVTAYGVVLHDPARGLCKLSL |
| | | | | | DEVSAAFTGVALEVWPESDFQPVAA |
| | | | | | RPPISLRKLLGRVHGFGRVLTHVLL |
| | | | | | LALALELCTILSPFFMQTVIDKVLV |
| | | | | | SADLDLLAVLAIGFGILLIMQHTVA |
| | | | | | LARGWALMYLSTMLGAQWQINVFTH |
| | | | | | LLRLPVAFFERRHLGDIVSRFGSLK |
| | | | | | SIQQTVTTSFIEAILDGLMTVITLG |
| | | | | | LMIAYSPKLALVAVVAMVIYALARW |
| | | | | | AWFGPLRRASENQLVHAARQQSHFL |
| | | | | | ESVRGVRTIKLFNRPEQRCSAWSAL |
| | | | | | LVEEINAGLHPQKLQLAYRTFNGVL |
| | | | | | FGLVTIIVIWLGATLVLEGQFSAGM |
| | | | | | LIAFNSYKEQFNGRVAALIDKIVDV |
| | | | | | IMLRLHGERLADIVLQDAEPVSGLD |
| | | | | | GSDDASSENVPSLEVRDVRFRYSEY |
| | | | | | EPYVLDGVSIHVRAGESVAIVGPSG |
| | | | | | GGKSTLLNVMLGIMAPSAGSVLLDG |
| | | | | | QPVGPDHVSRLRRLTGTVLQDDVLF |
| | | | | | AGSIGDNISFFETSADQQWIEQCAR |
| | | | | | LAAVHDDIAQMPMGYNTLVGDMGTV |
| | | | | | LSGGQKQRVLLARALYKRPQLLFLD |
| | | | | | EATSHLDITREAAVNQALETLNITR |
| | | | | | IIVAHRPETIRSADRVVALANGRIA |
| | | | | | YDGPVLGEPSAQGPEP |
| 2 | WP_060404286.1 | MULTISPECIES: | Pseudomonas | 87 | MNLRNTFAFGVGRKLPVVLQAEATE |
| | | peptidase | syringae | | CGLACMAMITSYHGQHSDLLSLRQR |
| | | domain- | group | | LSPSMKGVNLKQLTHMAARLGLGSR |
| | | containing | | | ALRVELNALGQLQLPCVLHWNFNHF |
| | | ABC | | | VVLKEVNARGVVVHDPGRGVCKLTL |
| | | transporter | | | DEVSAAFTGVALELWPESDFQPAAA |
| | | | | | QPPIPLRKLLGRVQGFGRVLTQVLL |
| | | | | | LALALELCTILSPFFMQTVIDKVLV |
| | | | | | SADLDLLTVLAIGFGLLLIIQHTVA |
| | | | | | LARAWALMYLGTMLGSQWQINVFTH |
| | | | | | LLRLPVAFFERRHLGDIVSRFGSLK |
| | | | | | AIQQTVTTSFIEALLDGLMTVVTLG |
| | | | | | LMIAYSPKLALVAVVAMIIYGLARW |
| | | | | | IWFGPLRRASENQLVHAARQQSHFL |
| | | | | | ESARGVRTIKLFNRHEQRCSAWSAL |
| | | | | | LVEEINAGLHPQKLQLAYRAFNGVL |
| | | | | | FGLVTIIVIWMGARLVLEGQFSAGM |
| | | | | | LIAFNSYKEQFNGRVAGLIDKIVDV |
| | | | | | IMLRLHGERLADIVLHDVEPIAGPD |
| | | | | | TFDDACNDRVPSLEVRQLRFRYSEH |
| | | | | | EPYVLDDVSIHIRAGESVAIVGPSG |
| | | | | | GGKSTLLNVMLGILAPCAGSVLLDG |
| | | | | | LPVGPENIGRLRRITGTVLQDDVLF |
| | | | | | AGSIGDNISFFETGADQKWIEQCAH |
| | | | | | LAAVHDDIAQMPMGYNTLVGDMGTV |
| | | | | | LSGGQRQRVLLARALYKRPQLLFLD |
| | | | | | EATSHLDINREAAVNQALQTLNITR |
| | | | | | IIVAHRPETIRTADRVVALAQGRVA |
| | | | | | YDGPVLNEAPSA |
| 2 | WP_010196175.1 | MULTISPECIES: | Pseudomonas | 88 | MNLRNTFAFGVGRKLPVVLQAEATE |
| | | peptidase | syringae | | CGLACMVMIASYHGQHSDLLSLRQR |
| | | domain- | group | | LSPSMKGVNLKQLTHMAARLGLGSR |
| | | containing | genomo | | ALRVELNALGQLQLPCVLHWNFNHF |
| | | ABC | sp. 2 | | VVLKEVNARGVVVHDPGRGLCKLTL |
| | | transporter | | | DEVSAAFTGVALELWPESDFQPAAA |
| | | | | | QPPIPLRKLLGRVQGFGRVLTQVLL |
| | | | | | LALALELCTILSPFFMQTVIDKVLV |
| | | | | | SADLDLLTVLAIGFGLLLIIQHTVA |
| | | | | | LARAWALMYLGTMLGSQWQINVFTH |
| | | | | | LLRLPVAFFERRHLGDIVSRFGSLK |
| | | | | | AIQQTVTTSFIEALLDGLMTVVTLG |
| | | | | | LMIAYSPKLALVAVVAMIIYGLARW |
| | | | | | IWFGPLRRASENQLVHAARQQSHFL |
| | | | | | ESARGVRTIKLFNRHEQRCSAWSAL |
| | | | | | LVEEINAGLHPQKLQLAYRAFNGVL |
| | | | | | FGLVTIIVIWMGARLVLEGQFSAGM |
| | | | | | LIAFNSYKEQFNGRVAGLIDKIVDV |
| | | | | | IMLRLHGERLADIVLHDAEPIAGPD |
| | | | | | TFDDACNDRVPSLEVRQLRFRYSEH |
| | | | | | EPYVLDDVSIHIRAGESVAIVGPSG |
| | | | | | GGKSTLLNVMLGILAPCAGSVLLDG |
| | | | | | LPVGPENIGRLRRITGTVLQDDVLF |
| | | | | | AGSIGDNISFFETGADQKWIEQCAH |
| | | | | | LAAVHDDIAQMPMGYNTLVGDMGTV |
| | | | | | LSGGQKQRVLLARALYKRPQLLFLD |
| | | | | | EATSHLDINREAAVNQALQTLNITR |
| | | | | | IIVAHRPETIRTADRVVALAQGRVA |
| | | | | | YDGPVLNEAPSA |
| 2 | WP_007251627.1 | peptidase | Pseudomonas | 89 | MNLKNAFTFGVGRKLPVVLQAEATE |
| | | domain- | syringae | | CGLACMAMIAGYHGQHSDLFSLRER |
| | | containing | group | | LSPSMKGVNLKQLTRMAAQLGLGSR |
| | | ABC | genomosp. 3 | | ALRVELSALGQLQLPCVLHWNFNHF |
| | | transporter | | | VVLKEVTAQGVVVHDPARGLCKLSL |
| | | | | | DEVSAAFTGVALEVWPESDFQPAAA |
| | | | | | RPPIPLRKLLGRVQGFGRVLTHVLL |
| | | | | | LALALELCAILSPFFMQTVIDKVLV |
| | | | | | SADLDLLAVLAIGFGILLIIQHTVA |
| | | | | | LARGWALMYLGTMLGSQWQINVFTH |
| | | | | | LLRLPVAFFERRHLGDIVSRFGSLK |
| | | | | | SIQQTVTTSFIEAILDGLMTVITLG |
| | | | | | LMIAYSPKLALVAVVAMVIYALARW |
| | | | | | AWFGPLRRASENQLVHAARQQSHFL |
| | | | | | ESVRGVRTIKLFNRHEQRCSAWSAL |
| | | | | | LVEEINAGLHPQKLQLAYRTFNGVL |
| | | | | | FGLVTIIVIWLGARLVLEGQFSAGM |
| | | | | | LIAFNSYKEQFNSRVAGLIDKIVDV |
| | | | | | IMLRLHGERLADIVLQEAEPVSGLD |
| | | | | | AFDEASIEKVPSLEVRDLRFRYSEH |
| | | | | | EPYVLDGVSIHVRAGESVAIVGPSG |
| | | | | | GGKSTLLNVMLGILAPSAGSVLLDG |
| | | | | | LPVGPEHVSRLRRLTGTVLQDDVLF |
| | | | | | AGSIGDNISFFETDADQQWIEQCAR |
| | | | | | LAAVHDDIAQMPMGYNTLVGDMGTV |
| | | | | | LSGGQKQRVLLARALYKKPQLLFLD |
| | | | | | EATSHLDIAREAAVNQALQALNITR |
| | | | | | IIVAHRPETIRSADRVVALANGRIA |
| | | | | | YDGPVLDEPSARGSEP |
| 2 | WP_032686861.1 | peptidase | Raoultella | 90 | MDLFHKLNLSFRHKLPVLRQTQAAE |
| | | domain- | planticola | | CGLTCVGMIAGYYGYQIDMVSLRHR |
| | | containing | | | FPSSQKGSTLADVMSFTQKLDMGCR |
| | | ABC | | | AVRLELDELNKLKLPCILHWDMNHF |
| | | transporter | | | VVLKSINKNTLTIHDPARGARKVAM |
| | | | | | DEVSRSFTGVALELYPAATFERKDE |
| | | | | | KKSISMLSLIRNVSGIGSAFIQVAL |
| | | | | | LSLALEFFGIITPFYMQWVIDQVLV |
| | | | | | SADRDLLTLLGVAFIFITLFQNLIG |
| | | | | | ALRSWVTTWFSSMLSVQWSSNLCAH |
| | | | | | LLGLPLSYFEDRHVGDILSRFGSIA |
| | | | | | NIQSTLTGRFISSIFDGIMALVTLG |
| | | | | | VIFAYNTSLTFIVIGLFLLYALIRW |
| | | | | | ISFEPFRQASEDQLLASAQAQSQLL |
| | | | | | ESIRGVQAIKLNNKQDLRVSTFTNE |
| | | | | | TVQSTNKGITTQKLSIGFSTLQGTI |
| | | | | | SGVGKIVLIWLAAAQVLDGNFTSGM |
| | | | | | LVAFISFSDQFISRSAGLINALIDF |
| | | | | | KMLRLHGERLADIVLSEKEPDLASS |
| | | | | | ASLPDREGPVHLAIHQLSFRYSATD |
| | | | | | GEIFTGFNLEIEAGESIAIIGPSGQ |
| | | | | | GKTTLAKLLLGLLKPENGSICINGI |
| | | | | | DHRKLGMTLYRDLIGSVMQTDQLFA |
| | | | | | GSIIDNISFFDASLDRLRVERVARI |
| | | | | | AQIHDDIMAMPMGYNSMVGDMGSSL |
| | | | | | SGGQIQRVILARALYRNPRLLILDE |
| | | | | | ATSHLDIARESAINDAIRHMNVTRI |
| | | | | | IIAHRPETILSADRIIEIHSRGVIE |
| | | | | | VAKHDFIHAIKNSQSVLRV |
| 2 | WP_032695324.1 | peptidase | Raoultella | 91 | MDLFHKLNLSFRHKLPVLRQTQAAE |
| | | domain- | planticola | | CGLTCVGMIAGYYGYQIDMVSLRHR |
| | | containing | | | FPSSQKGSTLADVMSFTQKLDMGCR |
| | | ABC | | | AVRLELDELNKLKLPCILHWDMNHF |
| | | transporter | | | VVLKSINKNTLTIHDPARGARKVAM |
| | | | | | DEVSRSFTGVALELYPAATFERKDE |
| | | | | | KKSISMLSLIRNVSGIGSAFIQVAL |
| | | | | | LSLALEFFGIITPFYMQWVIDQVLV |
| | | | | | SADRDLLTLLGVAFIFITLFQNLIG |
| | | | | | ALRSWVTTWFSSMLSVQWSSNLCAH |
| | | | | | LLGLPLSYFEDRHVGDILSRFGSIA |
| | | | | | NIQSTLTGRFISSIFDGIMALVTLG |
| | | | | | VIFAYNTSLTFIVIGLFLLYALIRW |
| | | | | | ISFEPFRQASEDQLLASAQAQSQLL |
| | | | | | ESIRGVQAIKLNNKQDLRVSTFTNE |
| | | | | | TVQSTNKGITTQKLSIGFSTLQGTI |
| | | | | | SGVGKIVLIWLAAAQVLDGNFTSGM |
| | | | | | LVAFISFSDQFISRSAGLINALIDF |
| | | | | | KMLRLHGERLADIVLSEKEPDLASS |
| | | | | | ASLPDREGPVHLAIHQLSFRYSATD |
| | | | | | GEIFTGFNLEIEAGESIAIIGPSGQ |
| | | | | | GKTTLAKLLLGLLKPENGSICINGI |
| | | | | | DHRKLGMTLYRDLIGSVMQTDQLFA |
| | | | | | GSIIDNISFFDASLDRLRVERVARI |
| | | | | | AQIHDDIMAMPMGYNSMVGDMGSSL |
| | | | | | SGGQIQRVILARALYRNPRLLILDE |
| | | | | | ATSHLDIARESAINDAIRHMNVTRI |
| | | | | | IIAHRPETILSADRIIEIHSRGVIE |
| | | | | | VAKHDFIHAIKNSQSVLQV |
| 2 | WP_063918175.1 | MULTISPECIES: | Serratia | 92 | MVFSWQKTPLILQSEMNECGLACLA |
| | | peptidase | | | MMAGYFGKRIDLASARTLHGASSHG |
| | | domain- | | | MTLRDLITAFERVGMTARASRVELD |
| | | containing | | | ELRSLTRPAILHWSFNHFVVLVKVT |
| | | ABC | | | RRGAVILDPAIGRRSISLRELSDKF |
| | | transporter | | | TGILMEAWPAETFDKKPLEMNVTVS |
| | | | | | DLFRGVRGLRRIFTGVLMLSVLVEL |
| | | | | | LSIAVPAASQFTIDTLVRSSDREGI |
| | | | | | FFVGIVVIAALLIKSAFSVVRAWIL |
| | | | | | MNLRYTLGVKWAEMFFNRLIRLTLS |
| | | | | | FFEKRHTGDIASRFQSLTAIQEAFT |
| | | | | | ADMVASLLDAIVIVISMAIIFTYSP |
| | | | | | VLAIGPLIAACAYAALKAGLFSTYR |
| | | | | | NRKIEHIAFEAVQSSHFLETVRAIS |
| | | | | | AIKMLNLTPVRRREWVNHVVNSTHA |
| | | | | | GNQLFKLDLLTNTAAVLLVGFSGIY |
| | | | | | VLSVGAIGFDKGITTGALLAVMLYA |
| | | | | | DMVITRTVKLVNAVSDFCLVSMHSQ |
| | | | | | RLTDVAVSPVERDEGEHVSPQLNGH |
| | | | | | IVIRNLAFRHSQAERNIFEGINLEV |
| | | | | | MPGENVAIVGPSGCGKSTFLHVLAG |
| | | | | | LYESTEGDVFINNVGMSGMGKRDIR |
| | | | | | EHVAFVMQDDKLLAGTIQQNITGFT |
| | | | | | ASPDMERMAECANHASIDEEISAFP |
| | | | | | QGYDSMIGDIGSTLSGGQRQRISIA |
| | | | | | RALYRQPHVLLLDEATSDLDIDNEK |
| | | | | | RITRAIGQLPITRIFVAHRPEMIKS |
| | | | | | ADRVFNLHLNAWVKQESRGTATMLI |
| | | | | | ADKVHSS |
| 2 | WP_006316838.1 | peptidase | Serratia inhibens | 93 | MKFLESLNFGWRRRLPVIQQTQSAE |
| | | domain- | | | CGLACIGMIANFYGHSIDMVSLRRR |
| | | containing | | | FSTSLKGATLADIMLMANRLDMAGR |
| | | ABC | | | ALRLDLDELAKLRRPCILHWELNHF |
| | | transporter | | | VVLKSVSKDKITIHDPARGKRDVPM |
| | | | | | DEVSRCFTGVALELLPSATFKKVSE |
| | | | | | KESISMLKLIGNVTGIGSAFAQIMI |
| | | | | | LSVALEVFGILSPFYMQWVLDQVLV |
| | | | | | SADYDLLTLLGTGFIIIMILQNIIA |
| | | | | | ALRSWVTTWFSSLLSVQWTTNVCSH |
| | | | | | LLGLPMSYFEERHIGDVVSRFGSIS |
| | | | | | TIQSTLTGRFISSILDGVMAIVTLG |
| | | | | | MLFAYSSSLTWLVLGLFLAYMIIRW |
| | | | | | VAFRPFRQANEDQIVASARAQSQLL |
| | | | | | ESIRGVQAVKLNNKQETRIAAYANA |
| | | | | | LVEATNKGIAIERLSIGFSTLQGTI |
| | | | | | SGVGRVALVWLAATQVLEGNFTSGM |
| | | | | | LVAYVSFSDQFMSRASGLVDAVIEF |
| | | | | | TMLRLHGERLADIVLTDTEADMENR |
| | | | | | VALPATLSDRSAPAVDIENLCFRYS |
| | | | | | ETEPLVINHCNFSIRSGESVAIIGP |
| | | | | | SGQGKTTLAKLLLGLLKPESGSITI |
| | | | | | DGLDIRKMGMHHYRDRIGCVMQDDI |
| | | | | | LFAGSIADNICFFDSVPNQALIEKS |
| | | | | | ATIAQIHSDIMAMPMGYHSLVGDMG |
| | | | | | SSLSGGQIQRVLLARALYRQPDLLI |
| | | | | | LDEASSHLDIERERFINESIKSMPM |
| | | | | | TRVIIAHRPETIRSADRVILLSQGV |
| | | | | | AQEIEKDDFMTVFESEDISNN |
| 2 | WP_004952388.1 | peptidase | Serratia | 94 | MDFSHKLNLSFRQKLPLLRQTQAAE |
| | | domain- | plymuthica | | CGLSCVGMIAGYYGHSIDMVTLRQR |
| | | containing | | | FPTSLKGSTLSDVMSIAQNMGMSCR |
| | | ABC | | | AVRLELSELQKLSLPCVLHWDMNHF |
| | | transporter | | | VVLKSIAGNQVVIHDPARGIRKVPM |
| | | | | | EEVSRSFTGIALELYPVASFEIRDE |
| | | | | | KKSISMLSLIRNVSGIGSAFWQVAL |
| | | | | | LSLVLEFFGIISPFYMQWVIDQVLV |
| | | | | | SADHELLTLLGVAFIGITLFQNIIS |
| | | | | | ALRAWVTTWFSSMLSVQWSSNLCTH |
| | | | | | LLGLPLSYFEERHVGDILSRFGSIS |
| | | | | | NIQNTLTGSFISSIFDGIMSLVTLM |
| | | | | | VIFTYNATLSFVVIGLFLSYAVIRW |
| | | | | | VSFEPFRQANEDHLMAAALAQSQLL |
| | | | | | ESIRGVQAIKLNNKQDLRVSAYANE |
| | | | | | MVEAANKGITIQKLSIGFNTLQSII |
| | | | | | SGIGKILLIWLAAVQVLEGNFTSGM |
| | | | | | LIAFITFSDQFLSRSSGLINALIEF |
| | | | | | KMLRLHGERIADIVLTEKEKNMESK |
| | | | | | VTLPEDNGPVRLDVRALSFRYSSTD |
| | | | | | TAIFSDFNLSIQAGESVAIIGPSGQ |
| | | | | | GKTTLAKLLLGLLKPESGTICINDI |
| | | | | | DHTKLGMSLYRDLIGSVMQSDMLFA |
| | | | | | GSIMDNINFFDANLDRRHVERVARI |
| | | | | | AQIHDDIMAMPMGYNSMVGDMGSSL |
| | | | | | SGGQIQRVILARALYRNPRILILDE |
| | | | | | ATSHLDVARESAINNAIKHMDMTRI |
| | | | | | LIAHRPETILSADRIIQINRHGAME |
| | | | | | IEKSDFIRLVNASQPILHAS |
| 2 | WP_053093500.1 | peptidase | Stenotrophomonas | 95 | MADGSASAERRRPDRHSRADVRAEL |
| | | domain- | maltophilia | | RFGWRRDTPLILQAETAECGLACLA |
| | | containing | | | MVAGHHGHHVDMLGMRAKFGSSQKG |
| | | ABC | | | VTLRQMMHLATALGMEGRPLKLDLG |
| | | transporter | | | NLSRLKLPCILHWGMNHFVVLCKIG |
| | | | | | PRSAVIHDPARGVRKLDLLEVSSQF |
| | | | | | TGVALELIPGADFRPAHRSPSISLR |
| | | | | | RLAGTIQGLVPACLQVLGLALALEA |
| | | | | | FILIGPFYLQWTMDQVIVSADRDLL |
| | | | | | SLLAIGFAIVAVFQVVITALRAWLL |
| | | | | | SWISATTSAQWMSNMVGHLLRLPLA |
| | | | | | WFEKRQVGDIVSRLGSVQIIQSTLT |
| | | | | | VQFIGSLLDGLMSLFVLLIMTFYSP |
| | | | | | GLAALVAGLFLLYTMLRWVFFAPLW |
| | | | | | RANEDQIVHAARQQGEILESIRGIL |
| | | | | | PIKLANQQNVRRARYANASVQVINR |
| | | | | | GIRIQQLNISFGIINGLIFGLGRVA |
| | | | | | LIWIAASLVMDEKFSVGMMVAFVAF |
| | | | | | ADMFIHRSTSLADKWVDFRMLRLHA |
| | | | | | ERLSDIALSEQESPDGVDSCHVPDD |
| | | | | | SSVEFKNVSFRYSDGEPWVLRNCSF |
| | | | | | KVESGRSIAITGVSGSGKTTLAKIM |
| | | | | | LGLLKPSEGEVIFGGQKIDDMGHGR |
| | | | | | FRGLVGAVMQEDQLFAGTIGDNISF |
| | | | | | GDQDSTDERIREAATLAAVSDDIEA |
| | | | | | MPMGYRTLVGDMGSSLSGGQKQRVI |
| | | | | | LARALYRRPRLLVLDEATSHLDLEC |
| | | | | | ERRVNAAVKMSSMTRVVIAHRLETI |
| | | | | | QSADERFEIKHREC |
| 2 | WP_014089914.1 | MULTISPECIES: | Xanthomonas | 96 | MKDVLTPSADGDAEPDRSAEPSLVF |
| | | peptidase | | | SSKRRVPHIRQTESSECGLACIAML |
| | | domain- | | | LSYYGHETSLGELRNRFTVSTSGAT |
| | | containing | | | LASLIEIADANGLTTRPLRLELYEL |
| | | ABC | | | PKLSLPCMVHLHHGHFVVLTAVRGG |
| | | transporter | | | HVHISDPAAGVKKIKLDEFDELFSG |
| | | | | | IALEAHPGPAFKKIKSVPSVSLRDL |
| | | | | | AGSLRSLVPAFWGVGILALFLEGCA |
| | | | | | LIAPQYLRLTMDQVLSVKDDGLVTT |
| | | | | | LAIGFSIVLLVQLVLTVARKWTLLW |
| | | | | | ISSTTSLRWSSNLFRHLVSLSQSYF |
| | | | | | VKRHTGDISSRFQSIYSIQQSLTTK |
| | | | | | MMEAVIDGVMSFLMLLLLMSYDAPL |
| | | | | | AFALLAFTIAYVGSRYAYYAKLKEA |
| | | | | | NLDQINIDARRQSLLYETVRSIQPI |
| | | | | | KLFNKSALWASRFTNYSAKATNATI |
| | | | | | GAERIRIGFDSVQLLIQGLCRIFVI |
| | | | | | WEGTRLVLSGDMTVGVLTVFLIYAT |
| | | | | | QFSQRSINLADYLMQLRLLRLHTER |
| | | | | | ISDITHSEPESFLHGNGALEDAAPT |
| | | | | | IAIANGYFRYSSTDKWIMSALQLRA |
| | | | | | PAGQVIAIVGNSGVGKTTLIRVLAG |
| | | | | | LEDLQVGDFLVNGEDLRKVGKSSYR |
| | | | | | SKVSIVMQGDNLLSGTLSENISMFD |
| | | | | | EHIDQERLVQAAKLACIHDDIQRMP |
| | | | | | MGFNTRVGDLGNTLSGGQKQRVFLA |
| | | | | | RAFYRRTKLLLMDEPTTGIDEQMGI |
| | | | | | QLMKNIKSIGATTVIVTHDKNISRM |
| | | | | | CDMHYLFVGGALRPLIKEQPSKFER |
| | | | | | DEAGETAEKSSQQ |
| 2 | WP_016849823.1 | MULTISPECIES: | Xanthomonas | 97 | MTMADGSPTLLWSQKKRVDPIMQSE |
| | | peptidase | | | AAECALVCLAMLAQFYGNDCGLPAL |
| | | domain- | | | RRRYSSTLKGMSLNQVIEVADDMGF |
| | | containing | | | DCRALRAELDYLREVQAPVILHWNM |
| | | ABC | | | NHFVVLAGTKGSKLVIIDPAQGRRL |
| | | transporter | | | MPMGEVSKYFTGVLLELEPSASFRR |
| | | | | | ASKSPTVPLSALTGRISGLKRVLIQ |
| | | | | | IFALALAIEALGLSIPLQAQWAVDQ |
| | | | | | ISESNGKVILTVAAIFSLVVIIQAG |
| | | | | | LNLARAWLISWLGANISTQWVINIF |
| | | | | | SHLMRLPLDFFEKRHLGDIMSRFGS |
| | | | | | IHWIQAILTGSFVTALLDGLTGSLA |
| | | | | | LVLLFIYSTGMATVAVAVATLYALG |
| | | | | | RFLMFGALWRASEQSMVFDARQQSE |
| | | | | | LMESVRGIQAIKLANRQLERRARLA |
| | | | | | NATLEAAKRGITAERIKLGFGAASQ |
| | | | | | GIFGLQRVFLLSAGAYLISRNAMTA |
| | | | | | GMMVAALSYADQFSIRVGSLIDNAV |
| | | | | | ELKMLGLHLNRLADITTADEEPARG |
| | | | | | RLEGALRSAPEVQVKGLGYRYSEWE |
| | | | | | PWIFRNLSFTIAPGQSVALVGPSGC |
| | | | | | GKTTLAKIVLGLIAPQEGEVTVTDQ |
| | | | | | PSPVCRSAIPAEGMAAVMQDDCLFS |
| | | | | | GTIADNVAFFDSSAELSKIEAAARA |
| | | | | | AGIHDDIVKMPMGYETLVGDMGSTL |
| | | | | | SGGQKQRILLARALYTQPKILVLDE |
| | | | | | ATSHLDSKNEKVVNDAVMDLEITRI |
| | | | | | IIAHREQTIAMADRVFDLSRQTWIR |
| 2 | WP_039573748.1 | MULTISPECIES: | Xanthomonas | 98 | MSRWQAQLRRASGVRGLPMILQGQV |
| | | peptidase | | | GECGLAAMAMIAHYHGCHIGLAELR |
| | | domain- | | | RRFLLSRQGTNLANLVAIAQALGLQ |
| | | containing | | | ARALRLEMDALPELQLPCIVHWDLN |
| | | ABC | | | NFVVLKRVGARRLQIHDPASGPRSL |
| | | transporter | | | TPGEFARHFSGIALELSPAADFRPQ |
| | | | | | AAAPAVALSSLIGRVHGLGRAVWQV |
| | | | | | LALAFALEVLSLGMPFQLQWIVDQA |
| | | | | | VPSADIGLIHVLGAGFLLLVVLQSC |
| | | | | | IALLRGWLIASVSAQLGFQWMGQVF |
| | | | | | AHLLALPLAYFEKRHLGGIQSRFAS |
| | | | | | IVQVQRTLTTGFTQTLVDGVLVIGT |
| | | | | | LGLMLVYSVGLSAITLVAVALYAAT |
| | | | | | RWLWLRRMREAAAEQLLWDARQHTH |
| | | | | | LLESVRGIQGVRLFGRQQVRRMDWT |
| | | | | | HLLAEQTNAQLRLAQGEVWQSSLQR |
| | | | | | LLFGCERVLVIWLAAFAILRAELSL |
| | | | | | GMLLAFMAYREQFAMRLSELIDRLV |
| | | | | | EFRLLRVHLERVADIVHQPREEADQ |
| | | | | | RIDAPAWNDTTIELCGIGFRYADDT |
| | | | | | PAVLEDVSVRIASGECVAITGASGC |
| | | | | | GKTTLVKVILGLLQPSTGQVKIGGR |
| | | | | | PLSLAALAPYRAIVGTVMQDDLLFT |
| | | | | | GSVSENISFFDPEPDQAQIERCARI |
| | | | | | AGVHQEVEQMPLGYASLLSEAGTGL |
| | | | | | SGGQRQRVLLARALYRSPRILVLDE |
| | | | | | ATSHLDVMNEQRVNRAIQAMQVTRI |
| | | | | | IVAHRSETVAMAARVITLEQGRVVS |
| | | | | | DQPIADWQRLQERSTAAD |
| 2 | WP_099802272.1 | MULTISPECIES: | Xanthomonas | 99 | MKSIIQSEAAECGLVALAITAASHG |
| | | peptidase | | | LNIGLPEMRRRFPLSLKGAKLNHLI |
| | | domain- | | | RIAQQLGFATRPLRLDMEHLSKLKL |
| | | containing | | | PCVLHWDLNHYVVLAKVGKLKATIF |
| | | ABC | | | DPALGERTLLMDEVSKHFTGVALEL |
| | | transporter | | | TPTAEFKPKKATSSVSVRQLTGSIR |
| | | | | | GLGGAVLQILLLSFALQVFVILAPF |
| | | | | | YMQWVVDQVLVSGDHALLMVVGLGF |
| | | | | | GLALLLQIGIGLLRGWSVVYLSSTL |
| | | | | | GLQWMTNVFAHLLKLPLFFFEKRHL |
| | | | | | GDVTSRLSSVQTIQNTLTTSFVESI |
| | | | | | IDGLMTIVTLGLMLIYSWKLALVTL |
| | | | | | LAVGLYLAIRAIAFHPMRNCTELQL |
| | | | | | VAAAKQQTHLLESLRGIQSLKVAGE |
| | | | | | ESQRRSTYEGLLNDTVNQNARLARM |
| | | | | | GLGFSTASQLVFGIERIAVIWIGAT |
| | | | | | MTLDNVFSVGMLVAYLAYKDQFAKR |
| | | | | | VSALIDKWIEFRMLRLHGERLADIV |
| | | | | | LTPPECDYALPEVLPPTEPRIEVEG |
| | | | | | LSFRYAEGEPWVLKECSLSIHAGES |
| | | | | | VAIIGPSGCGKTTLLKLLLGLLQPT |
| | | | | | EGVIRIGGHDVHKIGPRNVRAIVGA |
| | | | | | VMQDDQLFAGSIADNISFFDQDFDL |
| | | | | | ERIEAAARLAAVHEDIAAMPMGYHG |
| | | | | | LIGDMGSSLSGGQKQRIILARALYR |
| | | | | | QPKLLFLDEATSHLDVMCERLVNEA |
| | | | | | VKQLKVTKVLVAHRPETIASADRVI |
| | | | | | VLEQGRVVQEFHPHAALSDVLPARD |
| | | | | | IERAHGPAVV |
| 2 | WP_099802567.1 | MULTISPECIES: | Xanthomonas | 100 | MTMADGSPTLLWSQKKRVDPIMQSE |
| | | peptidase | | | AAECALACLAMLAQFYGNHCGLPAL |
| | | domain- | | | RRRYSSTLKGMSLNQVIEVADDMGF |
| | | containing | | | DCRALRAELDYLREVQAPVILHWNM |
| | | ABC | | | NHFVVLAGTKGSKLVIIDPAQGRRL |
| | | transporter | | | MPMGEVSKYFTGVLLELEPSASFRR |
| | | | | | ASKSPTVPLSALTGRISGLKRVLIQ |
| | | | | | IFALALAIEALGLSIPLQAQWAVDQ |
| | | | | | ISESNGKVILTVAAIFSLVIIIQAG |
| | | | | | LNLARAWLISWLGANISTQWVINIF |
| | | | | | SHLMRLPLDFFEKRHLGDIMSRFGS |
| | | | | | IHWIQAILTGSFVTALLDGLTGSLA |
| | | | | | LALLFIYSTGIATIAVVVATLYALG |
| | | | | | RFLMFGALWRASEQSMVLDARQQSE |
| | | | | | LMESVRGIQAIKLANRQLERRARLA |
| | | | | | NATLEAAKRGVTAERIKLGFGAASQ |
| | | | | | GIFGLQRVFLLSAGAYLISRNAMTA |
| | | | | | GMMVAALSYADQFSTRVGSLIDNAV |
| | | | | | ELKMLGLHLNRLADITTADEEPARG |
| | | | | | RLEGALRSAPEVQMKGLGYRYSEWE |
| | | | | | PWIFRNLSFTIAPGQSVALVGPSGC |
| | | | | | GKTTLAKIVLGLLAPQEGEVTVTDQ |
| | | | | | PRPVCRSAIPAEGMAAVMQDDCLFS |
| | | | | | GTIADNVAFFDSSAELSEIEAAARA |
| | | | | | AGIHDEILKMPMGYETLVGDMGSTL |
| | | | | | SGGQKQRLLLARALYRQPKILVLDE |
| | | | | | ATSHLDSDNEKVVNDAVMDLEITRI |
| | | | | | IIAHRKETIAMADRVFDLSTQTWIR |
| 2 | WP_099803262.1 | peptidase | Xanthomonas | 101 | MTMADGSPTLLWSQKKRVDPIMQSE |
| | | domain- | citri | | AAECALACLAMLAQFYGNHCGLPAL |
| | | containing | | | RRRYSSTLKGMSLNQVIEVADDMGF |
| | | ABC | | | DCRALRAELDYLREVQAPVILHWNM |
| | | transporter | | | NHFVVLAGTKGSKLVIIDPAQGRRL |
| | | | | | MPMGEVSKYFTGVLLELEPSASFRR |
| | | | | | ASKSPAVPLSALTGRISGLKRVLIQ |
| | | | | | IFALALAIEALGLSIPLQAQWAVDQ |
| | | | | | ISESNGKVILTVAAIFSLVIIIQAG |
| | | | | | LNLARAWLISWLGANISTQWVINIF |
| | | | | | SHLMRLPLDFFEKRHLGDIMSRFGS |
| | | | | | IHWIQAILTGSFVTALLDGLTGSLA |
| | | | | | LALLCIYSTGMATVAVVVATLYALG |
| | | | | | RFLMFGALWRASEQSMVLDARQQSE |
| | | | | | LMESVRGIQAIKLANRQLERRARLA |
| | | | | | NATLEAAKRGVTAERIKLGFGAASQ |
| | | | | | GIFGLQRVFLLSAGAYLISRNAMTA |
| | | | | | GMMVAALSYADQFSTRVGSLIDNAV |
| | | | | | ELKMLGLHLNRLADITTADEEPARG |
| | | | | | RLEGALRSAPEVQMKGLGYRYSEWE |
| | | | | | PWIFRNLSFTIAPGQSVALVGPSGC |
| | | | | | GKTTLAKIVLGLLAPQEGEVTVTDQ |
| | | | | | PRPVCRSAIPAEGMAAVMQDDCLFS |
| | | | | | GTIADNVAFFDSSAELSEIEAAARA |
| | | | | | AGIHDEILKMPMSYETLVGDMGSTL |
| | | | | | SGGQKQRILLARALYRQPKILVLDE |
| | | | | | ATSHLDSDNEKVVNDAVMDLEITRI |
| | | | | | IIAHRKETIAMADRVFDLSTQTWIR |
| 2 | WP_099803931.1 | peptidase | Xanthomonas | 102 | MKDVLTPSANGDAETDRSAEPSLVF |
| | | domain- | citri | | SSKRRVPHIRQTESSECGLACIAML |
| | | containing | | | LSYYGHETSLGELRNRFTVSTSGAT |
| | | ABC | | | LASLIEIADANGLTTRPLRLELYEL |
| | | transporter | | | PKLSLPCMVHLHHGHFVVLTAVRGG |
| | | | | | HVHISDPATGVKKIKLDEFDELFSG |
| | | | | | IALEAHPGPAFKKIKSVPSVSLRDL |
| | | | | | AGSLRSLVPAFWVVGILALFLEGCA |
| | | | | | LIAPQYLRLTMDQVLSVKDDGLVTT |
| | | | | | LAIGFSIVLLVQLVLTVARKWTLLW |
| | | | | | ISSTTGLRWSSNLFRHLVSLPQSYF |
| | | | | | VKRHTGDISSRFQSIYSIQQSLTTK |
| | | | | | MLEAVIDGVMSFLMLLLLMSYDAPL |
| | | | | | SFALLAFTIAYVGSRYAYYAKLKEA |
| | | | | | NLDQINIDARRQSLLYETVRSIQPI |
| | | | | | KLFNKSALWASRFTNYSAKATNATI |
| | | | | | GAERIRIGFDSVQLLIQGLCRIFVI |
| | | | | | WEGTRLVLSGDMTIGVLTVFLIYAT |
| | | | | | QFSQRSINLADYLMQLRLLRLHTER |
| | | | | | ISDITHSEPESFLHGNGALEDAAPA |
| | | | | | IAIANGYFRYSSTDKWIMSALQLRA |
| | | | | | PAGQVIAIVGNSGVGKTTLIRVLAG |
| | | | | | LEDLQVGDFLVNGEDLRKVGKSSYR |
| | | | | | SKVSIVMQGDNLLSGTLAENISMFD |
| | | | | | EHIDQERLSQAAQLACIHDDIQRMP |
| | | | | | MGFNTRVGDLGNTLSGGQKQRIFLA |
| | | | | | RAFYRRTKLLLMDEPTTGVDERMGI |
| | | | | | QLMKNIKSIGATTVIVTHDKNISRM |
| | | | | | CDMHYLFVGGNLKPLIKEKSEQSEA |
| | | | | | NEFAEQLKIQARNEE |
| 2 | WP_011258532.1 | peptidase | Xanthomonas | 103 | MKAIVQAEASECGLASLAMVASAHG |
| | | domain- | oryzae | | MQLDLPELRRRFHLSLKGIRLNQLI |
| | | containing | | | EIAQTLGFSTRPLRLEMEQLDQLSL |
| | | ABC | | | PCILHWDLNHFVVLAKVGKSKATIL |
| | | transporter | | | DPAIGERRLSLGEVSQHFTGVALEL |
| | | | | | TPTAEFKQHKAAPSISARQLTGPIR |
| | | | | | GLWSALSQIALLSLALQVFVILAPF |
| | | | | | YTQWVVDQVLVSADRDLLVVLGLGF |
| | | | | | GLALLLQVGIGLLRGWSVVSLSSRL |
| | | | | | GLQWMGNVFAHLLKLPLDFFEKRHL |
| | | | | | GDVTSRMSSVQTIQHTLTTSFVEAM |
| | | | | | IDGVMAMVTLVLMLVYSWKLALVTL |
| | | | | | LAVALYLGIRAIAYRPMRDRTEQQL |
| | | | | | VAAAKQQTHLLESLRGMQSLKVAGE |
| | | | | | ESVRRSTYENLLNDTVNQDVKLARM |
| | | | | | SLGENTASQLVFGLERIAVIWIGAR |
| | | | | | LALDNVFSVGMLVAYLAYKDQFAMR |
| | | | | | VSGLIDKWIEFRMLRLHGERLADIV |
| | | | | | LTPPEKQHAQPHALPPAEPSIEVEG |
| | | | | | LSFRYADGEPWVVKDCSFTIAPGES |
| | | | | | VAIIGGSGCGKTTLVKLLLGLLTPS |
| | | | | | EGTIRIGGHDLHKLGPRTVRAMIGV |
| | | | | | VMQDDQLFAGSIADNIGFFDTDFDL |
| | | | | | ERIKAAAQLAAVHEDIAAMPMGYHS |
| | | | | | LIGDMGSSLSGGQKQRIILARALYR |
| | | | | | QPKLLFLDEATSHLDVTRERLVNEA |
| | | | | | VKHLQLTKVIVAHRPETIASADRIL |
| | | | | | VMEHGRIVQEVKLQQSPEIPHPEDH |
| | | | | | VLSA |
| 2 | WP_011408204.1 | peptidase | Xanthomonas | 104 | MKAIVQAEASECGLASLAMVASAHG |
| | | domain- | oryzae | | MQLDLPELRRRFHLSLKGIRLNQLI |
| | | containing | | | EIAQTLGFSTRPLRLEMEQLDQLSL |
| | | ABC | | | PCILHWDLNHFVVLAKVGKSKATIL |
| | | transporter | | | DPAIGERRLSLGEVSQHFTGVALEL |
| | | | | | TPTAEFKQHKAAPSISARQLTGPIR |
| | | | | | GLWSALSQIALLSLALQVFVILAPF |
| | | | | | YTQWVVDQVLVSADRDLLVVLGLGF |
| | | | | | GLALLLQVGIGLLRGWSVVSLSSRL |
| | | | | | GLQWMGNVFAHLLKLPLDFFEKRHL |
| | | | | | GDVTSRMSSVQTIQHTLTTSFVEAM |
| | | | | | IDGVMAMVTLVLMLVYSWKLALVTL |
| | | | | | LAVALYLGIRAIAYRPMRDRTEQQL |
| | | | | | VAAAKQQTHLLESLRGMQSLKVAGE |
| | | | | | ESVRRSTYENLLNDTVNQDVKLARM |
| | | | | | SLGFNTASQLVFGLERIAVIWIGAR |
| | | | | | LALDNVFSVGMLVAYLAYKDQFAMR |
| | | | | | VSGLIDKWIEFRMLRLHGERLADIV |
| | | | | | LTPPEKQHAQPHALPPAEPSIEVEG |
| | | | | | LSFRYADGEPWVVKDCSFTIAPGES |
| | | | | | VAIIGGSGCGKTTLVKLLLGLLTPS |
| | | | | | EGTIRIGGHDLHKLGPRNVRAMIGV |
| | | | | | VMQDDQLFAGSIADNIGFFDTDFDL |
| | | | | | ERIKAAAQLAAVHEDIAAMPMGYHS |
| | | | | | LIGDMGSSLSGGQKQRIILARALYR |
| | | | | | QPKLLFLDEATSHLDVTRERLVNEA |
| | | | | | VKHLQLTKVIVAHRPETIASADRIL |
| | | | | | VMEHGRIVQEVKLQQSPEIPRPEDH |
| | | | | | VLSA |
| 2 | WP_011409245.1 | peptidase | Xanthomonas | 105 | MSRWLAQLRRASGERALPMILQGQV |
| | | domain- | oryzae | | GECGLAAMAMIAHYHGCQIGLAELR |
| | | containing | | | RRFLLSRQGTNLANLVAIAQALGLQ |
| | | ABC | | | ARALRLEMDGVPDLQLPCIVHWDLN |
| | | transporter | | | HFVVLKRAGARRLQIHDPASGPRVL |
| | | | | | TPGEFARHFSGIALELSPTADFRAQ |
| | | | | | AAAPPVALSSLIGRVHGLGRAVWQV |
| | | | | | LALAFALEVLSLGMPFQLQWIVDQA |
| | | | | | VPSADIGLIHVLGAGFLLVVVLQSC |
| | | | | | IGLLRGWLIASVSAQLGFQWMGQVF |
| | | | | | AHLLALPLAYFEKRHLGGIQSRFAS |
| | | | | | ITQVQRTLTTGFTQTLVDGVLVVGT |
| | | | | | LGLMLVYSAGLSAITLVAVALYAAT |
| | | | | | RLLWLSRMREATAEQLLWDARQHTH |
| | | | | | LLESVRCIQGVRLFGRQQVRRMDWT |
| | | | | | HLLAEQTNAQLRLAQGEVWQSAIKL |
| | | | | | LLFGGERVLVIWLAAFAILRAELSL |
| | | | | | GMLLAFMAYREQFAMRLSALIDRLV |
| | | | | | EFRLLRVHLERVADIVHQPREDADQ |
| | | | | | RVDAPDWSDTTIEVCGIGFRYADDA |
| | | | | | PAVLEEVNIRIGSGECVAITGPSGC |
| | | | | | GKTTLVKVILGLLQPSTGQVKIGGR |
| | | | | | PLTRAALAHYRGIVGTVMQDDLLFT |
| | | | | | GSVSENISFFDPEPDQGQIERCARI |
| | | | | | AGVHQEVEQMPLGYASLLSEAGTGL |
| | | | | | SGGQRQRVLLARALYRAPRILVLDE |
| | | | | | ATSHLDVINEHRVNHAIQAMQVTRI |
| | | | | | IVAHRRETVSMAARVITLERGRVTS |
| | | | | | DQPIAAWQRLQEHSAAAAD |
| 2 | WP_012444311.1 | peptidase | Xanthomonas | 106 | MSRWLAQLRRASGARALPMILQGQV |
| | | domain- | oryzae | | GECGLAAMAMIAHYHGCQIGLAELR |
| | | containing | | | RRFLLSRQGTNLANLVAIAQALGLQ |
| | | ABC | | | ARALRLEMDGVPDLQLPCIVHWDLN |
| | | transporter | | | HFVVLKRVGTRRLQIHDPASGPRGL |
| | | | | | TPGEFARHFSGIALELSPTADFRAQ |
| | | | | | SAAPPVALSSLIGRVHGLGRAVWQV |
| | | | | | LALAFALEVLSLGMPFQLQWIVDQA |
| | | | | | VPSADIGLIHVLGAGFLLLVVLQSC |
| | | | | | IGLLRGWLIASVSAQLGFQWMGQVF |
| | | | | | AHLLALPLAYFEKRHLGGIQSRFAS |
| | | | | | ITQVQRTLTTGFTQTLVDGVLVVGT |
| | | | | | LGLMLVYSAGLSAITLVAVALYAAT |
| | | | | | RLLWLSRMREATAEQLLWDGRQHTH |
| | | | | | LLESVRCIQGVRLFGRQQVRRMDWT |
| | | | | | HLLAEQTNAQLRLAQGEVWQSAIKL |
| | | | | | LLFGGERVLVIWLAAFAILRAELSL |
| | | | | | GMLLAFMAYREQFAMRLSALIDRLV |
| | | | | | EFRLLRVHLERVADIVHQPREDADQ |
| | | | | | RVDAPDWSDTTIEVCGIGFRYADDA |
| | | | | | PAVLEEVNIRIGSGECVAITGPSGC |
| | | | | | GKTTLVKVILGLLQPSTGQVKIGGR |
| | | | | | PLTRAALAHYRGIVGTVMQDDLLFT |
| | | | | | GSVSENISFFDPEPDQGQIERCARI |
| | | | | | AGVHQEVEQMPLGYASLLSEAGTGL |
| | | | | | SGGQRQRVLLARALYRAPRILVLDE |
| | | | | | ATSHLDVINEHRVNHAIQAMQVTRI |
| | | | | | IVAHRRETVSMAARVITLERGRVTS |
| | | | | | DQPIAAWQRLQEHSAAAAD |
| 2 | WP_012445305.1 | peptidase | Xanthomonas | 107 | MKPILQAEASECGLASLAMVASAHG |
| | | domain- | oryzae | | MQLDLPELRRQFHLSLKGIRLNQLI |
| | | containing | | | EIAQTLGFSTRPLRLEMEQLDQLSL |
| | | ABC | | | PCILHWDLNHFVVLAKVGKSKATIL |
| | | transporter | | | DPAIGERRLSLGEVSQHFTGVALEL |
| | | | | | TPTAEFKQHKAAPSISARQLTGPIR |
| | | | | | GLWSALSQIALLSLALQVFVILAPF |
| | | | | | YTQWVVDQVLVSADRDLLVVLGLGF |
| | | | | | GLALLLQVGIGLLRGWSVVSLSSRL |
| | | | | | GLQWMGNVFAHLLKLPLDFFEKRHL |
| | | | | | GDVTSRMSSVQTIQHTLTTSFVEAM |
| | | | | | IDGVMAMVTLVLMLVYSWKLALVTL |
| | | | | | LAVALYLGIRAIAYRPMRDRTEQQL |
| | | | | | VAAAKQQTHLLESLRGMQSLKVAGE |
| | | | | | ESVRRSTYENLLNDTVNQDVKLARM |
| | | | | | SLGFNTASQLVFGLERIAVIWIGAR |
| | | | | | LALDNVFSVGMLVAYLAYKDQFAMR |
| | | | | | VSGLIDKWIEFRMLRLHGERLADIV |
| | | | | | LTPPEKQHAQPHALPPAEPSIEVEG |
| | | | | | LSFRYADGEPWVVKDCSFTIAPGES |
| | | | | | VAIIGGSGCGKTTLVKLLLGLLTPS |
| | | | | | EGTIRIGGHDLHKLGPRNVRAMIGV |
| | | | | | VMQDDQLFAGSIADNIGFFDTDFDL |
| | | | | | ERIKAAAQLAAVHEDIAAMPMGYHS |
| | | | | | LIGDMGSSLSGGQKQRIILARALYR |
| | | | | | QPKLLFLDEATSHLDVTRERLVNEA |
| | | | | | VKHLQLTKVIVAHRPETIASADRIL |
| | | | | | VMEHGRIVQEVKLQQSPEIPRPEDH |
| | | | | | VLSA |
| 2 | WP_014501285.1 | peptidase | Xanthomonas | 108 | MTMADGSPTLLWSQKNRVDPIMQSE |
| | | domain- | oryzae | | AAECALACLAMLAQFYGNDCGLPAL |
| | | containing | | | RRRYSSTLKGMSLNQVIEVADDMGF |
| | | ABC | | | DCRALRAELDYLREVQAPVILHWNM |
| | | transporter | | | NHFVVLAGTKGSKLVIIDPAQGRRL |
| | | | | | MPMGEVSKYFTGVMLELEPSASFRR |
| | | | | | ASKSPTVPLSALTGRISGLKRVLIQ |
| | | | | | IFALALAIEALGLSIPLQAQWTVDQ |
| | | | | | ISESNGKVILTVAAIFSLVIIIQVG |
| | | | | | LNLARAWLISWLGANISTQWVINIF |
| | | | | | SHLMRLPLDFFEKRHLGDIMSRFGS |
| | | | | | IHWIQSILTGSFVTALLDGLTGSLA |
| | | | | | LVLLFIYSTGMATVAVAVATLYALG |
| | | | | | RFLMFGALWRASEQSMVFDARQQSE |
| | | | | | LMESVRGIQAIKLANRQLERRARLA |
| | | | | | NATLEAAKRGVTAERIKLGFGAASQ |
| | | | | | GIFGLQRVFLLSAGAYLISRNAMTA |
| | | | | | GMMVAALSYADQFSTRVGSLIDNAV |
| | | | | | ELKMLGLHLNRLADITTADQEPARG |
| | | | | | RLEGALRSAPEVQVKGLGYRYSEWE |
| | | | | | PWIFRNLSFTIAPGQSVALVGPSGC |
| | | | | | GKTTLAKIVLGLLAPQEGEVTVTDQ |
| | | | | | PSPVCRSAIPAKGMAAVMQDDCLFS |
| | | | | | GTIADNVAFFDSSAELSEIEAAARA |
| | | | | | AGIYDDIVKMPMGYETLVGDMGSTL |
| | | | | | SGGQKQRILLARALYTQPKILVLDE |
| | | | | | ATSHLDSKNEKFVNDAVMDLKITRI |
| | | | | | IIAHREQTIAMADRVFDLSRQTWIR |
| 2 | WP_027703411.1 | peptidase | Xanthomonas | 109 | MKPILQAEASECGLASLAMVASAHG |
| | | domain- | oryzae | | MQLDLPELRRRFHLSLKGIRLNQLI |
| | | containing | | | EIAQTLGCSTRPLRLEMEQLDQLSL |
| | | ABC | | | PCILHWDLNHFVVLAKVGKSKATIL |
| | | transporter | | | DPVIGERRLSLGEVSQHFTGVALEL |
| | | | | | TPTAEFKQHKAAPSISARQLTGPIR |
| | | | | | GLWSALSQIALLSLALQVFVILAPF |
| | | | | | YTQWVVDQVLVSADRDLLVVLGLGF |
| | | | | | GLALLLQVGIGLLRGWSVVSLSSRL |
| | | | | | GLQWMGNVFAHLLKLPLDFFEKRHL |
| | | | | | GDVTSRMSSVQTIQHTLTTSFVEAM |
| | | | | | IDGVMAMVTLVLMLVYSWKLALVTL |
| | | | | | LAVALYLGIRAIAYRPMRDRTEQQL |
| | | | | | VAAAKQQTHLLESLRGMQSLKVAGE |
| | | | | | ESVRRSTYENLLNDTVNQDVKLARM |
| | | | | | SLGFNTASQLVFGLERIAVIWIGAR |
| | | | | | LALDNVFSVGMLVAYLAYKDQFAMR |
| | | | | | VSGLIDKWIEFRMLRLHGERLADIV |
| | | | | | LTPPEKQHAQPHALPPAEPSIEVEG |
| | | | | | LSFRYADGEPWVVKDCSFTIAPGES |
| | | | | | VAIIGGSGCGKTTLVKLLLGLLTPS |
| | | | | | EGTIRIGGHDLHKLGPRNVRAMIGV |
| | | | | | VMQDDQLFAGSIADNIGFFDTDFDL |
| | | | | | ERIKAAAQLAAVHEDIAAMPMGYHS |
| | | | | | LIGDMGSSLSGGQKQRIILARALYR |
| | | | | | QPKLLFLDEATSHLDVTRERLVNEA |
| | | | | | VKHLQLTKVIVAHRPETIASADRIL |
| | | | | | VMEHGRIVQEVKLQQSPEIPRPEDH |
| | | | | | VLSA |
| 2 | WP_041182251.1 | peptidase | Xanthomonas | 110 | MSRWLAQLRRASGERALPMILQGQV |
| | | domain- | oryzae | | GECGLAAMAMIAHYHGCQIGLAELR |
| | | containing | | | RRFLLSRQGTNLANLVAIAQALGLQ |
| | | ABC | | | ARALRLEMDGVPDLQLPCIVHWDLN |
| | | transporter | | | HFVVLKRAGARRLQIHDPASGPRVL |
| | | | | | TPGEFARHFSGIALELSPTADFRAQ |
| | | | | | AAAPPVALSSLIGRVHGLGRAVWQV |
| | | | | | LALAFALEVLSLGMPFQLQWIVDQA |
| | | | | | VPSADIGLIHVLGAWFLLVVVLQSC |
| | | | | | IGLLRGWLIASVSAQLGFQWMGQVF |
| | | | | | AHLLALPLAYFEKRHLGGIQSRFAS |
| | | | | | ITQVQRTLTTGFTQTLVDGVLVVGT |
| | | | | | LGLMLVYSAGLSAITLVAVALYAAT |
| | | | | | RLLWLSRMREATAEQLLWDARQHTH |
| | | | | | LLESVRCIQGVRLFGRQQVRRMDWT |
| | | | | | HLLAEQTNAQLRLAQGEVWQSAIKL |
| | | | | | LLFGGERVLVIWLAAFAILRAELSL |
| | | | | | GMLLAFMAYREQFAMRLSALIDRLV |
| | | | | | EFRLLRVHLERVADIVHQPREDADQ |
| | | | | | RVDAPDWSDTTIEVCGIGFRYADDA |
| | | | | | PAVLEEVNIRIGSGECVAITGPSGC |
| | | | | | GKTTLVKVILGLLQPSTGQVKIGGR |
| | | | | | PLTRAALAHYRGIVGTVMQDDLLFT |
| | | | | | GSVSENISFFDPEPDQGQIERCARI |
| | | | | | AGVHQEVEQMPLGYASLLSEAGTGL |
| | | | | | SGGQRQRVLLARALYRAPRILVLDE |
| | | | | | ATSHLDVINEHRVNHAIQAMQVTRI |
| | | | | | IVAHRRETVSMAARVITLERGRVTS |
| | | | | | DQPIAAWQRLQEHSAAAAD |
| 2 | WP_044750783.1 | peptidase | Xanthomonas | 111 | MSRWLAQLRRASGARALPMILQGQV |
| | | domain- | oryzae | | GECGLAAMAMIAHYHGCQIGLAELR |
| | | containing | | | RRFLLSRQGTNLANLVAIAQALGLQ |
| | | ABC | | | ARALRLEMDGVPDLQLPCIVHWDLN |
| | | transporter | | | HFVVLKRVGTRRLQIHDPASGPRGL |
| | | | | | TPSEFARHFSGIALELSPTADFRAQ |
| | | | | | SAAPPVALSSLIGRVHGLGRAVWQV |
| | | | | | LALAFALEVLSLGMPFQLQWIVDQA |
| | | | | | VPSADIGLIHVLGAGFLLLVVLQSC |
| | | | | | IGLLRGWLIASVSAQLGFQWMGQVF |
| | | | | | AHLLALPLAYFEKRHLGGIQSRFAS |
| | | | | | ITQVQRTLTTGFTQTLVDGVLVVGT |
| | | | | | LGLMLVYSAGLSAITLVAVALYAAT |
| | | | | | RLLWLSRMREATAEQLLWDARQHTH |
| | | | | | LLESVRCIQGVRLFGRQQVRRMDWT |
| | | | | | HLLAEQTNAQLRLAQGELWQSAIKL |
| | | | | | LLFGGERVLVIWLAAFAILRAELSL |
| | | | | | GMLLAFMAYREQFAMRLSALIDRLV |
| | | | | | EFRLLRVHLERVADIVHQPREDADQ |
| | | | | | RVDAPDWSDTTIELCGIGFRYADDT |
| | | | | | PAVLEEVNIRIGSGECVAITGPSGC |
| | | | | | GKTTLVKVILGLLQPSTGQVKISGR |
| | | | | | PLTRAALAHYRGIVGTVMQDDLLFT |
| | | | | | GSVSENISFFDPEPDQAQIERCARI |
| | | | | | AGVHQEVEQMPLGYASLLSEAGTGL |
| | | | | | SGGQRQRVLLARALYRAPRILVLDE |
| | | | | | ATSHLDVINEHRVNHAIQAMQMTRI |
| | | | | | IVAHRRETVSMAARVITLERGRVAS |
| | | | | | DQPIAAWQRLQEHSAAAAD |
| 2 | WP_053502191.1 | peptidase | Xanthomonas | 112 | MSRWLAQLRRASGARALPMILQGQV |
| | | domain- | oryzae | | GECGLAAMAMIAHYHGCQIGLAELR |
| | | containing | | | RRFLLSRQGTNLANLVAIAQALGLQ |
| | | ABC | | | ARALRLEMDGVPDLQLPCIVHWDLN |
| | | transporter | | | HFVVLKRVGARRLQIHDPASGPRGL |
| | | | | | TPGEFARHFSGIALELSPTADFRAQ |
| | | | | | SAAPPVALSSLIGRVHGLGRAVWQV |
| | | | | | LALAFALEVLSLGMPFQLQWIVDQA |
| | | | | | VPSADIGLIHVLGAGFLLLVVLQSC |
| | | | | | IGLLRGWLIASVSAQLGFQWMGQVF |
| | | | | | AHLLALPLAYFEKRHLGGIQSRFAS |
| | | | | | ITQVQRTLTTGFTQTLVDGVLVVGT |
| | | | | | LGLMLVYSAGLSAITLVAVALYAAT |
| | | | | | RLLWLSRMREATAEQLLWDARQHTH |
| | | | | | LLESVRCIQGVRLFGRQQVRRMDWT |
| | | | | | HLLAEQTNAQLRLAQGEVWQSAIKL |
| | | | | | LLFGGERVLVIWLAAFAILRAELSL |
| | | | | | GMLLAFMAYREQFAMRLSALIDRLV |
| | | | | | EFRLLRVHLERVADIVHQPREDADQ |
| | | | | | RVDAPDWSDTTIELCGIGFRYADDT |
| | | | | | PAVLEEVNIRIGSGECVAITGPSGC |
| | | | | | GKTTLVKVILGLLQPSTGQVKIGGR |
| | | | | | PLTRAALAHYRGIVGTVMQDDLLFT |
| | | | | | GSVSENISFFDPEPDQAQIERCARI |
| | | | | | AGVHQEVEQMPLGYASLLSEAGTGL |
| | | | | | SGGQRQRVLLARALYRAPRILVLDE |
| | | | | | ATSHLDVINEHRVNHAIQAMQVTRI |
| | | | | | IVAHRRETVSMAARVITLERGRVAS |
| | | | | | DQPIAAWQRLQEHSAAAAD |
| 2 | WP_053503740.1 | peptidase | Xanthomonas | 113 | MKAIVQAEASECGLASLAMVASAHG |
| | | domain- | oryzae | | MQLGLPELRRRFHLSLKGIRLNQLI |
| | | containing | | | EIAQTLGFSTRPLRLDMEQLDQLAL |
| | | ABC | | | PCILHWDLSHFVVLAKVGKSKATIF |
| | | transporter | | | DPAIGERRLSLGEVSQHFTGVALEL |
| | | | | | TPTAEFKQHKAAPSISARQLTGPIR |
| | | | | | GLWSALSQIALLSMALQVFVILAPF |
| | | | | | YTQWVVDQVLVSADRDLLVVLGLGF |
| | | | | | GLALLLQVGIGLLRGWSVVSLSSRL |
| | | | | | GLQWMGNVFAHLLKLPLDFFEKRHL |
| | | | | | GDVTSRMSSVQTIQHTLTTSFVEAM |
| | | | | | IDGVMAMVTLVLMLVYSWKLALVTL |
| | | | | | LAVALYLGIRAIAYRPMRDRTEQQL |
| | | | | | VAAAKQQTHLLESLRGMQSLKVAGE |
| | | | | | ESVRRSTYENLLNDTVNQDVKLARM |
| | | | | | SLGFNTASQLVFGLERIAVIWIGAR |
| | | | | | LALDNLFSVGMLVAYLAYKDQFAMR |
| | | | | | VSGLIDKWIEFRMLRLHGERLADIV |
| | | | | | LTPPEEHHAQPRALPPTEPSIEVEG |
| | | | | | LSFRYADGEPWVVKDCGFTIAPGES |
| | | | | | VAIIGGSGCGKTTLVKLLLGLLVPS |
| | | | | | EGTIRIGGHDLHKLGPRTVRAMIGV |
| | | | | | VMQDDQLFAGSIADNIGFFDTDFEL |
| | | | | | ERIEAAAQLAAVHEDIAAMPMGYHT |
| | | | | | LIGDMGSSLSGGQRQRIILARALYR |
| | | | | | QPKLLFLDEATSHLDVTRERLVNEA |
| | | | | | VRHLQLTKVIVAHRPETIASADRIL |
| | | | | | VMAHGRIVQEVKSQQSPEISCPEDH |
| | | | | | VLSA |
| 2 | WP_075239139.1 | peptidase | Xanthomonas | 114 | MKPILQAEASECGLASLAMVASAHG |
| | | domain- | oryzae | | MQLDLPELRRRFHLSLKGIRLNQLI |
| | | containing | | | EIAQTLGCSTRPLRLEMEQLDQLSL |
| | | ABC | | | PCILHWDLNHFVVLAKVGKSKATIL |
| | | transporter | | | DPAIGERRLSLGEVSQHFTGVALEL |
| | | | | | TPTAEFKQHKAAPSISARQLTGPIR |
| | | | | | GLWSALSQIALLSLALQVFVILAPF |
| | | | | | YTQWVVDQVLVSADRDLLVVLGLGF |
| | | | | | GLALLLQVGIGLLRGWSVVSLSSRL |
| | | | | | GLQWMGNVFAHLLKLPLDFFEKRHL |
| | | | | | GDVTSRMSSVQTIQHTLTTSFVEAM |
| | | | | | IDGVMAMVTLVLMLVYSWKLALVTL |
| | | | | | LAVALYLGIRAIAYRPMRDRTEQQL |
| | | | | | VAAAKQQTHLLESLRGMQSLKVAGE |
| | | | | | ESVRRSTYENLLNDTVNQDVKLARM |
| | | | | | SLGFNTASQLVFGLERIAVIWIGAR |
| | | | | | LALDNVFSVGMLVAYLAYKDQFAMR |
| | | | | | VS |
| | | | | | GLIDKWIEFRMLRLHGERLADIVLT |
| | | | | | PPEKQHAQPHALPPAEPSIEVEGLS |
| | | | | | FRYADGEPWVVKDCSFTIAPGESVA |
| | | | | | IIGGSGCGKTTLVKLLLGLLTPSEG |
| | | | | | TIRIGGHDLHKLGPRNVRAMIGVVM |
| | | | | | QDDQLFAGSIADNIGFFDTDFDLER |
| | | | | | IKAAAQLAAVHEDIAAMPMGYHSLI |
| | | | | | GDMGSSLSGGQKQRIILARALYRQP |
| | | | | | KLLFLDEATSHLDVTRERLVNEAVK |
| | | | | | HLQLTKVIVAHRPETIASADRILVM |
| | | | | | EHGRIVQEVKLQQSPEIPRPEDHVL |
| | | | | | SA |
| 2 | WP_075240503.1 | peptidase | Xanthomonas | 115 | MSRWLAQLRRASGARALPMILQGQV |
| | | domain- | oryzae | | GECGLAAMAMIAHYHGCQIGLAELR |
| | | containing | | | RRFLLSRQGTNLANLVAIAQALGLQ |
| | | ABC | | | ARALRLEMDGVPDLQLPCIVHWDLN |
| | | transporter | | | HFVVLKRAGARRLQIHDPASGPRVL |
| | | | | | TPGEFARHFSGIALELSPTADFRAQ |
| | | | | | AAAPPVALSSLIGRVHGLGRAVWQV |
| | | | | | LALAFALEVLSLGMPFQLQWIVDQA |
| | | | | | VPSADIGLIHVLGAGFLLVVVLQSC |
| | | | | | IGLLRGWLIASVSAQLGFQWMGQVF |
| | | | | | AHLLALPLAYFEKRHLGGIQSRFAS |
| | | | | | ITQVQRTLTTGFTQTLVDGVLVVGT |
| | | | | | LGLMLVYSAGLSAITLVAVALYAAT |
| | | | | | RLLWLSRMREATAEQLLWDARQHTH |
| | | | | | LLESVRCIQGVRLFGRQQVRRMDWT |
| | | | | | HLLAEQTNAQLRLAQGEVWQSAIKL |
| | | | | | LLFGGERVLVIWLAAFAILRAELSL |
| | | | | | GMLLAFMAYREQFAMRLSALIDRLV |
| | | | | | EFRLLRVHLERVADIVHQPREDADQ |
| | | | | | RVDAPDWSDTTIEVCGIGFRYADDA |
| | | | | | PAVLEEVNIRIGSGECVAITGPSGC |
| | | | | | GKTTLVKVILGLLQPSTGQVKIGGR |
| | | | | | PLTRAALAHYRGIVGTVMQDDLLFT |
| | | | | | GSVSENISFFDPEPDQGQIERCARI |
| | | | | | AGVHQEVEQMPLGYASLLSEAGTGL |
| | | | | | SGGQRQRVLLARALYRAPRILVLDE |
| | | | | | ATSHLDVINEHRVNHAIQAMQVTRI |
| | | | | | IVAHRRETVSMAARVITLERGRVTS |
| | | | | | DQPIAAWQRLQEHSAAAAD |
| 2 | WP_010381219.1 | peptidase | Xanthomonas | 116 | MKAIVQAEASECGLASLAMVASAHG |
| | | domain- | vasicola | | MQLGLPELRRRFHLSLKGIRLNQLI |
| | | containing | | | EIAQTLGFSTRPLRLEMEQLDQLAL |
| | | ABC | | | PCILHWDLNHFVVLAKVGKTKATIL |
| | | transporter | | | DPAIGDRRLSLSEVSQHFTGVALEL |
| | | | | | TPTTEFKQHKAAPSISARQLTGPIR |
| | | | | | GLWSALSQITLLSLALQVFVILAPF |
| | | | | | YTQWVVDQVLVSADRDLLVVLGLGF |
| | | | | | GLALLLQVGIGLLRGWSVVSLSSRL |
| | | | | | GLQWMGNVFAHLLKLPLDFFEKRHL |
| | | | | | GDVTSRMSSVQTIQHTLTTSFVEAM |
| | | | | | IDGVMAMVTLVLMLVYSWKLALVTL |
| | | | | | LAVALYLGIRAIAYRPMRDRTEQQL |
| | | | | | VAAAKQQTHLLESLRGMQSLKVAGK |
| | | | | | ESVRRSTYENLLNDTVNQDVKLARM |
| | | | | | SLGFNTASQLVFGLERIAVIWIGAR |
| | | | | | LALDNVFSVGMLVAYLAYKDQFAMR |
| | | | | | VSGLIDKWIEFRMLRLHGERLADIV |
| | | | | | LTPPEEHHAQPHALPPAEPSIAVEG |
| | | | | | LSFRYADGEPWVVKDCCFTIAPGES |
| | | | | | VAIIGGSGCGKTTLVKLLLGLLTPS |
| | | | | | EGTIRIGGHDLHKLGPRNVRAMIGV |
| | | | | | VMQDDQLFAGSIADNIGFFDTDFDL |
| | | | | | ERIEAAAQLAAVHEDIAAMPMGYHS |
| | | | | | LIGDMGSSLSGGQKQRIILARALYR |
| | | | | | QPKLLFLDEATSHLDVTRERLVNEA |
| | | | | | VKHLQLTKVIVAHRPETIASADRIL |
| | | | | | VMEHGRIVQEVKLQQSPEIPHPEDH |
| | | | | | VLSA |
| 2 | WP_017112712.1 | peptidase | Xanthomonas | 117 | MSRWWAQLRRASGLRALPMILQGQV |
| | | domain- | vasicola | | GECGLAAMAMIAHHHGCHIGLAELR |
| | | containing | | | RRFLLSRQGTNLANLVAIAQALGLQ |
| | | ABC | | | PRALRLEMDALPDLQLPCIVHWDLN |
| | | transporter | | | HFVVLKRVGANRLQIHDPASGPRSL |
| | | | | | TPGEFARHFSGIALELSPTADFRPQ |
| | | | | | SAAPAVALSSLIGRVHGLGRAVWQV |
| | | | | | LALAFALEVLSLGMPFQLQWIVDQA |
| | | | | | VPSADIGLIHVLGAGFLLLVVLQAC |
| | | | | | IGLLRGWLIASVSAQLGFQWMGQVF |
| | | | | | AHLLSLPLAYFEKRHLGGIQSRFAS |
| | | | | | ITQVQRTLTTGFTQTLVDGVLVVGT |
| | | | | | LGLMLVYSVGLSAITLVAVALYAAT |
| | | | | | RLLWLGRMREAAAEQLLWDARQHTH |
| | | | | | LLESVRGIQGVRLFGRQQVRRMDWT |
| | | | | | HLLAEQTNAQLRLAQGEVWQSSIKL |
| | | | | | LLFGCERVLVIWLAAFAILRAQLSL |
| | | | | | GMLLAFMAYREQFATRLSELIDRLV |
| | | | | | EFRLLRVHLERVADIVHQPREEAGQ |
| | | | | | RIDAPAWNDTTIELCGIGFRYADDT |
| | | | | | PAVLEDVSVRIASGECVAITGPSGC |
| | | | | | GKTTLVKLILGLLKPSAGQVKIGGR |
| | | | | | PLADAALAHYRAIVGTVMQDDLLFT |
| | | | | | GSVSENISFFDPEPDQAQIERCARI |
| | | | | | AGVHQEVEQMPLGYASLLSEAGTGL |
| | | | | | SGGQRQRVLLARALYRSPRILVLDE |
| | | | | | ATSHLDVMNEHRVNRAIQAMQVTRI |
| | | | | | IVAHRRETVGMAARVITLERGRVVS |
| | | | | | DQPIAAWQRLQERSAAAD |
| 2 | WP_039437388.1 | peptidase | Xanthomonas | 118 | MSRWWAQLRRASGVRALPMILQGQV |
| | | domain- | vasicola | | GECGLAAMAMIAHHHGCHIGLAELR |
| | | containing | | | RRFLLSRQGTNLANLVAIAQALGLQ |
| | | ABC | | | PRALRLEMDALPDLQLPCIVHWDLN |
| | | transporter | | | HFVVLKRVGAHRLQIHDPASGPRSL |
| | | | | | TPGEFARHFSGIALELSPTADFRPQ |
| | | | | | SAAPAVALSSLIGRVHGLGRAVWQV |
| | | | | | LALAFALEVLSLGMPFQLQWIIDQA |
| | | | | | VPSADIGLIHVLGAGFLLLVVLQAC |
| | | | | | IGLLRGWLIASVSAQLGFQWMGQVF |
| | | | | | AHLLSLPLAYFEKRHLGGIQSRFAS |
| | | | | | ITQVQRTLTTGFTQTLVDGVLVVGT |
| | | | | | LGLMLVYSVGLSAITLVAVALYAAT |
| | | | | | RLLWLGRMREAAAEQLLWDARQHTH |
| | | | | | LLESVRGIQGVRLFGRQQVRRMDWT |
| | | | | | HLLAEQTNAQLRLAQGEVWQSSIKL |
| | | | | | LLFGCERVLVIWLAAFAILRAQLSL |
| | | | | | GMLLAFMAYREQFATRLSELIDRLV |
| | | | | | EFRLLRVHLERVADIVHQPREEAGQ |
| | | | | | RIDAPAWNDTTIELCGIGFRYADDT |
| | | | | | PAVLEDVSVRIASGECVAITGPSGC |
| | | | | | GKTTLVKLILGLLKPSAGQVKIGGR |
| | | | | | PLADAALAHYRAIVGTVMQDDLLFT |
| | | | | | GSVSENISFFDPEPDQAQIERCARI |
| | | | | | AGVHQEVEQMPLGYASLLSEAGTGL |
| | | | | | SGGQRQRVLLARALYRSPRILVLDE |
| | | | | | ATSHLDVMNEHRVNRAIQAMQVTRI |
| | | | | | IVAHRRETVGMAARVITLERGRVVS |
| | | | | | DQPIAAWQRLQERSAAAD |
| 2 | WP_039448278.1 | peptidase | Xanthomonas | 119 | MKAIVQAEASECGLASLAMVASAHG |
| | | domain- | vasicola | | MQLGLPELRRRFHLSLKGIRLNQLV |
| | | containing | | | QIAQTLGFSTRPLRLEMEHLDQLAL |
| | | ABC | | | PCVLHWDLNHFVVLAKVGKTKATIL |
| | | transporter | | | DPAIGERRLSLSEVSQHFTGVALEL |
| | | | | | TPTTEFKQHKAAPSISARQLTGPIR |
| | | | | | GLWSALSQIALLSLALQVFVILAPF |
| | | | | | YTQWVVDQVLVSADRDLLVVLGLGF |
| | | | | | GLALLLQVCIGLLRGWSVVSLSSRL |
| | | | | | GLQWMGNVFAHLLKLPLDFFEKRHL |
| | | | | | GDVTSRMSSVQTIQHTLTTSFVEAM |
| | | | | | IDGVMALVTLVLMLIYSWKLALVTL |
| | | | | | LAVALYLVIRAIAYRPMRDRTEQQL |
| | | | | | VAAAKQQTHLLESLRGMQSLKVAGE |
| | | | | | ESVRRSTYENLLNDTVNQDVKLARM |
| | | | | | SLGFNTASQLVFGLERIAVIWIGAR |
| | | | | | LALDNVFSVGMLVAYLAYKDQFAMR |
| | | | | | VSGLIDKWIEFRMLRLHGERLADIV |
| | | | | | LTPPEEHHAQPHALPPAEPSIAVEG |
| | | | | | LSFRYADGEPWVVKDCCFTIAPGES |
| | | | | | VAIIGGSGCGKTTLVKLLLGLLTPS |
| | | | | | EGTIRIGGHDLHKLGPRNVRAMIGV |
| | | | | | VMQDDQLFAGSIADNIGFFDTDFDL |
| | | | | | ERIEAAAQLAAVHEDIAAMPMGYHS |
| | | | | | LIGDMGSSLSGGQKQRIILARALYR |
| | | | | | QPKLLFLDEATSHLDVTRERLVNEA |
| | | | | | VKHLQLTKVIVAHRPETIASADRIL |
| | | | | | VMEHGRIVQEVKLQQSPEIPHPEDH |
| | | | | | VLSA |
| 2 | WP_126925247.1 | peptidase | Xanthomonas | 120 | MKAIVQAEASECGLASLAMVASAHG |
| | | domain- | vasicola | | MQLGLPELRRRFHLSLKGIRLNQLI |
| | | containing | | | EIAQTLGFSTRPLRLEMEQLDQLAL |
| | | ABC | | | PCFLHWDLNHFVVLAKVGKTKATIL |
| | | transporter | | | DPAIGERRLSLSEVSQHFTGVALEL |
| | | | | | TPTTEFKQHKAAPSISARQLTGPIR |
| | | | | | GLWSALSQIALLSMALQVFVILAPF |
| | | | | | YTQWVVDQVLVSADRDLLVVLGLGF |
| | | | | | GLALLLQVGIGLLRGWSVVSLSSRL |
| | | | | | GLQWMGNVFAHLLKLPLDFFEKRHL |
| | | | | | GDVTSRMSSVQAIQHTLTTSFVEAI |
| | | | | | IDGVMTLVTLVLMLVYSWKLALVIL |
| | | | | | LAVALYLGIRAIAYRPMRDRTEQQL |
| | | | | | VAAAKQQTHLLESLRGMQSLKVAGE |
| | | | | | ESVRRSTYENLLNDTVNQDVKLARM |
| | | | | | SLGFNTASQLVFGLERIAVIWIGAR |
| | | | | | LALDNVFSVGMLVAYLAYKDQFAMR |
| | | | | | VSGLIDKWIEFRMLRLHGERLADIV |
| | | | | | LTPPEEHHAQPHALPPAEPSIAVEG |
| | | | | | LSFRYADGEPWVVKDCCFTIAPGES |
| | | | | | VAIIGGSGCGKTTLVKLLLGLLTPS |
| | | | | | EGTIRIGGHDLHKLGPRNVRAMIGV |
| | | | | | VMQDDQLFAGSIADNIGFFDTDFDL |
| | | | | | ERIEAAAQLAAVHEDIAAMPMGYHS |
| | | | | | LIGDMGSSLSGGQKQRIILARALYR |
| | | | | | QPKLLFLDEATSHLDLTRERLVNEA |
| | | | | | VKHLQLTKVIVAHRPETIASADRIL |
| | | | | | VMEHGRIVQEVKLQQSPEIPHPEDH |
| | | | | | VLSAMKAIVQAEASECGLASLAMVA |
| | | | | | SAHGMQLGLPELRRRFHLSLKGIRL |
| | | | | | NQLIEIAQTLGFSTRPLRLEMEQLD |
| | | | | | QLALPCFLHWDLNHFVVLAKVGKTK |
| | | | | | ATIL |
| 2 | WP_004086574.1 | peptidase | Xylella | 121 | MRSVLQSEVSECGLASLAMVANAHG |
| | | domain- | fastidiosa | | KHLTLSQLRQRFPSSLKGAKLTHLV |
| | | containing | | | HIAQQLGFSTRPLRLDMEDLGKLKL |
| | | ABC | | | PCILHWDLNHFVVLAKVGTTKVTIY |
| | | transporter | | | DPAIGKRKLSLNEVSKYFTGIALEL |
| | | | | | TTTTEFKQQKPPPSISARQLTGPIR |
| | | | | | GLWRALAQILLLSLALQVFVVLSPF |
| | | | | | YMQWVVDQVLVSADRDLLVVLGLGF |
| | | | | | GLVLLLQIAIGLFRSWSVIYLSSRL |
| | | | | | GLQWMGNVFAHLLKLPLEYFEKRHL |
| | | | | | GDLTSRMSSVQSIQKTLTTSFVEAI |
| | | | | | IDGLMTLVTLGLMLVYSWKLALVTL |
| | | | | | VAVVLYMVIRAVSYWPVRDCTEQQL |
| | | | | | VVGAKQQTHLLESLRGIQSLKVACE |
| | | | | | ESLRRSTYENLLNDTVNQEVRLARM |
| | | | | | SLGFSTASQLVFGLERIAVIWYGAT |
| | | | | | LALSNVFSVGMLIAYLSYKDQFASR |
| | | | | | ISSLIDKWVEFRMLRLHGERLADIV |
| | | | | | LTPPEEDYELPEMQLPKKEPDIEVE |
| | | | | | GLSFRYGEDEPWVLKDCSFKVAAGE |
| | | | | | SVAIIGASGCGKTTLMKLLLGLLKP |
| | | | | | TAGTIRISGRDLHKFGTRNVRSIVG |
| | | | | | AVMQDDQLFAGSVADNISFFDPDFD |
| | | | | | LERIEAAAKLAAIHEDIAAMPMGYH |
| | | | | | ALIGDMGSALSGGQKQRVILARALY |
| | | | | | RQPKLLFLDEATSHLDVMRERLVND |
| | | | | | AVHQLKLTKVIVAHRPETIASTDRV |
| | | | | | VILDHGRIVQTVAMPEDRDDSIQIV |
| | | | | | HNTIQST |
| 2 | WP_004090222.1 | peptidase | Xylella | 122 | MRSVLQSEVSECGLASLAMVANAHG |
| | | domain- | fastidiosa | | KHLTLSQLRQRFPSSLKGAKLTHLV |
| | | containing | | | HIAQQLGFSTRPLRLDMEDLGKLKL |
| | | ABC | | | PCILHWDLNHFVVLAKVGTTKVTIY |
| | | transporter | | | DPAIGKRKLSLNEVSKYFTGIALEL |
| | | | | | TTTTEFKQQKPPPSISARQLTGPIR |
| | | | | | GLWRALAQILLLSLALQVFVVLSPF |
| | | | | | YMQWVVDQVLVSADRDLLVVLGLGF |
| | | | | | GLVLLLQIAIGLFRSWSVIYLSSRL |
| | | | | | GLQWMGNVFAHLLKLPLEYFEKRHL |
| | | | | | GDLTSRMSSVQSIQKTLTTSFVEAI |
| | | | | | IDGLMTLVTLGLMLVYSWKLALVTL |
| | | | | | VAVVLYMVIRAVSYWPVRDCTEQQL |
| | | | | | VVGAKQQTHLLESLRGIQSLKVACE |
| | | | | | ESLRRSTYENLLNDTVNQEVRLARM |
| | | | | | SLGFSTASQLVFGLERIAVIWYGAT |
| | | | | | LALSNVFSVGMLIAYLSYKDQFASR |
| | | | | | ISSLIDKWVEFRMLRLHGERLADIV |
| | | | | | LTPPEEDYELPEMQLQKKEPDIEVE |
| | | | | | GLSFRYGEDEPWVLKDCSFKVAAGE |
| | | | | | SVAIIGASGCGKTTLMKLLLGLLKP |
| | | | | | TAGTIRISGRDLHKFGTRNVRSIVG |
| | | | | | AVMQDDQLFAGSVADNISFFDPDFD |
| | | | | | LERIEAAAKLAAIHEDIAAMPMGYH |
| | | | | | ALIGDMGSALSGGQKQRVILARALY |
| | | | | | RQPKLLFLDEATSHLDVMRERLVND |
| | | | | | AVHQLKLTKVIVAHRPETIASTDRV |
| | | | | | VILDHGRIVQTVAMPEDRDDSIEIV |
| | | | | | HNTIQST |
| 2 | WP_010893728.1 | peptidase | Xylella | 123 | MRPVLQSEVSECGLASLAMVANAHG |
| | | domain- | fastidiosa | | KHLTLSQLRQRFPSSLKGAKLTHLV |
| | | containing | | | HIAQQLGFSTRPLRLDMEDLGKLKL |
| | | ABC | | | PCILHWDLNHFVVLAKVGTTKVTVY |
| | | transporter | | | DPAIGKRKLSLNEVSKYFTGIALEL |
| | | | | | TTTTEFKQQKPPPSISARQLTGPIR |
| | | | | | GLWRALAQILLLSLALQVFVVLSPF |
| | | | | | YMQWVVDQVLVSADRDLLVVLGLGF |
| | | | | | GLVLLLQIAIGLFRSWSVIYLSSRL |
| | | | | | GLQWMGNVFAHLLKLPLEYFEKRHL |
| | | | | | GDLTSRMSSVQSIQKTLTTSFVEAI |
| | | | | | IDGLMTLVTLGLMLVYSWKLALVTL |
| | | | | | VAVALYMVIRAVSYWPVRDCTEQQL |
| | | | | | VVGAKQQTHLLESLRGIQSLKVACE |
| | | | | | ESLRRSTYENLLNDTVNQEVRLARM |
| | | | | | SLGFSTASQLVFGLERIAVIWYGAT |
| | | | | | LALSNVFSVGMLIAYLSYKDQFASR |
| | | | | | ISSLIDKWVEFRMLRLHGERLADIV |
| | | | | | LTPPEEDYELPEMQLPKKEPDIEVE |
| | | | | | GLSFRYGEDEPWVLKDCSFKVAAGE |
| | | | | | SVAIIGASGCGKTTLMKLLLGLLKP |
| | | | | | TAGTIRISGRDLHKFGTRNVRSIVG |
| | | | | | AVMQDDQLFAGSVADNISFFDPDFD |
| | | | | | LERIEAAAKLAAIHEDIAAMPMGYH |
| | | | | | ALIGDMGSALSGGQKQRVILARALY |
| | | | | | RQPKLLFLDEATSHLDVMRERLVND |
| | | | | | AVHQLKLTKVIVAHRPETIASTDRV |
| | | | | | VILDHGRIVQTVAMPEDRDDSIEIV |
| | | | | | HNTIQST |
| 3 | H47_PCAT_mchF | peptidase | Escherichia | 124 | MTNGSFRQIINQLDMRWRRRVPVIH |
| | | domain- | coli | | QTETAECGLACLAMICGHFGKNIDL |
| | | containing | | | ISLRRKENLSARGANLAGINGIAEQ |
| | | ABC | | | LGMVTRALSLELDELGALKMPCILH |
| | | transporter | | | WDFSHFVVLVSVKRNRYVLHDPARG |
| | | | | | RRYLGREEMSRYFTGIALEVWPGSE |
| | | | | | FLAETQQIRISLRSLINSIYGIKRT |
| | | | | | LAKIFCLSVVIEAINLVMPVGTQLV |
| | | | | | MDHAIPAGDRGLLTLISAGLMFFIL |
| | | | | | LRAAVSMLRAWSSLVMSTLINIQWQ |
| | | | | | SGLFNHLLRLPLAFFERRKLGDIQS |
| | | | | | RFGSLDTLRATFTTCVVGAIMDSIM |
| | | | | | VVGVFVMMLLYGGYLTWIVLGFTMV |
| | | | | | YVLIRLVTYGYYRQISEETLVRGAR |
| | | | | | ASSYFMESLYGIATVKIQGMAGIRG |
| | | | | | THWLNLKIDAINSGIKLTKMDLLFG |
| | | | | | GINTFVAACDQVAILWLGASLVIDN |
| | | | | | QMTIGMFVAFGSFRGQFSDRVASLT |
| | | | | | SFLLQLRIMSLHNERIADIALHEKE |
| | | | | | EKKPEIEIVADMSPVSLETTDLSYR |
| | | | | | YDSQSAQVFSGLNLSVAPGESVAIT |
| | | | | | GASGAGKTTLMKVLCGLFEPDSGKV |
| | | | | | LVNGTDIRQLGINNYHRMIACVMQD |
| | | | | | DRLFSGSIRENICGFAEETDDEWMT |
| | | | | | ECARASHIHDVIMKMPMGYETLIGE |
| | | | | | LGEGLSGGQKQRIFIARALYRKPGI |
| | | | | | LFMDEATSSLDTESERFVNAAIKKM |
| | | | | | NITRVIIAHRETTLRTVDRIISI |
| 3 | WP_001183600.1 | microcin | Escherichia | 125 | MQQDRSMTNGNFRQIINQLDMRWRR |
| | | export | coli | | RVPVIHQTETAECGLACLAMICGHF |
| | | transporter | | | GKNIDLISLRRKFNLSARGTNLAGI |
| | | peptidase/A | | | NGIAEQLGMVTRALSLELDELGALK |
| | | TP-binding | | | MPCILHWDFSHFVVLVSVKRNRYVL |
| | | subunit | | | HDPARGRRYLGREEMSRYFTGIALE |
| | | MchF | | | VWPGSEFLAETQQTRISLRSLINSI |
| | | | | | YGIKRTLAKIFCLSVVIEAINLLMP |
| | | | | | VGTQLVMDHAIPAGDRGLLTLISAG |
| | | | | | LMFFILLRAAVSMLRAWSSLVMSTL |
| | | | | | INIQWQSGLFNHLLRLPLAFFERRK |
| | | | | | LGDIQSRFGSLDTLRATFTTCVVGA |
| | | | | | IMDSIMVVGVFVMMLLYGGYLTWIV |
| | | | | | LGFTMVYVLIRLVTYGYYRQISEET |
| | | | | | LVRGARASSYFMESLYGIATVKIQG |
| | | | | | MAGIRGTHWLNLKIDAINSGIKLTR |
| | | | | | MDLLFGGINTFVAACDQVAILWLGA |
| | | | | | SLVIDNQMTIGMFVAFGSFRGQFSD |
| | | | | | RVASLTSFLLQLRIMSLHNERIADI |
| | | | | | ALHEKEEKKPEIEIVADMSPVSLET |
| | | | | | TDLSYRYDSQSAQVFSGLNLSVAPG |
| | | | | | ESVAITGASGAGKTTLMKVLCGLFE |
| | | | | | PDSGKVLVNGTDIRQLGINNYHRMI |
| | | | | | ACVMQDDRLFSGSIRENICGFAEET |
| | | | | | DDEWMTECAKASHIHDVIMKMPMGY |
| | | | | | ETLIGELGEGLSGGQKQRIFIARAL |
| | | | | | YRKPGILFMDEATSSLDTESERFVN |
| | | | | | AAIKKMNITRVIIAHRETTLRTVDR |
| | | | | | IFRITN |
| 3 | WP_004206412.1 | MULTISPECIES: | Klebsiella | 126 | MTNELFEKIITKVNFSLLKKTPVIL |
| | | peptidase | | | QSEAAECGIACLAMVCGHYGLDIDL |
| | | domain- | | | FNFRQRFGSPSQGVTLMSLSKTAEH |
| | | containing | | | AGLKSRALSLDLDEIRQLKLPCVLH |
| | | ABC | | | WGMNHYVVLTKVRKSSFVVHDPALG |
| | | transporter | | | KRIIGNQEMSNYFTGIALELWPDQN |
| | | | | | FQQEKAKSRLRLLDLMRNIVGLKSA |
| | | | | | LLKIFAFSVVVEAIGLLLPIGTQLV |
| | | | | | TDHVIMAHDQSLLSVICIGLVFFTL |
| | | | | | FRTFISMLRAWTSLTLNTLTNIQWK |
| | | | | | TTLFDHLTSLPLSFFEKRHLGDIQS |
| | | | | | RFSSLDTIRSTFTNSIVSGIIDSIM |
| | | | | | TIGLLIMLTLYGGWLTWVVVGFTLC |
| | | | | | YAIMRLATYHFYRRVAEEQVIKGAR |
| | | | | | SSSHFMESLYGISTIKALNLKERRS |
| | | | | | QHWLNINIEACNAGIKQTRFDMMFG |
| | | | | | GVNTFITAIDQVAVLWLGAIMVIDN |
| | | | | | NMTLGMFMAFNSYRGQFSQRASSLV |
| | | | | | DLCMQLRMLSLHNERLSEIVFSEPE |
| | | | | | KELPAREVFSPDSGAKLEVKNLCYQ |
| | | | | | YDPFSQPIFSNLNITVEPGESVALI |
| | | | | | GPSGVGKTTLLKVMCGLLSPTSGDV |
| | | | | | LADNLDITKIGLNNYRQGTACVLQE |
| | | | | | DRLFSGSLIDNISGFQDNADLDFVM |
| | | | | | ECAKRCNIHDEIMKMPMGYETIVGE |
| | | | | | LGLGISGGQKQRILIARALYRKPSI |
| | | | | | LFMDEATSHLDLRNESVINQSISAL |
| | | | | | SITRIIVAHRPSTIASADRIIDLSQ |
| | | | | | SKTLVPA |
| 3 | WP_032736707.1 | MULTISPECIES: | Klebsiella | 127 | MTNELFKTVVAKLNFSLINKTPVIL |
| | | peptidase | | | QSESSECGIACLAMVCGYYGLNIDL |
| | | domain- | | | FNFRQRFGSPSQGSSLMSLSKTAEH |
| | | containing | | | AGLKSRALALDLDEIKQLKLPCVIH |
| | | ABC | | | WGMNHYVVLTKVRKNGFIIHDPSLG |
| | | transporter | | | KRIIGISEMSNNFTGVALELWPDHN |
| | | | | | FQQEEAKSRLRLLDLMHKIVGLKSA |
| | | | | | LIKIFAFSVVVEAIGLLLPIGTQLV |
| | | | | | TDHVIMAHDESLLSVICIGLIFFTL |
| | | | | | FRTFISMLRAWTSLTLNTLTNIQWK |
| | | | | | TTLFDHLASLPLSFFEKRHLGDIQS |
| | | | | | RFSSLDTIRSIFTNSIVTGIIDSIM |
| | | | | | TIGLLVMLTLYGGWLVWVVVGFTVC |
| | | | | | YAIMRFATYGFYRRVTEEQVVKGAR |
| | | | | | ANSHFMESLYGIATIKALNLKGRRS |
| | | | | | QHWLNLNVDASNAGIKQTRFDMMYG |
| | | | | | GINTFITSIDQVAVLWLGAIMVIDN |
| | | | | | EMTLGMFMAFNAYRGQFSQRASNLI |
| | | | | | DLVMQLRMLSLHNERLSEIAFSEPE |
| | | | | | KELPSRRVFTENTGVQLEVRNLSYQ |
| | | | | | YDPFSQPIFTNLNITIAPGESVALI |
| | | | | | GPSGVGKTTLLKVMCGLLSPDSGEI |
| | | | | | LADNLEIKKIGLNNYRLGIACVLQE |
| | | | | | DRLFSGSLIDNISGFDDNVDLDFVM |
| | | | | | ECAKRCNIHDEIMKMAMGYETIVGE |
| | | | | | LGLGISGGQKQRILIARALYRKPSI |
| | | | | | LFMDEATSHLDLRNESVINQSISSL |
| | | | | | SITRIIVAHRPSTIASADRVIDLSQ |
| | | | | | SKSMTTA |
| 3 | WP_040971183.1 | MULTISPECIES: | Klebsiella | 128 | MINELFDKITSRVNFSLRRKAPIIL |
| | | peptidase | | | QSEGSECGIACLAMICGYHGLEIDL |
| | | domain- | | | FNFRHRFGSATQGATLLSLSKTAEQ |
| | | containing | | | AQLKSRALSLDLDEIQQLKLPCILH |
| | | ABC | | | WDLNHYVVLIKRKKNSFIIHDPAMG |
| | | transporter | | | KRVIGIKEMSNHFTGVALELWPDNN |
| | | | | | FQQEKAKSRLRLIDLMKNISGLKNA |
| | | | | | LLKIFAYSVVVEAINLLLPIGTQLV |
| | | | | | TDHVIIAHDESLLAVICIGLVMFTL |
| | | | | | FRTFISMLRAWTSLTLNTLTSIQWK |
| | | | | | TTLFDHLMSLPLSFFEKRHLGDIQS |
| | | | | | RFSSLDTIRSTFTNSIISGLIDLIM |
| | | | | | TVGLIIMLTLYGGWLVWVVIGFTLC |
| | | | | | YAIMRLATYQFYRRVAEEQVVKGAR |
| | | | | | SSSHFMESLYGISTIKALDLKGRRS |
| | | | | | EHWLNINIEACNAGIKQTRFDMLFG |
| | | | | | GINTLITSIDQVVILWLGAVMVIDN |
| | | | | | EMTLGMFMAFNAFRGQFSQRASSLV |
| | | | | | DLSMQLRMLSLHNERLSEIVFTEPE |
| | | | | | KELPARRIFEENRGVALNVQNLSYQ |
| | | | | | YDPFTKPIFSDLNINVLPGESIALV |
| | | | | | GPSGIGKTTLLKVMCGLLTPTHGEV |
| | | | | | VVEGLDISKIGLNNYRMSTACVLQE |
| | | | | | DRLFSGSIADNICSFEDNPDMEFVM |
| | | | | | HCAKLSNIHDEIMKMPMGYETLVGE |
| | | | | | LGLGISGGQKQRLLIARALYRKPNI |
| | | | | | LFMDEATSHLDLANESYINQSISSL |
| | | | | | SITRIIVAHRPSTIASADRIIDLSS |
| | | | | | KNIHK |
| 3 | WP_043519268.1 | MULTISPECIES: | Klebsiella | 129 | MTNELFKTIVAKLNFSLVNKTPVIL |
| | | peptidase | | | QSESSECGVACLAMVCGYYGLNIDL |
| | | domain- | | | FNFRQRFGSPSQGSSLLSLSKTAEQ |
| | | containing | | | AGLKNRALALDLDEIKQLKLPCIIH |
| | | ABC | | | WGMNHYVVLTKVKKNGFVIHDPALG |
| | | transporter | | | KRIIGISEMSNNFTGVALELWPDHN |
| | | | | | FQQEEAKSRLRLLDLMHKIVGLKSA |
| | | | | | LIKIFAFSVVVEAIGLLLPIGTQLV |
| | | | | | TDHVIMAHDESLLSVICIGLIFFTL |
| | | | | | FRTFISMLRAWTSLTLNTLTNIQWK |
| | | | | | TTLFDHLASLPLSFFEKRHLGDIQS |
| | | | | | RFSSLDTIRSIFTNSIVTGIIDSIM |
| | | | | | TIGLLVMLTLYGGWLVWVVIGFTAC |
| | | | | | YAIMRFATYSFYRRVTEEQVVKGAR |
| | | | | | ANSHFMESLYGISTIKALNLKERRS |
| | | | | | QHWLNLNVDASNAGIKQTRFDMMFG |
| | | | | | GINTFITSIDQVVVLWLGAIMVIDN |
| | | | | | EMTLGMFMAFNAYRGQFSQRASNLI |
| | | | | | DLVMQLRMLSLHNERLSEIAFSEPE |
| | | | | | KELPSRRVFTANTGVKLEVRNLSYQ |
| | | | | | YDPFSQPIFTNLNITVAPGESVALI |
| | | | | | GPSGVGKTTLLKVMCGLLSPDSGEI |
| | | | | | LADNLEIQKIGLNNYRLGIACVLQE |
| | | | | | DRLFSGSLIDNISGFDDNPDLEFVM |
| | | | | | ECAKHCNIHDEIMKMAMGYETIVGE |
| | | | | | LGLGISGGQKQRILIARALYRKPSI |
| | | | | | LFMDEATSHLDLRNESVINQSISAL |
| | | | | | SITRIIVAHRPSTIASADRVIDLSQ |
| | | | | | SKSMATA |
| 3 | WP_032418168.1 | peptidase | Klebsiella | 130 | MTNELFKTVVAKLNFSLVNKTPVIL |
| | | domain- | pneumoniae | | QSESSECGIACLAMVCGYYGLNIDL |
| | | containing | | | FNFRQRFGSPSQGSSLMSLSKTAEH |
| | | ABC | | | AGLKSRALALDLDEIKQLKLPCIIH |
| | | transporter | | | WGMNHYVVLTKVRKNGFIIHDPALG |
| | | | | | KRVIGISEMSNNFTGVALELWPDHN |
| | | | | | FQQEEAKSRLRLLDLMHKIVGLKPA |
| | | | | | LIKIFAYSVVVEAIGLLLPIGTQLV |
| | | | | | TDHVIMAHDQSLLSVICIGLIFFTL |
| | | | | | FRMFISMLRAWTSLTLNTLTNIQWK |
| | | | | | TTLFDHLASLPLSFFEKRHLGDIQS |
| | | | | | RFSSLDTIRSIFTNSIVTGIIDSIM |
| | | | | | TIGLLVMLTLYGGWLVWVVVGFTVC |
| | | | | | YAIMRFATYGFYRRVTEEQVVKGAR |
| | | | | | ANSHFMESLYGISTIKALNLKERRS |
| | | | | | QHWLNLNVDASNAGIKQTRFDMMYG |
| | | | | | GINTFITSIDQVAVLWLGAIMVIDN |
| | | | | | EMTLGMFMAFNAYRGQFSQRASNLI |
| | | | | | DLVMQLRMLSLHNERLSEIAFSEPE |
| | | | | | KELPSRRVFTENTGVKLEVRNLSYQ |
| | | | | | YDPFSQPIFTNLNITVDPGESVALI |
| | | | | | GPSGVGKTTLLKVMCGLLSPDSGEI |
| | | | | | LADNLEIKKIGLNNYRFGIACVLQE |
| | | | | | DRLFSGSLIDNISGFDDNADLDFVM |
| | | | | | ECARRCNIHDEIMKMSMGYETIVGE |
| | | | | | LGLGISGGQKQRILIARALYRKPSI |
| | | | | | LFMDEATSHLDLRNESVINQSISAL |
| | | | | | SITRIIVAHRPSTIASADRVIDLAQ |
| | | | | | SKPVTTA |
| 3 | WP_032439554.1 | peptidase | Klebsiella | 131 | MTNELFKTVVAKLNFSLVNKTPVIL |
| | | domain- | pneumoniae | | QSESSECGIACLAMVCGYYGLNIDL |
| | | containing | | | FNFRQRFGSPSQGSSLMSLSKTAEH |
| | | ABC | | | AGLKSRALALDLDEIKQLKLPCIIH |
| | | transporter | | | WGMNHYVVLTKVRKNGFIIHDPALG |
| | | | | | KRVIGISEMSNNFTGVALELWPDHN |
| | | | | | FQQEEAKSRLRLLDLMHKIVGLKPA |
| | | | | | LIKIFAYSVVVEAIGLLLPIGTQLV |
| | | | | | TDHVIMAHDQSLLSVICIGLIFFTL |
| | | | | | FRMFISMLRAWTSLTLNTLTNIQWK |
| | | | | | TTLFDHLASLPLSFFEKRHLGDIQS |
| | | | | | RFSSLDTIRSIFTNSIVTGIIDSIM |
| | | | | | TIGLLVMLTLYGGWLVWVVVGFTVC |
| | | | | | YAIMRFATYGFYRRVTEEQVVKGAR |
| | | | | | ANSHFMESLYGISTIKALNLKERRS |
| | | | | | QHWLNLNVDASNAGIKQTRFDMMYG |
| | | | | | GINTFITSIDQVAVLWLGAIMVIDN |
| | | | | | EMTLGMFMAFNAYRGQFSQRASNLI |
| | | | | | DLVMQLRMLSLHNERLSEIAFSEPE |
| | | | | | KELPSRRVFTENTGVKLEVRNLSYQ |
| | | | | | YDPFSQPIFTNLNITVDPGESVALI |
| | | | | | GPSGVGKTTLLKVMCGLLSPDSGEI |
| | | | | | LADNLEIKKIGLNNYRLGIACVLQE |
| | | | | | DRLFSGSLIDNISGFDDNADLDFVM |
| | | | | | ECARRCNIHDEIMKMSMGYETIVGE |
| | | | | | LGLGISGGQKQRILIARALYRKPSI |
| | | | | | LFMDEATSHLDLRNESVINQSISAL |
| | | | | | SITRIIVAHRPSTIASADRVIDLAQ |
| | | | | | SKPVTTA |
| 3 | WP_153932547.1 | peptidase | Klebsiella | 132 | MTNELFKTVVAKLNFSLVNKTPVIL |
| | | domain- | pneumoniae | | QSESSECGIACLAMVCGYYGLNIDL |
| | | containing | | | FNFRQRFGSPSQGSSLMSLSKTAEH |
| | | ABC | | | AGLKSRALALDLDEIKQLKLPCIIH |
| | | transporter | | | WGMNHYVVLTKVRKNGFIIHDPALG |
| | | | | | KRVIGISEMSNNFTGVALELWPDHN |
| | | | | | FQQEEAKSRLRLLDLMHKIVGLKPA |
| | | | | | LIKIFAYSVVVEAIGLLLPIGTQLV |
| | | | | | TDHVIMAHDQSLLSVICIGLIFFTL |
| | | | | | FRMFISMLRAWTSLTLNTLTNIQWK |
| | | | | | TTLFDHLASLPLSFFEKRHLGDIQS |
| | | | | | RFSSLDTIRSIFTNSIVTGIIDSIM |
| | | | | | TIGLLVMLTLYGGWLVWVVVGFTVC |
| | | | | | YAIMRFATYGFYRRVTEEQVVKGAR |
| | | | | | ANSHFMESLYGISTIKALNLKERRS |
| | | | | | QHWLNLNVDASNAGIKQTRFDMMYG |
| | | | | | GINTFITSIDQVAVLWLGAIMVIDN |
| | | | | | EMTLGMFMAFNAYRGQFSQRASNLI |
| | | | | | DLIMQLRMLSLHNERLSEIAFSEPE |
| | | | | | KELPSRRVFTENTGVKLEVRNLSYQ |
| | | | | | YDPFSQPIFTNLNITVDPGESVALI |
| | | | | | GPSGVGKTTLLKVMCGLLSPDSGEI |
| | | | | | LADNLEIKKIGLNNYRLGIACVLQE |
| | | | | | DRLFSGSLIDNISGFDDNADLDFVM |
| | | | | | ECARRCNIHDEIMKMSMGYETIVGE |
| | | | | | LGLGISGGQKQRILIARALYRKPSI |
| | | | | | LFMDEATSHLDLRNESVINQSISAL |
| | | | | | SITRIIVAHRPSTIASADRVIDLAQ |
| | | | | | SKPVTTA |
| 3 | WP_012969129.1 | peptidase | Klebsiella | 133 | MTNELFKTVVAKLNFSLINKTPVIL |
| | | domain- | variicola | | QSESSECGIACLAMVCGYYGLNIDL |
| | | containing | | | FNFRQRFGSPSQGSSLMSLSKTAEH |
| | | ABC | | | AGLKSRALALDLDEIKQLKLPCVIH |
| | | transporter | | | WGMNHYVVLTKVRKNGFIIHDPSLG |
| | | | | | KRIIGISEMSNNFTGVALELWPDHN |
| | | | | | FQQEEAKSRLRLLDLMHKIVGLKSA |
| | | | | | LIKIFAFSVVVEAIGLLLPIGTQLV |
| | | | | | TDHVIMAHDESLLSVICIGLIFFTL |
| | | | | | FRTFISMLRAWTSLTLNTLTNIQWK |
| | | | | | TTLFDHLASLPLSFFEKRHLGDIQS |
| | | | | | RFSSLDTIRSIFTNSIVTGIIDSIM |
| | | | | | TIGLLVMLTLYGGWLVWVVVGFTVC |
| | | | | | YAIMRFATYGFYRRVTEEQVVKGAR |
| | | | | | ANSHFMESLYGIATIKALNLKGRRS |
| | | | | | QHWLNLNVDASNAGIKQTRFDMMYG |
| | | | | | GINTFITSIDQVAVLWLGAIMVIDN |
| | | | | | EMTLGMFMAFNAYRGQFSQRASNLI |
| | | | | | DLVMQLRMLSLHNERLSEIAFSEPE |
| | | | | | KELPSRRVFTENTGVQLEVRNLSYQ |
| | | | | | YDPFSQPIFTNLNITIAPGESVALI |
| | | | | | GPSGVGKTTLLKVMCGLLSPDSGEI |
| | | | | | LADNLEIKKIGLNNYRLGIACVLQE |
| | | | | | DRLFSGSLIDNISGFDDNVDLDFVM |
| | | | | | ECAKRCNIHDEIMKMAMGYETIVGE |
| | | | | | LGLGISGGQKQRILIARALYRKPSI |
| | | | | | LFMDEATSHLDLRNESVINQSISAL |
| | | | | | SITRIIVAHRPSTIASADRVIDLSQ |
| | | | | | SKSVTTA |
| 3 | WP_064563844.1 | peptidase | Kosakonia | 134 | MNKDILDSIFKRINFSIKNKVPVII |
| | | domain- | oryzae | | QSEATECGNACLAMICGYYGKDIDL |
| | | containing | | | FNFRTHYGSPSQGATLTDLNNVALK |
| | | ABC | | | TGLKTRALSLDIDEIKELRLPCVLH |
| | | transporter | | | WSLNHFVVLVAVKGKRFVINDPAMG |
| | | | | | RRIVHQKEMSENFTGIALEAWPDSH |
| | | | | | FKQEKQRSRLKLLDLMRNIVGLKSA |
| | | | | | LSKIFMLSVVIEAINLLMPMGTQLV |
| | | | | | TDHVITAHDSNLLLVICAGLLFFTI |
| | | | | | FKTAVSMIRAWVSLKLNTLTDIQWK |
| | | | | | TSFFDHLLSLPLAFFEKRNLGDIQS |
| | | | | | RFSSLDTIRATFTNNIVMGTIDSIM |
| | | | | | TVGVLVMMSLYGGWLVWVVLGFTVC |
| | | | | | YAAMRIITYKFYRTISEELIVKRAR |
| | | | | | SGSHFMETLYGIATIKSLNLKNRRS |
| | | | | | QHWLNTNIDVSNASLRQTRFDMLFG |
| | | | | | GINTFINSVDQVVILWLGALMVMDN |
| | | | | | TMTIGMFMAFNAYRGQFAQRASSLI |
| | | | | | DLAMQFKMLSLHNERISEIVFSKPE |
| | | | | | AESPPRKVFENSAGVPLQVQNLSYQ |
| | | | | | YDKLTKPVFSNVNISVAAGESVALI |
| | | | | | GASGIGKTTLLKVMSGLLTPDHGDI |
| | | | | | FVGDFDVKKIGVNNYRACTACVLQE |
| | | | | | DRLFSGSIADNISGFEDNADQQLIV |
| | | | | | ECAIKCNIHEEIMRMPMGYETIIGE |
| | | | | | LGSGISGGQKQRLLIARALYQKPNI |
| | | | | | LFMDEATSHLDANNERMINNAIESL |
| | | | | | NITRIIVAHRPSTIACADRVIDLSK |
| | | | | | ISSL |
| 3 | WP_007370272.1 | peptidase | Kosakonia | 135 | MNKAIIDTIFKRINFSIRNKVPVII |
| | | domain- | radicincitans | | QSEAAECGNACLSMICGYYGKNIDL |
| | | containing | | | FNFRNRYGSPAQGATLTDLNQVAQT |
| | | ABC | | | TGLKTRALSLDMDEIKELRLPCVLH |
| | | transporter | | | WSLNHFVVLVAIKGKRFVINDPAMG |
| | | | | | RRVIHQKEMSENFSGIALEAWPDSH |
| | | | | | FTQEKQRSRLNLLDLMCNIVGLKSA |
| | | | | | LSKIFMLSIVIEAVNLLMPMGTQLV |
| | | | | | TDHVITAHDNNLLLVICAGLLFFTL |
| | | | | | FKTAVSMIRAWVSLKLNTLTDIQWK |
| | | | | | TSFFDHLLSLPLAFFEKRNLGDIQS |
| | | | | | RFSSLDTIRATFTNNIVMGTIDSIM |
| | | | | | TVGVLIMMSLYGGWLVWVVLGFTVC |
| | | | | | YAAMRIITYKFYRTISEELIVKRAR |
| | | | | | SGSHFMETLYGIATIKSLNLKNRRS |
| | | | | | QHWLNTNIDVSNASLRQTRFDMLFG |
| | | | | | GINTFINSVDQVVILWLGALMVMDN |
| | | | | | TMTIGMFMAFNAYRGQFAQRASSLI |
| | | | | | DLAMQFKMLSLHNERISEIIFSKPE |
| | | | | | AESPPRKVFEDSAGVPLQVQNLSYQ |
| | | | | | YDKLTRPVFSNVNITVAAGESVALT |
| | | | | | GASGIGKTTLLKVMSGLLTPDQGDI |
| | | | | | FVGDFDVKKIGVNNYRACTACVLQE |
| | | | | | DRLFSGSIADNISGFEDNADRQLII |
| | | | | | ECAIKCNIHEEIMRMPMGYETIIGE |
| | | | | | LGSGISGGQKQRLLIARALYQKPNI |
| | | | | | LFMDEATSHLDANNERMINNAIESL |
| | | | | | NITRIIVAHRPSTIACADRVIDLSK |
| | | | | | ISSL |
| 3 | WP_040080945.1 | peptidase | Mannheimia | 136 | MTGIKQLKFDFRRRIPVILQTETAE |
| | | domain- | haemolytica | | CGLACLAMMLGYYDKNTNLFELRSQ |
| | | containing | | | YGTSSRGVTLHTLISIANDCGLITR |
| | | ABC | | | PLSLELDEVPQLRLPCILHWDFNHF |
| | | transporter | | | VVLTHASEKQFIIHDPAFGKRKLSR |
| | | | | | AEFSNHFTGVALEVWSEVKFEKSNN |
| | | | | | ENTISLYETLKHISGIKGALVKIFA |
| | | | | | LSMLIELIALLMPIGTQLVMDHVMQ |
| | | | | | AKDQSLLLIICIGLFLFTLFRTMVS |
| | | | | | MFRAWVSLKMNYLIDFQWTASFFSH |
| | | | | | LLKLPLDFFEKRQVGDIHSRFSSLK |
| | | | | | TIQKTLTSSIITTIIDTIMIISLII |
| | | | | | MMVLYGGWLFWVVLAFSFIYFILRM |
| | | | | | VTYLVYRQLNEEQIIKGAKAHSHFM |
| | | | | | ETLYGITTLKSLGLENKRQEQWMSL |
| | | | | | NADAFNTSIKVTKFDMLFSGIHTFI |
| | | | | | STIEQILILWIGATMVIENVMTLGM |
| | | | | | FVAFNAYRGSFSSRMGNLIDIIFSL |
| | | | | | KILSLHRERIADIALNEVEDESKYQ |
| | | | | | ELRFEHNQTDIEVKDLCFKYDPFSK |
| | | | | | NVLENVNLVIRSGESVAITAPSGYG |
| | | | | | KTTLLKLIAGLLKPTSGSVYFNGID |
| | | | | | IYQLGLSQYRSQIACVLQEDKFFSG |
| | | | | | SILENIVSFESNYDREWAIECSKLA |
| | | | | | NIHNDIMAMPMNYETLLGELGNNLS |
| | | | | | GGQRQRLFIARALYKKPKILLMDEA |
| | | | | | TSHLDEENEKLVNQAISRMKITRII |
| | | | | | IAHRQSTIQSADRIIALNDR |
| 3 | WP_004237687.1 | peptidase | Morganella | 137 | MRRLSLKNLLEKLDLSLRQRIPVIH |
| | | domain- | morganii | | QTESSECGLASLAMISAHYGKSIDL |
| | | containing | | | ISLRQQFNLSARGATLAGLTGMAAE |
| | | ABC | | | LGMTTRALSLDMDDLPNLRLPCILH |
| | | transporter | | | WDFNHFLVLVKISGNKFILHDPAYG |
| | | | | | RRVVGIEEMSRNFTGVALEAWPGST |
| | | | | | FTRQEVVKKLSLRKLIGNIHGLKKA |
| | | | | | LLKIFAFSVVIEAIGLIMPVGTQLV |
| | | | | | MDHAIPAGDQGLLSLICVGLLFFIL |
| | | | | | LKAAVSMCRAWASVIMETLINVQWQ |
| | | | | | SGLFTHLLRLPLSYFERRKLGDIQS |
| | | | | | RFGSLDILRTTFTTSVVGAIMDGIM |
| | | | | | LIGVFIMMILYGGHLTWIVLGFTAV |
| | | | | | YIGLRLATYRYYRQLSEESLIKEAR |
| | | | | | AGSYFMESLYGITTVKTQGMSDRRG |
| | | | | | NHWLNLKVDSINTGIRLTKMNLMFG |
| | | | | | GVNIFIMACDQIAILWIGAGLVIDN |
| | | | | | AMTLGMFVAFSAYREQFSDRAASII |
| | | | | | EFLLQLRIMSLHNERISDIALNEQE |
| | | | | | NKKPDVPYEPQMQAAGLETRNLAYS |
| | | | | | YDSQSVPIFRDINLSVAPGESVAIT |
| | | | | | GPSGSGKTTLMKVLCGLFEPSDGKV |
| | | | | | FIDNIEIRQLGVNNYHKMIACVMQD |
| | | | | | DKLFSGSIRENICGFTENPDEQLMI |
| | | | | | ACAQASYIHNVIISMPMGYETLIGE |
| | | | | | LGEGLSGGQKQRIFIARALYRKPRI |
| | | | | | LFMDEATSALDKESESYVNQAIRQL |
| | | | | | PITRIIIAHRESTIESADRIISLGG |
| | | | | | KNNK |
| 3 | WP_036412653.1 | peptidase | Morganella | 138 | MRRLSLKNLLEKLDLSLRQRIPVIH |
| | | domain- | morganii | | QTESSECGLASLAMISAHYGKSIDL |
| | | containing | | | ISLRQQFNLSARGAALAGLTGMAAE |
| | | ABC | | | LGMTTRALSLDMDDLPNLRLPCILH |
| | | transporter | | | WDFNHFLVLVKISGNKFILHDPAYG |
| | | | | | RRVVGIEEMSRNFTGVALEAWPGST |
| | | | | | FTRQEVVKKLSLRKLIGNIHGLKKA |
| | | | | | LLKIFAFSVVIEAIGLIMPVGTQLV |
| | | | | | MDHAIPAGDQGLLSLICVGLLFFIL |
| | | | | | LKAVVSMCRAWASVIMETLINVQWQ |
| | | | | | SGLFTHLLRLPLSYFERRKLGDIQS |
| | | | | | RFGSLDILRTTFTTSVVGAIMDGIM |
| | | | | | LIGVFIMMILYGGHLTWIVLGFTAV |
| | | | | | YIGLRLATYRYYRQLSEESLIKEAR |
| | | | | | AGSYFMESLYGITTVKTQGMSDRRG |
| | | | | | NHWLNLKVDSINTGIRLTKMNLMFG |
| | | | | | GVNTFIMACDQIAILWIGAGLVIDN |
| | | | | | AMTLGMFVAFSAYREQFSDRAASII |
| | | | | | EFLLQLRIMSLHNERISDIALNEQE |
| | | | | | NKKPDVPYEPQMQAAGLETRNLAYS |
| | | | | | YDSQSAPIFRDINLSVAPGESVAIT |
| | | | | | GPSGSGKTTLMKVLCGLFEPSGGKV |
| | | | | | FIDNIEIRQLGVNNYHKMIACVMQD |
| | | | | | DKLFSGSIRENICGFTENPDEQLMI |
| | | | | | ACAQASYIHNVIISMPMGYETLIGE |
| | | | | | LGEGLSGGQKQRIFIARALYRKPRI |
| | | | | | LFMDEATSALDKESESYVNQAIRQL |
| | | | | | PITRIIIAHRESTIESADRIISLGG |
| | | | | | KNNK |
| 3 | WP_150105811.1 | ATP- | Pantoea sp. | 139 | MPVLLQTEAAECGLVSLAMIASWYQ |
| | | binding | At-9b | | LPADLRFLRQQSGISSRGATLRTVI |
| | | cassette | | | DTAAVAGLKSRALRLEMDNLRALKL |
| | | domain- | | | PCILHWDLNHFVVLVAVKRNRVVIH |
| | | containing | | | DPAAGHRVLGMQDVSLHFTGVALEL |
| | | protein | | | WPSGEMPKTPPARPRIPLWSLTGRI |
| | | | | | AGLGRTLTKLFCYSLLIESVSLLLP |
| | | | | | VGMQMVLDHAIPASDAGLLTLICLG |
| | | | | | LVFFVLFRTGASMLRGWTSLVMSTL |
| | | | | | ITVQGKARLFDHLLALPTEWFEKRK |
| | | | | | LGDIQSRFTSLETLQTTLTSNVVSG |
| | | | | | IMDSIMTIGLLIMMLLYGGNLLWVV |
| | | | | | LAFTLLWVLFRLGTYQAWRQTSEEQ |
| | | | | | IVRNARSGSHFMETLYGIHTIKALG |
| | | | | | LPALRAQHWFTLNVESANTTIRKTR |
| | | | | | FEMLFTGGNTLIAALQQITLLWLGA |
| | | | | | NAVIAGHMTLGMFVAFSTYRGQFAE |
| | | | | | RTAALLDNVLQLRMLSLHKERVADI |
| | | | | | ILTEPETTPGEPVHPLLNQGEPAAL |
| | | | | | AVKGLTFRHDPFSPLLFNALDLEIA |
| | | | | | ARECVAITGPSGRGKTTLLKIMAGL |
| | | | | | LKPTAGEVYVNGIDIRASGIANWQA |
| | | | | | CIACVLQEDTLLAGSVAENICSFDG |
| | | | | | QPDDVWIQECARLSQIHDDILAMPM |
| | | | | | GYQTLISELGGSLSGGQKQRLLIAR |
| | | | | | ALYRRPQVLFLDEATSHLDVENESR |
| | | | | | INAAIRGLNITRVMIAHRPSTIASA |
| | | | | | DRVIDMAEWS |
| 3 | WP_065506875.1 | peptidase | Serratia | 140 | MNKTYEKIVSKLNLAIKRNVPQILQ |
| | | domain- | inhibens | | TEAAECGLASLAMVCGYYGMHIDLF |
| | | containing | | | ALRQKHDISSRGATLGSLITIAENL |
| | | ABC | | | NLKTRALSLSLDEIRQVKTPCILHW |
| | | transporter | | | DMNHFVVLVNVRGGRITLHDPAFGR |
| | | | | | RVIGLQEFSLHFTGIALEVWPSSDF |
| | | | | | SPVVQQSRLHFRKLLKNVTGLKSAL |
| | | | | | GKIFALSLVIEAINLLMPVGTQLVM |
| | | | | | DHVIQAGDHNLLVLICVGLLFFIIF |
| | | | | | RTVVSMFRSWISIVMGALIDIQWKA |
| | | | | | GLFDHLMRLPLAYFEKRKLGDIQSR |
| | | | | | FGSLDALRTTFTTSVVSSIIDGIMS |
| | | | | | IGVFIMMFMYGGWLVWVVTGFTLLY |
| | | | | | ILLRLSTYRYYRQASEEQLVKAAKA |
| | | | | | SSHFMETLYSIATLKSLGLGGTRSQ |
| | | | | | FWLNLNIDTTNANIRVTKLDMMFGG |
| | | | | | VNAFLTACDQIIILWLGASLVIDNQ |
| | | | | | MTLGMFVAFNAYRGQFSDRASNLID |
| | | | | | MVLRLRMLNLHNERLADIVLSTPEE |
| | | | | | DKPYRKICAPNEAVTFEVRDLYYQY |
| | | | | | DSLSRPVIPGLNLNIAAGESVAIVG |
| | | | | | PSGVGKTTLMKLMCGLLEPTGGRLL |
| | | | | | INGINISDIGINNYRECIACVLQED |
| | | | | | KLLAGSIAENICSFDPKPDQDLIVE |
| | | | | | CTKHCNIHNDIIRMPMGYETLVGEL |
| | | | | | GGSLSGGQKQRILIARALYRRPSIL |
| | | | | | FMDEATSHLDLDNETYINNAISALN |
| | | | | | ITRVIIAHRPSTISSADRVITLKSS |
| | | | | | IQEE |
| 3 | WP_033638474.1 | peptidase | Serratia | 141 | MTESYFDTLKGKLNLSLRRKVPQIL |
| | | domain- | marcescens | | QTEASECGLASLAMVCHYHGLQIDL |
| | | containing | | | FNLRSRYGFSSRGATLSALIDIASA |
| | | ABC | | | LKLQSRALSLNIDELKALKMPCILH |
| | | transporter | | | WDMKHFVVLVSVSRGRAVIHDPAFG |
| | | | | | RKVLGLHELSRHFTGVALELWPDSE |
| | | | | | FQPIKQQSRLRFRKLMSNVRGLKGA |
| | | | | | LLKIFCLSIVIETVNLLLPVGTQLV |
| | | | | | MDHVILAGDHDLLALICIGLLFFIL |
| | | | | | FRTGVSMLRSWISIVMGALIDVQWK |
| | | | | | AGVFDHLMKLPLSFFEKRKLGDIQS |
| | | | | | RFGSLDTIRATFTTSIVSSIIDGIM |
| | | | | | SVGVFIMMLLYGGWLVWVVLGFTAI |
| | | | | | YVILRLSTYQYYRQLSEEQLVKGAR |
| | | | | | AGSHFMETLYSVSTLKALGLSETRS |
| | | | | | QYWLNLNVETINAGIKLTKLNMMFG |
| | | | | | GLGAFIATCDQVIILWLGAALVIDN |
| | | | | | QMTIGMFIAFNAYRGQFSERASSLI |
| | | | | | DMMLQLRMLSLHNERVADIVLSSPE |
| | | | | | SQMPARQLFTKGKAAELQVRNLRYQ |
| | | | | | YDRLAKPIIADLNLSIAAGESVAIV |
| | | | | | GPSGAGKTTLMKLMCGLLSPDEGAV |
| | | | | | LVDGMDISNIGVNNYRQCIACVLQD |
| | | | | | DKLLAGSIAENISGFDAEMDLKRIE |
| | | | | | ACARRCNMHDDIQNMPMGYETLIGE |
| | | | | | LGGSLSGGQKQRLLIARALYRRPSI |
| | | | | | LFMDEATSHLDLDNETHINRAISQL |
| | | | | | NITRVIIAHRPSTIASADRVIRLG |
| 3 | WP_063989775.1 | peptidase | Serratia | 142 | MTESYFDALKGKLNLSLRRKVPQIL |
| | | domain- | marcescens | | QTEASECGLASLAMVCHYHGLQIDL |
| | | containing | | | FNLRSRYGFSSRGATLAALIDIASA |
| | | ABC | | | LKLQSRALSLNIDELKALKMPCILH |
| | | transporter | | | WDMKHFVVLVSVSRGRAVIHDPAFG |
| | | | | | RKVLGLHELSRHFTGVALELWPDSE |
| | | | | | FQPIKQQSRLRFRKLMSNVRGLKGA |
| | | | | | LLKIFCLSIVIEAVNLLLPVGTQLV |
| | | | | | MDHVILAGDHDLLALICIGLLFFIL |
| | | | | | FRTGVSMLRSWISIVMGALIDVQWK |
| | | | | | AGVFDHLMKLPLSFFEKRKLGDIQS |
| | | | | | RFGSLDTIRATFTTSIVSSIIDGIM |
| | | | | | SVGVFIMMLLYGGWLVWVVLGFTAI |
| | | | | | YVILRLSTYQYYRQLSEEQLVKGAR |
| | | | | | AGSHFMETLYSVSTLKALGLSETRS |
| | | | | | QYWLNLNVETINAGIKLTKLNMMFG |
| | | | | | GLGAFIATCDQVIILWLGAALVIDN |
| | | | | | QMTIGMFIAFNAYRGQFSERASSLI |
| | | | | | DMMLQLRMLSLHNERVADIVLSSPE |
| | | | | | SQMPARQLFTKGNAAELQVRNLRYQ |
| | | | | | YDRLAKPIIADLNLSIAAGESVAIV |
| | | | | | GPSGAGKTTLMKLMCGLLSPDEGAV |
| | | | | | LVDGMDISNIGVNNYRQCIACVLQD |
| | | | | | DKLLAGSIAENISGFDAEMDLKRIE |
| | | | | | ACARRCNMHDDIQNMPMGYETLIGE |
| | | | | | LGGSLSGGQKQRLLIARALYRRPSI |
| | | | | | LFMDEATSHLDLDNETHINRAISQL |
| | | | | | NITRVIIAHRPSTIASADRVIRLG |
| 4 | N_PCAT_mcnB | peptidase | Escherichia | 143 | MNNNATSPLNTLLNKLEIGLRRRIP |
| | | domain- | coli | | VVHQTESSECGLACLSMICGHYGRH |
| | | containing | | | IDLSTLRRQFNLSALGTTLAGITEI |
| | | ABC | | | GSQLGMETRAFSLDLNELSVLKLPC |
| | | transporter | | | ILHWEFSHFVVLVSVRKNHFVLHDP |
| | | | | | ARGRRTVGLAEMSQCFTGVALEVWP |
| | | | | | GTEFVQETMKNRVVLRTLFRSIYGL |
| | | | | | RSTLTKIFCFSLVIEAVGLVIPVGT |
| | | | | | QLVMDHAIPAGDRGLLSLICVGLMF |
| | | | | | FILLRTAVSMIRSWSSLVMETLINV |
| | | | | | QWQSGLHRHLLQLPLAYFERRKMGD |
| | | | | | IQSRFSSLDTLRTTFTTSVVGAIMD |
| | | | | | SIMVSGVLAMLVLYGGWLTTIVLGF |
| | | | | | TIIYVLIRLLTYNYYRQLSEESLIR |
| | | | | | EARASSYFMETLYGIATIKMQGMGE |
| | | | | | RRGRHWLNLKIDAINTGIRLARMDM |
| | | | | | LFSGINTFVAACDQVVILWLGTSLV |
| | | | | | IDNQMTIGMFVAFGVFRGQFSDRVG |
| | | | | | SLTNFLLQLRMMSLHNERIADIAMN |
| | | | | | EREARKPDTAMKADMYPVALETQDL |
| | | | | | SFRYDSQSAPVFSNLNISIKPGESV |
| | | | | | AITGASGSGKTTLMKVLCGLLVPES |
| | | | | | GRVMIDGTDIRSLGVNNYHKIISCV |
| | | | | | MQDDRLFSGSIRENICGFTENIDEA |
| | | | | | WMVECARASFIHDVIIKMPMGYDTL |
| | | | | | IGELGEGLSGGQKQRIFIARALYRR |
| | | | | | PGILFMDEATSALDTESEYYVNQAI |
| | | | | | KQLNITRIIIAHRETTVKSADRIIL |
| | | | | | LEAPARI |
| 4 | WP_032052373.1 | MULTISPECIES: | Acinetobacter | 144 | MLNFSLKRKVPVILQHESAECGLAC |
| | | peptidase | calcoaceticus/ | | LAMIVNYYGKSISIITLRNKYKISL |
| | | domain- | baumannii | | QGTSLNMLMIISDDLQLQTRPIKID |
| | | containing | complex | | IENLENINLPCILHWEFNHFVILTK |
| | | ABC | Bacteria | | INHNNYIIHDPAIGVRKINSEDFSK |
| | | transporter | | | SFTGIALEIWPKPNFNSQNNEYKIK |
| | | | | | KSNFNNFFNEIIGIKSVLVQIFFIS |
| | | | | | LSLELVILLNPQLLQWIIDQIIPTQ |
| | | | | | NESSLSILALSFVFILAFQVILDLA |
| | | | | | RENLNIFMKFNISLKWQANIVSKIL |
| | | | | | DLPVDFFEKRSIGDIASKFGSINTI |
| | | | | | LNTITSSFMTAIFDGIMSILILSLL |
| | | | | | LFYSPLLTSIVFIFTILYLVIKIVF |
| | | | | | YYPQKYKNEERIIHTAKQNSHFLES |
| | | | | | IRGVKTIKTFNAQIERKSIWFSLLT |
| | | | | | NQINSEIQVEKINIMTKEFRIFLFG |
| | | | | | LENIVIIYLATLLVLKNELSVGSLV |
| | | | | | AFVVYKTLFTNRITALIDKLSEIGT |
| | | | | | IKLHNDRLRDISLTEPEIIYPQEFM |
| | | | | | EINSNKLSIKVKNISFSYSNYEKSI |
| | | | | | LNNITFEIPYGQSVAITGASGSGKS |
| | | | | | TLLNILLGILAPTTGDVFYGEHKIK |
| | | | | | ELGLYNIRSLVGTVLQEDVLFSGSI |
| | | | | | ADNISFFDASSNLDWIQECAKMAAI |
| | | | | | HEEIINTSMGYNTLIGEMGTVLSGG |
| | | | | | QKQRILLARALYKRPKILFLDEATS |
| | | | | | NLDIYKEFEVNNLIKKLKITRFIIA |
| | | | | | HRQETINSADRVLIIEDGILRSDIM |
| | | | | | NS |
| | WP_019725134.1 | MULTISPECIES: | | 145 | MKSEVVSVLKNKLDLGLKSRFPMIH |
| | | peptidase | | | QTESSECGLACLAMICGYYGKNIDI |
| | | domain- | | | FTLRQKFNLSSRGTDLASLNDIAVK |
| | | containing | | | MHMSTRAVSVELDELHSVKLPCILH |
| | | ABC | | | WDFNHFVVLTKIKNGKFTIHDPSVG |
| | | transporter | | | IVKVAKSELSKKFTGIILEIWPDNN |
| | | | | | FLKENVKNKISLRAMLENTHGLYSA |
| | | | | | LTKIFFLSIIIESINLVMPVATQLV |
| | | | | | MDHAIPANDSGLLTLICFGLVFFVA |
| | | | | | LRTITGLIRSWTVLIMSSFINAQWQ |
| | | | | | KGLLQHLLKLPLEYFERRKFGDIQS |
| | | | | | RFGSLNTLQETFTTSIVGAIIDSIM |
| | | | | | IIGLLIMLVLYGGWLTWFVILFTCL |
| | | | | | YVFIRVLTYNRYRQLSEESLIKDAR |
| | | | | | ANSFFMETLYGIATVKVQGLNKRRE |
| | | | | | ENWFNLEVDSINTGIKVSRLNLFFG |
| | | | | | GINTLIATMEQILILWLGASLVISG |
| | | | | | NMTIGMFVAFSSYRGQFSDRIASFI |
| | | | | | DFLLRLQIMSLHNERVSDIALTEVS |
| | | | | | PFKDDIPVKNDMCPVSLSAKNISYK |
| | | | | | YDSYASYTLRHLNIEIQPGEHVAIT |
| | | | | | GASGTGKTTLLKVLCGLFPASEGNI |
| | | | | | LVDGIDIKELGINNYQKLIACVLQD |
| | | | | | DRLFSGTLRDNICSFSTQIDDNWLI |
| | | | | | ECARFAQIHDDIERMPMGYDTLIGE |
| | | | | | LGEGLSGGQKQRIFIARAIYKKPSI |
| | | | | | LFMDEATCSLDNTNEQYINDAIKSL |
| | | | | | NITRIVIAHRKSTINSADRIITLQE |
| 4 | | | | | NL |
| 4 | WP_004186613.1 | MULTISPECIES: | Enterobacteriaceae | 146 | MNNKLFETIIAKLNFSLIKKTPVIL |
| | | peptidase | | | QSESAECGIASLAMICGHYGLDIDL |
| | | domain- | | | FNFRQRFGSPSQGASLLHLSKTAER |
| | | containing | | | AGLKNRALSLDLDEIKQLKLPCIIH |
| | | ABC | | | WGMNHYVVLTKVRKSSYVVHDPALG |
| | | transporter | | | KRIIGLQEMSNNFTGVALELWPDQN |
| | | | | | FQQEKAKARLRLLDLMRNIVGLKSA |
| | | | | | LIKIFAFSVVIEAIGLLLPIGTQLV |
| | | | | | TDHVIMAHDQSLLSVICIGLVFFTL |
| | | | | | FRTFVSMLRAWTSLTLNTLTNIQWK |
| | | | | | TTLFDHLTSLPLSFFEKRHLGDIQS |
| | | | | | RFTSLDTIRSTFTNSIVSGIIDSIM |
| | | | | | TIGLLIMLTLYGGWLVWVVVGFTLC |
| | | | | | YAIMRLATYRFYRRVAEEQVIKGAR |
| | | | | | SSSHFMESLYGISTIKALNLKERRS |
| | | | | | QHWLNINIDACNAGIKQTRFDMMFS |
| | | | | | GINTFISSIDQVAILWLGAIMVIDN |
| | | | | | NMTLGMFMAFNSYRGQFSQRASNLI |
| | | | | | DLFMQLRMLSLHNERLSEIVFSEPE |
| | | | | | KELPARRVFDENSGVKLEIKNLSYQ |
| | | | | | YDPFSQPIFSNFNLQVEPGESIALV |
| | | | | | GPSGVGKTTLLKVMCGLLSPTSGEV |
| | | | | | IADNLDIYKIGLNNYRLGTACVLQE |
| | | | | | DRLFSGSISDNISGFEDNADEELIV |
| | | | | | ECARRSNIHDEIMKMPMGYETMIGE |
| | | | | | LGLGISGGQKQRLLIARALYRKPSI |
| | | | | | LFMDEATSHLDLKNESAINQSIASL |
| | | | | | SITRIIVAHRPSTIASADRIIDLSQ |
| | | | | | YGKQAS |
| 4 | WP_149283502.1 | peptidase | Halomonas sp. | 147 | MNLAELPKRLLGSMKKGTPVIIQSE |
| | | domain- | Y2R2 | | AAECGLACMAMVASYHGHALDLPAM |
| | | containing | | | RRHFATSLKGMALRDLLEIGNGLHL |
| | | ABC | | | STRAVRLELEDLKQLKLPCILHWDH |
| | | transporter | | | NHFVVLTRVGSRGVTIHDPATGSRT |
| | | | | | LTMSEVSSHFTGVALEAWPTEFFAK |
| | | | | | KDERERIHVTEMIKRTAGIGRAALQ |
| | | | | | ILSISVLLELVTIAMPIGFQMIIDE |
| | | | | | VIVASDYDLLTLITIAFCFFLALQV |
| | | | | | LASFIRSWTSMLIGSGLVLQWKAGL |
| | | | | | FDHLMRLPLSYFEKRHVGDIVSRFG |
| | | | | | SVDSIQQTLTTTAITTLLDGGMSIA |
| | | | | | LIIMMWLYGGWLAGIAMIAVVLYTI |
| | | | | | LRVATYFRYRSLSEEAIVYAAKENT |
| | | | | | HEMESLRGMASLKVMNLKERRRSVW |
| | | | | | VNYLVDRINANLKVQKFDVVFQTAS |
| | | | | | KLIFGIDRIIIIYLGAKAILGDTLS |
| | | | | | VGMLIAFLSYKDQFAARMDSFVTTI |
| | | | | | LSLRMLSLHGERIADIALSEPEESQ |
| | | | | | IDAAEQAYDTAGTEKLGRLEIENVS |
| | | | | | FRYSDNEPDVLKNINITIEQNECVG |
| | | | | | ISGPSGTGKTTLMKIISGLMLPNEG |
| | | | | | EVRIDGKTLSTLGLNAYRDRIGCVL |
| | | | | | QDDRLFAGSIAENICSFDPNPDLRL |
| | | | | | MQQCAMLAAIHTEIAKMPMGYETFV |
| | | | | | GDMGSTLSGGQRQRIILARA |
| 4 | WP_009309353.1 | MULTISPECIES: | Klebsiella | 148 | LYRRPSLLLLDEATSNLDPENEAAI |
| | | peptidase | | | NAAIKQLSITRIVIAHRPSTLAMTD |
| | | domain- | | | RVIDLKELQDQASNAQMNNKLFETI |
| | | containing | | | IAKLNFSLIKKTPVILQSESAECGI |
| | | ABC | | | ASLAMICGHYGLDIDLFNFRQRFGS |
| | | transporter | | | PSQGASLLHLSKTAERAGLKNRALS |
| | | | | | LDLDEIKQLKLPCILHWGMNHYVVL |
| | | | | | TKVRKSSYVVHDPALGKRIIGLQEM |
| | | | | | SNNFTGVALELWPDQNFQQEKAKAR |
| | | | | | LRLLDLMRNIVGLKSALIKIFAFSV |
| | | | | | VIEAIGLLLPIGTQLVTDHVIMAHD |
| | | | | | QSLLSVICIGLVFFTLFRTFVSMLR |
| | | | | | AWTSLTLNTLTNIQWKTTLFDHLTS |
| | | | | | LPLSFFEKRHLGDIQSRFTSLDTIR |
| | | | | | STFTNSIVSGIIDSIMTIGLLIMLT |
| | | | | | LYGGWLVWVVVGFTLCYAIMRLATY |
| | | | | | RFYRRVAEEQVIKGARSSSHFMESL |
| | | | | | YGISTIKALNLKERRSQHWLNINID |
| | | | | | ACNAGIKQTRFDMMFSGINTFISSI |
| | | | | | DQVAILWLGAIMVIDNNMTLGMFMA |
| | | | | | FNSYRGQFSQRASNLIDLFMQLRML |
| | | | | | SLHNERLSEIVFSEPEKELPARRVF |
| | | | | | DENSGVKLEIKNLSYQYDPFSQPIF |
| | | | | | SNFNLQVEPGESIALVGPSGVGKTT |
| | | | | | LLKVMCGLLSPTSGEVIADNLDIYK |
| | | | | | IGLNNYRLGTACVLQEDRLFSGSIS |
| | | | | | DNISGFEDNADEELIVECARRSNIH |
| | | | | | DEIMKMPMGYETMIGELGLGISGGQ |
| | | | | | KQRLLIARALYRKPSILFMDEATSH |
| | | | | | LDLKNESAINQSIASLSITRIIVAH |
| | | | | | RPSTIASADRIIDLSQYGKQAS |
| 4 | WP_014837172.1 | MULTISPECIES: | Klebsiella | 149 | MNKELFKTITDKLNFSFRHKTPVIL |
| | | peptidase | | | QSEAAECGIACLAMVCGFYGLNIDL |
| | | domain- | | | FNFRHRYGSPSQGVTLMSLSKTAEH |
| | | containing | | | AGLKSRALSLDLDEIKQLKLPCVIH |
| | | ABC | | | WGMNHYVVLTKVRKSSYIVHDPALG |
| | | transporter | | | KRVIGIQEMSNNFTGVALELWPDQN |
| | | | | | FQQEQAKSRLRLLDLMRNIVGLKSV |
| | | | | | LLKIFAFSVVVEAIGLLLPIGTQLV |
| | | | | | TDHVIMAHDQSLLSVICIGLVFFTL |
| | | | | | FRTFISMLRAWISLTLNTLTNIQWK |
| | | | | | TTLFDHLTSLPLSFFEKRHLGDIQS |
| | | | | | RFSSLDTIRSTFTNSIVTGIIDSIM |
| | | | | | TVGLLIMLTLYGGWLVWVVVGFTLC |
| | | | | | YVIMRFATYSFYRRVAEEQVIKGAR |
| | | | | | SSSHFMESLYGISTIKALNLKERRS |
| | | | | | QHWLNINIDACNAGIKQTRFDMMFG |
| | | | | | GINTFITSIDQVAVLWLGAIMVIDN |
| | | | | | NMTLGMFMAFNAYRGQFSQRASSLI |
| | | | | | DLIMQLRMLSLHNERLSEIVFSEPE |
| | | | | | KELPSRRVFAENTGVKLEVKNLSYQ |
| | | | | | YDPFSQPIFSNLNITVEPGESIALV |
| | | | | | GPSGVGKTTLLKVMCGLLSPTSGDV |
| | | | | | IADNLDIQKIGLNNFRHGTACVLQE |
| | | | | | DRLFSGSLIDNISGFEDNADMDFIV |
| | | | | | ECARRCNIHDEIMKMPMGYETIVGE |
| | | | | | LGLGISGGQKQRLLIARALYRKPSI |
| | | | | | LFMDEATSHLDLKNESMINQSISGL |
| | | | | | SITRIIVAHRPSTIASADRIIDLSQ |
| | | | | | LRTLAPA |
| 4 | WP_016338368.1 | MULTISPECIES: | Klebsiella | 150 | MKSEVISLLKNKLDLGLKSRFPMIH |
| | | peptidase | | | QTESSECGLACLAMICGYYGKNIDI |
| | | domain- | | | FTLRQKFNLSSRGTDLASLNDIAVK |
| | | containing | | | MHMSTRAVSVELDELHSVKLPCILH |
| | | ABC | | | WDFNHFVVLTKIKNGKFTVHDPSVG |
| | | transporter | | | IVKVAKSELSKKFTGIVLEIWPGNS |
| | | | | | FLKENVKNKISLRAMLENTHGLYSA |
| | | | | | LTKIFFLSIIIESINLVMPVATQLV |
| | | | | | MDHAIPANDSGLLTLICFGLVFFVA |
| | | | | | LRTITGLIRSWTVLIMSSFINAQWQ |
| | | | | | KGLLQHLLKLPLEYFERRKIGDIQS |
| | | | | | RFGSLNTLQETFTTSVVGAIIDSIM |
| | | | | | IIGLLIMLVLYGGWLTWFVILFTCL |
| | | | | | YVFIRVLTYNRYRQLSEESLIKDAR |
| | | | | | ANSFFMETLYGIATVKVQGLNKRRE |
| | | | | | ENWFNLEVDSINTGIKVSRLNLFFG |
| | | | | | GINTLIATMEQILILWLGASLVISG |
| | | | | | NMTIGMFVAFSSYRGQFSDRIASFI |
| | | | | | DFLLRLQIMSLHNERVSDIALTEVT |
| | | | | | PFKDDIPVKNDMCPVSLSAKNISYK |
| | | | | | YDSYASYTLRHLNIEIQPGEHVAIT |
| | | | | | GASGTGKTTLLKVLCGLFPASEGNI |
| | | | | | FVDGIDIKELGVNNYQKLIACVLQD |
| | | | | | DRLFSGTLRDNICSFSTQIDDNWLI |
| | | | | | ECARFAQIHDDIERMPMGYDTLIGE |
| | | | | | LGEGLSGGQKQRIFIARAIYKKPSI |
| | | | | | LFMDEATSSLDHTNEQYINDAIKSL |
| | | | | | NITRIVIAHRKSTINSADRIIALQE |
| | | | | | NL |
| 4 | WP_023343012.1 | MULTISPECIES: | Klebsiella | 151 | MNNKLFETIIAKLNFSLIKKTPVIL |
| | | peptidase | | | QSESAECGIASLAMICGHYGLDIDL |
| | | domain- | | | FNFRQRFGSPSQGASLLHLSKTAER |
| | | containing | | | AGLKNRALSLDLDEIKQLKLPCILH |
| | | ABC | | | WGMNHYVVLTKVRKSSYVVHDPALG |
| | | transporter | | | KRIIGLQEMSNNFTGVALELWPDQN |
| | | | | | FQQEKAKARLRLLDLMRNIVGLKSA |
| | | | | | LIKIFAFSVIIEAIGLLLPIGTQLV |
| | | | | | TDHVIMAHDQSLLSVICIGLVFFTL |
| | | | | | FRTFVSMLRAWTSLTLNTLTNIQWK |
| | | | | | TTLFDHLTSLPLSFFEKRHLGDIQS |
| | | | | | RFTSLDTIRSTFTNSIVSGIIDSIM |
| | | | | | TIGLLIMLTLYGGWLVWVVVGFTLC |
| | | | | | YAIMRLATYRFYRRVAEEQVIKGAR |
| | | | | | SSSHFMESLYGISTIKALNLKERRS |
| | | | | | QHWLNINIDACNAGIKQTRFDMMFS |
| | | | | | GINTFISSIDQVAILWLGAIMVIDN |
| | | | | | NMTLGMFMAFNSYRGQFSQRASNLI |
| | | | | | DLFMQLRMLSLHNERLSEIVFSEPE |
| | | | | | KELPARRVFDENSGVKLEIKNLSYQ |
| | | | | | YDPFSQPIFSNFNLQVEPGESIALV |
| | | | | | GPSGVGKTTLLKVMCGLLSPTSGEV |
| | | | | | IADNLDIYKIGLNNYRLGTACVLQE |
| | | | | | DRLFSGSISDNISGFEDNADEELIV |
| | | | | | ECARRSNIHDEIMKMPMGYETMIGE |
| | | | | | LGLGISGGQKQRLLIARALYRKPSI |
| | | | | | LFMDEATSHLDLKNESAINQSIASL |
| | | | | | SITRIIVAHRPSTIASADRIIDLSQ |
| | | | | | YGKQAS |
| 4 | WP_049086514.1 | MULTISPECIES: | Klebsiella | 152 | MNKNIFKTITDSLNFSLRKKIPVIL |
| | | peptidase | | | QSESAECGIACITMISCYYGLNIDL |
| | | domain- | | | FNFRKRYGSPSQGFTLLSLSNNAER |
| | | containing | | | AGLKSRALSLDLDEINQLKLPCIIH |
| | | ABC | | | WGMNHYVVLTKVRRSSFIIHDPALG |
| | | transporter | | | KRVIGLQEMSNNFTGIALELWPDQN |
| | | | | | FQQEKAKSRLRLLDLMRNIVGLKST |
| | | | | | LIKIFAYSVVIEAIGLLIPIGTQMV |
| | | | | | TDHVIMAHDQSLLAVICIGLVSFTL |
| | | | | | FRTFISMLRAWTSLTLNTLTNIQWK |
| | | | | | TTLFDHLTSLPLSFFEKRHLGDIQS |
| | | | | | RFSSLDIIRSTFTNSIVTGIIDTIM |
| | | | | | TIGLLIMLTLYGGWLVWVVVGFTLC |
| | | | | | YAIMRLATYRFYRRVSEEQVIKGAR |
| | | | | | SSSHFMESLYGIATIKALNLKERRS |
| | | | | | QHWLNLNIDACNAGIKQTRFDMMFS |
| | | | | | GINTFISSIDQVAVLWLGALAVINN |
| | | | | | DMTLGMFMAFNAYRGQFSQRASSLI |
| | | | | | DLFMQLRMLSLHNERLSEIAFSEPE |
| | | | | | KDMPTRRIFDKDEGVKLEVKNLSYQ |
| | | | | | YDPFSQPIFSNLNIIIEQGESVALI |
| | | | | | GPSGIGKTTLLKVMCGLLSPSAGDV |
| | | | | | LVDNLNISKIGLNNYRLSTACVLQE |
| | | | | | DRLFSGSILENISGFEDNVDEEFVI |
| | | | | | ECARHSNIHDEIMKMPMGYETLIGE |
| | | | | | LGSGISGGQKQRLLIARALYRKPCI |
| | | | | | LFMDEATSHLDLNNEASINNAISKL |
| | | | | | SITRIIVAHRPSTIASADRVIDLSE |
| | | | | | SASK |
| 4 | WP_080897633.1 | MULTISPECIES: | Klebsiella | 153 | MNKKLFETIIAKLNFSLIKKTPVIL |
| | | peptidase | | | QSESAECGIASLAMICGHYGLDIDL |
| | | domain- | | | FNFRQRFGSPSQGASLLHLSKTAER |
| | | containing | | | AGLKNRALSLDLDEIKQLKLPCILH |
| | | ABC | | | WGMNHYVVLTKVRKSSYVVHDPALG |
| | | transporter | | | KRIIGLQEMSNNFTGVALELWPDQN |
| | | | | | FQQEKAKARLRLLDLMRNIVGLKSA |
| | | | | | LIKIFAFSVVIEAIGLLLPIGTQLV |
| | | | | | TDHVIMAHDQSLLSVICIGLVFFTL |
| | | | | | FRTFVSMLRAWTSLTLNTLTNIQWK |
| | | | | | TTLFDHLTSLPLSFFEKRHLGDIQS |
| | | | | | RFTSLDTIRSTFTNSIVSGIIDSIM |
| | | | | | TIGLLIMLTLYGGWLVWVVVGFTLC |
| | | | | | YAIMRLATYRFYRRVAEEQVIKGAR |
| | | | | | SSSHFMESLYGISTIKALNLKERRS |
| | | | | | QHWLNINIDACNAGIKQTRFDMMFS |
| | | | | | GINTFISSIDQVAILWLGAIMVIDN |
| | | | | | NMTLGMFMAFNSYRGQFSQRASNLI |
| | | | | | DLFMQLRMLSLHNERLSEIVFSEPE |
| | | | | | KELPARRVFDENSGVKLEIKNLSYQ |
| | | | | | YDPFSQPIFSNFNLQVEPGESIALV |
| | | | | | GPSGVGKTTLLKVMCGLLSPTSGEV |
| | | | | | IADNLDIYKIGLNNYRLGTACVLQE |
| | | | | | DRLFSGSISDNISGFEDNADEELIV |
| | | | | | ECARRSNIHDEIMKMPMGYETMIGE |
| | | | | | LGLGISGGQKQRLLIARALYRKPSI |
| | | | | | LFMDEATSHLDLKNEAAINQSIASL |
| | | | | | SITRVIVAHRPSTIASADRIIDLSQ |
| | | | | | YGKQAS |
| 4 | WP_015368662.1 | peptidase | Klebsiella | 154 | MTNELFEKITAKVNFSILKKTPVIL |
| | | domain- | aerogenes | | QSEAAECGIACLAMVCGHYGLDIDL |
| | | containing | | | FNFRQRFGSPSQGVTLMSLSKTAEH |
| | | ABC | | | AGLKSRALSLDLDEIRQLKLPCVIH |
| | | transporter | | | WGMNHYVVLTKVRKSSFVVHDPALG |
| | | | | | KRIIGIQEMSNNFTGIALELWPDQN |
| | | | | | FQQEKAKSRLRLLDLMRNIVGLKSA |
| | | | | | LLKIFAFSVVVEAIGLLLPIGTQLV |
| | | | | | TDHVIMAHDQSLLSVICIGLVFFTL |
| | | | | | FRTFISMLRAWTSLTLNTLTNIQWK |
| | | | | | TTLFDHLASLPLAFFEKRHLGDIQS |
| | | | | | RFSSLDTIRSTFTNSIVSGIIDSIM |
| | | | | | TIGLLIMLTLYGGWLTWVVVGFTLC |
| | | | | | YALMRLGTYRFYRRVAEEQVIKGAR |
| | | | | | SSSHFMESLYGISTIKALNLKERRS |
| | | | | | QHWLNINIDACNAGIKQTRFDMMFS |
| | | | | | GINTFITAIDQVAVLWLGAIMVIDN |
| | | | | | EMTLGMFMAFNAYRGQFSQRASSLV |
| | | | | | DLTMQLRMLSLHNERLSEIVFSEPE |
| | | | | | QELPSREVFSKDAGARLEVRNLSYQ |
| | | | | | YDPFSQPIFSNLNITVEPGESVALV |
| | | | | | GPSGVGKTTLLKVMCGLLSPTSGDV |
| | | | | | FADNLDIQKIGLNNYRHGTACVLQE |
| | | | | | DRLFSGSLIDNISGFEDNADVDFVV |
| | | | | | ECAKRCNIHDEIMKMPMGYETIVGE |
| | | | | | LGLGISGGQKQRILIARALYRKPSI |
| | | | | | LFMDEATSHLDLRNESVINQSISGL |
| | | | | | SITRVIVAHRPSTIASADRVIDLSQ |
| | | | | | SRIPMPA |
| 4 | WP_015705588.1 | peptidase | Klebsiella | 155 | MNRNHFISSIEEKLDLKFRRRVPVV |
| | | domain- | aerogenes | | HQTETSECGLACLAMICGHYGRNID |
| | | containing | | | MIALRRQINLSARGASLASVQQTGI |
| | | ABC | | | ELGMESRALSLDLHELNALRMPCIL |
| | | transporter | | | HWNFNHFVVLVSVRKNRFVLHDPAR |
| | | | | | GRRTVGSDEMSQCFTGVALEVWPGS |
| | | | | | TFVQETMKSRIGIRALISSVQGIRG |
| | | | | | VLSKIFCLSLIIEAIGLVMPVGTQL |
| | | | | | VMDHAIPANDRGLLMLICGVLMFFI |
| | | | | | LGRTLVSVIRAWSSLAMESLINVQW |
| | | | | | QSGLFRHLLQLPLTYFERRKMGDIH |
| | | | | | SRFSSLDALRATFTTCVVGAMMDSI |
| | | | | | MVAGLLLMLILYGGKLTVFVLGFTA |
| | | | | | VYVFIRILTYSRYRQLSEEALIAGA |
| | | | | | RAGSYFMETLYGIATVKMQGMAERR |
| | | | | | GSHWLNLKIDAINTGIRLSRLDMFF |
| | | | | | GGINTFVATCDQVTILWLGISLVME |
| | | | | | NQMTIGMFVAFGAFRAQFSDRVGSL |
| | | | | | VNFLLQLRMMSLHNERISDIALNEK |
| | | | | | EERKPDLGLKATMSPASLEIKALSF |
| | | | | | RYDSQSAPVFSGLSFRIRAGESVAI |
| | | | | | TGPSGSGKTTLLKVLCGLSEPISGT |
| | | | | | VLMNGTDIRQLGINNYHRLIACVMQ |
| | | | | | DDRLFSGSIRENICGFTENIDDAWM |
| | | | | | RECARTSRIHDVILALPMGYETLIG |
| | | | | | ELGEGLSGGQKQRIFIARALYRRPD |
| | | | | | ILFMDEATSALDAECEKDVSLAMKK |
| | | | | | LNITRIIIAHRETTLQTADRIITLG |
| | | | | | ESN |
| 4 | WP_112216234.1 | peptidase | Klebsiella | 156 | MTSELFNTIISRLNFSLCKKTPVII |
| | | domain- | huaxiensis | | QSEASECGIACLSMVCGYYGLEIDL |
| | | containing | | | FNFRQRFGSSSQGVTLLELSKVAEQ |
| | | ABC | | | ANLKNRALTLDLDEIKQLKLPCILH |
| | | transporter | | | WGMNHYVVLVAVKRSSFIVHDPALG |
| | | | | | KRVIGIQEMSNNFTGVAMELWPDNN |
| | | | | | FRQEKVKSRLRLMDLMKNVVGLKSA |
| | | | | | LTKIFALSVVIEAINLLMPIGTQLV |
| | | | | | TDHVIIAHDHSLLSVICIGLVVFTL |
| | | | | | FRTFISMLRAWTSLTLNTLTDIQWK |
| | | | | | TTLFDHLFSLPLTFFEKRHLGDIQS |
| | | | | | RFSSLDVIRSTFTNSIVTSIIDIIM |
| | | | | | TVGLLIMMSLYGGWLVWVVIGFTLC |
| | | | | | YAVMRFGTYRFYRRIAEERVIKNAR |
| | | | | | SGSHFMESLYGISTIKALNLKERRS |
| | | | | | KNWLNINVEACNAGIKQTRFDMMFG |
| | | | | | GINTFISSMDQVVILWLGAVMVIEN |
| | | | | | NMTLGMFMAFNAYRGQFSQRASSLI |
| | | | | | DLVMQLRMLSLHNERLSEIVFSEPE |
| | | | | | DEQPSRRIFEINKGVSLAVKDLSYQ |
| | | | | | YDPFSRPIFSELNLTINPGESVALV |
| | | | | | GPSGIGKTTLLKVMCGLLVPSNGQI |
| | | | | | LADDLDVSKIGLNNYRLSTACVLQD |
| | | | | | DRLFSGSIADNICSFEDNPDQERII |
| | | | | | ACAQYCNIHEEIMRMSMGYESIVGE |
| | | | | | LGLGISGGQKQRILIARALYRQPSI |
| | | | | | LFMDEATSHLDLANESFINQSISNL |
| | | | | | KITRIIVAHRPSTIASADRVIDLSQ |
| | | | | | YFKNASQTASLI |
| 4 | WP_004178319.1 | peptidase | Klebsiella | 157 | MTNEIFTTIISRLNFSLRHKTPVIL |
| | | domain- | pneumoniae | | QSEATECGIACLAMVCGHYGLDIDL |
| | | containing | | | FNFRQRFGSASQGVTMMSLSKTADH |
| | | ABC | | | AGLKSRALSLDLNEIRQLKLPCILH |
| | | transporter | | | WGMNHYVVLTKVRKSSFIVHDPALG |
| | | | | | KRVIGNQEMSNYFTGIALELWPDQN |
| | | | | | FQQEKAKSRLRLLDLMRNIIGLKST |
| | | | | | LLKIFAFSVVVEAIGLLLPIGTQLV |
| | | | | | TDHVIMAHDQSLLSVICIGLVFFTL |
| | | | | | FRTFISMLRAWTSLTLNTLTNIQWK |
| | | | | | TTLFDHLASLPLSFFEKRHLGDIQS |
| | | | | | RFSSLDTIRSTFTNSIVSGIIDSIM |
| | | | | | TIGLLIMLTLYGGWLTWVVVGFTLC |
| | | | | | YAIMRFATYRFYRRVAEEQVIKGAR |
| | | | | | SSSHFMESLYGISTIKALNLKERRS |
| | | | | | QHWLNINIEACNAGIKQTRFDMMFG |
| | | | | | GINTFITTIDQVAVLWLGAIMVIDN |
| | | | | | NMTLGMFMAFNAYRGQFSQRASSLI |
| | | | | | DLCMQLRMLSLHNERLSEIVFSEPE |
| | | | | | QELPAREVFSADSGAKLEVKNLCYQ |
| | | | | | YDPFSQPIFSNLNITVEPGESVALI |
| | | | | | GPSGVGKTTLLKVMCGLLSPTSGDV |
| | | | | | LADNLDITKIGLNNYRHGTACVLQE |
| | | | | | DRLFSGSLIDNISGFEDNADINFVM |
| | | | | | ECARRCNIHDEIMKMPMGYETIVGE |
| | | | | | LGLGISGGQKQRILIARALYRKPSI |
| | | | | | LFMDEATSHLDLKNESVINQSISAL |
| | | | | | SITRIIVAHRPSTIASADRVIDLSQ |
| | | | | | TKNLAPA |
| 4 | WP_004192519.1 | peptidase | Klebsiella | 158 | MTNELFKTVVAKLNFSLVNKTPVIL |
| | | domain- | pneumoniae | | QSESSECGIACLAMVCGFYGLNIDL |
| | | containing | | | FNFRQRFGSPSQGSSLMSLSKTAEH |
| | | ABC | | | AGLKSRALALDLDEIKQLKLPCIIH |
| | | transporter | | | WGMNHYVVLTKVRKNAFVIHDPALG |
| | | | | | KRIIGINEMSNNFTGVALELWPDHN |
| | | | | | FQQEEAKSRLRLLDLMHKIVGLKSA |
| | | | | | LIKIFAYSVVVEAIGLLLPIGTQLV |
| | | | | | TDHVIMAHDQSLLSVICIGLIFFTL |
| | | | | | FRTFISMLRAWTSLTLNTLTNIQWK |
| | | | | | TTLFDHLTSLPLSFFEKRHLGDIQS |
| | | | | | RFSSLDTIRSIFTNSIVTGIIDSIM |
| | | | | | TIGLLVMLTLYGGWLVWVVIGFTIC |
| | | | | | YAIMRFATYSFYRRVTEEQVVKGAR |
| | | | | | ANSHFMESLYGISTIKALNLKERRS |
| | | | | | QHWLNLNVDASNAGIKQTRFDMMFG |
| | | | | | GINTFITSIDQVVVLWLGAIMVIDN |
| | | | | | EMTLGMFMAFNAYRGQFSQRASNLI |
| | | | | | DLVMQLRMLSLHNERLSEIAFSEPE |
| | | | | | KELPSRRVFTENTGVKLEVRNLSYQ |
| | | | | | YDPFSQPIFTNLNITVAPGESVALI |
| | | | | | GPSGVGKTTLLKVMCGLLSPDSGEI |
| | | | | | LADNLEIKKIGLNNYRLGIACVLQE |
| | | | | | DRLFSGSLIDNISGFDDSADLDFVM |
| | | | | | ECARRCNIHDEIMKMAMGYETIVGE |
| | | | | | LGLGISGGQKQRILIARALYRKPSI |
| | | | | | LFMDEATSHLDLRNESVINQSISAL |
| | | | | | SITRIIVAHRPSTIASADRVIDLSQ |
| | | | | | SKSVTTA |
| 4 | WP_016831378.1 | peptidase | Klebsiella | 159 | MTNELFKTVVAKLNFSLVNKTPVIL |
| | | domain- | pneumoniae | | QSESSECGIACLAMVCGFYGLNIDL |
| | | containing | | | FNFRQRFGSPSQGSSLMSLSKTAKH |
| | | ABC | | | AGLKSRALALDLDEIKQLKLPCIIH |
| | | transporter | | | WGMNHYVVLTKVRKNGFVIHDPALG |
| | | | | | KRIIGINEMSNNFTGVALELWPDHN |
| | | | | | FQQEEAKSRLRLLDLMHKIVGLKSA |
| | | | | | LIKIFAYSVVVEAIGLLLPIGTQLV |
| | | | | | TDHVIMAHDQSLLSVICIGLIFFTL |
| | | | | | FRTFISMLRAWTSLTLNTLTNIQWK |
| | | | | | TTLFDHLTSLPLSFFEKRHLGDIQS |
| | | | | | RFSSLDTIRSIFTNSIVTGIIDSIM |
| | | | | | TIGLLVMLTLYGGWLVWVVIGFTIC |
| | | | | | YAIMRFATYSFYRRVTEEQVVKGAR |
| | | | | | ANSHFMESLYGISTIKALNLKERRS |
| | | | | | QHWLNLNVDASNAGIKQTRFDMMFG |
| | | | | | GINTFITSIDQVVVLWLGAIMVIDN |
| | | | | | EMTLGMFMAFNAYRGQFSQRASNLI |
| | | | | | DLVMQLRMLSLHNERLSEIAFSEPE |
| | | | | | KELPSRRVFTENTGVKLEVRNLSYQ |
| | | | | | YDPFSQPIFTNLNITVAPGESVALI |
| | | | | | GPSGVGKTTLLKVMCGLLSPDSGEI |
| | | | | | LADNLEIKKIGLNNYRLGIACVLQE |
| | | | | | DRLFSGSLIDNISGFDDSADLDFVM |
| | | | | | ECARRCNIHDEIMKMAMGYETIVGE |
| | | | | | LGLGISGGQKQRILIARALYRKPSI |
| | | | | | LFMDEATSHLDLRNESVINQSISAL |
| | | | | | SITRIIVAHRPSTIASADRVIDLSQ |
| | | | | | SKSVTTA |
| 4 | WP_023302370.1 | peptidase | Klebsiella | 160 | MNNKLFETIIAKLNFSLIKKTPVIL |
| | | domain- | pneumoniae | | QSESAECGIASLAMICGHYGLDIDL |
| | | containing | | | FNFRQRFGSPSQGASLLHLSKTAER |
| | | ABC | | | AGLKNRALSLDLDEIKQLKLPCILH |
| | | transporter | | | WGMNHYVVLTKVRKSSYVVHDPALG |
| | | | | | KRIIGLQEMSNNFTGVALELWPDQN |
| | | | | | FQQEKAKARLRLLDLMRNIVGLKSA |
| | | | | | LIKIFAFSVIIEAIGLLLPIGTQLV |
| | | | | | TDHVIMAHDQSLLSVICIGLVFFTL |
| | | | | | FRTFVSMLRAWTSLTLNTLTNIQWK |
| | | | | | TTLFDHLTSLPLSFFEKRHLGDIQS |
| | | | | | RFTSLDTIRSTFTNSIVSGIIDSIM |
| | | | | | TIGLLIMLTLYGGWLVWVVVGFTLC |
| | | | | | YAIMRLATYRFYRRVAEEQVIKGAR |
| | | | | | SSSHFMESLYGISTIKALNLKERRS |
| | | | | | QHWLNINIDACNAGIKQTRFDMMFS |
| | | | | | GINTFISSIDQVAILWLGAIMVIDN |
| | | | | | NMTLGMFMAFNSYRGQFSQRASNLI |
| | | | | | DLFMQLRMLSLHNERLSEIVFSEPE |
| | | | | | KELPERRVFDENSGVKLEIKNLSYQ |
| | | | | | YDPFSQPIFSNFNLQVEPGESIALV |
| | | | | | GPSGVGKTTLLKVMCGLLSPTSGEV |
| | | | | | IADNLDIYKIGLNNYRLGTACVLQE |
| | | | | | DRLFSGSISDNISGFEDNADEELIV |
| | | | | | ECARRSNIHDEIMKMPMGYETMIGE |
| | | | | | LGLGISGGQKQRLLIARALYRKPSI |
| | | | | | LFMDEATSHLDLKNESAINQSIASL |
| | | | | | SITRIIVAHRPSTIASADRIIDLSQ |
| | | | | | YGKQAS |
| 4 | WP_032446579.1 | peptidase | Klebsiella | 161 | |
| | | domain- | pneumoniae | | |
| | | containing | | | |
| | | ABC | | | |
| | | transporter | | | |
| 4 | WP_107320695.1 | peptidase | Klebsiella | 162 | |
| | | domain- | pneumoniae | | |
| | | containing | | | |
| | | ABC | | | |
| | | transporter | | | |
| 4 | YP_005224683.1 | microcin | Klebsiella | 163 | MTNELFEKIIAKVNFSLLKKTPVIL |
| | | H47 ABC | pneumoniae | | QSEAAECGIACLAMVCGHYGLDIDL |
| | | transporter | subsp. | | FNFRQRFGSPSQGVTLMSLSKTAEH |
| | | permease/A | pneumoniae | | AGLKSRALSLDLDEIRQLKLPCVLH |
| | | TP-binding | HS11286 | | WGMNHYVVLTKVRKSSFIVHDPALG |
| | | protein | | | KRIIGIQEMSNYFTGVALELWPDQN |
| | | | | | FQQEKAKSRLRLLDLMRNIVGLKSA |
| | | | | | LLKIFAFSVVVEAIGLLLPIGTQLV |
| | | | | | TDHVIMAHDQSLLSVICIGLVFFTL |
| | | | | | FRTFISMLRAWTSLTLNTLTSIQWK |
| | | | | | TTLFDHLASLPLSFFEKRHLGDIQS |
| | | | | | RFSSLDTIRSTFTNSIVSGIIDSIM |
| | | | | | TIGLLIMLTLYGGWLTWVVVGFTLC |
| | | | | | YAIMRFATYRFYRRVAEEQVIKGAR |
| | | | | | SSSHFMESLYGISTIKALNLKERRS |
| | | | | | QHWLNINIEACNAGIKQTRFDMLFS |
| | | | | | GINTFITAIDQVAVLWLGAIMVIDN |
| | | | | | NMTLGMFMAFNAYRGQFSQRASSLV |
| | | | | | DLCMQLRMLSLHNERLSEIVFSEPE |
| | | | | | QELPTREVFSADSGAKLEVKNLCYQ |
| | | | | | YDPFSQPIFSNLNITVEPGESVALV |
| | | | | | GPSGVGKTTLLKVMCGLLSPTSGDV |
| | | | | | LADNLDITKIGLNNYRLGIACVLQE |
| | | | | | DRLFSGSLIDNISGFEDNADIDFVM |
| | | | | | ECAKRCNIHDEIIKMPMGYETIVGE |
| | | | | | LGLGISGGQKQRILIARALYRRPSI |
| | | | | | LFMDEATSHLDLKNESVINQSISAL |
| | | | | | SITRIIVAHRPSTIASADRVIDLSQ |
| | | | | | TKTLSPA |
| 4 | WP_064562844.1 | peptidase | Kosakonia | 164 | MPVLLQTETAECGLVSLAMIADWYN |
| | | domain- | oryzae | | LPTDVRNLRQYGGFSIRGATLRMVM |
| | | containing | | | DTAASIGMKSRALQLDMDNLPALKL |
| | | ABC | | | PCILHWDLNHFVVLAAIRRGKVVIH |
| | | transporter | | | DPAAGRRIMGMKEVSLHFTGVALEL |
| | | | | | WPAGEMKQEQQPRNRLRLSTLTGRI |
| | | | | | AGFAITLTKLFCYSILVESCGLLLP |
| | | | | | AGMQLIMDHAIPANDTSLLTLICLG |
| | | | | | LLFFVLFRTGISMLRGWTSLVASTL |
| | | | | | IDIQWKAGLFERMLSLPLAYFEKRR |
| | | | | | LGDIQSRFSSLGILRTTLTNNVVSA |
| | | | | | IMDGIMLVGLIVMMLLYGGWLLWIV |
| | | | | | LAFFLAWGGFRLMTYPAWRRASEEL |
| | | | | | IVREARAGSHFMETLYSINTIKALG |
| | | | | | LDKQRAQHWINLNVESANATVRKTR |
| | | | | | YEMLFSGGNILIAALEQIALLWLGA |
| | | | | | HQVIDGQLTLGMFVAFNTYRGQFAD |
| | | | | | RANNLLNMLLQLRMLSLHKERVADI |
| | | | | | AHAEAESPLSEHTRQLLAPGEPATL |
| | | | | | TLHNLSFQYDPFVPPLFSELHIEIN |
| | | | | | AGESVAIIGPSGRGKTTLLKIMAGL |
| | | | | | LQPTTGEVHINGMNIRTTGLRNYRA |
| | | | | | CIACVLQDDSLLAGSVADNISAFDP |
| | | | | | QPDEKWMQHCAQLSNIHEDILRMPM |
| | | | | | GYQTLISELGGSLSGGQKQRLLIAR |
| | | | | | ALYRRPSILFLDEATSHLDTVNEAQ |
| | | | | | INTAIKSLRITRVIIAHRPSTIASA |
| | | | | | DRVIDLDRGK |
| 5 | PDI_PCAT_mcpB | peptidase | Escherichia coli | 165 | MESINWKVRKQLPVIRQTESAECGL |
| | | domain- | | | ACLAMIACWHGLKTDLSTLRERFNI |
| | | containing | | | GIQGMTLQRLIECAASIHLSSRAVR |
| | | ABC | | | LEPEDLRCLNLPSILHWDMNHFVVL |
| | | transporter | | | HKVRGNRLYIHDPDRGKITISLLDA |
| | | | | | GKHFTGVALELTPASDFTPRNERKK |
| | | | | | IHLRQLTGKTPGLLASMTKIIIFAL |
| | | | | | ALEILALGGPLLNQLVIDEVLVAAD |
| | | | | | RSLLYVIIVALLLLSLIQLLLSLAR |
| | | | | | QWATISLSVNFNMQWTARVFHHLVR |
| | | | | | LPLAWFDARSKGSINARFEAVDIIQ |
| | | | | | QALTTQVLEGILDMLLIVTALCMML |
| | | | | | LYSPGMTLIAVIAAIIYGALRALWY |
| | | | | | PALRQSVEDVWDAGTKESGHFLETL |
| | | | | | NGIQSLRINGVTIHREAAWLNLNVT |
| | | | | | RRNTQLRQNRLQMSYELTHTLTESV |
| | | | | | VSAIILWQGAVEVLDGTFTVGMLVA |
| | | | | | YLSYQMRFSSSISNLTDNFFSWRML |
| | | | | | DVYNERLADIVLTPQEGHQNQHHWA |
| | | | | | NHNETISASQYREHKYDNTHPPLLI |
| | | | | | EKITFSHKGADKPILDNASLMLFPG |
| | | | | | EILAITGKSGCGKSTLVKLILGIHT |
| | | | | | PSEGRINAFGIPHTHSDYFQVRQRI |
| | | | | | GTVLQDDYLFKGSIADNIMFFSEIR |
| | | | | | DHEHMRKCASLALIDSDIMAMPMGY |
| | | | | | QTLLGETGGGLSGGQKQRILLARAL |
| | | | | | YKKPGLLLLDEATSHLDVESEIEIS |
| | | | | | QTLRQLGIPVLLIAHRPETIASADR |
| | | | | | VLYLRDGHFSEITYRPARTHNINNH |
| | | | | | PNRR |
| 5 | WP_000443943.1 | MULTISPECIES: | Enterobacteriaceae | 166 | MESINWKVRKQLPVIRQTESAECGL |
| | | peptidase | | | ACLAMIACWHGLKTDLSTLRERFNI |
| | | domain- | | | GIQGMTLQRLIECAASIHLSSRAVR |
| | | containing | | | LEPEDLRCLNLPSILHWDMNHFVVL |
| | | ABC | | | HKVRGNRLYIHDPDRGKITISLLDA |
| | | transporter | | | GKHFTGVALELTPASDFTPRNERKK |
| | | | | | IHLRQLTGKTPGLLASMTKIIIFAL |
| | | | | | ALEILALGGPLLNQLVIDEVLVAAD |
| | | | | | RSLLYVIIVALLLLSLIQLLLSLAR |
| | | | | | QWATISLSVNFNMQWTARVFHHLVR |
| | | | | | LPLAWFDARSKGSINARFEAVDIIQ |
| | | | | | QALTTQVLEGILDMLLIVTALCMML |
| | | | | | LYSPGMTLIAVIAAIIYGALRALWY |
| | | | | | PALRQSVEDVWDAGTKESGHFLETL |
| | | | | | NGIQSLRINGVTIHREAAWLNLNVT |
| | | | | | RRNTQLRQNRLQMSYELTHTLTESV |
| | | | | | VSAIILWQGAVEVLDGTFTVGMLVA |
| | | | | | YLSYQMRFSSSISNLTDNFFSWRML |
| | | | | | DVYNERLADIVLTPQEGHQNQHHWA |
| | | | | | NHNETISASQYREHKYDNTHPPLLI |
| | | | | | EKITFSHKGADKPILDNASLMLFPG |
| | | | | | EILAITGKSGCGKSTLVKLILGIHT |
| | | | | | PSEGRINAFGIPHTHSDYFQVRQRI |
| | | | | | GTVLQDDYLFKGSIADNIMFFSEIR |
| | | | | | DHEHMRKCASLALIDSDIMAMPMGY |
| | | | | | QTLLGETGGGLSGGQKQRILLARAL |
| | | | | | YKKPGLLLLDEATSHLDVESEIEIS |
| | | | | | QTLRQLGIPVLLIAHRPETIASADR |
| | | | | | VLYLRDGHFSEITYRPARTHNINNH |
| | | | | | PNRR |
| 5 | WP_061330275.1 | MULTISPECIES: | Enterobacteriaceae | 167 | MESINWKVRKQLPVIRQTESAECGL |
| | | peptidase | | | ACLAMIACWHGLKTDLSTLRERFNI |
| | | domain- | | | GIQGMTLQRLIECAASIHLSSRAVR |
| | | containing | | | LEPEDLRCLNLPSILHWDMNHFVVL |
| | | ABC | | | HKVRGNRLYIHDPDRGKITISLLDA |
| | | transporter | | | GKHFTGVALELTPASDFTPRNERKK |
| | | | | | IHLRQLTGKTPGLLASMTKIIIFAL |
| | | | | | ALEILALGGPLLNQLVIDEVLVAAD |
| | | | | | RSLLYVIIVALLLLSLIQLLLSLAR |
| | | | | | QWATISLSVNFNMQWTARVFHHLVR |
| | | | | | LPLAWFDARSKGSINARFEAVDIIQ |
| | | | | | QALTTQVLEGILDMLLIVTALCMML |
| | | | | | LYSPGMTLIAVIAAIIYGALRALWY |
| | | | | | PALRQSVEDVWDAGTKESGHFLETL |
| | | | | | NGIQSLRINGVTIHREAAWLNLNVT |
| | | | | | RRNTQLRQNRLQMSYELTHTLTESV |
| | | | | | VSAIILWQGAVEVLDGTFTVGMLVA |
| | | | | | YLSYQMRFSSSISNLTDNFFSWRML |
| | | | | | DVYNERLADIVLTPQEGHQNQHHWA |
| | | | | | NHNETISASQYREHKYDNTHPPLLI |
| | | | | | EKITFSHKGADKPILDNASLMLFPG |
| | | | | | EILAITGKSGCGKSTLVKLILGIHT |
| | | | | | PSEGRINAFGIPHTHSDYFQVRQRI |
| | | | | | GTVLQDDYLFKGSIADNIMFFSEIR |
| | | | | | DHEHMRKCASLALIDSDIMAMPMGY |
| | | | | | QTLLGETGGGLSGGQKQRILLARAL |
| | | | | | YKKPGLLLLDEATSHLDVESEIEIS |
| | | | | | QTLRQLGIPVLLIAHRPETIASADR |
| | | | | | VLYLRDGHFSEIKYRPVRTHNINNH |
| | | | | | PNRR |
| 5 | WP_061348417.1 | MULTISPECIES: | Escherichia | 168 | MESINWKVRKQLPVIRQTESAECGL |
| | | peptidase | | | ACLAMIACWHGLKTDLSTLRERFNI |
| | | domain- | | | GIQGMTLQRLIECAASIHLSSRAVR |
| | | containing | | | LEPEDLRCLNLPSILHWDMNHFVVL |
| | | ABC | | | HKVRGNRLYIHDPDRGKITISLLDA |
| | | transporter | | | GKHFTGVALELTPASDFTPRNERKK |
| | | | | | IHLRQLTGKTPGLLASMTKIIIFAL |
| | | | | | ALEILALGGPLLNQLVIDEVLVAAD |
| | | | | | RSLLYVIIVALLLLSLIQLLLSLAR |
| | | | | | QWATISLSVNFNMQWTARVFHHLVR |
| | | | | | LPLAWFDARSKGSINARFEAVDIIQ |
| | | | | | QALTTQVLEGILDMLLIVTALCMML |
| | | | | | LYSPGMTLIAVIAAIIYGALRALWY |
| | | | | | PALRQSVEDVWDAGTKESGHFLETL |
| | | | | | NGIQSLRINGVTIHREAAWLNLNVT |
| | | | | | RRNTQLRQNRLQMSYELTHTLTESV |
| | | | | | VSAIILWQGAVEVLDGTFTVGMLVA |
| | | | | | YLSYQMRFSSSISNLTDNFFSWRML |
| | | | | | DVYNERLADIVLTPQEGHQNQHHWA |
| | | | | | NHNETISASQYREHKYDNTHPPLLI |
| | | | | | EKITFSHKGADKPILDNASLMLFPG |
| | | | | | EILAITGKSGCGKSTLVKLILGIHT |
| | | | | | PSEGRINAFGIPHTHSDYFQVRQRI |
| | | | | | GTVLQDDYLFKGSIADNIMFFSEIR |
| | | | | | DHEHMRKCASLALIDSDIMAMPMGY |
| | | | | | QTLLGETGGGLSGGQKQRILLARAL |
| | | | | | YKKPSLLLLDEATSHLDVESEIEIS |
| | | | | | QTLRQLGIPVLLIAHRPETIASADR |
| | | | | | VLYLRDGHFSEIKYRPVRTHNINNH |
| | | | | | PNRR |
| 6 | S_PCAT_mcsB | peptidase | Escherichia | 169 | MDTLKWTGRKQLPLIRQTESAECGL |
| | | domain- | coli | | ACLVMMACWHGLQTDLPTLRERFSI |
| | | containing | | | STQGMTLQRLIECAAGIRLSSRAVR |
| | | ABC | | | LEPEDLKSLSLPCILHWNMNHFVVL |
| | | transporter | | | YKVRGSRLVIHDPDKGKITLSLQDA |
| | | | | | GKHFTGVALELTPASDFTARDERKK |
| | | | | | IRLRQLTGRTPGLLAAMSRIIIFAL |
| | | | | | ALEILTLSSPLLNQLVIDEVLVAAD |
| | | | | | RSLLTVIIIALLLLSMTQMLLSLAR |
| | | | | | QWASITLSVNFNMQWTARVFHHLVR |
| | | | | | LPLGWFDARSKGSINARFEAVDTIQ |
| | | | | | EALTTQVLEGILDVLLVITALCMML |
| | | | | | LYSPEMTLIAVLAAIIYGVLRALWY |
| | | | | | PSLRQSAEDAWDAGARESGHFLETL |
| | | | | | NGILSLRINGVTAHREAAWLNLNIV |
| | | | | | RRNTQLRQSRQLMCYDIAHTLTGSV |
| | | | | | VSAIILWKGAGEVLNGTFTVGMLVA |
| | | | | | YLSYQMRFSSSISSLTDKYFSWRML |
| | | | | | DIYNERLADIVLTPTEDYQQQPAQE |
| | | | | | DNDTSVFPSVFRGDVADGARVPLSL |
| | | | | | EHITFSHKGGNKPILRGVSLTLHPG |
| | | | | | EVMAITGQSGCGKSTLVKLILGIHI |
| | | | | | PDEGTIRAFGIPHTHPDYFQVRQRI |
| | | | | | GTVLQDDHLFRGSIADNIIFFSGDR |
| | | | | | NHERMIQCARLALIDSDIMSMPMGY |
| | | | | | QTLIGETGGGLSGGQKQRILLARAL |
| | | | | | YKKPGFLLLDEATSHLDVESEIQIS |
| | | | | | QTLRQLGLPVLLIAHRPETIASADR |
| | | | | | VLYLADGCFMELRHKRTIDDGLNKN |
| 6 | WP_017382355.1 | MULTISPECIES: | Enterobacter | 170 | MDTLKWTGRKQLPLTRQTESAECGL |
| | | peptidase | | | ACLVMMACWHGLQTDLPTLRERFSI |
| | | domain- | | | SSQGMTLQRLTECAAGIGLSSRAVR |
| | | containing | | | LELEDLRSLSLPCILHWNMNHFVVL |
| | | ABC | | | HKVRGSRLVIHDPEKGKVTLSLQDA |
| | | transporter | | | GKHFTGVALELMPASDFTARDERKK |
| | | | | | IHLRQLTGKTPGLLSAMSRIIVFAV |
| | | | | | ALEILTLGSPLLNQLVIDEVLVAAD |
| | | | | | QSLLTVIIIALLLMSLTQMLLSLAR |
| | | | | | QWASITLSVNFNMQWTARVFHHLVH |
| | | | | | LPLAWFDARSKGSINARFEAVDTIQ |
| | | | | | QALTTQVLEGILDVLLVVTALCMML |
| | | | | | LYSPEMTLIAVLAAIFYGVLRALWY |
| | | | | | PSLRQSAEDAWDAGAKESGHFLETL |
| | | | | | NGILSLRINGVTAHREAAWLNLNVA |
| | | | | | RRNTQLRQSRLLMCYDIAHTLTGSV |
| | | | | | VSAIILWKGAGEVLNGTFTVGMLVA |
| | | | | | YLSYQTRFSSSISSLTDKFFSWRML |
| | | | | | DVYNERLADIVLTPTEGLQQQPFRE |
| | | | | | HNNASHIPSVSRREMDEYAHAPLVL |
| | | | | | EHIIFSHKGGENPIIGGVSLTLNPG |
| | | | | | EVVAITGKSGCGKTTLVKLILGIHI |
| | | | | | PDEGRIRAFGILHTHPDYFQVRQRI |
| | | | | | GTVLQDDHLFKGSVADNIIFFSDDR |
| | | | | | NYERMIQCARLALIDSDVMAMPMGY |
| | | | | | QTLIGETGGGLSGGQKQRILLARAL |
| | | | | | YKKPGFLLLDEATSHLDVESEIRIS |
| | | | | | QTLRQLGLPVLLIAHRPETIASADR |
| | | | | | VLYLADGYLTELNLQHTVNNGESVC |
| | | | | | EYTNDVMAKN |
| 6 | WP_040107957.1 | peptidase | Klebsiella | 171 | MELMNWTGKKRLPLIRQTESAECGL |
| | | domain- | variicola | | ACLAMMACWHGLQTDLTTLRERFSI |
| | | containing | | | STQGMTLQRLIECAAGIQLSSRAVR |
| | | ABC | | | LEPEDLKSLSLPCILHWNMNHFVVL |
| | | transporter | | | HKVRRSRLVIHDPDKGKITLSLQDA |
| | | | | | GKHFTGVALELMPSSDFTVRDERKK |
| | | | | | IHLRQLTGKTPGLLAAMSRIITFAL |
| | | | | | SLEILTLGSPLLNQLVIDEVLVAAD |
| | | | | | RSLLTVVIVALLLLSLTQMLLSLAC |
| | | | | | QWASITLSVNFNMQWTARVFHHLVR |
| | | | | | LPLAWFDARSRGSVNARFDAIDTIQ |
| | | | | | QALTTQVLEGILDVLLVVTALCMML |
| | | | | | LYSPEMTLIAVLAAVIYGVMRTLWY |
| | | | | | PSLRQSAEDAWDAGARESGYFLETL |
| | | | | | SGILSLRINGVTTHREAAWLNLNVT |
| | | | | | RRNTQLRQSRLLMYYDIAHTLTGSV |
| | | | | | VSAIILWKGAGEVLNGTFTVGMLVA |
| | | | | | YLSYQMRFSSSISSLTDKFFAWRML |
| | | | | | DVYNERLADIVLTPTEGNQQQTAQE |
| | | | | | NNSTSTTPSIFQERVTEDTDLPLTL |
| | | | | | THIAFSHKGSNKPILRGVSLTLHTG |
| | | | | | EVVAITGKSGCGKSTLVKLILGIHL |
| | | | | | PNEGTIRTFGIPHTHPDYYQIRRRI |
| | | | | | GTVLQEDHLFRGSIADNIIFFSEDR |
| | | | | | NHERMIQCARLALIDSDIMTMPMGY |
| | | | | | QTLIGETGGGLSGGQKQRILLARAL |
| | | | | | YKKPGFLLLDEATSHLDVESEIQIS |
| | | | | | QTLRQLKVPVLLIAHRPETIASADR |
| | | | | | VLYLTDGYFTDLTHQYRNGSEYLVR |
| | | | | | MD |
| 6 | WP_065506285.1 | peptidase | Serratia | 172 | MDVTEMINWGWRKRLPLIRQTESAE |
| | | domain- | inhibens | | CGVACLAMIAGWHGHKVDMRTLRTE |
| | | containing | | | CHVSQLGMTFSQLMTCAEGLNLSGR |
| | | ABC | | | VIRLELDELHLLTVPCILYWDMNHF |
| | | transporter | | | TVLRRVRGQTLELHDPARGQLKMSL |
| | | | | | QEANRHFTGIAMELTPNHRFETQDR |
| | | | | | REKIRLTTLIGKTHGLKPALARIFC |
| | | | | | FAVALEVLALLGPLINQLVIDEVLV |
| | | | | | ALDASLLTLIVIAMLLMTGTQMLLE |
| | | | | | LARQWATLTMAVNFNMQWTANVFHH |
| | | | | | LLRLPIGWFEARNMGDISAKFDAVD |
| | | | | | TIQDTLTTTLLEAFLDVLLVVGTLI |
| | | | | | MMFFYSVKLTLVALAAAAVYALLRL |
| | | | | | CWFSTLRKAAQDSWNAGTEESSHFL |
| | | | | | ESLRGVLSLRVNGALMPRESAWRNL |
| | | | | | NVARRNAQLRESKLMMVYGIMQTVI |
| | | | | | GSVVAAAVLWLGAGSVLAGEFSVGM |
| | | | | | LVAYMSFQGRFSGSINGLIDKVVAY |
| | | | | | RMLDVYNERLADIVLTPREGAAASQ |
| | | | | | AGNQGPVCPVSAWPADLPVLSLEQV |
| | | | | | SFKYAGTEREILSQVSLDIMPGEVV |
| | | | | | ALVGASGSGKTTLAKLVLGLYPPSA |
| | | | | | GVIRTLGVEHRRADYQQTRQHIGAV |
| | | | | | LQEDQLFRGSIADNITFFSPKRDND |
| | | | | | KLGECVRLAQLDQDIEQLPMGYQTL |
| | | | | | ISEMGGTLSSGQKQRLLLARALYKE |
| | | | | | PKLLILDEATSHLDVTNEMLVSRTL |
| | | | | | RRLGLPILLIAHRPETIASADRVIE |
| | | | | | LSAGRIRTQSL |
| 6 | WP_048759058.1 | peptidase | Serratia | 173 | MDVTEGLNWGWRKRLPLIRQTESAE |
| | | domain- | liquefaciens | | CGVACLAMIAGWYGHRVDLPTLRAQ |
| | | containing | | | FKVSQLGMTFSQLIACAERLHLSGR |
| | | ABC | | | AVQLELEELHLLSLPCILYWDMNHF |
| | | transporter | | | TVLKRVRGQTLELHDPARGLVKMSL |
| | | | | | QEANRHFTGVAMELTPTHQFVVKDQ |
| | | | | | IKKIRLRELIGKTQGLKVALARIFC |
| | | | | | FALALEVFALIGPLINQLVIDEVLV |
| | | | | | ALDASLLTLIVIAMLLMAATQTLLE |
| | | | | | LARQWATLTMAVNFNMQWTANVFHH |
| | | | | | LLRLPINWFESRNMGDISAKFDAVD |
| | | | | | TIQDTLTTTLLEAFLDVLLVLGTLT |
| | | | | | MMFFYSVKLTLVALAAAMVYGLLRL |
| | | | | | FWFSTLRKAADDSWVAGTQESSHFL |
| | | | | | ESLRGVLSLRVNGALTPRETVWRNL |
| | | | | | NVARRNAELRESKLMMVYGIMQTVI |
| | | | | | GSLVGAAVLWLGAGAVLAGQFSVGM |
| | | | | | LVAYMSFQGRFSASISGLIDKAVAY |
| | | | | | KMLDVYNQRLADIVLTAREYAAAQA |
| | | | | | AGNSTLLLSASAWPQDRPALSLEQV |
| | | | | | SFHYAGTESEILSQVSLDIMPGEVV |
| | | | | | ALVGASGSGKTTLAKLVLGLYSPTA |
| | | | | | GVIRTLGIEHRQGDYSQVRQHIGVV |
| | | | | | LQEDQLFRGSIAENLSFFTPRVDQE |
| | | | | | KLVECARLAQLDQDIDNLPMGYQTL |
| | | | | | IGEMGGTLSAGQKQRLLLARALYKK |
| | | | | | PRLLILDEATSHLDVNNEMLISHTL |
| | | | | | RQLGLPILLIAHRPETIASADRIVE |
| | | | | | LAAGRLHRRKG |
| 6 | WP_149573319.1 | peptidase | Serratia | 174 | MDVTEGLNWGWRKRLPLIRQTESAE |
| | | domain- | liquefaciens | | CGVACLAMIAGWYGHRIDLPTLRAQ |
| | | containing | | | FKVSQLGMTFSQLIACAERLHLSGR |
| | | ABC | | | AVQLELEELHLLSLPCILYWDMNHF |
| | | transporter | | | TVLKRVRGQTLELHDPARGLVKMSL |
| | | | | | QEANRHFTGVAMELTPTHQFVVKDQ |
| | | | | | IKKIRLRELIGKTQGLKAALARIFC |
| | | | | | FALALEVFALIGPLINQLVIDEVLV |
| | | | | | ALDASLLTLIVIAMLLMAATQTLLE |
| | | | | | LARQWATLTMAVNFNMQWTANVFHH |
| | | | | | LLRLPINWFESRNMGDISAKFDAVD |
| | | | | | TIQDTLTTTLLEAFLDVLLVLGTLT |
| | | | | | MMFFYSVKLTLVALAAAMVYGLLRL |
| | | | | | FWFSTLRKAADDSWVAGTQESSHFL |
| | | | | | ESLRGVLSLRVNGALTPRETVWRNL |
| | | | | | NVARRNAELRESKLMMIYGIMQTVI |
| | | | | | GSLVGAAVLWLGAGAVLAGQFSVGM |
| | | | | | LVAYMSFQGRFSASISGLIDKAVAY |
| | | | | | KMLDVYNQRLADIVLTAREDAAAQA |
| | | | | | AGNATLLLSASAWPQDRPALSLEQV |
| | | | | | SFHYAGTESEILSQVSLDIMPGEVV |
| | | | | | ALVGASGSGKTTLAKLVLGLYSPTA |
| | | | | | GVIRTLGIEHRQGDYSQVRQHIGVV |
| | | | | | LQEDQLFRGSIAENLSFFTSRVDQE |
| | | | | | RLVECARLAQLDLDIDNLPMGYQTL |
| | | | | | IGEMGGTLSAGQKQRLLLARALYKK |
| | | | | | PRLLILDEATSHLDVNNEMLISHTL |
| | | | | | RQLGLPILLIAHRPETIASADRVVE |
| | | | | | LAAGRLHRRKG |
| 6 | WP_004944116.1 | peptidase | Serratia | 175 | MDVTEMINWGWRKRLPLIRQTESAE |
| | | domain- | plymuthica | | CGVACLAMIAGWHGHRVDMRTLRAE |
| | | containing | | | CHVSQLGMTFSQLMACAEGLNLSGR |
| | | ABC | | | VIRLELDELHLLAVPCILYWDMNHF |
| | | transporter | | | TVLRRVRGKTLELHDPARGLLKMTL |
| | | | | | QEANRHFTGIAMELTPTHRFETRDR |
| | | | | | REKIRLTTLIGKTHGLKPALARIFC |
| | | | | | FAVALEVLALLGPLINQLVIDEVLV |
| | | | | | ALDASLLTLIVIAMLLMTGIQMLLE |
| | | | | | LARQWATLTMAVNFNMQWTANVFHH |
| | | | | | LLRLPIGWFEARNMGDISAKFDAVD |
| | | | | | TIQDTLTTTLLEAFLDVLLVVGTLS |
| | | | | | MMFFYSAKLTLVALAAAAVYGLLRL |
| | | | | | CWFSTLRKAAQDSWNAGTEESSHFL |
| | | | | | ESLRGVLSLRVNGALMPRESAWRNL |
| | | | | | NVARRNAQLRESKLMMVYGIMQTVI |
| | | | | | GSLVAAAVLWLGAGSVLAGEFSVGM |
| | | | | | LVAYMSFQGRFSASINGLIDKAVAY |
| | | | | | RMLDVYNERLADIVLTPREGATALQ |
| | | | | | AGNKGPAYPAGDWPPDLPVLSLEQV |
| | | | | | SFKYAGTEREILSQVSLDIMPGEVV |
| | | | | | ALVGASGSGKTTLAKLVLGLYPLSG |
| | | | | | GVIRTLGLEHRRTDYQQIRQHIGAV |
| | | | | | LQEDQLFRGSIADNITFFSPKMDSE |
| | | | | | KLGECARLAQLDRDIEQLPMGYQTL |
| | | | | | ISEMGGTLSSGQKQRLLLARALYKE |
| | | | | | PKLLILDEATSHLDVANEMLVSRTL |
| | | | | | RQLGLPILLIAHRPETIASADRVIE |
| | | | | | LTAGRLRTLGL |
| 6 | WP_086046966.1 | peptidase | Vibrio | 176 | MRVVKDVYRKWEFGKKLSLVRQTEK |
| | | domain- | alginolyticus | | AECGVACLAMIADWYGYKIDLRDLR |
| | | containing | | | AKFGITHHGMSFKRLIECGESLNLH |
| | | ABC | | | AQAKGGVELHHLKELATPCILHWDS |
| | | transporter | | | NHFVVLKAVKKDCVIVHDPARGECV |
| | | | | | FSFTELNKHFTGVVLELTPTDDFKQ |
| | | | | | RDESKHIKLSRLIGKTHGLKRALGK |
| | | | | | ILVLAIILEGLALLLPIMNQVVIDE |
| | | | | | VLVGYDEDLLVLVILGILLITAAQL |
| | | | | | VVSIVKEWATALMSVDFNMQWLANV |
| | | | | | FHHLFRLPIDWFEKREIGDIAAKFD |
| | | | | | AVGTIQHTLTTSVIQAFLDLILVLG |
| | | | | | TLSVMMFYSPLLSCIAMFAAILYII |
| | | | | | LRMVWYGAFKRAEENTWETSTKENS |
| | | | | | YFLETINGLLSLRVNGALNWRENIW |
| | | | | | RNLNIDRRNAQLHEMKLGMVYSVVS |
| | | | | | TTINSLVSAAVLWFGANLVISGDFS |
| | | | | | IGMLVAYLSYQGRFSGSVSSLIDKF |
| | | | | | FEYKMLSVYNERLADIVLTEREFDS |
| | | | | | ISVDKVECFTQVVGQDDDILQFQNV |
| | | | | | SYSYAPDMPLILNSASFDIKGNEVV |
| | | | | | ALVGESGCGKSTVSKLILKLYQPTT |
| | | | | | GNIIWFGNKGINPIEIRSRVGVVLQ |
| | | | | | DDSLFGGSIIDNITLSAEAIDEEWM |
| | | | | | YECAQRARVHEDISRLNMGYHTLLG |
| | | | | | EQGGGLSGGQKQRILIARALYKKPN |
| | | | | | LLILDEATSHLDIDTERFVCSELRS |
| | | | | | LNIPTLMIAHRPDTIAAADRVLLLQ |
| | | | | | DGLITELVRNQ |
| 6 | WP_086957993.1 | peptidase | Vibrio casei | 177 | MHAEGLIDWGWGKKLPLIRQTEKAE |
| | | domain- | | | CGIACLAMVADWHGYKTDLRSLRVK |
| | | containing | | | CGITQHGMSFARLIECGALLKLSGR |
| | | ABC | | | AVRLDLHELQQLATPCILHWNLNHF |
| | | transporter | | | VVLKKVTGKKIEIHDPANGALALSH |
| | | | | | DEVNKHFTGIALELTPTHDFEEKVE |
| | | | | | SKAIRLSTLIGKTVGLKGALGRIFV |
| | | | | | FALVLEVLALTLPIFNQIVIDEVLV |
| | | | | | GYDKNLLVLVIGAILMVTATQTLIG |
| | | | | | LAQQWATIKLSVNFNMQWAANVFHH |
| | | | | | LFRLPIDWFEKRDIGSISAKFLAVD |
| | | | | | VIQQTLTTSVIQALLDLVLVVGTLM |
| | | | | | VMLVYSPQLSLIAIAAALGYIALRL |
| | | | | | AWFGAFKRAEENTWEASTQEESYFI |
| | | | | | ETVRGVLSLRVNGTLPWRESSWRNL |
| | | | | | NINRRNAQLHEQKLGMIYSTMNVAI |
| | | | | | VSLVSASVLWFGANLVLDGLFTIGM |
| | | | | | LMAFLSYQGRFSASISSLTDKYFEF |
| | | | | | KMLSVYNERLADIVLTEKETDSDSN |
| | | | | | VIGQTNVTNNPNDNVIEFVDVYFSY |
| | | | | | GKDQSPILNGASFSVKSNEIVALVG |
| | | | | | PSGCGKSTISKLLLGIYSATSGSIY |
| | | | | | YFGDKSMASKPLRTQVGAVLQEDQL |
| | | | | | FSGSIIENITFFAGDLDEEWLVECA |
| | | | | | RRAGVHDDIERLNMGYHTLIGEMGS |
| | | | | | SLSGGQKQRVLIARALYKKPKFLVL |
| | | | | | DEATSSLDIYTESFVCQMFKEIKIP |
| | | | | | ILMIAHRPETIAAADRVLLMDGGLV |
| | | | | | QEIPNDFRREVQDVSL |
| 7 | V_PCAT_cvaB | peptidase | Escherichia | 178 | MTNRNFRQIINLLDLRWQRRVPVIH |
| | | domain- | coli | | QTETAECGLACLAMICGHFGKNIDL |
| | | containing | | | IYLRRKFNLSARGATLAGINGIAEQ |
| | | ABC | | | LGMATRALSLELDELRVLKTPCILH |
| | | transporter | | | WDFSHFVVLVSVKRNRYVLHDPARG |
| | | | | | IRYISREEMSRYFTGVALEVWPGSE |
| | | | | | FQSETLQTRISLRSLINSIYGIKRT |
| | | | | | LAKIFCLSVVIEAINLLMPVGTQLV |
| | | | | | MDHAIPAGDRGLLTLISAALMFFIL |
| | | | | | LKAATSTLRAWSSLVMSTLINVQWQ |
| | | | | | SGLFDHLLRLPLAFFERRKLGDIQS |
| | | | | | RFDSLDTLRATFTTSVIGFIMDSIM |
| | | | | | VVGVCVMMLLYGGYLTWIVLCFTTI |
| | | | | | YIFIRLVTYGNYRQISEECLVREAR |
| | | | | | AASYFMETLYGIATVKIQGMVGIRG |
| | | | | | AHWLNMKIDAINSGIKLTRMDLLFG |
| | | | | | GINTFVTACDQIVILWLGAGLVIDN |
| | | | | | QMTIGMFVAFSSFRGQFSERVASLT |
| | | | | | SFLLQLRIMSLHNERIADIALHEKE |
| | | | | | EKKPEIEIVADMGPISLETNGLSYR |
| | | | | | YDSQSAPIFSALSLSVAPGESVAIT |
| | | | | | GASGAGKTTLMKVLCGLFEPDSGRV |
| | | | | | LINGIDIRQIGINNYHRMIACVMQD |
| | | | | | DRLFSGSIRENICGFAEEMDEEWMV |
| | | | | | ECARASHIHDVIMNMPMGYETLIGE |
| | | | | | LGEGLSGGQKQRIFIARALYRKPGI |
| | | | | | LFMDEATSALDSESEHFVNVAIKNM |
| | | | | | NITRVIIAHRETTLRTVDRVISI |
| 7 | WP_001183604.1 | MULTISPECIES: | Enterobacteriaceae | 179 | MQQDRSMTNRNFRQIINLLDLRWQR |
| | | peptidase | | | RVPVIHQTETAECGLACLAMICGHF |
| | | domain- | | | GKNIDLIYLRRKFNLSARGATLAGI |
| | | containing | | | NGIAEQLGMATRALSLELDELRVLK |
| | | ABC | | | TPCILHWDFSHFVVLVSVKRNRYVL |
| | | transporter | | | HDPARGIRYISREEMSRYFTGVALE |
| | | | | | VWPGSEFQSETLQTRISLRSLINSI |
| | | | | | YGIKRTLAKIFCLSVVIEAINLLMP |
| | | | | | VGTQLVMDHAIPAGDRGLLTLISAA |
| | | | | | LMFFILLKAATSTLRAWSSLVMSTL |
| | | | | | INVQWQSGLFDHLLRLPLAFFERRK |
| | | | | | LGDIQSRFDSLDTLRATFTTSVIGF |
| | | | | | IMDSIMVVGVCVMMLLYGGYLTWIV |
| | | | | | LCFTTIYIFIRLVTYGNYRQISEEC |
| | | | | | LVREARAASYFMETLYGIATVKIQG |
| | | | | | MVGIRGAHWLNMKIDAINSGIKLTR |
| | | | | | MDLLFGGINTFVTACDQIVILWLGA |
| | | | | | GLVIDNQMTIGMFVAFSSFRGQFSE |
| | | | | | RVASLTSFLLQLRIMSLHNERIADI |
| | | | | | ALHEKEEKKPEIEIVADMGPISLET |
| | | | | | NGLSYRYDSQSAPIFSALSLSVAPG |
| | | | | | ESVAITGASGAGKTTLMKVLCGLFE |
| | | | | | PDSGRVLINGIDIRQIGINNYHRMI |
| | | | | | ACVMQDDRLFSGSIRENICGFAEEM |
| | | | | | DEEWMVECARASHIHDVIMNMPMGY |
| | | | | | ETLIGELGEGLSGGQKQRIFIARAL |
| | | | | | YRKPGILFMDEATSALDSESEHFVN |
| | | | | | VAIKNMNITRVIIAHRETTLRTVDR |
| | | | | | VISI |
| 8 | VC1_PCAT_cvaB | peptidase | Vibrio cholerae | 180 | MNSSEFAINVSKLLEFGRIKRVPTI |
| | | domain- | | | LQSEVDECGISCLAMVSSYYGNKIN |
| | | containing | | | LPPLRRDSNFSNEGMKLIEVMNVAN |
| | | ABC | | | SLNLTSRALQCSLDDTGKLKLPCIL |
| | | transporter | | | HWELNHYVVLTDIIKGNYYINDPAI |
| | | | | | GRRILTTKQFSDSFTGIALELTPNS |
| | | | | | EFKTSDSRVVMGIGQLWNKLLGIKR |
| | | | | | SLFSLFILTIILQFFALLSPYYMQW |
| | | | | | VIDHVLLSHDMPLLAILALGFSLLK |
| | | | | | VIQIIVSSFRTWLIIRISSSLNIQM |
| | | | | | GANLFNHLLRLPISFFEKRHVGDII |
| | | | | | SRFGSLNIIKQMISTGFVEGLIDGI |
| | | | | | MAIIVLIMMYIYSPILANLVVVIIF |
| | | | | | IYFLIQCFFYNPTRTIEEELINSEA |
| | | | | | KEDSIFLESVRGIQSIKLFTLEATR |
| | | | | | LNTWLNKYADVININIRKSKIGICQ |
| | | | | | NILTSLLFGLESILIVYIGAVTVMD |
| | | | | | GKLTVGMLLAFIAYKSHFISSISSF |
| | | | | | IGKCIAFKMLSLHLERLSDIALETE |
| | | | | | ETKISSINSDFFGKSNGFLRVENLG |
| | | | | | FRYSEKSDWVFNGVSFEISPGECLA |
| | | | | | ITGVSGCGKTTLMKVILGLLKPTEG |
| | | | | | KIYLDGVESSQWPLDLYRKKFSAVM |
| | | | | | QKDTLFSGTLIENITMFEQDYDENR |
| | | | | | LVDCCRMACILDDIRALPMGFHSLV |
| | | | | | GDMGSSFSGGQLQRVYLARALYRNP |
| | | | | | LILCLDESTSHLDMSNESLINENLK |
| | | | | | DTFFTKIIIAHREETISTADKIYRL |
| | | | | | DKI |
| 8 | WP_086933435.1 | peptidase | Agarilytica | 181 | MYFFGRLENSIKKKLPLIMQNEASE |
| | | domain- | rhodophyticola | | CLLACIAMILNYHGRKVDLIELRDS |
| | | containing | | | INISSRGSGFSHLLNISNKLNIKTR |
| | | ABC | | | PVKVDIKSINKLSLPCILHWKKNHF |
| | | transporter | | | VVLKSINKSGIYIHDPSIGKRFVIY |
| | | | | | KNLSEFFFGIAAEVTPTENFKKKDK |
| | | | | | KENKDIKKFFKDFSGIKRNILYIIL |
| | | | | | LSFFLQLISLSTPFYIQTIIDKVIQ |
| | | | | | SHNIKLLEILFLCFIFLIIIQSIIS |
| | | | | | YSRSYITNLFSFKYGFHSSSRLFYH |
| | | | | | LIRLPTLYFSRRGLGDVITKFEAQG |
| | | | | | YLKNYLSNEVVSSFIDAIMLIIAII |
| | | | | | AMLFYSVKLSIIVFAVSVFYVVLDF |
| | | | | | NNFKIQKVLLEDAVSFGAKGHTAFI |
| | | | | | ESVRLIESVKILENEKNRHYKWLTY |
| | | | | | FSQETDKRYESAHLENKIEFLRQII |
| | | | | | FGLENITIIYLGSLMVIKNSISLGM |
| | | | | | LIAFISFKVKYVDTVKSLMRQIMSL |
| | | | | | NTLNVYMNRLSDIVRAKTDINTSGS |
| | | | | | KSYFGDDNDDKTSCISKFQRRNEYI |
| | | | | | ISGKIEVINLAFRYGDSENFVFRNI |
| | | | | | NFTIKQGEKVVIVGGSGCGKTTLLK |
| | | | | | CLMGLYLPTEGTILIDGKPLAEVGN |
| | | | | | YRSQISGVLQNDSLFSGTIAENISG |
| | | | | | FDPLPDLVKIRNCAKLSSINIDIDS |
| | | | | | MPAGYNTYLGDMGGGLSGGQMQRII |
| | | | | | IARALYSNPSILFMDEATSHLDVGN |
| | | | | | ENIVNTNLNNLNITQVLVAHRMETV |
| | | | | | MSAERQINISNFN |
| 8 | WP_014950962.1 | peptidase | Alteromonas | 182 | MMNITHPLSTQILYTTKRVPVVLQS |
| | | domain- | macleodii | | EAAECGLACMTMIAQYYSDKRDLNA |
| | | containing | | | LRQTVSVSLRGTTLKDVMRIASDLG |
| | | ABC | | | FQTRAIKIEMEHLAQLSCPAILHWN |
| | | transporter | | | MNHFVVLTKVKGKTLTIHDPALGER |
| | | | | | KLTYSEASKYITGIALEVTPSDTFS |
| | | | | | PKKSAPRLGLSQFFTRTTGFKRNLL |
| | | | | | TLFALSIVLQLFALASPYYMQTVVD |
| | | | | | DVLIYNNDALLKALAIGFALLLIIE |
| | | | | | TFTSGIRKFVILSVSSRLQLQMSAS |
| | | | | | VFKHLLSLPLDYFDKRHIGDVVSRF |
| | | | | | GSLASIREFLTTGLVTALLDGLMAV |
| | | | | | ITLAVMILYSPKLTLVVVAIMLAYL |
| | | | | | AVRLGLLPFIKRLTTERIALAALEQ |
| | | | | | SHFMESVRAILPIRVYSQEVQRHGQ |
| | | | | | WQNKLVATLNKDITLGKINIGSALT |
| | | | | | NQLLFGAENLVVVYIGASLVMEGTL |
| | | | | | SIGMLLAFMAYKARFVSALDGVVNK |
| | | | | | LIELSMLGVHFNRLSDILLTPSQHK |
| | | | | | PLSLKAK |
| 8 | WP_075607215.1 | peptidase | Alteromonas | 183 | DDTFEKQNVHFSNADVPNSSTSNSL |
| | | domain- | pelagimontana | | APNVLTPDGASTPSLLDAKTRPVAL |
| | | containing | | | KATALSYRYSETNAWVFKNLMFSVH |
| | | ABC | | | SGEIVAITGDSGCGKSTMLKCLMGL |
| | | transporter | | | YPVSEGNIEHPSSTNPVIASVLQED |
| | | | | | TCLSGTIAQNICCFEEAPDLKKMVY |
| | | | | | VAQLACIHEDIMQMPMQYHSLVGDM |
| | | | | | GSSLSGGQKQRLLLARALYQEPDIL |
| | | | | | FLDEASSHLDMENEAQINHHLKSLN |
| | | | | | MTRIIVAHRPQSIAMADKVYRLENG |
| | | | | | NLHLCPPTEVTGSATSGKQGETLMT |
| | | | | | AIPLNLPPLSLSIIRRRAVPMVFQS |
| | | | | | EAAECGLACLCMIARYFKDGSDMTE |
| | | | | | LRQKYGVSLRGATLKSIMDMAADMG |
| | | | | | LSARALKLETEHVKQLTLPCILHWD |
| | | | | | LQHFVVLTKVSQRGVVIHDPALGER |
| | | | | | ALNWDDVGNHFTGIALEITPTSEFK |
| | | | | | PARQAPTLTLRHFWQRIVGLKRHLL |
| | | | | | ILFALSALIQLFAIASPYYMQTVID |
| | | | | | DVLLRQQRDLLLVLALGFGMLLLIE |
| | | | | | SATSVLRRMAILALSSRLHQQMSAN |
| | | | | | VFHHLIRLPVSYFSKRHMGDVVSRF |
| | | | | | GSLSQVRELLTVGVVTAVLDGLLAL |
| | | | | | VTLAVMMVYSVTLSGIVAVVVLIYL |
| | | | | | AARIALISTMKRLNAERISIAAKEQ |
| | | | | | SHFMESIRAIQTVKQFGQEMNRQSQ |
| | | | | | WQNKLADTINTDIRLGKWDISVSCL |
| | | | | | NQLLFGFENILIIYFAATEVMDNAM |
| | | | | | TVGMLYAFIAYKTRFITAIDGLINK |
| | | | | | AIEFKMVAVHFERLADIVFTKQESF |
| | | | | | LQTAPSLPQLSDNIIDAPTRPLVIK |
| | | | | | DLCYRYSPADAPVFENINLTIEAGS |
| | | | | | TVAIVGPSGAGKTTLLKCLMGLLPP |
| | | | | | SQGDVLYGGDSVFSAGVFRQLTSSV |
| | | | | | LQDDNCLSGSIMDNITCFTDSPDFE |
| | | | | | RMVQAALKACIHEEIMAMPMQYQTL |
| | | | | | VGDMGSTLSGGQLQRLLLARALYRQ |
| | | | | | PKILFLDEASSHLDLNNEAAINQHL |
| | | | | | KALAITRVVVAHRSETIAMADRIYL |
| | | | | | LRNGTLCEVPHSAVLSSPSPTSKNP |
| | | | | | PGENYD |
| 8 | WP_011145222.1 | MULTISPECIES: | Photorhabdus | 184 | MMKLEKIIFWHSHNLPSILQTEIAE |
| | | peptidase | | | CGLACLASISSYHGYQVDLSSLRKK |
| | | domain- | | | FRIPLTGTNLNDIAYYAKELKLSYR |
| | | containing | | | AVKLDINEINQLKLPCILHWELNHF |
| | | ABC | | | VVLKKISKNTITIMDPSIGFRRLSY |
| | | transporter | | | NEFSNCFTGIAVEFWPNNDFKKKID |
| | | | | | KEKVDILSFFKNISGIKRSLVKILL |
| | | | | | LAFVLEFFALIGPFYMQLIIDHGIV |
| | | | | | SEDKNLILLLSIGFGVLLVFEQVIN |
| | | | | | IIQSLLTTYISTNLNIQWKANVFTH |
| | | | | | LMNLPVSFFEKRHLGDIISKFSSID |
| | | | | | SIQNTLTSSFFISICNSLVSATTII |
| | | | | | IMTMYNFQLTLISIVTIFIYIIIRI |
| | | | | | IWYYPLKEATKENIVQSAKLSSNFM |
| | | | | | ETMRGIRAIKIFCKENHRYNSWLTL |
| | | | | | SINTVNSALKSQRLSLGFSFSNRII |
| | | | | | FGIQNILIVYIGTSFIFDNTFTVGI |
| | | | | | LISFLAYKTQFESRTISLIDLFISF |
| | | | | | KMLGIHIERLSDIVLSETELKSNIL |
| | | | | | LKTETIKSNKIEIKNLSFKYEGNNN |
| | | | | | YLIENLYFDIQPGDSVAIIGPSGCG |
| | | | | | KSTLLNLMIGNILPTGGEVRIGGVI |
| | | | | | VNKTHPKIIRDNIGYVAQDDSLFSG |
| | | | | | SIMENITFFDEHPDTDKAINCAKIA |
| | | | | | AIHHDIKKMPMEYETLIGDMGTILS |
| | | | | | GGQKQRLCLARALYKDPKILFLDEA |
| | | | | | TSHLDTENEKIISSNLKMLSITKVM |
| | | | | | VAHRKETIESANKLIDLTKKCA |
| 8 | WP_011074382.1 | peptidase | Shewanella | 185 | MTIATDKHAALMASESNSPVDLLEF |
| | | domain- | oneidensis | | SGNKRVPLILQAEMAECGLACMAMI |
| | | containing | | | ASFNGHKLDMAALRKRFTANLKGMN |
| | | ABC | | | LQQLISLGDSIGLSSRALKCPLEEV |
| | | transporter | | | GKLALPCILHWDMNHFVVLTGVTKK |
| | | | | | SISINDPAAGKRTLSLQEFAKHFTG |
| | | | | | IALELTPTKAFVKQDERQQMRLSQL |
| | | | | | WTKISGVNAALITLLLLSVLLQVFA |
| | | | | | LVTPYYMQWVVDEVLVSQDQPLLIV |
| | | | | | LAIGFGLLVVINVFTTGVRSWLVLR |
| | | | | | VSSLLNMQMGVNLLRHLLRLPMNYF |
| | | | | | EKRHIGDLVSRFGSLAQVRERLTTG |
| | | | | | LVETVVDGVMSIAVLVMMLIYSVKL |
| | | | | | TLVVMAAVALYTLMRFALYRPLHRA |
| | | | | | TEESIQAKAKEQSNFLENIRGIQTI |
| | | | | | KLFTCESARQGIWQNRYSEVINADI |
| | | | | | RLGRLKISFDAMNKLLFGVENIIVI |
| | | | | | YMAAMIVMSGGLTIGMVLAFIAYKN |
| | | | | | QMTERVASLIEQLIMFRMLRLHLDR |
| | | | | | ISDIALHEQEAHQEGFTPLNVVKGR |
| | | | | | LSLENVSFRYGENEPEVVSNLSLDI |
| | | | | | QAGESVAIVGASGCGKTTLVKLMLG |
| | | | | | LLVPSSGRILLDGQAIQQIGLTQYR |
| | | | | | QQIAAVMQDDTLLSGSIADNITFFD |
| | | | | | PEPNYVKMQQCAQLAVIDMDIAHMP |
| | | | | | MGYNSLVGDMGNQFSGGQVQRLLLA |
| | | | | | RALYQSPSILFMDEATSHLDIMNEA |
| | | | | | KISEQIKNLNMTRIIIAHRPETIKQ |
| | | | | | ADRVVVMHQGKIMTAEELQQAQSAS |
| 8 | WP_014611435.1 | peptidase | Shewanella | 186 | MTIAAEKQAALAINEHNSPVDLLEF |
| | | domain- | putrefaciens | | SGKKRVPLILQAEMAECGLACMAMI |
| | | containing | | | ATFNGHKLDMAALRKRFTANLKGMN |
| | | ABC | | | LQQLISLGDSIGLSSRALKCPLQDV |
| | | transporter | | | GKLALPCVLHWDMNHFVVLTGVTKK |
| | | | | | SISVNDPAVGKRTLSLQEFAKHFTG |
| | | | | | IALELTPTKAFVKQDERQQMHLSQL |
| | | | | | WTKISGVNAALITLLLLSVLLQVFA |
| | | | | | LVTPYYMQWVVDEVLVSQDQPLLIV |
| | | | | | LAIGFGLLVLINVFTTSVRSWLVLR |
| | | | | | VSSLLNMQMGVNLLRHLLRLPMSYF |
| | | | | | EKRHIGDLVSRFGSLAQVRERLTTG |
| | | | | | LVETVVDGVMSIAVLVMMLIYSIKL |
| | | | | | TLVVMAAVALYTLMRFALYRPLHRA |
| | | | | | TEESIQAQAKEQSNFLENIRGIQTI |
| | | | | | KLFTCESARQGIWQNRYSEVINADI |
| | | | | | RLGRLKISFDAMNKLLFGVENIVVI |
| | | | | | YLAATIVMSGGLTIGMVLAFIAYKN |
| | | | | | QMTERVASLIEQLIMFRMLRLHLDR |
| | | | | | ISDIALHEQEAHQESFTQLNIVKGR |
| | | | | | LSLENVSFSYGMNEPEVISNLSLDI |
| | | | | | QAGESVAIVGASGCGKTTLVKLMLG |
| | | | | | LLVPTDGRILLDGQAIQQIGLTQYR |
| | | | | | QQIAAVMQDDTLLSGSVADNITFFD |
| | | | | | PEPNYVKMQQCAQLAAIDMDIAHMP |
| | | | | | MGYNSLVGDMGNQFSGGQVQRLLLA |
| | | | | | RALYQSPSILFMDEATSHLDIMNEA |
| | | | | | KISEQIKNLDMTRIIIAHRPETIKQ |
| | | | | | ADRVVVMHQGKIMTAEELQQAQSAS |
| 8 | WP_074052065.1 | peptidase | Vibrio harveyi | 187 | MANSNVGHSSSSNFNAKTLLNFNGV |
| | | domain- | | | KKVPLVLQSENTECGLACIAMISSF |
| | | containing | | | YGHKINLLPLRHHTNLGDQGVELLR |
| | | ABC | | | LMDIANDLSLVPRALQCSLEEVTHL |
| | | transporter | | | ALPCILHWNLDHYVVLTKIKKGRYY |
| | | | | | INDPAGGEVIVNKKQFSDAFTGIAL |
| | | | | | ELTPNRNFKKEDSRVLMKIEQLWGR |
| | | | | | IIGIKRSLIALFSLSILIQILALLS |
| | | | | | PYYMQWIIDNVLLSHDKPLLLVLAI |
| | | | | | GFSFLKIIQVFISSFRSWFMIRLNC |
| | | | | | EISIQLASNIFDHLIRLPISFFEKR |
| | | | | | HVGDIVSRFGSFNAIKEMITTGLVE |
| | | | | | AIIDGLMAVIILFMMYIYSPILSSF |
| | | | | | VLVFISISLIVQYAFFYPSKRLEEE |
| | | | | | SIVFEAKEDTMFLESVRSIQTLKLF |
| | | | | | GQETNRLNGWLNKYADVTNIGIRKS |
| | | | | | RLNLTENIITTLLNGFESILILYLG |
| | | | | | ALIVIDGELTIGMLLAFIAYKSQFT |
| | | | | | SNISALVGKWLSFKMLSLHLERLSD |
| | | | | | ITLEDKEKLNFSSEQIKTSGSTIRV |
| | | | | | EGLGFRYSESSEWIFRDVSFEVESG |
| | | | | | SSIAIVGPSGCGKSTLMKVILGLLE |
| | | | | | PTEGKIFFNDIDISQLSLSNYRQQF |
| | | | | | SAVMQNDSLLSGTIAENITMFDLKF |
| | | | | | EQSKLEECCKKACIFDEIINLPMRF |
| | | | | | NSLVGDMGSVFSGGQLQRIYLARAL |
| | | | | | YRDPKVLCLDESSSHLDKANEKKVN |
| | | | | | QSLRELDITKIIIAHSAETINMADR |
| | | | | | VINLDGYSH |
| 9 | ER14_PCAT_cvaB | peptidase | Pantoea | 188 | MPFFHQAESSECGLACLAMISSYYG |
| | | domain- | agglomerans | | LHKDLFSLRSRYMTPASGARLADVI |
| | | containing | | | QIAEDSGMLTRALSLEVSELKLLKL |
| | | ABC | | | PCILHWDFNHFIVLVKTTKKDFIVH |
| | | transporter | | | DPASGRLKLTRKEIEENFTGVALEI |
| | | | | | FPCSDYQERADDKAERIEITKLTKN |
| | | | | | IKHLTSNLLKIFFLSVVIEAIILLI |
| | | | | | PIGTQLTMDYVLPSADQSLLTLICT |
| | | | | | GLFIFILLRSLLSSVRAWLTLKIGS |
| | | | | | TIDVQWQMNLYKHLINIPLSYFERR |
| | | | | | KTGDVQSRFSSIETIRSTIVSGITG |
| | | | | | AIIDTIMCMILLLLMFLYSGLLSVI |
| | | | | | ATGITAVYVIIRIVSYRFYKRLQEE |
| | | | | | SLVKSARASSHFMETLYGISAVKAH |
| | | | | | GLKNGRVSQWINLKIDSINANLQIS |
| | | | | | KVDMVYSGVNTFVSAVDQVTLLWVG |
| | | | | | ANLVIHNEISLGMFIAFGTFRSQFS |
| | | | | | DRIASLTGYAFNYKLLDLHSERLSD |
| | | | | | IAMQKQEGETAHDAIIRHTEPSDPA |
| | | | | | ELVVECISYSYNQFSRPVLKDISFT |
| | | | | | VEPGESVAIVGSSGSGKTTLMKLIS |
| | | | | | GLIRPEKGRILIDRTDIHKSSSGKV |
| | | | | | NARLGCILQDDKLFSGSIRENITCF |
| | | | | | SEVPDEEWMIDCARKALIHDVIMTF |
| | | | | | PMGYDTLLGEVGEGLSGGQKQRVYI |
| | | | | | ARALYKRPNILVMDEATSALDNVSE |
| | | | | | KLVSNSINSLRITKVIIAHRESTIA |
| | | | | | GADRIIRLDELNT |
| 10 | ER31_PCAT_cvaB | peptidase | Pantoea allii | 189 | MNEELHTILRGLLQFSGRGRVPLHL |
| | | domain- | | | QTETSECGLACLAMVAGFHGSHTDL |
| | | containing | | | LTLRERSGLSSRGSTLTSLISVAGE |
| | | ABC | | | MNLAARALLLDLDSLSELRKPCILH |
| | | transporter | | | WDLNHFVVLVSVKGKRCIVHDPAAG |
| | | | | | RQSMSLEDLSSHFTGVALELWPDSD |
| | | | | | FKPDIRQKKLKVSTLLNSITGFQQS |
| | | | | | LVKIFCLSLMIEFITLLMPVATQMV |
| | | | | | MDNAVPAADTGLLTLICLSLLLLTL |
| | | | | | LQACLGLFRGWTMMVMGIYTDLQWK |
| | | | | | DGLYRHLLRLPLSWFEKRRIGDIQS |
| | | | | | RFASLDTLRNTFTQSINGAVIDGVM |
| | | | | | ATGALILLILYGGWLAWVVTGFTLI |
| | | | | | FVLLRVFTWPRYRQAQEELLIKNAR |
| | | | | | AASSFTETLYAAATVRAQGLSHRRR |
| | | | | | ETWLNMMADATGSGVSLLRFDMLAG |
| | | | | | IAGTFIAASDSVIILWLGISAVINH |
| | | | | | TMTLGAFVAFSAFRGMFSERILSLT |
| | | | | | GVALQLRMLSLHNERIADIALSEPE |
| | | | | | GDKEDEKRLFPEGEALALKCEALMF |
| | | | | | RYDRFSPPVLQNLNISIGAGESVAI |
| | | | | | TGASGRGKTTLMKILCGLTTPVKGR |
| | | | | | VLVNNVDIKAAGLGNYRRSVACILQ |
| | | | | | DDRLLAGTLRDNITGFSHDVDEDWM |
| | | | | | VDCARLSHIHDDIMALPMGYETLTG |
| | | | | | ELGEGLSGGQRQRLFIARALYRRPG |
| | | | | | IIFMDEATSHLDEKNEALINAAMQT |
| | | | | | LKMTRVLIAHRPSTISSADRIIEL |
| 11 | L_PCAT_mclB | peptidase | Escherichia coli | 190 | MTNGNFRQIINQLDMRWRRRVPVIH |
| | | domain- | | | QTETAECGLACLAMICGHFGKNIDL |
| | | containing | | | ISLRRKFNLSARGANLAGINGIAEQ |
| | | ABC | | | LGMVTRALSLELDELGALKMPCILH |
| | | transporter | | | WDFSHFVVLVSVKRNRYVLHDPARG |
| | | | | | RRYLGREEMSRYFTGIALEVWPGSE |
| | | | | | FQAETQQTRISLRSLINSIYGIKRT |
| | | | | | LAKIFCLSVVIEAINLVMPVGTQLV |
| | | | | | MDHAIPAGDRGLLTLISAGLMFFIL |
| | | | | | LRAAVSMLRAWSSLVMSTLINVQWQ |
| | | | | | SGLFDHLLRLPLAFFERRKLGDIQS |
| | | | | | RFDSLDTLRATFTTSVIGLIMDSIM |
| | | | | | VVGVFVMMLLYGGYLTWIVLCFTTI |
| | | | | | YIFIRLVTYGNYRQISEECLVREAR |
| | | | | | AASYFMETLYGIATVKIQGMVGIRG |
| | | | | | AYWLNMKIDAINSGIKLTRMDLLFG |
| | | | | | GINTFVTACDQVVILWLGAGLVIDN |
| | | | | | QMTIGMFVAFSSFRGQFSERVTSLT |
| | | | | | SFLLQLKIMSLHNERIADIALHEKE |
| | | | | | EKKHEIEIVAHMGPISLETNDLSYR |
| | | | | | YDSQSAPIFSALSLSVAPGESVAIT |
| | | | | | GASGAGKTTLMKVLCGLFEPDSGRV |
| | | | | | LINGIDIRQMGINNYHRMIACVMQD |
| | | | | | DRLFSGSIRENICGFAEEMDEEWMV |
| | | | | | ECARASHIHDVIMNMPMGYETLIGE |
| | | | | | LGEGLSGGQKQRIFIARALYRKPGI |
| | | | | | LFMDEATSALDSESEHFVNVAIKNM |
| | | | | | NITRVIIAHRETTLKTVDRVISI |

In some embodiments, the PCAT comprises an amino acid sequence that has about 70% or greater (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity to any of the amino acid sequences described herein (e.g., the amino acid sequence of E coli CvaB). For example, the PCAT may contain an amino acid sequence that has about 70% or greater (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity to SEQ ID NOS: 44, 62, 64, 68, 124, 143, 144, 165, 169, 176, 178, 180, 181, 188, 189, or 190. Additional amino acid residues may be present at the N-terminus or C-terminus of any of these sequences (e.g., a starting methionine (“M”) residue at the N-terminus, or a sequence containing a purification tag at the C-terminus).

### C. Membrane Fusion Proteins

Gram-negative bacterial cells according to the present disclosure also contain nucleic acids encoding membrane fusion proteins (MFPs) such as Escherichia coli CvaA (UniProt Accession #P22519). In some embodiments, the MFP comprises an amino acid sequence as set forth in Table 7.

TABLE 7

| | ID/RefSeq | | | SEQ ID | |
|---|---|---|---|---|---|
| Group | Accession | Description | Taxon | NO | Sequence |
| 1 | E492_MFP_mceH | Membrane | Klebsiella | 191 | MKWRGRAILLPGIPLWLTMLGSI |
| | | fusion protein | pneumoniae | | AFITAFLTFVIAGTYSRRVNVSGE |
| | | | | | VTTWPRAVNIYSGVQGFVVRQF |
| | | | | | VHEGQSIKKGDPIYQIDVSKSTRS |
| | | | | | GVVTDNQRRDIENQLVRVDNIIS |
| | | | | | RLEESKKITLNTLEKQHLQYSDA |
| | | | | | FRRSSDIIRRAEEGIKIMKNNMEN |
| | | | | | YRNYQLKGLINKDQLTNQVALY |
| | | | | | YQQQNNLLSLSGQNEQNALQITS |
| | | | | | LESQIQTQAADFDNRIYQMELQR |
| | | | | | YELQKELIDTDVGGEIIIRALSDG |
| | | | | | KVDSLSVTVGQMVNAGDSLLQV |
| | | | | | IPENIENYYLILWVPNDAVPYISA |
| | | | | | GDKVNIRYEAFPAEKFGQFSATV |
| | | | | | KTISRTPASTQEMLTYKGAPQNT |
| | | | | | LGTSVPWYKVIVKPEKQIISYGG |
| | | | | | KSLPLENGMKAESTLFLEKRRIY |
| | | | | | QWMLSPFYDMKHSATGPIDE |
| 1 | WP_024913115.1 | HlyD family | Chania | 192 | MFRPEAILSHRMQWRGQALLLP |
| | | secretion | multi- | | GIPFWAMAGSCLFFIATFLTFIIA |
| | | protein | tudinisentens | | GTYTRRVNVVGEISTYPRAANV |
| | | | | | YSSVQGVVTKQFVTEGQMIKAE |
| | | | | | EPIYQIDVSKSTRSGVISDNQRRD |
| | | | | | IGNQLTRIAQIIIRLENSKKATLN |
| | | | | | MLEAQKAQYSTAFQHSTDILQR |
| | | | | | AQEGIRIMKENMENYRRYQSKG |
| | | | | | LINKDQLTNQIASYYQLQNNLLG |
| | | | | | LSSQNEQNALQINALGSQIQTQA |
| | | | | | AEFDNQIYRMEMQRYELQKELL |
| | | | | | NIDADGEIIVRALADGRVDSLSV |
| | | | | | TVGQMVNVGDSLLQLIPHNIDH |
| | | | | | YSLVLWVPNDAIPYVSIGNRVNI |
| | | | | | RYEAFPAEKFGQFVGTVAVISKT |
| | | | | | PASPQEMLTYQGAPRAAQTTAV |
| | | | | | PYYKVIVSPKKQTIVYNAQRLNL |
| | | | | | QNGMKAQSTLFLEKRKIYQWML |
| | | | | | SPFYDMKHSVMGPVNE |
| 1 | WP_014227600.1 | MULTISPECIES: | Klebsiella | 193 | MLKNIYRKEAIEYKKIHWKGKA |
| | | HlyD | | | LLLAGIPAWLVTLLASLFLIALVL |
| | | family | | | SLIFCTFTQRIDVRGEVITLPHSV |
| | | secretion | | | NVFAPQQGFVVNQHVKVGDIVN |
| | | protein | | | KGQPLYELDVSRNTINGNVSAA |
| | | | | | QIEVINEKIANAEDIISKLMRNKS |
| | | | | | ETLTALEKQIKTTSASLAETNRM |
| | | | | | LATTQVGLNKMFTNLSSYDKYL |
| | | | | | KDGLITKDQYNYQHSLYFQQQS |
| | | | | | AYQSLVTQKMQLESQLTQTNSD |
| | | | | | KITKAADFDNQISSQHNQINDYK |
| | | | | | NQLVESNANGNLIIKATTNGKIE |
| | | | | | SLAVTKGQMVENGSSLAQIKPT |
| | | | | | GDIEYYLILWLPNNTIPYVKPGD |
| | | | | | TINIRYDAFPADKFGQFPGKVISI |
| | | | | | SSVPASRQEMAEYTNVNNGTSQ |
| | | | | | QELALYKAIIKIKDKTFSYNGKT |
| | | | | | LTLSNGLKAQAVVFLEERPLYM |
| | | | | | WMFTPFYKMTQSVSGPIDE |
| 1 | WP_107331972.1 | HlyD family | Klebsiella | 194 | MTCLFAPSKALLLAGLPAWLVT |
| | | secretion | pneumoniae | | CLSLLFLCALICALIFCKFTQRID |
| | | protein | | | VKGEVITLPHSVNVFSPQQGFVV |
| | | | | | NQYVQIGDVVKKGQTLYELDVS |
| | | | | | RNTTTGNVSAAQIEVINEKIANSE |
| | | | | | AIIKKLTHNKNETLIALDAQLKN |
| | | | | | ARNSLNETVRMLANTQQGLSKM |
| | | | | | HENLSSYDKYLKEGLITKDQYN |
| | | | | | YQHSLYFQQQSAYQSLISQKMQ |
| | | | | | LETQLTQLSSDKVTKAADFDNQI |
| | | | | | SSQYNQTNDYKNQLVESNANGN |
| | | | | | IIIKATTEGRIESLAVTKGQMVDK |
| | | | | | GSSLAQIKPIGNIEYYLILWLPNN |
| | | | | | SIPYVKVGDTINIRYDAFPSDKFG |
| | | | | | QFPGEIISISSLPASRQEMSEYTNV |
| | | | | | NDGTNQQELALYKAIVKIRDKK |
| | | | | | FNYDGKELSLSNGLKAQAVVFL |
| | | | | | EERPLYMWMFTPVYKISQSVSGP |
| | | | | | VND |
| 1 | WP_006250714.1 | HlyD family | Mannheimia | 195 | MKSQIQATEQAITALYKNKSETI |
| | | efflux | haemolytica | | NSLEKQIKNSQKIYQDKQSYLVE |
| | | transporter | | | IEKSMNDYAELVKRYEKLLKVG |
| | | periplasmic | | | HSSHDEVNIQKSRYFQQKSLFNE |
| | | adaptor | | | LKQELIQLKSTQLSLENEIETRKT |
| | | subunit | | | EFNNQIIRYEMQKSDLNIRLMEF |
| | | | | | ESVSDIIVNASLDGKIESTSVTVG |
| | | | | | QIIKENDPLAQILPQNKGNYQLV |
| | | | | | MWVPNSAISFIKQGDEVNIRYEA |
| | | | | | FPFEKFGQFKGKITSISTLPASLQE |
| | | | | | LSFYKNLPVNLEQGIPLYKILIEL |
| | | | | | ADQNVKYNDTSLYFMSGMKAE |
| | | | | | ATLFLEKRKLYEWIFFPMYQLRK |
| | | | | | NMEQ |
| 1 | WP_080762684.1 | HlyD family | Mannheimia | 196 | MIFGSYTRRETVFGELVMQAHPI |
| | | efflux | haemolytica | | ILSAPKSGYISENYIQPHQQVKKG |
| | | transporter | | | DPLFKITLDRITHSGNINVNSILSL |
| | | periplasmic | | | KSQIQATEQAITALNKNKLETINS |
| | | adaptor | | | LENQIKNNQKIYQDKQAYLVEV |
| | | subunit | | | EKTMNDYEDLVKRYEKLLKVG |
| | | | | | HSSYDEVNIQKSRYFQQKSLFNE |
| | | | | | LKQELIQLKSTQLSLENEIETRKT |
| | | | | | EFNNQIIRYEMQKSDLNIRLMEF |
| | | | | | ESVSDIIVNASLDGKIESTSVTVG |
| | | | | | QIIKENDPLAQILPQNKGNYQLV |
| | | | | | MWVPNSAISFIKQGDEVNIRYEA |
| | | | | | FPFEKFGQFKGKITSISTLPASLQE |
| | | | | | LSFYKNLPANLEQGIPLYKILIEL |
| | | | | | ADQNVKYNDTSLYFMSGMKAE |
| | | | | | ATLFLEKRKLYEWIFFPMYQLRK |
| | | | | | NMEQ |
| 1 | WP_023180562.1 | HlyD family | Salmonella | 197 | MFRQEALDNRKMKWRGRAILLP |
| | | secretion | enterica | | GMPLWLIMLGSIAFITAFLTFVIA |
| | | protein | | | GTYSRRVNVSGEVTTWPRAVNI |
| | | | | | YSGVQGFIVRQFVHEGQSIKKGD |
| | | | | | PIYQIDVSKSTRSGVVTDNQRRDI |
| | | | | | ENQLVRVDNIISRLAESKKITLNT |
| | | | | | LEKQRLQYSDAFRLSSDIIRQAA |
| | | | | | EGIKIMKNNMENYRNYQSKGLIT |
| | | | | | QDQLTNQVALYYQQQNNLLSLS |
| | | | | | GQNEQNALQITSLESQIQTQAAD |
| | | | | | FDNRIYQMELQRYELQKELVDT |
| | | | | | DVGGEIIIRALSDGKVDSLSVTV |
| | | | | | GQMVNAGDSLLQVIPENIENYYL |
| | | | | | IIWVPNDAVPYISAGDKVNIRYE |
| | | | | | AFPAEKFGQFSATVKTISRTPAST |
| | | | | | QEMLTYKGAPQNTLGISVPWYK |
| | | | | | VIVKPEKQIISYDGKSLPLENGM |
| | | | | | KAESTLFLEKRRIYQWMLSPFYD |
| | | | | | MKHSATGPIDE |
| 1 | WP_201264602.1 | HlyD family | Salmonella | 198 | MFRQEALDNRKMKWRGRAILLP |
| | | secretion | enterica | | GMPLWLTMLGSIAFITAFLTFVIA |
| | | protein | | | GTYSRRVNVSGEVTTWPRAVNI |
| | | | | | YSGVQGFIVRQFVHEGQSIKKGD |
| | | | | | PIYLIDVSKSTRSGVVTDNQRRDI |
| | | | | | ENQLVRVDNIISRLEESKKITLNT |
| | | | | | LEKQRLQYSDAFRLSSDIIRQAA |
| | | | | | EGIKIMKNNMENYRNYQSKGLIT |
| | | | | | KDQLTNQVALYYQQQNNLLSLS |
| | | | | | GQNEQNALQITSLESQIQTQAAD |
| | | | | | FDNRIYQMELQRYELQKELVDT |
| | | | | | DVGGEIIIRALSDGKVDSLSVTV |
| | | | | | GQMVNAGDSLLQVIPENIENYYL |
| | | | | | ILWVPNDAVPYISAGDKVNIRYE |
| | | | | | AFPAEKFGQFTATVKTISRTPAST |
| | | | | | QEMLTYKGAPQNTLGISVPWYK |
| | | | | | VIVKPEKQIISYDGKSLPLENGM |
| | | | | | KAESTLFLEKRRIYQWMLSPFYD |
| | | | | | MKHSATGPIDE |
| 1 | WP_065506759.1 | HlyD family | Serratia | 199 | MEQDDLFRREAVSHNRSKWAG |
| | | secretion | inhibens | | KALLIAGLPAWATAVISLSFIAVL |
| | | protein | | | VIFVTYGTYTRRISVGGEVTTLPR |
| | | | | | TVSIFATQQGFIAQRFVNVGEQV |
| | | | | | KKGQHLYQIDVSRATDSGTVSA |
| | | | | | NTRQAIENQLAQVDSIISKLEQN |
| | | | | | KKTTSDNLRAQIEQYKLSHDQSK |
| | | | | | KMVENSRNGVNFMRETMKNYN |
| | | | | | EYRNKGLINKDQLNNQVYLYYQ |
| | | | | | QQNTFQSLYSQSIQEALQITNLSS |
| | | | | | DLVTRASDFDNQISQYQFQKND |
| | | | | | LLRQLSEADASGSLIVNAPTNGR |
| | | | | | VESLSVTPGQMVNVGDSLTQLIP |
| | | | | | SDHAIYYLVLWLPNSSVPYVNSG |
| | | | | | DNINIRYDAFPFEKFGQFPGVIDN |
| | | | | | VAYVPVSNQEMSSYGSSPMRQN |
| | | | | | GEVVESYYKVLVAIDNTRFGYQ |
| | | | | | GKELNLSSGMKAQATLFLEKRPI |
| | | | | | YQWMFAPFYDMKNSVSGPIREQ |
| | | | | | NL |
| 2 | ER18_MFP_cvaA | Membrane | Mixta gaviniae | 200 | MSESLFRQEALEANKTSAIGNVA |
| | | fusion protein | | | LYCPPYRWLVISLVAAIAIAVAA |
| | | | | | FFTFGSYTKRETAIGQLMPAKGV |
| | | | | | MNVASMAAGTVIDIHVEEGQRV |
| | | | | | EKGEAIATVSSEVFTALGQTRET |
| | | | | | VAQQLRLQRERLRGDLDNQQKL |
| | | | | | FAEAMSGLQERERLLGSQLEQL |
| | | | | | DLQQTQRARQARLARRQLDKLN |
| | | | | | LMRKEGYASNSQVEQQEAAVID |
| | | | | | ADARLQDIARQRLDIHQQLAQT |
| | | | | | RQQLRELPMNTRNKQNEIERRLS |
| | | | | | ELEQSMAENESRRSIVLRAPMEG |
| | | | | | MVGSVMVKAGQMVSAGQTLFS |
| | | | | | VLPDEGRLQARIMVSSRAIGFIRP |
| | | | | | GQKVVLRYQAFPFQKFGQQYGK |
| | | | | | VAEVSRVALSPQEVATLTGNNN |
| | | | | | VQEQHYRVVVALEKQHINVYGR |
| | | | | | QEKLRPGSALEADFLIDTRRLYE |
| | | | | | WVLEPLYALGRRAGN |
| 2 | WP_000097731.1 | MULTISPECIES: | Acinetobacter | 201 | MSSNLFRKEALDAKQAKWTGTII |
| | | HlyD | | | LSRPFSFTYLTICALCIALVIIAFAI |
| | | family efflux | | | WGSYTKRSTVQGQLIPQSGLIQV |
| | | transporter | | | YTTQSGTILKKNVHEGQTVKKG |
| | | periplasmic | | | DILFTISTASYGEQGSIADALAKQ |
| | | adaptor | | | TQLKEQSIRNEITRMRFIHQDEKR |
| | | subunit | | | TINNQIGLLNGTLVKVENLIANQ |
| | | | | | RERVNLAKKNQQRYGNILHENA |
| | | | | | ISHEEFEARKIDYLDQLAQYESL |
| | | | | | QREKVSLEKQLTEQQISLSGLEN |
| | | | | | RQNNQIEQLERLLSSNTQELIEM |
| | | | | | QSRQHIAIQANSSGVIGTINAEVG |
| | | | | | QFVDLSKPLLTVLPENTALIAQL |
| | | | | | YVPSRAIGFVKEGDQVLLRYQA |
| | | | | | YPYQKFGHAKAQVLSVAKTALA |
| | | | | | SQDLQTIGIISPQEQLNNEPVYLV |
| | | | | | RAKLDKQMVKAYGNDMPLQVG |
| | | | | | MTLEGDIMHEQRKLYEWVLEPL |
| | | | | | FSITGKL |
| 2 | WP_005141039.1 | HlyD family | Acinetobacter | 202 | MSSNLFRKEALDAKQAKWTGTII |
| | | efflux | baumannii | | LSRPFSFTFLTICALCIALVIIAFAI |
| | | transporter | | | WGSYTKRSTVQGQLIPQSGLIQV |
| | | periplasmic | | | YTTQSGTILKKNVYEGQSVKTG |
| | | adaptor | | | DILFTISTESYGEQGSITDALAKQ |
| | | subunit | | | TQLKEQSIRNEISRIRFIHQDEKRT |
| | | | | | INNQISLLNGTLVKVENLIYNQLE |
| | | | | | RINLAKRNQQRYEIMLKQDAAS |
| | | | | | YEEFEARKINYLDQLAQYESLQR |
| | | | | | EKVSLEKQLTEQQISLSGLENRQ |
| | | | | | NNQIEQLERLLSSNTQELIEMQSR |
| | | | | | QHIAIQANSSGLIGTINAEVGQYV |
| | | | | | DLSKPLLTVLPENTPLIAQLYVPS |
| | | | | | RAIGFVKEGDQVLLRYQAYPYQ |
| | | | | | KFGHAKAQVLSVAKTALASQDL |
| | | | | | QTIGIISPQEQLNNEPVYLVRAKL |
| | | | | | DKQIVKAYGKDMPLQVGMTLE |
| | | | | | GDIMHEKRKLYEWVLEPLFSITG |
| | | | | | KL |
| 2 | WP_104949567.1 | HlyD family | Enterobacter | 203 | MSETLFRQQAIDNNRVKSLGSVI |
| | | efflux | sp. SGAir0187 | | LFTPPYRWLLVVFVGMIGAAIVT |
| | | transporter | | | LLIFGSYTKRESARGTLVPEQGIL |
| | | periplasmic | | | DIVPVGSGTLTDIFIHEGQRVKK |
| | | adaptor | | | GEKLAAISSDISTAMGATHKQIA |
| | | subunit | | | QQLNEQREVLKSELVNLDAINSS |
| | | | | | TMQGIHDRIGLLKEQIAQLQLIN |
| | | | | | NQRRSQIGIEQEKLKNLRSLLAE |
| | | | | | GYASNTQIDQQESVRLEAVVRM |
| | | | | | QDVGRQLLELKQQLAQLEQQYI |
| | | | | | EQPINYLKQKNEVNQKIAELNQT |
| | | | | | MLENESRRGTILTAPADSIVGSV |
| | | | | | LVKKGQIVTSGQTIASLIPSDGNL |
| | | | | | QARVMISSRAIGFIKPGQKVVLR |
| | | | | | YDSFPYQKFGQQYGSVLEVSESA |
| | | | | | LTPKDVASVTGDNLAQEQFYQV |
| | | | | | KIVLKKQSISAYGIEHRLPPGSAV |
| | | | | | NADFIVDKRHLYQWVLEPLYAL |
| | | | | | GKRF |
| 2 | WP_157953003.1 | HlyD family | Limnobaculum | 204 | MTDKKVKEGEFVKKGDELYVIS |
| | | efflux | parvum | | SERESQNSHGTQQVISAQIQSRL |
| | | transporter | | | ASLNEDLTKNQTQFTSEKSLLQS |
| | | periplasmic | | | YIRQLNDQVNTLDKQIVNQRKIL |
| | | adaptor | | | QLVQRRQLQYQEIYKKEYISLEQ |
| | | subunit | | | FERVKEELLQQQSALSSYEREKI |
| | | | | | NAQKELSGRQSELDGLLLKFDK |
| | | | | | QQAQIDRNISTTRQELAESEAKR |
| | | | | | EITIQAPQTGTITAAVAQAGQFV |
| | | | | | DSSKPIVSIVPENSLLMVHLYAPS |
| | | | | | NAVGFIQEGAEVWLRYQAYPYQ |
| | | | | | KFGQYRGKVVSISKAALLQHEL |
| | | | | | QSTMNVGNEASLYQIVVKPDKQ |
| | | | | | RISVYGKDKALQPGMELEADITL |
| | | | | | DKRRLYEWVLEPLLSITKKATD |
| 2 | WP_061060995.1 | HlyD family | Pantoea vagans | 205 | MEQSLFRQEALDAANRGNLGIV |
| | | efflux | | | ALYCPPYRWLVISVVVFITAVTA |
| | | transporter | | | LFFIFGSYTKYESSTGELLPENGM |
| | | periplasmic | | | LIVPPPVSATVVDIPVKEGQLVK |
| | | adaptor | | | KDDVLMVLSSEVSTQMGQTRQV |
| | | subunit | | | IAENLVAQRERLQQDLQTLAKL |
| | | | | | HDVEMKGLSDTIASLKLQQEQL |
| | | | | | RLQLSHRRKQVALAKLQLDKLN |
| | | | | | AMHLEGYASNRQLEEQESNLLD |
| | | | | | SQARYQEYQRQLLDTSQKIVQA |
| | | | | | EQQLHEKPLDDEKKRNDIERQL |
| | | | | | ADNRQSMAENEARRSIELRAPKS |
| | | | | | GYVGMIMVKNGQMLNAGQSAI |
| | | | | | AILPNNTNLVARIMVNTQSIGFIQ |
| | | | | | PGQRVVLRYKAFPYQKFGQQYG |
| | | | | | KVIEVSRTALSPQEVTTLTGKNN |
| | | | | | VQEQQYRVLVSLDKQTISAYSQ |
| | | | | | NEKLKPGMALDADFIVDKRRLY |
| | | | | | EWVLEPIFALGHKISL |
| 2 | WP_003093492.1 | MULTISPECIES: | Pseudomonas | 206 | MFRQEALDAQHAGGLGEIVLIRP |
| | | HlyD | | | VSFTFLTLLAAAMALLVVGFFLF |
| | | family efflux | | | GSYTKRSTVSGQLVPASGQVKV |
| | | transporter | | | HAPQAGIVLRKFVQEGQAVRRG |
| | | periplasmic | | | ERLMVLSSERYGSDAGPVQAGIS |
| | | adaptor | | | RRLEQRRDSLRDELEKLRRLQD |
| | | subunit | | | DERDSLTSKVASLQRELTTLAAQ |
| | | | | | TDSQQRLLALASDAAARYQGLM |
| | | | | | DKGYISMDQLQQRQAELLGQRQ |
| | | | | | TLQGLERERTSLRQQLTERRNEL |
| | | | | | AGLSARQANQLAETRRQLSAVE |
| | | | | | QDLAESEAKRTLLVTAPESGIAT |
| | | | | | AVLAEAGQTVDSSRPLLSIVPAD |
| | | | | | TPLQAELYAPSKSIGFIRPGDAVL |
| | | | | | IRYQAYPYQKFGQYHGKVQSISR |
| | | | | | ASVSYAELSSMVGGVPGLGQDG |
| | | | | | EQLYRLRVTLDDQAVTAYGQPR |
| | | | | | PLQSGMLLDADILQDTRRLYEW |
| | | | | | VLEPLYSLTGKL |
| 2 | WP_003118189.1 | MULTISPECIES: | Pseudomonas | 207 | MFRQEALDAQHAGGLGEIVLIRP |
| | | HlyD | | | VSFTFLTLLAAAMALLVVGFFLF |
| | | family efflux | | | GSYTKRSTVSGQLVPANGQVKV |
| | | transporter | | | HAPQAGIVLRKFVQEGQAVRRG |
| | | periplasmic | | | ERLMVLSSERYGSDAGPVQAGIS |
| | | adaptor | | | RRLEQRRDSLRDELEKLRRLQD |
| | | subunit | | | DERDSLTSKVASLQRELTTLAAQ |
| | | | | | TDSQQRLLALASDAAARYQGLM |
| | | | | | DKGYISMDQLQQRQAELLGQRQ |
| | | | | | TLQGLERERTSLRQQLTERRNEL |
| | | | | | AGLSARQANQLAETRRQLSAVE |
| | | | | | QDLAESEAKRTLLVTAPESGIAT |
| | | | | | AVLAEAGQTVDSSRPLLSIVPAD |
| | | | | | TPLQAELYAPSKSIGFIRPGDAVL |
| | | | | | IRYQAYPYQKFGQYHGKVQSISR |
| | | | | | ASVSYAELSSMVGGVPGLGQDG |
| | | | | | EQLYRLRVTLDDQAVTAYGQPR |
| | | | | | PLQSGMLLDADILQDTRRLYEW |
| | | | | | VLEPLYSLTGKL |
| 2 | WP_003137547.1 | MULTISPECIES: | Pseudomonas | 208 | MFRQEALDAQHAGALGEIVLIRP |
| | | HlyD | | | VSFTFLTLLAAAMALLVVGFFLF |
| | | family efflux | | | GSYTKRSTVSGQLVPASGQVKV |
| | | transporter | | | HAPQAGIVLRKFVQEGQAVRRG |
| | | periplasmic | | | ERLMVLSSERYGSDAGPVQAGIS |
| | | adaptor | | | RRLEQRRDSLRDELEKLRRLQD |
| | | subunit | | | DERDSLTSKVASLQRELTTLAAQ |
| | | | | | TDSQQRLLALASDAAARYQGLM |
| | | | | | DKGYISMDQLQQRQAELLGQRQ |
| | | | | | TLQGLERERTSLRQQLTERRNEL |
| | | | | | AGLSARQANQLAETRRQLSAVE |
| | | | | | QDLAESEAKRTLLVTAPESGIAT |
| | | | | | AVLAEAGQTVDSSRPLLSIVPAD |
| | | | | | TPLQAELYAPSKSIGFIRPGDAVL |
| | | | | | IRYQAYPYQKFGQYHGKVQSISR |
| | | | | | ASVSYAELSSMVGGVPGLGQDG |
| | | | | | EQLYRLRVTLDDQAVTAYGQPR |
| | | | | | PLQSGMLLDADILQDTRRLYEW |
| | | | | | VLEPLYSLTGKL |
| 2 | WP_003371197.1 | MULTISPECIES: | Pseudomonas | 209 | MLFRPEALAAQKRSTLGTVLLIS |
| | | HlyD | | | PVSMRLAAAVALLFCICMGLFLT |
| | | family efflux | | | FATYTKRTAASGVVLPEKGLIRI |
| | | transporter | | | YAPQAGVVNNLGISEGQPVNAD |
| | | periplasmic | | | AVLMALSSDTQNGEAGGAQRAI |
| | | adaptor | | | SLSIKRRQESLTQEIDKTLALHQQ |
| | | subunit | | | ELEGKKRQVEALETEQQKILAQI |
| | | | | | DLARQRLALTKGIAERYANLQR |
| | | | | | QDYVSRDQLQEKEDAVLDMRL |
| | | | | | RGEELNRSLLTVRAESTRLRAEL |
| | | | | | VELPFNHSKQLADLQRRLAENQ |
| | | | | | DQLVESETKREVLITAPTAGEAT |
| | | | | | AIAVSNGSRVDSARPLLSIVPAN |
| | | | | | AKLHAELYLPSRSVGFVRAGDT |
| | | | | | VMLRYQAYPYQRFGLQPGRVSS |
| | | | | | ISRTALPAEEVMTLGNVSEQMRE |
| | | | | | QGPFYRVTVALASQVIEGQGKR |
| | | | | | ERLRSGMQLDADIMQEKLPLYE |
| | | | | | WLLEPLRGIGKRL |
| 2 | WP_004351723.1 | MULTISPECIES: | Pseudomonas | 210 | MFRQEALDAQHAGGLGEIVLIRP |
| | | HlyD | | | VSFTFLTLLAAAMALLVVGFFLF |
| | | family efflux | | | GSYTKRSTVSGQLVPASGQVKV |
| | | transporter | | | HAPQAGIVLRKFVQEGQAVRRG |
| | | periplasmic | | | ERLMVLSSERYGSDAGPVQAGIS |
| | | adaptor | | | RRLEQRRDSLRDELEKLRRLQD |
| | | subunit | | | DERDSLTSKVASLQRELTTLAAQ |
| | | | | | TDSQRRLLALASDAAARYQGLM |
| | | | | | DKGYISMDQLQQRQAELLGQRQ |
| | | | | | TLQGLERERTSLRQQLTERRNEL |
| | | | | | AGLSARQANQLAETRRQLSAVE |
| | | | | | QDLAESEAKRTLLVTAPESGIAT |
| | | | | | AVLAEAGQTVDSSRPLLSIVPAD |
| | | | | | TPLQAELYAPSKSIGFIRPGDAVL |
| | | | | | IRYQAYPYQKFGQYHGKVQSISR |
| | | | | | ASVSYAELSSMVGGVPGLGQDG |
| | | | | | EQLYRLRVTLDDQAVTAYGQPR |
| | | | | | PLQSGMLLDADILQDTRRLYEW |
| | | | | | VLEPLYSLTGKL |
| 2 | WP_023094006.1 | MULTISPECIES: | Pseudomonas | 211 | MYQALGKVTLTRPVSFTFLTLLA |
| | | HlyD | | | AAMALLVVGFFLFGSYTKRSTV |
| | | family efflux | | | SGQLVPASGQVKVHAPQAGIVL |
| | | transporter | | | RKFVQEGQAVRRGERLMVLSSE |
| | | periplasmic | | | RYGSDAGPVQAGISRRLEQRRDS |
| | | adaptor | | | LRDELEKLRRLQDDERDSLTSKV |
| | | subunit | | | ASLQRELTTLAAQTDSQQRLLAL |
| | | | | | ASDAAARYQGLMDKGYISMDQ |
| | | | | | LQQRQAELLGQRQTLQGLERER |
| | | | | | TSLRQQLTERRNELAGLSARQA |
| | | | | | NQLAETRRQLSAVEQDLAESEA |
| | | | | | KRTLLVTAPESGIATAVLAEAGQ |
| | | | | | TVDSSRPLLSIVPADTPLQAELYA |
| | | | | | PSKSIGFIRPGDAVLIRYQAYPYQ |
| | | | | | KFGQYHGKVQSISRASVSYAELS |
| | | | | | SMVGGVPGLGQDGEQLYRLRVT |
| | | | | | LDDQAVTAYGQPRPLQSGMLLD |
| | | | | | ADILQDTRRLYEWVLEPLYSLTG |
| | | | | | KL |
| 2 | WP_036996088.1 | MULTISPECIES: | Pseudomonas | 212 | MLFRSEAVHARRDKELGSVLLV |
| | | HlyD | | | APVPLRWAALAALLFSVLLIVFL |
| | | family efflux | | | TCASYTRRATVTGVLVPQGGLIK |
| | | transporter | | | VFAPQAGVVQGLAAVEGQTVEA |
| | | periplasmic | | | GATLMALGSDLYAEAGGAQVAI |
| | | adaptor | | | SRLIKRRNDSLAEEIATTRALHLQ |
| | | subunit | | | ELEGKRRRLAVLESEQHKVVTQ |
| | | | | | MDISRQRLGLAQGIAARYADLQ |
| | | | | | RQDYVSRDQLQEKQDAVLEVRL |
| | | | | | RSEELSRSLLSLRNDIATLRAELA |
| | | | | | ELPFNQGKQLAELERRLSQSQGS |
| | | | | | LLESEVKRQVVITAPASGEVTAI |
| | | | | | GVVNGTRADSNRPLLSIVPKDAQ |
| | | | | | LYAELYVPSRSVGFVRPGDAVLL |
| | | | | | RYQAYPYQHFGLARGTVASVSR |
| | | | | | SALPADEIMSVGGVSEQQREQGP |
| | | | | | LYRVRVTLEQQSMLGRGLNERL |
| | | | | | RSGMQLDADILQENMPLYEWLL |
| | | | | | EPLRGIGKRL |
| 2 | WP_054073231.1 | MULTISPECIES: | Pseudomonas | 213 | MLFRPEALDAQKRSTLGTVLLVS |
| | | HlyD | | | PLSMRLAAAVALLFCISMGLFLT |
| | | family efflux | | | FATYTKRTAASGILLPETGLIRIY |
| | | transporter | | | SPQAGVITRLDIVEGQHVIDGAV |
| | | periplasmic | | | LMALSSDTQSGEAGGAQRAISLS |
| | | adaptor | | | IKRRQESLAQEIDETLALHLQELE |
| | | subunit | | | GKKRQVAALETEQQKILAQIDLT |
| | | | | | RQRLALTKGLAERYANLQRQDY |
| | | | | | VSRDQLQEKQDAVLDMRLRGEE |
| | | | | | LNRSLLTVRAESTRLRAELVELP |
| | | | | | FNQSKQLADLRRRLAENQDRLIE |
| | | | | | SETKREIFITSPTLGEATAIAVSNG |
| | | | | | SRVDSTRPLLSIVPADAKLHAEL |
| | | | | | YLPSRSVGFVRTGDTVMLRYQA |
| | | | | | YPYQRFGLQSGRVSSISRTALPA |
| | | | | | DEVMTLGNVSEPMREQGPFYRV |
| | | | | | TVALASQTIDGQGMHERLRSGM |
| | | | | | QLDADIMQERLPLYEWMLEPLR |
| | | | | | GIGKRL |
| 2 | WP_003111248.1 | HlyD family | Pseudomonas | 214 | MFRQEALDAQHAGGLGEIVLIRP |
| | | efflux | aeruginosa | | VSFTFLTLLAAAMALLVVGFFLF |
| | | transporter | | | GSYTKRSTVSGQLVPASGQVKV |
| | | periplasmic | | | HAPQAGIVLRKFVQEGQAVRRG |
| | | adaptor | | | ERLMVLSSERYGSDAGPVQAGIS |
| | | subunit | | | RRLEQRRDSLRDELEKLRRLQD |
| | | | | | DERDSLTSKVASLQRELTTLAAQ |
| | | | | | TDSQQRLLALASDAAARYQGLM |
| | | | | | DKGYISMDQLQQRQAELLGQRQ |
| | | | | | TLQGLERERTSLRQQLTERRNEL |
| | | | | | AGLSARQANQLAETRRQLSTVE |
| | | | | | QDLAESEAKRTLLVTAPESGIAT |
| | | | | | AVLAEAGQTVDSSRPLLSIVPAD |
| | | | | | TPLQAELYAPSKSIGFIRPGDAVL |
| | | | | | IRYQAYPYQKFGQYHGKVQSISR |
| | | | | | ASVSYAELSSMVGGVPGLGQDG |
| | | | | | EQLYRLRVTLDDQAVTAYGQPR |
| | | | | | PLQSGMLLDADILQDTRRLYEW |
| | | | | | VLEPLYSLTGKL |
| 2 | WP_012613567.1 | HlyD family | Pseudomonas | 215 | MFRQEALDAQHAGGLGEIVLIRP |
| | | efflux | aeruginosa | | VSFTFLTLLAAVMALLVVGFFLF |
| | | transporter | | | GSYTKRSTVSGQLVPASGQVKV |
| | | periplasmic | | | HAPQAGIVLRKFVQEGQAVRRG |
| | | adaptor | | | ERLMVLSSERYGSDAGPVQAGIS |
| | | subunit | | | RRLEQRRDSLRDELEKLRRLQD |
| | | | | | DERDSLTSKVASLQRELTTLAAQ |
| | | | | | TDSQRRLLALASDAAARYQGLM |
| | | | | | DKGYISMDQLQQRQAELLGQRQ |
| | | | | | TLQGLERERTSLRQQLTERRNEL |
| | | | | | AGLSARQANQLAETRRQLSAVE |
| | | | | | QDLAESEAKRTLLVTAPESGIAT |
| | | | | | AVLAEAGQTVDSSRPLLSIVPAD |
| | | | | | TPLQAELYAPSKSIGFIRPGDAVL |
| | | | | | IRYQAYPYQKFGQYHGKVQSISR |
| | | | | | ASVSYAELSSMVGGVPGLGQDG |
| | | | | | EQLYRLRVTLDDQAVTAYGQPR |
| | | | | | PLQSGMLLDADILQDTRRLYEW |
| | | | | | VLEPLYSLTGKL |
| 2 | WP_016252981.1 | HlyD family | Pseudomonas | 216 | MFRQEALDAQHAGGLGEIVLIRP |
| | | efflux | aeruginosa | | VSFTFLTLLAAAMALLVVGFFLF |
| | | transporter | | | GSYTKRSTVSGQLVPASGQVKV |
| | | periplasmic | | | HAPQAGIVLRKFVQEGQAVRRG |
| | | adaptor | | | ERLMVLSSERYGSDAGPVQAGIS |
| | | subunit | | | RRLEQRRDSLRDELEKLRRLQD |
| | | | | | DERDSLTSKVASLQRELTTLAAQ |
| | | | | | TDSQRHLLALASDAAARYQGLM |
| | | | | | DKGYISMDQLQQRQAELLGQRQ |
| | | | | | TLQGLERERTSLRQQLTERRNEL |
| | | | | | AGLSARQANQLAETRRQLSAVE |
| | | | | | QDLAESEAKRTLLVTAPESGIAT |
| | | | | | AVLAEAGQTVDSSRPLLSIVPAD |
| | | | | | TPLQAELYAPSKSIGFIRPGDAVL |
| | | | | | IRYQAYPYQKFGQYHGKVQSISR |
| | | | | | ASVSYAELSSMVGGVPGLGQDG |
| | | | | | EQLYRLRVTLDDQAVTAYGQPR |
| | | | | | PLQSGMLLDADILQDTRRLYEW |
| | | | | | VLEPLYSLTGKL |
| 2 | WP_023123651.1 | HlyD family | Pseudomonas | 217 | MYQALGKVTLTRPVSFTFLTLLA |
| | | efflux | aeruginosa | | AVMALLVVGFFLFGSYTKRSTV |
| | | transporter | | | SGQLVPASGQVKVHAPQAGIVL |
| | | periplasmic | | | RKFVQEGQAVRRGERLMVLSSE |
| | | adaptor | | | RYGSDAGPVQAGISRRLEQRRDS |
| | | subunit | | | LRDELEKLRRLQDDERDSLTSKV |
| | | | | | ASLQRELTTLAAQTDSQRRLLAL |
| | | | | | ASDAAARYQGLMDKGYISMDQ |
| | | | | | LQQRQAELLGQRQTLQGLERER |
| | | | | | TSLRQQLTERRNELAGLSARQA |
| | | | | | NQLAETRRQLSAVEQDLAESEA |
| | | | | | KRTLLVTAPESGIATAVLAEAGQ |
| | | | | | TVDSSRPLLSIVPADTPLQAELYA |
| | | | | | PSKSIGFIRPGDAVLIRYQAYPYQ |
| | | | | | KFGQYHGKVQSISRASVSYAELS |
| | | | | | SMVGGVPGLGQDGEQLYRLRVT |
| | | | | | LDDQAVTAYGQPRPLQSGMLLD |
| | | | | | ADILQDTRRLYEWVLEPLYSLTG |
| | | | | | KL |
| 2 | WP_069382813.1 | HlyD family | Pseudomonas | 218 | MFRQEALDAQHAGGLGEIVLIRP |
| | | efflux | aeruginosa | | VSFTFLTLLAAAMALLVVGFFLF |
| | | transporter | | | GSYTKRSTVSGQLVPASGQVKV |
| | | periplasmic | | | HAPQAGIVLRKFVQEGQAVRRG |
| | | adaptor | | | ERLMVLSSERYGSDAGPVQAGIS |
| | | subunit | | | RRLEQRRDSLRDELEKLRRLQD |
| | | | | | DERDSLTSKVASLQRELTTLAAQ |
| | | | | | TDSQQRLLALASDAAARYQGLM |
| | | | | | DKGYISMDQLQQRQAELLGQRQ |
| | | | | | TLQGLERERTSLRQQLTERRNEL |
| | | | | | AGLSARQANQLAETRRQLSAVE |
| | | | | | QDLAESEAKRTLLVTAPESGIAT |
| | | | | | AVLAEAGQTVDSSRPLLSIVPAD |
| | | | | | TPLQAELYAPSKSIGFIRPGDAVL |
| | | | | | IRYQAYPYQKFGQYHGKVQSISR |
| | | | | | ASVSYAELSSMVGGVPGLGQDG |
| | | | | | EQLYRLRVTLDDQAVTAYGQPR |
| | | | | | PLQSGMLLDADILQDTRRLYEW |
| | | | | | LLEPLYSLTGKL |
| 2 | WP_079741046.1 | HlyD family | Pseudomonas | 219 | MFRQEALDAQHAGSLGRIVLIRP |
| | | efflux | aeruginosa | | VSFTFLTLLAAAMALLVVGFFLF |
| | | transporter | | | GSYTKRSTVSGQLVPASGQVKV |
| | | periplasmic | | | HAPQAGIVLRKFVEEGQAVRRG |
| | | adaptor | | | ERLMVLSSERYGSDAGPVQAGIS |
| | | subunit | | | RQLEQRRDSLRDELEKLRRLQD |
| | | | | | DERASLASKVASLQRELATLAA |
| | | | | | QTDSQQRLLALASDAAARYQGL |
| | | | | | MDKGYISMDQLQQRQAELLGQR |
| | | | | | QTLQGLARERTSLEQQLTERRNE |
| | | | | | LAGLSARQANQLAETRRQLSAV |
| | | | | | QQDLAESEAKRTLLVTAPESGIA |
| | | | | | TAVLAEAGQTVDSSRPLLSIVPA |
| | | | | | DTPLQAELYAPSKSIGFIRPGDAV |
| | | | | | LIRYQAYPYQKFGQYHGKVRSIS |
| | | | | | RASVSYAELSSMVGGVPGLGQD |
| | | | | | GEQLYRLRVTLDDQAVTAYGQP |
| | | | | | RPLQSGMLLEADILQDTRRLYE |
| | | | | | WVLEPLYSLTGKL |
| 2 | WP_005745444.1 | HlyD family | Pseudomonas | 220 | MLFRPEALDAQKRSTLGTVLLVS |
| | | efflux | amygdali | | PLSMRLAAAVALLFCISMGLFLT |
| | | transporter | | | FATYTKRTAASGVLLPETGLIRIY |
| | | periplasmic | | | SPQAGVITRLDIVEGQHVTDGAV |
| | | adaptor | | | LMALSSDTQSGEAGGAQRAISLS |
| | | subunit | | | IKRRQESLAQEIDETLALHLQELE |
| | | | | | GKKRQVAALETEQQKILAQIDLT |
| | | | | | RQRLALTKGLAERYANLQRQDY |
| | | | | | VSRDQLQEKQDAVLDMRLRGEE |
| | | | | | LNRSLLTVRAESTRLRAELVELP |
| | | | | | FNQSKQLADLRRRLAENQDRLIE |
| | | | | | SETKREIFITAPTLGEATAIAVSN |
| | | | | | GSRVDSTRPLLSIVPADAKLHAE |
| | | | | | LYLPSRSVGFVRTGDTVMLRYQ |
| | | | | | AYPYQRFGLQPGRVSSISRTALP |
| | | | | | ADEVMTLGNVSEPMREQGPFYR |
| | | | | | VTVALASQAIDGQGMHERLRSG |
| | | | | | MQLDADIMQERLPLYEWMLEPL |
| | | | | | RGIGKRL |
| 2 | WP_062574733.1 | MULTISPECIES: | Pseudomonas | 221 | MFRSEALAARQSTWLGDIVLVR |
| | | HlyD | putida group | | PVSFTVMTVVALLLALMVVSFFF |
| | | family efflux | | | YGSYTRRSTVPGQLVPSSGQLKI |
| | | transporter | | | HSSQYGVVLERYVEEGQQVEQG |
| | | periplasmic | | | GRLFLISSERSVDSGPVQAEVSD |
| | | adaptor | | | QLQGQRRSLEEELRKQQQLQVE |
| | | subunit | | | ARQSLDSKLRSLTQELDTLAQQI |
| | | | | | ASQQRLVQLAGNAAERYQGLM |
| | | | | | DKGYISMDQLQQRQAELLGQRQ |
| | | | | | SLQGLVRESTVLRQQLVERQHE |
| | | | | | RAGLEALHGNQLASIRRSLSSVQ |
| | | | | | QALIESEAKRSLVITAPQPGVAT |
| | | | | | AILVGPGQVVDSSRALMSLVPA |
| | | | | | DANLQAELYAPSKAIGFIQAGDA |
| | | | | | VLLRYQAYPYQKFGQHHGQVIS |
| | | | | | VSRTTLSAAELANVVGSVPGLG |
| | | | | | GNGEQIYRIRVAIDRQSVQAYGE |
| | | | | | SRALQAGMLVEADVLQETRRLY |
| | | | | | EWVLEPLYSLTGKL |
| 2 | WP_042913837.1 | HlyD family | Pseudomonas | 222 | MLFRPEALAAQKRSTLGTVLLVS |
| | | efflux | syringae group | | PVSMRLAAAVALLFCMCMGLFL |
| | | transporter | genomosp. 3 | | SFATYTKRTAASGVLLPETGLIRI |
| | | periplasmic | | | YAPQTGVVNNLSIAEGQPVNAD |
| | | adaptor | | | AVLMALSSDTQNGETGGAQRAI |
| | | subunit | | | SLSIKRRQESLTQEIDKTLALHQQ |
| | | | | | ELEGKKRQVEALETEQQKILAQI |
| | | | | | DLARQRLALTKGIAERYANLQR |
| | | | | | QDYVSRDQLQEKEDAVLDMRL |
| | | | | | RGEELNRSLLTVRAESTRLRAEL |
| | | | | | VELPFNQSKQLADLQRRLAENQ |
| | | | | | DQLVESETKREILITAPTAGEATA |
| | | | | | LSVSNGSRVDSARPLLSIVPADA |
| | | | | | KLHAELCLPSRSVGFVRAGDTV |
| | | | | | MLRYQAYPYQRFGLQPGTVSSIS |
| | | | | | RTALPADEVMTLGNVSEQMREQ |
| | | | | | GPFYRVTVALASQTIEGQGKRDR |
| | | | | | LRSGMQLDADIMQEKLPLYEWL |
| | | | | | LEPLRGIGKRL |
| 2 | WP_032686862.1 | MULTISPECIES: | Raoultella | 223 | MNHDLFRQESLDANKTKVLGPV |
| | | HlyD | | | ALYCPPFRWLIVAMVCALTIVLV |
| | | family efflux | | | GFCSLGSYTKRETAKGVLTPESG |
| | | transporter | | | IMTITALTAGTVTALPVREGAGV |
| | | periplasmic | | | KKGERIATVSSEISTARYGQTRE |
| | | adaptor | | | AIARQLEIQSQGLTQQLTNLEQR |
| | | subunit | | | NAEALKSLQERSSLLAQQTTELD |
| | | | | | TIYRQRQRQIALSQKQVDKMAA |
| | | | | | MRAEGYASNTQVEQQESDLLDA |
| | | | | | KVRLQDVARQRIEIRQQHAQTR |
| | | | | | QQLREQPLTYFQQKNDLQQKLS |
| | | | | | DITQSMMENESRRSVDLRAPEEG |
| | | | | | TVSAVLVKPGQIVSAGQTIAMLL |
| | | | | | PDNAHLQARILLSSRAIGFIHTGQ |
| | | | | | RVVLRYESFPWQKFGQHSGAVS |
| | | | | | EISTSPLSPQEIAGITGNTQIQEPL |
| | | | | | YQVKVTLDSQSVQAYGKQIGLR |
| | | | | | PGSGLDADFIVDKRRIYEWVLEP |
| | | | | | LNALGKMTSL |
| 2 | WP_006316839.1 | HlyD family | Serratia | 224 | MSDTLFRQEVLDANKSKLIGSVA |
| | | efflux | inhibens | | LYCPPYRWLVISIAGFITLSIVIFFI |
| | | transporter | | | FGSYTKREAAKGELLPLDGVMN |
| | | periplasmic | | | IVPMVAGTVIGIPPREGQLVKKG |
| | | adaptor | | | DPLVTISSEVSTAMGQTREKIAE |
| | | subunit | | | QLKLQQEALEMDLKSLEGLNRE |
| | | | | | AIRGMKERENLLASQMKQLDLQ |
| | | | | | YQQRVRQANLARRQMEKTQLM |
| | | | | | RAQGYASNVQVEQQETVMLDA |
| | | | | | DAKLQDIARQRIDIQQQLAQTKQ |
| | | | | | QLREQPLNMRNQHNDIERKLSEI |
| | | | | | NQSLTENESRRSFVLRAPDNGM |
| | | | | | VGTVLVKLGQMVNAGQTAISLL |
| | | | | | PEDGRLQARIMVSSRAIGFIRPGQ |
| | | | | | KVVLRYQAFPYQKFGQQYGKVT |
| | | | | | EVSRIALSPQEVSTLTGMGNVQE |
| | | | | | QHYRVLVELEKQDINVYGKYEK |
| | | | | | LRPGSAVEADFLIDRRRLYEWVL |
| | | | | | EPLYALGRNSSV |
| 2 | WP_004952386.1 | HlyD family | Serratia | 225 | MNNDLFRKESLEANKTKVLGSV |
| | | efflux | plymuthica | | ALYCPPFRWLIIAIICTLVLALVA |
| | | transporter | | | LFIFGSYTKRETANGMLVPEGG |
| | | periplasmic | | | MMNITAINSGMVIALPVSEGDSL |
| | | adaptor | | | KKNDRIATVSSEISTRYGQTREAI |
| | | subunit | | | ARQLDLQQQGLNNQLLNLEQLN |
| | | | | | GETLKSLQEKERLLQQQSVELDT |
| | | | | | IYRQRTQQIALANKQLGKMKAM |
| | | | | | RTQGYASNTQVEQQENVLLDAS |
| | | | | | VRLQDVARQRIDARQQLAQTRQ |
| | | | | | QLREQPITYYQQKNDIQQKLSHI |
| | | | | | TQSMVENESRRSVDLRAPEKGM |
| | | | | | VSAVLVKTGQIVSAGQTIALMLP |
| | | | | | DNAHLQARIMLSSRAIGFIHTGQ |
| | | | | | RVVLRYQSFPWQKFGQQYGTV |
| | | | | | MEISKSTLSPQEVSAITGDNQVQ |
| | | | | | EALYQVKVAIDHQTVQAYGKQI |
| | | | | | GLLPGSGLQADFIVDKRRIYEWV |
| | | | | | LEPLNALGKMTSL |
| 2 | WP_087786518.1 | HlyD family | Steno- | 226 | MGLFLVLGQYTRRETVTGQLVP |
| | | efflux | strophomona | | ASGLINITALSPGTVARLHVEDG |
| | | transporter | maltophilia | | QSIQADEVLMEISSDQDTVRLGA |
| | | periplasmic | | | TRASIDEQLAEKEGRYRADLVSQ |
| | | adaptor | | | DSVARHQREALVARESLIRRQLD |
| | | subunit | | | QVGEQVALQAEEVEGSQALLERI |
| | | | | | EPLGKGGYISAFEIQRQQAVVRA |
| | | | | | ARSRKAELQRQGLELQQQLAAA |
| | | | | | QESLAKLPLEDATRRNEIVRELA |
| | | | | | DVAQSQAQNDLQRGTLFRARQG |
| | | | | | GVVAALLVKTGQMVSAGQPMIT |
| | | | | | ILPGDAKLQAQLLIPSRAIGFIAP |
| | | | | | GNRVILRYEAFPYQKFGQQYGR |
| | | | | | VSIVSRSALTPAEHSALTGRLPPA |
| | | | | | DQGPLYRVDVELDRQYVSAYGI |
| | | | | | KESIRPGMALQADVLIERRRLIE |
| | | | | | WLFEPLFGIQRRVFEENSNG |
| 2 | WP_039574409.1 | MULTISPECIES: | Xanthomonas | 227 | MCLGSFTRHEAVNGALVPDRGL |
| | | HlyD | | | LTLTPLSAGIVSNAFVAEGASVR |
| | | family efflux | | | AGEPIVEISGEQDSTSLGDTQASV |
| | | transporter | | | ISQLEIKRGRLNADLQAQNQLYA |
| | | periplasmic | | | AQQRDLGRRVALLQAQMDNTA |
| | | adaptor | | | EQIRLQRQRADSALGLYEQWVS |
| | | subunit | | | AAEKGILTKVQLLQQQDIAIQNQ |
| | | | | | AQLKELQKHALDLRVEHSQLQS |
| | | | | | QLEQTPATLEAKRNEIARQIADV |
| | | | | | AQSLSETEARRSVVLRAPTDGIV |
| | | | | | TNLLVHAGQPVGAQQPLITLLPK |
| | | | | | DIKLRAELWVPSKAVGFVTRGD |
| | | | | | NVLLRYQAFPYQKFGRHAGRVV |
| | | | | | DVSRSAISSKEVSSLLGQQIDDAR |
| | | | | | YRVVVELESQHILANGRQESLRP |
| | | | | | GMALDADILLEKRRLIEWMFEP |
| | | | | | VYDMTSRLSDDSAGQRG |
| 2 | WP_080948733.1 | MULTISPECIES: | Xanthomonas | 228 | MEPCWYEQARGLAAHGDAAAS |
| | | HlyD | | | SRQAARAGVGGRQRMTTLFRRE |
| | | family efflux | | | AVDYRQRSRLGPVVLRQHGALR |
| | | transporter | | | WCVGLLMATVILLLAGFFRLGF |
| | | periplasmic | | | ARSETLYGAVVPAGGLIAVTTPQ |
| | | adaptor | | | SGVVVQVGAVQGQRVAAGQVL |
| | | subunit | | | FVLSAEHRDDRGRPTQRAAVVL |
| | | | | | AEQQRLAVEAMAQLRAQGRVQ |
| | | | | | QQAAARALAGLRDRLQQIDVEL |
| | | | | | DLLRHRQQLTQSIEQRYRTALTR |
| | | | | | GLVSQQFVDEKQADVLDQRAHT |
| | | | | | LELRRERMALADALAQAQAEVQ |
| | | | | | QLPSNLRQQLAVAGAGLQEDRR |
| | | | | | AAIEQAAASRWEVRAPRAGRVA |
| | | | | | LRPLQRGQAVAQGQRLADLLPT |
| | | | | | SMATEVVLYAPSRAAGLIGLGM |
| | | | | | PVQLRFDALPYQHYGQFAGQVV |
| | | | | | EIAVAPEPPRVDSTSVSEPLYRVR |
| | | | | | VRLAGDAALRAGHTAVLRPGM |
| | | | | | RVQGTLALEWRRFSQWAFEPLS |
| | | | | | SLHGTLR |
| 2 | WP_011408202.1 | HlyD family | Xanthomonas | 229 | MTLFREEVMQARRNSWLGGISL |
| | | efflux | oryzae | | AQPVRAWVLASAAGIVAASVVV |
| | | transporter | | | FLCMGTYAHRSTVTGQLVPIKGL |
| | | periplasmic | | | ATVMAPATGVVSRLDVSEGQPV |
| | | adaptor | | | KAGQMLAVVAVPRATLDSGDT |
| | | subunit | | | QAALQHQLRKRGEGVKSSEQAQ |
| | | | | | LQQLDAQEQGLRAQLANLRQEL |
| | | | | | MQVDAEIATRRNQIALANEVLQ |
| | | | | | RWRQLQDDKYVSVLQIKQQEST |
| | | | | | ALEYTSQMQALQRQATEMRRSA |
| | | | | | AQIEQQLRVLPGQRGGVQADYR |
| | | | | | RDAAAVEQEQVQTQVNGALVV |
| | | | | | TAPVAGVVATQMAKPGQAIQLG |
| | | | | | QPLLSVVPGDGRLEAELLVPSRA |
| | | | | | IGFIAPGDTVLLRYQAFPYQKFG |
| | | | | | HQQGRLSRISRSALSSSELGALIG |
| | | | | | NVQQGEPYYRVTVGLGRQSVM |
| | | | | | AYGKPEMLKPGMLLQADIIGER |
| | | | | | RKLIEWVFEPLLSLGR |
| 2 | WP_011409246.1 | HlyD family | Xanthomonas | 230 | MTRLFRREVMDAQQRSRLGPVV |
| | | efflux | oryzae | | LRQRGALGWCVGVLMATVILLL |
| | | transporter | | | VGFFCLGFARRQTLYGAVVPAD |
| | | periplasmic | | | GMIAITTPQSGVVANVGVVQGQ |
| | | adaptor | | | RVAAGQVLFVLAAEHRDDRGRP |
| | | subunit | | | SQQAAAVLAEQQRLTAEAMVQ |
| | | | | | LRAQGRLQQQAAARALAGLRN |
| | | | | | RLEQVDAELGVLRHRQQLTQSIE |
| | | | | | QRYRTALTRGLVSQQFVDEKQA |
| | | | | | DVLDQRAHALELQRERLTLADA |
| | | | | | LAQAQAELQQLPVSLRQQLALA |
| | | | | | GASLQADRRTAIEQAAASRWEV |
| | | | | | RAPRAGRVALRPLQRGQAVGQG |
| | | | | | QRLADLLPTSTATEVVLYAPSRA |
| | | | | | AGLIGPGIPVQLRFDALPYQHYG |
| | | | | | QFAGRVVEIAAVPEPPRADAPLA |
| | | | | | SEPLYRMRVRLAGDAALRAGHA |
| | | | | | AVLRPGMRVQGTLALEWRRFSQ |
| | | | | | WAFEPLSSLHGTLR |
| 2 | WP_012444310.1 | HlyD family | Xanthomonas | 231 | MTRLFRREVMDAQQRSRLGPVV |
| | | efflux | oryzae | | LRQRGALGWCVGVLMATVILLL |
| | | transporter | | | VGFFCLGFARRQTLYGAVVPAD |
| | | periplasmic | | | GMIAITTPQSGVVANVGVVQGQ |
| | | adaptor | | | RVAAGQVLFVLAAEHRDDRGRP |
| | | subunit | | | SQQAAAVLAEQQRLTAEAMVQ |
| | | | | | LRAQGRLQQQAAARALAGLRN |
| | | | | | RLEQVDAELGVLRHRQQLTQFIE |
| | | | | | QRYRTALTRGLVSQQFVDEKQA |
| | | | | | DVLDQRAHALELQRERLTLADA |
| | | | | | LAQAQAELQQLPVSLRQQLALA |
| | | | | | GASLQADRRTAIEQAAASRWEV |
| | | | | | RAPRAGRVALRPLQRGQAVGQG |
| | | | | | QRLADLLPTSTATEVVLYAPSRA |
| | | | | | AGLIGPGIPVQLRFDALPYQHYG |
| | | | | | QFAGRVVEIAAAPEPPRADAALA |
| | | | | | SEPLYRVRVRLAGDAALRAGHA |
| | | | | | AVLRPGMRVQGTLALEWRRFSQ |
| | | | | | WAFEPLSSLHGTLR |
| 2 | WP_012445310.1 | HlyD family | Xanthomonas | 232 | MTLFRQEVLQARRNSWLGGISL |
| | | efflux | oryzae | | AQPVRAWVLASAAGIVAASVVV |
| | | transporter | | | FLCMGTYAHRSTVTGQLVPIKGL |
| | | periplasmic | | | ATVMAPATGVVSRLDVSEGQPV |
| | | adaptor | | | KAGQMLAVVAVPRATLDSGDT |
| | | subunit | | | QAALQHQLRKRGEGLKSSEQAQ |
| | | | | | LQQLDAQEQGLRAQLANLRQEL |
| | | | | | MQVDAEIATRRNQIALANEVLQ |
| | | | | | RWRQLQDDKYVSVLQIKQQEST |
| | | | | | ALEYTSQMQALQRQATEMRRSA |
| | | | | | AQIEQQLRVLPGQRGGVQADYR |
| | | | | | RDAAAVEQEQVQTQVNGALVV |
| | | | | | TAPVAGVVATQMAKPGQAIQLG |
| | | | | | QPLLSVVPGDGRLEAELLVPSRA |
| | | | | | IGFIAPGDTVLLRYQAFPYQKFG |
| | | | | | HQQGLLSRISRSALSSSELGALIG |
| | | | | | NAQQGEPYYRVTVALARQSVM |
| | | | | | AYGKPEMLKPGMLLQADIIGER |
| | | | | | RKLIEWVFEPLLSLGR |
| 2 | WP_014502353.1 | HlyD family | Xanthomonas | 233 | MTRLFRREVMDAQQRSRLGPVV |
| | | efflux | oryzae | | LRQRGALGWCVGVLMATVILLL |
| | | transporter | | | VGFFCLGFARRQTLYGTVVPAD |
| | | periplasmic | | | GMIAITTPQSGVVANVGVVQGQ |
| | | adaptor | | | RVAAGQVLFVLVAEHRDDRGRP |
| | | subunit | | | SQQAAAVLAEQQRLTAEAMDQ |
| | | | | | LRAQGRLQQQAAARALAGLRN |
| | | | | | RLEQVDAELGVLRHRQQLTQSIE |
| | | | | | QRYRTALTRGLVSQQFVDEKQA |
| | | | | | DVLDQRAHALELQRERLTLADA |
| | | | | | LAQAQAELQQLPVSLRQQLALA |
| | | | | | GASLQADRRTAIEQAAASRSEVR |
| | | | | | APRAGRVALRPLQRGQAVGQGQ |
| | | | | | RLADLLPTSTATEVVLYAPSRAA |
| | | | | | GLIGPGIPVQLRFDALPYQHYGQ |
| | | | | | FAGRVVEIAAAPEPPRADAALAS |
| | | | | | EPLYRVRVRLAGDAALRAGHAA |
| | | | | | VLRPGMRVQGTLALEWRRFSQ |
| | | | | | WAFEPLSSLHGTLR |
| 2 | WP_033013179.1 | HlyD family | Xanthomonas | 234 | MDAQQRSRLGPVVLRQRGALG |
| | | efflux | oryzae | | WCVGVLMATVILLLVGFFCLGF |
| | | transporter | | | ARRQTLYGAVVPADGMIAITTPQ |
| | | periplasmic | | | SGVVANVGVVQGQRVAAGQVL |
| | | adaptor | | | FVLAAEHRDDRGRPSQQAAAVL |
| | | subunit | | | AEQQRLTAEAMVQLRAQGRLQ |
| | | | | | QQAAARALAGLRNRLEQVDAEL |
| | | | | | GVLRHRQQLTQSIEQRYRTALTR |
| | | | | | GLVSQQFVDEKQADVLDQRAH |
| | | | | | ALELQRERLTLADALAQAQAEL |
| | | | | | QQLPVSLRQQLALAGASLQADR |
| | | | | | RTAIEQAAASRWEVRAPRAGRV |
| | | | | | ALRPLQRGQAVGQGQRLADLLP |
| | | | | | TSTATEVVLYAPSRAAGLIGPGIP |
| | | | | | VQLRFDALPYQHYGQFAGRVVE |
| | | | | | IAAVPEPPRADAPLASEPLYRMR |
| | | | | | VRLAGDAALRAGHAAVLRPGM |
| | | | | | RVQGTLALEWRRFSQWAFEPLS |
| | | | | | SLHGTLR |
| 2 | WP_041182067.1 | HlyD family | Xanthomonas | 235 | MTLFREEVMQARRNSWLGGISL |
| | | efflux | oryzae | | AQPVRAWVLASAAGIVAASVVV |
| | | transporter | | | FLCMGTYAHRSAVTGQLVPIKG |
| | | periplasmic | | | LATVMAPATGVVSRLDVSEGQP |
| | | adaptor | | | VKAGQMLAVVAVPRATLDSGD |
| | | subunit | | | TQAALQHQLRKRGEGVKSSEQA |
| | | | | | QLQQLDAQEQGLRAQLANLRQE |
| | | | | | LMQVDAEIATRRNQIALANEVL |
| | | | | | QRWRQLQDDKYVSVLQIKQQES |
| | | | | | TALEYTSQMQALQRQATEMRRS |
| | | | | | AAQIEQQLRVLPGQRGGVQADY |
| | | | | | RRDAAAVEQEQVQTQVNGALV |
| | | | | | VTAPVAGVVATQMAKPGQAIQL |
| | | | | | GQPLLSVVPGDGRLEAELLVPSR |
| | | | | | AIGFIAPGDTVLLRYQAFPYQKF |
| | | | | | GHQQGRLSRISRSALSSSELGALI |
| | | | | | GNVQQGEPYYRVTVGLGRQSV |
| | | | | | MAYGKPEMLKPGMLLQADIIGE |
| | | | | | RRKLIEWVFEPLLSLGR |
| 2 | WP_044756939.1 | HlyD family | Xanthomonas | 236 | MTLFRQEVLQARRNSWLGGISL |
| | | efflux | oryzae | | AQPVRAWVLASAAGIVAGSVVV |
| | | transporter | | | FLCMGTYAHRSTVTGQLVPIKGL |
| | | periplasmic | | | ATVMAPATGVVSRLDVSEGQPV |
| | | adaptor | | | KAGQMLAVVAVPRATLDSGDT |
| | | subunit | | | QAALQHQLRKRGEGLKSSEQAQ |
| | | | | | LQQLDAQEQGLRAQLANLRQEL |
| | | | | | MQVDAEIATRRNQIALANEVLQ |
| | | | | | RWRQLQDDKYVSVLQIKQQESS |
| | | | | | ALEYTSQMQALQRQATEMRRSA |
| | | | | | AQIEQQLRVLPGQRGGVQADYR |
| | | | | | RDAAAVEQEQVQTQVNGALVV |
| | | | | | TAPVAGVVATQMAKPGQAIQLG |
| | | | | | QPLLSVVPGDGHLEAELLVPSRA |
| | | | | | IGFIAPGDTVLLRYQAFPYQKFG |
| | | | | | HQQGLLSRISRSALSSSELGALIG |
| | | | | | NAQQGEPYYRVTVALARQSVM |
| | | | | | AYGKPEMLKPGMLLQADIIGER |
| | | | | | RKLIEWVFEPLLSLGR |
| 2 | WP_053502543.1 | HlyD family | Xanthomonas | 237 | MTLFRQEVLQARRNSWLGGISL |
| | | efflux | oryzae | | AQPVRAWVLASAAVIVAAFVVV |
| | | transporter | | | FLCMGTYAHRSTVTGQLVPIKGL |
| | | periplasmic | | | ATVMAPATGVVSRLKVSEGQHV |
| | | adaptor | | | KAGQMLGVVTVPRATLGSGDTQ |
| | | subunit | | | AALQHQLRQRGEGVKSSEQAQL |
| | | | | | QQLDAQEQGLRAQLANLCQELV |
| | | | | | QVDAEVATRRNQIALANEVLQR |
| | | | | | WRQLQDDKYVSVLQIKQQESSA |
| | | | | | LEYTSQMQALQRQATEMRRSAA |
| | | | | | QIEQQLRVLPGQRGSVQADYRR |
| | | | | | DAAAVEQEQVQTQVNGALVVT |
| | | | | | APVAGVVATQMAKPGQAIQLGQ |
| | | | | | PLLSVVPGDGRLEAELLVPSRAI |
| | | | | | GFIAPGDTVLLRYQAFPYQKFGH |
| | | | | | QQGRLSRISRSALSSSELGALIGN |
| | | | | | AQQGEPYYRVTVALGRQSVMA |
| | | | | | YGKPEMLKPGMLLQADIIGERR |
| | | | | | KLIEWVFEPLLSLGK |
| 2 | WP_075239140.1 | HlyD family | Xanthomonas | 238 | MTLFREEVMQARRNSWLGGISL |
| | | efflux | oryzae | | AQPVRAWVLASAAGIVAASVVV |
| | | transporter | | | FLCMGTYAHRSTVTGQLVPIKGL |
| | | periplasmic | | | ATVMAPATGVVSRLDVSEGQPV |
| | | adaptor | | | KAGQMLAVVAVPRATLDSGDT |
| | | subunit | | | QAALQHQLRKRGEGLKSSEQAQ |
| | | | | | LQQLDAQEQGLRAQLANLRQEL |
| | | | | | MQVDAEIATRRNQIALANEVLQ |
| | | | | | RWRQLQDDKYVSVLQIKQQESS |
| | | | | | ALEYTSQMQVLQRQATEMRRSA |
| | | | | | AQIEQQLRVLPGQRGGVQADYR |
| | | | | | RDAAAVEQEQVQTQVNGALVV |
| | | | | | TAPVAGVVATQMAKPGQAIQLG |
| | | | | | QPLLSVVPGDGRLEAELLVPSRA |
| | | | | | IGFIAPGDTVLLRYQAFPYQKFG |
| | | | | | HQQGLLSRISRSALSSSELGALIG |
| | | | | | NAQQGEPYYRVTVALARQSVM |
| | | | | | AYGKPEMLKPGMLLQADIIGER |
| | | | | | RKLIEWVFEPLLSLGR |
| 2 | WP_149621440.1 | HlyD family | Xanthomonas | 239 | MTLFREEVMQARRNSWLGVISL |
| | | efflux | oryzae | | AQPVRAWVLASAAGIVAASVVV |
| | | transporter | | | FLCMGTYAHRSTVIGQLVPIKGL |
| | | periplasmic | | | ATVMAPATGVVSRLDVSEGQPV |
| | | adaptor | | | KAGQMLAVVAVPRATLDSGDT |
| | | subunit | | | QAALQHQLRKRGEGLKSSEQAQ |
| | | | | | LQQLDAQEQGLRAQLANLRQEL |
| | | | | | MQVDAEIATRRNQIALANEVLQ |
| | | | | | RWRQLQDDKYVSVLQIKQQESS |
| | | | | | ALEYTSQMQALQRQATEMRRSA |
| | | | | | AQIEQQLRVLPGQRGGVQADYR |
| | | | | | RDAAAVEQEQVQTQVNGALVV |
| | | | | | TAPVAGVVATQMAKPGQAIQLG |
| | | | | | QPLLSVVPGDGHLEAELLVPSRA |
| | | | | | IGFIAPGDTVLLRYQAFPYQKFG |
| | | | | | HQQGLLSRISRSALSSSELGALIG |
| | | | | | NAQQGEPYYRVTVALARQSVM |
| | | | | | AYGKPEMLKPGMLLQADIIGER |
| | | | | | RKLIEWVFEPLLSLGR |
| 2 | WP_178385557.1 | HlyD family | Xanthomonas | 240 | MTRLFRREVMDAQQRSRLGPVV |
| | | efflux | oryzae | | LRQRGALGWCVGVLMATVILLL |
| | | transporter | | | VGFFCLGFARRQTLYGAVVPAD |
| | | periplasmic | | | GMIAITTPQSGVVANVGVVQGQ |
| | | adaptor | | | RVAAGQVLFVLAAEHRDDRGRP |
| | | subunit | | | SQQAAAVLAEQQRLTAEAMVQ |
| | | | | | LRAQGRLQQQAAARALAGLRN |
| | | | | | RLEQVDAELGVLRHRQQLTQSIE |
| | | | | | QRYRTALTRGLVSQQFVDEKQA |
| | | | | | DVLDQRAHALELQRERLTLADA |
| | | | | | LAQAQAELQQLPVSLRQQLALA |
| | | | | | GASLQADRRTAIEQAAASRWEV |
| | | | | | RAPRAGRVALRPLQRGQAVGQG |
| | | | | | QRLADLLPTSTATEVVLYVPSRA |
| | | | | | AGLIGPGIPVQLRFDALPYQHYG |
| | | | | | QFAGRVVEIAAVPEPPRADAALA |
| | | | | | SEPLYRMRVRLAGDAALRAGHA |
| | | | | | AVLRPGMRVQGTLALEWRRFSQ |
| | | | | | WAFEPLSSLHGTLR |
| 2 | WP_207924419.1 | HlyD family | Xanthomonas | 241 | MTRLFRREVMDAQQRSRLGPVV |
| | | efflux | oryzae | | LRQRGALGWCVGVLMATVILLL |
| | | transporter | | | VGFFCLGFARRQTLYGTVVPAD |
| | | periplasmic | | | GMIAITTPQSGVVANVGVVQGQ |
| | | adaptor | | | RVAAGQVLFVLAAEHRDDRGRP |
| | | subunit | | | SQQAAAVLAEQQRLTAEAMVQ |
| | | | | | LRAQGRLQQQAAARALAGLRN |
| | | | | | RLEQVDAELGVLRHRQQLTQSIE |
| | | | | | QRYRTALTRGLVSQQFVDEKQA |
| | | | | | DVLDQRAHALELQRERLTLADA |
| | | | | | LAQAQAELQQLPVSLRQQLALA |
| | | | | | GASLQADRRTAIEQAAASRWEV |
| | | | | | RAPRAGRVALRPLQRGQAVGQG |
| | | | | | QRLADLLPTSTATEVVLYAPSRA |
| | | | | | AGLIGPGIPVQLRFDALPYQHYG |
| | | | | | QFAGRVVEIAAAPEPPRADAALA |
| | | | | | SEPLYRVRVRLAGDAALRAGHA |
| | | | | | AVLRPGMRVQGTLALEWRRFSQ |
| | | | | | WAFEPLSSLHGTLR |
| 2 | WP_017114665.1 | HlyD family | Xanthomonas | 242 | MSLFRQEVLQARRNSWLGGISL |
| | | efflux | vasicola | | AQPVRAWMLASAAVIVAASVV |
| | | transporter | | | VFLCMGTYAHRSTVTGQLVPIK |
| | | periplasmic | | | GLATVMAPATGVVSRLEVSEGQ |
| | | adaptor | | | PVKAGQILGVVTVPRATLGSGDT |
| | | subunit | | | QAALQHQLRQRGEGLKSSEQAQ |
| | | | | | LQQLDAQEQGLHAQLANLRQEL |
| | | | | | VQVDAEIATRRNQIALANEVLQR |
| | | | | | WRQLQDDKYVSVLQIKQQESSA |
| | | | | | LEYTSQMQALQRQATEMRRSAA |
| | | | | | QIEQQLRVLPGQRGGVQADYRR |
| | | | | | DAAAVEQEQVQTQVNGALVVT |
| | | | | | APVAGVVATQMAKPGQAIQLGQ |
| | | | | | PLLSVVPGDGRLEAELLVPSRAI |
| | | | | | GFIAPGDTVLLRYQAFPYQKFGH |
| | | | | | QQGRLSRISRSALSSSELGALIGN |
| | | | | | AQQGEPYYRVTVALGRQSVMA |
| | | | | | YGKPEMLKPGMLLQADIIGERR |
| | | | | | KLIEWVFEPLLSLGR |
| 2 | WP_026113264.1 | HlyD family | Xanthomonas | 243 | MEPCWYEQTRGLATHGGAPTAL |
| | | efflux | vasicola | | PSRRAARPGVGGCGRMTTLFRR |
| | | transporter | | | EAMDYRQRSRLGPVVLGQHRAL |
| | | periplasmic | | | RWCVGLLMAAVILLLIGFFRLGF |
| | | adaptor | | | ARSKTLHGTVVPADGMIALTTP |
| | | subunit | | | QSGVVVQVGAVQGQVVAAGQ |
| | | | | | MLFVLEAEHRDDRGRPTRRAAA |
| | | | | | VLAEQQRLAVEAMAQLRAQGR |
| | | | | | MQQQAAARALAGLRDRLQQVD |
| | | | | | AELDLLRHRQQLTQSIEQRYRTA |
| | | | | | LTRGLVSQQFVDEKQADVLDQR |
| | | | | | THTLEVERERMALADALAQAQV |
| | | | | | ELQQLPSSLRQQLAIAGAGLQED |
| | | | | | RRAAIEQAAASRWEVRAPRAGR |
| | | | | | MALRPLQRGQAVVQGQRLADL |
| | | | | | LPTSIATEVVLYAPSGAAGLIGPG |
| | | | | | MPVQLRFDALPYQHYGQFAGRV |
| | | | | | VEIAAAPEPARVDSASASEPLYR |
| | | | | | VRVRLAGDAALRTGRMAVLRP |
| | | | | | GMRVQGTLALEWRRFSQWAFEP |
| | | | | | LSNVYGTLR |
| 2 | WP_039431015.1 | HlyD family | Xanthomonas | 244 | MTLFRQEVLQARHNSWLGGISL |
| | | efflux | vasicola | | AQPVRAWMLASAAVIVAASVV |
| | | transporter | | | VFLCLGTYAHRSTVTGHLVPIKG |
| | | periplasmic | | | LSTVMAPATGVVSRLEVSEGQP |
| | | adaptor | | | VKAGQILGVVTVPRATLGSGDT |
| | | subunit | | | QAALQHQLRQRGEGLKSSEQAQ |
| | | | | | LQQLDAQEQGLRAQLANLRQEL |
| | | | | | VQVDAEIATRRNQIALANEVLQR |
| | | | | | WRQLQDDKYVSVLQIKQQESSA |
| | | | | | LEYTSQMQALQRQATEMRRSAA |
| | | | | | QIEQQLRVLPGQRGGVQADYRR |
| | | | | | DAAAVEQEQVQTQVNGALVVT |
| | | | | | APVAGVVATQMAKPGQAIQLGQ |
| | | | | | PLLSVVPGDGRLEAELLVPSRAI |
| | | | | | GFIAPGDTVLLRYQAFPYQKFGH |
| | | | | | QQGRLSRISRSALSSSELGALIGN |
| | | | | | VQQGEPYYRVTVALGRQSVMA |
| | | | | | YGKPEMLKPGMLLQADIIGERRR |
| | | | | | LIEWVFEPLFSLDK |
| 2 | WP_172645741.1 | HlyD family | Xanthomonas | 245 | MEPCWYEQTRGLATHGGAPTAL |
| | | efflux | vasicola | | PSRRAARPGVGGCGRMTTLFRR |
| | | transporter | | | EAMDYRQRSRLGPVVLRQHRAL |
| | | periplasmic | | | RWCVGLLMAAVILLLIGFFRLGF |
| | | adaptor | | | ARSKTLHGTVVPADGMIALTTP |
| | | subunit | | | QSGVVVQVGAVQGQVVAAGQ |
| | | | | | MLFVLEAEHRDDRGRPTRRAAA |
| | | | | | VLAEQQRLAVEAMAQLRAQGR |
| | | | | | MQQQAAARALAGLRDRLQQVD |
| | | | | | AELDLLRHRQQLTQSIEQRYRTA |
| | | | | | LTRGLVSQQFVDEKQADVLDQR |
| | | | | | THTLEVERERMALADALAQAQA |
| | | | | | ELQQLPSSLRQQLAIAGAGLQED |
| | | | | | RRAAIEQAAASRWEVRAPRAGR |
| | | | | | MALRPLQRGQAVVQGQRLADL |
| | | | | | LPTSIATEVVLYAPSGAAGLIGPG |
| | | | | | MPVQLRFDALPYQHYGQFAGRV |
| | | | | | VEIAAAPEPARVDSASASEPLYR |
| | | | | | VRVRLAGDAALRAGRMAVLRP |
| | | | | | GMRVQGTLALEWRRFSQWAFEP |
| | | | | | LSNVYGTLR |
| 2 | WP_193588616.1 | HlyD family | Xanthomonas | 246 | MEPCWYEQTRGLATHGGAPTAL |
| | | efflux | vasicola | | PSRRAARPGVGSCGRMTTLFRRE |
| | | transporter | | | AMDYRQRSRLGPVVLRQHRALR |
| | | periplasmic | | | WCVGLLMAAVILLLIGFFRLGFA |
| | | adaptor | | | RSKTLHGTVVPADGMIALTTPQS |
| | | subunit | | | GVVVQVGAVQGQVVAAGQMLF |
| | | | | | VLEAEHRDDRGRPTRRAAAVLA |
| | | | | | EQQRLAVEAMAQLRAQGRMQQ |
| | | | | | QAAARALAGLRDRLQQVDAEL |
| | | | | | DLLRHRQQLTQSIEQRYRTALTR |
| | | | | | GLVSQQFVDEKQADVLDQRTHT |
| | | | | | LEVERERMALADALAQAQAELQ |
| | | | | | QLPSSLRQQLAIAGAGLQEDRRA |
| | | | | | AIEQAAASRWEVRAPRAGRMAL |
| | | | | | RPLQRGQAVAQGQRLADLLPTSI |
| | | | | | ATEVVLYAPSGAAGLIGPGMPV |
| | | | | | QLRFDALPYQHYGQFAGRVVEI |
| | | | | | AEAPEPARVDSASASEPLYRVRV |
| | | | | | RLAGDAALRTGRMAVLRPGMR |
| | | | | | VQGTLALEWRRFSQWAFEPLSN |
| | | | | | VYGTLR |
| 2 | WP_004086573.1 | HlyD family | Xylella | 247 | MKDLFRKEVLEAKRTSWLGGIS |
| | | efflux | fastidiosa | | LIQPIKAWLLVFASSVMALLVVV |
| | | transporter | | | FLCVGTYTRRSTVTGQLVPSKGL |
| | | periplasmic | | | VTVVAPATGMVSHIKVSEGANA |
| | | adaptor | | | LSGQILALVTVPRTTLASGDAIR |
| | | subunit | | | ALEQQLQERKQGLSMGQQAQA |
| | | | | | QVLSAQTSGIEAQLSAAQRELAQ |
| | | | | | IEIEIATRQKQIQIANETLQRLKQL |
| | | | | | EDARYVGTLQIKQQESTVLDYTS |
| | | | | | QMQAMQRQAMMTRRSIAQLQQ |
| | | | | | SLRELPGQYQNSQATYRRELAQ |
| | | | | | LEQEKVETEARGALSVVSPIDGI |
| | | | | | VATQIVKSQQTVQAGQPMLSLL |
| | | | | | PVNGELEAELLLPSSAIGFIEQGD |
| | | | | | EVRLRYQAYPYQKFGHQRGQVS |
| | | | | | RISRSALTSSELAALIGNTQQTEP |
| | | | | | YYRVVVKLPQQSITVYGRHEPL |
| | | | | | KPGMLIDADILSEKRRLIEWIFEP |
| | | | | | LYSLRGKVSDN |
| 2 | WP_010893724.1 | HlyD family | Xylella | 248 | MKDLFRKEVLEAKRTSWLGGIS |
| | | efflux | fastidiosa | | LIQPIKAWLLVLASSVMALLVVV |
| | | transporter | | | FLYVGTYTRRSSVTGQLVPSKGL |
| | | periplasmic | | | VTVVAPATGMVSQIKVSEGANA |
| | | adaptor | | | LSGQILALVTVPRTTLASGDATR |
| | | subunit | | | ALEQQLQERKQGLSMGQQAQA |
| | | | | | QVLSAQTSGIEAQLSAAQRELAQ |
| | | | | | IEIEIATRQKQIQIANETLQRLKQL |
| | | | | | EDARYVGTLQIKQQESTVLDYTS |
| | | | | | QMQAMQRQAIMTRRSIAQLQQS |
| | | | | | LRELPGQYQNSQATYRRELAQL |
| | | | | | EQEKVETEARGALSVVSPIDGIV |
| | | | | | ATQIVKSQQTVQAGQPMLSLLP |
| | | | | | VNGELEAELLLPSSAIGFIEQGDE |
| | | | | | VRLRYQAYPYQKFGHQRGQVSR |
| | | | | | ITRSALTSNELGALIGNTQQTQPY |
| | | | | | YRVIVKLPQQSITVYGRHEPLKP |
| | | | | | GMLIDADILSEKRRLIEWIFEPLY |
| | | | | | SLRGKVSDN |
| 2 | WP_011097661.1 | HlyD family | Xylella | 249 | MKDLFRKEVLEAKRTSWLGGIS |
| | | efflux | fastidiosa | | LIQPIKAWLLVFASSVMALLVVV |
| | | transporter | | | FLCVGTYTRRSSVTGQLVPSKGL |
| | | periplasmic | | | VTVVAPATGMVSQIKVSEGANA |
| | | adaptor | | | LSGQILALVTVPRTTLASGDAIR |
| | | subunit | | | ALEQQLQERKQGLSMGQQAQA |
| | | | | | QVLSAQTSGIEAQLSAAQRELAQ |
| | | | | | IEIEIATRQKQIQIANETLQRLKQL |
| | | | | | EDARYVGTLQIKQQESTVLDYTS |
| | | | | | QMQAMQRQAIMTRRSIAQLQQS |
| | | | | | LRELPGQYQNSQATYRRELAQL |
| | | | | | EQEKVETEARGALSVVSPIDGIV |
| | | | | | ATQIVKSQQTVQAGQPMLSLLP |
| | | | | | VNGELEAELLLPSSAIGFIEQGDE |
| | | | | | VRLRYQAYPYQKFGHQRGQVSR |
| | | | | | ISRSALTSSELAALIGNTQQTEPY |
| | | | | | YRVIVKLPQQSITVYGRHEPLKP |
| | | | | | GMLIDADILSEKRRLIEWIFEPLY |
| | | | | | SLRGKVSDN |
| 3 | H47_MFP_mchE | Microcin H47 | Escherichia | 250 | MFRQDALENRKMKWQGRAILLP |
| | | secretion | coli | | GIPLWLIMLGSIVFITAFLMFIIVG |
| | | protein MchE | | | TYSRRVNVSGEVTTWPRAVNIY |
| | | | | | SGVQGFVVRQFVHEGQLIKKGD |
| | | | | | PVYLIDISKSTRSGIVTDNHRRDI |
| | | | | | ENQLVRVDNIISRLEESKKITLDT |
| | | | | | LEKQRLQYTDAFRRSSDIIQRAE |
| | | | | | EGIKIMKNNMENYRNYQAKGLI |
| | | | | | NKDQLTNQVALYYQQQNNLLSL |
| | | | | | SGQNEQNALQITTLESQIQTQAA |
| | | | | | DFDNRIYQMELQRYELQKELVN |
| | | | | | TDVEGEIIIRALTDGKVDSLSVTV |
| | | | | | GQMVNTGDSLLQVIPENIENYYL |
| | | | | | ILWVPNDAVPYISAGDKVNIRYE |
| | | | | | AFPAEKFGQFSATVKTISRTPAST |
| | | | | | QEMLTYKGAPQNTPGASVPWY |
| | | | | | KVIAMPEKQIIRYDEKYLPLENG |
| | | | | | MKAESTLFLEKRRIYQWMLSPF |
| | | | | | YDMKHSATGPLND |
| 3 | WP_009309915.1 | MULTISPECIES: | Entero- | 251 | MFRQEALQHRNSVWKAKAILLP |
| | | HlyD | bacterales | | GIPLWCIVLSCFVFITFFIVFIISGS |
| | | family | | | YTRRINVSAEVTTWPRPIVISTSQ |
| | | secretion | | | QGYVVRSYVKEGQKIRKGDRLY |
| | | protein | | | ELDTSKTTRSGVVSDNEFNEITS |
| | | | | | QLHGIDEIIRNLEKGRDETLQSIK |
| | | | | | DQRNKYKEAYDISSSIVRTAEQG |
| | | | | | LQKMENNMRNYEQYRKNGLIN |
| | | | | | NDQLTNQMSLYYQQENSVLSIK |
| | | | | | NQNIQNALQIKNLEREVQIQTSE |
| | | | | | YDSRIYQMKLQRNELKKELIKSG |
| | | | | | LNSSIIIRSPSDGIIDTLNVSQGQIV |
| | | | | | NAGDTLSQIIPERDRKLYLILWIP |
| | | | | | DMAVPYIKKGDHVRIRYDAFAY |
| | | | | | EKFGQFNGIIHSISKSPSSQQEML |
| | | | | | SYHLLDNIKLFSGHAYYRVMVT |
| | | | | | PENDRIYYNKEEIHLENGMRAEL |
| | | | | | TLFLENRKIYQWMISPLYDVVKS |
| | | | | | AQGNIHEK |
| 3 | WP_033644082.1 | MULTISPECIES: | Entero- | 252 | MFREEALSHSRSKWAGKALLIA |
| | | HlyD | bacterales | | GLPAWVIAGISLSFVSVLIIFVTY |
| | | family | | | GGYTRRISVGGEVTTLPRSVNIF |
| | | secretion | | | ATQQGFIAQRFVNVGDQVKKGQ |
| | | protein | | | HLYQIDVSRVTDSGTVSASTRQA |
| | | | | | IENQLAQVDSIIAKLEANKKTTL |
| | | | | | VNLQAQIDQYKLAHSQSKQMVE |
| | | | | | NSRNGVNFMRETMKNYDEYRS |
| | | | | | KGLINRDQLNNQVYLYYQQQST |
| | | | | | FHSLYNQSIQESLQITNLNSDLAT |
| | | | | | RASDFDNQISQYQFQRNDLLRQL |
| | | | | | AEADASGSLIVNAPADGRVESLS |
| | | | | | VTPGQMVNTGDSLAQLIPSNHAI |
| | | | | | YYLVLWLPNSSVPYVSRGDHIN |
| | | | | | VRYDAFPYEKFGQFPGVIESIAY |
| | | | | | VPASIQEMSTYSSSPTRQSAELVE |
| | | | | | SYYKVLVAIDNTQFGYQGKKLN |
| | | | | | LSSGMKAQATLFLEKRPIYQWM |
| | | | | | FAPFYDMKNSVSGPLRE |
| 3 | WP_000489608.1 | MULTISPECIES: | Entero- | 253 | MFRQDALENRKMKWQGRAILLP |
| | | HlyD | bacteriaceae | | GIPLWLIMLGSIVFITAFLMFIIVG |
| | | family | | | TYSRRVNVSGEVTTWPRAVNIY |
| | | secretion | | | SGVQGFVVRQFVHEGQLIKKGD |
| | | protein | | | PVYLIDISKSTRNGIVTDNHRRDI |
| | | | | | ENQLVRVDNIISRLEESKKITLDT |
| | | | | | LEKQRLQYTDAFRRSSDIIQRAE |
| | | | | | EGIKIMKNNMENYRYYQSKGLI |
| | | | | | NKDQLTNQVALYYQQQNNLLSL |
| | | | | | SGQNEQNALQITTLESQIQTQAA |
| | | | | | DFDNRIYQMELQRLELQKELVN |
| | | | | | TDVEGEIIIRALSDGKVDSLSVTV |
| | | | | | GQMVNTGDSLLQVIPENIENYYL |
| | | | | | ILWVPNDAVPYISAGDKVNIRYE |
| | | | | | AFPSEKFGQFSATVKTISRTPAST |
| | | | | | QEMLTYKGAPQNTPGASVPWY |
| | | | | | KVIATPEKQIIRYDEKYLPLENG |
| | | | | | MKAESTLFLEKRRIYQWMLSPF |
| | | | | | YDMKHSATGPIND |
| 3 | WP_000489609.1 | MULTISPECIES: | Entero- | 254 | MFRQDALENRKMKWQGRAILLP |
| | | HlyD | bacteriaceae | | GIPLWLIMLGSIVFITAFLMFIIVG |
| | | family | | | TYSRRVNVSGEVTTWPRAVNIY |
| | | secretion | | | SGVQGFVVRQFVHEGQLIKKGD |
| | | protein | | | PVYLIDISKSTRNGIVTDNHRRDI |
| | | | | | ENQLVRVNNIISRLEESKKITLDT |
| | | | | | LEKQRLQYTDAFRRSSDIIQRAE |
| | | | | | EGIKIMKNNMENYRYYQSKGLI |
| | | | | | NKDQLTNQVALYYQQQNNLLSL |
| | | | | | SGQNEQNALQITTLESQIQTQAA |
| | | | | | DFDNRIYQMELQRLELQKELVN |
| | | | | | TDVEGEIIIRALSDGKVDSLSVTV |
| | | | | | GQMVNTGDSLLQVIPENIENYYL |
| | | | | | ILWVPNDAVPYISAGDKVNIRYE |
| | | | | | AFPSEKFGQFSATVKTISRTPAST |
| | | | | | QEMLTYKGAPQNTPGASVPWY |
| | | | | | KVIATPEKQIIRYDEKYLPLENG |
| | | | | | MKAESTLFLEKRRIYQWMLSPF |
| | | | | | YDMKHSATGPIND |
| 3 | WP_019725220.1 | MULTISPECIES: | Entero- | 255 | MARKLYRKEAIEYKKLHWKGK |
| | | HlyD | bacteriaceae | | ALLLAGMPAWLIVILSSCFLIALI |
| | | family | | | STLILCTFTQRIDVRGEVITLPHS |
| | | secretion | | | VNVFAPQQGFVLNQYVKVGDIV |
| | | protein | | | KKGQKLYEIDISRNTTNGNVSLA |
| | | | | | QTAVINEKIINAESIITKLIRNKDE |
| | | | | | TLNALDTQLNTIKKSLSETTSML |
| | | | | | ANTQAGLNKMHQNLSSYDKYL |
| | | | | | KEGLITKDQYNYQHSLYFQQQS |
| | | | | | AYQSLVSQKMQLESQITQFTSDK |
| | | | | | VTKAADFDNQISNQQNQINDYK |
| | | | | | NQLVESDAKGNVIIKATTDGRIE |
| | | | | | SLAVTKGQMVDNGSSLAQIKPT |
| | | | | | DNVEYYLILWLPNNSIPYVKPGD |
| | | | | | TINIRYDAFPADKFGQFPGEVISIS |
| | | | | | SMPASRQEMSEYTNVNNGTNQQ |
| | | | | | ELALYKTIVKIKQKSFSYNGKTL |
| | | | | | YLSNGLKAEAVVFLEERPLYMW |
| | | | | | MFTPFYKISQSVSGPINE |
| 3 | WP_000489611.1 | HlyD family | Escherichia | 256 | MFRQDALENRKMKWQGRAILLP |
| | | secretion | coli | | GIPLWLIMLGSIVFITAFLMFIIVG |
| | | protein | | | TYSRRVNVSGEVTTWPRAVNIY |
| | | | | | SGVQGFVVRQFVHEGQLIKKGD |
| | | | | | PVYLIDISKSTRSGIVTDNHRRDI |
| | | | | | ENQLVRVDNIISRLEESKKITLDT |
| | | | | | LEKQRLQYTDAFRRSSDIIQRAE |
| | | | | | EGIKIMKNNMENYRNYQAKGLI |
| | | | | | NKDQLTNQVALYYQQQNNLLSL |
| | | | | | SGQNEQNALQITTLESQIQTQAA |
| | | | | | DFDNRIYQMELQRYELQKELVN |
| | | | | | TDVEGEIIIRALTDGKVDSLSVTV |
| | | | | | GQMVNTGDSLLQVIPENIENYYL |
| | | | | | ILWVPNDAVPYISAGDKVNIRYE |
| | | | | | AFPAEKFGQFSATVKTISKTPAST |
| | | | | | QEMLTYKGAPQNTPGDSVPWY |
| | | | | | KVIAMPEKQIIRYDEKYLPLENG |
| | | | | | MKAESTLFLEKRRIYQWMLSPF |
| | | | | | YDMKHSATGPLND |
| 3 | WP_004177748.1 | MULTISPECIES: | Klebsiella | 257 | MLKKIYRKEAIEYKKHHWKGKA |
| | | HlyD | | | LLLAGLPAWLVALLSFAFLAILIA |
| | | family | | | TTVFCSFTQRIDVQGEVITLPHSV |
| | | secretion | | | NVYAPQQGFVISQYVKVGDIVT |
| | | protein | | | KGQPLYEIDISRNTTTGNVSAVQI |
| | | | | | EVINEKIANAEDIISKLNHNKEET |
| | | | | | TISLEKQLKTINDSLKETNRMLA |
| | | | | | NAQAGLKKMHDNLSSYDKYLS |
| | | | | | DGLITKDQYNYQHSLYFQQQST |
| | | | | | YQSLVSQKMQLESQVTQLNSDK |
| | | | | | ITKIADFDNQISSQENQINDYKNQ |
| | | | | | LVESNANGNIIIKATTEGRIESLT |
| | | | | | VTKGQMVDKGSSLAQIKPTGDIE |
| | | | | | YYLILWLPNNSIPYVKPGDEINIR |
| | | | | | YAAFPSDKFGQFPGKILSISSVPT |
| | | | | | SRQEMSEYTNVINGTNQQELAL |
| | | | | | YKTIVKIENKTFEYNGKTLSLSN |
| | | | | | GLKAQAVVFLEERPLYMWMFTP |
| | | | | | VYKITQSITGPIHD |
| 3 | WP_004178320.1 | MULTISPECIES: | Klebsiella | 258 | MTRRLYRKEAIEYKRVYWKGK |
| | | HlyD | | | ALLLAGMPTWLIIVLSLLFLGLLI |
| | | family | | | SAMIFCSFTQRIDVNGEVITLPHS |
| | | secretion | | | VNVYAPQQGFIVSQYVKVGDLV |
| | | protein | | | KKGQPLYEIDISRDTTTGNVSSV |
| | | | | | QIEVNKEKIANAEEIINKLSQNKQ |
| | | | | | ETLSSIEKQLKANKESLKEINRLL |
| | | | | | ANALAGLKKMHDNLSSYDSYLR |
| | | | | | DGLITKDQYNYQHSLYFQQQST |
| | | | | | YQSLVSQKMQLESQITQYNSDKI |
| | | | | | TKIADFDNQITAQQNQINDFKNQ |
| | | | | | LVESNAKGNAIIKATTEGRIESLS |
| | | | | | VTKGQMVDNGSSLAQIKPTGNIE |
| | | | | | YYLILWLPNNSIPYVKPGDVINIR |
| | | | | | YDAFPSDKFGQFPGEVISISSVPA |
| | | | | | ARQEMSEYTNVSNGTNQQELAL |
| | | | | | YKTIVKIKNKTFSYNGKTLKLSN |
| | | | | | GLKARAVVFLEKRPLYMWMFTP |
| | | | | | FYKMSQSVSGPIHD |
| 3 | WP_004186616.1 | MULTISPECIES: | Klebsiella | 259 | MARKLYRKEAIEYKKLHWKGK |
| | | HlyD | | | ALLLAGMPAWLIVILSSCFLIALI |
| | | family | | | STLILCTFTQRIDVRGEVITLPHS |
| | | secretion | | | VNVFAPQQGFVLNQYVKVGDIV |
| | | protein | | | KKGQKLYEIDISRNTINGNVSLA |
| | | | | | QTAVINEKIINAESIITKLIRNKDE |
| | | | | | TLNALDTQLNTIKKSLSETTSML |
| | | | | | ANTQAGLNKMHQNLSSYDKYL |
| | | | | | KEGLITKDQYNYQHSLYFQQQS |
| | | | | | AYQSLVSQKMQLESQITQFTSDK |
| | | | | | VTKAADFDNQISNQQNQINDYK |
| | | | | | NQLVESDAKGNVIIKATTDGRIE |
| | | | | | SLAVTKGQMVDNGSSLAQIKPT |
| | | | | | GNVEYYLILWLPNNSIPYVKPGD |
| | | | | | TINIRYDAFPADKFGQFPGEVISIS |
| | | | | | SMPASRQEMSEYTNVNNGTNQQ |
| | | | | | ELALYKTIVKIKQKSFSYNGKTL |
| | | | | | YLSNGLKAEAVVFLEERPLYMW |
| | | | | | MFTPFYKISQSVSGPINE |
| 3 | WP_009309351.1 | MULTISPECIES: | Klebsiella | 260 | MARKLYRKEAIEYKKLHWKGK |
| | | HlyD | | | ALLLAGMPAWLIVTLSSCFLIALI |
| | | family | | | STLILCTFTQRIDVRGEVITLPHS |
| | | secretion | | | VNVFAPQQGFVLNQYVKVGDIV |
| | | protein | | | KKGQKLYEIDISRNTTNGNVSLA |
| | | | | | QTAVINEKIINAESIITKLIRNKDE |
| | | | | | TLNALDTQLNTIKKSLSETTSML |
| | | | | | ANTQAGLNKMHQNLSSYDKYL |
| | | | | | KEGLITKDQYNYQHSLYFQQQS |
| | | | | | AYQSLVSQKMQLESQITQFTSDK |
| | | | | | VTKAADFDNQISNQQNQINDFK |
| | | | | | NQLVESDAKGNVIIKATTDGRIE |
| | | | | | SLAVTKGQMVDNGSSLAQIKPT |
| | | | | | GNVEYYLILWLPNNSIPYVKPGD |
| | | | | | TINIRYDAFPADKFGQFPGEVISIS |
| | | | | | SMPASRQEMSEYTNVNNGTNQQ |
| | | | | | ELALYKTIVKIKQKSFSYNGKTL |
| | | | | | YLSNGLKAEAVVFLEERPLYMW |
| | | | | | MFTPFYKISQSVSGPINE |
| 3 | WP_016338369.1 | MULTISPECIES: | Klebsiella | 261 | MFRQEALQHRNSVWKAKAILLP |
| | | HlyD | | | GIPLWCIVLSCFVFITFFIVFIISGS |
| | | family | | | YTRRINVYAEVTTWPRPIVISTSQ |
| | | secretion | | | QGYVVRSYVKEGQKIRKWDRL |
| | | protein | | | YELDTSKTTRSGVVSDNEFNEIT |
| | | | | | SQLHGIDEIIRNLEKGRDETLQSI |
| | | | | | KDQRDKYKEAYDISSSIVRTAEQ |
| | | | | | GLQKMENNMRNYEQYRKSGLIN |
| | | | | | NDQLTNQMSLYYQQENSVLSIK |
| | | | | | NQNIQNALQIKNLEREVQIQTSE |
| | | | | | YDSRIYQMKLQRNELKKELIKSG |
| | | | | | LNSSIIIRSPSDGIIDTLNVSQGQIV |
| | | | | | NAGDTLSQIIPEEDRKLYLILWIP |
| | | | | | DMAIPYIKKGDRVKIRYDAFAYE |
| | | | | | KFGQFNGIIQSISKSPSSQQEMLS |
| | | | | | YHLLDNIKLFSGHAYYRVMVTP |
| | | | | | ENDRIFYNKEEIHLENGMRAEIT |
| | | | | | LFLENRKIYQWMISPLYDVVKSA |
| | | | | | QGNIHEK |
| 3 | WP_020723552.1 | MULTISPECIES: | Klebsiella | 262 | MLRKIYRKEAIEYKKHHWKGKA |
| | | HlyD | | | LLLAGIPAWLVALLSFAFLTILIA |
| | | family | | | TTILCSFTQRIDVRGEVITLPHSV |
| | | secretion | | | NVYAPQQGFVISQYAKVGDIVV |
| | | protein | | | KGQPLYEIDISRNTTSGNVSASQI |
| | | | | | EVINEKIANAEDIITKLSHNKDET |
| | | | | | RLSLDKQLQTINDSLKETNRMLV |
| | | | | | NAQTGLKKMHDNLSSYDKYLSD |
| | | | | | GLITKDQYNYQHSLYFQQQSTY |
| | | | | | QSLVSQKMQLESQITQLNSDRIT |
| | | | | | KIADFDNQISSQENQINDYKNQL |
| | | | | | VESNANGNVIIKATTEGRIESLAV |
| | | | | | TKGQMVDKGSSLAQIQPTGDIEY |
| | | | | | YLILWLPNNSIPYVKPGDIINIRY |
| | | | | | DAFPADKFGQFPGKILSISSVPTS |
| | | | | | RQELSEYTNVNNGTNQQELALY |
| | | | | | KTIVKIENKTFEYNGKTLSLSNG |
| | | | | | LKAQAVVFLEERPLYMWMFTPV |
| | | | | | YKITQSITGPIND |
| 3 | WP_023291696.1 | MULTISPECIES: | Klebsiella | 263 | MLRKIYRKEAIEYKKHHWKGKA |
| | | HlyD | | | LLLAGLPAWLIALLSFAFLAILIT |
| | | family | | | TTIFCTFTQRIDVRGEVITLPHSV |
| | | secretion | | | NVYAPQQGFVISQYAKVGDLVA |
| | | protein | | | KGQSLYEIDISRNTTTGNVSTAQI |
| | | | | | EVINEKIANAEDIITKLSHNKEET |
| | | | | | RLSLDKQLKTINDSLKETNRMLV |
| | | | | | NAQTGLKKMHDNLSSYDKYLR |
| | | | | | DGLITKDQYNYQHSLYFQQQST |
| | | | | | YQSLVSQKMQLESQVTQLNSDK |
| | | | | | ITKTADFDNQISSQENQINDYKN |
| | | | | | QLVESNANGNVIIKATTEGRIESL |
| | | | | | AVTKGQMVDKGSSLAQIKPTGD |
| | | | | | IEYYLILWLPNNSIPYVKPGDVIN |
| | | | | | IRYDAFPSDKFGQFPGKILSISSVP |
| | | | | | TSRQELSEYTNVNNGTNQQELA |
| | | | | | LYKTIVKIENKTFNYNGKTLSLS |
| | | | | | NGLKAQAVVFLEERPLYMWMF |
| | | | | | TPVYKITQSITGPIHD |
| 3 | WP_023302371.1 | MULTISPECIES: | Klebsiella | 264 | MARKLYRKEAIEYKKLHWKGK |
| | | HlyD | | | ALLLAGMPAWLIVILSSCFLIALI |
| | | family | | | STLILCTFTQRIDVRGEVITLPHS |
| | | secretion | | | VNVFAPQQGFVLNQYVKVGDIV |
| | | protein | | | KKGQKLYEIDISRNTTNGNVSLA |
| | | | | | QTAVINEKIINAESIITKLIRNKDE |
| | | | | | TLNALNTQLNTIKKSLSETTSML |
| | | | | | ANTQAGLNKMHQNLSSYDKYL |
| | | | | | KEGLITKDQYNYQHSLYFQQQS |
| | | | | | AYQSLVSQKMQLESQITQFTSDK |
| | | | | | VTKAADFDNQISNQQNQINDYK |
| | | | | | NQLVESDAKGNVIIKATTDGRIE |
| | | | | | SLAVTKGQMVDNGSSLAQIKPT |
| | | | | | GNVEYYLILWLPNNSIPYVKPGD |
| | | | | | TINIRYDAFPADKFGQFPGEVISIS |
| | | | | | SMPASRQEMSEYTNVNNGTNQQ |
| | | | | | ELALYKTIVKIKQKSFSYNGKTL |
| | | | | | YLSNGLKAEAVVFLEERPLYMW |
| | | | | | MFTPFYKISQSVSGPINE |
| 3 | WP_032426190.1 | MULTISPECIES: | Klebsiella | 265 | MLKKLYRKEAVEYKKSHWRGK |
| | | HlyD | | | ALLLNGLPAWLILLLSTLFLMALI |
| | | family | | | SALIFGKFTQRIDVKGEVITLPHSI |
| | | secretion | | | NVFSPQQGFIIKQYVKIGDIVKKD |
| | | protein | | | QALYEIDVSRNTSSGNVSAAQIA |
| | | | | | SINDKIENSNDIIKKLIDNKSQTIN |
| | | | | | ALNEQLKTSTDSLKETNRMLQN |
| | | | | | TQAGLKKMHANLASYDKYLSD |
| | | | | | GLITKDQYNYQHSLYFQQQSAY |
| | | | | | QSLVSQKMQLESQITQLNSDRVT |
| | | | | | KAADFDNQISSQYNQANDYKNQ |
| | | | | | LVESNANGNLIIKATSDGRVESL |
| | | | | | SATQGQTVENGSSLAQIKPTGNV |
| | | | | | EYYLILWLPNNAIPYLKVGDPINI |
| | | | | | RYDAFPSDKFGQFPGTILSISSMP |
| | | | | | ASRQEMSEYTNVSDGTAQQELA |
| | | | | | LYKAIVKIKDKEFKHNGKTLRLS |
| | | | | | NGLKAQAVVFLEERPLYMWMF |
| | | | | | TPVYKISQSVSGPVND |
| 3 | WP_032731752.1 | MULTISPECIES: | Klebsiella | 266 | MAQNIYRKEAIEYKKYHWKGK |
| | | HlyD | | | ALLLAGMPAWLITLLACTFLIVLI |
| | | family | | | FMLIFFNYTQRINVYGEVVTLPH |
| | | secretion | | | SINVFAPQQGFVVKQFVNIGDVV |
| | | protein | | | KKGTPLYELDVSRHTTSGNVSLA |
| | | | | | MTEVIDEKISNAKEIIQKITENKK |
| | | | | | ETVDALNDQLTSVESSLHETVR |
| | | | | | MLANTQAGLKKMRTNLSSYDD |
| | | | | | YLKKGLITKDQYNYQHNLYFQQ |
| | | | | | QSSYQSLTSQKMQLETQLTQLKS |
| | | | | | DMVTKSADFDNQISSQKNQIND |
| | | | | | YKNQRVESSANDNAIIKATADG |
| | | | | | KIESLAVTSGQMVDRGSSLAQIK |
| | | | | | PIGNVEYYLMLWLPNNSIPYIKV |
| | | | | | GDTINIRYEAFPSDKFGQFPGKV |
| | | | | | ATISTVPASRQELQEYNNVGNAA |
| | | | | | NQQDITLYKALVSIEDSSFRYND |
| | | | | | KVLTLSNGLKAEATVFLEERPLY |
| | | | | | MWMFTPFYKISHSVNGPIND |
| 3 | WP_032736708.1 | MULTISPECIES: | Klebsiella | 267 | MLKKIYRKEAIEYKKHHWKGKA |
| | | HlyD | | | LLLAGLPAWLIVLLSFSFLAIFIAT |
| | | family | | | TIFCSFTQRIDVKGEVITLPHSVN |
| | | secretion | | | VYAPQQGFVISQYVKVGDIVSK |
| | | protein | | | GQPLYEIDISRNTTTGNVSAAQIE |
| | | | | | VINEKIANAEDIIRKLSHNKEETS |
| | | | | | LSLDKQLKTINDSLKETNQMLA |
| | | | | | NAQTGLKKMHDNLASYDKYLS |
| | | | | | DGLITKDQYNYQHSLYFQQQST |
| | | | | | YQSLVSQKMQLESQVTQLNSDK |
| | | | | | ITKTADFDNQISSQENQINDYKN |
| | | | | | QLVESSANGNIIIKATTEGRIESL |
| | | | | | AVTKGQMVDKGSSLAQIKPTGD |
| | | | | | IEYYLILWLPNNSIPYVKPGDIINI |
| | | | | | RYDAFPADKFGQFPGKILSISSVP |
| | | | | | TSRQEMSEYTNVTDGTNQQELA |
| | | | | | LYKTIVKIENKSFEYNGKTLSLSN |
| | | | | | GLKAQAVVFLEERPLYMWMFTP |
| | | | | | VYKITQSITGPIND |
| 3 | WP_015368661.1 | HlyD family | Klebsiella | 268 | MNKNLYRKEAIEYKRHHWKGK |
| | | secretion | aerogenes | | ALLLAGLPAWLIALLSALFLIALI |
| | | protein | | | CALIFCKFTQRVDVKGEVITLPH |
| | | | | | SINVFAPQQGFIVKQFVKIGDVV |
| | | | | | KKNQPLYEIDVSRSTTSGNVSAA |
| | | | | | QIEVINEKIANSEDIIKKLEDNKN |
| | | | | | QTLNALNAQLKTTTASLKETNR |
| | | | | | MLVNTQVGLKKMHDNLSSYDK |
| | | | | | YLSNGLITKDQYNYQHSLYFQQ |
| | | | | | QSAYQSLVSQKMQLESQITQLNS |
| | | | | | DIITKAADFDNQISSQYNQANDY |
| | | | | | KNQLVESNANGNIIIKSTSDGRIE |
| | | | | | SLSATQGQTVDNGSSLAQIKPTG |
| | | | | | NVEYYLILWLPNNTIPYLKTGDT |
| | | | | | INIRYDAFPSDKFGQFPGKIISISS |
| | | | | | MPASRQEMSEYTNVSNGTNQQQ |
| | | | | | ELALYKAIVKIKDKQFEYDGKKL |
| | | | | | SLSNGLKAQAVVFLEERPLYMW |
| | | | | | MFTPVYKITQSVSGPIHD |
| 3 | WP_112216233.1 | HlyD family | Klebsiella | 269 | MAKGIYRKEAIEYKKYHWKGK |
| | | secretion | huaxiensis | | ALLLAGMPAWLITSVSFIFLSILIL |
| | | protein | | | TLFFCSYTQRIDVRGEVITLPHSI |
| | | | | | NVFSPQQGFIVQQYVKTGDIVKK |
| | | | | | GTPLYELDISRHTANGNVSDAIS |
| | | | | | EVITEKIINAEEIIKKIAQNKKETL |
| | | | | | DALNEQVRQFNNSLKETTKMLA |
| | | | | | NTQSGLKKMKDNLASYDLYLRQ |
| | | | | | GLITKDQYNYQHSLYFQQQSTY |
| | | | | | QSLVSQKMQQESQLTQLKSDIVT |
| | | | | | KAADFDNQISSQKNNINDFKNQ |
| | | | | | MVETNANGNVIIKATNDGKIES |
| | | | | | MAVTNGQMVDKGSSLVQIKPTG |
| | | | | | NVEYYLVLWLPNNSIPYVKKGDI |
| | | | | | INIRYDAFPSDKFGQFPGIVETISS |
| | | | | | VPASRQELSEYTNVNISNEMNQQ |
| | | | | | ELALYKTLVSIKHKSFNYNGKNL |
| | | | | | SLSNGLKAQAIVFLEKRPLYMW |
| | | | | | MFTPFYKISQSVSGPLNDQ |
| 3 | WP_004192517.1 | HlyD family | Klebsiella | 270 | MLKKIYRKEAIEYKKHHWKGKA |
| | | secretion | pneumoniae | | LLLAGLPAWLVALLSFAFLAILIA |
| | | protein | | | TTVFCSFTQRIDVQGEVITLPHSV |
| | | | | | NVYAPQQGFVISQYVKVGDIVT |
| | | | | | KGQPLYEIDISRNTTTGNVSAVQI |
| | | | | | EVINEKIANAEDIISKLNHNKEET |
| | | | | | TISLEKQLKTINDSLKETNRMLA |
| | | | | | NAQAGLKKMHDNLSSYDKYLS |
| | | | | | DGLITKDQYNYQHSLYFQQQST |
| | | | | | YQSLVSQKMQLESQVTQLNSDK |
| | | | | | ITKIADFDNQISSQENQINDYKNQ |
| | | | | | LVESNANGNIIIKATTEGRIESLT |
| | | | | | VTKGQMVDKGSSLAQIKPTGDIE |
| | | | | | YYLILWLPNNSIPYVKPGDVINIR |
| | | | | | YAAFPSDKFGQFPGKILSISSVPT |
| | | | | | SRQEMSEYTNVINGTNQQELAL |
| | | | | | YKTIVKIENKTFEYNGKTLSLSN |
| | | | | | GLKAQAVVFLEERPLYMWMFTP |
| | | | | | VYKITQSITGPIHD |
| 3 | WP_016529869.1 | HlyD family | Klebsiella | 271 | MARKLYRKEAIEYKKLHWKGK |
| | | secretion | pneumoniae | | ALLLAGMPAWLIVTLSSCFLIALI |
| | | protein | | | STLILCTFTQRIDVRGEVITLPHS |
| | | | | | VNVFAPQQGFVLNQYVKVGDIV |
| | | | | | KKGQKLYEIDISRNTTNGNVSLA |
| | | | | | QTAVINEKIINAESIITKLIRNKDE |
| | | | | | TLNALDTQLNTIKKSLSETTSML |
| | | | | | ANTQAGLNKMHQNLSSYDKYL |
| | | | | | KEGLITKDQYNYQHSLYFQQQS |
| | | | | | AYQSLVSQKMQLESQITQFTSDK |
| | | | | | VTKAADFDNQISNQQNQINDYK |
| | | | | | NQLVESDAKGNVIIKATTDGRIE |
| | | | | | SLAVTKGQMVDNGSSLAQIKPT |
| | | | | | GNVEYYLILWLPNNSIPYVKPGD |
| | | | | | TINIRYDAFPADKFGQFPGEVISIS |
| | | | | | SMPASRQEMSEYTNVNNGTNQQ |
| | | | | | ELALYKTIVKIKQKSFSYNGKTL |
| | | | | | YLSNGLKAEAVVFLEERPLYMW |
| | | | | | MFTPFYKISQSVSGPINE |
| 3 | WP_024623078.1 | HlyD family | Klebsiella | 272 | MARKLYRKEAIEYKKLHWKGK |
| | | secretion | pneumoniae | | ALLLAGMPAWLIVILSSCFLIALI |
| | | protein | | | STLILCTFTQRIDVRGEVITLPHS |
| | | | | | VNVFAPQQGFVLNQYVKVGDIV |
| | | | | | KKGQKLYEIDISRNTTNGNVSLA |
| | | | | | QTAVINEKIINAESIITKLIRNKDE |
| | | | | | TLNALNTQLNTIKKSLSETTSML |
| | | | | | ANTQAGLNKMHQNLSSYDKYL |
| | | | | | KEGLITKDQYNYQHSLYFQQQS |
| | | | | | VYQSLVSQKMQLESQITQFTSDK |
| | | | | | VTKAADFDNQISNQQNQINDYK |
| | | | | | NQLVESDAKGNVIIKATTDGRIE |
| | | | | | SLAVTKGQMVDNGSSLAQIKPT |
| | | | | | GNVEYYLILWLPNNSIPYVKPGD |
| | | | | | TINIRYDAFPADKFGQFPGEVISIS |
| | | | | | SMPASRQEMSEYTNVNNGTNQQ |
| | | | | | ELALYKTIVKIKQKSFSYNGKTL |
| | | | | | YLSNGLKAEAVVFLEERPLYMW |
| | | | | | MFTPFYKISQSVSGPINE |
| 3 | WP_046623498.1 | HlyD family | Klebsiella | 273 | MLKKIYRKEAIEYKKHHWKGKA |
| | | secretion | pneumoniae | | LLLAGLPAWLVALLSFAFLAILIA |
| | | protein | | | TTVFCSFTQRIDVQGEVITLPHSV |
| | | | | | NVYAPQQGFVISQYVKVGDIVT |
| | | | | | KGQPLYEIDISRNTTTGNVSAVQI |
| | | | | | EVINEKIANAEDIISKLNHNKEET |
| | | | | | TISLEKQLKTINDSLKETNRMLA |
| | | | | | NAQAGLKKMHDNLSSYDKYLS |
| | | | | | DGLITKDQYNYQHSLYFQQQST |
| | | | | | YQSLVSQKMQLESQVTQLNSDK |
| | | | | | ITKIADFDNQISPQENQINDYKNQ |
| | | | | | LVESNANGNIIIKATTEGRIESLT |
| | | | | | VTKGQMVDKGSSLAQIKPTGDIE |
| | | | | | YYLILWLPNNSIPYVKPGDEINIR |
| | | | | | YAAFPSDKFGQFPGKILSISSVPT |
| | | | | | SRQEMSEYTNVINGTNQQELAL |
| | | | | | YKTIVKIENKTFEYNGKTLSLSN |
| | | | | | GLKAQAVVFLEERPLYMWMFTP |
| | | | | | VYKITQSITGPIHD |
| 3 | WP_131082202.1 | HlyD family | Klebsiella | 274 | MLRKIYRKEAIEYKKHHWKGKA |
| | | secretion | pneumoniae | | LLLAGIPAWLVALLSFAFLTILIA |
| | | protein | | | TTILCSFTQRIDVRGEVITLPHSV |
| | | | | | NVYAPQQGFVISQYAKVGDIVV |
| | | | | | KGQPLYEIDISRNTTSGNVSASQI |
| | | | | | EVINEKIANAEDIITKLSHNKDET |
| | | | | | RLSLDKQLQTINDSLKETNRMLV |
| | | | | | NAQTGLKKMHDNLSSYDKYLSD |
| | | | | | GLITKDQYNYQHSLYFQQQSTY |
| | | | | | QSLVSQKMQLESQITQLNSDRIT |
| | | | | | KIADFDNQISSQENQINDYKNQL |
| | | | | | VESNANGNVIIKATTEGRIESLAV |
| | | | | | TKGQMVDKGSSLAQIQPTGDIEY |
| | | | | | YLILWLPNNSIPYVKPGDDINIRY |
| | | | | | DAFPADKFGQFPGKILSISSVPTS |
| | | | | | RQELSEYTNVNNGTNQQELALY |
| | | | | | KTIVKIENKTFEYNGKTLSLSNG |
| | | | | | LKAQAVVFLEERPLYMWMFTPV |
| | | | | | YKITQSITGPIND |
| 3 | YP_005224684.1 | microcin H47 | Klebsiella | 275 | MTNSIYRKEAIAYKKLHWKGKA |
| | | secretion | pneumoniae | | LLLAGLPAWLVTCLSLLFLCALI |
| | | protein | subsp. | | CALIFCKFTQRIDVKGEVITLPHS |
| | | | pneumoniae | | VNVFSPQQGFVVNQYVQIGDVV |
| | | | HS11286 | | KKGQTLYELDVSRNTTTGNVSA |
| | | | | | AQIEVINEKIANSEAIIKKLTHNK |
| | | | | | NETLIALDAQLKNARNSLNETVR |
| | | | | | MLANTQQGLSKMHENLSSYDK |
| | | | | | YLKEGLITKDQYNYQHSLYFQQ |
| | | | | | QSAYQSLISQKMQLETQLTQLSS |
| | | | | | DKVTKAADFDNQISSQYNQTND |
| | | | | | YKNQLVESNANGNIIIKATTEGRI |
| | | | | | ESLAVTKGQMVDKGSSLAQIKPI |
| | | | | | GNIEYYLILWLPNNSIPYVKVGD |
| | | | | | TINIRYDAFPSDKFGQFPGEIISISS |
| | | | | | LPASRQEMSEYTNVNDGTNQQE |
| | | | | | LALYKAIVKIRDKKFNYDGKELS |
| | | | | | LSNGLKAQAVVFLEERPLYMW |
| | | | | | MFTPVYKISQSVSGPVND |
| 3 | WP_087805743.1 | HlyD family | Klebsiella sp. | 276 | MARKLYRKEAIEYKKLHWKGK |
| | | secretion | PO552 | | ALLLAGMPVWLIVILSSCFLIALI |
| | | protein | | | STLILCTFTQRIDVRGEVITLPHS |
| | | | | | VNVFAPQQGFVLNQYVKVGDIV |
| | | | | | KKGLKLYEIDISRNTINGNVSLA |
| | | | | | QTAVINEKITNAESIITKLIRNKDE |
| | | | | | TLNALDTQLNTIKKSLSETTSML |
| | | | | | ANTQAGLNKMHQNLSSYDKYL |
| | | | | | KEGLITKDQYNYQHSLYFQQQS |
| | | | | | AYQSLVSQKMQLESQITQFTSDK |
| | | | | | VTKAADFDNQISNQQNQINDYK |
| | | | | | NQLVESDAKGNVIIKATTDGRIE |
| | | | | | SLAVTKGQMVDNGSSLAQIKPT |
| | | | | | GNVEYYLILWLPNNSIPYVKPGD |
| | | | | | TINIRYDAFPADKFGQFPGEVISIS |
| | | | | | SMPASRQEMSEYTNVNNGTNQQ |
| | | | | | ELALYKTIVKIKQKSFSYNGKTL |
| | | | | | YLSNGLKAEAVVFLEERPLYMW |
| | | | | | MFTPFYKISQSVSGPINE |
| 3 | WP_072046009.1 | HlyD family | Klebsiella | 277 | MYRKEAIEYKKHHWKGKALLL |
| | | secretion | variicola | | AGLPAWLIVLLSFSFLAILIATTIF |
| | | protein | | | CSFTQRIDVKGEVITLPHSVNVY |
| | | | | | APQQGFVISQYAKVGDIVNKGQ |
| | | | | | PLYEIDISRNTTTGNVSAAQIEVI |
| | | | | | NEKIANAEDIISKLSHNKEETSLS |
| | | | | | LDKQLKTINDSLKETNQMLANA |
| | | | | | QTGLKKMHDNLASYDKYLSDG |
| | | | | | LITKDQYNYQHSLYFQQQSTYQS |
| | | | | | LVSQKMQLESQVTQLNSDKITKT |
| | | | | | ADFDNQISSQENQINDYKNQLVE |
| | | | | | SSANGNIIIKATTEGRIESLAVTK |
| | | | | | GQMVDKGSSLAQIKPTGDIEYYL |
| | | | | | ILWLPNNSIPYVKPGDVINIRYDA |
| | | | | | FPADKFGQFPGKILSISSVPTSRQ |
| | | | | | EMSEYTNVTDGTNQQELALYKT |
| | | | | | IVKIENKTFEYNGKTLSLSNGLK |
| | | | | | AQAVVFLEERPLYMWMFTPVY |
| | | | | | KITQSITGPIND |
| 3 | WP_064562847.1 | HlyD family | Kosakonia | 278 | MLFRKEVAENIRSHQSGQVLLLT |
| | | efflux | oryzae | | GWP_VWLTIAVTLLFIAALLLFLIY |
| | | transporter | | | GNYTRQVNVSGEINSWPHTTDL |
| | | periplasmic | | | FAPEEGVIARVFITPGQTVRAGA |
| | | adaptor | | | ALYELDVSKTSPSGNLSSTTLAV |
| | | subunit | | | LENQSVQIDKALRNIDYNQHVTL |
| | | | | | ESLQKQLDQLSIERQKTVSMVDI |
| | | | | | SAEGVNAMRSSMKDYERYRSQ |
| | | | | | GLVTTDQQNNQRYLFYQQMSV |
| | | | | | WNSLSSQIVQQDMQINSLRSDLV |
| | | | | | TRAAQFDEQKSRYLLQQADIKR |
| | | | | | QLAEANVSKTRLITAPAAGHVSS |
| | | | | | LNITQGQMVAAGDEVVQLVPST |
| | | | | | GILFHLVLWLPDDSVPYIKPGEDI |
| | | | | | NIRYAAYPAEKYGQFPGKIISVSS |
| | | | | | APVTGSELRKRASIPQSPGKIWF |
| | | | | | KAVVAVDKEKLQWRGKRLPLV |
| | | | | | SGMQAQATLFLEKRPLYQWMF |
| | | | | | YPYYTLKNSISEPVSDKA |
| 3 | WP_064566346.1 | HlyD family | Kosakonia | 279 | MFRKEALENRKMRWRGRAILLP |
| | | secretion | oryzae | | GVPLWLIIMFCVCFLAAFLTFVID |
| | | protein | | | GTYMRRVNVSGEITTWPRPVNIY |
| | | | | | SNVQGFIVKSYVTEGQKIKKGDP |
| | | | | | VYLIDVSRSTHSGVVSDNQRNDI |
| | | | | | EQQITRVENIIAGLQESKNATLKT |
| | | | | | LEKQKQQYSEALKFSTDVIRRAE |
| | | | | | EGVKLMKENMENYRRYQARGLI |
| | | | | | TKDQLTNHVTLYYQQQNDLLGL |
| | | | | | RSQSEQNALQVTSLESEIQTQAA |
| | | | | | DFDNRIYQMELQRYDLQKERLN |
| | | | | | ADISGDVIVRATSDGKIDSLSVTA |
| | | | | | GQMVNTGDSLLQUIPESIDHYLL |
| | | | | | VAWVPNDAAPYIAVGDKVNLR |
| | | | | | YEAFPAEKFGQFSATISQISKTPA |
| | | | | | STQEMMTYPGAPKNVQGTSIPY |
| | | | | | YKVVIKPDREVVEYDGKRISLEN |
| | | | | | GMKAQSTLFLEKRRIYQWMLSP |
| | | | | | FYDMKHSATGPVNE |
| 3 | WP_007375114.1 | HlyD family | Kosakonia | 280 | MFRKEALENRKMRWRGRAILLP |
| | | secretion | radicincitans | | GVPLWLIIMFCVCFLAAFLTFVID |
| | | protein | | | GTYMRRVNVSGEITTWPRPVTIY |
| | | | | | SNAQGFIVKSYVTEGQKIKKGDP |
| | | | | | VYLIDVSRSTHSGVVSDNQRNDI |
| | | | | | EQQITRVENIIAGLQESKNATLKT |
| | | | | | LEKQKQQYSEALKFSTAVIRRAE |
| | | | | | EGVKLMKENMENYRRYQARGLI |
| | | | | | TKDQLTNHVTLYYQQQNDLLGL |
| | | | | | RSQSEQNALQVTSLESEIQTQAA |
| | | | | | DFDNRIYQMELQRYDLQKERLN |
| | | | | | ADISGDVIVRATSDGKIDSLSVTA |
| | | | | | GQMVNAGDSLLQUIPESIDHHLL |
| | | | | | VAWVPNDATPYIAVGDKVNLRY |
| | | | | | EAFPAEKFGQFSATISQISKTPAS |
| | | | | | TQEMMTYPGAPKNLQGTSIPYY |
| | | | | | KVVIKPDREVVEYDGKRISLENG |
| | | | | | MKAQSTLFLEKRRIYQWMLSPF |
| | | | | | YDMKHSATGPVNE |
| 3 | WP_108901371.1 | HlyD family | Limnobaculum | 281 | MDKRRLFRQEAIEHQKAKWAGT |
| | | secretion | parvum | | ALLITGYPAWIIACASAFFVIVLII |
| | | protein | | | SLTFGNYTRRINVNGEIVTQPRAI |
| | | | | | NIFAPQQGFISKLLVDAGVEVTK |
| | | | | | GTALYQLDVSRIARSGNVTIHTT |
| | | | | | EAIEKQLVHVEGIIAKLEKNKQA |
| | | | | | TQENIKQQIAQSEDANKKSQSLV |
| | | | | | NNAAKGVEEMRQSMKNYEEYQ |
| | | | | | RQGLVNKEQFNNQRYLYYQQQS |
| | | | | | SYQALNTQIIQENLKISNLQSELT |
| | | | | | TSAADFDNQIAQYQYQLNELQR |
| | | | | | QLTEADAAGMLIINAPSDGKIES |
| | | | | | LSVTEGQMVNPGDSLAQLIPTSI |
| | | | | | NAYYLVLWLPNNSLPYVSPGDRI |
| | | | | | NIRYDAFPYQKFGQFPGTVQTVS |
| | | | | | AVPASIQEMSTYNSAPRAAGGG |
| | | | | | TDQSYYKVIVALDSHHFMLANK |
| | | | | | ELRLTSGMKAQTTLFLEKRPLYQ |
| | | | | | WMLSPFYDIKKSITGPVNG |
| 3 | WP_015422938.1 | HlyD family | Morganella | 282 | MFRKAALDNRKMKWRGKALLL |
| | | secretion | morganii | | PGIPLWLIIVCSFLFLMVFLTFILS |
| | | protein | | | GSYTRRVSVTGEITTNPRAVTVY |
| | | | | | AGVQGFITGQYVNEGQIIRKGDP |
| | | | | | VYRIDTSRATASGVVSENQRREI |
| | | | | | ENQLQHIRDMTARLRDNKQTTL |
| | | | | | ALLEKQKSLYAQAQLRSAGIIER |
| | | | | | AQEGIRIMKQNMDNYHHYRESG |
| | | | | | LINKDQWLNQAALYYSQQNSLL |
| | | | | | GLSGQNEQNILQVTSLESQILTQ |
| | | | | | AAEFDNRIYQLELQQSELMKEQ |
| | | | | | VNNDAGGEIIVRALTDGRVDSLG |
| | | | | | VTVGQMVNPGDTLLQILPENIRQ |
| | | | | | YWLALWVPNDALPYLNVGDAV |
| | | | | | NIRYEAFPAEKFGQFAATIQVISK |
| | | | | | TPASAQEMMTYAGAPKVSQMP |
| | | | | | QSIPYYKVIVRPEKQMIAYDGRE |
| | | | | | LSLQNGMKANTTLFLEKRKIYQ |
| | | | | | WMVSPFYDMKQSASGPTGADTP |
| 3 | WP_036412656.1 | HlyD family | Morganella | 283 | MFRKAALDNRKMKWRGKALLL |
| | | secretion | morganii | | PGIPLWLIIVCSFLFLMVFLTLILS |
| | | protein | | | GSYTRRVSVTGEITTNPRAVTVY |
| | | | | | AGVQGFITGQYVNEGQIIRKGDP |
| | | | | | VYRIDTSRATASGVVSENQRREI |
| | | | | | ENQLQHIRDMTARLRDNKQTTL |
| | | | | | ASLEKQKSLYAQAQLRSAGIIER |
| | | | | | AQEGIRIMKQNMDNYHHYRERG |
| | | | | | LINKDQWLNQAALYYSQQNSLL |
| | | | | | GLSGQNEQNILQVTSLESQILTQ |
| | | | | | AAEFDNRIYQLELQQSELMKEQ |
| | | | | | VNNDAGGEIIVCALTDGRVDSLG |
| | | | | | VTVGQMVNPGDTLLQILPENIRQ |
| | | | | | YWLALWVPNDALPYLNVGDAV |
| | | | | | NIRYEAFPAEKFGQFAATIQVISK |
| | | | | | TPASAQEMMTYAGAPKVSQMP |
| | | | | | QSIPYYKVIVRPEKQMIAYDGRE |
| | | | | | LSLQNGMKANTTLFLEKRKIYQ |
| | | | | | WMVSPFYDMKQSASGPTGADTP |
| 3 | WP_013404450.1 | HlyD family | Photobacterium | 284 | MFRKEAKLSTNWTGEAILLKGIN |
| | | secretion | damselae | | PIIILFFSIIFISALILFIIVFSYTH |
| | | protein | | | RMMVVGEISSSPRPTWIYSNFTGIIS |
| | | | | | ERFVQEGDSIKKGASIYKIDVSKK |
| | | | | | NNIGIVSDNRKRTILIQINKINEIN |
| | | | | | ENLKENYDRNTRIIQNKLKQYQE |
| | | | | | SIKFLSLEIKNNKITINEFKNNMIN |
| | | | | | YKSYQKKGLINKDQLISQILNYN |
| | | | | | QQQSTLTNLIQQENQYKLQVTD |
| | | | | | LNGELVSNKLKLDNELNNNELKI |
| | | | | | FDLNKEPINNSSEQSIIIRSPINGV |
| | | | | | VDSLSINHGQMISVRDSLVQIIPN |
| | | | | | DISDYYLVLWVSNKIIPYIKKGDP |
| | | | | | VYIKYDAFPAEKFGQFKGTIFDIS |
| | | | | | HIPATINEMNSYQSSPKLGSTND |
| | | | | | RAIF |
| 3 | WP_033633129.1 | MULTISPECIES: | Serratia | 285 | MRDLFRQEATDHQRAKWAGKA |
| | | HlyD | | | LLINGLPAWCFGLLSFLFILVFLS |
| | | family | | | FLIFSHYTRRINVYGEITTFPRSV |
| | | secretion | | | NVFAPQQGFISERFVEVGDAVKK |
| | | protein | | | GQRLYQIDVSRVTDNGKVSANT |
| | | | | | RLALENQLKHVDSIILKLQDNKR |
| | | | | | MTLENLRAQKKQYELAHMQSK |
| | | | | | QLLDNAREGVAFAQDNMRSYK |
| | | | | | EYQQRGLITKDQLSGQTYSFYQQ |
| | | | | | QSLFQNLHSQHIQESLQITNLESD |
| | | | | | IVTRAADFDNQISQYEFQRNDLQ |
| | | | | | RQLAEADASGALIVNAPSDGRIE |
| | | | | | SLSVTPGQMVNSGDSLVQIIPRN |
| | | | | | GAIYQLVLWLPNTSVPYVSAGD |
| | | | | | GINIRYDAFPYEKFGQFPGRIESI |
| | | | | | AYVPASIQEMSTYSSSPVHQPSV |
| | | | | | QTASFYKVTVSLDETHISYQGKA |
| | | | | | LQLTNGMRAQSTLFLEKRPLYQ |
| | | | | | WMFSPFYDMKNSLMGPIND |
| 3 | WP_033648236.1 | MULTISPECIES: | Serratia | 286 | MRDLFRQEATDHQRAKWAGKA |
| | | HlyD | | | LLINGLPAWCFGLLSFLFILVFLS |
| | | family | | | FLIFSHYTRRINVYGEITTFPRSV |
| | | secretion | | | NVFAPQQGFISERFVEVGDVVKK |
| | | protein | | | GQRLYQIDVSRVTDNGKVSANT |
| | | | | | RLALENQLKHVDSIILKLQDNKR |
| | | | | | MTLENLRAQKKQYELAHMQSK |
| | | | | | QLLDNAREGVAFAQDNMRSYK |
| | | | | | EYQQRGLITKDQLSGQTYSFYQQ |
| | | | | | QSLFQNLHSQHIQESLQITNLESD |
| | | | | | IVTRAADFDNQISQYQFQRNDLQ |
| | | | | | RQLAEADASGALIVNAPSDGRIE |
| | | | | | SLSVTPGQMVNSGDSLVQIIPRN |
| | | | | | GAIYQLVLWLPNASVPYVSAGD |
| | | | | | AINIRYDAFPYEKFGQFPGRIESI |
| | | | | | AYVPASMQEMSTYSSSPVHQPN |
| | | | | | AQTASYYKVTVSLDETHISYQG |
| | | | | | KALQLTNGMRAQSTLFLEKRPL |
| | | | | | YQWMFSPFYDMKNSLMGPIND |
| 3 | WP_047567594.1 | HlyD family | Serratia | 287 | MFRQEAIDNQKMKWRGRAILLP |
| | | secretion | marcescens | | GIPFWLTAGLCLFFLIAFLTFAIA |
| | | protein | | | GTYTRRVNVTGEISTYPRAANV |
| | | | | | YSAVQGVVVKQFVTEGQVIAAG |
| | | | | | APIYQIDVSKSTRSGVVSDNQQR |
| | | | | | DIDGQLARIAQIISRLESSKQATL |
| | | | | | TMLEQQKAQYTAAFTRSTDILK |
| | | | | | RAQEGIRIMKENMENYRHYQTK |
| | | | | | GLITKDQLTNQVALYYQQQNNL |
| | | | | | LGLSGQNEQNALQITALESQIHIQ |
| | | | | | AANFDNQIYQMELQRHELQKER |
| | | | | | LNIDAGGAIIVRALADGRIDSLSV |
| | | | | | TVGQMVNVGDSLLQVIPHNIAH |
| | | | | | YALVLWVPNDAIPYIATGDKVN |
| | | | | | VRYDAFPAEKFGQFAATVSVISK |
| | | | | | APASPQEMLTYQGAPKAALTAA |
| | | | | | VPYYKTIVIPEKQVIAYDGKRLS |
| | | | | | LENGMKAQSTLFLEKRQIYQWM |
| | | | | | LSPFYDMKHSATGPINE |
| 3 | WP_060559629.1 | HlyD family | Serratia | 288 | MFRQEAIDNQKMKWRGRALLLP |
| | | secretion | marcescens | | GIPFWFTAGLCLFFLIAFLTFTIAG |
| | | protein | | | TYTRRVNVTGEISTYPRAANVYS |
| | | | | | TVQGIVVKQFVTEGQVIAVGAPI |
| | | | | | YQIDVSKSTRSGVLSDNQRRDID |
| | | | | | GQLARIAQIINRLESSKQATLIML |
| | | | | | EQQKAQYTAAFTRSTDILKRAQE |
| | | | | | GIRIMKENMENYRHYQTKGLIN |
| | | | | | KDQLTNQVALYYQQQNNLLGLS |
| | | | | | GQNEQNALQITALESQIHIQAAE |
| | | | | | FDNQIYQMELQRYELQKERQNI |
| | | | | | DATGDIIVRALAGGRIDSLSVTV |
| | | | | | GQMVNIGDSLLQIIPQNIDRYAL |
| | | | | | VLWVPNDAIPYIAAGDKVNVRY |
| | | | | | DAFPAEKFGQFAGTVSVISKAPA |
| | | | | | SPQEMLTYQGAPKAALTAAVPY |
| | | | | | YKVIVIPEKQSIAYDGKRLSLEN |
| | | | | | GMKAQSTLFLEKRKMYQWMLS |
| | | | | | PFYDMKHSATGPVNE |
| 3 | WP_149560094.1 | HlyD family | Serratia | 289 | MFRQEAIDNQKMKWRGRALLLP |
| | | secretion | marcescens | | GIPFWLTAGLCLFFLIAFLTFAIA |
| | | protein | | | GTYTRRVNVTGEISTYPRAANV |
| | | | | | YSAVQGVVVKQFVTEGQVIAAG |
| | | | | | APIYQIDVSKSTRSGVVSDNQQR |
| | | | | | NIDGQLARIAQIISRLESSKQATL |
| | | | | | TMLEKQKAQYIAAFTRSTDILKR |
| | | | | | AQEGIRIMKENMENYRHYQTKG |
| | | | | | LITKDQLTNQVALYYQQQNNLL |
| | | | | | GLSGQNEQNALQISALESQIHIQA |
| | | | | | ANFDNQIYQMELQRHELQKERL |
| | | | | | NIDAGGAIIVRALADGRIDSLSVT |
| | | | | | VGQMVNVGDSLLQVIPHNIDHY |
| | | | | | ALVLWVPNDAIPYIAAGDKVNV |
| | | | | | RYDAFPAEKFGQFAGTVAVISKA |
| | | | | | PASPQEMLTYQGAPKAALTAAV |
| | | | | | PYYKVIVIPEKQFIAYDGKRLSLE |
| | | | | | NGMKAQSTLFLEKRKIYQWMLS |
| | | | | | PFYDMKHSATGPVNE |
| 3 | WP_041417051.1 | HlyD family | Serratia | 290 | MFRQEAIDNQKMKWRGRAILLP |
| | | secretion | plymuthica | | GIPFWAMAGLCFFFIVAFLSFIIA |
| | | protein | | | GTYTRRVNVTGEISTYPRAANV |
| | | | | | YSGVQGVVVKQFVTEGQEIKSG |
| | | | | | DPIYQIDVSKSTRSGVVSDNKRQ |
| | | | | | DIDNQLERITQIISRLESNKKATL |
| | | | | | DMLEKQRAQYTAAFQRSTDILR |
| | | | | | RAQEGIHIMKENMENYRRYQTR |
| | | | | | GLINKDQLSDQVALYYQQQNNL |
| | | | | | LGLSSQNEQNALQITALESQIHT |
| | | | | | QAAEFDNQIYQMELQRYDLQKE |
| | | | | | MLNIDADGTIIVRVLADGRVDSL |
| | | | | | SVTVGQMVNVGDSLLQIIPHKID |
| | | | | | HYALVLWVPNDAIPYIATGDKV |
| | | | | | NVRYDAFPAEKFGQFAGTVSVIS |
| | | | | | KAPASPQEMLTYQGAPKAALTT |
| | | | | | AVPYYKVIVRPEKQVIVYNRKRL |
| | | | | | SLENGMKAQSTLFLEKRSIYQW |
| | | | | | MLSPFYDMKHSATGPVNE |
| 3 | WP_063919749.1 | HlyD family | Serratia | 291 | MFRQEAIGNQKLKWRGRALLLP |
| | | secretion | surfactant- | | GIPAWFTAGLCLFFLIAFLTFVLA |
| | | protein | faciens | | GTYTRRVNVTGEISTYPRAANV |
| | | | | | YSTVQGVVIKQFVTEGQRIAAGA |
| | | | | | AIYQIDVSKSTRSGVVSDNQQRD |
| | | | | | IDGQLARIAQIISRLESSKQATLT |
| | | | | | MLEKQKAQYTAAFTRSTDILQR |
| | | | | | AQEGIRIMKENMENYRHYQTKG |
| | | | | | LINKDQLTNQVALYYQQQNNLL |
| | | | | | GLSGQNEQNALQITALESQIHTQ |
| | | | | | AAEFDNQIYQMELQRYELQKER |
| | | | | | QNIDATGDIIVRALAGGRIDSLSV |
| | | | | | TVGQMVNVGDSLLQVLPHDIDR |
| | | | | | YALVLWVPNDAIPYIAAGDRVN |
| | | | | | VRYDAFPAEKFGQFAGTVSVISK |
| | | | | | APASPQEMLTYQGAPKAALTAA |
| | | | | | VPYYKVIVIPEKQTIAYDGKRLS |
| | | | | | LENGMKAQSTLFLEKRLVYQW |
| | | | | | MLSPFYDMKHSATGPVNE |
| 3 | WP_002208949.1 | MULTISPECIES: | Yersinia | 292 | MSSNQIFRRDAVEYIRTKWLGK |
| | | HlyD | pseudo- | | ALLTSGYSTTFIAALCAIFLVLLIT |
| | | family | tuberculosis | | LIIYGTYTRRINVNGEVISQPHPIN |
| | | secretion | complex | | IFSPQQGFITKKWVEVGDIVRKG |
| | | protein | | | QHLYQIDVSRTTFSGNVSLNSLE |
| | | | | | AINNQLSQIDSIINNTQKNKELTL |
| | | | | | LNLRQQLAQYQKAHKKSQELVD |
| | | | | | NAGKGMDDMRRTMASYGTYQR |
| | | | | | QGLITKDQLTNQRSLFYQQQNAF |
| | | | | | QSLNTQLIQESLQIAKLESEISTR |
| | | | | | ASDFDNDISQYLFQKGDLKRQL |
| | | | | | AEVDASGMLLINSPSDGKIENMS |
| | | | | | VTQGQMVNVNDSLVQLTPSDNP |
| | | | | | YYCLVLWVPNNSVPYINTGDKV |
| | | | | | NIRYDAFPFEKFGQFPGRIISISNV |
| | | | | | PVSQQEIASYNIAPRLPNGGLIEP |
| | | | | | YYKVIVALDDIHFRYQSKPLMLS |
| | | | | | NGLKANVTLFLEKRPLYQWMLS |
| | | | | | PFYDIKKSVTGPVNE |
| 4 | N_MFP_mcnA | Membrane | Escherichia | 293 | MFRQEATVYRKMKWCGRAVLL |
| | | fusion protein | coli | | PGIPPWLVISVSVFFFISFIVFVME |
| | | | | | GTYTRRINVEGEITTWPRPVNVY |
| | | | | | SGVQGVVIKQFVTEGQRIKKGDP |
| | | | | | IYLIDVSKSTSSGVVGDNKRRDIE |
| | | | | | KQLSRIGDIISRLEENKKTTLQTL |
| | | | | | EKQRIQYYSAFERSTEILRRAEEG |
| | | | | | VKIMKSNMDNYKQYQTKGLINK |
| | | | | | DQLTNQIALYYQQQNNILSLSTQ |
| | | | | | NEQNLLQVTSLESQMQTLAAEF |
| | | | | | DNRIYQVELQRYELQKELVDTD |
| | | | | | AGRDIIIRALSDGKIDSLSVTPGQ |
| | | | | | MVSVGDSLLQUIPEEIKNYHLIVW |
| | | | | | VPNNAIPYISVGDNVNVRYEAFP |
| | | | | | PEKFGQFTAKIMLISRTPASAQE |
| | | | | | MQTYPGAPRNNTGVSVPYYKIV |
| | | | | | LNPEQQTIEYGEKKMPLENGMK |
| | | | | | AQSTLFLEKRKIYQWMLSPFYN |
| | | | | | MKYSAVGPVNEQ |
| 4 | WP_049086513.1 | MULTISPECIES: | Klebsiella | 294 | MKNELYRKEAIEYKRNYWKGK |
| | | HlyD | | | ALLLAGMPSWLIGSLSFLFLICLI |
| | | family | | | LTLIFCNYTQRIDVKGEVITLPHSI |
| | | secretion | | | NVFAPQQGFVIKKHVAVGDIVE |
| | | protein | | | KGTPLYEIDVSRSTINGNVNETM |
| | | | | | TAVITEKIKNANEIINKILKNKTE |
| | | | | | TLDALNIQIKQYELTLQETDKML |
| | | | | | KNAGDGLKKMKDNLSSYDKYL |
| | | | | | KEGLITKDQYNYQHSLYFQQQS |
| | | | | | TYQSLVSQKMQIASQLTQLNSD |
| | | | | | KIIKGADFDNQISTQKNQINDYK |
| | | | | | NQRVESTANGNVVVKATAAGKI |
| | | | | | ESVSVTAGQMVDKGSSLAQIKPT |
| | | | | | GNVEYYLILWLPNNSIPYINPGD |
| | | | | | TVNIRYDAFPSDKFGQFPGKVVS |
| | | | | | ISSVPASRQEMSEYTNAITNPNQ |
| | | | | | QELALYKATIKIKKTNFIYRNKV |
| | | | | | LKLSNGLKAQAIVFLEERPLYM |
| | | | | | WMFTPFYKINQSVSGPINE |
| 4 | WP_015705589.1 | HlyD family | Klebsiella | 295 | MFRKEALENRDKTWCGKAVLLP |
| | | secretion | aerogenes | | GISSRLVIITSALFFVCFIVLISTGT |
| | | protein | | | YTRRISVQGEVTTWPRPVNVYS |
| | | | | | GVQGFVATQFVREGDPVKKGDP |
| | | | | | VYQIDVSKSTRRGVVNETQRQDI |
| | | | | | ENQLKRIAEIIARLRESKETTLRM |
| | | | | | LEQQKIQYSASFERSAGIVRRAE |
| | | | | | EGIKLMKNNMDNYRRYQQQGLI |
| | | | | | NKDQLTSLVALYYQQHNSLLSL |
| | | | | | SAQHEQNALQLTNLESQIQTQAA |
| | | | | | EFDNRVYQMELQRYELQKERIN |
| | | | | | TDAGGDIIIRASSDGKIDSLSVTP |
| | | | | | GQMVSEGDSLLQIIPEKIQRYYL |
| | | | | | VLWVPNEAVPYISVGDRVNIRYE |
| | | | | | AFPAEKFGQFSAKVTLISRTPASY |
| | | | | | REMQTYTTAPINSTAGAAAYYK |
| | | | | | VVISPEKQAVSYAGKRLPLENG |
| | | | | | MKAQSTLFLEKRKIYQWMLSPF |
| | | | | | YDMKRSVTGPIHEP |
| 4 | WP_064563846.1 | HlyD family | Kosakonia | 296 | MGQKLFRQEAVDHQRSYWKGK |
| | | secretion | oryzae | | VILFKGVSPFIISACCIAFMAVLIA |
| | | protein | | | MLCFFKFTQRIDVVGEVITLPHPL |
| | | | | | NISSPQPGFISQQFIQVGDTVKKG |
| | | | | | DVLFELDVSKSTNAGNVSDTNIS |
| | | | | | MIKAKITNAEEIINKLKANKDDFI |
| | | | | | ATTQRQIQRYEYSLTETDGLLAT |
| | | | | | AKQGLTKMESALQNYNQYLKK |
| | | | | | GLINKDQYNNQHSLYVQQQNAF |
| | | | | | QSLSTQKMQLELQLTQSRSDITV |
| | | | | | KSAELDNQIAAQYNQLSDFQSQL |
| | | | | | IETNANGKILVKSTIGGRVESVA |
| | | | | | VTPGQMVDAGSSLAQIKQLTDV |
| | | | | | KYYLLLWLPDNSLPYVKIGDTV |
| | | | | | NIRYDAFPSDKFGQFSGKIKSISS |
| | | | | | MPATRRELAEYSIGQSNTNSAQT |
| | | | | | LYKAIIDIKQKEFIYKDKKLELSN |
| | | | | | GLKAKSLVFMEERPLYQWLFSP |
| | | | | | VYKVAQSLTGPNNE |
| 4 | WP_081120700.1 | HlyD family | Kosakonia | 297 | MGQKLFRQEAVDHQRSYWKGK |
| | | secretion | radicincitans | | VILFKGVSPFIISACCIAFMAILIV |
| | | protein | | | MLCFFKFTQRIDVVGEVITLPHPL |
| | | | | | NISSPQMGFISQQFIQVGDTVKK |
| | | | | | GDMLFELDVSKSTNAGNVSDTN |
| | | | | | ISMIKAKIANAEEIINKLKVNKDD |
| | | | | | FIATTKRQIQRYERSLAETDGLLT |
| | | | | | TAKQGLTKMEGALQNYNQYLK |
| | | | | | KGLINKDQYNNQHSLYVQQQNA |
| | | | | | FQSLSTQKMQLELQLTQSRSDIT |
| | | | | | VKSAELDNQIASQYNQLSDFQSQ |
| | | | | | LIETNANGKILVKSTIAGRVESIA |
| | | | | | VTPGQMVDAGSSLAQIKQLTDIK |
| | | | | | YYLLLWLPDNSLPYVKIGDTVNI |
| | | | | | RYDAFPSDKFGQFSGKIKSISSIP |
| | | | | | ATRRELSEYSAGQTNTNSQLTLY |
| | | | | | KAIIDIKENEFIYKNKKLALSNGL |
| | | | | | KAKSIVFMEERPFYQWLFSPAYK |
| | | | | | VAQSLTGPKNE |
| 4 | WP_013508681.1 | HlyD family | Pantoea sp. | 298 | MLFRKEVSEHLQSRYAGPILLIT |
| | | secretion | At-9b | | GWPVWITLCITLLFLAALLLFLW |
| | | protein | | | QGSYTRRTSVSGEVITWPHTINL |
| | | | | | FAPEQGVISRLLVSPGQQVSAGT |
| | | | | | PLYALDISRVSASGNLSATTRAL |
| | | | | | LKKQQQQMGEMLHQLEQNRRA |
| | | | | | TLASIQQQLDQLRSAREKTQQM |
| | | | | | VDSATQGLDVMRSSMNDYNRF |
| | | | | | ARRGLITSDQQNNQRYLFYQQLS |
| | | | | | VWHSLNSQRVQQDMQIAELLSQ |
| | | | | | QVTRAADFDSQLAQYNIRQADIE |
| | | | | | RQLAEADAGSERLIAAPSAGRISS |
| | | | | | LSVTAGEMVSDGDSLAQLVPAG |
| | | | | | DDIPSLVIWLPNDSIPYVKPGDVI |
| | | | | | NVSYAAYPAEKYGQFPGKVLSIS |
| | | | | | SAPVPLRELNNYGSAPRSPAGQI |
| | | | | | TGAWFKANVALNHQGLRWQGK |
| | | | | | DLPLASGMQLQATVFLEKRPLW |
| | | | | | QWMLSPYYFLKNSITGPVSDQR |
| 4 | WP_112152215.1 | MULTISPECIES: | Rahnella | 299 | MCCYLSKILFLQPVFLEHLNMLF |
| | | HlyD | | | RKEVNEHQQSHWAGKALLLAG |
| | | family | | | WPLWIVTTLTALFLFALLVFLIFA |
| | | secretion | | | NYTRRINVSGEIITQPHSINLFSPE |
| | | protein | | | QGVVTKLYVDTGKPVKKGQPLY |
| | | | | | EIDVSRVTQAGNVSTTTLAAIKK |
| | | | | | QREQIDSIIAQLEHNKQETLQNL |
| | | | | | QQQLDQYQKAHEVSQSMVDSA |
| | | | | | QEGLDAMKKSMQNYGAYQKKG |
| | | | | | LINTDQLNNQRYLFYQQQTSFQS |
| | | | | | LNTQAIQEALQITNLRSELVTRA |
| | | | | | AEFDNQISQYGYQRNDLERQLA |
| | | | | | QADAKGSLLITAPTSGKISSLSVT |
| | | | | | PGQMVNAGDSLAQLVPASNSPF |
| | | | | | FLVAWLPNESVPYVKAGEHINIR |
| | | | | | YEAYPFEKYGQFPGKVESISSAP |
| | | | | | VSEQELNSYASAPRTANGTISGP |
| | | | | | YYKVIVSLDKKAMNWHGETLN |
| | | | | | LSSGMKAESTLFLEKRPLYQWM |
| | | | | | LSPYYSMKKSVVGPINESR |
| 4 | WP_112197132.1 | MULTISPECIES: | Rahnella | 300 | MCCYLSKILFLQPVFLERLNMLF |
| | | HlyD | | | RKEVNEHQQSHWAGKALLLSG |
| | | family | | | WPLWIVTTLTALFLFALLIFLIFA |
| | | secretion | | | NYTRRINVSGEIITQPHSINLFSPE |
| | | protein | | | QGVVTKLYVDTGKPVKKGQPLY |
| | | | | | EIDVSRVTQAGNVSTTTLAAIKK |
| | | | | | QREQIDSIIAQLEHNKQETLQNL |
| | | | | | QQQLDQYQKAHEVSQSMVDSA |
| | | | | | QEGLDAMKKSMQNYGAYQKKG |
| | | | | | LINTDQLNNQRYLFYQQQTSFQS |
| | | | | | LNTQAIQEALQITNLRSELVTRA |
| | | | | | AEFDNQISQYGYQRNDLERQLA |
| | | | | | QADAKGSLLITAPTSGKISSLSVT |
| | | | | | PGQMVNAGDSLAQLVPASNSPF |
| | | | | | FLVAWLPNESVPYVKAGEHINIR |
| | | | | | YEAYPFEKYGQFPGKVESISSAP |
| | | | | | VSEQELNSYASAPRTANGTISGP |
| | | | | | YYKVIVSLDKKAMNWHGETLN |
| | | | | | LSSGMKAESTLFLEKRPLYQWM |
| | | | | | LSPYYSMKKSVVGPINESR |
| 4 | WP_032716441.1 | MULTISPECIES: | Raoultella | 301 | MGKRLFRREAIEHQRSYWKGRV |
| | | HlyD | | | ILLRGVSPLIISACCVAFLVMIIA |
| | | family | | | MLCLFKYTKRIDVVGEVITLPHP |
| | | secretion | | | LNISSPQPGSVSRQSIQVGDVVK |
| | | protein | | | KGEVLFELDVSKSTDGGNVSDT |
| | | | | | NVAMVKAKIVNAEDIIAKLKVN |
| | | | | | KDGFIETTHRQILRYEHSLNETEK |
| | | | | | MLTTAQKGLTHIEKSLQNYSDY |
| | | | | | LKRGLINKDQYNNQHSLFVQQQ |
| | | | | | NAFQSLSSQKMQLELQLTQTRSD |
| | | | | | LTVKTAELDNQITSQYSQLRDLQ |
| | | | | | TQLIENSANGKIVVKSTIDGRVES |
| | | | | | IAVTRGQMVEAGSSLAQVKQLT |
| | | | | | GIEYYLLLWLPDNSLPYVKIGDI |
| | | | | | VNIRYDAFPSDKFGQFSGKVLSIS |
| | | | | | SMPATRRELAEYSNNQMSNAPA |
| | | | | | PTLYKAIINIKDKEFIYKDRRLVL |
| | | | | | ANGLKAKSIVFTEERPLYQWLFS |
| | | | | | PVYKVVQSLKGPGDE |
| 4 | WP_015378377.1 | MULTISPECIES: | Serratia | 302 | MSDNKSRNIFRQEAVNYQRQRW |
| | | HlyD | | | TGRALLISGASSHVIAATCFTFILI |
| | | family | | | LAGALYFCEYTRRVDVEGEVISI |
| | | secretion | | | PHSTNVFSSQYGYVSQAFHKAG |
| | | protein | | | DVVEKDEPLYVIDVSRSTTSGNV |
| | | | | | SHIAASEISKQISNLDSIISKLNKN |
| | | | | | KTAMLDNLRKQINSYENAYKET |
| | | | | | EKLNLSAKEGMDKMRDSLKNY |
| | | | | | EHYLTKGLITKDQLNYQRSLFQQ |
| | | | | | QQSSYQALYSQKIQQNIQLSQLR |
| | | | | | SDLLIRAADYDNQISQSESQRSD |
| | | | | | LQRQLAESNAGGQVIIKAPLKGK |
| | | | | | IESLSVTAGQVVESGSSLAQIKPI |
| | | | | | DNIKYYLVLWLPNNSLPYVKIGD |
| | | | | | GINIRYEAFPSDKFGQFPGKIESIS |
| | | | | | SVPASPQEMSEYSSGNKRERNA |
| | | | | | YYKVLASISDTQFSDKGKYLEISS |
| | | | | | GLQARVIVFLDKKPLYQWAVAP |
| | | | | | LYDIKNSVVGRVDEGQ |
| 4 | WP_019453449.1 | MULTISPECIES: | Serratia | 303 | MSENKSRNIFRQEAVNYQRQRW |
| | | HlyD | | | TGRALLISGASSYVIAVTCFIFILI |
| | | family | | | LAGALYFCEYTRRVDVEGEVISI |
| | | secretion | | | PHSTNVFSSQYGYVNRSFHQAG |
| | | protein | | | DVVEKDEPLYVIDVSRSTASGNV |
| | | | | | SHIAASEINKQIGNLDSIISKLNKN |
| | | | | | KDAMLDNLRKQINSYELAYKET |
| | | | | | EKLNLNARDGMEKMRESLKNY |
| | | | | | EHYLTKGLITKDQLNYQRSLFQQ |
| | | | | | QQSSYQALNSQKIQQNIQLSQLR |
| | | | | | SDLLIRAADYDNQISQSESQRSEL |
| | | | | | QRQLAESNAGDQVIIKAPLKGKI |
| | | | | | ESLSVTAGQVVESGSSLAQIKPIE |
| | | | | | NIKYYLVLWLPNNSLPYVKIGDG |
| | | | | | INIRYEAFPSDKFGQFPGRIESISS |
| | | | | | VPASPQEMSEYSSGNKRERNAY |
| | | | | | YKVLASISDTQFSDKGKYLEISSG |
| | | | | | LQARVIVFLDKKPLYQWAVAPL |
| | | | | | YDIKNSVVGRVDEGS |
| 5 | PDI_MFP_mcpD | HlyD family | Escherichia | 304 | MNIFRSEAIEHHNDTEYGDIILPT |
| | | efflux | coli | | SFSLSVCATVTLFIMLSLTVFIYY |
| | | transporter | | | GSYTRKAHLTGIVMPSSGLVKIIP |
| | | periplasmic | | | QYAGYVTQLTVSEGEHVTAGTQ |
| | | adaptor | | | LYHISGEHYNGNGTGTLATMSIS |
| | | subunit | | | LKTQYIMLASQQSFESRDNSQQQ |
| | | | | | EAIRQRMISLEPQIRSAEQRLQLA |
| | | | | | ERQAELAISVMERYKKLAGTHY |
| | | | | | VSDIEFQQKQIDVSAAQQNVEDQ |
| | | | | | RQGLLQLHTAMDTAKDELNHLI |
| | | | | | VQGKSRKAELDRQLQVLKQQQ |
| | | | | | DELAGQEKFTLRAPVSGTIAAVL |
| | | | | | IKQGQSVKASEPVMTLIPDNAHL |
| | | | | | QIELYATSQKAGFIRPGQRVSLK |
| | | | | | FSAFPYQKFGIQYGTIRKISHTTL |
| | | | | | APSDLLPVSPVTWKENEGHYRVI |
| | | | | | VEPENTFIFAYGKKEPLRPGMTL |
| | | | | | EGDVNLDTRHLWEWLTEPLWS |
| | | | | | MKGNL |
| 5 | WP_001685252.1 | MULTISPECIES: | Entero- | 305 | MNIFRSEAIEHHNDTEYGDIILPT |
| | | HlyD | bacteriaceae | | SFSLSVCATVTLFIMLSLTVFIYY |
| | | family efflux | | | GSYTRKAHLTGIVMPSSGLVKIIP |
| | | transporter | | | QYAGYVTQLTVSEGEHVTAGTQ |
| | | periplasmic | | | LYHISGEHYNGNGTGTLATMSIS |
| | | adaptor | | | LKTQSIMLASQQSFESRDNSQQQ |
| | | subunit | | | EAIRQRMVSLEPQIRSAEQRLQL |
| | | | | | AERQAELAISVMERYKKLAGTH |
| | | | | | YVSDIEFQQKQIDVSAAQQNVED |
| | | | | | QRQGLLQLHTAMDTAKDELNHL |
| | | | | | IVQGKSRKAELDRQLQVLKQQQ |
| | | | | | DELAGQEKFTLRAPVSGTIAAVL |
| | | | | | IKQGQSVKASEPVMTLIPDNAHL |
| | | | | | QIELYATSQKAGFIRPGQRVSLK |
| | | | | | FSAFPYQKFGIQYGTIREISHTTL |
| | | | | | APSDLLPVSPVTWKENEGHYRVI |
| | | | | | VEPENTFIFAYGKKEPLRPGMTL |
| | | | | | EGDVNLDTRHLWEWLTEPLWS |
| | | | | | MKGNL |
| 5 | WP_015059309.1 | MULTISPECIES: | Entero- | 306 | MNIFRSEAIEHHNDTEYGDIILPT |
| | | HlyD | bacteriaceae | | SFSLSVCATVTLFIMLSLTVFIYY |
| | | family efflux | | | GSYTRKAHLTGIVMPSSGLVKIIP |
| | | transporter | | | QYAGYVTQLTVSEGEHVTAGTQ |
| | | periplasmic | | | LYHISGEHYNGNGTGTLATMSIS |
| | | adaptor | | | LKTQYIMLASQQSFESRDNSQQQ |
| | | subunit | | | EAIRQRMISLEPQIRSAEQRLQLA |
| | | | | | ERQAELAISVMERYKKLAGTHY |
| | | | | | VSDIEFQQKQIDVSAAQQNVEDQ |
| | | | | | RQGLLQLHTAMDTAKDELNHLI |
| | | | | | VQGKSRKAELDRQLQVLKQQQ |
| | | | | | DELAGQEKFTLRAPVSGTIAAVL |
| | | | | | IKQGQSVKASEPVMTLIPDNAHL |
| | | | | | QIELYATSQKAGFIRPGQRVSLK |
| | | | | | FSAFPYQKFGIQYGTIRKISHTTL |
| | | | | | APSDLLPVSPVTWKENEGHYRVI |
| | | | | | VEPENTFIFAYGKKEPLRPGMTL |
| | | | | | EGDVNLDTRHLWEWLTEPLWS |
| | | | | | MKGNL |
| 5 | WP_063320175.1 | HlyD family | Salmonella | 307 | MNIFRSEAIEHHNDTEYGDIILPT |
| | | efflux | enterica | | SFSLSVCATVTLFIMLSLTVFIYY |
| | | transporter | | | GSYTRKAHLTGIVMPSSGLVKIIP |
| | | periplasmic | | | QYAGYVTQLTVSEGEHVTAGTQ |
| | | adaptor | | | LYHISGEHYNGNGTGTLATMSIS |
| | | subunit | | | LKTQSIMLASQQSFESRDNSQQQ |
| | | | | | EAIRQRMVSLEPQIRSAEQRLQL |
| | | | | | AERQAELAISVMERYKKLAGTH |
| | | | | | YVSDIEFQQKQIDVSAAQQNVED |
| | | | | | QRQGLLQLHTAMDTAKDELNHL |
| | | | | | IVQGKSRKAELDRQLQVLKQQQ |
| | | | | | DELAGQEKFTLRAPVSGTIAAVL |
| | | | | | IKQGQSVKASEPVMTLIPDNAHL |
| | | | | | QIELYATSQKAGFIRPGQRVSLK |
| | | | | | FSAFPYQKFGIQYGTIREISHTTL |
| | | | | | VPSDLLPVSPVTWKENEGHYRVI |
| | | | | | VEPENTFIFAYGKKEPLRPGMTL |
| | | | | | EGDVNLDTRHLWEWLTEPLWS |
| | | | | | MKGNL |
| 6 | S_MFP_mcsA | HlyD family | Escherichia | 308 | MSIFREEALEHNSDTEYGDIVLP |
| | | efflux | coli | | ASFGMSVCAVATLLIFLSVSLFV |
| | | transporter | | | YYGSYTRKAHLTGIVMPSSGLV |
| | | periplasmic | | | KITPQYAGYVTRQTVSEGQHVA |
| | | adaptor | | | AGEPVYHISGEHYNGQGTGTLA |
| | | subunit | | | AMSMSLKTQYAMLSSQQTLELR |
| | | | | | DNSQQQQAILQRIASLKPQIKSAE |
| | | | | | LRLSLAGHQATLAVSVMERYKK |
| | | | | | LAGTHYVSDIEYQQKQIDVSTSL |
| | | | | | QNVEDQRQGLLQLHTAMEAAEE |
| | | | | | DLNHLIVQGESRKAELDRQLQGI |
| | | | | | RQQQDELAGQENFTLTSPVSGTV |
| | | | | | AAVLIRQGQSVRASEPVMTLVP |
| | | | | | DNARLQIELYATSQNAGFIQPGQ |
| | | | | | RVALRFAAFPYQKFGVQYGTIRE |
| | | | | | ISRTTLTPSDLLSVSPVTWKENEG |
| | | | | | HYRVIVEPENTFILAYGKQEPLRP |
| | | | | | GMTLEGDVSLDTRHLWEWLTEP |
| | | | | | LWSLKGKL |
| 6 | WP_061076566.1 | HlyD family | Citrobacter | 309 | MKLFRKEVIENLNSTTYGGVIIPT |
| | | efflux | amalonaticus | | SFSLLLSTIATIALIVLISIFLGFGE |
| | | transporter | | | YTRKARLTGIVMPSSGLIKITPQY |
| | | periplasmic | | | SGYVTKLTIQEGEHVQKGQALY |
| | | adaptor | | | HISGEHYGSKGTGTLAAMNLSL |
| | | subunit | | | QTQYSMLSSQKMLEQRDNNQQ |
| | | | | | QQSIQQHITLIQLQIKSGEQRLLL |
| | | | | | AKHQADLSSLVVRRYQKLINQK |
| | | | | | YVSEIEYQLKQIESASAKENVEN |
| | | | | | QRQLLLQLRASLDNARDELAHLI |
| | | | | | VQGESRNAELSRQLQVIKQQQFE |
| | | | | | LASQESFTLTAPVAGTVAAILIRQ |
| | | | | | GQSVKAQEPVMSLVPENSDLQV |
| | | | | | ELYATSQNAGFIQPGQKVALRYT |
| | | | | | AFPYQKFGVQYGTILEISRITLAP |
| | | | | | TDLLSISPMIWKVNEGHYRVIVK |
| | | | | | PELSHILAYGKYEPLRPGMTLEG |
| | | | | | DVNLDTRYLWEWLTEPLWSLK |
| | | | | | GKI |
| 6 | WP_047726556.1 | MULTISPECIES: | Enterobacter | 310 | MNIFREEALNHQTDSDYGEIVLP |
| | | HlyD | | | ASFGLSVSALTTLFIFLCLILFILY |
| | | family efflux | | | GSYTRKAHLSGIVMPSSGLVKIT |
| | | transporter | | | PQYAGYVTKLTASEGQHIIAGET |
| | | periplasmic | | | LYHISGEHFNEQGAGTLAAIRLS |
| | | adaptor | | | LQTQFTMLTSQQTLELRDNSQQ |
| | | subunit | | | QHAIQQRIVSLKPQVKSAEKRLS |
| | | | | | LAVHQAALAVSVMERYKKLAD |
| | | | | | THYVSDIEYQQKQIDVSTSQQNV |
| | | | | | EDQRQGLLQLHTAMEAAKDDL |
| | | | | | DHLIVQGESRKAEMDRQLQVIR |
| | | | | | QQQDELAGKENFTLSSPVSGTVA |
| | | | | | AVLVRQGQSVRASEPVMTLIPD |
| | | | | | NARLQIELYATSQNAGFIQAGQR |
| | | | | | VALRFSAFPYQKFGVQYGTIREIS |
| | | | | | HTTLTSSDLLSVSPVTWKENEGH |
| | | | | | YRVIVEPENTFILAYGKQEALRP |
| | | | | | GMVLEGDVSLDTRNLWEWLTEP |
| | | | | | LWSLKGKL |
| 6 | WP_032671190.1 | MULTISPECIES: | Enterobacter | 311 | MNIFREEALNHQTDSDYGEIVLP |
| | | HlyD | cloacae | | ASFGLSVSALTTLFIFLCLILFILY |
| | | family efflux | complex | | GSYTRKAHLSGIVMPSSGLVKIT |
| | | transporter | | | PQYAGYVTKLTASEGQHIIAGET |
| | | periplasmic | | | LYHISGEHFNEQGAGTLAAIRLS |
| | | adaptor | | | LQTQFTMLTSQQTLELRDNSQQ |
| | | subunit | | | QHAIQQRIVSLKPQVESAEKRLS |
| | | | | | LAVHQAALAVSVMERYKKLAD |
| | | | | | THYVSDIEYQQKQIDVSTSQQNV |
| | | | | | EDQRQGLLQLHTAMEAAKDDL |
| | | | | | DHLIVQGESRKAEMDRQLQVIR |
| | | | | | QQQDELAGKENFTLTSPVSGTV |
| | | | | | AAVLVRQGQSVRASEPVMTLIP |
| | | | | | DNARLQIELYATSQNAGFIQAGQ |
| | | | | | RVALRFSAFPYQKFGVQYGTIRE |
| | | | | | ISHTTLTSSDLLSVSPVTWKENE |
| | | | | | GHYRVIVEPENTFILAYGKQEAL |
| | | | | | RPGMVLEGDVSLDTRNLWEWL |
| | | | | | TEPLWSLKGKL |
| 6 | WP_040107998.1 | HlyD family | Klebsiella | 312 | MGVFRSEALEYHSDTEYGDVVIP |
| | | efflux | variicola | | ASLSMTACAVATLFIFICVVLFV |
| | | transporter | | | YYGSYTRKAHLTGIVMPSSGLV |
| | | periplasmic | | | KITPQYAGYITQLTVSEGQHVIA |
| | | adaptor | | | GEPLYHINGEHENEQGAGTLAA |
| | | subunit | | | MNLSLRTQYAMLSSQETLELRD |
| | | | | | NQQQQAAIKQRIASLQLQVKSAE |
| | | | | | QRLQMAERQVGLTTSVMGSYK |
| | | | | | KLAGTHYVSDIEYQQKQIEVSTA |
| | | | | | QQNVEDQRQGLLQLRTAIEAAK |
| | | | | | DDLNHLIILGGSRKAELDRQLQEI |
| | | | | | RQQQEELAGQENITLTAPVSGTV |
| | | | | | AAVLVRQGQSVRALEPVMTVVP |
| | | | | | DNASLQIELYAASQNAGFIRPGQ |
| | | | | | RVALRFAAFPYQKFGVQYGTIRE |
| | | | | | ISRTTLTPSDLLSVSPVTWKENEG |
| | | | | | RYRVIVRPENPFILAYGKKEPLRP |
| | | | | | GMTLEGDVNLDTRPLWEWLTEP |
| | | | | | LWSLKGKL |
| 6 | WP_044321592.1 | MULTISPECIES: | Pseudomonas | 313 | MLFRPEALDAQKRSTLGTVLLVS |
| | | HlyD | syringae | | PLSMRLAAAVALLFCISMGLFLT |
| | | family efflux | group | | FATYTKRTAASGVLLPETGLIRIY |
| | | transporter | | | SPQAGVITRLDIVEGQHVTDGAV |
| | | periplasmic | | | LMALSSDTQSGEAGGAQRAISLS |
| | | adaptor | | | IKRRQESLSQEIDETLALHLQELE |
| | | subunit | | | GKKRQVAALETEQQKILAQIDLT |
| | | | | | RQRLALTKGLAERYANLQRQDY |
| | | | | | VSRDQLQEKQDAVLDMRLRGEE |
| | | | | | LNRSLLTVRAESARLRAELVELP |
| | | | | | FNQSKQLADLRRRLAENQDRLIE |
| | | | | | SETKREIFITAPTLGEATAIAVSN |
| | | | | | GSRVDSTRPLLSIVPADAKLHAE |
| | | | | | LYLPSRSVGFVRTGDTVMLRYQ |
| | | | | | AYPYQRFGLQPGTVSSISRTALP |
| | | | | | ADEVMTLGNVSEPMREQGPFYR |
| | | | | | VTVALASQTIDGQGMHERLRSG |
| | | | | | MQLDADIMQERLPLYEWMLEPL |
| | | | | | RGIGKRL |
| 6 | WP_029240900.1 | MULTISPECIES: | Pseudomonas | 314 | MLFRSEALDAQKRSTLGTVLLVS |
| | | HlyD | syringae group | | PLSMRLAAAVALLFCISMGLFLT |
| | | family efflux | genomosp. 2 | | FATYTKRTAASGVLLPETGLIRIY |
| | | transporter | | | SPQAGVITRLDIVEGQHVTDGAV |
| | | periplasmic | | | LMALSSDTQSGEAGGAQRAISLS |
| | | adaptor | | | IKRRQESLAQEIDETLALHLQELE |
| | | subunit | | | GKKRQVAALETEQQKILAQIDLT |
| | | | | | RQRLALTKGLAERYANLQRQDY |
| | | | | | VSRDQLQEKQDAVLDMRLRSEE |
| | | | | | LNRSLLTVRAESTRLRAELVELP |
| | | | | | FNQSKQLADLRRRLAENQDRLIE |
| | | | | | SETKREIFITAPTLGEATAIAVSN |
| | | | | | GSRVDSTRPLLSIVPADAKLHAE |
| | | | | | LYLPSRSVGFVRTGDTVMLRYQ |
| | | | | | AYPYQRFGLQPGTVSSISRTALP |
| | | | | | ADEVMTLGNVSEPMREQGPFYR |
| | | | | | VTVALASQTIDGQGMHERLRSG |
| | | | | | MQLDADIMQERLPLYEWMLEPL |
| | | | | | RGIGKRL |
| 6 | WP_065506284.1 | HlyD family | Serratia | 315 | MTGRPPQLYREGYFTAQATKAE |
| | | efflux | inhibens | | GEVIIPPSLSLTAYAWASGALVL |
| | | transporter | | | AILLFLMLGEYTRKTRLEGIVMP |
| | | periplasmic | | | SSGLIKVVARNAGQVTERLVAE |
| | | adaptor | | | GEKVKAGQPLYRLSGEHENGQG |
| | | subunit | | | VGALASVAASLDQQYRMLEHQ |
| | | | | | REQERATSALRQTGVRQRIAQLD |
| | | | | | DELTSLGSALTLVQQQAAFSHSV |
| | | | | | MARYRKLIAKGYVSEIEFQQKQ |
| | | | | | MELSTMREKVEVQRQAQLRLKR |
| | | | | | ELAAAGSELQTLQQQQQGRDAE |
| | | | | | LERQLQGIDQQKIELGAQRDNTL |
| | | | | | TAPVDGDVAVVLARAGQTVKP |
| | | | | | NDPLLMMVADGAQLQVELFAPS |
| | | | | | KAVGFIKPSQRVGLRFASFPYEK |
| | | | | | FGVQYGSTRTITHASLSAGDALL |
| | | | | | QNPLVWKENEGHYRVIVALDKT |
| | | | | | TITAYGRQEPLRVGMAVSADIEL |
| | | | | | DSRRLYEWLLEPLWSLQGRM |
| 6 | WP_048759060.1 | HlyD family | Serratia | 316 | MSERSPQIYREGYFAAQVNKAE |
| | | efflux | liquefaciens | | GEVIIPASLSLTAYAWASILLVLA |
| | | transporter | | | IGLFLILGEYTRKARLDGVVMPS |
| | | periplasmic | | | SGLIKVVARSAGQVTERLVTEGA |
| | | adaptor | | | TVKAGQALYRLSGEYFNGQGIA |
| | | subunit | | | TLASVSDSLKRQYHMLERQRAQ |
| | | | | | ELATRILQQTGVRQRMTQLQDE |
| | | | | | LASAEAAMVEVKQQAELSHSLM |
| | | | | | ARYRKLIGKGYVSELEFQQQKM |
| | | | | | TLSTAREKVEMQRQAQLRLKRE |
| | | | | | LAAAASELRTLAQQQQGRDAEL |
| | | | | | ERQLQGIRQQEIELVAQRDHTLT |
| | | | | | APVDGHVVVVLARAGQTVKQN |
| | | | | | DPLLMMVADGAELQVELFAPSK |
| | | | | | AVGFIKPHQRVGLRFASFPYEKF |
| | | | | | GVQYGATRAITDASLSANDVMQ |
| | | | | | QNPMTWKENEGHYRVIVALEKT |
| | | | | | TITAYGRQEPLRVGMTVSADVE |
| | | | | | LDRRRLYEWLLEPLWSLQGRI |
| 6 | WP_004944112.1 | HlyD family | Serratia | 317 | MTGRPPQLYREGYFAAQATKAE |
| | | efflux | plymuthica | | GEVIIPPSLSLTAYAWASGALVL |
| | | transporter | | | AILLFLMLGEYTRKARLEGVVM |
| | | periplasmic | | | PSSGLIKVVARNAGQVTERLVAE |
| | | adaptor | | | GAAVKAGQPLYRLSGEHFNGKG |
| | | subunit | | | VGALASVAASLDQQYRMLEHQ |
| | | | | | REQEQVTSALRQTGVRQRIAQL |
| | | | | | DDELTSAGSALVLAQQQETLSHS |
| | | | | | VMARYRKLIAKGYVSEIEFQQK |
| | | | | | QMELSTAREKVESQRQAQLRLK |
| | | | | | REWAAAGSELQTLQQQQQGRD |
| | | | | | AELERQLQSIDQQKIELNAQRET |
| | | | | | TLTAPVDGHIAVVLARAGQTVK |
| | | | | | PNDPLLMMVADGAQLQVELFAP |
| | | | | | SKAVGFIKPSQRVGLRFASFPYE |
| | | | | | KFGVQYGSTRAITHASLSAGDA |
| | | | | | MLQNPLVWKENEGHYRVVVAL |
| | | | | | DKTTITAYGRQEPLRVGMAVSA |
| | | | | | DIELDSRRLYEWLLEPLWSLQGR |
| | | | | | M |
| 6 | WP_086957991.1 | HlyD family | Vibrio casei | 318 | MVARAKHLYRPEYFDAQKSDN |
| | | efflux | | | QGSILIDASLNQHLFVAASMFVL |
| | | transporter | | | MGIIFFVVFAEYTRRGTLNGVVS |
| | | periplasmic | | | PTGGIVKVQANDEGYAEKVFVT |
| | | adaptor | | | EGQKILSGEPLYEIKTERYDEFGQ |
| | | subunit | | | GVKKRIVTAIENQITLLNERRNK |
| | | | | | EIEKSQHQLQVIDYDQVRLSEEA |
| | | | | | SILEVVVDLSEQELKLAQSLVDK |
| | | | | | QQVLVNKQFISKIEFQRQRLELIT |
| | | | | | LKTKVQTQRLSLQRLLREKAKL |
| | | | | | KETKETLQLELDIALQEIDRQLQ |
| | | | | | QIDQSKIEYFYQTDTQVVSPVDG |
| | | | | | VVASIFVKEGHSVNKGQPLLVVI |
| | | | | | PKGEEPAIVELYASSRSIGFLQKG |
| | | | | | QGVRLRFDAFPYEKFGVQQGVIS |
| | | | | | SISKSAVSAEMLPNSPLIQSQLSR |
| | | | | | ANGGMGLYQVRVKLDKPTITVY |
| | | | | | GEEQLFVPGMTLTADIELDTRKV |
| | | | | | YEWLLEPLYTIQGRI |
| 6 | WP_069546416.1 | MULTISPECIES: | Vibrio harveyi | 319 | MRQFYREEYLTIKTKTPEARILIR |
| | | HlyD | group | | SSYSQSAMILVCIIIFLAFFLLGFF |
| | | family efflux | | | GEYTRKASLQGVVNPYGGMVN |
| | | transporter | | | VQSGSTGYIEELLVKEGEVVVAG |
| | | periplasmic | | | QAIYKVNTERHNESGEPIKPLLN |
| | | adaptor | | | QSYNAQTKIIRDRIVQEQKRVGIE |
| | | subunit | | | LNSLNDDIDNLNVNIQNTQRIVS |
| | | | | | LSRQELELRQALFADKKKLLKL |
| | | | | | GHISEVEYANEQLKVNEVASRY |
| | | | | | ESNLLNYNAKNRELAALKHAVK |
| | | | | | STEINSAITIKDLDRQLEVVEQER |
| | | | | | SEFLYQTDTRVVAPITGVVASVL |
| | | | | | VKEGHSVIAGQTLLVIVPEGEVF |
| | | | | | VEVYAPSRDIGFINLGQKVKLRF |
| | | | | | EAFPYQKFGVQGGIVESVSRTAV |
| | | | | | SPDVITNQRLIKADKVEGLYQVR |
| | | | | | ITLNKQTITAYGREELLKSGMAV |
| | | | | | TADVEVDTRKLYEWVLEPMYSI |
| | | | | | KGRLE |
| 7 | VC1_MFP_cvaA | Colicin V | Vibrio | 320 | MNLFREEVFQAKKNRLFGDVVI |
| | | secretion | cholerae | | NQPAFLYVIITAVFVLFIVLSIYIS |
| | | protein | | | QSSYSRKETVKGYLIPEKGLMKV |
| | | | | | FLNRSGVVGDLLVEEGSKVNAG |
| | | | | | DGLAKIINSQSLSSGVELSDILVD |
| | | | | | ELEKQKVTLSEELRVIYKIQENDI |
| | | | | | NQISLKLAQLEDSLNVIKKIKLTV |
| | | | | | QNKLELKERLFTNNKELYERGYI |
| | | | | | SKINLFDSHEEYLNIRESMDGLD |
| | | | | | REILDLVAKIADFEVKKNSIPNEV |
| | | | | | ELKRISIERRISEINMQILELKNKY |
| | | | | | EFIVKAPESGTVTAIQAYTGTNIN |
| | | | | | NNVPLLSIIPEDSILEIEMLLPTRS |
| | | | | | AGFIQIGDPVRIRFDAFPYQKFGS |
| | | | | | IKGEVTKIDKALLLPTDQMLPIRL |
| | | | | | EEASYKVSAKLEKQEIVAYDKSF |
| | | | | | SLKVGMIAEADIILENRSLLDWL |
| | | | | | LDPIYALQGKL |
| 7 | WP_036275542.1 | MULTISPECIES: | Methylomonas | 321 | MKHELFRQQALDFKKDKPLGEV |
| | | HlyD | | | IHVESLSFSFLTALAVSSALVLM |
| | | family efflux | | | AFAFWGEYTRKSHVNGYLSPSM |
| | | transporter | | | GLIKVYAPQAGTLIEKHIREGQA |
| | | periplasmic | | | VKAGDTLFVLSTESSSRETPQAQ |
| | | adaptor | | | AAAIEQLKQRRESLETELVKQEQ |
| | | subunit | | | IDQIQYRSVLDRLHGMQRELQQ |
| | | | | | VNSEIDTQQQRLAGAAATSQRY |
| | | | | | QQLLTTKFVSAVQAQQKQDEWL |
| | | | | | DHQAKLQALQRVQMTLQRDIRS |
| | | | | | AQLEADSSQMKFKNQRAAIERNI |
| | | | | | STLAQNITESESRRNIVITAPHDG |
| | | | | | TVTTILAEQGQNATTANPLLSILP |
| | | | | | LGAKLQAQLLVPSSAVGFVEQG |
| | | | | | QTVSVRYQAFPYQRFGSHQGRIV |
| | | | | | EIAKTLITPKEADLPVTLQEPVYR |
| | | | | | VTVEIDKQAVQAYRQDIPLQSG |
| | | | | | MLLDADIWLDHRRLIEWVFDPL |
| | | | | | YSILGRV |
| 7 | WP_011074383.1 | HlyD family | Shewanella | 322 | MSDLFRQQVVNEQKQRLYGDIS |
| | | efflux | oneidensis | | LAQPLSIYTISIAILLIVTAIILFLYF |
| | | transporter | | | SHYARKETVRGYLVPDKGVIKT |
| | | periplasmic | | | YANRIGNVDILHVKEGSDVNAG |
| | | adaptor | | | DPLVTVIIRSSMASGFELSETLISE |
| | | subunit | | | LKQQQSILNQELDNNIELNAAET |
| | | | | | LRLKKRLSDLSESMKVLSRQKQ |
| | | | | | LLADKLNIQVAQKKQHDKLYKD |
| | | | | | GYLSELDYQLQLSKLIEVKQEVE |
| | | | | | NLESNKISIDRELNQTHAELVSLP |
| | | | | | FQFNLKQSDVHKRQSDIQRQLNE |
| | | | | | AENSYTFVIRAQESGTVAAVSVV |
| | | | | | EGEFIASNRPLMSIIPKGSSLVAE |
| | | | | | LLLPTRSAGFVKQGDEARLRFDA |
| | | | | | FPYQRFGFLHSEVLRVDKALLLD |
| | | | | | GEADLPVKLSEPVYRIKTTLSAQ |
| | | | | | DMQAYGEAFPLKSGMLLEADIV |
| | | | | | LDRRTLLDWLLDPIYSLRGRVS |
| 7 | WP_014611436.1 | HlyD family | Shewanella | 323 | MATLFRQQVVDEQKQRLYGEIS |
| | | efflux | putrefaciens | | LAQPLSIYTVSVSIFFTVLVVALF |
| | | transporter | | | LYFSHYARKETVRGYLVPDKGV |
| | | periplasmic | | | IKTYANRNGNVEVLHVKEGANV |
| | | adaptor | | | NAGDPLVTVIIRSSMTSGIELSEV |
| | | subunit | | | LINELKHQQQILNEEIKNNSALNL |
| | | | | | SETKRLEKRLSDLIESMAVVSRQ |
| | | | | | KALLDDKLTIQINQNKQHDKLY |
| | | | | | KDGYLSELDYQFQLSKLIEVKQE |
| | | | | | LENVESNRVSINSELNQTRSELAS |
| | | | | | LPFQFNLKQSDVHKRQSDIQKQL |
| | | | | | NEAENSYRFVVRAQESGTVASV |
| | | | | | SVVEGEFIANNRPLMSIIPKGSSL |
| | | | | | VAELLLPTRSAGFVKQGDEARL |
| | | | | | RFDAFPYQRFGFLHSEVLRVDKA |
| | | | | | LLLDGEADLPVKLTEPVYRIRTT |
| | | | | | LSAQDMQAYGEAFPLRSGMLLE |
| | | | | | ADIVLDKRTLLDWLLDPIYSLRG |
| | | | | | RVS |
| 7 | WP_074052066.1 | HlyD family | Vibrio harveyi | 324 | MTQPFSFYFKVAFVFMSFFLIFIY |
| | | efflux | | | LNQYSYTRKESVKGYLTPKKGL |
| | | transporter | | | VKVYLNREGIIEELMVSEGKEVK |
| | | periplasmic | | | IGDGLAKIKNSQSLSSGEELSESIS |
| | | adaptor | | | NELKKQKIVLTNEISILKEIKYKD |
| | | subunit | | | NLKLENQLIKLYKSYSSAKKLRE |
| | | | | | TSLKKLKIKKDNYLINRDLKDKG |
| | | | | | FLSRNEFLVSLEEYLNAQENVDR |
| | | | | | LDQNLVSIELEISIKESEKNSLPEN |
| | | | | | LQLKLTTIERQISSINTQLYELNN |
| | | | | | QYEFIKKAPESGIVTAIQSYVGSN |
| | | | | | VNLKTPLLSIIPKDSPLELEMLLP |
| | | | | | TRSSGFVQVGDHVNIRFDAFPYQ |
| | | | | | KFGMVKGTIISVDKALLLPSDKA |
| | | | | | LPFAVNEAMYQVRAELDNQTVL |
| | | | | | AYGKSYPLKVGMIADADIILEKR |
| | | | | | SLLDWLLEPLYSVKGRF |
| 8 | L_MFP_mclA | HlyD family | Escherichia | 325 | MKWQGRAILLPGIPLWLIMLGSI |
| | | secretion | coli | | VFITAFLMFIIVGTYSRRVNVSGE |
| | | protein | | | VTTWPRAVNIYSGVQGFVVRQF |
| | | | | | VHEGQLIKKGDPVYLIDVSKSTR |
| | | | | | SGIVTDNHRRDIENQLVRVDNIIS |
| | | | | | RLEESKKITLDTLEKQRLQYTDA |
| | | | | | FRRSSDIIQRAEEGIKIMKNNMEN |
| | | | | | YRNYQAKGLINKDQLTNQVALY |
| | | | | | YQQQNNLLSLSGQNEQNALQITT |
| | | | | | LESQIQTQAADFDNRIYQMELQR |
| | | | | | YELQKELVNTDVEGEIIIRALTDG |
| | | | | | KVDSLSVTVGQMVNTGDSLLQV |
| | | | | | IPENIENYYLILWVPNDAVPYISA |
| | | | | | GDKVNIRYEAFPAEKFGQFSATV |
| | | | | | KTISRTPASTQEMLTYKGAPQNT |
| | | | | | PGASVPWYKVIAMPEKQIIRYDE |
| | | | | | KYLPLENGMKAESTLFLEKRRIY |
| | | | | | QWMLSPFYDMKHSATGPLND |
| 9 | V_MFP_cvaA | Colicin V | Escherichia | 326 | MKWQGRAILLPGIPLWLIMLGSI |
| | | secretion | coli | | VFITAFLMFIIVGTYSRRVNVSGE |
| | | protein CvaA | | | VTTWPRAVNIYSGVQGFVVRQF |
| | | | | | VHEGQLIKKGDPVYLIDISKSTR |
| | | | | | NGIVTDNHRRDIENQLVRVDNIIS |
| | | | | | RLEESKKITLDTLEKQRLQYTDA |
| | | | | | FRRSSDIIQRAEEGIKIMKNNMEN |
| | | | | | YRYYQSKGLINKDQLTNQVALY |
| | | | | | YQQQNNLLSLSGQNEQNALQITT |
| | | | | | LESQIQTQAADFDNRIYQMELQR |
| | | | | | LELQKELVNTDVEGEIIIRALSDG |
| | | | | | KVDSLSVTVGQMVNTGDSLLQV |
| | | | | | IPENIENYYLILWVPNDAVPYISA |
| | | | | | GDKVNIRYEAFPSEKFGQFSATV |
| | | | | | KTISRTPASTQEMLTYKGAPQNT |
| | | | | | PGASVPWYKVIATPEKQIIRYDE |
| | | | | | KYLPLENGMKAESTLFLEKRRIY |
| | | | | | QWMLSPFYDMKHSATGPIND |
| 10 | ER14_MFP_cvaA | Membrane | Pantoea | 327 | MFRKEFIHSRSRKWKAKVILLPG |
| | | fusion protein | agglomerans | | ISMWIYCFFSTVFLVILISFIVAGS |
| | | | | | YTRRISVSGEYMTWPQPVYIYSD |
| | | | | | VQGTVAEQKVKPGQSVKKNEIL |
| | | | | | YTINVSRSTNGGVVSENRKAAIK |
| | | | | | GQIHRLEETITLLKSSKVVTLNTL |
| | | | | | ASEEKQYQDALLKSSDIVNLAQ |
| | | | | | DGILKLKKNVENYHDYQQRGLV |
| | | | | | NKDQVANQTAIYYQQQNNLLNL |
| | | | | | ITQNQQNALQITTIESQILTQRSE |
| | | | | | YDSKIYQAEMQLFDLKKEIINEN |
| | | | | | ARTSVSVPSLTEGTVQSMNVSPG |
| | | | | | QNITQGESLSLIKPRHVESSQIIVF |
| | | | | | APNNALPYLKTGSPVRISFAAFP |
| | | | | | YENFGFFSGKISSVSRVAAPSAE |
| | | | | | MKKYHFSGEDIKNSGSYYKITIA |
| | | | | | PEKSIIRYANSDIPLESGMEAKVT |
| | | | | | LYLEKRRLYQWVMGPLYALKN |
| | | | | | SLNEGISEEKNQIFY |
| 11 | ER31_MFP_cvaA | Membrane | Pantoea allii | 328 | MLYRKEVTEARRNYWSGKAVLI |
| | | fusion protein | | | SDQHGRYVMAFTLFFFTAFFILIT |
| | | | | | SCSYTRRISVSGEVVSTPRAVTVF |
| | | | | | SPQQGFIISRAVKQGDHVDKGQP |
| | | | | | LYRIDVSRTTTSGVVSQRHHESI |
| | | | | | QKRITTLNQIAADISSNRALTLSM |
| | | | | | LAEEKSRYESALLRSSEIVTHARE |
| | | | | | GLKLMKENMDNYRQYQHQGLI |
| | | | | | NRDQLINQTALYYQQQNDMLGI |
| | | | | | ESQNEQNALQVMNLRSALQTQA |
| | | | | | ANFDNQLNQIAIQKSALESDLTD |
| | | | | | ADAGSDQIITSPVDGTVDTLSVT |
| | | | | | PGQMVTSGDSLLQIIPGDAHYRT |
| | | | | | LVLWVPDSAIPYLSAGEPVNIRY |
| | | | | | DAFPAEKFGQFPGKIASVAHSPA |
| | | | | | SWQEMSTYPGAPLHSLTGPQTW |
| | | | | | YKILVNPASDHFTYKNKTIRAEN |
| | | | | | GMKASVTLFLEERKLYQWILSPL |
| | | | | | YDIRDSATGAIHE |

In some embodiments, the membrane fusion protein comprises an amino acid sequence that has about 70% or greater (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity to any of the amino acid sequences described herein (e.g., the amino acid sequence of E coli CvaA). For example, the membrane fusion protein may contain an amino acid sequence that has about 70% or greater (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity to SEQ ID NOS: 191, 196, 200, 243, 250, 263, 293, 301, 304, 307, 308, 314, 320, 321, 325, 326, 327, or 328. Additional amino acid residues may be present at the N-terminus or C-terminus of any of these sequences (e.g., a starting methionine (“M”) residue at the N-terminus, or a sequence containing a purification tag at the C-terminus).

### D. Outer Membrane Channel Proteins

Gram-negative bacterial cells according to the present disclosure also contain nucleic acids encoding outer membrane channel proteins, such as Escherichia coli K12 TolC (UniProt Accession #P02930). In some embodiments, the bacterial cell is transformed with a fourth nucleic acid encoding the outer membrane channel protein. Alternatively, the outer membrane channel protein may be naturally expressed by the cell used for peptide secretion, such that transformation with an exogenous nucleic acid encoding the outer membrane channel protein is not necessary.

In some embodiments, the outer membrane channel protein comprises an amino acid sequence as set forth in Table 8.

TABLE 8

| | ID/RefSeq | | | SEQ | |
|---|---|---|---|---|---|
| Group | Accession | Description | Taxon | ID NO | Sequence |
| 1 | tr\|Q1R6U8\| | Outer | Escherichia coli | 329 | MIALNTASPQGMQMKKLLPILI |
| | Q1R6U8_ECOUT | membrane | | | GLSLSGFSSLSQAENLMQVYQQ |
| | | channel | | | ARLSNPELRKSAADRDAAFEKI |
| | | protein | | | NEARSPLLPQLGLGADYTYSNG |
| | | | | | YRDANGINSNATSASLQLTQSIF |
| | | | | | DMSKWRALTLQEKAAGIQDVT |
| | | | | | YQTDQQTLILNTATAYFNVLNA |
| | | | | | IDVLSYTQAQKEAIYRQLDQTT |
| | | | | | QRFNVGLVAITDVQNARAQYD |
| | | | | | TVLANEVTARNNLDNAVEQLR |
| | | | | | QITGNYYPELAALNVENFKTDK |
| | | | | | PQPVNTLLKEAEKRNLSLLQAR |
| | | | | | LSQDLAREQIRQAQDGHLPTLD |
| | | | | | LTASTGISDTSYSGSKTRGAAGT |
| | | | | | QYDDSNMGQNKVGLSFSLPIYQ |
| | | | | | GGMVNSQVKQAQYNFVGASE |
| | | | | | QLESAHRSVVQTVRSSFNNINA |
| | | | | | SISSINAYKQAVVSAQSSLDAM |
| | | | | | EAGYSVGTRTIVDVLDATTTLY |
| | | | | | NAKQELANARYNYLINQLNIKS |
| | | | | | ALGTLNEQDLLALNNALSKPVS |
| | | | | | TNPENVAPQTPEQNAIADGYAP |
| | | | | | DSPAPVVQQTSARTTTSNGHNP |
| | | | | | FRN |
| 1 | WP_021231688.1 | MULTISPECIES: | Aeromonas | 330 | MKRTLLSVMVLLGVSAGAHAE |
| | | outer | | | NLLDIYQQAQIKDPQLLQSKAV |
| | | membrane | | | RDQAFEKINESRAPLLPQIDLGA |
| | | channel | | | GLNYLQNKNDTQSNTNAKGEV |
| | | protein TolC | | | TLSQSLYRRSNWVNLDISEKQA |
| | | | | | TQSDVAYNLEIQNLMVRTAKA |
| | | | | | YFDVLKAMDTLEFVRANKVAV |
| | | | | | ERQLEQTQQRFEVGLTAITDVH |
| | | | | | EAEASRDQALADEINAENSLDN |
| | | | | | SYETLRELTGIDHRNLDVLNTD |
| | | | | | RFSPQKTAFGSDKWLELALDKN |
| | | | | | LQLHSARISKDIAKEQIDLAKTG |
| | | | | | HEPTLDLGAGLQTSNTDYKLDS |
| | | | | | MPGGNDNQANIGLTFKLPIYSG |
| | | | | | GATSSKVKQAQYNYVAASELL |
| | | | | | EKNFRSVQSTVRSSYNNVNASI |
| | | | | | GSVRAYSQSVVSADSALKATEA |
| | | | | | GYEVGTRTIVDVLDSTRKLYEA |
| | | | | | KQKLSEARYNYILNILSLKQAA |
| | | | | | GILEAQDLVEVNQGLMPATAK |
| | | | | | AKG |
| 1 | WP_029313618.1 | MULTISPECIES: | Aeromonas | 331 | MKRTLLSVMVMLGVSGAAHA |
| | | outer | | | ENLLDIYQQAQIKDTQLLESKA |
| | | membrane | | | KRDQAFEKINESRAALLPQINLG |
| | | channel | | | AGLNYLQNKNDTMTNTGATGS |
| | | protein TolC | | | VTLDQSIYRRSNWVNLDLTEKS |
| | | | | | ATQSDVAYNLEYQNLMLRTAQ |
| | | | | | AYFDVLKAMDTLEFVRANKTA |
| | | | | | VERQLEQTQQRFEVGLTAITDV |
| | | | | | HEAEAERDQALADEINAENTLD |
| | | | | | NSYESLRELTGIDHRQLDVLNT |
| | | | | | DRFSPQKTAFNSDKWLELALDK |
| | | | | | NLELHSARIGKDIAKEQIDLAKT |
| | | | | | GHEPTLDLGAGLNAGYNDYKD |
| | | | | | EIRNPESNSNQGNIGLNFKLPIY |
| | | | | | TGGATTSQVKQAQFNYVAASE |
| | | | | | QLERSFRSVQSTVRSSYNNVNA |
| | | | | | SIGSVRAYSQSVISADSALKATE |
| | | | | | AGYEVGTRTIVDVLDSTRKLYQ |
| | | | | | AKQKLSEARYNYILSILSLKQA |
| | | | | | AGILEQKDLEEVNQGLMPAAQ |
| | | | | | VTKKS |
| 1 | WP_045523101.1 | outer | Aeromonas caviae | 332 | MKRTLLSVMVMLGVSGAAHA |
| | | membrane | | | ENLLDIYQQAQIKDTQLLESKA |
| | | channel | | | KRDQAFEKINESRAALLPQINLG |
| | | protein TolC | | | AGLNYLQNKNDTMTNTGATGS |
| | | | | | VTLDQSIYRRSNWVNLDLTEKS |
| | | | | | ATQSDVTYNLEYQNLMLRTAQ |
| | | | | | AYFDVLKAMDTLEFVRANKTA |
| | | | | | VERQLEQTQQRFEVGLTAITDV |
| | | | | | HEAEAERDQALADEINAENTLD |
| | | | | | NSYESLRELTGIDHRQLDVLNT |
| | | | | | DRFSPQKTAFNSDKWLELALDK |
| | | | | | NLELHSARIGKDIAKEQIDLAKT |
| | | | | | GHEPTLDLGAGLNAGYNDYKD |
| | | | | | EIRNPESNSNQGNIGLNFKLPIY |
| | | | | | TGGATTSQVKQAQFNYVAASE |
| | | | | | QLERSFRSVQSTVRSSYNNVNA |
| | | | | | SIGSVRAYSQSVISADSALKATE |
| | | | | | AGYEVGTRTIVDVLDSTRKLYQ |
| | | | | | AKQKLSEARYNYILSILSLKQA |
| | | | | | AGILEQKDLEEVNQGLMPAAQ |
| | | | | | VTKKS |
| 1 | WP_124813304.1 | outer | Aeromonas caviae | 333 | MFLPILALLSSYTHAEGLLDIYN |
| | | membrane | | | LAKQKDPQLLESKAMRDQSFE |
| | | channel | | | KINEEQASFLPKINLGLSANYVD |
| | | protein TolC | | | DNLDMQTGSATNSAINLEQSLY |
| | | | | | NSANWINLDLAAKNATLSDVS |
| | | | | | YVLEHQNLVLRTIQTYFNVLQA |
| | | | | | EDLLIYSQATKKAIQRQLAQTQ |
| | | | | | QRFEVGLAAITDVHEAEAAFDQ |
| | | | | | SFADEIYAENTLENSYEALRELT |
| | | | | | GLEHRKLNKLDTERFSPQKTLW |
| | | | | | KIDKWTDAAIEKNLALHQARIK |
| | | | | | KELAKMQIDLSKSGHKPLVDLT |
| | | | | | MGLSSTYNNYKDNTVNTDGYS |
| | | | | | NQGDIGIKFSLPLYTGGATASQ |
| | | | | | VKQSSFDYVAASEQLEKSYRSV |
| | | | | | MSAVKSSYNNVNASIGAVRAY |
| | | | | | QQAVVSAESALKATEAGYEVG |
| | | | | | TRTIIDVLDANRKLYDAKTKLAI |
| | | | | | SRYSYIVNVATLKLNSGSLGKK |
| | | | | | EIIDIDNGLVNLR |
| 1 | WP_198497256.1 | outer | Aeromonas caviae | 334 | MKRTLLSVMVMLGVSGAAHA |
| | | membrane | | | ENLLDIYQQAQIKDTQLLESKA |
| | | channel | | | KRDQAFEKINESRAALLPQINLG |
| | | protein TolC | | | AGLNYLQNKNDTMTNTGATGS |
| | | | | | VTLDQSIYRRSNWVNLDLTEKS |
| | | | | | ATQSDVAYNLEYQNLMLRTAQ |
| | | | | | AYFDVLKAMDTLEFVRANKTA |
| | | | | | VERQLEQTQQRFEVGLTAITDV |
| | | | | | HEAEAERDQALADEINAENTLD |
| | | | | | NNYESLRELTGIDHRQLDVLNT |
| | | | | | DRFSPQKTAFNSDKWLELALDK |
| | | | | | NLELHSARIGKDIAKEQIDLAKT |
| | | | | | GHEPTLDLGAGLNAGYNDYKD |
| | | | | | EIRNPESNSNQGNIGLNFKLPIY |
| | | | | | TGGATTSQVKQAQFNYVAASE |
| | | | | | QLERSFRSVQSTVRSSYNNVNA |
| | | | | | SIGSVRAYSQSVISADSALKATE |
| | | | | | AGYEVGTRTIVDVLDSTRKLYQ |
| | | | | | AKQKLSEARYNYILSILSLKQA |
| | | | | | AGILEQKDLEEVNQGLMPAAQ |
| | | | | | VTKKS |
| 1 | WP_025201111.1 | outer | Aeromonas | 335 | MKRTLLSAMVLLGVSAGTHAE |
| | | membrane | hydrophila | | NLLDIYQQAQIKDTQLLESKAK |
| | | channel | | | RDQAFEKINESRAALLPQINLGA |
| | | protein TolC | | | GLNYLQNKGDTQTNSNATGSL |
| | | | | | SLDQSIYRRSNWVNLSLTEKSA |
| | | | | | TQSDVAYNLEIQNLMLRTAQA |
| | | | | | YFNVLKAMDTLEFVRANKAAV |
| | | | | | ERQLEQTQQRFEVGLTAITDVH |
| | | | | | EAEAERDQALADEINAENTLDN |
| | | | | | SYESLRELTGIDHRQLDVLNTD |
| | | | | | RFSPQKTPFNSDKWLELALDKN |
| | | | | | LQLHSARIGKDIAKEQIDLAKTG |
| | | | | | HEPTLDLGAGLSSTYSDYKDEI |
| | | | | | RNPESNANQANIGLNFKLPLYT |
| | | | | | GGATTSQVKQSQFNYVAASEQ |
| | | | | | LERSFRNVQSTVRSSYNNVNAS |
| | | | | | IGSVRAYGQSVISADSALKATE |
| | | | | | AGYEVGTRTIVDVLDSTRKLYQ |
| | | | | | AKQKLSEARYNYILSILSLKQA |
| | | | | | AGTLEQKDLEDVNQGLMPASQ |
| | | | | | AKSKS |
| 1 | WP_011898310.1 | outer | Aeromonas | 336 | MKRTLLSAMVLLGVSAGAHAE |
| | | membrane | salmonicida | | NLLDIYQQAQIKDTQLQESKAK |
| | | channel | | | RDQAFEKINESRAALLPQINLGA |
| | | protein TolC | | | GLNYLQNKGDTQTNSNATGSL |
| | | | | | SLDQSIYRRSNWVNLDLTEKSA |
| | | | | | TQSDVAYNLEIQNLMLRTAQA |
| | | | | | YFNVLKAMDTLEFVRANKAAV |
| | | | | | ERQLEQTQQRFEVGLTAITDVH |
| | | | | | EAEAERDQALADEINAENTLDN |
| | | | | | SYESLRELTGIDHRQLDVLNTE |
| | | | | | RFSPQKTPFNSDKWLELALDKN |
| | | | | | LQLHSARIGKDIAKEQIDLAKTG |
| | | | | | HEPTLDLGAGLSSTYSDYKDEI |
| | | | | | RNPESNSNQGNIGLNFKLPLYT |
| | | | | | GGATTSQVKQSQFNYVAASEQ |
| | | | | | LERSFRSVQSTVRSSYNNVNASI |
| | | | | | GSVRAYGQSVISADSALKATEA |
| | | | | | GYEVGTRTIVDVLDSTRKLYQA |
| | | | | | KQKLSEARYNYILSILSLKQAA |
| | | | | | GTLEQKDLEEVNQGLIPAAQVK |
| | | | | | NKS |
| 1 | WP_005335902.1 | outer | Aeromonas | 337 | MKRTLLSVMVLLGVSAGAHAE |
| | | membrane | veronii | | NLLDIYQQAQIKDPQLLQSKAV |
| | | channel | | | RDQAFEKINESRAPLLPQIDLGA |
| | | protein TolC | | | GLNYLQNKNDTQSNTNAKGEV |
| | | | | | TLSQSLYRRSNWVNLDISEKQA |
| | | | | | TQSDVAYNLEIQNLMVRTAKA |
| | | | | | YFDVLKAMDTLEFVRANKVAV |
| | | | | | ERQLEQTQQRFEVGLTAITDVH |
| | | | | | EAEASRDQALADEINAENSLDN |
| | | | | | SYETLRELTGIDHRNLDVLNTD |
| | | | | | RFSPQKTAFGSDKWLELALDKN |
| | | | | | LQLHSARISKDIAKEQIDLAKTG |
| | | | | | HEPTLDLGAGLQTSNTDYKLDS |
| | | | | | IPGGNDNQANIGLTFKLPIYSGG |
| | | | | | ATSSKVKQAQYNYVAASELLE |
| | | | | | KNFRSVQSTVRSSYNNVNASIG |
| | | | | | SVRAYSQSVVSADSALKATEAG |
| | | | | | YEVGTRTIVDVLDSTRKLYEAK |
| | | | | | QKLSEARYNYILNILSLKQAAGI |
| | | | | | LEAQDLVEVNQGLMPATAKAK |
| | | | | | G |
| 1 | WP_008739333.1 | TolC family | Alcanivorax | 338 | MRTKRITYAAAALLLAATGVA |
| | | outer | pacificus | | QAATLADVLESAWQADSQWSG |
| | | membrane | | | ALRTWEAEQENVVQGRAGLLP |
| | | protein | | | SVNASYSWLDNDVEVTSSPGGE |
| | | | | | TDFETQTLTLSLVQPLFRPGAW |
| | | | | | YAYKQADAATSVAAANFQQAR |
| | | | | | QDFLQRVATQYFGVLQSWENL |
| | | | | | VSIRAEERAIGRQLEQTRERFDV |
| | | | | | GLVAITDVHEAEAAFDLTKVER |
| | | | | | ILAEADFDIARDRLEALTGRTW |
| | | | | | ESLAALQEELPLTGPEPANPQQ |
| | | | | | WVELARQQNPTILAALYQSESS |
| | | | | | RHFASQQAWAHGPTVDLVAQH |
| | | | | | QRYKNDASGPTSTLNLPDTRTN |
| | | | | | AYGIEVNLPLFQGGGVNSRRKQ |
| | | | | | AALQHEASQDLYQQTWRDVG |
| | | | | | QQTLGTYRVVSANALRIRARAQ |
| | | | | | AIRSADSALEATQAGYEVGTRN |
| | | | | | IVDVLNAQRTLFAARRDYANA |
| | | | | | RYEYILDSLGLKALAGVLTEDD |
| | | | | | LVQVNNWLAPADLVELDLVSA |
| | | | | | GTDDALDE |
| 1 | WP_014948205.1 | outer | Alteromonas | 339 | MKRTLLSLVIGVSMTTGALADD |
| | | membrane | macleodii | | LMQVYQQALANDPLVNQAKA |
| | | channel | | | QRDAAFEGISLSRASLLPQISGS |
| | | protein TolC | | | VGYTHAESERFQQLGPDTSFTS |
| | | | | | DTTVDTTDYGISLTMSLYDHAN |
| | | | | | WLNLNRAEKIAEQSDAQLAAS |
| | | | | | MQDVIVRTVTAYFDVLRARDN |
| | | | | | VEFVGAEKRAIERQLEQTRQRF |
| | | | | | EVGLTAITDVHEAQANYDSTVA |
| | | | | | QEIQAKNQLEFALEALRVITGK |
| | | | | | YHDRLFGLNTENFSATMPVPET |
| | | | | | VDSWLETAQDQNLALLVDRLA |
| | | | | | MDVAKEDIAIARSGHLPTLALS |
| | | | | | GRYGRSKDDVEGEFITDDGQTA |
| | | | | | PVAYDTPYLDSKSIGVTLSVPIY |
| | | | | | QGGSVSAQTSQAKYNYVAASQ |
| | | | | | AAEQTYRQTVQSVRSSFNNVK |
| | | | | | ASISTIRALEQSVVSADSALKAT |
| | | | | | EAGFDVGTRTIVDVLDSTRNLF |
| | | | | | DARRNLAGARYDFIQSVVTLKQ |
| | | | | | AAGTLTGEDVAMINRGLKPAET |
| | | | | | KTNG |
| 1 | WP_075608306.1 | outer | Alteromonas | 340 | MKRTLLSLIIGLGITSSALADDL |
| | | membrane | pelagimontana | | LQVYQQALSSDPVVNRAQAER |
| | | channel | | | DAAYSGIPISRANLLPQIAGTVG |
| | | protein TolC | | | YTFASRESTQFAGSDEQNLSLIT |
| | | | | | LDSETESIDYGIDMSMSLYDHA |
| | | | | | NWVGLNRAEKVAEQSDAQLA |
| | | | | | AAMQNLIVRTVTAYFDVLRAK |
| | | | | | DNLEFVRAEKRAIERQLEQTKQ |
| | | | | | RFEVGLTAITDVHEAQANYDTT |
| | | | | | VAQEIQAENQVEVALEGLRVIS |
| | | | | | GKYHDKLFTLDTDSFSASLPTP |
| | | | | | AKVEDWLSIAEDSNLSLLVNRL |
| | | | | | ALDIAKEDIAAAQAGHYPSLGL |
| | | | | | SASAGRTKNDISSDNINYTTPYL |
| | | | | | NNYSVGVNLSVPIFQGLRVSSQ |
| | | | | | TEQARYQYIAVSQDVELTYRQT |
| | | | | | VQSIRSSFNDLKAAISTIRALEQ |
| | | | | | AVVSAKSALQATEAGFDVGTR |
| | | | | | TIVDVLDSTRNLFDARRNLASA |
| | | | | | RYDFIQAVVDLKQAAGSLTSED |
| | | | | | IVTINRGLEPQESKQPQ |
| 1 | WP_062152497.1 | TolC family | Beggiatoa | 341 | MMYKRLSYLLLTSCLVVPAIQA |
| | | outer | leptomitoformis | | ETLLDLYALAKENDAQLKVSGS |
| | | membrane | | | DRLIALEAKPQAQASLLPQVVL |
| | | protein | | | GADATESWNTENWMSGSDTER |
| | | | | | TALGYSVSLSYPLYRRDRQILLE |
| | | | | | QADIGVKIAEESFASQQQALIQR |
| | | | | | LSSAYFTILNAEDNLRFTRSARE |
| | | | | | AFRRQLDQTQQRFDVGLIAITD |
| | | | | | VKEAQSGFDQAVSDEITAQNTV |
| | | | | | DQSYESLREITGSYHKLLATLK |
| | | | | | ADTPLLPPDPQDIDAWTKTALE |
| | | | | | QNPQVIVQLATIENARQEVERQ |
| | | | | | RAGNLPTVDLTAQHAYNDTVR |
| | | | | | GDESNGMRTNNSIGVSLSYSLY |
| | | | | | EGGAIRSKIREAQQRYVQELDR |
| | | | | | LEQVRRSIQNQTHTNYLNVLSG |
| | | | | | ISRVKASRQAVESALTALEAIET |
| | | | | | GFDVGTRTSVDVLNARRDLLD |
| | | | | | AQRNYSQSRYQYVLSTVTLKQS |
| | | | | | AGLLRLEDLVAINNWLTTPSQV |
| | | | | | AEPATTEKKTEKETTKKSSAKK |
| 1 | WP_024911273.1 | outer | Chania | 342 | MKKLLPLLIGLSLGGFSAMSQA |
| | | membrane | multitudinisentens | | ENLLQVYKQAKESNPDLRKTA |
| | | channel | | | ADRDAAFEKISEARSPLLPQLGL |
| | | protein TolC | | | SAGYNHTNGFRDANGVNSNTT |
| | | | | | NGSLSLTQTIFDMSAWRALTQQ |
| | | | | | EKAAGISDVTFQTASQKLILDTA |
| | | | | | TAYFNVLLAIDSLSYVQANKQA |
| | | | | | VYRTLDQTTQRFNVGLVAITDV |
| | | | | | QNARSNYDSVLASEVTARNNL |
| | | | | | DNSVEVLRQLTGTFYPELASLN |
| | | | | | TDKFSTQRPDAVNNLLKEAEKR |
| | | | | | NLSLLSARLNQDLAREQIKSAE |
| | | | | | TGYMPTISASASTGLTNTRYNG |
| | | | | | SNSDSRTNSDNGQNQVGVTLSV |
| | | | | | PIYNGGVTNSRVQQAQYNFVG |
| | | | | | ASEQLESTHRSVVQTVRSSFNN |
| | | | | | VSASISSISAYAQAVVSAQSSLD |
| | | | | | ATEAGYEVGTRTIVDVLSATTT |
| | | | | | LYNAKQQLASARYSYLTNQLTI |
| | | | | | KSALGTLNENDLMLLNGVLGK |
| | | | | | TVPTSPDMVESATPQPARSN |
| 1 | WP_003828407.1 | MULTISPECI | Citrobacter | 343 | MKKLLPILIGLSLSGFSTLSQAE |
| | | outer | | | NLMQVYQQARLSNPELRKSAA |
| | | membrane | | | DRDAAFEKINEARSPLLPQLGL |
| | | channel | | | GADYTYSNGYRDANGVNSNAT |
| | | protein TolC | | | SASLQLTQSIFDMSKWRALTLQ |
| | | | | | EKSAGIQDVTYQTDQQTLILNT |
| | | | | | ATAYFNVLNAIDVLSYTQAQK |
| | | | | | DAVYRQLDQTTQRENVGLVAIT |
| | | | | | DVQNARSQYDTVLANEVTARN |
| | | | | | NLDNAVEQLRQVIGNYYPELA |
| | | | | | SLNVDGFKTNKPQAVNALLKE |
| | | | | | AENRNLSLLQARLSQDLAREQI |
| | | | | | RLAQDGHLPTLDLTASTGVSDT |
| | | | | | SYSGSKTHGGATGNQYDDSNM |
| | | | | | GQNKIGLSFSLPLYQGGMVNSQ |
| | | | | | VKQAQYNFVGASEQLESAHRS |
| | | | | | VVQTVRSSFNNINASISSINAYK |
| | | | | | QAVVSAQSSLDAMEAGYSVGT |
| | | | | | RTIVDVLDATTTLYNAKQQLAN |
| | | | | | ARYTYLINQLNVKSALGTLNEQ |
| | | | | | DLVALNNTLGKPVSTTPDTIAPE |
| | | | | | NAQQDAAADGYTSNSATPAAQ |
| | | | | | PTAARSTSSNGNNPFRN |
| 1 | WP_042998171.1 | MULTISPECIES: | Citrobacter | 344 | MKKLLPILIGLSLSGFSTLSQAE |
| | | outer | | | NLMQVYQQARLSNPELRKSAA |
| | | membrane | | | DRDAAFEKINEARSPLLPQLGL |
| | | channel | | | GADYTYSNGYRDANGVNSNAT |
| | | protein TolC | | | SASLQLTQTLFDMSKWRALTLQ |
| | | | | | EKSAGIQDVTFQTDQQTLILNT |
| | | | | | ASAYFKVLNAIDVLSYTQAQKE |
| | | | | | AIYRQLDQTTQRFNVGLVAITD |
| | | | | | VQNARSQYDTVLANEVTARND |
| | | | | | LDNAVEELRQVIGNYYPELASL |
| | | | | | NVDSFKTDKPQAVNALLKEAE |
| | | | | | NRNLTLLQARLSQDLAREQIRQ |
| | | | | | AQDGHLPTLDLTASTGVSDTSY |
| | | | | | SGSKTRGSAVGSQYDDSNMGQ |
| | | | | | NKVGLSFSLPIYQGGMVNSQVK |
| | | | | | QAQYNFVGASEQLESAHRSVV |
| | | | | | QTVRSSFNNINASISSINAYKQA |
| | | | | | VVSAQSSLDAMEAGYSVGTRTI |
| | | | | | VDVLDATTTLYNAKQQLANAR |
| | | | | | YNYLINQLNIKSALGTLNEQDL |
| | | | | | VALNNTLGKPISTSPEHVAPETP |
| | | | | | QMDANADGYAANATAPATQP |
| | | | | | ASVRSSSSNGKNPFRN |
| 1 | WP_043017643.1 | MULTISPECIES: | Citrobacter | 345 | MKKLLPILIGLSLSGFSTLSQAE |
| | | Eouter | | | NLMQVYQQARLSNPELRKSAA |
| | | membrane | | | DRDAAFEKINEARSPLLPQLGL |
| | | channel | | | GADYTYSNGYRDANGVNSNAT |
| | | protein TolC | | | SASLQLTQTLFDMSKWRTLTLQ |
| | | | | | EKSAGIQDVTYQTDQQTLILNT |
| | | | | | ATAYFKVLNAIDVLSYTQAQK |
| | | | | | DAVYRQLDQTTQRFNVGLVAIT |
| | | | | | DVQNARSQYDTVLANEVTARN |
| | | | | | NLDNAVEELRQVTGNYYPELA |
| | | | | | SLNVDGFKTNKPQAVNALLKE |
| | | | | | AENRNLTLLQARLSQDLAREQI |
| | | | | | RQAQDGHLPTLDLTASTGVSDT |
| | | | | | SYSGSKTRGATGSQYDDSNMG |
| | | | | | QNKIGLSFSLPLYQGGMVNSQV |
| | | | | | KQAQYNFVGASEQLESAHRSV |
| | | | | | VQTVRSSFNNINASISSINAYKQ |
| | | | | | AVVSAQSSLDAMEAGYSVGTR |
| | | | | | TIVDVLDATTTLYNAKQQLAN |
| | | | | | ARYTYLINQLNVKSALGTLNEQ |
| | | | | | DLVALNNTLGKPIPTSPDSVAPQ |
| | | | | | NPQQDAAVNDFNSTGNMPAAQ |
| | | | | | PTAARSTSSNGNNPFRN |
| 1 | WP_086523458.1 | MULTISPECIES: | Citrobacter | 346 | MQMKKLLPILIGLSLSGFSTLSQ |
| | | outer | | | AENLMQVYQQARLSNPELRKS |
| | | membrane | | | AADRDAAFEKINEARSPLLPQL |
| | | channel | | | GLGADYTYSNGFRDANGVNSN |
| | | protein TolC | | | ATSASLSLTQTLFDMSKWRALT |
| | | | | | LQEKSAGIQDVTYQTDQQTLIL |
| | | | | | NTATAYFNVLNAIDVLSYTQAQ |
| | | | | | KDAVYRQLDQTTQRFNVGLVA |
| | | | | | ITDVQNARSQYDTVLANEVTAR |
| | | | | | NNLDNAVEQLRQVIGNYYPEL |
| | | | | | ASLNVDGFKTNKPQAVNSLLKE |
| | | | | | AENRNLTLLQARLSQDLAREQI |
| | | | | | RLAQDGHLPTLGLTASTGVSDT |
| | | | | | SYSGSKTNTSQYDDSNQGQNKI |
| | | | | | GLNFSLPLYQGGMVNSQVKQA |
| | | | | | QYNFVGASEQLESAHRSVVQT |
| | | | | | VRSSFNNINASISSINAYKQAVV |
| | | | | | SAQSSLDAMEAGYSVGTRTIVD |
| | | | | | VLDATTTLYNAKQQLANARYT |
| | | | | | YLINQLNIKSALGTLNEQDLVA |
| | | | | | LNNTLGKPISTAPDAVAPENTQ |
| | | | | | QDAAADGYTANSAAPAVKPAT |
| | | | | | ARTTSSSNGNNPFRN |
| 1 | WP_088583064.1 | MULTISPECIES: | Citrobacter | 347 | MQMKKLLPILIGLSLSGFTTLSQ |
| | | outer | | | AENLMQVYQQARLSNPELRKS |
| | | membrane | | | AADRDAAFEKINEARSPLLPQL |
| | | channel | | | GLGADYTYSNGYRDANGVNSN |
| | | protein TolC | | | ATSASLQLTQTLFDMSKWRALT |
| | | | | | LQEKAAGIQDVTYQTDQQTLIL |
| | | | | | NTATAYFNVLNAIDVLSYTQAQ |
| | | | | | KDAVYRQLDQTTQRFNVGLVA |
| | | | | | ITDVQNARSQYDTVLANEVTAR |
| | | | | | NNLDNAVEQLRQVIGNYYPEL |
| | | | | | ASLNVDGFKTNKPQAVNALLK |
| | | | | | EAENRNLSLLQARLSQDLAREQ |
| | | | | | IRLAQDGHLPTLGLTASTGVSD |
| | | | | | TSYNGSKTNTSQYDDSNMGQN |
| | | | | | KIGLNFSLPLYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQQLANARY |
| | | | | | TYLINQLNVKSALGTLNEQDLV |
| | | | | | ALNNTLGKPIPTSPDNVAPQNP |
| | | | | | QQDAAVNDFNSNGNMPAAQPT |
| | | | | | AARSTSSNGNNPFRN |
| 1 | WP_061076274.1 | outer | Citrobacter | 348 | MKKLLPILIGLSLSGFSTLSQAE |
| | | membrane | amalonaticus | | NLMQVYQQARLSNPELRKSAA |
| | | channel | | | DRDAAFEKINEARSPLLPQLGL |
| | | protein TolC | | | GADYTYSNGYRDANGVNSNAT |
| | | | | | SASLQLTQTLFDMSKWRALTLQ |
| | | | | | EKSAGIQDVTFQTDQQTLILNT |
| | | | | | ASAYFKVLNAIDVLSYTQAQKE |
| | | | | | AIYRQLDQTTQRFNVGLVAITD |
| | | | | | VQNARSQYDTVLANEVTARND |
| | | | | | LDNAVEELRQVTGNYYPELASL |
| | | | | | NVDSFKTDKPQAVNALLKEAE |
| | | | | | NRNLTLLQARLSQDLAREQIRQ |
| | | | | | AQDGHLPTLDLTASTGVSDTSY |
| | | | | | SGSKTRGATGSQYDDSNMGQN |
| | | | | | KIGLSFSLPIYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQQLANARY |
| | | | | | NYLINQLNIKSALGTLNEQDLV |
| | | | | | ALNNTLGKPISTSPEHVAPETPQ |
| | | | | | MDANADGYATNAAAPAAQPA |
| | | | | | SARSSSSNGNNPFRN |
| 1 | NP_819109.1 | type I | Coxiella burnetii | 349 | MKKKFLVFVLILFSLVAAPLLY |
| | | secretion outer | RSA 493 | | AEDLVQVYCQALASDPTFQKA |
| | | membrane | | | HADWLSARQNLPIAMSGTGTP |
| | | protein | | | GSGLFPYVDITAGLDRTFQRIEA |
| | | | | | GSSSVSGYFNQHNYQVTVTQPI |
| | | | | | FNYATWKAISSASFSVKAATAT |
| | | | | | YIEAAQDLIFRTAKAYFDVLDA |
| | | | | | YDQLQFTLAQKESFYHQLVTA |
| | | | | | QEKFKVGLIAITGVYDAQASYD |
| | | | | | QAIAQEIQDRNNLDNQLENLRA |
| | | | | | ITGQEYRSLTSLKKSIPLVIPHPR |
| | | | | | NIDAWTAVAERQSFAIQSALYT |
| | | | | | MLAQRETVKETAAQRYPTLTG |
| | | | | | TFSYGAQQRGFPPSLSGPPTTGE |
| | | | | | FLDTTTTTATAGLNLNFPVFQG |
| | | | | | GFVTHSTRQEEYNYLSASDQLN |
| | | | | | FTHRDVVRQTRQAYLGVDSGIS |
| | | | | | KIRADRQAIISAQNKLEATQAG |
| | | | | | YVVGTRTMVDVLDAVTSLYQA |
| | | | | | QQQWATDRYSYIINIITLKQQA |
| | | | | | GTLCPHDLAQINTWLGKAVRF |
| | | | | | DVEKPVNAKVFTPSTTLPHTVK |
| | | | | | HVHSPKGVQRATRSHRPLIKAS |
| | | | | | RHHTSPVHSASHIETVHAHYTIQ |
| | | | | | LFASRTLAEATAFKNKHRLHDL |
| | | | | | RIIHQNGWYKVLSGHYSTRQAA |
| | | | | | TIALHRLPSSLQKLKPWVVRVP |
| | | | | | KVQSTSIKPLSLQKKATHLPPPR |
| 1 | WP_007778110.1 | outer | Cronobacter | 350 | MKKLLPILIGLSLTGFSAMSQAE |
| | | membrane | malonaticus | | NLLQVYQQARLSNPDLRSSAAD |
| | | channel | | | RDAAFEKINEARSPLLPQLGLG |
| | | protein TolC | | | ADYTYTNGFRDNDGVDNTVKS |
| | | | | | ASLQLTQTIFDMSKWRALTLQE |
| | | | | | KTAGIQDVTYQTDQQTLMLNT |
| | | | | | ATAYFQVLSAIDALSYTEAQKQ |
| | | | | | AIYRQLDQTTQRFNVGLVAITD |
| | | | | | VQNARAQYDNVLANEVTARN |
| | | | | | NLDNALEQLRQVTGNYYPQLA |
| | | | | | SLNVDNFKTTKPAAINALLKEA |
| | | | | | EQRNLTLLQARLSQDLAREQIR |
| | | | | | YAETGHMPTLGLTASSSVSDTD |
| | | | | | YSGSKTSGAAASRYADSKIGQN |
| | | | | | SIGLSFNLPLYSGGSVTSQVKQA |
| | | | | | QYSFVGASEKLESAHRNVVQT |
| | | | | | VRSSYNNVNASISSIKAYEQAV |
| | | | | | VSAQSSLDAMEAGYSVGTRTIV |
| | | | | | DVLDATTTLYNAKQQLSSARY |
| | | | | | NYLINQLNIKSALGTLNEQDLV |
| | | | | | ALNNSLGKPVSTAPESVAPENP |
| | | | | | EQDAAVNNMANGGGSAPAMQ |
| | | | | | PAAATRSGNSNSGNPFRQ |
| 1 | WP_042868016.1 | MULTISPECIES: | Dickeya | 351 | MKKLLPLLISLGLVGFSAASQA |
| | | outer | | | EDLLQVYQQAKTTNPDLRSAA |
| | | membrane | | | ANRDAAFEKINEARSSLLPQLG |
| | | channel | | | LGADYTYNSGYRDRKDVNNNV |
| | | protein TolC | | | TSASLQLTQSIFNMSLWRSLTLQ |
| | | | | | EKQAGIQDVSYQTSQQTLLLNT |
| | | | | | ATAYFNVLRTIDALSYVSAQKK |
| | | | | | AIYRELDQTTQRFNVGLVAITD |
| | | | | | VQNARAQYDSVLANEVTARNN |
| | | | | | LDNAMENLRQVTGQFYPQLAA |
| | | | | | LNIEHFSQQKPDAVNGLLKEAE |
| | | | | | NRNLSLLSARLSQDLAREQIRSA |
| | | | | | ESGHLPTLNLTASTGVTNTNYS |
| | | | | | GSRVAASNLTDTYVGQNQVGL |
| | | | | | SLSVPIYSGGGTSSKVKQAQYSF |
| | | | | | VASSENLESAHRSVIQTLRSAH |
| | | | | | NNISASISSVNAYKQAVVSAQSS |
| | | | | | LDAMEAGYQVGTRTIVDVLDA |
| | | | | | TSTLYNAKQQLSGARYDYLINQ |
| | | | | | LNLKQALGTLNEDDLRTLNAM |
| | | | | | LGKPVPTSAAIGDDTPVPAATP |
| | | | | | VSVTTTTKARP |
| 1 | WP_022631861.1 | outer | Dickeya solani | 352 | MKKLLPLLISLGLGGFSAVSQA |
| | | membrane | | | EDLLQVYQQAKTTNPDLRSAA |
| | | channel | | | ANRDAAFEKINEARSSLLPQLG |
| | | protein TolC | | | LGADYTYNSGYRDRRDINSNAT |
| | | | | | SASLQLTQSIFNMSLWRALTLQ |
| | | | | | EKQAGIQDVSYQTSQQTLLLNT |
| | | | | | ATAYFSVLRAIDSLSYVSAQKQ |
| | | | | | SIYRELDQTTQRFNVGLVAITD |
| | | | | | VQNARAQYDSVLASEVTARNN |
| | | | | | LDNAMETLRQVTGQFYPQLAG |
| | | | | | LNIDRFSPQKPDAVNGLLKEAE |
| | | | | | NRNLSLLSARLSQDLAREQIRSA |
| | | | | | ESGHLPTLNLTASTGVTNTDYS |
| | | | | | GSRAVGDSFNNTYVGQNQVGL |
| | | | | | SFSLPIYSGGGTSSRVKQAQHSF |
| | | | | | VASSESMESAHRSVIQTLRSAH |
| | | | | | NNISASISSVNAYKQAVISAQSS |
| | | | | | LDAMEAGYQVGTRTIVDVLDA |
| | | | | | TTTLYNAKQQLSGARYDYLINQ |
| | | | | | LNLKQALGTLNEDDLRTLNAM |
| | | | | | LGKPVSTSAAIGDDTPAPATTPA |
| | | | | | AAKTAAKAKP |
| 1 | WP_034163047.1 | MULTISPECIES: | Edwardsiella | 353 | MKKLLPLLIGLSLSGFSALSQAE |
| | | outer | | | NLMQVYQQAKESNPDLRQAAA |
| | | membrane | | | NRDAAFEKINESRSPLLPQLGLG |
| | | channel | | | ADYTYTNGFRDSSGLNSNNYG |
| | | protein TolC | | | ATLALTQTIFDMSKWRQLSLTE |
| | | | | | KQAGIQDVSYQSSEQTLMLNTA |
| | | | | | TAYFNVLRAIDQLTYTEANKQA |
| | | | | | IYNQLDQTTQRFNVGLVAITDV |
| | | | | | QNARAQYDQVLAQEVSARNNL |
| | | | | | DNSLEALRQITGQYYPQLSALNI |
| | | | | | DRFSTKRPDDVKALLQEAEKRN |
| | | | | | LQLLSARLSQDLARQQIKYAET |
| | | | | | GYMPTLNLTASTGVSNTDYNSL |
| | | | | | SNAQKETLRGGNGSSYQGQNT |
| | | | | | VGLSFNLPLYSGGATNSQVKQA |
| | | | | | QYNFVGASEALDSAHRSVVQN |
| | | | | | VRSSFNNISAAISGVSAYKQAV |
| | | | | | VSAQSSLDATQAGYQVGTRTIV |
| | | | | | DVLNATTALYNAKQNLANARY |
| | | | | | DYLISQLNIKYALGTLNQNDLL |
| | | | | | ALNGDLSKPVPTAADSIAPDDA |
| | | | | | AQAAKANGPIPGGGVR |
| 1 | WP_045427820.1 | MULTISPECIES: | Edwardsiella | 354 | MKKLLPLLIGLSLSGFSALSQAE |
| | | outer | | | NLMQVYQQAKESNPDLRQAAA |
| | | membrane | | | NRDAAFEKINESRSPLLPQLGLG |
| | | channel | | | ADYTYTNGFRDSSGLNSNNYG |
| | | protein TolC | | | ATLALTQTIFDMSKWRQLSLTE |
| | | | | | KQAGIQDVSYQSSEQTLMLNTA |
| | | | | | TAYFNVLRAIDQLTYTEANKQA |
| | | | | | IYNQLDQTTQRFNVGLVAITDV |
| | | | | | QNARAQYDQVLAQEVSARNNL |
| | | | | | DNSLEALRQITGQYYPQLSTLNI |
| | | | | | DRFSTKRPDDVKALLQEAEKRN |
| | | | | | LQLLSARLSQDLARQQIKYAET |
| | | | | | GYMPTLNLTASTGVSNTDYNSL |
| | | | | | SNAQKETLRGGNGSSYQGQNT |
| | | | | | VGLSFNLPLYSGGATNSQVKQA |
| | | | | | QYNFVGASEALDSAHRSVVQN |
| | | | | | VRSSFNNISAAISGVSAYKQAV |
| | | | | | VSAQSSLDATQAGYQVGTRTIV |
| | | | | | DVLNATTALYNAKQNLANARY |
| | | | | | DYLISQLNIKYALGTLNQNDLL |
| | | | | | ALNGDLSKPVPTAADSIAPDDA |
| | | | | | AQAAKANGPIPGGSVR |
| 1 | WP_035608255.1 | outer | Edwardsiella | 355 | MKKLLPLLIGLSLSGFSALSQAE |
| | | membrane | ictaluri | | NLMQVYQQAKESNPDLRQAAA |
| | | channel | | | NRDAAFEKINESRSPLLPQLGLG |
| | | protein TolC | | | ADYTYTNGFRDSSGLNSNNYG |
| | | | | | ATLALTQTIFDMSKWRQLSLTE |
| | | | | | KQAGIQDVSYQSSEQTLMLNTA |
| | | | | | TAYFNVLRAIDQLTYTEANKQA |
| | | | | | IYNQLDQTTQRFKVGLVAITDV |
| | | | | | QNARAQYDQVLAQEVSARNNL |
| | | | | | DNSLEALRQITGQYYPQLSALNI |
| | | | | | DRFSTKRPDDVKALLQEAEKRN |
| | | | | | LQLLSARLSQDLARQQIKYAET |
| | | | | | GYMPTLNLTASTGVSNTDYNSL |
| | | | | | SNAQKETLRGGNGSSYQGQNT |
| | | | | | VGLSFNLPLYSGGATNSQVKQA |
| | | | | | QYNFVGASEALDSAHRGVVQN |
| | | | | | VRSSFNNISAAISSVSAYKQAVV |
| | | | | | SAQSSLDATQAGYQVGTRTIVD |
| | | | | | VLNATTALYNAKQNLANARYD |
| | | | | | YLISQLNIKYALGTLNQNDLLM |
| | | | | | LNGDLSKTVPTAADSIAPDDAA |
| | | | | | QAAKANGPIPGGGVR |
| 1 | WP_023314865.1 | MULTISPECIES: | Enterobacter | 356 | MKKLLPILIGLSLTGFSAMSQAE |
| | | outer | | | NLLQVYQQARLGNPDLRKSAA |
| | | membrane | | | DRDAAFEKINEARSPLLPQLGL |
| | | channel | | | GADYTYTNGFRDNNGINSNATS |
| | | protein TolC | | | ASLQLTQTLFDMSKWRELSLQE |
| | | | | | KSAGIQDVTYQTDQQTLILNTA |
| | | | | | TAYFNVLSAIDALSYTEAQKQA |
| | | | | | IYRQLDQTTQRFNVGLVAITDV |
| | | | | | QNARSQYDTVLANEVTARNNL |
| | | | | | DNALEALRQVIGNYYPELASL |
| | | | | | NVDSFKTDKPQAVNALLKEAE |
| | | | | | NRNLTLLQARLSQDLAREQIRL |
| | | | | | AQDGHLPTLSLSASTGVSDTSY |
| | | | | | SGSKTNTAQYDDNNMGQNKV |
| | | | | | GLSFSLPLYQGGMVNSQVKQA |
| | | | | | QYNFVGASEQLESAHRNVVQT |
| | | | | | VRSSFNNVNASISSINAYKQAV |
| | | | | | VSAQSSLDAMEAGYSVGTRTIV |
| | | | | | DVLDATTTLYNAKQQLSSARY |
| | | | | | QYLINQLNIKQALGTLNEQDLQ |
| | | | | | MLNSTLGKPVSTSPDSVAPENP |
| | | | | | QQDAAVDNFTANSSTPVAQPA |
| | | | | | AARSTSPASSGTNPFRN |
| 1 | WP_032659905.1 | MULTISPECIES: | Enterobacter | 357 | MKKLLPILIGLSLTGFSAMSQAE |
| | | outer | | | NLLQVYQQARLGNPDLRKSAA |
| | | membrane | | | DRDAAFEKINEARSPLLPQLGL |
| | | channel | | | GADYTYSNGFRDANGVNSNAT |
| | | protein TolC | | | SASLQLTQTLFDMSKWRALTLQ |
| | | | | | EKSAGIQDVTYQTDQQTLILNT |
| | | | | | ATAYFNVLSAIDALSYTEAQKQ |
| | | | | | AIYRQLDQTTQRFNVGLVAITD |
| | | | | | VQNARSQYDTVLANEVTARNN |
| | | | | | LDNALESLRQVIGNYYPELASL |
| | | | | | NVDSFKTDKPQAVNALLKEAE |
| | | | | | NRNLSLLQARLSQDLAREQIRQ |
| | | | | | AQDGHLPTLSLSASTGVSDTTY |
| | | | | | SGSKTNTSQYDDSNMGQNKVG |
| | | | | | LSFSLPLYQGGQVNSQVKQAQ |
| | | | | | YNFVGASEQLESAHRNVVQTV |
| | | | | | RSSFNNVNASISSINAYKQAVVS |
| | | | | | AQSSLDAMEAGYSVGTRTIVDV |
| | | | | | LDATTTLYNAKQQLSSARYQY |
| | | | | | LINQLNIKQALGTLNEQDLQML |
| | | | | | NSALGKPVSTSPDSVAPENPEQ |
| | | | | | VAAVDNFNANSNTPAAQPAAA |
| | | | | | RTTTSASKGNNPFRN |
| 1 | WP_023306968.1 | MULTISPECIES: | Enterobacter | 358 | MKKLLPILIGLSLTGFSAMSQAE |
| | | outer | cloacae complex | | NLLQVYQQARLGNPDLRKSAA |
| | | membrane | | | DRDAAFEKINEARSPLLPQLGL |
| | | channel | | | GADYTYTNGFRDNNGIDSNATS |
| | | protein TolC | | | ASLQLTQTLFDMSKWRELSLQE |
| | | | | | KSAGIQDVTYQTDQQTLILNTA |
| | | | | | TAYFQVLSAIDALSYTEAQKQA |
| | | | | | IYRQLDQTTQRFNVGLVAITDV |
| | | | | | QNARSQYDTVLANEVTARNNL |
| | | | | | DNALEALRQVTGNYYPELASL |
| | | | | | NVDSFKTDKPQAVNALLKEAE |
| | | | | | NRNLTLLQARLSQDLAREQIRL |
| | | | | | AQDGHLPTLSLSASTGVSDTSY |
| | | | | | SGSKTTSQAYDDSNVGQNKVG |
| | | | | | LSFSLPLYQGGMVNSQVKQAQ |
| | | | | | YNFVGASEQLESAHRNVVQTV |
| | | | | | RSSFNNVNASISSINAYKQAVVS |
| | | | | | AQSSLDAMEAGYSVGTRTIVDV |
| | | | | | LDATTTLYNAKQQLSSARYQY |
| | | | | | LINQLNIKQALGTLNEQDLQML |
| | | | | | NSTLGKPVSTSPDSVAPENPEQ |
| | | | | | DAAVDNFNANGSAPAAQPAAA |
| | | | | | RSTSPASSGTNPFRN |
| 1 | WP_032642076.1 | MULTISPECIES: | Enterobacter | 359 | MKKLLPILIGLSLTGFSAMSQAE |
| | | outer | cloacae complex | | NLLQVYQQARLGNPDLRKSAA |
| | | membrane | | | DRDAAFEKINEARSPLLPQLGL |
| | | channel | | | GADYTYSNGFRDANGVNSNAT |
| | | protein TolC | | | SASLQLTQTLFDMSKWRALTLQ |
| | | | | | EKSAGIQDVTYQTDQQTLILNT |
| | | | | | ATAYFNVLSAIDALSYTEAQKQ |
| | | | | | AIYRQLDQTTQRFNVGLVAITD |
| | | | | | VQNARSQYDTVLANEVTARNN |
| | | | | | LDNALESLRQVIGNYYPELASL |
| | | | | | NVDSFKTDKPQAVNALLKEAE |
| | | | | | NRNLSLLQARLSQDLAREQIRQ |
| | | | | | AQDGHLPTLSLSASTGVSDTTY |
| | | | | | SGSKTNTSQYDDSNLGQNKVG |
| | | | | | LSFSLPLYQGGQVNSQVKQAQ |
| | | | | | YNFVGASEQLESAHRNVVQTV |
| | | | | | RSSFNNVNASISSINAYKQAVVS |
| | | | | | AQSSLDAMEAGYSVGTRTIVDV |
| | | | | | LDATTTLYNAKQLLSSARYQYL |
| | | | | | INQLNIKQALGTLNEQDLQMLN |
| | | | | | SALGKPVSTSPDSVAPENPQQV |
| | | | | | AAVDNFNANGSAPAAQPAAAR |
| | | | | | TTAPASKGNNPFRN |
| 1 | WP_087855185.1 | MULTISPECIES: | Enterobacter | 360 | MKKLLPILIGLSLTGFSAMSQAE |
| | | outer | cloacae complex | | NLLQVYQQARLGNPDLRKSAA |
| | | membrane | | | DRDAAFEKINEARSPLLPQLGL |
| | | channel | | | GADYTYSNGFRDANGVNSNAT |
| | | protein TolC | | | SASLQLTQTLFDMSKWRALTLQ |
| | | | | | EKSAGIQDVTYQTDQQTLILNT |
| | | | | | ATAYFNVLSAIDALSYTEAQKQ |
| | | | | | AIYRQLDQTTQRFNVGLVAITD |
| | | | | | VQNARSQYDTVLANEVTARNN |
| | | | | | LDNALESLRQVTGNYYPELASL |
| | | | | | NVDSFKTDKPQAVNALLKEAE |
| | | | | | NRNLTLLQARLSQDLAREQIRQ |
| | | | | | AQDGHLPTLSLSASTGVSDTTY |
| | | | | | SGSKTNSAQYDDSNMGQNKVG |
| | | | | | LSFSLPLYQGGQVNSQVKQAQ |
| | | | | | YNFVGASEQLESAHRNVVQTV |
| | | | | | RSSFNNVNASISSINAYKQAVVS |
| | | | | | AQSSLDAMEAGYSVGTRTIVDV |
| | | | | | LDATTTLYNAKQQLSSARYQY |
| | | | | | LINQLNIKQALGTLNEQDLQML |
| | | | | | NSALGKPVSTSPDSVAPENPEQ |
| | | | | | VAAVDNFNANSTTPAAQPAAA |
| | | | | | RTTAPASKGNNPFRN |
| 1 | WP_149121178.1 | outer | Enterobacter sp. | 361 | MKKLLPILIGLSLTGFSAMSQAE |
| | | membrane | LU1 | | NLLQVYQQARLGNPDLRKSAA |
| | | channel | | | DRDAAFEKINEARSPLLPQLGL |
| | | protein TolC | | | GADYTYTNGFRDNNGIDSNATS |
| | | | | | ASLQLTQTLFDMSKWRELSLQE |
| | | | | | KSAGIQDVTYQTDQQTLILNTA |
| | | | | | TAYFQVLSAIDALSYTEAQKQA |
| | | | | | VYRQLDQTTQRFNVGLVAITDV |
| | | | | | QNARSQYDTVLANEVTARNNL |
| | | | | | DNALEALRQVIGNYYPELASL |
| | | | | | NVDSFKTDKPQAVNALLKEAE |
| | | | | | NRNLTLLQARLSQDLAREQIRL |
| | | | | | AQDGHLPTLSLSASTGVSDTSY |
| | | | | | SGSKTTSQAYDDSNVGQNKVG |
| | | | | | LSFSLPLYQGGMVNSQVKQAQ |
| | | | | | YNFVGASEQLESAHRNVVQTV |
| | | | | | RSSYNNVNASISSINAYKQAVV |
| | | | | | SAQSSLDANEAGYSVGTRTIVD |
| | | | | | VLDATTTLYNAKQQLSSARYQ |
| | | | | | YLINQLNIKQALGTLNEQDLQM |
| | | | | | LNSTLGKPVPTSPDSVAPENPQ |
| | | | | | QDAAVDNFTPNSSAPLAQPAAA |
| | | | | | RSTATASSGTNPFRH |
| 1 | WP_072569740.1 | outer | Enterobacter sp. | 362 | MKKLLPILIGLSLAGFSAMSQAE |
| | | membrane | SA187 | | NLLQVYKQARLSNPELRRSAAD |
| | | channel | | | RDAAFEKINEARSPLLPQLGLG |
| | | protein TolC | | | ADYTYSNGFRDSNGVNSNATS |
| | | | | | ASLQLTQTLFDMSKWRALTLQ |
| | | | | | EKAAGIQDVTFQTDQQTLILNT |
| | | | | | ATAYFNVLSAIDSLSYTEAQKQ |
| | | | | | AIYRQLDQTTQRFNVGLVAITD |
| | | | | | VQNARSQYDTVLANEVTARNN |
| | | | | | LDNAVEQLRQVIGNYYPELAS |
| | | | | | LNVDNFKTDKPQPVNALLKEA |
| | | | | | ENRNLSLLQARLNQDLAREQIR |
| | | | | | QAQDGHLPTLDLTASTGVSDTS |
| | | | | | YSGSKTNNAQFDDSNVGQNKV |
| | | | | | GLNFSLPIFQGGMVNSQVKQAQ |
| | | | | | YNFVGASEQLETAHRSVVQNV |
| | | | | | RSSFNNVNASISSIKAYEQAVVS |
| | | | | | AQSSLDASEAGYSVGTRTIVDV |
| | | | | | LDATTALYNAKQQLSSARYNY |
| | | | | | LINQLNIKSALGTLNEQDLLALN |
| | | | | | STLGKPTSTTPEAVAPENPQQD |
| | | | | | ARVQNIAADAQSATTADAPVA |
| | | | | | TNSSRSGANPFSR |
| 1 | WP_104950097.1 | outer | Enterobacter sp. | 363 | MKKLLPILIGLSLTGFSAMSQAE |
| | | membrane | SGAir0187 | | NLLQVYQQARLGNPDLRKSAA |
| | | channel | | | DRDAAFEKINEARSPLLPQLGL |
| | | protein TolC | | | GADYTYSNGFRDANGVNSNAT |
| | | | | | SASLQLTQTLFDMSKWRALTLQ |
| | | | | | EKTAGIQDVTYQTDQQTLILNT |
| | | | | | ATAYFNVLSAIDALSYTEAQKQ |
| | | | | | AIYRQLDQTTQRFNVGLVAITD |
| | | | | | VQNARSQYDTVLANEVTARNN |
| | | | | | LDNALESLRQVIGNYYPELASL |
| | | | | | NVDSFKTDKPQAVNALLKEAE |
| | | | | | NRNLTLLQARLSQDLAREQIRQ |
| | | | | | AQDGHLPTLSLSASTGVSDTTY |
| | | | | | SGSKTNSAQYDDSNMGQNKVG |
| | | | | | LSFSLPLYQGGQVNSQVKQAQ |
| | | | | | YNFVGASEQLESAHRNVVQTV |
| | | | | | RSSFNNVNASISSINAYKQAVVS |
| | | | | | AQSSLDAMEAGYSVGTRTIVDV |
| | | | | | LDATTTLYNAKQQLSSARYQY |
| | | | | | LINQLNIKQALGTLNEQDLQML |
| | | | | | NSALGKPVSTSPDSVAPENPDQ |
| | | | | | VAAVDNFNANGSTPAAQPAAE |
| | | | | | RTTAPASKSNNPFRN |
| 1 | WP_033146662.1 | MULTISPECIES: | Enterobacterales | 364 | MKKLLPILIGLSLTGFSAMSQAE |
| | | outer | | | NLLQVYQQARLGNPDLRKSAA |
| | | membrane | | | DRDAAFEKINEARSPLLPQLGL |
| | | channel | | | GADYTYSNGFRDANGVNSNAT |
| | | protein TolC | | | SASLQLTQTLFDMSKWRALTLQ |
| | | | | | EKSAGIQDVTYQTDQQTLILNT |
| | | | | | ATAYFNVLSAIDALSYTEAQKQ |
| | | | | | AIYRQLDQTTQRFNVGLVAITD |
| | | | | | VQNARSQYDTVLANEVTARNN |
| | | | | | LDNALESLRQVIGNYYPELASL |
| | | | | | NVDSFKTDKPQAVNALLKEAE |
| | | | | | NRNLSLLQARLSQDLAREQIRQ |
| | | | | | AQDGHLPTLSLSASTGVSDTSY |
| | | | | | SGSKTNTSQYDDSNMGQNKVG |
| | | | | | LSFSLPLYQGGQVNSQVKQAQ |
| | | | | | YNFVGASEQLESAHRNVVQTV |
| | | | | | RSSFNNVNASISSINAYKQAVVS |
| | | | | | AQSSLDAMEAGYSVGTRTIVDV |
| | | | | | LDATTTLYNAKQQLSSARYQY |
| | | | | | LINQLNIKQALGTLNEQDLQML |
| | | | | | NSALGKPVSTSPDSVAPENPEQ |
| | | | | | VAAVDNFNANSSTPAAQPAAA |
| | | | | | RTTTSASKGNNPFRN |
| 1 | WP_000735289.1 | MULTISPECIES: | Enterobacteriaceae | 365 | MKKLLPILIGLSLSGFSSLSQAE |
| | | outer | | | NLMQVYQQARLSNPELRKSAA |
| | | membrane | | | DRDAAFEKINEARSPLLPQLGL |
| | | channel | | | GADYTYSNGYRDANGINSNAT |
| | | protein TolC | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNTLLKEAEK |
| | | | | | RNLSLLQARLSQDLAREQIRQA |
| | | | | | QDGHLPTLDLTASTGISDTSYSG |
| | | | | | SKTRGAAGTQYDDSNMGQNK |
| | | | | | VGLSFSLPIYQGGMVNSQVKQA |
| | | | | | QYNFVGASEQLESAHRSVVQT |
| | | | | | VRSSFNNINASISSINAYKQAVV |
| | | | | | SAQSSLDAMEAGYSVGTRTIVD |
| | | | | | VLDATTTLYNAKQELANARYN |
| | | | | | YLINQLNIKSALGTLNEQDLLAL |
| | | | | | NNALSKPVSTNPENVAPQTPEQ |
| | | | | | NAIADGYAPDSPAPVVQQTSAR |
| | | | | | TTTSNGHNPFRN |
| 1 | WP_024176395.1 | MULTISPECIES: | Enterobacteriaceae | 366 | MKKLLPILIGLSLSGFSSLSQAE |
| | | outer | | | NLMQVYQQARLSNPELRKSAA |
| | | membrane | | | DRDAAFEKINEARSPLLPQLGL |
| | | channel | | | GADYTYSNGYRDANGINSNAT |
| | | protein TolC | | | SASLQLTQSIFDMSKWRALTLQ |
| | | | | | EKAAGIQDVTHQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNALLKEAE |
| | | | | | KRNLSLLQARLSQDLAREQIRQ |
| | | | | | AQDGHLPTLDLTASTGISDTSYS |
| | | | | | GSKTRGAAGTQYDDSNMGQN |
| | | | | | KVGLSFSLPIYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQELANARY |
| | | | | | NYLINQLNIKSALGTLNEQDLL |
| | | | | | ALNNALSKPVSTNPENVAPQTP |
| | | | | | EQNAIADGYAPDSPAPVVQQTS |
| | | | | | ARTTTSNGHNPFRN |
| 1 | WP_032937374.1 | MULTISPECIES: | Enterobacteriaceae | 367 | MKKLLPILIGLSLSGFSTLSQAE |
| | | outer | | | NLMQVYQQARLSNPELRKSAA |
| | | membrane | | | DRDAAFEKINEARSPLLPQLGL |
| | | channel | | | GADYTYSNGYRDANGVNSNAT |
| | | protein TolC | | | SASLQLTQTLFDMSKWRALTLQ |
| | | | | | EKSAGIQDVTYQTDQQTLILNT |
| | | | | | ATAYFNVLNAIDVLSYTQAQK |
| | | | | | DAVYRQLDQTTQRENVGLVAIT |
| | | | | | DVQNARSQYDTVLANEVTARN |
| | | | | | NLDNAVEQLRQVIGNYYPELA |
| | | | | | SLNVDGFKTNKPQAVNALLKE |
| | | | | | AENRNLTLLQARLSQDLAREQI |
| | | | | | RQAQDGHLPTLDLTASTGVSDT |
| | | | | | SYSGSKTHGGTTGNQYDDSNM |
| | | | | | GQNKIGLSFSLPLYQGGMVNSQ |
| | | | | | VKQAQYNFVGASEQLESAHRS |
| | | | | | VVQTVRSSFNNINASISSINAYK |
| | | | | | QAVVSAQSSLDAMEAGYSVGT |
| | | | | | RTIVDVLDATTTLYNAKQQLAN |
| | | | | | ARYTYLINQLNVKSALGTLNEQ |
| | | | | | DLVALNNTLGKPVSTTPDTVAP |
| | | | | | QNPQQDAAVNDENGTGNLPAA |
| | | | | | QPTAARSTSSNGNNPFRN |
| 1 | WP_054411816.1 | MULTISPECIES: | Escherichia | 368 | MKKLLPILIGLSLSGFSTLSQAE |
| | | outer | | | NLMQVYQQARLSNPELRKSAA |
| | | membrane | | | DRDAAFEKINEARSPLLPQLGL |
| | | channel | | | GADYTYSNGYRDANGINSNAT |
| | | protein TolC | | | SASLQLTQSIFDMSKWRALTLQ |
| | | | | | EKAAGIQDVTYQTDQQALILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNTLLKEAEK |
| | | | | | RNLTLLQARLSQDLAREQIRLA |
| | | | | | QDGHLPTLNLTASTGISDTSYSG |
| | | | | | SKTRGANSAQYDDSNMGQNKV |
| | | | | | GLSFSLPIYQGGMVNSQVKQAQ |
| | | | | | YNFVGASEQLESAHRSVVQTVR |
| | | | | | SSFNNINASISSINAYKQAVVSA |
| | | | | | QSSLDAMEAGYSVGTRTIVDVL |
| | | | | | DATTTLYNAKQELANARYNYLI |
| | | | | | NQLNIKSALGTLNEQDLLALNN |
| | | | | | ALSKPVSTNPENVAPQTPEQNAI |
| | | | | | ADGYTPDSPAPVVQPASVRTNT |
| | | | | | STGKNPFRN |
| 1 | WP_000735296.1 | outer | Escherichia | 369 | MKKLLPILIGLSLSGFSTLSQAE |
| | | membrane | albertii | | NLMQVYQQARLSNPELRKSAA |
| | | channel | | | DRDAAFEKINEARSPLLPQLGL |
| | | protein TolC | | | GADYTYSNGYRDANGINSNAT |
| | | | | | SASLQLTQSIFDMSKWRALTLQ |
| | | | | | EKAAGIQDVTYQTDQQALILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNTLLKEAEK |
| | | | | | RNLTLLQARLSQDLAREQIRQA |
| | | | | | QDGHLPTLNLTASTGISDTSYSG |
| | | | | | SKTRGANSAQYDDSNMGQNKV |
| | | | | | GLSFSLPIYQGGMVNSQVKQAQ |
| | | | | | YNFVGASEQLESAHRSVVQTVR |
| | | | | | SSFNNINASISSINAYKQAVVSA |
| | | | | | QSSLDAMEAGYSVGTRTIVDVL |
| | | | | | DATTTLYNAKQELANARYNYLI |
| | | | | | NQLNIKSALGTLNEQDLLALNN |
| | | | | | ALSKPVSTNPENVAPQTPEQNAI |
| | | | | | ADGYTPDSPAPVVQPASVRTNT |
| | | | | | STGKNPFRN |
| 1 | WP_025238316.1 | outer | Escherichia | 370 | MKKLLPILIGLSLSGFSTLSQAE |
| | | membrane | albertii | | NLMQVYQQARLSNPELRKSAA |
| | | channel | | | DRDAAFEKINEARSPLLPQLGL |
| | | protein TolC | | | GADYTYSNGYRDANGINSNAT |
| | | | | | SASLQLTQSIFDMSKWRALTLQ |
| | | | | | EKAAGIQDVTYQTDQQALILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNTLLKEAEK |
| | | | | | RNLTLLQARLSQDLAREQIRQA |
| | | | | | QDGHLPTLNLTASTGISDTSYSG |
| | | | | | SKTRGANSAQYDDSNMGQNKV |
| | | | | | GLSFSLPIYQGGMVNSQVKQAQ |
| | | | | | YNFVGASEQLESAHRSVVQTVR |
| | | | | | SSFNNINASISSINAYKQAVVSA |
| | | | | | QSSLDAMEAGYSVGTRTIVDVL |
| | | | | | DATTTLYNAKQELANARYNYLI |
| | | | | | NQLNIKSALGTLNEQDLLALNN |
| | | | | | ALSKPVSTNPENVAPQTPEQNAI |
| | | | | | ADGYTPDSPVPVVQPASVRTNT |
| | | | | | STGKNPFRN |
| 1 | WP_113650417.1 | outer | Escherichia | 371 | MKKLLPILIGLSLSGFSTLSQAE |
| | | membrane | albertii | | NLMQVYQQARLSNPELRKSAA |
| | | channel | | | DRDAAFEKINEARSPLLPQLGL |
| | | protein TolC | | | GADYTYSNGYRDANGINSNAT |
| | | | | | SASLQLTQSIFDMSKWRALTLQ |
| | | | | | EKAAGIQDVTYQTDQQALILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNTLLKEAET |
| | | | | | RNLTLLQARLSQDLAREQIRQA |
| | | | | | QDGHLPTLNLTASTGISDTSYSG |
| | | | | | SKTRGANSAQYDDSNMGQNKV |
| | | | | | GLSFSLPIYQGGMVNSQVKQAQ |
| | | | | | YNFVGASEQLESAHRSVVQTVR |
| | | | | | SSFNNINASISSINAYKQAVVSA |
| | | | | | QSSLDAMEAGYSVGTRTIVDVL |
| | | | | | DATTTLYNAKQELANARYNYLI |
| | | | | | NQLNIKSALGTLNEQDLLALNN |
| | | | | | ALSKPVSTNPENVAPQTPEQNAI |
| | | | | | ADGYTPDSPVPVVQPASVRTNT |
| | | | | | STGKNPFRN |
| 1 | WP_000735277.1 | outer | Escherichia coli | 372 | MKKLLPILIGLSLSGFSSLSQAE |
| | | membrane | | | NLMQVYQQARLSNPELRKSAA |
| | | channel | | | DRDAAFEKINEARSPLLPQLGL |
| | | protein TolC | | | GADYTYSNGYRDANGINSNAT |
| | | | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNALLKEAE |
| | | | | | KRNLSLLQARLSQDLAREQIRQ |
| | | | | | AQDGHLPTLDLTASTGISDTSYS |
| | | | | | GSKTRGAAGTQYDDSNMGQN |
| | | | | | KVGLSFSLPIYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQELANARY |
| | | | | | NYLINQLNIKSALGTLNEQDLL |
| | | | | | ALNNALSKPVSTNPENVAPQTP |
| | | | | | EQNAIADGYAPDSPAPVVQQTF |
| | | | | | ARTTTSNGHNPFRN |
| 1 | WP_000735279.1 | outer | Escherichia coli | 373 | MKKLLPILIGLSLSGFSSLSQAE |
| | | membrane | | | NLMQVYQQARLSNPELRKSAA |
| | | channel | | | DRDAAFEKINEARSPLLPQLGL |
| | | protein TolC | | | GADYTYSNGYRDANGINSNAT |
| | | | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNALLKEAE |
| | | | | | KRNLSLLQARLSQDLAREQIRQ |
| | | | | | AQDGHLPTLDLTASTGISDTSYS |
| | | | | | GSKTRGAAGTQYDDSNMGQN |
| | | | | | KVGLSFSLPIYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQELANARY |
| | | | | | NYLINQLNIKSALGTLNEQDLL |
| | | | | | ALNNALSKPVSTNPENVAPQTP |
| | | | | | EQNAIADGYAPDSPVPVVQQTS |
| | | | | | ARTTTSNGHNPFRN |
| 1 | WP_000735290.1 | outer | Escherichia coli | 374 | MKKLLPILIGLSLSGFSSLSQAE |
| | | membrane | | | NLMQVYQQARLSNPELRKSAA |
| | | channel | | | DRDAAFEKINEARSPLLPQLGL |
| | | protein TolC | | | GADYTYSNGYRDANGINSNAT |
| | | | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNTLLKEAEK |
| | | | | | RNLSLLQARLSQDLAREQIRQA |
| | | | | | QDGHLPTLDLTASTGISDTSYSG |
| | | | | | SKTRGATAGQYDDSNMGQNK |
| | | | | | VGLSFSLPIYQGGMVNSQVKQA |
| | | | | | QYNFVGASEQLESAHRSVVQT |
| | | | | | VRSSFNNINASISSINAYKQAVV |
| | | | | | SAQSSLDAMEAGYSVGTRTIVD |
| | | | | | VLDATTTLYNAKQELANARYN |
| | | | | | YLINQLNIKSALGTLNEQDLLAL |
| | | | | | NNALSKPVSTNPENVAPQTPEQ |
| | | | | | NAIADGYAPDSPAPVVQQTSAR |
| | | | | | TTTSNGHNPFRN |
| 1 | WP_000735293.1 | outer | Escherichia coli | 375 | MKKLLPILIGLSLSGFSSLSQAE |
| | | membrane | | | NLMQVYQQARLSNPELRKSAA |
| | | channel | | | DRDAAFEKINEARSPLLPQLGL |
| | | protein TolC | | | GADYTYSNGYRDANGINSNAT |
| | | | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYTELAAL |
| | | | | | NVENFKTDKPQPVNALLKEAE |
| | | | | | KRNLSLLQARLSQDLAREQIRQ |
| | | | | | AQDGHLPTLDLTASTGISDTSYS |
| | | | | | GSKTRGAAGTQYDDSNMGQN |
| | | | | | KVGLSFSLPIYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQELANARY |
| | | | | | NYLINQLNIKSALGTLNEQDLL |
| | | | | | ALNNALSKPVSTNPENVAPQTP |
| | | | | | EQNAIADGYAPDSPAPVVQQTS |
| | | | | | ARTTTSNGHNPFRN |
| 1 | WP_000735322.1 | outer | Escherichia coli | 376 | MKKLLPILIGLSLYGFSSLSQAE |
| | | membrane | | | NLMQVYQQARLSNPELRKSAA |
| | | channel | | | DRDAAFEKINEARSPLLPQLGL |
| | | protein TolC | | | GADYTYSNGYRDANGINSNAT |
| | | | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNALLKEAE |
| | | | | | KRNLSLLQARLSQDLAREQIRQ |
| | | | | | AQDGHLPTLDLTASTGISDTSYS |
| | | | | | GSKTRGAAGTQYDDSNMGQN |
| | | | | | KVGLSFSLPIYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQELANARY |
| | | | | | NYLINQLNIKSALGTLNEQDLL |
| | | | | | ALNNALSKPVSTNPENVAPQTP |
| | | | | | EQNAIADGYAPDSPAPVVQQTS |
| | | | | | ARTTTSNGHNPFRN |
| 1 | WP_050938914.1 | outer | Escherichia coli | 377 | MKKLLPILIGLSLSGFSSLSQAE |
| | | membrane | | | NLMQVYQQARLSNPELRKSAA |
| | | channel | | | DRDAAFEKINEARSPLLPQLGL |
| | | protein TolC | | | GADYTYSNGYRDANGINSNAT |
| | | | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNALLKEAE |
| | | | | | KRNLSLLQARLSQDLAREQIRQ |
| | | | | | AQDGHLPTLDLTASTGISDTSYS |
| | | | | | GSKTRGAAGTQYDDSNMGQN |
| | | | | | KVGLSFSLPIYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQELANARY |
| | | | | | NYLINQLNIKSALGSLNEQDLL |
| | | | | | ALNNALSKPVSTNPENVAPQTP |
| | | | | | EQNAIADGYAPDSPAPVVQQTS |
| | | | | | ARTTTSNGHNPFRN |
| 1 | WP_096220597.1 | outer | Escherichia coli | 378 | MKKLLPILIGLSLSGFSSLSQAE |
| | | membrane | | | NLMQVYQQARLSNPELRKSAA |
| | | channel | | | DRDAAFEKINEARSPLLPQLGL |
| | | protein TolC | | | GADYTYSNGYRDANGINSNAA |
| | | | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNALLKEAE |
| | | | | | KRNLSLLQARLSQDLAREQIRQ |
| | | | | | AQDGHLPTLDLTASTGISDTSYS |
| | | | | | GSKTRGAAGTQYDDSNMGQN |
| | | | | | KVGLSFSLPIYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQELANARY |
| | | | | | NYLINQLNIKSALGTLNEQDLL |
| | | | | | ALNNALSKPVSTNPENVAPQTP |
| | | | | | EQNAIADGYAPDSPAPVVQQTS |
| | | | | | ARTTTSNGHNPFRN |
| 1 | NP_311950.2 | transport | Escherichia coli | 379 | MKKLLPILIGLSLSGFSSLSQAE |
| | | channel | O157:H7 str. | | NLMQVYQQARLSNPELRKSAA |
| | | | Sakai | | DRDAAFEKINEARSPLLPQLGL |
| | | | | | GADYTYSNGYRDANGINSNAT |
| | | | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNALLKEAE |
| | | | | | KRNLSLLQARLSQDLAREQIRQ |
| | | | | | AQDGHLPTLDLTASSGISDTSYS |
| | | | | | GSKTRGAAGTQYDDSNMGQN |
| | | | | | KVGLSFSLPIYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQELANARY |
| | | | | | NYLINQLNIKSALGTLNEQDLL |
| | | | | | ALNNALSKPVSTNPENVAPQTP |
| | | | | | EQNAIADGYAPDSPAPVVQQTS |
| | | | | | ARTTTSNGHNPFRN |
| 1 | NP_417507.2 | outer | Escherichia coli | 380 | MKKLLPILIGLSLSGFSSLSQAE |
| | | membrane | str. K-12 substr. | | NLMQVYQQARLSNPELRKSAA |
| | | channel TolC | MG1655 | | DRDAAFEKINEARSPLLPQLGL |
| | | | | | GADYTYSNGYRDANGINSNAT |
| | | | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNALLKEAE |
| | | | | | KRNLSLLQARLSQDLAREQIRQ |
| | | | | | AQDGHLPTLDLTASTGISDTSYS |
| | | | | | GSKTRGAAGTQYDDSNMGQN |
| | | | | | KVGLSFSLPIYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQELANARY |
| | | | | | NYLINQLNIKSALGTLNEQDLL |
| | | | | | ALNNALSKPVSTNPENVAPQTP |
| | | | | | EQNAIADGYAPDSPAPVVQQTS |
| | | | | | ARTTTSNGHNPFRN |
| 1 | WP_000735267.1 | outer | Escherichia | 381 | MKKLLPILIGLSLSGFSALSQAE |
| | | membrane | fergusonii | | NLMQVYQQARLSNPELRKSAA |
| | | channel | | | DRDAAFEKINEARSPLLPQLGL |
| | | protein TolC | | | GADYTYSNGYRDANGINSNAT |
| | | | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NIDNFKTDKPQPVNALLKEAEK |
| | | | | | RNLSLLQARLSQDLAREQIRQA |
| | | | | | QDGHLPTLDLTASTGISDTSYSG |
| | | | | | SKTRGANSAQYDDSNMGQNKV |
| | | | | | GLSFSLPIYQGGMVNSQVKQAQ |
| | | | | | YNFVGASEQLESAHRSVVQTVR |
| | | | | | SSFNNINASISSINAYKQAVVSA |
| | | | | | QSSLDAMEAGYSVGTRTIVDVL |
| | | | | | DATTTLYNAKQQLANARYNYL |
| | | | | | INQLNIKSALGTLNEQDLIALNN |
| | | | | | ALSKPVSTNPENVAPQTPEQNAI |
| | | | | | ADGYAPDSPAPVVQQTSVRTSN |
| | | | | | GNNPFRN |
| 1 | WP_005503997.1 | outer | Grimontia | 382 | MKKLLPFFIGVALGTAAPLASA |
| | | membrane | hollisae | | DDLIQVYEQAKQSDPQLLQAA |
| | | channel | | | ATKDAAFAAVDTNRGTLLPQIN |
| | | protein TolC | | | LQAGYNLSRNSDNIVSDTNTLT |
| | | | | | AGVTLKQELFEQNSWINLDIAEI |
| | | | | | TARQADAVYAAEQQGLILRVS |
| | | | | | QAYFNVLRAIDDLIFVRAEKAA |
| | | | | | VGRQLEQTKQRFEVGLSAITDV |
| | | | | | HDAQAQYDAVLAQEILSENEVS |
| | | | | | NSYEALREITGTRHSDLYILNTK |
| | | | | | TFSATRPQQTVEELVKRAEEGN |
| | | | | | LNLLAQRISRDAAKERISLAESG |
| | | | | | HLPSLTLDVGYGYTDQENKTDS |
| | | | | | SRDINNNELAGGINFNMPLYSG |
| | | | | | GSISAGVKEAQFRYVAASEQLE |
| | | | | | GTYRTTVKDVRAFYNNINASIG |
| | | | | | ALRAYEQTVISQRSALEATEAG |
| | | | | | FEVGTRTIVDVLDATRRLYDAN |
| | | | | | RNLSNARYDYILNVLQLRQAIG |
| | | | | | TLSEQDLVDINNGLLPPKK |
| 1 | WP_025802511.1 | outer | Hafnia alvei | 383 | MKKLLPLLIGLSLGGFTTLSHAE |
| | | membrane | | | NLLQVYQQAKESNPDLRKAAA |
| | | channel | | | DRDVAFEKINESRSPLLPQLGLG |
| | | protein TolC | | | ADYTYTNGMRDNDGLNSNNY |
| | | | | | GASLQLTQTIFDMSKWRQLSLT |
| | | | | | EKQAGIQDVSYQSSEQTLMLNS |
| | | | | | ATAYFNVLRAIDALSYIEANKE |
| | | | | | AIYRQLDQTTQRFNVGLVAITD |
| | | | | | VQNARAQYDLVLANEVTARND |
| | | | | | LDNSLEALRQITGQYYGQLDSL |
| | | | | | NVDRFTTTKPDNVNSLLQEAEK |
| | | | | | RNLSLLSARLSQDLAREQIKYA |
| | | | | | QTGYMPTLDLTASSGVSNTDY |
| | | | | | NSLSNSQKAQNQNGASSYQGQ |
| | | | | | NTIGLQFNLPLYSGGATNSQVQ |
| | | | | | QAQYNFVGASESLESAHRSVVQ |
| | | | | | NVRSSFNNISASISGVEAYRQAV |
| | | | | | ISAQSSLEATEAGYQVGTRTIVD |
| | | | | | VLNATTTLYSAKESLSNARYDY |
| | | | | | LISQLNIKYALGTLNQNDLMAL |
| | | | | | NGGLTKPVSTTPDIIAPQGKTQA |
| | | | | | AQVNSDYNTTASAPVAQPASAS |
| | | | | | TTTSSSSKGKNPFRH |
| 1 | WP_061059312.1 | outer | Hafnia paralvei | 384 | MKKLLPLLIGLSLGGFTTLSHAE |
| | | membrane | | | NLMQVYQQAKESNPDLRKAAA |
| | | channel | | | DRDVAFEKINESRSPLLPQLGLG |
| | | protein TolC | | | ADYTYTNGMRDNDGLNSNNY |
| | | | | | GASLQLTQTIFDMSKWRQLSLT |
| | | | | | EKQAGIQDVSYQSSEQTLMLNS |
| | | | | | ATAYFNVLRAIDALSYIEANKE |
| | | | | | AIYRQLDQTTQRFNVGLVAITD |
| | | | | | VQNARAQYDLVLANEVTARNN |
| | | | | | LDNSLEALRQITGQYYGQLDSL |
| | | | | | NVERFSTSKPENVNSLLQEAEK |
| | | | | | RNLSLLSARLSQDLAREQIKYA |
| | | | | | QTGYMPTLDLTASSGVSNTDY |
| | | | | | NSLSNSQKAQNSNGASSYQGQ |
| | | | | | NTIGLQFNLPLYSGGATNSQVQ |
| | | | | | QAQYNFVGASESLESAHRSVIQ |
| | | | | | NVRSSFNNISASISGVEAYRQAV |
| | | | | | VSAQSSLEATEAGYQVGTRTIV |
| | | | | | DVLNATTTLYSAKESLSNARYD |
| | | | | | YLISQLNIKYALGTLNQNDLMA |
| | | | | | LNGGLTKPVSTTPDIIAPQGKTQ |
| | | | | | ADQVNSDYSAAPVAQATPVSST |
| | | | | | SSSKGKNPFRN |
| 1 | WP_009287284.1 | MULTISPECIES: | Halomonas | 385 | MRLAHWAVNAPLRPLLLAVFT |
| | | TolC | | | ASFVLPAQAADLITITRDALDN |
| | | family outer | | | NAELASARSEYLGVEAGRDVA |
| | | membrane | | | RGGLLPQINASGTVAHNEQYES |
| | | protein | | | QGSSRAGTGAGTGNVSAGDDR |
| | | | | | YNTVALTLEATQALYDEVTRR |
| | | | | | QVTQAERQIDQQVYLLAATEQ |
| | | | | | QLLIDVASAYFDILRAYEVLEA |
| | | | | | RFAQERAIGRQLEQAGEQFEVG |
| | | | | | LIAITEVEEARATFDQSRADRIA |
| | | | | | AESNLQVAFEALEQLTGQRYAS |
| | | | | | IEALGDSMPIALPEPSNRDYWV |
| | | | | | EQAIERNPQVLAQQAGIEVSRS |
| | | | | | GVEIARAGRLPTLSAFGNYQYG |
| | | | | | DSDIDNTTGNDSSSQVGIRANLP |
| | | | | | VYTGGSTSASIRQGTYQLESSQ |
| | | | | | YDFESQRRSSIQQVRSLYTQVS |
| | | | | | NDVETVEAREQAIVSNQSALEA |
| | | | | | TRAGYEVGTRNIVDVLNAEQN |
| | | | | | LYNAIANYAEARYDYVVNLLS |
| | | | | | LRQQAGRLDVDAIEEVNAWLT |
| | | | | | GDEVNFTLPESGGNDRYESALN |
| | | | | | IGAPPQPGT |
| 1 | WP_046079959.1 | MULTISPECIES: | Halomonas | 386 | MKPYRPVSSGRRVLLPCLLAVS |
| | | TolC | | | LAGQAQAADLLTITRDAIDNNA |
| | | family outer | | | SLASARAQYGSVEAGRDVARG |
| | | membrane | | | DLLPQVSASGQVDHNRLYESVS |
| | | protein | | | SSRGSGAGNGDDSYNSASLTLE |
| | | | | | ATQALFDAANSAEVDQAASRID |
| | | | | | QQTYQLAATEQQVLIDVASAYF |
| | | | | | DILRAHEILEARRAQERAIGRQL |
| | | | | | EQAQEQFDVGLIAITEVEEARAS |
| | | | | | FDQSRADRIAAENDLQVSFETL |
| | | | | | ERLTGQRYDSIDALQEAMPVTP |
| | | | | | PQPSGRDAWVELAMDNSPLVL |
| | | | | | ASQAGVEVSEDAVDIARAQRLP |
| | | | | | VVEAFANYSYSDTDQDQMDGH |
| | | | | | NSQSQVGARVSLPIYTGGRTSA |
| | | | | | GIRQSTYDLESSQYDFEDQRRQ |
| | | | | | TVQQVRSLYTQVINDVATVEA |
| | | | | | RAQAIVSNQSALDATRAGYEV |
| | | | | | GTRNIVDVLNAEQSLYDAIADH |
| | | | | | ADARFSYVIDLLTLRQQSGTLN |
| | | | | | VQAIEAINDWLSAEQGVSLALP |
| | | | | | EVGEASRYDEAMNIGAPPRPES |
| 1 | WP_109637673.1 | TolC family | Halomonas | 387 | MPSRPVTPPSRVARRALLPMLV |
| | | outer | elongata | | ASLMAGQVQAADLLTITRDAL |
| | | membrane | | | DNNADLAAARSDTDSVEAGRD |
| | | protein | | | VERGDLLPQVNASGQVAHNEQ |
| | | | | | FESQSSAFSAGSTPSDDRYNSAS |
| | | | | | LTLEASQVLYDAVNSAELEQAD |
| | | | | | RQIDQQTYQLAATEQQVLIDVA |
| | | | | | SAYFDILRAHEILEARRAQERAI |
| | | | | | GRQLEQAREQFDVGLIAVTEVE |
| | | | | | EAQASYDQSRADRISAESDLQV |
| | | | | | AFEALERLTGKRYDSIESLEDA |
| | | | | | MPVTPPEPSKRDAWIDLAMEN |
| | | | | | NPLVLAQQAGVEVSRSGVDVS |
| | | | | | KAQRLPVVNAFANYQYADSDN |
| | | | | | DTLEGEDSSAQVGVKLSVPIYT |
| | | | | | GGRTSAGIRQSTYALESSQYNF |
| | | | | | EAQRRDTIQQVRSLYTQVINDV |
| | | | | | ATVEARAQAIVSNQSALDATRA |
| | | | | | GYEVGTRNIVDVLNAEQNLYD |
| | | | | | AIANHADARYDYVINLLTLRQQ |
| | | | | | SGTLDVDAIEEVNGWLDENQS |
| | | | | | VSFTLPEEGRYEQALDIGEPPRP |
| | | | | | DA |
| 1 | WP_035559610.1 | TolC family | Halomonas sp. | 388 | MRLAHWAVNAPLRPLLLAVFT |
| | | outer | KO116 | | ASFVLPAQAADLLTITRDALDN |
| | | membrane | | | NAALASARSEYLGVEAGRDVA |
| | | protein | | | RSGLLPQVNATGNVAHNQQYE |
| | | | | | SKTSSRAGTGTGTGNVSTGDDR |
| | | | | | YNTVSLTLEATQALYNEVTRRE |
| | | | | | VTQAERQIDQQVYLLAATEQQL |
| | | | | | LIDTASAYFDILRAHEILEARFA |
| | | | | | QERAIGRQLEQAGEQFEVGLIAI |
| | | | | | TEVEEARATFDQSRADRIAAES |
| | | | | | NLQVAFEALEQLTGQRYASIEA |
| | | | | | LGDSMPVGLPEPSSRDYWVEQ |
| | | | | | ALELNPQVLAQQAGIEVARSGV |
| | | | | | EIARAGNLPTLQAFGNYQYSDS |
| | | | | | DVDNTTGEDSSSQVGLSANLPI |
| | | | | | YTGGRTSASIRQGTYQLESSQY |
| | | | | | DFESQRRSSIQQVRSLYTQVSN |
| | | | | | DVETVEARQQAIVSNQSALEAT |
| | | | | | RAGYEVGTRNIVDVLNAEQNL |
| | | | | | YNAIANYAEARYDYVVNLLSL |
| | | | | | RQQAGRLDVEAIEEVNAWLTG |
| | | | | | DEVNFILPESDGNDRYDSALDIG |
| | | | | | APPQPGM |
| 1 | WP_149285557.1 | TolC family | Halomonas sp. | 389 | MTPSRLDSPAARPNMSRRLLGA |
| | | outer | Y2R2 | | MVAALLAGQVQAADLLTITRD |
| | | membrane | | | ALDNNSDLASARATTSGTEENR |
| | | protein | | | NVARADLLPQLTASGEVAYNS |
| | | | | | VFEQQSQKGFSGTVGDDRYTSY |
| | | | | | GLNLEATQAIYDARNSREVGVA |
| | | | | | EKQIDQQTYVLAATEQQVLIDT |
| | | | | | ATAYFDILRANDILDARRAQER |
| | | | | | AIGRQLEQVQEQFDVGLVAITD |
| | | | | | VEEARATFDQSRADRISAESDL |
| | | | | | QVAFEALERLTGKRYDSIDKLQ |
| | | | | | DELPIALPTPSSRDAWIDTAIESS |
| | | | | | PDLLAQKSGVDVAREQVSVSK |
| | | | | | ADRLPVVSGFVNYSYADDDQD |
| | | | | | YLEDYNSTGQVGVNASLPLYT |
| | | | | | GGRTSASIRQNTYNLESSQYDF |
| | | | | | ESQRRETIQQVRALYTQVSNDV |
| | | | | | STVAARQQTIVSSQSALDATRA |
| | | | | | GYEVGTRNIVDVLSAEQNLYN |
| | | | | | AIANYADARYSYVTNLLTLRRQ |
| | | | | | AGVLDEAAIETINQYLQSSGQV |
| | | | | | TFTLPENNGGTYDAALDIGERP |
| | | | | | QLEQ |
| 1 | WP_004174363.1 | MULTISPECIES: | Klebsiella | 390 | MKKLLPILIGLSLTGFSAMSQAE |
| | | outer | | | NLLQVYQQARISNPDLRKSAAD |
| | | membrane | | | RDAAFEKINEARSPLLPQLGLG |
| | | channel | | | ADYTYNNGYRDSNGINSNVTS |
| | | protein TolC | | | GSLQLTQVLFDMSKWRALTLQ |
| | | | | | EKTAGIQDVTYQTDQQTLILNT |
| | | | | | ATAYFKVLAAIDTLSYTEAQKQ |
| | | | | | AIYRQLDQTTQRFNVGLVAITD |
| | | | | | VQNARSQYDAVLANEVTARND |
| | | | | | LDNAVEELRQVIGNYYPELASL |
| | | | | | NVNGFKTNKPQAVNALLKEAE |
| | | | | | NRNLSLLQARLNQDLAREQIRQ |
| | | | | | AQDGHLPTLSLSASSGISNTSYS |
| | | | | | GSKTHNNPQQYQDNDAGQNQI |
| | | | | | GLNFSLPLYQGGAVTSQVKQA |
| | | | | | QYNFVGASEQLESAHRSVVQT |
| | | | | | VRSSFNNVNASISSINAYKQAV |
| | | | | | VSAQSSLDAMEAGYSVGTRTIV |
| | | | | | DVLDATTTLYNAKQQLSNARY |
| | | | | | NYLINELNIKSALGTLNEQDLV |
| | | | | | ALNNTLGKPISTSADSVAPENPQ |
| | | | | | QDATADGYGNTTAAVKPASAR |
| | | | | | TTQSSGSNPFRQ |
| 1 | WP_004205242.1 | MULTISPECIES: | Klebsiella | 391 | MKKLLPILIGLSLTGFSAMSQAE |
| | | outer | | | NLLQVYQQARISNPDLRKSAAD |
| | | membrane | | | RDAAFEKINEARSPLLPQLGLG |
| | | channel | | | ADYTYTSGFRDYKDQNSNVTS |
| | | protein TolC | | | GSLQLTQVLFDMSKWRALTLQ |
| | | | | | EKAAGIQDVTYQTDQQTLILNT |
| | | | | | ATAYFKVLAAIDTLSYTEAQKQ |
| | | | | | AIYRQLDQTTQRFNVGLVAITD |
| | | | | | VQNARSQYDAVLANEVTARND |
| | | | | | LDNAVEELRQVIGNYYPELASL |
| | | | | | NVDGFKTSKPQAVNALLKEAE |
| | | | | | NRNLSLLQARLNQDLAREQIRQ |
| | | | | | AQDGHLPTLDLNASSGVSNNR |
| | | | | | YSGSKSISQDADIGQNKIGLSFS |
| | | | | | LPLYQGGMVNSQVKQAQYNFV |
| | | | | | GASEQLESAHRSVVQTVRSSFN |
| | | | | | NVNASISSINAYKQAVVSAQSS |
| | | | | | LDAMEAGYSVGTRTIVDVLDA |
| | | | | | TTTLYNAKQQLSNARYNYLINE |
| | | | | | LNIKSALGTLNEQDLIALNNTLG |
| | | | | | KPISTSADSVAPENPQQDATAD |
| | | | | | GYGNTTAAMKPASARTTTHSS |
| | | | | | GSNPFRQ |
| 1 | WP_004854252.1 | MULTISPECIES: | Klebsiella | 392 | MKKLFPILIGLGLTGFSAMSQAE |
| | | outer | | | NLLQVYQQARVSNPDLRKSAA |
| | | membrane | | | DRDAAFEKINEARSPLLPQLGL |
| | | channel | | | GADYTYTSGFRDAKDQNSDVT |
| | | protein TolC | | | SGSLQLTQVLFDMSKWRALTL |
| | | | | | QEKNAGIQDVTYQTDQQTLILN |
| | | | | | TATAYFKVLAAIDTLSYTEAQK |
| | | | | | QAIYRQLDQTTQRFNVGLVAIT |
| | | | | | DVQNARSQYDAVLANEVTARN |
| | | | | | DLDNAVEGLRQVIGNYYPELA |
| | | | | | SLNVDGFKTTKPSAVNALLKEA |
| | | | | | ENRNLSLLQARLSQDLAREQIR |
| | | | | | QAQDGHLPTLNLNASSSVSNNS |
| | | | | | YSGSKNTTQDRDIGQNQIGLNF |
| | | | | | SLPLYQGGMVNSQVKQAQYNF |
| | | | | | VGASEQLESAHRSVVQTVRSSF |
| | | | | | NNVNASISSINAYKQAVVSAQS |
| | | | | | SLDAMEAGYSVGTRTIVDVLD |
| | | | | | ATTTLYNAKQQLSNARYNYLIN |
| | | | | | ELNIKSALGTLNEQDLVALNNT |
| | | | | | LGKAIPTSPDSVAPENPQQDAS |
| | | | | | ADGYSNTAAAKPASARSTSGSN |
| | | | | | PFRQ |
| 1 | WP_008806517.1 | MULTISPECIES: | Klebsiella | 393 | MKKLLPILIGLSLTGFSAMSQAE |
| | | outer | | | NLLQVYQQARISNPDLRKSAAD |
| | | membrane | | | RDAAFEKINEARSPLLPQLGLG |
| | | channel | | | ADYTYNNGYRDSNGINSNVTS |
| | | protein TolC | | | GSLQLTQVLFDMSKWRALTLQ |
| | | | | | EKTAGIQDVTYQTDQQTLILNT |
| | | | | | ATAYFKVLAAIDTLSYTEAQKQ |
| | | | | | AIYRQLDQTTQRFNVGLVAITD |
| | | | | | VQNARSQYDAVLANEVTARND |
| | | | | | LDNAVEELRQVIGNYYPELASL |
| | | | | | NVNGFKTNKPQAVNALLKEAE |
| | | | | | NRNLSLLQARLNQDLAREQIRQ |
| | | | | | AQDGHLPTLSLSASSGVSNTSY |
| | | | | | SGSKTHNNPQQYQDNDAGQNQ |
| | | | | | IGLNFSLPLYQGGAVTSQVKQA |
| | | | | | QYNFVGASEQLESAHRSVVQT |
| | | | | | VRSSFNNVNASISSINAYKQAV |
| | | | | | VSAQSSLDAMEAGYSVGTRTIV |
| | | | | | DVLDATTTLYNAKQQLSNARY |
| | | | | | NYLINELNIKSALGTLNEQDLV |
| | | | | | ALNNTLGKPIPTSADSVAPENPQ |
| | | | | | QDATADGYGNTTAAVKPASAR |
| | | | | | TTQSSGSNPFRQ |
| 1 | WP_009308670.1 | MULTISPECIES: | Klebsiella | 394 | MKKLLPILIGLSLTGFSAMSQAE |
| | | outer | | | NLLQVYQQARISNPDLRKSAAD |
| | | membrane | | | RDAAFEKINEARSPLLPQLGLG |
| | | channel | | | ADYTYTNGYRDSNGVNSNVTS |
| | | protein TolC | | | GSLQLTQVLFDMSKWRALTLQ |
| | | | | | EKTAGIQDVTYQTDQQTLILNT |
| | | | | | ATAYFKVLAAIDTLSYTEAQKQ |
| | | | | | AIYRQLDQTTQRFNVGLVAITD |
| | | | | | VQNARSQYDAVLANEVTARND |
| | | | | | LDNAVEGLRQVIGNYYPELAS |
| | | | | | LNVNGFKTNKPQAVNALLKEA |
| | | | | | ENRNLSLLQARLSQDLAREQIR |
| | | | | | QAQDGHLPTLNLSASTGVSNTR |
| | | | | | YNGSKTNTPLAYNDSDNGQNQI |
| | | | | | GLNFSLPLYQGGAVTSQVKQA |
| | | | | | QYNFVGASEQLESAHRSVVQT |
| | | | | | VRSSFNNVNASISSINAYKQAV |
| | | | | | VSAQSSLDAMEAGYSVGTRTIV |
| | | | | | DVLDATTTLYNAKQQLSNARY |
| | | | | | NYLINELNIKSALGTLNEQDLV |
| | | | | | ALNNTLGKPISTSADSVAPENPQ |
| | | | | | QDATADGYGNTTAAVKPVSAR |
| | | | | | TTQSSGSNPFRQ |
| 1 | WP_020316753.1 | MULTISPECIES: | Klebsiella | 395 | MKKLLPILIGLSLTGFSAMSQAE |
| | | outer | | | NLLQVYQQARISNPDLRKSAAD |
| | | membrane | | | RDAAFEKINEARSPLLPQLGLG |
| | | channel | | | ADYTYNNGYRDSNGINSNVTS |
| | | protein TolC | | | GSLQLTQVLFDMSKWRALTLQ |
| | | | | | EKTAGIQDVTYQTDQQTLILNT |
| | | | | | ATAYFKVLAAIDTLSYTEAQKQ |
| | | | | | AIYRQLDQTTQRFNVGLVAITD |
| | | | | | VQNARSQYDAVLANEVTARND |
| | | | | | LDNAVEELRQVIGNYYPELASL |
| | | | | | NVNGFKTNKPQAVNALLKEAE |
| | | | | | NRNLSLLQARLNQDLAREQIRQ |
| | | | | | AQDGHLPTLSLSASSGISNTSYS |
| | | | | | GSKTHNNPQQYQDNDAGQNQI |
| | | | | | GLNFSLPLYQGGAVTSQVKQA |
| | | | | | QYNFVGASEQLESAHRSVVQT |
| | | | | | VRSSFNNVNASISSINAYKQAV |
| | | | | | VSAQSSLDAMEAGYSVGTRTIV |
| | | | | | DVLDATTTLYNAKQQLSNARY |
| | | | | | NYLINELNIKSALGTLNEQDLV |
| | | | | | ALNNTLGKPISTSADSVAPENPQ |
| | | | | | QDATADGYGNTTAAVKPVSAR |
| | | | | | TNQSSGSNPFRQ |
| 1 | WP_023340732.1 | MULTISPECIES: | Klebsiella | 396 | MKKLLPILIGLSLTGFSAMSQAE |
| | | outer | | | NLLQVYQQARISNPDLRKSAAD |
| | | membrane | | | RDAAFEKINEARSPLLPQLGLG |
| | | channel | | | ADYTYTNGYRDSNGVNSNVTS |
| | | protein TolC | | | GSLQLTQVLFDMSKWRALTLQ |
| | | | | | EKTAGIQDVTYQTDQQTLILNT |
| | | | | | ATAYFKVLAAIDTLSYTEAQKQ |
| | | | | | AIYRQLDQTTQRFNVGLVAITD |
| | | | | | VQNARSQYDAVLANEVTARND |
| | | | | | LDNAVEGLRQVIGNYYPELAS |
| | | | | | LNVNGFKTNKPQAVNALLKEA |
| | | | | | ENRNLSLLQARLSQDLAREQIR |
| | | | | | QAQDGHLPTLNLSASTGVSNTR |
| | | | | | YNGSKTNTPLAYNDSDVGQNQI |
| | | | | | GLNFSLPLYQGGAVTSQVKQA |
| | | | | | QYNFVGASEQLESAHRSVVQT |
| | | | | | VRSSFNNVNASISSINAYKQAV |
| | | | | | VSAQSSLDAMEAGYSVGTRTIV |
| | | | | | DVLDATTTLYNAKQQLSNARY |
| | | | | | NYLINELNIKSALGTLNEQDLV |
| | | | | | ALNNTLGKPIPTSADSVAPENPQ |
| | | | | | QDATADGYGNTTAAVKPASAR |
| | | | | | TTQSSGSNPFRQ |
| 1 | WP_049087445.1 | MULTISPECIES: | Klebsiella | 397 | MKKLFPILIGLGLTGFSAMSQAE |
| | | outer | | | NLLQVYQQARISNPDLRKSAAD |
| | | membrane | | | RDAAFEKINEARSPLLPQLGLG |
| | | channel | | | ADYTYTSGFRDYKDQNSNVTS |
| | | protein TolC | | | GSLQLTQVLFDMSKWRALTLQ |
| | | | | | EKNAGIQDVTYQTDQQTLILNT |
| | | | | | ATAYFKVLAAIDTLSFTEAQKQ |
| | | | | | AIYRQLDQTTQRFNVGLVAITD |
| | | | | | VQNARSQYDAVLANEVTARNS |
| | | | | | LDNAVEELRQVIGNYYPELASL |
| | | | | | NVDGFKTTKPSAVNALLKEAE |
| | | | | | NRNLSLLQARLSQDLAREQIRQ |
| | | | | | AQDGHLPTLNLNASSGVSNNR |
| | | | | | YSGSKSLAQDADVGQNKIGLSF |
| | | | | | SLPLYQGGMVNSQVKQAQYNF |
| | | | | | VGASEQLESAHRSVVQTVRSSF |
| | | | | | NNVNASISSINAYKQAVVSAQS |
| | | | | | SLDAMEAGYSVGTRTIVDVLN |
| | | | | | ATTTLYEAKQQLSNTRYNYLIN |
| | | | | | ELNIKSALGTLNEQDLVALNNT |
| | | | | | LGKPVPTSPDSVAPENPQQDAS |
| | | | | | ADGYSNTAAAKPASARSTSGSN |
| | | | | | PFRQ |
| 1 | WP_015369648.1 | outer | Klebsiella | 398 | MKKLFPILIGLGLTGFSAMSQAE |
| | | membrane | aerogenes | | NLLQVYQQARISNPDLRKSAAD |
| | | channel | | | RDAAFEKINEARSPLLPQLGLG |
| | | protein TolC | | | ADYTYTSGFRDYKDRNSNVTS |
| | | | | | GSLQLTQTLFDMSKWRALTLQ |
| | | | | | EKTAGIQDVTYQTDQQKLILNT |
| | | | | | ATAYFKVLAAIDTLSYTEAQKQ |
| | | | | | AIYRQLDQTTQRFNVGLVAITD |
| | | | | | VQNARSQYDAVLANEVTARND |
| | | | | | LDNAVEELRQVTGNYYPELASL |
| | | | | | NVNGFKTSKPQAVNALLKEAE |
| | | | | | NRNLSLLQARLNQDLAREQIRQ |
| | | | | | AQDGHLPTLDLNASTSVSNNRY |
| | | | | | SGSKNISPDADIGQNTVGLNFTL |
| | | | | | PLYQGGMVNSQVKQAQYNFV |
| | | | | | GASEQLESAHRSVVQTVRSSFN |
| | | | | | NVNASISSINAYKQAVVSAQSS |
| | | | | | LDAMEAGYSVGTRTIVDVLDA |
| | | | | | TTTLYNAKQQLSNARYNYLINE |
| | | | | | LNIKSALGTLNEQDLIALNNTLG |
| | | | | | KAIPTSPDSVAPENPQQDAAAD |
| | | | | | GYANTASAQPAAARTTKTSGS |
| | | | | | NPFSH |
| 1 | WP_112213708.1 | outer | Klebsiella | 399 | MKKLFPILIGLGLTGFSAMSQAE |
| | | membrane | huaxiensis | | NLMQVYQQARISNPDLRKSAA |
| | | channel | | | DRDAAFEKINEARSPLLPQLGL |
| | | protein TolC | | | GADYTYTSGFRDSKDQNSNVTS |
| | | | | | GALQLTQVLFDMSKWHALTLQ |
| | | | | | EKTAGIQDVTYQTDQQTLILNT |
| | | | | | ATAYFKVLAAIDTLSYTEAQKQ |
| | | | | | AIYRQLDQTTQRFNVGLVAITD |
| | | | | | VQNARSQYDAVLANEVTARNE |
| | | | | | LDNAVEELRQVIGNYYPELASL |
| | | | | | NVDGFKTNKPSAVNALLKEAE |
| | | | | | NRNLSLLQARLSQDLAREQIRQ |
| | | | | | AQDGHMPTLNLNASSSVSNSSY |
| | | | | | SGSKSTTPDRDIGQNQIGLSFNL |
| | | | | | PLYQGGMVNSQVKQAQYNFV |
| | | | | | GASEQLESAHRSVVQTVRSSFN |
| | | | | | NVNASISSINAYKQAVVSAQSS |
| | | | | | LDAMEAGYSVGTRTIVDVLDA |
| | | | | | TTTLYNAKQQLSNARYNYLINQ |
| | | | | | LNIKSALGTLNEQDLVALNNTL |
| | | | | | GKPIPTSPDSVAPENPQQDASAD |
| | | | | | GYSNTAAAKPASARTTSTSGSN |
| | | | | | PFRQ |
| 1 | WP_032433534.1 | outer | Klebsiella | 400 | MKKLLPILIGLSLTGFSAMSQAE |
| | | membrane | pneumoniae | | NLLQVYQQARISNPDLRKSAAD |
| | | channel | | | RDAAFEKINEARSPLLPQLGLG |
| | | protein TolC | | | ADYTYNNGYRDSNGINSNVTS |
| | | | | | GSLQLTQVLFDMSKWRALTLQ |
| | | | | | EKTAGIQDVTYQTDQQTLILNT |
| | | | | | ATAYFKVLAAIDTLSYTEAQKQ |
| | | | | | AIYRQLDQTTQRFNVGLVAITD |
| | | | | | VQNARSQYDAVLANEVTARND |
| | | | | | LDNAVEELRQVIGNYYPELASL |
| | | | | | NVNGFKTNKPQAVNALLKEAE |
| | | | | | NRNLSLLQARLNQDLAREQIRQ |
| | | | | | AQDGHLPTLSLSASSGISNTSYS |
| | | | | | GSKTHNNPQQYQDNDAGQNQI |
| | | | | | GLNFSLPLYQGGAVTSQVKQA |
| | | | | | QYNFVGASEQLESAHRSVVQT |
| | | | | | VRSSFNNVNASISSINAYKQAV |
| | | | | | VSAQSSLDAMEAGYSVGTRTIV |
| | | | | | DVLDATTTLYNAKQQLSNARY |
| | | | | | NYLINELNIKSALGTLNEQDLV |
| | | | | | ALNNTLGKPISTSADSVAPENPQ |
| | | | | | QDATADGYGNTTAAVKPASAR |
| | | | | | TNQSSGSNPFRQ |
| 1 | YP_005228876.1 | outer | Klebsiella | 401 | MKKLLPILIGLSLTGFSAMSQAE |
| | | membrane | pneumoniae | | NLLQVYQQARISNPDLRKSAAD |
| | | channel | subsp. | | RDAAFEKINEARSPLLPQLGLG |
| | | protein | pneumoniae | | ADYTYTNGYRDSNGVNSNVTS |
| | | | HS11286 | | GSLQLTQVLFDMSKWRALTLQ |
| | | | | | EKTAGIQDVTYQTDQQTLILNT |
| | | | | | ATAYFKVLAAIDTLSYTEAQKQ |
| | | | | | AIYRQLDQTTQRFNVGLVAITD |
| | | | | | VQNARSQYDAVLANEVTARND |
| | | | | | LDNAVEGLRQVIGNYYPELAS |
| | | | | | LNVNGFKTNKPQAVNALLKEA |
| | | | | | ENRNLSLLQARLSQDLAREQIR |
| | | | | | QAQDGHLPTLNLSASTGVSNTR |
| | | | | | YNGSKTNTPLAYNDSDNGQNQI |
| | | | | | GLNFSLPLYQGGAVTSQVKQA |
| | | | | | QYNFVGASEQLESAHRSVVQT |
| | | | | | VRSSFNNVNASISSINAYKQAV |
| | | | | | VSAQSSLDAMEAGYSVGTRTIV |
| | | | | | DVLDATTTLYNAKQQLSNARY |
| | | | | | NYLINELNIKSALGTLNEQDLV |
| | | | | | ALNNTLGKPISTSADSVAPENPQ |
| | | | | | QDATADGYGNTTAAVKPASAR |
| | | | | | TTQSSGSNPFRQ |
| 1 | WP_007370086.1 | MULTISPECIES: | Kosakonia | 402 | MKKLLSLLIGLSLTGFSTMSQA |
| | | outer | | | ENLLQVYQQARLSNPDLRKSA |
| | | membrane | | | ADRDAAFEKINEARSPLLPQLG |
| | | channel | | | LGADYTYTNGFRDSNGVDSNV |
| | | protein TolC | | | TSGSLQLTQTIFDMSKWRALTL |
| | | | | | QEKTAGIQDVTWQTDQQTLILN |
| | | | | | TATAYFNVLSAIDALSYTEAQK |
| | | | | | QAIYRQLDQTTQRFNVGLVAIT |
| | | | | | DVQNARSQYDSVLANEVTARN |
| | | | | | NLDNALESLRQVIGNYYPELAS |
| | | | | | LNVDGFKTDKPKPVNALLKEA |
| | | | | | ESRNLTLLQARLSQDLAREQIR |
| | | | | | QAQDGHLPTLNLTASTSVSNTK |
| | | | | | YSGSATRGNAVSSSSYADRDIG |
| | | | | | QNSVGLSFSLPLYSGGSVTSQV |
| | | | | | KQAQYNFVGASEQLESAHRSV |
| | | | | | VQNVRSSFNNVNASISSINAYK |
| | | | | | QAVVSAQSSLDAMEAGYSVGT |
| | | | | | RTIVDVLDATTTLYNAKQQLSS |
| | | | | | ARYNYLINELNIKSALGTLNEQ |
| | | | | | DLQALNNSLGKNVSTTPESVAP |
| | | | | | ENPQQDASVDNFNGNVQPAAA |
| | | | | | RTSVKR |
| 1 | WP_064563544.1 | outer | Kosakonia oryzae | 403 | MKKLLSLLIGLSLTGFSTMSQA |
| | | membrane | | | ENLLQVYQQARLSNPDLRKSA |
| | | channel | | | ADRDAAFEKINEARSPLLPQLG |
| | | protein TolC | | | LGADYTYTNGFRDSNGVDSNV |
| | | | | | TSGSLQLTQTIFDMSKWRALTL |
| | | | | | QEKTAGIQDVTWQTDQQTLILN |
| | | | | | TATAYFNVLSAIDALSYTEAQK |
| | | | | | QAIYRQLDQTTQRFNVGLVAIT |
| | | | | | DVQNARSQYDSVLANEVTARN |
| | | | | | NLDNALESLRQITGNYYPELAS |
| | | | | | LNVDGFKTDKPKPVNALLKEA |
| | | | | | ESRNLTLLQARLSQDLAREQIR |
| | | | | | QAQDGHLPTLNLTASTSVSNTK |
| | | | | | YSGSATRGNPVASSSYADRDIG |
| | | | | | QNSVGLSFSLPLYSGGSVTSQV |
| | | | | | KQAQYNFVGASEQLESAHRSV |
| | | | | | VQNVRSSFNNVNASISSINAYK |
| | | | | | QAVVSAQSSLDAMEAGYSVGT |
| | | | | | RTIVDVLDATTTLYNAKQQLSS |
| | | | | | ARYNYLINELNIKSALGTLNEQ |
| | | | | | DLQALNNSLGKNVSTAPESVAP |
| | | | | | ENPQQDASVDNFRGNVQPAAA |
| | | | | | RTSVKR |
| 1 | WP_135842909.1 | TolC family | Legionella | 404 | MLSVVGLSMPLHAADLMDVY |
| | | outer | geestiana | | QQALESDPAFKQAYDTYLSTSE |
| | | membrane | | | AIPQAISALRPQVAVGAQAGRS |
| | | protein | | | YEDASVGATAFDTTYFNNQWQ |
| | | | | | INASQAVFNFQAWSQVLQARA |
| | | | | | AVKAAQATENDAAQSLILRTAS |
| | | | | | AYLDILLARDTLSFAEAKKRAN |
| | | | | | KRQLDQAQQRFKVGLDAITSV |
| | | | | | YEAQAAYDQSVAEVISARNNQI |
| | | | | | NQNENLRKLTNHVYEFLAPLRD |
| | | | | | SRIPLIRPEPDNVDNWIDTGIRQ |
| | | | | | NYKLYSAKYSLEAARDNIRVQS |
| | | | | | GTGWPTLAIQGNAQQVHNQTT |
| | | | | | QSSFFVPSRATSANIALALNFPM |
| | | | | | FQGGLVASKTRQAQFDFQASSE |
| | | | | | QLEQIYRDVVVNSRIAFNTIIDGI |
| | | | | | SKVKADRQTIISQTNSLESTEAQ |
| | | | | | FQVGTRTMVDVVNAQQRLFDA |
| | | | | | QRQLASDQYNLIRAMLQLKYL |
| | | | | | AGTLNVNDLQEVNSWLDTTRV |
| | | | | | NGFPPAKPTKP |
| 1 | WP_012978890.1 | TolC family | Legionella | 405 | MKKFLFCTLMTLGLLQQAFAT |
| | | outer | longbeachae | | DLMDIYHQALENDPLFKNAYD |
| | | membrane | | | TYMANAEVLPQARSALRPQVGI |
| | | protein | | | TGQASRNFFHVNDGFFTIQDTY |
| | | | | | YNSRTWQISASQAIFNFQAWAK |
| | | | | | VQQAKASVKAAQAVENDAAQ |
| | | | | | DLILRTAKAYFDALYAQDTLNF |
| | | | | | AEAKKRANKRQYDQAQQRFQ |
| | | | | | VGLDPITSVYEAKAAYDQSVAT |
| | | | | | VIASRNNLINQNENLRKLTNHV |
| | | | | | YDSMAPLRNGEIPLIKPEPNYAD |
| | | | | | QWVDTGLKQNYKLLSAKYNLE |
| | | | | | VARENVKALSAGNWPVISLQG |
| | | | | | NTAETSNSLNTTTDNNPLLPVN |
| | | | | | QKQSTVGLALNFPVYQGGLVR |
| | | | | | SQTRQAKHNFQAASEQVEQAY |
| | | | | | RNVVVNSRIAFNTIIDGISKVKA |
| | | | | | DRQSVISQKNSLDSTEAQFEIGT |
| | | | | | RTMVDVVNAQQRLFEAQNLLA |
| | | | | | NDQYNLINAVLTLKYQAGTLN |
| | | | | | ANDLELINSWLQTTRVKGFPQT |
| | | | | | GNTK |
| 1 | WP_010946436.1 | TolC family | Legionella | 406 | MRKSLFCWILTLGVSTHVFATD |
| | | outer | pneumophila | | LMDIYQQALENDTIFKEAYDTY |
| | | membrane | | | MSSTEAIPQARAALYPQVGLGS |
| | | protein | | | QAGRNYQDAVAGAFSANQYY |
| | | | | | GSYLWQVNASQALFNYQAWA |
| | | | | | KVAQAKASVKAAQATENDAA |
| | | | | | QNLILRTAKAYFDVLFAKDTLD |
| | | | | | FAEAKKRANKRQYDQATQRFQ |
| | | | | | VGLDAITSVYEAKAAYDQSIAT |
| | | | | | VIAARNNQINQSENLRKLTNHV |
| | | | | | YETLAPLKDSKIPLVKPEPNDV |
| | | | | | NQWIDTGLKQNYKLYAAKYNL |
| | | | | | EVAKDNVKAISAGNWPVFSLQS |
| | | | | | NASQVHNNASGNTVFIPSKQTQ |
| | | | | | ANIAIAMNFPVFQGGLVQAQTR |
| | | | | | QAQYGFQSTSEKLEQTYRDVIV |
| | | | | | NSRIAFNTITDGISKVKADRQTV |
| | | | | | ISVQNSLQSTEAQFEVGTRTMV |
| | | | | | DVVNAQQRLFEAQEQLARDQY |
| | | | | | DLINSILTLKYLAGTLNVNDLEQ |
| | | | | | INSWLATTRVNGFSPVDNKTSK |
| 1 | WP_027220262.1 | TolC family | Legionella | 407 | MRKSLFCWILTLGVSTHVFATD |
| | | outer | pneumophila | | LMDIYQQALENDTIFKEAYDTY |
| | | membrane | | | MSSTEAIPQARAALYPQVGLGS |
| | | protein | | | QAGRNYQDAVAGAFSANQYY |
| | | | | | GSYLWQVNASQALFNYQAWA |
| | | | | | KVAQAKASVKAAQATENDAA |
| | | | | | QNLILRTAKAYFDVLFAKDTLD |
| | | | | | FAEAKKRANKRQYDQATQRFQ |
| | | | | | VGLDAITSVYEAKAAYDQSIAT |
| | | | | | VIAARNNQINQSENLRKLTNHV |
| | | | | | YETLAPLKDSKIPLVKPEPNDV |
| | | | | | NQWIDTGLKQNYKLYAAKYNL |
| | | | | | EVAKDNVKAISAGNWPVFSLQS |
| | | | | | NASQVHNNASGNTVFIPSKQTQ |
| | | | | | ANIAIAMNFPVFQGGLVQAQTR |
| | | | | | QAQYGFQSTSEKLEQTYRDVV |
| | | | | | VNSRIAFNTITDGISKVKADRQT |
| | | | | | VISVQNSLQSTEAQFEVGTRTM |
| | | | | | VDVVNAQQRLFEAQEQLARDQ |
| | | | | | YDLINSILTLKYLAGTLNVNDLE |
| | | | | | QINSWLATTRVNGFSPVDNKTS |
| | | | | | K |
| 1 | WP_027271022.1 | TolC family | Legionella | 408 | MKKLLFCSLMTLGLLPQAFATD |
| | | outer | sainthelensi | | LMDIYHQALENDPLFKNAYDT |
| | | membrane | | | YMANAEALPQARSALLPQVGIT |
| | | protein | | | GQATRNFFHVNDGFFTIQDTYY |
| | | | | | NSRTWQVSASQAIFNFQAWSK |
| | | | | | VQQAKASVKAAQAIFNNAAQD |
| | | | | | LILRTAKAYFDSLYAQDTLNFA |
| | | | | | EAKKRANKRQYDQAQQRFQV |
| | | | | | GLDPITSVYEAKAAYDQSVATV |
| | | | | | IASRNNLVNQNENLRKLTNHVY |
| | | | | | DSIAPIRDGKIPLIKPEPNYADQ |
| | | | | | WVDTGLKQNYKLLSAKYNLEV |
| | | | | | ARENVKALSAGNWPVISVQGN |
| | | | | | TTETSNSLNTTSDNNPLLPVNQ |
| | | | | | KQSSVGLALNFPVYQGGLVRSQ |
| | | | | | TRQAKHNFQAASEQVEQAYRE |
| | | | | | VVVNSRIAFNTITDGISKVKADR |
| | | | | | QSVISQKNSLDSTEAQFEIGTRT |
| | | | | | MVDVVNAQQRLFEAQNLLAND |
| | | | | | QYNLINAVLTLKYQAGTLNAN |
| | | | | | DLELINSWLETTRVNGFPTTGN |
| | | | | | TK |
| 1 | WP_064327449.1 | outer | Lelliottia | 409 | MKKLLPILIGLSLTGFSAMSQAE |
| | | membrane | amnigena | | NLLQVYQQARLGNPELRKSAA |
| | | channel | | | DRDAAFEKINEARSPLLPQLGL |
| | | protein TolC | | | GADYTYSNGFRDANGVNSNAT |
| | | | | | SASLQLTQTLFDMSKWRALTLQ |
| | | | | | EKSAGIQDVTYQTDQQTLILNT |
| | | | | | ATAYFNVLSAIDSLSYTEAQKQ |
| | | | | | AIYRQLDQTTQRFNVGLVAITD |
| | | | | | VQNARSQYDSVLANEVTARNN |
| | | | | | LDNALESLRQVIGNYYPELASL |
| | | | | | NVDNFKTDKPQAVNALLKEAE |
| | | | | | NRNLALLQARLSQDLAREQIRQ |
| | | | | | AQDGHLPTLGLTASTGVSDTTY |
| | | | | | SGSKTNSAQYDDSNMGQNKVG |
| | | | | | LTFSMPLYQGGMVNSQVKQAQ |
| | | | | | YNFVGASEQLETAHRNVVQTV |
| | | | | | RSSFNNVNASISSINAYKQAVVS |
| | | | | | AQSSLDAMEAGYSVGTRTIVDV |
| | | | | | LDATTTLYNAKQQLSSARYNY |
| | | | | | LINQLNIKSALGTLNEQDLQML |
| | | | | | NNTLGKPVSTAPENVAPENPQQ |
| | | | | | DAAVDNFNGNGNADMPAAQP |
| | | | | | AAARTTTAAPASSGTNPFRH |
| 1 | WP_108900371.1 | outer | Limnobaculum | 410 | MKKLLPLIIGLSLVGLSTSSYAE |
| | | membrane | parvum | | NLIQVYQQAKSNNPDLRKSESD |
| | | channel | | | RDAAYEKISESRGALLPQLGLN |
| | | protein TolC | | | AGYNINRGYRDNSGSKSDVTN |
| | | | | | GTLQLSQTIFDMSLWRQLNITE |
| | | | | | KQAGVQDVSYQAQQQTLILDT |
| | | | | | ATAYFNVLRSLDILSTTEAQKD |
| | | | | | ALYRQLDQARQRFNVGLVAVT |
| | | | | | DVQNAQAQYDQTLADEVTARN |
| | | | | | NLENSLEVLRQITGMFYPELSSL |
| | | | | | DTARFSTNRPNNVNTLLQEAES |
| | | | | | RNLNLLTARLAQDLSREQVKLQ |
| | | | | | ETGHMPTVAFTASSGLSNNDYR |
| | | | | | GTSTIGPEDSINGNTTAGFTLSFP |
| | | | | | LFSGGQTSSRVKQAQYNYIGAS |
| | | | | | EALESAHRSVVQIVRSSYNNVS |
| | | | | | ASISSVNAYKQTVVSAQSSLEA |
| | | | | | TQAGYDVGTRTIVDVLNATTTL |
| | | | | | YNAKQQLSNARYDYLINQLNIK |
| | | | | | YALGTLNENDLDLLNKTLGKP |
| | | | | | NSTIPVINVKE |
| 1 | WP_141065604.1 | TolC family | Luteimonas | 411 | MLRRPLALALSLVLLPASAFAQ |
| | | outer | yindakuii | | DLVQTYELARAGDPQLSIAESN |
| | | membrane | | | REISREGAVQARAALLPQINGS |
| | | protein | | | ADLSRRYVAGVDGDQRSRSVG |
| | | | | | ASLDQTVFDLGRISNLRAQRQL |
| | | | | | DTAAGFDLQAANQTLITRTSAA |
| | | | | | YFNVLVAIETLAAAEAAEQALQ |
| | | | | | KQFDFASRRLEVGLAPITDVHE |
| | | | | | ARAQYDAARAQVILSRNALED |
| | | | | | AYQALAEITGVPVRNLRALPED |
| | | | | | FQPEVPAGGDADSWVNAALGQ |
| | | | | | NPSLRAAEYAVRAAEIGITGAR |
| | | | | | AGHLPRLSLGAGYNNQAYWGD |
| | | | | | LAGVNDPGNPTGARRNEGHSV |
| | | | | | GLTLTVPIFSGGATQSGVREAIA |
| | | | | | RRDIAEDQLTQTRRALERNARN |
| | | | | | AYQTVVAGVSEVEARRLAVLS |
| | | | | | ARSAYDASQVGLEVGTRTVVD |
| | | | | | VLINQQTLFQAEQQFALARYNF |
| | | | | | LQNRLLLEQAVGTLDINDLQEI |
| | | | | | NRLLTVEADSPSTAPTAPASSPQ |
| | | | | | QQ |
| 1 | WP_200608688.1 | TolC family | Lysobacter | 412 | MIRRPLVIALATAMGLSALMPA |
| | | outer | arenosi | | TASAEDLLQTYELARDSDPQLS |
| | | membrane | | | ASESSRLAVKEGAVQTRAAML |
| | | protein | | | PQIDGSASINRSENHNRGNLAV |
| | | | | | GSDGSISGGNSQIDTTTRSSEVR |
| | | | | | LSQMIYDRGNFTRHKSQKALSE |
| | | | | | ASDFQLAAAGNDLITRTSAAYF |
| | | | | | NVLIALESLVAAEAAETALKKQ |
| | | | | | FDYASKRLEVGLAPITDVHEAR |
| | | | | | AQYDSARANTILARNVLEDAY |
| | | | | | QGLTELTGKDVRGVKGLPKDF |
| | | | | | QPVLPESRDAEGWVATAIENNP |
| | | | | | ALRAKDLQVQSTEADVETARA |
| | | | | | GHWPRLYLNGSYGDSTGWGD |
| | | | | | NTLHDLGITRDIDNRSYGPAVG |
| | | | | | VTLSVPIFSGGAVQSGVRQALA |
| | | | | | RRDVASDELESQKRALIRNTRN |
| | | | | | AYQTLVASVSEVEARRLALVSA |
| | | | | | KSAYDASQVGLEVGTRTVLDV |
| | | | | | LQNQNNLFRAQLEYARARYNH |
| | | | | | LQNRLLLEQAAGTLDIADVQDI |
| | | | | | NRLLTLDTESYVAPGDVQG |
| 1 | WP_058090061.1 | TolC family | Marinobacter sp. | 413 | MKKRLLPGLITLLAAQPAFSLD |
| | | outer | LQ44 | | LMETYEKALSYDSGIASAMAQF |
| | | membrane | | | QAQQATSDVSKSALLPKISAVG |
| | | protein | | | SASYTRFKPRNIPGGANADELT |
| | | | | | RQLFAGDSYRTYRYGVELTQPL |
| | | | | | FRAQDWFSYEASQFQTEAAQA |
| | | | | | QYNLAQQQLILDVATAYFNVL |
| | | | | | RAQDTVTTARATEAAIQRQYEQ |
| | | | | | AQERFDVGLIAITEVYEARASY |
| | | | | | DDSKSQRIAAENQLNVAREQLA |
| | | | | | RLTGEYAEDLENLRENFPLGRP |
| | | | | | EPMDPSAWENTALEQNWVIQS |
| | | | | | ALYQLNANEANLKVAKSGHLP |
| | | | | | TLDLFASYGKSELDGAKNTQQE |
| | | | | | GTQGVIGLELNVPLYMGGGTQ |
| | | | | | AGVRQQRSLVTAAQEDLNTTR |
| | | | | | RDVRVNTRSLYLTVNNNVETA |
| | | | | | SALEQTIISRRSALDATRAGYDV |
| | | | | | GTRNIVEVLDAERAYYVALRD |
| | | | | | YANARYDYVINTLQLKQAAGT |
| | | | | | LSPQDLIELNNWLSANARGIEA |
| | | | | | LAEEDLTLDDPTQ |
| 1 | WP_036273387.1 | MULTISPECIES: | Methylomonas | 414 | MTKLLPTFFVALTWGSPAFCQD |
| | | TolC | | | LLETYQLAKANDPEFKSSDINK |
| | | family outer | | | LATAEIKSQSIAQMLPNISFNAN |
| | | membrane | | | SSRNRLESTSFLGTTLQHYWDH |
| | | protein | | | KLGFNLKQPVFHWDHWVQLD |
| | | | | | QADNKIAQAEAQFQAKQQSLIR |
| | | | | | RTAEAYFNILAAQDNLEFADSE |
| | | | | | KKSIEKQLEQAKQRFDVGIIAIT |
| | | | | | DVYEAQAGYDRALASEIEAQN |
| | | | | | QLDNSKEALREIIGENAADLNSL |
| | | | | | QPQIPLSPPAPEDLSSWSNAAEN |
| | | | | | NNFSIVAQLNQAEYVRKNVELQ |
| | | | | | QSKHLPTLDIVAQYGDQDTGNR |
| | | | | | YGLRGDNESVGLQLNLPLFEGG |
| | | | | | GTSSRSRQAAYEYEAAKEDLTK |
| | | | | | VKRSINRGVKDAFRGVVSSISR |
| | | | | | VKALDATSKSAEMAVEAAEAG |
| | | | | | FEVGTRTMVDVLTEQRNLYKA |
| | | | | | KSDYARSRYDYLINGIKLKEAA |
| | | | | | GSLNEQDLEQINQYLQGTLSKN |
| 1 | WP_004237157.1 | MULTISPECIES: | Morganella | 415 | MKKLLSLLIAVSVAGFSSVTQA |
| | | outer | | | EDLLQVYQKAKETNPTLRQTQ |
| | | membrane | | | AERNQAFEKINEARSPLLPQLGL |
| | | channel | | | SGGYDYNTGYRDNSNQSNNAL |
| | | protein TolC | | | NAQLKLTQTIFDMSKWRQLNL |
| | | | | | QEQTAGISDITFQSAQQQLIVDT |
| | | | | | AVAYFNVLRALDTLSFVQAQK |
| | | | | | EAVYRQLDQTTQRFNVGLVAIT |
| | | | | | DVQNARANYDSVLAQEVAGRN |
| | | | | | DVENAIEALRQVSGIYYSELASL |
| | | | | | DVNRFKTNAPDAIEALLKEAQE |
| | | | | | RNLNLLGATLSRNLAKEQISLA |
| | | | | | ESGHYPTVNLSAGTGVTNTDYS |
| | | | | | GNGYPNGRPNNSYAGQSTIGVN |
| | | | | | VSIPLYTGGAVSSQVDQAQYGY |
| | | | | | TAASEKLEATLRQVVQTTRSSY |
| | | | | | NNINASISSVNAYKQVVTSAESS |
| | | | | | LDATEAGYQVGTRTIVDVLNAT |
| | | | | | TTLYEAKKSLSNARYDYLINQL |
| | | | | | YIGQARGTLNEDDILRLNQVLG |
| | | | | | KTISTSPASVAPTTR |
| 1 | WP_112716816.1 | TolC family | Moritella | 416 | MKFNKILLACGLAFFAHSASAD |
| | | outer | yayanosii | | NLQQIFQQALTKDPLYLEAQAN |
| | | membrane | | | RDAALEKITEQEAANLPQIGLSA |
| | | protein | | | DLGYGITSDYTTANTTSNGNAL |
| | | | | | TGSVGIGLTQSLYEESNFINVSQ |
| | | | | | AAKQAEQSELAVQAELQNLILR |
| | | | | | VANAYFSVLGANDTLEFSNRN |
| | | | | | KDAVERQLEQTTQRYNVGLTD |
| | | | | | KTDVLEAQSSFDLSVAEVINAE |
| | | | | | NTLANSYESLTEITGLTHINIATL |
| | | | | | NTARFSPTAPDGQRDNWLATA |
| | | | | | NNQNLAIQIQRIAKQIAEGNVEL |
| | | | | | ASSADNMSLNLVANAGAQYTG |
| | | | | | YGDDYNNGGAGLESTVSSANV |
| | | | | | GIEFSMPLYTGGAVTSAKKQAA |
| | | | | | YQVTASQEQLTRASREVQTKIR |
| | | | | | TFYNNVTAGLSSIKAYEQTVKS |
| | | | | | SQSALEATQAGFGVGTRTIIDVL |
| | | | | | DATKTLYQSKQSLSASRYNYIIS |
| | | | | | MLQLKQAAGTLNAEDIDLVNA |
| | | | | | GLETSN |
| 1 | WP_014605030.1 | MULTISPECIES: | Pantoea | 417 | MKKLLPLFIGLSLGGFSVASQA |
| | | outer | | | ENLLQVYQQARLSNPDLRSSAA |
| | | membrane | | | TRDAAFEKINEARSPLLPQLGLG |
| | | channel | | | ADYSYANGYRDSSGIHSNTTSG |
| | | protein TolC | | | TLQLTQTIFDMSKWRALTLQEK |
| | | | | | TAGIQDVTYQVAQQDLILNTAT |
| | | | | | AYFNVLKAIDTLSYVEAQKMSI |
| | | | | | YRELDQTTQRFNVGLVAITDVQ |
| | | | | | NARAQYDSVLANEVTARNNLD |
| | | | | | NSVESLRQITGMDYLALASLNI |
| | | | | | DRFKTTKPDDVNALLKQAESR |
| | | | | | NLNLLSARLNQDLAREQIRSAE |
| | | | | | SGHLPTLDLTASTGLSNSKYGG |
| | | | | | SRAEQSANSTDSITGNSKVGLSF |
| | | | | | TLPLYSGGSVTSQVKQAQYNFV |
| | | | | | SASEQLEAAHRSAVQTVRSSYN |
| | | | | | NVNASASSIAAYKQAVVSAQSS |
| | | | | | LDAMEAGYQVGTRTIVDVLNA |
| | | | | | TSTLYNAKQQLSDARYSYLINQ |
| | | | | | LNISYALGTLNEQSLGILNSQLG |
| | | | | | KEISTSPETVQPQNVQQVLGED |
| | | | | | HIASSSETATAPVRPAAHKSSRN |
| | | | | | PFAN |
| 1 | WP_024471050.1 | MULTISPECIES: | Pantoea | 418 | MKKLLPLFIGLSLGGFSVASQA |
| | | outer | | | ENLLQVYQQARLSNPDLRSSAA |
| | | membrane | | | TRDAAFEKINEARSPLLPQLGLG |
| | | channel | | | ADYSYANGYRDSSGIHSNTTSG |
| | | protein TolC | | | TLQLTQTIFDMSKWRALTLQEK |
| | | | | | TAGIQDVTYQVAQQDLILNTAT |
| | | | | | AYFNVLKAIDTLSYVEAQKMSI |
| | | | | | YRELDQTTQRFNVGLVAITDVQ |
| | | | | | NARAQYDSVLANEVTARNNLD |
| | | | | | NSVESLRQITGMDYLALASLNI |
| | | | | | DRFKTTKPDNVNALLKQAESR |
| | | | | | NLNLLSARLNQDLAREQIRSAE |
| | | | | | SGHLPTLDLTASTGLSNSKYGG |
| | | | | | SRAEQSANSTDSITGNSKVGLSF |
| | | | | | TLPLYSGGSVTSQVKQAQYNFV |
| | | | | | SASEQLEAAHRSAVQTVRSSYN |
| | | | | | NVNASASSIAAYKQAVVSAQSS |
| | | | | | LDAMEAGYQVGTRTIVDVLNA |
| | | | | | TSTLYNAKQQLSDARYSYLINQ |
| | | | | | LNISYALGTLNEQSLGILNSQLG |
| | | | | | KEISTSPETVQPQNVQQVLGED |
| | | | | | HIASSSETATAPVRPAAHKSSRN |
| | | | | | PFAN |
| 1 | WP_041457811.1 | outer | Pantoea ananatis | 419 | MKKLLPLFIGLSLGGFSVASQA |
| | | membrane | | | ENLLQVYQQARLSNPDLRSSAA |
| | | channel | | | TRDAAFEKINEARSPLLPQLGLG |
| | | protein TolC | | | ADYSYANGYRDSSGIHSNTTSG |
| | | | | | TLQLTQTIFDMSKWRALTLQEK |
| | | | | | TAGIQDVTYQVAQQDLILNTAT |
| | | | | | AYFNVLKAIDTLSYVEAQKMSI |
| | | | | | YRELDQTTQRFNVGLVAITDVQ |
| | | | | | NARAQYDSVLANEVTARNNLD |
| | | | | | NSVESLRQITGMDYLALASLNI |
| | | | | | DRFKTTKPDDVNALLKQAESR |
| | | | | | NLNLLSARLNQDLAREQIRSAE |
| | | | | | SGHLPTLDLTASTGLSNSKYGG |
| | | | | | SRAEQSANSTDSITGNSKVGLSF |
| | | | | | TLPLYSGGSVTSQVKQAQYNFV |
| | | | | | SASEQLEAAHRSAVQTVRSSYN |
| | | | | | NVNASASSIAAYKQAVVSAQSS |
| | | | | | LDAMEAGYQVGTRTIVDVLNA |
| | | | | | TSTLYNAKQQLSDARYSYLINQ |
| | | | | | LNISYALGTLNEQSLGILNSQLG |
| | | | | | KEISTSPETVQPQNVQQVLGED |
| | | | | | HIASSSETATAPVRPSAHKSSRN |
| | | | | | PFAN |
| 1 | WP_013510530.1 | outer | Pantoea sp. At-9b | 420 | MKRLLPLFIGLSLGGFSLASQAE |
| | | membrane | | | NLLQVYQQARLSNPTLRASAA |
| | | channel | | | DRDAAFEKINEARSPLLPQLGL |
| | | protein TolC | | | GADYTYNNGFRDSSGLHSNTTS |
| | | | | | GSLQLTQTIFDMSKWRALTLQE |
| | | | | | KTAGIQDVTYQIAQQDLILNTA |
| | | | | | TAYFNVLNAIDTLSYTEAQKQSI |
| | | | | | YRELDQTTQRFNVGLVAITDVQ |
| | | | | | NARAQYDSVLASEVTARNTLD |
| | | | | | NAVEALRQITGMDYLSLAELNI |
| | | | | | DRFKTTRPDAVSSLLKEAESRN |
| | | | | | LSLLSARLSQDLAREQIRSAETG |
| | | | | | HMPTLDLTASTGLSNSKYGGSR |
| | | | | | ANQSSSTSDSITGSNQVGLSFSL |
| | | | | | PLYSGGSVTSQVKQAQYSFVSA |
| | | | | | SEQLESAHRSAIQTVRSSFNNVN |
| | | | | | ASISSIDAYKQAVVSSQSSLDA |
| | | | | | MEAGYQVGTRTIVDVLDATTT |
| | | | | | LYNAKQQLADARYNYMINQLN |
| | | | | | IKYALGTLNEQDLQLLNAQLGK |
| | | | | | ETSTSPETVAPENTQQASRVDN |
| | | | | | GPKASSASAAAKARPAAARNTS |
| | | | | | NPFSN |
| 1 | WP_061063381.1 | outer | Pantoea vagans | 421 | MKKLLPFFIGLSLGGFSFASQAE |
| | | membrane | | | DLLQVYQQARLSNPDLRSAAA |
| | | channel | | | DRDSAFEKINEARSPLLPQLGLG |
| | | protein TolC | | | ADYTYNNGYRDSSGLHSNTTS |
| | | | | | GTLQLTQTIFDMSKWRALTLQE |
| | | | | | KTAGIQDVTYQVAQQDLILNTA |
| | | | | | TAYFNVLKAIDTLSYTEAQKQSI |
| | | | | | YRELDQTTQRFNVGLVAITDVQ |
| | | | | | NARAQYDSVLANEVTARNNLD |
| | | | | | NNVETLRQITGMDYMSLASLSI |
| | | | | | DRFRTDKPEAVSALLKQAESRN |
| | | | | | LSLLSARLNQDLAREQIRSAETG |
| | | | | | HMPTLDLTASTGMSNSKYGGS |
| | | | | | RANQSTGSTDSITGSNQVGLSFS |
| | | | | | LPLYSGGSVTSQVKQAQYSFVS |
| | | | | | ASEQLESAHRSAVQTVRSSFNN |
| | | | | | VNASISSINAYKQAVVSAQSSL |
| | | | | | DASEAGYQVGTRTIVDVLDATT |
| | | | | | VLYNAKQQLSDARYSYLINQLN |
| | | | | | ISYALGTLNEQSLQKLNSQLGK |
| | | | | | EIPTSPETVAPENAQQTARVDSG |
| | | | | | PVATGSSEAARPAARHSSGNPF |
| | | | | | GN |
| 1 | WP_039282780.1 | MULTISPECIES: | Pectobacterium | 422 | MKKLLPLLIGLSLGGFSAMSQA |
| | | outer | | | ENLLQVYQQAKSTNPDLRSSAA |
| | | membrane | | | TRDAAFEKINESRSPLLPQLGIN |
| | | channel | | | AGYTYNKGYRDSNGVNNNEKS |
| | | protein TolC | | | ATLQLTQTLFDMSKWRALTLQ |
| | | | | | EKQAGIEDVTYQTAQQNLMLN |
| | | | | | TATAYFNVLRAIDSLSYINAQK |
| | | | | | QAIYRQLDQTTQRFNVGLVAIT |
| | | | | | DVQNARAQYDSVLANEVLTRN |
| | | | | | TLDNALESLRQITGNFYPQLAG |
| | | | | | LNIERFSTQKPEAVNNLLKEAE |
| | | | | | NRNLNLLSARLSQDLAREQIRS |
| | | | | | AETGYMPTLDLTASTGVSDTRY |
| | | | | | SGSRTNSGNFNDTDAGQHRVGI |
| | | | | | NFTLPLYSGGATNSQVKQAQHS |
| | | | | | YVSASELLESAHRSVIQTVRSSF |
| | | | | | NNISASISSINAYKQAEVSAQSS |
| | | | | | LDAMEAGYQVGTRTIVDVLDA |
| | | | | | TTTLYNAKQQLSSARYDYLINQ |
| | | | | | LNIKSAQGTLNEADLQALNASL |
| | | | | | GQPVSTTPTVTDNTAPLATTAS |
| | | | | | AQR |
| 1 | WP_012822127.1 | outer | Pectobacterium | 423 | MKKLLPLLIGLSLGGFSAMSQA |
| | | membrane | parmentieri | | ENLLQVYQQAKSTNPDLRSTAA |
| | | channel | | | TRDAAFEKINEARSTLLPQLGIN |
| | | protein TolC | | | AEYTYNKGYRDSNGVNNNEKS |
| | | | | | ASLQLTQTLFDMSRWRALTLQE |
| | | | | | KQAGIEDVVYQTAQQNLMLNT |
| | | | | | ATAYFNVLRAIDSLSYISAQKQ |
| | | | | | AIYRQLDQTTQRFNVGLVAITD |
| | | | | | VQNARAQYDSVLANEVLTRNT |
| | | | | | LDNALESLRQITGNFYPQLAGL |
| | | | | | NITHFSTQKPEAVNNLLKEAEN |
| | | | | | RNLNLLSARLSQDLAREQIRSSE |
| | | | | | TGYMPTLGLTASTGVSDTRYSG |
| | | | | | SRTNSGNFNDTDAGQHRVGINF |
| | | | | | TLPLYNGGITNSQVKQAQHSYV |
| | | | | | SASEQLESAHRSVIQVVRSSFNN |
| | | | | | ISASISSINAYKQAEVSAQSSLD |
| | | | | | AMEAGYQVGTRTIVDVLDATT |
| | | | | | TLYNAKQQLSSARYDYLINQLN |
| | | | | | IKSAQGTLNESDLQALNASLGQ |
| | | | | | PVSTTPTITDDSAPQTTTASAQR |
| 1 | WP_103971350.1 | outer | Pectobacterium | 424 | MKKLLPLLIGLSLGGFSAMSQA |
| | | membrane | versatile | | ENLLQVYQQAKSTNPDLRSSAA |
| | | channel | | | TRDAAFEKINQARSPLLPQLGL |
| | | protein TolC | | | GADYTYNRGYRDSKGVDSNVK |
| | | | | | GASLQLTQTLFDMSKWRALTL |
| | | | | | QEKQAGIEDVTYQTAQQNLML |
| | | | | | NTATAYFNVLRAIDSLSYINAQ |
| | | | | | KQAIYRQLDQTTQRFNVGLVAI |
| | | | | | TDVQNARAQYDSVLANEVLTR |
| | | | | | NTLDNALESLRQITGNFYPQLA |
| | | | | | GLNIERFSTQKPEAVNNLLKEA |
| | | | | | ENRNLNLLSARLSQDLAREQIR |
| | | | | | SAETGYMPTLDLTASTGVSDTR |
| | | | | | YSGSRTQNSNSFNDSDAGQHK |
| | | | | | VGINFTLPLYSGGATNSQVKQA |
| | | | | | QHSYVSSSELLESAHRSVIQTVR |
| | | | | | SSFNNISASISSINAYKQAEVSA |
| | | | | | QSSLDAMEAGYQVGTRTIVDV |
| | | | | | LDATTTLYNAKQQLSSARYDY |
| | | | | | LINQLNIKSAQGTLSETDLQALN |
| | | | | | ASLGQPVSTTPAVTDNSAPLAT |
| | | | | | TASAQR |
| 1 | WP_068945591.1 | outer | Photobacterium | 425 | MKKLLPLFISVAFGGLSQSALA |
| | | membrane | damselae | | DNLAEIYQQAKQNDPQLLKAA |
| | | channel | | | ADKDAAFEAINSARSSLLPQINL |
| | | protein TolC | | | SAGYKGNFMDADTPAVDNHGF |
| | | | | | NTGLQLSQSIYNRSSWVNLDISE |
| | | | | | QQARQSDAAYAAAQQALIMRV |
| | | | | | SKAYFDVLKAQDDLTFVRAEK |
| | | | | | AAVGRQLEQMKQRFDVGLSAI |
| | | | | | TDVYDAQAQYDSVLASEILKEN |
| | | | | | ALTNSYEELREITGQAHKNLDIL |
| | | | | | DTKSFSASNPSKPVNDLVKGAE |
| | | | | | TENLTLLTYRIAQDVARNKISLA |
| | | | | | ESGHLPTLSFDTGYSYDQTRMG |
| | | | | | NDDGSLTAGVTMNLPVYTGGR |
| | | | | | TTSEVKQAQFAYVSASEQLEQQ |
| | | | | | YRSVVKDVRAYYNNINSSIASL |
| | | | | | RAYQQSVVSAKSALEATEAGF |
| | | | | | DVGTRTIVDVLNSTQSLYNANS |
| | | | | | QLADARYNYILSQLQLKQAVG |
| | | | | | TLSEQDILDINQGLKAVAKK |
| 1 | WP_109791821.1 | MULTISPECIES: | Photorhabdus | 426 | MKKLLSIFIAMSLTGLSSMSQAE |
| | | outer | | | DLLQVYKQAKEGNPELRKSMA |
| | | | | | ERNKAFEKINESRSPLLPQLELN |
| | | | | | TGYTYNSGYRDQRDTESNTLN |
| | | | | | AKLGLTQTIFDMSKWRLLNLEE |
| | | | | | KTAGIADVTYQSSEQKLILDSAT |
| | | | | | AYFKVLRTIDTLSYIEAQKAAIY |
| | | | | | RQLDQTTQRFNVGLVAITDVQN |
| | | | | | ARANYDDVLAQEVASRNELDN |
| | | | | | ALEALRQVTGIYYPRLASLNID |
| | | | | | HFKTQKPEAVDILLKEAEKRNL |
| | | | | | SLLSARLGQDLAREQIKAAQTG |
| | | | | | HMPTIDLGASTNVSNSQNHAPG |
| | | | | | RSNNYSGENSVGLTLRLPIYSG |
| | | | | | GATSSRVEQAQYGFVSASEQLE |
| | | | | | STYRNVVQTVRSSFNNISASISSI |
| | | | | | DAYKQLVVSAQSSLDAMEAGY |
| | | | | | QVGTRTIVDVLNATTKLYDAK |
| | | | | | QKLSNARYDYLINQLNIQHALG |
| | | | | | TLNEKDLVALNNSLGKQLSTSA |
| | | | | | NSIVQGTGTPVTSARN |
| 1 | WP_004245559.1 | MULTISPECIES: | Proteus | 427 | MKKLLSLLVTMSLAGFSTASQA |
| | | outer | | | EDLLQVYQKAKDSNPELRKSLA |
| | | membrane | | | ERNQAFEKINEARSPLLPQLGLG |
| | | channel | | | ASIDYKSGYRDAKNTESNSLGA |
| | | protein TolC | | | NLTLTQTIFDMSLWRQLTMQEK |
| | | | | | TAGMSDVTYQTSQQQLILDTAT |
| | | | | | AYFNVLRAIDSLSFIEAQKEQVY |
| | | | | | RQLDQTTQRFNVGLVAITDVQN |
| | | | | | ARANYDSVLAQEVAGRNELDN |
| | | | | | ALEKLRQVSGVYYINLASLNIA |
| | | | | | RFSTTPPDAIDKLLKDAEERNLS |
| | | | | | LLSARLGQDLARENIRLAQSGH |
| | | | | | LPTVNLNASTGVSNSHNHGSAL |
| | | | | | PPETPANSRNSYNGQSSIGLSLSI |
| | | | | | PLYTGGRTSSQVEQAQYGFTSA |
| | | | | | SEQLESVYRSIVQIARSSYNNIS |
| | | | | | ASISSIKAYQQVVVSAQSSLDAT |
| | | | | | EAGYQVGTRTIVDVLNATTTLY |
| | | | | | DAKQKLSSARYDYLINQLNIQY |
| | | | | | ARGTLNENDLIQLNNALGEEIST |
| | | | | | SPDNIIRPLTSPVLNTTN |
| 1 | WP_004925132.1 | MULTISPECIES: | Providencia | 428 | MKKLLPLFIALSFVGLSTNSYAE |
| | | outer | | | DLLQVYQKSKESNPDLRKSLAE |
| | | membrane | | | RNQAFEKINESRSPLLPQLGLGA |
| | | channel | | | GITYGSGYRDTRNTESNGLNAS |
| | | protein TolC | | | LKLTQVVFDMSKWRQLSVQEK |
| | | | | | TAGITDVSYQTSQQQLILDTAT |
| | | | | | AYFNVLKAIDALSYIEANKEAV |
| | | | | | YRQLDQTTQRFNVGLVAITDVQ |
| | | | | | NARANYDSVLAQEVSGRNDLE |
| | | | | | NAVEKLRQVSGVYYNQLASLNI |
| | | | | | DRFKTVTPDQVDAILKEAEERN |
| | | | | | LSLLSARLAQDVSRENIRLAETG |
| | | | | | HMPTINLDAATSVANTHNHGS |
| | | | | | NFNGLDRNSYTGQNSIGLTLSLP |
| | | | | | LYSGGAVSSQVEQAQYGFQGA |
| | | | | | SEQLESVYRNVIQLVRSSYNNV |
| | | | | | SSSISSINAYKQVVVSAQSSLDA |
| | | | | | MEAGYQVGTRTIVDVLTATTA |
| | | | | | LYQAKQNLSNARYDYMINQLN |
| | | | | | IEFARGTLNEEDIARLNASLGKE |
| | | | | | VSTSPSSIIRNIETPQIR |
| 1 | WP_061064623.1 | MULTISPECIES: | Providencia | 429 | MNKIIKLFILISSIIIGYNCNAEDL |
| | | outer | | | LQVYKITKDNNPTLKKTESERY |
| | | membrane | | | QAYKKIDEERSSLLPQLKLKAN |
| | | channel | | | SNYKNNDGTDDNKNIKNTELSL |
| | | protein TolC | | | QMTQSIIDISKWKSLSIQEKHAA |
| | | | | | ISDINYQISQQDLLLETAISYFNI |
| | | | | | LKSIDALTYIKAHKLAVYHQLD |
| | | | | | QTTQRFNVGLTTITDVQNARAN |
| | | | | | YDSIIAQEVLGYNELKNSVEVL |
| | | | | | RKISGVRYTQLAKLNINKLQLT |
| | | | | | KPDNITFLLENARIKNLSLLKAR |
| | | | | | LSQEISNDKISLAKSGHLPIVTLD |
| | | | | | ASTAINNTRLDKNSIESQHKINN |
| | | | | | HSGKNNAALSLNLPIYQGNTVN |
| | | | | | SQVKQAQYGFEAATEQLESDN |
| | | | | | RNVIQQVNSSYNNLLSSISSINA |
| | | | | | YKQLIISTQSSLDATEAGYQVGT |
| | | | | | RTIVDVLSATAALYQSKKNLSN |
| | | | | | AQYDYLINQLKMEYLQGSLNE |
| | | | | | NSIQRINKMLGNEISIYEPTMQ |
| 1 | WP_058429710.1 | MULTISPECIES: | Pseudoalteromonas | 430 | MKKNILAALVSLSCALGTTAAN |
| | | outer | | | AEDLLQVFEIATANDPTVLKAK |
| | | membrane | | | AQADAQSYQADTAMAALLPQI |
| | | channel | | | GLTMGYTKSDSTSFESVDEKVE |
| | | protein TolC | | | SDSNQFTRGLSLSQTLFDLGAW |
| | | | | | NSLDIADKQALQANAQYDSAK |
| | | | | | QNLIVRVAEGYFNVLSAIDNLE |
| | | | | | FIKTEKRAIERQLEQTKQRYAV |
| | | | | | GLTAITDVHEAQAQFDNSVAQE |
| | | | | | IIANNSVETAREQLREITGKYHA |
| | | | | | KLDFLNTETFSTTKPARDSSDFL |
| | | | | | NIAKDKNISLQVAKVTVDIAQD |
| | | | | | QIKLARAGHYPKLTLSASYGDS |
| | | | | | LTDADRDGTSLTRQPRADSTSV |
| | | | | | GLNFSLPLYSGGATVAATDQAR |
| | | | | | AFYVAASQDYEANYRAITRTVI |
| | | | | | TSYNQVVSDIATYKALEQAVVS |
| | | | | | AQSALKATEAGFEVGTRTIVDV |
| | | | | | LVSTQNLYNAKRNLADVRYRY |
| | | | | | VLSTLRLKQAAGTLTSKDLVAI |
| | | | | | NQGLKAV |
| 1 | WP_125558125.1 | MULTISPECIES: | Pseudoalteromonas | 431 | MKKSILSLFIGVACALSSTASQA |
| | | outer | | | EDLLQVYEIATANDPTVLKAKA |
| | | membrane | | | QADAQKYAQDSALGDLLPSLG |
| | | channel | | | FSMSYTDNDGESTLPGQNDASG |
| | | protein TolC | | | YTVVDTWSDSITRSITLNQTIFS |
| | | | | | MATWQNLTIAEKQAMQALTSY |
| | | | | | EQQQQDLIVRVAQGYFDVLSAL |
| | | | | | DNLEFVQAEKRAIERQLEQTKQ |
| | | | | | RYEVGLTAITDVHEAQAQFDQS |
| | | | | | VANEIVAQNTVETARETLRTIT |
| | | | | | GKYHAKLDKLNTDSFSTVKPN |
| | | | | | RQSNDFITLAKEQNLSLQVAKS |
| | | | | | ALDIAQDQIDLAQAGHYPTLSL |
| | | | | | QASYSDSLGNTEGAELSPRTDG |
| | | | | | TSIGLSFNVPIYSGGKTVAATDQ |
| | | | | | ARALYVAASEDLEASMRNVTR |
| | | | | | SVITSYNQVMSDVATYRALEQA |
| | | | | | VVSAESALQATEAGFEVGTRTI |
| | | | | | VDVLVSTRNLYSAKRNLANLR |
| | | | | | YQYVLSTLKLKQAAGTLNRSD |
| | | | | | LEAINKGLVAG |
| 1 | WP_007376512.1 | outer | Pseudoalteromonas | 432 | MKKNILAALISLSCALGSTAAN |
| | | membrane | sp. Bsw20308 | | AEDLLNVFEIATANDPTVLKAK |
| | | channel | | | AQADAQSYESDRAMSVLLPQIN |
| | | protein TolC | | | LTMGYDKSDATSYQSVGDLAS |
| | | | | | QVQVESDTDQFTRGVSLSQTLF |
| | | | | | DLGAWNSLGIADKQALQASGQ |
| | | | | | YDAAKQNLIVRVAEGYFNVLS |
| | | | | | AVDNLEFIEAEKRAIERQLEQTK |
| | | | | | QRYAVGLTAITDVHEAQAQFD |
| | | | | | NSVAQEIIASNAVETAREQLREI |
| | | | | | TGKYHAKLDFLNTDTFSTTKPA |
| | | | | | KLSSDFIKVAENKNISLQVSKVT |
| | | | | | VDIAKDQIELAKAGHYPKLTLS |
| | | | | | ASYGDSLTDTQRNGVNFDDQP |
| | | | | | RSDSSSVGLNFSVPLYSGGATV |
| | | | | | AATNQARAFYVAASQDYETNY |
| | | | | | RAITRTVITSYNQVVSDIATYKA |
| | | | | | LEQAVVSAQSALKATEAGFEV |
| | | | | | GTRTIVDVLVSTQNLYNAKRNL |
| | | | | | ADVRYRYVLSTLRLKQAAGTL |
| | | | | | TSEDLVAINQGLKAA |
| 1 | WP_002551791.1 | MULTISPECIES: | Pseudomonas | 433 | MLRKLSLVVAVSFASSGLTWA |
| | | TolC | | | ADVPLPTQTGLLNVYQQAVDN |
| | | family outer | | | NADLAASRADYDARKEAVPQA |
| | | membrane | | | RAGLLPNISGSAQSNNTRTSIDQ |
| | | protein | | | PRAVATRSGNVYQATLSQPIFR |
| | | | | | ADRWFQLQAAEASNEQAALEL |
| | | | | | SATEQNLILQAAQSYFSVLRAQ |
| | | | | | DNLASTKAEEAAFKRQLDQAN |
| | | | | | ERFDVGLSDKTDVLQAQASYD |
| | | | | | TSRASRILAKRQVDDAFQALVT |
| | | | | | LTNREYNSIEGIVHTLPVLAPTP |
| | | | | | NDAKAWVDTAAQQNLNLLAS |
| | | | | | NYAVNAAEETLRQRKAGHAPT |
| | | | | | LDAVATYQRGDNDALGFSNPN |
| | | | | | YTGRNYGGDVEQRSIGVQLNIPI |
| | | | | | YSGGLTSSQVREAYARLSQSEQ |
| | | | | | RRESLRRQVVENTRNLHRAVN |
| | | | | | TDVEQVQARKQSIISNQSALEA |
| | | | | | TEIGYQVGTRNIVDVLDAQRQL |
| | | | | | YASVRDYNNTRYDYILDNLRL |
| | | | | | KQAAGTLSPGDLQDLSRYLKA |
| | | | | | DYNPDKDFLPPDLATAAQKNFE |
| | | | | | RPAKR |
| 1 | WP_003095699.1 | MULTISPECIES: | Pseudomonas | 434 | MLRRLSLAAAVAAATGVAWA |
| | | efflux | | | AQPTPLPTKTDLISVYKEAVDN |
| | | transporter | | | NADLAAAQADYLARKEVVPQA |
| | | outer | | | RAGLLPQLGAGARVGDTRIAFD |
| | | membrane | | | ERPATVKRNSQVVQATLSQPLF |
| | | subunit OpmH | | | RADRWFQWQAAKETSDQARLE |
| | | | | | FSATQQDLILRSAETYFTVLRAQ |
| | | | | | DNLATSKAEEAAFKRQLDQAN |
| | | | | | ERFDVGLSDKTDVLEAQASYDT |
| | | | | | ARANRLIAEQRVDDAFQALVTL |
| | | | | | TNRDYSAIEGMRHTLPVVPPAP |
| | | | | | NDAKAWVDTAVQQNLRLLAS |
| | | | | | NYAVNAAEETLRQRKAGHLPT |
| | | | | | LDAVAQYQKGDNDALGFANSA |
| | | | | | ANPLVHYGKYVDERSIGLELNI |
| | | | | | PIYSGGLTSSQVRESYQRLNQSE |
| | | | | | QSREGQRRQVVQDTRNLHRAV |
| | | | | | NTDVEQVQARRQAIISNQSSLE |
| | | | | | ATEIGYQVGTRNIVDVLNAQRQ |
| | | | | | LYAAVRDYNNSRYDYILDTLRL |
| | | | | | KQAAGTLSPADLEALSAYLKQ |
| | | | | | DYDPDKDFLPPDLAKAAAEQL |
| | | | | | QSKPRQQY |
| 1 | WP_003123581.1 | MULTISPECIES: | Pseudomonas | 435 | MLRRLSLAAAVAAATGVAWA |
| | | efflux | | | AQPTPLPTKTDLISVYKEAVDN |
| | | transporter | | | NADLAAAQADYLARKEVVPQA |
| | | outer | | | RAGLLPQLGAGGRIGDSRIALD |
| | | membrane | | | EPAATVKRSSHVVQASLSQPLF |
| | | subunit OpmH | | | RADRWFQWQAAKETSDQAKL |
| | | | | | EFSATQQDLILRSAETYFTVLRA |
| | | | | | QDNLATSKAEEAAFKRQLDQA |
| | | | | | NERFDVGLSDKTDVLEAQASY |
| | | | | | DTARANRLIAEQRVDDAFQAL |
| | | | | | VTLTNRDYSAIEGMRHTLPVVP |
| | | | | | PAPNDAKAWVDTAVQQNLRLL |
| | | | | | ASNYAVNAAEETLRQRKAGHL |
| | | | | | PTLDAVAQYQKGDNDALGFAN |
| | | | | | SAANPLVHYGKDVEERTIGLEL |
| | | | | | NIPIYSGGLTSSQVRESYQRLNQ |
| | | | | | SEQSREGQRRQVVQDTRNLHR |
| | | | | | AVNTDVEQVQARRQAIISNQSS |
| | | | | | LEATEIGYQVGTRNIVDVLNAQ |
| | | | | | RQLYAAVRDYNNSRYDYILDT |
| | | | | | LRLKQAAGTLSPADLEALSAYL |
| | | | | | KQDYDPDKDFLPPDLAKAAAE |
| | | | | | QLQSKPRQQY |
| 1 | WP_003366255.1 | MULTISPECIES: | Pseudomonas | 436 | MLRKLSLVVAVSFASSGLTWA |
| | | TolC | | | AELPMPTQTGLLSVYQQAVDN |
| | | family outer | | | NADLAASRADYDARKEAVPQA |
| | | membrane | | | RAGLLPNISGSAQNSSTRTSIDQ |
| | | protein | | | PRAVATRSGNLYQATLSQPIFR |
| | | | | | ADRWFQLQAAEASNEQAALEL |
| | | | | | SATEQNLILQSAQSYFSVLRAQ |
| | | | | | DNLASTKAEEAAFKRQLDQAN |
| | | | | | ERFDVGLSDKTDVLQAQASYD |
| | | | | | TSRASRLIAQRQVDDAFQALVT |
| | | | | | LTNREYNSIEGIVHTLPVLAPTP |
| | | | | | NDAKAWVDTAAQQNLNLLAS |
| | | | | | NYAVSAAEETLRQRKAGHAPT |
| | | | | | LDAVATYQRGDNDALGFSNPN |
| | | | | | YTGRNYGGDVEQRSIGVQLNIPI |
| | | | | | YSGGLTSSQVREAYARLSQSEQ |
| | | | | | RRESLRRQVVENTRNLHRAVN |
| | | | | | TDVEQVQARKQSIISNQSALEA |
| | | | | | TEIGYQVGTRNIVDVLDAQRQL |
| | | | | | YASVRDYNNTRYDYILDNLRL |
| | | | | | KQAAGTLNPGDLQDLSRYLKA |
| | | | | | DYNPDKDFLPPDLATAAQKNFE |
| | | | | | RPAKR |
| 1 | WP_003438959.1 | MULTISPECIES: | Pseudomonas | 437 | MLRKLSLAIAVSWATNAMALD |
| | | TolC | | | TQAPLSSQNGLVSVYHEAVDN |
| | | family outer | | | NADLAAARADYDARKEVVPQA |
| | | membrane | | | RAGLLPNLSGGAESSNVRTSID |
| | | protein | | | QPSLTTTRSAVVYQATLAQPIFR |
| | | | | | ADRWFQLKAAESVNEQAALQL |
| | | | | | SATEQNLILQTAESYFSVLRAQ |
| | | | | | DTLASTKAEEAAFKRQLDQSRE |
| | | | | | RFDVGLSDKTDVLQSQASYDT |
| | | | | | ARANRLLAQRQVADAFEALITL |
| | | | | | TNRQYNSIEGMRHTLPILPPTPN |
| | | | | | DAKAWVDTAAQQNLNLLASNF |
| | | | | | AVAAAEETLKQRKAGHAPTLD |
| | | | | | AVAQYKTGDNDGFGLSNPNAL |
| | | | | | QQRYSGDVSQRSIGLQLNIPIYS |
| | | | | | GGLTSSQVRESYSRLNQTEQQR |
| | | | | | ESMRRQVVENTRNLHRAVNTD |
| | | | | | VEQVQARKQSIISNQSALEATEI |
| | | | | | GYQVGTRNIVDVLDAQRQLYT |
| | | | | | SVRDYNNSRYDYILDNLRLKQA |
| | | | | | AGTLNPGDLEALSRYLKPDYNP |
| | | | | | DKDFLPPDLAQAAEANMRSPG |
| | | | | | N |
| 1 | WP_009877184.1 | MULTISPECIES: | Pseudomonas | 438 | MLRRLSLAAAVAAATGVAWA |
| | | efflux | | | AQPTPLPTKTDLISVYKEAVDN |
| | | transporter | | | NADLAAAQADYLARKEVVPQA |
| | | outer | | | RAGLLPQLGAGARVGDTRIAFD |
| | | membrane | | | ERPATVKRNSQVVQATLSQPLF |
| | | subunit OpmH | | | RADRWFQWQAAKETSDQARLE |
| | | | | | FSATQQDLILRSAETYFTVLRAQ |
| | | | | | DNLATSKAEEAAFKRQLDQAN |
| | | | | | ERFDVGLSDKTDVLEAQASYDT |
| | | | | | ARANRLIAEQRVDDAFQALVTL |
| | | | | | TNRDYSAIEGMRHTLPVVPPAP |
| | | | | | NDAKAWVDTAVQQNLRLLAS |
| | | | | | NYAVNAAEETLRQRKAGHLPT |
| | | | | | LDAVAQYQKGDNDALGFANSA |
| | | | | | ANPLVHYGKYVDERSIGLELNI |
| | | | | | PIYSGGLTSSQVRESYQRLNQSE |
| | | | | | QSREGQRRQVVQDTRNLHRAV |
| | | | | | NTDVEQVQARRQAIISNQSSLE |
| | | | | | ATEIGYQVGTRNIVDVLNAQRQ |
| | | | | | LYAAVRDYNNSRYDYILDTLRL |
| | | | | | KQAAGTLSPADLEALSAYLKQ |
| | | | | | DYDPDKDFLPPDLAKAASEQLQ |
| | | | | | SKPRQQY |
| 1 | WP_010213649.1 | MULTISPECIES: | Pseudomonas | 439 | MLRKLSLALAVSCATNGMVW |
| | | TolC | | | AAEAPLSANTDLVSVYQEAVD |
| | | family outer | | | NNADLAAARAQYGAQKEVVP |
| | | membrane | | | QARAGLLPNLTGGADINNVRTQ |
| | | protein | | | IDTPAATANRDAHSWRATLSQP |
| | | | | | LFRADRWFQLQAAEATNEQAA |
| | | | | | LQLSATEQNLILQSAENYFAVL |
| | | | | | RAQDNLASTKAEENAFKRQLD |
| | | | | | QSNERFDVGLSDKTDVLQSQAS |
| | | | | | YDTARANRILAQRQVEDAFEAL |
| | | | | | ITLTNRQYNSIQGIVHTLPVLPPL |
| | | | | | PNDAKAWVETAGRQNLNLLAS |
| | | | | | NYAVTAAEETLKQRKAGHAPT |
| | | | | | LDAVAQYEKGDNDALGFSNPN |
| | | | | | AFGTPYRGDVEQRTIGLRLSIPI |
| | | | | | YSGGLTSSQVRESYSRLGQTEQ |
| | | | | | QREGLRRQVVENTRNLHRAVN |
| | | | | | TDVEQVQARRQSIISNQSAVEA |
| | | | | | TEIGYQVGTRNIVDVLDSQRQL |
| | | | | | YASVRNYNNSRYDYILDNLRLK |
| | | | | | QAAGTLNPGDLQDLARYLKAD |
| | | | | | YNPDRDFLPPDLAKAAAEQLK |
| | | | | | ARPGY |
| 1 | WP_010955503.1 | MULTISPECIES: | Pseudomonas | 440 | MLRKLSLAIAVSCASNGVVWA |
| | | TolC | | | ADVPSTVKTDLVSVYQEAVDN |
| | | family outer | | | NADLAAARADYGARREVVPQA |
| | | membrane | | | RAGLLPNLSAGAEMMNTRTKL |
| | | protein | | | DEPSITSNRSGNSWSATLAQPIF |
| | | | | | RADRWFQLQAAEAVNEQAALE |
| | | | | | LSATEQNLILQTAQNYFAVLRA |
| | | | | | QDNLAATKAEEAAFKRQLDQS |
| | | | | | NERFDVGLSDKTDVLQSQASY |
| | | | | | DTARANRIIAERQVQDAFEALV |
| | | | | | TLTNREYTSIQGVVHSLPVQVP |
| | | | | | TPNDAKAWVETAGRQNLNLLA |
| | | | | | TNYAVSAAEETLRQRKAGHAP |
| | | | | | TLDAVAKYQKGDNDSLGFTNP |
| | | | | | SQLGVRYSGDVEQTSVGLQLNI |
| | | | | | PIYSGGLTSSQVREAYQRLSQSE |
| | | | | | QQRESLRRQVVENTRNLHRAV |
| | | | | | NTDVEQVQARKQSIISNQSALE |
| | | | | | ATEIGYQVGTRNIVDVLDAQRQ |
| | | | | | LYTSVRDYNNSRYDYILDNLSL |
| | | | | | KQAAGTLSPQDLQDLKRYLKP |
| | | | | | DYNPDKDFLPPDLAAAAAKNF |
| | | | | | ERRP |
| 1 | WP_011058910.1 | MULTISPECIES: | Pseudomonas | 441 | MLRKLSLAVAVSCASNGMAW |
| | | TolC | | | AAEAPLTTKTDLVSVYQEAVD |
| | | family outer | | | NNADLAAARAQYGAQKEVVP |
| | | membrane | | | QARAGLLPNLSAGADMNNTRT |
| | | protein | | | KFDEPSMTSTRSGNVYQATLAQ |
| | | | | | PLFRADRWFQLQAAKDINEQAS |
| | | | | | LQLSATEQNLILQSAENYFAVL |
| | | | | | RAQDNLASTKAEEAAFKRQLD |
| | | | | | QSNERFDVGLSDKTDVLQSQAS |
| | | | | | YDTARANRILAQRQVDDAFEA |
| | | | | | LITLTNREYNSIQGIVHTLPVLA |
| | | | | | PTPNDAKAWVDTAAKQNLNLL |
| | | | | | ASNYAVSAAEETLKQRKAGHA |
| | | | | | PTLDAVAQYKKGDNDALGFSN |
| | | | | | PNPLTRYGSDVEQRSIGLQLNIPI |
| | | | | | YSGGLTSSQVRESYSRLSQTEQ |
| | | | | | QRESLRRQVVENTRNLHRAVN |
| | | | | | TDVEQVQARRQSIISNQSAVEA |
| | | | | | TEIGYQVGTRNIVDVLDAQRQL |
| | | | | | YTSVRNYNNSRYDYILDNLRLK |
| | | | | | QAAGTLNPGDLQDLSRYLKAD |
| | | | | | YNPDRDFLPPDLAKAAQEQLRA |
| | | | | | RP |
| 1 | WP_011061272.1 | MULTISPECIES: | Pseudomonas | 442 | MSKPMNSRQRALAGWLLVVPL |
| | | TolC | | | LVAPLAQAAPLHRQDLPQDSA |
| | | family outer | | | QHPPAYVPQQRPQALPSDLWR |
| | | membrane | | | VYLDARQNNSELAAAQADQAA |
| | | protein | | | RAEAVPQARAGLLPTLSASADL |
| | | | | | NATSTSLQQPRQDTRRSGTSYQ |
| | | | | | AVLNQPIFRADRWFTLKAAQAE |
| | | | | | DQQAQLELAAAEQKLMFDSAQ |
| | | | | | AYFGLLKAQDALAAAKAEEAA |
| | | | | | LKRQQQLAERGLQLGLSDRTD |
| | | | | | VLQAQAGHDTARANRIVAQKQ |
| | | | | | TDDAFEALDTLTHQQYQAVQG |
| | | | | | LRHDMPVLLPEPNDARRWVDT |
| | | | | | AVRQNLTLQASQFALEATQQTL |
| | | | | | SARKAGHAPTLDAVLRYQTGD |
| | | | | | NDALGYGNSDIRGNGYGGNVE |
| | | | | | QRTVGLQLNIPLFSGGQTRSQV |
| | | | | | REAHQRMNQREYLNDGLRRQV |
| | | | | | VEQTRNLHRGLNSGVDQVQAR |
| | | | | | RQSIISNQGAVLASQMGFQVGT |
| | | | | | RNIVDVLEAQRQLYNAVRQYN |
| | | | | | NSRYDYILDTLRLKQAVGTLSP |
| | | | | | QDLKALCDYLKADYDPDRDFL |
| | | | | | PPEFPRRLAKR |
| 1 | WP_011167720.1 | MULTISPECIES: | Pseudomonas | 443 | MNPFSPLVLVILTCACACGETV |
| | | TolC | | | FAREQAKQHADLLSVYQAASL |
| | | family outer | | | NDAQLSAARHAYNAQREAVPQ |
| | | membrane | | | ARAGLLPNLTAGATSEVTRLER |
| | | protein | | | DEPSLSRERSGTTFRANLSQPLF |
| | | | | | RIDRWFQLKAAQSSVDQAELEL |
| | | | | | SAKEQNLLLTAAQAYFETLRQL |
| | | | | | DAFAAAQAEEAALLRQRDQAQ |
| | | | | | GRLDDGASSITDVYDAQAAYD |
| | | | | | NARANRQLVQRKVDDAYEALE |
| | | | | | RLTKHSYTSIAGMRHQMPIEAPI |
| | | | | | PNDARAWVDIAVRQNLELQAS |
| | | | | | ERAVSAAEQTLSQRKAGHAPTI |
| | | | | | DAVASYRKGDNDSFGYSTPTDF |
| | | | | | GANGYRDNVAQSSIGIELNIPLY |
| | | | | | SGGMTSSQIKESTERLYQSQDE |
| | | | | | QEDRRREVVLNTRNAFRGINAG |
| | | | | | IEQIAARRQSIVSGQKSVEANQV |
| | | | | | GVDVGSRNIADVLNAQRQLYA |
| | | | | | AVRDYNDARYDYILDNLKLKQ |
| | | | | | AAGTLSPDDLRALAAYLKQDY |
| | | | | | DPARDFLPPGV |
| 1 | WP_011332156.1 | MULTISPECIES: | Pseudomonas | 444 | MLRKLSLAVAVSCASTTTVWA |
| | | TolC | | | AEAPLSTKTDLVSVYQEAVDN |
| | | family outer | | | NADLAAARAQYGAQKEVVPQ |
| | | membrane | | | ARAGLLPNLSGGAEVANVRTSI |
| | | protein | | | DQPSAIANRSAHSYQATLAQPL |
| | | | | | FRADRWFQYQAAKDVNEQAA |
| | | | | | LQLSATEQNLILQTAESYFNVLR |
| | | | | | SQDNLASTKAEEAAFKRQLDQS |
| | | | | | NERFDVGLSDKTDVLQSQASY |
| | | | | | DTARANRIVAQRQVDDAFEALI |
| | | | | | TLTNRQYNSIQGIVHTLPILPPAP |
| | | | | | NDAKAWVDTAAKQNLNLLAS |
| | | | | | NYAVSSAEQTLKQRKAGHLPTL |
| | | | | | DAVAKYEKGDNDALGFSNPNA |
| | | | | | FGTPYSGNVEQSTVGLQLNIPIY |
| | | | | | SGGLTSSQVRESYSRLDQTEQQ |
| | | | | | REGLRRQVVENTRNLHRAVNT |
| | | | | | DVEQVQARRQSIISNQSAVEAT |
| | | | | | EIGYQVGTRNIVDVLDAQRQLY |
| | | | | | TSVRNYNNTRYDYILDNLRLKQ |
| | | | | | AAGTLNPGDLQDLTRYLKADY |
| | | | | | NPDKDFLPPDLAKAAEAQLKA |
| | | | | | RP |
| 1 | WP_012053869.1 | MULTISPECIES: | Pseudomonas | 445 | MLRKLSLAIAVSCASNGVVWA |
| | | TolC | | | ADVPSTVKTDLVSVYQEAVDN |
| | | family outer | | | NADLAAARADYGARREVVPQA |
| | | membrane | | | RAGLLPNLSAGAEMMNTRTKL |
| | | protein | | | DEPSMTSNRSGNAWSATLAQPI |
| | | | | | FRADRWFQLQAAEAVNEQAAL |
| | | | | | ELSATEQNLILQTAQNYFAVLR |
| | | | | | AQDNLAATKAEEAAFKRQLDQ |
| | | | | | SNERFDVGLSDKTDVLQSQASY |
| | | | | | DTARANRIIAERQVQDAFEALV |
| | | | | | TLTNREYTAIQGVVHSLPVQVP |
| | | | | | TPNDAKAWVETAGRQNLNLLA |
| | | | | | TNYAVSAAEETLRQRKAGHAP |
| | | | | | TLDAVAKYQKGDNDSLGFTNP |
| | | | | | SQLGVRYSGDVEQTSVGLQLNI |
| | | | | | PIYSGGLTSSQVREAYQRLSQSE |
| | | | | | QQRESLRRQVVENTRNLHRAV |
| | | | | | NTDVEQVQARKQSIISNQSALE |
| | | | | | ATEIGYQVGTRNIVDVLDAQRQ |
| | | | | | LYTSVRDYNNSRYDYILDNLSL |
| | | | | | KQAAGTLSPQDLQDLKRYLKP |
| | | | | | DYNPDKDFLPPDLAAAAAKNF |
| | | | | | ERRP |
| 1 | WP_014716646.1 | MULTISPECIES: | Pseudomonas | 446 | MLRKLSLAVAVSCATNGLVWA |
| | | TolC | | | AEAPLSANTDLVSVYQEAAHN |
| | | family outer | | | NADLAAARAQYGAQKEVVPQ |
| | | membrane | | | ARAGLLPNLSAGADSNNVRTQI |
| | | protein | | | DQPAATANRDAHSWRATLSQP |
| | | | | | LFRADRWFQLQAAQAVNEQAS |
| | | | | | LQLSASEQNLILQSAESYFAVLR |
| | | | | | AQDNLASTKAEENAFKRQLDQ |
| | | | | | SNERFDVGLSDKTDVLQSQASY |
| | | | | | DTARANRILAQRQVDDAFEALI |
| | | | | | TLTNRQYNSIQGIVHTLPVLPPA |
| | | | | | PNDAKAWVETAGRQNLNLLAS |
| | | | | | NYAVTAAEETLRQRKAGHLPT |
| | | | | | LDAVAQYEKGDNDALGFSNPN |
| | | | | | QLPIPYGGDVSQRTVGLRLNIPI |
| | | | | | YSGGLTSSQVRESYSRLDQTEQ |
| | | | | | QREGLRRQVVENTRNLHRAVN |
| | | | | | TDVEQVQARRQSIISNQSAVEA |
| | | | | | TEIGYQVGTRNIVDVLDSQRQL |
| | | | | | YASVRNYNNSRYDYILDNLRLK |
| | | | | | QAAGTLNPGDLQDLARYLKAD |
| | | | | | YNPDKDFLPPDLAKAAAEQLK |
| | | | | | ARPGY |
| 1 | WP_016964919.1 | MULTISPECIES: | Pseudomonas | 447 | MLRKLSLAVAVSCASNGMAW |
| | | TolC | | | AAEAPLTTRTDLVSVYQEALDN |
| | | family outer | | | NADLAAARAQYGAQKEVVPQ |
| | | membrane | | | ARAGLLPNLSAGADINNTRTKF |
| | | protein | | | DEPSMASTRSGNVYQATLAQPL |
| | | | | | FRADRWFQLQAAKDINEQASL |
| | | | | | QLSATEQNLILQSAENYFAVLR |
| | | | | | AQDNLASTKAEEAAFKRQLDQ |
| | | | | | SNERFDVGLSDKTDVLQAQAS |
| | | | | | YDTARANRILAQRQVDDAFEA |
| | | | | | LITLTNREYNSIQGIVHTLPVLA |
| | | | | | PTPNDAKAWVDTAARQNLNLL |
| | | | | | ASNYAVSAAEETLKQRKAGHA |
| | | | | | PTLDAVAQYKKGDNDALGFSN |
| | | | | | PNPLTRYGSDVEQRSIGLQLNIPI |
| | | | | | YSGGLTSSQVRESYSRLSQTEQ |
| | | | | | QRESLRRQVVENTRNLHRAVN |
| | | | | | TDVEQVQARRQSIISNQSAVEA |
| | | | | | TEIGYQVGTRNIVDVLDAQRQL |
| | | | | | YTSVRNYNNSRYDYILDNLRLK |
| | | | | | QAAGTLNPGDLQDLSRYLKAD |
| | | | | | YNPDRDFLPPDLAKAAQEQLK |
| | | | | | ARP |
| 1 | WP_017002004.1 | MULTISPECIES: | Pseudomonas | 448 | MLRRLSLAAAVAAATGVAWA |
| | | efflux | | | AQPTPLPTKTDLISVYKEAVDN |
| | | transporter | | | NADLAAAQADYLARKEVVPQA |
| | | outer | | | RAGLLPQLGAGGRIGDSRIALD |
| | | membrane | | | EPAATVKRSSHVVQASLSQPLF |
| | | subunit OpmH | | | RADRWFQWQAAKETSDQAKL |
| | | | | | EFSATQQDLILRSAETYFTVLRA |
| | | | | | QDNLATSKAEEAAFKRQLDQA |
| | | | | | NERFDVGLSDKTDVLEAQASY |
| | | | | | DTARANRLIAEQRVDDAFQAL |
| | | | | | VTLTNRDYSAIEGMRHTLPVVP |
| | | | | | PAPNDAKAWVDTAVQQNLRLL |
| | | | | | ASNYAVNAAEETLRQRKAGHL |
| | | | | | PTLDAVAQYQKGDNDALGFAN |
| | | | | | SAANPLVHYGKDVEERTIGLEL |
| | | | | | NIPIYSGGLTSSQVRESYQRLNQ |
| | | | | | SEQSREGQRRQVVQDTRNLHR |
| | | | | | AVNTDVEQVQARRQAIISNQSS |
| | | | | | LEATEIGYQVGTRNIVDVLNAQ |
| | | | | | RQLYAAVRDYNNSRYDYILDT |
| | | | | | LRLKQAAGTLSPADLEALSAYL |
| | | | | | KQDYDPDKDFLPPDLAKAASE |
| | | | | | QLQSKPRQQY |
| 1 | WP_017363308.1 | MULTISPECIES: | Pseudomonas | 449 | MLRRLSLAIAVAAASVGLVQA |
| | | TolC | | | AEAPLSSKTDLVTVYQEAAKN |
| | | family outer | | | NADIAAARADYQARREVVPQA |
| | | membrane | | | RAGLLPNLSAGANYGDTRTEID |
| | | protein | | | SPSATVSRSGLVYQANLSQPLF |
| | | | | | RADRWFQLQAAEATSEQAALE |
| | | | | | LSATEQNLILQSAETYFAVLRA |
| | | | | | QDNLASTRAEEAAFKRQLDQA |
| | | | | | NERFDVGLSDKTDVLEAQAGF |
| | | | | | DTARANRLLAERAVDDAFEAL |
| | | | | | VTLTNRDYVAVEGIVHSLPVLA |
| | | | | | PTPNDAKSWVDTAAAQNLNLQ |
| | | | | | ASLYAVNAAEENLRQRKAGHA |
| | | | | | PTLDAVASYQKGDNDSLGFTNS |
| | | | | | GSPLSPPYSGDVSQRTIGLQLNI |
| | | | | | PLYSGGLTSSQVREAYQRLGQT |
| | | | | | EQLRESLRRQVVQNTRNLFRAV |
| | | | | | NTDVETVQARRQSIISNQSALEA |
| | | | | | TEIGYQVGTRNIVDVLDAQRQL |
| | | | | | YSAVRNYNDARYDYILNNLRL |
| | | | | | KQAAGTLSPADLEALGSFLKPD |
| | | | | | YNPDKDFLPPDLASAAEERLQN |
| | | | | | NQDF |
| 1 | WP_021263544.1 | MULTISPECIES: | Pseudomonas | 450 | MLRRLSLAAAVAAATGVAWA |
| | | efflux | | | AQPTPLPTKTDLISVYKEAVDN |
| | | transporter | | | NADLAAAQADYLARKEVVPQA |
| | | outer | | | RAGLLPQLGAGARVGDTRIAFD |
| | | membrane | | | ERPATVKRNSQVVQATLSQPLF |
| | | subunit OpmH | | | RADRWFQWQAAKETSDQARLE |
| | | | | | FSATQQDLILRSAETYFTVLRAQ |
| | | | | | DNLATSKAEEAAFKRQLDQAN |
| | | | | | ERFDVGLSDKTDVLEAQASYDT |
| | | | | | ARANRLIAEQRVDDAFQALVTL |
| | | | | | TNRDYSAIEGMRHTLPVVPPAP |
| | | | | | NDAKAWVDTAVQQNLRLLAS |
| | | | | | NYAVNAAEETLRQRKAGHLPT |
| | | | | | LDAVAQYQKGDNDALGFANSA |
| | | | | | ANPLVHYGKYVDERSIGLELNI |
| | | | | | PIYSGGLTSSQVRESYQRLNQSE |
| | | | | | QSREGQRRQVVQDTRNLHRAV |
| | | | | | NTDVEQVQARRQAIISNQSSLE |
| | | | | | ATEIGYQVGTRNIVDVLNAQRQ |
| | | | | | LYAAVRDYNNSRYDYILDTLRL |
| | | | | | KQAAGTLSPADLEALSAYLKQ |
| | | | | | DYDPDKDFLPPDLAKAAAEQL |
| | | | | | QSRPRQQY |
| 1 | WP_023632667.1 | MULTISPECIES: | Pseudomonas | 451 | MLRKLSLAIAVSCASNGVAWA |
| | | TolC | | | VDTPTTVKTDLVSVYQEAVDN |
| | | family outer | | | NADLAAARADYGARREVVPQA |
| | | membrane | | | RAGLLPNLSAGAEMMNTRTKL |
| | | protein | | | DEPSITSNRSGNAWSATLAQPIF |
| | | | | | RADRWFQLQAAEAVNEQAALE |
| | | | | | LSATEQNLILQSAENYFAVLRA |
| | | | | | QDNLASTKAEEAAFKRQLDQS |
| | | | | | NERFDVGLSDKTDVLQSQASY |
| | | | | | DTARANRIIAERQVQDAFEALIT |
| | | | | | LTNRQYSAIQGVVHTLPVQVPT |
| | | | | | PNDAKAWVETAGRQNLNLQAT |
| | | | | | NHAVAAAEETVRQRKAGHAPT |
| | | | | | LDAVAKYQKGDNDNLGFTNPS |
| | | | | | QNGVRYGGDVEQTSVGLQLNIP |
| | | | | | IYSGGLTSSQVREAYARLTQSE |
| | | | | | QQRESLRRQVVENTRNLHRAV |
| | | | | | NTDVEQVQARKQSIISNQSALE |
| | | | | | ATEIGYQVGTRNIVDVLDAQRQ |
| | | | | | LYTSVRDYNNSRYDYILDNLRL |
| | | | | | KQAAGTLSPQDLQDLGRYLKA |
| | | | | | DYNPDKDFLPPDLAAAAAKNF |
| | | | | | ERRP |
| 1 | WP_028688288.1 | MULTISPECIES: | Pseudomonas | 452 | MLRKLSLAIAVSCASHGVAWA |
| | | TolC | | | VDTPTTVKTDLVSVYQEAVDN |
| | | family outer | | | NADLAAARADYGARREVVPQA |
| | | membrane | | | RAGLLPNLSAGAEMMNTRTKL |
| | | protein | | | DEPSVTSNRSGNAWSATLAQPI |
| | | | | | FRADRWFQLQAAESVNEQAAL |
| | | | | | ELSATEQNLILQSAENYFAVLR |
| | | | | | AQDNLASTKAEEAAFKRQLDQ |
| | | | | | SNERFDVGLSDKTDVLQSQASY |
| | | | | | DTARANRIIAERQVQDAFEALIT |
| | | | | | LTNRQYTAIQGVVHTLPVQVPT |
| | | | | | PNDAKAWVETAGRQNLNLLAT |
| | | | | | NHAVAAAEETVRQRKAGHAPT |
| | | | | | LDAVAKYQKGDNDNLGFTNPS |
| | | | | | QNGVRYGGDVEQTSVGLQLNIP |
| | | | | | IYSGGLTSSQVREAYARLTQSE |
| | | | | | QQRESLRRQVVENTRNLHRAV |
| | | | | | NTDVEQVQARKQSIISNQSALE |
| | | | | | ATEIGYQVGTRNIVDVLDAQRQ |
| | | | | | LYTSVRDYNNSRYDYILDNLRL |
| | | | | | KQAAGTLSPQDLQDLGRYLKA |
| | | | | | DYNPDKDFLPPDLAAAAAKNF |
| | | | | | ERRP |
| 1 | WP_041924407.1 | MULTISPECIES: | Pseudomonas | 453 | MLRKLSLVVAVSFASSGLTWA |
| | | TolC | | | ADMPLPTQTGLLNVYQQAVDN |
| | | family outer | | | NADLAASRADYDARKEAVPQA |
| | | membrane | | | RAGLLPNISGSAQSNNTRTSIDQ |
| | | protein | | | PRTVATRSGNVYQATLSQPIFR |
| | | | | | ADRWFQLQAAEASNEQAALEL |
| | | | | | SATEQNLILQAAQSYFSVLRAQ |
| | | | | | DNLASTKAEEAAFKRQLDQAN |
| | | | | | ERFDVGLSDKTDVLQAQASYD |
| | | | | | TSRASRILAKRQVDDAFQALVT |
| | | | | | LTNREYNSIEGIVHTLPVLAPTP |
| | | | | | NDAKAWVDTAAQQNLNLLAS |
| | | | | | NYAVSAAEETLRQRKAGHAPT |
| | | | | | LDAVATYQRGDNDALGFSNPN |
| | | | | | YTGRNYGGDVEQRSIGVQLNIPI |
| | | | | | YSGGLTSSQVREAYARLSQSEQ |
| | | | | | RRESLRRQVVENTRNLHRAVN |
| | | | | | TDVEQVQARKQSIISNQSALEA |
| | | | | | TEIGYQVGTRNIVDVLDAQRQL |
| | | | | | YASVRDYNNTRYDYILDNLRL |
| | | | | | KQAAGTLNPGDLQDLSRYLKA |
| | | | | | DYNPDKDFLPPDLATAAQKNFE |
| | | | | | RPAKR |
| 1 | WP_045633175.1 | MULTISPECIES: | Pseudomonas | 454 | MLRRLPLALAVATLTAGGAWA |
| | | TolC | | | QETVPLASKTDLVSVYQQAVN |
| | | family outer | | | NNADLAAARAQFRARQEVVPQ |
| | | membrane | | | ARAGLLPNLSAGAELSDTETDV |
| | | protein | | | DTSMGSTSLSRSGTAYQATLSQ |
| | | | | | PIFRADRWFQLKAAEAVSEQAA |
| | | | | | IEYSIAEQDLILQSAQAYFAVLR |
| | | | | | AQDNLAAVKAEEAAFKRQLDQ |
| | | | | | ANERFEVGLSDKTDVLEAQAGF |
| | | | | | DTARANRSIAQRQVEDAFQALF |
| | | | | | TLTNREYVALEGIRHTLPVLAP |
| | | | | | VPNEAKAWVDTATRQNLNLLA |
| | | | | | VNYAVTAAEETLRQRRSGHAP |
| | | | | | TLDAVASYRKGDNDALGFSNT |
| | | | | | QSIPGFDMPRYTGDVEQRSIGL |
| | | | | | QLNIPLYSGGLTTSQSREAYYQ |
| | | | | | LSQSEQQRESLRRQVVEATRNL |
| | | | | | HRAVNTDIEQVQARRQSIISNQS |
| | | | | | ALEATEIGYQVGTRNIVDVLDT |
| | | | | | QRRLYAAVRDYNNARYDYILD |
| | | | | | NLRLKQAAGTLNPDDLQQLAA |
| | | | | | YLKPDYNPDTDFLPPDLPQTIGQ |
| | | | | | PVRVDY |
| 1 | WP_046070623.1 | MULTISPECIES: | Pseudomonas | 455 | MLRKLSLAVAVSCATNGMVW |
| | | TolC | | | AAEAPLSTNTDLVSVYQEAAH |
| | | family outer | | | NNADLAAARAQYGAQKEVVP |
| | | membrane | | | QARAGLLPNLSAGADSNNVRT |
| | | protein | | | QIDQPAATANRDAHSWRATLS |
| | | | | | QPLFRADRWFQLQAAEAVNEQ |
| | | | | | ASLQLSATEQNLILQSAESYFAV |
| | | | | | LRAQDNLASTKAEENAFKRQL |
| | | | | | DQSNERFDVGLSDKTDVLQSQ |
| | | | | | ASYDTARANRIVAQRQVDDAF |
| | | | | | QALITLTNRQYNSIQGIVHTLPV |
| | | | | | LPPAPNDAKAWVETAGRQNLN |
| | | | | | LLASNYAVTAAEETLRQRKAG |
| | | | | | HLPTLDAVAQYEKGDNDALGF |
| | | | | | SNPNQLPIPYGGDVSQRTIGLRL |
| | | | | | NIPIYSGGLTSSQVRESYSRLDQ |
| | | | | | TEQQREGLRRQVVENTRNLHR |
| | | | | | AVNTDVEQVQARRQSIISNQSA |
| | | | | | VEATEIGYQVGTRNIVDVLDAQ |
| | | | | | RQLYSSVRNYNNSRYDYILDNL |
| | | | | | RLKQAAGTLNPGDLQDLARYL |
| | | | | | KADYNPDKDFLPPDLAKAAAE |
| | | | | | QLKARPGY |
| 1 | WP_047273094.1 | MULTISPECIES: | Pseudomonas | 456 | MLRKLSLAIAVSCATNVMAME |
| | | TolC | | | TQAPLASQTGLVSVYHEAVDN |
| | | family outer | | | NADLAAARADYAARKEVVPQA |
| | | membrane | | | RAGLLPNLSGGAESSSVRTSIDQ |
| | | protein | | | PSLTTTRSAVVYQATLAQPIFRI |
| | | | | | DRWFQLKAAESVNEQAALQLS |
| | | | | | ATEQNLILQTAESYFSVLRAQD |
| | | | | | TLAATKAEEAAFKRQLDQSRER |
| | | | | | FDVGLSDKTDVLQSQASYDTSR |
| | | | | | ANRIVAQRQVDDAFEALITLTN |
| | | | | | RQYNSIEGMRHTLPILPPSPNNA |
| | | | | | KIWVETAAQQNLNLLASNFAV |
| | | | | | DAAQETLKQRKAGHAPTLDAV |
| | | | | | AQYKTGDNDGFGLSNPNALQQ |
| | | | | | RYSGDVSQRSIGIQLNIPIYSGGL |
| | | | | | TSSQVRESYSRLDQTEQQREGL |
| | | | | | RRQVVENTRNLHRAVNTDVEQ |
| | | | | | VQARKQSIISNQSALEATEIGYQ |
| | | | | | VGTRNIVDVLDAQRQLYNSVR |
| | | | | | DYNNTRYDYILDNLRLKQVAG |
| | | | | | TLNPGDLDALSRYLKADYNPD |
| | | | | | KDFLPPDLAQAAEANMRSPGN |
| 1 | WP_054091587.1 | MULTISPECIES: | Pseudomonas | 457 | MLRKLSLVVAVSFASSGLTWA |
| | | TolC | | | AELPMPTQTGLLSVYQQAVDN |
| | | family outer | | | NADLAASRADYDARKEAVPQA |
| | | membrane | | | RAGLLPNISGSAQNSSTRTSIDQ |
| | | protein | | | PRAVASRSGNLYQATLSQPIFR |
| | | | | | ADRWFQLQAAEASNEQAALEL |
| | | | | | SATEQNLILQSAQSYFSVLRAQ |
| | | | | | DNLASTKAEEAAFKRQLDQAN |
| | | | | | ERFDVGLSDKTDVLQAQASYD |
| | | | | | TSRASRLLAQRQVEDAFQALVT |
| | | | | | LTNREYNSIEGIVHTLPVLAPTP |
| | | | | | NDAKAWVDTAAQQNLNLLAS |
| | | | | | NYAVNAAEETLRQRKAGHAPT |
| | | | | | LDAVATYQRGDNDALGFSNPN |
| | | | | | YTGRNYGGDVEQRSIGVQLNIPI |
| | | | | | YSGGLTSSQVREAYARLSQSEQ |
| | | | | | RRESLRRQVVENTRNLHRAVN |
| | | | | | TDVEQVQARKQSIISNQSALEA |
| | | | | | TEIGYQVGTRNIVDVLDAQRQL |
| | | | | | YASVRDYNNTRYDYILDNLRL |
| | | | | | KQAAGTLNPGDLQDLSRYLKA |
| | | | | | DYNPDKDFLPPDLATAAQKNFE |
| | | | | | RPAKR |
| 1 | WP_058543054.1 | MULTISPECIES: | Pseudomonas | 458 | MLRKLSLALAVSCASNGMAWA |
| | | TolC | | | AEAPLTTKTDLVSVYQEAVDN |
| | | family outer | | | NADLAAARAQYGAQKEVVPQ |
| | | membrane | | | ARAGLLPNLSAGADLNNTRTEL |
| | | protein | | | DQPAMTANRSSTVYQATLSQPI |
| | | | | | FRADRWFQLQAAKSVDEQASL |
| | | | | | QLSATEQNMILQSAESYFNVLR |
| | | | | | SQDNLASTKAEEAAFKRQLDQS |
| | | | | | NERFDVGLSDKTDVLQSQASY |
| | | | | | DTARANRILAQRQVDDAFEALI |
| | | | | | TLTNRQYNSLQGIVHTLPILPPA |
| | | | | | PNDAKAWVDTAAKQNLNLLAS |
| | | | | | NYAVTAAEDTLRQRKAGHAPT |
| | | | | | LDAIAQYKKGDNDGLGFTNPSL |
| | | | | | TGQRYGGDAEQRTIGLQLSIPL |
| | | | | | YSGGLTSSQVRESYSQLTQTEQ |
| | | | | | QREALRRQVVENTRNLHRAVN |
| | | | | | TDVEQVQARRQSIISNQSAVEA |
| | | | | | TEIGYQVGTRNIVDVLDAQRQL |
| | | | | | YTSVRNYNNARYDYILDNLRL |
| | | | | | KQAAGTLSPADLQDLSRYLKPD |
| | | | | | YNPDQDFLPPDLAQAAAAQLRS |
| | | | | | RPAQ |
| 1 | WP_058604002.1 | MULTISPECIES: | Pseudomonas | 459 | MLRKLSLALAVSCASQTMAWA |
| | | TolC | | | ADVPATVKTDLVSVYQEAVQN |
| | | family outer | | | NADLAAARADYGARREIVPQA |
| | | membrane | | | RAGLLPNLSAGAEMQSTRTKLD |
| | | protein | | | EPSITSNRSGNAWSATLAQPIFR |
| | | | | | ADRWFQLQAAEAVNEQAALEL |
| | | | | | SATEQNLILQTAQSYFAVLRAQ |
| | | | | | DNLAASKAEEAALKRQLDQSN |
| | | | | | ERFDVGLSDKTDVLQSQASYDT |
| | | | | | ARANRIIADRQVQDAFEALVTL |
| | | | | | TNREYTAIQGVVHSLPVQVPVP |
| | | | | | NDAKAWVETAGRQNLNLLATN |
| | | | | | YAVSAAEETLRQRKAGHAPTV |
| | | | | | DAVAKYQKGDNDNLGFSNPSP |
| | | | | | LGVRYGGDVEQTTVGLQLNIPI |
| | | | | | YSGGLTSSQVREAYQRLSQSEQ |
| | | | | | QRESLRRQVVENTRNLHRAVN |
| | | | | | TDVEQVQARKQSIISNQSALEA |
| | | | | | TEIGYQVGTRNIVDVLDAQRQL |
| | | | | | YTSVRDYNNSRYAYILDNLSLK |
| | | | | | QAAGTLSPQDLQDLKRYLKPD |
| | | | | | YNPDKDFLPPDLAAAAAKNFER |
| | | | | | RP |
| 1 | WP_064615998.1 | MULTISPECIES: | Pseudomonas | 460 | MLRKLSLALAVSCASNGMAWA |
| | | TolC | | | AEAPLSTKTDLVSVYQEAVDN |
| | | family outer | | | NADIAAARAQYGAQKEVVPQA |
| | | membrane | | | RAGLLPNLSAGANMNDTRTAL |
| | | protein | | | DQPSVTSNRSGTVYQATLSQPIF |
| | | | | | RADRWFQFQAAKDVNEQAAL |
| | | | | | QLSATEQNLILQSAESYFNVLRS |
| | | | | | QDNLASTKAEEAAFKRQLDQS |
| | | | | | NERFDVGLSDKTDVLQSQASY |
| | | | | | DTARANRIVAQRQVDDAFEALI |
| | | | | | TLTNRQYNSIQGIVHTLPILPPAP |
| | | | | | NDAKAWVDTAAKQNLNLLAS |
| | | | | | NYAVSAAEETLKQRKAGHAPT |
| | | | | | LDAVAQYRKGDNDALGFTNPS |
| | | | | | ATGVHYGGDVEQRTVGLQLSIP |
| | | | | | IYSGGLTSSQVRESYSRLDQTEQ |
| | | | | | QREALRRQVVENTRNLHRAVN |
| | | | | | TDVEQVQARRQSIISNQSAVEA |
| | | | | | TEIGYQVGTRNIVDVLDAQRQL |
| | | | | | YTSVRNYNNTRYDYILDNLRLK |
| | | | | | QAAGTLNPGDLQDLTRYLKAD |
| | | | | | YNPDKDFLPPDLAKAAAEQLK |
| | | | | | ARP |
| 1 | WP_069898311.1 | MULTISPECIES: | Pseudomonas | 461 | MLRRLTLALAVASASVGSAWA |
| | | TolC | | | EQAHVPLTTKTDLVSVYNEAA |
| | | family outer | | | NNNADLAAARADYQARREVVP |
| | | membrane | | | QARAGLLPNLTGGATVGNTRTS |
| | | protein | | | LDQPSTTLSRSGTSYQANLSQPL |
| | | | | | FRADRWYQLQAAEATSEQAAL |
| | | | | | DLSATEQNLILSSAETYFAVLRA |
| | | | | | QDTLASTKAEEAAFKRQLDQSN |
| | | | | | ERFEVGLSDKTDVLEAQAGYD |
| | | | | | TARANRIIAQRQVDDAFQALMT |
| | | | | | LTNRDYTSIEGIKHTLPILAPTPN |
| | | | | | DAKAWVDTAAAQNLNLQASN |
| | | | | | FAVDAANETLRQRKAGHAPTL |
| | | | | | DAVASYEQGDNDSLGFTNTGIP |
| | | | | | TGAQPYGSDVSQTTLGLQLNIPI |
| | | | | | YSGGLTSSQVREAYQRLDQTEQ |
| | | | | | LRESLRRKIVQDTRNFHRAVNT |
| | | | | | DVETVQARKQSIISNQSALEAT |
| | | | | | QIGYQVGTRNIVDVLDAQRQLY |
| | | | | | SSVRNYNDARYDYILNNLRLKQ |
| | | | | | AAGTLSPGDLEALGAYLKPDY |
| | | | | | NPDKDFLPPDLAKAAEAQMHG |
| | | | | | NPQY |
| 1 | WP_106732497.1 | MULTISPECIES: | Pseudomonas | 462 | MLRRLSLAIAVAAASVGLVQA |
| | | TolC | | | AEAPLSSKTDLVTVYQEAAQN |
| | | family outer | | | NADIAAARADYQARREVVPQA |
| | | membrane | | | RAGLLPNLSAGANYGDTRTEID |
| | | protein | | | SPSATISRSGLVYQANLSQPLFR |
| | | | | | ADRWFQLQAAEATSEQAALEL |
| | | | | | SATEQNLILQSAETYFAVLRAQ |
| | | | | | DNLASTRAEEAAFKRQLDQAN |
| | | | | | ERFDVGLSDKTDVLEAQAGFDT |
| | | | | | ARANRLLAERAVDDAFEALVT |
| | | | | | LTNRDYVAVEGIVHTLPVLAPT |
| | | | | | PNDAKSWVDTAAAQNLNLQAS |
| | | | | | LYAVNAAEENLRQRKAGHAPT |
| | | | | | LDAVASYQQGDNDSLGFTNSG |
| | | | | | AFGAPRYSGDVSQRSIGLQLNIP |
| | | | | | LYSGGLTSSQVREAYQRLGQTE |
| | | | | | QLRESLRRQVVQNTRNLFRAV |
| | | | | | NTDVETVQARRQSIISNQSALEA |
| | | | | | TEIGYQVGTRNIVDVLDAQRQL |
| | | | | | YSAVRNYNDARYDYILNNLRL |
| | | | | | KQAAGTLSPADLEALGSFLKPD |
| | | | | | YNPDKDFLPPDLASAAEERLKN |
| | | | | | NQDF |
| 1 | WP_019726965.1 | efflux | Pseudomonas | 463 | MLRRLSLAAAVAAATGVAWA |
| | | transporter | aeruginosa | | AQPTPLPTKTDLISVYKEAVDN |
| | | outer | | | NADLAAAQADYLARKEVVPQA |
| | | membrane | | | RAGLLPQLGAGGRIGDSRIALD |
| | | subunit OpmH | | | EPAATVKRSSHVVQASLSQPLF |
| | | | | | RADRWFQWQAARETSDQAKLE |
| | | | | | FSATQQDLILRSAETYFTVLRAQ |
| | | | | | DNLATSKAEEAAFKRQLDQAN |
| | | | | | ERFDVGLSDKTDVLEAQASYDT |
| | | | | | ARANRLIAEQRVDDAFQALVTL |
| | | | | | TNRDYSAIEGMRHTLPVVPPAP |
| | | | | | NDAKAWVDTAVQQNLRLLAS |
| | | | | | NYAVNAAEETLRQRKAGHLPT |
| | | | | | LDAVAQYQKGDNDALGFANSA |
| | | | | | ANPLVHYGKDVEERTIGLELNIP |
| | | | | | IYSGGLTSSQVRESYQRLNQSE |
| | | | | | QSREGQRRQVVQDTRNLHRAV |
| | | | | | NTDVEQVQARRQAIISNQSSLE |
| | | | | | ATEIGYQVGTRNIVDVLNAQRQ |
| | | | | | LYAAVRDYNNSRYDYILDTLRL |
| | | | | | KQAAGTLSPADLEALSAYLKQ |
| | | | | | DYDPDKDFLPPDLAKAAAEQL |
| | | | | | QSKPRQQY |
| 1 | WP_023099992.1 | efflux | Pseudomonas | 464 | MLRRLSLAAAVAAATGVAWA |
| | | transporter | aeruginosa | | AQPTPLPTKTDLISVYKEAVDN |
| | | outer | | | NADLAAAQADYLARKEVVPQA |
| | | membrane | | | RAGLLPQLGAGGRIGDSRIALD |
| | | subunit OpmH | | | EPAATVKRSSHVVQASLSQPLF |
| | | | | | RADRWFQWQAAKETSDQAKL |
| | | | | | EFSATQQDLILRSAETYFTVLRA |
| | | | | | QDNLATSKAEEAAFKRQLDQA |
| | | | | | NERFDVGLSDKTDVLEAQASY |
| | | | | | DTARANRLIAEQRVDDAFQAL |
| | | | | | VTLTNRDYSAIEGMRHTLPVVP |
| | | | | | PAPNDAKAWVDTAVQQNLRLL |
| | | | | | ASNYAVNAAEETLRQRKAGHL |
| | | | | | PTLDAVAQYQKGDNDALGFAN |
| | | | | | SAANPLVHYGKDVEERTIGLEL |
| | | | | | NIPIYTGGLTSSQVRESYQRLNQ |
| | | | | | SEQSREGQRRQVVQDTRNLHR |
| | | | | | AVNTDVEQVQARRQAIISNQSS |
| | | | | | LEATEIGYQVGTRNIVDVLNAQ |
| | | | | | RQLYAAVRDYNNSRYDYILDT |
| | | | | | LRLKQAAGTLSPADLEALSAYL |
| | | | | | KQDYDPDKDFLPPDLAKAAAE |
| | | | | | QLQSKPRQQY |
| 1 | WP_033936910.1 | efflux | Pseudomonas | 465 | MLRRLSLAAAVAAATGVAWA |
| | | transporter | aeruginosa | | ALPTPLPTKTDLISVYKEAVDN |
| | | outer | | | NADLAAAQADYLARKEVVPQA |
| | | membrane | | | RAGLLPQLGAGGRIGDSRIALD |
| | | subunit OpmH | | | EPAATVKRSSHVVQASLSQPLF |
| | | | | | RADRWFQWQAAKETSDQAKL |
| | | | | | EFSATQQDLILRSAETYFTVLRA |
| | | | | | QDNLATSKAEEAAFKRQLDQA |
| | | | | | NERFDVGLSDKTDVLEAQASY |
| | | | | | DTARANRLIAEQRVDDAFQAL |
| | | | | | VTLTNRDYSAIEGMRHTLPVVP |
| | | | | | PAPNDAKAWVDTAVQQNLRLL |
| | | | | | ASNYAVNAAEETLRQRKAGHL |
| | | | | | PTLDAVAQYQKGDNDALGFAN |
| | | | | | SAANPLVHYGKDVEERTIGLEL |
| | | | | | NIPIYSGGLTSSQVRESYQRLNQ |
| | | | | | SEQSREGQRRQVVQDTRNLHR |
| | | | | | AVNTDVEQVQARRQAIISNQSS |
| | | | | | LEATEIGYQVGTRNIVDVLNAQ |
| | | | | | RQLYAAVRDYNNSRYDYILDT |
| | | | | | LRLKQAAGTLSPADLEALSAYL |
| | | | | | KQDYDPDKDFLPPDLAKAASE |
| | | | | | QLQSKPRQQY |
| 1 | WP_193034256.1 | TolC family | Pseudomonas | 466 | MSRAQRVLLFACLFPSFVFSAP |
| | | outer | brassicacearum | | TAERAELMAVYRQAVSHNSDL |
| | | membrane | | | AAARADYAAQQELVPQARANL |
| | | protein | | | LPVISTGATFEATRLSRDEPALE |
| | | | | | RGRSGNTIQANLKQPLVNAAF |
| | | | | | WYEFKAAKASTVQAALELSAK |
| | | | | | EQALILQTAQAYFETLRATDEL |
| | | | | | AASEAEETALKQQMDQAQARL |
| | | | | | KGGLSSITDVLDAESAFDNAQA |
| | | | | | NRQLAQRKVEDAFEQLVHLTN |
| | | | | | QQYTSIEGMQHQLPVLAPIPND |
| | | | | | TKSWVEGAVEQNLMLLASNYA |
| | | | | | VEAAEERLRQRQSGHAPTVDA |
| | | | | | VASYRKGDNDSFGYSNPTDFGR |
| | | | | | DGYRGNVAQSSIAVEVTVPLYS |
| | | | | | GGRVSSQAHEAYQRLTQSEELR |
| | | | | | ESQRREVVLDTRNYLRAVNSDI |
| | | | | | QQIKARRQTILSSQKSLKASKV |
| | | | | | GVDIGTRNTVDVLNAQRQLFKS |
| | | | | | VRDYNDARYDYILNNLRLKQA |
| | | | | | AGTLSADDLQQLAEYLKADYQ |
| | | | | | PQRDFLPPELM |
| 1 | WP_005888461.1 | TolC family | Pseudomonas | 467 | MLRKLSLVVAVSFASSGLTWA |
| | | outer | coronafaciens | | ADLPMPTRTGLLNVYRQAVDN |
| | | membrane | | | NADLAASRADYDARKEAVPQA |
| | | protein | | | RAGLLPNISGSVQNTNTRTSIDR |
| | | | | | PSAVATRSGTVYQATLSQPIFRA |
| | | | | | DRWFQLQAAEAVNEQAALELS |
| | | | | | ATEQNLILQSAQSYFSVLRAQD |
| | | | | | NLASTKAEEAAFKRQLDQANE |
| | | | | | RFDVGLSDKTDVLQAQASYDT |
| | | | | | SRASRLLAKRQVDDAFQALVTL |
| | | | | | TNREYNSIEGIVHTLPVLAPTPN |
| | | | | | DAKAWVDTAAQQNLNLLASN |
| | | | | | YAVSAAEETLRQRKAGHAPTL |
| | | | | | DAVATYQRGDNDSLGFNNPNY |
| | | | | | TGQNYGGDVEQRSIGVQLNIPI |
| | | | | | YSGGLTSSQVREAYSRLSQTEQ |
| | | | | | RRESLRRQVVENTRNLHRAVN |
| | | | | | TDVEQVQARKQSIISNQSALEA |
| | | | | | TEIGYQVGTRNIVDVLDAQRQL |
| | | | | | YASVRDYNNTRYDYILDNLRL |
| | | | | | KQAAGTLNPGDLQDLSRYLKP |
| | | | | | DYNPDKDFLPPDLATAAQKNFE |
| | | | | | RPAQR |
| 1 | WP_122357738.1 | TolC family | Pseudomonas | 468 | MLSQGRTVYVDAFKTTLLFLFS |
| | | outer | coronafaciens | | IVALPLLAAPAPADKSAVYGAG |
| | | membrane | | | SQGRDMPRQRADLLGVYEAAS |
| | | protein | | | HNDARLSAARHAYRAEIEAVPQ |
| | | | | | ARAGLLPSLTAGATTEVTGLKR |
| | | | | | DEPALSRERSGTTVRANLSQPLF |
| | | | | | RIDRWFQLKAAQSSVIQAELEL |
| | | | | | AAQEQNLILTAAQTYFEILRQL |
| | | | | | DAFAAAQAEEAALLRQRDQAQ |
| | | | | | GRLEDGASSITDVYDAQAAYD |
| | | | | | NARADRQQFQRKVDDAYEALE |
| | | | | | RLTRRTYSAIAGMGHQLPVEAP |
| | | | | | VPNDARAWVDTAVRQNLELQA |
| | | | | | SEYAVSAATHTLSQRKSGHAPT |
| | | | | | IDAVASYRKGDNDSFGYSNPTD |
| | | | | | FGSNGYRDRVAQSSVGIELNIPL |
| | | | | | YSGGMTNSQIKESTERLYQSQD |
| | | | | | EQEDRRREVVLNTRNAFRGINA |
| | | | | | GIEQIIARRQSIVSGQKSVEANQ |
| | | | | | VGVDLGSRNIADVLNAQRQLY |
| | | | | | AAVRDYNDARYDYILDNLKLK |
| | | | | | QAAGTLSPDDLKALARYLKQD |
| | | | | | YDPARDFLPTGS |
| 1 | WP_036995489.1 | TolC family | Pseudomonas | 469 | MLRKLSLALAVSCATNGMAW |
| | | outer | donghuensis | | AADVPLSAKTDLVSVYQEAVN |
| | | membrane | | | NNADLAAARANYGAQKEVVP |
| | | protein | | | QARAGLLPNLSAGAEMMSNRT |
| | | | | | KIDSPSVTSNRSGNSWQATLAQ |
| | | | | | PIFRADRWFQLQAAEAVNEQA |
| | | | | | ALELSATEQNLILQSAENYFAV |
| | | | | | LRAQDTLASTKAEEAAFKRQLD |
| | | | | | QSNERFDVGLSDKTDVLQSQAS |
| | | | | | YDTARANRIVAQRQVDDAFEA |
| | | | | | LITLTNRDYNSVQGIVHTLPVQ |
| | | | | | VPVPNDAKAWVETAGKQNLNL |
| | | | | | LATNYAVSAAEETLRQRKAGH |
| | | | | | APTVDAVARYQKGDNDSLGFT |
| | | | | | NPNLTGQNYSGDVEQTSIGLQL |
| | | | | | NIPIYSGGLTSSQVREAYQRLSQ |
| | | | | | SEQQRESLRRQVVENTRNLHRA |
| | | | | | VNTDVEQVQARKQSIISNQSAL |
| | | | | | EATEIGYQVGTRNIVDVLDAQR |
| | | | | | QLYTSVRDYNNTRYDYILDNLR |
| | | | | | LKQAAGTLSPQDLTDLSRYLKS |
| | | | | | DYNPDKDFLPPDLAAAAKANF |
| | | | | | EALP |
| 1 | WP_019437565.1 | TolC family | Pseudomonas | 470 | MLRKLSLAIAVSCASNGVVWA |
| | | outer | putida | | ADVPSTVKTDLVSVYQEAVDN |
| | | membrane | | | NADLAAARADYGARREVVPQA |
| | | protein | | | RAGLLPNLSAGAEMMNTRTKL |
| | | | | | DEPSITSNRSGNSWSATLAQPIF |
| | | | | | RADRWFQLQAAEAVNEQAALE |
| | | | | | LSATEQNLILQTAQNYFAVLRA |
| | | | | | QDNLAATKAEEAAFKRQLDQS |
| | | | | | NERFDVGLSDKTDVLQSQASY |
| | | | | | DTARANRIIAERQVQDAFEALV |
| | | | | | TLTNREYTAIQGVVHSLPVQVP |
| | | | | | TPNDAKAWVETAGRQNLNLLA |
| | | | | | TNYAVSAAEETLRQRKAGHAP |
| | | | | | TLDAVAKYQKGDNDSLGFTNP |
| | | | | | SQLGVRYSGDVEQTSVGLQLNI |
| | | | | | PIYSGGLTSSQVREAYQRLSQSE |
| | | | | | QQRESLRRQVVENTRNLHRAV |
| | | | | | NTDVEQVQARKQSIISNQSALE |
| | | | | | ATEIGYQVGTRNIVDVLDAQRQ |
| | | | | | LYTSVRDYNNSRYDYILDNLSL |
| | | | | | KQAAGTLSPQDLQDLKRYLKP |
| | | | | | DYNPDKDFLPPDLAAAAAKNF |
| | | | | | ERRP |
| 1 | WP_063423397.1 | TolC family | Pseudomonas | 471 | MLRKLSLAIAVSCASNGVAWA |
| | | outer | putida | | VDVPSTVKTDLVSVYQEAVDN |
| | | membrane | | | NADLAAARADYGARREVVPQA |
| | | protein | | | RAGLLPNLSADAEMLNTRTKL |
| | | | | | DEPSITSNRSGNSWSATLAQPIF |
| | | | | | RADRWFQLQAAEAVNEQAALE |
| | | | | | LSATEQNLILQTAQNYFAVLRA |
| | | | | | QDNLAATKAEEAAFKRQLDQS |
| | | | | | NERFDVGLSDKTDVLQSQASY |
| | | | | | DTARANRIIAERQVQDAFEALV |
| | | | | | TLTNREYTSIQGVVHTLPVQVP |
| | | | | | TPNDAKAWVETAGRQNLNLLA |
| | | | | | TNYAVSAAEETLRQRKAGHAP |
| | | | | | TLDAVAKYQKGDNDNLGFTNP |
| | | | | | APQQNVRYGGDVEQTSVGLQL |
| | | | | | NIPIYSGGLTSSQVREAYQRLSQ |
| | | | | | SEQQRESLRRQVVENTRNLHRA |
| | | | | | VNTDVEQVQARKQSIISNQSAL |
| | | | | | EATEIGYQVGTRNIVDVLDAQR |
| | | | | | QLYTSVRDYNNSRYDYILDNLS |
| | | | | | LKQAAGTLSPQDLQDLKRYLKP |
| | | | | | DYNPDKDFLPPDLAAAAAKNF |
| | | | | | ERRP |
| 1 | WP_159412348.1 | TolC family | Pseudomonas | 472 | MLRKLSLAIAVSCASNGVVWA |
| | | outer | putida | | ADMPTTVKTDLVSVYQEAVDN |
| | | membrane | | | NADLAAARADYGAQKEVVPQ |
| | | protein | | | ARAGLLPNLSAGAEMQNTRTK |
| | | | | | LDQPSATINRSGNSWSATLSQPI |
| | | | | | FRADRWFQLQAAEAVNEQAAL |
| | | | | | QLSATEQNLILQTAQDYFTVLR |
| | | | | | AQDNLAATKAEEAAFKRQLDQ |
| | | | | | SNERFDVGLSDKTDVLQSQASY |
| | | | | | DTARANRIVAERQVQDAFEAL |
| | | | | | VTLTNREYSSIQGVVHTLPVKV |
| | | | | | PTPNDAKAWVETAGRQNLNLL |
| | | | | | ATNHAVAAAEETLRQRKAGHA |
| | | | | | PTLDAVAQYQKGDNDNLGFSN |
| | | | | | SAALPNVHYSGDVEQTTVGLQ |
| | | | | | LNIPIYSGGLTSSQVREAYQRLS |
| | | | | | QSEQQRESLRRQVVENTRNLHR |
| | | | | | AVNTDVEQVQARKQSIISNQSA |
| | | | | | LEATEIGYQVGTRNIVDVLDAQ |
| | | | | | RQLYTSVRDYNNSRYDYIIDNL |
| | | | | | SLKQAAGTLSPQDLQDLKQYL |
| | | | | | KPDYNPDKDFLPPDLAAAAAK |
| | | | | | NFERRP |
| 1 | WP_062574713.1 | MULTISPECIES: | Pseudomonas | 473 | MLRKTLLPLLFLAPLAQAVAAE |
| | | TolC | putida group | | RATLMSVYAQVVEHNSDMAA |
| | | family outer | | | ARENYLARREAVPLARARLLPQ |
| | | membrane | | | VALDAEAGDVRSDRHRVEQRS |
| | | protein | | | GSLYRLSLDQPLFDLSRWFDFK |
| | | | | | AAQSENEQAELDFSAFQQQLIL |
| | | | | | DTASRYFDTLLAEDNLATAKAE |
| | | | | | LRAFDRQLQQTRLSYDAGLSDQ |
| | | | | | NDMLSAQASFDRASANLIDSQR |
| | | | | | RSEDAYQALMRLTGQAQPALA |
| | | | | | GIRHSLPVQAPVPARAEAWVEQ |
| | | | | | AMAQNLRLRASQAAVSQAGQT |
| | | | | | LRSSKADHLPTFNLSMGYAEGD |
| | | | | | SDLMDSSAHFGQRSGNQRDSS |
| | | | | | VMVQMKLPLFSGGGTSARVRQ |
| | | | | | ATHELARTEYGRTSLEREVLEG |
| | | | | | SRNAFRAVVSDVEQVRARRQSI |
| | | | | | ISSQGSLRATEAGYEVGSRNIVD |
| | | | | | ILDAQRDLYNAVRDYNVSRYA |
| | | | | | YIVDSLRLKQQAGSLSPDDLRE |
| | | | | | LAGYLKLDYEPERDFLPSDLRG |
| 1 | WP_081100188.1 | MULTISPECIES: | Pseudomonas | 474 | MRRRTPLIHTTFLASLCMCTTLL |
| | | TolC | putida group | | AEPQRAELLQVYQHAVEQDAR |
| | | family outer | | | LSAARHEFQALSERVPQARAGL |
| | | membrane | | | LPTLNAGATFESTRMQRDEPAL |
| | | protein | | | TRTRSGMVYQANLNQPLLRLD |
| | | | | | RWYALKAAEAGTAQAGLDLA |
| | | | | | AKEQSLILQTAEAYFDTLRALD |
| | | | | | ALAAAKAEAKALKRQQEQAQG |
| | | | | | RLANGASSITDVLDAQAAYDN |
| | | | | | ASANRQLAQRKVDDAFEVLYR |
| | | | | | LTRQDYAQIAGIAHQLPVQPPV |
| | | | | | PNDASTWVDQAVQSNLSLLSSG |
| | | | | | HAVDAAVQASRQRKAGHAPTL |
| | | | | | DAVASYRKGDNDRFGYSNPTD |
| | | | | | FGRNGYRDDIAQGTLGLELNIP |
| | | | | | LYAGGLTSSQVREAGERLAQSE |
| | | | | | DEHEDRRREVVLQTRNLHRAV |
| | | | | | NADVEQVQARQQSIRSSQASLK |
| | | | | | ANQVGRALGSRNTADVLNAER |
| | | | | | QLYRAVREYNDARYDYIIDSLK |
| | | | | | LKQAAGSLAPQDLLELSHYLSR |
| | | | | | DYDPDRDFLPPEARRPVAG |
| 1 | WP_038705693.1 | TolC family | Pseudomonas soli | 475 | MLRKTLLPLLFLAPLAQAVAAE |
| | | outer | | | RATLMSVYAQVVEHNSDMAA |
| | | membrane | | | ARENYLARREAVPLARARLLPQ |
| | | protein | | | VALDAEAGDVRSDRRQVEQRS |
| | | | | | GSLYRLSLDQPLFDLSRWFDFK |
| | | | | | AAQSENEQAELDFSAFQQQLIL |
| | | | | | DTASRYFDTLLAEDNLATAKAE |
| | | | | | LRAFDRQLQQTRLSYDAGLSDQ |
| | | | | | NDMLSAQASFDRASANLIDSQR |
| | | | | | RSEDAYQALMRLTGQAQPALA |
| | | | | | GIRHSLPVQAPVPARAEAWVEQ |
| | | | | | AMAQNLRLRASQAAVSQAGQT |
| | | | | | LRSSKADHLPTFNLAMGYAEG |
| | | | | | DSDLMDSSAHFGQRSGNQRDS |
| | | | | | SVMVQMKLPLFSGGGTSARVR |
| | | | | | QATHELARTEYGRTSLEREVLE |
| | | | | | GSRNAFRAVVSDVEQVRARRQ |
| | | | | | SIISSQGSLRATEAGYEVGSRNI |
| | | | | | VDILDAQRDLYNAVRDYNVSR |
| | | | | | YAYIVDSLRLKQQAGSLSPEDL |
| | | | | | RELAGYLKLDYEPERDFLPSDL |
| | | | | | RG |
| 1 | WP_173203287.1 | TolC family | Pseudomonas sp. | 476 | MLRRLSLALAVASAASGMAWA |
| | | outer | S1-A32-2 | | ADAPAGSKTGLVTVYNEAVKN |
| | | membrane | | | NADLAAARANYGAISEVVPQA |
| | | protein | | | RAGLLPNLSAGADMTNTDTQV |
| | | | | | DRSIGDLDSSRSGTSYRASLSQP |
| | | | | | LFRADRWFQYQAAKATNEQAS |
| | | | | | LALSATEQNLILQSAETYFAVLR |
| | | | | | AQDTLASTKAEEAAFKRQLDQ |
| | | | | | ANERFDVGLSDKTDVLEAQAG |
| | | | | | YDTSRANRIIAERNVQDAFQAL |
| | | | | | VTLTNNEYAAIEGIQHTLPILVP |
| | | | | | VPNDAKAWVDTAAQQNLNLQ |
| | | | | | SSNFAVDAAEETLRQRKSGHAP |
| | | | | | TLDAVAQYQKGDNDSLGFNNS |
| | | | | | DALSVYDGDAEQTTIGLQLNIPI |
| | | | | | YSGGLTSSQVREAYQTLTQTEQ |
| | | | | | ERESLRRQIVENTRNLHRAVNT |
| | | | | | DVETVQARKQSIISNQSALEATE |
| | | | | | IGYQVGTRNIVDVLDAQRQLYS |
| | | | | | SVRNYNDARYDYIINNLRLKQA |
| | | | | | AGTLAPSDLDALSSFLKPDYNP |
| | | | | | DQDFLPPDLAKAAEAQLNGNPE |
| | | | | | Y |
| 1 | WP_173171329.1 | TolC family | Pseudomonas sp. | 477 | MLRRLSLAVAVASAFAGSAWA |
| | | outer | TUM18999 | | AEAPLSAKTDLVTVYKDAVDN |
| | | membrane | | | NADLAAARANFEAVREAVPQA |
| | | protein | | | RSGLLPQINGGATANDTRTKLD |
| | | | | | EPSATSSRSGIAYQASLSQPIFRA |
| | | | | | DRWFQLQAAEASTEQASLEFSA |
| | | | | | TQQNLILQTAEGYFAVLRAQDN |
| | | | | | LASTKAEEAAFKRQLDQANERF |
| | | | | | DVGLSDKTDVLEAQAGFDTAR |
| | | | | | ANRIIAERNVDDAFQALVTLTN |
| | | | | | REYASIEGIQHTLPVVVPAPNDA |
| | | | | | KAWVDTAAQQNLNLQASNYA |
| | | | | | VDAAEETLRQRKAGHAPTLDA |
| | | | | | VAQYQKGDNDSLGFTNSAGSP |
| | | | | | VRYGSDVEQRTIGLQLNIPIYSG |
| | | | | | GLTSSQVRESYQRLNQTEQQRE |
| | | | | | SLRRQVVQNTRNYHRAVNTDV |
| | | | | | EQVKARKQSIISNQSALEATEIG |
| | | | | | YQVGTRNIVDVLDAQRQLYSS |
| | | | | | VRDYNNARYDYILDNLRLKQA |
| | | | | | AGTLSPSDLEALASFLKSDYDP |
| | | | | | DKDFLPPDLAKAAEASLKNKSN |
| | | | | | Y |
| 1 | WP_201419198.1 | TolC family | Pseudomonas | 478 | MLRKLSLVVAVSFASSGLTWA |
| | | outer | syringae | | AELPMPTQTGLLSVYQQAVDN |
| | | membrane | | | NADLAASRADYDARKEAVPQA |
| | | protein | | | RAGLLPNISGSAQNSSTRTSIDQ |
| | | | | | PRAVATRSGNLYQATLSQPIFR |
| | | | | | ADRWFQLQAAEASNEQAAMEL |
| | | | | | SATEQNLILQSAQSYFSVLRAQ |
| | | | | | DNLASTKAEEAAFKRQLDQAN |
| | | | | | ERFDVGLSDKTDVLQAQASYD |
| | | | | | TSRASRLIAQRQVDDAFQALVT |
| | | | | | LTNREYNSIEGIVHTLPVLAPTP |
| | | | | | NDAKAWVDTAAQQNLNLLAS |
| | | | | | NYAVNAAEETLRQRKAGHAPT |
| | | | | | LDAVATYQRGDNDALGFSNPN |
| | | | | | YTGRNYGGDVEQRSIGVQLNIPI |
| | | | | | YSGGLTSSQVREAYARLSQSEQ |
| | | | | | RRESLRRQVVENTRNLHRAVN |
| | | | | | TDVEQVQARKQSIISNQSALEA |
| | | | | | TEIGYQVGTRNIVDVLDAQRQL |
| | | | | | YASVRDYNNTRYDYILDNLRL |
| | | | | | KQAAGTLNPGDLQDLSRYLKA |
| | | | | | DYNPDKDFLPPDLATAAQKNFE |
| | | | | | RPAKR |
| 1 | WP_003377116.1 | MULTISPECIES: | Pseudomonas | 479 | MLRKLSLVVAVSLASSGLTWA |
| | | TolC | syringae group | | ADLPLPTKTGLLNVYQQAVDN |
| | | family outer | | | NADLAASRADYDARKEAVPQA |
| | | membrane | | | RAGLLPNISGSVQNTNTRTSIDR |
| | | protein | | | PSAVATRSGTVYQATLSQPIFRA |
| | | | | | DRWFQLQAAEAVNEQAALELS |
| | | | | | ATEQNLILQSAQSYFSVLRAQD |
| | | | | | NLASTKAEEAAFKRQLDQANE |
| | | | | | RFDVGLSDKTDVLQAQASYDT |
| | | | | | SRASRLIAKRQVDDAFQALVTL |
| | | | | | TNREYNSIEGIVHTLPVLAPTPN |
| | | | | | DAKAWVDTAAQQNLNLLASN |
| | | | | | YAVSAAEETLRQRKAGHAPTL |
| | | | | | DAVATYQRGDNDALGENNPNY |
| | | | | | TGQNYGGDVEQRSIGVQLNIPI |
| | | | | | YSGGLTSSQVREAYSRLSQSEQ |
| | | | | | RRESLRRQVVENTRNLHRAVN |
| | | | | | TDVEQVQARKQSIISNQSALEA |
| | | | | | TEIGYQVGTRNIVDVLDAQRQL |
| | | | | | YASVRDYNNTRYDYILDNLRL |
| | | | | | KQAAGTLNPGDLQDLSRYLKP |
| | | | | | DYNPDKDFLPPDLATAAQKNFE |
| | | | | | RPAQR |
| 1 | WP_004654543.1 | MULTISPECIES: | Pseudomonas | 480 | MLRKLSLVVAVSFASSGLTWA |
| | | TolC | syringae group | | ADMPLPTQTGLLNVYQQAVDN |
| | | family outer | | | NADLAASRADYDARKEAVPQA |
| | | membrane | | | RAGLLPNISGSAQSNNTRTSIDQ |
| | | protein | | | PRAVATRSGNVYQATLSQPIFR |
| | | | | | ADRWFQLQAAEASNEQAALEL |
| | | | | | SATEQNLILQAAQSYFSVLRAQ |
| | | | | | DNLASTKAEEAAFKRQLDQAN |
| | | | | | ERFDVGLSDKTDVLQAQASYD |
| | | | | | TSRASRILAKRQVDDAFQALVT |
| | | | | | LTNREYNSIEGIVHTLPVLAPTP |
| | | | | | NDAKAWVDTAAQQNLNLLAS |
| | | | | | NYAVSAAEETLRQRKAGHAPT |
| | | | | | LDAVATYQRGDNDALGFSNPN |
| | | | | | YTGRNYGGDVEQRSIGVQLNIPI |
| | | | | | YSGGLTSSQVREAYARLSQSEQ |
| | | | | | RRESLRRQVVENTRNLHRAVN |
| | | | | | TDVEQVQARKQSIISNQSALEA |
| | | | | | TEIGYQVGTRNIVDVLDAQRQL |
| | | | | | YASVRDYNNTRYDYILDNLRL |
| | | | | | KQAAGTLSPGDLQDLSRYLKA |
| | | | | | DYNPDKDFLPPDLATAAQKNFE |
| | | | | | RPAKR |
| 1 | WP_004665124.1 | MULTISPECIES: | Pseudomonas | 481 | MKHNFYLCALSLYLFALGAQE |
| | | TolC | syringae group | | ALAREPYSKRADLLAVYQAAS |
| | | family outer | | | LNDARLSAARHTYQAQLEATP |
| | | membrane | | | QARSGLLPSLTAGATTEVTQLD |
| | | protein | | | RDEPALSRERSSTTFRANLSQPL |
| | | | | | FRIDRWYQLKAAEFTVSQAALE |
| | | | | | LSAKEQELILTAAQAYFETLRQ |
| | | | | | LDARAAAEAEEAALLRQRDQA |
| | | | | | QGRLEDGASSITDVYDAQAAY |
| | | | | | DNARANRQLIQRKVDDAYEAL |
| | | | | | YRLTKNSYDSLDGMGHQLPVIP |
| | | | | | PNPNNPVLWVDAAIKQNLQLQ |
| | | | | | AAEHAVSAAEQTLRQRKAGHA |
| | | | | | PTVDAVVSYRTGNNDGFGYSN |
| | | | | | PAESGTGGYRGNVAQSSIGVEL |
| | | | | | SIPLYSGGMVNSQVRESTERLY |
| | | | | | QSQDEQEDRRREVVLNTRNAF |
| | | | | | RGINSDIEQIAARRQSIRSGRKS |
| | | | | | VEANKVGVDIGSRNVVDVLNA |
| | | | | | ERQLYAAVRDYNDARYDYILD |
| | | | | | NLKLKQAAGSLSPDDLRSLSAY |
| | | | | | LNHDYDPSRDFLPPDI |
| 1 | WP_007251036.1 | MULTISPECIES: | Pseudomonas | 482 | MLRKLSWVVAVSFASSGLTWA |
| | | TolC | syringae group | | ADLPMPIKTGLLNVYQQAVDN |
| | | family outer | | | NADLAASRADYDARKEVVPQA |
| | | membrane | | | RAGLLPNISGSVQNTDTRTSIDQ |
| | | protein | | | PRAVASRSGTVYQATLSQPIFR |
| | | | | | ADRWFQLQAAEAVNEQAALEL |
| | | | | | SATEQNLILQSAQSYFSVLRAQ |
| | | | | | DNLASTKAEEAAFKRQLDQAR |
| | | | | | ERFDVGLSDKTDVLQAQASYD |
| | | | | | TSRASRLLAQRQVDDAFQALVT |
| | | | | | LTNREYNSIEGIVHTLPVLAPTP |
| | | | | | NDAKAWVDTAAQQNLNLLAS |
| | | | | | NYAVSAAEETLRQRKAGHAPT |
| | | | | | LDAVATYQRGDNDALGENNPN |
| | | | | | YTGRNYSGDVEQRSIGVQLNIPI |
| | | | | | YSGGLTSSQVREAYSRLSQSEQ |
| | | | | | RRESLRRQVVENTRNLHRAVN |
| | | | | | TDVEQVQARKQSIISNQSALEA |
| | | | | | TEIGYQVGTRNIVDVLDAQRQL |
| | | | | | YASVRDYNNTRYDYILDNLRL |
| | | | | | KQAAGTLNPGDLQDLSRYLKP |
| | | | | | DYNPDKDFLPPDLATAAQKNFE |
| | | | | | RPAQR |
| 1 | WP_010193662.1 | MULTISPECIES: | Pseudomonas | 483 | MLRKLSLVVAVSFASSGLTWA |
| | | TolC | syringae group | | ADMPLPTQTGLLNVYQQAVDN |
| | | family outer | genomosp. 2 | | NADLAASRADYDARKEAVPQA |
| | | membrane | | | RAGLLPNISGSAQSNNTRTSIDQ |
| | | protein | | | PRAVATRSGNVYQATLSQPIFR |
| | | | | | ADRWFQLQAAEASNEQAALEL |
| | | | | | SASEQNLILQAAQSYFSVLRAQ |
| | | | | | DNLASTKAEEAAFKRQLDQAN |
| | | | | | ERFDVGLSDKTDVLQAQASYD |
| | | | | | TSRASRILAKRQVDDAFQALVT |
| | | | | | LTNREYNSIEGIVHTLPVLAPTP |
| | | | | | NDAKAWVDTAAQQNLNLLAS |
| | | | | | NYAVSAAEETLRQRKAGHAPT |
| | | | | | LDAVATYQRGDNDALGFSNPN |
| | | | | | YTGRNYGGDVEQRSIGVQLNIPI |
| | | | | | YSGGLTSSQVREAYARLSQSEQ |
| | | | | | RRESLRRQVVENTRNLHRAVN |
| | | | | | TDVEQVQARKQSIISNQSALEA |
| | | | | | TEIGYQVGTRNIVDVLDAQRQL |
| | | | | | YASVRDYNNTRYDYILDNLRL |
| | | | | | KQAAGTLSPGDLQDLSRYLKA |
| | | | | | DYNPDKDFLPPDLATAAQKNFE |
| | | | | | RPAKR |
| 1 | WP_007251649.1 | TolC family | Pseudomonas | 484 | MKPFSSLCLVMLACTFACSETV |
| | | outer | syringae group | | FAREQPEQHADLLGVYQAAAL |
| | | membrane | genomosp. 3 | | NDAQLSAARHAYNAQREAVPQ |
| | | protein | | | ARAGLLPNLTAGATSEVTSLKR |
| | | | | | DEPSLTRERSGITFRANVSQPLF |
| | | | | | RIDRWFQLKAAQSSVSQAELEL |
| | | | | | SAKEQNLLLTAAQAYFETLRQL |
| | | | | | DAYAAAQAEEAALLRQRDQAQ |
| | | | | | GRLEDGASSITDVYDAQAAYD |
| | | | | | NARANRQLVQRKVDDAYEALE |
| | | | | | RLTKHTYTSIAGMRHQMPVEAP |
| | | | | | IPNDARAWVDVTVRQNLQLQA |
| | | | | | SEHAVSAAEQTLSQRKAGHAPT |
| | | | | | IDAVASYRKGDNDSFGYSNPTD |
| | | | | | FGSNGYRDNVAQSSIGIELNIPL |
| | | | | | YSGGMTNSQIKESTERLYQSQD |
| | | | | | EQEDRRREVVLTTRNAFRGINA |
| | | | | | GIEQIAARRQSIVSGQKSVEANQ |
| | | | | | VGVDVGSRNIADVLNAQRQLY |
| | | | | | AAVRDYNNARYDYILDNLKLK |
| | | | | | QAAGTLSPDDLKALASYLKQD |
| | | | | | YDPARDFLPPGV |
| 1 | WP_063033408.1 | TolC family | Pseudomonas | 485 | MLRKLSLALAVSCATNGLVWA |
| | | outer | yamanorum | | AEAPLTTKTDLVSVYQEAVDN |
| | | membrane | | | NADLAAARAQYGAQKEVVPQ |
| | | protein | | | ARAGLLPNLSAGADINNVRTQL |
| | | | | | DQPAATVNRDAHSWRATLSQP |
| | | | | | LFRADRWFQLQAAEAVNEQAA |
| | | | | | LQLSATEQNLILQSAENYFAVL |
| | | | | | RAQDNLASTKAEEAAFKRQLD |
| | | | | | QSNERFDVGLSDKTDVLQSQAS |
| | | | | | YDTARANRILAQRQVDDAFEA |
| | | | | | LITLTNRQYNSIQGIVHTLPVLPP |
| | | | | | SPNDAKAWVETAGRQNLNLLA |
| | | | | | SNYAVTAAEETLKQRKAGHAP |
| | | | | | TLDAVAQYEKGDNDALGFSNP |
| | | | | | SAFGQPYHGDVSQRTVGLQLRI |
| | | | | | PLYSGGLTSSQVRESYSRLGQT |
| | | | | | EQQREGLRRQVVENTRNLHRA |
| | | | | | VNTDVEQVQARRQSIISNQSAV |
| | | | | | EATEIGYQVGTRNIVDVLDAQR |
| | | | | | QLYTSVRNYNNSRYDYILDNLR |
| | | | | | LKQAAGTLNPGDLQDLARYLK |
| | | | | | ADYNPDKDFLPPDLAKAAAEQ |
| | | | | | LKANPGY |
| 1 | WP_015464123.1 | TolC family | Psychromonas sp. | 486 | MKIKQLTLATFIALTSFNSVVSA |
| | | outer | CNPT3 | | DDLLQIYETAQLKDPIIQKSKAQ |
| | | membrane | | | YDLYLERVSASNASLLPQIGFGL |
| | | protein | | | NAGKSKDKDDINSNTNYGANL |
| | | | | | SLSQSLYNGTYWKELDLAEIQA |
| | | | | | TQYAAIYGYAQQNLILRTSSIYF |
| | | | | | NVLRAHQSVQSVQANKRAVAR |
| | | | | | QLEQTKQRFDVGLIAITDVHEA |
| | | | | | QAEFDRTNADLIKAKNQLINSY |
| | | | | | YALRELTGEDIRNIDLLNTDIFSP |
| | | | | | APLEGNVTLWHNNALEHNLLL |
| | | | | | HEKRIAKDLAQMQIKLARTGH |
| | | | | | QPTLDLSARIGYNNTDYDALRR |
| | | | | | SDIDMSDASIGIEFNLPIYTGGKT |
| | | | | | NSLVNQATYSYVMASEDLIQSF |
| | | | | | RSIDADIKSGYNNVRASISSITA |
| | | | | | YEQTVISSKSALEAIEAGFEVGT |
| | | | | | RTIVDVLDSTRRLFESEDLLANA |
| | | | | | RYDYILSRLQLKYSAGTLSIQDI |
| | | | | | KDISAGLIKASDK |
| 1 | WP_013573963.1 | MULTISPECIES: | Rahnella | 487 | MKKLLPLLIGLSLGGFSTISQAE |
| | | outer | | | NLMQVYQQARTSNPDLRKSAA |
| | | membrane | | | DRDAAFEKINESRSPLLPQLGLG |
| | | channel | | | ADYNYTNGYRDANGVNSDVGS |
| | | protein TolC | | | ASLQLTQTIFDMSKWRALTLQE |
| | | | | | KTAGIQDVSYQTDQQSLILNSA |
| | | | | | TAYFNVLSAIDSLSYTEAQKQAI |
| | | | | | YRQLDQTTQRFNVGLVAITDVQ |
| | | | | | NARSQYDTVLANEVTARNNLD |
| | | | | | NALESLRQVTGMYYPQLSSLNV |
| | | | | | DNFNTTRPQPVATLLKEAESRN |
| | | | | | LSLLSARLSQDLAREQIRYAETG |
| | | | | | HMPTVDLTASTGISNTKYHGDK |
| | | | | | TGGSSSYQDADAGQNKIGLSFS |
| | | | | | LPLYSGGSVTSQVKQAQYNFV |
| | | | | | GASEQLESAHRSVVQTVRSSFN |
| | | | | | NISASISSINAYKQAVVSAQSSL |
| | | | | | DAMEAGYQVGTRTIVDVLDAT |
| | | | | | TTLYNAKQQLSSARYTYLINQL |
| | | | | | NIKSALGTLNESDLLALNGALG |
| | | | | | KPVPTAPDQVAPETPDQDAAA |
| | | | | | DGYGNKTAAAPAPRVQQASAS |
| | | | | | TATTTPKKSGNPFQN |
| 1 | WP_032690679.1 | MULTISPECIES: | Raoultella | 488 | MKKLFPILIGLGLTGFSAMSQAE |
| | | outer | | | NLMQVYQQARVSNPDLRKSAA |
| | | membrane | | | DRDAAFEKINEARSPLLPQLGL |
| | | channel | | | GADYTYTNGYRDSNGVNSNVT |
| | | protein TolC | | | SGSLQLTQTLFDMSKWRALTLQ |
| | | | | | EKAAGIQDVTYQTDQQTLILNT |
| | | | | | ATAYFNVLSAIDTLSYTEAQKQ |
| | | | | | AIYRQLDQTTQRFNVGLVAITD |
| | | | | | VQNARSQYDSVLANEVTARNN |
| | | | | | LDNAVEALRQVIGNYYPELAS |
| | | | | | LNVDGFKTNKPQTVNALLKEA |
| | | | | | ENRNLSLLQARLNQDLAREQIR |
| | | | | | QAQDGHLPTLDLTASTGVSNTS |
| | | | | | YSGSKTHNSTQYNDNDAGQNK |
| | | | | | IGLSFSLPLYQGGMVNSQVKQA |
| | | | | | QYNFVGASEQLESSHRSVVQTV |
| | | | | | RSSFNNINASISSINAYKQAVVS |
| | | | | | AQSSLDAMEAGYSVGTRTIVDV |
| | | | | | LDATTTLYNAKQQLSSARYNY |
| | | | | | LINQLNIKSALGTLNEQDLVAL |
| | | | | | NNTLGKPVSTSPDSVAPETPQQ |
| | | | | | DANADGYSSNAAPAATPASTRT |
| | | | | | TKTSGANPFRQ |
| 1 | WP_004867776.1 | outer | Raoultella | 489 | MKKLFPILIGLGLTGFSAMSQAE |
| | | membrane | ornithinolytica | | NLMQVYQQARVSNPDLRKSAA |
| | | channel | | | DRDAAFEKINEARSPLLPQLGL |
| | | protein TolC | | | GADYTYTNGYRDSNGVNSNVT |
| | | | | | SGSLQLTQTLFDMSKWRALTLQ |
| | | | | | EKAAGIQDVTYQTDQQTLILNT |
| | | | | | ATAYFNVLSAIDTLSYTEAQKQ |
| | | | | | AIYRQLDQTTQRFNVGLVAITD |
| | | | | | VQNARSQYDSVLANEVTARNN |
| | | | | | LDNAVEALRQVIGNYYPELAS |
| | | | | | LNVDGFKTNKPQTVNALLKEA |
| | | | | | ENRNLSLLQARLNQDLAREQIR |
| | | | | | QAQDGHLPTLDLTASTGISNTT |
| | | | | | YNGSKTNDPTRYGDTDAGQNK |
| | | | | | IGLSFSLPLYQGGMVNSQVKQA |
| | | | | | QYNFVGASEQLESSHRSVVQTV |
| | | | | | RSSFNNINASISSINAYKQAVVS |
| | | | | | AQSSLDAMEAGYSVGTRTIVDV |
| | | | | | LDATTTLYNAKQQLSSARYNY |
| | | | | | LINQLNIKSALGTLNEQDLLALN |
| | | | | | NTLGKPVSTSPDSVAPETPQQD |
| | | | | | ANADGYSSNAAPAATPASTRTT |
| | | | | | KTSGANPFRQ |
| 1 | WP_001575014.1 | MULTISPECIES: | Salmonella | 490 | MQMKKLLPILIGLSLSGFSTLSQ |
| | | outer | | | AENLMQVYQQARLSNPELRKS |
| | | membrane | | | AADRDAAFEKINEARSPLLPQL |
| | | channel | | | GLGADYTYSNGYRDANGINSN |
| | | protein TolC | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVTGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSKTNAAQYDDSNMGQN |
| | | | | | KIGLNFSLPLYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYDAKQQLANARY |
| | | | | | TYLINQLNIKYALGTLNEQDLL |
| | | | | | ALNSTLGKPIPTSPESVAPETPD |
| | | | | | QDAAADGYNAHSAAPAVQPTA |
| | | | | | ARANSNNGNPFRH |
| 1 | WP_001663008.1 | MULTISPECIES: | Salmonella | 491 | MQMKKLLPILIGLSLSGFSTLSQ |
| | | outer | | | AENLMQVYQQARLSNPELRKS |
| | | membrane | | | AADRDAAFEKINEARSPLLPQL |
| | | channel | | | GLGADYTYSNGYRDANGINSN |
| | | protein TolC | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVTGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSKTNSAQYDDSNMGQN |
| | | | | | KIGLNFSLPLYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYDAKQQLANARY |
| | | | | | TYLINQLNIKYALGTLNEQDLL |
| | | | | | ALNSTLGKPIPTSPESVAPETPE |
| | | | | | QDAAADGYNAHSAAPAVQPTA |
| | | | | | ARANSNNGNPFRH |
| 1 | WP_015899378.1 | MULTISPECIES: | Salmonella | 492 | MQMKKLLPILIGLSLSGFSTLSQ |
| | | outer | | | AENLMQVYQQARLSNPELRKS |
| | | membrane | | | AADRDAAFEKINEARSPLLPQL |
| | | channel | | | GLGADYTYSNGYRDANGINSN |
| | | protein TolC | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVTGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSKTNSAQYDDSNMGQN |
| | | | | | KIGLNFSLPLYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYDAKQQLANARY |
| | | | | | TYLINQLNIKYALGTLNEQDLL |
| | | | | | ALNSTLGKPIPTSPESVAPETPD |
| | | | | | QDAAADGYNAHSAAPAVQPTA |
| | | | | | ARANSNNGNPFRH |
| 1 | WP_072100461.1 | MULTISPECIES: | Salmonella | 493 | MQMKKLLPILIGLSLSGFSTLSQ |
| | | outer | | | AENLMQVYQQARLSNPELRKS |
| | | membrane | | | AADRDAAFEKINEARSPLLPQL |
| | | channel | | | GLGADYTYSNGYRDANGINSN |
| | | protein TolC | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVIGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSKTNSTQYDDSNMGQN |
| | | | | | KIGLNFSLPLYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYDAKQQLANARY |
| | | | | | TYLINQLNIKYALGTLNEQDLL |
| | | | | | ALNSTLGKPIPTSPESVAPETPE |
| | | | | | QDAAADGYNAHSAAPAVQPTA |
| | | | | | ARANSNNGNPFRH |
| 1 | WP_072100718.1 | MULTISPECIES: | Salmonella | 494 | MQMKKLLPILIGLSLSGFSTLSQ |
| | | outer | | | AENLMQVYQQARLSNPELRKS |
| | | membrane | | | AADRDAAFEKINEARSPLLPQL |
| | | channel | | | GLGADYTYSNGYRDANGINSN |
| | | protein TolC | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVTGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSKTNSAQYDDSNMGQN |
| | | | | | KIGLNFSLPLYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYDAKQQLANARY |
| | | | | | TYLINQLNIKYALGTLNEQDLL |
| | | | | | ALNSTLGKPIPTSPESVAPETPD |
| | | | | | QDAAADGYNAHSAAPAVQPTA |
| | | | | | ARANSNNGNPFRHR |
| 1 | WP_072103650.1 | MULTISPECIES: | Salmonella | 495 | MQMKKLLPILIGLSLSGFSTLSQ |
| | | outer | | | AENLMQVYQQARLSNPELRKS |
| | | membrane | | | AADRDAAFEKINEARSPLLPQL |
| | | channel | | | GLGADYTYSNGYRDANGINSN |
| | | protein TolC | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVIGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSKTNSAQYDDSNMGQN |
| | | | | | KIGLNFSLPLYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYDAKQQLANARY |
| | | | | | TYLINQLNIKYALGTLNEQDLL |
| | | | | | ALNSTLGKPIPTSPETPEQDAAA |
| | | | | | DGYNAHSAAPAVQPTAARANS |
| | | | | | NNGNPFRH |
| 1 | WP_072162109.1 | MULTISPECIES: | Salmonella | 496 | MQMKKLLPILIGLSLSGFSTLSQ |
| | | outer | | | AENLMQVYQQARLSNPELRKS |
| | | membrane | | | AADRDAAFEKINEARSPLLPQL |
| | | channel | | | GLGADYTYSNGYRDANGINSN |
| | | protein TolC | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVIGNYYPE |
| | | | | | LASLNVEHFKTDKPKAINALLK |
| | | | | | EAENRNLSLLQARLSQDLAREQ |
| | | | | | IRQAQDGHLPTLNLTASTGISDT |
| | | | | | SYSGSKTNSTQYDDSNMGQNKI |
| | | | | | GLNFSLPLYQGGMVNSQVKQA |
| | | | | | QYNFVGASEQLESAHRSVVQT |
| | | | | | VRSSFNNINASISSINAYKQAVV |
| | | | | | SAQSSLDAMEAGYSVGTRTIVD |
| | | | | | VLDATTTLYDAKQQLANARYT |
| | | | | | YLINQLNIKYALGTLNEQDLLA |
| | | | | | LNSTLGKPIPTSPESVAPETPEQ |
| | | | | | DAAADGYNAHSAAPAVQPTAA |
| | | | | | RANSNNGNPFRH |
| 1 | WP_015703015.1 | outer | Salmonella | 497 | MQMKKLLPILIGLSLSGFSTLSQ |
| | | membrane | bongori | | AENLMQVYQQARLSNPELRKS |
| | | channel | | | AADRDAAFEKINEARSPLLPQL |
| | | protein TolC | | | GLGADYTYSNGYRDANGINSN |
| | | | | | ETSASLQLTQTLFDMSKWRALT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVIGNYYPE |
| | | | | | LASLNVEHFKTDKPKAVNALL |
| | | | | | KEAENRNLSLLQARLSQDLARE |
| | | | | | QIRQAQDGHLPTLNLTASTGVS |
| | | | | | DTSYSGSNTHGPGSQYDDSNM |
| | | | | | GQNKIGLNFSLPIYQGGMVNSQ |
| | | | | | VKQAQYNFVGASEQLESAHRS |
| | | | | | VVQTVRSSFNNINASISSINAYK |
| | | | | | QAVVSAQSSLDAMEAGYSVGT |
| | | | | | RTIVDVLDATTTLYDAKQQLAN |
| | | | | | ARYTYLINQLNIKYALGTLNEQ |
| | | | | | DLLALNSTLGKPIPTSPESVAPE |
| | | | | | TPEQDAAADGYSANTAAPAVQ |
| | | | | | PTAARTTSSKSGNPFHN |
| 1 | WP_022742825.1 | outer | Salmonella | 498 | MQMKKLLPILIGLSLSGFSTLSQ |
| | | membrane | enterica | | AENLMQVYQQARLSNPELRKS |
| | | channel | | | AADRDAAFEKINEARSPLLPQL |
| | | protein TolC | | | GLGADYTYSNGYSDANGINSNE |
| | | | | | TSASLQLTQTLFDMSKWRGLTL |
| | | | | | QEKAAGIQDVTYQTDQQTLILN |
| | | | | | TANAYFKVLNAIDVLSYTQAQ |
| | | | | | KEAIYRQLDQTTQRFNVGLVAI |
| | | | | | TDVQNARAQYDTVLANEVTAR |
| | | | | | NNLDNAVEELRQVIGNYYPEL |
| | | | | | ASLNVEHFKTDKPKAVNALLK |
| | | | | | EAENRNLSLLQARLSQDLAREQ |
| | | | | | IRQAQDGHLPTLNLTASTGISDT |
| | | | | | SYSGSKTNSTQYDDSNMGQNKI |
| | | | | | GLNFSLPLYQGGMVNSQVKQA |
| | | | | | QYNFVGASEQLESAHRSVVQT |
| | | | | | VRSSFNNINASISSINAYKQAVV |
| | | | | | SAQSSLDAMEAGYSVGTRTIVD |
| | | | | | VLDATTTLYDAKQQLANARYT |
| | | | | | YLINQLNIKYALGTLNEQDLLA |
| | | | | | LNSTLGKPIPTSPESVAPETPDQ |
| | | | | | DAAADGYNAHSAAPAVQPTAA |
| | | | | | RANSNNGNPFRH |
| 1 | WP_072100990.1 | outer | Salmonella | 499 | MQMKKLLPILIGLSLSGFSTLSQ |
| | | membrane | enterica | | AENLMQVYQQARLSNPELRKS |
| | | channel | | | AADRDAAFEKINEARSPLLPQL |
| | | protein TolC | | | GLGADYTYSNGYRDANGINSN |
| | | | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVTGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSKTNAAQYDDSNMGQN |
| | | | | | KIGLNFSLPLYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYDAKQQLANARY |
| | | | | | TYLINQLNIKYALGTLNEQDLL |
| | | | | | ALNSTLGKPIPTSPESVAPETPE |
| | | | | | QDAAADGYNAHSAAPAVQPTA |
| | | | | | ARANSNNGNPFRH |
| 1 | WP_072101802.1 | outer | Salmonella | 500 | MQMKKLLPILIGLSLSGFSTLSQ |
| | | membrane | enterica | | AENLMQVYQQARLSNPELRKS |
| | | channel | | | AADRDAAFEKINEARSPLLPQL |
| | | protein TolC | | | GLGADYTYSNGYRDANGINSN |
| | | | | | ETSASLQLTQTLFDMSKWRGLT |
| | | | | | LQEKAAGIQDITYQTDQQTLILN |
| | | | | | TANAYFKVLNAIDVLSYTQAQ |
| | | | | | KEAIYRQLDQTTQRFNVGLVAI |
| | | | | | TDVQNARAQYDTVLANEVTAR |
| | | | | | NNLDNAVEELRQVTGNYYPEL |
| | | | | | ASLNVEHFKTDKPKAVNALLK |
| | | | | | EAENRNLSLLQARLSQDLAREQ |
| | | | | | IRQAQDGHLPTLNLTASTGISDT |
| | | | | | SYSGSKTNSTQYDDSNMGQNKI |
| | | | | | GLNFSLPLYQGGMVNSQVKQA |
| | | | | | QYNFVGASEQLESAHRSVVQT |
| | | | | | VRSSFNNINASISSINAYKQAVV |
| | | | | | SAQSSLDAMEAGYSVGTRTIVD |
| | | | | | VLDATTTLYDAKQQLANARYT |
| | | | | | YLINQLNIKYALGTLNEQDLLA |
| | | | | | LNSTLGKPIPTSPESVAPETPDQ |
| | | | | | DAAADGYNAHSAAPAVQPTAA |
| | | | | | RANSNNGNPFRH |
| 1 | WP_072156293.1 | outer | Salmonella | 501 | MQMKKLLPILIGLSLSGFSTLSQ |
| | | membrane | enterica | | AENLMQVYQQARLSNPELRKS |
| | | channel | | | AADRDAAFEKINEARSPLLPQL |
| | | protein TolC | | | GLGADYTYSNGYRDANGINSN |
| | | | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDSVLANEVTA |
| | | | | | RNNLDNAVEELRQVTGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSNTHGPGSQYDDSNMG |
| | | | | | QNKIGLNFSLPLYQGGMVNSQV |
| | | | | | KQAQYNFVGASEQLESAHRSV |
| | | | | | VQTVRSSFNNINASISSINAYKQ |
| | | | | | AVVSAQSSLDAMEAGYSVGTR |
| | | | | | TIVDVLDATTTLYDAKQQLAN |
| | | | | | ARYTYLINQLNIKYALGTLNEQ |
| | | | | | DLLALNSTLGKPIPTSPENVAPE |
| | | | | | TPEQDAAADGYNAHSAAPAVQ |
| | | | | | PTAARANSNNGNPFRH |
| 1 | WP_072200835.1 | outer | Salmonella | 502 | MQMKKLLPILIGLSLSGFSTLSQ |
| | | membrane | enterica | | AENLMQVYQQARLSNPELRKS |
| | | channel | | | AADRDAAFEKINEARSPLLPQL |
| | | protein TolC | | | GLGADYTYSNGYRDANGINSN |
| | | | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVTGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSKTNAAQYDDSNMGQN |
| | | | | | KIGLNFSLPLYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYDAKQQLANARY |
| | | | | | TYLINQLNIKYALGTLNEQDLL |
| | | | | | ALNSKLGKPIPTSPESVAPETPD |
| | | | | | QDAAADGYNAHSAAPAVQPTA |
| | | | | | ARANSNNGNPFRH |
| 1 | WP_072203379.1 | outer | Salmonella | 503 | MQMKKLLPILIGLSLSGFSTLSQ |
| | | membrane | enterica | | AENLMQVYQQARLSNPELRKS |
| | | channel | | | AADRDAAFEKINEARSPLLPQL |
| | | protein TolC | | | GLGADYTYSNGYRDANGINSN |
| | | | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVIGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSKTNSAQYDDSNMGQN |
| | | | | | KIGLNFSLPLYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYDAKQQLANARY |
| | | | | | TYLINQLNIKYALGTLNEQDLL |
| | | | | | ALNSTLGKPIPTSPESIAPETPDQ |
| | | | | | DAAADGYNAHSAAPAVQPTAA |
| | | | | | RANSNNGNPFRH |
| 1 | WP_074416937.1 | outer | Salmonella | 504 | MQMKKLLPILIGLSLSGFSTLSQ |
| | | membrane | enterica | | AENLMQVYQQARLSNPELRKS |
| | | channel | | | AADRDAAFEKINEARSPLLPQL |
| | | protein TolC | | | GLGADYTYSNGYRDANGINSN |
| | | | | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVIGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSNTHGPGSQYDDSNMG |
| | | | | | QNKIGLNFSLPLYQGGMVNSQV |
| | | | | | KQAQYNFVGASEQLESAHRSV |
| | | | | | VQTVRSSFNNINASISSINAYKQ |
| | | | | | AVVSAQSSLDAMEAGYSVGTR |
| | | | | | TIVDVLDATTTLYDAKQQLAN |
| | | | | | ARYTYLINQLNIKYALGTLNEQ |
| | | | | | DLLALNSTLGKPIPTSPESVAPE |
| | | | | | TPDQDAAADGYNAHSAAPAVQ |
| | | | | | PTAARANSNNGNPFRH |
| 1 | NP_462101.3 | outer | Salmonella | 505 | MQMKKLLPILIGLSLSGFSTLSQ |
| | | membrane | enterica subsp. | | AENLMQVYQQARLSNPELRKS |
| | | channel | enterica serovar | | AADRDAAFEKINEARSPLLPQL |
| | | | Typhimurium str. | | GLGADYTYSNGYRDANGINSN |
| | | | LT2 | | ETSASLQLTQTLFDMSKWRGLT |
| | | | | | LQEKAAGIQDVTYQTDQQTLIL |
| | | | | | NTANAYFKVLNAIDVLSYTQA |
| | | | | | QKEAIYRQLDQTTQRFNVGLVA |
| | | | | | ITDVQNARAQYDTVLANEVTA |
| | | | | | RNNLDNAVEELRQVIGNYYPE |
| | | | | | LASLNVEHFKTDKPKAVNALL |
| | | | | | KEAENRNLSLLQARLSQDLARE |
| | | | | | QIRQAQDGHLPTLNLTASTGISD |
| | | | | | TSYSGSKTNSTQYDDSNMGQN |
| | | | | | KIGLNFSLPLYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYDAKQQLANARY |
| | | | | | TYLINQLNIKYALGTLNEQDLL |
| | | | | | ALNSTLGKPIPTSPESVAPETPD |
| | | | | | QDAAADGYNAHSAAPAVQPTA |
| | | | | | ARANSNNGNPFRH |
| 1 | WP_125353317.1 | outer | Scandinavium | 506 | MKKLLPILIGLSLTGFSAVSHAE |
| | | membrane | goeteborgense | | NLLQVYQQARLSNPDLRKSAA |
| | | channel | | | DRDAAFEKINEARSPLLPQLGL |
| | | protein TolC | | | GADYTYTNGFRDNNGIDSNETS |
| | | | | | AALTLTQTIFDMSKWRALTLQE |
| | | | | | KSAGIQDVTYQTDQQTLILNTA |
| | | | | | TAYFNVLSAIDSLSFTEANKQA |
| | | | | | VYRQLDQTTQRFNVGLVAITDV |
| | | | | | QNARSQYDTVLANEVTARNNL |
| | | | | | DNAVEQLRQVIGNYYPELSSLN |
| | | | | | VSTLKMDKPQPVNALLKEAEG |
| | | | | | RNLALLQARLSQDLAREQIRYA |
| | | | | | ETGHMPTVGLTASTGVSDTSYD |
| | | | | | GSNATSSQYTDRNIGQNQIGLN |
| | | | | | FNLPLYSGGSVTSQVKQAQYNF |
| | | | | | VGASEQLESAHRNVVQTVRSSF |
| | | | | | NNINASISSINAYKQAVVSAQSS |
| | | | | | LDAMEAGYQVGTRTIVDVLDA |
| | | | | | TTTLYNAKQQLSSARYSYLINQ |
| | | | | | LNVKSALGTLNENDLLALNGTL |
| | | | | | GKAVSTSPEAVAPENAQQDAN |
| | | | | | ADGYNGASAAPAAQPAAQRTN |
| | | | | | TTSSNSSNPFKN |
| 1 | WP_004937240.1 | MULTISPECIES: | Serratia | 507 | MKKLLPLLIGLSLGGFSAMSQA |
| | | multidrug | | | ENLLQVYKQARESNPDLRKSA |
| | | efflux | | | ADRDAAFEKINEARSPLLPQLG |
| | | transporter | | | LTAGYDYTNGYRDSNGVNSNV |
| | | outer | | | TSGSLALTQTIFDMSKWRQLTL |
| | | membrane | | | QEKTAGISDVTFQTAEQQLILNT |
| | | subunit HasF | | | ATAYFNVLKAIDTLSYTQAQKD |
| | | | | | AVYRTLDQTTQRFNVGLVAITD |
| | | | | | VQNARSNYDTVLAAEVSARND |
| | | | | | LDNALETLRQVTGTFYPELASL |
| | | | | | NTDRFSTQRPEAVNNLLKEAEA |
| | | | | | RNLSLLSARLSQDLAREQIRAA |
| | | | | | QTGYMPTIDVSASTGISNTKYN |
| | | | | | GSNTGGANAARYSDSDAGQNK |
| | | | | | VGISFNLPLYSGGATNSQVKQA |
| | | | | | QYGFVGASEQLESSHRSVVQTV |
| | | | | | RSSFNNVNASISSINAYKQAVIS |
| | | | | | AQSSLDAMEAGYQVGTRTIVD |
| | | | | | VLDATTTLYNAKRQLSDARYT |
| | | | | | YLINQLNIKSALGTLNQNDLLLL |
| | | | | | NGALGKPVSTAPDAVAPQNRA |
| | | | | | QDAYADGYQDNAPMQQTAAP |
| | | | | | VPAATRASAPAVTTSQPARNSG |
| | | | | | NPFRN |
| 1 | WP_019453066.1 | MULTISPECIES: | Serratia | 508 | MKKLLPLLIGLSLGGFSAMSQA |
| | | outer | | | ENLLQVYKQARESNPDLRKSA |
| | | membrane | | | ADRDAAFEKINEARSPLLPQLG |
| | | channel | | | LSAGYNYNNGYRDSNGVNSNV |
| | | protein TolC | | | TSASLGLTQTIFDMSKWRALTL |
| | | | | | QEKTAGISDVTFQTAEQQLILNT |
| | | | | | ATAYFNVLSAIDTLSYTQANKQ |
| | | | | | AVYRTLDQTTQRFNVGLVAITD |
| | | | | | VQNARSSYDTVLAAEVTARNN |
| | | | | | LDNALEALRQITGTFYPELASLN |
| | | | | | TDRFSTQRPEAVNNLLKEAEAR |
| | | | | | NLSLLSARLSQDLAREQIRSAQT |
| | | | | | GYMPTVNLNASTGITNTRYNGS |
| | | | | | ATHDTLSSSRYSNSDAGQNQVG |
| | | | | | VSISLPLYSGGATNSQVEQAQY |
| | | | | | GFVGASEQLESAHRGVVQTVRS |
| | | | | | SFNNVNASISSINAYKQAVISAQ |
| | | | | | SSLDAMEAGYQVGTRTIVDVL |
| | | | | | NATSTLYNAKQQLSSARYTYLI |
| | | | | | NQLNIKSALGTLNQNDLLTLNG |
| | | | | | ALGKPVSTAPDAVAPQNRAQD |
| | | | | | AYADGYQDNAPMQQVAAPAP |
| | | | | | TATRAAAPAVSQPVRNSGNPFR |
| | | | | | N |
| 1 | WP_033654823.1 | MULTISPECIES: | Serratia | 509 | MKKLLPLLIGLSLGGFSAMSQA |
| | | multidrug | | | ENLLQVYKQARESNPDLRKSA |
| | | efflux | | | ADRDAAFEKINEARSPLLPQLG |
| | | transporter | | | LTAGYDYTNGYRDSNGVNSNV |
| | | outer | | | TSGSLALTQTIFDMSKWRQLTL |
| | | membrane | | | QEKTAGISDVTFQTAEQSLILNT |
| | | subunit HasF | | | ATAYFNVLKAIDTLSYTQAQKD |
| | | | | | AVYRTLDQTTQRFNVGLVAITD |
| | | | | | VQNARSNYDTVLAAEVSARND |
| | | | | | LDNALETLRQVTGAFYPELASL |
| | | | | | NTDRFSTQRPEAVNNLLKEAEA |
| | | | | | RNLSLLSARLSQDLAREQIRAA |
| | | | | | QTGYMPTIDVSASTGISNTKYN |
| | | | | | GSNTGGANAARYSDSDAGQNK |
| | | | | | VGISFNLPLYSGGATNSQVKQA |
| | | | | | QYGFVGASEQLESSHRSVVQTV |
| | | | | | RSSFNNVNASISSINAYKQAVIS |
| | | | | | AQSSLDAMEAGYQVGTRTIVD |
| | | | | | VLDATTTLYNAKRQLSDARYT |
| | | | | | YLINQLNIKSALGTLNQNDLLLL |
| | | | | | NGALGKPVSTAPDAVAPQNRA |
| | | | | | QDAYADGYQDNAPMQQTAAP |
| | | | | | VPAATRASAPAVTTSQPARNSG |
| | | | | | NPFRN |
| 1 | WP_006320591.1 | outer | Serratia inhibens | 510 | MKKLLPLLIGLSLSGFSAMSQA |
| | | membrane | | | ENLLQVYKQARESNPDLRKTA |
| | | channel | | | ADRDAAFEKINEARSPLLPQLG |
| | | protein TolC | | | LSAGYNRSYGYRDSRDMNSNT |
| | | | | | TSGSLALTQTIFDMSKWRALTL |
| | | | | | QEKTAGISDVTFQTSSQQLILDT |
| | | | | | ATAYFNVLSAIDSLSYTQAQKQ |
| | | | | | AVYRTLDQTTQRFNVGLVAITD |
| | | | | | VQNARSNYDTVLASEVTARNT |
| | | | | | LDNALEKLRQVTGTFYPELASL |
| | | | | | NTDSFNTKRPEAVNNLLKEAEA |
| | | | | | RNLSLLSARLSQDLAREQIKSA |
| | | | | | QTGYMPTVDFSASTAVSNNRYS |
| | | | | | GSNNLSQDNDAGENKVGLSFN |
| | | | | | LPLYSGGQTNSQVQQAQYNFV |
| | | | | | GASEQLESAHRSVVQTVRSSFN |
| | | | | | NVNASISSINAYKQAVVSAQSS |
| | | | | | LDAMEAGYQVGTRTIVDVLDA |
| | | | | | TTTLYNAKQQLSSARYTYLINQ |
| | | | | | LNIKSALGTLNENDLLLLNGAL |
| | | | | | GKPVSTSQDVVAPPTSAQDAYA |
| | | | | | DGYNDNAPASQTAAPAPVATR |
| | | | | | ASAPAITTTQPARSDSGNPFRN |
| 1 | WP_046372603.1 | outer | Serratia | 511 | MKKLLPLLIGLSLGGFSAMSQA |
| | | membrane | liquefaciens | | ENLLQVYKQARESNPDLRKTA |
| | | channel | | | ADRDAAFEKINEARSPLLPQLG |
| | | protein TolC | | | LSAGYTHINGYRDSRDQNSDA |
| | | | | | TSGSLALTQTIFDMSKWRALTL |
| | | | | | QEKTAGISDVTFQTSSQQLILDT |
| | | | | | ATAYFNVLSAIDSLSYTQAQKQ |
| | | | | | AVYRTLDQTTQRFNVGLVAITD |
| | | | | | VQNARSNYDTVLAAEVTARNT |
| | | | | | LDNALEKLRQVTGTFYPELASL |
| | | | | | NTDRFSTQRPEAVNNLLKEAES |
| | | | | | RNLSLLSARLSQDLAREQIRSAQ |
| | | | | | TGYMPTIDFNASTAVSNTKNNG |
| | | | | | SRNSTPDADIGQNKVGLSFNMP |
| | | | | | LYSGGQTNSQVQQAQYNFVGA |
| | | | | | SEQLESAHRNVVQTVRSSFNNV |
| | | | | | NASISSINAYKQAVVSAQSSLD |
| | | | | | AMEAGYQVGTRTIVDVLDATT |
| | | | | | TLYNAKQQLSSARYTYLINQLN |
| | | | | | IKFALGTLNENDLMLLNGALGK |
| | | | | | PISTSQDVVAPPTTAQDAYADG |
| | | | | | YQDNAPAQQSAAPAPVAATRT |
| | | | | | SAPVSTTTPPVRNSGNPFRN |
| 1 | WP_004948292.1 | outer | Serratia | 512 | MKKLLPLLIGLSLSGFSAMSQA |
| | | membrane | plymuthica | | ENLLQVYKQARESNPDLRKTA |
| | | channel | | | ADRDAAFEKINEARSPLLPQLG |
| | | protein TolC | | | LSAGYNRSYGYRDNRDQNGNT |
| | | | | | TSGSLALTQTIFDMSKWRALTL |
| | | | | | QEKTAGISDVTFQTSSQQLILDT |
| | | | | | ATAYFNVLSAIDSLSYTQAQKQ |
| | | | | | AVYRTLDQTTQRFNVGLVAITD |
| | | | | | VQNARSNYDTVLASEVTARNT |
| | | | | | LDNALEKLRQVTGTFYPELASL |
| | | | | | NTDRFSTQRPEAVNNLLKEAEA |
| | | | | | RNLSLLSARLSQDLAREQIKSA |
| | | | | | QTGYMPTVDFSASTAVSNNRYS |
| | | | | | GSRSISTDNDAGENKVGLSFNL |
| | | | | | PLYSGGQTNSQVQQAQYNFVG |
| | | | | | ASEQLESAHRSVVQTVRSSFNN |
| | | | | | VNASISSINAYKQAVISAQSSLD |
| | | | | | AMEAGYQVGTRTIVDVLDATT |
| | | | | | TLYNAKQQLSSARYTYLINQLN |
| | | | | | IKSALGTLNENDLLLLNGALGK |
| | | | | | PISTSQDVVAPPTTAQDAYAEG |
| | | | | | YNGNAPAPQTAAPAPVATRAS |
| | | | | | APAITTTQPARSNSGNPFRN |
| 1 | WP_040263333.1 | outer | Serratia | 513 | MKKLLPFLIGLSLGGFSAMSQA |
| | | membrane | symbiotica | | ENLLQVYKQARESNPELRKTAA |
| | | channel | | | DRDAAFEKINEARSPLLPQLGLT |
| | | protein TolC | | | AGYAYGRGYRDANNINSTMTN |
| | | | | | GSLALTQTIFDMSKWRVLTLQE |
| | | | | | KTAGISDVTFQTSSQRLILDTAT |
| | | | | | AYFSVLSAIDALSYTQAQKQAV |
| | | | | | YRTLDQTTQRFNVGLVAITDVQ |
| | | | | | NARSNYDTVLASEVTARNTLD |
| | | | | | NALEKLRQVTGTFYPELASLDT |
| | | | | | DRFNTQRPEAVNNLLKEAEAH |
| | | | | | NLSLLSARLSQDLAREQIRLAQT |
| | | | | | GYMPTINMSASTGITDTKYSGS |
| | | | | | SSSGDTDSNTGQNKIGLSINLPL |
| | | | | | YSGGATNSQVQQAQYSFVGAS |
| | | | | | ESLESAHRSLVQTVRSSFNNVN |
| | | | | | ASISSINAYKQAVVSAQSSMDA |
| | | | | | MEAGYQVGTRTIVDVLNATTT |
| | | | | | LYNAKQQLSSARYTYLINQLNI |
| | | | | | KSSLGTLNENDLLALNGALGKP |
| | | | | | VSTSPELLAPATTEQNAYANGY |
| | | | | | NGDGLATDTTQPARNSGDPFRH |
| 1 | WP_006086047.1 | MULTISPECIES: | Shewanella | 514 | MKFKIHNLCAALTLAASASAVQ |
| | | outer | | | ADDLLQIYQQALTSDPLVLQAQ |
| | | membrane | | | AQRNGLFEQIEQNRAPLLPTISA |
| | | channel | | | NVGYDKAWNDPKDDSSGLVGS |
| | | protein TolC | | | LKLNQVIYDHSAWVGLSLAEK |
| | | | | | AASQADSAYASALQNLITRITT |
| | | | | | AYFNVLTAKDNYEFQGAEKRAI |
| | | | | | ERQLEQTKQRFAVGLTAITDVH |
| | | | | | EAQAQYDLASATEILAENTLAN |
| | | | | | SYEALREITGIDHKTINVLDTNR |
| | | | | | FSAVTPAPTSSNEWLKIAETNSV |
| | | | | | DLMTQRIGKDIAQETIKLYQAG |
| | | | | | HMPSLSLNAGYNKGLEQKTGNI |
| | | | | | NEPDFDNVSVGVNLSIPIFEGFK |
| | | | | | VSSQVNQAQFQYVEASEKLEQ |
| | | | | | VYRGVVKNVRNNYNNVGASIS |
| | | | | | SIKAYEQSVISSESALKATQAGF |
| | | | | | EVGTRTIVDVLNRTRDLYDSKR |
| | | | | | KLSDARYSYINSVIALKQAAGT |
| | | | | | LNEDDVIAINNGLIAE |
| 1 | WP_011790662.1 | MULTISPECIES: | Shewanella | 515 | MKFKIRSLCAALTLAASAQAVQ |
| | | outer | | | ADDLLQIYQQALTNDPLVLQAQ |
| | | membrane | | | AQRDALFEKIEQNRAPLLPTLSA |
| | | channel | | | NVGYDKAWNDPRDNTSGLTGG |
| | | protein TolC | | | LKLNQVIYDHSAWVGLSLAEK |
| | | | | | AASQADSNYASTLQTLITRVTK |
| | | | | | AYFDVLTAKDNYEFQGAEKRAI |
| | | | | | ERQLEQTKQRFAVGLTAITDVH |
| | | | | | EAQAQYDLASASEILAENDLAN |
| | | | | | SYEALREITGIDHKTLNVLDTNR |
| | | | | | FSAVTPAPSSPSEWLKIAESNSV |
| | | | | | DLMTQRIGKDIAQETISLYKAG |
| | | | | | HMPSLSLSAGYNKGLEQKTGSV |
| | | | | | NDPDFDNVNVGLNLSIPIFEGFK |
| | | | | | VTSQVNEAQYQYVEASEKLEQ |
| | | | | | TYRSVVKNVRNNYNNVGASISS |
| | | | | | IRAYEQSVISSESALKATQAGFE |
| | | | | | VGTRTIVDVLNRTRDLYDSKRK |
| | | | | | LSDARYSYINSIVALKKAAGTL |
| | | | | | NEDDVIAINSGLKAE |
| 1 | WP_037422145.1 | MULTISPECIES: | Shewanella | 516 | MKFKIRSLCVALTLAASAPAVQ |
| | | outer | | | ADDLLQIYQQALTSDPLVLQAQ |
| | | membrane | | | AQRNALFEKIEQNRAPLLPTISA |
| | | channel | | | NVGYDKAWNDPRDDSSGLTGN |
| | | protein TolC | | | LKLNQVIYDHSAWVGLSLAEK |
| | | | | | AASQADSTYASALQNLITRVTK |
| | | | | | AYFDVLTAKDNYEFQGAEKRAI |
| | | | | | ERQLEQTKQRFAVGLTAITDVH |
| | | | | | EAQAQYDLASASEILAENTLAN |
| | | | | | SYEALREITGIDHKALNVLDTN |
| | | | | | RFSAVTPAPSSSSDWLKIAETNS |
| | | | | | VDLMTQRIGKDIAQETISLYKA |
| | | | | | GHMPSLSLNAGYSKGLEQKLG |
| | | | | | NSNEPDFDNMSAGINLSIPIFEGF |
| | | | | | KVSSQVKEAQFQYVEASEKLEQ |
| | | | | | TYRSVVKNVRNNYNNVGASISS |
| | | | | | IRAYEQSVISSESALKATQAGFE |
| | | | | | VGTRTIVDVLNRTRDLYDSKRK |
| | | | | | LSDARYSYINSIVALKQAAGTL |
| | | | | | NEDDVIAINNGLKAE |
| 1 | WP_025009358.1 | outer | Shewanella algae | 517 | MKFKIRTLCAALTLAVSAPAVQ |
| | | membrane | | | ADDLLQIYQQALTNDPVVLQA |
| | | channel | | | KAQRDQLYEKIEENRAPLLPTIS |
| | | protein TolC | | | ANVGYDKAWNDPSADTSGLTG |
| | | | | | AIKLNQVIYDHSAWVGLSLAEK |
| | | | | | AASQADAVYASALQTLITRVTT |
| | | | | | AYFDVLAAKDNYEFKGAEKRA |
| | | | | | IERQLEQTKQRFAVGLTAITDV |
| | | | | | HEAQAQYDLATADEILAENQLI |
| | | | | | NSYEALREITGIDHKSIDILDTNR |
| | | | | | FSAASPTPAVPGDWLKMAETNS |
| | | | | | VDLLTQRIGKDIAQETISLYKAG |
| | | | | | HMPSLSLSAGYTKGLEQEIGSQ |
| | | | | | SQPDFDNGTVGVNLSVPIFEGF |
| | | | | | KVSSQVKQAQYAYVEASQKLE |
| | | | | | QTYRQVVKNVRNNFNNVGASI |
| | | | | | SSIKAYEQSVLSSESALKATQAG |
| | | | | | FEVGTRTIVDVLNRTRDLYDSK |
| | | | | | RQLSNARYGYIKSILALKQATG |
| | | | | | TLNEDDVISINNGLTAPAQQ |
| 1 | WP_112351746.1 | outer | Shewanella | 518 | MKFKIRTICAALTLAFSTSALQA |
| | | membrane | benthica | | DDLLQIYQQALTSDPIALQAQA |
| | | channel | | | RRDALYEQIEENRAPLLPTISAS |
| | | protein TolC | | | VGYNKSWHNEDTMSNTDSSGI |
| | | | | | NAGVTLNQVIYDHSAWVGLSL |
| | | | | | AELAASQADAAYASSLQTLIIR |
| | | | | | VTSAYFAVLSAKDTFEFQGSEK |
| | | | | | RAIERQLKQTKQRFAVGLTAIT |
| | | | | | DVHEAQAQYDLARAQEILAEN |
| | | | | | ELTNSYEALREITGIEHRTINILD |
| | | | | | TDRFSAVSPSPTRAIDWLKIAET |
| | | | | | NSVDLLTTRIGKDIAKETISLYK |
| | | | | | AGHMPTLSLNAGYTTNIQQENN |
| | | | | | DNDANDFDNGTVGLTLSIPIFEG |
| | | | | | FKISSKVNQAQYQYVEASEKME |
| | | | | | QVHRKVVKDIRNNFNNVGASIS |
| | | | | | SIRAYEQSVISSESALKATQAGF |
| | | | | | EVGTRTIVDVLNRTRDLYDSKR |
| | | | | | KLSDARYGYINSILALKQAAGT |
| | | | | | LNEDDVITINQGLIAQAQATQN |
| | | | | | TTQ |
| 1 | WP_011073650.1 | outer | Shewanella | 519 | MKFKIRSLCVALTLAASAHAVQ |
| | | membrane | oneidensis | | ADDLLQIYQQALTNDPLVLQAQ |
| | | channel | | | AQRNALFEKIEQNRAPLLPTISA |
| | | protein TolC | | | NVGYDKAWNDPRDDSSGLTGS |
| | | | | | LKLNQVIYDHSAWVGLSLAEK |
| | | | | | AASQADSTYASALQNLISRVTK |
| | | | | | AYFDVLTAKDNYEFQGAEKRAI |
| | | | | | ERQLEQTKQRFAVGLTAITDVH |
| | | | | | EAQAQYDLASASEILAENTLAN |
| | | | | | SYEALREITGIDHKALNVLDTN |
| | | | | | RFSAVTPAPSSSTDWLKIAETNS |
| | | | | | VDLMTQRIGKDIAQETISLYKA |
| | | | | | GHMPSLSLNAGYAKGLEQKLG |
| | | | | | DASEPDFDNMSVGINLSVPIFEG |
| | | | | | FKVSSQVKEAQFQYVQASERLE |
| | | | | | QTYRGVVKNVRNNYNNVGASI |
| | | | | | SSIRAYEQSVISSESALKATQAG |
| | | | | | FEVGTRTIVDVLNRTRDLYDSK |
| | | | | | RKLSDARYSYINSIVALKQAAG |
| | | | | | TLNEDDVIAINNGLKAE |
| 1 | WP_142873921.1 | outer | Shewanella sp. | 520 | MKFKIRTICAALTLAVTTSAVQ |
| | | membrane | YLB-08 | | ADDLLQIYQQALTSDPIALQAQ |
| | | channel | | | AQRDTLYQQIEENRAPLLPTISA |
| | | protein TolC | | | SVGYGKSWHNESSADTDSTGL |
| | | | | | NAGVTLNQVIYDHSAWVGLSL |
| | | | | | AELAASQADAAYASSLQNLIIR |
| | | | | | VTSAYFEVLSAKDTFEFQGSEK |
| | | | | | RAIERQLEQTKQRFAVGLTAIT |
| | | | | | DVHEAQAQYDLARASEILAEN |
| | | | | | QLINSYEALREITGIDHKSINILD |
| | | | | | TNRFSAVTPSPAKSNDWLKMA |
| | | | | | ESNSVDLLTTRIGKDIAQETISL |
| | | | | | YKAGHMPSLNLNAGYKTNIQQ |
| | | | | | ENQNGSQPDFDNANIGITLSVPI |
| | | | | | FEGFKVSSKVNQAQYQYVEAS |
| | | | | | EKMEQAYRKVVKDVRNNENN |
| | | | | | VGASISSIRAYEQSVISSESALKA |
| | | | | | TQAGFEVGTRTIVDVLNRTRDL |
| | | | | | YDSKRQLSDARYGYINSIMALK |
| | | | | | QAAGTLNEDDVIAINNGLIAEA |
| | | | | | VTP |
| 1 | NP_708846.2 | outer | Shigella flexneri | 521 | MKKLLPILIGLSLSGFSSLSQAE |
| | | membrane | 2a str. 301 | | NLMQVYQQARLSNPELRKSAA |
| | | channel | | | DRDAAFEKINEARSPLLPQLGL |
| | | protein | | | GADYTYSNGYRDANGINSNAT |
| | | | | | SASLQLTQSIFDMSKWRALTLQ |
| | | | | | EKAAGIQDVTYQTDQQTLILNT |
| | | | | | ATAYFNVLNAIDVLSYTQAQKE |
| | | | | | AIYRQLDQTTQRFNVGLVAITD |
| | | | | | VQNARAQYDTVLANEVTARNN |
| | | | | | LDNAVEQLRQITGNYYPELAAL |
| | | | | | NVENFKTDKPQPVNALLKEAE |
| | | | | | KRNLSLLQARLSQDLEREQIRQ |
| | | | | | AQDGHLPTLDLTASTGISDTSYS |
| | | | | | GSKTRGAAGTQYDDSNMGQN |
| | | | | | KVGLSFSLPIYQGGMVNSQVKQ |
| | | | | | AQYNFVGASEQLESAHRSVVQ |
| | | | | | TVRSSFNNINASISSINAYKQAV |
| | | | | | VSAQSSLDAMEAGYSVGTRTIV |
| | | | | | DVLDATTTLYNAKQELANARY |
| | | | | | NYLINQLNIKSALGTLNEQDLL |
| | | | | | ALNNALSKPVSTNPENVAPQTP |
| | | | | | EQNAIADGYAPDSPAPVVQQTS |
| | | | | | ARTTTSNGHNPFRN |
| 1 | WP_015047577.1 | TolC family | Simiduia | 522 | MFSRTPLAASLRWICAAGLLSA |
| | | outer | agarivorans | | APLSMADTLNQIYELAVKNDH |
| | | membrane | | | QIKADEAAYRSAREAKTIARAN |
| | | protein | | | LLPLISANASYSQSDQDITSSGV |
| | | | | | GPVPTKTNVQSWGATLAQPLL |
| | | | | | NMPAWFGYKQGEELTNKAEAD |
| | | | | | FSAAQQDLIVRTATAYFNVLRA |
| | | | | | IDTLEASIAEENANSHQLAQTK |
| | | | | | QRFEVGLTAITDVHEAQASYDI |
| | | | | | ATAQTLTDRGNLGIAFEALEVL |
| | | | | | TGKPHDKVAPLKEEFPVLKPQP |
| | | | | | EARSEWVEFALQNNYSLKSAKF |
| | | | | | ASEASRRGAQASQAAHYPTLTA |
| | | | | | SLSYNDSDRDIQDTILGDNVTT |
| | | | | | GSSAAISLNVPIYEGGGTSARSR |
| | | | | | QAAENAMQSQELFNKTQRDTI |
| | | | | | QAARSLHLQVTTDVARVAARK |
| | | | | | QAITSSLSAVQATQAGYEVGTR |
| | | | | | NLVDVLQVQRTLYQARRDYSN |
| | | | | | SLYDYVINLFRLKAVAGNLTPA |
| | | | | | DVVQINQWLDEAAAQNRAKYE |
| 1 | WP_016352548.1 | TolC family | Spiribacter salinus | 523 | MRHNIRLHRPLALLITIGLASAP |
| | | outer | | | LGVSAEDTLIEIYDRARQSDPQF |
| | | membrane | | | QQAIADRRAREEALPQARAGLR |
| | | protein | | | PDVSLSSRYTTRDTDSDIGTTTG |
| | | | | | SEEEDYRQLSYGVELTQPLYRF |
| | | | | | SQARSVDRADALVEQARADFA |
| | | | | | SAEQALIIRTAERYFSVLDARES |
| | | | | | VDAAAANLEAIERQLEQAEQRF |
| | | | | | EVGVIARTDVEEARAQADLAR |
| | | | | | AELLQAEDDFETERERLRELTD |
| | | | | | RAPARLRQVREGVELNAPSPND |
| | | | | | PDAWRGRAEDENRDLAAARFA |
| | | | | | AEAAMEGVDVERGGRFPQIDLI |
| | | | | | AGYDGLEQYDRDGQDRSSEEY |
| | | | | | SAGIQLNLPLYQGGGVSSSIREA |
| | | | | | QFRYTEAREALEEARRTVTRNA |
| | | | | | ADAYRGVETALERVRALEQARI |
| | | | | | STQAALDATEAGFEVGTRTIVD |
| | | | | | VLNAQREVFNAERDYQQARHA |
| | | | | | YLVNTLRLQQAAGALSAEDLR |
| | | | | | GVDTLLGNGNG |
| 1 | WP_005410961.1 | MULTISPECIES: | Stenotrophomonas | 524 | MIRRSLAVALATALLPLSAHAA |
| | | TolC | | | DLLQVYEMARNGDPQLSAAES |
| | | family outer | | | TRLVDKEGAVQARAALLPQING |
| | | membrane | | | QATLNRTRSEPNADINSGSFTN |
| | | protein | | | KRRNYTIDGSQTLFNWTQINNL |
| | | | | | RSQRELSKAADFTLESANDSLIV |
| | | | | | RTSAAYFNVLVAIESLNAAQTN |
| | | | | | EAAAKKQFDFADKRLEVGLAPI |
| | | | | | TDVHEARAQYDQARANTIVAQ |
| | | | | | NTLADNYQALTELTGQPVVNL |
| | | | | | RGLPADFRPEVPANRGNIDELV |
| | | | | | HQATTQNPALKAQELKVSAAE |
| | | | | | AGVQAARGGHYPTLSLGGSWG |
| | | | | | KTATWGDSTGSGSLSPDARTNS |
| | | | | | IGLTLNVPIFAGGATQSGVRQA |
| | | | | | LAQRDIAQDGYEQQKRALDRN |
| | | | | | TRNAYQTLVQGISEVEARRLAV |
| | | | | | VSAQSAYDASQVGLEVGTRTV |
| | | | | | LDVIQNQRILFSAQLDYAQARY |
| | | | | | TFLQNRLLLSQSLGALDVAELQ |
| | | | | | DVNRLLTQDAGNPATTTH |
| 1 | WP_014648347.1 | MULTISPECIES: | Stenotrophomonas | 525 | MIRRSLAVALATALLPLSAHAA |
| | | TolC | | | DLLQVYEMARNGDPQLSAAES |
| | | family outer | | | TRLYDKEGAVQARAALLPQING |
| | | membrane | | | QAQLNRTRSEPNADANSGSFTN |
| | | protein | | | KRRTYTIDGSQTLFNWTQINNL |
| | | | | | RSQRELSKAADFTLDSANDSLI |
| | | | | | VRTSAAYFNVLVAIESLNAAQT |
| | | | | | NEAAAKKQFDFADKRLEVGLA |
| | | | | | PITDVHEARAQYDQARANTIVA |
| | | | | | QNTLADNYQALTELTGQPVTN |
| | | | | | LRGLPADFRPEVPANRGNIDEL |
| | | | | | VQQATTQNPALKAQELKVSAA |
| | | | | | EAGVQAARGGHYPTLSLGGSW |
| | | | | | GKSATWGDSTGSGSLSPDARTN |
| | | | | | SIGLTLSVPIFSGGATQSGVRQA |
| | | | | | LAQRDIAQDGYEQQKRALDRN |
| | | | | | TRNAYQTLVQGISEVEARRLAV |
| | | | | | VSAQSAYDASQVGLEVGTRTV |
| | | | | | LDVIQNQRILFSAQLDYAQARY |
| | | | | | TFLQNRLLLSQSLGALDVAELQ |
| | | | | | DVNRLLTQDAGNPATTTH |
| | WP_015260353.1 | TolC family | Thioalkalivibrio | 526 | MKMLRALCATAVFAATWALP |
| | | outer | nitratireducens | | AQAEDLLQVFEHAQMEDAQLR |
| | | membrane | | | AAEAQYRAVLEARPIARSTLLP |
| | | protein | | | QLSADAELGAFYSDPDGSSSID |
| | | | | | GSSHSIGLNLNQSLFDQRNRIGV |
| | | | | | RQADLQISSAAAELDAARQDLI |
| | | | | | LRVAEAYFGVLVARETLEFRRA |
| | | | | | EREAISRQLEQTQRRFEVGLIAI |
| | | | | | TDVKEAQAQFDIASAEQIAAEN |
| | | | | | ALNLAREQLAVITNRYYDQLA |
| | | | | | ALGEHLDMPSPDPMDPQDWVS |
| | | | | | AALENNQELNAQRLSAEVARE |
| | | | | | QIGRQRAEGLPTLGLGASVSDT |
| | | | | | GYSGVNSVPGGQFNDRTDAQIG |
| | | | | | LRLDVPLYTGGRVSAITREARE |
| | | | | | QFEAAQETVVFTQRQTVQNTR |
| | | | | | NSYLSVIANASRARALAQALQS |
| | | | | | TQAAFESAQAGFEVGTRTQVD |
| | | | | | VLLALREVFRAERDYAEARYG |
| | | | | | YLLETLRLQRAVGSLSLADLQR |
| | | | | | INAFLE |
| 1 | WP_068576483.1 | MULTISPECIES: | unclassified | 527 | MLRKLSLAIAVSCASNGMAWA |
| | | TolC | Pseudomonas | | AEAPLTTTKTDLVSVYQEAVDN |
| | | family outer | | | NADLAAARAQYGAQKEVVPQ |
| | | membrane | | | ARAGLLPNLSAGADLNNTRTKL |
| | | protein | | | DEPAMTANRSGNVYRATLAQP |
| | | | | | LFRADRWFQLQAAKEVNEQAA |
| | | | | | LQLSATEQNLILQSAENYFAVL |
| | | | | | RAQDNLASTKAEEAAFKRQLD |
| | | | | | QSNERFDVGLSDKTDVLQSQAS |
| | | | | | YDTARANRIVAQRQVDDAFEA |
| | | | | | LITLTNREYNSIQGIVHTLPVLA |
| | | | | | PVPNDAKAWVDTAAKQNLNLL |
| | | | | | ASNYAVSAAEETLKQRKAGHA |
| | | | | | PTLDAVVQYKKGDNDALGFSN |
| | | | | | PNPNPIAPPYHGDVEQRSIGLQL |
| | | | | | NIPIYSGGLTSSQVRESYSRLSQS |
| | | | | | EQQRESLRRQVVENTRNLHRA |
| | | | | | VNTDVEQVQARRQSIISNQSAV |
| | | | | | EATEIGYQVGTRNIVDVLDAQR |
| | | | | | QLYNSVRNYNNSRYDYILDNLR |
| | | | | | LKQAAGTLNPGDLQDLARYLK |
| | | | | | ADYNPDRDFLPPDLAKAAAEQ |
| | | | | | LKARP |
| 1 | WP_068583080.1 | MULTISPECIES: | unclassified | 528 | MLRRHSVIVAVSYIFSSAACAA |
| | | TolC | Pseudomonas | | NTPTTVNNDLVSVYEEAVDNN |
| | | family outer | | | ADLAVARADYSARREVVPQAR |
| | | membrane | | | AGLLPNLSAGAEMMNTRTKLD |
| | | protein | | | EPSVTSNRSGNSWSATLAQPIFR |
| | | | | | ADRWFQLQAAEAVNEQAALQL |
| | | | | | SATEQSLIRQTAETYFAVLRAQ |
| | | | | | DNLASTKAEESAFKHQLEQSNE |
| | | | | | RFDVGLSDKTDVLQSQASYDT |
| | | | | | ARANRIVAERQVQDAFEALVTL |
| | | | | | TNRQYTAIQGVVHTLPVQVPTP |
| | | | | | NDAKAWVEAAGRQNLNLLAS |
| | | | | | NHAVTAAEETVRQRKAGHAPT |
| | | | | | LDAVVKYQKGDNDSLGFTNPS |
| | | | | | QTGVRYGGDVEQTSVGLQLNIP |
| | | | | | IYSGGLTSSQVREAYSRLTQSEQ |
| | | | | | QRESLRRQVVESTRNLHRAVNT |
| | | | | | DVEQIQARKQSIVSNQSALEAT |
| | | | | | EVGYQVGTRNIVDVLDAQRQL |
| | | | | | YASVRDYNNSRYDYILDNLRLK |
| | | | | | QAAGTLSPQDLRDLGRYLKVN |
| | | | | | YNPDKDFLPPDLAVEAKKNFGS |
| | | | | | RP |
| 1 | WP_141698231.1 | MULTISPECIES: | unclassified | 529 | MHLSPRALAGCLLILLSPFAGLV |
| | | TolC | Pseudomonas | | RATEPQAMPSDLWRVYQDARH |
| | | family outer | | | NNSELAAARADQAARAEAVPQ |
| | | membrane | | | ARAGLLPTLSASAEHNGTSTSL |
| | | protein | | | QQPRQATRRSGTSYQAVLNQP |
| | | | | | VFRAERWFGLKAAEAEDQQAM |
| | | | | | LELAAAEQKLMLDSAQAYFGL |
| | | | | | LKAQDALAAATAEEAALKRQL |
| | | | | | ALAQRGLQLGLSDRTDVLQAE |
| | | | | | AGHDTAQANRIVARKRTDDAF |
| | | | | | EALDTLTHQQYPALQGLRHDM |
| | | | | | PVLLPEPNDARRWVDSAVRQN |
| | | | | | LTLQASQYALEATQQTLKARK |
| | | | | | AGHAPTLDAVLRYQVGDNDNL |
| | | | | | GYGNSDIRGSGYGGDVQQRSV |
| | | | | | GLQLNIPLFSGGQTSSQVREAY |
| | | | | | QRMSQRESLNDNLRRQVVEQT |
| | | | | | RNLHRGLNSGADQVRARRQSII |
| | | | | | SNQGAVLASQLGLQVGTRNIVD |
| | | | | | VLEAQRQLYNAVRQYNDSRYD |
| | | | | | YILDTLRLKQAAGTLSPEDLKA |
| | | | | | LCDYLKADYDPDRDFLPPEFPR |
| | | | | | RLAAR |
| 1 | WP_179571380.1 | MULTISPECIES: | unclassified | 530 | MLRRLSLAIAVAAASVGLVQA |
| | | TolC | Pseudomonas | | AEAPLSSKTDLVTVYQEAAKN |
| | | family outer | | | NADIAAARADYQARREVVPQA |
| | | membrane | | | RAGLLPNLSAGANYGDTRTEID |
| | | protein | | | SPSATISRSGLVYQANLSQPLFR |
| | | | | | ADRWFQLQAAEATSEQAALEL |
| | | | | | SATEQNLILQSAETYFAVLRAQ |
| | | | | | DNLASTRAEESAFKRQLDQANE |
| | | | | | RFDVGLSDKTDVLEAQAGFDT |
| | | | | | ARANRLLAERAVDDAFEALVT |
| | | | | | LTNRDYVAVEGIVHTLPVLAPT |
| | | | | | PNDAKSWVDTAAAQNLNLQAS |
| | | | | | LYAVNAAEENLRQRKAGHAPT |
| | | | | | LDAVASYQKGDNDSLGFTNSG |
| | | | | | SPLSPSYSGDVSQRTIGLQLNIPL |
| | | | | | YSGGLTSSQVREAYQRLGQTEQ |
| | | | | | LRESLRRQVVQNTRNLFRAVNT |
| | | | | | DVETVQARRQSIISNQSALEATE |
| | | | | | IGYQVGTRNIVDVLDAQRQLYS |
| | | | | | AVRNYNDARYDYILNNLRLKQ |
| | | | | | AAGTLSPADLEALGSFLKPDYN |
| | | | | | PDKDFLPPDLASAAEERLKNNQ |
| | | | | | DF |
| 1 | WP_132923472.1 | MULTISPECIES: | unclassified | 531 | MKKLLPLLIGLSLGGFSTLSQAE |
| | | outer | Sodalis | | NLLQVYQQARLSNPDLRASAA |
| | | membrane | | | DRDAAFEKINEARSPLLPQLGL |
| | | channel | | | GVDYTYNNGFRDQRDINSNETS |
| | | protein TolC | | | GSLQLTQTIFDMSKWRALTVQE |
| | | | | | KTAGVQDVTYQTQQQTLILNT |
| | | | | | ATAYFNVLSAIDALSYTEAQKQ |
| | | | | | SIYRELDQTTQRFNVGLVAITD |
| | | | | | VQNARSNYDTVLADEVNARNT |
| | | | | | VDNALESLRQISGNFYPQLASL |
| | | | | | NIDRFKAAQPQPINALLKEAEM |
| | | | | | RNLTLLSARLSQDVAREQIRLEE |
| | | | | | TGHYPTLALTASSSLSNTDYNG |
| | | | | | SRTNGNANYNNTNIGQNAIGLS |
| | | | | | LSVPIYSGGAVTSQVKQAQYSF |
| | | | | | VSASEQLESAHRSVVQTVRSSF |
| | | | | | NNISASISSIDAYRQAVISAQSSL |
| | | | | | DASEAGYEVGTRTIVDVLDATT |
| | | | | | TLYDAKRNLSTARYNYVINQLT |
| | | | | | LKNALGTLNEDDIRTLNASLGQ |
| | | | | | PIATSPESVAPVNPNQDANAEG |
| | | | | | VNPGQTSRAPRSNPSDAAQSAR |
| | | | | | QSATPVSATVPASSSAKKRNPF |
| | | | | | QH |
| 1 | WP_005379884.1 | MULTISPECIES: | Vibrio | 532 | MKKLLPLFISAALGSMSTSAFA |
| | | outer | | | DSLAEIYDLAKQNDPQLLSVAA |
| | | membrane | | | QRDQAFEAITSSRSALLPQINLT |
| | | channel | | | AGYNLTRGDTEYDSNLISDVSN |
| | | protein TolC | | | DGNALTAGVSFSQALYNRASWI |
| | | | | | SLDTAEKSARQADATYAATQQ |
| | | | | | ALILRVSQAYFEVLRAQDNLVF |
| | | | | | VRAEKAAVGRQLEQTKQRFEV |
| | | | | | GLSAITDVHDAQAQYDQVLAD |
| | | | | | EVLAENALTNSYESLREITGQE |
| | | | | | HKDLNILDTGRFTASPSTVPAES |
| | | | | | LIDEAKTKNLTLLSSRILQDIAR |
| | | | | | DNISLASSGHLPTLSLDGGYNY |
| | | | | | GDTSHSSRDNTTDNFNIGVNLA |
| | | | | | VPLYTGGNVTSQTKQAEYAYV |
| | | | | | AASEDLEAQYRSVVKDVRAQN |
| | | | | | NNINASIGALRAYEQAVISARSA |
| | | | | | LEATEAGFDVGTRTIVDVLDAT |
| | | | | | RRLYDANKNLSNARYDYILSVL |
| | | | | | QLRQAVGTLSEQDILDVDAGLK |
| | | | | | AAK |
| 1 | WP_005450392.1 | MULTISPECIES: | Vibrio | 533 | MKKLLPLFISAALGSMSTSAWA |
| | | outer | | | DSLAEIYDLAKQNDPQLLSVAA |
| | | membrane | | | QRDQAFEAINSSRSALLPQINLT |
| | | channel | | | AGYDITRGDRDLDAGGKTSTDE |
| | | protein TolC | | | NVLSAGVGFTQELYNRASWITL |
| | | | | | DTAEKTARRADANYAATQQGL |
| | | | | | ILRVSQAYFEVLRAQDNLVFVR |
| | | | | | AEKAAVGRQLEQTKQRFEVGL |
| | | | | | SAITDVHDAQAQYDAVLADEV |
| | | | | | LAENSLINSYESLREITGQEHKN |
| | | | | | LNVLDTARFSASRSSEAAETLIE |
| | | | | | EAKTTNLSLLSARIGQDIARDNI |
| | | | | | SLASSGHLPTLSLDGGYNYADT |
| | | | | | SESVTDNTTDGFKVGVNLAVPL |
| | | | | | YTGGRTTSEVKQAEYGYVSASE |
| | | | | | ELEKEYRSVVKEVRAQNNNINA |
| | | | | | SIGALKAYEQSVVSAQSALEAT |
| | | | | | EAGFDVGTRTIVDVLDATRRLY |
| | | | | | DANKNLSDARYNYILAGLQLR |
| | | | | | QAVGTLSEQDILDVDAGLKPAK |
| 1 | WP_005455451.1 | MULTISPECIES: | Vibrio | 534 | MKKLLPLFISAALGGISSSAWA |
| | | efflux | | | DSLAEIYDLAKQNDPQLLSVAA |
| | | RND | | | QRDRAFEAITSSRSALLPQINLT |
| | | transporter | | | AGYNLTRGDTEYDSNLISDVSN |
| | | outer | | | DSNALTAGVNFSQELYNRASWI |
| | | membrane | | | TLDTAEKSARQADATYAAAQQ |
| | | protein VpoC | | | GLILRVSQAYFEVLRAQDNLVF |
| | | | | | VRAEKAAVGRQLEQTKQRFEV |
| | | | | | GLSAITDVHDAQAQYDAVLAD |
| | | | | | EVLAENDLINSYESLREITGQEH |
| | | | | | KNLNVLDTNRFSATRTNSPAET |
| | | | | | LIDEAKTKNLSLLSARISQDIAR |
| | | | | | DNISLASSGHLPTLSLDGGYNY |
| | | | | | GDTSNSARDNTTDNFNIGVNLA |
| | | | | | VPLYTGGNVTSQTKQAEFAYV |
| | | | | | AASEDLEAQYRSVVKDVRAQN |
| | | | | | NNINASIGALKAYEQSVVSARS |
| | | | | | ALEATEAGFDVGTRTIVDVLDA |
| | | | | | TRRLYDANKNLSDARYNYILSV |
| | | | | | LQLRQAVGTLSEQDILDVDAGL |
| | | | | | KPAK |
| 1 | WP_005497054.1 | MULTISPECIES: | Vibrio | 535 | MKKLLPLFISAALGGISSSAWA |
| | | outer | | | DSLAEIYDLAKQNDPQLLSVAA |
| | | membrane | | | QRDRAFEAITSSRSALLPQINLT |
| | | channel | | | AGYNLTRGDTDYDDSALGKSS |
| | | protein TolC | | | NDQDALTAGVSFSQTLYNRAS |
| | | | | | WISLDTAEKSARQADATYAAT |
| | | | | | QQGLILRVSQAYFEVLRAQDNL |
| | | | | | VFVRAEKAAVGRQLEQTKQRF |
| | | | | | EVGLSAITDVHDAQAQYDAVL |
| | | | | | ADEVLAENDLINSYESLREITGQ |
| | | | | | EHKNLNVLDTNRFSATRTSSPA |
| | | | | | ETLIEEAKTKNLSLLSARISQDIA |
| | | | | | RDNISLASSGHLPTLSLDGGYN |
| | | | | | YGDYSGTESNYNSGLGRNANS |
| | | | | | DRTSDGFNIGVNLAVPLYTGGN |
| | | | | | VTSQTKQAEFAYVAASEDLEA |
| | | | | | QYRSVVKDVRAQNNNINASIGA |
| | | | | | LKAYEQSVVSARSALEATEAGF |
| | | | | | DVGTRTIVDVLDATRRLYDAN |
| | | | | | KNLSDARYNYILSVLQLRQAVG |
| | | | | | TLSEQDILDVDAGLKPAK |
| 1 | WP_017046499.1 | MULTISPECIES: | Vibrio | 536 | MKKLLPLFISAALGSLSSSVWA |
| | | outer | | | DTLTEIYNQAKENDPQLLSSAA |
| | | membrane | | | QRDAAFEAVTSSRGDLLPQINL |
| | | channel | | | TAGYNINRSDIDARESDKLTAGI |
| | | protein TolC | | | SFSQQLYQRSSWVSLDTAEKNA |
| | | | | | RKADSAYAATQQGLILRVAQA |
| | | | | | YFEVLRAKDNLAFVRAEKAAV |
| | | | | | ARQLEQTKQRFEVGLSAITDVH |
| | | | | | DAQAQYDGVLADEVLAKNSLT |
| | | | | | NSYETLREITGQEHSDLSVLDTN |
| | | | | | RFSASKTTQPIDALLEEAQQKN |
| | | | | | LSLLTARIAQDVAKDNISLASSG |
| | | | | | HLPSLTLDGGYKYGDESNNNSG |
| | | | | | SKGDYNDFNVGINLAVPLYTGG |
| | | | | | KTTSQTKQAEFAYVAASQDLE |
| | | | | | KTYRSVVKDVRAFNNNINASIG |
| | | | | | ALRAYEQSVISAKSALEATEAG |
| | | | | | FDVGTRTIVDVLDSTRRLYDAN |
| | | | | | KNLSNARYDYVLSVLQLRQAV |
| | | | | | GTLSEQDILDINAGLKADS |
| 1 | WP_017820248.1 | outer | Vibrio | 537 | MKKLLPLFISAALGSMSTSAFA |
| | | membrane | alginolyticus | | DSLAEIYDLAKQNDPQLLSVAA |
| | | channel | | | QRDQAFEAITSSRSALLPQINLT |
| | | protein TolC | | | AGYNLTRGDNDYNDGALGKSS |
| | | | | | NDQDALTAGVSFSQTLYNRAS |
| | | | | | WISLDTAEKSARQADATYAAT |
| | | | | | QQALILRVSQAYFEVLRAQDNL |
| | | | | | VFVRAEKAAVGRQLEQTKQRF |
| | | | | | EVGLSAITDVHDAQAQYDQVL |
| | | | | | ADEVLAENALTNSYESLREITG |
| | | | | | QEHKDLNILDTGRFTASPSTVPA |
| | | | | | ESLIDEAKTKNLTLLSSRILQDIA |
| | | | | | RDNISLASSGHLPTLSLDGGYN |
| | | | | | YGDYSGTDNTFNSGLNRNANS |
| | | | | | DRTSDNFNIGVNLAVPLYTGGN |
| | | | | | VTSQTKQAEYAYVAASEDLEA |
| | | | | | QYRSVVKDVRAQNNNINASIGA |
| | | | | | LRAYEQAVISARSALEATEAGF |
| | | | | | DVGTRTIVDVLDATRRLYDAN |
| | | | | | KNLSNARYDYILSVLQLRQAVG |
| | | | | | TLSEQDILDVDAGLKAAK |
| 1 | WP_086982190.1 | outer | Vibrio aphrogenes | 538 | MKKLLPLFISLSLGGIHLAHADD |
| | | membrane | | | LAQIYNQAKENDPQLLSSKADR |
| | | channel | | | DAAFEAIESSRGDLLPQINLTAG |
| | | protein TolC | | | YNINRNINSHNNDPVESYGAST |
| | | | | | SEQNVFTAGLDFSQQLYSRDSW |
| | | | | | VNLDIAEQNARQQDSAYAAAQ |
| | | | | | QEMILRVSQAYFDVLQAQDNL |
| | | | | | TFIQAEKKAVGRQLEQTKQRFE |
| | | | | | VGLSAITDVHDAQAQYDSVLA |
| | | | | | DEIIAQNDLTNSYEALREITGQD |
| | | | | | NKDLRVLDTKRFTASTPQTTQA |
| | | | | | SLVDEAKQKNLSLLTARIAQDI |
| | | | | | AKSNISLAQSGYSPSLTLDGGY |
| | | | | | QYSDIDEHNSHSPYSNYTSNDY |
| | | | | | NIGVNLVIPLYQGGSTSADVKT |
| | | | | | AQYQYVSTSQQLEETYRSVVK |
| | | | | | DVRAYYNNINASIGTIRAYQQS |
| | | | | | VISAKSALEATEAGYEVGTRTIV |
| | | | | | DVLDSTRSLYDANRNLSDARY |
| | | | | | NYILSVLQLKQAVGTLSEQDVL |
| | | | | | DINAGLIAAK |
| 1 | WP_086960792.1 | outer | Vibrio casei | 539 | MKKLLPLLISLSLGGTHLAHAD |
| | | membrane | | | DLAEIYNQAKENDPLLLSSKAD |
| | | channel | | | RDAAFEAINSSRGVLLPQINLTA |
| | | protein TolC | | | GYNITRNIDSHDSHRPSAYTDE |
| | | | | | NNNFTAGLDFSQQLYQRDSWV |
| | | | | | NLDIAEQNARQQDSSYASQQQS |
| | | | | | LILRVSQAYFGVLQAQDNLSFV |
| | | | | | SAEKKAVGRQLEQTKQRFDVG |
| | | | | | LSAITDVHDAQAQYDSVLADEI |
| | | | | | LAQNDLTNSYEELREITGQDNK |
| | | | | | DLRILDTKRFSASTPQTTQNSLV |
| | | | | | DEAQTKNLDLLTARIAQDIAKS |
| | | | | | NISLANSGYTPSLTLDAGYQYS |
| | | | | | DVDAHNDHSSLNNYKSNDYNV |
| | | | | | GINLVVPLYQGGSTSADVKTAQ |
| | | | | | YQYVSASQQLEATYRGVVKNV |
| | | | | | RAYYNNINASIGTIRAYQQSVV |
| | | | | | SAKSALEATEAGYEVGTRTIVD |
| | | | | | VLDSTRSLYDANRNLSDARYN |
| | | | | | YILSVLQLKQAVGTLSEQDILDI |
| | | | | | NTGLVAAK |
| 1 | WP_000735329.1 | outer | Vibrio cholerae | 540 | MKKLLPLFVSAALGTLSSAVW |
| | | membrane | | | AENLAEIYNQAKENDPQLLSVA |
| | | channel | | | AQRDAAFEAVTSSRSALLPQIN |
| | | protein TolC | | | LTAGYNINRSDQAPRESDLLSA |
| | | | | | GINFSQELYQRSSWVSLDTAEK |
| | | | | | KARQADSQYAATQQGLILRVA |
| | | | | | KAYFEVLRAQDNLEFVRAEKA |
| | | | | | AVGRQLEQTKQRFEVGLSAITD |
| | | | | | VHDAQAQFDGVLADEVLAENS |
| | | | | | LTNSYEALREITGQEYSKLAVL |
| | | | | | DTKRFAASRTTESSEALIEKAQQ |
| | | | | | QNLSLLAARISQDVARDNISLAS |
| | | | | | SGHLPSLTLDGGYNYGNNSND |
| | | | | | NAKNTSGEEYNDFKIGVNLKVP |
| | | | | | LYTGGNTTSLTKQAEFAYVAAS |
| | | | | | QDLEAAYRSVVKDVRAYNNNI |
| | | | | | NASIGALRAYEQAVISAKSALE |
| | | | | | ATEAGFDVGTRTIVDVLDATRR |
| | | | | | LYDANKNLSNARYDYILSVLQL |
| | | | | | RQAIGTLSEQDVMDVNAGLKV |
| | | | | | AKK |
| 1 | WP_000735335.1 | outer | Vibrio cholerae | 541 | MKKLLPLFVSAALGTLSSAVW |
| | | membrane | | | AENLAEIYNQAKENDPQLLSVA |
| | | channel | | | AQRDAAFEAVTSSRSALLPQIN |
| | | protein TolC | | | LTAGYNINRSDQDPRESDLLSA |
| | | | | | GINFSQELYQRSSWVSLDTAEK |
| | | | | | KARQADSQYAATQQGLILRVA |
| | | | | | KAYFEVLRAQDNLEFVRAEKA |
| | | | | | AVGRQLEQTKQRFEVGLSAITD |
| | | | | | VHDAQAQFDGVLADEVLAENS |
| | | | | | LTNSYEALREITGQEYSKLAVL |
| | | | | | DTKRFAASRTTESSEALIEKAQQ |
| | | | | | QNLSLLAARISQDVARDNISLAS |
| | | | | | SGHLPSLTLDGGYNYGNNSND |
| | | | | | NAKNTSSEEYNDFKIGVNLKVP |
| | | | | | LYTGGNTTSLTKQAEFAYVAAS |
| | | | | | QDLEAAYRSVVKDVRAYNNNI |
| | | | | | NASIGALRAYEQAVISAKSALE |
| | | | | | ATEAGFDVGTRTIVDVLDATRR |
| | | | | | LYDANKNLSNARYDYILSVLQL |
| | | | | | RQAIGTLSEQDVMDVNAGLKV |
| | | | | | AKK |
| 1 | WP_038509790.1 | outer | Vibrio | 542 | MKKLLPLFISAALGSLSTNAFA |
| | | membrane | coralliilyticus | | DTLTEIYNQAKENDPTLLSAAA |
| | | channel | | | TRDSAFEAVTSSRATLLPQISLT |
| | | protein TolC | | | AAYDINRGERNSETNDSDAWN |
| | | | | | AAVGFTQELYKRSSWITLDTAE |
| | | | | | KTARRADATYAAAQQALILRV |
| | | | | | ATAYFDVLKAQDNLVFVQAEK |
| | | | | | AAVGRQLEQTKQRFEVGLSAIT |
| | | | | | DVHDAQAQYDSVLADEVLAQN |
| | | | | | DLINSYEGLREITGLEHTNLNIL |
| | | | | | DIARFSASKTDAPVDALVEEAQ |
| | | | | | QKNLSLLSSRIAQDIAKDNISLQ |
| | | | | | SSGHLPSLTLDGNYTLAEQMNS |
| | | | | | TPSSGDFDSDGFNVGLNLSVPL |
| | | | | | YTGGSTTSLTKQAEFDYVAASQ |
| | | | | | DLEATYRSVVKDVRAFNNNISA |
| | | | | | SIGALRAYEQTVVSAKSALEAT |
| | | | | | EAGFDVGTRTIVDVLDSTRNLY |
| | | | | | DANKNLSNARYDYILSVLQLRQ |
| | | | | | AVGTLSEQDILDINAGLKEAPA |
| | | | | | NTANS |
| 1 | WP_020332188.1 | outer | Vibrio fluvialis | 543 | MKKLLPLLISATLGGLSTTASA |
| | | membrane | | | DDLAQIYDQAKQNDPQLLSAA |
| | | channel | | | AQRDAAFEAINSSRSSLLPQINL |
| | | protein TolC | | | TAGYNINRSDVDLRDSDKLSAG |
| | | | | | INFSQELYDRSSWVSLDTAEKQ |
| | | | | | ARQADAQYANSQQSLMLRVAQ |
| | | | | | AYFDVLSAQDNLEFVRAEKAA |
| | | | | | VGRQLEQTKQRFEVGLSAITDV |
| | | | | | HDAQAQYDTVLADEVLAENSLI |
| | | | | | NSYESLREITGQEHTNLSILDTN |
| | | | | | RFSTSRTTESMEALIEQAQEKNL |
| | | | | | SLLSARISQDVAKDNISLASSGH |
| | | | | | LPSLTLDGGYNYGREYNDNYSS |
| | | | | | YDTYHENNDFNIGLNLTVPLYS |
| | | | | | GGNVSSQTKQAEYAYVAASQD |
| | | | | | LEASYRSVVKNVRAYNNNINAS |
| | | | | | IGSVRAYEQSVISAQSALDATEA |
| | | | | | GFDVGTRTIVDVLDATRTLYSV |
| | | | | | KKNLSDARYSYIINVLQLRQAV |
| | | | | | GTLSEQDIIDVNAGLKAIK |
| 1 | WP_077679956.1 | outer | Vibrio kanaloae | 544 | MKKLLPLFISAAIGSLSSSAFAD |
| | | membrane | | | TLAEVYDQAKQNDPQLLRSAA |
| | | channel | | | QRDAAFEAVTSSRSDLLPQINLT |
| | | protein TolC | | | ANYDINRGDRDTSGTGSSSIDN |
| | | | | | NTWGAAIGFTQELYQRSSWITL |
| | | | | | DTAEKTARQFDSAYAAEQQALI |
| | | | | | LRVSTAYFEVLRAQDNLEFVRA |
| | | | | | EKAAVARQLEQTKQRFEVGLS |
| | | | | | AITDVHDAQAQYDGVLADEVL |
| | | | | | AENDLTNSYEGLREITGQEHAN |
| | | | | | LSILDTDRFSASKSSDSAVALVE |
| | | | | | QAEQKNLSLLAARISQDVAKDN |
| | | | | | ISLASSGHLPSLTLDGSYSLSDQ |
| | | | | | SNSSQNYDQDNLNLGLNLVVPL |
| | | | | | YTGGNTTSLTKQAEYNYVAAS |
| | | | | | EDLEATYRSVVKDVRAFNNNIS |
| | | | | | ASIGALRAYEQSVVSAKSALEA |
| | | | | | TEAGFDVGTRTIVDVLDSTRRL |
| | | | | | YDANKNLSDARYNYILSVLQLR |
| | | | | | QAVGTLSEQDIVDVNAGLKVA |
| | | | | | SK |
| 1 | WP_061051590.1 | outer | Vibrio mimicus | 545 | MKKLLPLFVSAALGTLSSAVW |
| | | membrane | | | AENLAEIYNQAKDNDPQLLSVA |
| | | channel | | | AQRDAAFEAVTSSRSTLLPQINL |
| | | protein TolC | | | TAGYNVNRSDQDPRESDLFSAG |
| | | | | | INFSQELYQRSSWVTLDTAEKK |
| | | | | | ARQADSEYAATQQSLILRVSKA |
| | | | | | YFEVLRAQDNLEFVRAEKAAV |
| | | | | | GRQLEQTKQRFEVGLSAITDVH |
| | | | | | DAQAQYDGVLADEVLAENNLT |
| | | | | | NSYETLREITGQEYSKLSVLDTK |
| | | | | | RFAASRTTESTDALIEQAQQKN |
| | | | | | LSLLSARISQDVARDNISLASSG |
| | | | | | HLPSLTLDGGYNYGNNSNDSA |
| | | | | | KGTSGEEYNDFKIGVNLSVPLY |
| | | | | | SGGNTTSLTKQAEFAYVAASQD |
| | | | | | LEAAYRSVVKDVRAYNNNINA |
| | | | | | SIGALRAYEQAVISAKSALEATE |
| | | | | | AGFDVGTRTIVDVLDATRRLYD |
| | | | | | ANKNLSNARYDYILSVLQLRQA |
| | | | | | IGTLSEQDIMDVNAGLKVAKK |
| 1 | WP_054823010.1 | outer | Vibrio owensii | 546 | MKKLLPLFISAALGSMSTSAWA |
| | | membrane | | | DSLAEIYDLAKQNDPQLLSVAA |
| | | channel | | | QRDQAFEAINSSRSALLPQINLT |
| | | protein TolC | | | AGYDIARGDTDFDSKLRSKQSN |
| | | | | | DQNVLSAGVGFSQELYNRASWI |
| | | | | | TLDTAEKTARRADATYAATQQ |
| | | | | | GLILRVSQAYFEVLRAQDNLVF |
| | | | | | VRAEKAAVGRQLEQTKQRFEV |
| | | | | | GLSAITDVHDAQAQYDAVLAD |
| | | | | | EVLAENDLINSYESLREITGQEH |
| | | | | | KNLNVLDTARFSASRSSEPAEA |
| | | | | | LIEEAKTTNLSLLSARIAQDIAR |
| | | | | | DNISLASSGHLPTLSLDGGYNYS |
| | | | | | DTAESTADNTTDGFNVGVNLA |
| | | | | | VPLYTGGRTTSEVKQAEFGYVS |
| | | | | | ASEDLEAEYRSVVKEVRAQNN |
| | | | | | NINASIGALKAYEQSVVSAQSA |
| | | | | | LEATEAGFDVGTRTIVDVLDAT |
| | | | | | RRLYDANKNLSDARYNYILAG |
| | | | | | LQLRQAVGTLSEQDILDVDAGL |
| | | | | | KPVK |
| 1 | WP_089139236.1 | outer | Vibrio rumoiensis | 547 | MKKLLPLIISLTLGGIHLAHADD |
| | | membrane | | | LAEIYNQAKENDPQLLRSKADR |
| | | channel | | | DAAFEAIESSRGNLLPQINLTAG |
| | | protein TolC | | | YNINRNINAHNSDPISISHNATE |
| | | | | | QNAFTAGINVSQELYRRDSWV |
| | | | | | NLDIAEQNARQQDSAYAAEQQ |
| | | | | | AMILRVSQAYFDVLQAQDNLTF |
| | | | | | IQAEKKAVGRQLEQTKQRFEVG |
| | | | | | LSAITDVHDAQAQYDSVLADEI |
| | | | | | LAKNDVINSYEALREITGQPHK |
| | | | | | DLSVLDTQRFSANVPRTDQNAL |
| | | | | | VDEAQQKNLDLLTARISQDIAK |
| | | | | | SNISLAQSGYTPSLTLDGGYQYS |
| | | | | | DIDEHDDHSATRNYTSNDYNIG |
| | | | | | VNLVVPLYQGGTTNADVKTAQ |
| | | | | | YQYVSASQQLEATYRNVVKNV |
| | | | | | RAYYNNINASIGSIRAYEQSVVS |
| | | | | | AKSALEATEAGYEVGTRTIVDV |
| | | | | | LDSTRRLYDANRQLSDARYSY |
| | | | | | VISVLQLKQAVGTLTEQDILDIN |
| | | | | | AGLKRPS |
| 1 | WP_153446102.1 | outer | Vibrio sp. | 548 | MKKLLPLIISLALGGVTTAHAD |
| | | membrane | SM1977 | | DLAQIYNQAKENDPQLLRSKAD |
| | | channel | | | RDSAFAAIEASRGDLLPQINLTA |
| | | protein TolC | | | GYNINRNINAHNSDPLNISSLAN |
| | | | | | EDNAFTAGVNFSQELYRRDSW |
| | | | | | VNLDIAEQNARQQDSAYAAEQ |
| | | | | | QNLILRTSQAYFGVLQAQDSLT |
| | | | | | FIRAEKKAVARQLEQTKQRFDV |
| | | | | | GLSAITDVHDAQAQYDSVLAD |
| | | | | | EIIGENNVTNSYEELREITGQMN |
| | | | | | KDLALLDTKRFSANSPQTNSNA |
| | | | | | LVDEAQQKNLSLLTARIGQDIA |
| | | | | | KSNISLANSGYSPSLTLDGGYQ |
| | | | | | YSDIDSHDDHSATGNYTSNDYH |
| | | | | | IGVNLIVPLYQGGTTSANVKTA |
| | | | | | EYQYVSASEQLESTYRGVVKN |
| | | | | | VRAYYNNINASIGTIRAYEQSVI |
| | | | | | SAKSALEATEAGYDVGTRTIVD |
| | | | | | VLDATRRLYDANSQLSDARYN |
| | | | | | YILSVLQLKGAVGTLSEQDVLD |
| | | | | | INAGLIKTAATK |
| 1 | WP_011078695.1 | outer | Vibrio vulnificus | 549 | MKKLLPLLIGAALGSLSSSVWA |
| | | membrane | | | DSLAEIYDLAKQNDPQLLSVQA |
| | | channel | | | KRDAAFEAVTSSRSTLLPQINLT |
| | | protein TolC | | | AGYNLKRGDTDLDAGATIDND |
| | | | | | QNALTAGINFSQELYQRSSWITL |
| | | | | | DNAEKTARQADAAYAATQQGL |
| | | | | | ILRTAQAYFEVLKAQDNLEFVR |
| | | | | | AEKAAVARQLEQTKQRFEVGL |
| | | | | | SAITDVHDAQAQYDGVLADEV |
| | | | | | LAENSLTNSYEALREITGQEHK |
| | | | | | NLNVLDTKRFSASRSNASAETLI |
| | | | | | EEAQEKNLSLLSARITKDIAKDN |
| | | | | | ISLASSGHLPSLTLDGGYNYAD |
| | | | | | VSNSAQSDGTTNNFNVGVNLV |
| | | | | | VPLYTGGNTTSQTKQAEFNYVS |
| | | | | | ASQDLEATYRGVVKEVRAQNN |
| | | | | | NINASIGALRAYEQSVVSARSA |
| | | | | | LEATEAGFDVGTRTIVDVLDAT |
| | | | | | RRLYDANKNLSNARYNYILSVL |
| | | | | | QLRQAVGTLSEQDVLDVDAGLI |
| | | | | | AKK |
| 1 | WP_007967744.1 | MULTISPECIES: | Xanthomonas | 550 | MIRRSLVLALAAALSPMAAHAT |
| | | TolC | | | DLLQVYEMARNGDPQLAVAES |
| | | family outer | | | TRLVNREGQVQARAALLPQLD |
| | | membrane | | | GSAGYTQSHRELEGVDGRSTTK |
| | | protein | | | QRQYAIQGSQTIFNWAQFSNLR |
| | | | | | AQREVAKAADFTLASANNDLIT |
| | | | | | RTSAAYFQVLVGIESLAAAETN |
| | | | | | EAAAKKQFDYADKRLEVGLAPI |
| | | | | | TDVHEARAQYDQARADTITAR |
| | | | | | NTLKDYYQALTELTGQPVVGL |
| | | | | | RALPDEFRPEVPAAYSNVDQLV |
| | | | | | ATAIADNPALKAQQLQVSAAE |
| | | | | | AQVSAARAGHLPTLSLGANVG |
| | | | | | RSNSWGGQGTVDEAAGNFTTA |
| | | | | | GRNIDTDSVGITLTIPIFAGGATQ |
| | | | | | SAVRQALSQRDIQQDTYEQQKR |
| | | | | | ALDRNTRNAYQTVVAGISEVEA |
| | | | | | RRLAVVSAQAAYDASQVGLEV |
| | | | | | GTRTVLDVVQNQRTLFQAQLN |
| | | | | | YAQSRYNFLQNRLLLGQAIGKL |
| | | | | | DITDLQDVNRLLSQDAESKLQG |
| | | | | | SGSLQ |
| 1 | WP_016849107.1 | MULTISPECIES: | Xanthomonas | 551 | MIRRSLVLALAAALSPMAVHAT |
| | | TolC | | | DLLQVYEMARNGDPQLAVAES |
| | | family outer | | | TRLVNREGQVQARAALLPQLD |
| | | membrane | | | GSAGYTQSHRELEGVDGRSTTK |
| | | protein | | | QRQYAIQGSQTIFNWAQFSNLR |
| | | | | | AQREVAKAADFTLASANNDLIT |
| | | | | | RTSAAYFQVLVGIESLAAAETN |
| | | | | | EAAAKKQFDYADKRLEVGLAPI |
| | | | | | TDVHEARAQYDQARADTITAR |
| | | | | | NTLKDYYQALTELTGQPVVGL |
| | | | | | RALPDEFRPEVPAAYSNVDQLV |
| | | | | | ATAIADNPALKAQQLQVSAAE |
| | | | | | AQVSAARAGHLPTLSLGANVG |
| | | | | | RSNSWGGQGTVDEAAGNFTTA |
| | | | | | GRNIDTDSVGITLTIPIFAGGATQ |
| | | | | | SAVRQALSQRDIQQDTYEQQKR |
| | | | | | ALDRNTRNAYQTVVAGISEVEA |
| | | | | | RRLAVVSAQAAYDASQVGLEV |
| | | | | | GTRTVLDVVQNQRTLFQAQLN |
| | | | | | YAQSRYNFLQNRLLLGQAIGKL |
| | | | | | DITDLQDVNRLLSQDAESKLQG |
| | | | | | SGSLQ |
| 1 | WP_022558516.1 | MULTISPECIES: | Xanthomonas | 552 | MIRRSLVLALAAALSPMAAHAT |
| | | TolC | | | DLLQVYEMARNGDPQLAVAES |
| | | family outer | | | TRLVNREGQVQARAALLPQLD |
| | | membrane | | | GSAGYTQSHRELEGVDGRSTTK |
| | | protein | | | QRQYAIQGSQTIFNWAQFSNLR |
| | | | | | AQREVAKAADFTLASANNDLIT |
| | | | | | RTSAAYFQVLVGIESLAAAETN |
| | | | | | EAAAKKQFDYADKRLEVGLAPI |
| | | | | | TDVHEARAQYDQARADTITAR |
| | | | | | NTLKDYYQALTELTGQPVVGL |
| | | | | | RALPDEFRPEVPAAYSNVDQLV |
| | | | | | ATAIADNPALKAQQLQVSAAE |
| | | | | | AQVSAARAGHLPTLSLGANVG |
| | | | | | RSNSWGGQGTVDEAAGNFTTA |
| | | | | | GRNIDTDSVGITLTIPIFAGGATQ |
| | | | | | SAVRQALSQRDIQQDTYEQQKR |
| | | | | | ALDRNTRNAYQTVVAGISEVEA |
| | | | | | RRLAVVSAQAAYDASQVGLEV |
| | | | | | GTRTVLDVVQNQRTLFQAQLN |
| | | | | | YAQSRYNFLQNRLLLGQAIGKL |
| | | | | | DITDLQDVNRMLSQDAESKLQ |
| | | | | | GSGSLQ |
| 1 | WP_039572634.1 | MULTISPECIES: | Xanthomonas | 553 | MIRRSLVLALAAALSPMAAHAT |
| | | TolC | | | DLLQVYEMARNGDPQLAVAES |
| | | family outer | | | TRLVNREGQVQARAALLPQLD |
| | | membrane | | | GSASYTQSHRELEGVDGRSTTK |
| | | protein | | | QRQYVIQGSQTIFNWAQFSNLR |
| | | | | | AQREVAKAADFTLASANNDLIT |
| | | | | | RTSAAYFQVLVGIESLAAAETN |
| | | | | | EAAAKKQFDYADKRLEVGLAPI |
| | | | | | TDVHEARAQYDQARADTITAR |
| | | | | | NTLKDYYQALTELTGQPVVGL |
| | | | | | RALPDEFRPEVPAAYSNVDQLV |
| | | | | | ATAIADNPALKAQQLQVSAAE |
| | | | | | AQVSAARAGHLPTLSLGANVG |
| | | | | | RSNSWGGQGTVDEAAGNFTTA |
| | | | | | GRDIDTDSVGITLTIPIFAGGATQ |
| | | | | | SAVRQALSQRDIQQDTYEQQKR |
| | | | | | ALDRNTRNAYQTVVAGISEVEA |
| | | | | | RRLAVVSAQAAYDASQVGLEV |
| | | | | | GTRTVLDVVQNQRTLFQAQLN |
| | | | | | YAQSRYNFLQNRLLLGQAIGKL |
| | | | | | DITDLQDVNRLLSQDAESKLQG |
| | | | | | SGSLQ |
| 1 | WP_012437435.1 | TolC family | Xanthomonas | 554 | MIRRSLVLALAAALSPMAAHAT |
| | | outer | campestris | | DLLQVYEMARNGDPQLAVAES |
| | | membrane | | | TRLVNREGQVQARAALLPQLN |
| | | protein | | | GAFDYSKAHREIEGQDGRITTSS |
| | | | | | RSSQIQGSQTIFNWSQFSTLRAQ |
| | | | | | REVAKAADFTLESANNDLITRT |
| | | | | | SAAYFQVLVGIESLAAAETNEA |
| | | | | | AAKKQFDYADKRLEVGLAPITD |
| | | | | | VHEARAQYDQARADTINARNT |
| | | | | | LKDYYQALTELTGQPVVGLRA |
| | | | | | LPENFRPEVPAAYSNVDQLVAS |
| | | | | | AIADNPALKAQQLQVSAAEASI |
| | | | | | NAARAGHLPTVNLTGSVGRSNS |
| | | | | | WGTGAAESGVFTTQGRDIDTDS |
| | | | | | IGISVSIPIFAGGATQSAVRQAIS |
| | | | | | QRDIQQDTYEQQKRALDRNTR |
| | | | | | NAYQTVVAGISEVEARRLSVVS |
| | | | | | AQAAYDASQVGLEVGTRTVLD |
| | | | | | VVQNQRTLFQAQQLYAQARYT |
| | | | | | FLQNRLLLAQAIGKIDIAELQDV |
| | | | | | NRLLSVEAEAKLQGSGSLQ |
| 1 | WP_011257798.1 | TolC family | Xanthomonas | 555 | MIHRSLVLALAAALSPMAANA |
| | | outer | oryzae | | TDLLQVYEMARNGDPQLAVAE |
| | | membrane | | | STRLVNREGQVQARAALLPQL |
| | | protein | | | DGSAGYTQSHRELEGVGRPTTK |
| | | | | | QRQYAIQGSQTIFNWAQFSNLR |
| | | | | | AQREVTRAADFTLASANNDLM |
| | | | | | TRTSAAYFQVLVGIESLAAAET |
| | | | | | NEAAAKKQFDYADKRLEVGLA |
| | | | | | PITDVHEARAQYDQARADTINA |
| | | | | | RNTLKDYYQALTELTGQPVVG |
| | | | | | LRALPDEFRPEVPAAYSNVDQL |
| | | | | | VATAIADNPALRAQQLQVSAAE |
| | | | | | AHVSAARAGHLPTLNLSGSMG |
| | | | | | RTNSWGGGSDVDTAAGNFMTN |
| | | | | | NGNIDTNSIGITLSIPIFSGGATQS |
| | | | | | AVRQALSQRDIQQDIYEQQKRA |
| | | | | | LDRNTRNAYQTVVAGISEVEAR |
| | | | | | RLAVVSAQAAYDASQVGLEVG |
| | | | | | TRTVLDVVQNQRTLFQAQLNY |
| | | | | | AQSRYTFLQNRLLLGQAIGKLD |
| | | | | | ITDLQDVNRLLSQDAESKLQGS |
| | | | | | GSLQ |
| 1 | WP_053501775.1 | TolC family | Xanthomonas | 556 | MIRRSLVLALAAALSPMAANAT |
| | | outer | oryzae | | DLLQVYEMARNGDPQLAVAES |
| | | membrane | | | TRLVNREGQVQARAALLPQLD |
| | | protein | | | GSAGYTQSHRELEGVGRPTTKQ |
| | | | | | RQYAIQGSQTIFNWAQFSNLRA |
| | | | | | QREVARAADFTLASANNDLMT |
| | | | | | RTSAAYFQVLVGIESLAAAETN |
| | | | | | EAAAKKQFDYADKRLEVGLAPI |
| | | | | | TDVHEARAQYDQARADTINAR |
| | | | | | NTLKDYYQALTELTGQPVVGL |
| | | | | | RALPDAFRPEVPAAYSNVDQLV |
| | | | | | ATAIADNPALRAQQLQVSAAEA |
| | | | | | QVSAARAGHLPTLNLSGSMGR |
| | | | | | TNSWGGGSGVDTAAGNFMTN |
| | | | | | NGNIDTNSIGVTLSIPIFSGGATQ |
| | | | | | SAVRQALSQRDIQQDTYEQQKR |
| | | | | | ALDRNTRNAYQTVVAGISEVEA |
| | | | | | RRLAVVSAQAAYDASQVGLEV |
| | | | | | GTRTVLDVVQNQRTLFQAQLN |
| | | | | | YAQSRYTFLQNRLLLGQAIGKL |
| | | | | | DITDLQDVNRLLSQDAESKLQG |
| | | | | | SGSLR |
| 1 | WP_075239650.1 | TolC family | Xanthomonas | 557 | MIHRSLVLALAAALSPMAANA |
| | | outer | oryzae | | TDLLQVYEMARNGDPQLAVAE |
| | | membrane | | | STRLVNREGQVQARAALLPQL |
| | | protein | | | DGSAGYTQSHRELEGVGRPTTK |
| | | | | | QRQYAIQGSQTIFNWAQFSNLR |
| | | | | | AQREVTRAADFTLASANNDLM |
| | | | | | TRTSAAYFQVLVGIESLAAAET |
| | | | | | NEAAAKKQFDYADKRLEVGLA |
| | | | | | PITDVHEARAQYDQARADTINA |
| | | | | | RNTLKDYYQALTELTGQPVVG |
| | | | | | LRALPDEFRPEVPAAYSNVDQL |
| | | | | | VATAIADNPALRAQQLQVSAAE |
| | | | | | AHVSAARASHLPTLNLSGSMGR |
| | | | | | TNSWGGGSDVDTAAGNFMTN |
| | | | | | NGNIDTNSIGITLSIPIFSGGATQS |
| | | | | | AVRQALSQRDIQQDIYEQQKRA |
| | | | | | LDRNTRNAYQTVVAGISEVEAR |
| | | | | | RLAVVSAQAAYDASQVGLEVG |
| | | | | | TRTVLDVVQNQRTLFQAQLNY |
| | | | | | AQSRYTFLQNRLLLGQAIGKLD |
| | | | | | ITDLQDVNRLLSQDAESKLQGS |
| | | | | | GSLQ |
| 1 | WP_149621362.1 | TolC family | Xanthomonas | 558 | MIRRSLVLALAAALSPMAANAT |
| | | outer | oryzae | | DLLQVYEMARNGDPQLAVAES |
| | | membrane | | | TRLVNREAQVQARAALLPQLD |
| | | protein | | | GSAGYTQSHRELEGVGRSTTKQ |
| | | | | | RQYAIQGSQTIFNWAQFSNLRA |
| | | | | | QREVARAADFTLASANNDLMT |
| | | | | | RTSAAYFQVLVGIESLAAAETN |
| | | | | | EAAAKKQFDYADKRLEVGLAPI |
| | | | | | TDVHEARAQYDQARADTINAR |
| | | | | | NTLKDYYQALTELTGQPVVGL |
| | | | | | RALPDAFRPEVPAAYSNVDQLV |
| | | | | | ATAIADNPALRAQQLQVSAAEA |
| | | | | | QVSAARAGHLPTLNLSGSMGR |
| | | | | | TNSWGGGSGVDTAAGNFMTN |
| | | | | | NGNIDTNSIGVTLSIPIFSGGASQ |
| | | | | | SAVRQALSQRDIQQDTYEQQKR |
| | | | | | ALDRNTRNAYQTVVAGISEVEA |
| | | | | | RRLAVVSAQAAYDASQVGLEV |
| | | | | | GTRTVLDVVQNQRTLFQAQLN |
| | | | | | YAQSRYTFLQNRLLLGQAIGKL |
| | | | | | DITDLQDVNRLLSQDAESKLQG |
| | | | | | SGSLQ |
| 1 | WP_010364366.1 | TolC family | Xanthomonas | 559 | MIRRSLVLALAAALSPMAAHAT |
| | | outer | vasicola | | DLLQVYEMARNGDPQLAVAES |
| | | membrane | | | TRLVNREGQVQARAALLPQLD |
| | | protein | | | GSAAYTQSHRELEGIDGRSTTK |
| | | | | | QRQYAIQGSQTIFNWAQFSNLR |
| | | | | | AQREVAKAADFTLASANNDLIT |
| | | | | | RTSAAYFQVLVGIESLAAAETN |
| | | | | | EAAAKKQFDYADKRLEVGLAPI |
| | | | | | TDVHEARAQYDQARADTINAR |
| | | | | | NTLKDYYQALTELTGQPVVGL |
| | | | | | RALPEEFRPEVPAAYSNVDQLV |
| | | | | | ATAIADNPALKAQQLQVSAAE |
| | | | | | AQVSAARAGHLPTLNLSGSLGR |
| | | | | | TNSWGGGTDVDTAAGNFTTNN |
| | | | | | RNIDTDSIGITLSIPIFAGGATQS |
| | | | | | AVRQALSQRDIQQDTYEQQKR |
| | | | | | ALDRNTRNAYQTVVAGISEVEA |
| | | | | | RRLAVVSAQAAYDASQVGLEV |
| | | | | | GTRTVLDVVQNQRTLFQAQLN |
| | | | | | YAQSRYNFLQNRLLLGQAIGKL |
| | | | | | DITDLQDVNRLLSQDAESKLQG |
| | | | | | SGSLQ |
| 1 1 | WP_010376759.1 | TolC family | Xanthomonas | 560 | MIRRSLVLALAAALSPMAAHAT |
| | | outer | vasicola | | DLLQVYEMARNGDPQLAVAES |
| | | membrane | | | TRLVNREGQVQARAALLPQLD |
| | | protein | | | GSAAYTQSHRELEGIDGRSTTK |
| | | | | | QRQYAIQGSQTIFNWAQFSNLR |
| | | | | | AQREVAKAADFTLASANNDLIT |
| | | | | | RTSAAYFQVLVGIESLAAAETN |
| | | | | | EAAAKKQFDYADKRLEVGLAPI |
| | | | | | TDVHEARAQYDQARADTINAR |
| | | | | | NTLKDYYQALTELTGQPVVGL |
| | | | | | RALPEEFRPEVPAAYSNVDQLV |
| | | | | | ATAIADNPALKAQQLQVSAAE |
| | | | | | AQVSAARAGHLPTLNLSGSLGR |
| | | | | | TNSWGGGTDVDTAAGNFTTNN |
| | | | | | RNIDTDSIGITLSIPIFAGGATQL |
| | | | | | AVRQALSQRDIQQDTYEQQKR |
| | | | | | ALDRNTRNAYQTVVAGISEVEA |
| | | | | | RRLAVVSAQAAYDASQVGLEV |
| | | | | | GTRTVLDVVQNQRTLFQAQLN |
| | | | | | YAQSRYNFLQNRLLLGQAIGKL |
| | | | | | DITDLQDVNRLLSQDAESKLQG |
| | | | | | SGSLQ |
| 1 | WP_039434376.1 | TolC family | Xanthomonas | 561 | MIRRSLVLALAAALSPMAAHAT |
| | | outer | vasicola | | DLLQVYEMARNGDPQLAVAES |
| | | membrane | | | TRLVNREGQVQARAALLPQLD |
| | | protein | | | GSAAYTQSHRELEGIDGRSTTK |
| | | | | | QRQYAIQGSQTIFNWAQFSNLR |
| | | | | | AQREVAKAADFTLASANNDLIT |
| | | | | | RTSAAYFQVLVGIESLAAAETN |
| | | | | | EAAAKKQFDYADKRLEVGLAPI |
| | | | | | TDVHEARAQYDQARADTINAR |
| | | | | | NTLKDYYQALTELTGQPVVGL |
| | | | | | RALPEEFRPEVPAAYSNVDQLV |
| | | | | | ATAIADNPALKAQQLQVSAAE |
| | | | | | AQVSAARAGHLPTLSLGANVG |
| | | | | | RSNSWGGQGTVDEAAGNFTTA |
| | | | | | GRDIDTDSVGITLTIPIFAGGATQ |
| | | | | | SAVRQALSQRDIQQDTYEQQKR |
| | | | | | ALDRNTRNAYQTVVAGISEVEA |
| | | | | | RRLAVVSAQAAYDASQVGLEV |
| | | | | | GTRTVLDVVQNQRTLFQAQLN |
| | | | | | YAQSRYNFLQNRLLLGQAIGKL |
| | | | | | DITDLQDVNRLLSQDAESKLQG |
| | | | | | SGSLQ |
| 1 | WP_004084655.1 | TolC family | Xylella fastidiosa | 562 | MIRRCLALAVTTALFPVVSQAT |
| | | outer | | | NLLQVYEMARAADPQLSAAES |
| | | membrane | | | TRLYSVEGQVQARAALLPQMS |
| | | protein | | | GAATLARSRTEYDGISGSAISKN |
| | | | | | RQYSINGSQTLFNWNQFSNLRS |
| | | | | | QRAIAKAADFTLESAKQNLIVR |
| | | | | | SSAAYFSVLIGIESLIAAEANEAS |
| | | | | | AKRQFDYAQKRLEVGLSPITDL |
| | | | | | HEARASYDQARADTITARNTLQ |
| | | | | | DYYQALAEITGQPVTDLRGLPE |
| | | | | | DFRPEVPAKFSNIDQLVTEAISN |
| | | | | | NPSLKAQQLQVNAAESSISAAR |
| | | | | | GEHYPTLSLSGSLGKSKAWGSS |
| | | | | | SGAMSALIPDRDQPISDTNNLQL |
| | | | | | TLSIPLFAGGATQSRVRQAIAQR |
| | | | | | DIQQDNYEQNKRTLNRNTRNA |
| | | | | | YQNMIAGISEVEARRLAVVSAQ |
| | | | | | AALDASQVGLEVGTRTLLDVV |
| | | | | | QNQRILFSVRQAYVQARYNFLQ |
| | | | | | NRLLLSQNSGTLSIEDVQEINQL |
| | | | | | LTADSERKL |
| 1 | WP_004090307.1 | TolC family | Xylella fastidiosa | 563 | MIRRCLALAVTTALFPVVSQAT |
| | | outer | | | NLLQVYEMARAADPQLSAAES |
| | | membrane | | | TRLYSVEGQVQARAALLPQMS |
| | | protein | | | GTATLARSRTEYDGISGSAISKN |
| | | | | | RQYSINGSQTLFNWSQFSNLRS |
| | | | | | QREIAKAADFTLESAKQNLIVRS |
| | | | | | SAAYFSVLIGIESLIAAEANEAS |
| | | | | | AKRQFDYAQKRLEVGLSPITDL |
| | | | | | HEARASYDQARADTITARNTLQ |
| | | | | | DYYQALAEITGQPVTDLRGLPE |
| | | | | | DFRPEVPAKFSNIDQLVTEAISN |
| | | | | | NPSLKAQQLQVNAAESSISAAR |
| | | | | | GEHYPTLSLSGSLGKSKAWGSS |
| | | | | | SGAMSALIPDRNQPISDTNNLQL |
| | | | | | TLSIPLFAGGATQSRVRQAIAQR |
| | | | | | DIQQDNYEQNKRTLNRNTRNA |
| | | | | | YQNMIAGISEVEARRLAVVSAQ |
| | | | | | AALDASQVGLEVGTRTLLDVV |
| | | | | | QNQRILFSVRQAYVQARYNFLQ |
| | | | | | NRLLLSQNSGTLSIEDVQEINQL |
| | | | | | LTADSERKL |
| 1 | WP_010895004.1 | TolC family | Xylella fastidiosa | 564 | MIRRCLALAVTTALFPVVSQAT |
| | | outer | | | NLLQVYEMARAADPQLSAAES |
| | | membrane | | | TRLYSVEGQVQARAALLPQMS |
| | | protein | | | GTATLARSRTEYDGISGSAISKT |
| | | | | | RQYSINGTQTLFNWSQFSNLRS |
| | | | | | QRAIAKAADFTLESAKQNLIVR |
| | | | | | SSAAYFSVLIGIESLIAAEANEAS |
| | | | | | AKRQFDYAQKRLEVGLSPITDL |
| | | | | | HEARASYDQARADTITARNTLQ |
| | | | | | DYYQALTEITGQPVTDLRGLPE |
| | | | | | DFRPEVPAKFSHIDQLVTEAISN |
| | | | | | NPSLKAQQLQVNAAESSISAAR |
| | | | | | GEHYPTLSLSGSLGKSKAWGTS |
| | | | | | SGAMSALIPDRDQPISDTNNLQL |
| | | | | | TLSIPLFAGGATQSRVRQAIAQR |
| | | | | | DIQQDTYEQNKRTLNRNTRNA |
| | | | | | YQNMIAGISEVEARRLAVVSAQ |
| | | | | | AALDASQVGLEVGTRTLLDVV |
| | | | | | QNQRILFSVRQAYVQARYNFLQ |
| | | | | | NRLLLSQNSGTLSIEDVQEINQL |
| | | | | | LTADSERKL |
| 1 | WP_005188309.1 | MULTISPECIES: | Yersinia | 565 | MKKLLPLLIGLSLAGFSTMSQA |
| | | outer | | | ENLLQVYKQARDSNPDLRKSA |
| | | membrane | | | ADRDAAYEKINQARSPLLPQLG |
| | | channel | | | LGAGYTHTNGFRDASDNPDSN |
| | | protein TolC | | | ATSGSLQLTQTIFDMSKWRALT |
| | | | | | LQEKAAGIQDVTFQTNEQALIL |
| | | | | | NTATAYFNVLRAIDSLSYTEAQ |
| | | | | | KQSVYRQLDQTTQRFNVGLVAI |
| | | | | | TDVQNARASYDTVLASEVTAR |
| | | | | | NNLDNALENLRQITGVYYPELA |
| | | | | | SLNVSRLKTDRPQAVANLLKEA |
| | | | | | EKRNLSLLSARLSQDLAREQIKS |
| | | | | | AETGYMPTIDLTASSSVTNTRY |
| | | | | | SGGSPTSQQVNNDAGQNQIGV |
| | | | | | QLSLPLYSGGATNSAVKQAQY |
| | | | | | NFVGASEQLESAHRSVVQTVRS |
| | | | | | SFNNISASISSIKAYEQVVISNQS |
| | | | | | SLDAMEAGYQVGTRTILDVLTA |
| | | | | | TTNLYQSKQQLADARYNYLIN |
| | | | | | QLNIQSALGTLNMDDLMQLNG |
| | | | | | VLDKPVPTSAASLAPDNANQN |
| | | | | | AYANGESAQSAPRATSVSQPAT |
| | | | | | TAPAAARSGNPFRN |
| 1 | WP_025376991.1 | MULTISPECIES: | Yersinia | 566 | MKKLLPLLIGLSLAGFSSMSQA |
| | | outer | | | ENLLQVYKQARDSNPDLRKSA |
| | | membrane | | | ADRDAAYEKINEARSPLLPQLG |
| | | channel | | | LGAGYTHTNGFRDASDNPDSN |
| | | protein TolC | | | ATSGSLQLTQTIFDMSKWRALT |
| | | | | | LQEKTAGIQDVTFQTSEQKLILD |
| | | | | | TATAYFAVLNAIDTLSYTEAQK |
| | | | | | QSVYRQLDQTTQRFNVGLVAIT |
| | | | | | DVQNARASYDTVLADEVTARN |
| | | | | | NLDNALENLRLITGVYYPELAS |
| | | | | | LNVDRLKTERPQAVNNLLKDA |
| | | | | | EKRNLSLLSARLSQDLAREQIKS |
| | | | | | AETGYMPTIDLTASSSVTNTRY |
| | | | | | SGGTPSTQQVNTDAGQNKIGVQ |
| | | | | | LSLPLYSGGATNSAVKQAQYNF |
| | | | | | VGASEQLESAHRSVVQTVRSSF |
| | | | | | NNISASISSIKAYEQVVISNQSSL |
| | | | | | DAMEAGYQVGTRTILDVLTAT |
| | | | | | TNLYQSKQQLANARYTYLINQL |
| | | | | | NIKSALGTLNMNDLMALNGVL |
| | | | | | DKPVPTSATSLAPDNANQNAY |
| | | | | | ANGTSAQAAPRATAVSQPTTAT |
| | | | | | PAATRNGNPFRN |
| 1 | WP_138774755.1 | MULTISPECIES: | Yersinia | 567 | MKKLLPLLIGLSLAGFSTMSQA |
| | | outer | | | ENLLQVYKQARDSNPDLRKSA |
| | | membrane | | | ADRDAAYEKINEARSPLLPQLG |
| | | channel | | | LGAGYSHTNGFRDASSNPDSNA |
| | | protein TolC | | | TSGSLQLTQTIFDMSKWRALTL |
| | | | | | QEKAAGIQDVTFQTNEQALILN |
| | | | | | TATAYFNVLRAIDSLSYTEAQK |
| | | | | | QSVYRQLDQTTQRFNVGLVAIT |
| | | | | | DVQNARASYDTVLADEVTARN |
| | | | | | NLDNALENLRQITGVYYPELAS |
| | | | | | LNVDRLKTKRPEAVANLLKDA |
| | | | | | EKRNLSLLSARLSQDLAREQIKS |
| | | | | | AETGYMPTIDLTASSSVTNTRY |
| | | | | | SGGTPSTQQVNNDAGQNQIGV |
| | | | | | QLSLPLYSGGATNSAVKQAQY |
| | | | | | NFVGASEQLESAHRSVVQTVRS |
| | | | | | SFNNISASISSIKAYEQVVISNQS |
| | | | | | SLDAMEAGYQVGTRTILDVLTA |
| | | | | | TTNLYQSKQQLADARYNYLIN |
| | | | | | QLNIQSALGTLNMNDLMELNG |
| | | | | | VLDKPVPTSATALAPDNANQN |
| | | | | | AYADGTNTQAAPRVTPVSQPST |
| | | | | | TAPARNGNPFRN |
| 1 | WP_002212186.1 | outer | Yersinia pestis | 568 | MKKLLPLLIGLSLAGFSTMSQA |
| | | membrane | | | ENLLQVYKQARDSNPDLRKAA |
| | | channel | | | ADRDAAYEKINEVRSPLLPQLG |
| | | protein TolC | | | LSAGYTHANGFRDASNSPDSNA |
| | | | | | TSGSLKLTQTIFDMSKWRALTL |
| | | | | | QEKAAGIQDVTFQTSEQQLILN |
| | | | | | TATAYFNVLRAIDSLSYTEAQK |
| | | | | | QSVYRQLDQTTQRFNVGLVAIT |
| | | | | | DVQNARASYDTVLAAEVAARN |
| | | | | | NLDNALESLRQITGVYYPELAS |
| | | | | | LNVERLKTQRPDAVNNLLKEA |
| | | | | | EKRNLSLLSARLSQDLAREQIKS |
| | | | | | AETGYMPTVDLTASSSITNTRY |
| | | | | | SGGTPSSQQVNNDSGQNQIGVQ |
| | | | | | FSLPLYSGGATNSAVKQAQYNF |
| | | | | | VGASELLESAHRNMVQTLRSSF |
| | | | | | NNISASISSINAYQQVVISNQSSL |
| | | | | | DAMEAGYQVGTRTILDVLTAT |
| | | | | | TNLYQSKQQLADARYNYLINQ |
| | | | | | LNIKSALGTLNMNDLMALNAV |
| | | | | | LDKPVPTSAAALAPENTTRQTV |
| | | | | | TTPRAQ |
| 1 | WP_038244905.1 | outer | Yersinia ruckeri | 569 | MKKLLPLLIGLSLAGFSTMSQA |
| | | membrane | | | ENLLQVYKQARESNPDLRKSA |
| | | channel | | | ADRDAAFEKINESRSPLLPQLGL |
| | | protein TolC | | | GADYAYSQGFRDSNGVNSNVT |
| | | | | | AGSLTLTQTIFDMSKWRALTLQ |
| | | | | | EKTAGIQDVTFQTSEQQLILNTA |
| | | | | | IAYFNVLRAIDTLSYTEAQKEA |
| | | | | | VYRQLDQTTQRFNVGLVAITDV |
| | | | | | QNARATYDSILAQEVTAQNGV |
| | | | | | DNALENLRQITGVYYPELASLN |
| | | | | | VDRLKTKRPDAVNNLLSEAEK |
| | | | | | RNLTLLSARLSQDLAREQIKLA |
| | | | | | ETGYMPTINATASSKISNNRYSG |
| | | | | | NAVSDQQRSNDAGQNSIGLSFN |
| | | | | | LPLYSGGATNSQVKQAQYGFV |
| | | | | | GASEQLESAHRSVVQTVRSSYN |
| | | | | | NISASISSINAFKQAVISAQSSLD |
| | | | | | ASEAGYQVGTRTILDVLNATTA |
| | | | | | LYNAKQQLANARYDYLINELTI |
| | | | | | KSSLGTLNQNDLMELNGALDK |
| | | | | | PISTSATKVTSESSQSASASSYA |
| | | | | | QTSAQTAPVSQPTATSTRSAGN |
| | | | | | PFRN |

In some embodiments, the outer membrane channel protein comprises an amino acid sequence that has about 70% or greater (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity to any of the amino acid sequences described herein (e.g., the amino acid sequence of E coli K12 TolC). For example, the outer membrane channel protein may contain an amino acid sequence that has about 70% or greater (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity to SEQ ID NOS: 329, 529, or 540. Additional amino acid residues may be present at the N-terminus or C-terminus of any of these sequences (e.g., a starting methionine (“M”) residue at the N-terminus, or a sequence containing a purification tag at the C-terminus).

### E. Gram-Negative Bacterial Host Cells

The gram-negative bacterial cells may be, but are not limited to, Azotobacter, Bordetella, Brucella, Enterobacter, Erwinia, Escherichia, Klebsiella, Paracoccus, Pseudomonas, Proteus, Rhizobia, Salmonella, Serratia, Shigella, Vibrio, Vitreoscilla, or Yersinia cells. Various strains of such bacteria are contemplated for use as described herein. In some embodiments, the gram-negative bacterial cells may be a species used for live bacterial vaccines, e.g., Bordetella bronhiseptica, Brucella abortus, Salmonella enterica, Salmonella typhi, Shigella flexneri, Vibrio cholerae, or Yersinia enterocolitica. In some embodiments, the gram-negative bacterial cell is an E. coli cell, an S. typhi cell, or a V. cholerae cell. In some embodiments bacteria are genetically modified to alter protease, nuclease, or lipid modification enzymes.

Several methodologies are suitable for introducing nucleic acids encoding heterologous peptides, PCATs, membrane fusion proteins, or further components into gram-negative bacterial cells. A variety of expression constructs and vectors can be constructed and employed for expression and secretion of target peptides. Generally, expression vectors include transcriptional and translational regulatory nucleic acid regions operably linked to the nucleic acid encoding the secretion-tagged peptide of interest and secretion system components. The transcriptional and translational regulatory nucleic acid regions will generally be appropriate to the host cell used to express and secrete the target peptide. In general, the transcriptional and translational regulatory sequences may include, e.g., promoter sequences, ribosomal binding sites, transcriptional start and stop sequences, translational start and stop sequences, and enhancer or activator sequences. Typically, the regulatory sequences will include a promoter and/or transcriptional start and stop sequences. Vectors also typically include a polylinker region containing several restriction sites for insertion of foreign DNA. Heterologous sequences (e.g., a fusion tag such as a His tag) can be used to facilitate purification and, if desired, removed after purification. The construction of suitable vectors containing DNA encoding the target peptide to be secreted, replication sequences, regulatory sequences, and phenotypic selection genes can be prepared using standard recombinant DNA procedures. Isolated plasmids, viral vectors, and DNA fragments can be cleaved, tailored, and ligated together in a specific order to generate the desired vectors using known (see, e.g., Sambrook et al., Molecular Cloning: A Laboratory Manual (Cold Spring Harbor Laboratory Press, New York, NY, 2nd ed. 1989)), including those described in more detail below.

In some embodiments, the first nucleic acid and the second nucleic acid are operably linked to an inducible promoter or a constitutive promoter. In some embodiments, the first nucleic acid is operably linked to a first promoter and the second nucleic acid is operably linked to a second promoter. Examples of inducible promoters include, but are not limited to, pBAD (inducible by arabinose); pLac, pLlacO, tac, and 3× Tac (inducible by lactose/IPTG); pTetO, pLtetO, and pLtetO-1 (inducible by anhydrotetracycline); pPrpB (inducible by propionate); PesaR (inducible by N-(3-oxo-hexanoyl)-L-homoserine lactone (3° C.6HSL)); FixK2 and pR_FixK2 (inducible by blue light, 470 nm); and PcpcG2 (inducible by green light, 532 nm). Examples of constitutive promoters include, but are not limited to, the spc ribosomal protein operon promotor Pspc; the beta-lactamase gene promotor Pbla of plasmid pBR322; the PL promoter of phage lambda; the replication control promoters PRNAI and PRNAII of plasmid pBR322; tetracycline resistance gene (tet) promoter of plasmid pBR322; and the P1 and P2 promoters of the rrnB ribosomal RNA operon. In some embodiments, synthetic inducible promoters and/or synthetic constitutive promoters may be employed. For example, constitutive promoter BBa_J23150 (TTTACGGCTAGCTCAGTCCTAGGTATTATGCTAGC; SEQ ID NO:570), based on the E. coli P1 promoter of rrnB ribosomal RNA operon, may be employed

Expression vectors may further contain one or more selectable marker genes to allow the selection of transformed host cells. Selection genes are well known in the art and will vary with the host cell used. Suitable selection genes can include, for example, genes coding for ampicillin and/or tetracycline resistance, which enables cells transformed with these vectors to grow in the presence of these antibiotics.

Nucleic acids encoding a target peptide for secretion, nucleic acids encoding secretion system components, and vectors including such nucleic acids may be introduced into gram-negative bacterial cells or other host cells in a manner suitable for subsequent integration, amplification, and/or expression of the nucleic acids. The method of introduction is largely dictated by the targeted cell type. Exemplary methods include electroporation, heat shock transformation, and bacterial conjugation.

### III. Methods for Peptide Preparation

Also provided herein are methods for preparing peptides. The methods include:
culturing a gram-negative bacterial cell as described herein such that the heterologous peptide is expressed and secreted from the cytosol to the external environment; and
isolating the secreted heterologous peptide, thereby preparing the peptide.

Once host cells are transformed with nucleic acids containing the heterologous peptide of interest and secretion machine components, the host cells are cultured under conditions to express and secret the heterologous peptide of interest.

Several parameters may be used to monitor and control the progress of the culture in terms of cell growth and peptide expression/secretion. Such parameters include, but are not limited to, optical density (OD), dissolved oxygen (DO), pH, nutrient/energy consumption (such as carbon source), accumulation of metabolic by-products (for example, acetic acid), harvest time, and temperature. A threshold parameter may be established to determine the point at which expression of the peptide should be induced, culturing of the bacteria should be stopped, or some other action should be taken. One threshold parameter or a combination of threshold parameters may be used. The parameter or combination of parameters may be monitored at any suitable time intervals in the culture. For example, OD₆₀₀ and nutrient concentrations may be monitored at one-hour, half-hour, or quarter-hour intervals, without limitation.

Any suitable carbon source (e.g., glucose, glycerol, or the like), supplement, or nutrient may be included in the culture in appropriate amounts. Non-limiting examples of compounds that are contemplated for use in culturing gram-negative bacteria as described herein include, but are not limited to, KH₂PO₄, K₂HPO₄, sodium citrate, (NH₄)₂SO₄, MgSO₄, (Na)₂SO₄, CaCl₂, FeSO₄, and combinations thereof. Carbon sources, other nutrients, inducers, and/or other components may be added to cultures in discrete portions or in continuous fashion, as necessary.

Cultures may be incubated at any temperature that permits growth of the cells. Various temperatures at which to incubate the culture associated with abundant growth include, without limitation, 22° C., 28° C., 37° C., or any combination thereof. Cultures may be maintained for any length of time suitable for peptide expression and secretion. Cultures may be grown, for example, from periods of time ranging from 30 minutes, to 4 hours, 8 hours, 12 hours, 18 hours, 24 hours, or longer.

Any suitable fermentation device (or “fermenter”) is contemplated for use in culturing the gram-negative bacterial cells. For example, the fermenter may contain any number of impellers (e.g., Rushton impellers), intakes, and/or measurement probes. In some embodiments, the fermenter is configured to include one, two, or three Rushton impellers and a ring or tube sparger for introduction of air into the fermenter. The use of manual and/or computer-based systems is contemplated, and the fermentation system may interface with a computerized system for monitoring and control of fermentations. In this manner, the system may be fully or partially automated.

### IV. Methods for Peptide Delivery

Also provided herein are methods for delivering a peptide from gram-negative bacterial cytosol to an external environment. The methods include introducing a gram-negative cell as described herein to the external environment, such that the gram-negative bacterial cell expresses and secretes the heterologous peptide from the cytosol to the external environment, thereby delivering the peptide.

### A. Peptide Administration

Any of the gram-negative bacterial cells described herein can be administered in vitro, ex vivo or in vivo. In some embodiments, the external environment comprises a tissue or organ of a human subject or animal subject, for example, a tissue or organ in a human or animal subject. For example, gram-negative bacterial cells according to the present disclosure may be administered to the subject orally, topically, nasally, by pulmonary administration, by injection (e.g., intramuscularly, intracutaneously, or subcutaneously), or by another route. In some embodiments the gram-negative bacterial cells are administered in a pill (e.g., one or more tablets or capsules), or liquid form preparation (e.g., a solution, suspension, or emulsion) containing one or more pharmaceutically acceptable excipients. As used herein, administer or administration refers to the act of introducing, injecting or otherwise physically delivering a substance as it exists outside the body (e.g. gram-negative bacterial cells according to the present disclosure) into a subject, such as by mucosal, intradermal, intravaginal, intravenous, intratumoral, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

Provided herein is a method for treating a disease or disorder associated with or affected by gut microbiota comprising administering to the subject with the disorder, an effective amount of a gram-negative bacteria or a population of gram-negative bacteria described herein. I some embodiments, the gram-negative bacteria expresses a microcin, for example, any microcin described herein.

Diseases that can be treated with any of the gram-negative bacteria or populations of gram-negative bacteria provided described herein include, but are not limited to, diseases that are impacted by the gut microbiota. These include, but are not limited to, obesity, diabetes, heart disease, central nervous system diseases, autoimmune disorders (e.g., rheumatoid arthritis, inflammatory bowel disease, lupus, Sjogren's syndrome, etc.), metabolic disorders, and cancer. In some embodiments, the disease is associated with inflammation For example, in some embodiments, the subject has gut inflammation, and in some such cases the subject has an inflammatory disease (e.g., Crohn's disease, ulcerative colitis, and the like). In some embodiments, gut inflammation can indirectly impact the disease, such as colorectal cancer, obesity, arthritis and neuromuscular conditions. As used herein the term “gut” refers to the entire gastrointestinal tract of a subject. The gastrointestinal (GI) tract is the tract or passageway of the digestive system that leads from the mouth to the anus. The GI tract contains all the major organs of the digestive system, in humans and other animals, including the esophagus, stomach, small intestine, large intestine, and anus.

As used throughout, by subject is meant an individual. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

As used herein the terms treatment, treat, or treating refers to a method of reducing one or more of the effects of the disorder or one or more symptoms of the disorder. Thus in the disclosed methods, treatment can refer to a 10%, 20%, 30%, 40%, 50%, 60%, 70%, 80%, 90%, or 100% reduction in the severity of the disorder, for example, an inflammatory disorder (e.g., gut inflammation). For example, a method for treating an inflammatory disorder is considered to be a treatment if there is a 10% reduction in one or more symptoms of the inflammatory disorder in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disorder.

In the treatment methods described herein, gram-negative bacterial cells, or pharmaceutical composition comprising gram-negative bacterial cells is administered in a therapeutically effective amount. As used herein, the term therapeutically effective amount or effective amount refers to an amount of a composition comprising any of the gram-negative bacterial cells described herein, hat, when administered to a subject, is effective, alone or in combination with additional agents, to treat a disease or disorder either by one dose or over the course of multiple doses. A suitable dose can depend on a variety of factors including the particular gram-negative bacterial cells used and whether they are used concomitantly with other therapeutic agents. Other factors affecting the dose administered to the subject include, e.g., the type or severity of the disease. For example, a subject having Crohn's disease may require administration of a different dosage of a composition comprising gram-negative bacterial cells described herein, than a subject with ulcerative colitis.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a desired level of response in the cells, tissues and/or organs of a subject.

In some embodiments the gram-negative bacterial cell(s) is introduced into a subject to colonize one or more organs of the subject with the gram-negative bacterial cell(s). By “colonize” is meant that an introduced gram-negative bacterial cell (e.g., a population of bacterial cells) can establish a population of a desired abundance or level, or can establish a large enough population in the target organ, e.g., the gut, of the subject, that the population is detectable, despite the presence of already established bacterial populations. In some cases, the introduced bacteria can reach an abundance, for example, from about 10² CFU/μl to 10¹² CFU/μl (e.g., 10² CFU/μl, 10³ CFU/μl, 10⁴ CFU/μl, 10⁵ CFU/μl, 10⁶ CFU/μl, 10⁷ CFU/μl, 10⁸ CFU/μl, 10⁹ CFU/μl, 10¹⁰ CFU/μl, 10¹¹ CFU/μl, 10¹² CFU/μl) or more, one day, two days, three days, four days, five days, six days, seven days or longer after introduction.

In some cases, the introduced bacteria can reach, for example, an abundance of 10⁵ CFU/μl or more (e.g., 10⁶ CFU/μl or more, 10⁷ CFU/μl or more, 10⁸ CFU/μl or more, 10⁹ CFU/μl or more, or 10¹⁰ CFU/μl or more), one day, two days, three days, four days, five days, six days, seven days or longer after introduction. In some cases, the introduced bacteria can reach an abundance, for example, from about 10² CFU/μl to 10⁴ CFU/μl (e.g., 10² CFU/μl, 10³ CFU/μl, 10⁴ CFU/μl) one day, two days, three days, four days, five days, six days, seven days or longer after introduction.

In some embodiments, the introduced gram-negative bacteria reaches an abundance such that it attains a population level of 1%, or more (e.g., 5% or more, 10% or more, 15% or more, 20% or more, 30% or more, 40% or more, 50% or more, 60% or more, 70% or more, 80% or more, or 90% or more) of total CFUs in the target organ, e.g., the gut, one day, two days, three days, four days, five days, six days, seven days or longer after introduction.

In some embodiments, at least a portion of the population of bacterial cells in an organ (e.g., gut) is displaced as a result of colonization with the gram-negative bacterial cells described herein. For example, in some cases 5% or more (e.g., 10% or more, 15% or more, or 20% or more) of the population of bacterial cells in an organ (e.g., gut) is displaced by the gram-negative bacteria. In some cases, the gram-negative bacterial cell and the bacterial cells present in the gut of the subject, prior to administration of the gram-negative bacteria are the same species. In some cases, the gram-negative bacterial cell and the bacterial cells present in the gut of the subject, prior to administration of the gram-negative bacteria are different species.

In some embodiments, the growth of the gram-negative bacteria is modulated (e.g., increased) by administering an energy and/or carbon source to the subject, thus providing the gram-negative bacteria in the subject with an energy source for growth. Growth can be controlled, for example, by adjusting the amount of carbohydrate provided and/or frequency with which the carbohydrate is provided.

In some embodiments, the carbon source (e.g., carbohydrate) is uncommon in the diet of the individual and is either rarely or not consumed by the gut bacteria in the subject or population of subject. In this way, the gram-negative bacteria administered to the subject have preferred access to a resource (e.g., a carbon source), which thereby provides them with a growth advantage over other bacteria in the organ, e.g., the gut, (at least with respect to that resource).

In some embodiments, colonization is stable for a long period of time. Thus, in some cases a gram-bacterial cell of the disclosure becomes entrenched in the gut. The term “entrench” is used herein to refer to a situation in which an introduced species becomes a stable/persistent member of the community into which it was introduced.

In some embodiments, administration to the gut of a subject can be effected by oral administration. Any convenient type of oral administration can be used. For example, oral administration can include delivery via eating (e.g., incorporated into food), drinking (e.g., incorporated into a solution such as drinking water), oral gavage (e.g., using a stomach tube), aerosol spray, tablets, capsules, pills, powders, and the like. In some embodiments, a gram-negative bacterial cell is introduced into an individual (e.g., into the individual's gut) by delivery into the individual's colon. Any convenient number of gram-negative bacterial cells can be introduced. For example, in some cases 10³ or more cells (e.g., 10⁴ or more, 10⁵ or more, 10⁶ or more, 10⁷ or more, 10⁸ or more cells, 10⁹ or more, of 10¹⁰ or more) cells are introduced. In some cases 10¹¹ or more cells are introduced. In some cases, between 10⁷ and 10¹³ cells are introduced (e.g., between 10⁸-10¹², 10⁹-10¹², or 10¹⁰-10¹² cells).

Formulations for administration will commonly comprise a suspension of the bacterial cells in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The quantity of gram-negative bacterial cells in these formulations can vary widely, and will be selected primarily based on fluid volumes, viscosities, body weight, and the like, in accordance with the particular mode of administration selected and the patient's needs.

Oil suspensions can be formulated by suspending cells in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. These formulations can be preserved by the addition of an antioxidant such as ascorbic acid. The pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. Such formulations can also contain a demulcent, a preservative, or a coloring agent.

### B. Environmental Delivery

In some embodiments, the external environment is an agricultural environment. Gram-negative bacterial cells may be introduced to crops such as Allium, Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Rosa, Secale, Senecio, Sinapis, Solanum, Solanaceae, Sorghum, Trigonella, Triticum, Vitis, Vigna, or Zea.

Provided herein are gram negative ephiphytic or soil bacterial cell(s) for agricultural applications. Cultures and populations comprising any of the ephiphytic or soil bacterial cells described herein are also provided. As used throughout, the term “epiphytic bacteria” refers to bacteria which live on the surface of a plant, for example, on the surface of leaves, roots, flowers, buds, seeds and fruit. The soil bacterial cells described herein can be any bacteria found in soil, for example, a root-associated bacteria, such as, for example, Rhizobium, Bradyrhizobium, Azorhizobium, Allorhizobium, Sinorhizobium and Mesorhizobium.

Provided herein is a gram-negative epiphytic or soil bacterial cell(s) comprising a first nucleic acid encoding a secretion signal sequence fused to a heterologous agricultural peptide, a second nucleic acid encoding a C39 peptidase-containing ATP-binding cassette transporter (PCAT), and a third nucleic acid encoding a membrane fusion protein; and an inner membrane surrounding cytosol, an outer membrane surrounding the inner membrane, and periplasmic space between the inner membrane and the outer membrane; wherein the gram-negative bacterial cell secretes the heterologous agricultural peptide from the bacterial cytosol to an external environment outside of the outer membrane.

A variety of heterologous agricultural peptides can be expressed and secreted by the gram-negative epiphytic bacterial cells or soil bacterial cells of the present disclosure. Typically, the heterologous agricultural peptide will contain from about 5 amino acid residues to about 150 amino acid residues. The heterologous peptide may contain, for example, 5-15 amino acid residues, or 15-25 amino acid residues, or 25-35 amino acid residues, or 35-45 amino acid residues, or 45-55 amino acid residues, or 55-65 amino acid residues, or 65-75 amino acid residues, or 75-85 amino acid residues, or 85-95 amino acid residues, or 95-105 amino acid residues, or 105-115 amino acid residues, or 115-125 amino acid residues, or 125-135 amino acid residues, or 135-145 amino acid residues, or 145-150 amino acid residues.

In some embodiments, the heterologous agricultural polyeptide is an antifungal peptide, an antibacterial peptide, a plant hormone or a plant growth regulator. In some embodiments, the heterologous agricultural peptide comprises a peptide set forth in Table 9. The agricultural peptides provided herein can be prepared by culturing a gram-negative epiphytic or soil bacterial described herein using, for example, the culturing methods described above, such that the heterologous agricultural peptide is expressed and secreted from the cytosol to the external environment; and isolating the secreted heterologous agricultural peptide, thereby preparing the peptide

TABLE 9
Agricultural peptides

| Peptide | Function | Target | Seq |
|---|---|---|---|
| Cm-p5 | antifungal | Candida | SRSELIVHQRLF |
| | | albicans | |
| Drosomycin-2 | antifungal | N. crassa, G. | DCLSGKYKGPCAVWDNEMCRRIC |
| | | candidum, S. | KEEGHISGHCSPSLKCWCEGC |
| | | cerevisiae | |
| anAFP | antifungal | yeasts | SKYGGECSVEHNTCTYLKGGKDHI |
| | | | VSCPSAANLRCKTERHHCEYDEHH |
| | | | KTVDCQTPV |
| alo-3 | antifungal | C. albicans, C. | CIKNGNGCQPNGSQGNCCSGYCHK |
| | | glabrata | QPGWVAGYCRRK |
| AFP | antifungal | Botrytis cinerea | ATYNGKCYKKDNICKYKAQSGKTA |
| | | | ICKCYVKKCPRDGAKCEFDSYKGK |
| | | | CYC |
| AnAFP | antifungal | Aspergillus | LSKYGGECSLEHNTCTYRKDGKNH |
| | | | VVSCPSAANLRCKTDRHHCEYDDH |
| | | | HKTVDCQTPV |
| PAF | antifungal | Botrytis cinerea | AKYTGKCTKSKNECKYKNDAGKD |
| | | | TFIKCPKFDNKKCTKDNNKCTVDT |
| | | | YNNAVDCD |
| PAFB | antifungal | | LSKFGGECSLKHNTCTYLKGGKNH |
| | | | VVNCGSAANKKCKSDRHHCEYDE |
| | | | HHKRVDCQTPV |
| miPEP164a | Leaf | Arabidopsis | MPSWHGMWLLPYWKHTHASTHTH |
| | development | thaliana | THNIYGCACELVFH |
| miPEP165a | Vascular | Arabidopsis | MRWKLFOLRGMLSGSRIL |
| | development | thaliana | |
| miPEP319a | Leaf and | Arabidopsis | MNIHTYHHLLFPSLVFHOSSDVPNA |
| | flower | thaliana | LSLHIHTYEYIIWWIDPFRITLAFR |
| | development | | |
| miPEP858a | Root | Arabidopsis | MGGIESLLFTIVRDIGRYGTVCVVY |
| | growth | thaliana | NIKCVYTTRTKASTRTSHP |
| | promotion | | |
| miPEP171b | Flower | Medicago(medick) | MLLHRLSKFCKIERDIVYIS |
| | development | | |
| miPEP172c | Nodulation | Soybean | MWVLCLFCWPTYTHGS |
| miPEP167c | Nodulation | Soybean | MKGVHHFFHHKYVGLRG |
| miPEP156a | Flower | Brassica | MFCSIQCLGRHLFPLHVREIKKATK |
| | development | (Cabbage) | AIKKGKTL |
| miPEP396a | Increase | Vitis(Grape | MLFHSFLELLFHLPN |
| | pigment | vine) | |
| | (anthocyanin) | | |
| miPEP171d1 | Adventitious | Vitis(Grape | MGYGTTPFITCKMGYGTTP |
| | root | vine) | |
| | formation | | |
| miPEP164c | Increase | Vitis(Grape | MEKQGTCITSSCTTNQ |
| | pigment | vine) | |
| | (anthocyanin) | | |
| PSEP1 | Stimulates | Physcomitrella | MVQPLLARLASAAEFVALPGAILVA |
| | cell growth | patens(earthmoss) | YFSTSRSTEPKRDHRK |
| | and aging | | |
| PSEP18 | Impact on | Physcomitrella | MQAFTDTQGYSSFNGPATTAATTPP |
| | growth rate | patens(earthmoss) | EVVGEFGGKGWRPSS |
| PSEP25 | Impact on | Physcomitrella | MVQSKQGLSLLKFIPKVIRPQTSDV |
| | growth rate | patens(earthmoss) | SSAVLWGTTAACGALWLVQPFDWI |
| | and leafy | | KEQITGPKEESK |
| | shoots | | |
| PSEP3 | Impact on | Physcomitrella | MVHQDNSGSGLRSFNHPNPPPNNN |
| | growth rate | patens(earthmoss) | RPPSNPPVVRNPSSGRTPHPYPPPPH |
| | and | | NYNGYPN |
| | filament | | |
| | branching | | |

In some embodiments, the secretion signal sequence is a microcin secretion signal sequence. In some embodiments, the secretion signal sequence comprises a sequence M-X₁-Xₘ-[B]-Xₙ-G-[J],
M is methionine,
G is glycine,
each residue “X” is independently any amino acid,
subscript 1 is an integer ranging from 0 to 9, subscript m is 2, and subscript n is 9,
residue “B” is isoleucine or leucine, and
residue “J” is alanine or glycine, wherein residue “J” is fused to the heterologous agricultural peptide.

In some embodiments, the secretion signal sequence is an N-terminal sequence:

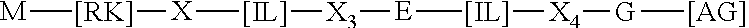

wherein:
M is methionine,
G is glycine,
[RK] is arginine or lysine,
[IL] is isoleucine or leucine,
[E] is glutamic acid,
[AG] is alanine or glycine, and
each residue “X” is independently any amino acid.

The epiphytic or soil bacteria described herein can comprise any of the secretion signal sequences described herein. In some embodiments, the gram-negative epiphytic bacterial cell comprises a signal sequence selected from the group consisting of MKELNLIEVEQVSGA (SEQ ID NO: 673), MKELNKVEVEQVSGA (SEQ ID NO: 674), MRELTSVEMQNVSGA (SEQ ID NO. 675), MRELKTNEIDGVSGG (SEQ ID NO: 676), MRELTSYELQAVSGG (SEQ ID NO: 677), and MRELNVMEVEAVSGA (SEQ ID NO: 678).

In some embodiments, the gram-negative epiphytic or soil bacterial cell is a Gilliamella, Panteoa, Paraburkholdera, Serratia, Pseudomonas, Rhizobium or Bradyrhizobium cell. In some embodiments, the gram-negative epiphytic or soil bacterial cell is a Gilliamella apicola or a Gilliamella apis cell. In some embodiments, the gram-negative epiphytic or soil bacterial cell is a Panteoa vagans cell. In some embodiments, the gram-negative epiphytic or soil bacterial cell is a Paraburkholdera xenovorans cell. In some embodiments, the gram-negative epiphytic or soil bacterial cell is a Serratia plymuthica cell. In some embodiments, the gram-negative epiphytic or soil bacterial cell is a Pseudomonas pituda cell. In some embodiments, the gram-negative epiphytic or soil bacterial cell is a Pseudomonas fluorescens cell. In some embodiments, the negative epiphytic or soil bacterial cell is a Paraburkholdera phytofirmans cell. In some embodiments, the gram-negative epiphytic or soil bacterial cell is a Rhizobium leguminosarum cell. In some embodiments, the gram-negative epiphytic or soil bacterial cell is a Bradyrhizobium japonicum cell.

Any of the PCAT sequences described herein can be used in the epiphytic and soil bacteria provided herein. In some embodiments, the PCAT is selected from the group consisting of a Gilliamella apicola PCAT, a Gilliamella apis PCAT, a Panteoa vagans PCAT, a Paraburkholdera xenovorans PCAT, or a Serratia plymuthica PCAT. In some embodiments, the PCAT can comprise an amino acid sequence selected from the group consisting of SEQ ID NO: 679, SEQ ID NO: 680, SEQ ID NO: 681, SEQ ID NO: 682, and SEQ ID NO: 94.

In some embodiments, the signal sequence and/or the PCAT are from the same bacterial species as the epiphytic bacterial cell. In some embodiments, the signal sequence and/or the PCAT are from different bacterial species. It is understood that the gram-negative epiphytic or soil bacterial cell can endogenously express a peptide secretion system that expresses one or more heterologous agricultural polypeptides, and/or a heterologous peptide secretion system that expresses one or more heterologous agricultural polypeptides.

As used herein, the term “endogenously expresses” or “endogenously expressing” refers to a cell that expresses one or more nucleic acids or peptides as they are found in nature.

As used herein the phrase “heterologous” refers to what is not normally found in nature. The term “heterologous peptide” refers to a peptide not normally found in a given bacterial cell in nature. As such, a heterologous peptide may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell).

Table 10 provides exemplary components of a peptide secretion system that can be used in gram-negative epiphytic or soil bacterial cell to express an agricultural peptide.

TABLE 10
Exemplary epiphytic bacteria comprising components of peptide secretion system

| | | | SEQ ID | | SEQ ID |
|---|---|---|---|---|---|
| Genus | Species | Signal sequence | NO: | PCAT | NO: |
| Gilliamella | apicola | MKELNLIEVEQV | 673 | MIFRKVLQDQSMNRNQFQHILQSL | 679 |
| | | SGA | | HFGWKRKIPQILQTEAAECGLACLT | |
| | | | | MICRYYGMNVDLFSLRNQIGVSSHG | |
| | | | | VTLATLINMSTSVHLISRPLSLDLNEI | |
| | | | | KQLKTPCILHWDLNHFVVLVAVKRS | |
| | | | | KFILHDPAFGRREVGLNEMSKHFSGI | |
| | | | | ALELWPDKEFKPRTERSQLSLIKMLF | |
| | | | | QIDGLKGFLVKILSLSLIIESINLLLPVG | |
| | | | | TQLVMDHVIMAKDHSLLGLICVGLL | |
| | | | | FFILFRTFIAMLRSWSSLIMGSLVDIQ | |
| | | | | WKAGLFGHLMKLPLAYFEKRKLGDI | |
| | | | | QSRFSSLETICKTLTNNIVKSIINILMSI | |
| | | | | GVFIMMFLYGGWLVWIVCGFTLIYII | |
| | | | | LRVATYQRYRQASEEKIVKEAKANS | |
| | | | | HFMESLYGIDTLKSLKLTESRAQYWL | |
| | | | | NMNIDTTNANIRLTKLEMIFSGVNT | |
| | | | | LIMMIEQTSILWIGAALVMDDKMTL | |
| | | | | GMFIAFTAYRAQFSEQAANLINMVL | |
| | | | | DLKMLNLHTERLTDIVLTDTENDIES | |
| | | | | KYSFPKDIPVEFELRDIAYQYDNLSKP | |
| | | | | IFTNINMKIQAGESVAIIGPSGAGKT | |
| | | | | TLMKVMNGLLTPTHGEVLINGIDIYK | |
| | | | | FGVNNYRDIIGSVLQNDKLFAGSISD | |
| | | | | NISSFDTEKNYEWLVECAKYCNIHNE | |
| | | | | IMSMPMGYETLVSELGNSLSGGQK | |
| | | | | QRLLIARALYRKPCLLFLDEATSHLDE | |
| | | | | DNEFTINKAISSLKITRIIIAHRKSTIES | |
| | | | | ADRIVEIGSVNH | |
| Gilliamella | apis | MKELNKVEVEQ | 674 | MIFRKVLQDQSMARNQFQHILQSL | 680 |
| | | VSGA | | HFGWRRKIPQILQTEAAECGLACLT | |
| | | | | MICRYYGMNVDLFSLRNQIGVSSHG | |
| | | | | VTLATLINMSACVQLQSRPLSLDLNE | |
| | | | | INQLKTPCILHWDLNHFVVLVAVKR | |
| | | | | NKFILHDPAFGRREVGINEMSKHFS | |
| | | | | GIALELWPDKEFQPRTQRSQLSLLK | |
| | | | | MLFQIDGLKGFLVKILSLSLIIESINLLL | |
| | | | | PIGTQLVMDHVIIAKDHSLLGLICIGL | |
| | | | | LFFILFRTFIAMLRSWSSLVMESLVDI | |
| | | | | QWKAGLFGHLMKLPLAYFEKRKLG | |
| | | | | DIQSRFVSLEVIRSTLTKSIVSSIINILM | |
| | | | | TIGVFIMMLLYGGWLVWIVCGFTLI | |
| | | | | YIILRVVTYQRYRQASEEKIVKEAKAN | |
| | | | | SHFMESLYGIDTLKSLKLTESRAQYW | |
| | | | | LNMNIDTTNANIRLTKLDMIFNGVN | |
| | | | | TLIMLVEQTLILWVGASLVMNDKM | |
| | | | | TLGMFIAFTAYRSQFSEQAANLINM | |
| | | | | VLDLKMLNLHTERLTDIVLINTENDI | |
| | | | | ESKNQLPKDIPVEFELNNIAYQYDNL | |
| | | | | SQPIFSNINMKIKAGESVAIIGPSGA | |
| | | | | GKTTLMKVMNGLLEPTQGEVLINGI | |
| | | | | DIYKFGVNNYRDIVGSVLQNDKLFA | |
| | | | | GSISDNIASFDNEKNHEWLVECAKH | |
| | | | | CNIHNEIMKMPMGYETLVSELGNSL | |
| | | | | SGGQKQRLLIARALYRKPCLLFLDEA | |
| | | | | TSHLDEDNEAKINNAISSLKITRIIIAH | |
| | | | | RTSTIASADRVIDITSVNH | |
| Panteoa | vagans | MRELTSVEMQN | 675 | MLLLEKLNFKWFNRLPMIRQSQAA | 681 |
| | | VSGAMRELKTN | | ECGLACLGMIANYHGHQIDMITLRR | |
| | | EIDGVSGG | | QFATSLKGATLADVIAMAQQLNMT | |
| | | | | SRALRVEMEELSKLRMPCILHWELN | |
| | | | | HFVVLKKVRGNKITIHDPARGIRELT | |
| | | | | FKEASTAFTGVALELVPSSTFEVKEEK | |
| | | | | ESISMMKLVGSVTGVKSAFAQVLILS | |
| | | | | IALELFGVLGPFFMQWVMDMVLVS | |
| | | | | ADYSLLSLLGVGFIMIALFQTIVTALR | |
| | | | | SWVMSWFSSQLSVQWTINVCHH | |
| | | | | MLKLPLEWFESRHVGDILSRYGSLNT | |
| | | | | IQSTLTSRFISTVLDGVMSIVTVVML | |
| | | | | FIYNAQLAWLVIGLFLAYALLRFMAY | |
| | | | | DPVRRANEEQIISSARTQSSLLETLRG | |
| | | | | IQAVKTNNKQVPRLSAYMNFLVDTT | |
| | | | | NKGIVIQKLNILFGSAQGLLTSVGRV | |
| | | | | VLVWLAALQVLDGNFSAGMLTAFIS | |
| | | | | FSDQFMSRGSGLINAIIDFRMLRMH | |
| | | | | GERLADIVLSETEVSSEGNPGLVSKE | |
| | | | | TDKETDVPQDIKLINLRFRYAPTEPW | |
| | | | | VVEGANLEIKAGESLAIVGPSGQGKT | |
| | | | | TMAKLILGLLHPEEGTMTVGGMDIT | |
| | | | | QTGLEHHRNRIGCVMQDDILFSGSI | |
| | | | | SENISFFDNEPDHAKITRVARLAQIH | |
| | | | | GDIMKMPMNYQSLVGDMGSFLSG | |
| | | | | GQLQRILLARALYREPKILVLDEATSH | |
| | | | | LDIYNEAQINNAIKQMKITRIIIAHRP | |
| | | | | ETIRSADKIVLLNNGKLSEVTAEQLFG | |
| | | | | QTTTHNKTEITHG* | |
| Panteoa | vagans | MRELKTNEIDGV | 676 | MLLLEKLNFKWFNRLPMIRQSQAA | 681 |
| | | SGG | | ECGLACLGMIANYHGHQIDMITLRR | |
| | | | | QFATSLKGATLADVIAMAQQLNMT | |
| | | | | SRALRVEMEELSKLRMPCILHWELN | |
| | | | | HFVVLKKVRGNKITIHDPARGIRELT | |
| | | | | FKEASTAFTGVALELVPSSTFEVKEEK | |
| | | | | ESISMMKLVGSVTGVKSAFAQVLILS | |
| | | | | IALELFGVLGPFFMQWVMDMVLVS | |
| | | | | ADYSLLSLLGVGFIMIALFQTIVTALR | |
| | | | | SWVMSWFSSQLSVQWTINVCHH | |
| | | | | MLKLPLEWFESRHVGDILSRYGSLNT | |
| | | | | IQSTLTSRFISTVLDGVMSIVTVVML | |
| | | | | FIYNAQLAWLVIGLFLAYALLRFMAY | |
| | | | | DPVRRANEEQIISSARTQSSLLETLRG | |
| | | | | IQAVKTNNKQVPRLSAYMNFLVDTT | |
| | | | | NKGIVIQKLNILFGSAQGLLTSVGRV | |
| | | | | VLVWLAALQVLDGNFSAGMLTAFIS | |
| | | | | FSDQFMSRGSGLINAIIDFRMLRMH | |
| | | | | GERLADIVLSETEVSSEGNPGLVSKE | |
| | | | | TDKETDVPQDIKLINLRFRYAPTEPW | |
| | | | | VVEGANLEIKAGESLAIVGPSGQGKT | |
| | | | | TMAKLILGLLHPEEGTMTVGGMDIT | |
| | | | | QTGLEHHRNRIGCVMQDDILFSGSI | |
| | | | | SENISFFDNEPDHAKITRVARLAQIH | |
| | | | | GDIMKMPMNYQSLVGDMGSFLSG | |
| | | | | GQLQRILLARALYREPKILVLDEATSH | |
| | | | | LDIYNEAQINNAIKQMKITRIIIAHRP | |
| | | | | ETIRSADKIVLLNNGKLSEVTAEQLFG | |
| | | | | QTTTHNKTEITHG* | |
| Paraburkholdera | xenovorans | MRELTSYELQAV | 677 | MRFGWKRRLPMMLQTQAAECGLV | 682 |
| | | SGG | | CVGMIANYFGHDMDLASLRRRFTT | |
| | | | | SLKGATLNDIMQMSNQLGLTPRAL | |
| | | | | RLDLNDLAKLNRPCILHWEMNHFV | |
| | | | | VLKEVSRDKITVHDPARGIRDIPMDE | |
| | | | | VSRGFTGVALELMPSASFQPVAEKQ | |
| | | | | SISMLKLIGGVIGIRSAFVQVLLISAAL | |
| | | | | EVCGIISPFYMQWVMDQVLVSADV | |
| | | | | DLLTLLGIGFLMLVVFQNAIAALRSW | |
| | | | | VVTWFSSLLSVQWTASVCSHLLKLP | |
| | | | | MSYFEQRHMGDIVSRFGSVGTIQS | |
| | | | | MLTSRFISTLLDGVMASITLVMLFVY | |
| | | | | SPMLTWLVLGLFAVYALIRWIAYRPL | |
| | | | | RRANEDQIAYSARAQSQLLESIRGV | |
| | | | | QTIKLGNKQEARVATYTNSVVDMT | |
| | | | | NRGIAIQRLSISFSTLQGVISGIGRVV | |
| | | | | LIWLAARQVLQNEFSAGMLVAFISF | |
| | | | | ADQFTSRASGLIDTVIDIRMLRLHGE | |
| | | | | RLADIVLTEPESNMEGSVTRANLVEE | |
| | | | | AKDRAPGIEVSNLRFRYADTEPWVI | |
| | | | | KDCSFTVEPGESVALIGPSGQGKTT | |
| | | | | MAKLLLGLLMPQKGEIRVDGVDIKK | |
| | | | | LGMLGYRDMIGSVMQDDILFAGSI | |
| | | | | ADNISFFDTHPDYAQVERVARIAQI | |
| | | | | HDEITAMPMGYQSLVGDMGSSLSG | |
| | | | | GQAQRVLLARALYRNPSILILDEATS | |
| | | | | HLDVDRERLINDAIRNMEVTRIIIAH | |
| | | | | RPETIRSADRIIAMQQGVAHELRAQ | |
| | | | | QAGEPSTTFAAGRLDSGNVKITYP | |
| Serratia | plymuthica | MRELNVMEVEA | 678 | MDFSHKLNLSFRQKLPLLRQTQAAE | 94 |
| | | VSGA | | CGLSCVGMIAGYYGHSIDMVTLRQ | |
| | | | | RFPTSLKGSTLSDVMSIAQNMGMS | |
| | | | | CRAVRLELSELQKLSLPCVLHWDMN | |
| | | | | HFVVLKSIAGNQVVIHDPARGIRKVP | |
| | | | | MEEVSRSFTGIALELYPVASFEIRDEK | |
| | | | | KSISMLSLIRNVSGIGSAFWQVALLS | |
| | | | | LVLEFFGIISPFYMQWVIDQVLVSAD | |
| | | | | HELLTLLGVAFIGITLFQNIISALRAW | |
| | | | | VTTWFSSMLSVQWSSNLCTHLLGLP | |
| | | | | LSYFEERHVGDILSRFGSISNIQNTLT | |
| | | | | GSFISSIFDGIMSLVTLMVIFTYNATL | |
| | | | | SFVVIGLFLSYAVIRWVSFEPFRQAN | |
| | | | | EDHLMAAALAQSQLLESIRGVQAIK | |
| | | | | LNNKQDLRVSAYANEMVEAANKGI | |
| | | | | TIQKLSIGFNTLQSIISGIGKILLIWLAA | |
| | | | | VQVLEGNFTSGMLIAFITFSDQFLSR | |
| | | | | SSGLINALIEFKMLRLHGERIADIVLT | |
| | | | | EKEKNMESKVTLPEDNGPVRLDVRA | |
| | | | | LSFRYSSTDTAIFSDFNLSIQAGESVAI | |
| | | | | IGPSGQGKTTLAKLLLGLLKPESGTICI | |
| | | | | NDIDHTKLGMSLYRDLIGSVMQSD | |
| | | | | MLFAGSIMDNINFFDANLDRRHVE | |
| | | | | RVARIAQIHDDIMAMPMGYNSMV | |
| | | | | GDMGSSLSGGQIQRVILARALYRNP | |
| | | | | RILILDEATSHLDVARESAINNAIKH | |
| | | | | MDMTRILIAHRPETILSADRIIQINRH | |
| | | | | GAMEIEKSDFIRLVNASQPILHAS* | |

In some embodiments, the gram-negative epiphytic or soil bacterial cell is a Gilliamella apicola cell, wherein the signal sequence is MKELNLIEVEQVSGA (SEQ ID NO: 673), and wherein the PCAT comprises SEQ ID NO: 679.

In some embodiments, the gram-negative epiphytic or soil bacterial cell is a Gilliamella apis cell, wherein the signal sequence is a MKELNKVEVEQVSGA (SEQ ID NO: 674), and wherein the PCAT comprises SEQ ID NO: 680.

In some embodiments, the gram-negative epiphytic or soil bacterial cell is a Panteoa vagans cell, wherein the signal sequence is a MRELTSVEMQNVSGA (SEQ ID NO: 675) or MRELKTNEIDGVSGG (SEQ ID NO: 676), and wherein the PCAT comprises SEQ ID NO: 681.

In some embodiments, the gram-negative epiphytic or soil bacterial cell is a Paraburkholdera xenovorans cell, wherein the signal sequence is a MRELTSYELQAVSGG (SEQ ID NO: 677), and wherein the PCAT comprises SEQ ID NO: 682.

In some embodiments, the gram-negative epiphytic or soil bacterial cell is a Serratia plymuthica cell, wherein the signal sequence is a MRELTSYELQAVSGG (SEQ ID NO: 678), and wherein the PCAT comprises SEQ ID NO: 94.

In some embodiments, the first nucleic acid and the second nucleic acid are operably linked to an inducible promoter or a constitutive promoter. In some embodiments, the first nucleic acid is operably linked to a first promoter and the second nucleic acid is operably linked to a second promoter. In some embodiments, the gram-negative epiphytic or soil bacterial cell further comprises a fourth nucleic encoding an outer membrane channel protein.

Any of the gram negative epiphytic or soil bacterial cells provided herein can be used to modulate one or more properties of a plant. Provided herein is a method for delivering a heterologous agricultural peptide from a gram-negative epiphytic or soil bacteria cytosol to a plant, wherein the method comprises contacting the plant with a gram-negative epiphytic or soil bacterial cell described herein, such that the gram-negative epiphytic or soil bacterial cell expresses and secretes the heterologous peptide from the cytosol to the plant, thereby delivering the peptide to the plant. As used herein, the term “plant” includes plant cells, plant protoplasts, plant cell tissue cultures from which plants can be regenerated, plant calli, plant clumps, and plant cells that are intact in plants, or parts of plants such as embryos, pollen, ovules, seeds, leaves, flowers, branches, fruit, kernels, ears, cobs, husks, stalks, roots, root tips, anthers, and the like.

Any of the bacterial cells described herein, including epiphytic bacteria and soil bacteria may be applied to plants manually or in automated fashion, optionally in combination with a carrier such as an aqueous solution as described above. A crop sprayer or other such agricultural application machine may be used. A crop spray may contain a tank carried on a chassis, for trailing behind a tractor or for use as a self-propelled unit having an integral cab and engine. The machine may further include an extending boom which provides a transverse line of uniformly spaced spray nozzles connected by pipes to the tank. Alternatively, bacteria may be injected into target plant tissues.

### V. Examples

### Example 1. Materials and Methods for Engineering and Study of Peptide Secretion in Gram-Negative Bacteria

Bacterial and yeast strains, growth conditions and genetic modification. Bacterial and yeast strains used in this study are listed in Table 11. For plasmid construction and molecular cloning, E. coli C2987 (a specific type of DH5-alpha E. coli) competent cells (New England Biolabs, Cat #C2987I) were used. Peptides of interest were expressed and secreted from E. coli W3110 in broth culture. E. coli W3110, Listeria monocytogenes EGD-e, E. coli Nissle 1917 (EcN), Salmonella enterica CDC 2861-79, Vibrio cholerae CVD103-HgR, and Saccharomyces cerevisiae CMY 740-1D were used for agar diffusion assays. All gram-negative bacteria were grown in lysogeny broth (LB) media at 37° C. with shaking at 220 rpm unless otherwise stated. L. monocytogenes was grown in tryptic soy broth (TSB) media at 37° C. with shaking at 220 rpm. S. cerevisiae was grown in yeast peptone dextrose (YPD) media at 30° C. with shaking at 275 rpm. The following antibiotics were used, as appropriate: carbenicillin 75 μg/mL, kanamycin 50 μg/mL, streptomycin 100 μg/mL, and chloramphenicol 10 μg/mL. To transfer plasmids, heat-shock transformation was performed for E. coli C2987 and W3110. Electroporation was performed for EcN and S. enterica by following a general protocol. Briefly, bacteria were washed twice with water or 10% glycerol and electroporated at 2.5 kV in a 1 mm gap cuvette. Biparental mating was carried out to transfer plasmids into V. cholerae CVD103-HgR. Spontaneous streptomycin-resistant V. cholerae colonies were first isolated by serial streaking of wild-type culture on LB agar plates containing streptomycin (100 μg/mL). Then, the constructed plasmids were introduced into E. coli SM10 by heat-shock transformation, and 100 μL of antibiotic-free overnight culture of the transformed SM10 was mixed with an equal amount of overnight V. cholerae culture on LB agar plates. The plates were incubated at 37° C. for three hours, and freshly grown bacterial lawns were re-streaked on LB plates containing carbenicillin (75 μg/mL) and streptomycin (100 μg/mL) to isolate single V. cholerae colonies containing target plasmids.

TABLE 11
Strains

| Name | Description |
|---|---|
| Escherichia coli DH5α | fhuA2 lac(del) U169 phoA glnV44 Φ80′ lacZ(del)M15 gyrA96 recA1 relA1 endA1 thi-1 hsdR17 |
| | (used for molecular cloning) |
| Escherichia coli W3110 | Wild-type (used for POI and secretion system expression) |
| Escherichia coli SM10 (λpir) | thi thr leu tonA lacY supE recA::RP4-2-Tc::Mu Km λpir (used for biparental mating) |
| Escherichia coli Nissle 1917 | Wild-type, Probiotic strain |
| Salmonella enterica Ty21a | CDC 2861-79 (used for POI and secretion system expression) |
| Vibrio cholerae CVD103-HgR | mutant from clinical isolate 569B (used for POI and secretion system expression) |
| Listeria monocytogenes | Wild-type (Indicator cells for agar dissusion assay) |
| EGD-e | |
| Sacharomyces cerevisea | MATa his3Δ1 leu2-3, 112 trp1-289 ura3-52 bar1::loxP (Indicator cells for agar dissusion assay) |
| CMY 740-1D | |
| SK00 | E. coli W3110, pBAD18-Km/pACYC184 |
| SK01 | E. coli W3110, MccV positive secretion, encoding pSKP00/pSK01 |
| SK02 | E. coli W3110, MccV negative secretion, encoding pSKP00/pACYC184 |
| SK03 | E. coli W3110, MccV protease-deficient secretion, encoding pSKP00/pSK02 |
| SK04 | E. coli W3110, MccV_V5 positive secretion, encoding pSKP01/pSK01 |
| SK05 | E. coli W3110, MccV_V5 negative secretion, encoding pSKP01/pACYC184 |
| SK06 | E. coli W3110, MccV_V5 protease-deficient secretion, encoding pSKP01/pSK02 |
| SK07 | E. coli W3110, G1P1 positive secretion, encoding pSKP02/pSK01 |
| SK08 | E. coli W3110, G1P1 negative secretion, encoding pSKP02/pACYC184 |
| SK09 | E. coli W3110, G1P2 positive secretion, encoding pSKP03/pSK01 |
| SK10 | E. coli W3110, G1P2 negative secretion, encoding pSKP03/pACYC184 |
| SK11 | E. coli W3110, G1P3 positive secretion, encoding pSKP04/pSK01 |
| SK12 | E. coli W3110, G1P3 negative secretion, encoding pSKP04/pACYC184 |
| SK13 | E. coli W3110, G1P4 positive secretion, encoding pSKP05/pSK01 |
| SK14 | E. coli W3110, G1P4 negative secretion, encoding pSKP05/pACYC184 |
| SK15 | E. coli W3110, G1P5 positive secretion, encoding pSKP06/pSK01 |
| SK16 | E. coli W3110, G1P5 negative secretion, encoding pSKP06/pACYC184 |
| SK17 | E. coli W3110, G1P6 positive secretion, encoding pSKP07/pSK01 |
| SK18 | E. coli W3110, G1P6 negative secretion, encoding pSKP07/pACYC184 |
| SK19 | E. coli W3110, G1P7 positive secretion, encoding pSKP08/pSK01 |
| SK20 | E. coli W3110, G1P7 negative secretion, encoding pSKP08/pACYC184 |
| SK21 | E. coli W3110, G1P8 positive secretion, encoding pSKP09/pSK01 |
| SK22 | E. coli W3110, G1P8 negative secretion, encoding pSKP09/pACYC184 |
| SK23 | E. coli W3110, G1P9 positive secretion, encoding pSKP10/pSK01 |
| SK24 | E. coli W3110, G1P9 negative secretion, encoding pSKP10/pACYC184 |
| SK25 | E. coli W3110, G1P10 positive secretion, encoding pSKP11/pSK01 |
| SK26 | E. coli W3110, G1P10 negative secretion, encoding pSKP11/pACYC184 |
| SK27 | E. coli W3110, G2P1 positive secretion, encoding pSKP12/pSK01 |
| SK28 | E. coli W3110, G2P1 negative secretion, encoding pSKP12/pACYC184 |
| SK29 | E. coli W3110, G2P2 positive secretion, encoding pSKP13/pSK01 |
| SK30 | E. coli W3110, G2P2 negative secretion, encoding pSKP13/pACYC184 |
| SK31 | E. coli W3110, G2P3 positive secretion, encoding pSKP14/pSK01 |
| SK32 | E. coli W3110, G2P3 negative secretion, encoding pSKP14/pACYC184 |
| SK33 | E. coli W3110, G2P4 positive secretion, encoding pSKP15/pSK01 |
| SK34 | E. coli W3110, G2P4 negative secretion, encoding pSKP15/pACYC184 |
| SK35 | E. coli W3110, G2P5 positive secretion, encoding pSKP16/pSK01 |
| SK36 | E. coli W3110, G2P5 negative secretion, encoding pSKP16/pACYC184 |
| SK37 | E. coli W3110, G2P6 positive secretion, encoding pSKP17/pSK01 |
| SK38 | E. coli W3110, G2P6 negative secretion, encoding pSKP17/pACYC184 |
| SK39 | E. coli W3110, G2P7 positive secretion, encoding pSKP18/pSK01 |
| SK40 | E. coli W3110, G2P7 negative secretion, encoding pSKP18/pACYC184 |
| SK41 | E. coli W3110, G2P8 positive secretion, encoding pSKP19/pSK01 |
| SK42 | E. coli W3110, G2P8 negative secretion, encoding pSKP19/pACYC184 |
| SK43 | E. coli W3110, G2P9 positive secretion, encoding pSKP20/pSK01 |
| SK44 | E. coli W3110, G2P9 negative secretion, encoding pSKP20/pACYC184 |
| SK45 | E. coli W3110, G2P10 positive secretion, encoding pSKP21/pSK01 |
| SK46 | E. coli W3110, G2P10 negative secretion, encoding pSKP21/pACYC184 |
| SK47 | E. coli W3110, G3P1 positive secretion, encoding pSKP22/pSK01 |
| SK48 | E. coli W3110, G3P1 negative secretion, encoding pSKP22/pACYC184 |
| SK49 | E. coli W3110, G3P2 positive secretion, encoding pSKP23/pSK01 |
| SK50 | E. coli W3110, G3P2 negative secretion, encoding pSKP23/pACYC184 |
| SK51 | E. coli W3110, G3P3 positive secretion, encoding pSKP24/pSK01 |
| SK52 | E. coli W3110, G3P3 negative secretion, encoding pSKP24/pACYC184 |
| SK53 | E. coli W3110, G3P4 positive secretion, encoding pSKP25/pSK01 |
| SK54 | E. coli W3110, G3P4 negative secretion, encoding pSKP25/pACYC184 |
| SK55 | E. coli W3110, G3P5 positive secretion, encoding pSKP26/pSK01 |
| SK56 | E. coli W3110, G3P5 negative secretion, encoding pSKP26/pACYC184 |
| SK57 | E. coli W3110, G3P6 positive secretion, encoding pSKP27/pSK01 |
| SK58 | E. coli W3110, G3P6 negative secretion, encoding pSKP27/pACYC184 |
| SK59 | E. coli W3110, G3P7 positive secretion, encoding pSKP28/pSK01 |
| SK60 | E. coli W3110, G3P7 negative secretion, encoding pSKP28/pACYC184 |
| SK61 | E. coli W3110, G3P8 positive secretion, encoding pSKP29/pSK01 |
| SK62 | E. coli W3110, G3P8 negative secretion, encoding pSKP29/pACYC 184 |
| SK63 | E. coli W3110, G3P9 positive secretion, encoding pSKP30/pSK01 |
| SK64 | E. coli W3110, G3P9 negative secretion, encoding pSKP30/pACYC184 |
| SK65 | E. coli W3110, G3P10 positive secretion, encoding pSKP31/pSK01 |
| SK66 | E. coli W3110, G3P10 negative secretion, encoding pSKP31/pACYC184 |
| SK67 | E. coli W3110, G4P1 positive secretion, encoding pSKP32/pSK01 |
| SK68 | E. coli W3110, G4P1 negative secretion, encoding pSKP32/pACYC184 |
| SK69 | E. coli W3110, G4P2 positive secretion, encoding pSKP33/pSK01 |
| SK70 | E. coli W3110, G4P2 negative secretion, encoding pSKP33/pACYC184 |
| SK71 | E. coli W3110, G4P3 positive secretion, encoding pSKP34/pSK01 |
| SK72 | E. coli W3110, G4P3 negative secretion, encoding pSKP34/pACYC184 |
| SK73 | E. coli W3110, G4P4 positive secretion, encoding pSKP35/pSK01 |
| SK74 | E. coli W3110, G4P4 negative secretion, encoding pSKP35/pACYC184 |
| SK75 | E. coli W3110, G4P5 positive secretion, encoding pSKP36/pSK01 |
| SK76 | E. coli W3110, G4P5 negative secretion, encoding pSKP36/pACYC184 |
| SK77 | E. coli W3110, G4P6 positive secretion, encoding pSKP37/pSK01 |
| SK78 | E. coli W3110, G4P6 negative secretion, encoding pSKP37/pACYC184 |
| SK79 | E. coli W3110, G4P7 positive secretion, encoding pSKP38/pSK01 |
| SK80 | E. coli W3110, G4P7 negative secretion, encoding pSKP38/pACYC184 |
| SK81 | E. coli W3110, G4P8 positive secretion, encoding pSKP39/pSK01 |
| SK82 | E. coli W3110, G4P8 negative secretion, encoding pSKP39/pACYC184 |
| SK83 | E. coli W3110, G4P9 positive secretion, encoding pSKP40/pSK01 |
| SK84 | E. coli W3110, G4P9 negative secretion, encoding pSKP40/pACYC184 |
| SK85 | E. coli W3110, G4P10 positive secretion, encoding pSKP41/pSK01 |
| SK86 | E. coli W3110, G4P10 negative secretion, encoding pSKP41/pACYC184 |
| SK87 | E. coli DH5α, codon optimized G1P6 positive secretion, encoding pSKP42/pSK01 |
| SK88 | E. coli DH5α, codon optimized G1P6 negative secretion, encoding pSKP42/pACYC184 |
| SK89 | E. coli DH5α, G1P6_2X positive secretion, encoding pSKP43/pSK01 |
| SK90 | E. coli DH5α, G1P6_2X negative secretion, encoding pSKP43/pACYC184 |
| SK91 | E. coli DH5α, G3P2 positive secretion, encoding pSKP23/pSK01 |
| SK92 | E. coli DH5α, G3P2 negative secretion, encoding pSKP23/pACYC184 |
| SK93 | E. coli DH5α, G3P2_2X positive secretion, encoding pSKP44/pSK01 |
| SK94 | E. coli DH5α, G3P2_2X negative secretion, encoding pSKP44/pACYC184 |
| SK95 | E. coli W3110, Pediocin PA-1 positive secretion, encoding pSKP45/pSK01 |
| SK96 | E. coli W3110, Pediocin PA-1 negative secretion, encoding pSKP45/pACYC184 |
| SK97 | E. coli W3110, Pediocin PA-1 protease-deficient secretion, encoding pSKP45/pSK02 |
| SK98 | E. coli W3110, α-factor positive secretion, encoding pSKP46/pSK01 |
| SK99 | E. coli W3110, α-factor negative secretion, encoding pSKP46/pACYC184 |
| SK100 | E. coli W3110, α-factor protease-deficient secretion, encoding pSKP46/pSK02 |
| SK101 | E. coli W3110, eglin C positive secretion, encoding pSKP47/pSK01 |
| SK102 | E. coli W3110, eglin C protease-deficient secretion, encoding pSKP47/pSK02 |
| SK103 | E. coli W3110, EGF positive secretion, encoding pSKP48/pSK01 |
| SK104 | E. coli W3110, EGF negative secretion, encoding pSKP48/pACYC184 |
| SK105 | E. coli Nissle 1917, Pediocin PA-1 positive secretion, encoding pSKP49 |
| SK106 | E. coli Nissle 1917, Pediocin PA-1 negative secretion, encoding pSKP50 |
| SK107 | Salmonella enterica Ty21a, Pediocin PA-1 positive secretion, encoding pSKP49 |
| SK108 | Salmonella enterica Ty21a, Pediocin PA-1 negative secretion, encoding pSKP50 |
| SK109 | Vibrio cholerae CVD103-HgR, Pediocin PA-1 positive secretion, encoding pSKP49 |
| SK110 | Vibrio cholerae CVD103-HgR, Pediocin PA-1 negative secretion, encoding pSKP50 |

Construction of plasmids. General and standard techniques in molecular cloning were used to construct plasmids. All plasmids, primers, and gBLOCKs used are listed in Table 12 and Table 13. Primers and gBLOCKs were ordered from Integrated DNA Technologies (IDT). Plasmid pBAD18-Km or pSK00 was used to expresses peptides of interest. Each peptide of interest was conjugated with the MccV signal peptide (CvaC15, MRTLTLNELDSVSGG; SEQ ID NO: 18) at the N-terminus and cloned into pBAD18-Km using SacI and SalI restriction sites. If required, the V5 tag with two Glycine residues (GGGKPIPNPLLGLDST; SEQ ID NO:571) was conjugated at the C-terminus. Plasmid pSK00 was constructed to avoid t avoid routine CvaC15 conjugation. pSK00 is derived from pBAD18-Km, containing CvaC15 sequences excepting the 15th glycine residue. In pSK00, the residue is replaced with alanine to provide an SfoI restriction site. This allows for cloning of a peptide of interest that contains an N-terminal SmaI restriction site and 5′ blunt-end ligation to provide the CvaC15 sequences. Three SfoI sites of pBAD18-Km were deleted by Gibson assembly using primers listed in Table 10. To express CvaA and CvaB, a plasmid pACYC184 was used. pHK22 was used as a template to amplify cvaA and cvaB genes with the promoter of the tetracycline resistance gene (tet) from pBBR322 for constitutive expression of cvaA/cvaB. This plasmid is referred to as pSK01. Plasmid pSK02 was constructed by introducing a point mutation in cvaB of pSK01 to express mutant-type CvaB (C32S). Primers listed in Table 8 were used to amplify a point mutation-containing fragment, which was cloned into cvaB at BsaI and XmaI restriction sites. A broad-host-range vector, pMMB67EH was used to construct pSK03. Amplified cvaA and cvaB were cloned into pMMB67EH at SalI and SphI sites. The plasmids for random synthetic peptides were built by using reduced random codon (NNK) containing primer sets, or gBLCOKs and cognitive primer sets. The gBLOCKs contain nucleotide sequences that are back-translated with codon optimization (ebi.ac.uk/Tools/st/emboss_backtranseq/) from peptide sequences randomly generated by the Sequence Manipulation Suite, version 2 (GenScript Corporation). When NNK codon primers were used, transformed colonies were sequenced to collect peptide sequences that do not have premature stop codons. Other plasmids for peptide expression were constructed by using either codon optimized open reading frame (ORF)-containing gBLOCKs or primers, cognitive primer sets, and the selected plasmids described above.

TABLE 12

| | | SEQ | | |
|---|---|---|---|---|
| | | ID | | |
| Type | Name | NO | Sequence | Usage |
| primer | random 20 mer_ | 614 | ATAGAGCTCGAATTCAGGAGGAAACGATGAGA | template for |
| | V5 | | ACTCTGACTCTAAATGAATTAGATTCTGTTTCTG | generating |
| | | | GTGGTNNKNNKNNKNNKNNKNNKNNKNNKNNK | group2 peitde |
| | | | NNKNNKNNKNNKNNKNNKNNKNNKNNKNNKN | ORFs |
| | | | NKGGAGGAGGTAAACCTATTCCTAATCCTCTCC | |
| | | | TAGGTTTAGATTCTACTTAAGTCGACAGGAGGA | |
| | | | AACGA | |
| primer | cvaC15_induce_ | 615 | ATAGAGCTCGAATTCAGGAGGAAACGATGAGA | amplyfing DNA |
| | F | | ACTCTGACTCTAAATG | containing |
| | | | | CvaC15 |
| | | | | sequences |
| primer | V5_R | 616 | TATGTCGACTTAAGTAGAATCTAAACCTAGGAG | amplyfing DNA |
| | | | AGG | containing V5- |
| | | | | tag sequences |
| primer | cvaB_mutation_ | 617 | CATCAGACGGAGACCGCTGAATCTGGACTG | to make pSK02 |
| | F | | | |
| primer | cvaB mutation_ | 618 | GAGCCCGGGTGGCCATCC | to make pSK02 |
| | R | | | |
| primer | pBAD_vecF | 619 | GTCTGCTTACATAAACAGTAATACAAGGGGTG | to delete Sfo I |
| | | | | site of |
| | | | | pBAD_cvi_cvaC |
| primer | pBAD_vecR | 620 | GCGCCACAGGTGCGGTTGCTATCTCCTTGCTGCC | to delete Sfo I |
| | | | TCGCG | site of |
| | | | | pBAD_cvi_cvaC |
| primer | pBAD_fragF | 621 | GCGCGAGGCAGCAAGGAGATAGCAACCGCACC | to delete Sfo I |
| | | | TGTGG | site of |
| | | | | pBAD_cvi_cvaC |
| primer | pBAD_fragR | 622 | GCTCATAACACCCCTTGTATTACTGTTTATGTAA | to delete Sfo I |
| | | | GCAGACAGTTTTATTGTTCATGA | site of |
| | | | | pBAD_cvi_cvaC |
| gBLOCK | pBAD_MCS_ | 623 | ATAGAGCTCGAATTCAGGAGGAAACGATGAGA | template for |
| | 2 | | ACTCTGACTCTAAATGAATTAGATTCTGTTTCTG | generating |
| | | | GCGCCGGTACCCGGGGATCCTCTAGAGTCGACC | pSK00 (MccV |
| | | | TGCAGGCATGCAAGCTTGGCTGTTTTGGCGGAT | signal peptide, |
| | | | GAGAGAAGATTTTCAGCCTGATACAGATTAAAT | sfoI site |
| | | | CAGAACGCAGAAGCGGTCTGATAAAACAGAAT | containing) |
| | | | TTGCCTGGCGGCAGTAGCGCGGTGGTCCCACCT | |
| | | | GACCCCATGCCGAACTCAGAAGTGAAACGCCGT | |
| | | | AGCGCCGATGGTAGTGTGGACTAGTGGTCTCCC | |
| | | | CATGCATA | |
| primer | pBAD_MCS_ | 624 | TATGCATGGGGAGACCACTAGTCCACACT | to generate |
| | 2_R | | | pSK00 or |
| | | | | amplify the |
| | | | | region |
| gBLOCK | EglinC | 625 | GAGCTCGAATTCAGGAGGAAACGATGAGAACT | to clone eglinC |
| | | | CTGACTCTAAATGAATTAGATTCTGTTTCTGGTG | into pBAD |
| | | | GTACCGAATTTGGCAGCGAACTGAAAAGCTTTC | |
| | | | CGGAAGTGGTGGGCAAAACCGTGGATCAGGCG | |
| | | | CGCGAATATTTTACCCTGCATTATCCGCAGTATG | |
| | | | ATGTGTATTTTCTGCCGGAAGGCAGCCCGGTGA | |
| | | | CCCTGGATCTGCGCTATAACCGCGTGCGCGTGT | |
| | | | TTTATAACCCGGGCACCAACGTGGTGAACCATG | |
| | | | TGCCGCATGTGGGCGGAGGAGGTAAACCTATTC | |
| | | | CTAATCCTCTCCTAGGTTTAGATTCTACTTAAAA | |
| | | | GTCGAC | |
| primer | EglinC_R | 626 | ATAGTCGACTTAGCCCACATGCGGCACATGGT | to amplify |
| | | | | eglinC |
| primer | pBAD_N10 mer | 627 | ATCCCGGGNNKNNKNNKNNKNNKNNKNNKNNK | to generating |
| | | | NNKNNKGGAGGAGGTAAACCTATTCCTAATCCT | group1 peitde |
| | | | CTCCTAGGT | ORFs |
| primer | G3P2_bbs1 | 628 | ATAGAGCTCGAAGACCATGGCGTGCAGGGC | to generate bbsI |
| | | | | site containing |
| | | | | G3P2 |
| primer | G3P2_bbs2 | 629 | ATGAAGACAAGCCATGGTTCTGTTCATAGCTAT | to generate |
| | | | CATCGCGG | G3P2_2X via |
| | | | | SacI and BbsI |
| | | | | cloning |
| gBLOCK | Pediocin PA-1 | 630 | AAATACTACGGCAATGGGGTGACCTGTGGGAAA | template for |
| | | | CATTCCTGCTCCGTTGACTGGGGGAAAGCGACC | Pedicin PA-1 |
| | | | ACCTGTATCATCAATAACGGAGCGATGGCCTGG | |
| | | | GCTACGGGCGGTCACCAGGGCAATCACAAGTGT | |
| primer | Pediocin_F | 631 | ATAGAGCTCAGGAGGAAACGATGAGAACTCTG | amplyfing |
| | | | ACTCTAAATGAATTAGATTCTGTTTCTGGTGGTA | Pedicin PA-1 |
| | | | AATACTACGGCAATGGTGTAACG | |
| primer | Pediocin_F | 632 | ATATCTAGATTAGCACTTATGATTTCCCTGGTGG | amplyfing |
| | | | C | Pedicin PA-1 |
| primer | alpha_F | 633 | ATCCCGGGTGGCACTGGCTGCAGCTGAAACCGG | amplyfing |
| | | | GTCAGCCGATGTACTAAGGTACCTGGGGATCCT | alpha-factor |
| | | | CTAGAG | |
| gBLOCK | EGF | 634 | ATACCCGGGAACAGCGATAGCGAATGCCCGCTG | template for |
| | | | AGCCATGATGGCTATTGCCTGCATGATGGCGTG | EGF |
| | | | TGCATGTATATTGAAGCGCTGGATAAATATGCG | |
| | | | TGCAACTGCGTGGTGGGCTATATTGGCGAACGC | |
| | | | TGCCAGTATCGCGATCTGAAATGGTGGGAACTG | |
| | | | CGCTAAGTCGACATA | |
| primer | EGF_F | 635 | ATACCCGGGAACAGCGATAG | amplyfing EGF |
| primer | EGF_R | 636 | TATGTCGACTTAGCGCAGTTCCC | amplyfing EGF |
| gBLOCK | Random1 | 637 | ACACTTTGCTATGCCCGGGTATATGTGCTGGCAT | template for |
| | | | CAGGCGATTAACGTGATGGAACCGTGCGCGCAG | G3P1 and G4P1 |
| | | | TTTTATCAGGATATTGTGCTGAGCAGCCGCGTG | |
| | | | CAGTGGCAGGATATGGATATGAGCATGCCGCGC | |
| | | | CTGTATAAAATGCAGTATGTGGCGAAAAGCCAT | |
| | | | TTTAGCATTATGTATTTTATTCATCGCGAAGATA | |
| | | | TTCAGAGCAGCGGCTGCTGCGATTGCAACTGCA | |
| | | | ACCCGGTGCGCAAAATTTTTTGCACCCGCTATGT | |
| | | | GGAAATGGTGGATTGCGGCTATTGGCGCCATTT | |
| | | | TTGGCTGCCGCCGGAAGAAGGAGGAGGTAAAC | |
| | | | CTATTCCTAATCCCGGGCTGAGCAACGGCCAGT | |
| | | | GCAACATGCATTGCTGCCCGTGCCTGGAATATC | |
| | | | AGGATTATCATAAACATTATAGCAACACCGAAA | |
| | | | GCTTTAAACAGCTGGTGTGGATGACCCATATTT | |
| | | | GCGATAACTATGCGCTGAGCCATCGCGCGAAAT | |
| | | | GGGGAGGACGTGATGTCTTC | |
| gBLOCK | Random2 | 638 | ACACTTTGCTATGCCCGGGTTTATTATTTATATT | template for |
| | | | CCGAAAGTGTATGATTGGTATGCGGCGAGCATG | G3P2 and G4P2 |
| | | | GGCTGCACCTATCCGGATGGCCTGGGCTTTCGC | |
| | | | ATGGTGAGCATGCGCGTGATTTGGTATGCGTGG | |
| | | | TATGTGTATAGCTGCTATACCGCGGTGGAAACC | |
| | | | AACAAATTTGGCGATTGCGGCTGCGGCAACTAT | |
| | | | AACCATCCGGATCAGGAACAGATTCGCTATAAA | |
| | | | ACCAGCGAACAGGAAAGCACCAAAATGTTTTTT | |
| | | | CGCTGGGCGCCGGAATGGCAGAACAACCACAG | |
| | | | GCACCACCTGATGGTGGAAATGGGAGGAGGTA | |
| | | | AACCTATTCCTAATCCCGGGCATGGCGTGCAGG | |
| | | | GCATTAACATTGAAAAACAGCCGAAACGCAAC | |
| | | | AACCCGGAAAACGAACAGACCCGCATGAAAAT | |
| | | | GCGCCAGGAACGCGATTGGAGCTGCTTTGCGAT | |
| | | | GTTTCATGCGATTACCCGCGATGATAGCTATGA | |
| | | | ACAGAAC | |
| gBLOCK | Random3 | 639 | ACACTTTGCTATGCCCGGGCACAGTTATTGCGA | template for |
| | | | ATGGCTGATGGCGAAAATTCTGAGCATGGGCGA | G3P3 and G4P3 |
| | | | ACAGTGGTGGCATAAATATTATTTTGGCCTGCG | |
| | | | CCATCAGTTTAACGTGAGCAAAGGCTATCATTT | |
| | | | TAACACCAGCTTTGATTTTCATCGCTGCGGCGA | |
| | | | ACCGAAAAACAACTATTATGCGCGCACCCATTG | |
| | | | CGAAAAAGAATATAGCAACGATGATGTGCATCC | |
| | | | GCGCCAGAACAGCATGGGCCGCTGGGGCGCGG | |
| | | | AATGGCCGGCGCTGATTTTTAAACCGCTGTTTG | |
| | | | GCATTAACAAATGGGGCAACTTTGGAGGAGGTA | |
| | | | AACCTATTCCTAATCCCGGGGAAACCTGCCATT | |
| | | | GGATGGAACTGCATATTCCGCTGTTTGAAACCG | |
| | | | ATAGCTTTAAACCGTATGATCCGAAAAGCCTGG | |
| | | | ATAGCGGCCATTGCCTGTATTATGGCTTTTTTTT | |
| | | | TAAATATATTGGCGGCCTGCATGCGATGTGCAT | |
| | | | GTATGGAGGACGTGATGTCTTC | |
| gBLOCK | Random4 | 640 | ACACTTTGCTATGCCCGGGAGCGGCATTTATTG | template for |
| | | | CCAGATTACCCGCTGGGTGCATCCGTTTAGCGA | G3P4 and G4P4 |
| | | | AAGCACCCAGAACATGGATAACATGGCGAACA | |
| | | | CCAAACGCAAACCGCAGTGGCATTATCCGCGCC | |
| | | | GCCATCAGCATAAAGAAGCGCAGTTTGTGTTTT | |
| | | | TTGGCATTGCGCGCGGCTGGCCGTTTCAGTTTTT | |
| | | | TGAACAGGGCTGGGTGACCATTGATGTGCATGA | |
| | | | AGAACATCTGTGGATTTTTTGCTTTGGCGAACGC | |
| | | | AACATGGAACATGGCAACGTGGAACGCGCGCC | |
| | | | GACCATTAGCAGCCGCATTAAAGGAGGAGGTA | |
| | | | AACCTATTCCTAATCCCGGGGTGCAGATGATTT | |
| | | | GGGGCTTTTGCAGCGGCTGGCCGATGATTACCT | |
| | | | GGTATGCGATGATGTTTGCGCATATTCAGTGGG | |
| | | | CGTTTTGGAACACCAAAATTAGCAGCCGCGGCT | |
| | | | GGGAATTTATGGCGAGCGCGTGGCCGGAATATT | |
| | | | TTGTGGGAGGACGTGATGTCTTC | |
| gBLOCK | Random5 | 641 | ACACTTTGCTATGCCCGGGAAACATTGGAAACA | template for |
| | | | GCGCCATGTGTATTATCGCCAGGCGGTGTATCA | G3P5 and G4P5 |
| | | | GCAGCAGAAATGCCAGATGTATAACGTGCCGTA | |
| | | | TAGCCCGGCGAGCGGCTGCTTTAACTGCCAGCC | |
| | | | GAACTGCCATCGCAAAGATTTTGATTGGCGCGA | |
| | | | TCATACCGGCTATTGCTTTAAAATGCTGCAGTTT | |
| | | | CATAACTTTATTACCCCGAAACGCTGCACCGAA | |
| | | | AGCGCGCTGTTTGCGGGCCAGGAATGCCGCCCG | |
| | | | GATTTTGCGCAGGAAAGCGGCGCGCAGAAAGA | |
| | | | TTTTAGCGGCATGCATCCGGGCGGAGGAGGTAA | |
| | | | ACCTATTCCTAATCCCGGGATTATGGAATGCAG | |
| | | | CACCCTGATTTGCACCACCCTGGATTGCTTTATT | |
| | | | TGCGTGCAGGGCCAGTGGCGCTATTGCATGTGG | |
| | | | AACCAGTGCTGGTGCGTGTGCATGAACTGGACC | |
| | | | AAAACCAACTGCGCGCAGTATAGCGTGGCGAA | |
| | | | ACATGGAGGACGTGATGTCTTC | |
| gBLOCK | Random6 | 642 | ACACTTTGCTATGCCCGGGTTTAACCCGGAAAG | template for |
| | | | CAGCCATCAGCCGAGCACCATTCCGAAAAGCCA | G3P6 and G4P6 |
| | | | TTTTCGCATTATTTGCCATTTTGTGCGCGATTGG | |
| | | | CATTTTCCGTGCGGCAGCTGGACCTTTAGCGTG | |
| | | | GTGGATATTTATTTTTGCGAAATGTATACCACCC | |
| | | | TGGGCAACCCGCATGGCTTTGTGATTTGCTGCA | |
| | | | CCTATGGCAGCCAGTATAGCGGCGATAACCGCT | |
| | | | GCGCGGATAAACTGGAACGCCATCCGGCGATGA | |
| | | | TGGAAAACACCTATGGCTGGCATGGCCATACCA | |
| | | | GCGCGGGCCTGGCGCAGCCGGGAGGAGGTAAA | |
| | | | CCTATTCCTAATCCCGGGATTATTTGCTGGTGGC | |
| | | | CGCATCCGCAGTGCTGCTGGAACTTTGAATATT | |
| | | | GCTTTCGCAAAAACTATCTGACCTGCTTTCAGTG | |
| | | | CAGCGAACAGTATAGCACCACCTTTGCGCCGGT | |
| | | | GAACGCGTTTCCGACCATTTGGCAGATTATTTAT | |
| | | | GGAGGACGTGATGTCTTC | |
| gBLOCK | Random7 | 643 | ACACTTTGCTATGCCCGGGGATAAAATGGAACC | template for |
| | | | GCAGTGGAACCATAGCCCGCGCTGCTTTGCGAG | G3P7 and G4P7 |
| | | | CCTGTGCTGCGGCGGCAGCCATACCATGATGGC | |
| | | | GTGGAACCATGTGATTAGCTGGAAAGGCCGCGA | |
| | | | TCTGGTGATTGGCGTGAACCGCCATTGCGCGAC | |
| | | | CCCGCCGCATAGCCAGTTTTGGCATAACGCGTG | |
| | | | GTGGCAGGGCTTTATTAAACATGTGATTGAAAC | |
| | | | CCCGCGCCTGGGCAACATGGCGAAAAACATGCA | |
| | | | GTGCTTTGCGGCGTGCGCGCTGGCGGTGGCGTT | |
| | | | TCCGGTGGATATTAGCCAGCTGGGAGGAGGTAA | |
| | | | ACCTATTCCTAATCCCGGGGGCAGCGTGATTGT | |
| | | | GCTGGATTGGGTGCAGGGCACCAACCTGGTGCA | |
| | | | TAAACAGCATAACACCATTAACCGCCGCCATCA | |
| | | | TCATAGCCATCAGGAAATGTATCCGTGGCCGAC | |
| | | | CGTGTTTGAAATGCATGGGAACCGCATTATTGA | |
| | | | AGAAGGAGGACGTGATGTCTTC | |
| gBLOCK | Random8 | 644 | ACACTTTGCTATGCCCGGGCGCGGCAAATGGAT | template for |
| | | | GATGTGCAACACCCATATGGCGCATTGGAACAC | G3P8 and G4P8 |
| | | | CTATTTTTGGGATGGCAGCGCGGTGCATCTGAC | |
| | | | CGATGATTTTTATCGCAACGGCCCGGCGAAAAG | |
| | | | CTATAACCTGTTTATGGTGCAGAACCATAAAGA | |
| | | | AAGCCGCCATAACTATAAAGTGTGCTTTTTTTGC | |
| | | | TATCTGATTACCACCTATATTACCATTGCGAAAC | |
| | | | ATAAACGCATGAACGAAAACTATTGGTGGATGG | |
| | | | CGCAGGTGTATCTGAAATTTGTGCGCTGGCATG | |
| | | | CGCGCAACTGCTGCTATGCGGGAGGAGGTAAAC | |
| | | | CTATTCCTAATCCCGGGCATGTGCCGAAATGGT | |
| | | | GGTATAAAGGCTTTGATTGGACCACCCAGGTGT | |
| | | | GGCCGTATGCGGCGATGCTGGGCTTTATTAACG | |
| | | | CGCATCATCTGGATACCGTGATGATTAAAATTC | |
| | | | ATCTGGGCGCGTTTCATAACTGCGATTGGGTGG | |
| | | | AAGGAGGACGTGATGTCTTC | |
| gBLOCK | Random9 | 645 | ACACTTTGCTATGCCCGGGGTGATGCAGGGCGA | template for |
| | | | TACCTGGTGGGAATGCGAACCGAGCGAAGCGG | G3P9 and G4P9 |
| | | | AAATTCAGATGCTGTATTGGCCGTGGGGCAGCC | |
| | | | AGAAAGATCCGATTGATTGGGCGTATCTGTGCG | |
| | | | ATACCTGGAAATATACCGGCGATCTGTGCAGCG | |
| | | | GCGGCCCGGAACAGCCGGATGAACATCGCATTC | |
| | | | ATGATGCGATTGGCCGCGCGTTTTATCGCCCGT | |
| | | | GCCCGAGCCTGAACATGTATTATCTGAGCCAGC | |
| | | | GCTGGGCGATTTTTGATACCCATAACAGCCTGG | |
| | | | CGGCGGGCAGCTATTGCTTTATGGGAGGAGGTA | |
| | | | AACCTATTCCTAATCCCGGGGTGACCACCTTTA | |
| | | | AACTGTGGGCGAAAGCGCTGGTGGCGTTTATGT | |
| | | | ATGATGCGGATCATCATGTGAACGATTTTCTGC | |
| | | | CGACCCTGTATCGCGTGTATACCACCATGAACA | |
| | | | TTTGGCATTTTAAACATAAATGCCCGTGCACCA | |
| | | | GCTATGGAGGACGTGATGTCTTC | |
| gBLOCK | Random10 | 646 | ACACTTTGCTATGCCCGGGACCGGCCGCCAGGT | template for |
| | | | GACCGAAGTGACCGTGTGGCATGCGCTGACCAC | G3P10 and |
| | | | CATTTGCGGCATTAGCGAACTGGAATTTACCTA | G4P10 |
| | | | TGGCGCGTGCCCGATGTGGGAAAACATGGAACT | |
| | | | GGAAAAATTTAGCGGCAACGTGTGCTATGAACT | |
| | | | GCAGGATCATTGCTTTTGCGATTGGTGGCAGTA | |
| | | | TACCGAACGCTGCCTGGAAAACCTGCCGATGAT | |
| | | | TGAACTGCCGATTCAGTGGAAACCGTTTACCCT | |
| | | | GCATGAATGGTGGATTATTGGCCGCTGCCCGCT | |
| | | | GACCATTATGAACAGCTGGGCGGGAGGAGGTA | |
| | | | AACCTATTCCTAATCCCGGGGATGATGATGATG | |
| | | | AACTGCTGCGCCTGTGCGATAACATTACCTTTTT | |
| | | | TATGATGTGCATTCATGAATTTACCATGAAACC | |
| | | | GTGGTTTAAAACCATTTGGTTTCTGATGTGCTGG | |
| | | | AACGCGTTTCTGAACCAGGGCAGCAATAGCACC | |
| | | | CATGGAGGACGTGATGTCTTC | |
| primer | 100 mer_F | 647 | ACACTTTGCTATGCCCGGG | amplyfing |
| | | | | group 4 random |
| | | | | peptides |
| primer | 100 mer_R | 648 | GGGATTAGGAATAGGTTTACCTCCTCC | amplyfing |
| | | | | group 4 random |
| | | | | peptides |
| primer | 50 mer_F | 649 | GAGGTAAACCTATTCCTAATCCCGGG | amplyfing |
| | | | | group 3 random |
| | | | | peptides |
| primer | 50 mer_R1 | 650 | AGGAATAGGTTTACCTCCTCCCCATTTCGCGCG | amplyfing |
| | | | ATGGC | G3P1 |
| primer | 50 mer_R2 | 651 | AGGAATAGGTTTACCTCCTCCGTTCTGTTCATAG | amplyfing |
| | | | CTATCATCGCGG | G3P2 |
| primer | 50 mer_R3 | 652 | AGGAATAGGTTTACCTCCTCCATACATGCACAT | amplyfing |
| | | | CGCATGCAG | G3P3 |
| primer | 50 mer_R4 | 653 | AGGAATAGGTTTACCTCCTCCCACAAAATATTC | amplyfing |
| | | | CGGCCACGC | G3P4 |
| primer | 50 mer_R5 | 654 | AGGAATAGGTTTACCTCCTCCATGTTTCGCCACG | amplyfing |
| | | | CTATACTGC | G3P5 |
| primer | 50 mer_R6 | 655 | AGGAATAGGTTTACCTCCTCCATAAATAATCTG | amplyfing |
| | | | CCAAATGGTCGGAAACG | G3P6 |
| primer | 50 mer_R7 | 656 | AGGAATAGGTTTACCTCCTCCTTCTTCAATAATG | amplyfing |
| | | | CGGTTCCCATGC | G3P7 |
| primer | 50 mer_R8 | 657 | AGGAATAGGTTTACCTCCTCCTTCCACCCAATCG | amplyfing |
| | | | CAGTTATGAAA | G3P8 |
| primer | 50 mer_R9 | 658 | AGGAATAGGTTTACCTCCTCCATAGCTGGTGCA | amplyfing |
| | | | CGGGCAT | G3P9 |
| primer | 50 mer_R10 | 659 | AGGAATAGGTTTACCTCCTCCATGGGTGCTATT | amplyfing |
| | | | GCTGCCCT | G3P10 |
| primer | G1P6_opti_F | 660 | ATCCCGGGCTGTCTATGTCTATCTGCATGCGTCC | amplyfing |
| | | | GAAAGGAGGAGGTAAACCTATTCCTA | G1P6 with |
| | | | | E. coli |
| | | | | optimized |
| | | | | codon |
| primer | G1P6_2X_opti_ | 661 | ATCCCGGGCTGTCTATGTCTATCTGCATGCGTCC | amplyfing |
| | F | | GAAACTGTCTATGTCTATCTGCATGCGTCCGAA | G1P6_2X with |
| | | | AGGAGGAGGTAAACCTATTCCTA | E. coli |
| | | | | optimized |
| | | | | codon |
| primer | cvi_cvaC_ | 662 | GGTACCAGGAGGAAACGATGGATAGAAAAAGA | first cloning |
| | pBAD_F | | ACAAAATTAGAGTTGTTATTTGC | occvi and cvaC |
| | | | | into pBAD180- |
| | | | | Km |
| prime | pBAD_cvi_F | 663 | GGTGGTGAATTCAGGAGGAAACGATGGATAGA | amplyfing cvi |
| | | | AA | |
| primer | pBAD_cvi_R | 664 | GCGTGGTACCTCATTTAGAGTCAGAGTTC | amplyfing cvi |
| primer | pBAD_cvaC_ | 665 | GGTGGTGGTACCATGAGAACTCTGACTCTAAAT | amplyfing cvaC |
| | F | | | |
| primer | pBAD_cvaC_ | 666 | GGCGGCGTCGACTCTAGATTATAAACAAACATC | amplyfing cvaC |
| | R | | ACT | |
| primer | No_cvaC15_ | 667 | ATGAGGTACCATGGCTTCAGGGCGTGATATTG | amplyfing cvaC |
| | cvaC_F | | | (no cvaC15) |
| primer | CvaC_V5_R | 668 | CAGGTCGACTTAAGTAGAATCTAAACCTAGGAG | amplyfing |
| | | | AGGATTAGGAATAGGTTTACCTCCTCCTAAACA | cvaC_V5 |
| | | | AACATCACTAAGATTATTTGGACT | |
| primer | CvaAB_F | 669 | ATATCTAGATTTCAGTGCAATTTATCTCTTCAAA | amplyfing |
| | | | TGTAGCACCTGAAGTCAGCCCCATACGATATAA | CvaA/CvaB |
| | | | GTTGTAATTCTCATGTTTGACAGCTTATCATCGA | with pTc |
| | | | TAAGCTTTAATGCGGTAGTTTATCACAGTTAAAT | promoter |
| | | | TGCTAACGCAGTCAGGCACCGTGTAGGAGGAAA | |
| | | | CGATGTTTCGCCAGGATGCTTTAGAAAAC | |
| primer | CvaAB_R | 670 | ATACCTGAGGGTATTATTTAATATAAGAAAGAA | first amplyfing |
| | | | CAGTTATTGGACAATCCAC | CvaA/CvaB |
| | | | | into |
| | | | | pACYC184 by |
| | | | | XbaI, Bsu36I |
| | | | | cloning |
| primer | CvaAB_F2 | 671 | ATAGAGCTCTGGGTACCCGGGGATCCTCTAGAG | amplyfing |
| | | | TCGACAGGAGGAAACGATG | CvaA/CvaB to |
| | | | | construct |
| | | | | pSK03 |
| primer | CvaAB_R2 | 672 | ATAGCATGCCTGCAGTTAAATAGAAATAACTC | amplyfing |
| | | | | CvaA/CvaB to |
| | | | | construct |
| | | | | pSK03 |

*cvaC: encodes Microcin V (MccV)

TABLE 13

| Name | Description | Usage |
|---|---|---|
| pACYC184 | CmR, TetR | Backbone plasmid |
| pBAD18-Km | KanR | Backbone plasmid |
| pMMB67EH | AmpR | Backbone plasmid |
| pBR322 | AmpR | Template |
| pHK11 | AmpR | Template |
| pSK00 | pBAD18-Km derived plasmid, KanR | POI expression |
| pSK01 | pACYC184 derived plasmid, CmR | CvaA/CvaB expression |
| pSK02 | pACYC184 derived plasmid, CmR | CvaA/CvaB C32S expression |
| pSK03 | pMMB67EH derived plasmid, AmpR | POI, CvaA/CvaB expression |
| pSKP00 | pBAD18-Km derived plasmid, KanR | Cvi, MccV expression |
| pSKP01 | pBAD18-Km derived plasmid, KanR | Cvi, MccV_V5 expression |
| pSKP02 | pBAD18-Km derived plasmid, KanR | G1P1 expression |
| pSKP03 | pBAD18-Km derived plasmid, KanR | G1P2 expression |
| pSKP04 | pBAD18-Km derived plasmid, KanR | G1P3 expression |
| pSKP05 | pBAD18-Km derived plasmid, KanR | G1P4 expression |
| pSKP06 | pMMB67EH derived plasmid, AmpR | G1P5 expression |
| pSKP07 | pBAD18-Km derived plasmid, KanR | G1P6 expression |
| pSKP08 | pBAD18-Km derived plasmid, KanR | G1P7 expression |
| pSKP09 | pBAD18-Km derived plasmid, KanR | G1P8 expression |
| pSKP10 | pBAD18-Km derived plasmid, KanR | G1P9 expression |
| pSKP11 | pBAD18-Km derived plasmid, KanR | G1P10 expression |
| pSKP12 | pBAD18-Km derived plasmid, KanR | G2P1 expression |
| pSKP13 | pBAD18-Km derived plasmid, KanR | G2P2 expression |
| pSKP14 | pBAD18-Km derived plasmid, KanR | G2P3 expression |
| pSKP15 | pBAD18-Km derived plasmid, KanR | G2P4 expression |
| pSKP16 | pBAD18-Km derived plasmid, KanR | G2P5 expression |
| pSKP17 | pBAD18-Km derived plasmid, KanR | G2P6 expression |
| pSKP18 | pBAD18-Km derived plasmid, KanR | G2P7 expression |
| pSKP19 | pBAD 18-Km derived plasmid, KanR | G2P8 expression |
| pSKP20 | pBAD18-Km derived plasmid, KanR | G2P9 expression |
| pSKP21 | pBAD18-Km derived plasmid, KanR | G2P10 expression |
| pSKP22 | pBAD18-Km derived plasmid, KanR | G3P1 expression |
| pSKP23 | pBAD18-Km derived plasmid, KanR | G3P2 expression |
| pSKP24 | pBAD18-Km derived plasmid, KanR | G3P3 expression |
| pSKP25 | pBAD18-Km derived plasmid, KanR | G3P4 expression |
| pSKP26 | pBAD18-Km derived plasmid, KanR | G3P5 expression |
| pSKP27 | pBAD18-Km derived plasmid, KanR | G3P6 expression |
| pSKP28 | pBAD18-Km derived plasmid, KanR | G3P7 expression |
| pSKP29 | pBAD18-Km derived plasmid, KanR | G3P8 expression |
| pSKP30 | pBAD18-Km derived plasmid, KanR | G3P9 expression |
| pSKP31 | pBAD18-Km derived plasmid, KanR | G3P10 expression |
| pSKP32 | pBAD18-Km derived plasmid, KanR | G4P1 expression |
| pSKP33 | pBAD18-Km derived plasmid, KanR | G4P2 expression |
| pSKP34 | pBAD18-Km derived plasmid, KanR | G4P3 expression |
| pSKP35 | pBAD18-Km derived plasmid, KanR | G4P4 expression |
| pSKP36 | pBAD18-Km derived plasmid, KanR | G4P5 expression |
| pSKP37 | pBAD18-Km derived plasmid, KanR | G4P6 expression |
| pSKP38 | pBAD18-Km derived plasmid, KanR | G4P7 expression |
| pSKP39 | pBAD18-Km derived plasmid, KanR | G4P8 expression |
| pSKP40 | pBAD18-Km derived plasmid, KanR | G4P9 expression |
| pSKP41 | pBAD18-Km derived plasmid, KanR | G4P10 expression |
| pSKP42 | pBAD18-Km derived plasmid, KanR | codon-optimized GIP6 expression |
| pSKP43 | pBAD18-Km derived plasmid, KanR | G1P6_2X expression |
| pSKP44 | pBAD18-Km derived plasmid, KanR | G3P2_2X expression |
| pSKP45 | pBAD18-Km derived plasmid, KanR | Pediocin PA-1 expression |
| pSKP46 | pBAD18-Km derived plasmid, KanR | α-factor |
| pSKP47 | pBAD18-Km derived plasmid, KanR | Eglin C expression |
| pSKP48 | pBAD18-Km derived plasmid, KanR | EGF expression |
| pSKP49 | pMMB67EH derived plasmid, AmpR | Pediocin PA-1, CvaA/CvaB expression |
| pSKP50 | pMMB67EH derived plasmid, AmpR | Pediocin PA-1 expression |

Calculation of peptide properties. The “Peptides” R package (cran.r-project.org/web/packages/Peptides/index.html) was used to calculate charge and hydrophobicity of random synthetic peptides; calculation methods are described in the Peptides package documentation. After calculation, peptide properties were plotted and grouped. Amino acid frequencies in a particular class were determined per group and normalized by the total number of amino acids in the group (#of certain amino acids/#of total amino acids) to calculate the percentage of the composition.

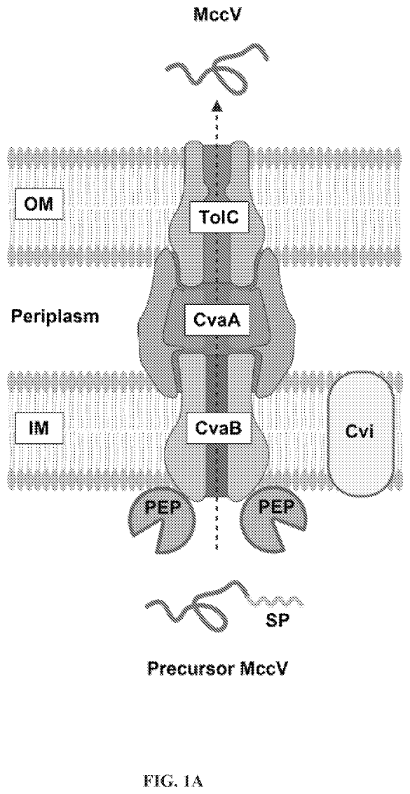

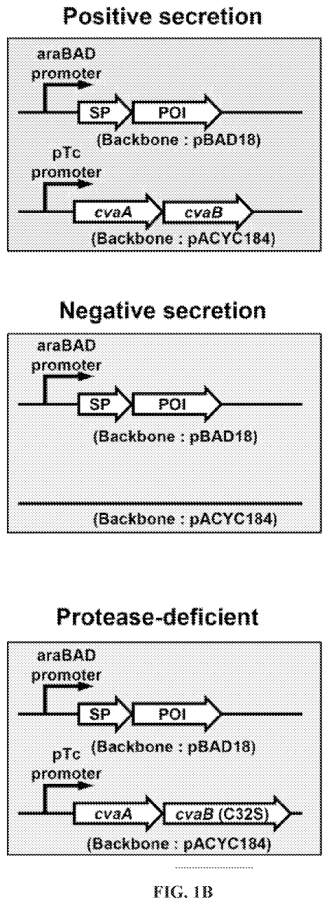

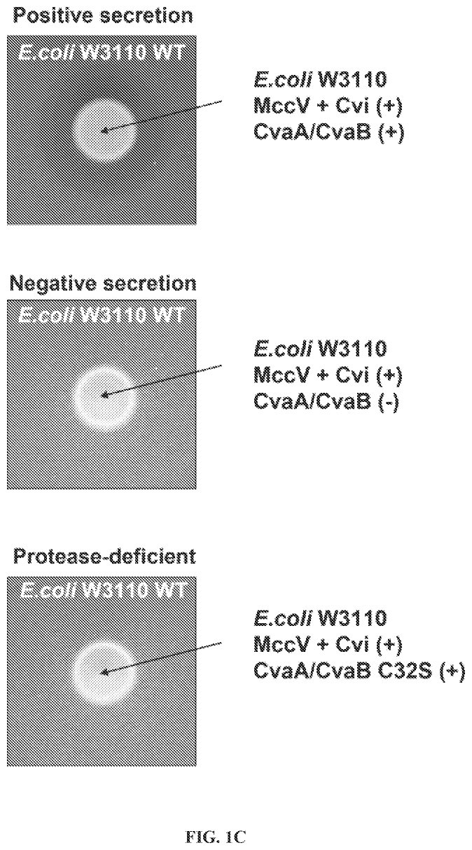

Agar diffusion assay. Indicator strains and testing strains were grown overnight as described above. To observe the zone of inhibition by MccV or MccV_V5, E. coli W3110 wild-type was used as an indicator strain. Overnight culture of the indicator strain was diluted to A₆₀₀=0.001 in 1.5% (w/v) agar-containing LB medium with 0.2% (v/v) arabinose and solidified. The overnight cultures of testing strains, including empty vector, positive secretion and negative secretion (FIG. 1), were centrifuged at 5000 g for 5 min and the pellets were re-suspended in 100 μL of fresh LB medium. 5 μL of the suspensions were spotted on the solidified agar and pictures were taken after overnight incubation at 37° C.

Similarly, L. monocytogenes was used as an indicator strain to detect zone of inhibition by Pediocin PA-1. Overnight L. monocytogenes culture was diluted to A₆₀₀=0.001 in 1.5% (w/v) agar-containing TSB with 0.2% (v/v) arabinose and solidified. Overnight cultures of EcN, S. enterica, and V. cholerae containing either positive or negative secretion were enriched and spotted as described above. Pictures were taken after overnight incubation at 37° C.

S. cerevisiae CMY 740-1D was used as an indicator strain to detect zone of inhibition by α-factor. Overnight culture of the yeast indicator strain was diluted to A₆₀₀=0.01 in 2.0% (w/v) agar-containing yeast peptone glycerol (YPG) media with 0.2% (v/v) arabinose and solidified. The overnight cultures of testing strains were enriched as described above, and 25 μL aliquots were spotted on the solidified YPG agar plate. Pictures were taken after 30 hours incubation at 30° C.

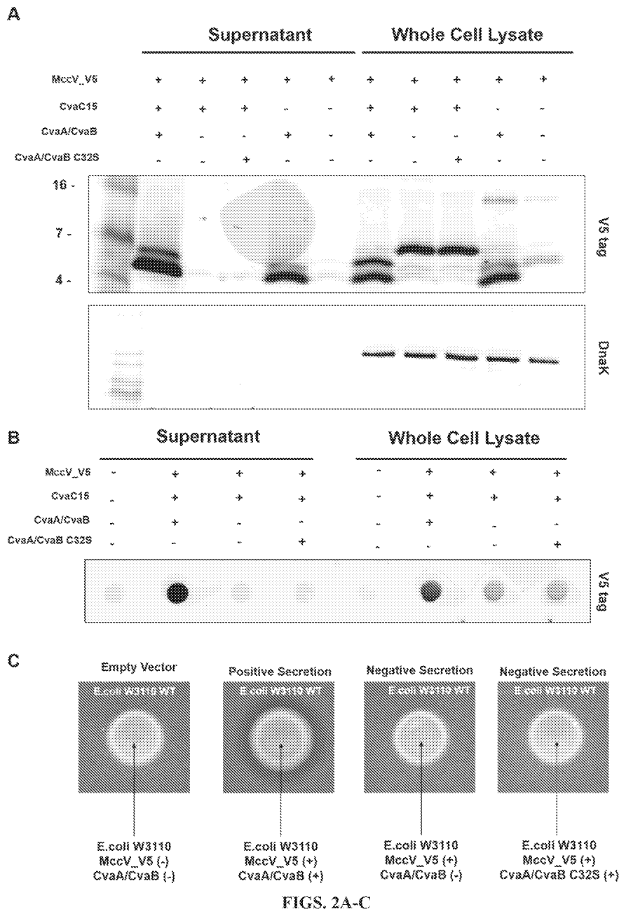

Western blot. To detect MccV_V5 (FIG. 2), overnight E. coli liquid culture was diluted in LB medium to A₆₀₀=0.5. The culture was induced with 0.2% (v/v) arabinose for 2 hours, and normalized to A₆₀₀=1.0. Culture aliquots (250 μL) were centrifuged at 5000 g for 5 min to separate supernatant and cell pellet. The Invitrogen Novex Tricine Gel System (Thermo Fisher Scientific) was used to perform SDS-PAGE. The acquired supernatant and cell pellet were suspended in Tricine SDS Sample Buffer (Cat #LC1676) with Sample Reducing Agent (Cat #NP0009), and 10 μL was loaded into each well of 16% Tricine gel (Cat #EC66952) with 10 μL of SeeBlue Plus2 Pre-stained Protein Standard (Cat #LC5925). Electrophoresis was conducted using Tricine SDS Running Buffer (Cat #LC1675) prior to transfer of peptides to nitrocellulose membrane (Cat #LC2000). The membrane was blocked with 5% low-fat milk in TTBS (50 mM Tris-Cl, pH 7.5, 150 mM NaCl, 0.05% Tween-20 (v/v)) and proteins were labeled by incubation with the selected primary antibody, anti-V5 antibody (Sigma-Aldrich Cat #V8012), or anti-DnaK antibody (Enzo Cat #ADI-SPA-880) 1:5000 diluted in 1% BSA (Bovine Serum Albumin) in TTBS. LICOR IRDye 800CW Goat Anti-Mouse IgG (LI-COR Biosciences, Cat #926-32210) was used as the secondary antibody 1:5000 diluted in 5% low-fat milk in TTBS. The Li-Cor Odyssey Clx Near IR imaging system was used for visualizing. Band intensities were measured using Image Studio software (licor.com/bio/image-studio-lite/download).

Dot blot. E. coli culture samples for dot blot were grown in test tubes using the growth conditions as described above for Western blotting, or in a 96-well deep well plate (Southern Labware Cat #503062). When using the deep well plate, a single colony was inoculated to 1 mL of LB medium in each well and incubated at 37° C. with shaking at 1000 rpm. The plate was sealed by a permeable membrane (Diversified Biotech Cat #BEM-1) for the proper air circulation. After overnight growth, cultures were diluted in 500 μL of fresh LB medium to A₆₀₀=0.5 with 0.2% (v/v) arabinose and incubated at 37° C. with shaking at 1000 rpm for 8 hours. The plate was centrifuged at 4000 rpm for 10 min to collect supernatant samples. A nitrocellulose membrane (GE Healthcare Life Sciences, Cat #10600010) was inserted into 96-well Bio-Dot Apparatus (Bio-rad, Cat #1706545), and 100 μL of each supernatant was loaded onto each well in the apparatus according to the manufacturer's protocol. Two standard synthetic peptides, NPS_V5 (SFRNGVGSGAKKTSFRRAKQGGKPIPNPLLGLDST; SEQ ID NO:572) and ECP_V5 (YRWRCKNQGGGKPIPNPLLGLDST; SEQ ID NO:573) were synthesized by GenScript with ≥90% and ≥95% purity, respectively. Each peptide was dissolved in water to make 1 mg/mL stock solutions, and the solutions were further diluted by a factor of two in LB medium as shown in FIG. 5, and 100 μL of diluent and LB medium were loaded into the wells. To perform the dot blot for whole cell lysate samples, induced cultures were boiled for 20 min and centrifuged at 5000 g for 5 min to separate supernatant and cell debris. 100 μL of the supernatants were loaded onto wells. After samples were transferred to the membrane, the membrane was washed twice with TTBS and removed from the apparatus. Blocking, antibody incubation, visualization and signal intensity calculation were conducted as described above for the Western blotting procedure.

Elastase inhibition assay. Eglin C positive and negative secretion (C32S) E. coli W3110 were grown overnight, and 100-fold dilutions were made in Fresh LB medium. Once the cultures reached A₆₀₀=0.5, the medium was replaced with M9 minimal medium with 0.2% (v/v) arabinose and grown overnight for induction. Cell-free supernatants were collected by centrifuging the cultures at 5000 g for 5 min and filtering using PES 0.22 μm filter membranes (Celltreat Scientific, Cat #229747). Protease inhibition activity of the samples was tested using Neutrophil elastase inhibitor screening kit (Abcam, Cat #ab118971) according to the manufacturer's protocol. N-Acetly-eglin C peptide was purchased from Enzo Life Sciences (Cat #ALX-201-006-MC01) for use as a positive control.

ELISA. EGF positive secretion, negative secretion (noAB) and G3P2 positive secretion E. coli W3110 were grown overnight, and 100-fold dilutions were made in fresh LB medium. Once the cultures reached A₆₀₀=0.5, the medium was replaced with Ham's F-12 medium (Thermo Fisher Scientific, Cat #11765070) supplemented with 1 mg/ml BSA and 0.2% (v/v) arabinose and grown overnight for induction. Cell-free supernatants were collected, and the supernatants were directly used for detection of EGF with a Human EGF ELISA kit (Boster Bio, Cat #EK0325) according to the manufacturer's protocol. Standard EGF provided by the company was used as a positive control.

Mammalian cell culture, transfection and EGFR phosphorylation assay. CHO-K1 cells (ATCC, Cat #CCL-61) were grown in six-well plates at 1×10⁶ cells/well and transfected with 1.5 μg of pcDNA-EGFR-HAtag DNA using PEI (polyethyleneimine, Fisher Scientific, Cat #NC1014320) as previously described before in a ratio 3:1 [see, Longo, et al. Methods Enzymol 529, 227-240 (2013)]. After 18 hours, cells were washed three times with 2 ml Ham's F12 supplemented with 1 mg/ml BSA and incubated in this medium for 3 hours at 37° C. to serum starve. The different ligands were added in specific wells, a control of 100 ng/ml EGF purified as described by Qiu et al. (Biochemistry 48, 6624-6632 (2009)) for 5 min, the same supernatant samples used for EGF ELISA assay with estimated concentration of 100 ng/μl and a dilution 1:100 in Ham's media for 5 min at 37° C., as a G3P2 supernatant sample was used a “non-specific” peptide for negative control. Wells were washed with ice-cold phosphate buffered saline and then lysed for 30 min at 4° C. in 250 μl of RIPA buffer supplemented with 1 mM activated sodium orthovanadate, Pierce protease inhibitor minitablet (Thermo Fisher Scientific, Cat #A32955) and Benzonase nuclease (Sigma-Aldrich, Cat #E1014). Total protein concentration of clarified lysates was determined using the BCA (Bicinchoninic Acid) assay and lysates were normalized to the lowest total protein content using RIPA buffer. Normalized amounts of protein lysates were mixed with sample buffer and boiled, separated by SDS-PAGE 4-12%, and transferred onto a nitrocellulose membrane. The membrane was blocked with 3% low fat milk TBS, and proteins were detected by incubation with: rabbit anti EGF Receptor (D38B1) (Cell Signaling, Cat #4267), rabbit anti phospho-EGFR pTyr1068 antibody (Thermo Fisher Scientific, Cat #44-788G), rabbit anti-β-Actin (Cell Signaling Technology, Cat #4968) and the secondary antibody Goat anti-rabbit-680RD (Li-Cor, Cat #926-68071). Visualization was done as described above for the Western blotting procedure.

### Example 2. Development of Peptide Secretion System

A simplified scheme of MccV secretion via the MccV secretion apparatus is illustrated in FIG. 1A. The peptidase domain (PEP) of CvaB cleaves a 15-amino-acid signal peptide (generally SP, or CvaC15) of premature MccV during its export to extracellular space. The immunity protein cvi, which is located in the inner membrane and allows immunity for MccV, is omitted in FIG. 1A. A plasmid-based secretion system containing CvaA, CvaB and CvaC15 using two plasmids pBAD-18 Km and pACYC184 was constructed for the studies described herein (FIG. 1B). “Positive secretion” refers to bacteria expressing wild-type CvaA, wild-type CvaB, and a peptide of interest (POI) conjugated to CvaC15. The expression of POI is regulated by araBAD promoter, and CvaA/CvaB are constitutively expressed by a tetracycline-resistant gene promoter. “Negative secretion” refers to: (1) bacteria expressing only a CvaC15-conjugated POI and without expression of CvaA and CvaB (noAB); or (2) bacteria expressing a CvaC15-conjugated POI, wild-type CvaA, and mutant-type CvaB (C32S). The C32S mutation in CvaB is known to significantly decrease the secretion efficiency of the MccV system due to disruption of catalytic triad of PEP. It is generally accepted to use a strain which does not express one or more secretion apparatus components as a negative control in bacterial secretion studies. However, the present studies employ an additional negative control expressing a non-functional secretion apparatus protein. This additional control was used to identify potential false-positive secretion caused by lysis of bacteria due to irregular expression of membrane proteins.

To test whether the engineered secretion systems were functional, MccV and cvi were expressed and agar diffusion assays were conducted to detect MccV secretion. When plated on a lawn of sensitive E. coli, a zone of inhibition (ZOI) was observed around a microcin-secreting strain. Only E. coli W3110 encoding MccV, Cvi, and wild-type (WT) CvaAB (positive secretion) produced a visible ZOI against susceptible E. coli W3110 (FIG. 1C). Strains encoding MccV, Cvi, and empty pACYC184 (negative secretion) or the CvaB mutant (protease-deficient secretion) could not form a zone, indicating they could not secrete MccV. (FIG. 1C). It was confirmed that the expression of native TolC was enough for the secretion in E. coli W3110, so additional cloning of TolC into the engineered secretion system was not conducted.

Next, the secretion of peptides was confirmed biochemically using C-terminal V5-tagged MccV (MccV_V5). Supernatants and whole cell lysates were used to confirm the secretion of MccV_V5. In supernatant samples, MccC_V5 was only detected in positive secretion supernatant by Western blot (FIG. 2). Compared to whole cell lysate samples, DnaK proteins in supernatant samples were not detectable or significantly low regardless of positive or negative secretion, which implies that the impact of the constitutive expression of CvaA/CvaB is trivial in cell lysis. In whole cell lysate samples, MccC_V5 was detectable both in positive and two negative secretion systems. This indicates that the non-detection of MccC_V5 in negative secretion supernatants was not a false negative caused by improper expression of the substrate. In cell lysates, the size of MccV_V5 in positive secretion samples was smaller (about 1 kDa to 2 kDa) compared to negative secretions samples, comparable to the size of MccV_V5 without CvaC15. The shifting pattern is consistent with previous studies, indicating correct processing (cleavage) of pre-mature MccV. Although the secretion process is suggested as one-step, meaning cargo peptides will be directly released from cytoplasm to extracellular space, cargo peptides might stay for a while in cytoplasm-periplasmic space where the CvaB is located during the cleavage of the CvaC15. Since secretion or cellular expression from the two “negative secretion” systems (no AB and C32S, FIG. 1B) appeared to be identical, the two systems were used interchangeable in further analyses.

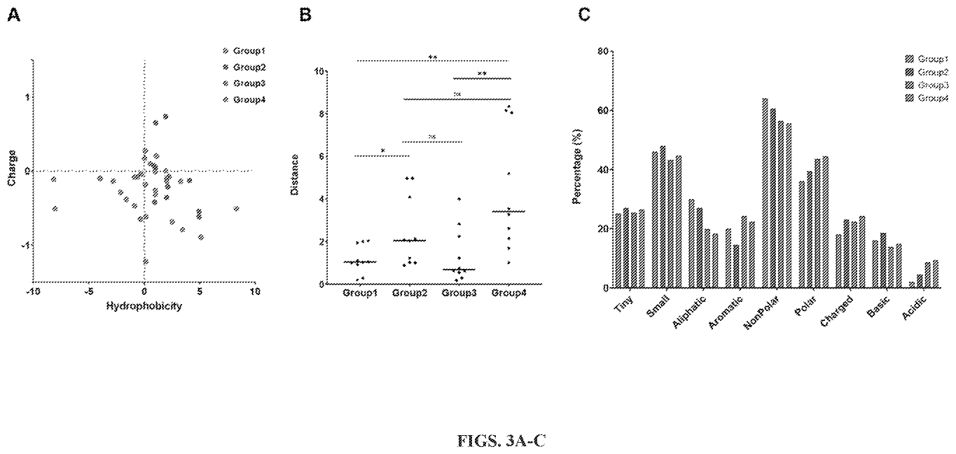

To streamline the process for detection of peptide expression, centrifuged culture samples and boiled culture samples were assessed directly by dot blot assay. The dot blot assay results were consistent with Western blot results (FIG. 2B). Interestingly, the cellular expression level for the “negative secretion” samples were lower than that of “positive secretion” samples. This is consistent with previous studies suggesting that cargo peptide is more likely to be degraded due to improper translocation in the absence of CvaA and CvaB. The secretion of MccV_V5 was also confirmed via agar diffusion assay. Only “positive secretion” E. coli made a visible zone of inhibition as shown in FIG. 3C.

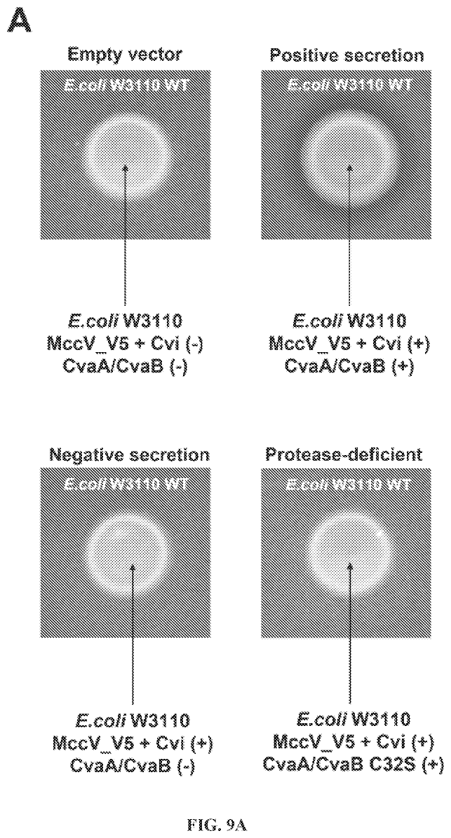

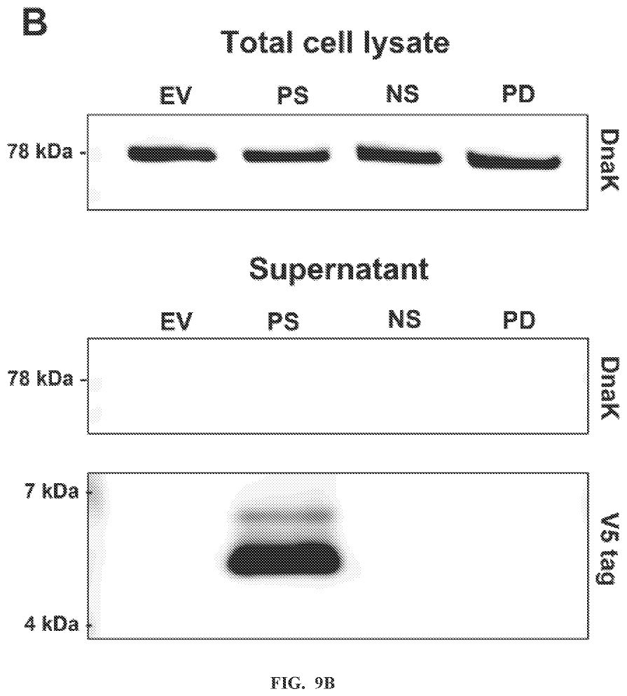

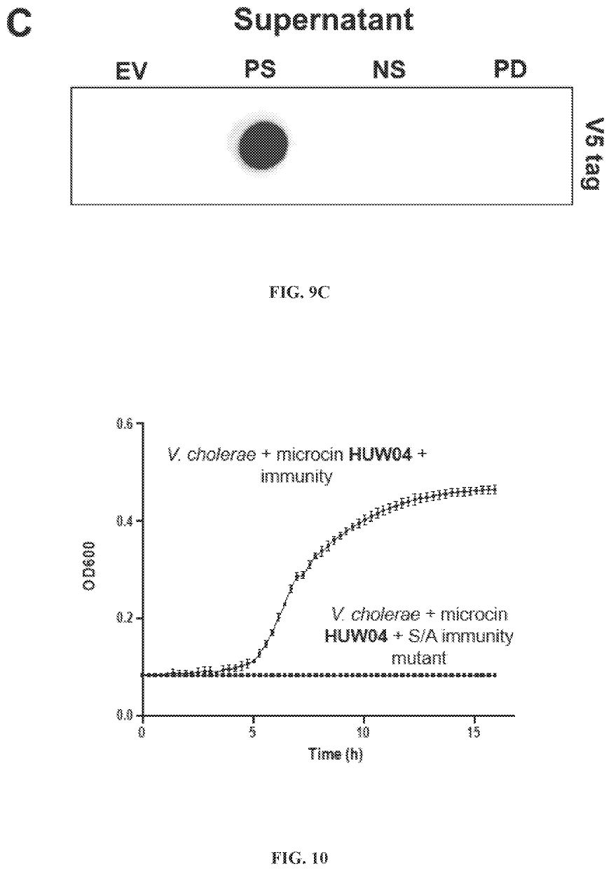

In another experiment, it was shown that MccV_V5 fusion retained its inhibitory activity (FIG. 9A). Only secreting E. coli W3110 encoding both MccV_V5, Cvi, and WT CvaAB could inhibit susceptible E. coli W3110 (FIG. 9A). This result indicates that MccV_V5 is secreted and shows the same dependency on CvaAB as the native MccV peptide.

Expression of these strains was induced for two hours in rich medium, the cell supernatant was collected, and the presence of MccV_V5 was investigated. In supernatant from our positive secretion strain (PS), a dominant band migrating near 5-6 kDa was detected, which is consistent with previous observations of MccV (FIG. 9B). A weaker band migrating at a slightly higher molecular weight that likely represents unprocessed precursor MccV_V5 was also observed. This suggests a small amount of peptide is able to escape without N-terminal processing. MccV_V5 was only detected from bacteria that encoded WT CvaAB (FIG. 9B). This result is consistent with the ZOI results in Fig. XXA and further supports the dependence of peptide secretion on CvaAB. Cytoplasmic protein, DnaK was not observed in any of the supernatant samples, but was readily observed in total cell lysate, indicating that bacteria were not lysing during secretion of MccV_V5. To simplify the process for detecting secreted peptides, we tested dot blot detection of MccV_V5 from culture supernatants (FIG. 9C). The result was consistent with the western blot analysis (FIG. 9B). These results support the use of a two-plasmid platform and dot blotting to detect secreted peptides.

### Example 3. Secretion Studies Using Random Synthetic Peptides

To examine the capacity of the MccV system to secrete diverse peptides, a total of forty random peptide sequences that are C-terminal V5 tag conjugated was generated (Table 11). All of the peptides contained two glycine residues between the synthetic POI sequence and the V5 tag to allow for the flexibility of the epitope tag. The peptides consisted of four groups, and each group included ten different peptides having the same length but different sequences (Table 14). Groups 1, 2, 3, and 4 encode 10, 20, 50, and 100 random amino acids, respectively. Group 1, group 2, group 3 and group 4 contained 26-mer, 36-mer, 66-mer and 116-mer peptides, respectively, including the two glycine residues and the C-terminal V5 tag. Different sizes of peptides were studied to provide a better insight into how the MccV system secretes cargo peptides that are smaller or larger than its original substrate, MccV (88-mer, without CvaC15).

TABLE 14
Properties of random synthetic peptides.

| | | | SEQ | | | |
|---|---|---|---|---|---|---|
| | | | ID | MW | Hydro- | |
| ID | Group | Sequence | NO: | (g/mol) | phobicity | Charge |
| G1P1 | 1 | GIGWMLSRARGGGKPIPNPLLGLDS | 574 | 2664.15 | 2.00 | −0.09 |
| | | T | | | | |
| G1P2 | 1 | IVHRYPRICYGGGKPIPNPLLGLDST | 575 | 2837.37 | 2.03 | −0.14 |
| G1P3 | 1 | SHSMVSVLVRGGGKPIPNPLLGLDS | 576 | 2632.10 | 1.09 | 0.20 |
| | | T | | | | |
| G1P4 | 1 | GIYGHIVVYWGGGKPIPNPLLGLDS | 577 | 2724.18 | 0.09 | 0.27 |
| | | T | | | | |
| G1P5 | 1 | SGLRPWMASVGGGKPIPNPLLGLDS | 578 | 2621.08 | 1.00 | −0.01 |
| | | T | | | | |
| G1P6 | 1 | LSMSICMRPKGGGKPIPNPLLGLDST | 579 | 2683.28 | 1.94 | 0.00 |
| G1P7 | 1 | VNDRLKLQWVGGGKPIPNPLLGLD | 580 | 2788.27 | 1.00 | −0.26 |
| | | ST | | | | |
| G1P8 | 1 | PGLIDVSYWHGGGKPIPNPLLGLDS | 581 | 2704.10 | −0.91 | −0.08 |
| | | T | | | | |
| G1P9 | 1 | SHSVAPWSLQGGGKPIPNPLLGLDS | 582 | 2628.99 | 0.09 | −0.18 |
| | | T | | | | |
| G1P10 | 1 | RYFTLNNFGWGGGKPIPNPLLGLDS | 583 | 2835.24 | 1.00 | −0.32 |
| | | T | | | | |
| G2P1 | 2 | GSVTRFISFWHMMLLCGMLVGGGK | 584 | 3846.70 | 1.03 | 0.65 |
| | | PIPNPLLGLDST | | | | |
| G2P2 | 2 | SVIRINCLVLVGRLVLGTQVGGGKP | 585 | 3670.46 | 1.94 | 0.74 |
| | | IPNPLLGLDST | | | | |
| G2P3 | 2 | GLWRAFMPWWSFFGVVRDSGGGG | 586 | 3919.56 | 1.00 | 0.05 |
| | | KPIPNPLLGLDST | | | | |
| G2P4 | 2 | ALLRRDVSFRFWHRSVLYVRGGGK | 587 | 4094.82 | 4.09 | −0.13 |
| | | PIPNPLLGLDST | | | | |
| G2P5 | 2 | NCDRWRGRWVRFILWFGRGKGGG | 588 | 4126.85 | 4.94 | −0.54 |
| | | KPIPNPLLGLDST | | | | |
| G2P6 | 2 | HNPRRHWMGLITLKLMSCDLGGGK | 589 | 3939.72 | 2.12 | −0.21 |
| | | PIPNPLLGLDST | | | | |
| G2P7 | 2 | SVCPAFRVDTFRSTGDKYSNGGGKP | 590 | 3768.26 | 0.94 | −0.42 |
| | | IPNPLLGLDST | | | | |
| G2P8 | 2 | RQRDRCNPWMPIRAKRSLLVGGGK | 591 | 4013.79 | 4.94 | −0.62 |
| | | PIPNPLLGLDST | | | | |
| G2P9 | 2 | GNARCSMCEIRPLMVWTVSTGGGK | 592 | 3772.48 | 0.88 | 0.07 |
| | | PIPNPLLGLDST | | | | |
| G2P10 | 2 | GVSYNTMVQRRGGDPARALMGGG | 593 | 3697.29 | 2.00 | −0.36 |
| | | KPIPNPLLGLDST | | | | |
| G3P1 | 3 | LSNGQCNMHCCPCLEYQDYHKHYS | 594 | 7553.66 | 0.14 | −0.62 |
| | | NTESFKQLVWMTHICDNYALSHRA | | | | |
| | | KWGGGKPIPNPLLGLDST | | | | |
| G3P2 | 3 | HGVQGINIEKQPKRNNPENEQTRM | 595 | 7583.53 | 0.13 | −1.22 |
| | | KMRQERDWSCFAMFHAITRDDSYE | | | | |
| | | QNGGGKPIPNPLLGLDST | | | | |
| G3P3 | 3 | ETCHWMELHIPLFETDSFKPYDPKS | 596 | 7488.72 | −2.82 | −0.14 |
| | | LDSGHCLYYGFFFKYIGGLHAMCM | | | | |
| | | YGGGKPIPNPLLGLDST | | | | |
| G3P4 | 3 | VQMIWGFCSGWPMITWYAMMFAH | 597 | 7633.00 | 0.03 | 0.17 |
| | | IQWAFWNTKISSRGWEFMASAWPE | | | | |
| | | YFVGGGKPIPNPLLGLDST | | | | |
| G3P5 | 3 | IMECSTLICTTLDCFICVQGQWRYC | 598 | 7466.90 | 0.53 | 0.10 |
| | | MWNQCWCVCMNWTKTNCAQYSV | | | | |
| | | AKHGGGKPIPNPLLGLDST | | | | |
| G3P6 | 3 | IICWWPHPQCCWNFEYCFRKNYLT | 599 | 7710.00 | −0.28 | −0.04 |
| | | CFQCSEQYSTTFAPVNAFPTIWQIIY | | | | |
| | | GGGKPIPNPLLGLDST | | | | |
| G3P7 | 3 | GSVIVLDWVQGTNLVHKQHNTINR | 600 | 7527.57 | −0.36 | −0.65 |
| | | RHHHSHQEMYPWPTVFEMHGNRII | | | | |
| | | EEGGGKPIPNPLLGLDST | | | | |
| G3P8 | 3 | HVPKWWYKGFDWTTQVWPYAAM | 601 | 7529.77 | −0.61 | −0.08 |
| | | LGFINAHHLDTVMIKIHLGAFHNCD | | | | |
| | | WVEGGGKPIPNPLLGLDST | | | | |
| G3P9 | 3 | VTTFKLWAKALVAFMYDADHHVN | 602 | 7543.86 | 2.23 | −0.07 |
| | | DFLPTLYRVYTTMNIWHFKHKCPC | | | | |
| | | TSYGGGKPIPNPLLGLDST | | | | |
| G3P10 | 3 | DDDDELLRLCDNITFFMMCIHEFTM | 603 | 7625.89 | −4.00 | −0.10 |
| | | KPWFKTIWFLMCWNAFLNQGSNST | | | | |
| | | HGGGKPIPNPLLGLDST | | | | |
| G4P1 | 4 | YMCWHQAINVMEPCAQFYQDIVLS | 604 | 13715.07 | −2.13 | −0.29 |
| | | SRVQWQDMDMSMPRLYKMQYVA | | | | |
| | | KSHFSIMYFIHREDIQSSGCCDCNCN | | | | |
| | | PVRKIFCTRYVEMVDCGYWRHFWL | | | | |
| | | PPEEGGGKPIPNPLLGLDST | | | | |
| G4P2 | 4 | FIIYIPKVYDWYAASMGCTYPDGLG | 605 | 13608.64 | −0.88 | −0.47 |
| | | FRMVSMRVIWYAWYVYSCYTAVE | | | | |
| | | TNKFGDCGCGNYNHPDQEQIRYKT | | | | |
| | | SEQESTKMFFRWAPEWQNNHRHHL | | | | |
| | | MVEMGGGKPIPNPLLGLDST | | | | |
| G4P3 | 4 | HSYCEWLMAKILSMGEQWWHKYY | 606 | 13688.57 | 3.45 | −0.79 |
| | | FGLRHQFNVSKGYHFNTSFDFHRCG | | | | |
| | | EPKNNYYARTHCEKEYSNDDVHPR | | | | |
| | | QNSMGRWGAEWPALIFKPLFGINK | | | | |
| | | WGNFGGGKPIPNPLLGLDST | | | | |
| G4P4 | 4 | SGIYCQITRWVHPFSESTQNMDNM | 607 | 13624.55 | 2.52 | −0.69 |
| | | ANTKRKPQWHYPRRHQHKEAQFVF | | | | |
| | | FGIARGWPFQFFEQGWVTIDVHEEH | | | | |
| | | LWIFCFGERNMEHGNVERAPTISSRI | | | | |
| | | KGGGKPIPNPLLGLDST | | | | |
| G4P5 | 4 | KHWKQRHVYYRQAVYQQQKCQM | 608 | 13393.27 | 5.11 | −0.89 |
| | | YNVPYSPASGCFNCQPNCHRKDFD | | | | |
| | | WRDHTGYCFKMLQFHNFITPKRCT | | | | |
| | | ESALFAGQECRPDFAQESGAQKDFS | | | | |
| | | GMHPGGGGKPIPNPLLGLDST | | | | |
| G4P6 | 4 | FNPESSHQPSTIPKSHFRIICHFVRDW | 609 | 12962.73 | −1.64 | −0.38 |
| | | HFPCGSWTFSVVDIYFCEMYTTLGN | | | | |
| | | PHGFVICCTYGSQYSGDNRCADKLE | | | | |
| | | RHPAMMENTYGWHGHTSAGLAQP | | | | |
| | | GGGKPIPNPLLGLDST | | | | |
| G4P7 | 4 | DKMEPQWNHSPRCFASLCCGGSHT | 610 | 12878.07 | 3.27 | −0.14 |
| | | MMAWNHVISWKGRDLVIGVNRHC | | | | |
| | | ATPPHSQFWHNAWWQGFIKHVIET | | | | |
| | | PRLGNMAKNMQCFAACALAVAFP | | | | |
| | | VDISQLGGGKPIPNPLLGLDST | | | | |
| G4P8 | 4 | RGKWMMCNTHMAHWNTYFWDGS | 611 | 13910.21 | 8.32 | −0.51 |
| | | AVHLTDDFYRNGPAKSYNLFMVQN | | | | |
| | | HKESRHNYKVCFFCYLITTYITIAKH | | | | |
| | | KRMNENYWWMAQVYLKFVRWHA | | | | |
| | | RNCCYAGGGKPIPNPLLGLDST | | | | |
| G4P9 | 4 | VMQGDTWWECEPSEAEIQMLYWP | 612 | 13255.01 | −8.03 | −0.51 |
| | | WGSQKDPIDWAYLCDTWKYTGDL | | | | |
| | | CSGGPEQPDEHRIHDAIGRAFYRPCP | | | | |
| | | SLNMYYLSQRWAIFDTHNSLAAGS | | | | |
| | | YCFMGGGKPIPNPLLGLDST | | | | |
| G4P10 | 4 | TGRQVTEVTVWHALTTICGISELEF | 613 | 13468.68 | −8.15 | −0.11 |
| | | TYGACPMWENMELEKFSGNVCYEL | | | | |
| | | QDHCFCDWWQYTERCLENLPMIEL | | | | |
| | | PIQWKPFTLHEWWIIGRCPLTIMNS | | | | |
| | | WAGGGKPIPNPLLGLDST | | | | |

Theoretical charges and hydrophobicity were calculated and plotted for each group (FIG. 3A) to analyze how the generated peptides represent unbiased-random sequence space. To inspect the level of distribution per group, the distance between (a) each plotted peptide and (b) the point where both charge and hydrophobicity are zero, was measured (FIG. 3B). This analysis showed that group 1 and group 3 are more condensed around the zero point compared to group 2 and group 4, implying that the peptides in each group do not have a particular, distinct distribution of physicochemical properties. In addition, amino acid composition per group was analyzed with classification of amino acids into conventional Tiny, Small, Aliphatic, Aromatic, Non-Polar, Polar, Charged, Basic, and Acidic classes. Each composition was similar to other groups in general, which indicated that none of groups is significantly biased in amino acid composition.

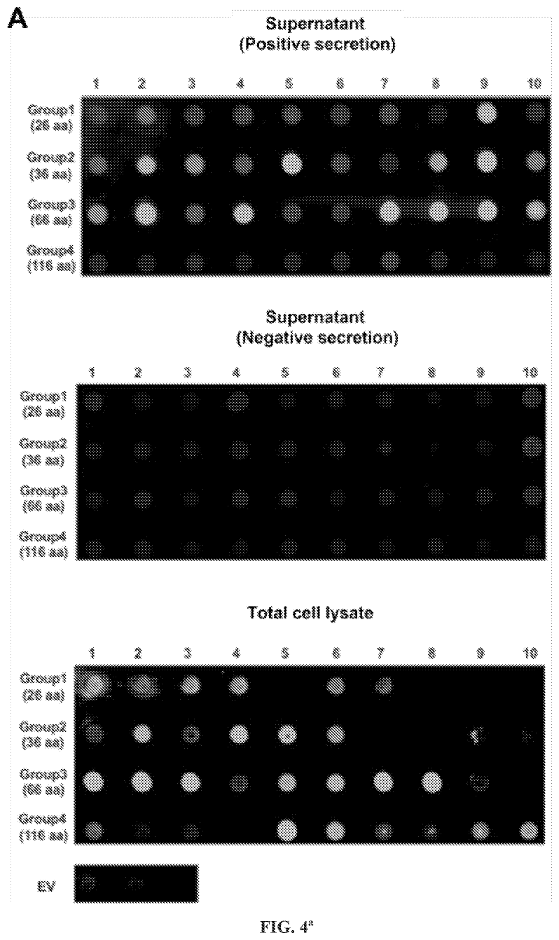

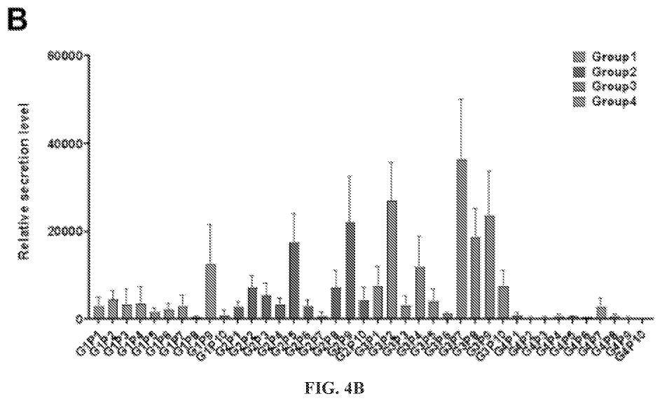

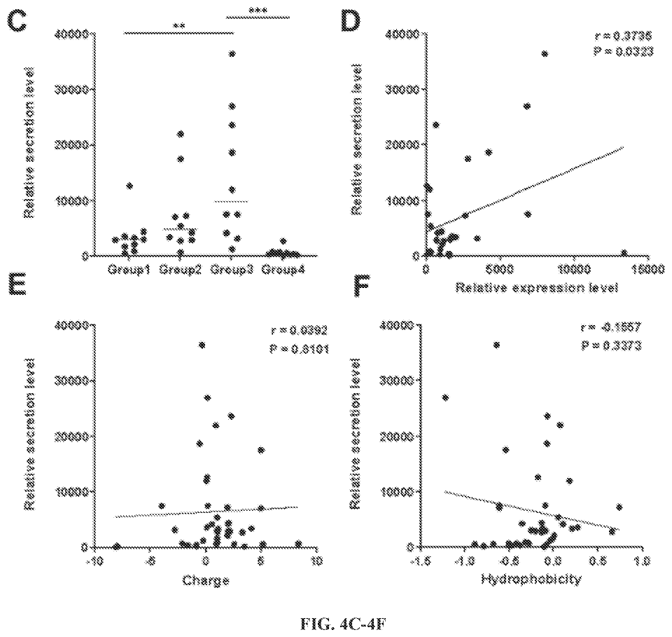

Secretion levels of random synthetic peptides. The constructed random peptides were expressed in E. coli W3110 with (positive secretion) or without (negative secretion) CvaAB to analyze their secretion levels. Since it was confirmed that dot blotting followed by Infrared dye secondary antibody incubation was an efficient way to detect peptides in supernatant samples (FIG. 2B), dot blot assays against V5 tag were performed and the infrared signal intensity was taken as a representation of the relative amounts of peptides in supernatant samples (FIG. 4A). To calculate secretion levels of the peptides, both positive and negative secretion signal intensities were first divided by A₆₀₀ values measured upon collection of the supernatant samples for the purpose of normalization (A₆₀₀=1.0). Then, the signal intensity of negative secretion was subtracted. The obtained signal intensity, or secretion level, of each peptide was graphed in FIG. 4B and the distribution of secretion level per group showed in FIG. 4C. In general, secretion levels gradually increased from group 1 to group 3, but group 4 had a significantly lower secretion level compared to all other groups (at least P<0.05). This data indicated that a cargo peptide around 13 kDa may not be a favorable substrate for MccV-based systems.

Next, other properties of a peptide that may be associated with its secretion were studied. To have a better insight, a regression assay was used for assessment of whether expression level (or intracellular amount), charge, or hydrophobicity of peptide is related to secretion level. Signal intensities from dot blot assays of whole cell lysates were used to calculated cellular expression levels (FIG. 4A), which were normalized by A₆₀₀ values prior to subtraction of the signal intensity of cell lysate that neither expressed a cargo peptide nor secretion machinery proteins (empty vector, EV). The negative secretion (no AB) control was used in order to better understand the absolute relationship between peptide's expression level with secretion level. This is because the expression of the machinery proteins may affect the amount of peptide in cell as shown in FIG. 2B, which might provide ambiguity in measuring absolute expression of the target peptide. The results showed that charge, hydrophobicity, and expression level did not have a strong relationship with secretion level (FIG. 4D-F). There was a slight positive relationship between expression level and secretion level, which is not surprising when considering that a more highly expressed substrate has a greater chance of being released. Seven peptides in which calculated expression levels were below zero (G1P5, G1P10, G2P7, G2P8, G2P9, G2P10, and G4P3) were not included in the regression assay.

Absolute secretion levels of secreted peptides. The absolute secretion levels of selected peptides was measured via dot blot (FIG. 5A). G1P9, G2P9, G3P2, and G4P7 were quantified as these peptides showed the highest secretion compared to those of other peptides in their own group. The MccV_V5 peptide's secretion level was also measured since it would represent the secretion levels of MccV, the natural substrate peptide of the MccV system. For absolute quantification, two synthesized peptides, NPS_V5 (SFRNGVGSGAKKTSFRRAKQGGKPIPNPLLGLDST; SEQ ID NO:572) and ECP_V5 (YRWRCKNQGGGKPIPNPLLGLDST; SEQ ID NO: 573) were used. In order to adjust signal intensities within the range generated by the standard peptides, each supernatant sample was diluted by 25-fold. The secretion level of each peptide was calculated as described above and converted to μM based on a linear standard curve generated by both NPS_V5 and ECP_V5 (FIG. 5B and FIG. 5C). The absolute secretion levels of peptides were consistent with the previous results (FIG. 4), which showed levels in the order of G3P2, G2P9, G1P9 and G4P7. The higher secretion level of G3P2 compared to the secretion level of MccV_V5 shows that a heterologous cargo peptide can be secreted by the MccV system at comparable level to natural cargo.

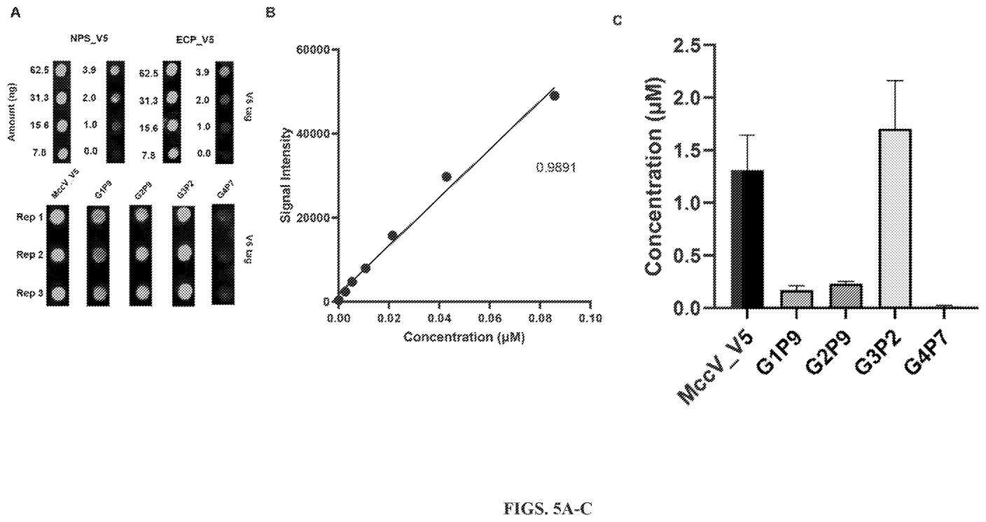

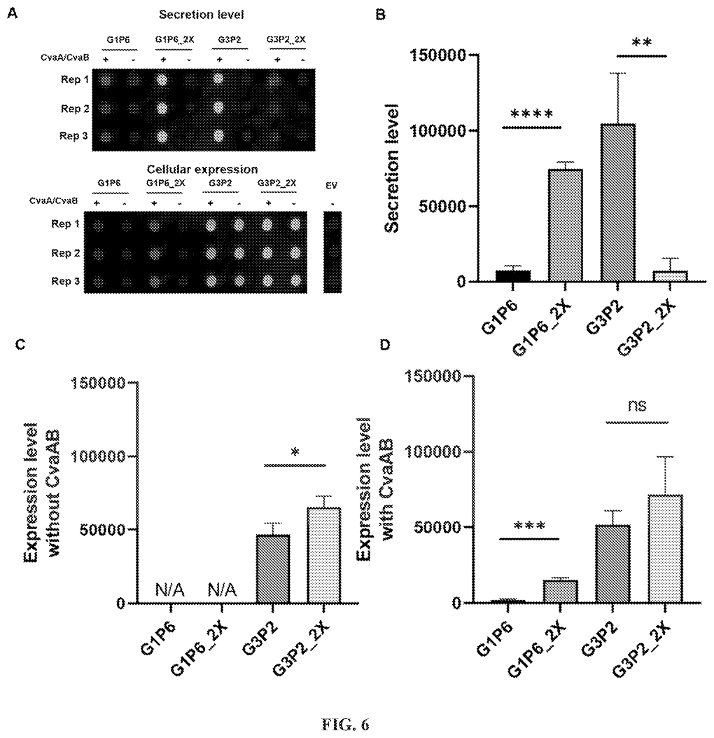

Dependency of secretion levels on the size of cargo peptide. The results in FIG. 4 imply that the size of peptide (rather that charge, hydrophobicity, or expression level), is a main factor that affects secretion level. Two peptides GIP6 and G3P2, which represents a small and large peptide, respectively, were selected to test this hypothesis. The peptides were used to generated new constructs, G1P6_2X and G3P2_2X, whose sequences were lengthened by adding a direct repetition of the peptide sequences. Due to repetition, 2X peptides have similar biochemical properties to the original peptides, but their sizes are nearly 2-fold larger than the original peptides, and are identical to the length of group 2 and group 4 peptides, respectively. Secretion levels and expression levels were measured by performing dot blot assays (FIG. 6A). In the case of GIP6 and G1P6_2X, the secretion level of the larger peptide was significantly higher, but expression levels of both peptides, with the absence of secretion machinery proteins, was not detectable. Interestingly, in the presence of CvaA/CvaB, the cellular expression of GIP6_2X was significantly higher than G1P6. This indicated that G1P6_2X was more affected by CvaA/CvaB proteins, compared to G1P6, and the impact increased G1P6_2X's intracellular amount, which is more likely to allow for efficient secretion. Otherwise, it could be simply be interpreted to mean that lengthening G1P6 will lead to more secretion. In FIG. 6, G3P2_2X showed significantly less secretion than that of G3P2, although G3P2_2X has higher intracellular expression regardless of the existence of secretion apparatus proteins. It was hypothesized that G3P2_2X could not efficiently pass through secretion machinery proteins due to its size. Altogether, these experiments show the size preference of the MccV system when it secretes a heterologous cargo peptide.

### Example 4. Secretion of Bioactive Heterologous Peptides by the MccV System

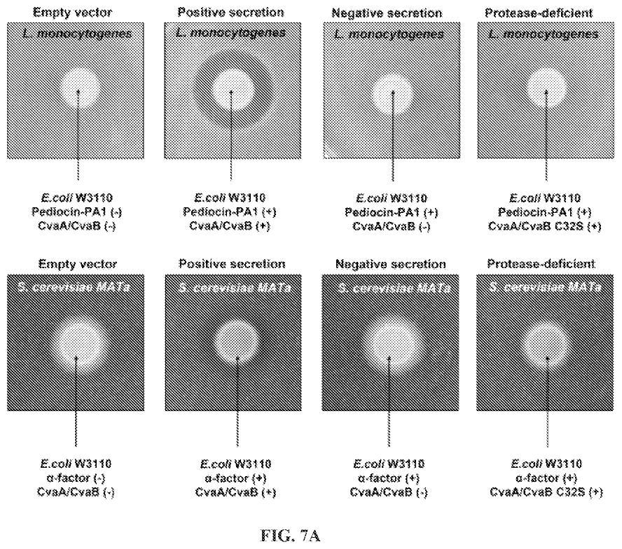

After characterizing properties of peptides that are highly compatible with the MccV system, the ability to secrete heterologous peptides other than bacteriocins was confirmed. Four peptides, Pediocin PA-1, α-factor, Eglin C, EGF (Epidermal Growth Factor), were selected for the study. The functions and properties of the peptides are listed in Table 15. Each peptide was expressed with the N-terminal SP from MccV to direct their export. First, the secretion of Pediocin PA-1 and α-factor was confirmed by agar diffusion assay. ZOI assays (as performed for MccV (FIG. 1C)) were conducted, secreting pediocin from E. coli W3110 spotted on a lawn of L. monocytogenes. When E. coli expressed both pediocin and CvaAB, it inhibited L. monocytogenes growth and produced a ZOI (FIG. 7A). No zone was observed with a negative secretion strain (no CvaAB) or protease-deficient secretion strain (CvaB C32S mutant) confirming CvaAB-dependent secretion and action of pediocin PA-1. This indicates the MccV secretion system can be used to produce and deliver heterologous peptide antibiotics.

TABLE 15
Selected bioactive peptides

| | | | MW | Hydro- | |
|---|---|---|---|---|---|
| Name | Function | Origin | (g/mol) | phobicity | Charge |
| Pediocin | Anti-bacterial peptide, | Pediococcus | 4628.19 | −0.49 | 3.02 |
| PA-1 | strongly inhibits L. | acidilactici | | | |
| | monocytogenes | | | | |
| α-factor | Peptide pheromone, arrests | Saccharomyces | 1683.99 | −0.87 | 1.09 |
| | cell cycle | cerevisiae | | | |
| Eglin C | Protease inhibitor, inhibits | Hirudo | 8091.05 | −0.41 | −1.72 |
| | neutrophil elastase | medicinalis | | | |
| EGF | Epidermal Growth Factor, | Homo sapiens | 6353.21 | −0.38 | −4.19 |
| | activates EGFR signaling | | | | |

The same assay was used for α-factor production. α-factor is a pheromone released by Saccharomyces cerevisiae mating-type alpha cells that activates its G-protein-coupled receptor (GPCR) Ste2p causing cell-cycle arrest in susceptible S. cerevisiae strains (MATa) (23). FIG. 7A shows that, when E. coli W3110 expresses both signal peptide-fused α-factor and CvaAB, it can inhibit the growth of susceptible MATa S. cerevisiae. This indicates the MccV system can be used to secrete peptides active against GPCRs and impact evolutionarily distant organisms.

For Eglin C, a neutrophil elastase (NE) activity assay was carried out to confirm the secretion of Eglin C via the MccV system. As shown in FIG. 7B, NE activity with Eglin C positive secretion was comparable to positive control, which is NE with purified Eglin C (1.5 μM) while NE activity with Eglin C negative secretion showed a similar level with the sample having only NE. This indicated that Eglin C was successfully secreted via the MccV system and retained its activity.

Prior to testing the secreted EGF's activity via cell-based EGFR (Epidermal growth factor receptor) phosphorylation assay, the relative amount of EGF in supernatant samples was measured. To treat cell-free supernatant samples directly to mammalian cells in the activity assay, samples were expressed in Ham's F-12 media.

A colorimetric ELISA (enzyme-linked immunosorbent assay) against 1:100 diluted EGF positive secretion, EGF negative secretion and G3P2 positive secretion with a standard EGF (1 ng/ml) control, was performed. G3P2, one of the random synthetic peptides generated for this study, was expected to be a nonspecific peptide control for EGFR. However, it was tested so as to determine whether it shares similar epitopes with EGF that might compromise its usage as the control for EGFR phosphorylation assay. As expected, only EGF positive secretion sample showed a comparable ELISA signal to that of control, even though the sample was diluted by a factor of 100. Through ELISA, it was concluded that the supernatant sample contains at least 100 ng/ml of EGF which is the required concentration for EGFR phosphorylation assay in a previous study. The result of the EGFR phosphorylation assay shown in FIG. 7D. It was confirmed that the EGF positive secretion supernatant increased immunoblot signal of the phosphorylated Y1068 residue of EGFR, which suggested the secreted EGF activated its cognitive receptor.

### Example 5. Comparability of MccV System with Other Gram-Negative Bacteria

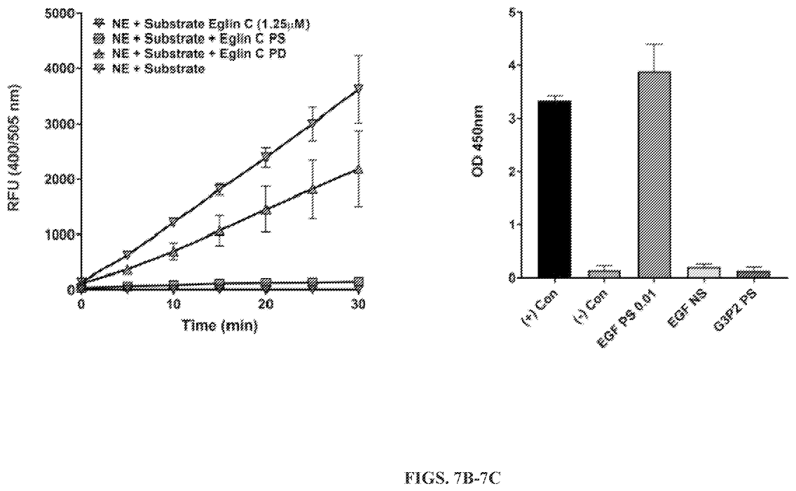

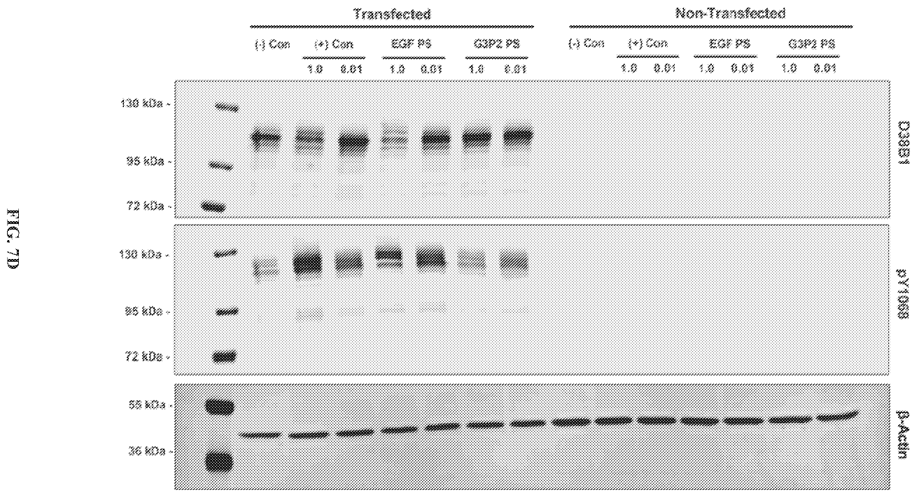

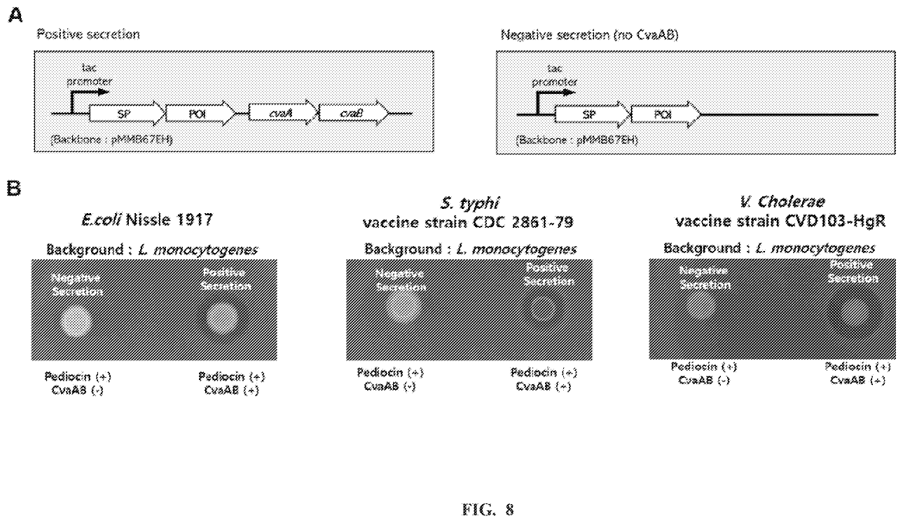

The studies above demonstrate the MccV system's capacity for secretion of heterologous peptides in E. coli W3110. However, the comparability of the MccV system with other gram-negative bacteria is not well-known. Another MccV-based secretion system was constructed using a broad-host-range plasmid, pMMB67EH by cloning cvaA and cvaB into the plasmid (FIG. 8A). In this instance “positive secretion” refers to a plasmid for expression of CvaC15-POI, CvaA and CvaB. “Negative secretion” refers to the expression of CvaC15-POI without CvaA/CvaB. E. coli Nissle 1917 (EcN), and vaccine strains of Salmonella enterica and Vibrio cholera were manipulated to secrete Pediocin PA-1 through the systems. It was confirmed that only bacteria strains having the positive secretion system were able to make zones of inhibition against L. monocytogenes that represents successful secretion of Pediocin PA-1 (FIG. 8B).

Further evidence for MccV system's capacity for secretion of heterologous peptides was shown by expressing and secreting all of the peptides listed in Table 1 (i.e., SEQ ID NOs: 683-695), all of the microcins listed in Table 2 (i.e., SEQ ID NOs: 696-729, and all of the affibodies listed in Table 4 (i.e., SEQ ID NOs: 730-735) using the MccV system described herein.

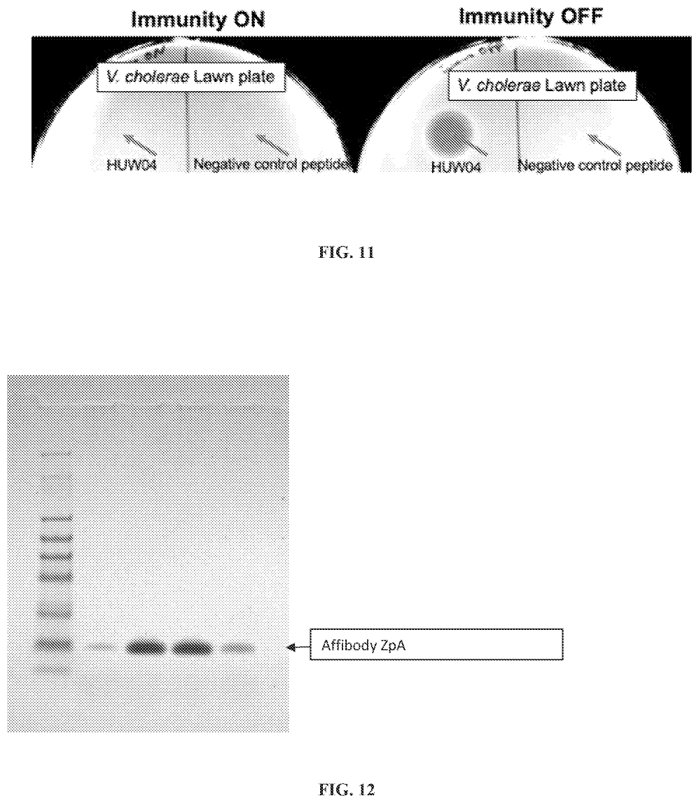

Representative results from these studies are described below. FIG. 10 shows results of experiments conducted to express microcin HUW04 in E. coli using the MccV system, as described above. In these experiments, bacteria secrete microcin HUW04 in the presence of its immunity protein or a defective immunity protein (S/A). Bacterial growth was measured over time (OD₆₀₀). As shown in FIG. 10, in the presence of defective immunity protein, microcin HUW04 kills the bacteria, observed by no change in growth. In the presence of the immunity protein protecting against HUW04 activity, the bacteria grows exponentially. FIG. 11 shows the results of experiments where purified microcin HUW04 or a control peptide were spotted on bacteria that express (Immunity ON) or do not express (Immunity OFF) the HUW04 immunity protein. As shown in FIG. 11, HUW04 kills bacteria when immunity protein is off, as evident by the zone of clearance. The control peptide has no effect in either case.

As set forth above, all of the affibodies listed in Table 4 (i.e., SEQ ID NOs: 730-735) were expressed and secreted in E. coli, using the MccV system described herein. These affibodies were purified from the bacteria supernatant, as described above. Gel Coomasie staining was used to confirm purification of each affibody. FIG. 12 is an exemplary Coomassie stained gel for affibody ZpA, purified from the bacteria supernatant after secretion.

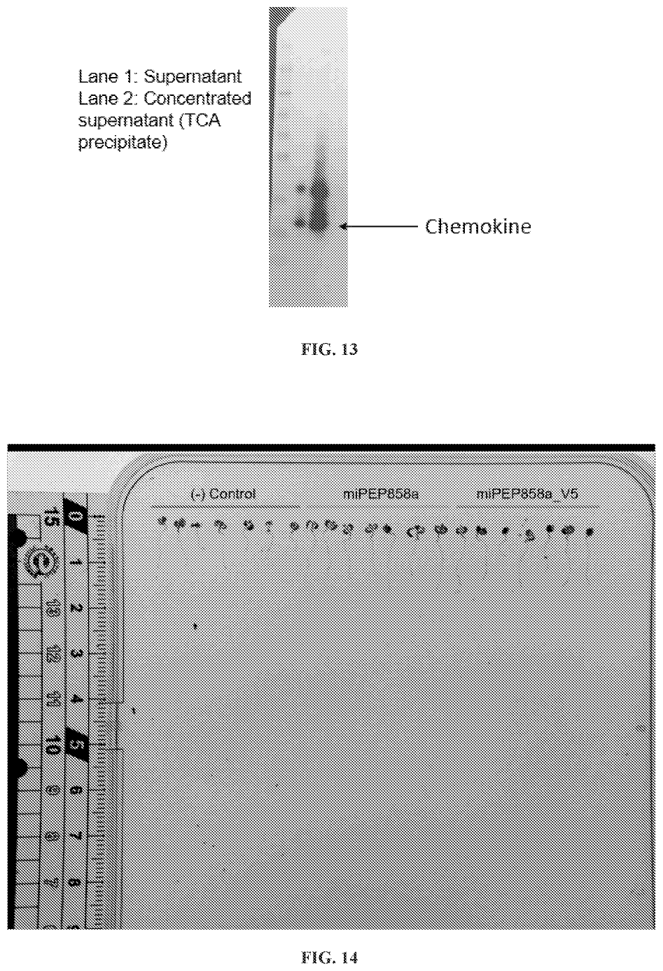

As set forth above, all of the peptides listed in Table 1 (i.e., SEQ ID NOs: 683-695) were expressed and secreted in E. coli, using the MccV system described herein. Western blot analysis was used to confirm expression of the peptides in bacteria supernatant. mCCL21, an exemplary chemokine, was secreted from bacteria and detected with an anti-CCL21a primary antibody, and visualized with an HRP-conjugated secondary antibody (FIG. 13).

### Example 6. Expression of Agricultural Peptides

The studies above demonstrate the MccV system's capacity for secretion of heterologous peptides in E. coli W3110. This includes peptides relevant to agriculture. As shown in FIG. 14, supernatant from E. coli secreting root growth stimulating peptide miPEP858a or a variant miPEP858a-V5 stimulated root growth of Arabidopsis compared to supernatant from control E. coli cultures.

Therefore, the MccV system described herein can be used to express and secrete a variety of heterologous peptides, from different species (e.g., from non-bacterial species) in bacteria (e.g., E. coli), making the MccV system a versatile system for producing heterologous peptides. Further, the peptides are properly folded and functional, allowing for production of many types of heterologous peptides, including therapeutic peptides and agricultural peptides.

### Example 7. In Vivo Studies

Animal models of gastrointestinal (gut) disease or inflammation are available to assess the effects of therapeutic agents on inflammation. See, for example, Antoniou et al. “The TNBS-induced colitis animal model: An overview,” Ann Med. Surg. (Lond) 11:9-15 (2016); Silva et al. “Chronic Experimental Model of TNBS-Induced Colitis to Study Inflammatory Bowel Disease,” Int. J. Mol. Sci. 23, 4739 https://doi.org/10.3390/ijms23094739; Jiminez et al. “Animal models to study acute and chronic intestinal inflammation in mammals,” Gut Pathogens 7: Article No. 29 (2015); Eichele and Kharbanda, “Dextran sodium sulfate colitis murine model: An indispensable tool for advancing our understanding of inflammatory bowel diseases pathogenesis,” World J. Gastroenterol. 23 (33): 6016-6029 (2017)); or Wang et al. “Oxazolone-induced murine model of ulcerative colitis,” 5 (4): 165-8 (2004), all of which are incorporated herein by this reference.

To treat disease or reduce inflammation, mice will be orally gavaged with bacteria secreting the desired peptide (e.g., a microcin, hormone, affibody, etc. including the microcins, hormones, affibodies, etc. described herein). Gavage volumes will vary and can be up to 10 ml/kg, and may be administered 1-3 times per day. Bacterial doses will range from 10e3-10e11 CFUs per gavage. Treatments will last from about 1 to about 10 days, depending on the disease. Treatment outcomes, for example, a decrease in gut inflammation, will be monitored depending on the disease. For example, changes in microbiome composition, markers of inflammation in the feces or blood (e.g., tumor necrosis factor (TNF), C-reactive protein, and/or calprotectin, to name a few), presence of specific metabolites in the feces or blood (e.g., short chain fatty acids or lipopolysaccharides), and/or histopathological changes in the gut, including structure and health of the luminal epithelium, can be monitored. It is understood that any animal model described herein or available to those of skill in the art can be used to determine the effectiveness of any gram-negative bacteria expressing a therapeutic peptide described herein, on gut inflammation or any disease affected by the gut microbiome.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A gram-negative bacterial cell comprising:
a first nucleic acid encoding a secretion signal sequence fused to a heterologous peptide, a second nucleic acid encoding a C39 peptidase-containing ATP-binding cassette transporter (PCAT), and a third nucleic acid encoding a membrane fusion protein; and
an inner membrane surrounding cytosol, an outer membrane surrounding the inner membrane, and periplasmic space between the inner membrane and the outer membrane;
wherein the gram-negative bacterial cell secretes the heterologous peptide from the bacterial cytosol to an external environment outside of the outer membrane.

2. The gram-negative bacterial cell according to claim 1, wherein the heterologous peptide comprises 5 to 150 amino acid residues.

3. The gram-negative bacterial cell according to claim 1,
wherein the heterologous peptide is a growth factor, a pheromone, a hormone, a neuropeptide, a protease inhibitor, a self-assembling peptide, or a cell-signaling peptide.

4. The gram-negative bacterial cell according to claim 1, wherein the heterologous peptide is H. sapiens epidermal growth factor, an H. sapiens endorphin, S. cerevisiae α-factor, or H. medicinalis eglin C.

5. The gram-negative bacterial cell according to claim 1, wherein the secretion signal sequence is a microcin secretion signal sequence.

6. The gram-negative bacterial cell according to claim 1, wherein:
the secretion signal sequence comprises a sequence M-X₁-Xₘ-[B]-Xₙ-G-[J],
M is methionine,
G is glycine,
each residue “X” is independently any amino acid,
subscript 1 is an integer ranging from 0 to 9, subscript m is 2, and subscript n is 9,
residue “B” is isoleucine or leucine, and
residue “J” is alanine or glycine, wherein residue “J” is fused to the heterologous peptide.

7. The gram-negative bacterial cell according to claim 1, wherein the PCAT is E coli CvaB.

8. The gram-negative bacterial cell according to claim 1, wherein the first nucleic acid and the second nucleic acid are operably linked to an inducible promoter or a constitutive promoter,

9. The gram-negative bacterial cell according to claim 1, wherein the first nucleic acid is operably linked to a first promoter and the second nucleic acid is operably linked to a second promoter.

10. The gram-negative bacterial cell according to claim 1, further comprising a fourth nucleic encoding an outer membrane channel protein.

11. (canceled)

12. A method for preparing a peptide, the method comprising: culturing a gram-negative bacterial cell according to claim 1 such that the heterologous peptide is expressed and secreted from the cytosol to the external environment; and isolating the secreted heterologous peptide, thereby preparing the peptide.

13. A method for delivering a peptide from gram-negative bacterial cytosol to an external environment, the method comprising introducing a gram-negative cell according to claim 1 to the external environment, such that the gram-negative bacterial cell expresses and secretes the heterologous peptide from the cytosol to the external environment, thereby delivering the peptide.

14. The method of claim 12, wherein the external environment comprises a tissue or organ of a human subject or animal subject.

15. The method of claim 12, wherein the external environment is an agricultural environment.

16. A gram-negative epiphytic or soil bacterial cell comprising:
a first nucleic acid encoding a secretion signal sequence fused to a heterologous agricultural peptide, a second nucleic acid encoding a C39 peptidase-containing ATP-binding cassette transporter (PCAT), and a third nucleic acid encoding a membrane fusion protein; and
an inner membrane surrounding cytosol, an outer membrane surrounding the inner membrane, and periplasmic space between the inner membrane and the outer membrane;
wherein the gram-negative bacterial cell secretes the heterologous agricultural peptide from the bacterial cytosol to an external environment outside of the outer membrane.

17. The gram-negative epiphytic or soil bacterial cell according to claim 16, wherein the heterologous agricultural peptide comprises 5 to 150 amino acid residues.

18. The gram-negative epiphytic or soil bacterial cell according to claim 16, wherein the heterologous agricultural peptide is an antifungal peptide, an antibacterial peptide, a plant hormone, or a plant growth regulator.

19. (canceled)

20. The gram-negative epiphytic or soil bacterial cell according to claim 16, wherein the secretion signal sequence is a microcin secretion signal sequence.

21. The gram-negative epiphytic or soil bacterial cell according to claim 19, wherein:
the secretion signal sequence comprises a sequence M-X₁-Xₘ-[B]-Xₙ-G-[J],
M is methionine,
G is glycine,
each residue “X” is independently any amino acid,
subscript 1 is an integer ranging from 0 to 9, subscript m is 2, and subscript n is 9,
residue “B” is isoleucine or leucine, and
residue “J” is alanine or glycine, wherein residue “J” is fused to the heterologous agricultural peptide.

22. (canceled)

23. The gram-negative epiphytic or soil bacterial cell according to claim 16, wherein the epiphytic bacterial cell is a Gilliamella, Panteoa, Paraburkholdera, Serratia, Pseudomonas, Rhizobium or Bradyrhizobium cell.

24. (canceled)

25. (canceled)

26. (canceled)

27. (canceled)

28. The gram-negative epiphytic or soil bacterial cell according to claim 16, wherein the PCAT is selected from the group consisting of a Gilliamella apiciola PCAT, a Gilliamella apis PCAT, a Panteoa vagans PCAT, a Paraburkholdera xenovorans PCAT, or a Serratia plymuthica PCAT.

29. The gram-negative epiphytic or soil bacterial cell according to claim 16, wherein the signal sequence and/or the PCAT are from the same bacterial species as the epiphytic bacterial cell.

30. The gram-negative epiphytic or soil bacterial cell according claim 16, wherein the bacterial cell is a Gilliamella apiciola cell, wherein the signal sequence is MKELNLIEVEQVSGA (SEQ ID NO: 673), and wherein the PCAT comprises SEQ ID NO: 679; wherein the bacterial cell is a Gilliamella apis cell, wherein the signal sequence is a MKELNKVEVEQVSGA (SEQ ID NO: 674), and wherein the PCAT comprises SEQ ID NO: 680, wherein the bacterial cell is a Panteoa vagans cell, wherein the signal sequence is a MRELTSVEMQNVSGA (SEQ ID NO: 675) or MRELKTNEIDGVSGG (SEQ ID NO: 676), and wherein the PCAT comprises SEQ ID NO: 681, wherein the bacterial cell is a Paraburkholdera xenovorans cell, wherein the signal sequence is a MRELTSYELQAVSGG (SEQ ID NO: 677), and wherein the PCAT comprises SEQ ID NO: 682, or wherein the bacterial cell is a Serratia plymuthica cell, wherein the signal sequence is a MRELTSYELQAVSGG (SEQ ID NO: 678), and wherein the PCAT comprises SEQ ID NO: 94.

31. (canceled)

32. (canceled)

33. (canceled)

34. (canceled)

35. The gram-negative epiphytic or soil bacterial cell according to claim 16, wherein the first nucleic acid and the second nucleic acid are operably linked to an inducible promoter or a constitutive promoter,

36. The gram-negative epiphytic or soil bacterial cell according to claim 16, wherein the first nucleic acid is operably linked to a first promoter and the second nucleic acid is operably linked to a second promoter.

37. The gram-negative epiphytic or soil bacterial cell according to claim 16, further comprising a fourth nucleic encoding an outer membrane channel protein.

38. A method for preparing a peptide, the method comprising: culturing a gram-negative epiphytic or soil bacterial cell according to claim 16, such that the heterologous agricultural peptide is expressed and secreted from the cytosol to the external environment; and isolating the secreted heterologous agricultural peptide, thereby preparing the peptide.

39. A method for delivering a heterologous agricultural peptide from a gram-negative epiphytic or soil bacteria cytosol to a plant, the method comprising contacting the plant with the gram-negative epiphytic or soil bacterial cell according to claim 16, such that the gram-negative epiphytic bacterial cell expresses and secretes the heterologous peptide from the cytosol to the plant, thereby delivering the peptide.

40. A method for colonizing the gastrointestinal tract of a subject with bacteria expressing a therapeutic protein comprising administering to a subject in need thereof, an effective amount of the gram-negative bacteria of claim 1 to the subject.

41. The method of claim 40, wherein at least a portion of the population of bacterial cells present in the gastrointestinal tract of the subject is displaced as a result of colonization with the gram-negative bacteria.

42. A method for treating a disease or disorder associated with or affected by gut microbiota in a subject comprising administering to the subject with the disease or disorder an effective amount of the gram-negative bacteria of claim 1.

43. (canceled)

44. The method of claim 40, wherein the gram-negative bacteria are orally administered to the subject.

45. A method for decreasing gastrointestinal inflammation in a subject comprising administering to the subject with gastrointestinal inflammation an effective amount of the gram-negative bacteria of claim 1 to the subject
